SEQUENCE LISTING (120 sequences)

SEQ ID NO: 1 - protein, 247 aa, synthetic construct
  VARIANT 20: note = Cys or Ser
  VARIANT 52: note = Met or Tyr
  VARIANT 54: note = Ser or Thr
  VARIANT 56: note = Thr or Glu
  VARIANT 156: note = Asp or Glu
  VARIANT 158: note = Leu or Met
  VARIANT 228: note = Met or Leu
  VARIANT 234: note = Asn or Ser
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 2 - protein, 247 aa, synthetic construct
  VARIANT 52: note = Met or Tyr
  VARIANT 54: note = Ser or Thr
  VARIANT 56: note = Thr or Glu
  VARIANT 156: note = Asp or Glu
  VARIANT 158: note = Leu or Met
  VARIANT 228: note = Met or Leu
  VARIANT 234: note = Asn or Ser
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 3 - protein, 247 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 7 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 8 - protein, 247 aa, synthetic construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 247 aa, synthetic construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 246 aa, synthetic construct
  VARIANT 20: note = Cys or Ser
  VARIANT 52: note = Met or Tyr
  VARIANT 54: note = Ser or Thr
  VARIANT 56: note = Thr or Glu
  VARIANT 156: note = Asp or Glu
  VARIANT 158: note = Leu or Met
  VARIANT 228: note = Met or Leu
  VARIANT 234: note = Asn or Ser
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 11 - protein, 246 aa, synthetic construct
  VARIANT 52: note = Met or Tyr
  VARIANT 54: note = Ser or Thr
  VARIANT 56: note = Thr or Glu
  VARIANT 156: note = Asp or Glu
  VARIANT 158: note = Leu or Met
  VARIANT 228: note = Met or Leu
  VARIANT 234: note = Asn or Ser
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 12 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 246 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 246 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 246 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 16 - protein, 246 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 17 - protein, 246 aa, synthetic construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 246 aa, synthetic construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 246 aa, synthetic construct
  VARIANT 19: note = Cys or Ser
  VARIANT 51: note = Met or Tyr
  VARIANT 53: note = Ser or Thr
  VARIANT 55: note = Thr or Glu
  VARIANT 155: note = Asp or Glu
  VARIANT 157: note = Leu or Met
  VARIANT 227: note = Met or Leu
  VARIANT 233: note = Asn or Ser
VNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 20 - protein, 246 aa, synthetic construct
  VARIANT 51: note = Met or Tyr
  VARIANT 53: note = Ser or Thr
  VARIANT 55: note = Thr or Glu
  VARIANT 155: note = Asp or Glu
  VARIANT 157: note = Leu or Met
  VARIANT 227: note = Met or Leu
  VARIANT 233: note = Asn or Ser
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 21 - protein, 246 aa, synthetic construct
  VARIANT 155: note = Asp or Glu
  VARIANT 157: note = Leu or Met
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 246 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 246 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 246 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 25 - protein, 246 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 26 - protein, 246 aa, synthetic construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 246 aa, synthetic construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 245 aa, synthetic construct
  VARIANT 19: note = Cys or Ser
  VARIANT 51: note = Met or Tyr
  VARIANT 53: note = Ser or Thr
  VARIANT 55: note = Thr or Glu
  VARIANT 155: note = Asp or Glu
  VARIANT 157: note = Leu or Met
  VARIANT 227: note = Met or Leu
  VARIANT 233: note = Asn or Ser
VNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 29 - protein, 245 aa, synthetic construct
  VARIANT 51: note = Met or Tyr
  VARIANT 53: note = Ser or Thr
  VARIANT 55: note = Thr or Glu
  VARIANT 155: note = Asp or Glu
  VARIANT 157: note = Leu or Met
  VARIANT 227: note = Met or Leu
  VARIANT 233: note = Asn or Ser
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 30 - protein, 245 aa, synthetic construct
  VARIANT 155: note = Asp or Glu
  VARIANT 157: note = Leu or Met
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 245 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 245 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 245 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 34 - protein, 245 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 35 - protein, 245 aa, synthetic construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 245 aa, synthetic construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 247 aa, synthetic construct
  VARIANT 1: note = Asn or absent
  VARIANT 20: note = Cys or Ser
  VARIANT 52: note = Met or Tyr
  VARIANT 54: note = Ser or Thr
  VARIANT 56: note = Thr or Glu
  VARIANT 97: note = Asn or Ala
  VARIANT 109: note = Leu or Asp
  VARIANT 111: note = Gln or His
  VARIANT 156: note = Asp or Glu
  VARIANT 158: note = Leu or Met
  VARIANT 228: note = Met or Leu
  VARIANT 234: note = Asn or Ser
  VARIANT 247: note = Lys or absent
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 38 - protein, 247 aa, synthetic construct
  VARIANT 1: note = Asn or absent
  VARIANT 97: note = Asn or Ala
  VARIANT 109: note = Leu or Asp
  VARIANT 111: note = Gln or His
  VARIANT 156: note = Asp or Glu
  VARIANT 158: note = Leu or Met
  VARIANT 234: note = Asn or Ser
  VARIANT 247: note = Lys or absent
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHXHYTQKSLSLSPGK

SEQ ID NO: 39 - protein, 247 aa, synthetic construct
  VARIANT 1: note = Asn or absent
  VARIANT 97: note = Asn or Ala
  VARIANT 156: note = Asp or Glu
  VARIANT 158: note = Leu or Met
  VARIANT 247: note = Lys or absent
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 40 - protein, 247 aa, synthetic construct; identical to SEQ ID NO: 39
  VARIANT 97: note = Asn or Ala
  VARIANT 156: note = Asp or Glu
  VARIANT 158: note = Leu or Met

SEQ ID NO: 41 - protein, 247 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 42 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 43 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 44 - protein, 246 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 45 - protein, 246 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 46 - protein, 247 aa, synthetic construct; identical to SEQ ID NO: 42

SEQ ID NO: 47 - protein, 247 aa, synthetic construct; identical to SEQ ID NO: 43

SEQ ID NO: 48 - protein, 245 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 49 - protein, 245 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 50 - protein, 247 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 51 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 52 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 53 - protein, 246 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 54 - protein, 246 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 55 - protein, 247 aa, synthetic construct; identical to SEQ ID NO: 51

SEQ ID NO: 56 - protein, 247 aa, synthetic construct; identical to SEQ ID NO: 52

SEQ ID NO: 57 - protein, 245 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 58 - protein, 245 aa, synthetic construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 59 - protein, 247 aa, synthetic construct
  VARIANT 1: note = Asn or absent
  VARIANT 97: note = Asn or Ala
  VARIANT 109: note = Leu or Asp
  VARIANT 111: note = Gln or His
  VARIANT 156: note = Asp or Glu
  VARIANT 158: note = Leu or Met
  VARIANT 234: note = Asn or Ser
  VARIANT 247: note = Lys or absent
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHXHYTQKSLSLSPGK

SEQ ID NO: 60 - protein, 247 aa, synthetic construct
  VARIANT 1: note = Asn or absent
  VARIANT 97: note = Asn or Ala
  VARIANT 156: note = Asp or Glu
  VARIANT 158: note = Leu or Met
  VARIANT 247: note = Lys or absent
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 61 - protein, 247 aa, synthetic construct; identical to SEQ ID NO: 60
  VARIANT 97: note = Asn or Ala
  VARIANT 156: note = Asp or Glu
  VARIANT 158: note = Leu or Met

SEQ ID NO: 62 - protein, 247 aa, synthetic construct
  VARIANT 156: note = Asp or Glu
  VARIANT 158: note = Leu or Met
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 63 - protein, 247 aa, synthetic construct
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 64 - protein, 247 aa, synthetic construct
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 65 - protein, 246 aa, synthetic construct
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 66 - protein, 246 aa, synthetic construct
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 67 - protein, 247 aa, synthetic construct; identical to SEQ ID NO: 63

SEQ ID NO: 68 - protein, 247 aa, synthetic construct; identical to SEQ ID NO: 64

SEQ ID NO: 69 - protein, 245 aa, synthetic construct
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 70 - protein, 245 aa, synthetic construct
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 71 - protein, 247 aa, synthetic construct
  VARIANT 156: note = Asp or Glu
  VARIANT 158: note = Leu or Met
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 72 - protein, 247 aa, synthetic construct
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 73 - protein, 247 aa, synthetic construct
NVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 74 - protein, 246 aa, synthetic construct
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 75 - protein, 246 aa, synthetic construct
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 76 - protein, 247 aa, synthetic construct; identical to SEQ ID NO: 72

SEQ ID NO: 77 - protein, 247 aa, synthetic construct; identical to SEQ ID NO: 73

SEQ ID NO: 78 - protein, 245 aa, synthetic construct
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 79 - protein, 245 aa, synthetic construct
VNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 80 - protein, 246 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 246 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 246 aa, synthetic construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 246 aa, synthetic construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 247 aa, synthetic construct
  VARIANT 1: note = Asn or absent
  VARIANT 20: note = Cys or Ser
  VARIANT 46: note = Ser, Gly, Ala, Thr, Asn, Gln, Arg, His, Glu, or Asp
  VARIANT 52: note = Met or Tyr
  VARIANT 54: note = Ser or Thr
  VARIANT 56: note = Thr or Glu
  VARIANT 97: note = Asn or Ala
  VARIANT 109: note = Leu or Asp
  VARIANT 111: note = Gln or His
  VARIANT 156: note = Asp or Glu
  VARIANT 158: note = Leu or Met
  VARIANT 228: note = Met or Leu
  VARIANT 234: note = Asn or Ser
  VARIANT 247: note = Lys or absent
NVNHKPSNTKVDKKVEPKSXDKTHTCPPCPAPELLGGPSVFLFPPXPKDTLXIXRXPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 85 - protein, 247 aa, synthetic construct
  VARIANT 1: note = Asn or absent
  VARIANT 46: note = Ser, Gly, Ala, Thr, Asn, Gln, Arg, His, Glu, or Asp
  VARIANT 97: note = Asn or Ala
  VARIANT 156: note = Asp or Glu
  VARIANT 158: note = Leu or Met
  VARIANT 247: note = Lys or absent
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 246 aa, synthetic construct
  VARIANT 46: note = Ser, Gly, Ala, Thr, Asn, Gln, Arg, His, Glu, or Asp
  VARIANT 97: note = Asn or Ala
  VARIANT 156: note = Asp or Glu
  VARIANT 158: note = Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 246 aa, synthetic construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 246 aa, synthetic construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 246 aa, synthetic construct
  VARIANT 46: note = Ser, Gly, Ala, Thr, Asn, Gln, Arg, His, Glu, or Asp
  VARIANT 156: note = Asp or Glu
  VARIANT 158: note = Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 247 aa, synthetic construct
  VARIANT 1: note = Asn or absent
  VARIANT 46: note = Ser, Gly, Ala, Thr, Asn, Gln, Arg, His, Glu, or Asp
  VARIANT 97: note = Asn or Ala
  VARIANT 156: note = Asp or Glu
  VARIANT 158: note = Leu or Met
  VARIANT 247: note = Lys or absent
NVNHKPSNTKVDKKVEPKSSDKTHTCPPCPAPELLGGPSVFLFPPXPKDTLYITREPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 246 aa, synthetic construct
  VARIANT 46: note = Ser, Gly, Ala, Thr, Asn, Gln, Arg, His, Glu, or Asp
  VARIANT 97: note = Asn or Ala
  VARIANT 156: note = Asp or Glu
  VARIANT 158: note = Leu or Met
NVNHKPSNTKVDKKVEPKSSDKTHTCPPCPAPELLGGPSVFLFPPXPKDTLYITREPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 246 aa, synthetic construct
NVNHKPSNTKVDKKVEPKSSDKTHTCPPCPAPELLGGPSVFLFPPSPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 246 aa, synthetic construct
NVNHKPSNTKVDKKVEPKSSDKTHTCPPCPAPELLGGPSVFLFPPSPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 246 aa, synthetic construct
  VARIANT 46: note = Ser, Gly, Ala, Thr, Asn, Gln, Arg, His, Glu, or Asp
  VARIANT 156: note = Asp or Glu
  VARIANT 158: note = Leu or Met
NVNHKPSNTKVDKKVEPKSSDKTHTCPPCPAPELLGGPSVFLFPPXPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 247 aa, synthetic construct
  VARIANT 1: note = Asn or absent
  VARIANT 46: note = Ser, Gly, Ala, Thr, Asn, Gln, Arg, His, Glu, or Asp
  VARIANT 97: note = Asn or Ala
  VARIANT 156: note = Asp or Glu
  VARIANT 158: note = Leu or Met
  VARIANT 247: note = Lys or absent
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 100 - protein, 246 aa, synthetic construct
  VARIANT 46: note = Ser, Gly, Ala, Thr, Asn, Gln, Arg, His, Glu, or Asp
  VARIANT 97: note = Asn or Ala
  VARIANT 156: note = Asp or Glu
  VARIANT 158: note = Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 101 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 102 - protein, 246 aa, synthetic construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 103 - protein, 246 aa, synthetic construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 104 - protein, 246 aa, synthetic construct
  VARIANT 46: note = Ser, Gly, Ala, Thr, Asn, Gln, Arg, His, Glu, or Asp
  VARIANT 156: note = Asp or Glu
  VARIANT 158: note = Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 105 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 106 - protein, 247 aa, synthetic construct
  VARIANT 1: note = Asn or absent
  VARIANT 46: note = Ser, Gly, Ala, Thr, Asn, Gln, Arg, His, Glu, or Asp
  VARIANT 97: note = Asn or Ala
  VARIANT 156: note = Asp or Glu
  VARIANT 158: note = Leu or Met
  VARIANT 247: note = Lys or absent
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 107 - protein, 246 aa, synthetic construct
  VARIANT 46: note = Ser, Gly, Ala, Thr, Asn, Gln, Arg, His, Glu, or Asp
  VARIANT 97: note = Asn or Ala
  VARIANT 156: note = Asp or Glu
  VARIANT 158: note = Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 108 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 109 - protein, 246 aa, synthetic construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 110 - protein, 246 aa, synthetic construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 111 - protein, 246 aa, synthetic construct
  VARIANT 46: note = Ser, Gly, Ala, Thr, Asn, Gln, Arg, His, Glu, or Asp
  VARIANT 156: note = Asp or Glu
  VARIANT 158: note = Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 112 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 113 - protein, 468 aa, synthetic construct
MGWSCIILFLVATATGVHSQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 232 aa, synthetic construct
MGWSCIILFLVATATGVHSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 246 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 246 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 246 aa, synthetic construct
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 118 - protein, 246 aa, synthetic construct
  VARIANT 46: note = Ser, Gly, Ala, Thr, Asn, Gln, Arg, His, Glu, or Asp
  VARIANT 156: note = Asp or Glu
  VARIANT 158: note = Leu or Met
NVNHKPSNTKVDKKVEPKSSDKTHTCPPCPAPELLGGPSVFLFPPXPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 119 - protein, 246 aa, synthetic construct
  VARIANT 156: note = Asp or Glu
  VARIANT 158: note = Leu or Met
NVNHKPSNTKVDKKVEPKSSDKTHTCPPCPAPELLGGPSVFLFPPSPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 120 - protein, 247 aa, synthetic construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0000781 A1
(43) Pub. Date: Jan. 1, 2026

(54) FC CONJUGATES INCLUDING AN INHIBITOR OF CD73 AND USES THEREOF

(71) Applicant: Cidara Therapeutics, Inc., San Diego, CA (US)

(72) Inventors: Allen BORCHARDT, San Diego, CA (US); Thomas P. BRADY, San Diego, CA (US); Hongyuan CHEN, Aliso Viejo, CA (US); Jason COLE, San Diego, CA (US); Ramkrishna DE, Vista, CA (US); Joanne M. FORTIER, San Diego, CA (US); Travis James HAUSSENER, San Diego, CA (US); Thanh LAM, San Diego, CA (US); Dhanya Raveendra PANICKAR, San Diego, CA (US); Leslie W. TARI, San Diego, CA (US)

(21) Appl. No.: 18/880,895

(22) PCT Filed: Jul. 5, 2023
(86) PCT No.: PCT/US2023/026932

§ 371 Date: Jan. 3, 2025

Related U.S. Application Data
(60) Provisional application No. 63/358,385, filed on Jul. 5, 2022.
(60) Provisional application No. 63/437,093, filed on Jan. 4, 2023.
(60) Provisional application No. 63/449,813, filed on Mar. 3, 2023.
(60) Provisional application No. 63/522,357, filed on Jun. 21, 2023.

(51) Int. Cl. A61K 47/68; A61K 9/00; A61K 39/00; A61K 45/06; A61P 35/00; C07K 16/28

(52) U.S. Cl. CPC A61K 47/6835; A61K 9/0019; A61K 45/06; A61K 47/6889; A61P 35/00; C07K 16/2818; A61K 2039/505

(57) ABSTRACT

The disclosure provides conjugates including an Fc domain monomer or Fc domain covalently linked to a moiety that binds to or inhibits CD73. The disclosure also provides pharmaceutical compositions including such conjugates and uses of such conjugates in the treatment of disorders associated with dysregulation or overexpression of CD73 (e.g., cancer, fibrosis, or a viral infection).

### BACKGROUND

Cluster of differentiation 73 (CD73) is a glycosyl phosphatidyl inositol-linked membrane protein found in most tissues that catalyzes the conversion of extracellular adenosine monophosphate (AMP) to adenosine. It functions as a homodimer and can be shed and is active as a soluble protein in circulation. In addition to its enzymatic function, CD73 is also a cellular adhesion molecule and plays a role in regulation of leukocyte trafficking.

In cancer, CD73 is expressed by many subsets of cells populating the tumor lesion, including tumor cells, stromal cells, and endothelial cells, as well as infiltrating immune cells. CD73 levels are known to be upregulated due to tissue injury or hypoxic conditions, and a number of solid tumors have elevated CD73 levels. Upregulation of CD73 within the tumor contributes to the adenosine-rich tumor microenvironment, which has numerous pro-tumor and immuno-suppressive effects. High CD73 tumor expression has been associated with shorter overall survival and poor prognosis in certain cancers. CD73 in cancer patients has also been associated with resistance to antitumor therapies.

Additionally, dysregulation of CD73 observed in various immune cell populations in viral infections suggests a functional role for purine nucleotide and nucleoside signaling in the context of immune responses against viral infections, including in SARS-CoV-2 viral infections.

CD73 dysregulation has also been shown to play a key role in pathogenesis of lung fibrosis induced by radiation therapy or other insults to lung tissue. In a mechanism unrelated to its catalytic activity, polyvalent ligation of CD73 enzymes has been shown to stimulate B cell activation, clonal expansion, and development of memory B-cells, suggesting that multivalent CD73 binding molecules could be used as adjuvants to enhance the efficacy of vaccines.

There is a need for novel treatments for disorders associated with CD73. The development of small molecule inhibitors of CD73 has been hindered by poor metabolic stability. In view of the role played by CD73 in cancer, as well as a diverse array of other diseases, disorders and conditions, and the current lack of CD73 inhibitors available to medical practitioners, new CD73 inhibitors, and compositions and methods associated therewith, are needed.

### SUMMARY

This disclosure relates to conjugates including an Fc domain monomer or Fc domain covalently linked to a moiety that binds to or inhibits CD73. In particular, such conjugates contain monomers or dimers of a moiety that binds to or inhibits CD73 conjugated to an Fc monomer or Fc domain. The Fc monomer or Fc domain in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC). This disclosure also provides pharmaceutical compositions including such conjugates and uses of such conjugates in the treatment of disorders associated with dysregulation or overexpression of CD73 (e.g., cancer, fibrosis, or a viral infection).

In an aspect, the disclosure features a conjugate, or a pharmaceutically acceptable salt thereof, described by formula (D-I) or (M-I):

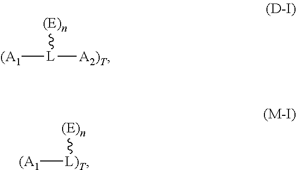

wherein each of A¹ and A², independently, has the structure of formula (A):

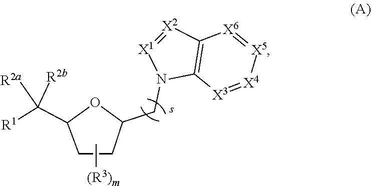

m is 0, 1, 2, 3, 4, 5, or 6;
s is 0 or 1;
each of X¹, X², X³, X⁴, X⁵, and X⁶ is, independently, N, CR⁴, or C—Y—R⁵, wherein at least one of X¹, X², X³, X⁴, X⁵, and X⁶ is C—Y—R⁵ and R⁵ is a bond to L;
R¹ is

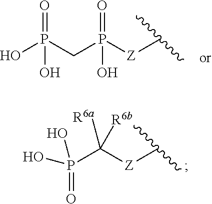

Z is O, S, or sulfonyl;
each of R^{2a} and R^{2b} is, independently, H, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl;
each R³ is, independently, OH, SH, halogen, optionally substituted amino, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl;
R⁴ is H, halogen, OH, SH, optionally substituted amino, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl;
each of R^{6a} and R^{6b} is, independently, H, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl;
Y is a first linker;
L is a second linker;
n is 1 or 2;
each E includes an Fc domain monomer;
T is an integer from 1 to 20; and
the squiggly line indicates that L is covalently attached to E.

In some embodiments, the conjugate is described by formula (D-I):

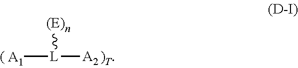

In some embodiments, the conjugate is described by formula (M-I):

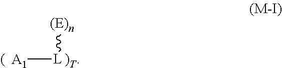

In some embodiments, A¹ and A² have the structure of formula (A-I):

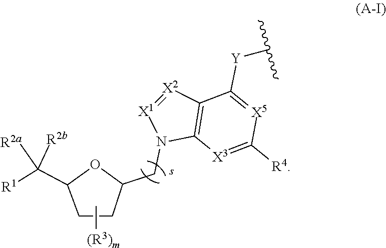

In some embodiments, A¹ and A² each have the structure of formula (A-Ia):

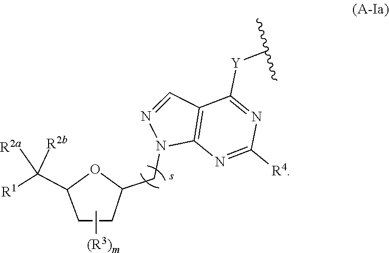

In some embodiments, A¹ and A² each have the structure of formula (A-Ib):

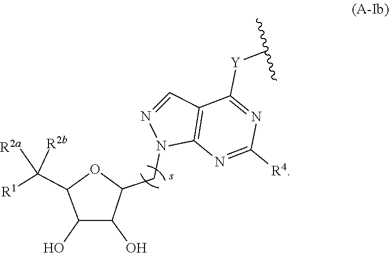

In some embodiments, A¹ and A² each have the structure of formula (A-Ib-1):

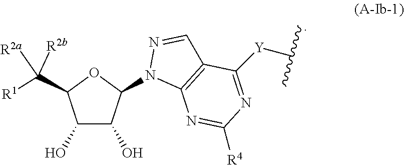

In some embodiments, A¹ and A² each have the structure of formula (A-Ib-2):

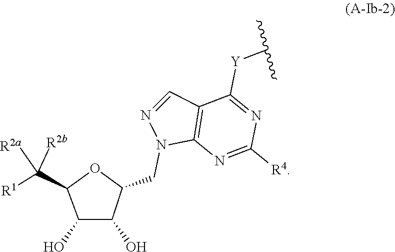

In some embodiments, A¹ and A² each have the structure of formula (A-II):

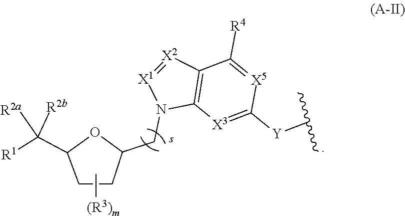

In some embodiments, A¹ and A² each have the structure of formula (A-IIa):

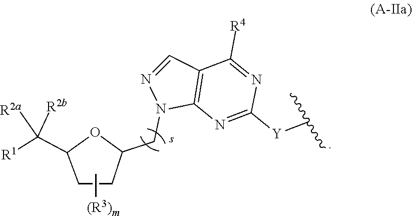

In some embodiments, A¹ and A² each have the structure of formula (A-IIb):

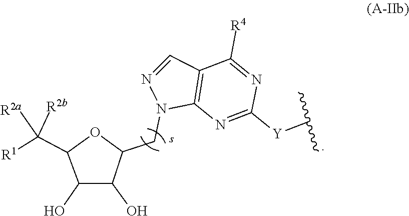

In some embodiments, A¹ and A² each have the structure of formula (A-IIb-1):

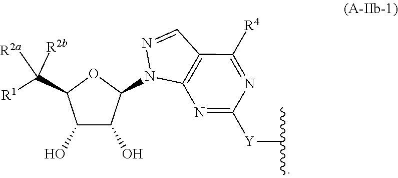

In some embodiments, A¹ and A² each have the structure of formula (A-IIb-2):

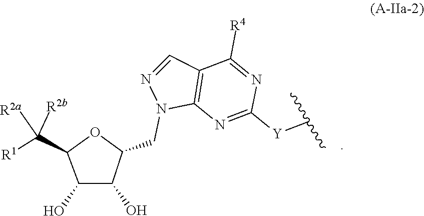

In some embodiments, s is 0. In some embodiments, s is 1.

In some embodiments, each of R^{2a} and R^{2b} is, independently, H, optionally substituted C₁-C₂₀ alkyl, or optionally substituted C₁-C₂₀ heteroalkyl. In some embodiments, each of R^{2a} and R^{2b} is H.

In some embodiments, R⁴ is H, halogen, OH, SH, optionally substituted amino, optionally substituted C₁-C₂₀ alkyl, or optionally substituted C₁-C₂₀ heteroalkyl. In some embodiments, R⁴ is halogen. In some embodiments, R⁴ is Cl.

In some embodiments, R¹ is

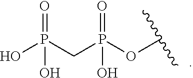 [SMILES: *OP(=O)(O)CP(=O)(O)O]

In some embodiments, R¹ is

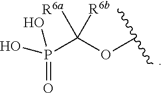

In some embodiments, each of R^{6a} and R^{6b} is, independently, H, optionally substituted C₁-C₂₀ alkyl, or optionally substituted C₁-C₂₀ heteroalkyl. In some embodiments, each of R^{6a} and R^{6b} is, independently, H, —CH₃, —CH₂CH₃, —CH₂OH, —CH₂OCH₃—CH₂CH₂OH, or —CH₂CH₂OCH₃. In some embodiments, each of R^{6a} and R^{6b} is H.

In some embodiments Y is:

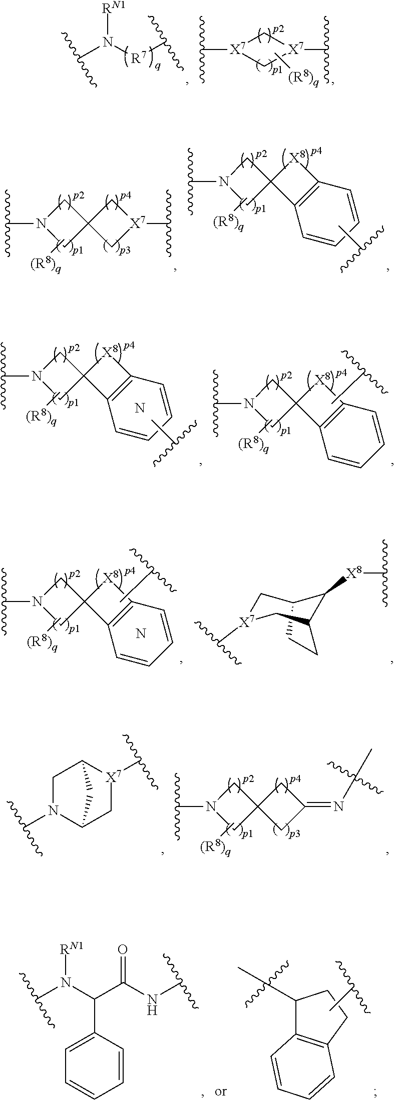

each of p1, p2, p3, and p4 is, independently, 0, 1, 2, 3, or 4;
q is 0, 1, 2, 3, or 4;
each X⁷ is, independently, N or CH;
each X⁸ is, independently, O, NH, CH₂, or C(═O);
R^{N1} is H, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, optionally substituted C₂-C₁₉ heteroaryl, optionally substituted C₁-C₂₀ alkaryl, or optionally substituted C₁-C₂₀ alkylcycloalkyl;
each R⁷ is, independently,

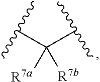

optionally substituted C₃-C₂₀ cycloalkyl, optionally substituted C₃-C₂₀ cycloalkenyl, optionally substituted C₂-C₂₀ heterocycloalkyl, or optionally substituted C₂-C₂₀ heterocycloalkenyl;
each of R^{7a} and R^{7b} is, independently, H, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl; and
each R⁸ is, independently, halogen, OH, SH, optionally substituted amino, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl.

In some embodiments, Y is

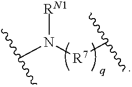

In some embodiments, q is 1 and Y is

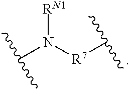

In some embodiments, R⁷ is

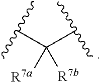

### and Y is

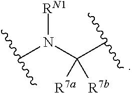

In some embodiments, Y is

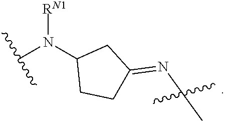

In some embodiments, q is 0 and Y is

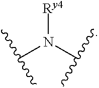

In some embodiments, R^{N1} is H,

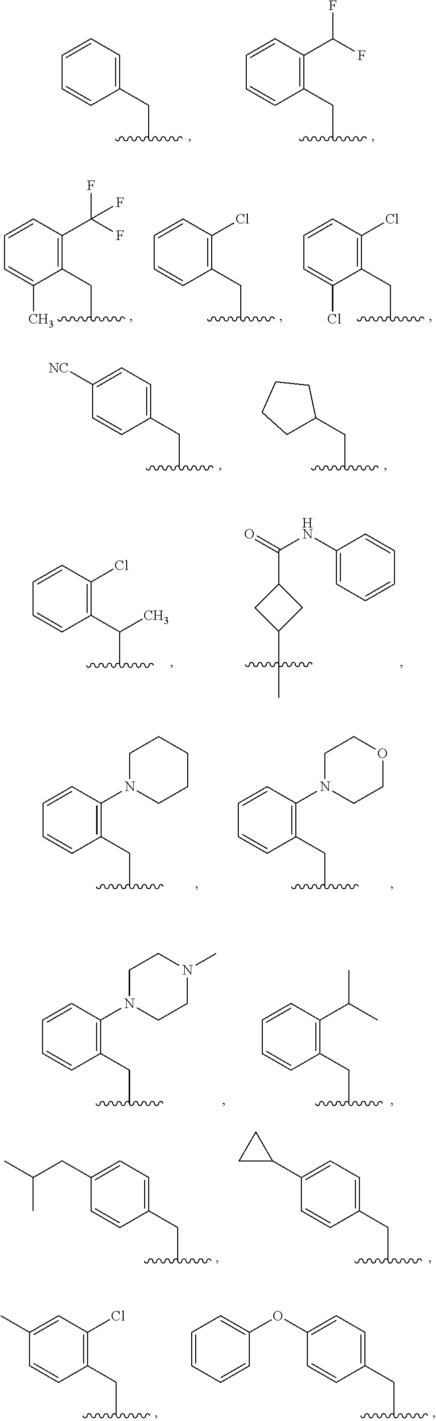

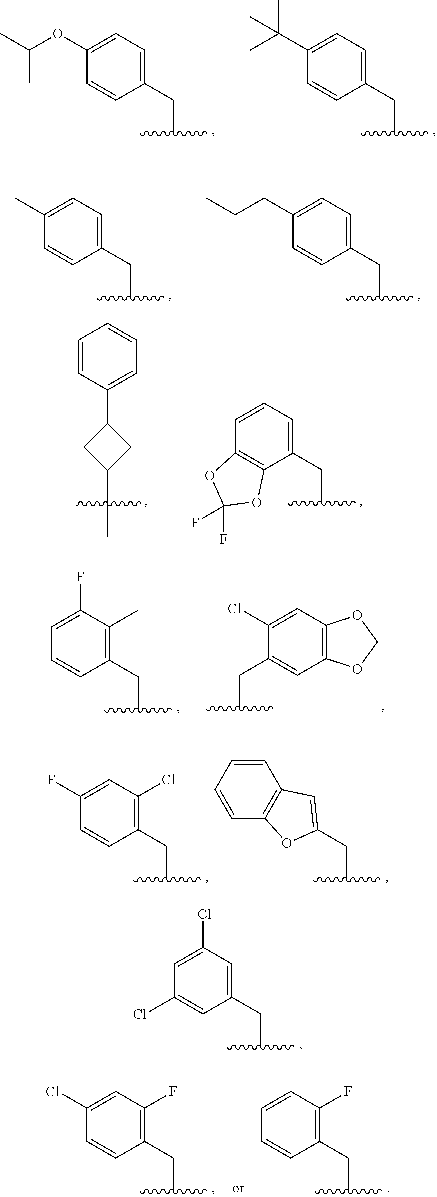

In some embodiments, Y is

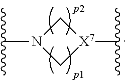

In some embodiments, Y is

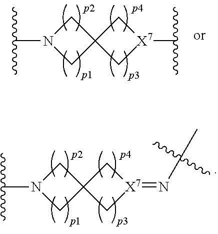

In some embodiments, Y is

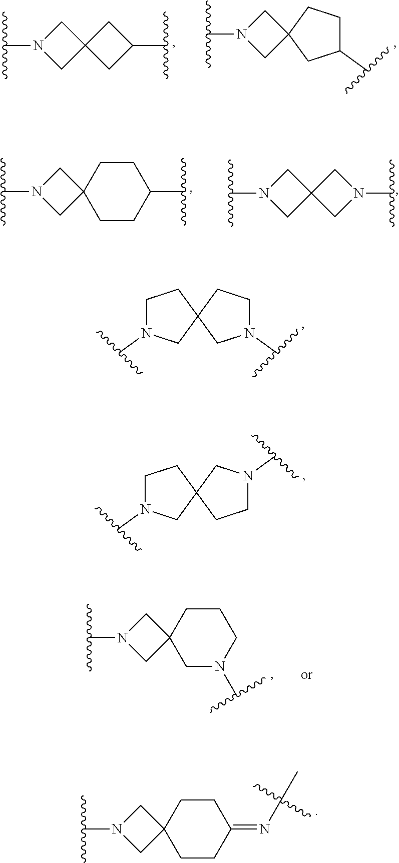

In some embodiments, Y is

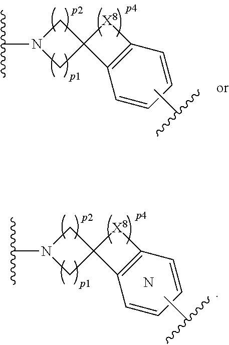

In some embodiments, Y is

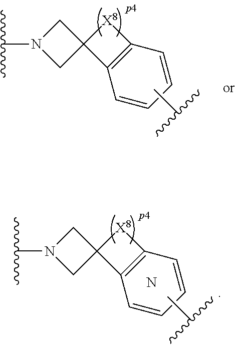

In some embodiments, Y is

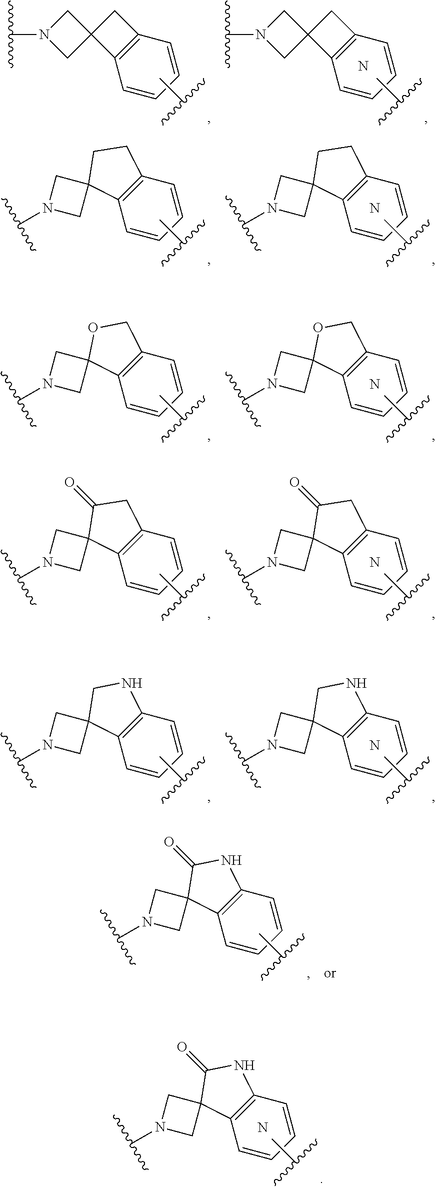

In some embodiments, Y is

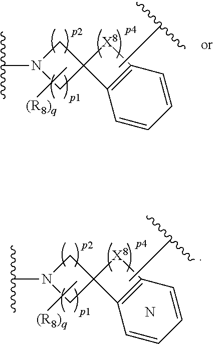

In some embodiments, Y is

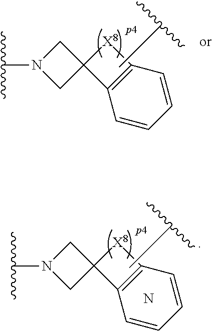

In some embodiments, Y is

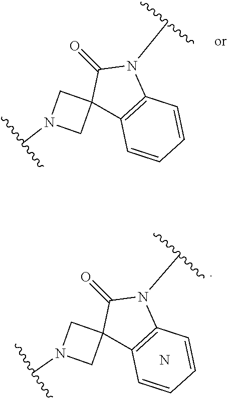

In some embodiments, Y is

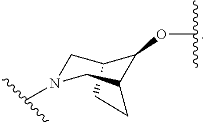

In some embodiments, L includes one or more optionally substituted C₁-C₄₀ alkylene, optionally substituted C₁-C₄₀ heteroalkylene, optionally substituted C₁-C₄₀ alkoxylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene, optionally substituted C₂-C₁₅ heteroarylene, O, S, NRⁱ, P, carbonyl, thiocarbonyl, sulfonyl, phosphate, phosphoryl, or imino,

wherein Rⁱ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C8-C20 heterocycloalkynyl, optionally substituted C₅-C₁₅ aryl, or optionally substituted C₂-C₁₅ heteroaryl.

In some embodiments, L is oxo substituted. In some embodiments, L includes between 1 and 250 atoms. In some embodiments, L is capable of forming an amide, a carbamate, a sulfonyl, or a urea linkage.

In some embodiments, L is described by the formula:

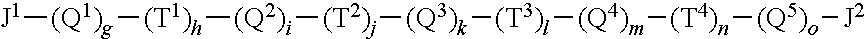

wherein J¹ is a bond attached to A₁;
J² is a bond attached to E or is a functional group capable of reacting with a functional group conjugated to E;
each of Q¹, Q², Q³, Q⁴ and Q⁵ is, independently, optionally substituted C1-C40 alkylene, optionally substituted C1-C40 heteroalkylene, optionally substituted C1-C40 alkoxylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene;
each of T¹, T², T³, T⁴ is, independently, O, S, NRⁱ, P, carbonyl, thiocarbonyl, sulfonyl, phosphate, phosphoryl, imino, or oximo;
Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C₅-C₁₅ aryl, or optionally substituted C₂-C₁₅ heteroaryl; and each of g, h, i, j, k, l, m, n, and o is, independently, 0, 1, or 2.

In some embodiments, Q¹ is O

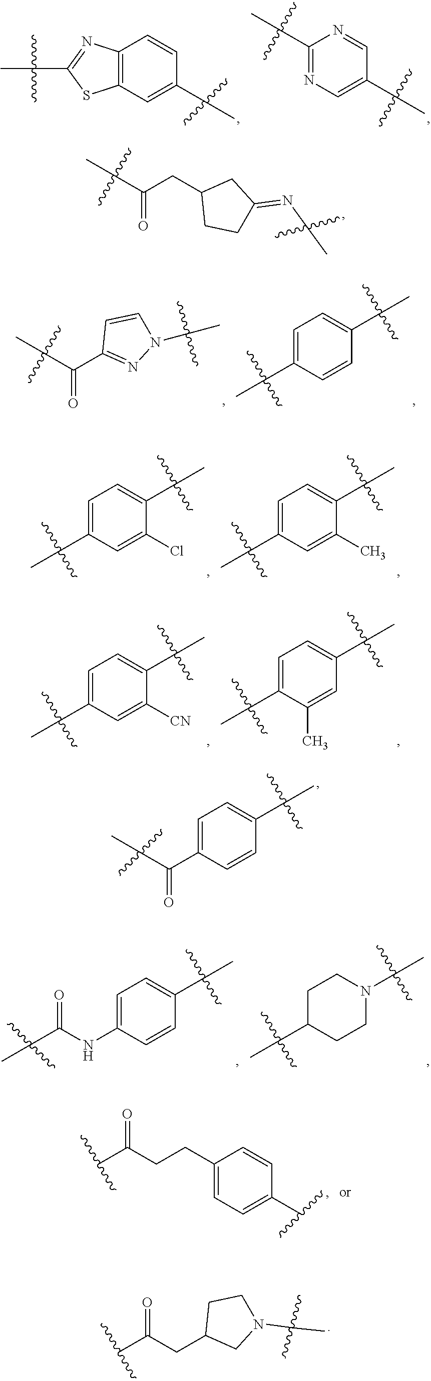

In some embodiments, Q² is optionally substituted C₁-C₄₀ alkylene, optionally substituted C₁-C₄₀ heteroalkylene, optionally substituted C₁-C₄₀ alkoxylene, or optionally substituted C₂-C₁₅ heteroarylene.

In some embodiments, Q³ is optionally substituted C₂-C₁₅ heteroarylene.

In some embodiments, Q⁴ is optionally substituted C₁-C₄₀ alkylene, optionally substituted C₁-C₄₀ heteroalkylene, or optionally substituted C₁-C₄₀ alkoxylene.

In some embodiments, J² is

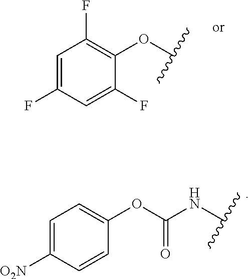 [SMILES: *Oc1c(F)cc(F)cc1F ; *NC(=O)Oc1ccc([N+](=O)[O-])cc1]

The disclosure features an intermediate (Int) of Table 1. These intermediates include one or more inhibitors of CD73 and a linker and may be used in the synthesis of a conjugate described herein. Intermediates of Table 1 may be conjugated to, for example, an Fc domain or Fc domain monomer (e.g., by way of a linker) by any suitable methods known to those of skill in the art, including any of the methods described or exemplified herein. In some embodiments, the conjugate includes E, wherein E is an Fc domain monomer or an Fc domain. In preferred embodiments, one or more nitrogen atoms of one or more surface exposed lysine residues of E or one or more sulfur atoms of one or more surface exposed cysteines in E is covalently conjugated to a linker (e.g., a PEG₂-PEG₂₀ linker). The linker conjugated to E may be functionalized such that it may react to form a covalent bond with any of the Ints described herein (e.g., an Int of Table 1). In preferred embodiments, E is conjugated to a linker functionalized with an azido group and the Int (e.g., an Int of Table 1) is functionalized with an alkyne group. Conjugation (e.g., by click chemistry) of the linker-azido of E and linker-alkyne of the Int forms a conjugate of the disclosure. In yet other embodiments, E is conjugated to a linker functionalized with an alkyne group and the Int (e.g., an Int of Table 1) is functionalized with an azido group. Conjugation (e.g., by click chemistry) of the linker-alkyne of E and the linker-azido of the Int forms a conjugate of the disclosure. In yet other embodiments, the Int (e.g., an Int of Table 2) is functionalized with a phenyl ester group (e.g., a trifluorophenyl ester group or a tetrafluorophenyl ester group). Conjugation (e.g., by acylation) of E and the linker-phenyl ester (e.g., trifluorophenyl ester or tetrafluorophenyl ester) of the Int forms a conjugate of the invention. Conjugation (e.g., by acylation) of E and the linker-phenyl ester (e.g., trifluorophenyl ester or tetrafluorophenyl ester) of the Int is conducted by methods described herein or known in the art.

The disclosure further features a composition (e.g., a pre-conjugation intermediate) having the structure of an Int of Table 1.

TABLE 1
Intermediates

| Inter- | |
|---|---|
| mediate | Structure |
| Int-1 | 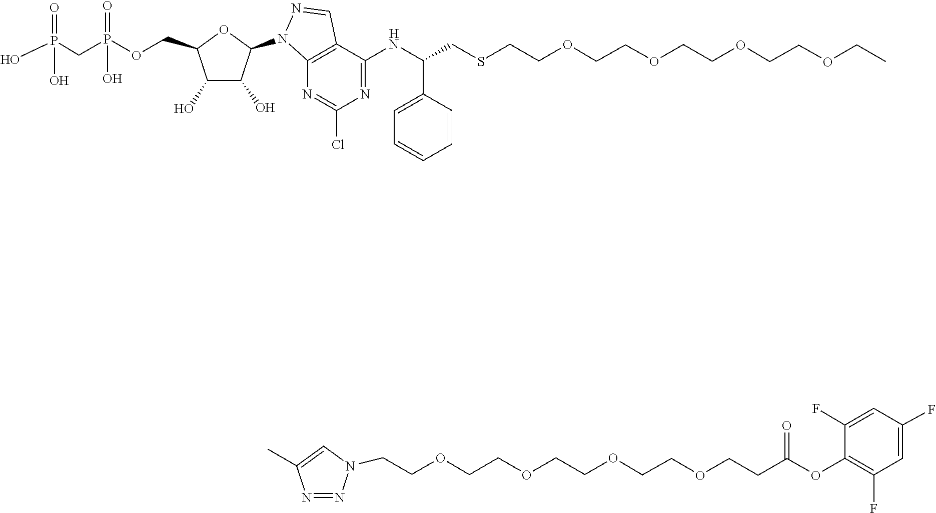 |
| Int-2 | 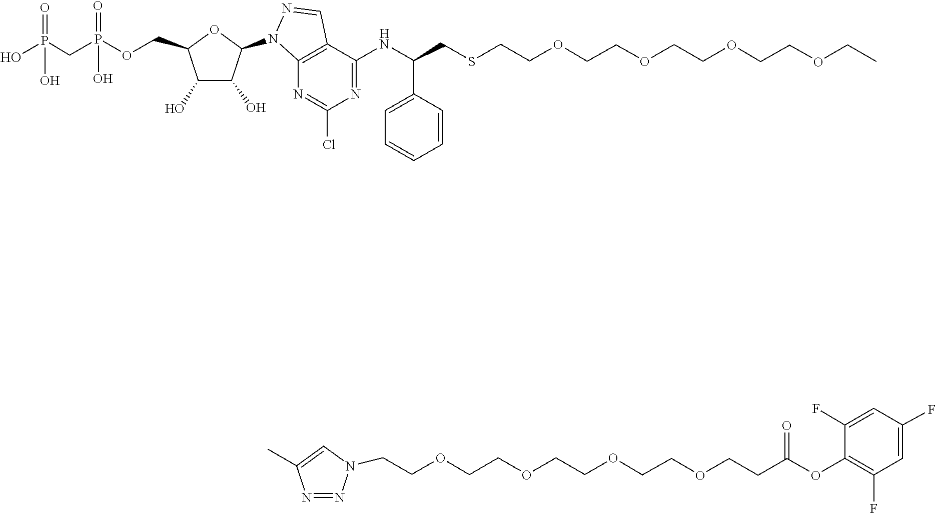 |
| Int-3 | 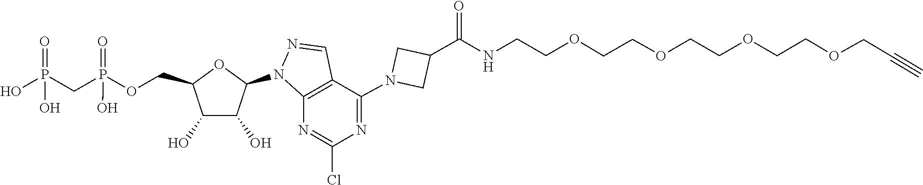 [SMILES: C#CCOCCOCCOCCOCCNC(=O)C1CN(c2nc(Cl)nc3c2cnn3[C@@H]2O[C@H](COP(=O)(O)CP(=O)(O)O)[C@@H](O)[C@H]2O)C1] |
| Int-4 | 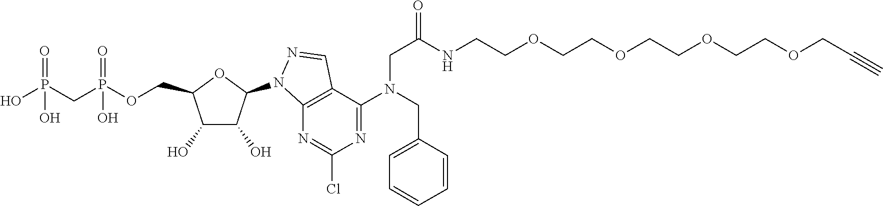 [SMILES: C#CCOCCOCCOCCOCCNC(=O)CN(Cc1ccccc1)c1nc(Cl)nc2c1cnn2[C@@H]1O[C@H](COP(=O)(O)CP(=O)(O)O)[C@@H](O)[C@H]1O] |
| Int-5 | 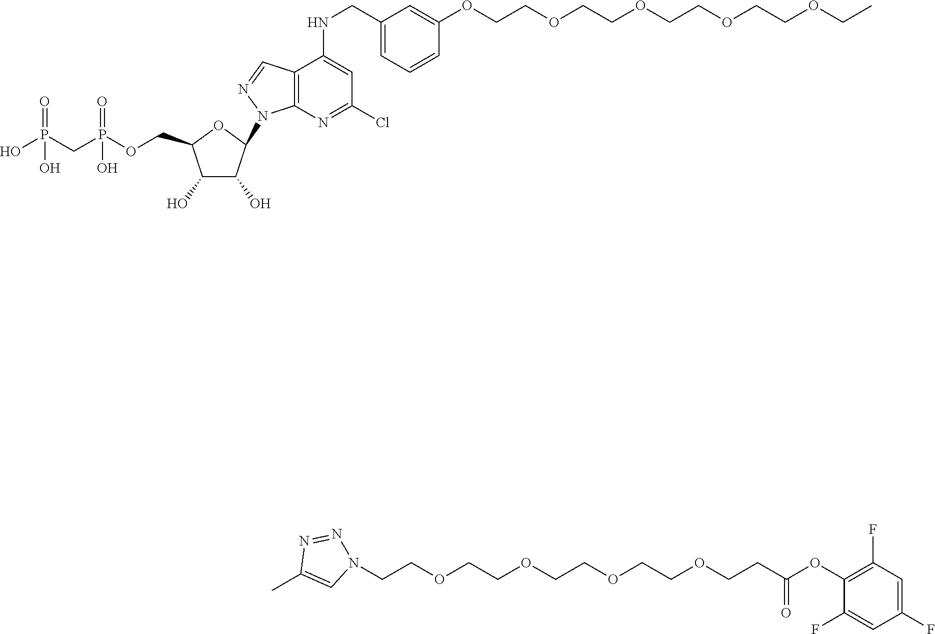 |
| Int-6 | 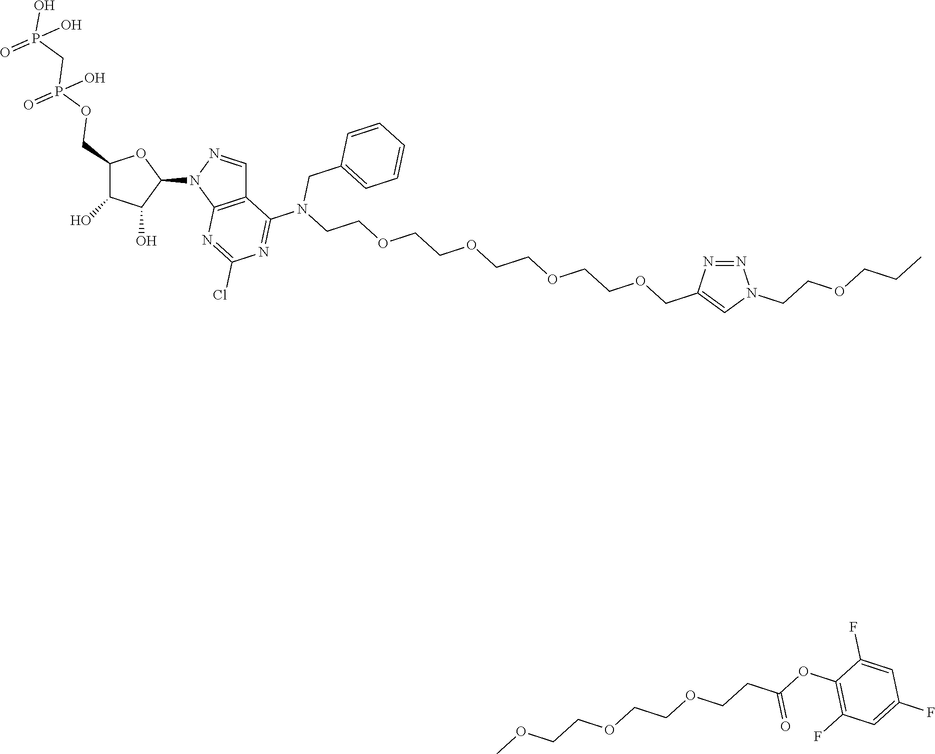 |
| Int-7 | 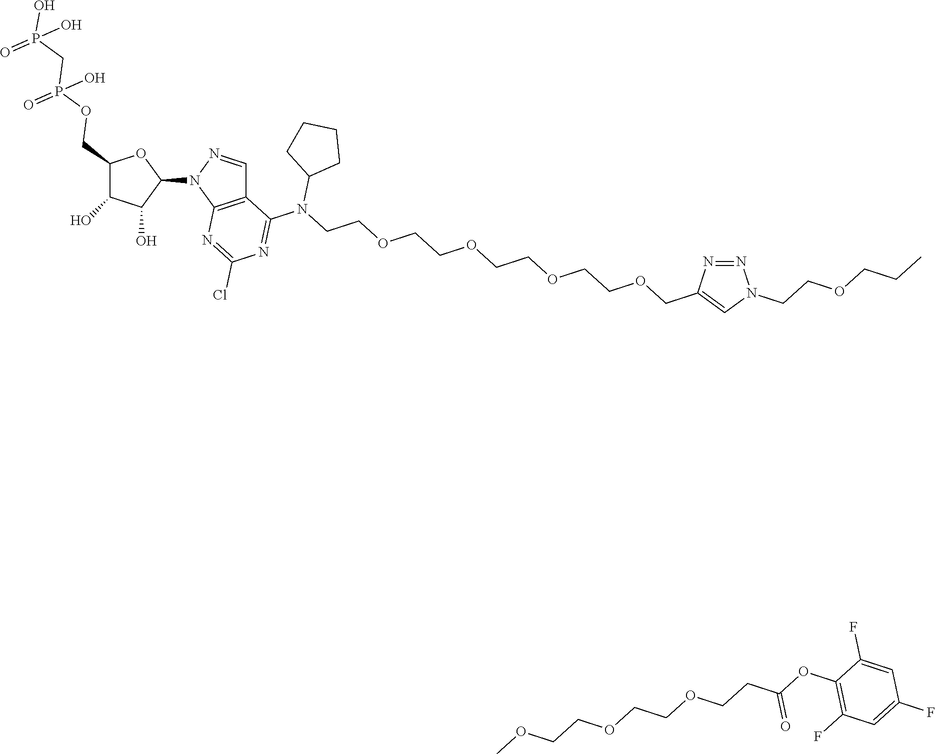 |
| Int-8 | 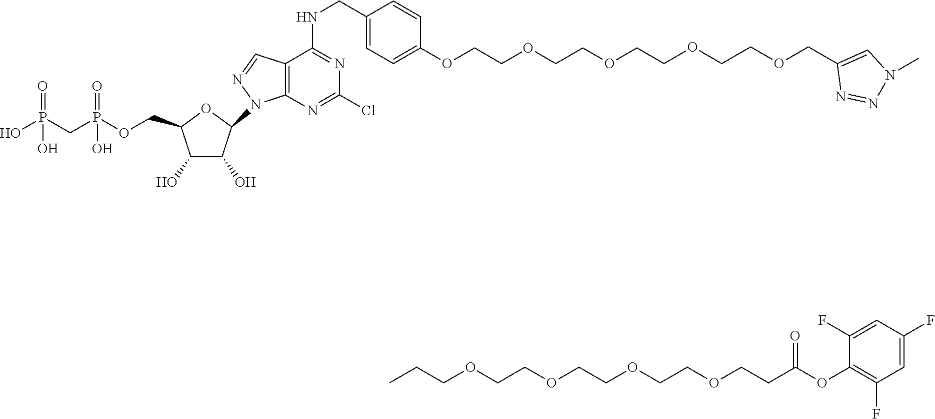 |
| Int-9 | 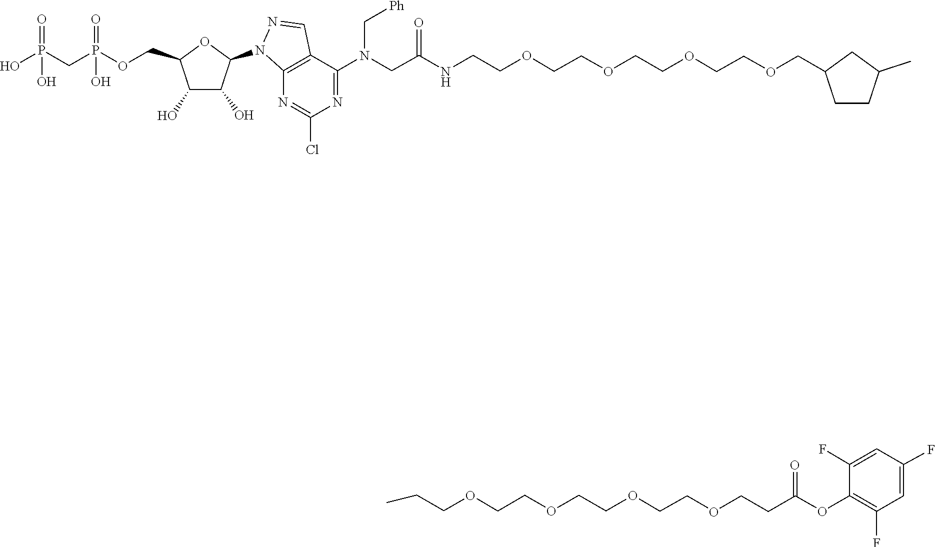 |
| Int-10 | 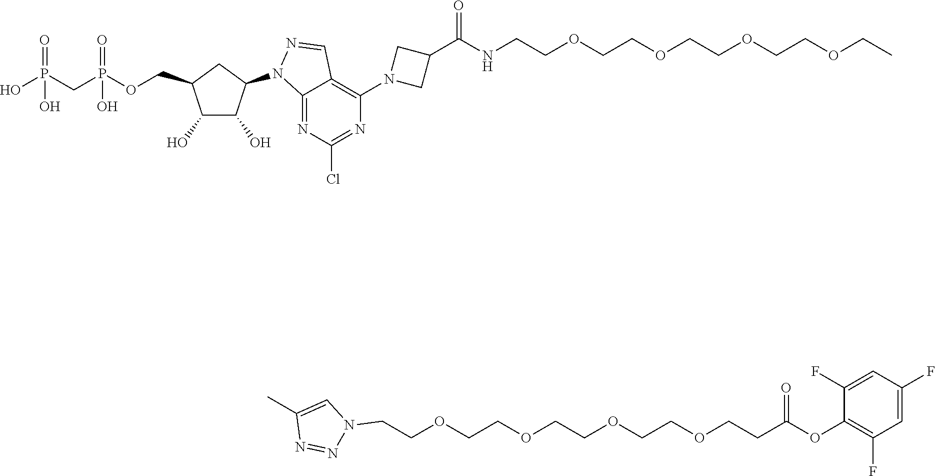 |
| Int-11 | 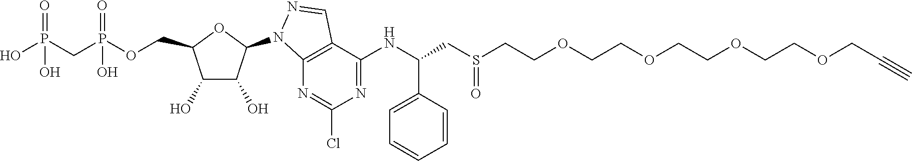 [SMILES: C#CCOCCOCCOCCOCCS(=O)C[C@@H](Nc1nc(Cl)nc2c1cnn2[C@@H]1O[C@H](COP(=O)(O)CP(=O)(O)O)[C@@H](O)[C@H]1O)c1ccccc1] |
| Int-12 | 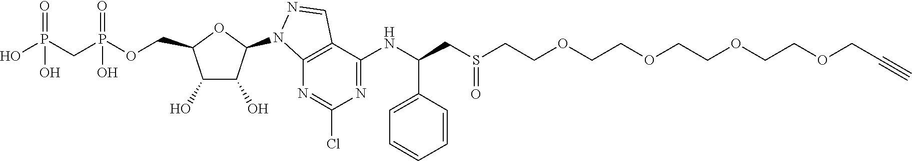 [SMILES: C#CCOCCOCCOCCOCCS(=O)C[C@H](Nc1nc(Cl)nc2c1cnn2[C@@H]1O[C@H](COP(=O)(O)CP(=O)(O)O)[C@@H](O)[C@H]1O)c1ccccc1] |
| Int-13 | 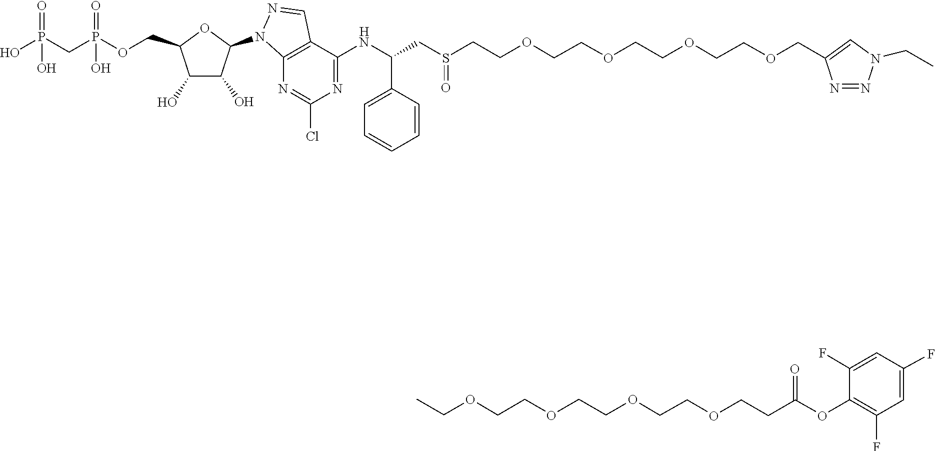 |
| Int-14 | 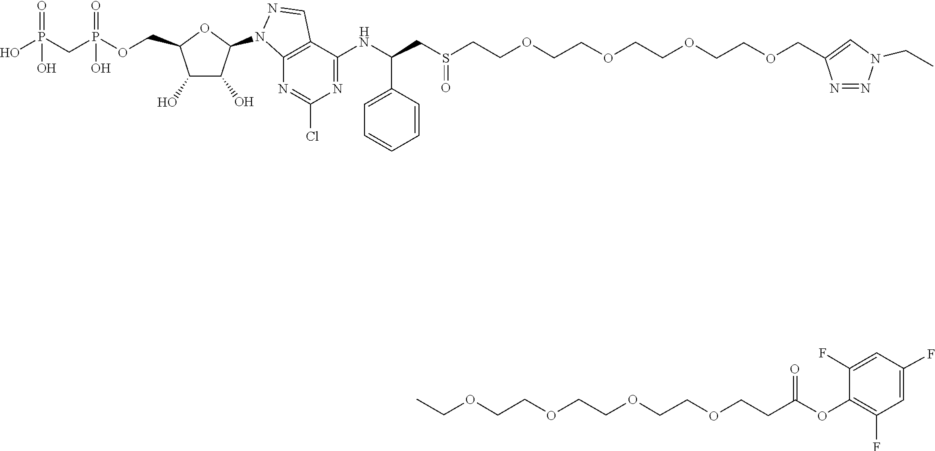 |
| Int-15 | 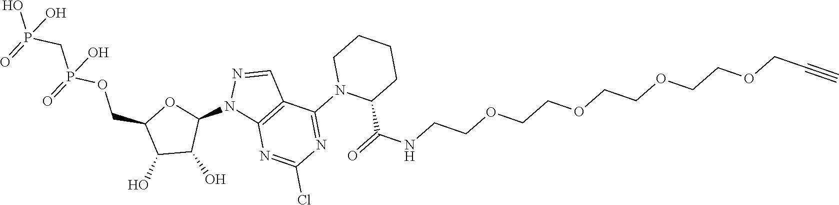 [SMILES: C#CCOCCOCCOCCOCCNC(=O)[C@H]1CCCCN1c1nc(Cl)nc2c1cnn2[C@@H]1O[C@H](COP(=O)(O)CP(=O)(O)O)[C@@H](O)[C@H]1O] |
| Int-16 | 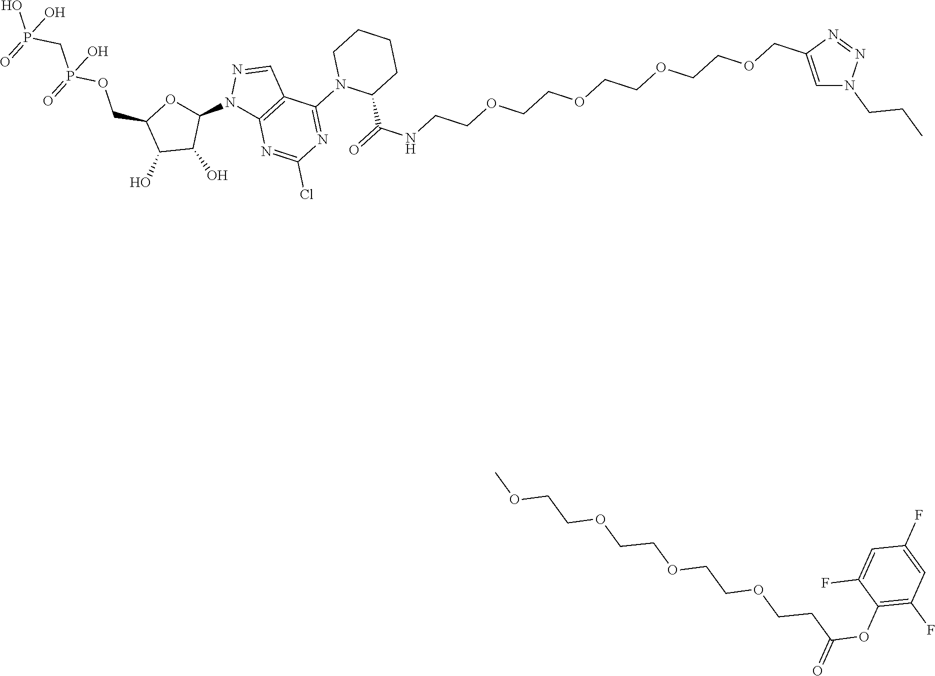 |
| Int-17 | 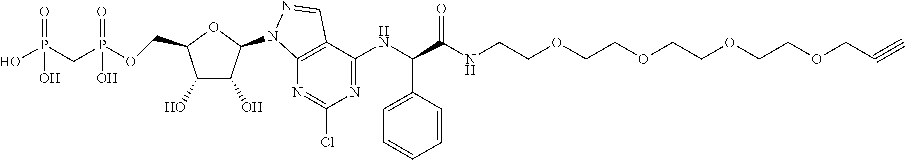 [SMILES: C#CCOCCOCCOCCOCCNC(=O)[C@H](Nc1nc(Cl)nc2c1cnn2[C@@H]1O[C@H](COP(=O)(O)CP(=O)(O)O)[C@@H](O)[C@H]1O)c1ccccc1] |
| Int-18 | 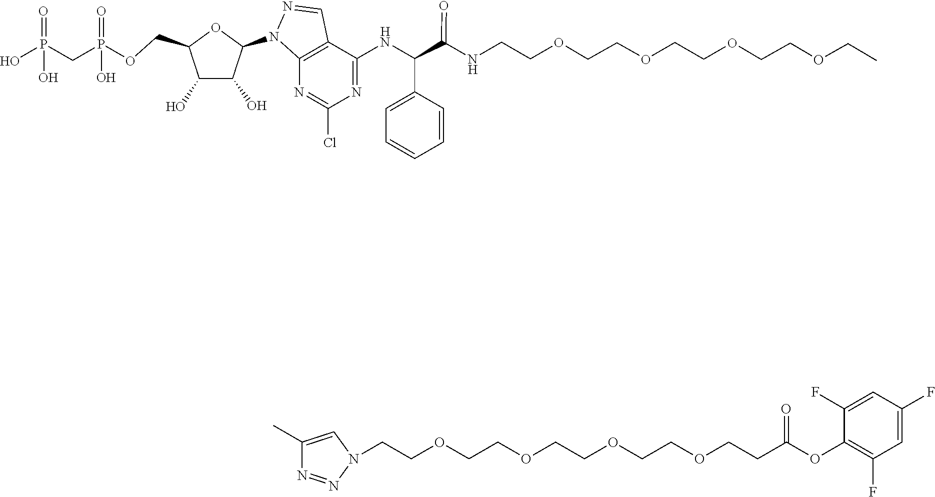 |
| Int-19 | 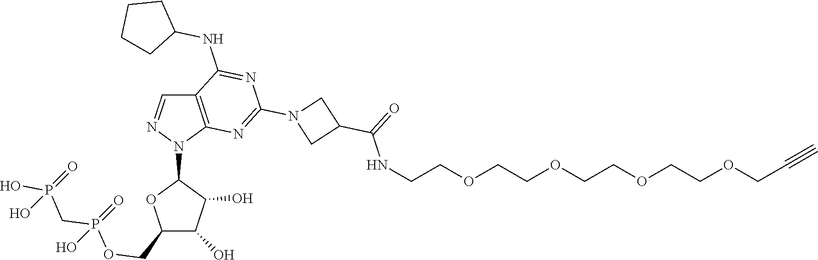 |
| Int-20 | 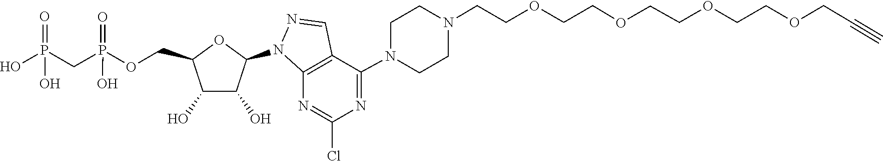 [SMILES: C#CCOCCOCCOCCOCCN1CCN(c2nc(Cl)nc3c2cnn3[C@@H]2O[C@H](COP(=O)(O)CP(=O)(O)O)[C@@H](O)[C@H]2O)CC1] |
| Int-21 | 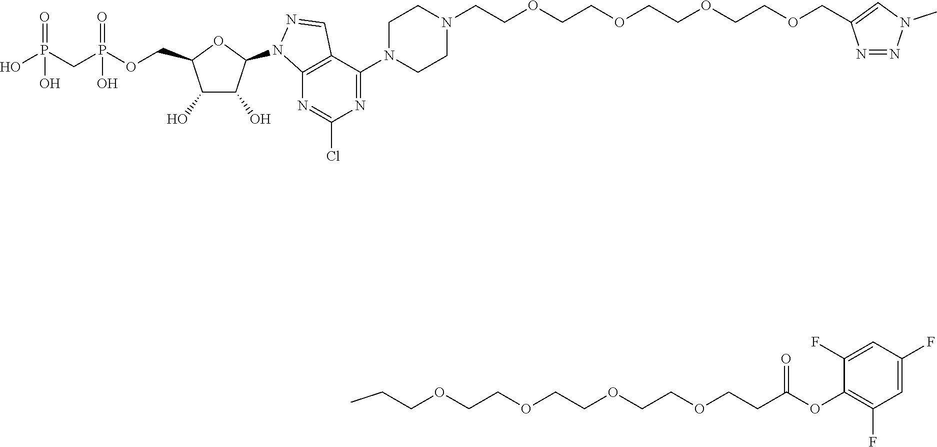 |
| Int-22 | 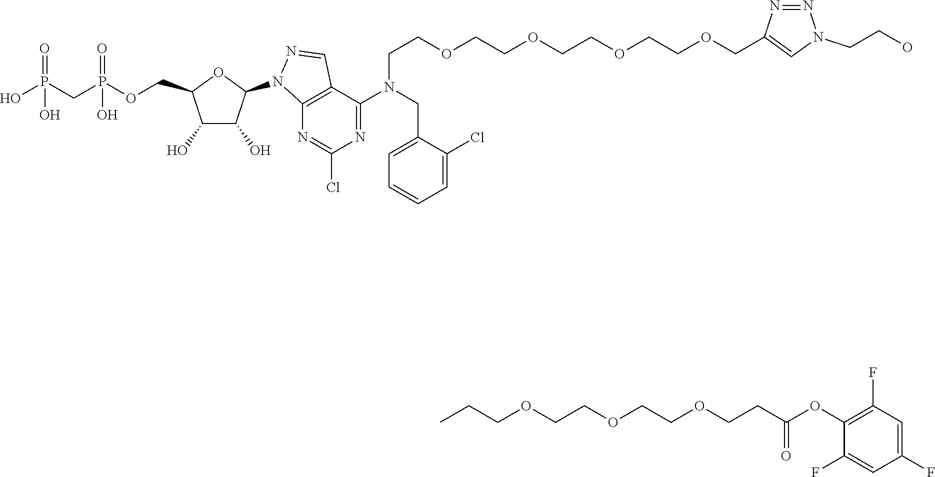 [SMILES: O=P(O)(O)CP(=O)(O)OC[C@H]1O[C@@H](n2ncc3c(N(CCOCCOCCOCCOCc4cn(CCO)nn4)Cc4ccccc4Cl)nc(Cl)nc32)[C@H](O)[C@@H]1O ; CCCOCCOCCOCCC(=O)Oc1c(F)cc(F)cc1F] |
| Int-23 | 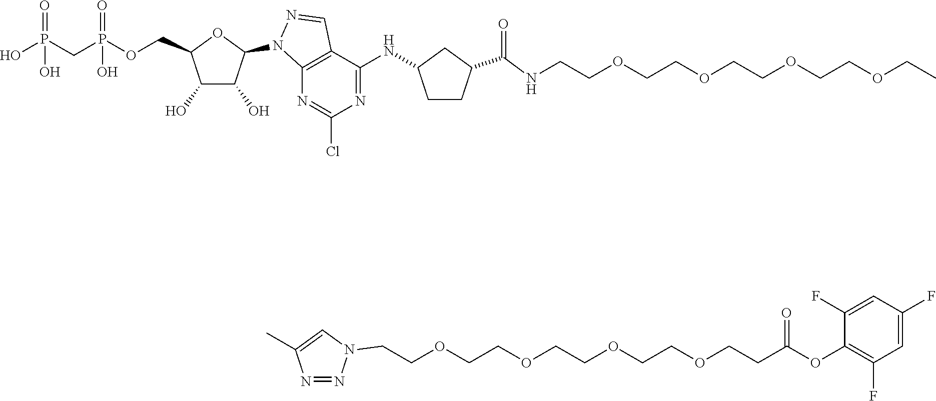 |
| Int-24 | 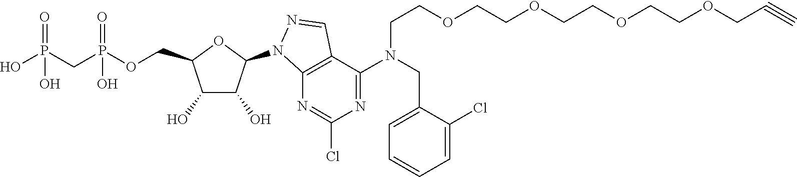 [SMILES: C#CCOCCOCCOCCOCCN(Cc1ccccc1Cl)c1nc(Cl)nc2c1cnn2[C@@H]1O[C@H](COP(=O)(O)CP(=O)(O)O)[C@@H](O)[C@H]1O] |
| Int-25 | 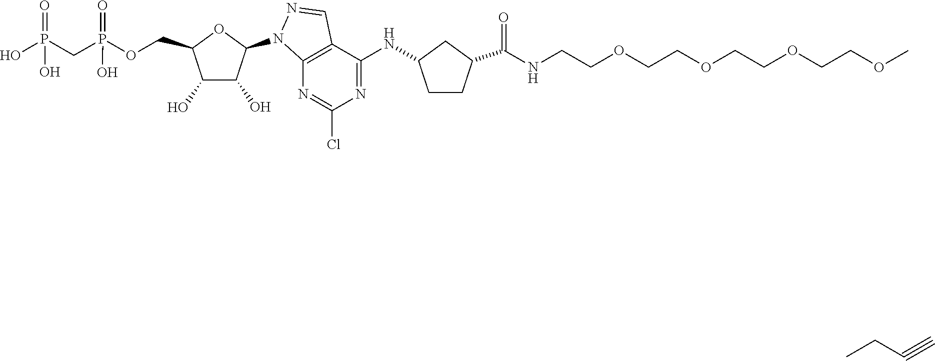 |
| Int-26 | 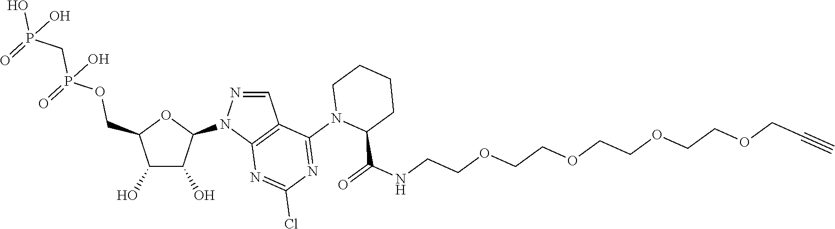 [SMILES: C#CCOCCOCCOCCOCCNC(=O)[C@@H]1CCCCN1c1nc(Cl)nc2c1cnn2[C@@H]1O[C@H](COP(=O)(O)CP(=O)(O)O)[C@@H](O)[C@H]1O] |
| Int-27 | 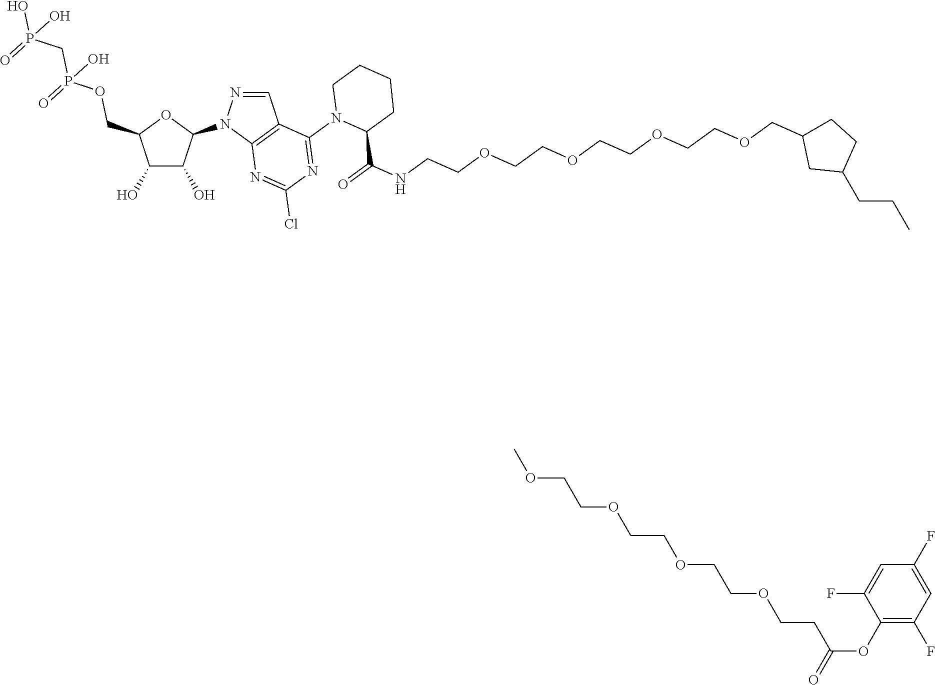 |
| Int-28 | 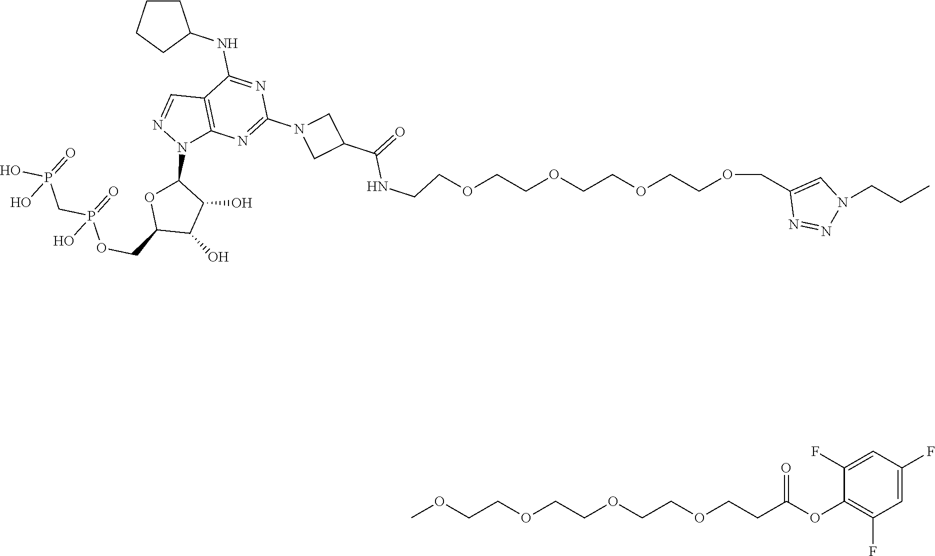 |
| Int-29 | 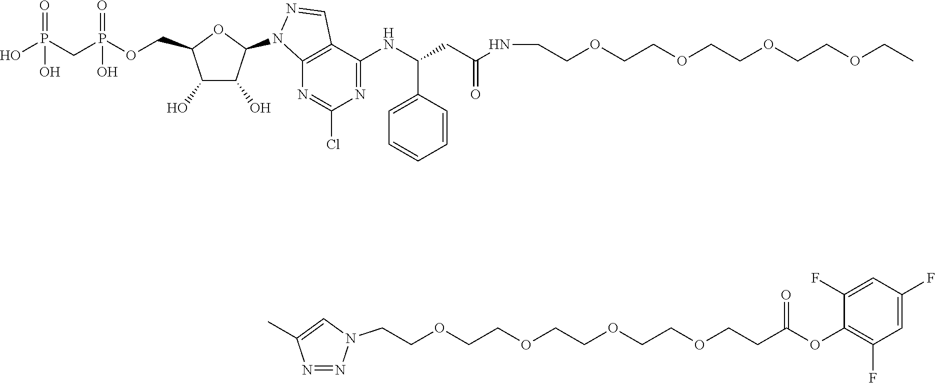 |
| Int-30 | 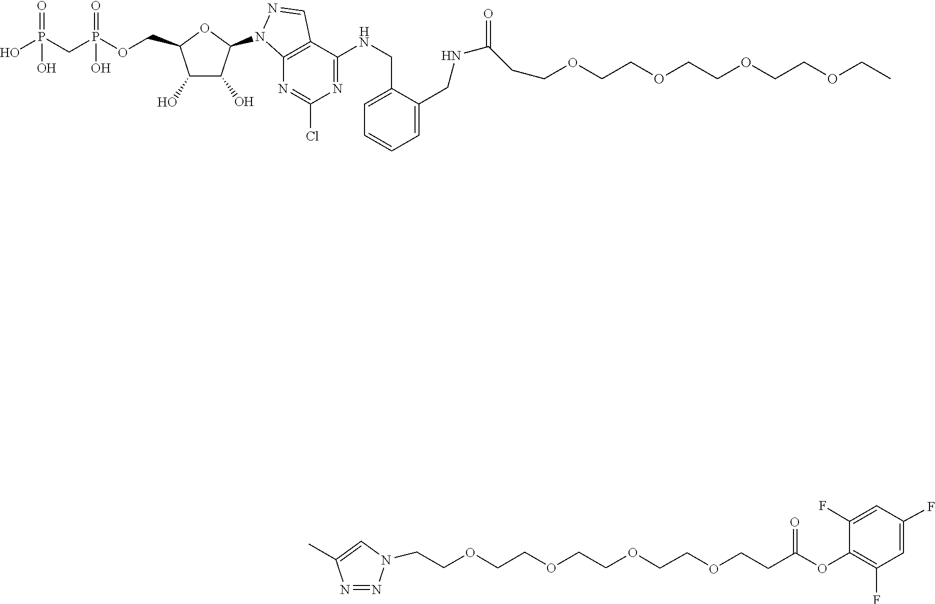 |
| Int-31 | 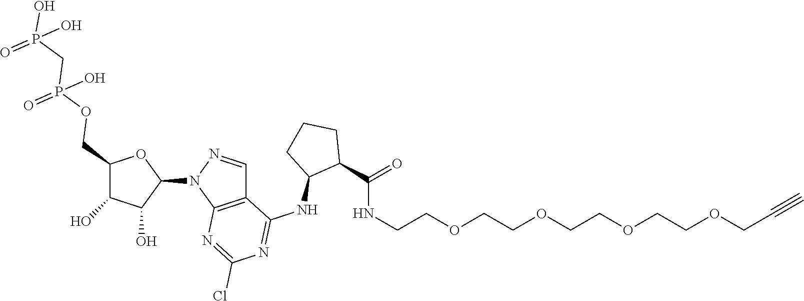 [SMILES: C#CCOCCOCCOCCOCCNC(=O)[C@@H]1CCC[C@@H]1Nc1nc(Cl)nc2c1cnn2[C@@H]1O[C@H](COP(=O)(O)CP(=O)(O)O)[C@@H](O)[C@H]1O] |
| Int-32 | 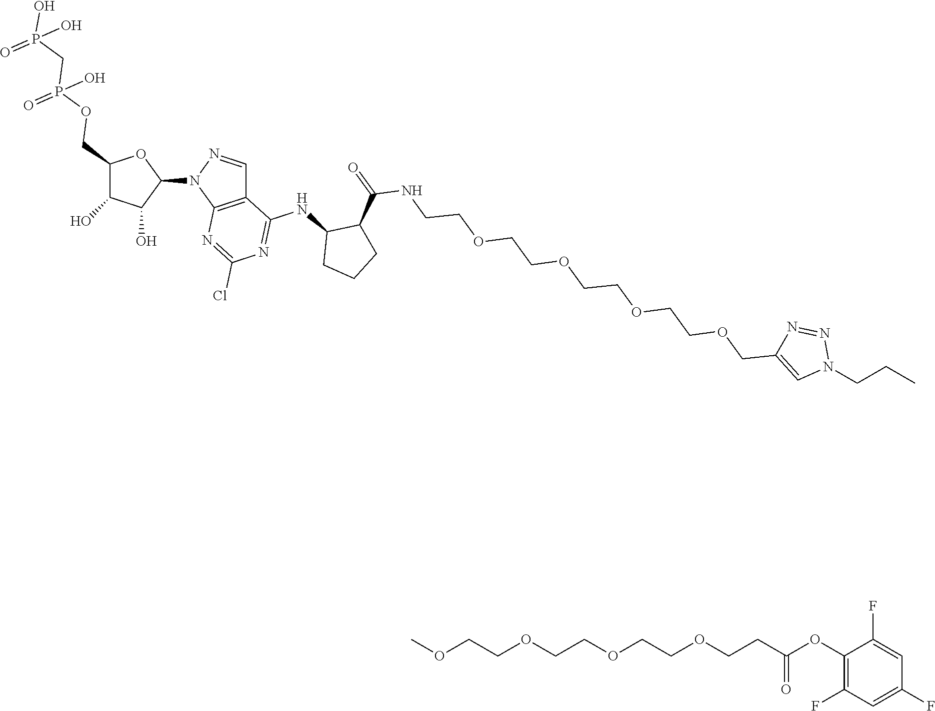 |
| Int-33 | 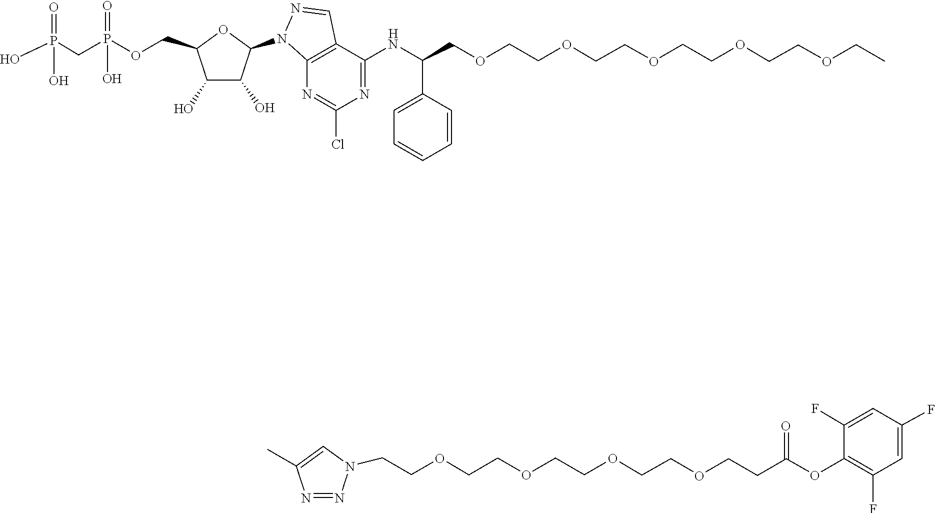 |
| Int-34 | 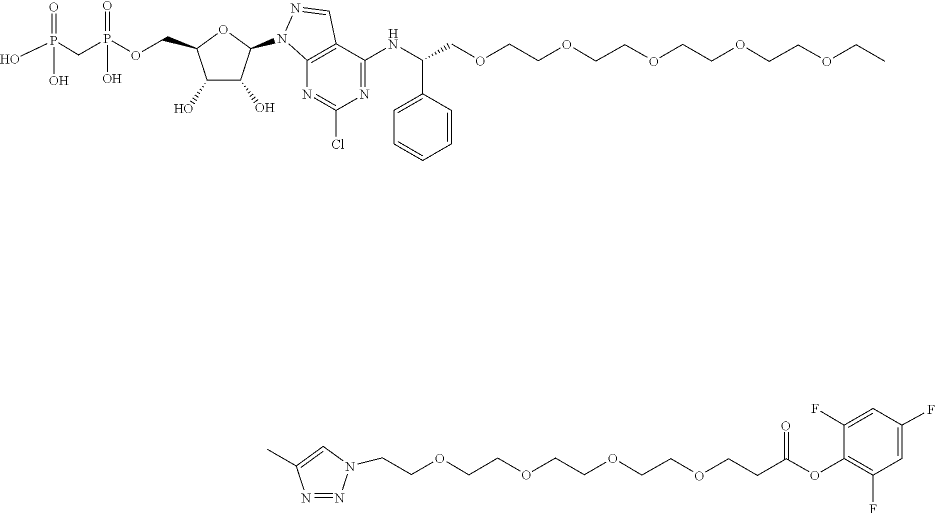 |
| Int-35 | 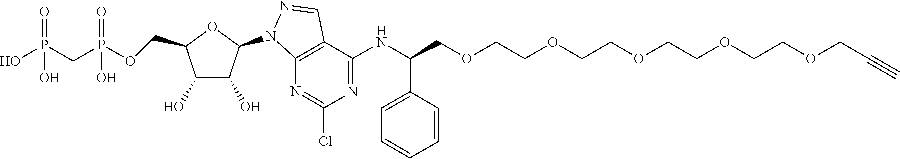 [SMILES: C#CCOCCOCCOCCOCCOC[C@H](Nc1nc(Cl)nc2c1cnn2[C@@H]1O[C@H](COP(=O)(O)CP(=O)(O)O)[C@@H](O)[C@H]1O)c1ccccc1] |
| Int-36 | 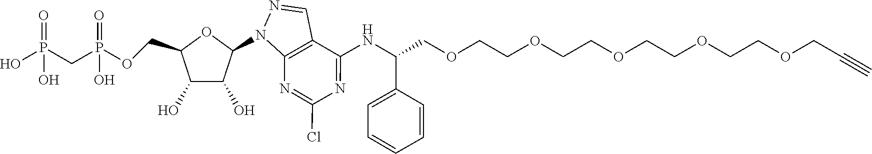 [SMILES: C#CCOCCOCCOCCOCCOC[C@@H](Nc1nc(Cl)nc2c1cnn2[C@@H]1O[C@H](COP(=O)(O)CP(=O)(O)O)[C@@H](O)[C@H]1O)c1ccccc1] |
| Int-37 | 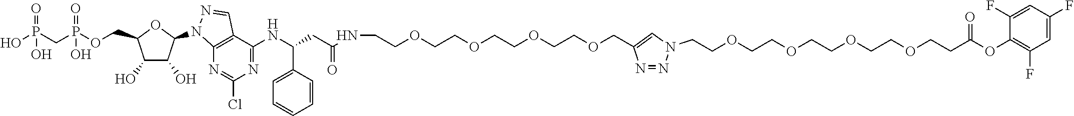 [SMILES: O=C(C[C@@H](Nc1nc(Cl)nc2c1cnn2[C@@H]1O[C@H](COP(=O)(O)CP(=O)(O)O)[C@@H](O)[C@H]1O)c1ccccc1)NCCOCCOCCOCCOCc1cn(CCOCCOCCOCCOCCC(=O)Oc2c(F)cc(F)cc2F)nn1] |
| Int-38 | 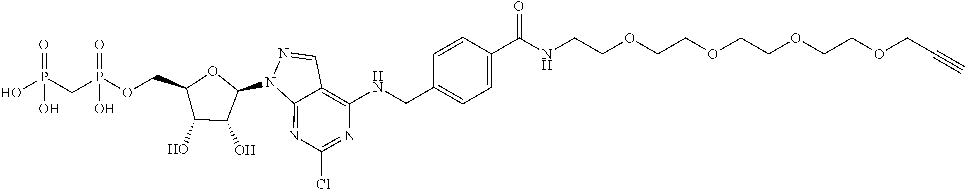 [SMILES: C#CCOCCOCCOCCOCCNC(=O)c1ccc(CNc2nc(Cl)nc3c2cnn3[C@@H]2O[C@H](COP(=O)(O)CP(=O)(O)O)[C@@H](O)[C@H]2O)cc1] |
| Int-39 | 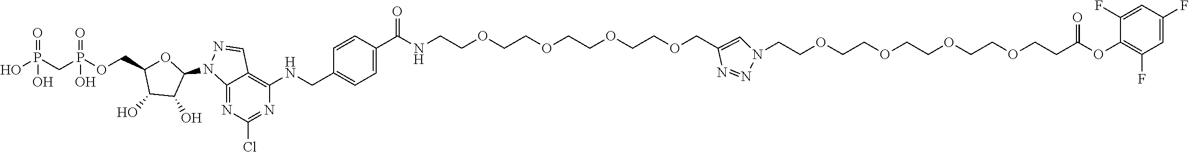 [SMILES: O=C(CCOCCOCCOCCOCCn1cc(COCCOCCOCCOCCNC(=O)c2ccc(CNc3nc(Cl)nc4c3cnn4[C@@H]3O[C@H](COP(=O)(O)CP(=O)(O)O)[C@@H](O)[C@H]3O)cc2)nn1)Oc1c(F)cc(F)cc1F] |
| Int-40 | 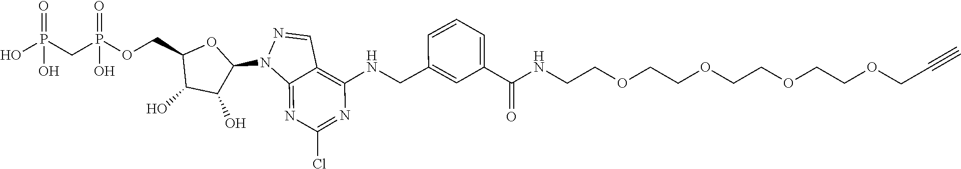 [SMILES: C#CCOCCOCCOCCOCCNC(=O)c1cccc(CNc2nc(Cl)nc3c2cnn3[C@@H]2O[C@H](COP(=O)(O)CP(=O)(O)O)[C@@H](O)[C@H]2O)c1] |
| Int-41 | 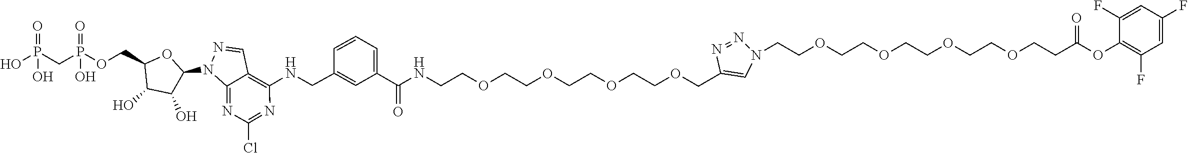 [SMILES: O=C(CCOCCOCCOCCOCCn1cc(COCCOCCOCCOCCNC(=O)c2cccc(CNc3nc(Cl)nc4c3cnn4[C@@H]3O[C@H](COP(=O)(O)CP(=O)(O)O)[C@@H](O)[C@H]3O)c2)nn1)Oc1c(F)cc(F)cc1F] |
| Int-42 | 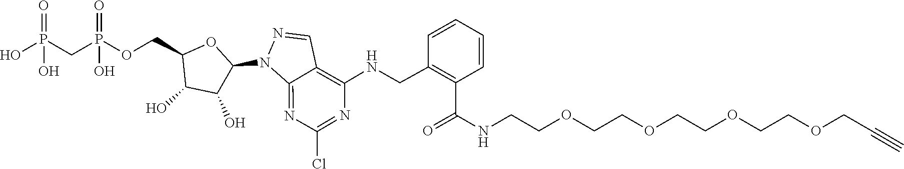 [SMILES: C#CCOCCOCCOCCOCCNC(=O)c1ccccc1CNc1nc(Cl)nc2c1cnn2[C@@H]1O[C@H](COP(=O)(O)CP(=O)(O)O)[C@@H](O)[C@H]1O] |
| Int-43 | 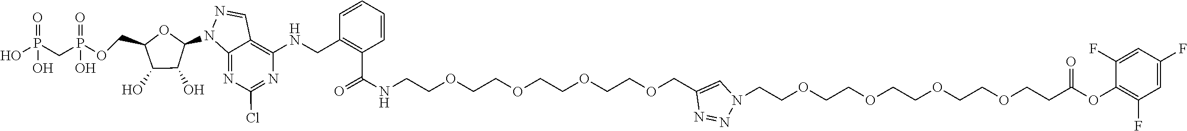 [SMILES: O=C(CCOCCOCCOCCOCCn1cc(COCCOCCOCCOCCNC(=O)c2ccccc2CNc2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-44 | 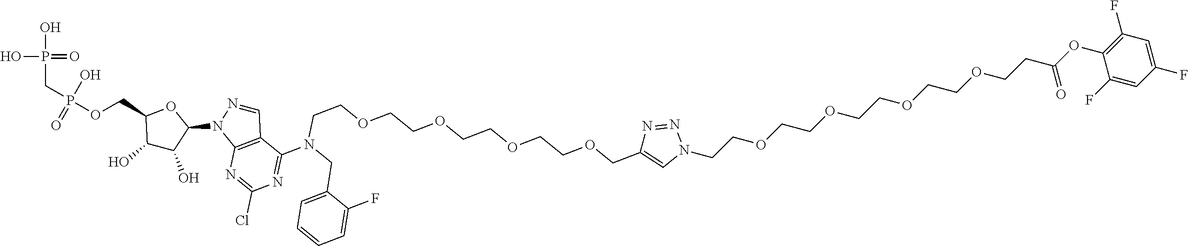 [SMILES: O=C(CCOCCOCCOCCOCCn1cc(COCCOCCOCCOCCN(Cc2ccccc2F)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-45 | 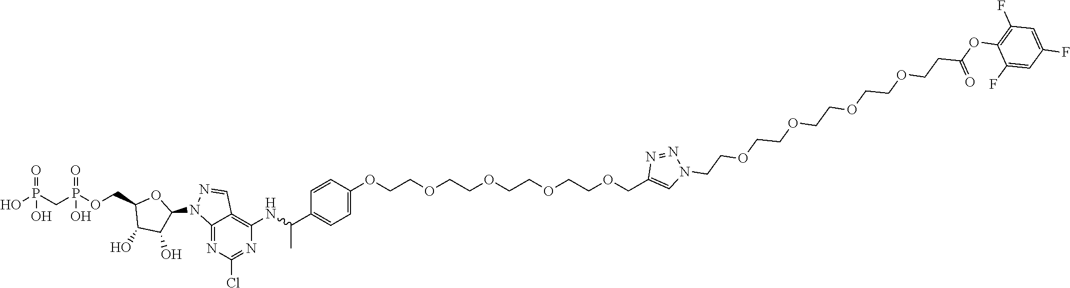 [SMILES: CC(Nc1nc(Cl)nc2c1cnn2[C@@H]1O[C@H](COP(=O)(O)CP(=O)(O)O)[C@@H](O)[C@H]1O)c1ccc(OCCOCCOCCOCCOCc2cn(CCOCCOCCOCCOCCC(=O)Oc3c(F)cc(F)cc3F)nn2)cc1] |
| Int-46 | 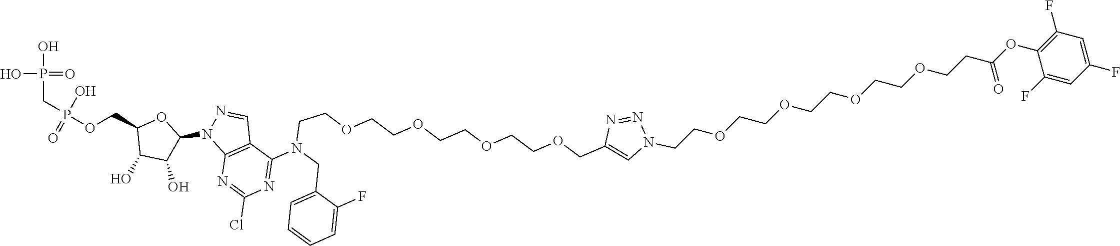 [SMILES: O=C(CCOCCOCCOCCOCCn1cc(COCCOCCOCCOCCN(Cc2ccccc2F)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-47 | 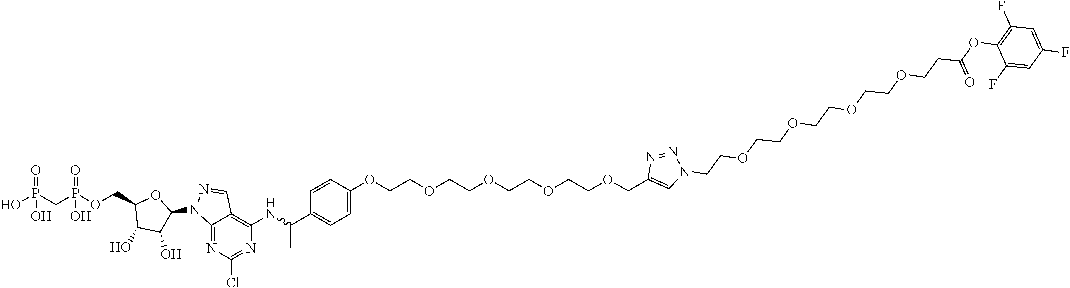 [SMILES: CC(Nc1nc(Cl)nc2c1cnn2[C@@H]1O[C@H](COP(=O)(O)CP(=O)(O)O)[C@@H](O)[C@H]1O)c1ccc(OCCOCCOCCOCCOCc2cn(CCOCCOCCOCCOCCC(=O)Oc3c(F)cc(F)cc3F)nn2)cc1] |
| Int-48 | 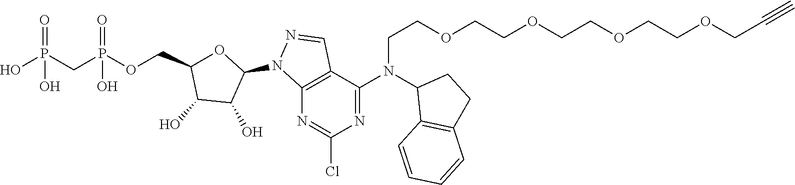 [SMILES: C#CCOCCOCCOCCOCCN(c1nc(Cl)nc2c1cnn2[C@@H]1O[C@H](COP(=O)(O)CP(=O)(O)O)[C@@H](O)[C@H]1O)C1CCc2ccccc21] |
| Int-49 | 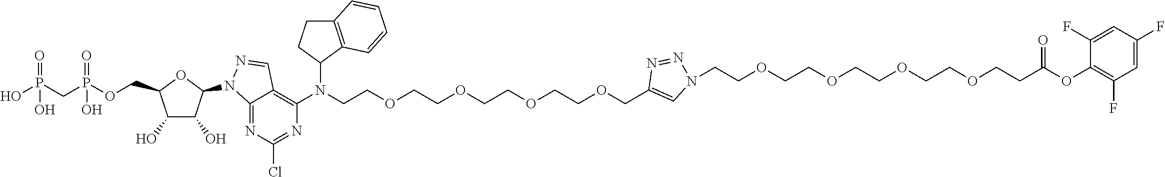 [SMILES: O=C(CCOCCOCCOCCOCCn1cc(COCCOCCOCCOCCN(c2nc(Cl)nc3c2cnn3[C@@H]2O[C@H](COP(=O)(O)CP(=O)(O)O)[C@@H](O)[C@H]2O)C2CCc3ccccc32)nn1)Oc1c(F)cc(F)cc1F] |
| Int-50 | 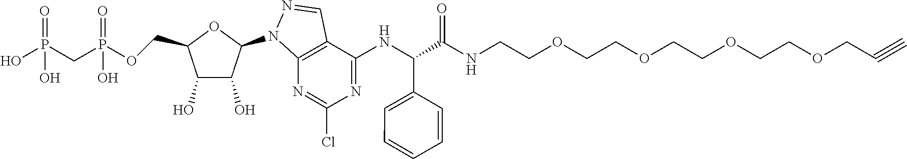 [SMILES: C#CCOCCOCCOCCOCCNC(=O)[C@@H](Nc1nc(Cl)nc2c1cnn2[C@@H]1O[C@H](COP(=O)(O)CP(=O)(O)O)[C@@H](O)[C@H]1O)c1ccccc1] |
| Int-51 | 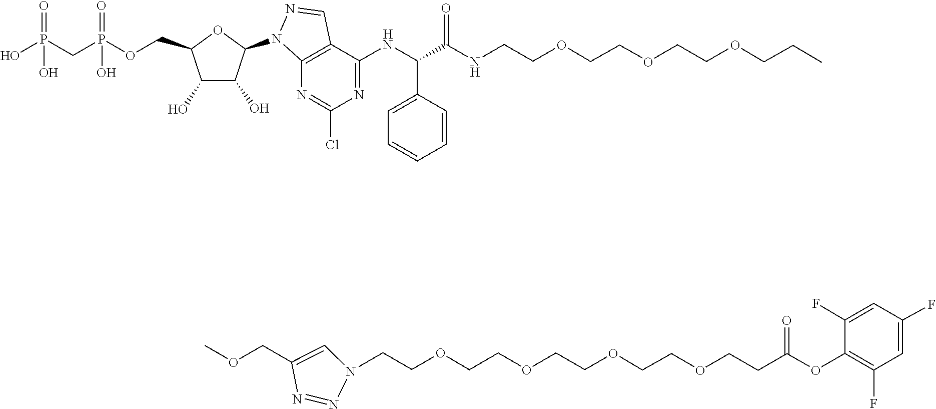 |
| Int-52 | 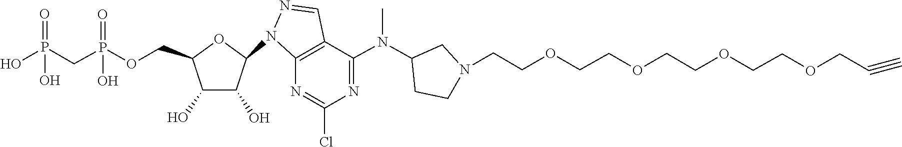 [SMILES: C#CCOCCOCCOCCOCCN1CCC(N(C)c2nc(Cl)nc3c2cnn3[C@@H]2O[C@H](COP(=O)(O)CP(=O)(O)O)[C@@H](O)[C@H]2O)C1] |
| Int-53 | 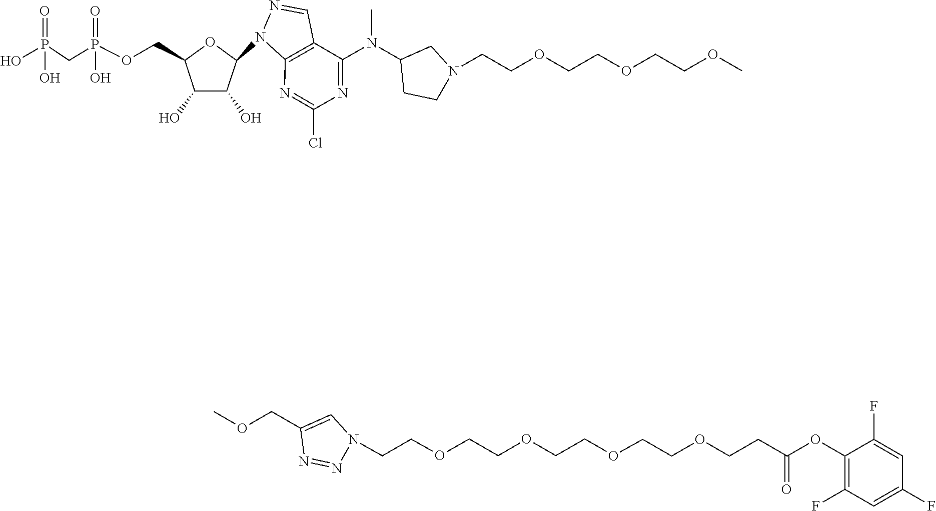 |
| Int-54 | 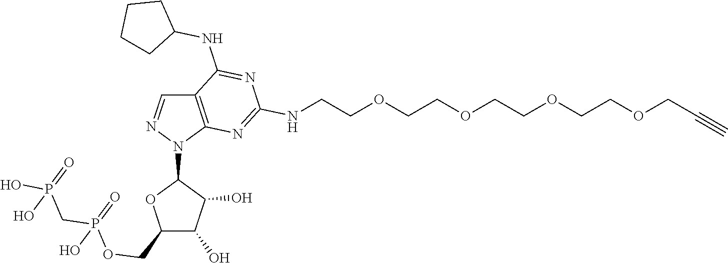 |
| Int-55 | 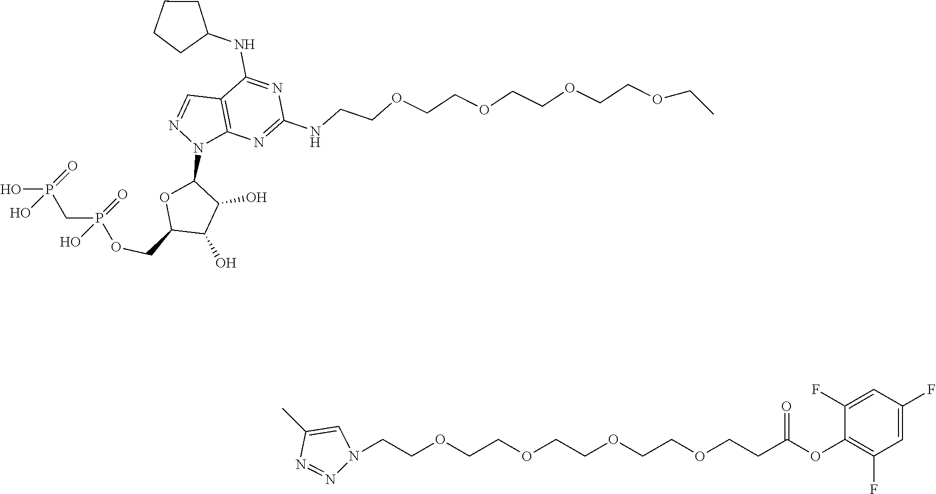 |
| Int-56 | 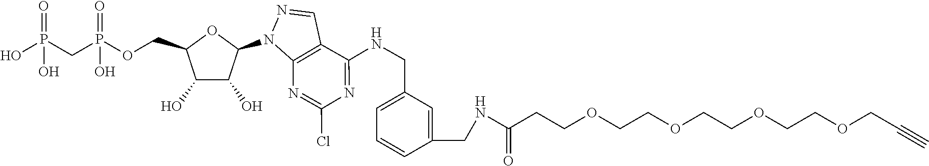 [SMILES: C#CCOCCOCCOCCOCCC(=O)NCc1cccc(CNc2nc(Cl)nc3c2cnn3[C@@H]2O[C@H](COP(=O)(O)CP(=O)(O)O)[C@@H](O)[C@H]2O)c1] |
| Int-57 | 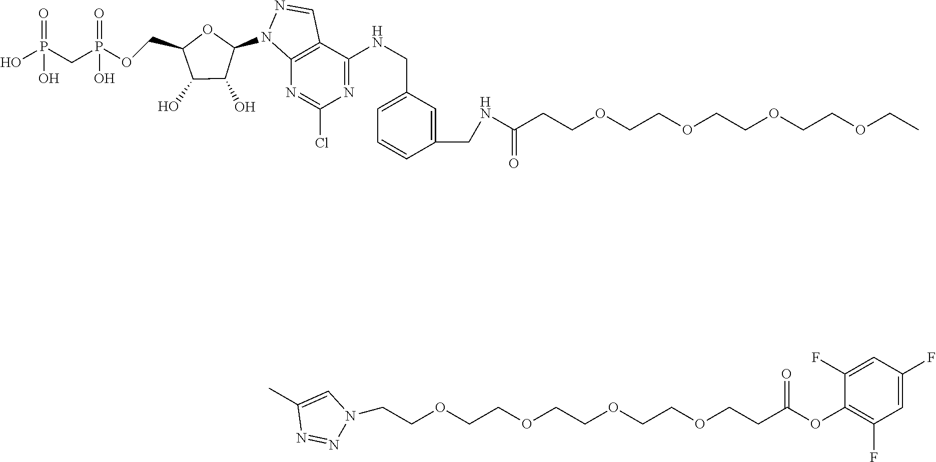 |
| Int-58 | 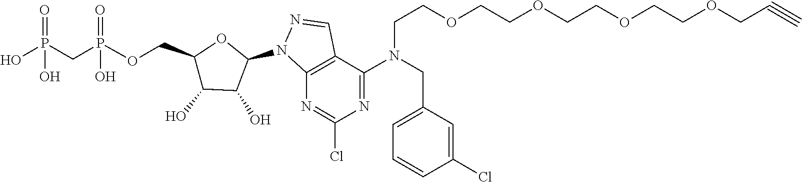 [SMILES: C#CCOCCOCCOCCOCCN(Cc1cccc(Cl)c1)c1nc(Cl)nc2c1cnn2[C@@H]1O[C@H](COP(=O)(O)CP(=O)(O)O)[C@@H](O)[C@H]1O] |
| Int-59 | 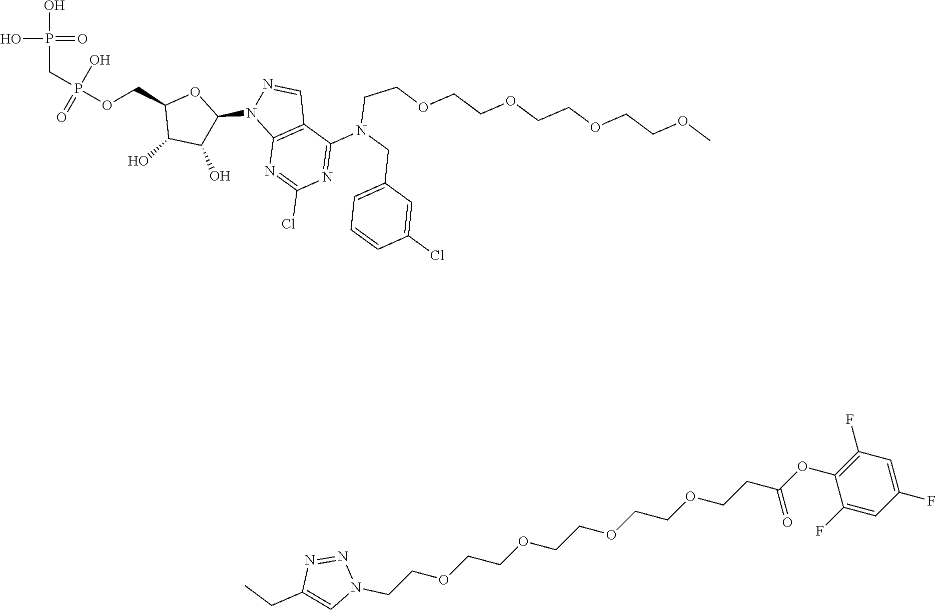 |
| Int-60 | 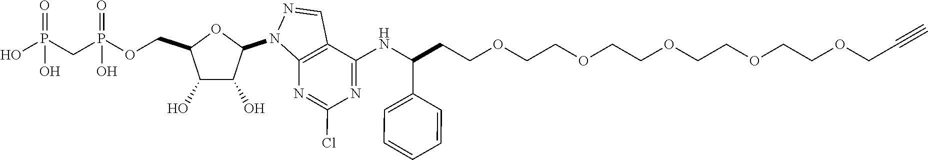 [SMILES: C#CCOCCOCCOCCOCCOCC[C@H](Nc1nc(Cl)nc2c1cnn2[C@@H]1O[C@H](COP(=O)(O)CP(=O)(O)O)[C@@H](O)[C@H]1O)c1ccccc1] |
| Int-61 | 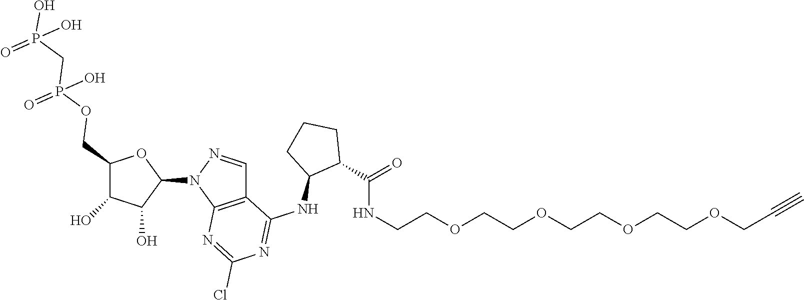 [SMILES: C#CCOCCOCCOCCOCCNC(=O)[C@H]1CCC[C@@H]1Nc1nc(Cl)nc2c1cnn2[C@@H]1O[C@H](COP(=O)(O)CP(=O)(O)O)[C@@H](O)[C@H]1O] |
| Int-62 | 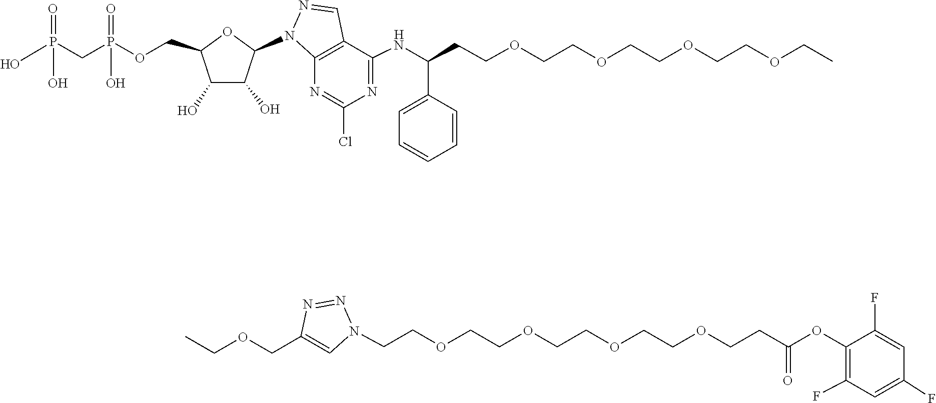 |
| Int-63 | 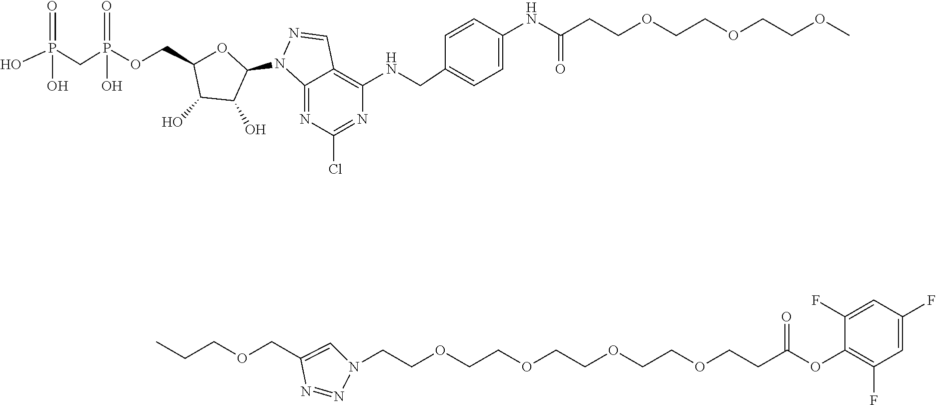 |
| Int-64 | 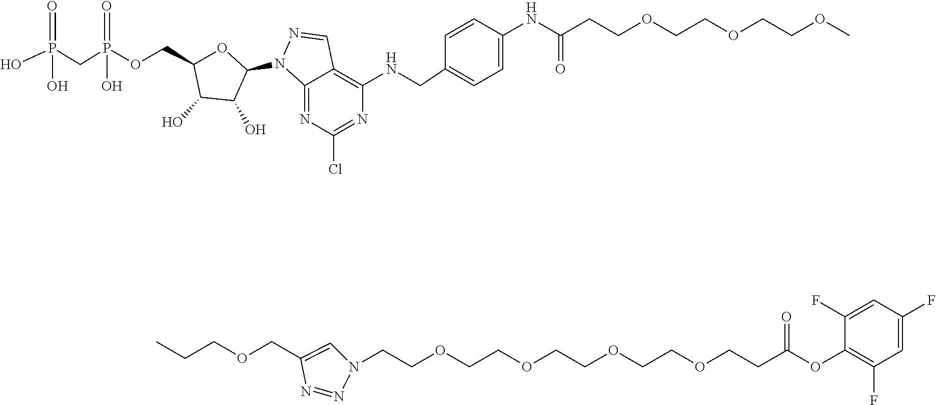 |
| Int-65 | 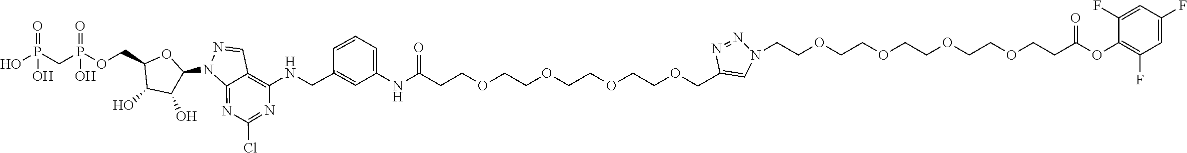 [SMILES: O=C(CCOCCOCCOCCOCc1cn(CCOCCOCCOCCOCCC(=O)Oc2c(F)cc(F)cc2F)nn1)Nc1cccc(CNc2nc(Cl)nc3c2cnn3[C@@H]2O[C@H](COP(=O)(O)CP(=O)(O)O)[C@@H](O)[C@H]2O)c1] |
| Int-66 | 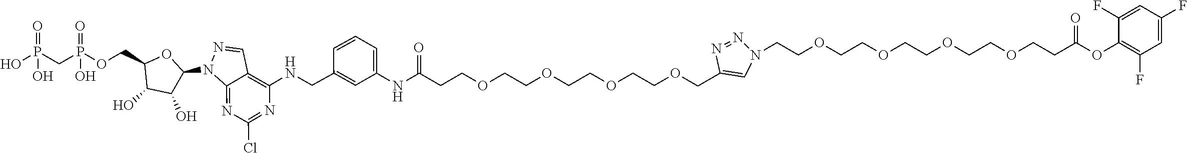 [SMILES: O=C(CCOCCOCCOCCOCc1cn(CCOCCOCCOCCOCCC(=O)Oc2c(F)cc(F)cc2F)nn1)Nc1cccc(CNc2nc(Cl)nc3c2cnn3[C@@H]2O[C@H](COP(=O)(O)CP(=O)(O)O)[C@@H](O)[C@H]2O)c1] |
| Int-67 | 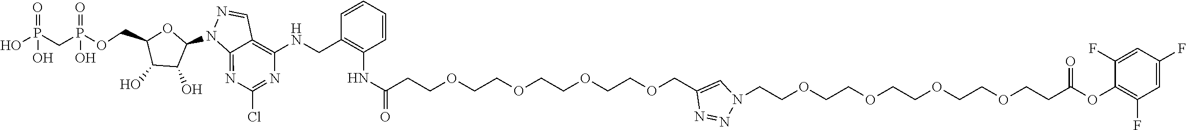 [SMILES: O=C(CCOCCOCCOCCOCc1cn(CCOCCOCCOCCOCCC(=O)Oc2c(F)cc(F)cc2F)nn1)Nc1ccccc1CNc1nc(Cl)nc2c1cnn2[C@@H]1O[C@H](COP(=O)(O)CP(=O)(O)O)[C@@H](O)[C@H]1O] |
| Int-68 | 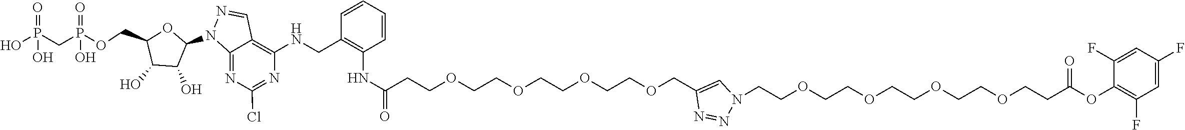 [SMILES: O=C(CCOCCOCCOCCOCc1cn(CCOCCOCCOCCOCCC(=O)Oc2c(F)cc(F)cc2F)nn1)Nc1ccccc1CNc1nc(Cl)nc2c1cnn2[C@@H]1O[C@H](COP(=O)(O)CP(=O)(O)O)[C@@H](O)[C@H]1O] |
| Int-69 | 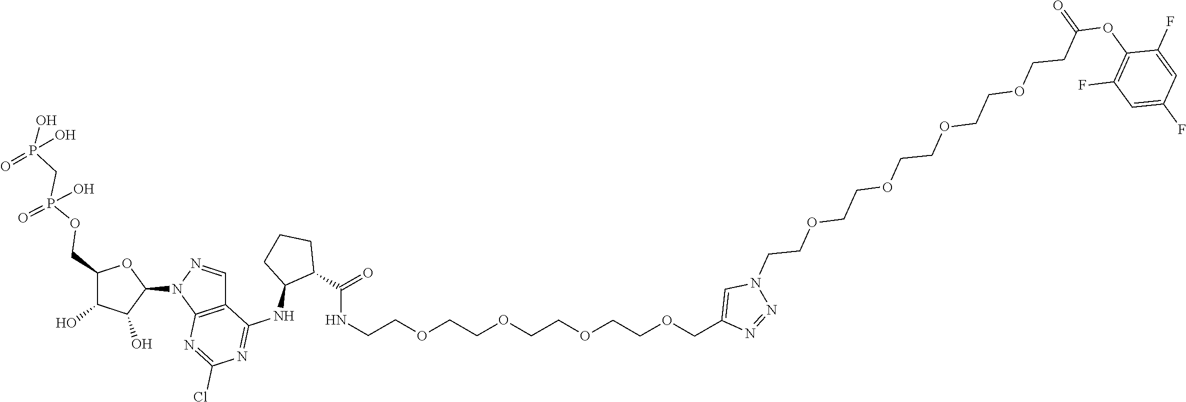 [SMILES: O=C(CCOCCOCCOCCOCCn1cc(COCCOCCOCCOCCNC(=O)[C@H]2CCC[C@@H]2Nc2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-70 | 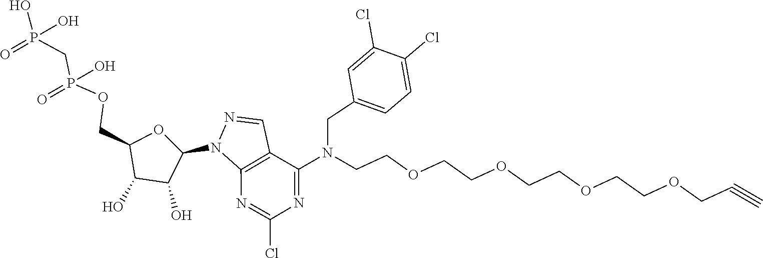 [SMILES: C#CCOCCOCCOCCOCCN(Cc1ccc(Cl)c(Cl)c1)c1nc(Cl)nc2c1cnn2[C@@H]1O[C@H](COP(=O)(O)CP(=O)(O)O)[C@@H](O)[C@H]1O] |
| Int-71 | 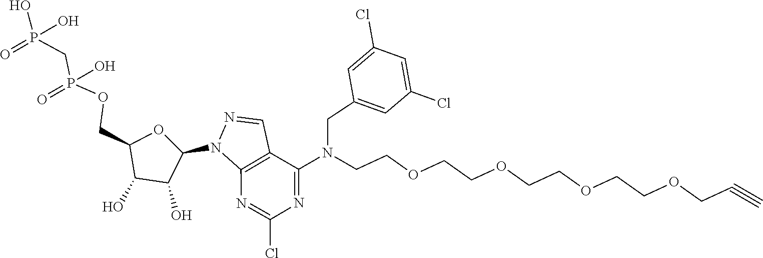 [SMILES: C#CCOCCOCCOCCOCCN(Cc1cc(Cl)cc(Cl)c1)c1nc(Cl)nc2c1cnn2[C@@H]1O[C@H](COP(=O)(O)CP(=O)(O)O)[C@@H](O)[C@H]1O] |
| Int-72 | 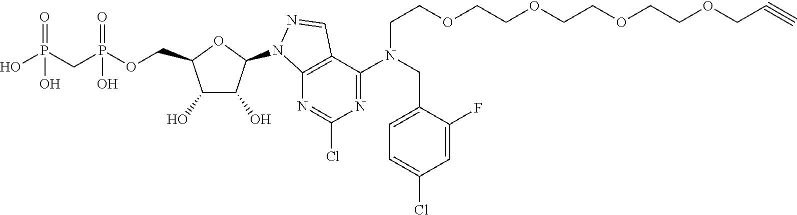 [SMILES: C#CCOCCOCCOCCOCCN(Cc1ccc(Cl)cc1F)c1nc(Cl)nc2c1cnn2[C@@H]1O[C@H](COP(=O)(O)CP(=O)(O)O)[C@@H](O)[C@H]1O] |
| Int-73 | 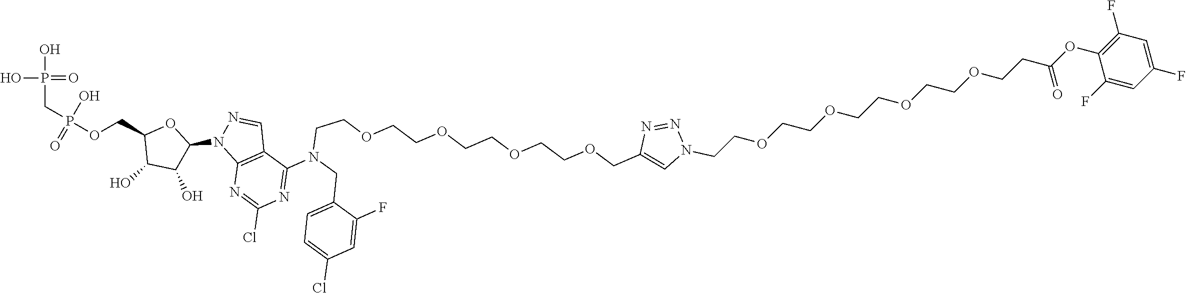 [SMILES: O=C(CCOCCOCCOCCOCCn1cc(COCCOCCOCCOCCN(Cc2ccc(Cl)cc2F)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-74 | 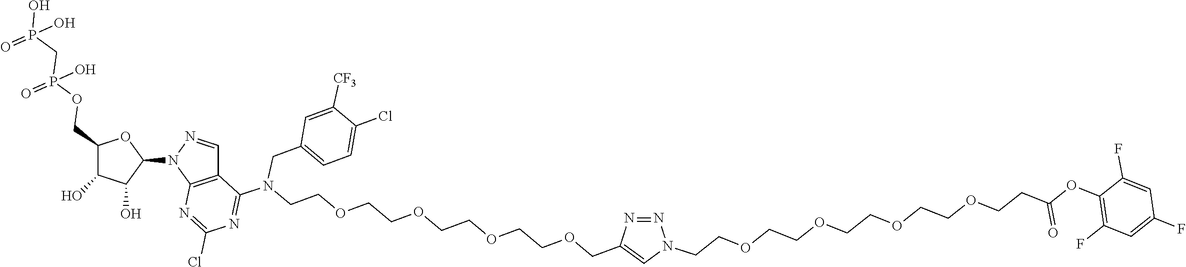 [SMILES: O=C(CCOCCOCCOCCOCCn1cc(COCCOCCOCCOCCN(Cc2ccc(Cl)c(C(F)(F)F)c2)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-75 | 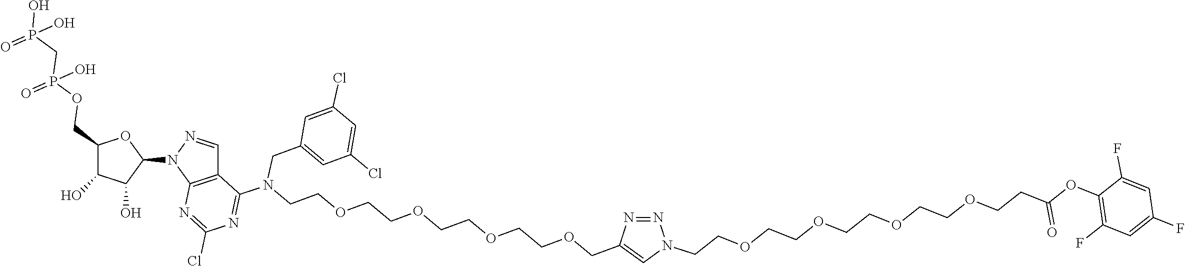 [SMILES: O=C(CCOCCOCCOCCOCCn1cc(COCCOCCOCCOCCN(Cc2cc(Cl)cc(Cl)c2)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-76 | 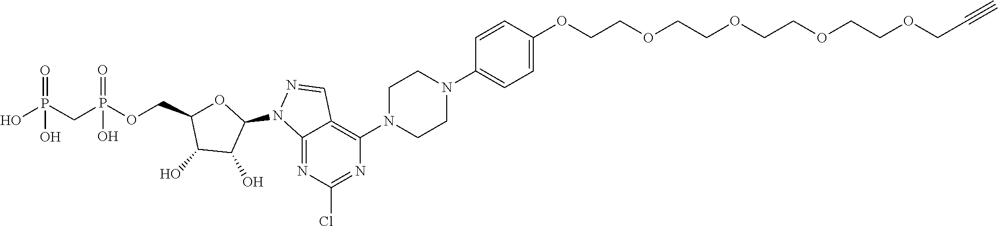 [SMILES: C#CCOCCOCCOCCOCCOc1ccc(N2CCN(c3nc(Cl)nc4c3cnn4[C@@H]3O[C@H](COP(=O)(O)CP(=O)(O)O)[C@@H](O)[C@H]3O)CC2)cc1] |
| Int-77 | 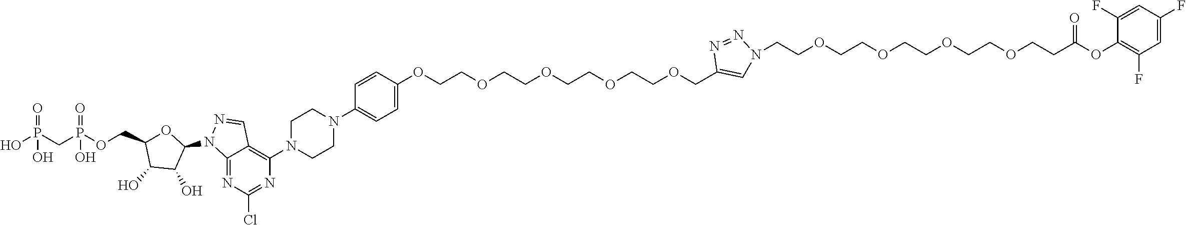 [SMILES: O=C(CCOCCOCCOCCOCCn1cc(COCCOCCOCCOCCOc2ccc(N3CCN(c4nc(Cl)nc5c4cnn5[C@@H]4O[C@H](COP(=O)(O)CP(=O)(O)O)[C@@H](O)[C@H]4O)CC3)cc2)nn1)Oc1c(F)cc(F)cc1F] |
| Int-78 | 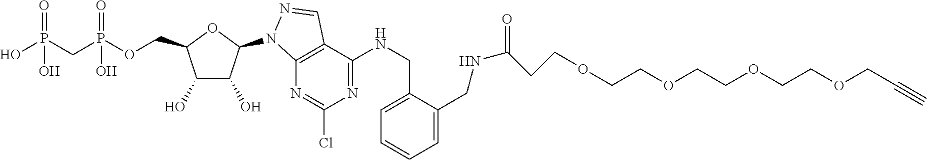 [SMILES: C#CCOCCOCCOCCOCCC(=O)NCc1ccccc1CNc1nc(Cl)nc2c1cnn2[C@@H]1O[C@H](COP(=O)(O)CP(=O)(O)O)[C@@H](O)[C@H]1O] |
| Int-79 | 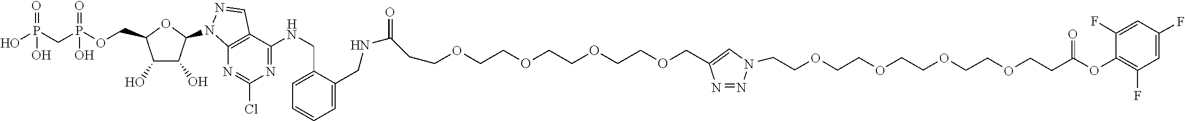 [SMILES: O=C(CCOCCOCCOCCOCc1cn(CCOCCOCCOCCOCCC(=O)Oc2c(F)cc(F)cc2F)nn1)NCc1ccccc1CNc1nc(Cl)nc2c1cnn2[C@@H]1O[C@H](COP(=O)(O)CP(=O)(O)O)[C@@H](O)[C@H]1O] |
| Int-80 | 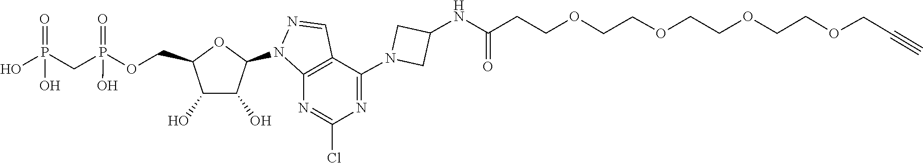 [SMILES: C#CCOCCOCCOCCOCCC(=O)NC1CN(c2nc(Cl)nc3c2cnn3[C@@H]2O[C@H](COP(=O)(O)CP(=O)(O)O)[C@@H](O)[C@H]2O)C1] |
| Int-81 | 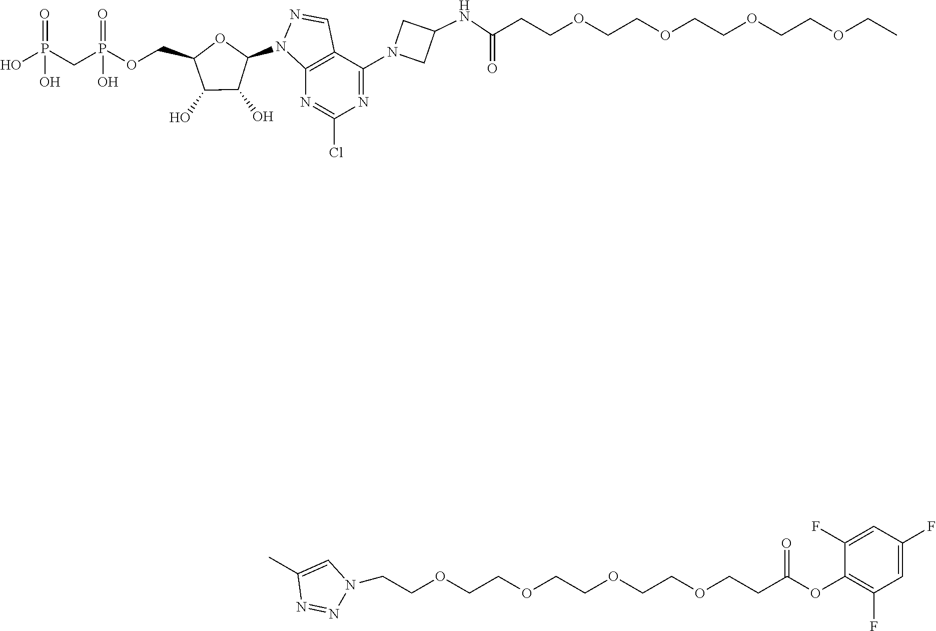 |
| Int-82 | 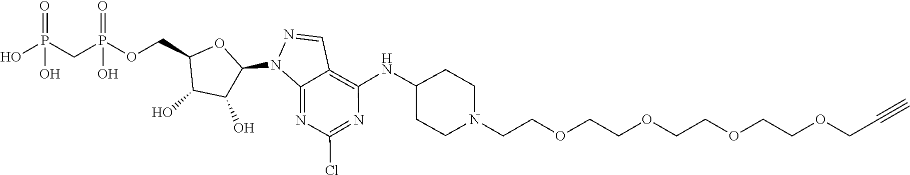 [SMILES: C#CCOCCOCCOCCOCCN1CCC(Nc2nc(Cl)nc3c2cnn3[C@@H]2O[C@H](COP(=O)(O)CP(=O)(O)O)[C@@H](O)[C@H]2O)CC1] |
| Int-83 | 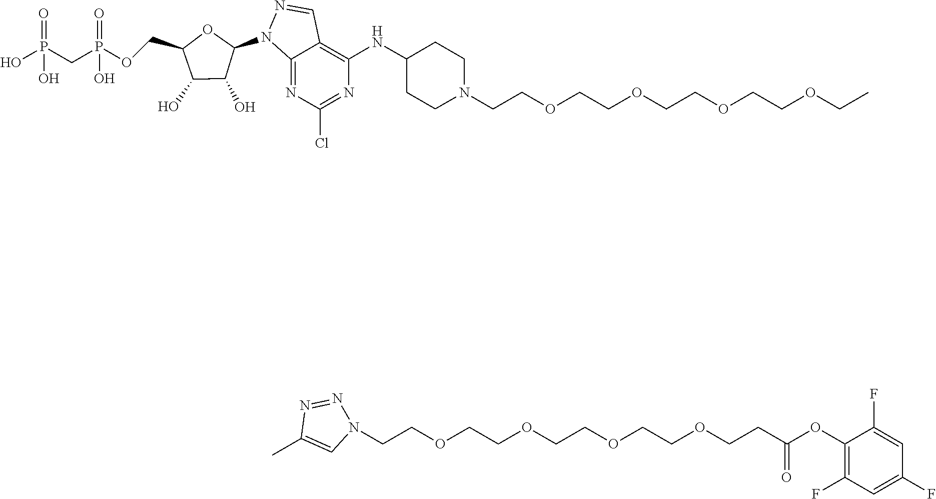 |
| Int-84 | 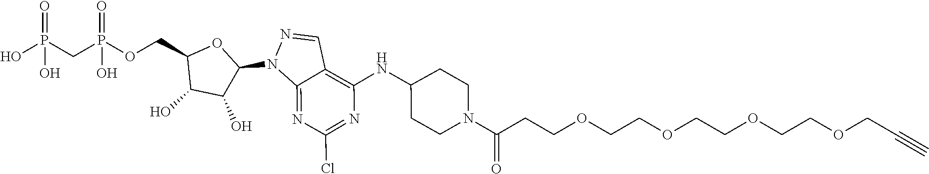 [SMILES: C#CCOCCOCCOCCOCCC(=O)N1CCC(Nc2nc(Cl)nc3c2cnn3[C@@H]2O[C@H](COP(=O)(O)CP(=O)(O)O)[C@@H](O)[C@H]2O)CC1] |
| Int-85 | 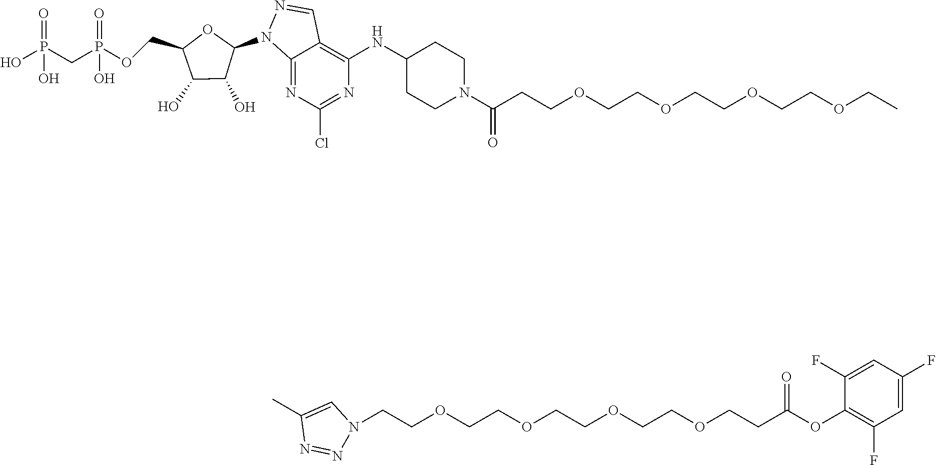 |
| Int-86 | 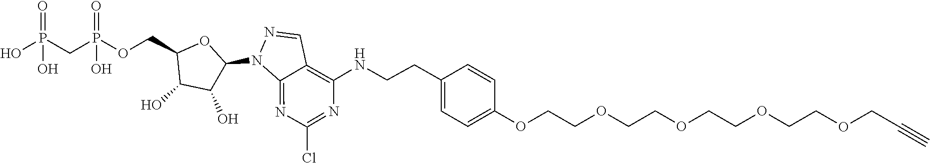 [SMILES: C#CCOCCOCCOCCOCCOc1ccc(CCNc2nc(Cl)nc3c2cnn3[C@@H]2O[C@H](COP(=O)(O)CP(=O)(O)O)[C@@H](O)[C@H]2O)cc1] |
| Int-87 | 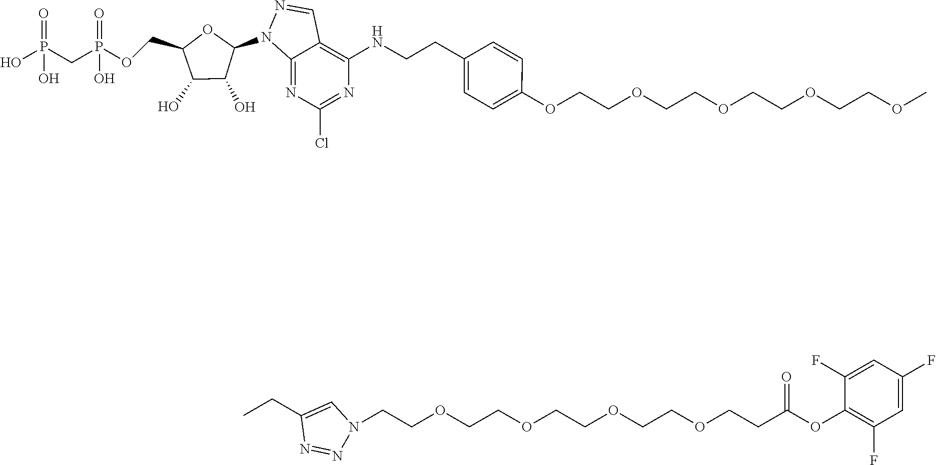 |
| Int-88 | 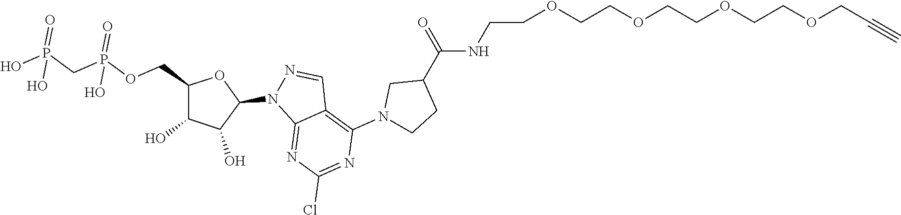 [SMILES: C#CCOCCOCCOCCOCCNC(=O)C1CCN(c2nc(Cl)nc3c2cnn3[C@@H]2O[C@H](COP(=O)(O)CP(=O)(O)O)[C@@H](O)[C@H]2O)C1] |
| Int-89 | 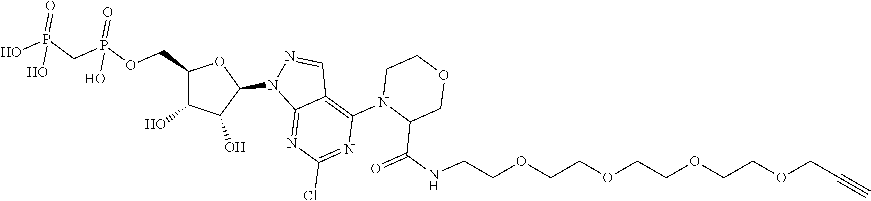 [SMILES: C#CCOCCOCCOCCOCCNC(=O)C1COCCN1c1nc(Cl)nc2c1cnn2[C@@H]1O[C@H](COP(=O)(O)CP(=O)(O)O)[C@@H](O)[C@H]1O] |
| Int-90 | 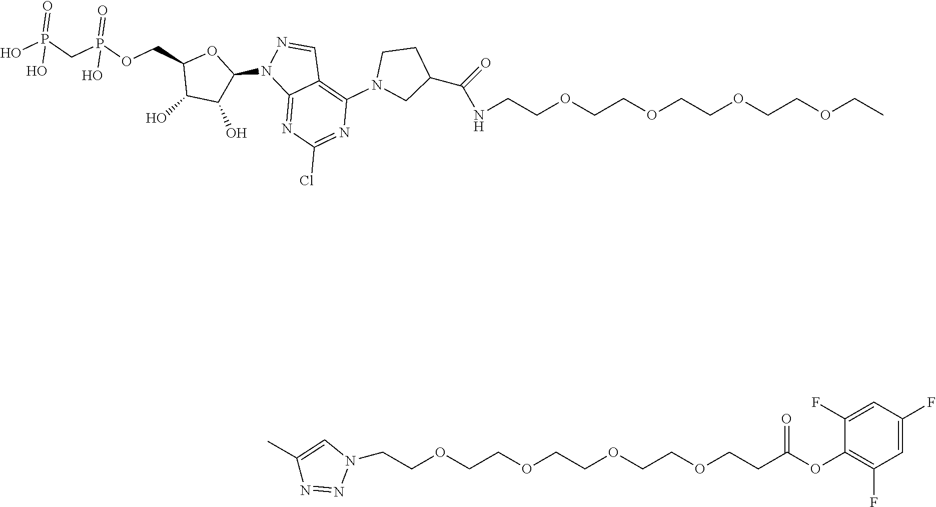 |
| Int-91 | 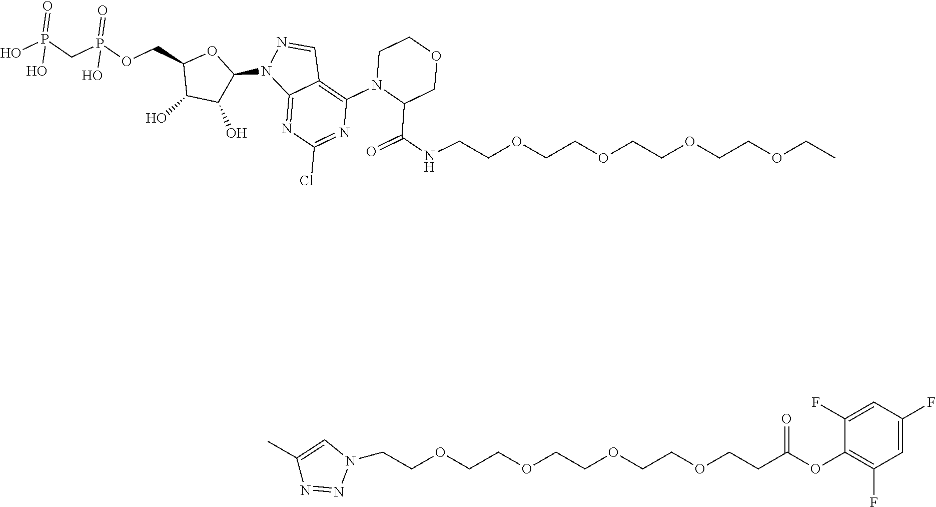 |
| Int-92 | 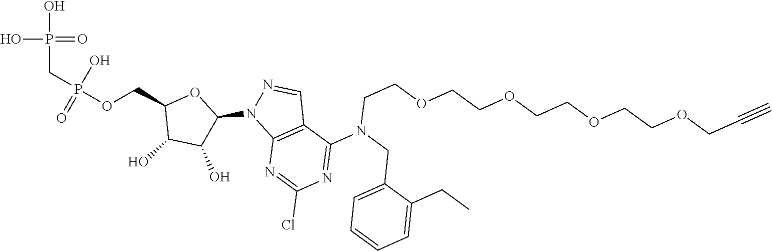 [SMILES: C#CCOCCOCCOCCOCCN(Cc1ccccc1CC)c1nc(Cl)nc2c1cnn2[C@@H]1O[C@H](COP(=O)(O)CP(=O)(O)O)[C@@H](O)[C@H]1O] |
| Int-93 | 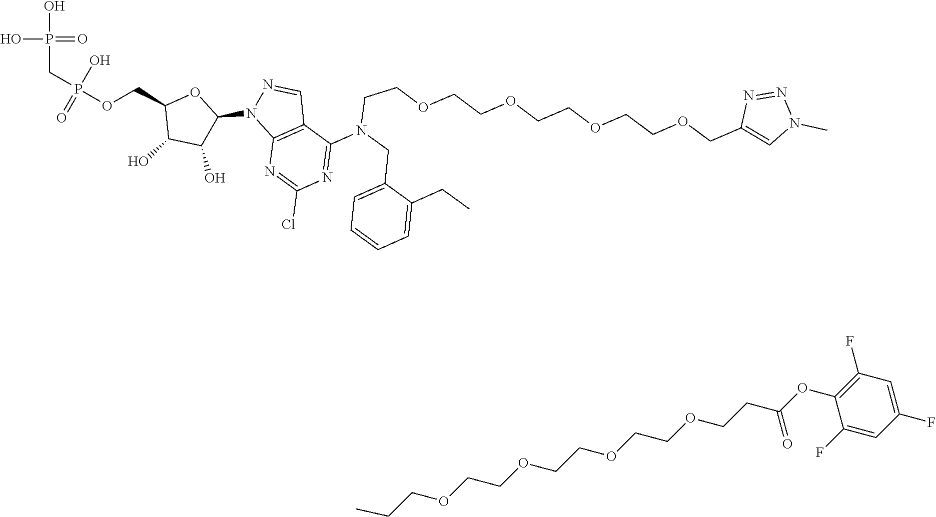 |
| Int-94 | 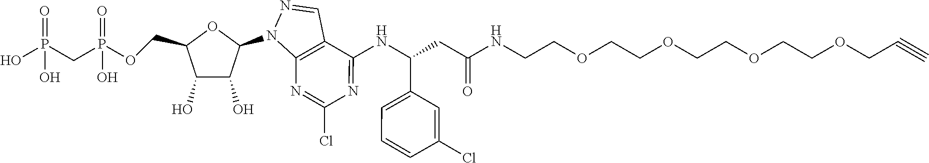 [SMILES: C#CCOCCOCCOCCOCCNC(=O)C[C@@H](Nc1nc(Cl)nc2c1cnn2[C@@H]1O[C@H](COP(=O)(O)CP(=O)(O)O)[C@@H](O)[C@H]1O)c1cccc(Cl)c1] |
| Int-95 | 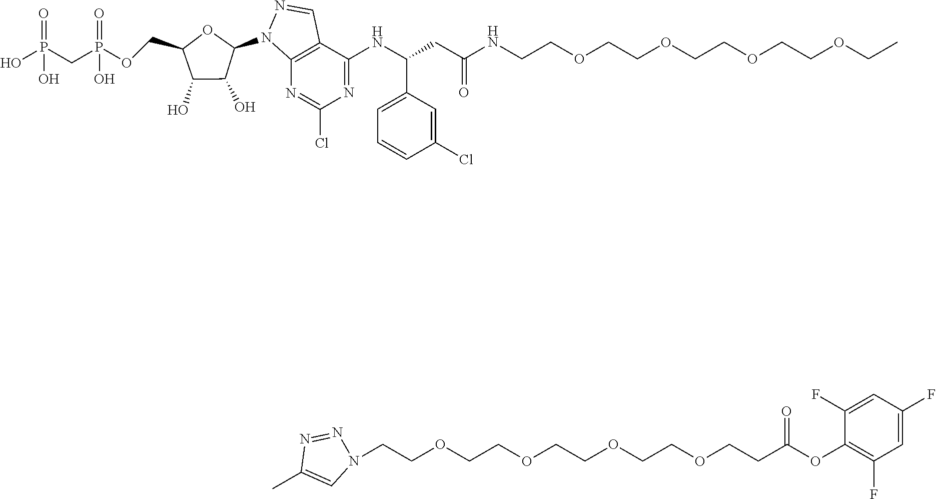 |
| Int-96 | 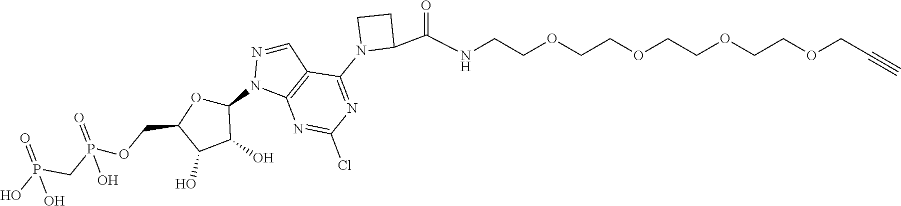 [SMILES: C#CCOCCOCCOCCOCCNC(=O)C1CCN1c1nc(Cl)nc2c1cnn2[C@@H]1O[C@H](COP(=O)(O)CP(=O)(O)O)[C@@H](O)[C@H]1O] |
| Int-97 | 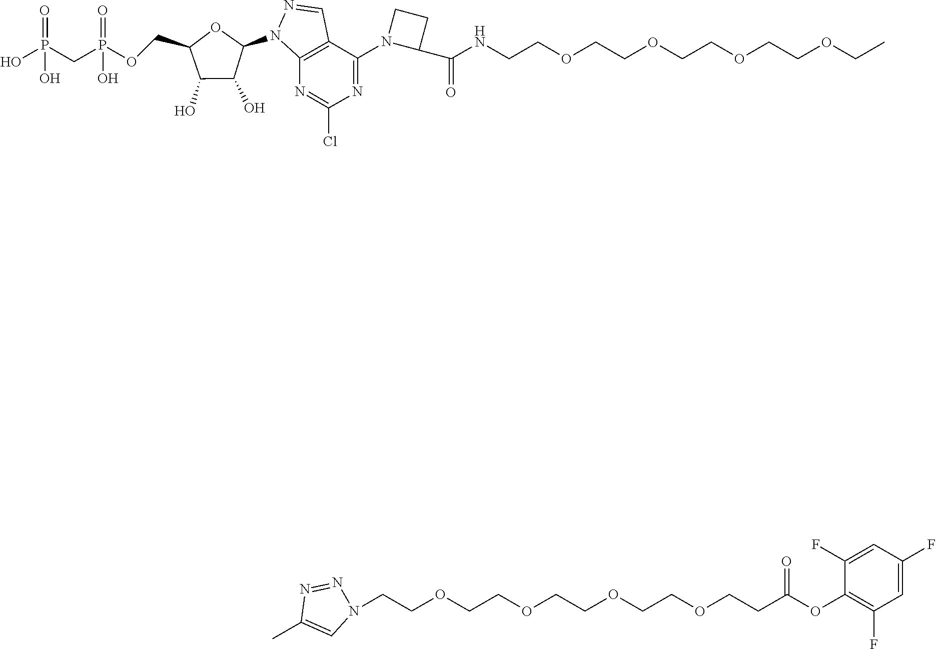 |
| Int-98 | 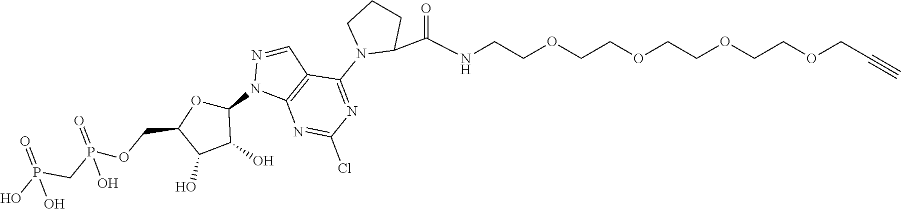 [SMILES: C#CCOCCOCCOCCOCCNC(=O)C1CCCN1c1nc(Cl)nc2c1cnn2[C@@H]1O[C@H](COP(=O)(O)CP(=O)(O)O)[C@@H](O)[C@H]1O] |
| Int-99 | 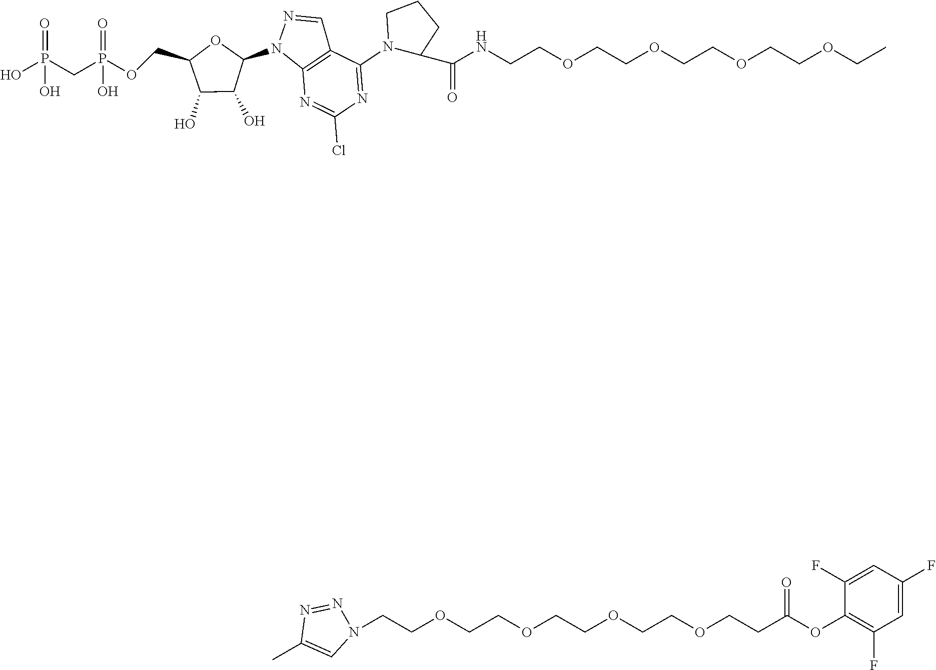 |
| Int-100 | 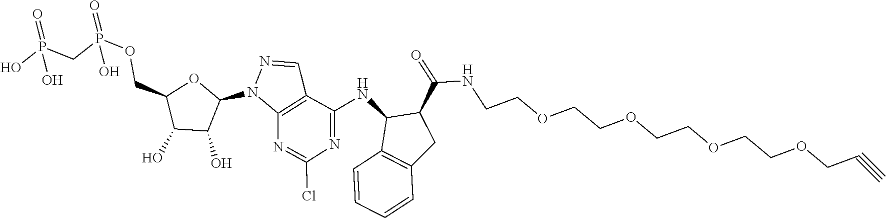 [SMILES: C#CCOCCOCCOCCOCCNC(=O)[C@H]1Cc2ccccc2[C@H]1Nc1nc(Cl)nc2c1cnn2[C@@H]1O[C@H](COP(=O)(O)CP(=O)(O)O)[C@@H](O)[C@H]1O] |
| Int-101 | 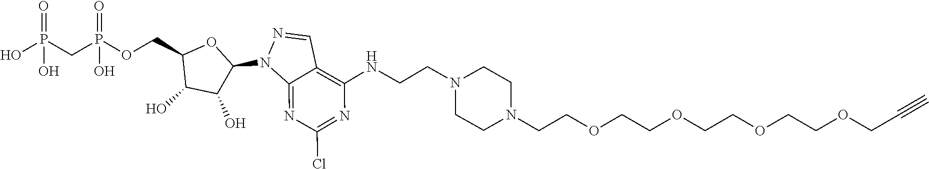 [SMILES: C#CCOCCOCCOCCOCCN1CCN(CCNc2nc(Cl)nc3c2cnn3[C@@H]2O[C@H](COP(=O)(O)CP(=O)(O)O)[C@@H](O)[C@H]2O)CC1] |
| Int-102 | 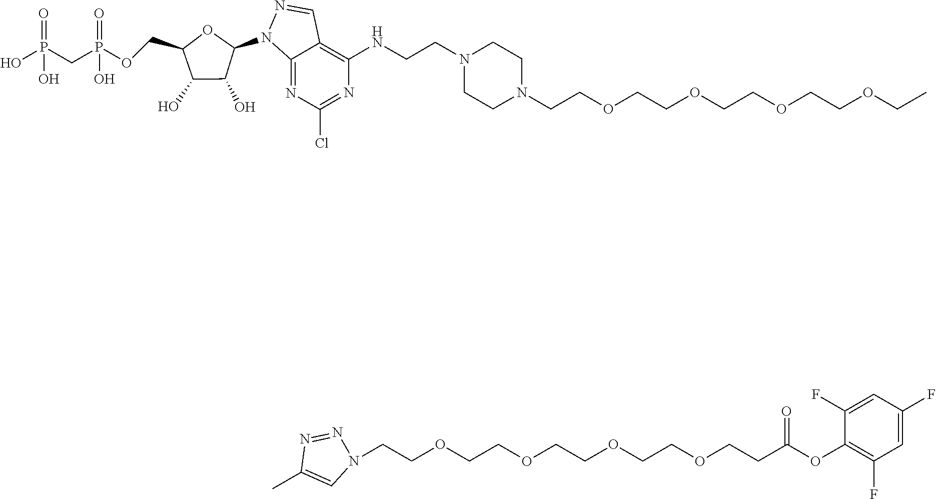 |
| Int-103 | 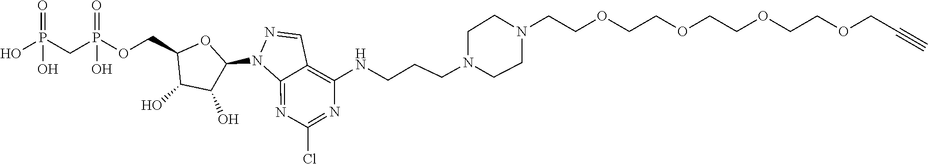 [SMILES: C#CCOCCOCCOCCOCCN1CCN(CCCNc2nc(Cl)nc3c2cnn3[C@@H]2O[C@H](COP(=O)(O)CP(=O)(O)O)[C@@H](O)[C@H]2O)CC1] |
| Int-104 | 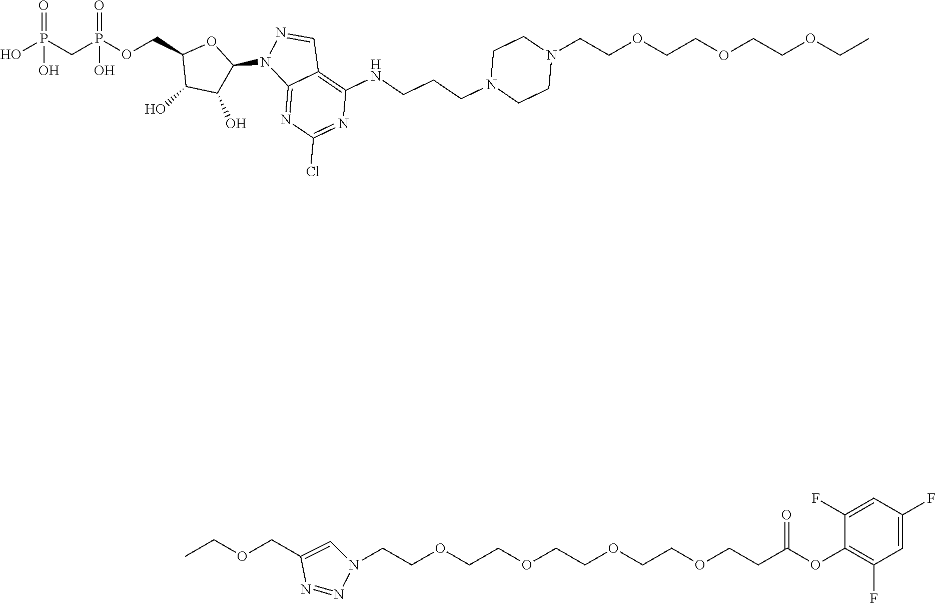 |
| Int-105 | 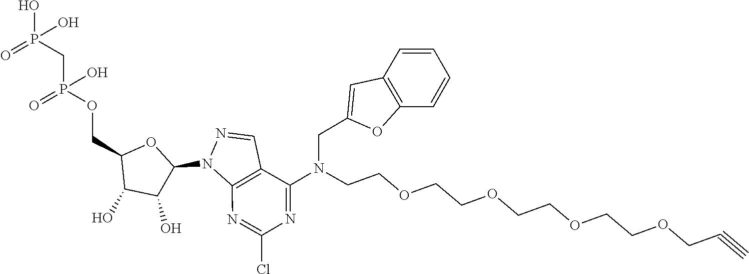 [SMILES: C#CCOCCOCCOCCOCCN(Cc1cc2ccccc2o1)c1nc(Cl)nc2c1cnn2[C@@H]1O[C@H](COP(=O)(O)CP(=O)(O)O)[C@@H](O)[C@H]1O] |
| Int-106 | 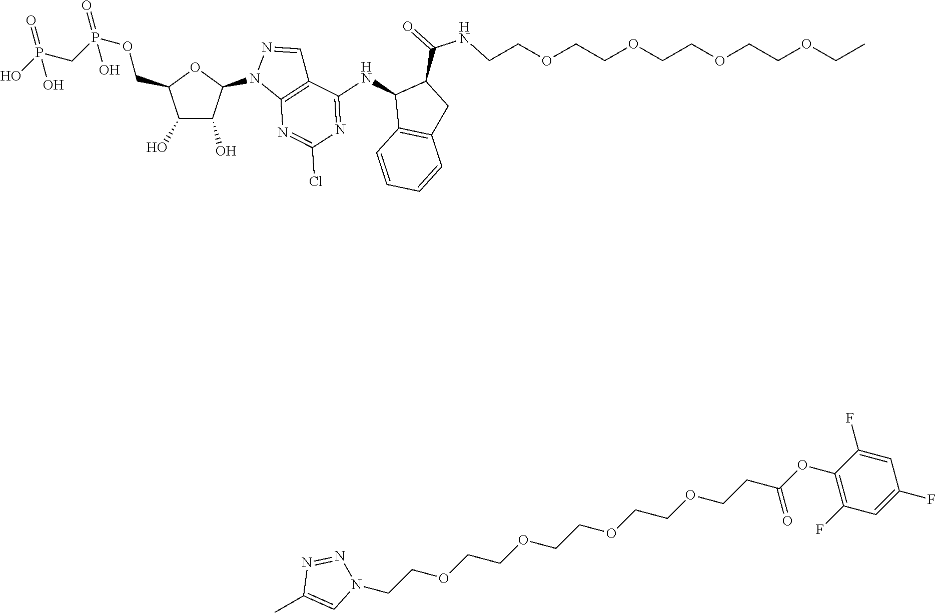 |
| Int-107 | 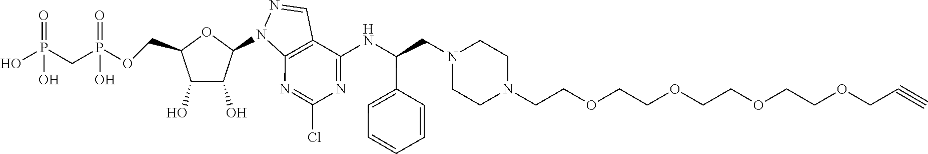 [SMILES: C#CCOCCOCCOCCOCCN1CCN(C[C@H](Nc2nc(Cl)nc3c2cnn3[C@@H]2O[C@H](COP(=O)(O)CP(=O)(O)O)[C@@H](O)[C@H]2O)c2ccccc2)CC1] |
| Int-108 | 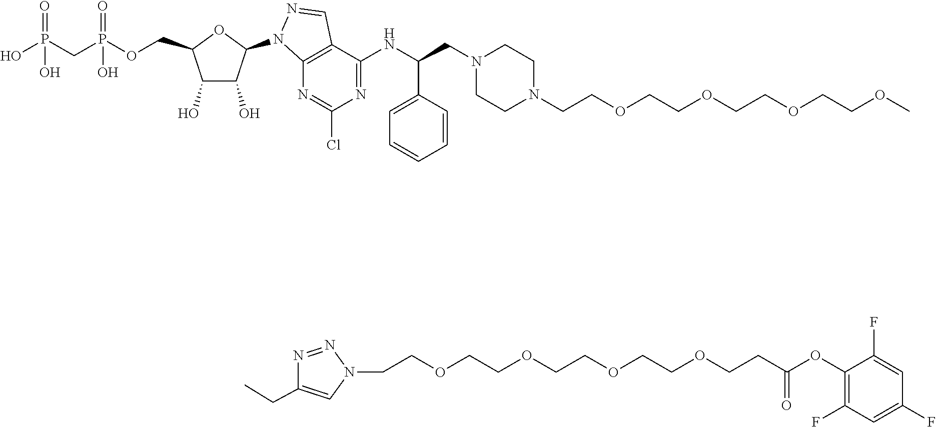 |
| Int-109 | 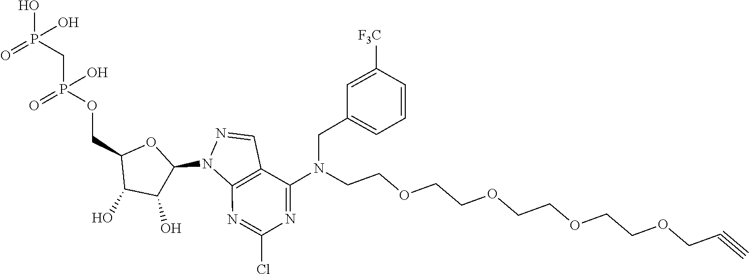 [SMILES: C#CCOCCOCCOCCOCCN(Cc1cccc(C(F)(F)F)c1)c1nc(Cl)nc2c1cnn2[C@@H]1O[C@H](COP(=O)(O)CP(=O)(O)O)[C@@H](O)[C@H]1O] |
| Int-110 | 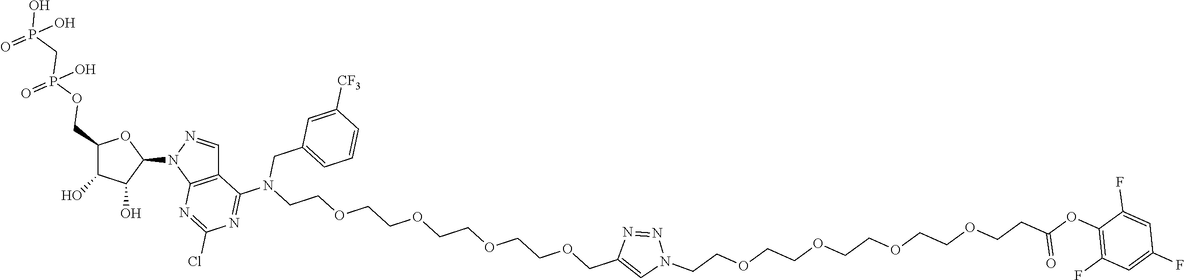 [SMILES: O=C(CCOCCOCCOCCOCCn1cc(COCCOCCOCCOCCN(Cc2cccc(C(F)(F)F)c2)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-111 | 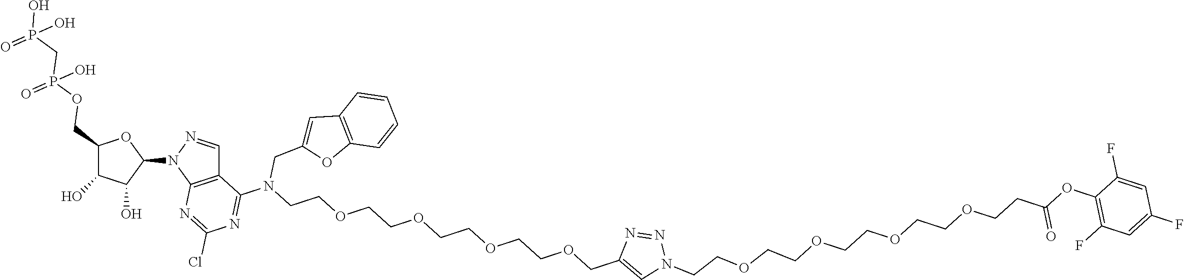 [SMILES: O=C(CCOCCOCCOCCOCCn1cc(COCCOCCOCCOCCN(Cc2cc3ccccc3o2)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-112 | 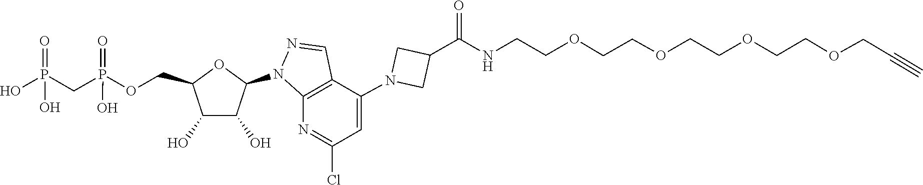 [SMILES: C#CCOCCOCCOCCOCCNC(=O)C1CN(c2cc(Cl)nc3c2cnn3[C@@H]2O[C@H](COP(=O)(O)CP(=O)(O)O)[C@@H](O)[C@H]2O)C1] |
| Int-113 | 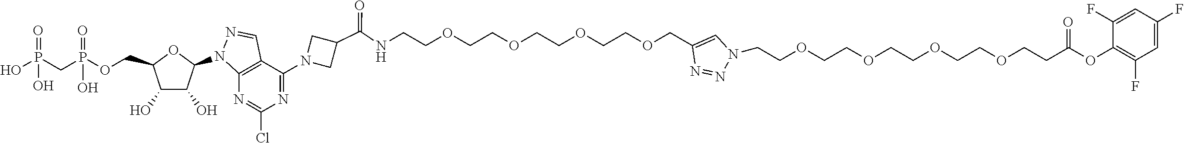 [SMILES: O=C(CCOCCOCCOCCOCCn1cc(COCCOCCOCCOCCNC(=O)C2CN(c3nc(Cl)nc4c3cnn4[C@@H]3O[C@H](COP(=O)(O)CP(=O)(O)O)[C@@H](O)[C@H]3O)C2)nn1)Oc1c(F)cc(F)cc1F] |
| Int-114 | 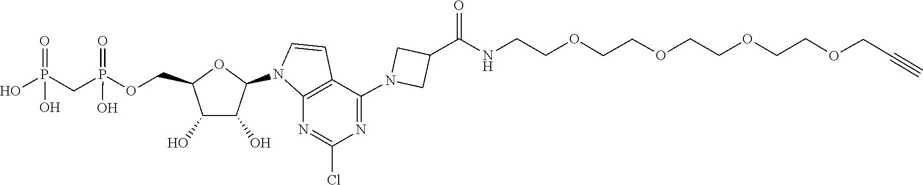 [SMILES: C#CCOCCOCCOCCOCCNC(=O)C1CN(c2nc(Cl)nc3c2ccn3[C@@H]2O[C@H](COP(=O)(O)CP(=O)(O)O)[C@@H](O)[C@H]2O)C1] |
| Int-115 | 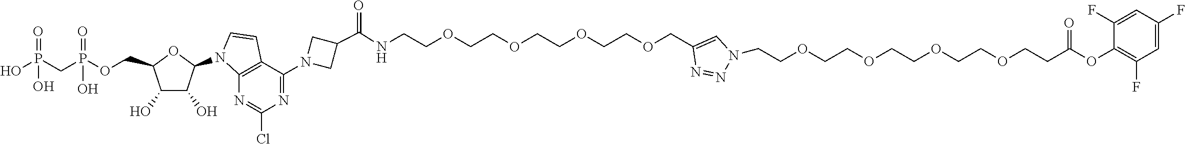 [SMILES: O=C(CCOCCOCCOCCOCCn1cc(COCCOCCOCCOCCNC(=O)C2CN(c3nc(Cl)nc4c3ccn4[C@@H]3O[C@H](COP(=O)(O)CP(=O)(O)O)[C@@H](O)[C@H]3O)C2)nn1)Oc1c(F)cc(F)cc1F] |
| Int-116 | 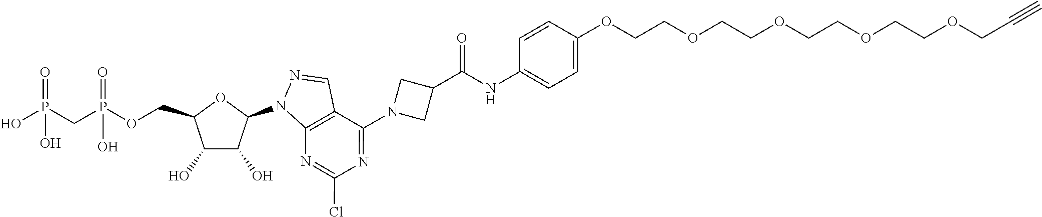 [SMILES: C#CCOCCOCCOCCOCCOc1ccc(NC(=O)C2CN(c3nc(Cl)nc4c3cnn4[C@@H]3O[C@H](COP(=O)(O)CP(=O)(O)O)[C@@H](O)[C@H]3O)C2)cc1] |
| Int-117 | 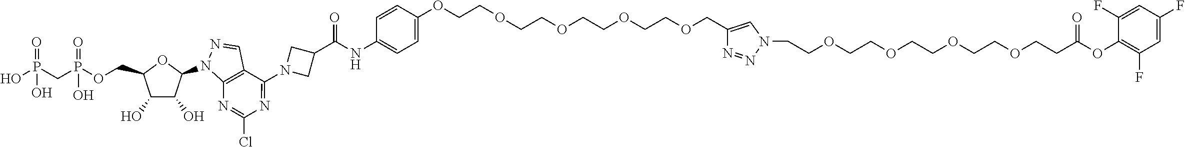 [SMILES: O=C(CCOCCOCCOCCOCCn1cc(COCCOCCOCCOCCOc2ccc(NC(=O)C3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-118 | 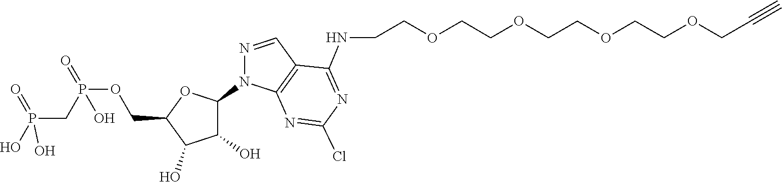 [SMILES: C#CCOCCOCCOCCOCCNc1nc(Cl)nc2c1cnn2[C@@H]1O[C@H](COP(=O)(O)CP(=O)(O)O)[C@@H](O)[C@H]1O] |
| Int-119 | 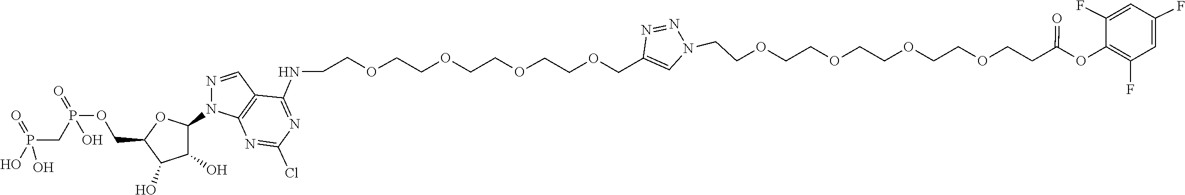 [SMILES: O=C(CCOCCOCCOCCOCCn1cc(COCCOCCOCCOCCNc2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-120 | 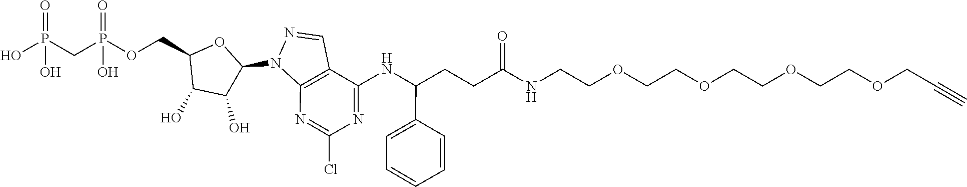 [SMILES: C#CCOCCOCCOCCOCCNC(=O)CCC(Nc1nc(Cl)nc2c1cnn2[C@@H]1O[C@H](COP(=O)(O)CP(=O)(O)O)[C@@H](O)[C@H]1O)c1ccccc1] |
| Int-121 | 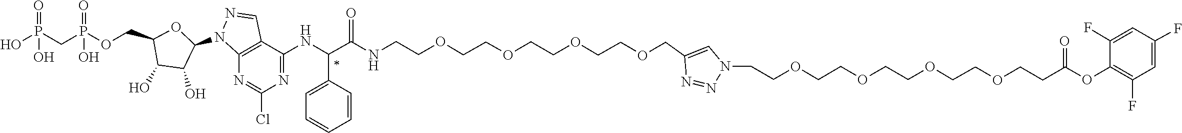 [SMILES: O=C(CCOCCOCCOCCOCCn1cc(COCCOCCOCCOCCNC(=O)C(Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F] |
| Int-122 | 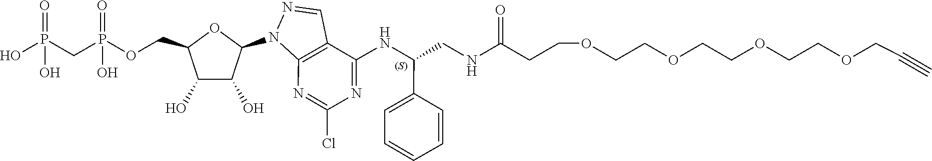 |
| Int-123 | 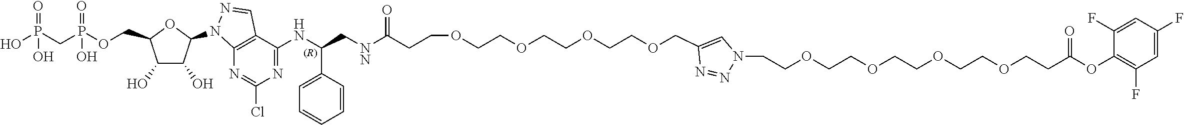 |
| Int-124 | 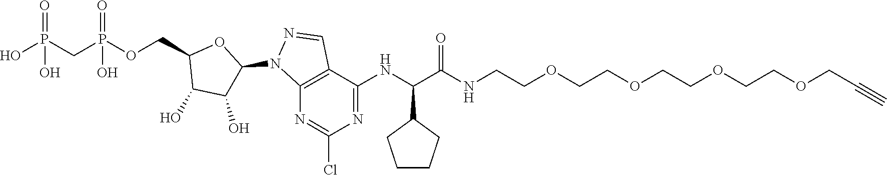 [SMILES: C#CCOCCOCCOCCOCCNC(=O)[C@H](Nc1nc(Cl)nc2c1cnn2[C@@H]1O[C@H](COP(=O)(O)CP(=O)(O)O)[C@@H](O)[C@H]1O)C1CCCC1] |
| Int-125 | 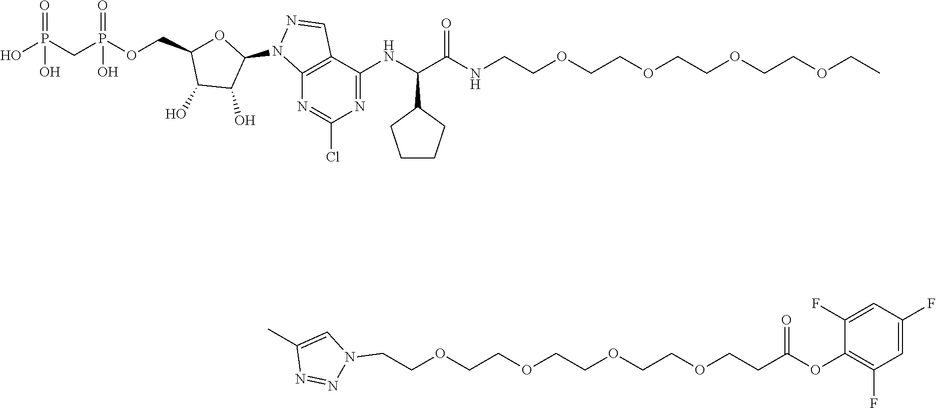 |
| Int-126 | 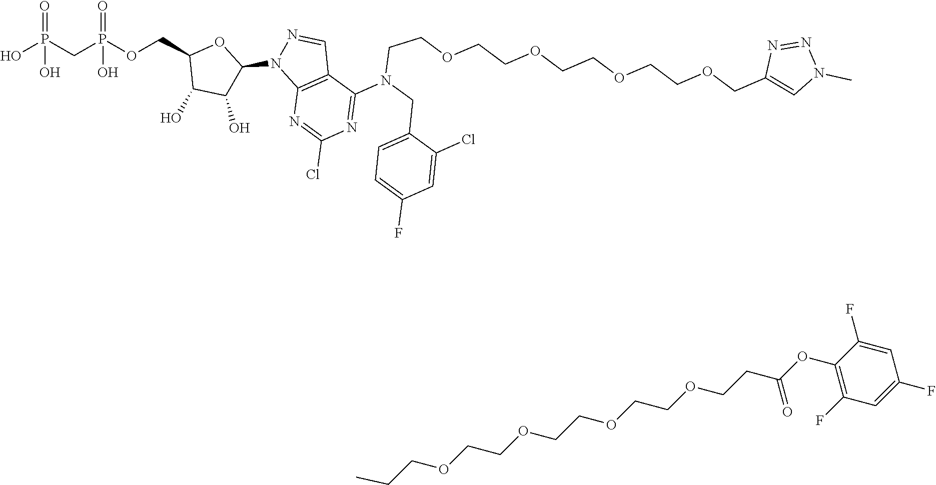 |
| Int-127 | 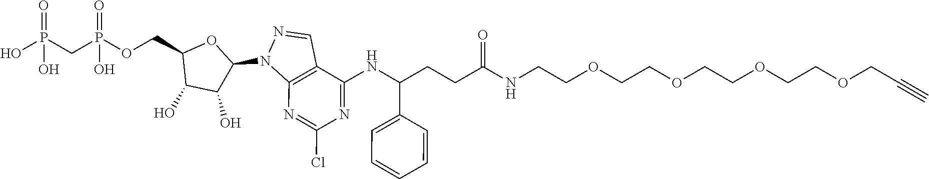 [SMILES: C#CCOCCOCCOCCOCCNC(=O)CCC(Nc1nc(Cl)nc2c1cnn2[C@@H]1O[C@H](COP(=O)(O)CP(=O)(O)O)[C@@H](O)[C@H]1O)c1ccccc1] |
| Int-128 | 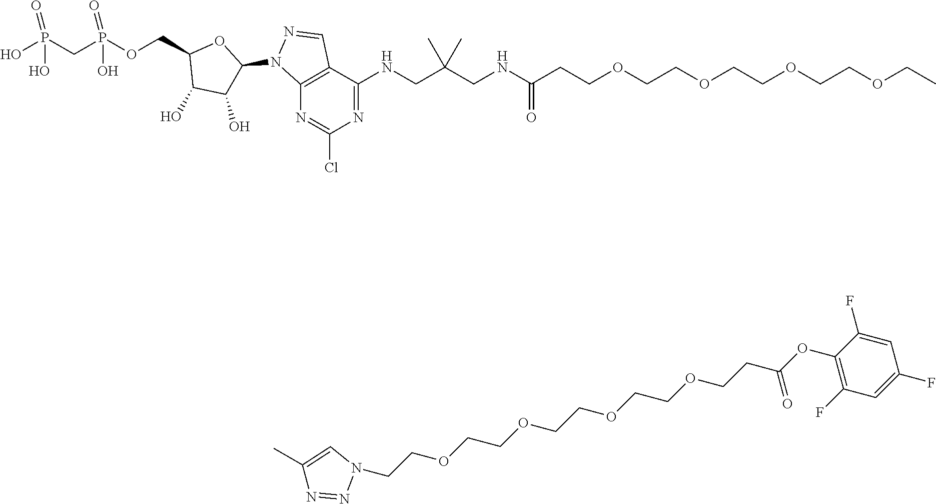 |
| Int-129 | 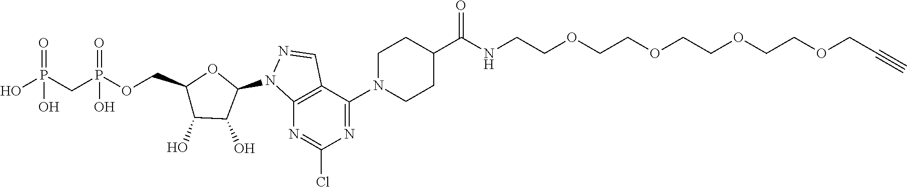 [SMILES: C#CCOCCOCCOCCOCCNC(=O)C1CCN(c2nc(Cl)nc3c2cnn3[C@@H]2O[C@H](COP(=O)(O)CP(=O)(O)O)[C@@H](O)[C@H]2O)CC1] |
| Int-130 | 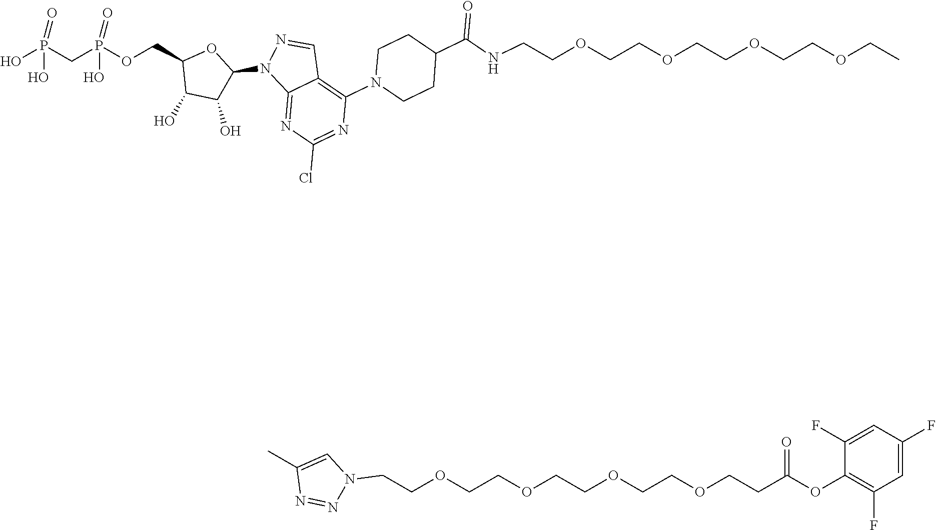 |
| Int-131 | 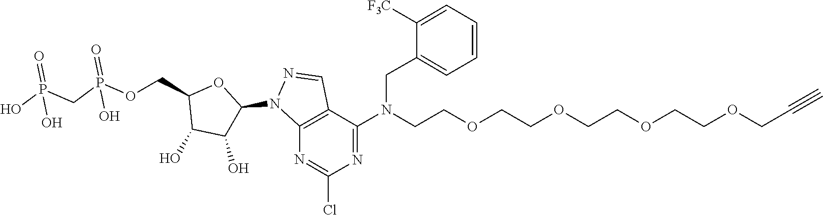 [SMILES: C#CCOCCOCCOCCOCCN(Cc1ccccc1C(F)(F)F)c1nc(Cl)nc2c1cnn2[C@@H]1O[C@H](COP(=O)(O)CP(=O)(O)O)[C@@H](O)[C@H]1O] |
| Int-132 | 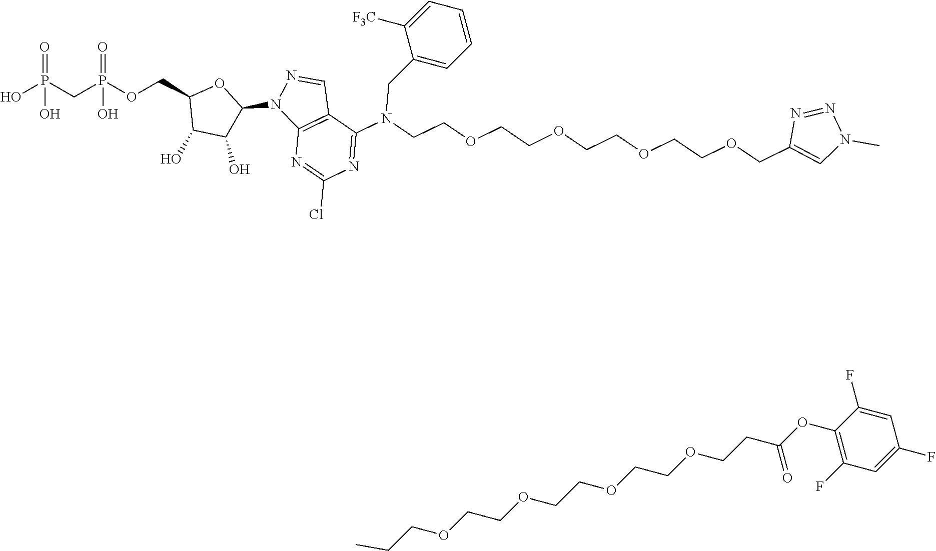 |
| Int-133 | 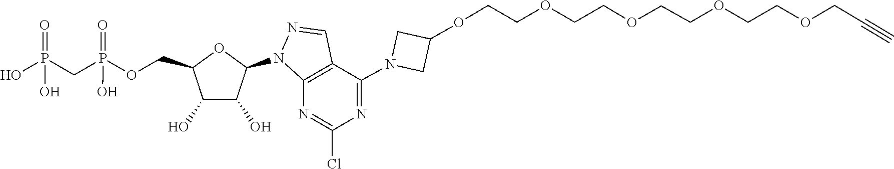 [SMILES: C#CCOCCOCCOCCOCCOC1CN(c2nc(Cl)nc3c2cnn3[C@@H]2O[C@H](COP(=O)(O)CP(=O)(O)O)[C@@H](O)[C@H]2O)C1] |
| Int-134 | 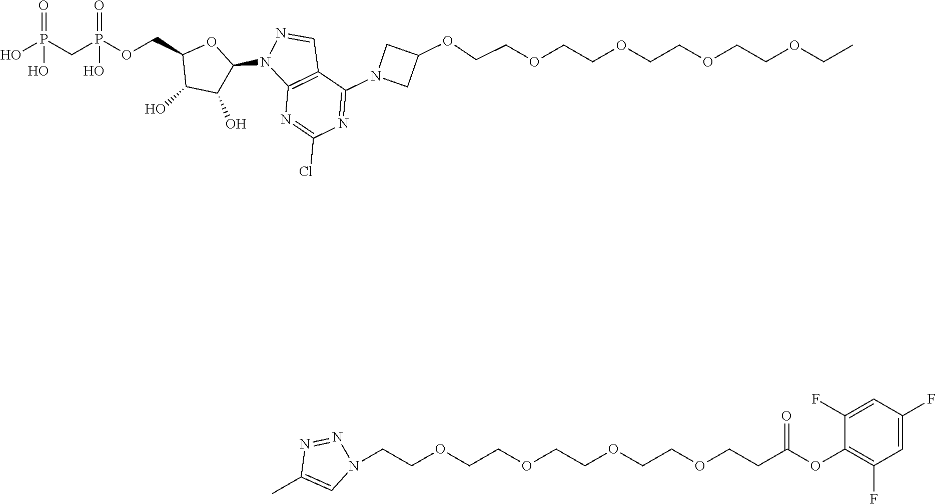 |
| Int-135 | 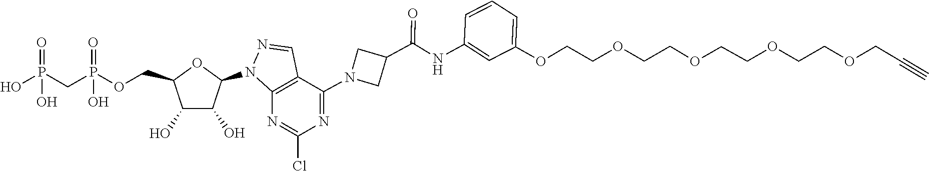 [SMILES: C#CCOCCOCCOCCOCCOc1cccc(NC(=O)C2CN(c3nc(Cl)nc4c3cnn4[C@@H]3O[C@H](COP(=O)(O)CP(=O)(O)O)[C@@H](O)[C@H]3O)C2)c1] |
| Int-136 | 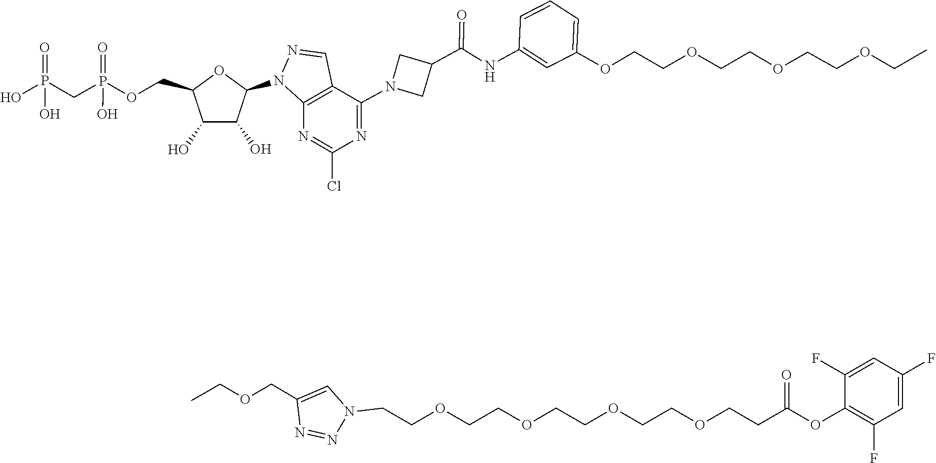 |
| Int-137 | 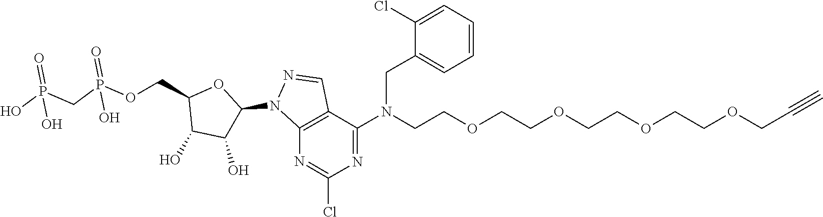 [SMILES: C#CCOCCOCCOCCOCCN(Cc1ccccc1Cl)c1nc(Cl)nc2c1cnn2[C@@H]1O[C@H](COP(=O)(O)CP(=O)(O)O)[C@@H](O)[C@H]1O] |
| Int-138 | 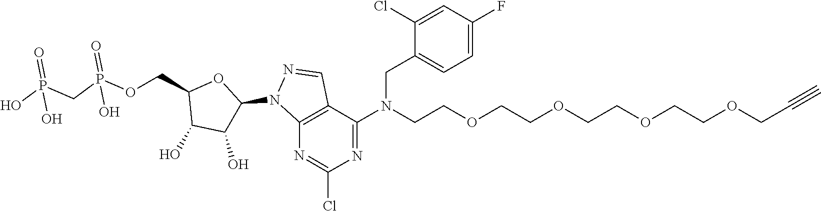 [SMILES: C#CCOCCOCCOCCOCCN(Cc1ccc(F)cc1Cl)c1nc(Cl)nc2c1cnn2[C@@H]1O[C@H](COP(=O)(O)CP(=O)(O)O)[C@@H](O)[C@H]1O] |
| Int-139 | 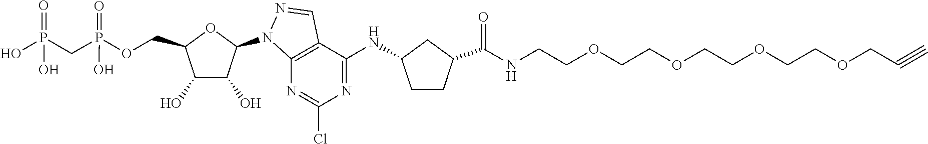 [SMILES: C#CCOCCOCCOCCOCCNC(=O)[C@@H]1CC[C@H](Nc2nc(Cl)nc3c2cnn3[C@@H]2O[C@H](COP(=O)(O)CP(=O)(O)O)[C@@H](O)[C@H]2O)C1] |
| Int-140 | 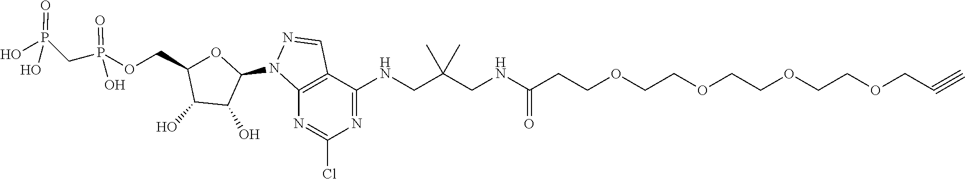 [SMILES: C#CCOCCOCCOCCOCCC(=O)NCC(C)(C)CNc1nc(Cl)nc2c1cnn2[C@@H]1O[C@H](COP(=O)(O)CP(=O)(O)O)[C@@H](O)[C@H]1O] |
| Int-141 | 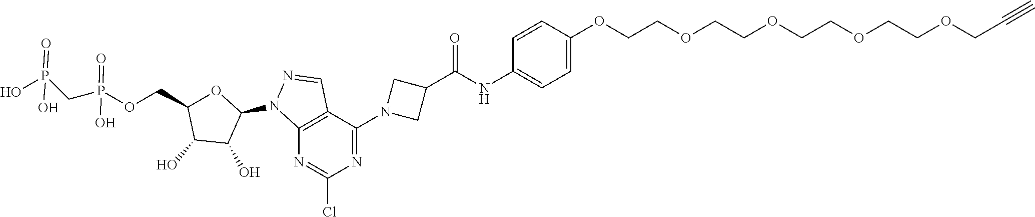 [SMILES: C#CCOCCOCCOCCOCCOc1ccc(NC(=O)C2CN(c3nc(Cl)nc4c3cnn4[C@@H]3O[C@H](COP(=O)(O)CP(=O)(O)O)[C@@H](O)[C@H]3O)C2)cc1] |
| Int-142 | 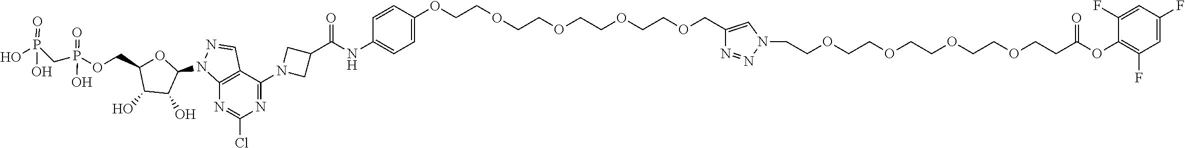 [SMILES: O=C(CCOCCOCCOCCOCCn1cc(COCCOCCOCCOCCOc2ccc(NC(=O)C3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-143 | 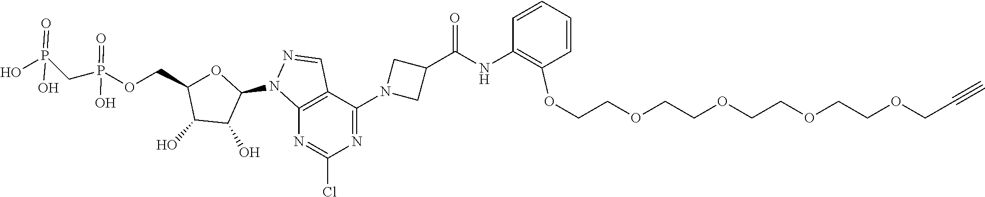 [SMILES: C#CCOCCOCCOCCOCCOc1ccccc1NC(=O)C1CN(c2nc(Cl)nc3c2cnn3[C@@H]2O[C@H](COP(=O)(O)CP(=O)(O)O)[C@@H](O)[C@H]2O)C1] |
| Int-144 | 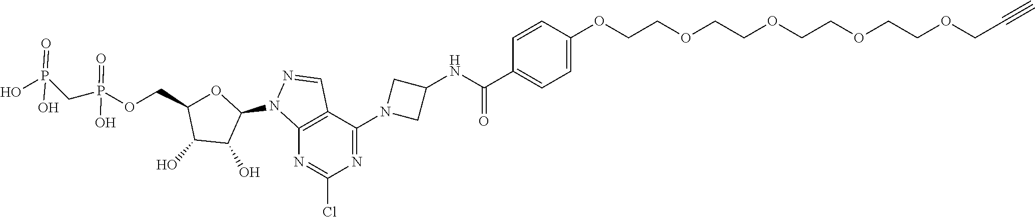 [SMILES: C#CCOCCOCCOCCOCCOc1ccc(C(=O)NC2CN(c3nc(Cl)nc4c3cnn4[C@@H]3O[C@H](COP(=O)(O)CP(=O)(O)O)[C@@H](O)[C@H]3O)C2)cc1] |
| Int-145 | 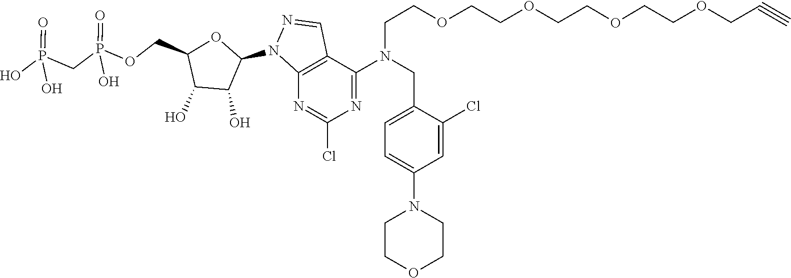 [SMILES: C#CCOCCOCCOCCOCCN(Cc1ccc(N2CCOCC2)cc1Cl)c1nc(Cl)nc2c1cnn2[C@@H]1O[C@H](COP(=O)(O)CP(=O)(O)O)[C@@H](O)[C@H]1O] |
| Int-146 | 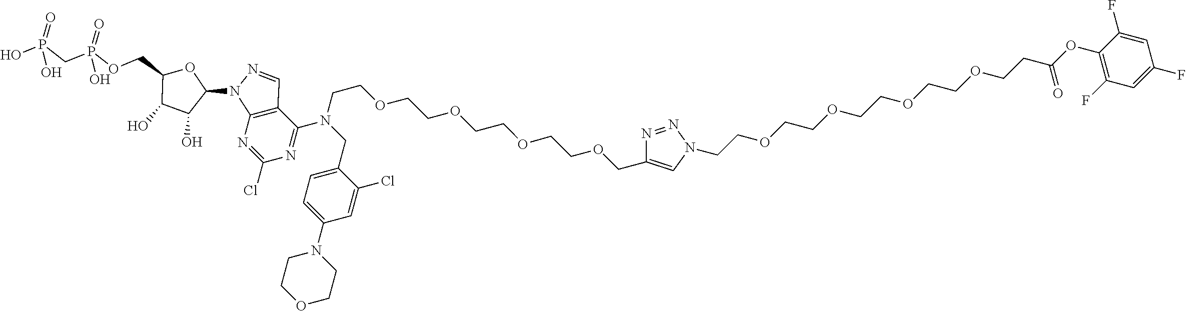 [SMILES: O=C(CCOCCOCCOCCOCCn1cc(COCCOCCOCCOCCN(Cc2ccc(N3CCOCC3)cc2Cl)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-147 | 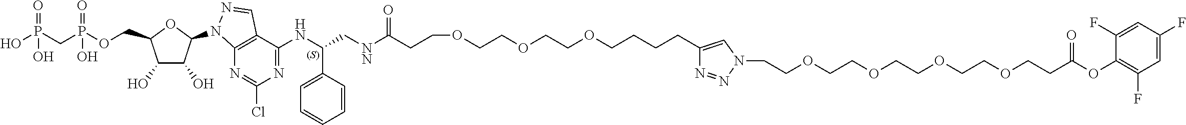 |
| Int-148 | 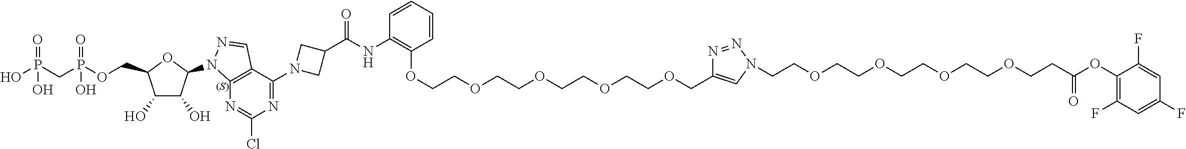 |
| Int-149 | 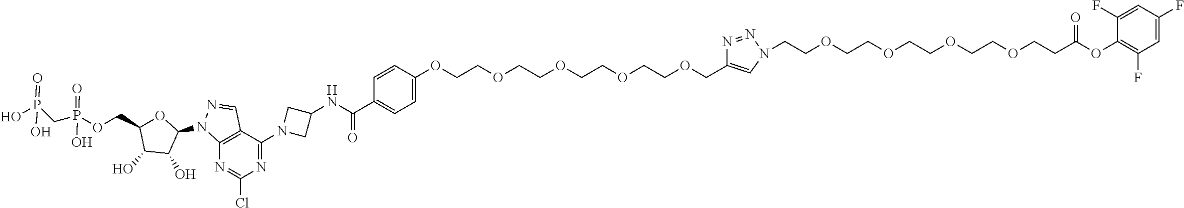 [SMILES: O=C(CCOCCOCCOCCOCCn1cc(COCCOCCOCCOCCOc2ccc(C(=O)NC3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-150 | 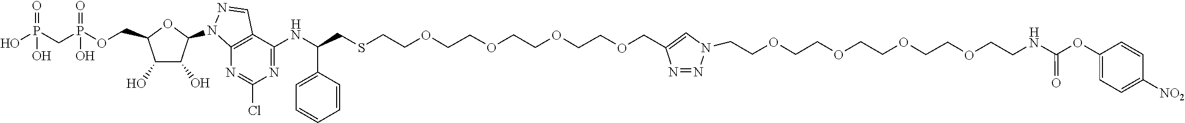 [SMILES: O=C(NCCOCCOCCOCCOCCn1cc(COCCOCCOCCOCCSC[C@H](Nc2nc(Cl)nc3c2cnn3[C@@H]2O[C@H](COP(=O)(O)CP(=O)(O)O)[C@@H](O)[C@H]2O)c2ccccc2)nn1)Oc1ccc([N+](=O)[O-])cc1] |
| Int-151 | 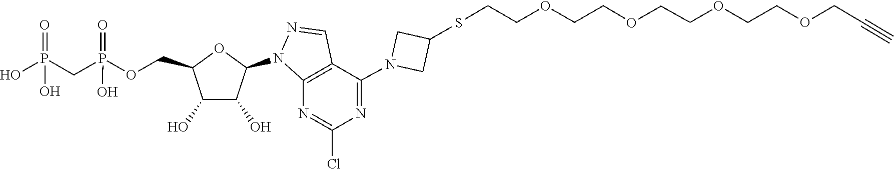 [SMILES: C#CCOCCOCCOCCOCCSC1CN(c2nc(Cl)nc3c2cnn3[C@@H]2O[C@H](COP(=O)(O)CP(=O)(O)O)[C@@H](O)[C@H]2O)C1] |
| Int-152 | 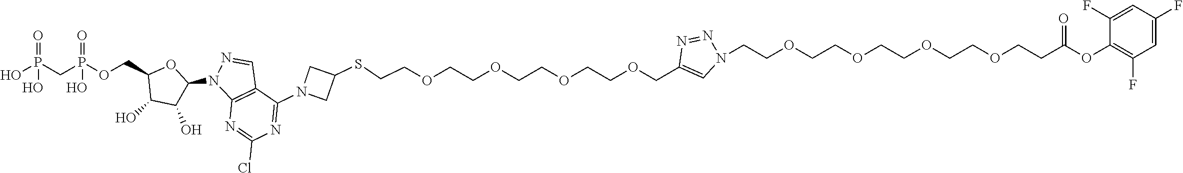 [SMILES: O=C(CCOCCOCCOCCOCCn1cc(COCCOCCOCCOCCSC2CN(c3nc(Cl)nc4c3cnn4[C@@H]3O[C@H](COP(=O)(O)CP(=O)(O)O)[C@@H](O)[C@H]3O)C2)nn1)Oc1c(F)cc(F)cc1F] |
| Int-153 | 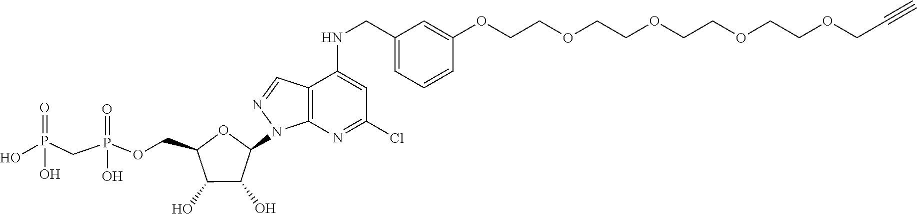 |
| Int-154 | 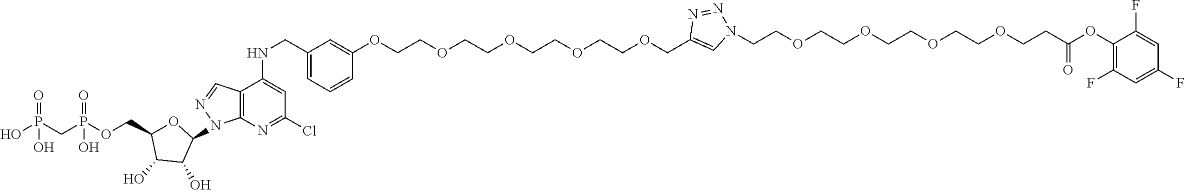 [SMILES: O=C(CCOCCOCCOCCOCCn1cc(COCCOCCOCCOCCOc2cccc(CNc3cc(Cl)nc4c3cnn4[C@@H]3O[C@H](COP(=O)(O)CP(=O)(O)O)[C@@H](O)[C@H]3O)c2)nn1)Oc1c(F)cc(F)cc1F] |
| Int-155 | 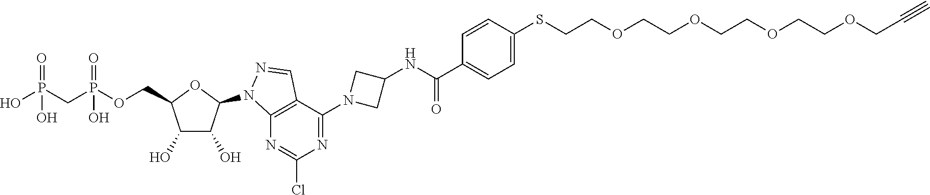 [SMILES: C#CCOCCOCCOCCOCCSc1ccc(C(=O)NC2CN(c3nc(Cl)nc4c3cnn4[C@@H]3O[C@H](COP(=O)(O)CP(=O)(O)O)[C@@H](O)[C@H]3O)C2)cc1] |
| Int-156 | 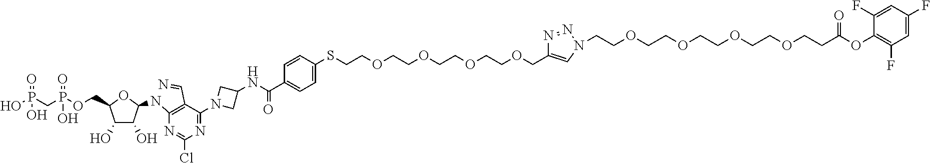 [SMILES: O=C(CCOCCOCCOCCOCCn1cc(COCCOCCOCCOCCSc2ccc(C(=O)NC3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-157 | 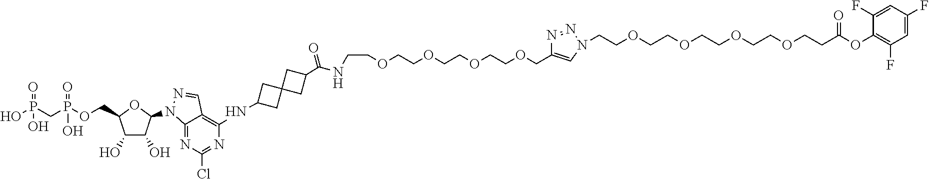 [SMILES: O=C(CCOCCOCCOCCOCCn1cc(COCCOCCOCCOCCNC(=O)C2CC3(CC(Nc4nc(Cl)nc5c4cnn5[C@@H]4O[C@H](COP(=O)(O)CP(=O)(O)O)[C@@H](O)[C@H]4O)C3)C2)nn1)Oc1c(F)cc(F)cc1F] |
| Int-158 | 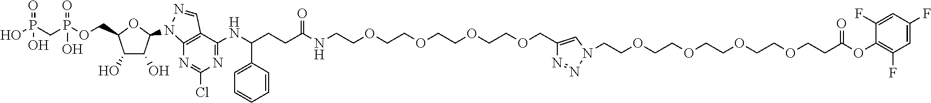 [SMILES: O=C(CCC(Nc1nc(Cl)nc2c1cnn2[C@@H]1O[C@H](COP(=O)(O)CP(=O)(O)O)[C@@H](O)[C@H]1O)c1ccccc1)NCCOCCOCCOCCOCc1cn(CCOCCOCCOCCOCCC(=O)Oc2c(F)cc(F)cc2F)nn1] |
| Int-159 | 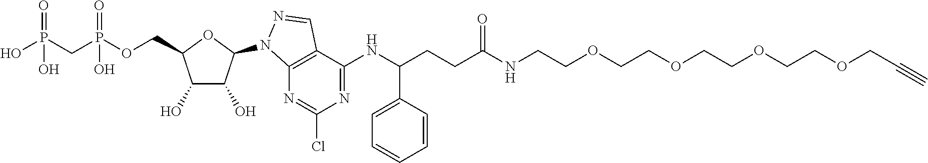 [SMILES: C#CCOCCOCCOCCOCCNC(=O)CCC(Nc1nc(Cl)nc2c1cnn2[C@@H]1O[C@H](COP(=O)(O)CP(=O)(O)O)[C@@H](O)[C@H]1O)c1ccccc1] |
| Int-160 | 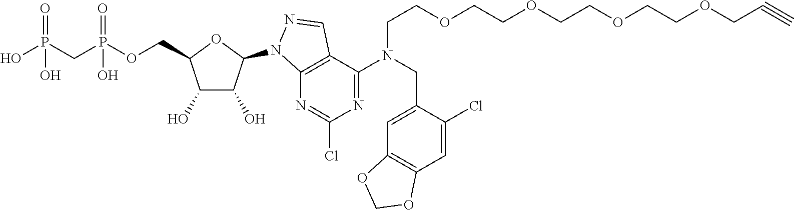 [SMILES: C#CCOCCOCCOCCOCCN(Cc1cc2c(cc1Cl)OCO2)c1nc(Cl)nc2c1cnn2[C@@H]1O[C@H](COP(=O)(O)CP(=O)(O)O)[C@@H](O)[C@H]1O] |
| Int-161 | 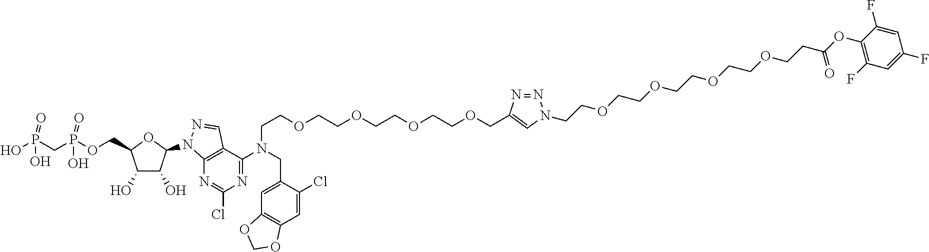 [SMILES: O=C(CCOCCOCCOCCOCCn1cc(COCCOCCOCCOCCN(Cc2cc3c(cc2Cl)OCO3)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-162 | 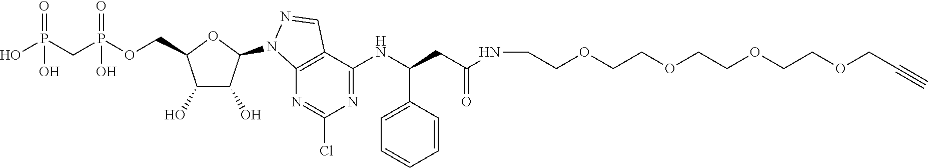 [SMILES: C#CCOCCOCCOCCOCCNC(=O)C[C@H](Nc1nc(Cl)nc2c1cnn2[C@@H]1O[C@H](COP(=O)(O)CP(=O)(O)O)[C@@H](O)[C@H]1O)c1ccccc1] |
| Int-163 | 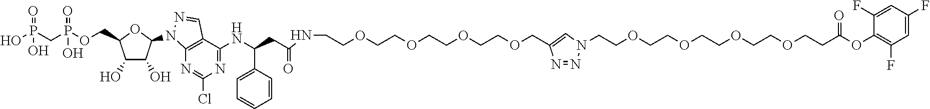 [SMILES: O=C(C[C@H](Nc1nc(Cl)nc2c1cnn2[C@@H]1O[C@H](COP(=O)(O)CP(=O)(O)O)[C@@H](O)[C@H]1O)c1ccccc1)NCCOCCOCCOCCOCc1cn(CCOCCOCCOCCOCCC(=O)Oc2c(F)cc(F)cc2F)nn1] |
| Int-164 | 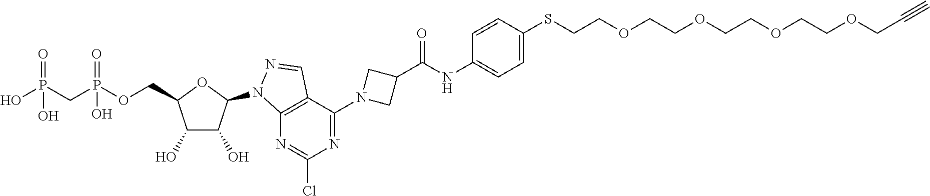 [SMILES: C#CCOCCOCCOCCOCCSc1ccc(NC(=O)C2CN(c3nc(Cl)nc4c3cnn4[C@@H]3O[C@H](COP(=O)(O)CP(=O)(O)O)[C@@H](O)[C@H]3O)C2)cc1] |
| Int-165 | 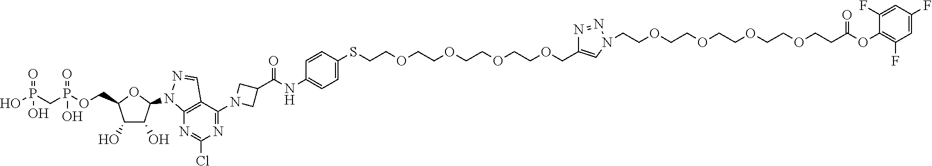 [SMILES: O=C(CCOCCOCCOCCOCCn1cc(COCCOCCOCCOCCSc2ccc(NC(=O)C3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-166 | 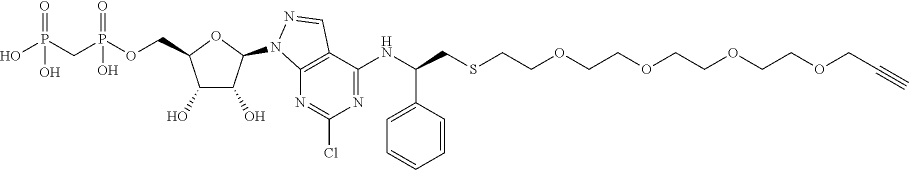 [SMILES: C#CCOCCOCCOCCOCCSC[C@H](Nc1nc(Cl)nc2c1cnn2[C@@H]1O[C@H](COP(=O)(O)CP(=O)(O)O)[C@@H](O)[C@H]1O)c1ccccc1] |
| Int-167 | 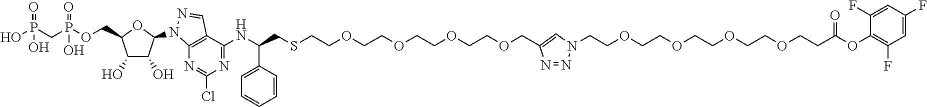 [SMILES: O=C(CCOCCOCCOCCOCCn1cc(COCCOCCOCCOCCSC[C@H](Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F] |
| Int-168 | 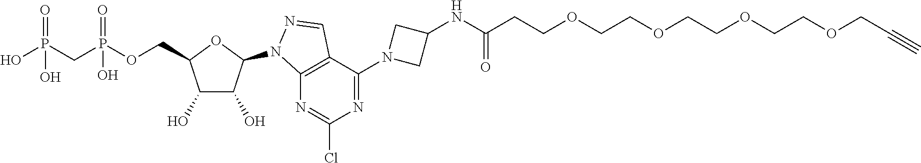 [SMILES: C#CCOCCOCCOCCOCCC(=O)NC1CN(c2nc(Cl)nc3c2cnn3[C@@H]2O[C@H](COP(=O)(O)CP(=O)(O)O)[C@@H](O)[C@H]2O)C1] |
| Int-169 | 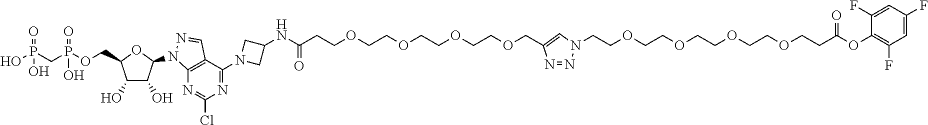 [SMILES: O=C(CCOCCOCCOCCOCc1cn(CCOCCOCCOCCOCCC(=O)Oc2c(F)cc(F)cc2F)nn1)NC1CN(c2nc(Cl)nc3c2cnn3[C@@H]2O[C@H](COP(=O)(O)CP(=O)(O)O)[C@@H](O)[C@H]2O)C1] |
| Int-170 | 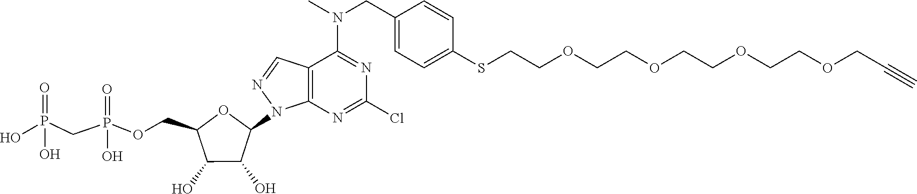 |
| Int-171 | 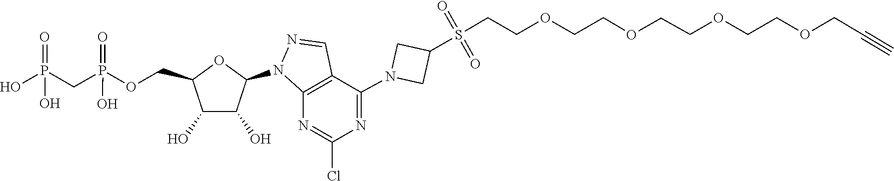 [SMILES: C#CCOCCOCCOCCOCCS(=O)(=O)C1CN(c2nc(Cl)nc3c2cnn3[C@@H]2O[C@H](COP(=O)(O)CP(=O)(O)O)[C@@H](O)[C@H]2O)C1] |
| Int-172 | 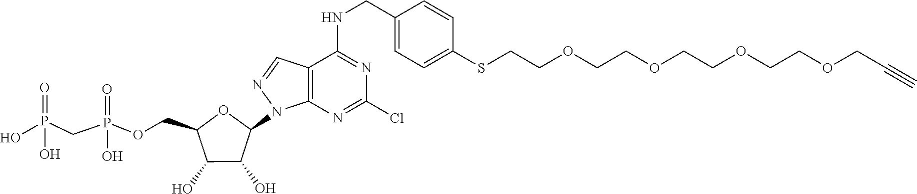 |
| Int-173 | 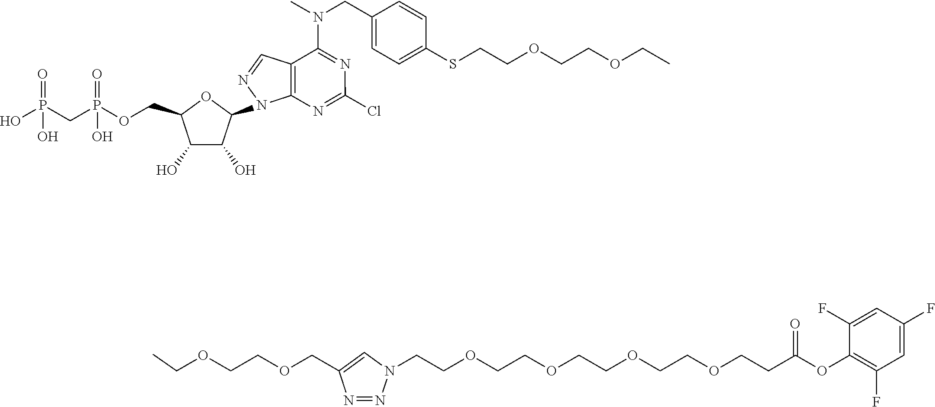 |
| Int-174 | 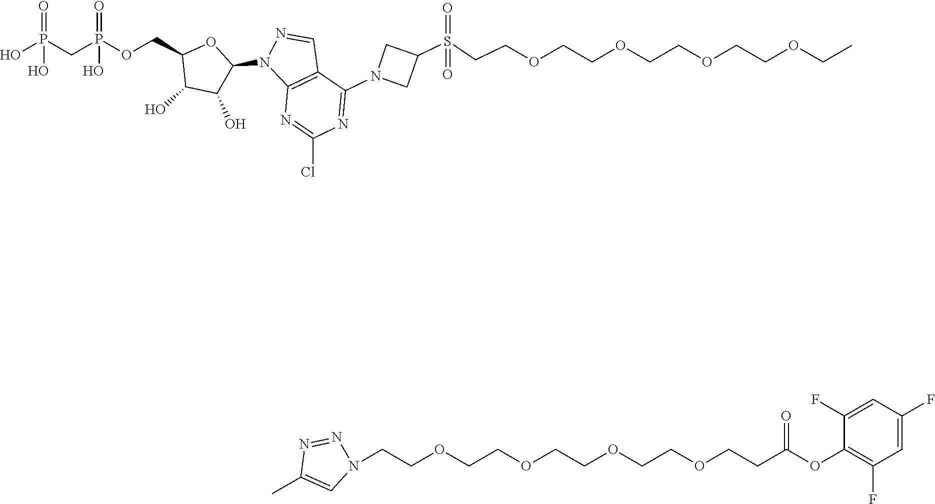 |
| Int-175 | 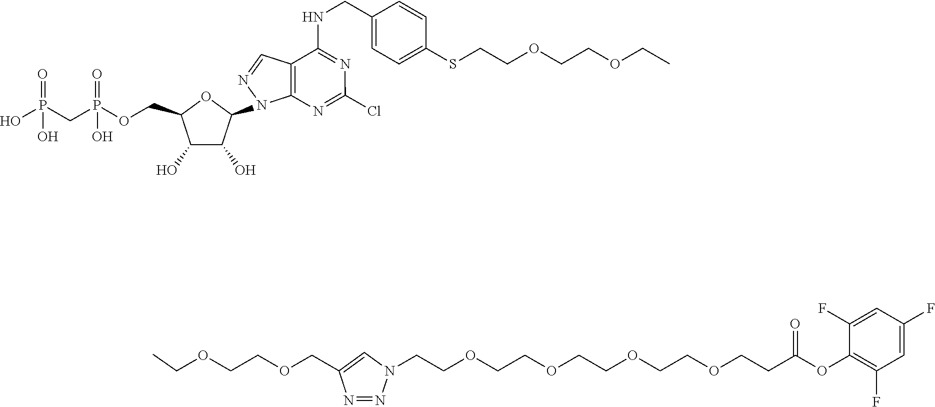 |
| Int-176 | 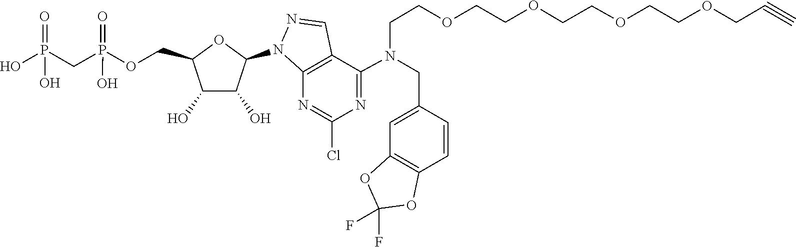 [SMILES: C#CCOCCOCCOCCOCCN(Cc1ccc2c(c1)OC(F)(F)O2)c1nc(Cl)nc2c1cnn2[C@@H]1O[C@H](COP(=O)(O)CP(=O)(O)O)[C@@H](O)[C@H]1O] |
| Int-177 | 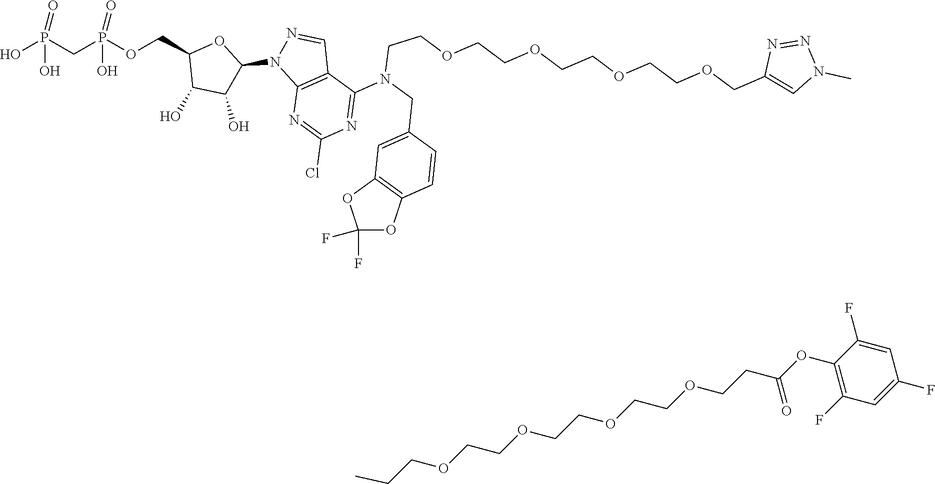 |
| Int-178 | 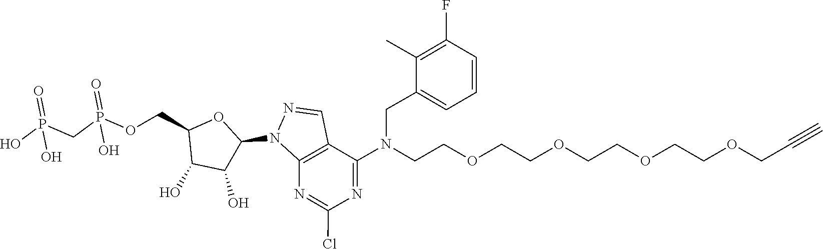 [SMILES: C#CCOCCOCCOCCOCCN(Cc1cccc(F)c1C)c1nc(Cl)nc2c1cnn2[C@@H]1O[C@H](COP(=O)(O)CP(=O)(O)O)[C@@H](O)[C@H]1O] |
| Int-179 | 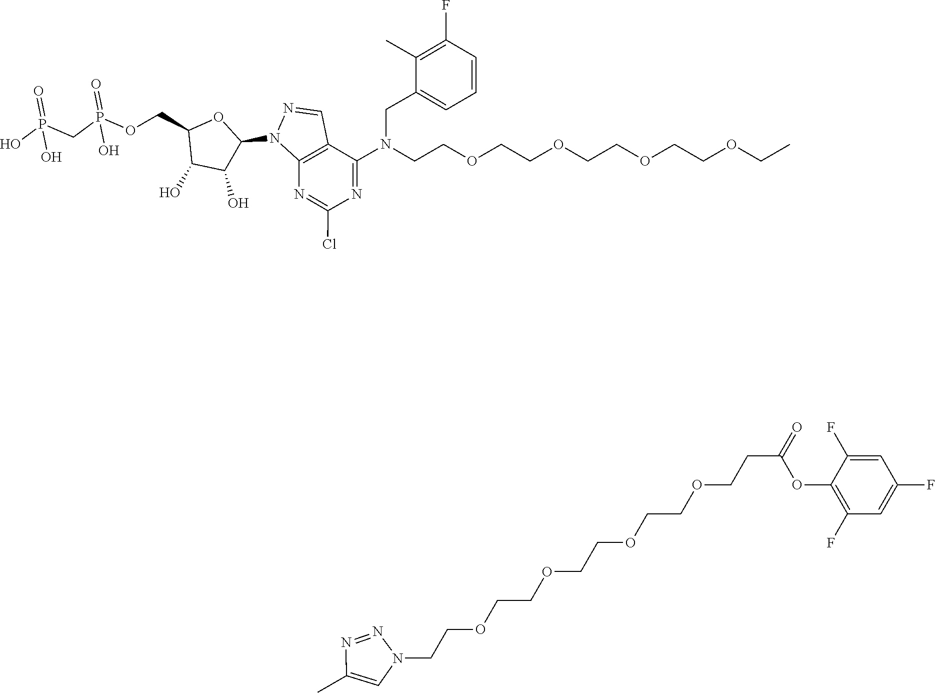 |
| Int-180 | 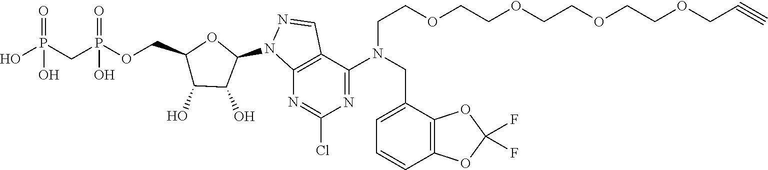 [SMILES: C#CCOCCOCCOCCOCCN(Cc1cccc2c1OC(F)(F)O2)c1nc(Cl)nc2c1cnn2[C@@H]1O[C@H](COP(=O)(O)CP(=O)(O)O)[C@@H](O)[C@H]1O] |
| Int-181 | 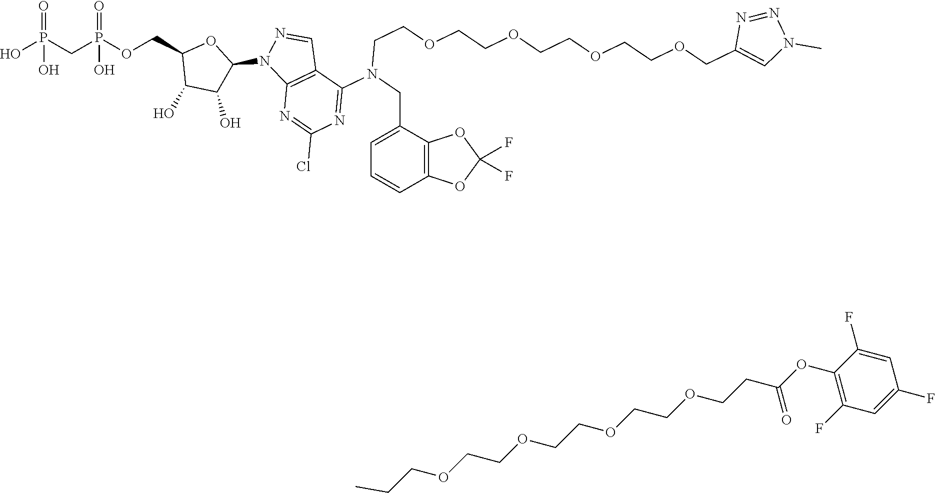 |
| Int-182 | 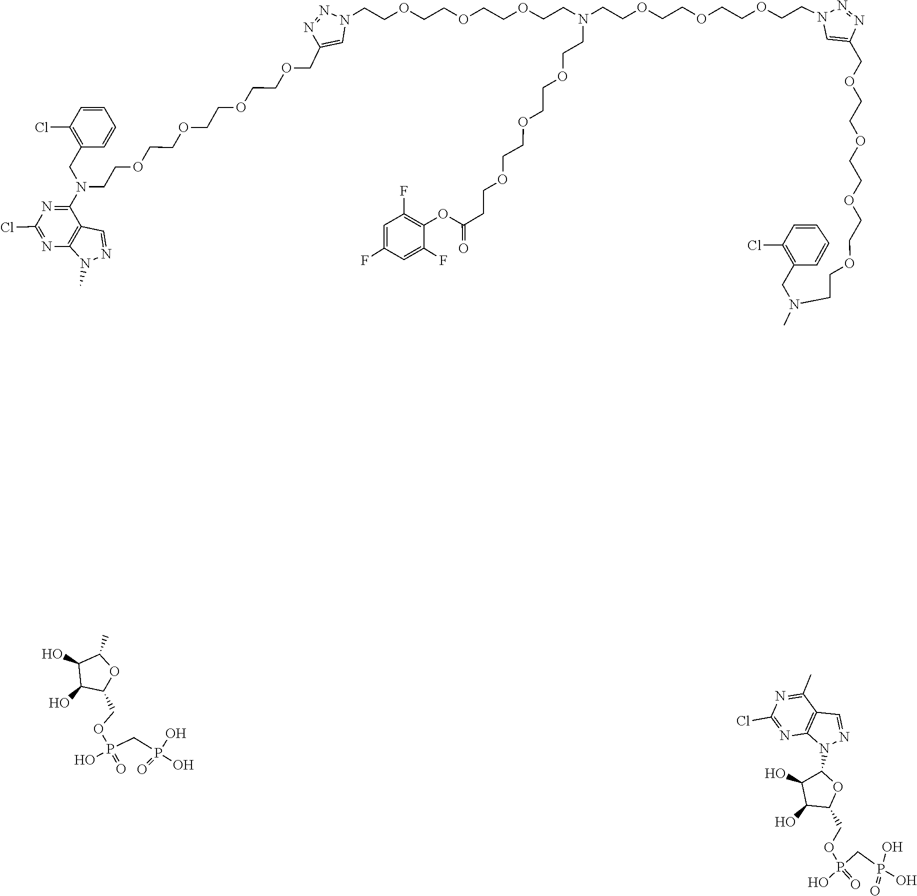 |
| Int-183 | 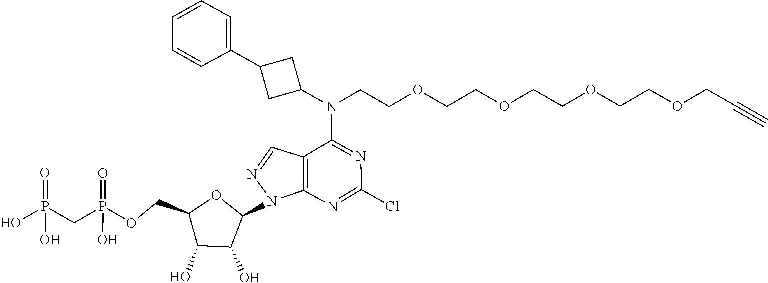 [SMILES: C#CCOCCOCCOCCOCCN(c1nc(Cl)nc2c1cnn2[C@@H]1O[C@H](COP(=O)(O)CP(=O)(O)O)[C@@H](O)[C@H]1O)C1CC(c2ccccc2)C1] |
| Int-184 | 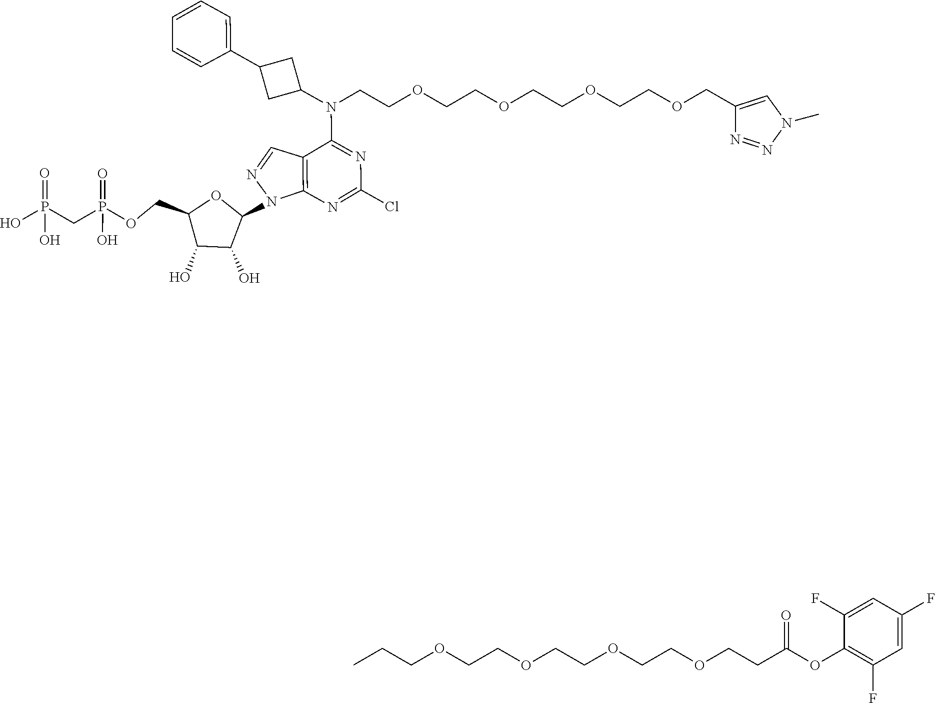 |
| Int-185 | 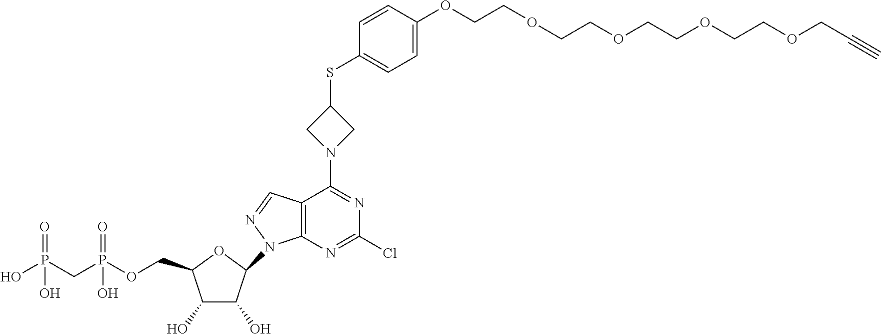 [SMILES: C#CCOCCOCCOCCOCCOc1ccc(SC2CN(c3nc(Cl)nc4c3cnn4[C@@H]3O[C@H](COP(=O)(O)CP(=O)(O)O)[C@@H](O)[C@H]3O)C2)cc1] |
| Int-186 | 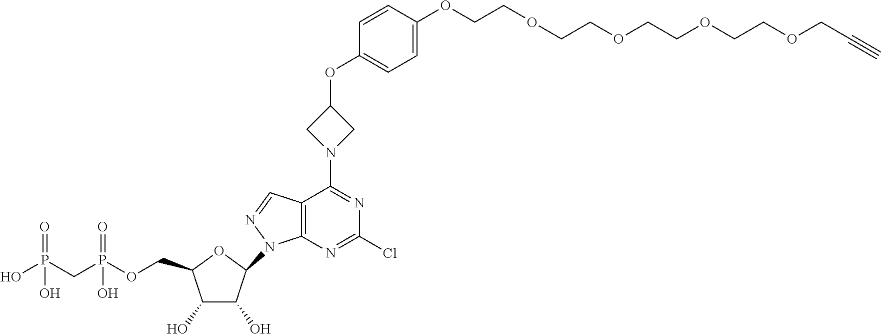 [SMILES: C#CCOCCOCCOCCOCCOc1ccc(OC2CN(c3nc(Cl)nc4c3cnn4[C@@H]3O[C@H](COP(=O)(O)CP(=O)(O)O)[C@@H](O)[C@H]3O)C2)cc1] |
| Int-187 | 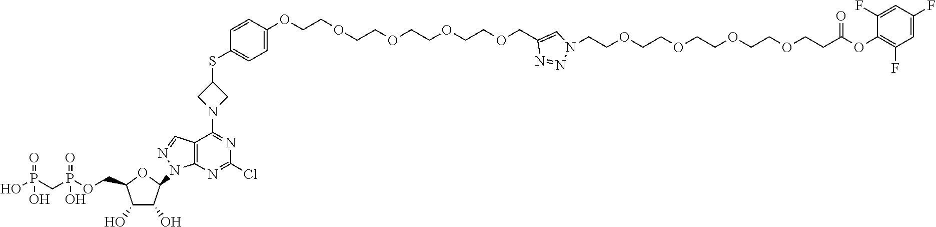 [SMILES: O=C(CCOCCOCCOCCOCCn1cc(COCCOCCOCCOCCOc2ccc(SC3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-188 | 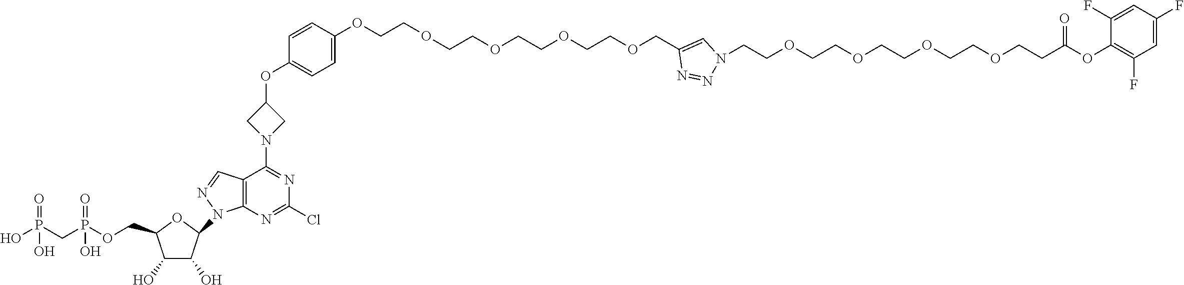 [SMILES: O=C(CCOCCOCCOCCOCCn1cc(COCCOCCOCCOCCOc2ccc(OC3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-189 | 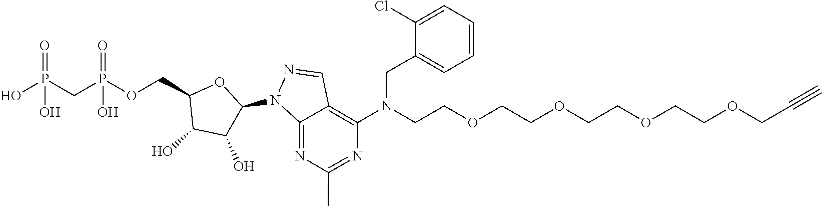 [SMILES: C#CCOCCOCCOCCOCCN(Cc1ccccc1Cl)c1nc(C)nc2c1cnn2[C@@H]1O[C@H](COP(=O)(O)CP(=O)(O)O)[C@@H](O)[C@H]1O] |
| Int-190 | 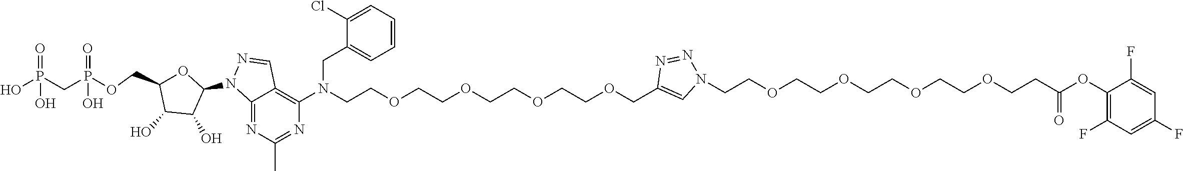 [SMILES: Cc1nc(N(CCOCCOCCOCCOCc2cn(CCOCCOCCOCCOCCC(=O)Oc3c(F)cc(F)cc3F)nn2)Cc2ccccc2Cl)c2cnn([C@@H]3O[C@H](COP(=O)(O)CP(=O)(O)O)[C@@H](O)[C@H]3O)c2n1] |
| Int-191 | 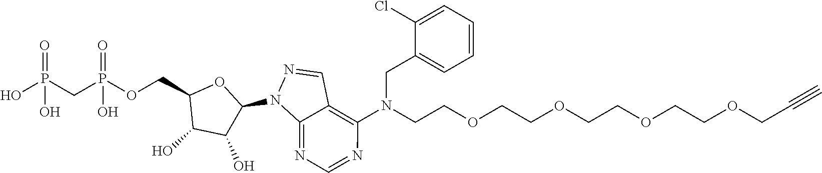 [SMILES: C#CCOCCOCCOCCOCCN(Cc1ccccc1Cl)c1ncnc2c1cnn2[C@@H]1O[C@H](COP(=O)(O)CP(=O)(O)O)[C@@H](O)[C@H]1O] |
| Int-192 | 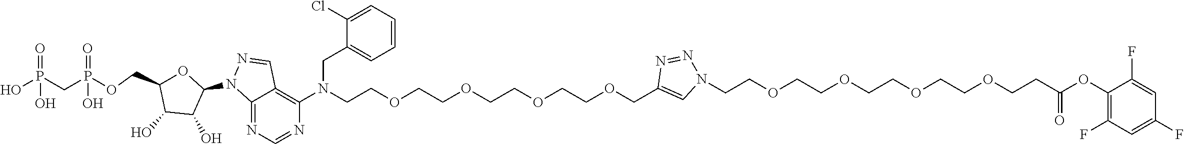 [SMILES: O=C(CCOCCOCCOCCOCCn1cc(COCCOCCOCCOCCN(Cc2ccccc2Cl)c2ncnc3c2cnn3[C@@H]2O[C@H](COP(=O)(O)CP(=O)(O)O)[C@@H](O)[C@H]2O)nn1)Oc1c(F)cc(F)cc1F] |
| Int-193 | 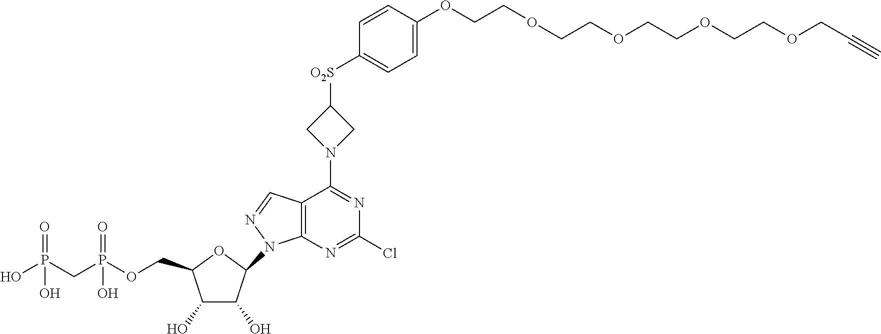 [SMILES: C#CCOCCOCCOCCOCCOc1ccc(S(=O)(=O)C2CN(c3nc(Cl)nc4c3cnn4[C@@H]3O[C@H](COP(=O)(O)CP(=O)(O)O)[C@@H](O)[C@H]3O)C2)cc1] |
| Int-194 | 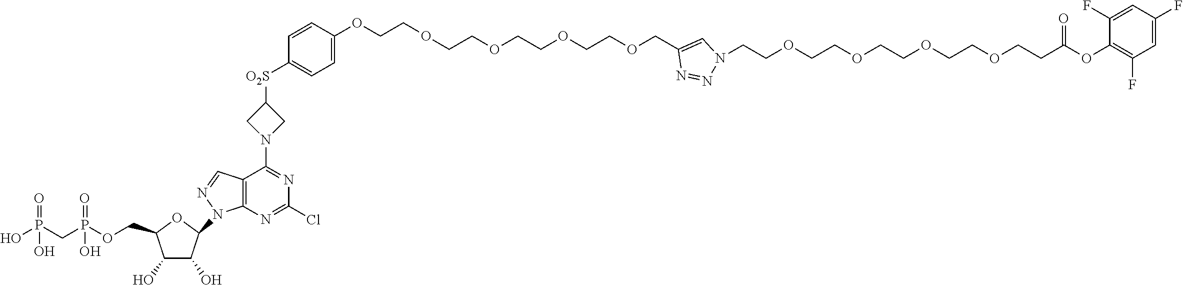 [SMILES: O=C(CCOCCOCCOCCOCCn1cc(COCCOCCOCCOCCOc2ccc(S(=O)(=O)C3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-195 | 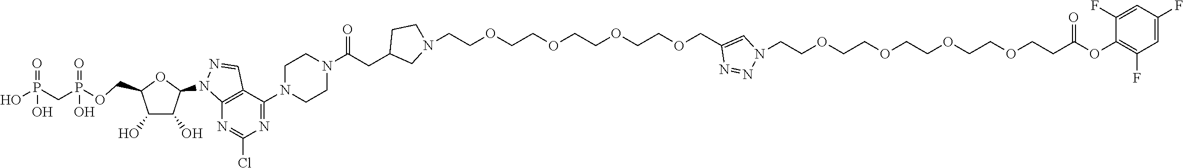 [SMILES: O=C(CCOCCOCCOCCOCCn1cc(COCCOCCOCCOCCN2CCC(CC(=O)N3CCN(c4nc(Cl)nc5c4cnn5[C@@H]4O[C@H](COP(=O)(O)CP(=O)(O)O)[C@@H](O)[C@H]4O)CC3)C2)nn1)Oc1c(F)cc(F)cc1F] |
| Int-196 | 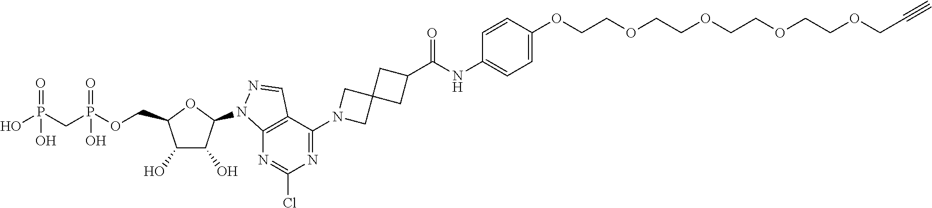 [SMILES: C#CCOCCOCCOCCOCCOc1ccc(NC(=O)C2CC3(C2)CN(c2nc(Cl)nc4c2cnn4[C@@H]2O[C@H](COP(=O)(O)CP(=O)(O)O)[C@@H](O)[C@H]2O)C3)cc1] |
| Int-197 | 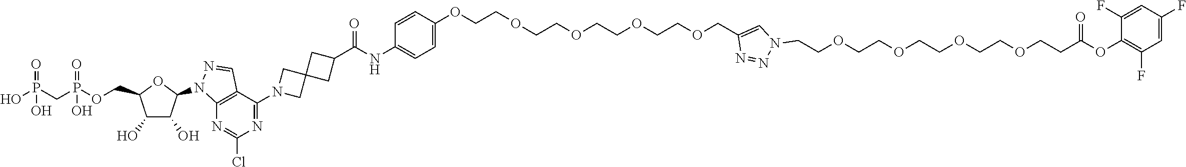 [SMILES: O=C(CCOCCOCCOCCOCCn1cc(COCCOCCOCCOCCOc2ccc(NC(=O)C3CC4(C3)CN(c3nc(Cl)nc5c3cnn5[C@@H]3O[C@H](COP(=O)(O)CP(=O)(O)O)[C@@H](O)[C@H]3O)C4)cc2)nn1)Oc1c(F)cc(F)cc1F] |
| Int-198 | 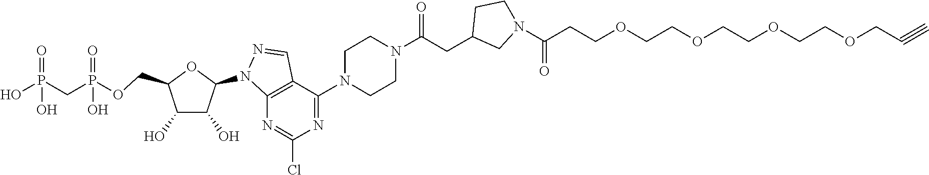 [SMILES: C#CCOCCOCCOCCOCCC(=O)N1CCC(CC(=O)N2CCN(c3nc(Cl)nc4c3cnn4[C@@H]3O[C@H](COP(=O)(O)CP(=O)(O)O)[C@@H](O)[C@H]3O)CC2)C1] |
| Int-199 | 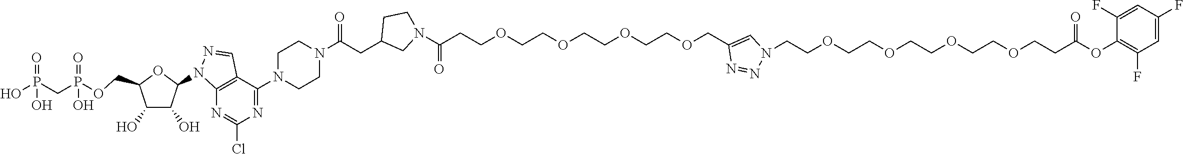 [SMILES: O=C(CCOCCOCCOCCOCCn1cc(COCCOCCOCCOCCC(=O)N2CCC(CC(=O)N3CCN(c4nc(Cl)nc5c4cnn5[C@@H]4O[C@H](COP(=O)(O)CP(=O)(O)O)[C@@H](O)[C@H]4O)CC3)C2)nn1)Oc1c(F)cc(F)cc1F] |
| Int-200 | 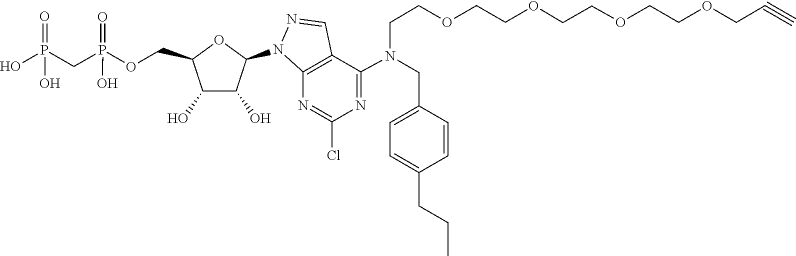 [SMILES: C#CCOCCOCCOCCOCCN(Cc1ccc(CCC)cc1)c1nc(Cl)nc2c1cnn2[C@@H]1O[C@H](COP(=O)(O)CP(=O)(O)O)[C@@H](O)[C@H]1O] |
| Int-201 | 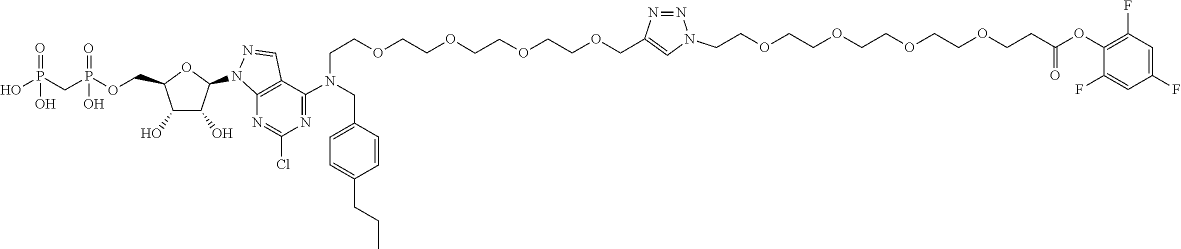 [SMILES: CCCc1ccc(CN(CCOCCOCCOCCOCc2cn(CCOCCOCCOCCOCCC(=O)Oc3c(F)cc(F)cc3F)nn2)c2nc(Cl)nc3c2cnn3[C@@H]2O[C@H](COP(=O)(O)CP(=O)(O)O)[C@@H](O)[C@H]2O)cc1] |
| Int-202 | 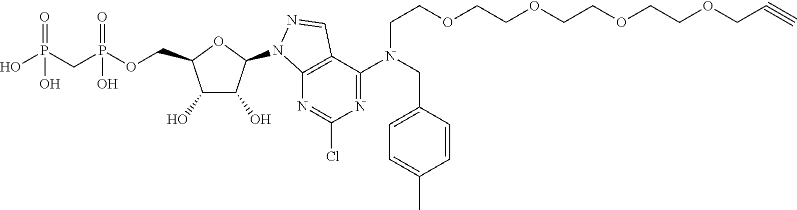 [SMILES: C#CCOCCOCCOCCOCCN(Cc1ccc(C)cc1)c1nc(Cl)nc2c1cnn2[C@@H]1O[C@H](COP(=O)(O)CP(=O)(O)O)[C@@H](O)[C@H]1O] |
| Int-203 | 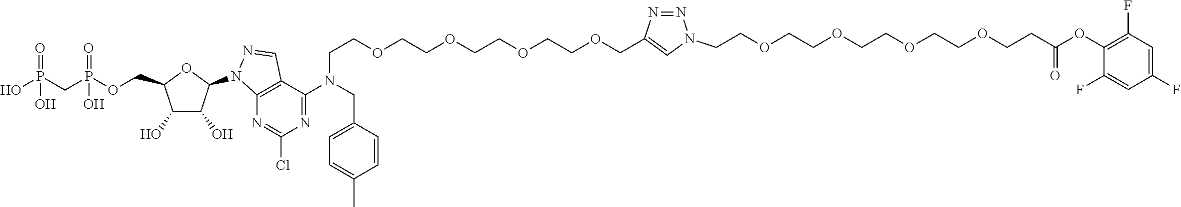 [SMILES: Cc1ccc(CN(CCOCCOCCOCCOCc2cn(CCOCCOCCOCCOCCC(=O)Oc3c(F)cc(F)cc3F)nn2)c2nc(Cl)nc3c2cnn3[C@@H]2O[C@H](COP(=O)(O)CP(=O)(O)O)[C@@H](O)[C@H]2O)cc1] |
| Int-204 | 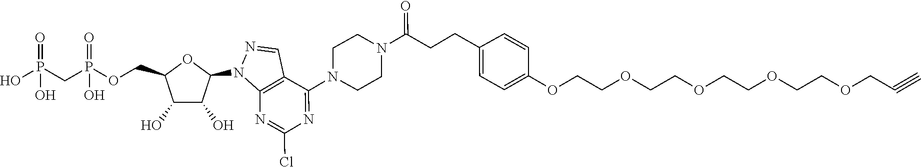 [SMILES: C#CCOCCOCCOCCOCCOc1ccc(CCC(=O)N2CCN(c3nc(Cl)nc4c3cnn4[C@@H]3O[C@H](COP(=O)(O)CP(=O)(O)O)[C@@H](O)[C@H]3O)CC2)cc1] |
| Int-205 | 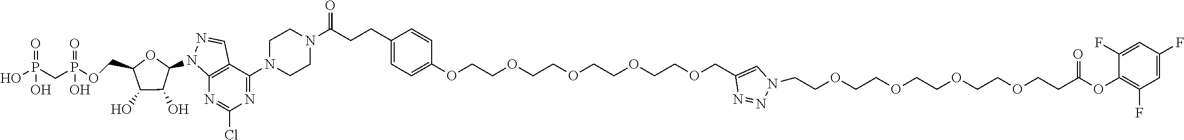 [SMILES: O=C(CCOCCOCCOCCOCCn1cc(COCCOCCOCCOCCOc2ccc(CCC(=O)N3CCN(c4nc(Cl)nc5c4cnn5[C@@H]4O[C@H](COP(=O)(O)CP(=O)(O)O)[C@@H](O)[C@H]4O)CC3)cc2)nn1)Oc1c(F)cc(F)cc1F] |
| Int-206 | 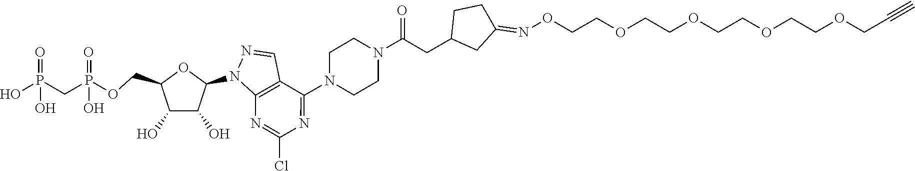 [SMILES: C#CCOCCOCCOCCOCCO/N=C1\CCC(CC(=O)N2CCN(c3nc(Cl)nc4c3cnn4[C@@H]3O[C@H](COP(=O)(O)CP(=O)(O)O)[C@@H](O)[C@H]3O)CC2)C1] |
| Int-207 | 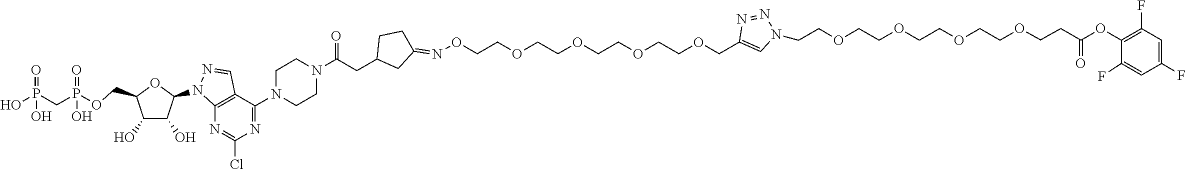 [SMILES: O=C(CCOCCOCCOCCOCCn1cc(COCCOCCOCCOCCO/N=C2\CCC(CC(=O)N3CCN(c4nc(Cl)nc5c4cnn5[C@@H]4O[C@H](COP(=O)(O)CP(=O)(O)O)[C@@H](O)[C@H]4O)CC3)C2)nn1)Oc1c(F)cc(F)cc1F] |
| Int-208 | 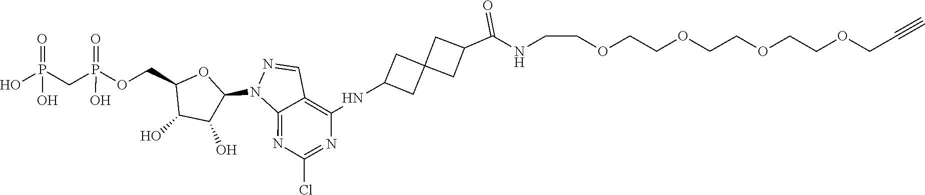 [SMILES: C#CCOCCOCCOCCOCCNC(=O)C1CC2(CC(Nc3nc(Cl)nc4c3cnn4[C@@H]3O[C@H](COP(=O)(O)CP(=O)(O)O)[C@@H](O)[C@H]3O)C2)C1] |
| Int-209 | 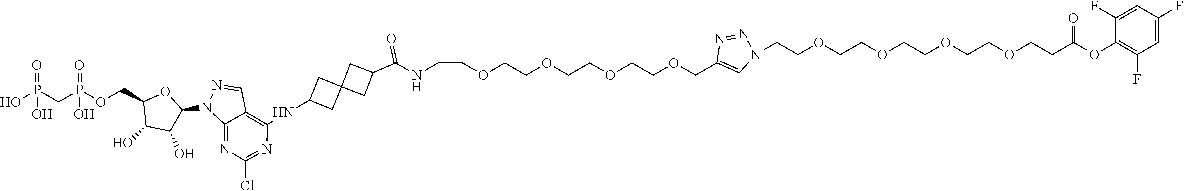 [SMILES: O=C(CCOCCOCCOCCOCCn1cc(COCCOCCOCCOCCNC(=O)C2CC3(CC(Nc4nc(Cl)nc5c4cnn5[C@@H]4O[C@H](COP(=O)(O)CP(=O)(O)O)[C@@H](O)[C@H]4O)C3)C2)nn1)Oc1c(F)cc(F)cc1F] |
| Int-210 | 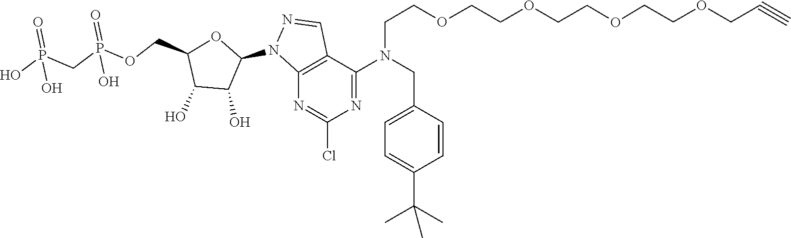 [SMILES: C#CCOCCOCCOCCOCCN(Cc1ccc(C(C)(C)C)cc1)c1nc(Cl)nc2c1cnn2[C@@H]1O[C@H](COP(=O)(O)CP(=O)(O)O)[C@@H](O)[C@H]1O] |
| Int-211 | 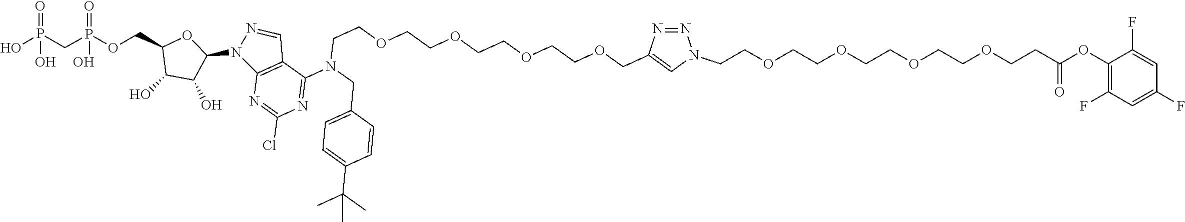 [SMILES: CC(C)(C)c1ccc(CN(CCOCCOCCOCCOCc2cn(CCOCCOCCOCCOCCC(=O)Oc3c(F)cc(F)cc3F)nn2)c2nc(Cl)nc3c2cnn3[C@@H]2O[C@H](COP(=O)(O)CP(=O)(O)O)[C@@H](O)[C@H]2O)cc1] |
| Int-212 | 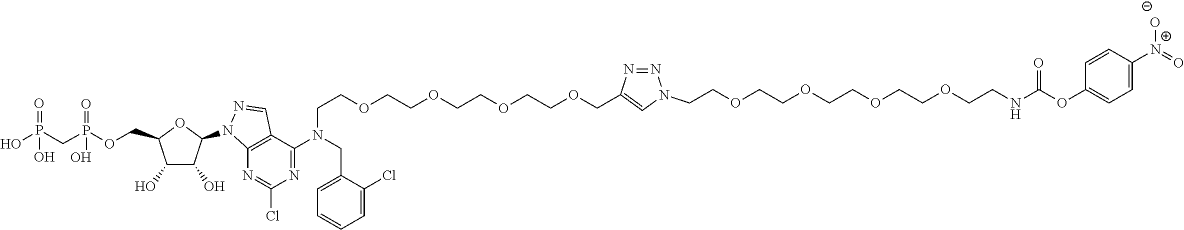 [SMILES: O=C(NCCOCCOCCOCCOCCn1cc(COCCOCCOCCOCCN(Cc2ccccc2Cl)c2nc(Cl)nc3c2cnn3[C@@H]2O[C@H](COP(=O)(O)CP(=O)(O)O)[C@@H](O)[C@H]2O)nn1)Oc1ccc([N+](=O)[O-])cc1] |
| Int-213 | 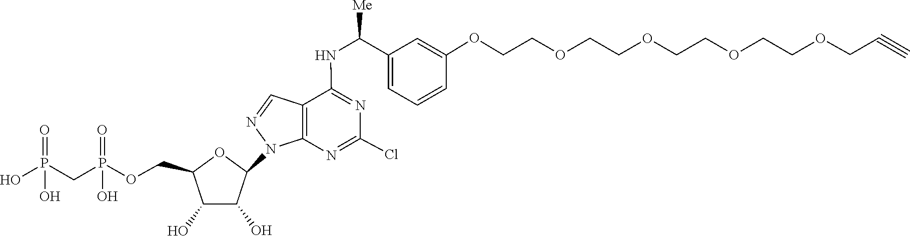 [SMILES: C#CCOCCOCCOCCOCCOc1cccc([C@H](C)Nc2nc(Cl)nc3c2cnn3[C@@H]2O[C@H](COP(=O)(O)CP(=O)(O)O)[C@@H](O)[C@H]2O)c1] |
| Int-214 | 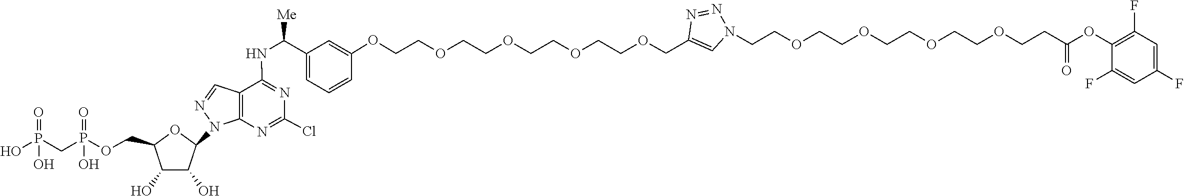 [SMILES: C[C@H](Nc1nc(Cl)nc2c1cnn2[C@@H]1O[C@H](COP(=O)(O)CP(=O)(O)O)[C@@H](O)[C@H]1O)c1cccc(OCCOCCOCCOCCOCc2cn(CCOCCOCCOCCOCCC(=O)Oc3c(F)cc(F)cc3F)nn2)c1] |
| Int-215 | 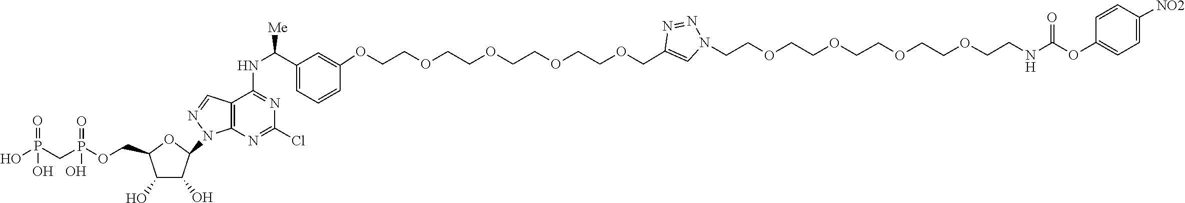 [SMILES: C[C@H](Nc1nc(Cl)nc2c1cnn2[C@@H]1O[C@H](COP(=O)(O)CP(=O)(O)O)[C@@H](O)[C@H]1O)c1cccc(OCCOCCOCCOCCOCc2cn(CCOCCOCCOCCOCCNC(=O)Oc3ccc([N+](=O)[O-])cc3)nn2)c1] |
| Int-216 | 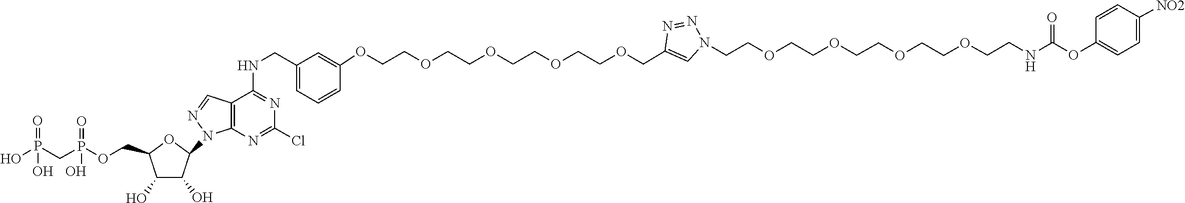 [SMILES: O=C(NCCOCCOCCOCCOCCn1cc(COCCOCCOCCOCCOc2cccc(CNc3nc(Cl)nc4c3cnn4[C@@H]3O[C@H](COP(=O)(O)CP(=O)(O)O)[C@@H](O)[C@H]3O)c2)nn1)Oc1ccc([N+](=O)[O-])cc1] |
| Int-217 | 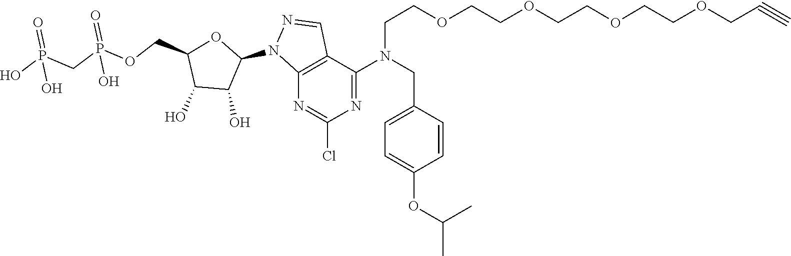 [SMILES: C#CCOCCOCCOCCOCCN(Cc1ccc(OC(C)C)cc1)c1nc(Cl)nc2c1cnn2[C@@H]1O[C@H](COP(=O)(O)CP(=O)(O)O)[C@@H](O)[C@H]1O] |
| Int-218 | 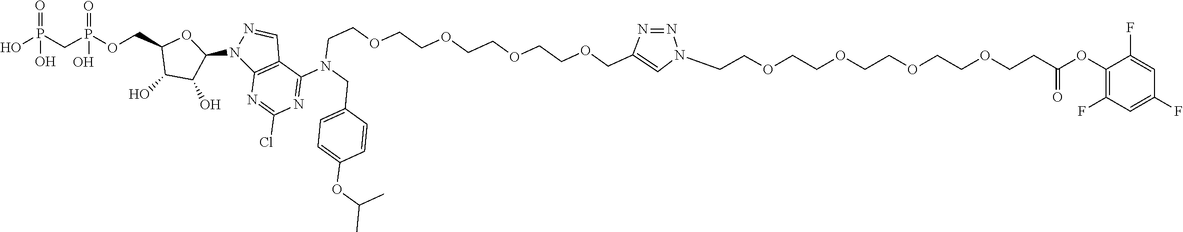 [SMILES: CC(C)Oc1ccc(CN(CCOCCOCCOCCOCc2cn(CCOCCOCCOCCOCCC(=O)Oc3c(F)cc(F)cc3F)nn2)c2nc(Cl)nc3c2cnn3[C@@H]2O[C@H](COP(=O)(O)CP(=O)(O)O)[C@@H](O)[C@H]2O)cc1] |
| Int-219 | 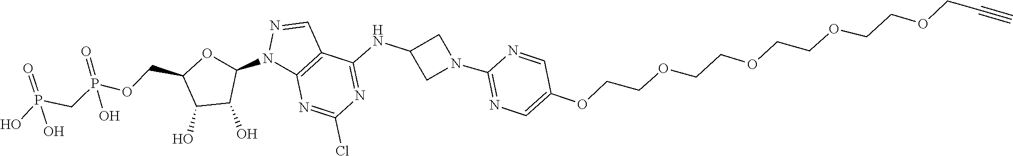 [SMILES: C#CCOCCOCCOCCOCCOc1cnc(N2CC(Nc3nc(Cl)nc4c3cnn4[C@@H]3O[C@H](COP(=O)(O)CP(=O)(O)O)[C@@H](O)[C@H]3O)C2)nc1] |
| Int-220 | 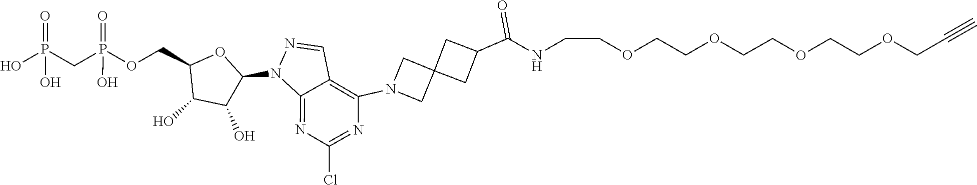 [SMILES: C#CCOCCOCCOCCOCCNC(=O)C1CC2(C1)CN(c1nc(Cl)nc3c1cnn3[C@@H]1O[C@H](COP(=O)(O)CP(=O)(O)O)[C@@H](O)[C@H]1O)C2] |
| Int-221 | 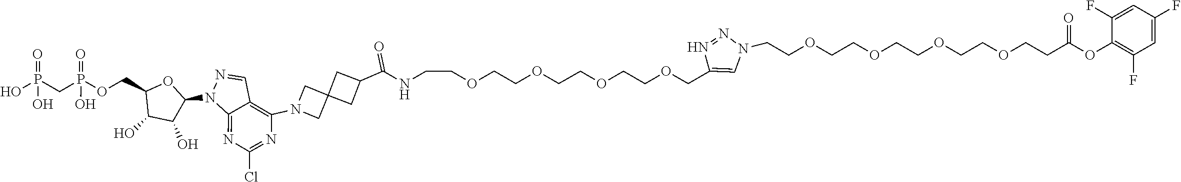 [SMILES: O=C(CCOCCOCCOCCOCCn1cc(COCCOCCOCCOCCNC(=O)C2CC3(C2)CN(c2nc(Cl)nc4c2cnn4[C@@H]2O[C@H](COP(=O)(O)CP(=O)(O)O)[C@@H](O)[C@H]2O)C3)nn1)Oc1c(F)cc(F)cc1F] |
| Int-222 | 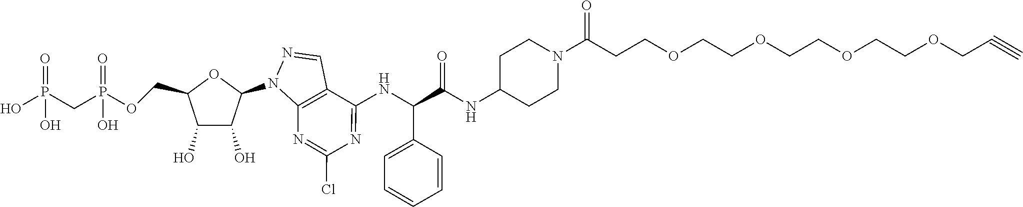 [SMILES: C#CCOCCOCCOCCOCCC(=O)N1CCC(NC(=O)[C@H](Nc2nc(Cl)nc3c2cnn3[C@@H]2O[C@H](COP(=O)(O)CP(=O)(O)O)[C@@H](O)[C@H]2O)c2ccccc2)CC1] |
| Int-223 | 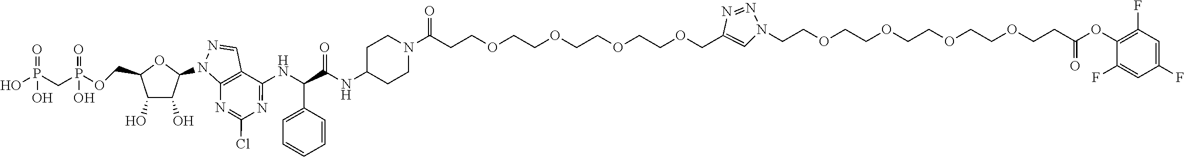 [SMILES: O=C(CCOCCOCCOCCOCCn1cc(COCCOCCOCCOCCC(=O)N2CCC(NC(=O)[C@H](Nc3nc(Cl)nc4c3cnn4[C@@H]3O[C@H](COP(=O)(O)CP(=O)(O)O)[C@@H](O)[C@H]3O)c3ccccc3)CC2)nn1)Oc1c(F)cc(F)cc1F] |
| Int-224 | 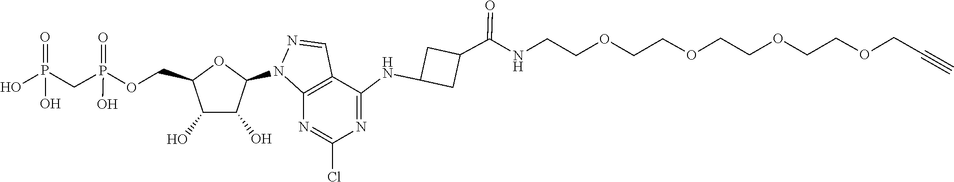 [SMILES: C#CCOCCOCCOCCOCCNC(=O)C1CC(Nc2nc(Cl)nc3c2cnn3[C@@H]2O[C@H](COP(=O)(O)CP(=O)(O)O)[C@@H](O)[C@H]2O)C1] |
| Int-225 | 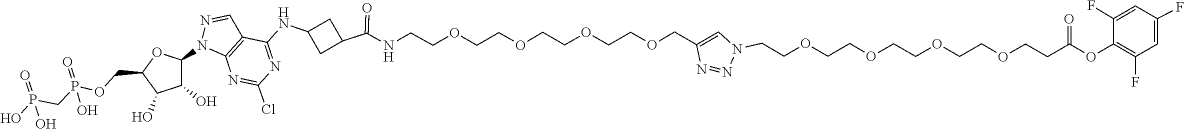 [SMILES: O=C(CCOCCOCCOCCOCCn1cc(COCCOCCOCCOCCNC(=O)C2CC(Nc3nc(Cl)nc4c3cnn4[C@@H]3O[C@H](COP(=O)(O)CP(=O)(O)O)[C@@H](O)[C@H]3O)C2)nn1)Oc1c(F)cc(F)cc1F] |
| Int-226 | 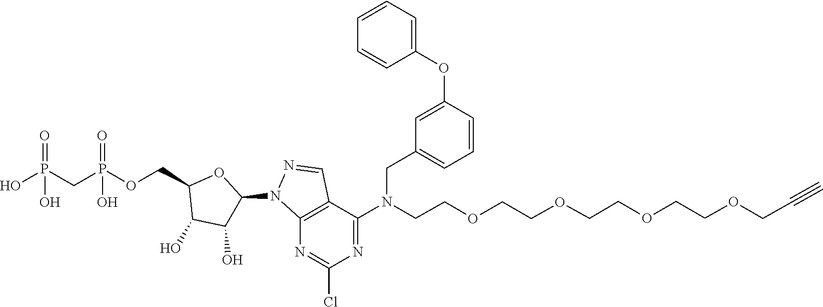 [SMILES: C#CCOCCOCCOCCOCCN(Cc1cccc(Oc2ccccc2)c1)c1nc(Cl)nc2c1cnn2[C@@H]1O[C@H](COP(=O)(O)CP(=O)(O)O)[C@@H](O)[C@H]1O] |
| Int-227 | 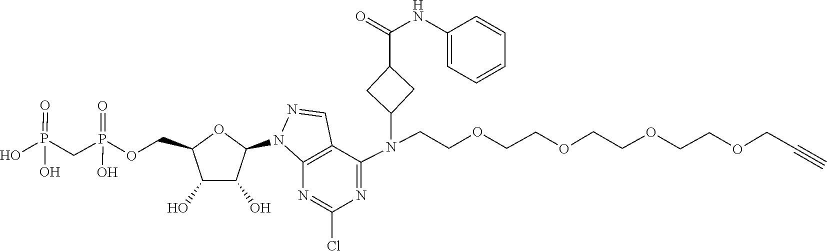 [SMILES: C#CCOCCOCCOCCOCCN(c1nc(Cl)nc2c1cnn2[C@@H]1O[C@H](COP(=O)(O)CP(=O)(O)O)[C@@H](O)[C@H]1O)C1CC(C(=O)Nc2ccccc2)C1] |
| Int-228 | 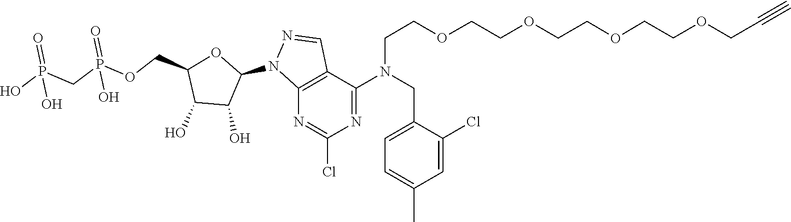 [SMILES: C#CCOCCOCCOCCOCCN(Cc1ccc(C)cc1Cl)c1nc(Cl)nc2c1cnn2[C@@H]1O[C@H](COP(=O)(O)CP(=O)(O)O)[C@@H](O)[C@H]1O] |
| Int-229 | 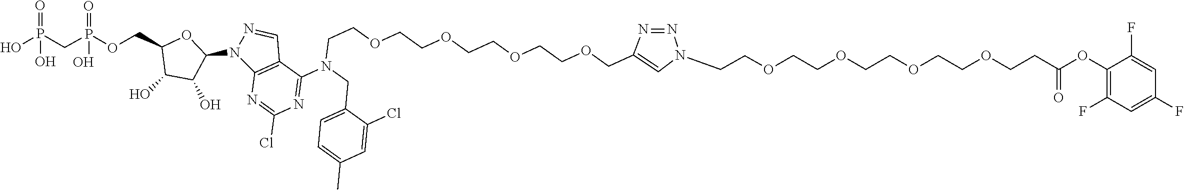 [SMILES: Cc1ccc(CN(CCOCCOCCOCCOCc2cn(CCOCCOCCOCCOCCC(=O)Oc3c(F)cc(F)cc3F)nn2)c2nc(Cl)nc3c2cnn3[C@@H]2O[C@H](COP(=O)(O)CP(=O)(O)O)[C@@H](O)[C@H]2O)c(Cl)c1] |
| Int-230 | 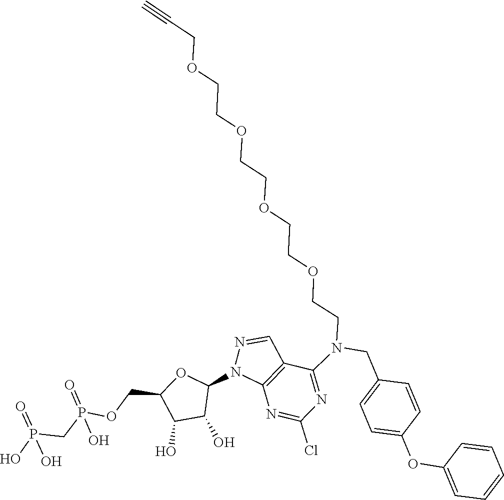 [SMILES: C#CCOCCOCCOCCOCCN(Cc1ccc(Oc2ccccc2)cc1)c1nc(Cl)nc2c1cnn2[C@@H]1O[C@H](COP(=O)(O)CP(=O)(O)O)[C@@H](O)[C@H]1O] |
| Int-231 | 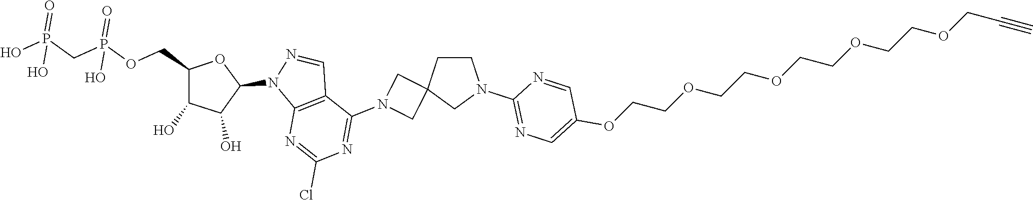 [SMILES: C#CCOCCOCCOCCOCCOc1cnc(N2CCC3(C2)CN(c2nc(Cl)nc4c2cnn4[C@@H]2O[C@H](COP(=O)(O)CP(=O)(O)O)[C@@H](O)[C@H]2O)C3)nc1] |
| Int-232 | 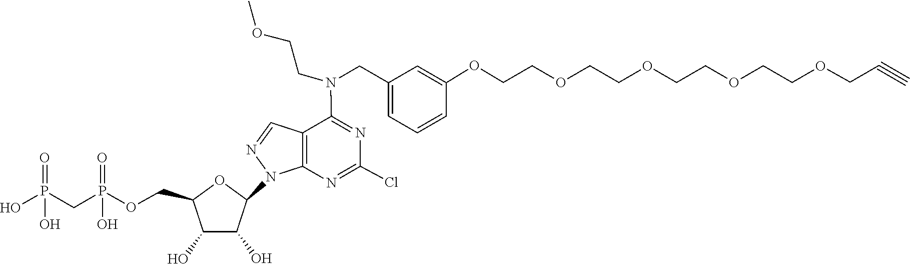 [SMILES: C#CCOCCOCCOCCOCCOc1cccc(CN(CCOC)c2nc(Cl)nc3c2cnn3[C@@H]2O[C@H](COP(=O)(O)CP(=O)(O)O)[C@@H](O)[C@H]2O)c1] |
| Int-233 | 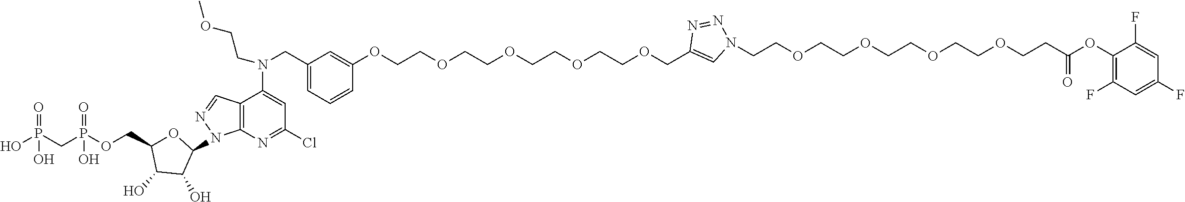 [SMILES: COCCN(Cc1cccc(OCCOCCOCCOCCOCc2cn(CCOCCOCCOCCOCCC(=O)Oc3c(F)cc(F)cc3F)nn2)c1)c1cc(Cl)nc2c1cnn2[C@@H]1O[C@H](COP(=O)(O)CP(=O)(O)O)[C@@H](O)[C@H]1O] |
| Int-234 | 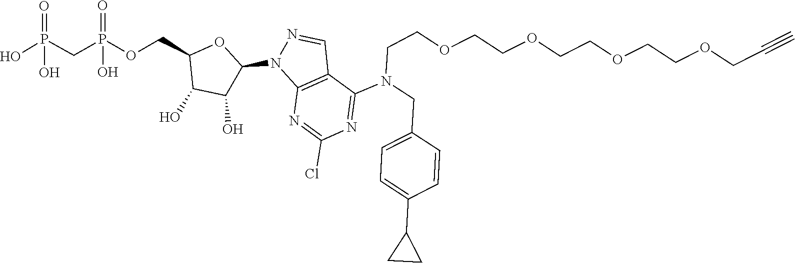 [SMILES: C#CCOCCOCCOCCOCCN(Cc1ccc(C2CC2)cc1)c1nc(Cl)nc2c1cnn2[C@@H]1O[C@H](COP(=O)(O)CP(=O)(O)O)[C@@H](O)[C@H]1O] |
| Int-235 | 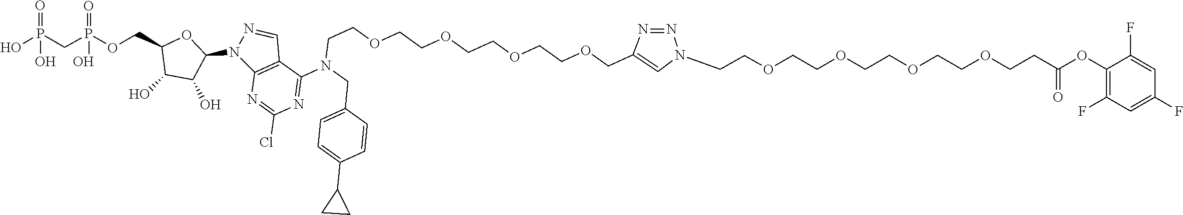 [SMILES: O=C(CCOCCOCCOCCOCCn1cc(COCCOCCOCCOCCN(Cc2ccc(C3CC3)cc2)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-236 | 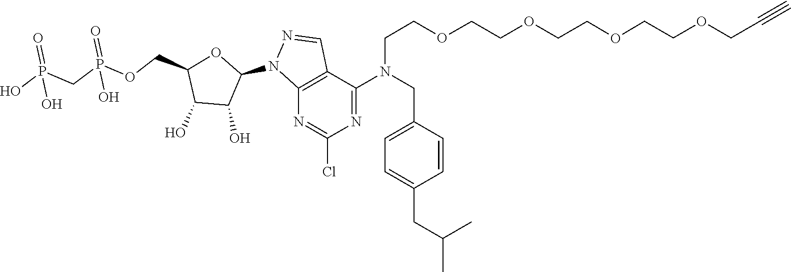 [SMILES: C#CCOCCOCCOCCOCCN(Cc1ccc(CC(C)C)cc1)c1nc(Cl)nc2c1cnn2[C@@H]1O[C@H](COP(=O)(O)CP(=O)(O)O)[C@@H](O)[C@H]1O] |
| Int-237 | 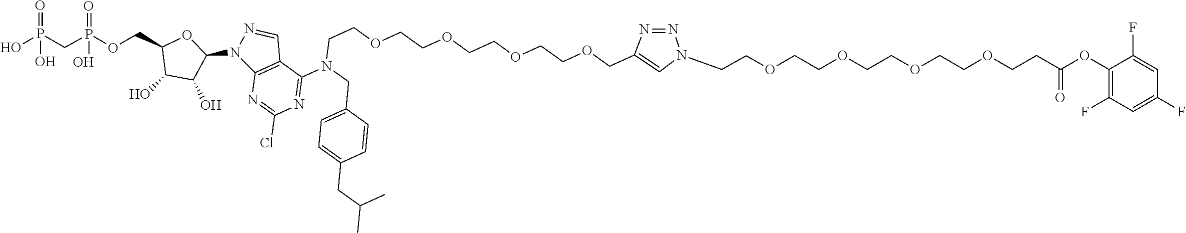 [SMILES: CC(C)Cc1ccc(CN(CCOCCOCCOCCOCc2cn(CCOCCOCCOCCOCCC(=O)Oc3c(F)cc(F)cc3F)nn2)c2nc(Cl)nc3c2cnn3[C@@H]2O[C@H](COP(=O)(O)CP(=O)(O)O)[C@@H](O)[C@H]2O)cc1] |
| Int-238 | 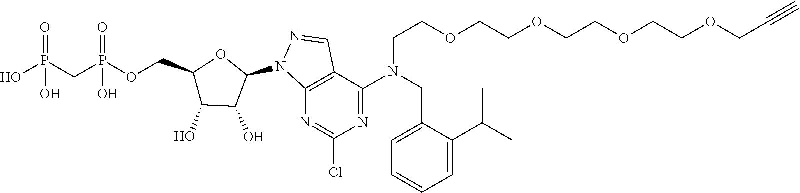 [SMILES: C#CCOCCOCCOCCOCCN(Cc1ccccc1C(C)C)c1nc(Cl)nc2c1cnn2[C@@H]1O[C@H](COP(=O)(O)CP(=O)(O)O)[C@@H](O)[C@H]1O] |
| Int-239 | 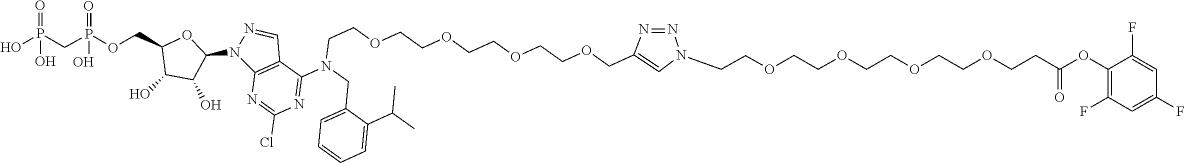 [SMILES: CC(C)c1ccccc1CN(CCOCCOCCOCCOCc1cn(CCOCCOCCOCCOCCC(=O)Oc2c(F)cc(F)cc2F)nn1)c1nc(Cl)nc2c1cnn2[C@@H]1O[C@H](COP(=O)(O)CP(=O)(O)O)[C@@H](O)[C@H]1O] |
| Int-240 | 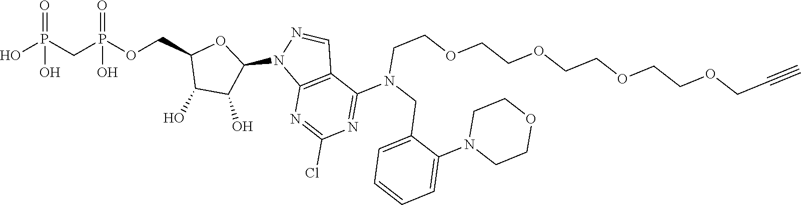 [SMILES: C#CCOCCOCCOCCOCCN(Cc1ccccc1N1CCOCC1)c1nc(Cl)nc2c1cnn2[C@@H]1O[C@H](COP(=O)(O)CP(=O)(O)O)[C@@H](O)[C@H]1O] |
| Int-241 | 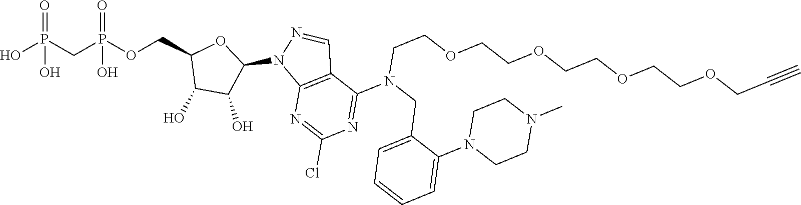 [SMILES: C#CCOCCOCCOCCOCCN(Cc1ccccc1N1CCN(C)CC1)c1nc(Cl)nc2c1cnn2[C@@H]1O[C@H](COP(=O)(O)CP(=O)(O)O)[C@@H](O)[C@H]1O] |
| Int-242 | 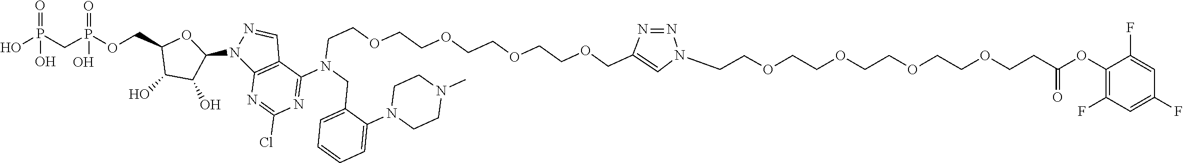 [SMILES: CN1CCN(c2ccccc2CN(CCOCCOCCOCCOCc2cn(CCOCCOCCOCCOCCC(=O)Oc3c(F)cc(F)cc3F)nn2)c2nc(Cl)nc3c2cnn3[C@@H]2O[C@H](COP(=O)(O)CP(=O)(O)O)[C@@H](O)[C@H]2O)CC1] |
| Int-243 | 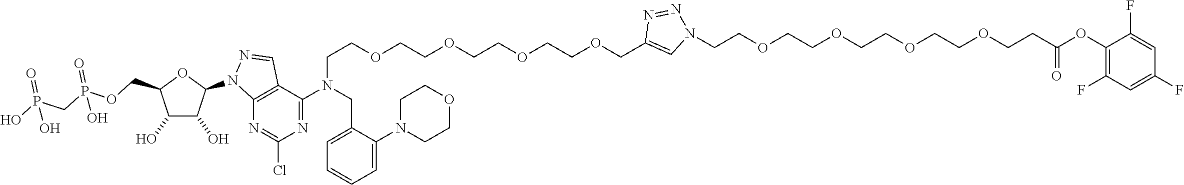 [SMILES: O=C(CCOCCOCCOCCOCCn1cc(COCCOCCOCCOCCN(Cc2ccccc2N2CCOCC2)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-244 | 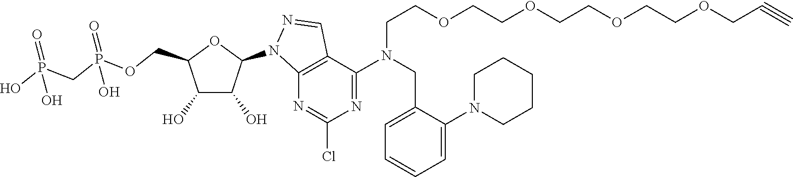 [SMILES: C#CCOCCOCCOCCOCCN(Cc1ccccc1N1CCCCC1)c1nc(Cl)nc2c1cnn2[C@@H]1O[C@H](COP(=O)(O)CP(=O)(O)O)[C@@H](O)[C@H]1O] |
| Int-245 | 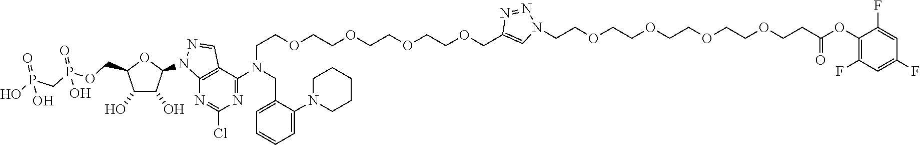 [SMILES: O=C(CCOCCOCCOCCOCCn1cc(COCCOCCOCCOCCN(Cc2ccccc2N2CCCCC2)c2nc(Cl)nc3c2cnn3[C@@H]2O[C@H](COP(=O)(O)CP(=O)(O)O)[C@@H](O)[C@H]2O)nn1)Oc1c(F)cc(F)cc1F] |
| Int-246 | 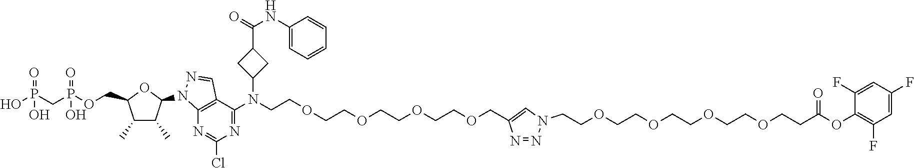 [SMILES: C[C@@H]1[C@H](C)[C@@H](COP(=O)(O)CP(=O)(O)O)O[C@H]1n1ncc2c(N(CCOCCOCCOCCOCc3cn(CCOCCOCCOCCOCCC(=O)Oc4c(F)cc(F)cc4F)nn3)C3CC(C(=O)Nc4ccccc4)C3)nc(Cl)nc21] |
| Int-247 | 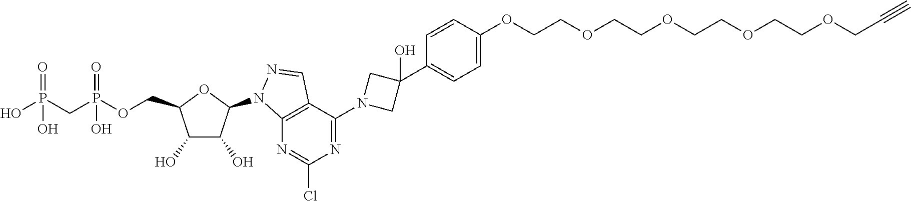 [SMILES: C#CCOCCOCCOCCOCCOc1ccc(C2(O)CN(c3nc(Cl)nc4c3cnn4[C@@H]3O[C@H](COP(=O)(O)CP(=O)(O)O)[C@@H](O)[C@H]3O)C2)cc1] |
| Int-248 | 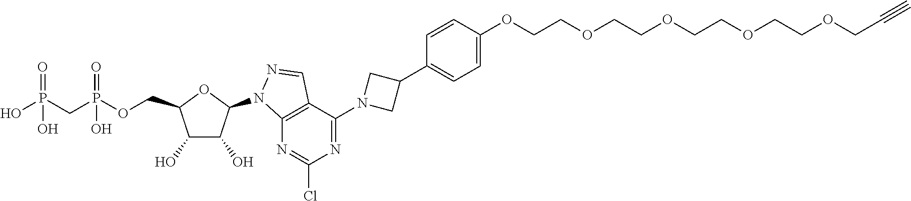 [SMILES: C#CCOCCOCCOCCOCCOc1ccc(C2CN(c3nc(Cl)nc4c3cnn4[C@@H]3O[C@H](COP(=O)(O)CP(=O)(O)O)[C@@H](O)[C@H]3O)C2)cc1] |
| Int-249 | 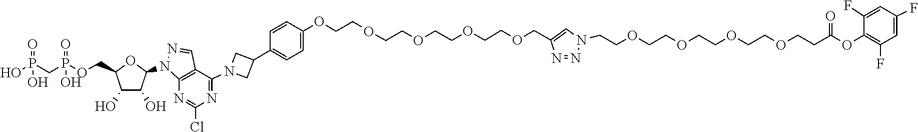 [SMILES: O=C(CCOCCOCCOCCOCCn1cc(COCCOCCOCCOCCOc2ccc(C3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F] |
| Int-250 | 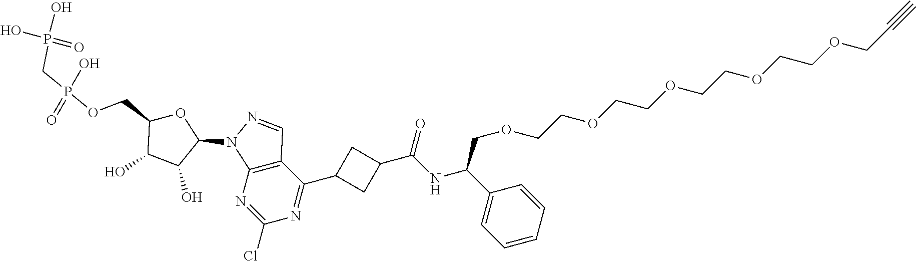 [SMILES: C#CCOCCOCCOCCOCCOC[C@H](NC(=O)C1CC(c2nc(Cl)nc3c2cnn3[C@@H]2O[C@H](COP(=O)(O)CP(=O)(O)O)[C@@H](O)[C@H]2O)C1)c1ccccc1] |
| Int-251 | 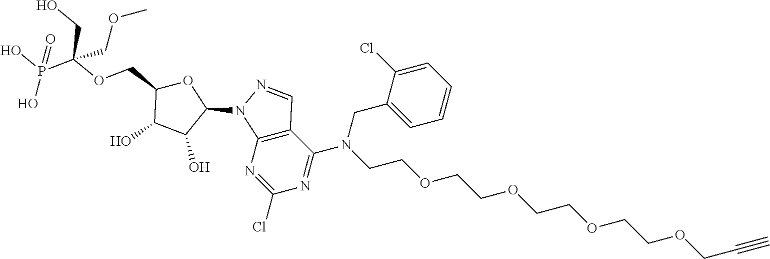 [SMILES: C#CCOCCOCCOCCOCCN(Cc1ccccc1Cl)c1nc(Cl)nc2c1cnn2[C@@H]1O[C@H](CO[C@@](CO)(COC)P(=O)(O)O)[C@@H](O)[C@H]1O] |
| Int-252 | 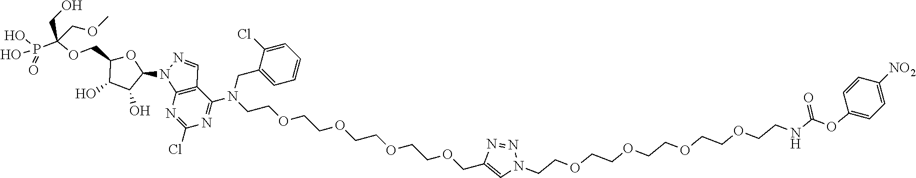 [SMILES: COC[C@](CO)(OC[C@H]1O[C@@H](n2ncc3c(N(CCOCCOCCOCCOCc4cn(CCOCCOCCOCCOCCNC(=O)Oc5ccc([N+](=O)[O-])cc5)nn4)Cc4ccccc4Cl)nc(Cl)nc32)[C@H](O)[C@@H]1O)P(=O)(O)O] |
| Int-253 | 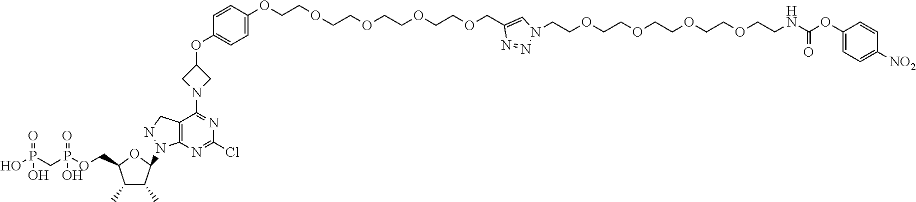 [SMILES: C[C@@H]1[C@H](C)[C@@H](COP(=O)(O)CP(=O)(O)O)O[C@H]1N1NCc2c(N3CC(Oc4ccc(OCCOCCOCCOCCOCc5cn(CCOCCOCCOCCOCCNC(=O)Oc6ccc([N+](=O)[O-])cc6)nn5)cc4)C3)nc(Cl)nc21] |
| Int-254 | 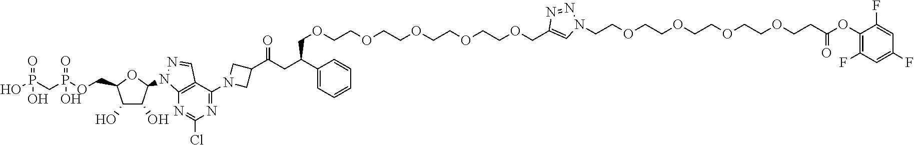 [SMILES: O=C(CCOCCOCCOCCOCCn1cc(COCCOCCOCCOCCOC[C@H](CC(=O)C2CN(c3nc(Cl)nc4c3cnn4[C@@H]3O[C@H](COP(=O)(O)CP(=O)(O)O)[C@@H](O)[C@H]3O)C2)c2ccccc2)nn1)Oc1c(F)cc(F)cc1F] |
| Int-255 | 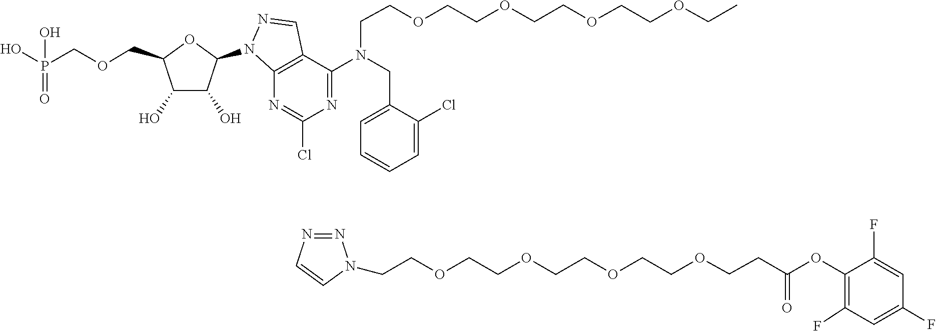 [SMILES: CCOCCOCCOCCOCCN(Cc1ccccc1Cl)c1nc(Cl)nc2c1cnn2[C@@H]1O[C@H](COCP(=O)(O)O)[C@@H](O)[C@H]1O ; O=C(CCOCCOCCOCCOCCn1ccnn1)Oc1c(F)cc(F)cc1F] |
| Int-256 | 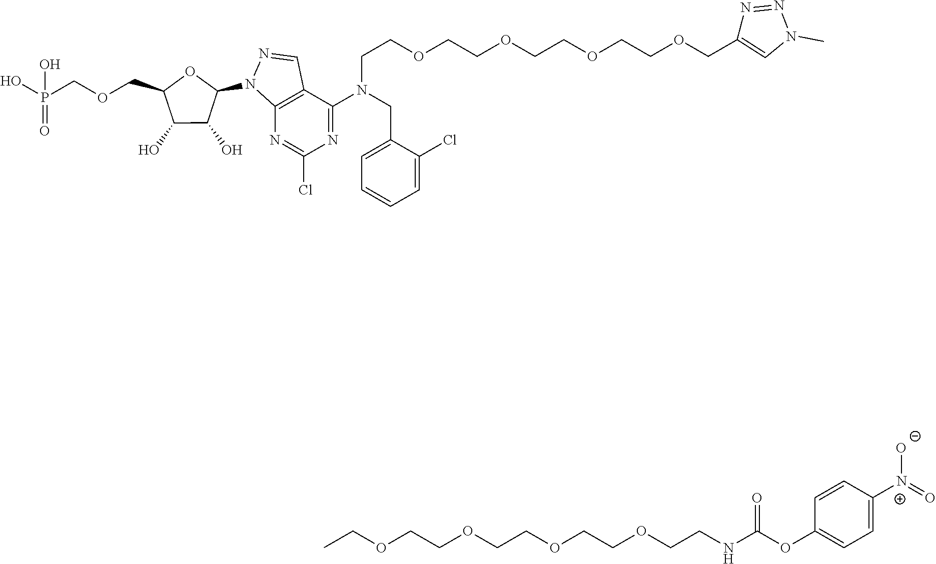 |
| Int-257 | 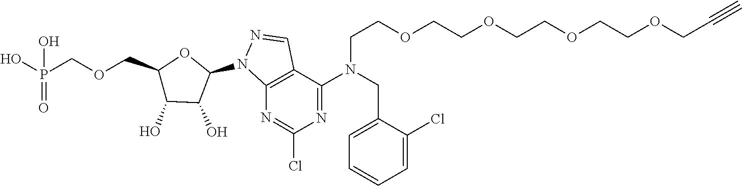 [SMILES: C#CCOCCOCCOCCOCCN(Cc1ccccc1Cl)c1nc(Cl)nc2c1cnn2[C@@H]1O[C@H](COCP(=O)(O)O)[C@@H](O)[C@H]1O] |
| Int-258 | 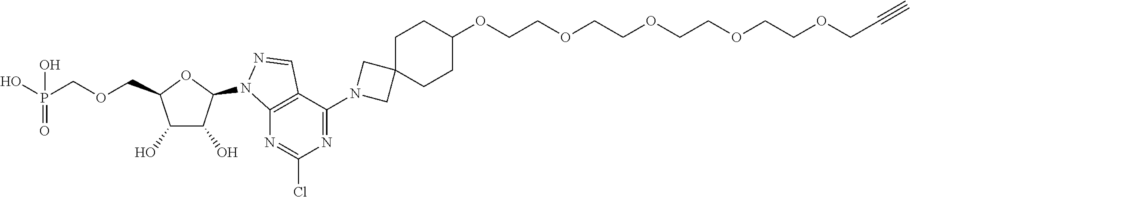 [SMILES: C#CCOCCOCCOCCOCCOC1CCC2(CC1)CN(c1nc(Cl)nc3c1cnn3[C@@H]1O[C@H](COCP(=O)(O)O)[C@@H](O)[C@H]1O)C2] |
| Int-259 | 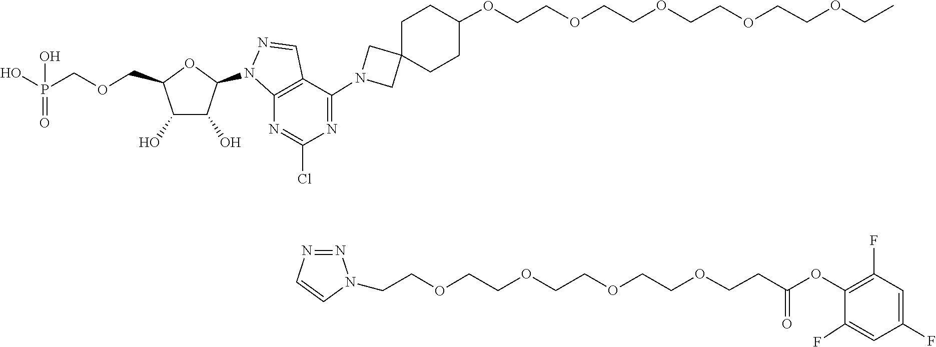 [SMILES: CCOCCOCCOCCOCCOC1CCC2(CC1)CN(c1nc(Cl)nc3c1cnn3[C@@H]1O[C@H](COCP(=O)(O)O)[C@@H](O)[C@H]1O)C2 ; O=C(CCOCCOCCOCCOCCn1ccnn1)Oc1c(F)cc(F)cc1F] |
| Int-260 | 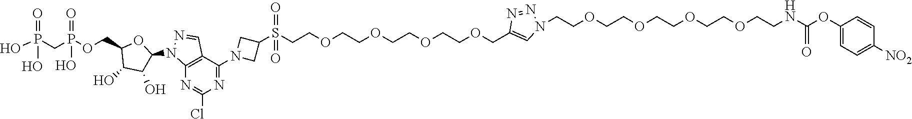 [SMILES: O=C(NCCOCCOCCOCCOCCn1cc(COCCOCCOCCOCCS(=O)(=O)C2CN(c3nc(Cl)nc4c3cnn4[C@@H]3O[C@H](COP(=O)(O)CP(=O)(O)O)[C@@H](O)[C@H]3O)C2)nn1)Oc1ccc([N+](=O)[O-])cc1] |
| Int-261 | 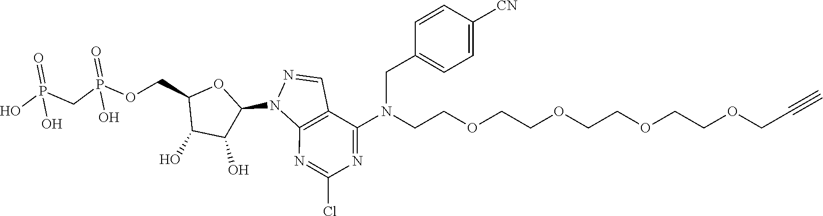 [SMILES: C#CCOCCOCCOCCOCCN(Cc1ccc(C#N)cc1)c1nc(Cl)nc2c1cnn2[C@@H]1O[C@H](COP(=O)(O)CP(=O)(O)O)[C@@H](O)[C@H]1O] |
| Int-262 | 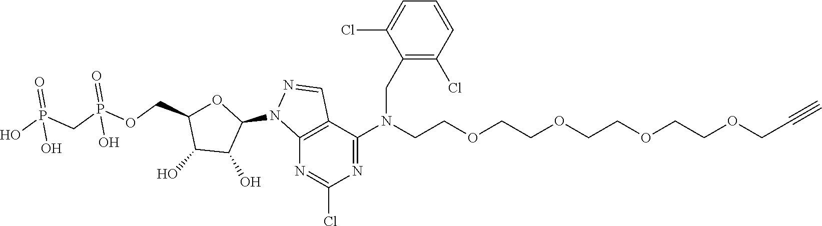 [SMILES: C#CCOCCOCCOCCOCCN(Cc1c(Cl)cccc1Cl)c1nc(Cl)nc2c1cnn2[C@@H]1O[C@H](COP(=O)(O)CP(=O)(O)O)[C@@H](O)[C@H]1O] |
| Int-263 | 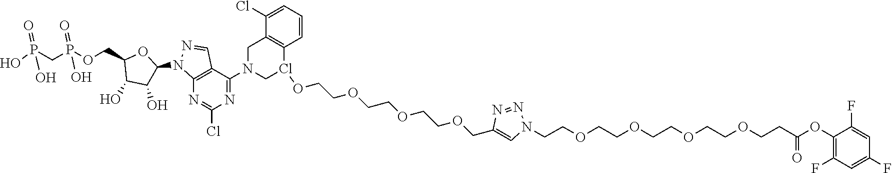 |
| Int-264 | 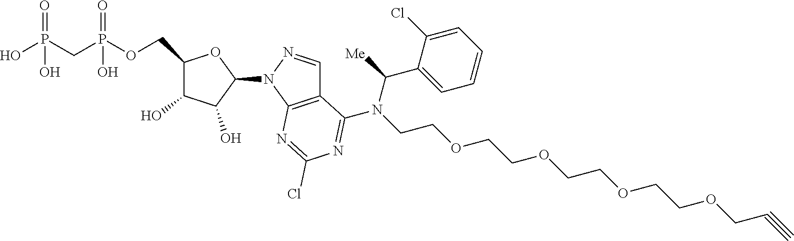 [SMILES: C#CCOCCOCCOCCOCCN(c1nc(Cl)nc2c1cnn2[C@@H]1O[C@H](COP(=O)(O)CP(=O)(O)O)[C@@H](O)[C@H]1O)[C@@H](C)c1ccccc1Cl] |
| Int-265 | 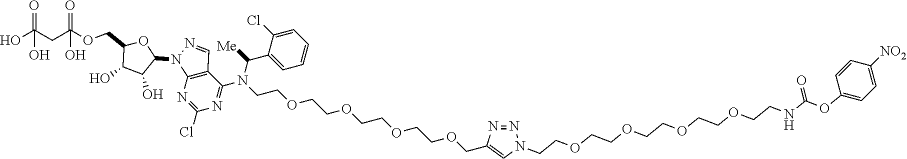 |
| Int-266 | 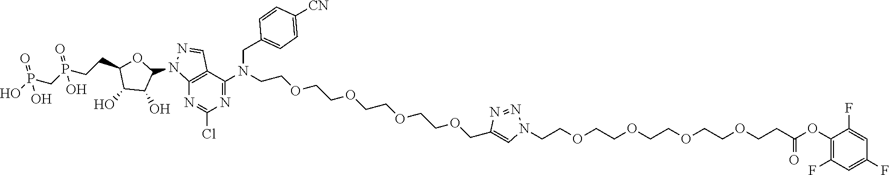 |
| Int-267 | 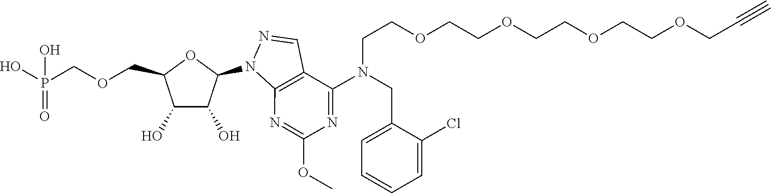 [SMILES: C#CCOCCOCCOCCOCCN(Cc1ccccc1Cl)c1nc(OC)nc2c1cnn2[C@@H]1O[C@H](COCP(=O)(O)O)[C@@H](O)[C@H]1O] |
| Int-268 | 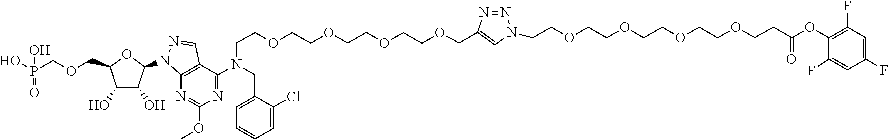 [SMILES: COc1nc(N(CCOCCOCCOCCOCc2cn(CCOCCOCCOCCOCCC(=O)Oc3c(F)cc(F)cc3F)nn2)Cc2ccccc2Cl)c2cnn([C@@H]3O[C@H](COCP(=O)(O)O)[C@@H](O)[C@H]3O)c2n1] |
| Int-269 | 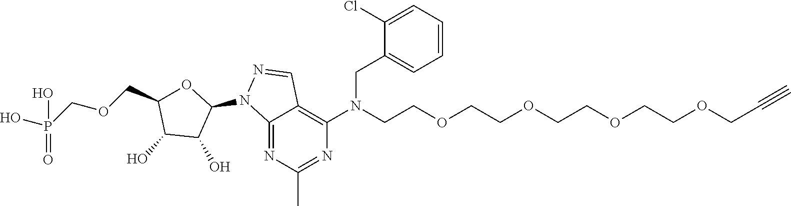 [SMILES: C#CCOCCOCCOCCOCCN(Cc1ccccc1Cl)c1nc(C)nc2c1cnn2[C@@H]1O[C@H](COCP(=O)(O)O)[C@@H](O)[C@H]1O] |
| Int-270 | 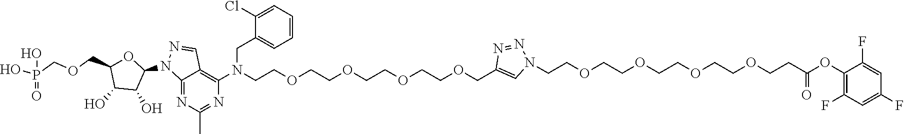 [SMILES: Cc1nc(N(CCOCCOCCOCCOCc2cn(CCOCCOCCOCCOCCC(=O)Oc3c(F)cc(F)cc3F)nn2)Cc2ccccc2Cl)c2cnn([C@@H]3O[C@H](COCP(=O)(O)O)[C@@H](O)[C@H]3O)c2n1] |
| Int-271 | 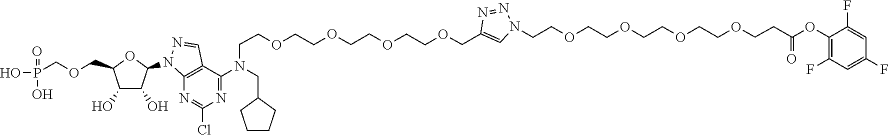 [SMILES: O=C(CCOCCOCCOCCOCCn1cc(COCCOCCOCCOCCN(CC2CCCC2)c2nc(Cl)nc3c2cnn3[C@@H]2O[C@H](COCP(=O)(O)O)[C@@H](O)[C@H]2O)nn1)Oc1c(F)cc(F)cc1F] |
| Int-272 | 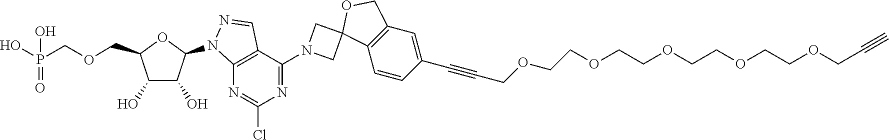 [SMILES: C#CCOCCOCCOCCOCCOCC#Cc1ccc2c(c1)COC21CN(c2nc(Cl)nc3c2cnn3[C@@H]2O[C@H](COCP(=O)(O)O)[C@@H](O)[C@H]2O)C1] |
| Int-273 | 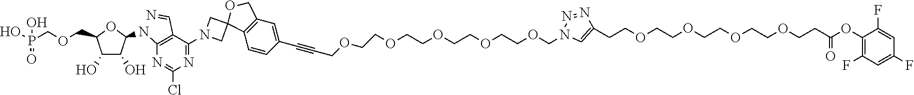 [SMILES: O=C(CCOCCOCCOCCOCCc1cn(COCCOCCOCCOCCOCC#Cc2ccc3c(c2)COC32CN(c3nc(Cl)nc4c3cnn4[C@@H]3O[C@H](COCP(=O)(O)O)[C@@H](O)[C@H]3O)C2)nn1)Oc1c(F)cc(F)cc1F] |
| Int-274 | 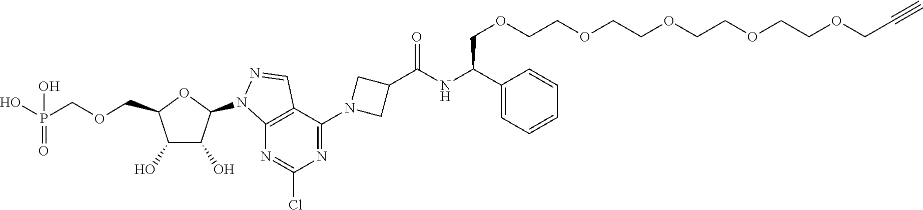 [SMILES: C#CCOCCOCCOCCOCCOC[C@H](NC(=O)C1CN(c2nc(Cl)nc3c2cnn3[C@@H]2O[C@H](COCP(=O)(O)O)[C@@H](O)[C@H]2O)C1)c1ccccc1] |
| Int-275 | 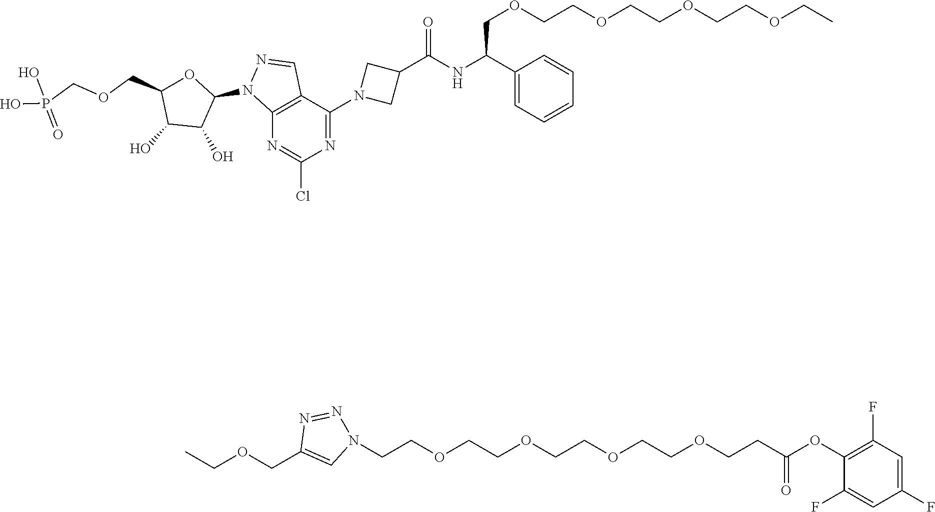 |
| Int-276 | 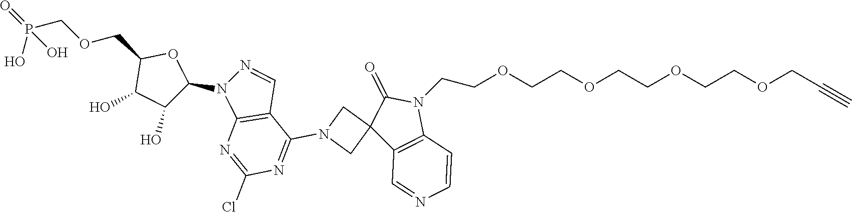 [SMILES: C#CCOCCOCCOCCOCCN1C(=O)C2(CN(c3nc(Cl)nc4c3cnn4[C@@H]3O[C@H](COCP(=O)(O)O)[C@@H](O)[C@H]3O)C2)c2cnccc21] |
| Int-277 | 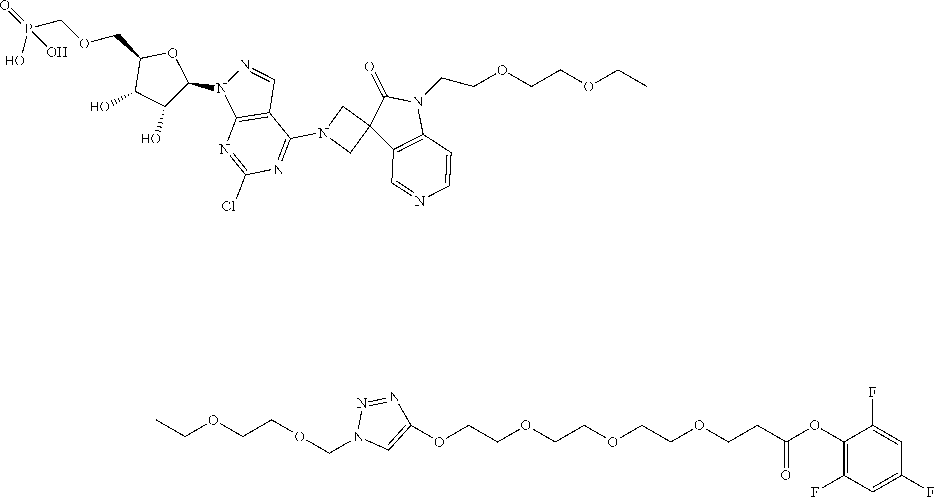 |
| Int-278 | 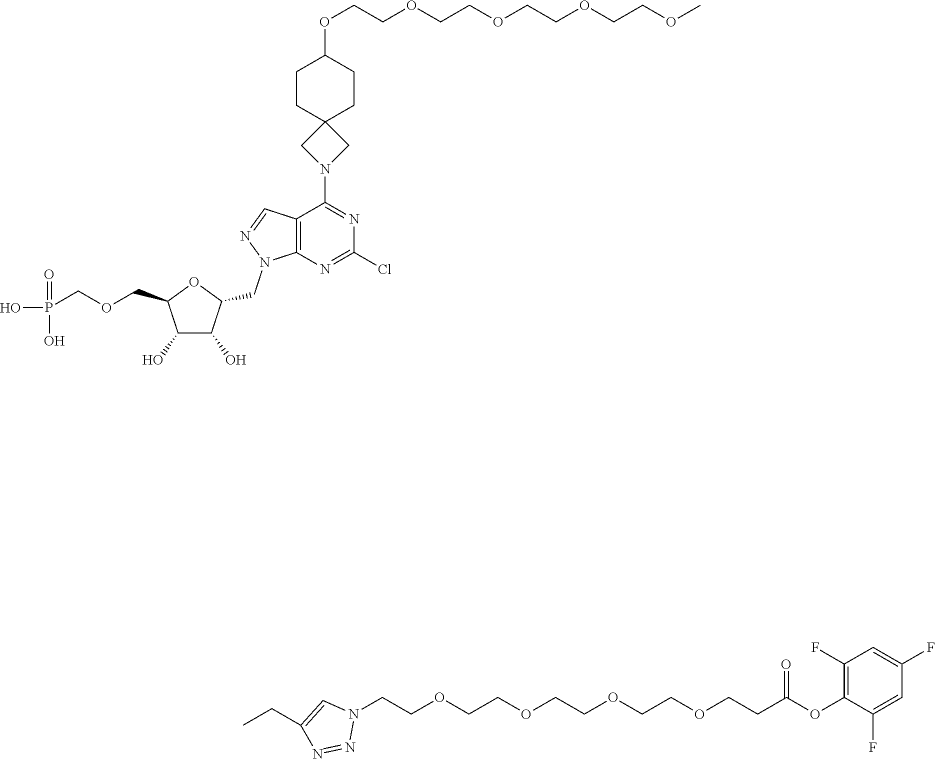 |
| Int-279 | 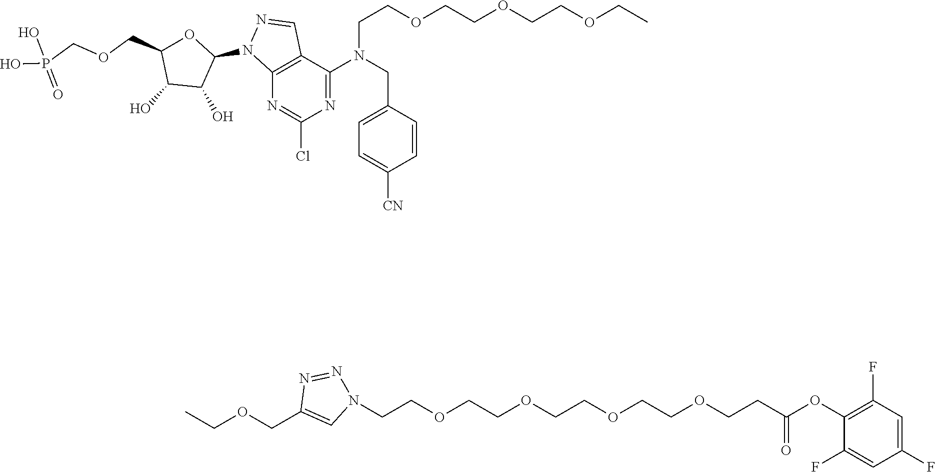 |
| Int-280 | 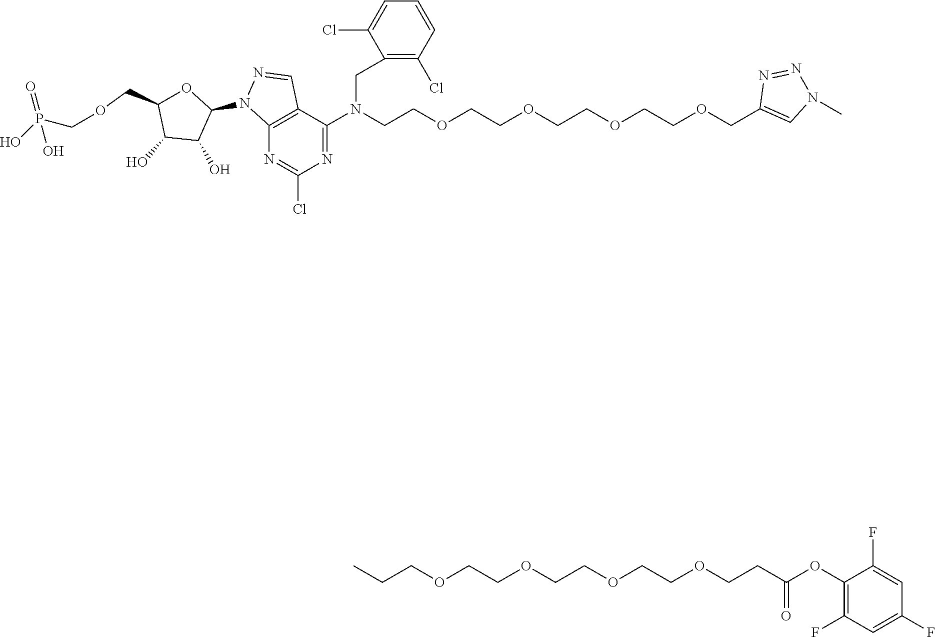 |
| Int-281 | 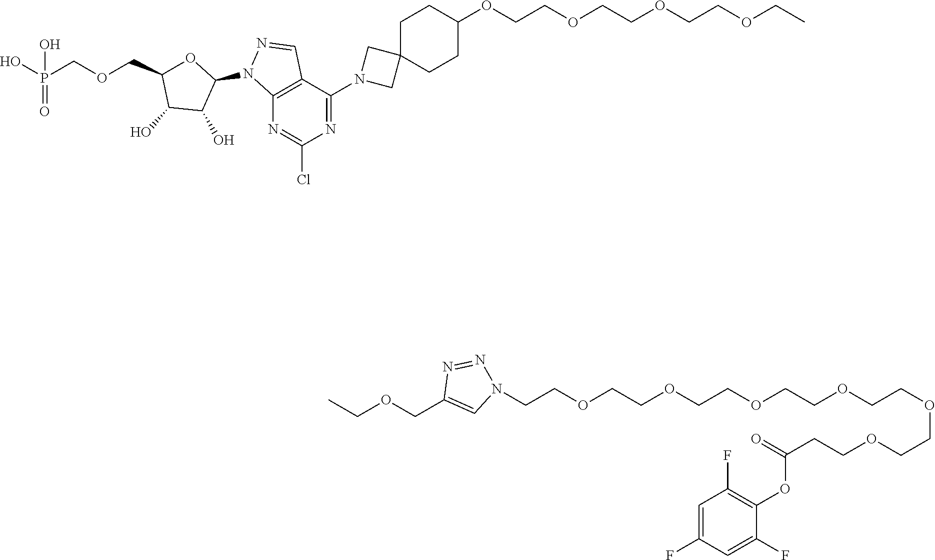 |
| Int-282 | 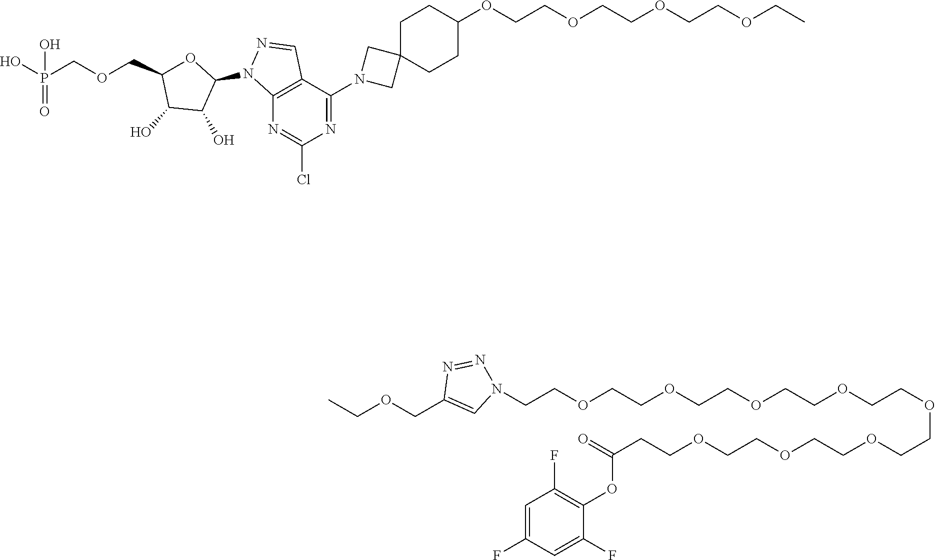 |
| Int-283 | 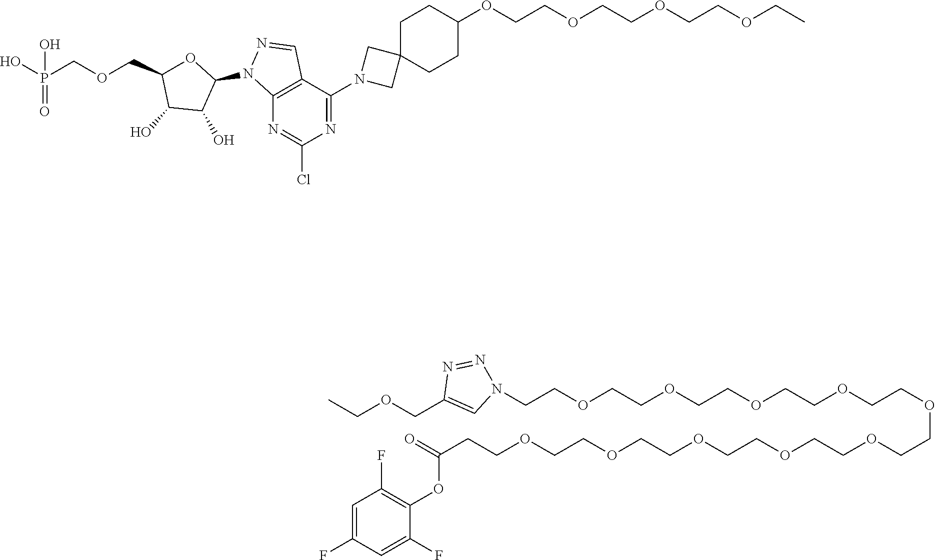 [SMILES: CCOCCOCCOCCOC1CCC2(CC1)CN(c1nc(Cl)nc3c1cnn3[C@@H]1O[C@H](COCP(=O)(O)O)[C@@H](O)[C@H]1O)C2 ; CCOCc1cn(CCOCCOCCOCCOCCOCCOCCOCCOCCOCCOCCC(=O)Oc2c(F)cc(F)cc2F)nn1] |
| Int-284 | 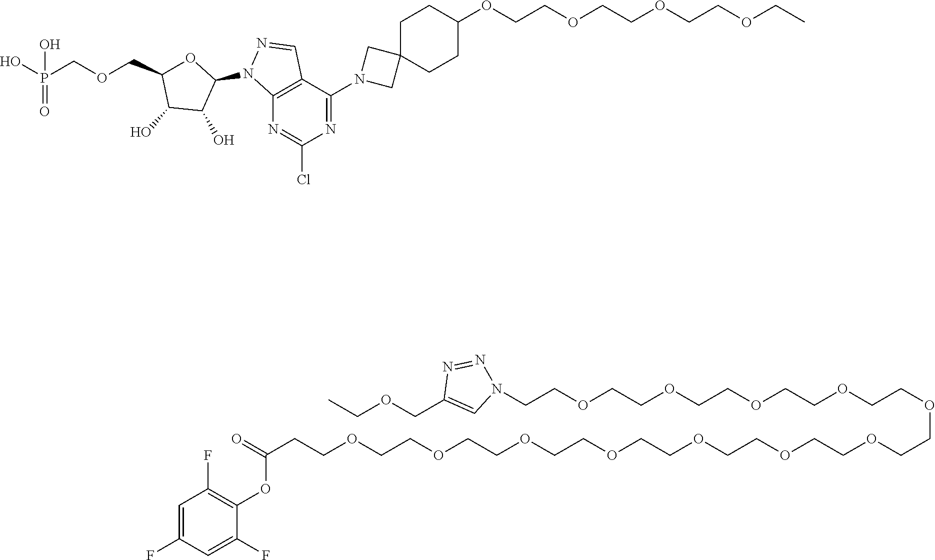 [SMILES: CCOCCOCCOCCOC1CCC2(CC1)CN(c1nc(Cl)nc3c1cnn3[C@@H]1O[C@H](COCP(=O)(O)O)[C@@H](O)[C@H]1O)C2 ; CCOCc1cn(CCOCCOCCOCCOCCOCCOCCOCCOCCOCCOCCOCCOCCC(=O)Oc2c(F)cc(F)cc2F)nn1] |
| Int-285 | 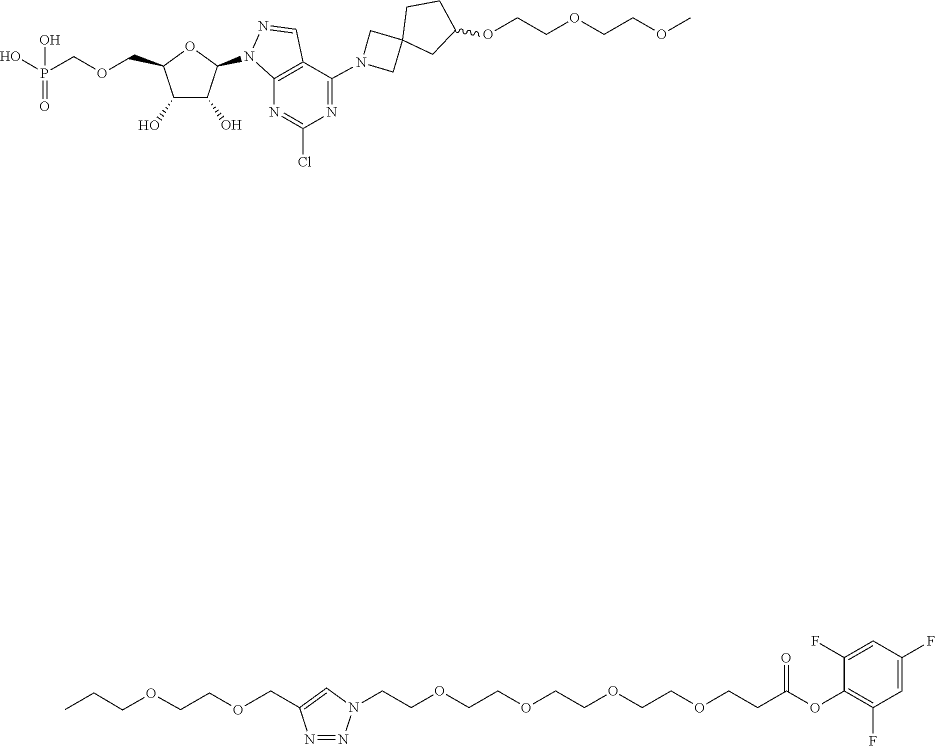 |
| Int-286 | 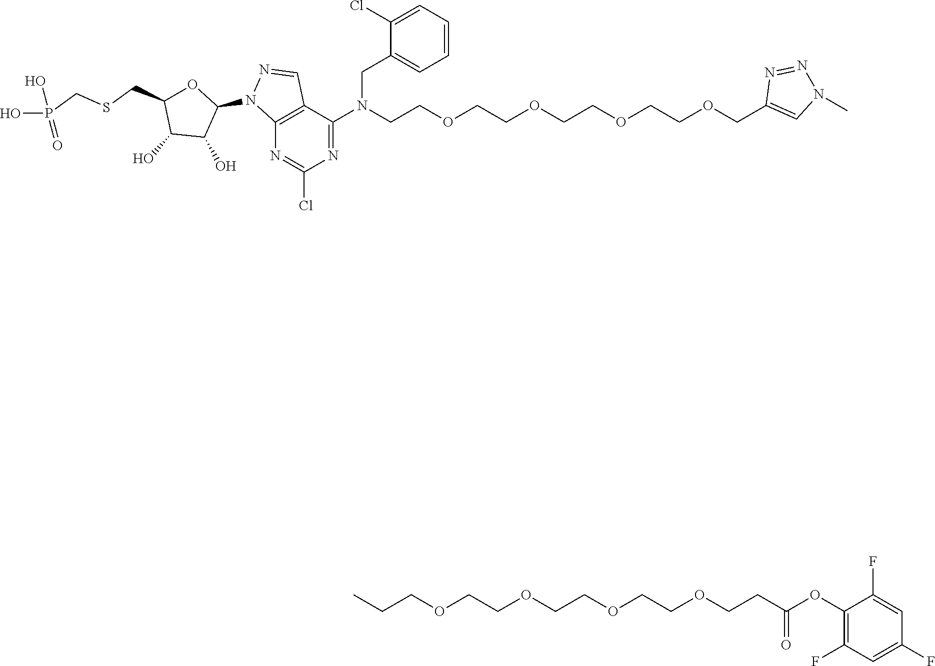 |
| Int-287 | 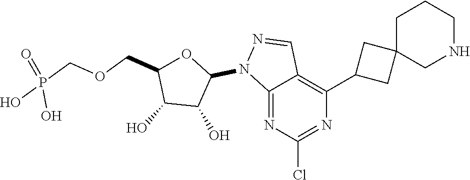 |
| Int-288 | 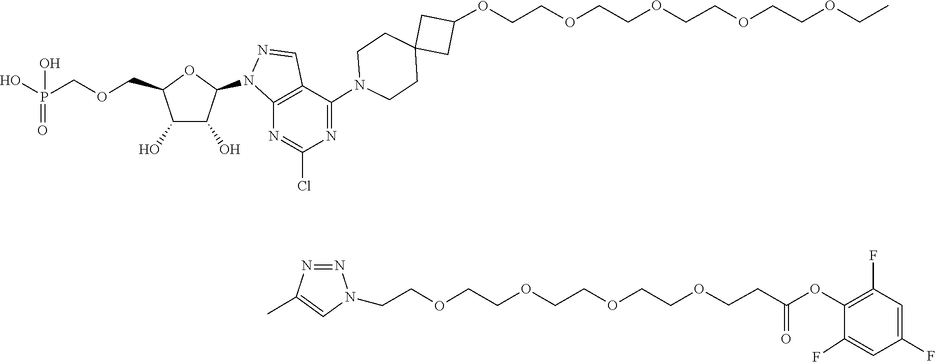 |
| Int-289 | 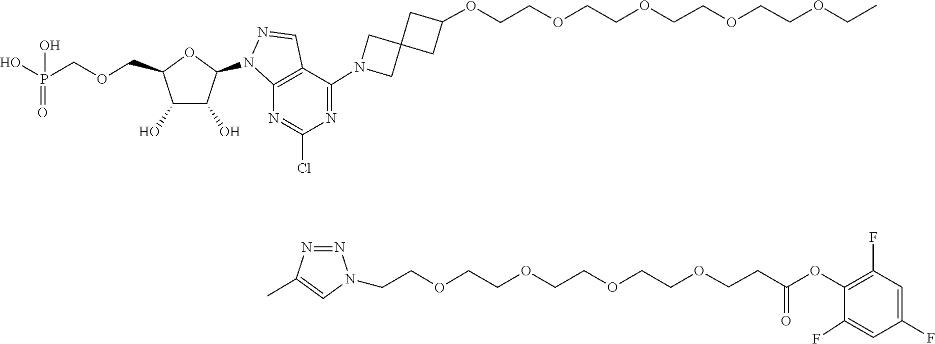 |
| Int-290 | 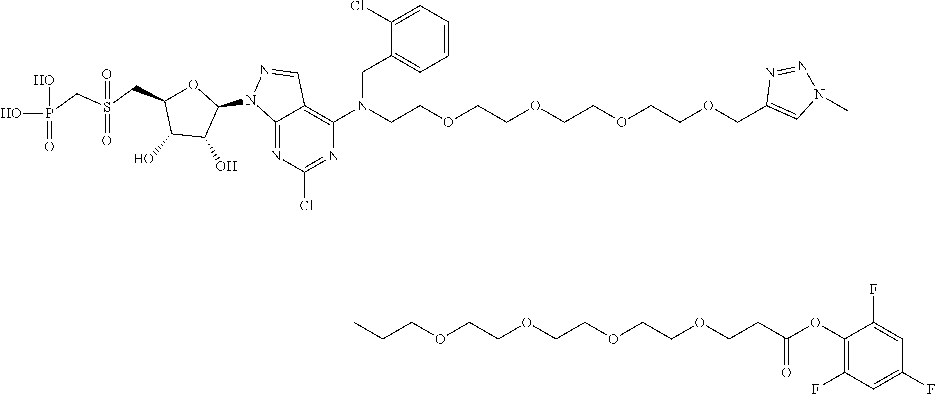 |
| Int-291 | 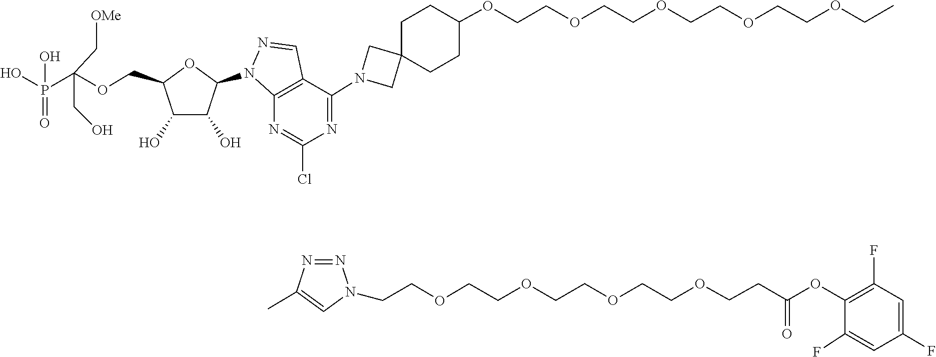 |
| Int-292 | 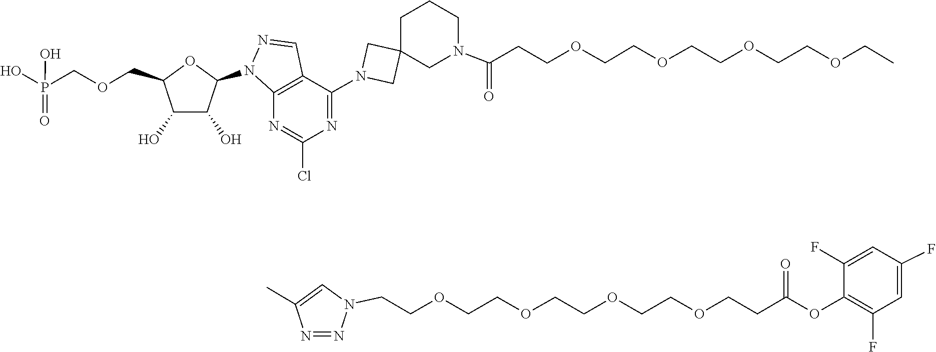 |
| Int-293 | 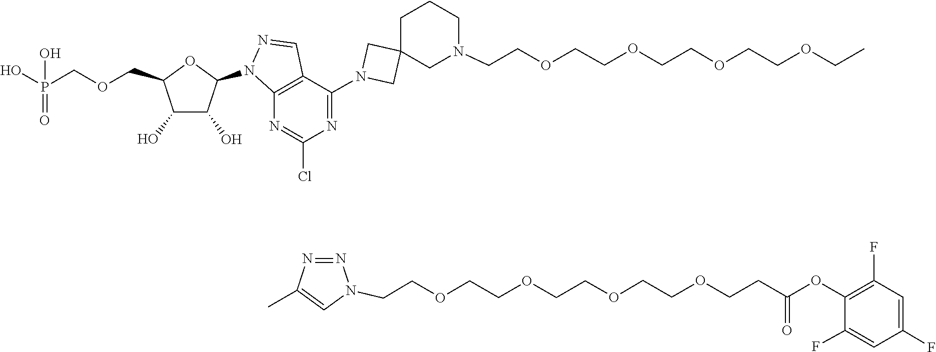 |
| Int-294 | 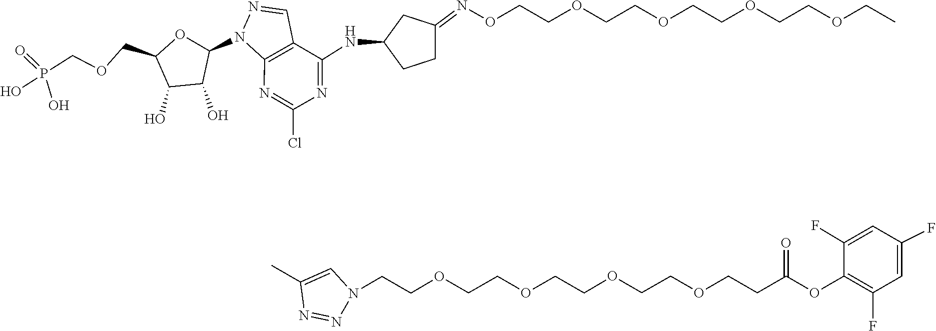 |
| Int-295 | 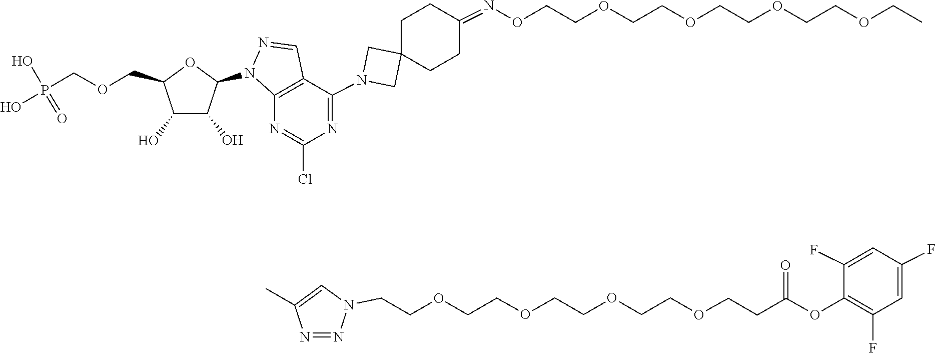 |
| Int-296 | 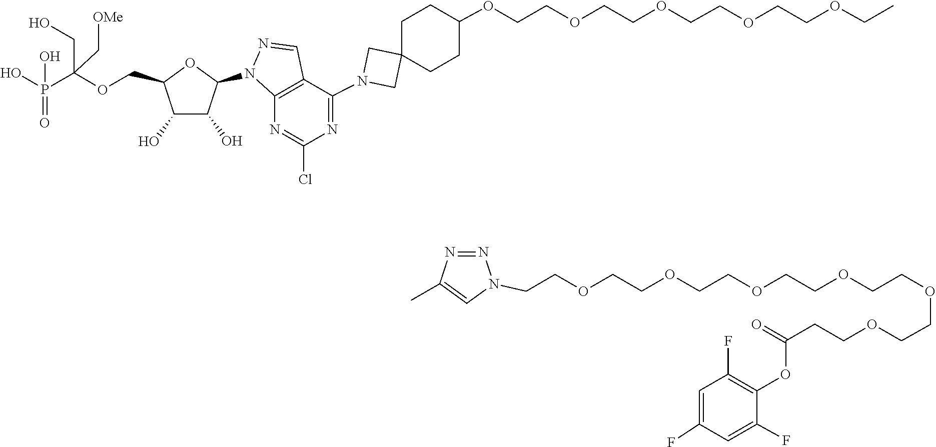 |
| Int-297 | 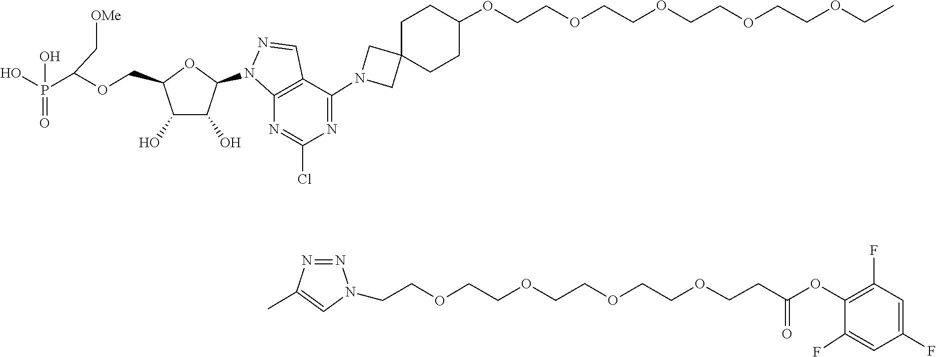 |
| Int-298 | 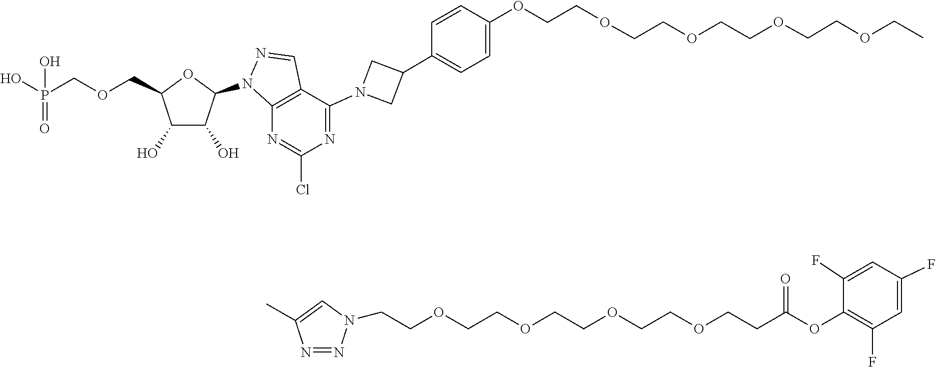 |
| Int-299 | 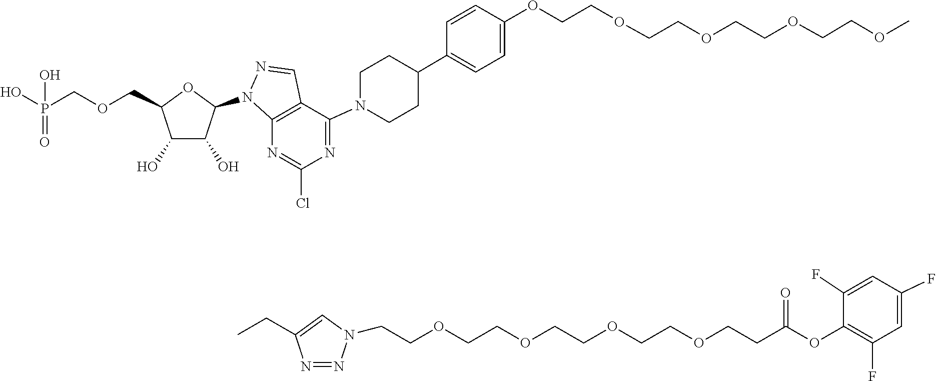 |
| Int-300 | 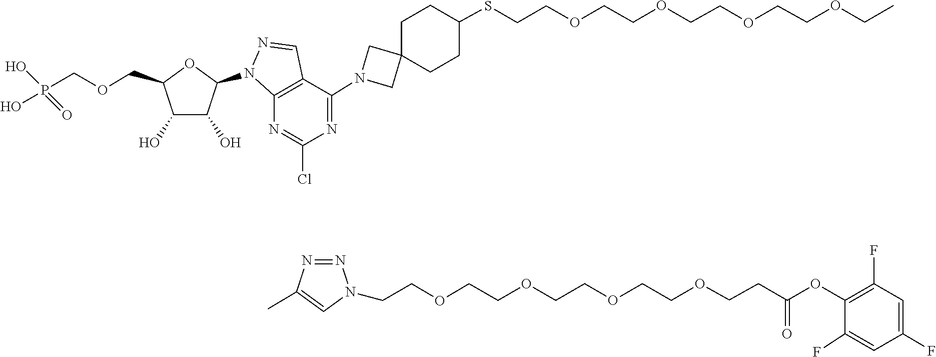 |
| Int-301 | 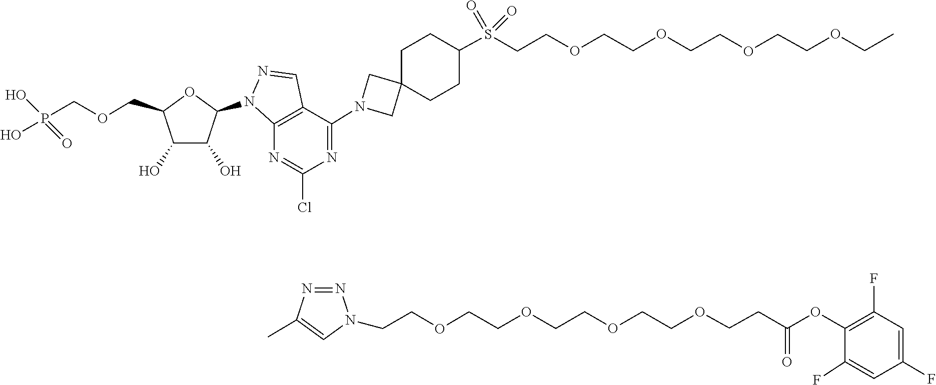 |
| Int-302 | 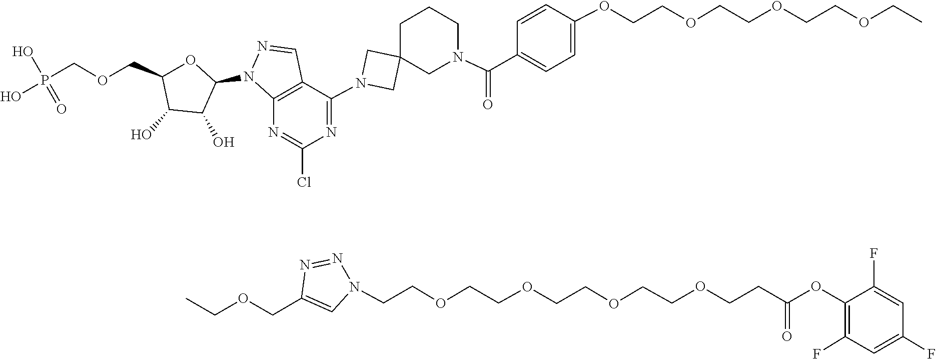 |
| Int-303 | 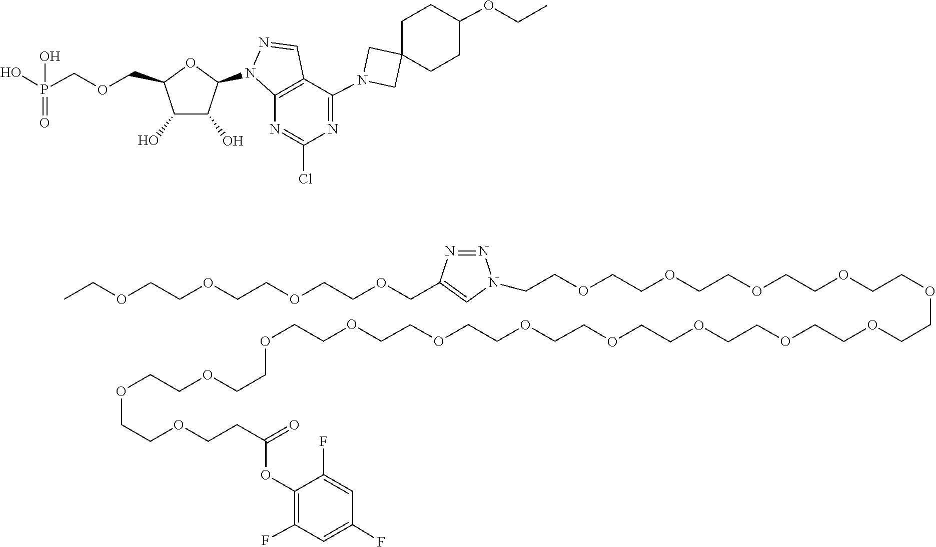 [SMILES: CCOC1CCC2(CC1)CN(c1nc(Cl)nc3c1cnn3[C@@H]1O[C@H](COCP(=O)(O)O)[C@@H](O)[C@H]1O)C2 ; CCOCCOCCOCCOCc1cn(CCOCCOCCOCCOCCOCCOCCOCCOCCOCCOCCOCCOCCOCCOCCOCCOCCC(=O)Oc2c(F)cc(F)cc2F)nn1] |
| Int-304 | 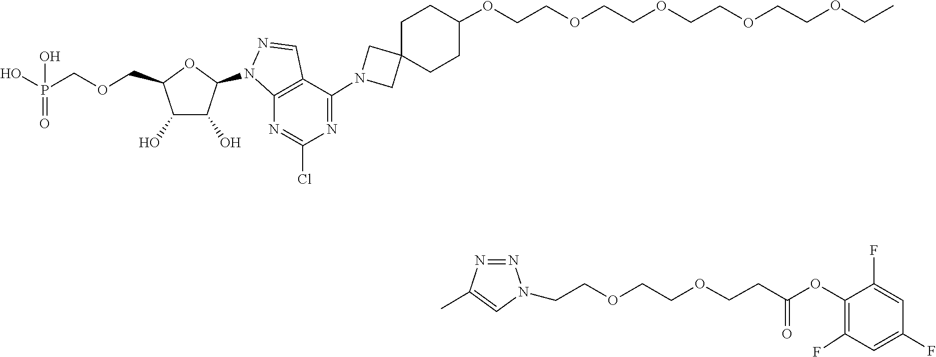 |
| Int-305 | 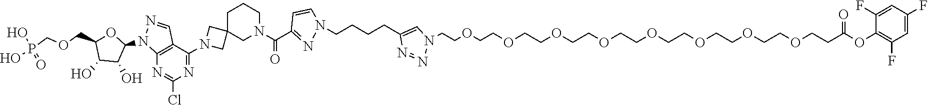 [SMILES: O=C(CCOCCOCCOCCOCCOCCOCCOCCOCCn1cc(CCCCn2ccc(C(=O)N3CCCC4(C3)CN(c3nc(Cl)nc5c3cnn5[C@@H]3O[C@H](COCP(=O)(O)O)[C@@H](O)[C@H]3O)C4)n2)nn1)Oc1c(F)cc(F)cc1F] |
| Int-306 | 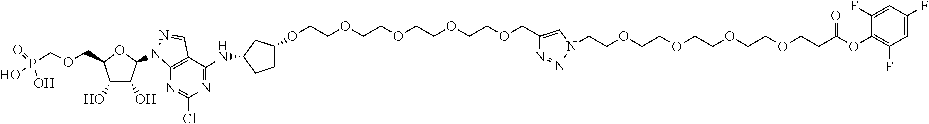 [SMILES: O=C(CCOCCOCCOCCOCCn1cc(COCCOCCOCCOCCO[C@@H]2CC[C@H](Nc3nc(Cl)nc4c3cnn4[C@@H]3O[C@H](COCP(=O)(O)O)[C@@H](O)[C@H]3O)C2)nn1)Oc1c(F)cc(F)cc1F] |
| Int-307 | 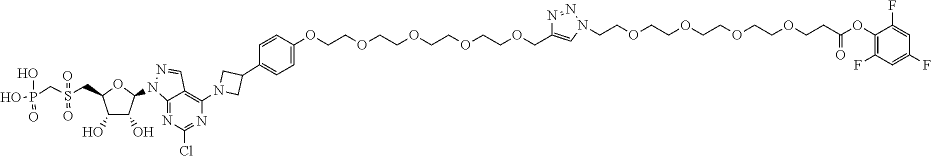 [SMILES: O=C(CCOCCOCCOCCOCCn1cc(COCCOCCOCCOCCOc2ccc(C3CN(c4nc(Cl)nc5c4cnn5[C@@H]4O[C@H](CS(=O)(=O)CP(=O)(O)O)[C@@H](O)[C@H]4O)C3)cc2)nn1)Oc1c(F)cc(F)cc1F] |
| Int-308 | 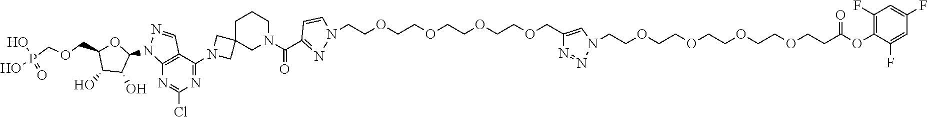 [SMILES: O=C(CCOCCOCCOCCOCCn1cc(COCCOCCOCCOCCn2ccc(C(=O)N3CCCC4(C3)CN(c3nc(Cl)nc5c3cnn5[C@@H]3O[C@H](COCP(=O)(O)O)[C@@H](O)[C@H]3O)C4)n2)nn1)Oc1c(F)cc(F)cc1F] |
| Int-309 | 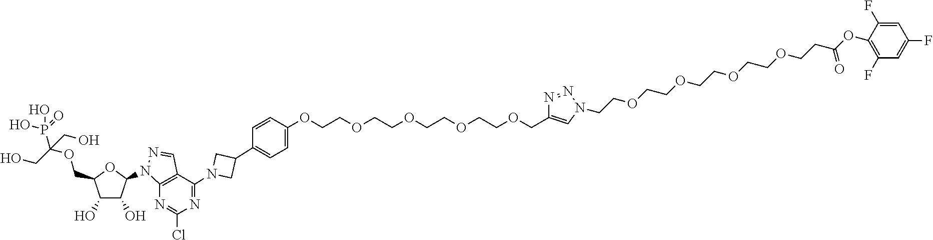 [SMILES: O=C(CCOCCOCCOCCOCCn1cc(COCCOCCOCCOCCOc2ccc(C3CN(c4nc(Cl)nc5c4cnn5[C@@H]4O[C@H](COC(CO)(CO)P(=O)(O)O)[C@@H](O)[C@H]4O)C3)cc2)nn1)Oc1c(F)cc(F)cc1F] |
| Int-310 | 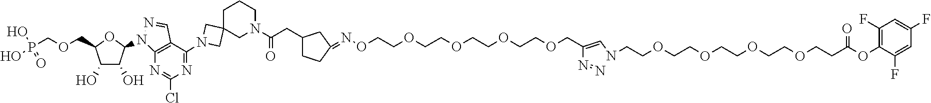 [SMILES: O=C(CCOCCOCCOCCOCCn1cc(COCCOCCOCCOCCO/N=C2\CCC(CC(=O)N3CCCC4(C3)CN(c3nc(Cl)nc5c3cnn5[C@@H]3O[C@H](COCP(=O)(O)O)[C@@H](O)[C@H]3O)C4)C2)nn1)Oc1c(F)cc(F)cc1F] |
| Int-311 | 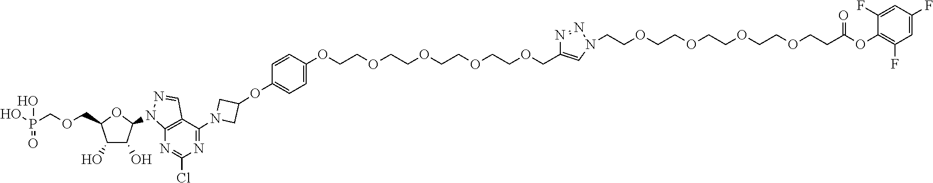 [SMILES: O=C(CCOCCOCCOCCOCCn1cc(COCCOCCOCCOCCOc2ccc(OC3CN(c4nc(Cl)nc5c4cnn5[C@@H]4O[C@H](COCP(=O)(O)O)[C@@H](O)[C@H]4O)C3)cc2)nn1)Oc1c(F)cc(F)cc1F] |
| Int-312 | 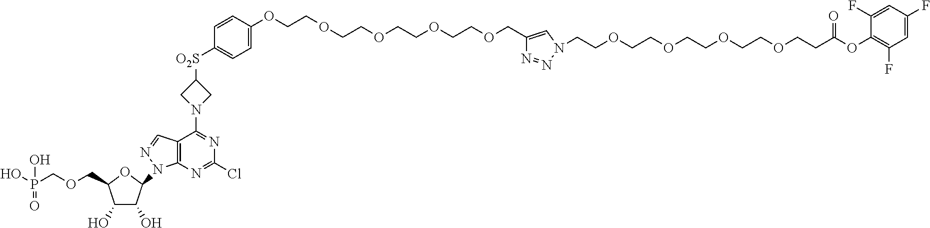 [SMILES: O=C(CCOCCOCCOCCOCCn1cc(COCCOCCOCCOCCOc2ccc(S(=O)(=O)C3CN(c4nc(Cl)nc5c4cnn5[C@@H]4O[C@H](COCP(=O)(O)O)[C@@H](O)[C@H]4O)C3)cc2)nn1)Oc1c(F)cc(F)cc1F] |
| Int-313 | 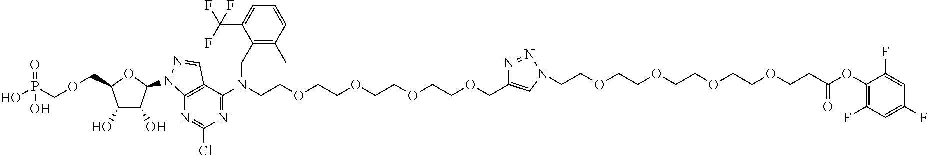 [SMILES: Cc1cccc(C(F)(F)F)c1CN(CCOCCOCCOCCOCc1cn(CCOCCOCCOCCOCCC(=O)Oc2c(F)cc(F)cc2F)nn1)c1nc(Cl)nc2c1cnn2[C@@H]1O[C@H](COCP(=O)(O)O)[C@@H](O)[C@H]1O] |
| Int-314 | 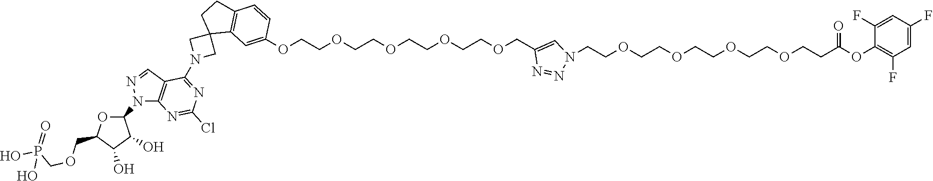 [SMILES: O=C(CCOCCOCCOCCOCCn1cc(COCCOCCOCCOCCOc2ccc3c(c2)C2(CC3)CN(c3nc(Cl)nc4c3cnn4[C@@H]3O[C@H](COCP(=O)(O)O)[C@@H](O)[C@H]3O)C2)nn1)Oc1c(F)cc(F)cc1F] |
| Int-315 | 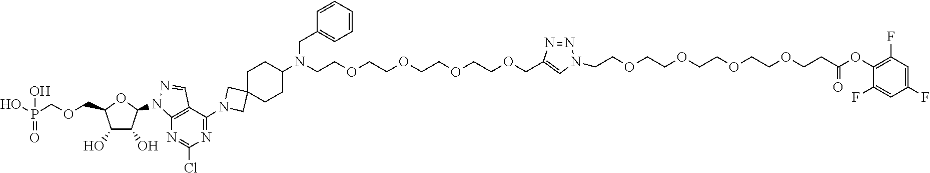 [SMILES: O=C(CCOCCOCCOCCOCCn1cc(COCCOCCOCCOCCN(Cc2ccccc2)C2CCC3(CC2)CN(c2nc(Cl)nc4c2cnn4[C@@H]2O[C@H](COCP(=O)(O)O)[C@@H](O)[C@H]2O)C3)nn1)Oc1c(F)cc(F)cc1F] |
| Int-316 | 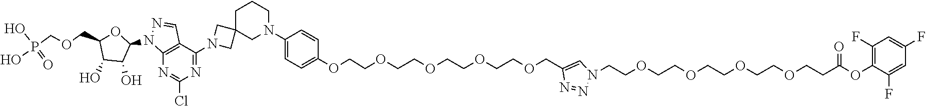 [SMILES: O=C(CCOCCOCCOCCOCCn1cc(COCCOCCOCCOCCOc2ccc(N3CCCC4(C3)CN(c3nc(Cl)nc5c3cnn5[C@@H]3O[C@H](COCP(=O)(O)O)[C@@H](O)[C@H]3O)C4)cc2)nn1)Oc1c(F)cc(F)cc1F] |
| Int-317 | 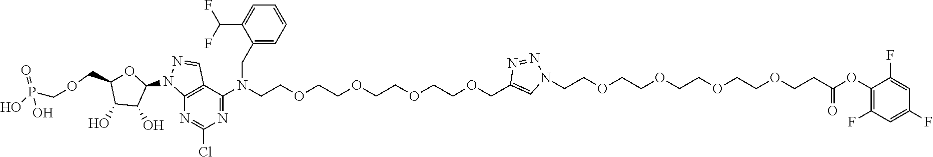 [SMILES: O=C(CCOCCOCCOCCOCCn1cc(COCCOCCOCCOCCN(Cc2ccccc2C(F)F)c2nc(Cl)nc3c2cnn3[C@@H]2O[C@H](COCP(=O)(O)O)[C@@H](O)[C@H]2O)nn1)Oc1c(F)cc(F)cc1F] |
| Int-318 | 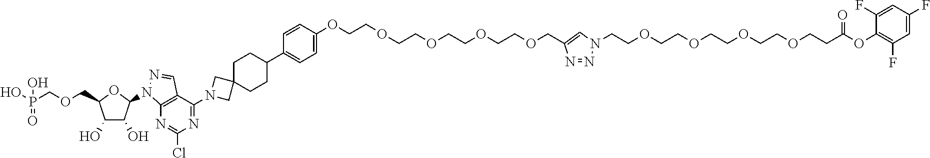 [SMILES: O=C(CCOCCOCCOCCOCCn1cc(COCCOCCOCCOCCOc2ccc(C3CCC4(CC3)CN(c3nc(Cl)nc5c3cnn5[C@@H]3O[C@H](COCP(=O)(O)O)[C@@H](O)[C@H]3O)C4)cc2)nn1)Oc1c(F)cc(F)cc1F] |
| Int-320 | 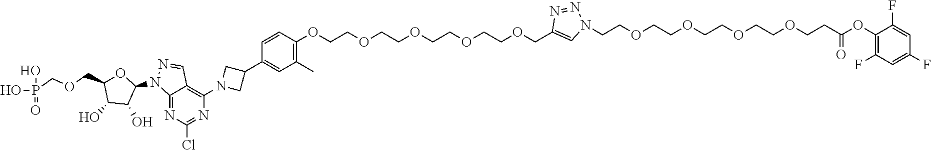 [SMILES: Cc1cc(C2CN(c3nc(Cl)nc4c3cnn4[C@@H]3O[C@H](COCP(=O)(O)O)[C@@H](O)[C@H]3O)C2)ccc1OCCOCCOCCOCCOCc1cn(CCOCCOCCOCCOCCC(=O)Oc2c(F)cc(F)cc2F)nn1] |
| Int-321 | 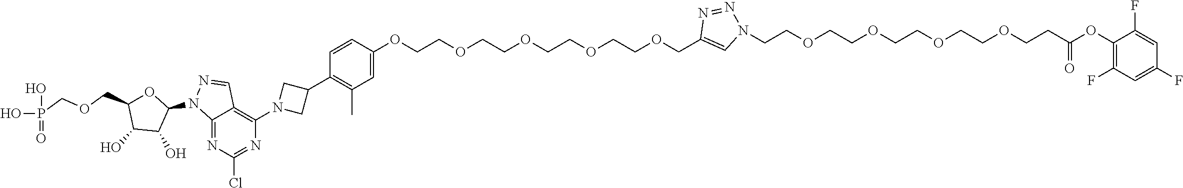 [SMILES: Cc1cc(OCCOCCOCCOCCOCc2cn(CCOCCOCCOCCOCCC(=O)Oc3c(F)cc(F)cc3F)nn2)ccc1C1CN(c2nc(Cl)nc3c2cnn3[C@@H]2O[C@H](COCP(=O)(O)O)[C@@H](O)[C@H]2O)C1] |
| Int-322 | 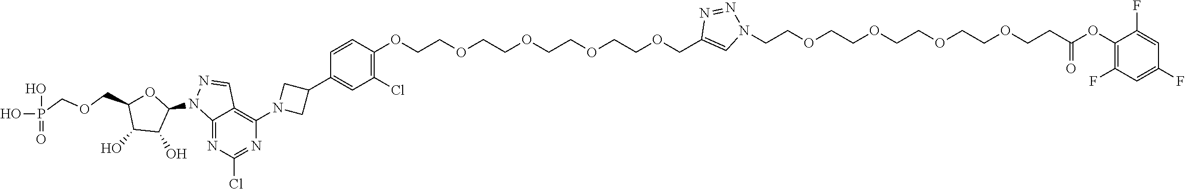 [SMILES: O=C(CCOCCOCCOCCOCCn1cc(COCCOCCOCCOCCOc2ccc(C3CN(c4nc(Cl)nc5c4cnn5[C@@H]4O[C@H](COCP(=O)(O)O)[C@@H](O)[C@H]4O)C3)cc2Cl)nn1)Oc1c(F)cc(F)cc1F] |
| Int-323 | 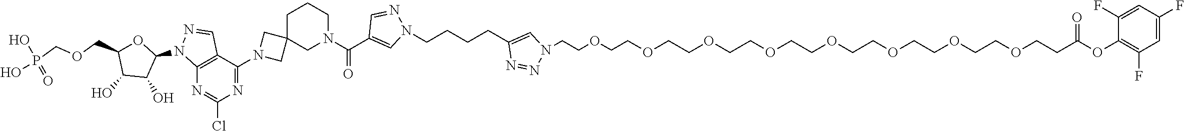 [SMILES: O=C(CCOCCOCCOCCOCCOCCOCCOCCOCCn1cc(CCCCn2cc(C(=O)N3CCCC4(C3)CN(c3nc(Cl)nc5c3cnn5[C@@H]3O[C@H](COCP(=O)(O)O)[C@@H](O)[C@H]3O)C4)cn2)nn1)Oc1c(F)cc(F)cc1F] |
| Int-324 | 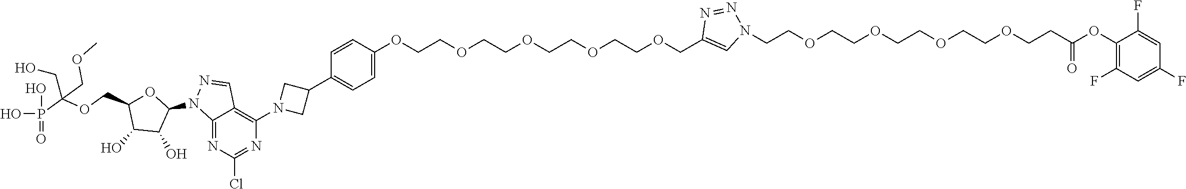 [SMILES: COCC(CO)(OC[C@H]1O[C@@H](n2ncc3c(N4CC(c5ccc(OCCOCCOCCOCCOCc6cn(CCOCCOCCOCCOCCC(=O)Oc7c(F)cc(F)cc7F)nn6)cc5)C4)nc(Cl)nc32)[C@H](O)[C@@H]1O)P(=O)(O)O] |
| Int-325 | 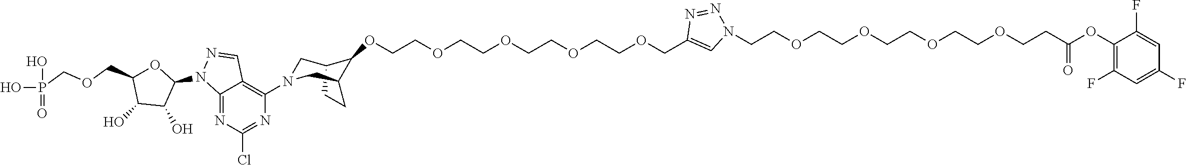 [SMILES: O=C(CCOCCOCCOCCOCCn1cc(COCCOCCOCCOCCO[C@@H]2[C@@H]3CC[C@H]2CN(c2nc(Cl)nc4c2cnn4[C@@H]2O[C@H](COCP(=O)(O)O)[C@@H](O)[C@H]2O)C3)nn1)Oc1c(F)cc(F)cc1F] |
| Int-326 | 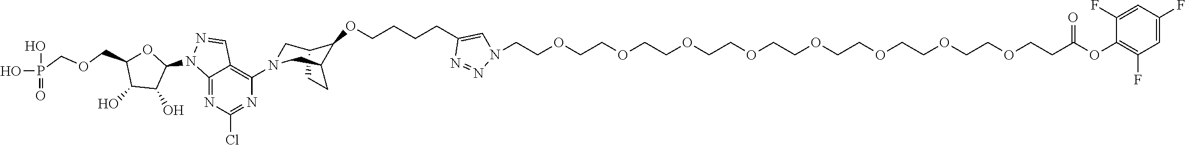 [SMILES: O=C(CCOCCOCCOCCOCCOCCOCCOCCOCCn1cc(CCCCO[C@@H]2[C@@H]3CC[C@H]2CN(c2nc(Cl)nc4c2cnn4[C@@H]2O[C@H](COCP(=O)(O)O)[C@@H](O)[C@H]2O)C3)nn1)Oc1c(F)cc(F)cc1F] |
| Int-327 | 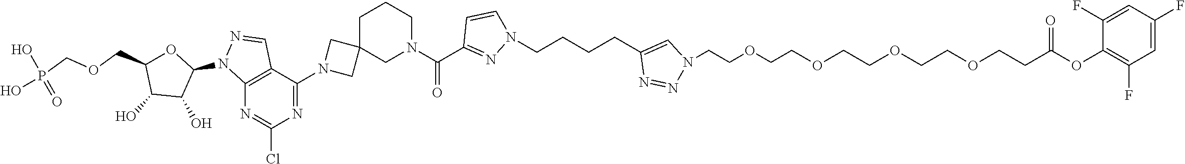 [SMILES: O=C(CCOCCOCCOCCOCCn1cc(CCCCn2ccc(C(=O)N3CCCC4(C3)CN(c3nc(Cl)nc5c3cnn5[C@@H]3O[C@H](COCP(=O)(O)O)[C@@H](O)[C@H]3O)C4)n2)nn1)Oc1c(F)cc(F)cc1F] |
| Int-328 | 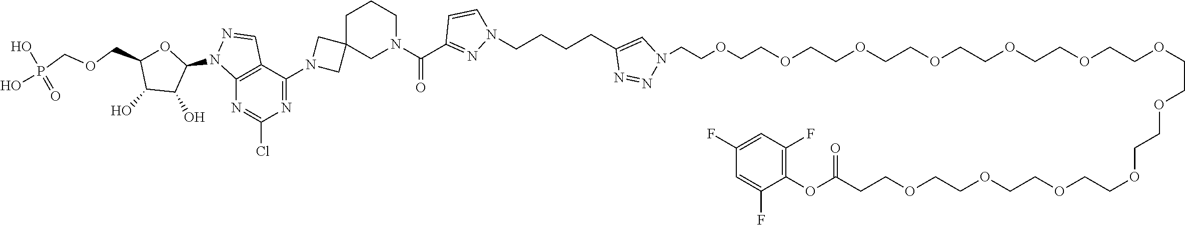 [SMILES: O=C(CCOCCOCCOCCOCCOCCOCCOCCOCCOCCOCCOCCOCCn1cc(CCCCn2ccc(C(=O)N3CCCC4(C3)CN(c3nc(Cl)nc5c3cnn5[C@@H]3O[C@H](COCP(=O)(O)O)[C@@H](O)[C@H]3O)C4)n2)nn1)Oc1c(F)cc(F)cc1F] |
| Int-329 | 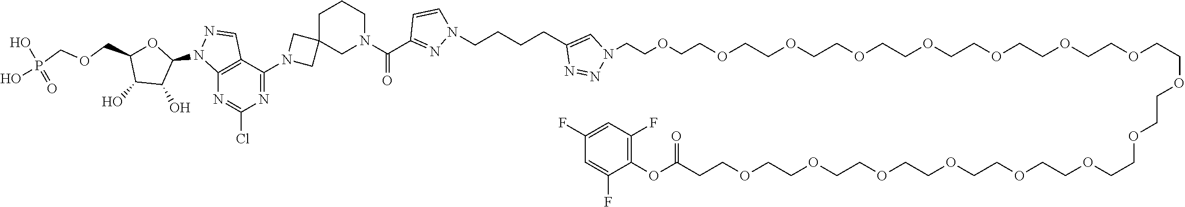 [SMILES: O=C(CCOCCOCCOCCOCCOCCOCCOCCOCCOCCOCCOCCOCCOCCOCCOCCOCCn1cc(CCCCn2ccc(C(=O)N3CCCC4(C3)CN(c3nc(Cl)nc5c3cnn5[C@@H]3O[C@H](COCP(=O)(O)O)[C@@H](O)[C@H]3O)C4)n2)nn1)Oc1c(F)cc(F)cc1F] |
| Int-330 | 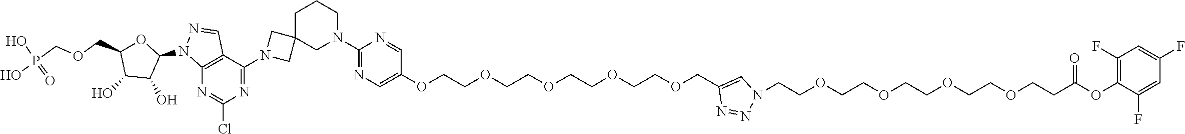 [SMILES: O=C(CCOCCOCCOCCOCCn1cc(COCCOCCOCCOCCOc2cnc(N3CCCC4(C3)CN(c3nc(Cl)nc5c3cnn5[C@@H]3O[C@H](COCP(=O)(O)O)[C@@H](O)[C@H]3O)C4)nc2)nn1)Oc1c(F)cc(F)cc1F] |
| Int-331 | 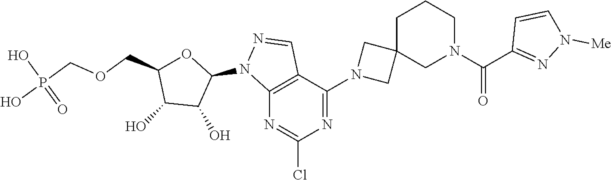 [SMILES: Cn1ccc(C(=O)N2CCCC3(C2)CN(c2nc(Cl)nc4c2cnn4[C@@H]2O[C@H](COCP(=O)(O)O)[C@@H](O)[C@H]2O)C3)n1] |
| Int-332 | 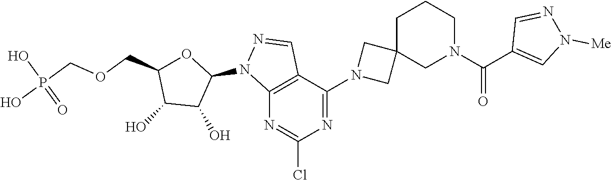 [SMILES: Cn1cc(C(=O)N2CCCC3(C2)CN(c2nc(Cl)nc4c2cnn4[C@@H]2O[C@H](COCP(=O)(O)O)[C@@H](O)[C@H]2O)C3)cn1] |
| Int-333 | 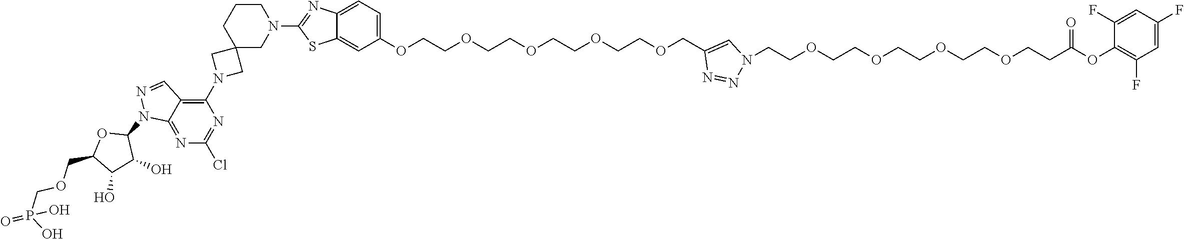 [SMILES: O=C(CCOCCOCCOCCOCCn1cc(COCCOCCOCCOCCOc2ccc3nc(N4CCCC5(C4)CN(c4nc(Cl)nc6c4cnn6[C@@H]4O[C@H](COCP(=O)(O)O)[C@@H](O)[C@H]4O)C5)sc3c2)nn1)Oc1c(F)cc(F)cc1F] |
| Int-334 | 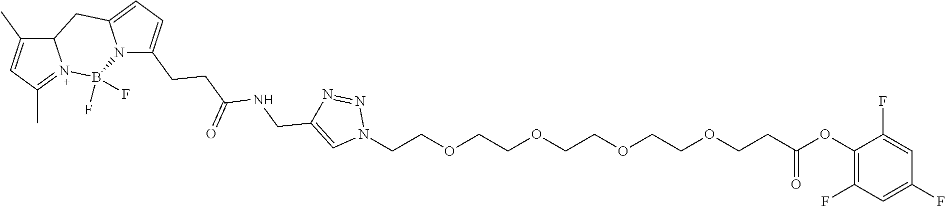 |
| Int-335 | 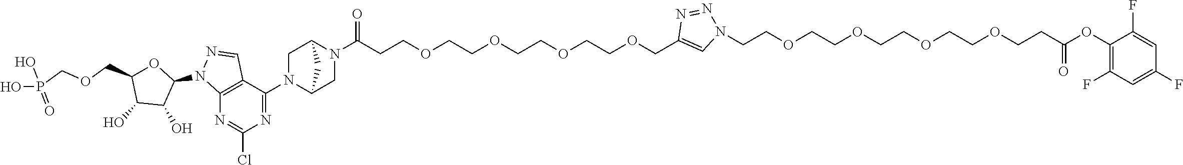 [SMILES: O=C(CCOCCOCCOCCOCCn1cc(COCCOCCOCCOCCC(=O)N2C[C@@H]3C[C@H]2CN3c2nc(Cl)nc3c2cnn3[C@@H]2O[C@H](COCP(=O)(O)O)[C@@H](O)[C@H]2O)nn1)Oc1c(F)cc(F)cc1F] |

The disclosure also features a conjugate of Table 2. Each conjugate of Table 2 corresponds to a conjugate of formula (D-I) or (M-I). Conjugates of Table 2 include conjugates formed by the covalent reaction of an Int of Table 1 with E. Conjugates of table 2 further include conjugates formed by the covalent reaction of an Int of Table 1 with a linker which is in turn conjugated to E. In some embodiments, the reactive moiety of the Int (e.g., the alkyne or azido group) reacts with a corresponding reactive group (e.g., an alkyne or azido group) of a linker covalently attached to E, such that an Int of Table 1 is covalently attached to E. In some embodiments, the reactive moiety of the Int (e.g., the phenyl ester group, e.g., tetrafluorophenyl ester or trifluorophenyl ester group) reacts with a corresponding reactive group (e.g., nitrogen or sulfur atom) of an amino acid side chain of E, such that an Int of Table 1 is covalently attached to E.

In some embodiments in any conjugate of Table 2, n is 1 or 2. When n is 1, E includes an Fc domain monomer (e.g., an Fc domain monomer having the sequence of any one of SEQ ID NOs: 1-112 and 115-120. When n is 2, each E includes an Fc domain monomer (e.g., an Fc domain monomer having the sequence of any one of SEQ ID NOs: 1-112 and 115-120), and the Fc domain monomers dimerize to form and Fc domain.

In some embodiments in any conjugate of Table 2, T is an integer from 1 to 20 (e.g., 1, 2, 3, 4, 5, 6, 7, 8, 9, 10, 11, 12, 13, 14, 15, 16, 17, 18, 19, or 20). The disclosure also provides a population of any of the conjugates of Table 2 wherein the average value of T is 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20.

The squiggly line in the conjugates of Table 2 indicates that each Int is covalently attached to an amino acid side chain in E (e.g., the nitrogen atom of a surface exposed lysine or the sulfur atom of a surface exposed cysteine in E), or a pharmaceutically acceptable salt thereof.

The disclosure also provides a conjugate of Table 2, wherein the conjugate is produced by conjugation (e.g., via a linker) of an Int of Table 1 to an Fc domain or an Fc domain monomer.

TABLE 2
Conjugates corresponding to selected intermediates of Table 1

| Int | Conjugate Structure |
|---|---|
| Int- 251 | 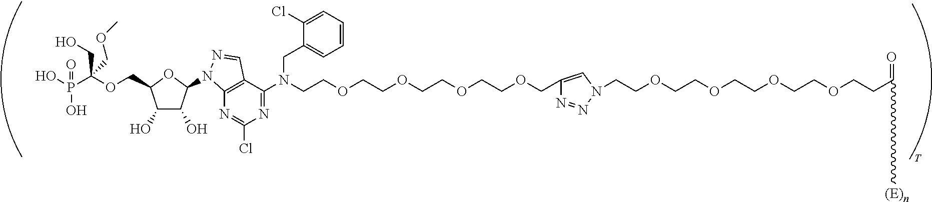 |
| Int- 252 | 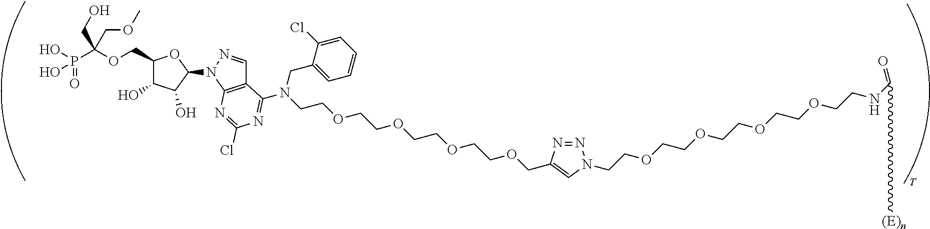 |
| Int- 255 | 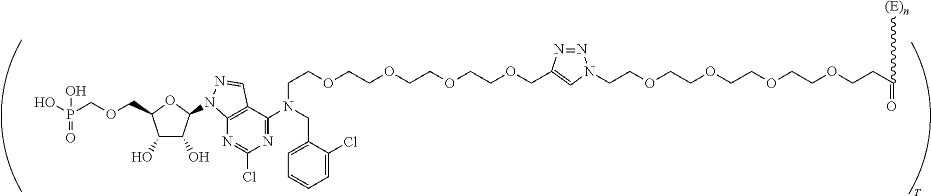 |
| Int- 256 | 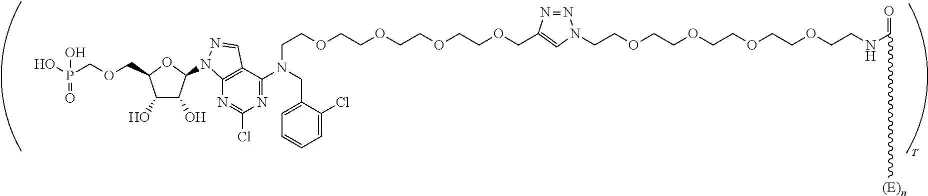 |
| Int- 257 | 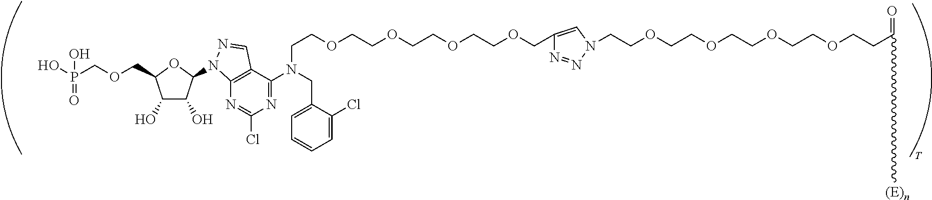 |
| Int- 258 | 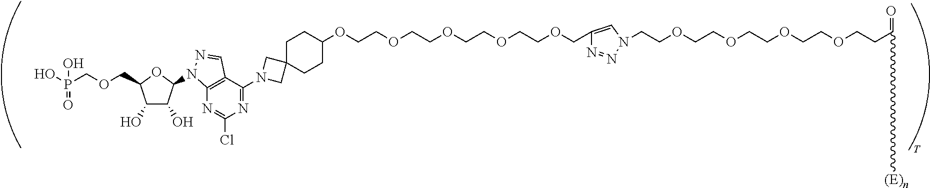 |
| Int- 259 | 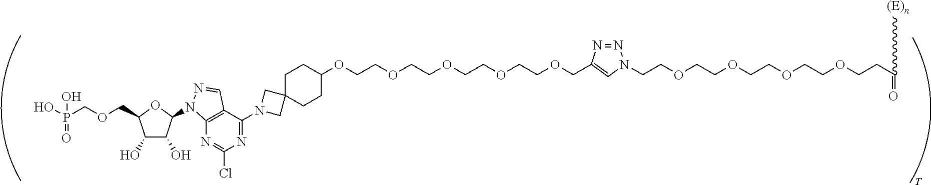 |
| Int- 267 | 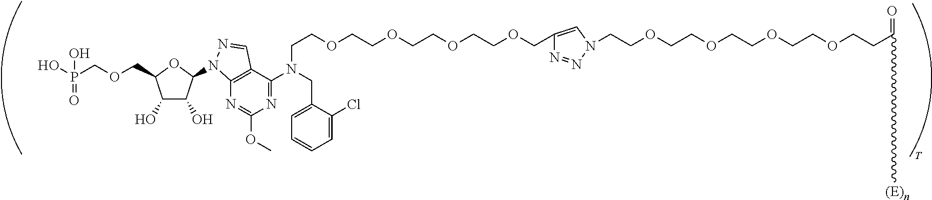 |
| Int- 268 | 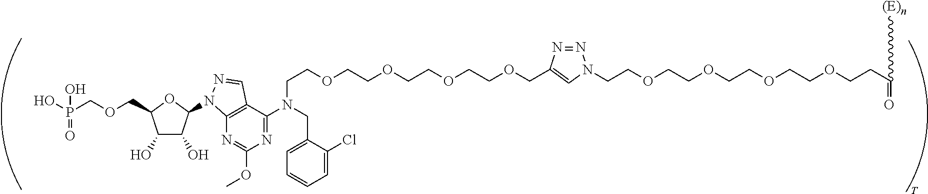 |
| Int- 269 | 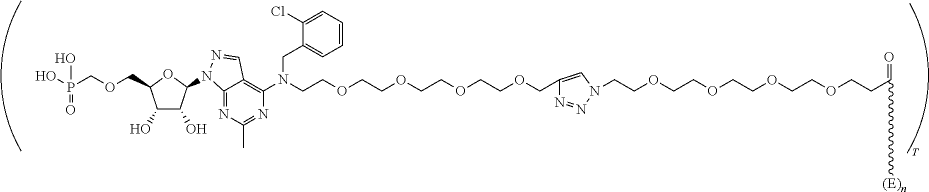 |
| Int- 270 | 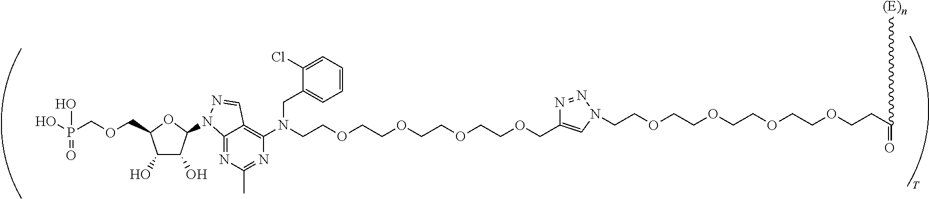 |
| Int- 271 | 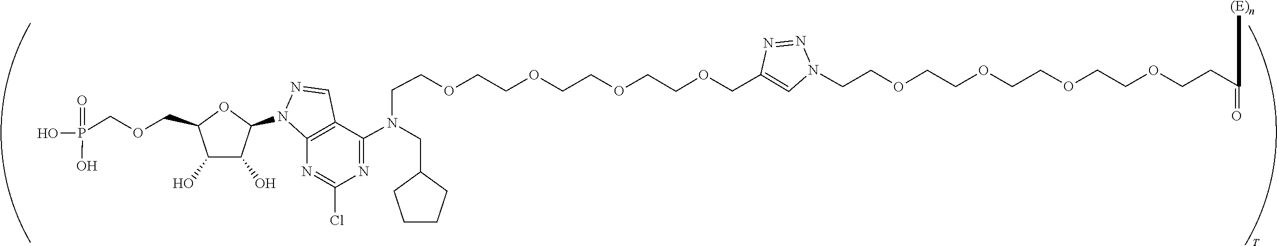 |
| Int- 272 | 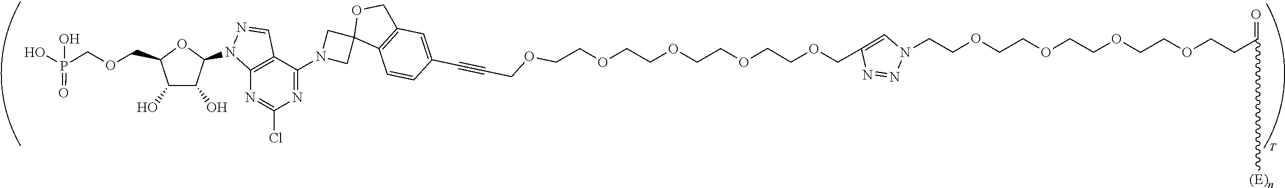 |
| Int- 273 | 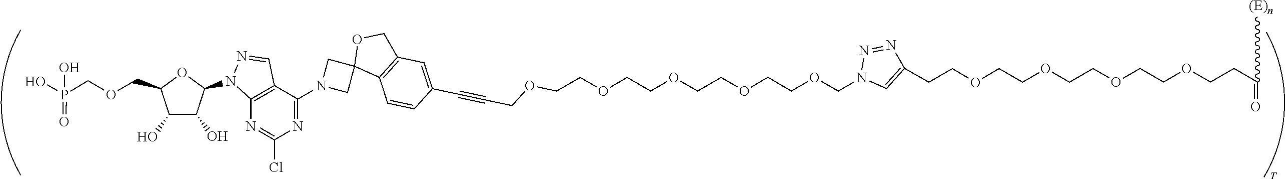 |
| Int- 275 | 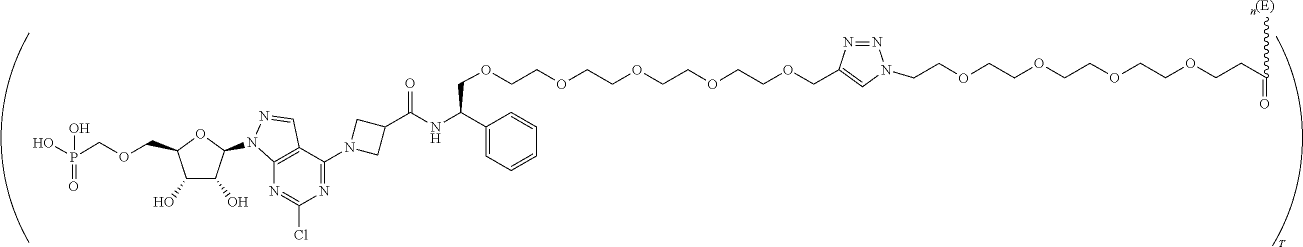 |
| Int- 276 | 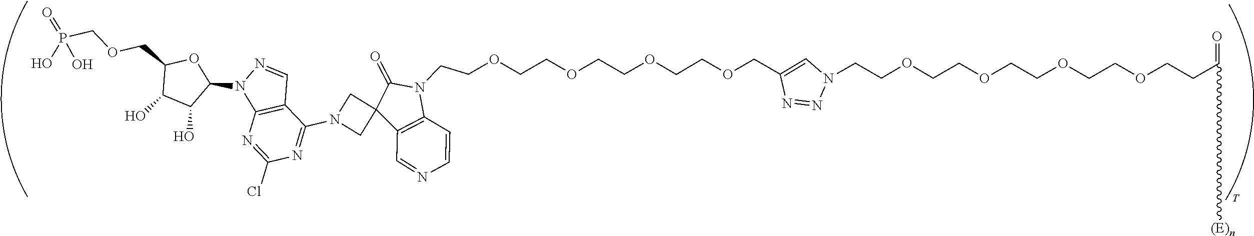 |
| Int- 277 | 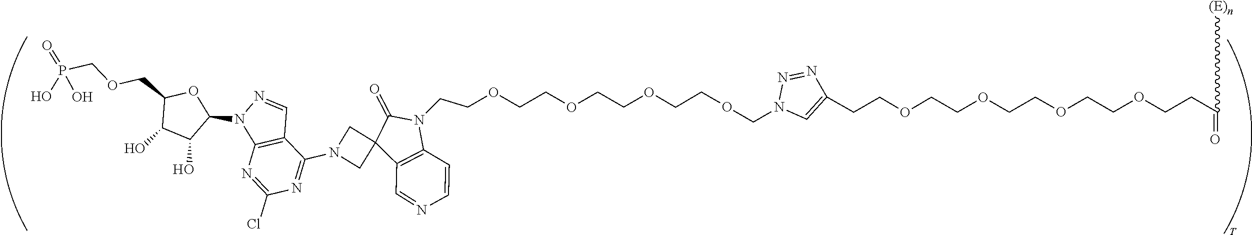 |
| Int- 278 | 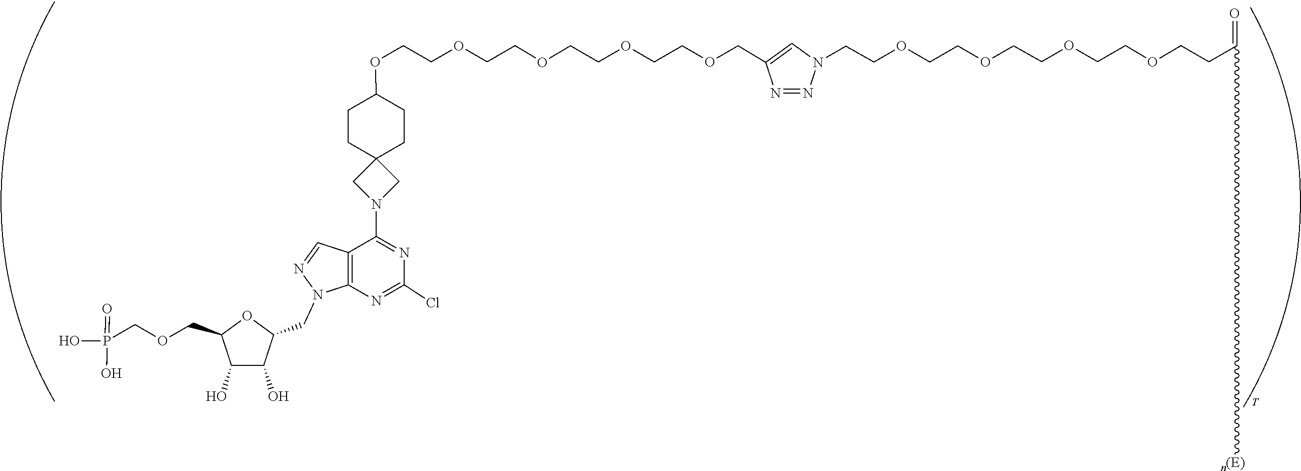 |
| Int- 279 | 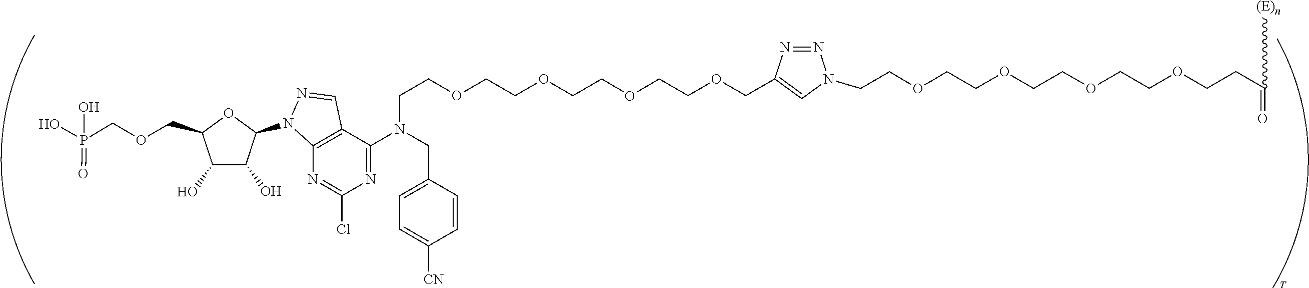 |
| Int- 280 | 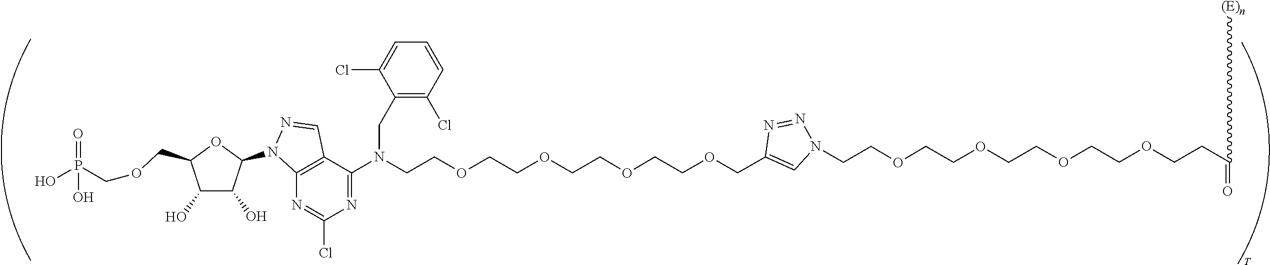 |
| Int- 281 | 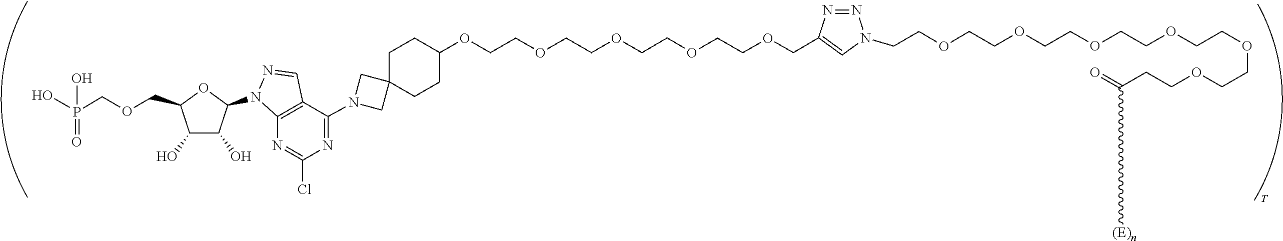 |
| Int- 282 | 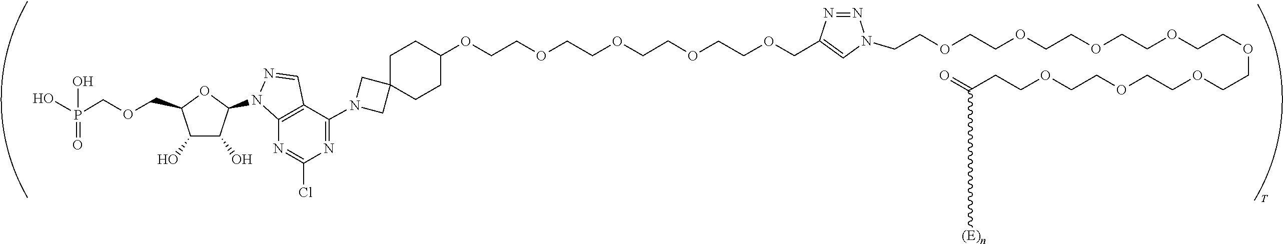 |
| Int- 283 | 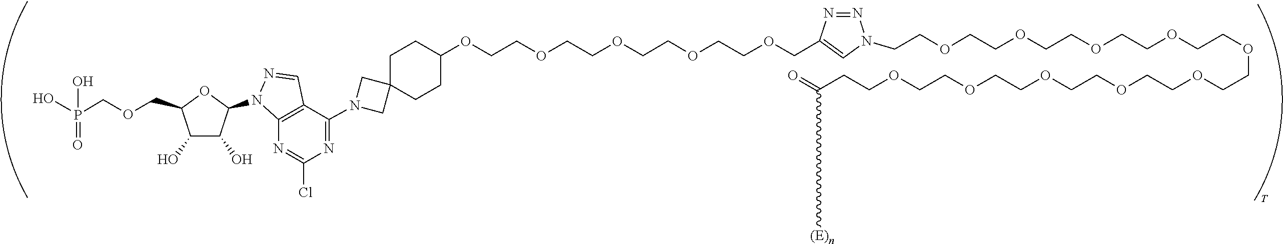 |
| Int- 284 | 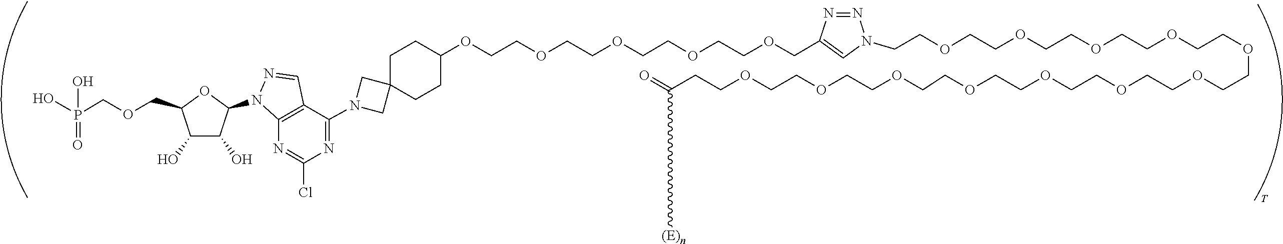 |
| Int- 285 | 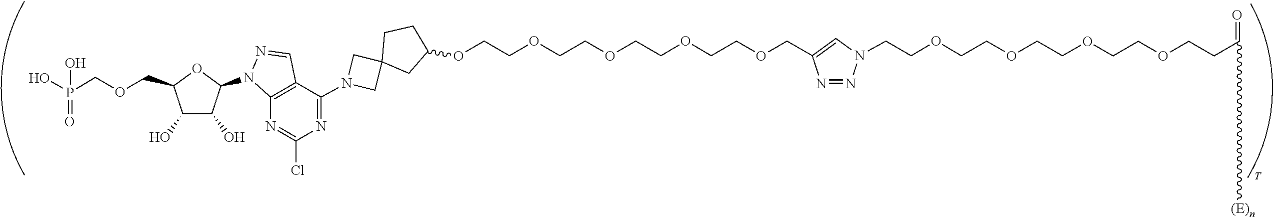 |
| Int- 286 | 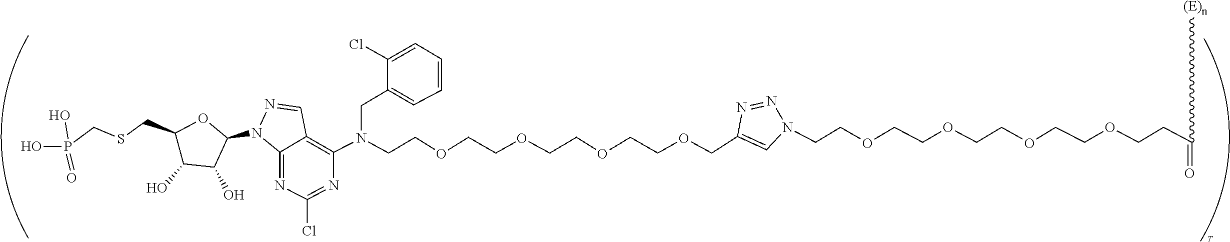 |
| Int- 288 | 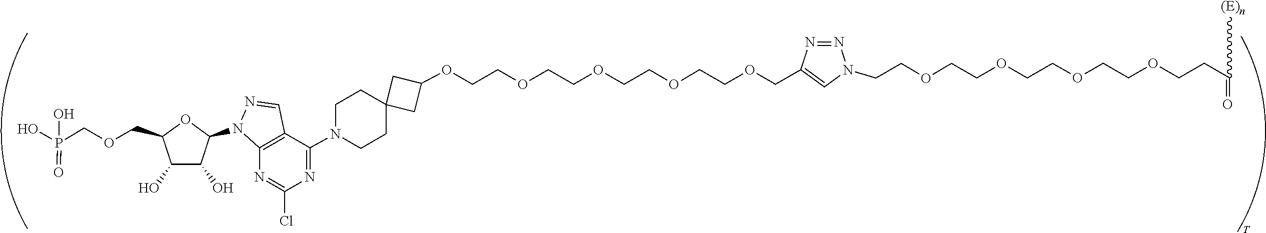 |
| Int- 289 | 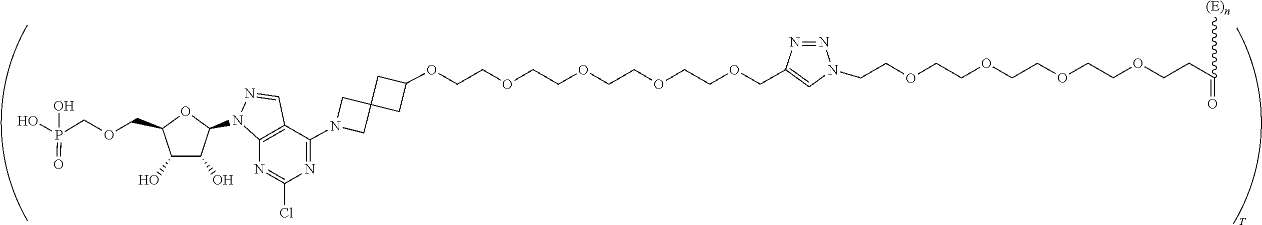 |
| Int- 290 | 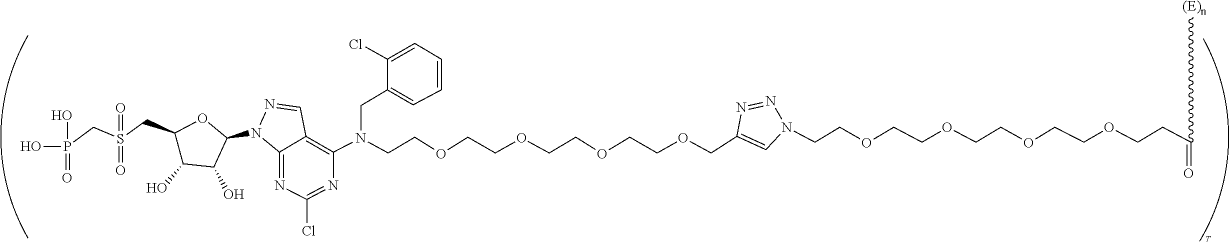 |
| Int- 291 | 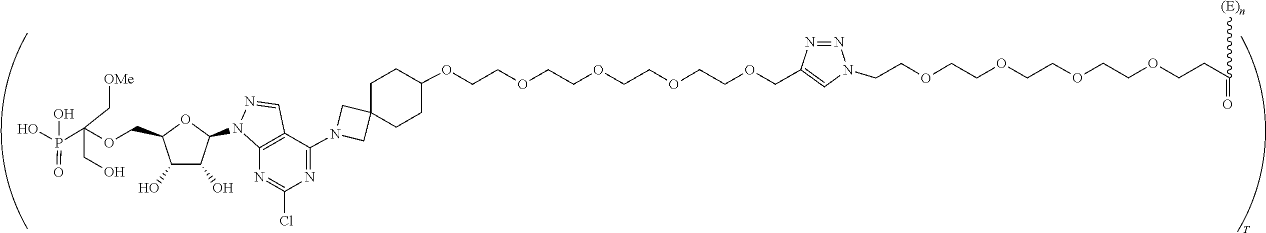 |
| Int- 292 | 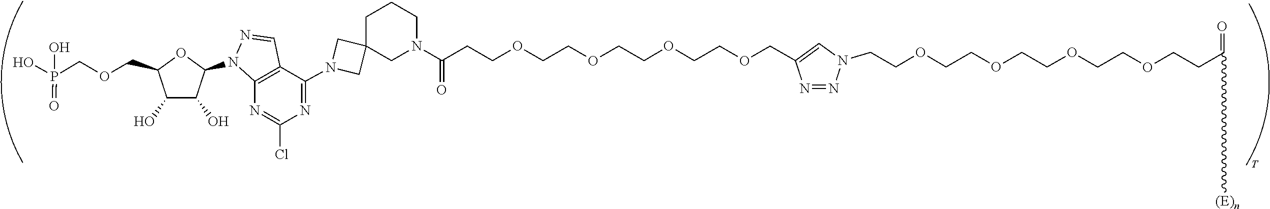 |
| Int- 293 | 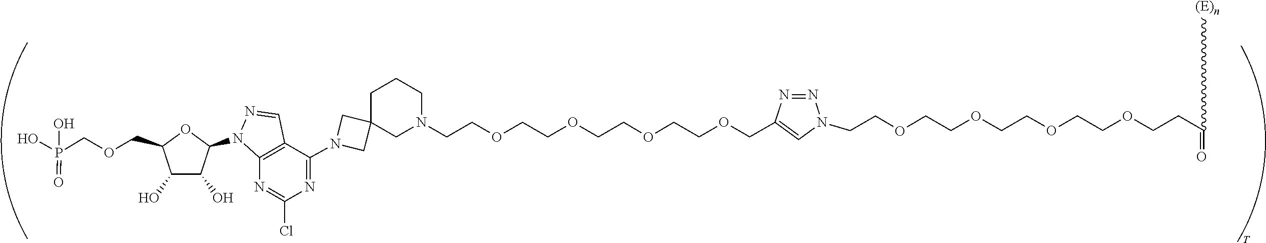 |
| Int- 294 | 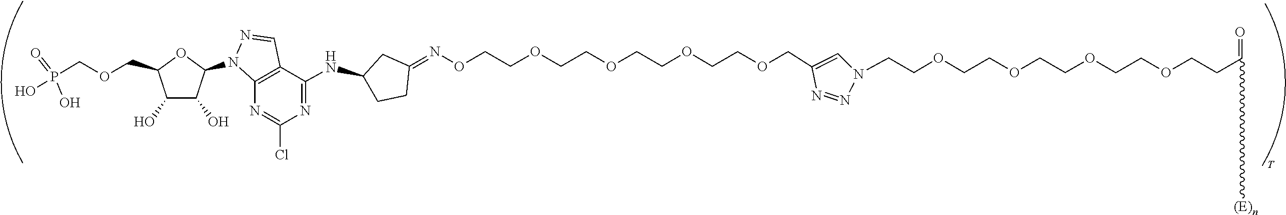 |
| Int- 295 | 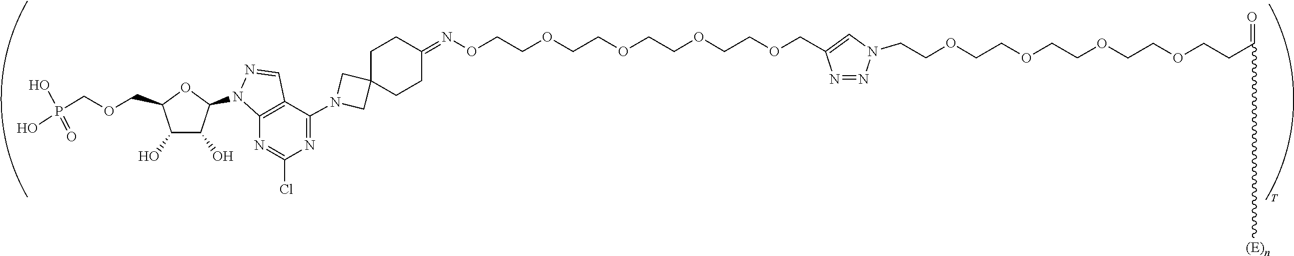 |
| Int- 296 | 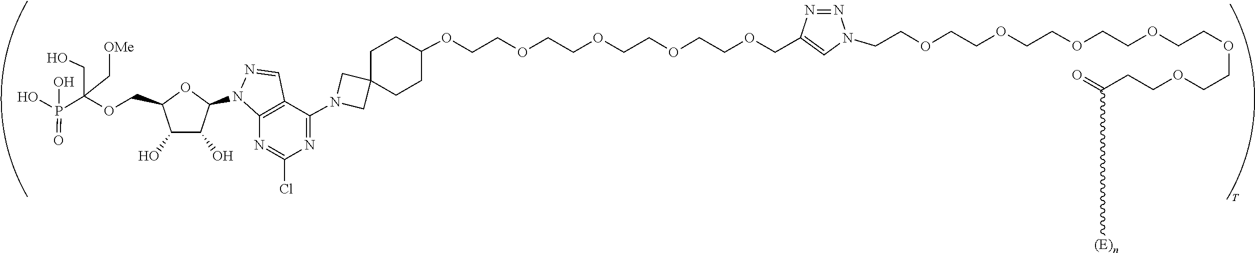 |
| Int- 297 | 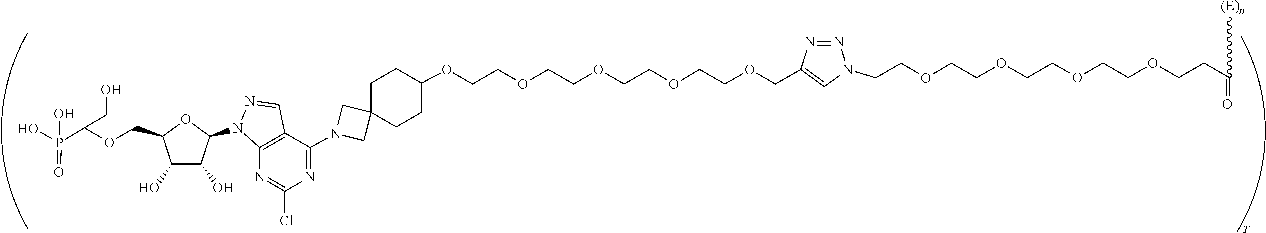 |
| Int- 298 | 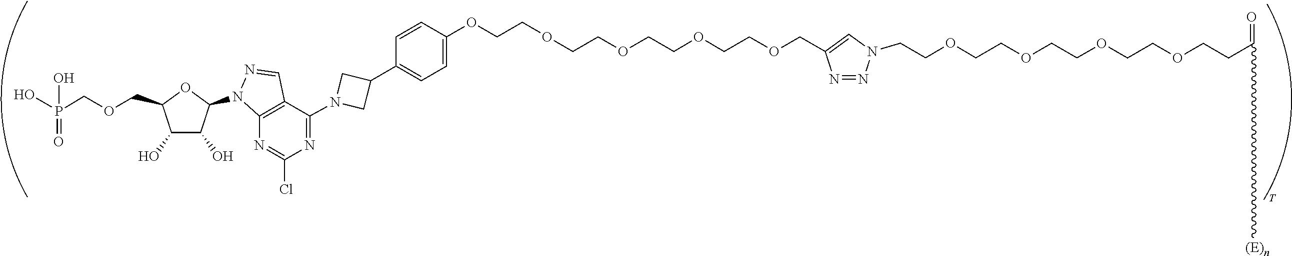 |
| Int- 299 | 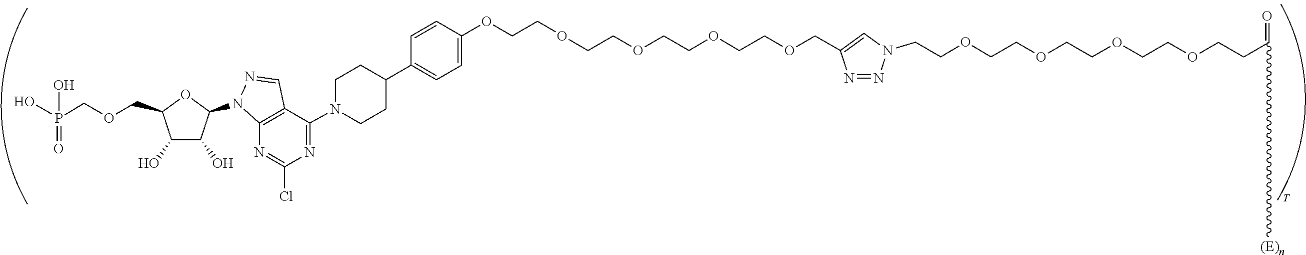 |
| Int- 300 | 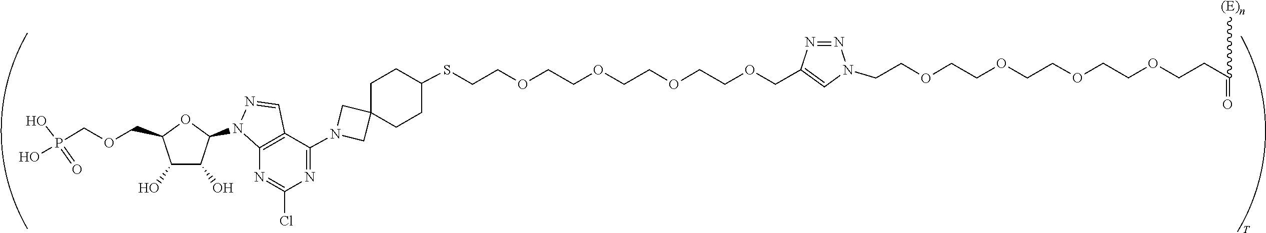 |
| Int- 301 | 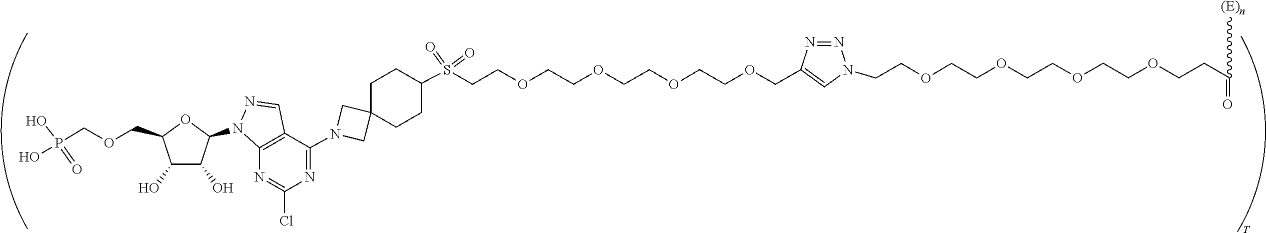 |
| Int- 302 | 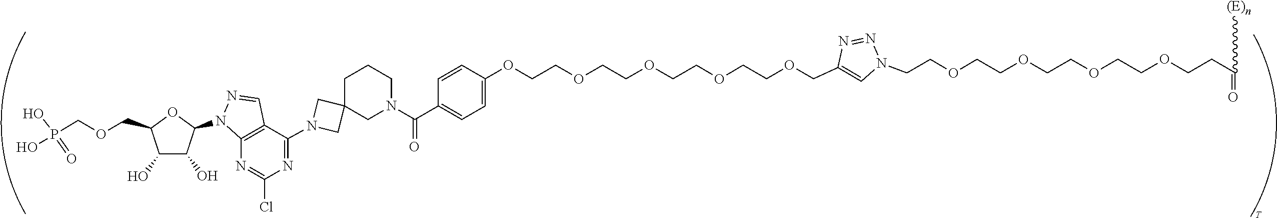 |
| Int- 303 | 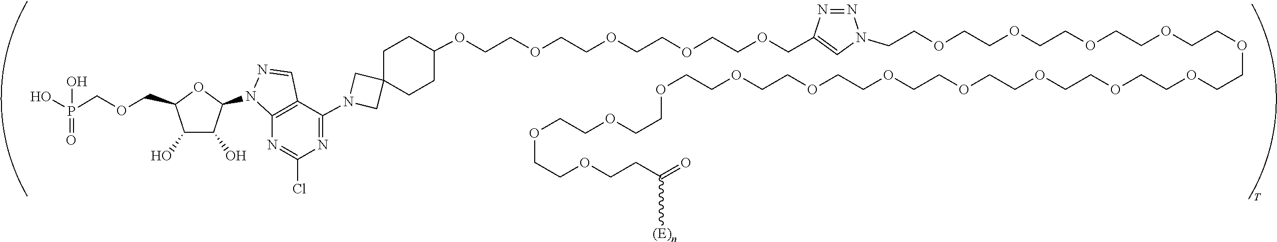 |
| Int- 304 | 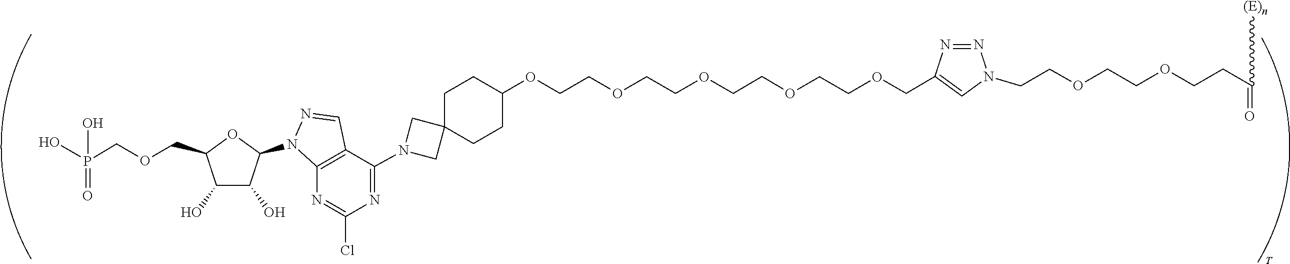 |
| Int- 305 | 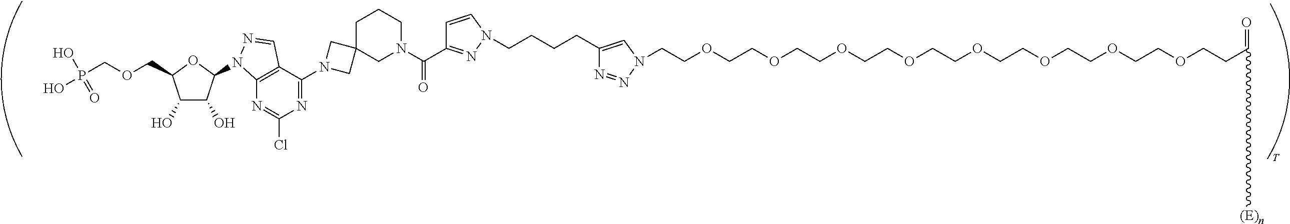 |
| Int- 306 | 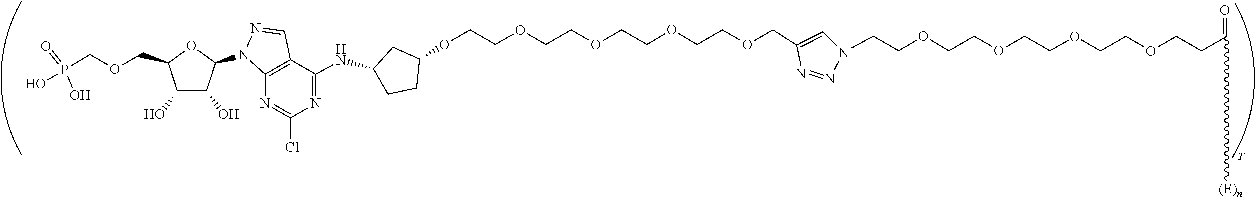 |
| Int- 307 | 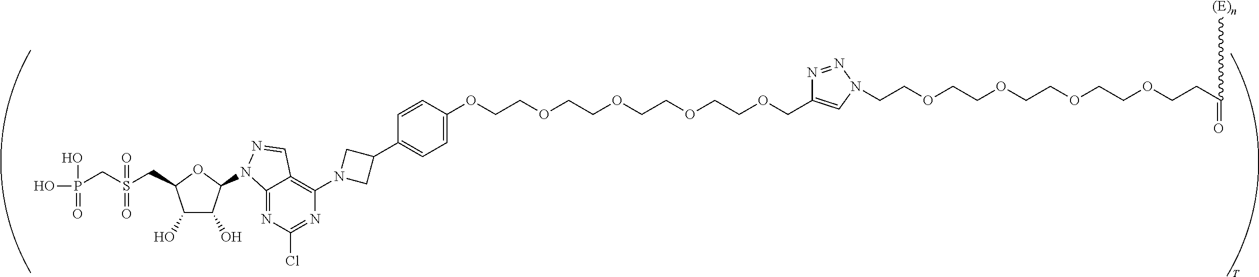 |
| Int- 308 | 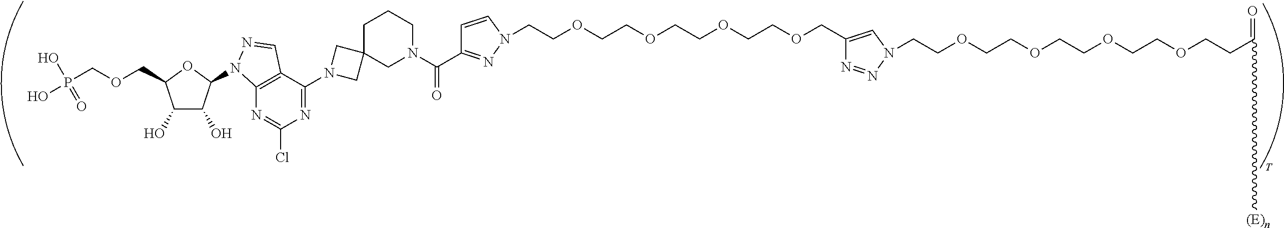 |
| Int- 309 | 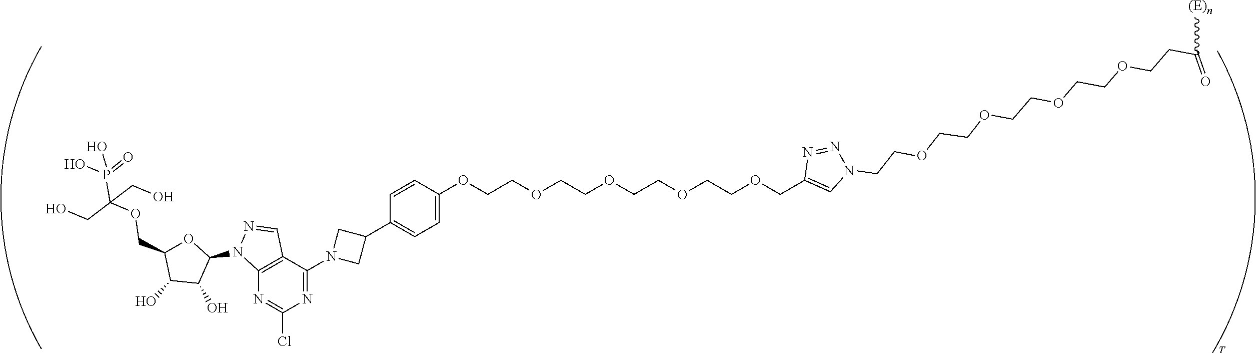 |
| Int- 310 | 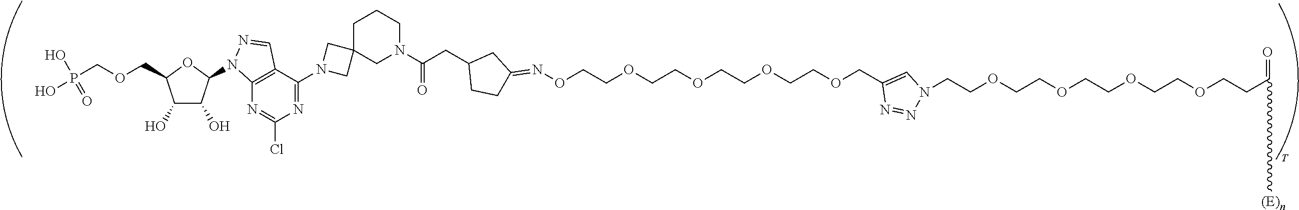 |
| Int- 311 | 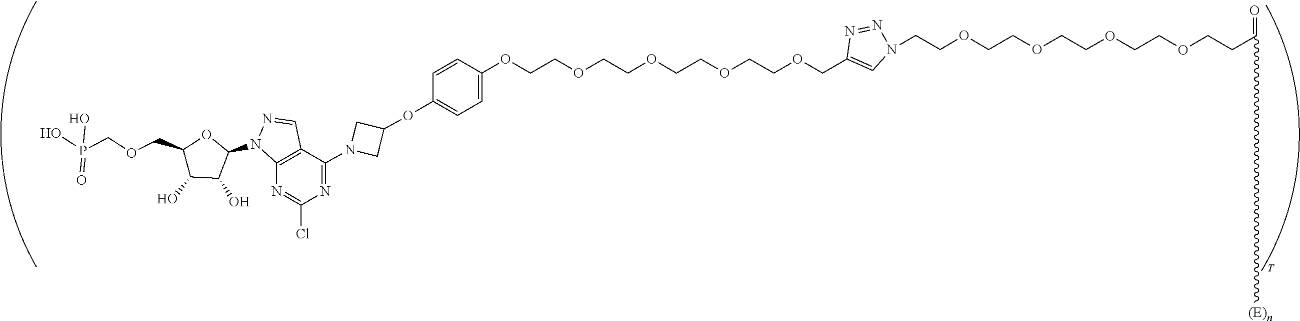 |
| Int- 312 | 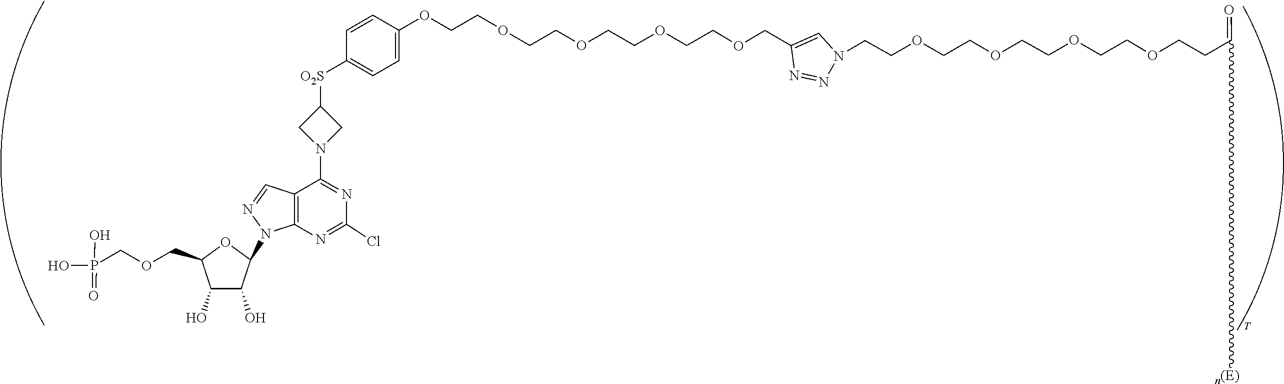 |
| Int- 313 | 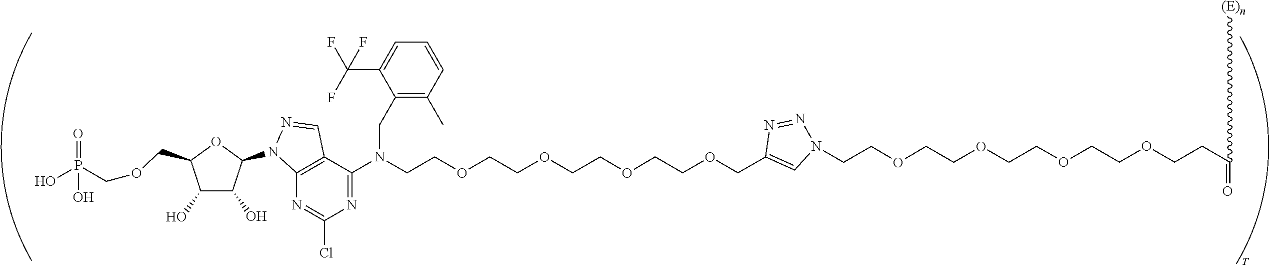 |
| Int- 314 | 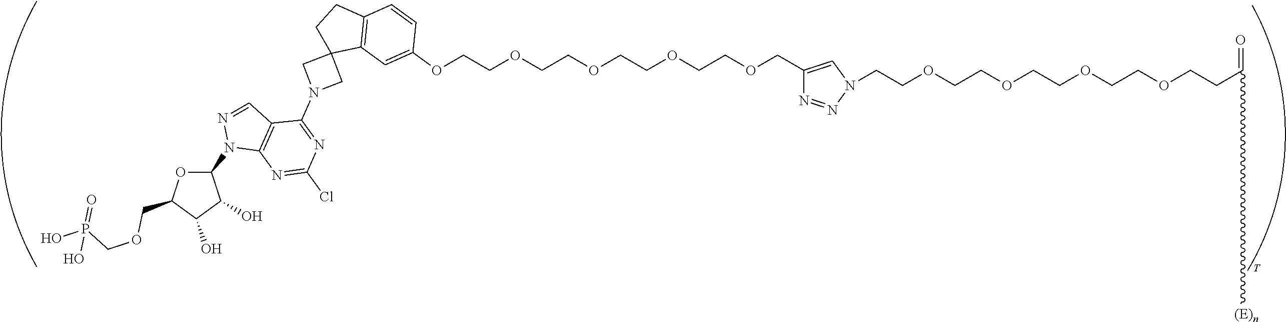 |
| Int- 315 | 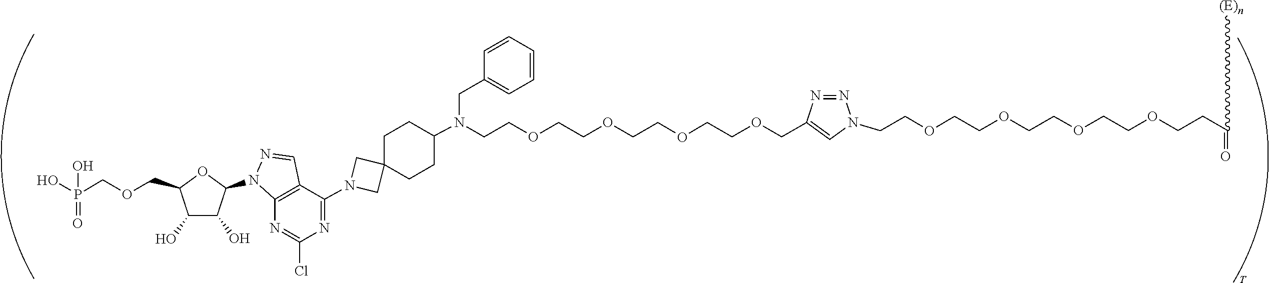 |
| Int- 316 | 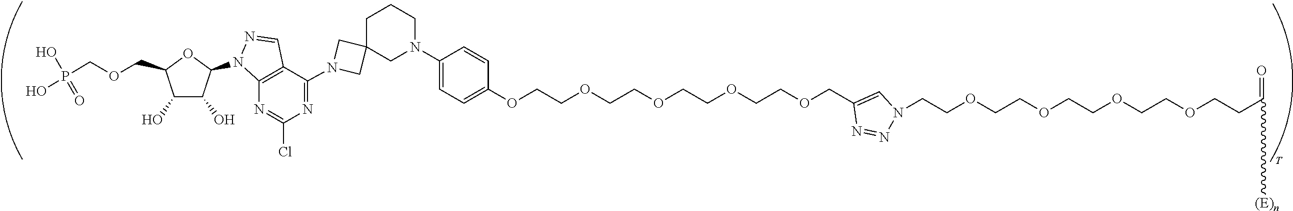 |
| Int- 317 | 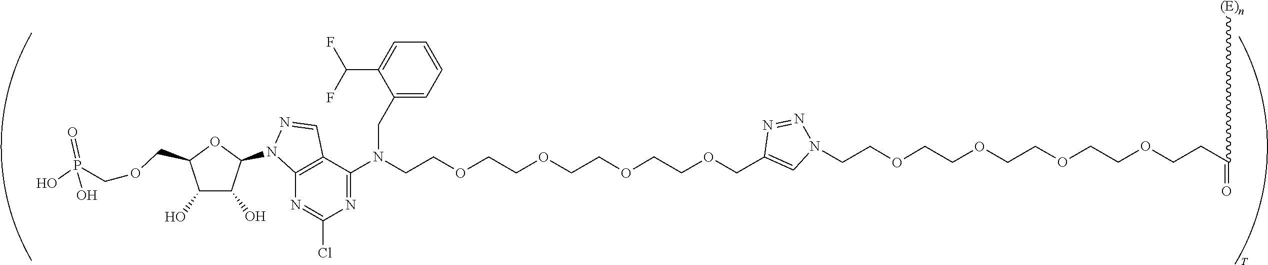 |
| Int- 318 | 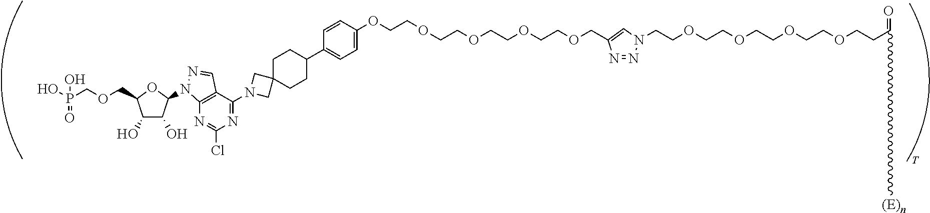 |
| Int- 320 | 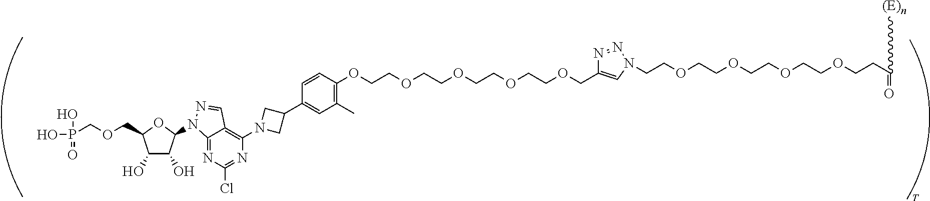 |
| Int- 321 | 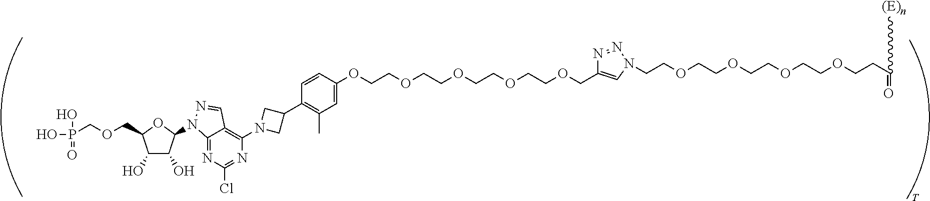 |
| Int- 322 | 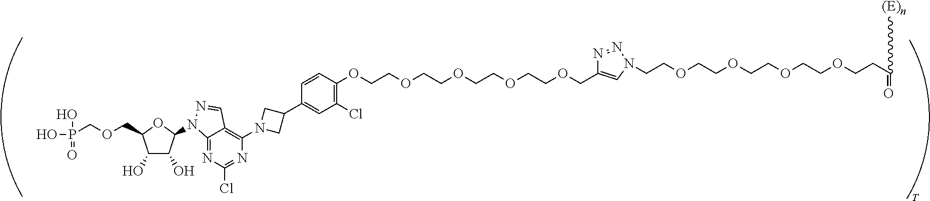 |
| Int- 323 | 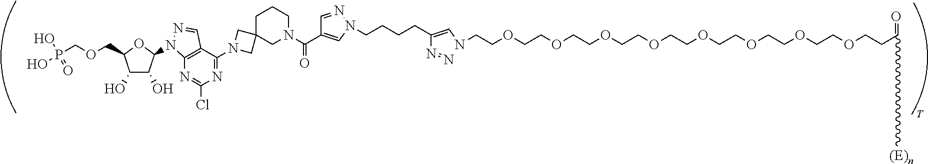 |
| Int- 324 | 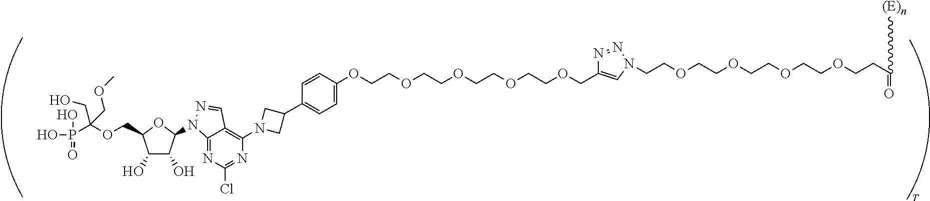 |
| Int- 325 | 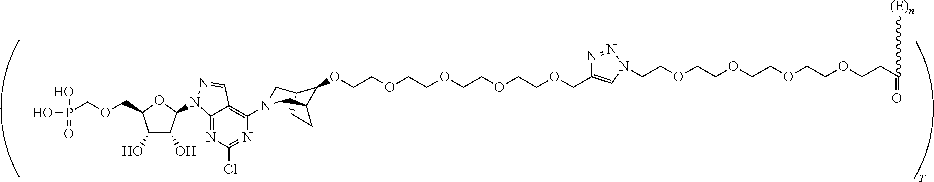 |
| Int- 326 | 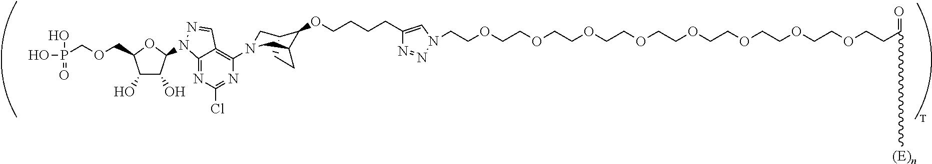 |
| Int- 327 | 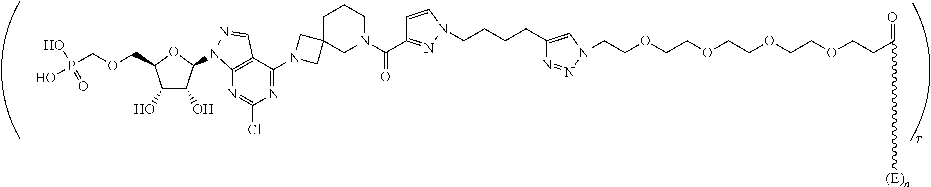 |
| Int- 328 | 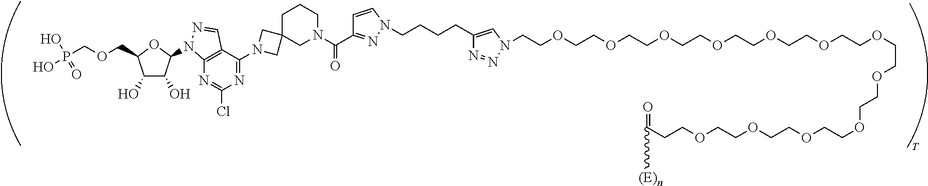 |
| Int- 329 | 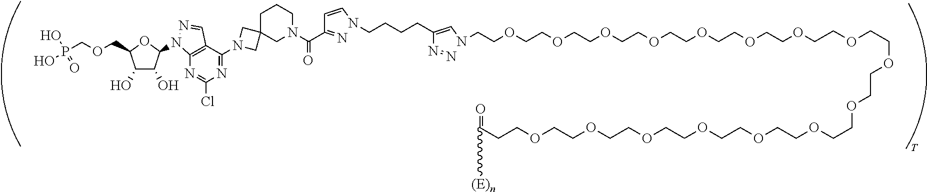 |
| Int- 330 | 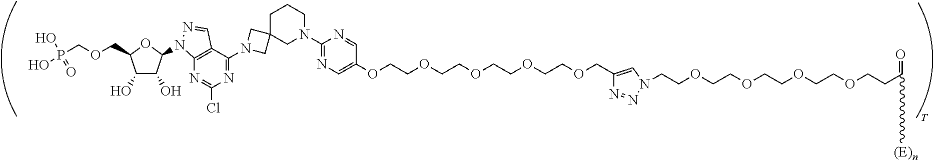 |
| Int- 333 | 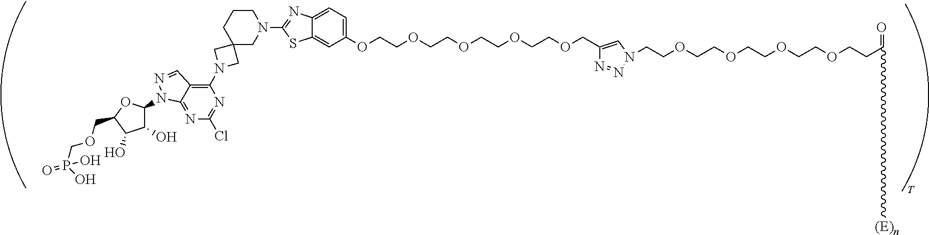 |
| Int- 334 | 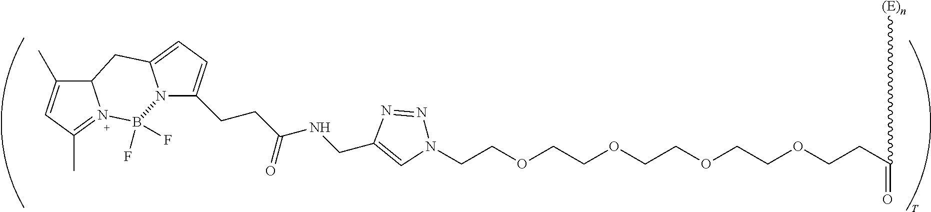 |
| Int- 335 | 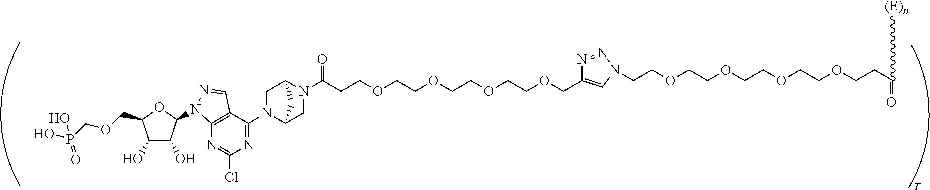 |

The disclosure further features a method of making an Fc conjugate by conjugating (e.g., via a linker) an Int of Table 1 to an Fc domain monomer or an Fc domain. In some embodiments, the disclosure provides a conjugate, wherein the conjugate includes a small molecule targeting agent, wherein the targeting agent is described by an Int of Table 1, which is conjugated to an Fc (e.g., via a linker).

In some embodiments, the squiggly line connected to E indicates that the L of each A₁-L or each A₁-L-A₂ is covalently attached to a nitrogen atom of a solvent-exposed lysine of E. In some embodiments, the squiggly line connected to E indicates that the L of each A₁-L or each A₁-L-A₂ is covalently attached to the sulfur atom of a solvent-exposed cysteine of E.

In some embodiments, n is 2, and each E dimerizes to form an Fc domain.

In some embodiments, each E is a human IgG1 Fc domain monomer. In some embodiments, each E includes a substitution mutation at N297 selected from N297A, N297G, or N297Q, wherein the amino acid numbering of each Fc domain monomer is according to the Kabat EU index. In some embodiments, each E includes a C220S substitution mutation, wherein the amino acid numbering of each Fc domain monomer is according to the Kabat EU index. In some embodiments, each E includes a M252Y, a S254T, and a T256E substitution mutation, wherein the amino acid numbering of each Fc domain monomer is according to the Kabat EU index.

In some embodiments, each E includes an amino acid sequence having at least 85%, 90%, 95%, 98%, or 99% sequence identity to the amino acid sequence of any one of SEQ ID NOs: 1-112 and 115-120. In some embodiments, each E includes the amino acid sequence of any one of SEQ ID NOs: 1-112 and 115-120.

In some embodiments, each E includes an amino acid sequence having at least 85%, 90%, 95%, 98%, or 99% sequence identity to the amino acid sequence of any one of SEQ ID NO: 13, SEQ ID NO: 14, SEQ ID NO: 17, or SEQ ID NO: 18. In some embodiments, each E includes the amino acid sequence of any one of SEQ ID NO: 13, SEQ ID NO: 14, SEQ ID NO: 17, or SEQ ID NO: 18. In some embodiments, each E includes the amino acid sequence of SEQ ID NO: 13. In some embodiments, each E includes the amino acid sequence of SEQ ID NO: 14. In some embodiments, each E includes the amino acid sequence of SEQ ID NO: 17. In some embodiments, each E includes the amino acid sequence of SEQ ID NO: 18.

In some embodiments, each E includes an amino acid sequence having at least 85%, 90%, 95%, 98%, or 99% sequence identity to the amino acid sequence of any one of SEQ ID NO: 80, SEQ ID NO: 81, SEQ ID NO: 82, or SEQ ID NO: 83. In some embodiments, each E includes the amino acid sequence of any one of SEQ ID NO: 80, SEQ ID NO: 81, SEQ ID NO: 82, or SEQ ID NO: 83. In some embodiments, each E includes the amino acid sequence of SEQ ID NO: 80. In some embodiments, each E includes the amino acid sequence of SEQ ID NO: 81. In some embodiments, each E includes the amino acid sequence of SEQ ID NO: 82. In some embodiments, each E includes the amino acid sequence of SEQ ID NO: 83.

In some embodiments, each E includes an amino acid sequence having at least 85%, 90%, 95%, 98%, or 99% sequence identity to the amino acid sequence of SEQ ID NO: 115. In some embodiments, each E includes the amino acid sequence of SEQ ID NO: 115. In some embodiments, each E includes an amino acid sequence having at least 85%, 90%, 95%, 98%, or 99% sequence identity to the amino acid sequence of SEQ ID NO: 116. In some embodiments, each E includes the amino acid sequence of SEQ ID NO: 116. In some embodiments, each E includes an amino acid sequence having at least 85%, 90%, 95%, 98%, or 99% sequence identity to the amino acid sequence of SEQ ID NO: 117. In some embodiments, each E includes the amino acid sequence of SEQ ID NO: 117. In some embodiments, each E includes an amino acid sequence having at least 85%, 90%, 95%, 98%, or 99% sequence identity to the amino acid sequence of SEQ ID NO: 118. In some embodiments, each E includes the amino acid sequence of SEQ ID NO: 118. In some embodiments, each E includes an amino acid sequence having at least 85%, 90%, 95%, 98%, or 99% sequence identity to the amino acid sequence of SEQ ID NO: 119. In some embodiments, each E includes the amino acid sequence of SEQ ID NO: 119. In some embodiments, each E includes an amino acid sequence having at least 85%, 90%, 95%, 98%, or 99% sequence identity to the amino acid sequence of SEQ ID NO: 120. In some embodiments, each E includes the amino acid sequence of SEQ ID NO: 120.

In some embodiments, n is 1 and T represents the number of A₁-L or A₁-L-A₂ moieties bound to each E. In some embodiments, n is 2 and the two Es dimerize to form a Fc domain and T represents the number of A₁-L or A₁-L-A₂ moieties bound to the Fc domain. In some embodiments, T is an integer from 1 to 20 (e.g., 1, 2, 3, 4, 5, 6, 7, 8, 9, 10, 11, 12, 13, 14, 15, 16, 17, 18, 19, or 20).

The disclosure also provides a population of conjugates described herein wherein the average value of T is 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20.

In another aspect, the disclosure provides a pharmaceutical composition including a conjugate or a population of conjugates, or a pharmaceutically acceptable salt thereof, described herein and a pharmaceutically acceptable excipient.

In another aspect, the disclosure provides a cancer in a subject, the method including administering to the subject a conjugate, population of conjugates, or pharmaceutical composition described herein.

In some embodiments, the cancer is selected from lung cancer, optionally non-small cell lung cancer or small-cell lung cancer; head and neck cancer, optionally squamous cell carcinoma; renal cell carcinoma; breast cancer; ovarian cancer; pancreatic cancer; colorectal cancer; urothelial cancer; bile duct cancer; endometrial cancer; melanoma; or esophageal cancer. In some embodiments, the cancer is a solid tumor. In some embodiments, the cancer overexpresses or is known to overexpress CD73 relative to a non-cancerous cell of the same tissue type.

In some embodiments, the method further includes administering to the subject an immune checkpoint inhibitor. In some embodiments, the immune checkpoint inhibitor is a PD-1 inhibitor. In some embodiments, the PD-1 inhibitor is an anti-PD-1 monoclonal antibody.

In another aspect, the disclosure provides a method of treating or preventing a viral infection in a subject, the method including administering to the subject a conjugate, population of conjugates, or pharmaceutical composition described herein. In some embodiments, the viral infection is a betacoronavirus infection. In some embodiments, the betacoronavirus is SARS-CoV-2. In some embodiments, the SARS-CoV-2 is an Alpha, Delta, or Omicron variant. In some embodiments, the SARS-CoV-2 is an Omicron variant. In some embodiments, the Omicron variant is a BA.1, BA.2, BA.3, BA.4, or BA.5 lineage. In some embodiments, the method further includes administering to the subject an antiviral agent or an antiviral vaccine.

In another aspect, the disclosure provides a method of treating or preventing fibrosis in a subject, the method including administering to the subject a conjugate, population of conjugates, or pharmaceutical composition described herein. In some embodiments, the fibrosis is pulmonary fibrosis, dermal fibrosis, renal fibrosis, hepatic fibrosis, cardiac fibrosis, or systemic sclerosis. In some embodiments, the fibrosis is pulmonary fibrosis. In some embodiments, the pulmonary fibrosis associated with a viral infection (e.g., associated with a SARS-CoV-2 infection), drug-induced pulmonary fibrosis, radiation-induced pulmonary fibrosis, hypersensitivity pneumonitis, idiopathic pulmonary fibrosis, non-specific interstitial pneumonia, pneumoconiosis, interstitial lung disease, sarcoidosis, silicosis, or systemic sclerosis.

In some embodiments of any of the methods of treatment described herein, the conjugate, population of conjugates, or pharmaceutical composition is administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally, locally, by inhalation, by injection, or by infusion.

### Definitions

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity but include the general class of which a specific example may be used for illustration. The terminology herein is used to describe specific embodiments of the invention, but their usage does not delimit the invention, except as outlined in the claims.

As used herein, the term “Fc domain monomer” refers to a polypeptide chain that includes at least a hinge domain and second and third antibody constant domains (C_{H2} and C_{H}3) or functional fragments thereof (e.g., fragments that that capable of (i) dimerizing with another Fc domain monomer to form an Fc domain, and (ii) binding to an Fc receptor. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., IgG1). In some embodiments, an Fc domain monomer does not include any portion of an immunoglobulin that is capable of acting as an antigen-recognition region, e.g., a variable domain or a complementarity determining region (CDR). Fc domain monomers in the compositions as described herein can contain one or more changes from a wild-type Fc domain monomer sequence (e.g., 1-10, 1-8, 1-6, 1-4 amino acid substitutions, additions, or deletions) that alter the interaction between an Fc domain and an Fc receptor. Examples of suitable changes are known in the art. In certain embodiments, a human Fc domain monomer (e.g., an IgG heavy chain, such as IgG1) includes a region that extends from any of Asn208, Glu216, Asp221, Lys222, or Cys226 to the carboxyl-terminus of the heavy chain at Lys447. C-terminal Lys447 of the Fc region may or may not be present, without affecting the structure or stability of the Fc region. C-terminal Lys 447 may be proteolytically cleaved upon expression of the polypeptide. In some embodiments of any of the Fc domain monomers described herein, C-terminal Lys 447 is optionally present or absent. The N-terminal N (e.g., Asn 201) of the Fc region may or may not be present, without affecting the structure of stability of the Fc region. N-terminal Asn may be deamidated upon expression of the polypeptide. In some embodiments of any of the Fc domain monomers described herein, N-terminal Asn is optionally present or absent. Unless otherwise specified herein, numbering of amino acid residues in the IgG or Fc domain monomer is according to the EU numbering system for antibodies, also called the Kabat EU index, as described, for example, in Kabat et al., Sequences of Proteins of Immunological Interest, 5th Ed. Public Health Service, National Institutes of Health, Bethesda, MD, 1991.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, in some embodiments, one or more disulfide bonds form between the hinge domains of the two dimerizing Fc domain monomers.

The terms “Fab” or “fragment antigen-binding,” as used interchangeably herein, refer to a region on an antibody that binds to an antigen. Fab is a term of art and its meaning is known to those of skill in the art. A Fab region is composed of one constant and one variable domain of each of the heavy and light chain. Each heavy chain is comprised of a heavy chain variable region (VH) and a heavy chain constant region (CH). The heavy chain constant region may be comprised of three domains, CH₁, CH₂, and/or CH₃. Each light chain is comprised of a light chain variable region (VL) and a light chain constant region (CL). The VH and VL regions can be further subdivided into regions of hypervariability, termed “complementarity determining regions” (CDRs), interspersed with regions that are more conserved, termed “framework regions” (FRs). In antibodies, the heavy chain (e.g., the VH and CH region) is linked to the Fc domain monomer by way of a hinge. The Fc domain monomers described herein may include between 10 and/or 20 residues (e.g., 11, 12, 13, 14, 15, 16, 17, 18, or 19 residues) of the Fab domain and hinge region. In certain embodiments, the N-terminus of the Fc domain monomer is any one of amino acid residues 198-205 (corresponding to a residue of the Fab domain). In some embodiments, the N-terminus of the Fc domain monomer is amino acid residue 201 (e.g., Asn 201). In certain embodiments, the N-terminus of the Fc domain monomer is amino acid residue 202 (e.g., Val 202).

The term “covalently attached” refers to two parts of a conjugate that are linked to each other by a covalent bond formed between two atoms in the two parts of the conjugate.

As used-herein, a “surface exposed amino acid” or “solvent-exposed amino acid,” such as a surface exposed cysteine or a surface exposed lysine refers to an amino acid that is accessible to the solvent surrounding the protein. A surface exposed amino acid may be a naturally occurring or an engineered variant (e.g., a substitution or insertion) of the protein. In some embodiments, a surface exposed amino acid is an amino acid that when substituted does not substantially change the three-dimensional structure of the protein.

The term “linker” as used herein, refer to a covalent linkage or connection between two or more components in a conjugate (e.g., between two CD73 inhibitors in a conjugate described herein, between a CD73 inhibitor and an Fc domain in a conjugate described herein, and between a dimer of two CD73 inhibitors and an Fc domain in a conjugate described herein). In some embodiments, a conjugate described herein may contain a linker that has a bivalent structure (e.g., a bivalent linker). A bivalent linker has two arms, in which each arm is covalently linked to a component of the conjugate (e.g., a first arm conjugated to a CD73 inhibitor and a second arm conjugated to an Fc domain). In some embodiments, a conjugate described herein may contain a linker that has a trivalent structure (e.g., a trivalent linker). A trivalent linker has three arms, in which each arm is covalently linked to a component of the conjugate (e.g., a first arm conjugated to a CD73 inhibitor, a second arm conjugated to a second CD73 inhibitor, and a third arm conjugated to an Fc domain). Linkers of the disclosure may be linear or branched.

In some embodiments, molecules that may be used as linkers include at least two functional groups, which may be the same or different, e.g., two carboxylic acid groups, two amine groups, two sulfonic acid groups, a carboxylic acid group and a maleimide group, a carboxylic acid group and an alkyne group, a carboxylic acid group and an amine group, a carboxylic acid group and a sulfonic acid group, an amine group and a maleimide group, an amine group and an alkyne group, or an amine group and a sulfonic acid group. The first functional group may form a covalent linkage with a first component in the conjugate and the second functional group may form a covalent linkage with the second component in the conjugate. In some embodiments of a trivalent linker, two arms of a linker may contain two dicarboxylic acids, in which the first carboxylic acid may form a covalent linkage with the first CD73 inhibitor in the conjugate and the second carboxylic acid may form a covalent linkage with the second CD73 inhibitor in the conjugate, and the third arm of the linker may for a covalent linkage with an Fc domain in the conjugate. Examples of dicarboxylic acids are described further herein. In some embodiments, a molecule containing one or more maleimide groups may be used as a linker, in which the maleimide group may form a carbon-sulfur linkage with a cysteine in a component (e.g., an Fc domain) in the conjugate. In some embodiments, a molecule containing one or more alkyne groups may be used as a linker, in which the alkyne group may form a 1,2,3-triazole linkage with an azide in a component (e.g., an Fc domain) in the conjugate. In some embodiments, a molecule containing one or more azide groups may be used as a linker, in which the azide group may form a 1,2,3-triazole linkage with an alkyne in a component (e.g., an Fc domain) in the conjugate. In some embodiments, a molecule containing one or more bis-sulfone groups may be used as a linker, in which the bis-sulfone group may form a linkage with an amine group a component (e.g., an Fc domain) in the conjugate. In some embodiments, a molecule containing one or more sulfonic acid groups may be used as a linker, in which the sulfonic acid group may form a sulfonamide linkage with a component in the conjugate. In some embodiments, a molecule containing one or more isocyanate groups may be used as a linker, in which the isocyanate group may form a urea linkage with a component in the conjugate. In some embodiments, a molecule containing one or more haloalkyl groups may be used as a linker, in which the haloalkyl group may form a covalent linkage, e.g., C—N and C—O linkages, with a component in the conjugate. In some embodiments, a molecule containing one or more phenyl ester groups (e.g., trifluorophenyl ester groups or tetrafluorophenyl ester groups) may be used as a linker, in which the phenyl ester group (e.g., trifluorophenyl ester group or tetrafluorophenyl ester group) may form an amide with an amine in a component (e.g., a fusion protein) in the conjugate.

In some embodiments, a linker provides space, rigidity, and/or flexibility between the two or more components. In some embodiments, a linker may be a bond, e.g., a covalent bond. The term “bond” refers to a chemical bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms. In some embodiments, a linker includes no more than 250 non-hydrogen atoms. In some embodiments, the backbone of a linker includes no more than 250 atoms. The “backbone” of a linker refers to the atoms in the linker that together form the shortest path from one part of a conjugate to another part of the conjugate (e.g., the shortest path linking a first CD73 inhibitor and a second CD73 inhibitor). The atoms in the backbone of the linker are directly involved in linking one part of a conjugate to another part of the conjugate (e.g., linking a first CD73 inhibitor and a second CD73 inhibitor). For example, hydrogen atoms attached to carbons in the backbone of the linker are not considered as directly involved in linking one part of the conjugate to another part of the conjugate.

In some embodiments, a linker may include a synthetic group derived from, e.g., a synthetic polymer (e.g., a polyethylene glycol (PEG) polymer). In some embodiments, a linker may include one or more amino acid residues, such as D- or L-amino acid residues. In some embodiments, a linker may be a residue of an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may include one or more, e.g., 1-100, 1-50, 1-25, 1-10, 1-5, or 1-3, optionally substituted alkylene, optionally substituted heteroalkylene (e.g., a PEG unit), optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted cycloalkenylene, optionally substituted heterocycloalkenylene, optionally substituted cycloalkynylene, optionally substituted heterocycloalkynylene, optionally substituted arylene, optionally substituted heteroarylene (e.g., pyridine), O, S, NRⁱ (Rⁱ is H, optionally substituted alkyl, optionally substituted heteroalkyl, optionally substituted alkenyl, optionally substituted heteroalkenyl, optionally substituted alkynyl, optionally substituted heteroalkynyl, optionally substituted cycloalkyl, optionally substituted heterocycloalkyl, optionally substituted cycloalkenyl, optionally substituted heterocycloalkenyl, optionally substituted cycloalkynyl, optionally substituted heterocycloalkynyl, optionally substituted aryl, or optionally substituted heteroaryl), P, carbonyl, thiocarbonyl, sulfonyl, phosphate, phosphoryl, or imino. For example, a linker may include one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C2-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (R¹ is H, optionally substituted C₁-C₂₀ alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

The terms “alkyl,” “alkenyl,” and “alkynyl,” as used herein, include straight-chain and branched-chain monovalent substituents, as well as combinations of these, containing only C and H when unsubstituted. When the alkyl group includes at least one carbon-carbon double bond or carbon-carbon triple bond, the alkyl group can be referred to as an “alkenyl” or “alkynyl” group respectively. The monovalency of an alkyl, alkenyl, or alkynyl group does not include the optional substituents on the alkyl, alkenyl, or alkynyl group. For example, if an alkyl, alkenyl, or alkynyl group is attached to a compound, monovalency of the alkyl, alkenyl, or alkynyl group refers to its attachment to the compound and does not include any additional substituents that may be present on the alkyl, alkenyl, or alkynyl group. In some embodiments, the alkyl or heteroalkyl group may contain, e.g., 1-20. 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, the alkenyl, heteroalkenyl, alkynyl, or heteroalkynyl group may contain, e.g., 2-20, 2-18, 2-16, 2-14, 2-12, 2-10, 2-8, 2-6, or 2-4 carbon atoms (e.g., C2-C20, C2-C18, C2-C16, C2-C14, C2-C12, C2-C10, C2-C8, C2-C6, or C2-C4). Examples include, but are not limited to, methyl, ethyl, isobutyl, sec-butyl, tert-butyl, 2-propenyl, and 3-butynyl. A heterocycloalkyl, heterocycloalkenyl, or heterocycloalkynyl group refers to a cycloalkyl, cycloalkenyl, or cycloalkynyl group that has one or more heteroatoms independently selected from, e.g., N, O, and S. Exemplary heterocycloalkyl groups include pyrrolidine, thiophene, thiolane, tetrahydrofuran, piperidine, and tetrahydropyran.

The term “cycloalkyl,” as used herein, represents a monovalent saturated or unsaturated non-aromatic cyclic alkyl group. A cycloalkyl may have, e.g., three to twenty carbons (e.g., a C3-C7, C₃-C₈, C3-C9, C3-C10, C3-C11, C3-C12, C3-C14, C3-C16, C3-C18, or C3-C20 cycloalkyl). Examples of cycloalkyls include, but are not limited to, cyclopropyl, cyclobutyl, cyclopentyl, cyclohexyl, and cycloheptyl.

When the cycloalkyl group includes at least one carbon-carbon double bond, the cycloalkyl group can be referred to as a “cycloalkenyl” group. A cycloalkenyl may have, e.g., four to twenty carbons (e.g., a C4-C7, C4-C8, C4-C9, C4-C10, C4-C11, C4-C12, C4-C14, C4-C16, C4-C18, or C4-C20 cycloalkenyl). Exemplary cycloalkenyl groups include, but are not limited to, cyclopentenyl, cyclohexenyl, and cycloheptenyl. When the cycloalkyl group includes at least one carbon-carbon triple bond, the cycloalkyl group can be referred to as a “cycloalkynyl” group. A cycloalkynyl may have, e.g., eight to twenty carbons (e.g., a C8-C9, C8-C10, C8-C11, C8-C12, C8-C14, C8-C16, C8-C18, or C8-C20 cycloalkynyl). The term “cycloalkyl” also includes a cyclic compound having a bridged multicyclic structure in which one or more carbons bridges two non-adjacent members of a monocyclic ring, e.g., bicyclo[2.2.1.]heptyl and adamantane. The term “cycloalkyl” also includes bicyclic, tricyclic, and tetracyclic fused ring structures, e.g., decalin and spiro cyclic compounds.

The term “aryl,” as used herein, refers to any monocyclic or fused ring bicyclic or tricyclic system which has the characteristics of aromaticity in terms of electron distribution throughout the ring system, e.g., phenyl, naphthyl, or phenanthrene. In some embodiments, a ring system contains 5-15 ring member atoms or 5-10 ring member atoms. An aryl group may have, e.g., five to fifteen carbons (e.g., a C5-C6, C5-C7, C5-C8, C5-C9, C5-C10, C5-C11, C5-C12, C5-C13, C5-C14, or C5-C15 aryl). The term “heteroaryl” also refers to such monocyclic or fused bicyclic ring systems containing one or more, e.g., 1-4, 1-3, 1, 2, 3, or 4, heteroatoms selected from O, S and N. A heteroaryl group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroaryl). The inclusion of a heteroatom permits inclusion of 5-membered rings to be considered aromatic as well as 6-membered rings. Thus, typical heteroaryl systems include, e.g., pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, and imidazolyl. Because tautomers are possible, a group such as phthalimido is also considered heteroaryl. In some embodiments, the aryl or heteroaryl group is a 5- or 6-membered aromatic rings system optionally containing 1-2 nitrogen atoms. In some embodiments, the aryl or heteroaryl group is an optionally substituted phenyl, pyridyl, indolyl, pyrimidyl, pyridazinyl, benzothiazolyl, benzimidazolyl, pyrazolyl, imidazolyl, isoxazolyl, thiazolyl, or imidazopyridinyl. In some embodiments, the aryl group is phenyl. In some embodiments, an aryl group may be optionally substituted with a substituent such an aryl substituent, e.g., biphenyl.

The term “alkaryl,” refers to an aryl group that is connected to an alkylene, alkenylene, or alkynylene group. In general, if a compound is attached to an alkaryl group, the alkylene, alkenylene, or alkynylene portion of the alkaryl is attached to the compound. In some embodiments, an alkaryl is C6-C35 alkaryl (e.g., C6-C16, C6-C14, C6-C12, C6-C10, C6-C9, C6-C8, C7, or C6 alkaryl), in which the number of carbons indicates the total number of carbons in both the aryl portion and the alkylene, alkenylene, or alkynylene portion of the alkaryl. Examples of alkaryls include, but are not limited to, (C1-C8)alkylene(C6-C12)aryl, (C2-C8)alkenylene(C6-C12)aryl, or (C2-C8)alkynylene(C6-C12)aryl. In some embodiments, an alkaryl is benzyl or phenethyl. In a heteroalkaryl, one or more heteroatoms selected from N, O, and S may be present in the alkylene, alkenylene, or alkynylene portion of the alkaryl group and/or may be present in the aryl portion of the alkaryl group. In an optionally substituted alkaryl, the substituent may be present on the alkylene, alkenylene, or alkynylene portion of the alkaryl group and/or may be present on the aryl portion of the alkaryl group.

The term “amino,” as used herein, represents —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino group is —NH₂.

The term “alkamino,” as used herein, refers to an amino group, described herein, that is attached to an alkylene (e.g., C1-C5 alkylene), alkenylene (e.g., C2-C5 alkenylene), or alkynylene group (e.g., C2-C5 alkenylene). In general, if a compound is attached to an alkamino group, the alkylene, alkenylene, or alkynylene portion of the alkamino is attached to the compound. The amino portion of an alkamino refers to —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino portion of an alkamino is —NH₂. An example of an alkamino group is C1-C5 alkamino, e.g., C2 alkamino (e.g., CH₂CH₂NH₂ or CH₂CH₂N(CH₃)₂). In a heteroalkamino group, one or more, e.g., 1-4, 1-3, 1, 2, 3, or 4, heteroatoms selected from N, O, and S may be present in the alkylene, alkenylene, or alkynylene portion of the heteroalkamino group. In some embodiments, an alkamino group may be optionally substituted. In a substituted alkamino group, the substituent may be present on the alkylene, alkenylene, or alkynylene portion of the alkamino group and/or may be present on the amino portion of the alkamino group.

The term “alkamide,” as used herein, refers to an amide group that is attached to an alkylene (e.g., C1-C5 alkylene), alkenylene (e.g., C2-C5 alkenylene), or alkynylene (e.g., C2-C5 alkenylene) group. In general, if a compound is attached to an alkamide group, the alkylene, alkenylene, or alkynylene portion of the alkamide is attached to the compound. The amide portion of an alkamide refers to —C(O)—N(R^{x})₂, where each R^{x} is, independently, H, alkyl, alkenyl, alkynyl, aryl, alkaryl, cycloalkyl, or two R^{x} combine to form a heterocycloalkyl. In some embodiment, the amide portion of an alkamide is —C(O)NH₂. An alkamide group may be —(CH₂)₂—C(O)NH₂ or —CH₂—C(O)NH₂. In a heteroalkamide group, one or more, e.g., 1-4, 1-3, 1, 2, 3, or 4, heteroatoms selected from N, O, and S may be present in the alkylene, alkenylene, or alkynylene portion of the heteroalkamide group. In some embodiments, an alkamide group may be optionally substituted. In a substituted alkamide group, the substituent may be present on the alkylene, alkenylene, or alkynylene portion of the alkamide group and/or may be present on the amide portion of the alkamide group.

The terms “alkylene,” “alkenylene,” and “alkynylene,” as used herein, refer to divalent groups having a specified size. In some embodiments, an alkylene may contain, e.g., 1-20, 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, an alkenylene or alkynylene may contain, e.g., 2-20, 2-18, 2-16, 2-14, 2-12, 2-10, 2-8, 2-6, or 2-4 carbon atoms (e.g., C2-C20, C2-C18, C2-C16, C2-C14, C2-C12, C2-C10, C2-C8, C2-C6, or C2-C4). Alkylene, alkenylene, and/or alkynylene includes straight-chain and branched-chain forms, as well as combinations of these. The divalency of an alkylene, alkenylene, or alkynylene group does not include the optional substituents on the alkylene, alkenylene, or alkynylene group. For example, two CD73 inhibitors may be attached to each other by way of a linker that includes alkylene, alkenylene, and/or alkynylene, or combinations thereof. Each of the alkylene, alkenylene, and/or alkynylene groups in the linker is considered divalent with respect to the two attachments on either end of alkylene, alkenylene, and/or alkynylene group. For example, if a linker includes -(optionally substituted alkylene)-(optionally substituted alkenylene)-(optionally substituted alkylene)-, the alkenylene is considered divalent with respect to its attachments to the two alkylenes at the ends of the linker. The optional substituents on the alkenylene are not included in the divalency of the alkenylene. The divalent nature of an alkylene, alkenylene, or alkynylene group (e.g., an alkylene, alkenylene, or alkynylene group in a linker) refers to both of the ends of the group and does not include optional substituents that may be present in an alkylene, alkenylene, or alkynylene group. Because they are divalent, they can link together multiple (e.g., two) parts of a conjugate, e.g., a first CD73 inhibitor and a second CD73 inhibitor. Alkylene, alkenylene, and/or alkynylene groups can be substituted by the groups typically suitable as substituents for alkyl, alkenyl and alkynyl groups as set forth herein. For example, C═O is a C1 alkylene that is substituted by an oxo (=O). For example, —HCR—C≡C— may be considered as an optionally substituted alkynylene and is considered a divalent group even though it has an optional substituent, R. Heteroalkylene, heteroalkenylene, and/or heteroalkynylene groups refer to alkylene, alkenylene, and/or alkynylene groups including one or more, e.g., 1-4, 1-3, 1, 2, 3, or 4, heteroatoms, e.g., N, O, and S. For example, a polyethylene glycol (PEG) polymer or a PEG unit —(CH₂)₂—O— in a PEG polymer is considered a heteroalkylene containing one or more oxygen atoms.

The term “cycloalkylene,” as used herein, refers to a divalent cyclic group linking together two parts of a compound. For example, one carbon within the cycloalkylene group may be linked to one part of the compound, while another carbon within the cycloalkylene group may be linked to another part of the compound. A cycloalkylene group may include saturated or unsaturated non-aromatic cyclic groups. A cycloalkylene may have, e.g., three to twenty carbons in the cyclic portion of the cycloalkylene (e.g., a C3-C7, C3-C8, C3-C9, C3-C10, C3-C11, C3-C12, C3-C14, C3-C16, C3-C18, or C3-C20 cycloalkylene). When the cycloalkylene group includes at least one carbon-carbon double bond, the cycloalkylene group can be referred to as a “cycloalkenylene” group. A cycloalkenylene may have, e.g., four to twenty carbons in the cyclic portion of the cycloalkenylene (e.g., a C4-C7, C4-C8, C4-C9. C4-C10, C4-C11, C4-C12, C4-C14, C4-C16, C4-C18, or C4-C20 cycloalkenylene). When the cycloalkylene group includes at least one carbon-carbon triple bond, the cycloalkylene group can be referred to as a “cycloalkynylene” group. A cycloalkynylene may have, e.g., four to twenty carbons in the cyclic portion of the cycloalkynylene (e.g., a C4-C7, C4-C8, C4-C9. C4-C10, C4-C11, C4-C12, C4-C14, C4-C16, C4-C18, or C8-C20 cycloalkynylene). A cycloalkylene group can be substituted by the groups typically suitable as substituents for alkyl, alkenyl and alkynyl groups as set forth herein. Heterocycloalkylene refers to a cycloalkylene group including one or more, e.g., 1-4, 1-3, 1, 2, 3, or 4, heteroatoms, e.g., N, O, and S. Examples of cycloalkylenes include, but are not limited to, cyclopropylene and cyclobutylene. A tetrahydrofuran may be considered as a heterocycloalkylene.

The term “arylene,” as used herein, refers to a multivalent (e.g., divalent or trivalent) aryl group linking together multiple (e.g., two or three) parts of a compound. For example, one carbon within the arylene group may be linked to one part of the compound, while another carbon within the arylene group may be linked to another part of the compound. An arylene may have, e.g., five to fifteen carbons in the aryl portion of the arylene (e.g., a C5-C6, C5-C7, C5-C8, C5-C9. C5-C10, C5-C11, C5-C12, C5-C13, C5-C14, or C5-C15 arylene). An arylene group can be substituted by the groups typically suitable as substituents for alkyl, alkenyl and alkynyl groups as set forth herein. Heteroarylene refers to an aromatic group including one or more, e.g., 1-4, 1-3, 1, 2, 3, or 4, heteroatoms, e.g., N, O, and S. A heteroarylene group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroarylene).

The term “optionally substituted,” as used herein, refers to having 0, 1, or more substituents, such as 0-25, 0-20, 0-10 or 0-5 substituents. Alkyl, heteroalkyl, alkoxyl, cycloalkyl, heterocyclyl, aryl, and heteroaryl may be substituted with alkyl, halogen, alkenyl, alkynyl, aryl, alkaryl, acyl, heteroaryl, heteroalkyl, heteroalkenyl, heteroalkynyl, heteroalkaryl, halogen, oxo, cyano, nitro, amino, alkamino, hydroxy, alkoxy, alkanoyl, carbonyl, carbamoyl, guanidinyl, ureido, amidinyl, oximo, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, cycloalkyl, heterocyclyl, alkenyl, aryl, heteroalkyl, heteroalkenyl, or heteroaryl. In some embodiments, a substituent is further substituted as described herein. For example, a C1 alkyl group, i.e., methyl, may be substituted with oxo to form a formyl group and further substituted with —OH or —NHR to form a carboxyl group or an amido group.

An optionally substituted group or moiety refers to a group or moiety (e.g., any one of the groups or moieties described above) in which one of the atoms (e.g., a hydrogen atom) is optionally replaced with another substituent. For example, an optionally substituted alkyl may be an optionally substituted methyl, in which a hydrogen atom of the methyl group is replaced by, e.g., OH. As another example, a substituent on a heteroalkyl or its divalent counterpart, heteroalkylene, may replace a hydrogen on a carbon or a hydrogen on a heteroatom such as N. For example, the hydrogen atom in the group —R—NH—R— may be substituted with an alkamide substituent, e.g., —R—N[(CH₂C(O)N(CH₃)₂]—R.

Generally, an optional substituent is a noninterfering substituent. A “noninterfering substituent” refers to a substituent that leaves the ability of the conjugates described herein to either bind to CD73. Thus, in some embodiments, the substituent may alter the degree of such activity. However, as long as the conjugate retains the ability to bind to CD73 or to inhibit tumor growth, the substituent will be classified as “noninterfering.” For example, the noninterfering substituent would leave the ability of the compound to provide antiviral efficacy based on an IC50 value of 10 μM or less in a viral plaque reduction assay. Thus, the substituent may alter the degree of inhibition based on plaque reduction or CD73 inhibition. However, as long as the compounds herein retain the ability to inhibit CD73, the substituent will be classified as “noninterfering.” A number of assays for determining viral plaque reduction or tumor growth suppression or the ability of any compound to inhibit CD73 are available in the art, and some are exemplified in the Examples below.

The term “hetero,” when used to describe a chemical group or moiety, refers to having at least one heteroatom that is not a carbon or a hydrogen, e.g., N, O, and S. Any one of the groups or moieties described above may be referred to as hetero if it contains at least one heteroatom. For example, a heterocycloalkyl, heterocycloalkenyl, or heterocycloalkynyl group refers to a cycloalkyl, cycloalkenyl, or cycloalkynyl group that has one or more heteroatoms independently selected from, e.g., N, O, and S. An example of a heterocycloalkenyl group is a maleimido. For example, a heteroaryl group refers to an aromatic group that has one or more heteroatoms independently selected from, e.g., N, O, and S. One or more heteroatoms may also be included in a substituent that replaced a hydrogen atom in a group or moiety as described herein. For example, in an optionally substituted heteroaryl group, if one of the hydrogen atoms in the heteroaryl group is replaced with a substituent (e.g., methyl), the substituent may also contain one or more heteroatoms (e.g., methanol).

The term “acyl,” as used herein, refers to a group having the structure:

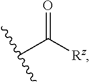

wherein R^{z} is an optionally substituted alkyl, alkenyl, alkynyl, cycloalkyl, cycloalkenyl, cycloalkynyl, aryl, alkaryl, alkamino, heteroalkyl, heteroalkenyl, heteroalkynyl, heterocycloalkyl, heterocycloalkenyl, heterocycloalkynyl, heteroaryl, heteroalkaryl, or heteroalkamino.

The term “halo” or “halogen,” as used herein, refers to any halogen atom, e.g., F, Cl, Br, or I. Any one of the groups or moieties described herein may be referred to as a “halo moiety” if it contains at least one halogen atom, such as haloalkyl.

The term “haloalkyl,” as used herein, refers to an alkyl group substituted with one or more (e.g., one, two, three, four, five, six, or more) halo groups. Haloalkyl groups include, but are not limited to, fluoroalkyl (e.g., trifluoromethyl and pentafluoroethyl) and chloroalkyl.

The term “hydroxyl,” as used herein, represents an —OH group.

The term “oxo,” as used herein, refers to a substituent having the structure ═O, where there is a double bond between an atom and an oxygen atom.

The term “carbonyl,” as used herein, refers to a group having the structure:

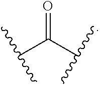 [SMILES: *C(*)=O]

The term “thiocarbonyl,” as used herein, refers to a group having the structure:

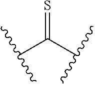 [SMILES: *C(*)=S]

The term “phosphate,” as used herein, represents the group having the structure:

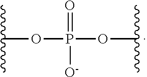 [SMILES: *OP(=O)([O-])O*]

The term “phosphoryl,” as used herein, represents the group having the structure:

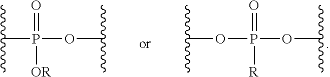

The term “sulfonyl,” as used herein, represents the group having the structure:

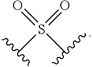 [SMILES: *S(*)(=O)=O]

The term “imino,” as used herein, represents the group including C=N (e.g., including the structure:

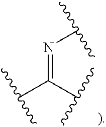 [SMILES: *N=C(*)*]

For example, an imino group may have any one of the following structures:

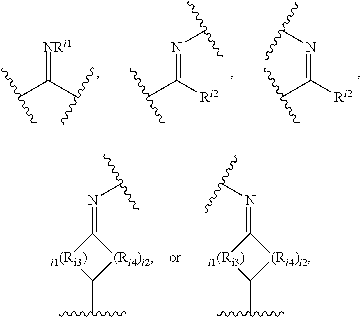

where each of Rⁱ¹ and Rⁱ² is H or any one of the substituents described herein (e.g., C₁-C₂₀ alkyl); each of Rⁱ³ and Rⁱ⁴ is a methylene that is unsubstituted or substituted with one or more of the substituents described herein (e.g., C₁-C₂₀ alkyl); and each of i1 and i2 is, independently, 0, 1, 2, or 3.

The term “oxime,” as used herein, represents the group including C═N—O (e.g., including the structure

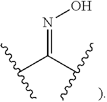 [SMILES: *C(*)=NO]

The term “N-protecting group,” as used herein, represents those groups intended to protect an amino group against undesirable reactions during synthetic procedures. Commonly used N-protecting groups are disclosed in Greene, “Protective Groups in Organic Synthesis,” 5th Edition (John Wiley & Sons, New York, 2014), which is incorporated herein by reference. N-protecting groups include, e.g., acyl, aryloyl, and carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, carboxybenzyl (CBz), 4-chlorobenzoyl, 4-bromobenzoyl, 4-nitrobenzoyl, and chiral auxiliaries such as protected or unprotected D, L or D, L-amino acid residues such as alanine, leucine, phenylalanine; sulfonyl-containing groups such as benzenesulfonyl and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl (BOC), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl (Fmoc), cyclopentyloxycarbonyl, adamantyloxycarbonyl, cyclohexyloxycarbonyl, and phenylthiocarbonyl; alkaryl groups such as benzyl, triphenylmethyl, and benzyloxymethyl; and silyl groups such as trimethylsilyl.

The term “amino acid,” as used herein, means naturally occurring amino acids and non-naturally occurring amino acids.

The term “naturally occurring amino acids,” as used herein, means amino acids including Ala, Arg, Asn, Asp, Cys, Gln, Glu, Gly, His, lie, Leu, Lys, Met, Phe, Pro, Ser, Thr, Trp, Tyr, and Val.

The term “non-naturally occurring amino acid,” as used herein, means an alpha amino acid that is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; ornithine; diaminobutyric acid; 3-aminoalanine; 3-hydroxy-D-proline; 2,4-diaminobutyric acid; 2-aminopentanoic acid; 2-aminooctanoic acid, 2-carboxy piperazine; piperazine-2-carboxylic acid, 2-amino-4-phenylbutanoic acid; 3-(2-naphthyl)alanine, and hydroxyproline. Other amino acids are α-aminobutyric acid, α-amino-α-methylbutyrate, aminocyclopropane-carboxylate, aminoisobutyric acid, aminonorbornyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-ornithine, L-N-methylethylglycine, L-norleucine, α-methyl-aminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N—(N-(2,2-diphenylethyl) carbamylmethylglycine, 1-carboxy-1-(2,2-diphenyl-ethylamino) cyclopropane, 4-hydroxyproline, ornithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, O-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)₄-hydroxypiperidine-2-carboxylic acid, (2S,4S) and (2S,4R)-4-(2-naphthylmethoxy)-pyrolidine-2-carboxylic acid, (2S,4S) and (2S,4R)₄-phenoxy-pyrrolidine-2-carboxylic acid, (2R,5S)and(2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisoleucine, L-N-methyllysine, L-N-methylnorleucine, L-N-methylornithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcylcopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cylcododecylglycine, N-cyclooctylglycine, N-cyclopropylglycine, N-cycloundecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorleucine, L-α-methylornithine, L-α-methylproline, L-α-methylthreonine, L-α-methyltyrosine, L-N-methyl-homophenylalanine, N—(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenylalanine, L-3-pyridylalanine, L-histidine (benzoyloxymethyl), N-cycloheptylglycine, L-diphenylalanine, O-methyl-L-homotyrosine, L-p-homolysine, O-glycan-threoine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(benzothiazol-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R)and (2S,4S)-4-(4-phenylbenzyl) pyrrolidine-2-carboxylic acid, (3S)-1-pyrrolidine-3-carboxylic acid, (2S,4S)-4-tritylmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine. In some embodiments, amino acid residues may be charged or polar. Charged amino acids include alanine, lysine, aspartic acid, or glutamic acid, or non-naturally occurring analogs thereof. Polar amino acids include glutamine, asparagine, histidine, serine, threonine, tyrosine, methionine, or tryptophan, or non-naturally occurring analogs thereof. It is specifically contemplated that in some embodiments, a terminal amino group in the amino acid may be an amido group or a carbamate group.

As used herein, the term “percent (%) identity” refers to the percentage of amino acid residues of a candidate sequence, e.g., an Fc-IgG, or fragment thereof, that are identical to the amino acid residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid sequence identity of a given candidate sequence to, with, or against a given reference sequence (which can alternatively be phrased as a given candidate sequence that has or includes a certain percent amino acid sequence identity to, with, or against a given reference sequence) is calculated as follows:

$$100 \times ( fraction of A / B )$$

where A is the number of amino acid residues scored as identical in the alignment of the candidate sequence and the reference sequence, and where B is the total number of amino acid residues in the reference sequence. In some embodiments where the length of the candidate sequence does not equal to the length of the reference sequence, the percent amino acid sequence identity of the candidate sequence to the reference sequence would not equal to the percent amino acid sequence identity of the reference sequence to the candidate sequence.

Two polynucleotide or polypeptide sequences are said to be “identical” if the sequence of nucleotides or amino acids in the two sequences is the same when aligned for maximum correspondence as described above. Comparisons between two sequences are typically performed by comparing the sequences over a comparison window to identify and compare local regions of sequence similarity. A “comparison window” as used herein, refers to a segment of at least about 15 contiguous positions, about 20 contiguous positions, about 25 contiguous positions, or more (e.g., about 30 to about 75 contiguous positions, or about 40 to about 50 contiguous positions), in which a sequence may be compared to a reference sequence of the same number of contiguous positions after the two sequences are optimally aligned.

The term “treating” or “to treat,” as used herein, refers to a therapeutic treatment of a disease (e.g., cancer, fibrosis, or an infection) in a subject. In some embodiments, a therapeutic treatment may slow the progression of the disease, improve the subject's outcome, and/or eliminate tumors. In some embodiments, a therapeutic treatment of the disease in a subject may alleviate or ameliorate of one or more symptoms or conditions associated with the disease, diminish the extent of the symptoms, stabilize (i.e., not worsening) the state of the disease, prevent the spread of the disease, and/or delay or slow the progress of the disease, as compare the state and/or the condition of the disease in the absence of the therapeutic treatment.

The term “average value of T,” as used herein, refers to the mean number of monomers of CD73 or dimers of CD73 inhibitors conjugated to an Fc domain monomer or Fc domain within a population of conjugates. In some embodiments, within a population of conjugates, the average number of monomers of CD73 inhibitor or dimers of CD73 inhibitors conjugated to an Fc domain monomer may be from 1 to 20 (e.g., the average value of T is 1 to 2, 1 to 3, 1 to 4, 1 to 5, 5 to 10, 10 to 15, or 15 to 20). In some embodiments, the average value of T is 1, 2, 3, 4, 5, 6, 7, 8, 9, 10, 11, 12, 13, 14, 15, 16, 17, 18, 19, or 20.

The term “subject,” as used herein, can be a human or non-human primate.

The term “therapeutically effective amount,” as used herein, refers to an amount, e.g., pharmaceutical dose, effective in inducing a desired effect in a subject or in treating a subject having a condition or disorder described herein. It is also to be understood herein that a “therapeutically effective amount” may be interpreted as an amount giving a desired therapeutic and/or preventative effect, taken in one or more doses or in any dosage or route, and/or taken alone or in combination with other therapeutic agents. For example, in the context of administering a conjugate described herein that is used for the treatment of a disease described herein, an effective amount of a conjugate is, for example, an amount sufficient to prevent, slow down, or reverse the progression of the disease as compared to the response obtained without administration of the conjugate.

As used herein, the term “pharmaceutical composition” refers to a medicinal or pharmaceutical formulation that contains at least one active ingredient as well as one or more excipients and diluents to enable the active ingredient suitable for the method of administration. The pharmaceutical composition of the present disclosure includes pharmaceutically acceptable components that are compatible with a conjugate described herein.

As used herein, the term “pharmaceutically acceptable carrier” refers to an excipient or diluent in a pharmaceutical composition. For example, a pharmaceutically acceptable carrier may be a vehicle capable of suspending or dissolving the active conjugate. The pharmaceutically acceptable carrier must be compatible with the other ingredients of the formulation and not deleterious to the recipient. In the present disclosure, the pharmaceutically acceptable carrier must provide adequate pharmaceutical stability to a conjugate described herein. The nature of the carrier differs with the mode of administration.

For example, for oral administration, a solid carrier is preferred; for intravenous administration, an aqueous solution carrier (e.g., WFI, and/or a buffered solution) is generally used.

The term “pharmaceutically acceptable salt,” as used herein, represents salts of the conjugates described herein that are, within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of the conjugates described herein or separately by reacting the free base group with a suitable organic acid.

The term “about,” as used herein, indicates a deviation of ±5%. For example, about 10% refers to from 9.5% to 10.5%.

Any values provided in a range of values include both the upper and lower bounds, and any values contained within the upper and lower bounds.

Other features and advantages of the conjugates described herein will be apparent from the following Detailed Description and the claims.

### DESCRIPTION OF THE DRAWINGS

FIG. 1A is a graph showing that Conjugate 133a and Conjugate 133b exhibited single digit nM potency in a cell-free CD73 inhibition assay. AB680 is a known CD73 inhibitor. Int-258 corresponds to the CD73 inhibitor portion of Conjugate 133a and 133b, without the Fc domain. FIG. 1B is a graph showing that Conjugate 172a exhibited single digit nM potency in a cell-free CD73 inhibition assay. AB680 is a known CD73 inhibitor. FIG. 1C is a graph showing the percentage of CD73 inhibition of Oleclumab and mupadolimab in a cell-free CD73 inhibition assay.

FIG. 2 is a graph showing that Conjugate 133a and Conjugate 133b exhibited single digit nM potency in a cell-based CD73 inhibition assay (MDA-MB231 cells, human breast cancer). AB680 is a known CD73 inhibitor. Int-258 corresponds to the CD73 inhibitor portion of Conjugate 133a and 133b, without the Fc domain.

FIG. 3 is a graph showing that Conjugate 172a exhibited sub-nanomolar potency in a cell-based CD73 inhibition assay (MDA-MB231 cells, human breast cancer).

FIG. 4 is a graph showing the PBMC rescue assay of AMP suppressed cells using Conjugate 172a.

FIG. 5 is a series of graphs showing that Conjugate 172a exhibited potent and complete CD73 inhibition similar to small molecule inhibitors, AB680, OP5244 and SHR170008 in human PBMC cells.

FIG. 6 is a series of graphs showing that Conjugate 172a exhibited potent and complete CD73 inhibition similar to small molecule inhibitors, AB680, OP5244 and SHR170008 in 4T1 cancer cells.

FIG. 7 is a series of graphs showing that Conjugate 172a exhibited potent, quantitative binding to B cells (CD3⁻CD19⁺) and CD8⁺ T cells (CD3⁺CD8⁺) expressing CD73 with sub-nanomolar and single-digit nanomolar binding, respectively. SEQ ID NO: 80 (Conjugate 172a lacking the Int) was used as a negative control.

FIG. 8 is a graph showing Conjugate 172a exhibited modest activation of B cells (CD3⁻CD19⁺) quantified by CD69 expression. SEQ ID NO: 80 (Conjugate 172a lacking the Int) was used as a negative control.

FIGS. 9A and 9B are a series of graphs showing that Conjugate 172a binds to MDA-MB-231 cells at low concentrations. FIG. 9A shows that the binding of Conjugate 172a in the presence of AMP was reduced in an AMP-dependent manner, demonstrating that Conjugate 172a is an AMP-competitive CD73 inhibitor. FIG. 9B shows that the binding of Conjugate 172a in the presence of increasing concentrations of a small molecule CD73 inhibitor, AB680, was reduced in a dose-dependent manner, demonstrating that Conjugate 172a is a catalytic site CD73 inhibitor.

FIGS. 10A and 10B are a series of graphs showing that Conjugate 133b reactivates T-cells suppressed by adenosine via inhibition of CD73. Conjugate 133b had an EC₅₀ of 175.3 nM in the presence of 30 μM adenosine monophosphate (AMP) (FIG. 10A) or an EC₅₀ of 976 nM in the presence of 100 μM AMP (FIG. 10B).

FIG. 11 is a graph showing that Conjugate 172a and the anti-CD73 monoclonal antibodies exhibited potent sub-nanomolar CD73 internalization activity in MDA-MB-231 cells.

FIG. 12 is a graph showing that Conjugate 172a exhibits three-fold stronger binding to MDA-MB-231 cells (IC₅₀ 0.39 nM) than Mupdolimab (IC₅₀ 1.25 nM). SEQ ID NO: 80 (the hlgG1 Fc carrier) was used as a negative control.

FIG. 13 is a graph showing a 7-day mouse pharmacokinetic study for Conjugate 133a at 10 mg/kg administered intramuscularly. After 168 h, similar plasma exposure levels were observed for the CD73 (28.6 μg/mL) and Fc (21.5 μg/mL) capture assays. The AUCs for the CD73 (3565) and Fc (7538) captures were within approximately 2-fold of each other, suggesting minimal loss of the Int overtime.

FIG. 14 is a graph showing a 7-day mouse pharmacokinetic study for Conjugate 172a at 10 mg/kg administered intramuscularly. After 168 h, similar plasma exposure levels were observed for the CD73 and Fc capture assays. These results highlight the long half-life, high plasma exposures and overall stability of Conjugate 172a in vivo.

FIG. 15 is a graph showing the efficacy of Conjugate 133a in a mouse syngeneic model with a colon tumor cell line.

FIG. 16 is a graph showing the efficacy of Conjugate 133b in a mouse syngeneic model with a colon tumor cell line. Conjugate 133b was administered alone (5 mg/kg or 20 mg/kg) or in combination with an anti-PD-1 antibody (RMP1-14).

FIG. 17 is a graph showing the efficacy overtime (12 days) of Conjugate 133b in a mouse syngeneic model with a colon tumor cell line. Conjugate 133b was administered alone (5 mg/kg or 20 mg/kg) or in combination with an anti-PD-1 antibody (RMP1-14).

FIG. 18 is a graph showing the efficacy of Conjugate 133b and Conjugate 161 against CT26 (colon) tumors in mice after 10 days of growth.

FIGS. 19A and 19B are a series of graphs showing the efficacy of Conjugate 133b, Conjugate 161, Conjugate 169, Conjugate 165, Conjugate 172a, and Conjugate 175 against a colon tumor cell line (CT26) in a syngeneic mouse model.

FIG. 20A is a graph showing tumor growth over 8 days of a vehicle control vs. animals treated with Conjugate 172a. FIG. 20B is a bar chart showing percentage of tumor growth inhibition by Conjugate 172a relative to vehicle control in animals. FIG. 20C is a box plot showing tumor volumes for individual animals on Day 8 of the study.

FIG. 21 is a graph of Conjugate 172a plasma levels overtime in mice.

FIGS. 22A and 22B are a series of graphs showing the effect of different dosing schedules with respect to and Conjugate 133b efficacy against CT26 (colon) tumor growth in mice.

FIG. 23 is a graph showing the IV tolerability of Conjugate 172a in mice.

FIG. 24 is a graph showing that Conjugate 172a and Conjugate 172c exhibited nM potency in a cell-free CD73 inhibition assay.

FIG. 25 is a graph showing that various batches of Conjugate 172c exhibited either single digit nM or sub nM potency in a cell-based CD73 inhibition assay (MDA-MB231 cells, human breast cancer).

FIG. 26 is a series of graphs showing the binding of Conjugate 172c (batch 9), Oleclumab, and Mupadolimab in the presence and absence of AMP to human MDA-MB-231 cancer cells, determined by flow cytometry.

FIG. 27 is a series of graphs showing the binding of Conjugate 172c (batch 9), Oleclumab, and Mupadolimab in the presence and absence of small molecule CD73 inhibitor, AB680, to human MDA-MB-231 cancer cells, determined by flow cytometry.

FIG. 28 is a graph showing the activity of Conjugate 172c (batch 9) and comparators targeting CD73 in a CD73 inhibition assay using human PBMCs at 3 h.

FIG. 29 is a graph showing the activity of Conjugate 172c (batch 9) and comparators targeting CD73 in a CD73 inhibition assay using human PBMCs at 24 h.

FIG. 30 is a graph showing the activity of Conjugate 172c (batch 9) and comparators targeting CD73 in a CD73 inhibition assay using mouse EMT6 cancer cells at 3 h,

FIG. 31 is a graph showing the activity of Conjugate 172c (batch 9) and comparators targeting CD73 in a CD73 inhibition assay using mouse EMT6 cancer cells at 24 h.

FIG. 32 is a series of graphs showing the activity of Conjugate 172c (batch 9) and other test articles targeting CD73 in a human PBMC activation assay by flow cytometry.

FIG. 33 is a graph showing the activity of Conjugate 172c (batch 9) and other test articles targeting CD73 in a human PBMC activation assay measuring percent inhibition of adenosine production by CellTiter-Glo.

FIG. 34 is a graph showing the percent of CD73 internalization dose response curves of Conjugate 172c (batch 9), Oleclumab, Mupadolimab, and hlgG Fc into MDA-MB-231 human breast adenocarcinoma cells.

FIG. 35 is a graph showing the 14-day plasma concentration-time curves by CD73 capture/Fc detection following administration of the dose linearity concentrations of Conjugate 172a.

FIG. 36 is a graph showing the 14-day plasma concentration-time curves by Fc capture/Fc detection following administration of the dose linearity concentrations of Conjugate 172a.

FIG. 37 is a graph showing the 14-day plasma concentration-time curves by CD73 capture/Fc detection following administration of the dose linearity concentrations of Conjugate 172a.

FIG. 38 is a graph showing the 14-day plasma concentration-time curves by Fc capture/Fc detection following administration of the dose linearity concentrations of Conjugate 172a.

FIG. 39 is a graph showing the 7-day plasma concentration-time curves following the IP administration of the Conjugate 172c DAR scan by CD73 capture/Fc detect.

FIG. 40 is a graph showing the 7-day plasma concentration-time curves following the IP administration of the Conjugate 172c DAR scan by Fc capture/Fc detect.

FIG. 41 is a graph showing the 14-day plasma concentration-time curves by CD73 capture/Fc detection following IV, IP and SC dosing of Conjugate 172a.

FIG. 42 is a graph showing the 14-day plasma concentration-time curves by Fc capture/Fc detection following IV, IP and SC dosing of Conjugate 172a.

FIG. 43 is a bar chart showing the diameter of 3D tumor spheroids in the presence of Conjugate 201 and other test articles at 100 nM.

FIG. 44 is a bar chart showing the penetration of 3D tumor spheroids in microns in the presence of 100 nM Conjugate 201 and 100 nM Oleclumab.

FIG. 45A is a graph showing the average tumor volumes (±SEM) in mice treated with Conjugate 172a, Conjugate 172a/α-PD-1, and other test articles as a function of time. FIG. 45B is a graph showing the average tumor volumes in mice treated with Conjugate 172a, Conjugate 172a/α-PD-1, and other test articles on Day 22 (Day 17 post-dose).

FIG. 46A is a graph showing the average tumor volumes (±SEM) in mice treated with Conjugate 172a, Conjugate 172a/α-PD-1, α-PD-1, and vehicle as a function of time. FIG. 46B is a graph showing the tumor growth over time in mice with fully regressed tumors that were treated with Conjugate 172a/α-PD-1.

FIG. 47 is a timeline of the re-challenge study in mice to determine if fully regressed animals treated with the Conjugate 172a/anti-PD-1 combination also acquired immunity to the EMT-6 cancer cell line.

FIG. 48A is a box plot showing tumor inhibition in mice treated with either Conjugate 172a or vehicle against the EMT-6 breast cancer cell line. FIG. 48B is a line graph showing tumor inhibition in mice treated with either Conjugate 172a or vehicle against the EMT-6 breast cancer cell line.

FIG. 49 is a graph showing the activity of conjugates with different linker lengths targeting CD73 in a PBMC activation assay using CD25⁺ of CD8⁺ T cells as a read out.

FIG. 50A is a graph showing the inhibition of purified recombinant human CD73 in the presence of Conjugate 172c (batch 9) or CD73 small molecule inhibitors using a cell-free CD73 enzyme inhibition assay. FIG. 50B is a graph showing the inhibition of purified recombinant human CD73 in the presence of Conjugate 172c (batch 9) or CD73 monoclonal antibodies using a cell-free CD73 enzyme inhibition assay FIG. 51 is a graph showing the inhibition of surface-expressed CD73 on MDA-MB231 (human breast cancer) cells in the presence of Conjugate 172c (batch 9) and other test articles using a cell-based CD73 enzyme inhibition assay.

FIG. 52 is a graph showing the inhibition of surface-expressed CD73 on MDA-MB231 (human breast cancer) cells in the presence of various conjugates containing different linker lengths using a cell-based CD73 enzyme inhibition assay.

FIG. 53A is a graph showing the tumor volume in a mouse colon tumor model overtime treated with Conjugate 172c (batch 9), and in combination with an α-PD-1 mAb. FIG. 53B is a graph showing the tumor volume of individual mice treated with Conjugate 172c (batch 9), and in combination with an α-PD-1 mAb, on day 20.

### DETAILED DESCRIPTION

This disclosure relates to conjugates including an Fc domain monomer or Fc domain covalently linked to a moiety that binds to or inhibits CD73. In particular, such conjugates contain monomers or dimers of a moiety that binds to or inhibits CD73 conjugated to an Fc monomer or Fc domain. The CD73 inhibitor (e.g., adenosine monophosphate, adenosine bisphosphate, or an analog thereof) in the conjugate targets CD73. The Fc monomers or Fc domains in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC). The featured conjugates exhibit desirable tissue distribution (e.g., lung distribution).

This disclosure also provides pharmaceutical compositions including such conjugates and uses of such conjugates in the treatment of disorders associated with dysregulation or overexpression of CD73 (e.g., cancer, fibrosis, or a viral infection).

### I. Conjugates

Provided herein are synthetic conjugates that include an Fc domain conjugated to one or more CD73 inhibitors (e.g., a CD73 inhibitor of formula (A), (A-I), (A-Ia), (A-Ib), (A-Ib-1), (A-Ib-2), (A-II), (A-IIa), (A-IIb), (A-IIb-1), or (A-IIb-2)) or one or more dimers of two CD73 inhibitors. The dimers of two CD73 inhibitors include a CD73 inhibitor (e.g., a first CD73 inhibitor of formula (A), (A-I), (A-Ia), (A-Ib), (A-Ib-1), (A-Ib-2), (A-II), (A-IIa), (A-IIb), (A-IIb-1), or (A-IIb-2)) and a second CD73 inhibitor (e.g., a second CD73 inhibitor of formula (A), (A-I), (A-Ia), (A-Ib), (A-Ib-1), (A-Ib-2), (A-II), (A-IIa), (A-IIb), (A-IIb-1), or (A-IIb-2)). In the context of dimers, e.g., of formula (D-I), the first and second CD73 inhibitors are linked to each other by way of a linker.

Conjugates of the disclosure include CD73 inhibitor monomers and dimers conjugated to an Fc domain, Fc monomer, or Fc-binding peptide. The Fc domain in the conjugates described herein binds to the FcγRs (e.g., FcRn, FcγRI, FcγRIIa, FcγRIIc, FcγRIIIa, and FcγRIIIb) on immune cells. The binding of the Fc domain in the conjugates described herein to the FcγRs on immune cells activates phagocytosis and effector functions, such as antibody-dependent cell-mediated cytotoxicity (ADCC).

Conjugates provided herein are described by any one of formulas (D-I) or (M-I). In some embodiments, the conjugates described herein include one or more monomers of CD73 inhibitors conjugated to an Fc domain monomer or Fc domain. In some embodiments, the conjugates described herein include one or more dimers of CD73 inhibitors conjugated to an Fc domain monomer or Fc domain. In some embodiments, when n is 2, E (an Fc domain monomer) dimerizes to form an Fc domain.

Conjugates described herein may be synthesized using available chemical synthesis techniques in the art. In cases where a functional group is not available for conjugation, a molecule may be derivatized using conventional chemical synthesis techniques that are well known in the art. In some embodiments, the conjugates described herein contain one or more chiral centers. The conjugates include each of the isolated stereoisomeric forms as well as mixtures of stereoisomers in varying degrees of chiral purity, including racemic mixtures. It also encompasses the various diastereomers, enantiomers, and tautomers that can be formed.

### Conjugates of Monomers of CD73 Inhibitors Linked to an Fc Domain

In some embodiments, the conjugates described herein include an Fc domain monomer or Fc domain covalently linked to one or more monomers of CD73 inhibitors, e.g., a conjugate described by formula (M-I). Conjugates of an Fc domain monomer or Fc domain and one or more monomers of CD73 inhibitors may be formed by linking the Fc domain monomer or Fc domain to each of the monomers of CD73 inhibitors through a linker, such as any of the linkers described herein.

In the conjugates having an Fc domain monomer or Fc domain covalently linked to one or more monomers of CD73 inhibitors described herein, the squiggly line connected to E indicates that one or more (e.g., 1, 2, 3, 4, 5, 6, 7, 8, 9, 10, 11, 12, 13, 14, 15, 16, 17, 18, 19, or 20) monomers of CD73 inhibitors may be attached to an Fc domain monomer or Fc domain. In some embodiments, when n is 1, one or more (e.g., 1, 2, 3, 4, 5, 6, 7, 8, 9, or 10) monomers of CD73 inhibitors may be attached to an Fc domain monomer. In some embodiments, when n is 2, one or more (e.g., 1, 2, 3, 4, 5, 6, 7, 8, 9, 10, 11, 12, 13, 14, 15, 16, 17, 18, 19, or 20) monomers of CD73 inhibitors may be attached to an Fc domain. The squiggly line in the conjugates described herein is not to be construed as a single bond between one or more monomers of CD73 inhibitors and an atom in the Fc domain monomer or Fc domain. In some embodiments, when T is 1, one monomer of CD73 inhibitor may be attached to an atom in the Fc domain monomer or Fc domain. In some embodiments, when T is 2, two monomers of CD73 inhibitors may be attached to an atom in the Fc domain monomer or Fc domain.

In some embodiments, when T is greater than 1 (e.g., T is 2, 3, 4, 5, 6, 7, 8, 9, 10, 11, 12, 13, 14, 15, 16, 17, 18, 19, or 20), each A₁-L may be independently selected (e.g., independently selected from any of the A₁-L structures described herein). In some embodiments, E may be conjugated to 2, 3, 4, 5, 6, 7, 8, 9, 10, or more different A₁-L moieties. In some embodiments, E is conjugated to a first A₁-L moiety, and a second A₁-L, moiety. In some embodiments, A₁ of each of the first A₁-L moiety and the second A₁-L moiety is independently selected from any one of formulas (A), (A-I), (A-Ia), (A-Ib), (A-Ib-1), (A-Ib-2), (A-II), (A-IIa), (A-IIb), (A-IIb-1), and (A-IIb-2).

In some embodiments, the first A₁-L moiety is conjugated specifically to lysine residues of E (e.g., the nitrogen atoms of surface exposed lysine residues of E), and the second A₁-L moiety is conjugated specifically to cysteine residues of E (e.g., the sulfur atoms of surface exposed cysteine residues of E). In some embodiments, the first A₁-L moiety is conjugated specifically to cysteine residues of E (e.g., the sulfur atoms of surface exposed cysteine residues of E), and the second A₁-L moiety is conjugated specifically to lysine residues of E (e.g., the nitrogen atoms of surface exposed lysine residues of E).

As described further herein, a linker in a conjugate having an Fc domain monomer or Fc domain covalently linked to one or more monomers of the CD73 inhibitors described herein (e.g., L) may be a divalent structure having two arms. One arm in a divalent linker may be attached to the monomer of the CD73 inhibitor and the other arm may be attached to the Fc domain monomer or Fc domain.

In conjugates having an Fc domain covalently linked to one or more monomers of CD73 inhibitors, as represented by the formulae above, when n is 2, two Fc domain monomers (each Fc domain monomer is represented by E) dimerize to form an Fc domain.

### Conjugates of Dimers of CD73 Inhibitors Linked to an Fc Domain

The conjugates described herein include an Fc domain monomer or Fc domain covalently linked to one or more dimers of CD73 inhibitors, e.g., a conjugate described by formula (D-I). The dimers of two CD73 inhibitors include a first CD73 inhibitor (e.g., a first CD73 inhibitor of formula (A), (A-I), (A-Ia), (A-Ib), (A-Ib-1), (A-Ib-2), (A-II), (A-IIa), (A-IIb), (A-IIb-1), or (A-IIb-2)) and a second CD73 inhibitor (e.g., a second CD73 inhibitor of formula (A), (A-I), (A-Ia), (A-Ib), (A-Ib-1), (A-Ib-2), (A-II), (A-IIa), (A-IIb), (A-IIb-1), or (A-IIb-2)). The first and second CD73 inhibitors are linked to each other by way of a linker, such as a linker described herein. In some embodiments of the dimers of CD73 inhibitors, the first and second CD73 inhibitors are the same. In some embodiments, the first and second CD73 inhibitors are different.

In some embodiments, when T is greater than 1 (e.g., T is 2, 3, 4, 5, 6, 7, 8, 9, 10, 11, 12, 13, 14, 15, 16, 17, 18, 19, or 20), each A₁-L-A₂ may be independently selected (e.g., independently selected from any of the A₁-L-A₂ structures described herein). In some embodiments, E may be conjugated to 2, 3, 4, 5, 6, 7, 8, 9, 10, or more different A₁-L-A₂ moieties. In some embodiments, E is conjugated to a first A₁-L-A₂ moiety, and a second A₁-L-A₂, moiety. In some embodiments, each of A₁ and A₂ of the first A₁-L-A₂ moiety and of the second A₁-L-A₂ moiety are independently selected from any one of formulas (A), (A-I), (A-Ia), (A-Ib), (A-Ib-1), (A-Ib-2), (A-II), (A-IIa), (A-IIb), (A-IIb-1), or (A-IIb-2).

In some embodiments, the first A₁-L-A₂ moiety is conjugated specifically to lysine residues of E (e.g., the nitrogen atoms of surface exposed lysine residues of E), and the second A₁-L-A₂ moiety is conjugated specifically to cysteine residues of E (e.g., the sulfur atoms of surface exposed cysteine residues of E). In some embodiments, the first A₁-L-A₂ moiety is conjugated specifically to cysteine residues of E (e.g., the sulfur atoms of surface exposed cysteine residues of E), and the second A₁-L-A₂ moiety is conjugated specifically to lysine residues of E (e.g., the nitrogen atoms of surface exposed lysine residues of E).

In the conjugates described herein, the squiggly line connected to E indicates that one or more (e.g., 1, 2, 3, 4, 5, 6, 7, 8, 9, 10, 11, 12, 13, 14, 15, 16, 17, 18, 19, or 20) dimers of CD73 inhibitors may be attached to an Fc domain monomer or Fc domain. In some embodiments, when n is 1, one or more (e.g., 1, 2, 3, 4, 5, 6, 7, 8, 9, or 10) dimers of CD73 inhibitors may be attached to an Fc domain monomer.

In some embodiments, when n is 2, one or more (e.g., 1, 2, 3, 4, 5, 6, 7, 8, 9, 10, 11, 12, 13, 14, 15, 16, 17, 18, 19, or 20) dimers of CD73 inhibitors may be attached to an Fc domain. The squiggly line in the conjugates described herein is not to be construed as a single bond between one or more dimers of CD73 inhibitors and an atom in the Fc domain monomer or Fc domain. In some embodiments, when T is 1, one dimer of CD73 inhibitors may be attached to an atom in the Fc domain monomer or Fc domain. In some embodiments, when T is 2, two dimers of CD73 inhibitors may be attached to an atom in the Fc domain monomer or Fc domain.

As described further herein, a linker in a conjugate described herein may be a branched structure. As described further herein, a linker in a conjugate described herein may be a multivalent structure, e.g., a divalent or trivalent structure having two or three arms, respectively. In some embodiments when the linker has three arms, two of the arms may be attached to the first and second CD73 inhibitors and the third arm may be attached to the Fc domain monomer or Fc domain.

In conjugates having an Fc domain covalently linked to one or more dimers of CD73 inhibitors, as represented by the formulae above, when n is 2, two Fc domain monomers (each Fc domain monomer is represented by E) dimerize to form an Fc domain.

### II. Fc Domain Monomers and Fc Domains

The disclosure features compositions (e.g., conjugates) which include one or more Fc domain monomers. When two compositions including an Fc domain monomer dimerize, the resulting conjugate includes an Fc domain. An Fc domain monomer includes a hinge domain, a C_{H2} antibody constant domain, and a C_{H}3 antibody constant domain. The Fc domain monomer can be of immunoglobulin antibody isotype IgG, IgE, IgM, IgA, or IgD. The Fc domain monomer can also be of any immunoglobulin antibody isotype (e.g., IgG1, IgG2a, IgG2b, IgG3, or IgG4). The Fc domain monomer can be of any immunoglobulin antibody allotype (e.g., IGHG1*01 (i.e., G1m(za)), IGHG1*07 (i.e., G1m(zax)), IGHG1*04 (i.e., G1m(zav)), IGHG1*03 (G1m(f)), IGHG1*08 (i.e., G1m(fa)), IGHG2*01, IGHG2*06, IGHG2*02, IGHG3*01, IGHG3*05, IGHG3*10, IGHG3*04, IGHG3*09, IGHG3*11, IGHG3*12, IGHG3*06, IGHG3*07, IGHG3*08, IGHG3*13, IGHG3*03, IGHG3*14, IGHG3*15, IGHG3*16, IGHG3*17, IGHG3*18, IGHG3*19, IGHG2*04, IGHG4*01, IGHG4*03, or IGHG4*02) (as described in, for example, Vidarsson et al. IgG subclasses and allotypes: from structure to effector function. Frontiers in Immunology. 5(520):1-17 (2014)). The Fc domain monomer can also be of any species, e.g., human, murine, or mouse. A dimer of Fc domain monomers is an Fc domain that can bind to an Fc receptor, which is a receptor located on the surface of leukocytes.

In some embodiments, an Fc domain monomer described herein may contain one or more amino acid substitutions, additions, and/or deletion relative to an Fc domain monomer having a sequence of any one of SEQ ID NOs: 1-112 and 115-120. In some embodiments, an Asn (e.g., N297) in an Fc domain monomer in the conjugates as described herein may be replaced by Ala (e.g., N297A), Gly (e.g., N297G), or Gln (e.g., N297Q) in order to prevent N-linked glycosylation. In some embodiments, the amino acid corresponding to N297 is substituted with Ala, Gly, or Gln.

In some embodiments, an Fc domain monomer in a conjugate described herein includes an additional moiety for purification (e.g., a hexa-histidine peptide), or a signal sequence (e.g., IL2 signal sequence) attached to the N- or C-terminus of the Fc domain monomer. In some embodiments, an additional moiety for purification (e.g., a hexa-histidine peptide), or a signal sequence (e.g., IL2 signal sequence) attached to the N- or C-terminus of the Fc domain monomer is cleaved after expression of the polypeptide. In some embodiments, an Fc domain monomer in the compositions does not contain any type of antibody variable region, e.g., V_{H}, V_{L}, a complementarity determining region (CDR), or a hypervariable region (HVR).

In some embodiments, an Fc domain monomer in a conjugate described herein may have a sequence that is at least 95% identical (e.g., 97%, 99%, or 99.5% identical) to the sequence of any one of SEQ ID NOs: 1-112 and 115-120, shown below. In some embodiments, an Fc domain monomer in the fusion proteins or conjugates as described herein may include a sequence of any one of SEQ ID NOs: 1-112 and 115-120, shown below.

SEQ ID NO: 1: mature human Fc IgG1, Z₁ is Cys or Ser, and wherein X₁ is Met or Tyr, X₂ is Ser
or Thr, X₃ is Thr or Glu, X₄ is Asp or Glu, and X₅ is Leu or Met, X₆ is Met or Leu, and X₇ is
Asn or Ser
NVNHKPSNTKVDKKVEPKSZ₁DKTHTCPPCPAPELLGGPSVFLFPPKPKDTLX₁IX₂RX₃PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX5TKNQVSLTCLVKGFYPSDIAVEWESNGQPENNYKTTPPVLDSDGSFF
LYSKLTVDKSRWQQGNVFSCSVX₆HEALHX₇HYTQKSLSLSPGK
SEQ ID NO: 2: mature human Fc IgG1, Cys to Ser substitution (#), and wherein X₁ is Met or
Tyr, X₂ is Ser or Thr, X₃ is Thr or Glu, X₄ is Asp or Glu, and X₅ is Leu or Met, X₆ is Met or
Leu, and X₇ is Asn or Ser
NVNHKPSNTKVDKKVEPKSS(#)DKTHTCPPCPAPELLGGPSVFLFPPKPKDTLX₁IX₂RX₃PEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSRX₄EX₅TKNQVSLTCLVKGFYPSDIAVEWESNGQPENNYKTTPPVLDSDGSF
FLYSKLTVDKSRWQQGNVFSCSVX₆HEALHX₇HYTQKSLSLSPGK
SEQ ID NO: 3: mature human IgG1 Fc, Cys to Ser substitution (#), X₄ is Asp or Glu, and X₅ is
Leu or Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 4: mature human IgG1 Fc, Cys to Ser substitution (#), allotype G1m(f) (bold
italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 5: mature human IgG1 Fc, Cys to Ser substitution (#), allotype G1m(fa) (bold
italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 6: mature human IgG1 Fc, Cys to Ser substitution (#), M428L, N434S mutations
(Bold/Underlined), allotype G1m(fa) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVLHEALHSHYTQKSLSLSPGK
SEQ ID NO: 7: mature human IgG1 Fc, Cys to Ser substitution (#), M428L, N434S mutations
(Bold/Underlined), allotype G1m(f) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVLHEALHSHYTQKSLSLSPGK
SEQ ID NO: 8: mature human IgG1 Fc, Cys to Ser substitution (#), YTE triple mutation (bold
and underlined), allotype G1m(fa) (bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 9: mature human IgG1 Fc, Cys to Ser substitution (#), YTE triple mutation (bold
and underlined), allotype G1m(f) (bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 10: mature human Fc IgG1, Z₁ is Cys or Ser, and wherein X₁ is Met or Tyr, X₂ is
Ser or Thr, X₃ is Thr or Glu, X₄ is Asp or Glu, and X₅ is Leu or Met, X₆ is Met or Leu, and X₇
is Asn or Ser
NVNHKPSNTKVDKKVEPKSZ₁DKTHTCPPCPAPELLGGPSVFLFPPKPKDTLX₁IX₂RX₃PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVX₆HEALHX₇HYTQKSLSLSPG
SEQ ID NO: 11: mature human Fc IgG1, Cys to Ser substitution (#), and wherein X₁ is Met or
Tyr, X₂ is Ser or Thr, X₃ is Thr or Glu, X₄ is Asp or Glu, and X₅ is Leu or Met, X₆ is Met or
Leu, and X₇ is Asn or Ser
NVNHKPSNTKVDKKVEPKSS(#)DKTHTCPPCPAPELLGGPSVFLFPPKPKDTLX₁IX₂RX₃PEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSRX₄EX₅TKNQVSLTCLVKGFYPSDIAVEWESNGQPENNYKTTPPVLDSDGSF
FLYSKLTVDKSRWQQGNVFSCSVX₆HEALHX₇HYTQKSLSLSPG
SEQ ID NO: 12: mature human IgG1 Fc, Cys to Ser substitution (#), X₄ is Asp or Glu, and X₅
is Leu or Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 13: mature human IgG1 Fc, Cys to Ser substitution (#), allotype G1m(f) (bold
italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 14: mature human IgG1 Fc, Cys to Ser substitution (#), allotype G1m(fa) (bold
italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 15: mature human IgG1 Fc, Cys to Ser substitution (#), M428L, N434S mutations
(Bold/Underlined), allotype G1m(fa) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVLHEALHSHYTQKSLSLSPG
SEQ ID NO: 16: mature human IgG1 Fc, Cys to Ser substitution (#), M428L, N434S mutations
(Bold/Underlined), allotype G1m(f) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVLHEALHSHYTQKSLSLSPG
SEQ ID NO: 17: mature human IgG1 Fc, Cys to Ser substitution (#), YTE triple mutation (bold
and underlined), allotype G1m(fa) (bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 18: mature human IgG1 Fc, Cys to Ser substitution (#), YTE triple mutation (bold
and underlined), allotype G1m(f) (bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 19: mature human Fc IgG1, Z₁ is Cys or Ser, and wherein X₁ is Met or Tyr, X₂ is
Ser or Thr, X₃ is Thr or Glu, X₄ is Asp or Glu, and X₅ is Leu or Met, X₆ is Met or Leu, and X₇
is Asn or Ser
VNHKPSNTKVDKKVEPKSZ₁DKTHTCPPCPAPELLGGPSVFLFPPKPKDTLX₁IX₂RX₃PEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRX₄EX₅TKNQVSLTCLVKGFYPSDIAVEWESNGQPENNYKTTPPVLDSDGSFFL
YSKLTVDKSRWQQGNVFSCSVX₆HEALHX₇HYTQKSLSLSPGK
SEQ ID NO: 20: mature human Fc IgG1, Cys to Ser substitution (#), and wherein X₁ is Met or
Tyr, X₂ is Ser or Thr, X₃ is Thr or Glu, X₄ is Asp or Glu, and X₅ is Leu or Met, X₆ is Met or
Leu, and X₇ is Asn or Ser
VNHKPSNTKVDKKVEPKSS(#)DKTHTCPPCPAPELLGGPSVFLFPPKPKDTLX₁IX₂RX₃PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVX₆HEALHX₇HYTQKSLSLSPGK
SEQ ID NO: 21: mature human IgG1 Fc, Cys to Ser substitution (#), X₄ is Asp or Glu, and X₅
is Leu or Met
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRX₄EX₅TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 22: mature human IgG1 Fc, Cys to Ser substitution (#), allotype G1m(f) (bold
italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 23: mature human IgG1 Fc, Cys to Ser substitution (#), allotype G1m(fa) (bold
italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 24: mature human IgG1 Fc, Cys to Ser substitution (#), M428L, N434S mutations
(Bold/Underlined), allotype G1m(fa) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVLHEALHSHYTQKSLSLSPGK
SEQ ID NO: 25: mature human IgG1 Fc, Cys to Ser substitution (#), M428L, N434S mutations
(Bold/Underlined), allotype G1m(f) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVLHEALHSHYTQKSLSLSPGK
SEQ ID NO: 26: mature human IgG1 Fc, Cys to Ser substitution (#), YTE triple mutation (bold
and underlined), allotype G1m(fa) (bold italics)
VNHKPSNTKVDKKVEPKSS(#)DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 27: mature human IgG1 Fc, Cys to Ser substitution (#), YTE triple mutation (bold
and underlined), allotype G1m(f) (bold italics)
VNHKPSNTKVDKKVEPKSS(#)DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
SEQ ID NO: 28: mature human Fc IgG1, Z₁ is Cys or Ser, and wherein X₁ is Met or Tyr, X₂ is
Ser or Thr, X₃ is Thr or Glu, X₄ is Asp or Glu, and X₅ is Leu or Met, X₆ is Met or Leu, and
X₇ is Asn or Ser
VNHKPSNTKVDKKVEPKSZ₁DKTHTCPPCPAPELLGGPSVFLFPPKPKDTLX₁IX₂RX₃PEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRX₄EX₅TKNQVSLTCLVKGFYPSDIAVEWESNGQPENNYKTTPPVLDSDGSFFL
YSKLTVDKSRWQQGNVFSCSVX₆HEALHX₇HYTQKSLSLSPG
SEQ ID NO: 29: mature human Fc IgG1, Cys to Ser substitution (#), and wherein X₁ is Met or
Tyr, X₂ is Ser or Thr, X₃ is Thr or Glu, X₄ is Asp or Glu, and X₅ is Leu or Met, X₆ is Met or
Leu, and X₇ is Asn or Ser
VNHKPSNTKVDKKVEPKSS(#)DKTHTCPPCPAPELLGGPSVFLFPPKPKDTLX₁IX₂RX₃PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVX₆HEALHX₇HYTQKSLSLSPG
SEQ ID NO: 30: mature human IgG1 Fc, Cys to Ser substitution (#), X₄ is Asp or Glu, and X₅
is Leu or Met
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRX₄EX₅TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 31: mature human IgG1 Fc, Cys to Ser substitution (#), allotype G1m(f) (bold
italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 32: mature human IgG1 Fc, Cys to Ser substitution (#), allotype G1m(fa) (bold
italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 33: mature human IgG1 Fc, Cys to Ser substitution (#), M428L, N434S mutations
(Bold/Underlined), allotype G1m(fa) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVLHEALHSHYTQKSLSLSPG
SEQ ID NO: 34: mature human IgG1 Fc, Cys to Ser substitution (#), M428L, N434S mutations
(Bold/Underlined), allotype G1m(f) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVLHEALHSHYTQKSLSLSPG
SEQ ID NO: 35: mature human IgG1 Fc, Cys to Ser substitution (#), YTE triple mutation (bold
and underlined), allotype G1m(fa) (bold italics)
VNHKPSNTKVDKKVEPKSS(#)DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 36: mature human IgG1 Fc, Cys to Ser substitution (#), YTE triple mutation (bold
and underlined), allotype G1m(f) (bold italics)
VNHKPSNTKVDKKVEPKSS(#)DKTHTCPPCPAPELLGGPSVFLFPPKPKDTLYITREPEVTCVVVDVSHE
DPEVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPG
SEQ ID NO: 37: mature human Fc IgG1, J₁ is Asn or absent, J₂ is Lys or absent, Z₁ is Cys or
Ser, and wherein X₁ is Met or Tyr, X₂ is Ser or Thr, X₃ is Thr or Glu, X₄ is Asn or Ala, X₅
is Leu or Asp, X₆ is Gln or His, X₇ is Asp or Glu, and X₈ is Leu or Met, X₉ is Met or Leu, and
X₁₀ is Asn or Ser
J₁VNHKPSNTKVDKKVEPKSZ₁DKTHTCPPCPAPELLGGPSVFLFPPKPKDTLX₁IX₂RX₃PEVTCVVVDVSH
EDPEVKFNWYVDGVEVHNAKTKPREEQYX₄STYRVVSVLTVX₅HX₆DWLNGKEYKCKVSNKALPAPIEKTI
SKAKGQPREPQVYTLPPSRX₇EX₈TKNQVSLTCLVKGFYPSDIAVEWESNGQPENNYKTTPPVLDSDGSF
FLYSKLTVDKSRWQQGNVFSCSVX₉HEALHX₁₀HYTQKSLSLSPGJ₂
SEQ ID NO: 38: mature human Fc IgG1, Cys to Ser substitution (#), J₁ is Asn or absent, J₂ is
Lys or absent, and wherein X₄ is Asn or Ala, X₅ is Leu or Asp, X₆ is Gln or His, X₇ is Asp or
Glu, and X₈ is Leu or Met, and X₁₀ is Asn or Ser
J₁VNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYX₄STYRVVSVLTVX₅HX₆DWLNGKEYKCKVSNKALPAPIEKTI
SKAKGQPREPQVYTLPPSRX₇EX₈TKNQVSLTCLVKGFYPSDIAVEWESNGQPENNYKTTPPVLDSDGSF
FLYSKLTVDKSRWQQGNVFSCSVMHEALHX₁₀HYTQKSLSLSPGJ₂
SEQ ID NO: 39: mature human Fc IgG1, Cys to Ser substitution (#), DHS triple mutation (bold
and underlined), J₁ is Asn or absent, J₂ is Lys or absent, wherein X₄ is Asn or Ala, X₇ is
Asp or Glu, and X₈ is Leu or Met
J₁VNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYX₄STYRVVSVLTVDHHDWLNGKEYKCKVSNKALPAPIEKTIS
KAKGQPREPQVYTLPPSRX₇EX₈TKNQVSLTCLVKGFYPSDIAVEWESNGQPENNYKTTPPVLDSDGSFF
LYSKLTVDKSRWQQGNVFSCSVMHEALHSHYTQKSLSLSPGJ₂
SEQ ID NO: 40: mature human Fc IgG1, Cys to Ser substitution (#), DHS triple mutation (bold
and underlined), wherein X₄ is Asn or Ala, X₇ is Asp or Glu, and X₈ is Leu or Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYX₄STYRVVSVLTVDHHDWLNGKEYKCKVSNKALPAPIEKTIS
KAKGQPREPQVYTLPPSRX₇EX₈TKNQVSLTCLVKGFYPSDIAVEWESNGQPENNYKTTPPVLDSDGSFF
LYSKLTVDKSRWQQGNVFSCSVMHEALHSHYTQKSLSLSPGK
SEQ ID NO: 41: mature human Fc IgG1, Cys to Ser substitution (#), DHS triple mutation (bold
and underlined), wherein X₇ is Asp or Glu and X₈ is Leu or Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEKTIS
KAKGQPREPQVYTLPPSRX₇EX₈TKNQVSLTCLVKGFYPSDIAVEWESNGQPENNYKTTPPVLDSDGSFF
LYSKLTVDKSRWQQGNVFSCSVMHEALHSHYTQKSLSLSPGK
SEQ ID NO: 42: mature human Fc IgG1, Cys to Ser substitution (#), DHS triple mutation (bold
and underlined), allotype G1m(fa) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHSHYTQKSLSLSPGK
SEQ ID NO: 43: mature human Fc IgG1, Cys to Ser substitution (#), DHS triple mutation (bold
and underlined), allotype G1m(f) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHSHYTQKSLSLSPGK
SEQ ID NO: 44: mature human Fc IgG1, Cys to Ser substitution (#), DHS triple mutation (bold
and underlined), allotype G1m(fa) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVDHHDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHSHYTQKSLSLSPGK
SEQ ID NO: 45: mature human Fc IgG1, Cys to Ser substitution (#), DHS triple mutation (bold
and underlined), allotype G1m(f) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVDHHDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHSHYTQKSLSLSPGK
SEQ ID NO: 46: mature human Fc IgG1, Cys to Ser substitution (#), DHS triple mutation (bold
and underlined), allotype G1m(fa) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHSHYTQKSLSLSPGK
SEQ ID NO: 47: mature human Fc IgG1, Cys to Ser substitution (#), DHS triple mutation (bold
and underlined), allotype G1m(f) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVDHHDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHSHYTQKSLSLSPGK
SEQ ID NO: 48: mature human Fc IgG1, Cys to Ser substitution (#), DHS triple mutation (bold
and underlined), allotype G1m(fa) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVDHHDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHSHYTQKSLSLSPG
SEQ ID NO: 49: mature human Fc IgG1, Cys to Ser substitution (#), DHS triple mutation (bold
and underlined), allotype G1m(f) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYNSTYRVVSVLTVDHHDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHSHYTQKSLSLSPG
SEQ ID NO: 50: mature human Fc IgG1, Cys to Ser substitution (#), Asn to Ala substitution
(*), DHS triple mutation (bold and underlined), wherein X₇ is Asp or Glu and X₈ is Leu or Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIEKTI
SKAKGQPREPQVYTLPPSRX₇EX₈TKNQVSLTCLVKGFYPSDIAVEWESNGQPENNYKTTPPVLDSDGSF
FLYSKLTVDKSRWQQGNVFSCSVMHEALHSHYTQKSLSLSPGK
SEQ ID NO: 51: mature human Fc IgG1, Cys to Ser substitution (#), Asn to Ala substitution
(*), DHS triple mutation (bold and underlined), allotype G1m(fa) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHSHYTQKSLSLSPGK
SEQ ID NO: 52: mature human Fc IgG1, Cys to Ser substitution (#), Asn to Ala substitution
(*), DHS triple mutation (bold and underlined), allotype G1m(f) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIEKTI
SKAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHSHYTQKSLSLSPGK
SEQ ID NO: 53: mature human Fc IgG1, Cys to Ser substitution (#), Asn to Ala substitution
(*), DHS triple mutation (bold and underlined), allotype G1m(fa) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYA(*)STYRVVSVLTVDHHDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHSHYTQKSLSLSPGK
SEQ ID NO: 54: mature human Fc IgG1, Cys to Ser substitution (#), Asn to Ala substitution
(*), DHS triple mutation (bold and underlined), allotype G1m(f) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYA(*)STYRVVSVLTVDHHDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHSHYTQKSLSLSPGK
SEQ ID NO: 55: mature human Fc IgG1, Cys to Ser substitution (#), Asn to Ala substitution
(*), DHS triple mutation (bold and underlined), allotype G1m(fa) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHSHYTQKSLSLSPGK
SEQ ID NO: 56: mature human Fc IgG1, Cys to Ser substitution (#), Asn to Ala substitution
(*), DHS triple mutation (bold and underlined), allotype G1m(f) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYA(*)STYRVVSVLTVDHHDWLNGKEYKCKVSNKALPAPIEKTI
SKAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHSHYTQKSLSLSPGK
SEQ ID NO: 57: mature human Fc IgG1, Cys to Ser substitution (#), Asn to Ala substitution
(*), DHS triple mutation (bold and underlined), allotype G1m(fa) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYA(*)STYRVVSVLTVDHHDWLNGKEYKCKVSNKALPAPIEKTIS
KAKGQPREPQVYTLPPSRDELTKNQVSLTCLVKGFYPSDIAVEWESNGQPENNYKTTPPVLDSDGSFFL
YSKLTVDKSRWQQGNVFSCSVMHEALHSHYTQKSLSLSPG
SEQ ID NO: 58: mature human Fc IgG1, Cys to Ser substitution (#), Asn to Ala substitution
(*), DHS triple mutation (bold and underlined), allotype G1m(f) (bold italics)
VNHKPSNTKVDKKVEPKSS(#)DKTHTCPPCPAPELLGGPSVFLFPPKPKDTLMISRTPEVTCVVVDVSHE
DPEVKFNWYVDGVEVHNAKTKPREEQYA(*)STYRVVSVLTVDHHDWLNGKEYKCKVSNKALPAPIEKTIS
KAKGQPREPQVYTLPPSREEMTKNQVSLTCLVKGFYPSDIAVEWESNGQPENNYKTTPPVLDSDGSFFL
YSKLTVDKSRWQQGNVFSCSVMHEALHSHYTQKSLSLSPG
SEQ ID NO: 59: mature human Fc IgG1, J₁ is Asn or absent, J₂ is Lys or absent, and wherein
X₄ is Asn or Ala, X₅ is Leu or Asp, X₆ is Gln or His, X₇ is Asp or Glu, and X₈ is Leu or Met,
and X₁₀ is Asn or Ser
J₁VNHKPSNTKVDKKVEPKSCDKTHTCPPCPAPELLGGPSVFLFPPKPKDTLMISRTPEVTCVVVDVSHE
DPEVKFNWYVDGVEVHNAKTKPREEQYX₄STYRVVSVLTVX₅HX₆DWLNGKEYKCKVSNKALPAPIEKTIS
KAKGQPREPQVYTLPPSRX₇EX₈TKNQVSLTCLVKGFYPSDIAVEWESNGQPENNYKTTPPVLDSDGSFF
LYSKLTVDKSRWQQGNVFSCSVMHEALHX₁₀HYTQKSLSLSPGJ₂
SEQ ID NO: 60: mature human Fc IgG1, DHS triple mutation (bold and underlined), J₁ is Asn or
absent, J₂ is Lys or absent, and wherein X₄ is Asn or Ala, X₇ is Asp or Glu, and X₈ is Leu or
Met
J₁VNHKPSNTKVDKKVEPKSCDKTHTCPPCPAPELLGGPSVFLFPPKPKDTLMISRTPEVTCVVVDVSHE
DPEVKFNWYVDGVEVHNAKTKPREEQYX₄STYRVVSVLTVDHHDWLNGKEYKCKVSNKALPAPIEKTISK
AKGQPREPQVYTLPPSRX₇EX₈TKNQVSLTCLVKGFYPSDIAVEWESNGQPENNYKTTPPVLDSDGSFFL
YSKLTVDKSRWQQGNVFSCSVMHEALHSHYTQKSLSLSPGJ₂
SEQ ID NO: 61: mature human Fc IgG1, DHS triple mutation (bold and underlined), wherein X₄
is Asn or Ala, and X₇ is Asp or Glu, and X₈ is Leu or Met
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYX₄STYRVVSVLTVDHHDWLNGKEYKCKVSNKALPAPIEKTISKA
KGQPREPQVYTLPPSRX₇EX₈TKNQVSLTCLVKGFYPSDIAVEWESNGQPENNYKTTPPVLDSDGSFFLY
SKLTVDKSRWQQGNVFSCSVMHEALHSHYTQKSLSLSPGK
SEQ ID NO: 62: mature human Fc IgG1, DHS triple mutation (bold and underlined), wherein X₇
is Asp or Glu and X₈ is Leu or Met
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVDHHDWLNGKEYKCKVSNKALPAPIEKTISKA
KGQPREPQVYTLPPSRX₇EX₈TKNQVSLTCLVKGFYPSDIAVEWESNGQPENNYKTTPPVLDSDGSFFLY
SKLTVDKSRWQQGNVFSCSVMHEALHSHYTQKSLSLSPGK
SEQ ID NO: 63: mature human Fc IgG1, DHS triple mutation (bold and underlined), allotype
G1m(fa) (bold italics)
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVDHHDWLNGKEYKCKVSNKALPAPIEKTISKA
KGQPREPQVYTLPPSRDELTKNQVSLTCLVKGFYPSDIAVEWESNGQPENNYKTTPPVLDSDGSFFLYS
KLTVDKSRWQQGNVFSCSVMHEALHSHYTQKSLSLSPGK
SEQ ID NO: 64: mature human Fc IgG1, DHS triple mutation (bold and underlined), allotype
G1m(f) (bold italics)
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVDHHDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHSHYTQKSLSLSPGK
SEQ ID NO: 65: mature human Fc IgG1, DHS triple mutation (bold and underlined), allotype
G1m(fa) (bold italics)
VNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVDHHDWLNGKEYKCKVSNKALPAPIEKTISKAK
GQPREPQVYTLPPSRDELTKNQVSLTCLVKGFYPSDIAVEWESNGQPENNYKTTPPVLDSDGSFFLYSK
LTVDKSRWQQGNVFSCSVMHEALHSHYTQKSLSLSPGK
SEQ ID NO: 66: mature human Fc IgG1, DHS triple mutation (bold and underlined), allotype
G1m(f) (bold italics)
VNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVDHHDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSK
LTVDKSRWQQGNVFSCSVMHEALHSHYTQKSLSLSPGK
SEQ ID NO: 67: mature human Fc IgG1, DHS triple mutation (bold and underlined), allotype
G1m(fa) (bold italics)
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVDHHDWLNGKEYKCKVSNKALPAPIEKTISKA
KGQPREPQVYTLPPSRDELTKNQVSLTCLVKGFYPSDIAVEWESNGQPENNYKTTPPVLDSDGSFFLYS
KLTVDKSRWQQGNVFSCSVMHEALHSHYTQKSLSLSPGK
SEQ ID NO: 68: mature human Fc IgG1, DHS triple mutation (bold and underlined), allotype
G1m(f) (bold italics)
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVDHHDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHSHYTQKSLSLSPGK
SEQ ID NO: 69: mature human Fc IgG1, DHS triple mutation (bold and underlined), allotype
G1m(fa) (bold italics)
VNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVDHHDWLNGKEYKCKVSNKALPAPIEKTISKAK
GQPREPQVYTLPPSRDELTKNQVSLTCLVKGFYPSDIAVEWESNGQPENNYKTTPPVLDSDGSFFLYSK
LTVDKSRWQQGNVFSCSVMHEALHSHYTQKSLSLSPG
SEQ ID NO: 70: mature human Fc IgG1, DHS triple mutation (bold and underlined), allotype
G1m(f) (bold italics)
VNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVDHHDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSK
LTVDKSRWQQGNVFSCSVMHEALHSHYTQKSLSLSPG
SEQ ID NO: 71: mature human Fc IgG1, Asn to Ala substitution (*), DHS triple mutation (bold
and underlined), wherein X₇ is Asp or Glu and X₈ is Leu or Met
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYA(*)STYRVVSVLTVDHHDWLNGKEYKCKVSNKALPAPIEKTISK
AKGQPREPQVYTLPPSRX₇EX₈TKNQVSLTCLVKGFYPSDIAVEWESNGQPENNYKTTPPVLDSDGSFFL
YSKLTVDKSRWQQGNVFSCSVMHEALHSHYTQKSLSLSPGK
SEQ ID NO: 72: mature human Fc IgG1, Asn to Ala substitution (*), DHS triple mutation (bold
and underlined), allotype G1m(fa) (bold italics)
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYA(*)STYRVVSVLTVDHHDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHSHYTQKSLSLSPGK
SEQ ID NO: 73: mature human Fc IgG1, Asn to Ala substitution (*), DHS triple mutation (bold
and underlined), allotype G1m(f) (bold italics)
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYA(*)STYRVVSVLTVDHHDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHSHYTQKSLSLSPGK
SEQ ID NO: 74: mature human Fc IgG1, Asn to Ala substitution (*), DHS triple mutation (bold
and underlined), allotype G1m(fa) (bold italics)
VNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYA(*)STYRVVSVLTVDHHDWLNGKEYKCKVSNKALPAPIEKTISKA
KGQPREPQVYTLPPSRDELTKNQVSLTCLVKGFYPSDIAVEWESNGQPENNYKTTPPVLDSDGSFFLYS
KLTVDKSRWQQGNVFSCSVMHEALHSHYTQKSLSLSPGK
SEQ ID NO: 75: mature human Fc IgG1, Asn to Ala substitution (*), DHS triple mutation (bold
and underlined), allotype G1m(f) (bold italics)
VNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYA(*)STYRVVSVLTVDHHDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHSHYTQKSLSLSPGK
SEQ ID NO: 76: mature human Fc IgG1, Asn to Ala substitution (*), DHS triple mutation (bold
and underlined), allotype G1m(fa) (bold italics)
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYA(*)STYRVVSVLTVDHHDWLNGKEYKCKVSNKALPAPIEKTISK
AKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHSHYTQKSLSLSPGK
SEQ ID NO: 77: mature human Fc IgG1, Asn to Ala substitution (*), DHS triple mutation (bold
and underlined), allotype G1m(f) (bold italics)
NVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYA(*)STYRVVSVLTVDHHDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHSHYTQKSLSLSPGK
SEQ ID NO: 78: mature human Fc IgG1, Asn to Ala substitution (*), DHS triple mutation (bold
and underlined), allotype G1m(fa) (bold italics)
VNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYA(*)STYRVVSVLTVDHHDWLNGKEYKCKVSNKALPAPIEKTISKA
KGQPREPQVYTLPPSRDELTKNQVSLTCLVKGFYPSDIAVEWESNGQPENNYKTTPPVLDSDGSFFLYS
KLTVDKSRWQQGNVFSCSVMHEALHSHYTQKSLSLSPG
SEQ ID NO: 79: mature human Fc IgG1, Asn to Ala substitution (*), DHS triple mutation (bold
and underlined), allotype G1m(f) (bold italics)
VNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYA(*)STYRVVSVLTVDHHDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLYS
KLTVDKSRWQQGNVFSCSVMHEALHSHYTQKSLSLSPG
SEQ ID NO: 80: mature human IgG1 Fc, Cys to Ser substitution (#), allotype G1m(f) (bold
italics), Asn to Ala substitution (*)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYA(*)STYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 81: mature human IgG1 Fc, Cys to Ser substitution (#), allotype G1m(fa) (bold
italics), Asn to Ala substitution (*)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYA(*)STYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 82: mature human IgG1 Fc, Cys to Ser substitution (#), allotype G1m(f) (bold
italics), YTE triple mutation (bold and underlined), Asn to Ala substitution (*)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYA(*)STYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 83: mature human IgG1 Fc, Cys to Ser substitution (#), allotype G1m(fa) (bold
italics), YTE triple mutation (bold and underlined), Asn to Ala substitution (*)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYA(*)STYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPG

In some embodiments, the variant Fc domain includes an amino acid substitution at position 246 (e.g., K246X where X is any amino acid that is not Lys, such as K246S, K246G, K246A, K246T, K246N, K246Q, K246R, K246H, K246E, or K246DC220S). In some embodiments, the variant Fc domain monomer includes at least the following mutations K246X, M252Y, S254T, and T256E, where X is not Lys. In some embodiments, the variant Fc domain monomer includes at least the following mutations K246X, V309D, Q311H, and N434S, where X is not Lys. In some embodiments, the variant Fc domain monomer includes at least the following mutations K246X, M428L, and N434S, where X is not Lys. In some embodiments, the variant Fc domain further includes a mutation of position 220, e.g., a C220S mutation. Amino acid substitutions are relative to a wild-type Fc monomer amino acid sequence, e.g., wild-type human IgG1 or IgG2.

In some embodiments, a variant Fc domain monomer includes a sequence that is at least 95% identical (e.g., 97%, 99%, or 99.5% identical) to the sequence of any one of SEQ ID Nos: 84-112 and 115-120 shown below. In some embodiments, a variant Fc domain monomer includes the sequence of any one of SEQ ID Nos: 84-112 and 115-120 shown below.

In some embodiments, a variant Fc domain monomer includes at least the following mutations K246X, M252Y, S254T, and T256E, where X is not Lys. In some embodiments, a variant Fc domain monomer includes at least the following mutations K246X, V309D, Q311H, and N434S, where X is not Lys. In some embodiments, a variant Fc domain monomer includes at least the following mutations K246X, M428L, and N434S, where X is not Lys. In some embodiments, the substitution at K246X is selected from Ser, Gly, Ala, Thr, Asn, Gln, Arg, His, Glu, or Asp. In some embodiments, the substitution at K246X is Ser.

SEQ ID NO: 84: mature human IgG1 Fc; X₁ (position 201) is Asn or absent; X₂ (position 220)
is Cys or Ser; X₃ (position 246) is Ser, Gly, Ala, Thr, Asn, Gln, Arg, His, Glu, or Asp; X₄
(position 252) is Met or Tyr; X₅ (position 254) is Ser or Thr; X₆ (position 256) is Thr or
Glu; X₇ (position 297) is Asn or Ala; X₈ (position 309) is Leu or Asp; X₉ (position 311) is
Gln or His; X₁₀ (position 356) is Asp or Glu; and X₁₁ (position 358) is Leu or Met; X₁₂
(position 428) is Met or Leu; X₁₃ (position 434) is Asn or Ser; X₁₄ (position 447) is Lys or
absent; N-terminal Fab residues are underlined; hinge residues are italicized
X₁VNHKPSNTKVDKKVEPKSX₂DKTHTCPPCPAPELLGGPSVFLFPPX₃PKDTLX₄IX₅RX₆PEVTCVVVDVS
HEDPEVKFNWYVDGVEVHNAKTKPREEQYX₇STYRVVSVLTVX₈HX₉DWLNGKEYKCKVSNKALPAPIEK
TISKAKGQPREPQVYTLPPSRX₁₀EX₁₁TKNQVSLTCLVKGFYPSDIAVEWESNGQPENNYKTTPPVLDSD
GSFFLYSKLTVDKSRWQQGNVFSCSVX₁₂HEALHX₁₃HYTQKSLSLSPGX₁₄
SEQ ID NO: 85: mature human IgG1 Fc; Cys to Ser substitution (#); X₁ is Asn or absent; X₂ is
Ser, Gly, Ala, Thr, Asn, Gln, Arg, His, Glu, or Asp; X₃ is Asn or Ala; X₄ is Asp or Glu; and
X₅ is Leu or Met; X₆ is Lys or absent; N-terminal Fab residues are underlined; hinge residues
are italicized
X₁VNHKPSNTKVDKKVEPKSS(#)DKTHTCPPCPAPELLGGPSVFLFPPX₂PKDTLMISRTPEVTCVVVDVS
HEDPEVKFNWYVDGVEVHNAKTKPREEQYX₃STYRVVSVLTVLHQDWLNGKEYKCKVSNKALPAPIEKTI
SKAKGQPREPQVYTLPPSRX₄EX₅TKNQVSLTCLVKGFYPSDIAVEWESNGQPENNYKTTPPVLDSDGSF
FLYSKLTVDKSRWQQGNVFSCSVMHEALHNHYTQKSLSLSPGX₆
SEQ ID NO: 86: mature human IgG1 Fc; Cys to Ser substitution (#); X₁ is Ser, Gly, Ala, Thr,
Asn, Gln, Arg, His, Glu, or Asp; X₂ is Asn or Ala; X₃ is Asp or Glu; and X₄ is Leu or Met; N-
terminal Fab residues are underlined; hinge residues are italicized
NVNHKPSNTKVDKKVEPKSS(#)DKTHTCPPCPAPELLGGPSVFLFPPX₁PKDTLMISRTPEVTCVVVDVS
HEDPEVKFNWYVDGVEVHNAKTKPREEQYX₂STYRVVSVLTVLHQDWLNGKEYKCKVSNKALPAPIEKTI
SKAKGQPREPQVYTLPPSRX₃EX₄TKNQVSLTCLVKGFYPSDIAVEWESNGQPENNYKTTPPVLDSDGSF
FLYSKLTVDKSRWQQGNVFSCSVMHEALHNHYTQKSLSLSPG
SEQ ID NO: 87: mature human IgG1 Fc; Cys to Ser substitution (#); Lys to Ser substitution
(*); X₁ is Asp or Glu; and X₂ is Leu or Met; N-terminal Fab residues are underlined; hinge
residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSRX₁EX₂TKNQVSLTCLVKGFYPSDIAVEWESNGQPENNYKTTPPVLDSDGS
FFLYSKLTVDKSRWQQGNVFSCSVMHEALHNHYTQKSLSLSPG
SEQ ID NO: 88: mature human IgG1 Fc; Cys to Ser substitution (#); Lys to Ser substitution
(*); allotype G1m(fa) (bold italics); N-terminal Fab residues are underlined; hinge residues
are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 89: mature human IgG1 Fc; Cys to Ser substitution (#); Lys to Ser substitution
(*); allotype G1m(f) (bold italics); N-terminal Fab residues are underlined; hinge residues
are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPG
SEQ ID NO: 90: mature human IgG1 Fc; Cys to Ser substitution (#); Asn to Ala substitution
(^{∧}); X₁ is Ser, Gly, Ala, Thr, Asn, Gln, Arg, His, Glu, or Asp; X₂ is Asp or Glu; and X₃ is
Leu or Met; N-terminal Fab residues are underlined; hinge residues are italicized
NVNHKPSNTKVDKKVEPKSS(#)DKTHTCPPCPAPELLGGPSVFLFPPX₁PKDTLMISRTPEVTCVVVDVS
HEDPEVKFNWYVDGVEVHNAKTKPREEQYA(^{∧})STYRVVSVLTVLHQDWLNGKEYKCKVSNKALPAPIEK
TISKAKGQPREPQVYTLPPSRX₂EX₃TKNQVSLTCLVKGFYPSDIAVEWESNGQPENNYKTTPPVLDSDG
SFFLYSKLTVDKSRWQQGNVFSCSVMHEALHNHYTQKSLSLSPG
SEQ ID NO: 91: mature human IgG1 Fc; Cys to Ser substitution (#); Lys to Ser substitution
(*); Asn to Ala substitution (^{∧}); X₁ is Asp or Glu; and X₂ is Leu or Met; N-terminal Fab
residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYA(^{∧})STYRVVSVLTVLHQDWLNGKEYKCKVSNKALPAPIE
KTISKAKGQPREPQVYTLPPSRX₁EX₂TKNQVSLTCLVKGFYPSDIAVEWESNGQPENNYKTTPPVLDSD
GSFFLYSKLTVDKSRWQQGNVFSCSVMHEALHNHYTQKSLSLSPG
SEQ ID NO: 92: mature human IgG1 Fc; Cys to Ser substitution (#); YTE triple mutation (bold
and underlined); X₁ is Asn or absent; X₂ is Ser, Gly, Ala, Thr, Asn, Gln, Arg, His, Glu, or
Asp; X₃ is Asn or Ala; X₄ is Asp or Glu; and X₅ is Leu or Met; X₆ is Lys or absent; N-terminal
Fab residues are underlined; hinge residues are italicized
X₁VNHKPSNTKVDKKVEPKSS(#)DKTHTCPPCPAPELLGGPSVFLFPPX₂PKDTLYITREPEVTCVVVDVS
HEDPEVKFNWYVDGVEVHNAKTKPREEQYX₃STYRVVSVLTVLHQDWLNGKEYKCKVSNKALPAPIEKTI
SKAKGQPREPQVYTLPPSRX₄EX₅TKNQVSLTCLVKGFYPSDIAVEWESNGQPENNYKTTPPVLDSDGSF
FLYSKLTVDKSRWQQGNVFSCSVMHEALHNHYTQKSLSLSPGX₆
SEQ ID NO: 93: mature human IgG1 Fc; Cys to Ser substitution (#); YTE triple mutation (bold
and underlined); X₁ is Ser, Gly, Ala, Thr, Asn, Gln, Arg, His, Glu, or Asp; X₂ is Asn or Ala;
X₃ is Asp or Glu; and X₄ is Leu or Met; N-terminal Fab residues are underlined; hinge residues
are italicized
NVNHKPSNTKVDKKVEPKSS(#)DKTHTCPPCPAPELLGGPSVFLFPPX₁PKDTLYITREPEVTCVVVDVSH
EDPEVKFNWYVDGVEVHNAKTKPREEQYX₂STYRVVSVLTVLHQDWLNGKEYKCKVSNKALPAPIEKTIS
KAKGQPREPQVYTLPPSRX₃EX₄TKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 94: mature human IgG1 Fc; Cys to Ser substitution (#); Lys to Ser substitution
(*); YTE triple mutation (bold and underlined); X₁ is Asp or Glu; and X₂ is Leu or Met; N-
terminal Fab residues are underlined; hinge residues are italicized
NVNHKPSNTKVDKKVEPKSS(#)DKTHTCPPCPAPELLGGPSVFLFPPS(*)PKDTLYITREPEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSRX₁EX₂TKNQVSLTCLVKGFYPSDIAVEWESNGQPENNYKTTPPVLDSDGSF
FLYSKLTVDKSRWQQGNVFSCSVMHEALHNHYTQKSLSLSPG
SEQ ID NO: 95: mature human IgG1 Fc; Cys to Ser substitution (#); Lys to Ser substitution
(*); YTE triple mutation (bold and underlined); allotype G1m(fa) (bold italics); N-terminal
Fab residues are underlined; hinge residues are italicized
NVNHKPSNTKVDKKVEPKSS(#)DKTHTCPPCPAPELLGGPSVFLFPPS(*)PKDTLYITREPEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 96: mature human IgG1 Fc; Cys to Ser substitution (#); Lys to Ser substitution
(*); YTE triple mutation (bold and underlined); allotype G1m(f) (bold italics); N-terminal
Fab residues are underlined; hinge residues are italicized
NVNHKPSNTKVDKKVEPKSS(#)DKTHTCPPCPAPELLGGPSVFLFPPS(*)PKDTLYITREPEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 97: mature human IgG1 Fc; Cys to Ser substitution (#); Asn to Ala substitution
(^{∧}); YTE triple mutation (bold and underlined); X₁ is Ser, Gly, Ala, Thr, Asn, Gln, Arg, His,
Glu, or Asp; X₂ is Asp or Glu; and X₃ is Leu or Met; N-terminal Fab residues are underlined;
hinge residues are italicized
NVNHKPSNTKVDKKVEPKSS(#)DKTHTCPPCPAPELLGGPSVFLFPPX₁PKDTLYITREPEVTCVVVDVSH
EDPEVKFNWYVDGVEVHNAKTKPREEQYA(^{∧})STYRVVSVLTVLHQDWLNGKEYKCKVSNKALPAPIEKTI
SKAKGQPREPQVYTLPPSRX₂EX₃TKNQVSLTCLVKGFYPSDIAVEWESNGQPENNYKTTPPVLDSDGSF
FLYSKLTVDKSRWQQGNVFSCSVMHEALHNHYTQKSLSLSPG
SEQ ID NO: 98: mature human IgG1 Fc; Cys to Ser substitution (#); Lys to Ser substitution
(*); Asn to Ala substitution (^{∧}); YTE triple mutation (bold and underlined); X₁ is Asp or Glu;
and X₂ is Leu or Met; N-terminal Fab residues are underlined; hinge residues are italicized
NVNHKPSNTKVDKKVEPKSS(#)DKTHTCPPCPAPELLGGPSVFLFPPS(*)PKDTLYITREPEVTCVVVDVS
HEDPEVKFNWYVDGVEVHNAKTKPREEQYA(^{∧})STYRVVSVLTVLHQDWLNGKEYKCKVSNKALPAPIEK
TISKAKGQPREPQVYTLPPSRX₁EX₂TKNQVSLTCLVKGFYPSDIAVEWESNGQPENNYKTTPPVLDSDG
SFFLYSKLTVDKSRWQQGNVFSCSVMHEALHNHYTQKSLSLSPG
SEQ ID NO: 99: mature human IgG1 Fc; Cys to Ser substitution (#); DHS triple mutation (bold
and underlined); X₁ is Asn or absent; X₂ is Ser, Gly, Ala, Thr, Asn, Gln, Arg, His, Glu, or
Asp; X₃ is Asn or Ala; X₄ is Asp or Glu; and X₅ is Leu or Met; X₆ is Lys or absent; N-terminal
Fab residues are underlined; hinge residues are italicized
X₁VNHKPSNTKVDKKVEPKSS(#)DKTHTCPPCPAPELLGGPSVFLFPPX₂PKDTLMISRTPEVTCVVVDVS
HEDPEVKFNWYVDGVEVHNAKTKPREEQYX₃STYRVVSVLTVDHHDWLNGKEYKCKVSNKALPAPIEKTI
SKAKGQPREPQVYTLPPSRX₄EX₅TKNQVSLTCLVKGFYPSDIAVEWESNGQPENNYKTTPPVLDSDGSF
FLYSKLTVDKSRWQQGNVFSCSVMHEALHSHYTQKSLSLSPGX₆
SEQ ID NO: 100: mature human IgG1 Fc; Cys to Ser substitution (#); DHS triple mutation (bold
and underlined); X₁ is Ser, Gly, Ala, Thr, Asn, Gln, Arg, His, Glu, or Asp; X₂ is Asn or Ala;
X₃ is Asp or Glu; and X₄ is Leu or Met; N-terminal Fab residues are underlined; hinge
residues are italicized
NVNHKPSNTKVDKKVEPKSS(#)DKTHTCPPCPAPELLGGPSVFLFPPX₁PKDTLMISRTPEVTCVVVDVS
HEDPEVKFNWYVDGVEVHNAKTKPREEQYX₂STYRVVSVLTVDHHDWLNGKEYKCKVSNKALPAPIEKTI
SKAKGQPREPQVYTLPPSRX₃EX₄TKNQVSLTCLVKGFYPSDIAVEWESNGQPENNYKTTPPVLDSDGSF
FLYSKLTVDKSRWQQGNVFSCSVMHEALHSHYTQKSLSLSPG
SEQ ID NO: 101: mature human IgG1 Fc; Cys to Ser substitution (#); Lys to Ser substitution
(*); DHS triple mutation (bold and underlined); X₁ is Asp or Glu; and X₂ is Leu or Met; N-
terminal Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVDHHDWLNGKEYKCKVSNKALPAPIEK
TISKAKGQPREPQVYTLPPSRX₁EX₂TKNQVSLTCLVKGFYPSDIAVEWESNGQPENNYKTTPPVLDSDG
SFFLYSKLTVDKSRWQQGNVFSCSVMHEALHSHYTQKSLSLSPG
SEQ ID NO: 102: mature human IgG1 Fc; Cys to Ser substitution (#); Lys to Ser substitution
(*); DHS triple mutation (bold and underlined); allotype G1m(fa) (bold italics); N-terminal
Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVDHHDWLNGKEYKCKVSNKALPAPIEK
TISKAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHSHYTQKSLSLSPG
SEQ ID NO: 103: mature human IgG1 Fc; Cys to Ser substitution (#); Lys to Ser substitution
(*); DHS triple mutation (bold and underlined); allotype G1m(f) (bold italics); N-terminal
Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVDHHDWLNGKEYKCKVSNKALPAPIEK
TISKAKGQPREPQVYTLPPSREEMTKNQVSLTCLVKGFYPSDIAVEWESNGQPENNYKTTPPVLDSDGS
FFLYSKLTVDKSRWQQGNVFSCSVMHEALHSHYTQKSLSLSPG
SEQ ID NO: 104: mature human IgG1 Fc; Cys to Ser substitution (#); Asn to Ala substitution
(^{∧}); DHS triple mutation (bold and underlined); X₁ is Ser, Gly, Ala, Thr, Asn, Gln, Arg, His,
Glu, or Asp; X₂ is Asp or Glu; and X₃ is Leu or Met; N-terminal Fab residues are underlined;
hinge residues are italicized
NVNHKPSNTKVDKKVEPKSS(#)DKTHTCPPCPAPELLGGPSVFLFPPX₁PKDTLMISRTPEVTCVVVDVS
HEDPEVKFNWYVDGVEVHNAKTKPREEQYA(^{∧})STYRVVSVLTVDHHDWLNGKEYKCKVSNKALPAPIEK
TISKAKGQPREPQVYTLPPSRX₂EX₃TKNQVSLTCLVKGFYPSDIAVEWESNGQPENNYKTTPPVLDSDG
SFFLYSKLTVDKSRWQQGNVFSCSVMHEALHSHYTQKSLSLSPG
SEQ ID NO: 105: mature human IgG1 Fc; Cys to Ser substitution (#); Lys to Ser substitution
(*); Asn to Ala substitution (^{∧}); DHS triple mutation (bold and underlined); X₁ is Asp or
Glu; and X₂ is Leu or Met; N-terminal Fab residues are underlined; hinge residues are
italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYA(^{∧})STYRVVSVLTVDHHDWLNGKEYKCKVSNKALPAPIE
KTISKAKGQPREPQVYTLPPSRX₁EX₂TKNQVSLTCLVKGFYPSDIAVEWESNGQPENNYKTTPPVLDSD
GSFFLYSKLTVDKSRWQQGNVFSCSVMHEALHSHYTQKSLSLSPG
SEQ ID NO: 106: mature human IgG1 Fc; Cys to Ser substitution (#); LS double mutation (bold
and underlined); X₁ is Asn or absent; X₂ is Ser, Gly, Ala, Thr, Asn, Gln, Arg, His, Glu, or
Asp; X₃ is Asn or Ala; X₄ is Asp or Glu; and X₅ is Leu or Met; X₆ is Lys or absent; N-terminal
Fab residues are underlined; hinge residues are italicized
X₁VNHKPSNTKVDKKVEPKSS(#)DKTHTCPPCPAPELLGGPSVFLFPPX₂PKDTLMISRTPEVTCVVVDVS
HEDPEVKFNWYVDGVEVHNAKTKPREEQYX₃STYRVVSVLTVLHQDWLNGKEYKCKVSNKALPAPIEKTI
SKAKGQPREPQVYTLPPSRX₄EX₅TKNQVSLTCLVKGFYPSDIAVEWESNGQPENNYKTTPPVLDSDGSF
FLYSKLTVDKSRWQQGNVFSCSVLHEALHSHYTQKSLSLSPGX₆
SEQ ID NO: 107: mature human IgG1 Fc; Cys to Ser substitution (#); LS double mutation (bold
and underlined); X₁ is Ser, Gly, Ala, Thr, Asn, Gln, Arg, His, Glu, or Asp; X₂ is Asn or Ala;
X₃ is Asp or Glu; and X₄ is Leu or Met; N-terminal Fab residues are underlined; hinge
residues are italicized
NVNHKPSNTKVDKKVEPKSS(#)DKTHTCPPCPAPELLGGPSVFLFPPX₁PKDTLMISRTPEVTCVVVDVS
HEDPEVKFNWYVDGVEVHNAKTKPREEQYX₂STYRVVSVLTVLHQDWLNGKEYKCKVSNKALPAPIEKTI
SKAKGQPREPQVYTLPPSRX₃EX₄TKNQVSLTCLVKGFYPSDIAVEWESNGQPENNYKTTPPVLDSDGSF
FLYSKLTVDKSRWQQGNVFSCSVLHEALHSHYTQKSLSLSPG
SEQ ID NO: 108: mature human IgG1 Fc; Cys to Ser substitution (#); Lys to Ser substitution
(*); LS double mutation (bold and underlined); X₁ is Asp or Glu; and X₂ is Leu or Met; N-
terminal Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSRX₁EX₂TKNQVSLTCLVKGFYPSDIAVEWESNGQPENNYKTTPPVLDSDGS
FFLYSKLTVDKSRWQQGNVFSCSVLHEALHSHYTQKSLSLSPG
SEQ ID NO: 109: mature human IgG1 Fc; Cys to Ser substitution (#); Lys to Ser substitution
(*); LS double mutation (bold and underlined); allotype G1m(fa) (bold italics); N-terminal
Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTPPVLDSDGSFF
LYSKLTVDKSRWQQGNVFSCSVLHEALHSHYTQKSLSLSPG
SEQ ID NO: 110: mature human IgG1 Fc; Cys to Ser substitution (#); Lys to Ser substitution
(*); LS double mutation (bold and underlined); allotype G1m(f) (bold italics); N-terminal
Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNGQPENNYKTTPPVLDSDGSF
FLYSKLTVDKSRWQQGNVFSCSVLHEALHSHYTQKSLSLSPG
SEQ ID NO: 111: mature human IgG1 Fc; Cys to Ser substitution (#); Asn to Ala substitution
(^{∧}); LS double mutation (bold and underlined); X₁ is Ser, Gly, Ala, Thr, Asn, Gln, Arg, His,
Glu, or Asp; X₂ is Asp or Glu; and X₃ is Leu or Met; N-terminal Fab residues are underlined;
hinge residues are italicized
NVNHKPSNTKVDKKVEPKSS(#)DKTHTCPPCPAPELLGGPSVFLFPPX₁PKDTLMISRTPEVTCVVVDVS
HEDPEVKFNWYVDGVEVHNAKTKPREEQYA(^{∧})STYRVVSVLTVLHQDWLNGKEYKCKVSNKALPAPIEK
TISKAKGQPREPQVYTLPPSRX₂EX₃TKNQVSLTCLVKGFYPSDIAVEWESNGQPENNYKTTPPVLDSDG
SFFLYSKLTVDKSRWQQGNVFSCSVLHEALHSHYTQKSLSLSPG
SEQ ID NO: 112: mature human IgG1 Fc; Cys to Ser substitution (#); Lys to Ser substitution
(*); Asn to Ala substitution (^{∧}); LS double mutation (bold and underlined); X₁ is Asp or Glu;
and X₂ is Leu or Met; N-terminal Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYA(^{∧})STYRVVSVLTVLHQDWLNGKEYKCKVSNKALPAPIE
KTISKAKGQPREPQVYTLPPSRX₁EX₂TKNQVSLTCLVKGFYPSDIAVEWESNGQPENNYKTTPPVLDSD
GSFFLYSKLTVDKSRWQQGNVFSCSVLHEALHSHYTQKSLSLSPG
SEQ ID NO: 113: Palivizumab full length antibody; anti-RSV IgG; leader sequence underlined
Heavy chain:
MGWSCIILFLVATATGVHSQVTLRESGPALVKPTQTLTLTCTFSGFSLSTSGMSVGWIRQPPGKALEWLA
DIWWDDKKDYNPSLKSRLTISKDTSKNQVVLKVTNMDPADTATYYCARSMITNWYFDVWGAGTTVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKRVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPG
SEQ ID NO: 114: Palivizumab full length antibody; anti-RSV IgG; leader sequence underlined
Light chain:
MGWSCIILFLVATATGVHSDIQMTQSPSTLSASVGDRVTITCKCQLSVGYMHWYQQKPGKAPKLLIYDTS
KLASGVPSRFSGSGSGTEFTLTISSLQPDDFATYYCFQGSGYPFTFGGGTKLEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
SEQ ID NO: 115: mature human IgG1 Fc; Cys to Ser substitution (#); Asn to Gln substitution
(^{∧}); allotype G1m(fa) (bold italics); N-terminal Fab residues are underlined; hinge residues
are italicized
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYQ(^{∧})STYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 116: mature human IgG1 Fc; Cys to Ser substitution (#); Asn to Gln substitution
(^{∧}); allotype G1m(f) (bold italics); N-terminal Fab residues are underlined; hinge residues
are italicized
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYQ(^{∧})STYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 117: mature human IgG1 Fc; Cys to Ser substitution (#); Lys to Ser substitution
(*); LS double mutation (bold and underlined); Asn to Gln substitution (^{∧}); allotype G1m(f)
(bold italics); N-terminal Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYQ(^{∧})STYRVVSVLTVLHQDWLNGKEYKCKVSNKALPAPIE
KTISKAKGQPREPQVYTLPPSREEMTKNQVSLTCLVKGFYPSDIAVEWESNGQPENNYKTTPPVLDSDG
SFFLYSKLTVDKSRWQQGNVFSCSVLHEALHSHYTQKSLSLSPG
SEQ ID NO: 118: mature human IgG1 Fc; Cys to Ser substitution (#); LS double mutation (bold
and underlined); Asn to Gln substitution (^{∧}); X₁ is Ser, Gly, Ala, Thr, Asn, Gln, Arg, His,
Glu, or Asp; X₂ is Asp or Glu; and X₃ is Leu or Met; N-terminal Fab residues are underlined;
hinge residues are italicized
NVNHKPSNTKVDKKVEPKSS(#)DKTHTCPPCPAPELLGGPSVFLFPPX₁PKDTLMISRTPEVTCVVVDVS
HEDPEVKFNWYVDGVEVHNAKTKPREEQYQ(^{∧})STYRVVSVLTVLHQDWLNGKEYKCKVSNKALPAPIEK
TISKAKGQPREPQVYTLPPSRX₂EX₃TKNQVSLTCLVKGFYPSDIAVEWESNGQPENNYKTTPPVLDSDG
SFFLYSKLTVDKSRWQQGNVFSCSVLHEALHSHYTQKSLSLSPG
SEQ ID NO: 119: mature human IgG1 Fc; Cys to Ser substitution (#); Lys to Ser substitution
(*); Asn to Gln substitution (^{∧}); LS double mutation (bold and underlined); X₁ is Asp or Glu;
and X₂ is Leu or Met; N-terminal Fab residues are underlined; hinge residues are italicized
NVNHKPSNTKVDKKVEPKSS(#)DKTHTCPPCPAPELLGGPSVFLFPPS(*)PKDTLMISRTPEVTCVVVDV
SHEDPEVKFNWYVDGVEVHNAKTKPREEQYQ(^{∧})STYRVVSVLTVLHQDWLNGKEYKCKVSNKALPAPIE
KTISKAKGQPREPQVYTLPPSRX₁EX₂TKNQVSLTCLVKGFYPSDIAVEWESNGQPENNYKTTPPVLDSD
GSFFLYSKLTVDKSRWQQGNVFSCSVLHEALHSHYTQKSLSLSPG
SEQ ID NO: 120: mature human IgG1 Fc; Cys to Ser substitution (#); Asn to Gln substitution
(^{∧}); allotype G1m(f) (bold italics); N-terminal Fab residues are underlined; hinge residues
are italicized
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYQ(^{∧})STYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPGK

As defined herein, an Fc domain includes two Fc domain monomers that are dimerized by the interaction between the C_{H}3 antibody constant domains, as well as one or more disulfide bonds that form between the hinge domains of the two dimerizing Fc domain monomers. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present disclosure binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

In some embodiments, the Fc domain monomer or Fc domain of the disclosure is an aglycosylated Fc domain monomer or Fc domain (e.g., an Fc domain monomer or an Fc domain that maintains engagement to an Fc receptor (e.g., FcRn). For example, the Fc domain is an aglycosylated IgG1 variants that maintains engagement to an Fc receptor (e.g., an IgG1 having an amino acid substitution at N297 and/or T299 of the glycosylation motif). Exemplary aglycosylated Fc domains and methods for making aglycosylated Fc domains are known in the art, for example, as described in Sazinsky S. L. et al., Aglycosylated immunoglobulin G1 variants productively engage activating Fc receptors, PNAS, 2008, 105(51):20167-20172, which is incorporated herein in its entirety.

In some embodiments, the Fc domain or Fc domain monomer of the disclosure is engineered to enhance binding to the neonatal Fc receptor (FcRn). For example, the Fc domain may include the triple mutation corresponding to M252Y/S254T/T256E (YTE) (e.g., an IgG1, such as a human or humanized IgG1 having a YTE mutation). The Fc domain may include the single mutant corresponding to N434H (e.g., an IgG1, such as a human or humanized IgG1 having an N434H mutation). The Fc domain may include the single mutant corresponding to C220S (e.g., and IgG1, such as a human or humanized IgG1 having a C220S mutation). The Fc domain may include a quadruple mutant corresponding to C220S/L309D/Q311H/N434S (CDHS) (e.g., an IgG1, such as a human or humanized IgG1 having a DHS mutation). The Fc domain may include a triple mutant corresponding to L309D/Q311H/N434S (DHS) (e.g., an IgG1, such as a human or humanized IgG1 having a DHS mutation). The Fc domain may include a combination of one or more of the above-described mutations that enhance binding to the FcRn. Enhanced binding to the FcRn may increase the half-life Fc domain-containing conjugate. For example, incorporation of one or more amino acid mutations that increase binding to the FcRn (e.g., a YTE mutation, an LS mutation, or an N434H mutation) may increase the half-life of the conjugate by 5%, 10%, 15%, 20%, 30%, 40%, 50%, 60%, 70%, 80%, 90%, 100%, 200%, 300%, 400%, 500% or more relative to a conjugate having the corresponding Fc domain without the mutation that enhances FcRn binding. Exemplary Fc domains with enhanced binding to the FcRN and methods for making Fc domains having enhanced binding to the FcRN are known in the art, for example, as described in Maeda, A. et al., Identification of human IgG1 variant with enhanced FcRn binding and without increased binding to rheumatoid factor autoantibody, MABS, 2017, 9(5):844-853, which is incorporated herein in its entirety. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID Nos: 1-112 and 115-120 may be mutated to include a YTE mutation, an LS mutation, and/or an N434H mutation by mutating the “corresponding residues” of the amino acid sequence.

In some embodiments, the Fc domain or Fc domain monomer of the disclosure has the sequence of any one of SEQ ID NOs: 1-112 and 115-120 may further include additional amino acids at the N-terminus (Xaa)x and/or additional amino acids at the C-terminus (Xaa)z, wherein Xaa is any amino acid and x and z are a whole number greater than or equal to zero, generally less than 100, preferably less than 10 and more preferably 0, 1, 2, 3, 4, or 5. For example, the additional amino acids may be a single amino acid on the C-terminus corresponding to Lys330 of IgG1.

In some embodiments, the Fc domain monomer includes less than about 300 amino acid residues (e.g., less than about 300, less than about 295, less than about 290, less than about 285, less than about 280, less than about 275, less than about 270, less than about 265, less than about 260, less than about 255, less than about 250, less than about 245, less than about 240, less than about 235, less than about 230, less than about 225, or less than about 220 amino acid residues). In some embodiments, the Fc domain monomer is less than about 40 kDa (e.g., less than about 35 kDa, less than about 30 kDa, less than about 25 kDa).

In some embodiments, the Fc domain monomer includes at least 200 amino acid residues (e.g., at least 210, at least 220, at least 230, at least 240, at least 250, at least 260, at least 270, at least 280, at least 290, or at least 300 amino residues). In some embodiments, the Fc domain monomer is at least 20 kDa (e.g., at least 25 kDa, at least 30 kDa, or at least 35 kDa).

In some embodiments, the Fc domain monomer includes 200 to 400 amino acid residues (e.g., 200 to 250, 250 to 300, 300 to 350, 350 to 400, 200 to 300, 250 to 350, or 300 to 400 amino acid residues). In some embodiments, the Fc domain monomer is 20 to 40 kDa (e.g., 20 to 25 kDa, 25 to 30 kDa, 35 to 40 kDa, 20 to 30 kDa, 25 to 35 kDa, or 30 to 40 KDa).

In some embodiments, the Fc domain monomer includes an amino acid sequence at least 90% identical (e.g., at least 95%, at least 98%) to the sequence of any one of SEQ ID Nos: 1-112 and 115-120, or a region thereof. In some embodiments, the Fc domain monomer includes the amino acid sequence of any one of SEQ ID NOs: 1-112 and 115-120, or a region thereof.

In some embodiments, the Fc domain monomer includes a region of any one of SEQ ID NOs: 1-112 and 115-120, wherein the region includes positions 220, 252, 254, and 256. In some embodiments, the region includes at least 40 amino acid residues, at least 50 amino acid residues, at least 60 amino acid residues, at least 70 amino acids residues, at least 80 amino acids residues, at least 90 amino acid residues, at least 100 amino acid residues, at least 110 amino acid residues, at least 120 amino residues, at least 130 amino acid residues, at least 140 amino acid residues, at least 150 amino acid residues, at least 160 amino acid residues, at least 170 amino acid residues, at least 180 amino acid residues, at least 190 amino acid residues, or at least 200 amino acid residues.

### Activation of Immune Cells

Fc-gamma receptors (FcγRs) bind the Fc portion of immunoglobulin G (IgG) and play important roles in immune activation and regulation. For example, the IgG Fc domains in immune complexes (Ics) engage FcγRs with high avidity, thus triggering signaling cascades that regulate immune cell activation. The human FcγR family contains several activating receptors (FcγRI, FcγRIIa, FcγRIIc, FcγRIIIa, and FcγRIIIb) and one inhibitory receptor (FcγRIIb). FcγR signaling is mediated by intracellular domains that contain immune tyrosine activating motifs (ITAMs) for activating FcγRs and immune tyrosine inhibitory motifs (ITIM) for inhibitory receptor FcγRIIb. In some embodiments, FcγR binding by Fc domains results in ITAM phosphorylation by Src family kinases; this activates Syk family kinases and induces downstream signaling networks, which include PI3K and Ras pathways.

In the fusion proteins or conjugates described herein, the Fc domain portion of the fusion protein or conjugate bind to FcγRs (e.g., FcRn, FcγRI, FcγRIIa, FcγRIIc, FcγRIIIa, and FcγRIIIb) on immune cells and activate phagocytosis and effector functions, such as antibody-dependent cell-mediated cytotoxicity (ADCC). Examples of immune cells that may be activated by the conjugates described herein include, but are not limited to, macrophages, neutrophils, eosinophils, basophils, lymphocytes, follicular dendritic cells, natural killer cells, and mast cells.

### Tissue Distribution

After a therapeutic enters the systemic circulation, it is distributed to the body's tissues. Distribution is generally uneven because of different in blood perfusion, tissue binding, regional pH, and permeability of cell membranes. The entry rate of a drug into a tissue depends on the rate of blood flow to the tissue, tissue mass, and partition characteristics between blood and tissue. Distribution equilibrium (when the entry and exit rates are the same) between blood and tissue is reached more rapidly in richly vascularized areas unless diffusion across cell membranes is the rate-limiting step. The size, shape, charge, target binding, FcRn and target binding mechanisms, route of administration, and formulation affect tissue distribution.

In some instances, the fusion proteins described herein may be optimized to distribute to lung tissue. In some instances, the fusion proteins have a concentration ratio of distribution in epithelial lining fluid of at least 30% the concentration of the fusion protein in plasma within 2 hours after administration. In certain embodiments, ratio of the concentration is at least 45% within 2 hours after administration. In some embodiments, the ratio of concentration is at least 55% within 2 hours after administration. In particular, the ratio of concentration is at least 60% within 2 hours after administration.

### III. Linkers

A linker refers to a linkage or connection between two or more components in a conjugate described herein (e.g., between two CD73 inhibitors in a conjugate described herein, between a CD73 inhibitor and an Fc domain in a conjugate described herein, and between a dimer of two CD73 inhibitors and an Fc domain in a conjugate described herein).

### Linkers in Conjugates Having an Fc Domain Covalently Linked to Monomers of CD73 Inhibitors

In a conjugate containing an Fc domain monomer or an Fc domain covalently linked to one or more monomers of CD73 inhibitors as described herein, a linker in the conjugate may be a divalent structure having two arms. One arm in a divalent linker may be attached to the monomer of CD73 inhibitor and the other arm may be attached to the Fc domain monomer or an Fc domain. In some embodiments, the one or more monomers of CD73 inhibitors in the conjugates described herein may each be, independently, connected to an atom in the Fc domain monomer or an Fc domain.

In some embodiments, a linker is described by formula:

J¹-(Q¹)_{g}-(T¹)ₕ-(Q²)ᵢ-(T²)ⱼ-(Q³)ₖ-(T³)ₗ-(Q⁴)ₘ-(T⁴)ₙ-(Q⁵)ₒ-J²

wherein J¹ is a bond attached to A₁; J² is a bond attached to E or is a functional group capable of reacting with a functional group conjugated to E (e.g., maleimide and cysteine, amine and activated carboxylic acid (e.g., carboxylic acid activated by tetrafluorophenyl or trifluorophenol), thiol and maleimide, activated sulfonic acid and amine, isocyanate and amine, azide and alkyne, and alkene and tetrazine); each of Q¹, Q², Q³, Q⁴ and Q⁵ is, independently, optionally substituted C1-C40 alkylene, optionally substituted C1-C40 heteroalkylene, optionally substituted C1-C40 alkoxylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C₂-C₁₅ heteroarylene; each of T¹, T², T³, T⁴ is, independently, O, S, NRⁱ, P, carbonyl, thiocarbonyl, sulfonyl, phosphate, phosphoryl, imino, or oximo; R¹ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C2-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and each of g, h, i, j, k, l, m, n, and o is, independently, 0, 1, or 2. In some embodiments, each of g, h, i, j, k, l, m, n, and o is, independently, 0 or 1.

In some embodiments, optionally substituted includes substitution with a polyethylene glycol (PEG). A PEG has a repeating unit structure (—CH₂CH₂O—)ₙ, wherein n is an integer from 2 to 100. A polyethylene glycol may be selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀).

In some embodiments, J² may have two points of attachment to the Fc domain monomer or Fc domain (e.g., two J²).

### Linkers in Conjugates Having an Fc Domain Covalently Linked to Dimers of CD73 Inhibitors

In a conjugate containing an Fc domain monomer or an Fc domain covalently linked to one or more dimers of CD73 inhibitors as described herein, a linker in the conjugate may be a branched structure. As described further herein, a linker in a conjugate described herein may be a multivalent structure, e.g., a divalent or trivalent structure having two or three arms, respectively. In some embodiments when the linker has three arms, two of the arms may be attached to the first and second CD73 inhibitors and the third arm may be attached to the Fc domain monomer or an Fc domain. In some embodiments when the linker has two arms, one arm may be attached to an Fc domain and the other arm may be attached to one of the two CD73 inhibitors. In other embodiments, a linker with two arms may be used to attach the two CD73 inhibitors on a conjugate containing an Fc domain covalently linked to one or more dimers of CD73 inhibitors.

In some embodiments, a linker in a conjugate having an Fc domain covalently linked to one or more dimers of CD73 inhibitors is described by formula (D-L-1):

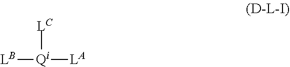

wherein L^{A} is described by formula G^{A1}-(Z^{A1})_{g1}—(Y^{A1})ₕ₁—(Z^{A2})ᵢ₁—(Y^{A2})ⱼ₁—(Z^{A3})ₖ₁—(Y^{A3})ₗ₁—(Z^{A4})ₘ₁—(Y^{A4})ₙ₁—(Z^{A5})o₁-G^{A2}; L^{B} is described by formula G^{B1}-(Z^{B1})_{g2}—(Y^{B1})ₕ₂—(Z^{B2})ᵢ₂—(Y^{B2})ⱼ₂—(Z^{B3})ₖ₂—(Y^{B3})ₗ₂—(Z^{B4})ₘ₂—(Y^{B4})ₙ₂—(Z^{B5})o₂-G^{B2}; L^{C} is described by formula G^{C1}-(Z^{C1})_{g3}-(Y^{C1})ₕ₃—(Z^{C2})ᵢ₃—(Y^{C2})ⱼ₃—(Z^{C3})ₖ₃—(Y^{C3})ₗ₃—(Z^{C4})ₘ₃—(Y^{C4})ₙ₃—(Z^{C5})o₃-G^{C2}; G^{A1} is a bond attached to Q¹ in formula (D-L-1); G^{A2} is a bond attached to the first CD73 inhibitor (e.g., A₁); G^{B1} is a bond attached to Q¹ in formula (D-L-1); G^{B2} is a bond attached to the second CD73 inhibitor (e.g., A₂); G^{C1} is a bond attached to Q¹ in formula (D-L-1); G^{C2} is a bond attached to an Fc domain monomer or an Fc domain or is a functional group capable of reacting with a functional group conjugated to E (e.g., maleimide and cysteine, amine and activated carboxylic acid, thiol and maleimide, activated sulfonic acid and amine, isocyanate and amine, azide and alkyne, and alkene and tetrazine); each of Z^{A1}, Z^{A2}, Z^{A3}, Z^{A4}, Z^{A5}, Z^{B1}, Z^{B2}, Z^{B3}, Z^{B4}, Z^{B5}, Z^{C1} Z^{C2}, Z^{C3}, Z^{C4}, and Z^{C5} is, independently, optionally substituted C₁-C₄₀ alkylene, optionally substituted C1-C40 heteroalkylene, optionally substituted C1-C40 alkoxylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene; each of Y^{A1}, Y^{A2}, Y^{A3}, Y^{A4}, Y^{B1}, Y^{B2}, Y^{B3}, Y^{B4}, Y^{C1}, Y^{C2}, Y^{C3} and Y^{C4} is, independently, O, S, NRⁱ, P, carbonyl, thiocarbonyl, sulfonyl, phosphate, phosphoryl, imino, or oximo; Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C2-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; each of g1, h1, i1, j1, k1, l1, m1, n1, o1, g2, h2, i2, j2, k2, l2, m2, n2, o2, g3, h3, i3, j3, k3, l3, m3, n3, and o3 is, independently, 0 or 1; Q is a nitrogen atom, optionally substituted C₁-C₂₀ alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C2-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene.

In some embodiments, optionally substituted includes substitution with a PEG. A PEG has a repeating unit structure (—CH₂CH₂O—)ₙ, wherein n is an integer from 2 to 100. A polyethylene glycol may be selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀).

In some embodiments, L^{C} may have two points of attachment to the Fc domain (e.g., two G^{C2}) In some embodiments, L includes a polyethylene glycol (PEG) linker. A PEG linker includes a linker having the repeating unit structure (—CH₂CH₂O—)ₙ, where n is an integer from 2 to 100. A polyethylene glycol linker may covalently join a CD73 inhibitor and E (e.g., in a conjugate of formula (M-I)). A polyethylene glycol linker may covalently join a first CD73 inhibitor and a second CD73 inhibitor (e.g., in a conjugate of formula (D-I)). A polyethylene glycol linker may covalently join a CD73 inhibitor dimer and E (e.g., in a conjugate of formula (D-I)). A polyethylene glycol linker may be selected from any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀). In some embodiments, L^{C} includes a PEG linker, where L^{C} is covalently attached to each of Qⁱ and E.

### Linkers

In some embodiments, linker provides space, rigidity, and/or flexibility between the CD73 inhibitors and the Fc domain monomer or an Fc domain in the conjugates described here or between two CD73 inhibitors in the conjugates described herein. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation. In some embodiments, a linker (e.g., L as shown in formula (D-I) or (M-I)) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker (L) includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker (L) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). The “backbone” of a linker refers to the atoms in the linker that together form the shortest path from one part of the conjugate to another part of the conjugate. The atoms in the backbone of the linker are directly involved in linking one part of the conjugate to another part of the conjugate. For example, hydrogen atoms attached to carbons in the backbone of the linker are not considered as directly involved in linking one part of the conjugate to another part of the conjugate.

Molecules that may be used to make linkers (L) include at least two functional groups, e.g., two carboxylic acid groups. In some embodiments of a trivalent linker, two arms of a linker may contain two dicarboxylic acids, in which the first carboxylic acid may form a covalent linkage with the first CD73 inhibitor in the conjugate and the second carboxylic acid may form a covalent linkage with the second CD73 inhibitor in the conjugate, and the third arm of the linker may for a covalent linkage (e.g., a C—O bond) with an Fc domain monomer or an Fc domain in the conjugate. In some embodiments of a divalent linker, the divalent linker may contain two carboxylic acids, in which the first carboxylic acid may form a covalent linkage with one component (e.g., a CD73 inhibitor) in the conjugate and the second carboxylic acid may form a covalent linkage (e.g., a C—S bond or a C—N bond) with another component (e.g., an Fc domain monomer or an Fc domain) in the conjugate.

In some embodiments, dicarboxylic acid molecules may be used as linkers (e.g., a dicarboxylic acid linker). For example, in a conjugate containing an Fc domain monomer an Fc domain covalently linked to one or more dimers of CD73 inhibitors, the first carboxylic acid in a dicarboxylic acid molecule may form a covalent linkage with a hydroxyl or amine group of the first CD73 inhibitor and the second carboxylic acid may form a covalent linkage with a hydroxyl or amine group of the second CD73 inhibitor.

In some embodiments, dicarboxylic acid molecules, such as the ones described herein, may be further functionalized to contain one or more additional functional groups. Dicarboxylic acids may be further functionalized, for example, to provide an attachment point to an Fc domain monomer or an Fc domain (e.g., by way of a linker, such as a PEG linker).

In some embodiments, when the CD73 inhibitor is attached to Fc domain monomer or an Fc domain, the linker may include a moiety including a carboxylic acid moiety and an amino moiety that are spaced by from 1 to 25 atoms.

In some embodiments, a linker may include a diamino moiety, such as the ones described herein, may be further functionalized to contain one or more additional functional groups. Such diamino linker may be further functionalized, for example, to provide an attachment point to an Fc domain monomer or an Fc domain (e.g., by way of a linker, such as a PEG linker).

In some embodiments, a molecule containing an azide group may be used to form a linker, in which the azide group may undergo cycloaddition with an alkyne to form a 1,2,3-triazole linkage. In some embodiments, a molecule containing an alkyne group may be used to form a linker, in which the alkyne group may undergo cycloaddition with an azide to form a 1,2,3-triazole linkage. In some embodiments, a molecule containing a maleimide group may be used to form a linker, in which the maleimide group may react with a cysteine to form a C—S linkage. In some embodiments, a molecule containing one or more haloalkyl groups may be used to form a linker, in which the haloalkyl group may form a covalent linkage, e.g., C—N and C—O linkages, with a CD73 inhibitor.

In some embodiments, a linker (L) may include a synthetic group derived from, e.g., a synthetic polymer (e.g., a polyethylene glycol (PEG) polymer). In some embodiments, a linker may include one or more amino acid residues. In some embodiments, a linker may be an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker (L) may include one or more optionally substituted C1-C40 alkylene, optionally substituted C1-C40 heteroalkylene (e.g., a PEG unit), optionally substituted C1-C40 alkoxylene, optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C2-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C2-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

### Conjugation Chemistries

CD73 inhibitor monomers or dimers may be conjugated to an Fc domain monomer or an Fc domain, e.g., by way of a linker, by any standard conjugation chemistries known to those of skill in the art. The following conjugation chemistries are specifically contemplated, e.g., for conjugation of a PEG linker (e.g., a functionalized PEG linker) to an Fc domain monomer or an Fc domain.

Covalent conjugation of two or more components in a conjugate using a linker may be accomplished using well-known organic chemical synthesis techniques and methods. Complementary functional groups on two components may react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine. Site-specific conjugation to a polypeptide may accomplished using techniques known in the art. Exemplary techniques for site-specific conjugation of a small molecule to an Fc domain are provided in Agarwall. P., et al. Bioconjugate Chem. 26:176-192 (2015).

Other examples of functional groups capable of reacting with amino groups include, e.g., alkylating and acylating agents. Representative alkylating agents include: (i) an α-haloacetyl group, e.g., XCH₂CO— (where X=Br, Cl, or I); (ii) a N-maleimide group, which may react with amino groups either through a Michael type reaction or through acylation by addition to the ring carbonyl group; (iii) an aryl halide, e.g., a nitrohaloaromatic group; (iv) an alkyl halide; (v) an aldehyde or ketone capable of Schiff's base formation with amino groups; (vi) an epoxide, e.g., an epichlorohydrin and a bisoxirane, which may react with amino, sulfhydryl, or phenolic hydroxyl groups; (vii) a chlorine-containing of s-triazine, which is reactive towards nucleophiles such as amino, sulfhydryl, and hydroxyl groups; (viii) an aziridine, which is reactive towards nucleophiles such as amino groups by ring opening; (ix) a squaric acid diethyl ester; and (x) an α-haloalkyl ether.

Examples of amino-reactive acylating groups include, e.g., (i) an isocyanate and an isothiocyanate; (ii) a sulfonyl chloride; (iii) an acid halide; (iv) an active ester, e.g., a nitrophenylester or N-hydroxysuccinimidyl ester; (v) an acid anhydride, e.g., a mixed, symmetrical, or N-carboxyanhydride; (vi) an acylazide; and (vii) an imidoester. Aldehydes and ketones may be reacted with amines to form Schiff's bases, which may be stabilized through reductive amination.

It will be appreciated that certain functional groups may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as a -haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

In some embodiments, a linker of the disclosure (e.g., L, such as L^{C} of D-L-I), is conjugated (e.g., by any of the methods described herein) to E (e.g., an Fc domain). In preferred embodiments of the disclosure, the linker is conjugated by way of: (a) a thiourea linkage (i.e., —NH(C═S)NH—) to a lysine of E; (b) a carbamate linkage (i.e., —NH(C═O)—O) to a lysine of E; (c) an amine linkage by reductive amination (i.e., —NHCH₂) between a lysine and E; (d) an amide (i.e., —NH—(C═O)CH₂) to a lysine of E; I a cysteine-maleimide conjugate between a maleimide of the linker to a cysteine of E; (f) an amine linkage by reductive amination (i.e., —NHCH₂) between the linker and a carbohydrate of E (e.g., a glycosyl group of an Fc domain monomer or an Fc domain); (g) a rebridged cysteine conjugate, wherein the linker is conjugated to two cysteines of E; (h) an oxime linkage between the linker and a carbohydrate of E (e.g., a glycosyl group of an Fc domain monomer or an Fc domain); (i) an oxime linkage between the linker and an amino acid residue of E; (j) an azido linkage between the linker and E; (k) direct acylation of a linker to E; or (I) a thioether linkage between the linker and E.

In some embodiments, a linker is conjugated to E, wherein the linkage includes the structure —NH(C═NH)X—, wherein X is O, HN, or a bond. In some embodiments, a linker is conjugated to E, wherein the linkage between the remainder of the linker and E includes the structure —NH(C═O)NH—.

In some embodiments, a linker is conjugated to E, wherein the linkage includes the structure —R₉OR₉C(═O)NH—, wherein R₉ is H, optionally substituted C₁-C₂₀ alkyl, optionally substituted C₃-C₂₀ cycloalkyl, optionally substituted C₂-C₂₀ heterocycloalkyl; optionally substituted C₅-C₁₅ aryl, or optionally substituted C₂-C₁₅ heteroaryl. In some embodiments, the linker is conjugated to E, wherein the linkage between the remainder of the linker and E includes the structure —CH₂OCH₂C(═O)NH—.

Exemplary linking strategies (e.g., methods for linking a monomer or a dimer of a CD73 inhibitor to E, such as, by way of a linker) are further described in the Examples.

In some embodiments, a linker (e.g., an active ester, e.g., a nitrophenylester or N-hydroxysuccinimidyl ester, or derivatives thereof (e.g., a functionalized PEG linker (e.g., azido-PEG₂-PEG₄₀-NHS ester), is conjugated to E, with a T of (e.g., drug-antibody ratio or DAR) of between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In these instances, the E-(PEG₂-PEG₄₀)-azide can react with an Int having a terminal alkyne linker (e.g., L, such as L^{C} of D-L-I) through click conjugation. During click conjugation, the copper-catalyzed reaction of the azide (e.g., the Fc-(PEG₂-PEG₄₀)-azide) with the alkyne (e.g., the Int having a terminal alkyne linker (e.g., L, such as L^{C} of D-L-I) forming a 5-membered heteroatom ring. In some embodiments, the linker conjugated to E is a terminal alkyne and is conjugated to an Int having a terminal azide. Exemplary preparations of preparations of E-(PEG₂-PEG₄₀)-azide are described in the Examples. One of skill in the art would readily understand the final product from a click chemistry conjugation.

Exemplary linking strategies are further depicted herein.

### IV. Methods of Treatment

This disclosure provides uses of conjugates and pharmaceutical compositions described herein in the treatment of disorders associated with dysregulation or overexpression of CD73 (e.g., cancer, fibrosis, or a viral infection).

### Cancer

The conjugates and pharmaceutical compositions described herein can be used to treat a cancer in a subject. In some embodiments, the cancer overexpresses or is known to overexpress CD73 relative to a non-cancerous cell of the same tissue type. In some embodiments, the subject has been determined to have a cancer that overexpresses CD73 relative to a non-cancerous cell of the same tissue type. In some embodiments, the method further comprises a step of determining whether the cancer overexpresses CD73 relative to a non-cancerous cell of the same tissue type and administering the conjugate only if the cancer overexpresses CD73.

In some embodiments, the cancer is selected from lung cancer, optionally non-small cell lung cancer or small-cell lung cancer; head and neck cancer, optionally squamous cell carcinoma; renal cell carcinoma; breast cancer; ovarian cancer; pancreatic cancer; colorectal cancer; urothelial cancer; bile duct cancer; endometrial cancer; melanoma; or esophageal cancer. In some embodiments, the cancer is a solid tumor.

In some embodiments, the method further includes administering to the subject an immune checkpoint inhibitor. In some embodiments, the immune checkpoint inhibitor is an inhibitor of any one of more of the following immune checkpoint targets: CTLA-4, PD-1, PD-L1, LAG-3, B7.1, B7-H3, B7-H4, TIM3, VISTA, CD137, OX-40, CD40, CD27, CCR4, GITR, NKG2D, and KIR. In some embodiments, the immune checkpoint inhibitor is a monoclonal antibody selected from one or more of an anti-PD-1 antibody, an anti-PD-L1 antibody, an anti-CTLA-4 antibody, an anti-LAG3 antibody, an anti-B7.1 antibody, an anti-B7H3 antibody, an anti-B7H4 antibody, an anti-TIM3 antibody, an anti-VISTA antibody, an anti-CD137 antibody, an anti-OX40 antibody, an anti-CD40 antibody, an anti-CD27 antibody, an anti-CCR4 antibody, an anti-GITR antibody, an anti-NKG2D antibody, and an anti-KIR antibody. In some embodiments, the immune checkpoint inhibitor is a PD-1 inhibitor. In some embodiments, the PD-1 inhibitor is an anti-PD-1 monoclonal antibody.

Immune checkpoint inhibitors approved or in development include, but are not limited to, YERVOY® (ipilimumab), OPDIVO® (nivolumab), KEYTRUDA® (pembrolizumab), tremelimumab, galiximab, MDX-1106, BMS-936558, MEDI4736, MPDL3280A, MEDI6469, BMS-986016, BMS-663513, PF-05082566, IPH2101, KW-0761, CDX-1127, CP-870, CP-893, GSK2831781, MSB0010718C, MK3475, CT-011, AMP-224, MDX-1105, IMP321, and MG^{A271}, as well as numerous other antibodies or fusion proteins directed to immune checkpoint proteins described herein.

In some embodiments, the method includes administering to said subject (1) a conjugate described herein and (2) an immune checkpoint inhibitor. In some embodiments, the conjugate described herein is administered first, followed by administering of the immune checkpoint inhibitor alone. In some embodiments, the immune checkpoint inhibitor is administered first, followed by administering of the conjugate described herein alone. In some embodiments, the conjugate described herein and the immune checkpoint inhibitor are administered substantially simultaneously (e.g., in the same pharmaceutical composition or in separate pharmaceutical compositions). In some embodiments, when a conjugate described herein and an immune checkpoint inhibitor are administered together (e.g., substantially simultaneously in the same or separate pharmaceutical compositions, or separately in the same treatment regimen), tumor growth suppression of each of the conjugate and the immune checkpoint inhibitor may be greater (e.g., occur at a lower concentration) than inhibition of tumor growth suppression of each of the conjugate and the immune checkpoint inhibitor when each is used alone in a treatment regimen.

### Viral Infections

The conjugates and pharmaceutical compositions described herein can be used to treat viral infections in a subject. The conjugates and pharmaceutical compositions described herein can also be used to prevent viral infections in a subject susceptible to viral infection or at increased risk of contracting a viral infection (e.g., a subject that is hospitalized, immunocompromised, who is preparing for surgery, who recently underwent surgery, or who is taking a medication that affects the immune system, such as a chemotherapy of radiation).

In some embodiments, the viral infection is a betacoronavirus infection. In some embodiments, the betacoronavirus is SARS-CoV-2. In some embodiments, the SARS-CoV-2 is an Alpha, Delta, or Omicron variant. In some embodiments, the SARS-CoV-2 is an Omicron variant. In some embodiments, the Omicron variant is a BA.1, BA.2, BA.3, BA.4, or BA.5 lineage.

In some embodiments, the method further includes administering to the subject an antiviral agent or an antiviral vaccine. In some embodiments, the method includes administering to said subject (1) a conjugate described herein and (2) an antiviral agent or an antiviral vaccine. In some embodiments, the conjugate described herein is administered first, followed by administering of the antiviral agent or antiviral vaccine alone. In some embodiments, the antiviral agent or antiviral vaccine is administered first, followed by administering of the conjugate described herein alone. In some embodiments, the conjugate described herein and the antiviral agent or antiviral vaccine are administered substantially simultaneously (e.g., in the same pharmaceutical composition or in separate pharmaceutical compositions). In some embodiments, when a conjugate described herein and an antiviral agent or antiviral vaccine are administered together (e.g., substantially simultaneously in the same or separate pharmaceutical compositions, or separately in the same treatment regimen), inhibition of viral replication of each of the conjugate and the antiviral agent or antiviral vaccine may be greater (e.g., occur at a lower concentration) than inhibition of viral replication of each of the conjugate and the antiviral agent or antiviral vaccine when each is used alone in a treatment regimen.

### Fibrosis

The conjugates and pharmaceutical compositions described herein can be used to treat or prevent fibrosis in a subject.

In some embodiments, the fibrosis is pulmonary fibrosis, dermal fibrosis, renal fibrosis, hepatic fibrosis, cardiac fibrosis, or systemic sclerosis. In some embodiments, the fibrosis is pulmonary fibrosis. In some embodiments, the pulmonary fibrosis associated with a viral infection (e.g., associated with a SARS-CoV-2 infection), drug-induced pulmonary fibrosis, radiation-induced pulmonary fibrosis, hypersensitivity pneumonitis, idiopathic pulmonary fibrosis, non-specific interstitial pneumonia, pneumoconiosis, interstitial lung disease, sarcoidosis, silicosis, or systemic sclerosis.

In some embodiments, the fibrosis is selected from the group consisting of scleroderma, cystic fibrosis, liver cirrhosis, interstitial pulmonary fibrosis, idiopathic pulmonary fibrosis, Dupuytren's contracture, keloids, chronic kidney disease, chronic graft rejection, scarring, wound healing, post-operative adhesions, reactive fibrosis, polymyositis, ANCA vasculitis, Behcet's disease, anti-phospholipid syndrome, relapsing polychondritis, Familial Mediterranean Fever, giant cell arteritis, Graves ophthalmopathy, discoid lupus, pemphigus, bullous pemphigoid, hydradenitis suppuritiva, sarcoidosis, bronchiolitis obliterans, primary sclerosing cholangitis, primary biliary cirrhosis, and organ fibrosis (e.g., dermal fibrosis, lung fibrosis, liver fibrosis, kidney fibrosis, or heart fibrosis). In some embodiments, the fibrosis is scleroderma (e.g., systemic sclerosis, localized scleroderma, or sine scleroderma). In some embodiments, the fibrosis is organ fibrosis (e.g., dermal fibrosis, lung fibrosis, liver fibrosis, kidney fibrosis, or heart fibrosis). In some embodiments, the fibrosis is cystic fibrosis.

Treatment of fibrosis may be assessed by suitable methods known to one of skill in the art including the improvement, amelioration, or slowing the progression of one or more symptoms associated with the particular fibrotic disease being treated.

### V. Pharmaceutical Compositions

A conjugate described herein may be formulated in a pharmaceutical composition for use in the methods described herein. In some embodiments, a conjugate described herein may be formulated in a pharmaceutical composition alone. In some embodiments, a conjugate described herein may be formulated in combination with a second therapeutic agent in a pharmaceutical composition. In some embodiments, a conjugate described herein may be administered in combination with a second therapeutic agent as part of a dosing regimen (e.g., administered sequentially or simultaneously). In some embodiments, the pharmaceutical composition includes a conjugate described herein and pharmaceutically acceptable carriers and excipients.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acid residues such as glycine, glutamine, histidine, and lysine, and carbohydrates such as glucose, mannose, sucrose, and sorbitol.

Examples of other excipients include, but are not limited to, antiadherents, binders, coatings, compression aids, disintegrants, dyes, emollients, emulsifiers, fillers (diluents), film formers or coatings, flavors, fragrances, glidants (flow enhancers), lubricants, sorbents, suspensing or dispersing agents, or sweeteners. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The conjugates herein may have ionizable groups so as to be capable of preparation as pharmaceutically acceptable salts. These salts may be acid addition salts involving inorganic or organic acids or the salts may, in the case of acidic forms of the conjugates herein be prepared from inorganic or organic bases. Frequently, the conjugates are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include, but are not limited to, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include, but are not limited to, sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Depending on the route of administration and the dosage, a conjugate herein or a pharmaceutical composition thereof used in the methods described herein will be formulated into suitable pharmaceutical compositions to permit facile delivery. A conjugate or a pharmaceutical composition thereof may be formulated to be administered intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gel cap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). Depending on the route of administration, a conjugate herein or a pharmaceutical composition thereof may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

A conjugate described herein may be formulated in a variety of ways that are known in the art. For use as treatment of human and animal subjects, a conjugate described herein can be formulated as pharmaceutical or veterinary compositions. Depending on the subject (e.g., a human) to be treated, the mode of administration, and the type of treatment desired, e.g., prophylaxis or therapy, a conjugate described herein is formulated in ways consonant with these parameters. A summary of such techniques is found in Remington: The Science and Practice of Pharmacy, 22nd Edition, Lippincott Williams & Wilkins (2012); and Encyclopedia of Pharmaceutical Technology, 4th Edition, J. Swarbrick and J. C. Boylan, Marcel Dekker, New York (2013), each of which is incorporated herein by reference.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, and preservatives. The conjugates can be administered also in liposomal compositions or as microemulsions. Systemic administration may also include relatively noninvasive methods such as the use of suppositories, transdermal patches, transmucosal delivery and intranasal administration. Oral administration is also suitable for conjugates herein. Suitable forms include syrups, capsules, and tablets, as is understood in the art.

The pharmaceutical compositions can be administered parenterally in the form of an injectable formulation. Pharmaceutical compositions for injection can be formulated using a sterile solution or any pharmaceutically acceptable liquid as a vehicle. Formulations may be prepared as solid forms suitable for solution or suspension in liquid prior to injection or as emulsions. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Such injectable compositions may also contain amounts of nontoxic auxiliary substances such as wetting or emulsifying agents, pH buffering agents, such as sodium acetate and sorbitan monolaurate. Formulation methods are known in the art, see e.g., Pharmaceutical Preformulation and Formulation, 2nd Edition, M. Gibson, Taylor & Francis Group, CRC Press (2009).

The pharmaceutical compositions can be prepared in the form of an oral formulation. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Other pharmaceutically acceptable excipients for oral formulations include, but are not limited to, colorants, flavoring agents, plasticizers, humectants, and buffering agents. Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release of a conjugate described herein or a pharmaceutical composition thereof can be achieved by appropriate coating of a tablet, capsule, pellet, or granulate formulation of the conjugate, or by incorporating the conjugate into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The pharmaceutical composition may be formed in a unit dose form as needed. The amount of active component, e.g., a conjugate described herein, included in the pharmaceutical compositions are such that a suitable dose within the designated range is provided (e.g., a dose within the range of 0.01-100 mg/kg of body weight).

### VI. Routes of Administration and Dosages

In any of the methods described herein, conjugates herein may be administered by any appropriate route for treating or protecting against a disorder described herein (e.g., a cancer, viral infection, or fibrotic condition). Conjugates described herein may be administered to humans, domestic pets, livestock, or other animals with a pharmaceutically acceptable diluent, carrier, or excipient. In some embodiments, administering includes administration of any of the conjugates described herein or compositions intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gel cap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). In some embodiments, if a second therapeutic agent is also administered in addition to a conjugate described herein, the second therapeutic agent or a pharmaceutical composition thereof may also be administered in any of the routes of administration described herein.

The dosage of a conjugate described herein or pharmaceutical compositions thereof depends on factors including the route of administration, the disease to be treated, and physical characteristics, e.g., age, weight, general health, of the subject. Typically, the amount of the conjugate or the pharmaceutical composition thereof contained within a single dose may be an amount that effectively prevents, delays, or treats the disorder without inducing significant toxicity. A pharmaceutical composition may include a dosage of a conjugate described herein ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 1 to about 30 mg/kg. In some embodiments, when a conjugate described herein and a second therapeutic agent are administered in combination (e.g., substantially simultaneously in the same or separate pharmaceutical compositions, or separately in the same treatment regimen), the dosage needed of the conjugate described herein may be lower than the dosage needed of the conjugate if the conjugate was used alone in a treatment regimen.

A conjugate described herein or a pharmaceutical composition thereof may be administered to a subject in need thereof, for example, one or more times (e.g., 1-10 times or more; 1, 2, 3, 4, 5, 6, 7, 8, 9, or 10 times) daily, weekly, monthly, biannually, annually, or as medically necessary. Dosages may be provided in either a single or multiple dosage regimens. The timing between administrations may decrease as the medical condition improves or increase as the health of the patient declines. The dosage and frequency of administration may be adapted by the physician in accordance with conventional factors such as the extent of the infection and different parameters of the subject.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### General Procedure for Preparation of Fc Constructs

Reverse translations of the amino acids including the protein constructs were synthesized by solid-phase synthesis. The oligonucleotide templates were cloned into pcDNA3.1 (Life Technologies, Carlsbad, CA, USA) at the cloning sites BamHI and XhoI (New England Biolabs, Ipswich, MA, USA) and included signal sequences derived from the human Interleukin-2 or human albumin. The pcDNA3.1 plasmids were transformed into Top10 E. coli cells (LifeTech). DNA was amplified, extracted, and purified using the PURELINK® HiPure Plasmid Filter Maxiprep Kit (LifeTech). The plasmid DNA is delivered, using the EXPIFECTAMINE™ 293 Transfection Kit (LifeTech), into HEK-293 cells per the manufacturer's protocol. Cells were centrifuged, filtered, and the supernatants were purified using MabSelect Sure Resin (GE Healthcare, Chicago, IL, USA). Purified molecules were analyzed using 4-12% Bis Tris SDS PAGE.

### General Procedure for Conjugation of Intermediate (Int) to Fc

A solution of trifluorophenyl ester dissolved in DMF (1 mL), was added to a solution of 50 mg of Fc in PBS at pH 7.4, 19.5 mg/mL) at ambient temperature. The pH of the resulting solution was adjusted to ˜8.5 with borate buffer (300 uL, 1M, pH 8.5) or carbonate buffer (300 uL, 1M, pH 8.5). The homogeneous colorless reaction was rocked gently for 3 h, then submitted for purification according to the general procedure. Maldi TOF analysis of the purified final product gave an average mass from which the DAR is calculated.

### General Procedure for Purification of Conjugates

Protein A, dialysis and SEC: the conjugates were purified using Mabselect PrismA (protein A purification) resin eluted with TBS pH 7.4, followed by dialysis into 150 mM histidine (2×), then 150 mM NaCl pH 8.5 buffer using a Slide-d-lyzer G2 dialysis cassettes (30,000 MWCO), followed by size-exclusion chromatography using TBS pH 7.4 buffer. The final product in TBS (25 mM Tris, 150 mM NaCl) pH 7.4 buffer. Purified material was quantified using a UV visible spectrophotometer (Protein Bradford assay) and concentrated to approximately 10 mg/ml using a centrifugal concentrator (30,000 MWCO).

### Synthesis of Intermediate A

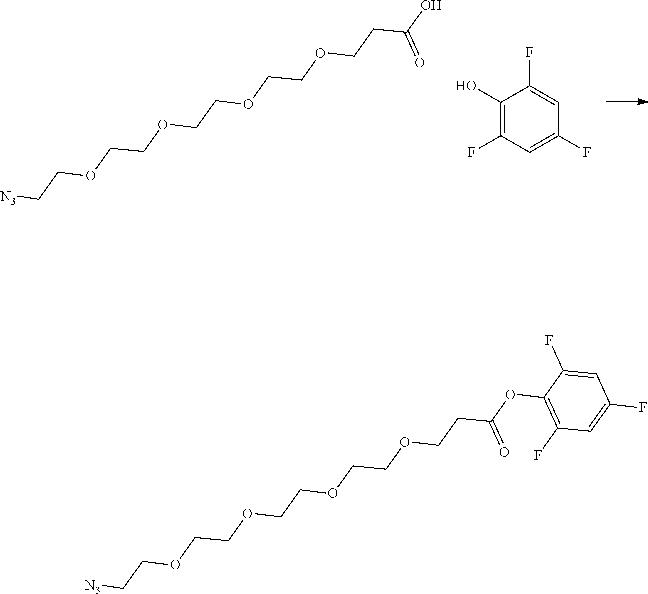 [SMILES: [N-]=[N+]=NCCOCCOCCOCCOCCC(=O)O ; Oc1c(F)cc(F)cc1F ; [N-]=[N+]=NCCOCCOCCOCCOCCC(=O)Oc1c(F)cc(F)cc1F]

EDC (1.6 g, 8.2 mmol) was added, in 4 portions, to a stirring mixture of azido-peg4-carboxylic acid (2 g, 6.9 mmol, and 2,4,6-trifluorophenol (1.2 g, 8.2 mmol) in DCM (20 mL). The mixture was stirred for 2 hours at ambient temperature. The organic phase was washed with DI water (30 mL) and the aqueous phase was back extracted with DCM (2×, 20 mL). The combined organic extracts were dried over sodium sulfate, filtered and concentrated. The crude product mixture was purified by silica gel chromatography (0-80% ethyl acetate in hexanes, 25-minute gradient) to afford the product as a clear oil. Yield 2.15 g, 79%. Ions found by L^{C}/MS [M+Na]⁺=443.8.

### Synthesis of Intermediate B

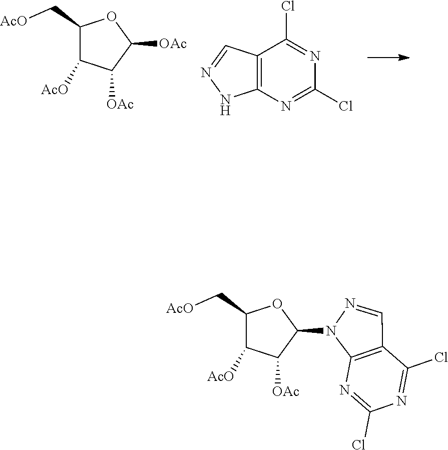 [SMILES: CC(=O)OC[C@H]1O[C@@H](OC(C)=O)[C@H](OC(C)=O)[C@@H]1OC(C)=O ; Clc1nc(Cl)c2cn[nH]c2n1 ; CC(=O)OC[C@H]1O[C@@H](n2ncc3c(Cl)nc(Cl)nc32)[C@H](OC(C)=O)[C@@H]1OC(C)=O]

4,6-Dichloro-1H-pyrazolo[3,4,d]pyrimidine (5 g, 26.5 mmol) and ammonium sulfate (62 mg) were dissolved in 150 mL of hexamethyldisilzane. The mixture was then heated to 130° C. and stirred for 3 hours. The mixture was then concentrated on the rotary evaporator and dried under high vacuum for 12 hours. The solid residue was then taken up in 100 mL of acetonitrile, and the b-D-ribofuranose 1,2,3,5-tetraacetate (9.3 g, 29.1 mmol) was added and the mixture and stirred until all solids were dissolved. The mixture was cooled to 0° C., and TMSOTf (6.2 mL, 34.4 mmol) was added dropwise over a period of 5 minutes. The reaction mixture was gradually warmed to ambient temperature and allowed to stir for 3 hours. The mixture was concentrated and taken up in ethyl acetate (100 mL). The organic extract was washed with saturated sodium bicarbonate, then brine, dried over sodium sulfate, filtered, and concentrated. The crude residue was purified by silica gel column chromatography (hexanes/EtOAc) to provide the desired compound as a white foam. Yield 7.8 g, 66%. Ions found by LCMS: [M+Na]⁺=469.2.

### Synthesis of Intermediate C

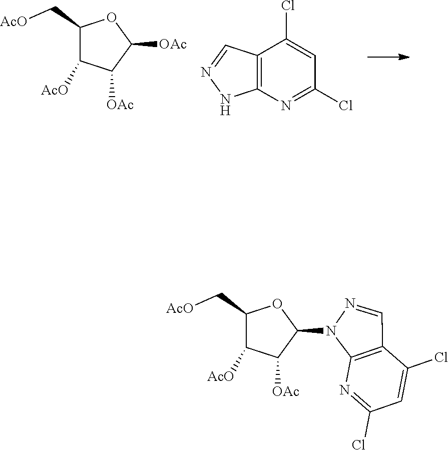 [SMILES: CC(=O)OC[C@H]1O[C@@H](OC(C)=O)[C@H](OC(C)=O)[C@@H]1OC(C)=O ; Clc1cc(Cl)c2cn[nH]c2n1 ; CC(=O)OC[C@H]1O[C@@H](n2ncc3c(Cl)cc(Cl)nc32)[C@H](OC(C)=O)[C@@H]1OC(C)=O]

4,6-Dichloro-1H-pyrazolo[3,4,d]pyridine (2.5 g, 13.3 mmol) and ammonium sulfate (45 mg) were dissolved in 100 mL of hexamethyldisilzane. The mixture was then heated to 130° C. and stirred for 3 hours. The mixture was then concentrated on the rotary evaporator and dried under high vacuum for 12 hours. The solid residue was then taken up in 100 mL of acetonitrile, and the b-D-ribofuranose 1,2,3,5-tetraacetate (3.8 g, 13.3 mmol) was added. The mixture was cooled 0° C., and TMSOTf (3.6 mL, 19.9 mmol) was added dropwise over a period of 5 minutes. The reaction mixture was gradually warmed to ambient temperature and allowed to stir for 3 hours. The mixture was then cooled to 0° C. and saturated sodium bicarbonate was carefully added to neutralize the TMS triflate. The mixture was extracted with ethyl acetate (3×, 40 mL). The organic layer was washed brine, dried over sodium sulfate, filtered, and concentrated. The crude residue was purified by silica gel column chromatography (hexanes/EtOAc) to provide the desired compound as a white foam. Yield 1.85 g, 31%. Ions found by LCMS: [M+H]⁺=446.0.

### Synthesis of Intermediate D

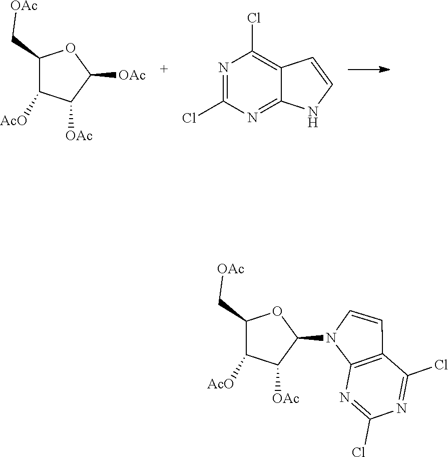 [SMILES: CC(=O)OC[C@H]1O[C@@H](OC(C)=O)[C@H](OC(C)=O)[C@@H]1OC(C)=O ; Clc1nc(Cl)c2cc[nH]c2n1 ; CC(=O)OC[C@H]1O[C@@H](n2ccc3c(Cl)nc(Cl)nc32)[C@H](OC(C)=O)[C@@H]1OC(C)=O]

2,4-Dichloro-7H-pyrrolo[2,3-d]pyrimidine (5 g, 26.6 mmol) and ammonium sulfate (38 mg, 0.291 mmol) were dissolved in 30 mL of hexamethyldisilzane. The mixture was then warmed to reflux and stirred for 3 h. The mixture was then concentrated on the rotary evaporator and dried under high vacuum for 12 hours. The solid residue was then taken up in 100 mL of acetonitrile, and the b-D-ribofuranose 1,2,3,5-tetraacetate (10.2 g, 31.9 mmol) was added. This mixture was cooled 0° C., and TMSOTf (5.28 mL, 29.1 mmol) was added dropwise. The reaction mixture was gradually warmed to room temperature and allowed to stir overnight. The mixture was then concentrated and taken up in ethyl acetate. The organic layer was washed with saturated sodium bicarbonate and brine and dried over sodium sulfate, filtered, and concentrated. The crude residue was purified by silica gel column chromatography (hexanes/EtOAc) to provide the desired compound as a white foam. Yield 2.35 g, 19.8%. Ions found by LCMS: [M+H]⁺=446.2.

### Synthesis of Int-132

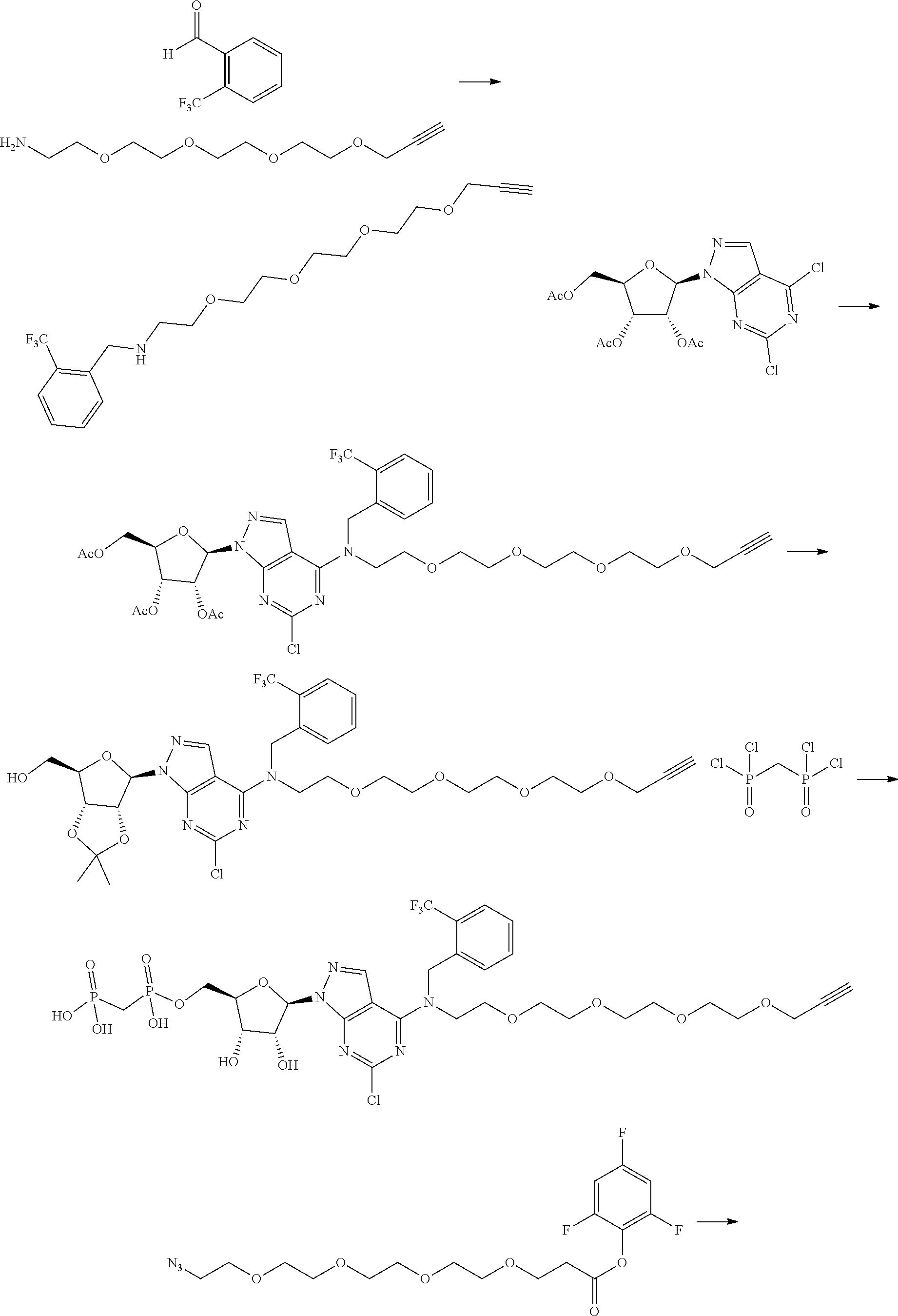

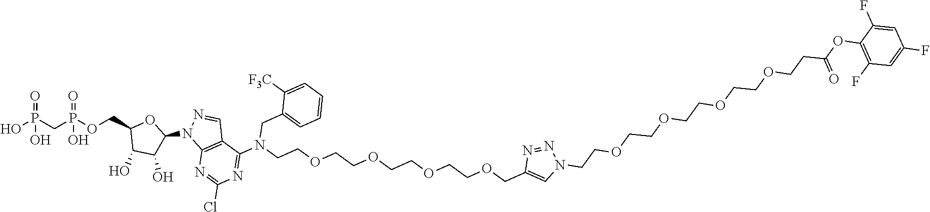 [SMILES: O=C(CCOCCOCCOCCOCCn1cc(COCCOCCOCCOCCN(Cc2ccccc2C(F)(F)F)c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

### Step a

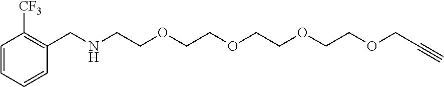 [SMILES: C#CCOCCOCCOCCOCCNCc1ccccc1C(F)(F)F]

Sodium triacetoxy borohydride (412 mg, 1.95 mmol) was added to a stirring mixture of 2-trifluoromethyl benzaldehyde (226 mg, 1.3 mmol) and propargyl-peg4-amine (300 mg, 1.3 mmol) in DCM (25 mL) and the reaction was stirred at ambient temperature for 16 hours. Methanol (3 mL) was added and the mixture was concentrated and purified by silica gel chromatography (0-10% methanol in DCM, 25 min) to afford the product as a clear oil. Yield 73%, 375 mg. Ion(s) found by L^{C}/MS [M+H]+=390.0.

### Step b

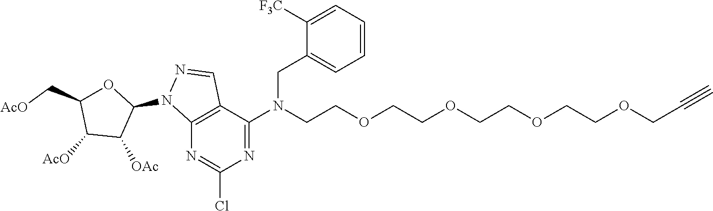 [SMILES: C#CCOCCOCCOCCOCCN(Cc1ccccc1C(F)(F)F)c1nc(Cl)nc2c1cnn2[C@@H]1O[C@H](COC(C)=O)[C@@H](OC(C)=O)[C@H]1OC(C)=O]

Intermediate B (430 mg, 0.96 mmol), the amine product from the previous step (430 mg, 0.96 mmol) and triethylamine (194 mg, 1.93 mmol) were stirred in ethanol (25 mL) at 50° C. for 4 hours. The mixture was cooled to ambient temperature and concentrated. The crude residue was purified by silica gel chromatography (0-10% methanol in DCM, 25 min gradient) to afford the product as a clear oil. Yield 79%, 610 mg. Ion(s) found by L^{C}/MS [M+H]+=800.2.

### Step c

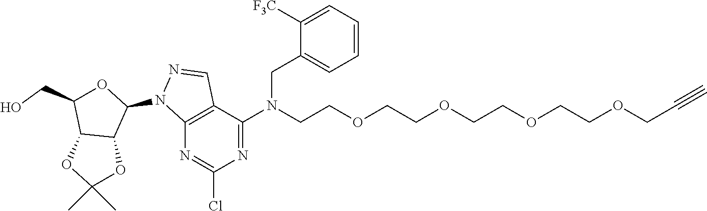 [SMILES: C#CCOCCOCCOCCOCCN(Cc1ccccc1C(F)(F)F)c1nc(Cl)nc2c1cnn2[C@@H]1O[C@H](CO)[C@H]2OC(C)(C)O[C@H]21]

The triacetate product from the previous step (610 mg, 0.76 mmol) and potassium carbonate (30 mg) were stirred in methanol (30 mL) at ambient temperature for 2 hours. The mixture was filtered and neutralized with glacial acetic acid (0.5 mL) and concentrated. The crude residue was purified by silica gel chromatography (0-10% methanol in DCM, 25 min). The triol was taken up in DMF (5 mL) and 2,2 dimethoxy propane (10 mL), p-toluene sulfonic acid hydrate (15 mg) was added and the mixture was stirred at 70° C. for 1 hour. Triethylamine (0.5 mL) was added to the reaction and the mixture was concentrated on the rotary evaporator. The crude residue was purified by silica gel chromatography (0-10% methanol in DCM, 25 min) to afford the product as a clear oil. Yield 89%, 2 steps, 420 mg. Ions found by L^{C}/MS [M+H]+=714.2.

### Step d

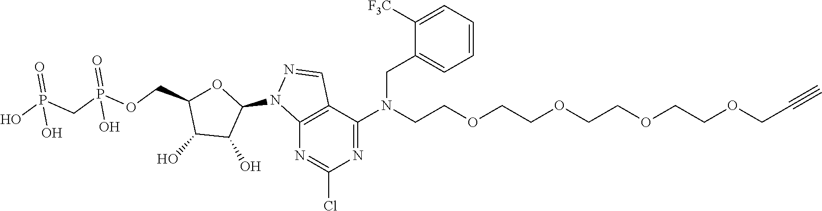 [SMILES: C#CCOCCOCCOCCOCCN(Cc1ccccc1C(F)(F)F)c1nc(Cl)nc2c1cnn2[C@@H]1O[C@H](COP(=O)(O)CP(=O)(O)O)[C@@H](O)[C@H]1O]

The acetonide from the previous step (460 mg, 0.64 mmol) in THE (5 mL) was added dropwise to a mixture of methylene (bis phosphonic dichloride) (633 mg, 2.68 mmol) in THE (5 mL) cooled to 0° C. via an ice bath. When the addition was complete the ice bath was removed and the reaction was stirred for 3 hours at ambient temperature. The mixture was then cooled to 0° C. via an ice bath and 0.5 N aqueous HCl (10 mL) was added. The mixture was stirred at 0° C. for 15 minutes then at ambient temperature for 3 hours (monitored by L^{C}/MS). The solvent was reduced to ˜5 mL on the rotary evaporator and the crude mixture was purified by reversed phase HPLC (5-85% acetonitrile in DI water, 0.1% TFA modifier, 25-minute gradient). The pure fractions were pooled and lyophilized to afford the product as a white, hygroscopic solid. Yield 55%, 295 mg. Ions found by L^{C}/MS [M+H]+=832.2.

### Step e

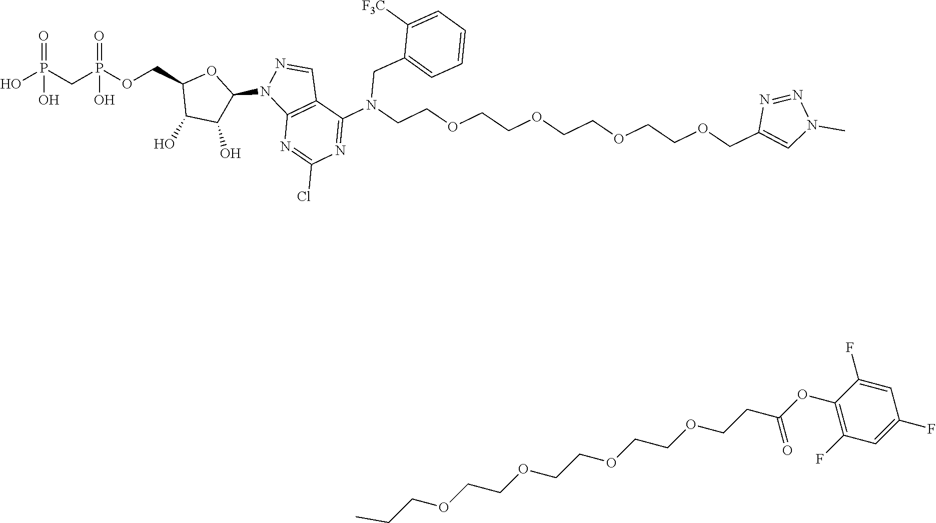

The product from the previous step (70 mg, 0.84 mmol) and intermediate A (35 mg, 0.84 mmol), were dissolved in DMF (1 mL) and cooled to 0° C. via an ice water bath. Copper sulfate (2 mg, 0.013 mmol) was added to a mixture of BTTA (9 mg, 0.021 mmol) and sodium ascorbate (50 mg, 0.25 mmol) in DI water (2 mL) the mixture was gently shaken for 15-20 seconds until the solution was clear. The copper mixture was added the alkyne/azido mixture and the reaction was stirred at 0 C for 10 minutes then at ambient temperature for 20 minutes. The crude reaction mixture was applied directly to reversed phase HPLC (5-85% acetonitrile in DI water, 0.1% TFA modifier, 25-minute gradient). The pure fractions were pooled and lyophilized to afford the product as a white, hygroscopic solid. Yield 43%, 45 mg. Ions found by L^{C}/MS [(M/2)+H]+=627.2.

### Synthesis of Conjugate 70

To a solution of SEQ ID NO: 13 (3.40 mL, 100 mg, 0.0017 mmol) in PBS 7.4 was added Int-132 (21 mg, 0.017 mmol) in DMF (0.200 mL). The pH of the reaction mixture was slowly adjusted to ˜8.5 by the addition of 2 mL of 1M potassium carbonate buffer (pH 9). The reaction was then gently rocked for 4 hours. The reaction was quenched by stirring in a 150 mM His/100 mM ammonium hydroxide buffer (pH 8.5) for 12 hours and then submitted for purification according to the general procedure. Maldi TOF analysis of the purified final product gave an average mass of 62,472 Da (DAR=3.8). Yield: 52 mg, 50%.

### Synthesis of Int-110

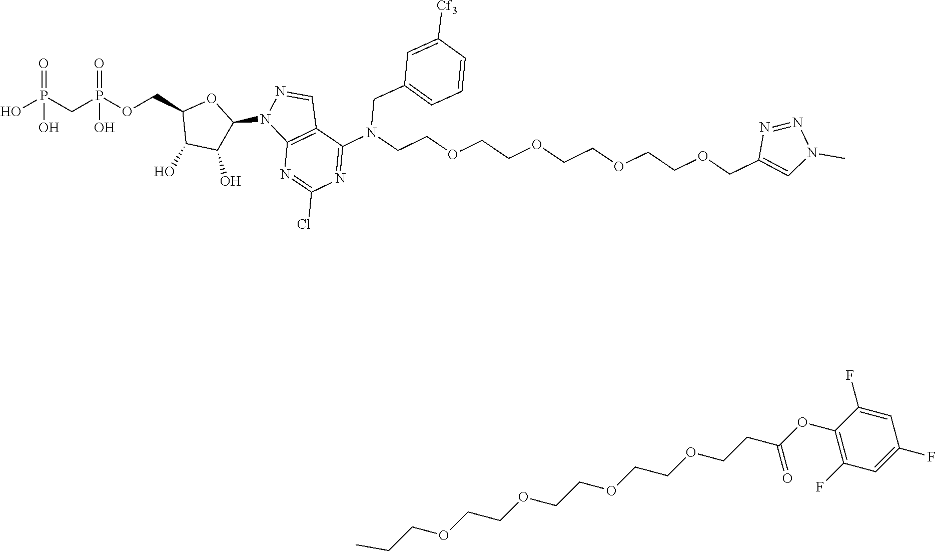

The title compound was prepared as described for Int-132 where 2-trifluoromethyl benzaldehyde was replaced with 3-trifluoromethyl benzaldehyde. Yield 45 mg, 43%. Ions found by L^{C}/MS [(M/2)+H]+=627.2.

### Synthesis of Conjugate 58

The title compound was prepared analogously to Conjugate 70 where the starting material used in Int-132 was replaced by Int-110. Maldi TOF analysis of the purified final product gave an average mass of 63,530 Da (DAR=4.8).

### Synthesis of Int-6

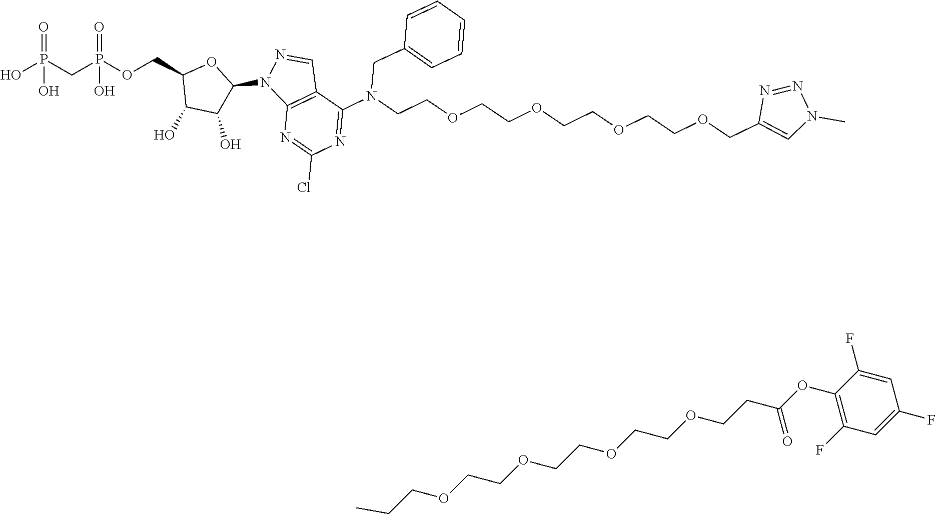

The title compound was prepared as described for Int-132 where 2-trifluoromethyl benzaldehyde was replaced with benzaldehyde. Ions found by L^{C}/MS [(M/2)+H]+=593.2.

### Synthesis of Conjugate 4b

The title compound was prepared analogously to Conjugate 70 where the starting material used in Int-132 was replaced by Int-6. Maldi TOF analysis of the purified final product gave an average mass of 61,147 Da (DAR=2.8).

### Synthesis of Conjugate 4a

To a solution of SEQ ID NO: 17 (5.4 mL, 100 mg, 0.0017 mmol) in PBS 7.4 was added the product described in Int-6 (14 mg, 0.016 mmol) in DMF (0.200 mL). The pH of the reaction mixture was slowly adjusted to ˜8.5 by the addition of 2 mL of 1M potassium carbonate buffer (pH 9). The reaction was then gently rocked for 4 hours then submitted for purification according to the general procedure.

Maldi TOF analysis of the purified final product gave an average mass of 63,505 Da (DAR=5.3). Yield: 76 mg, 75%.

### Synthesis of Int-7

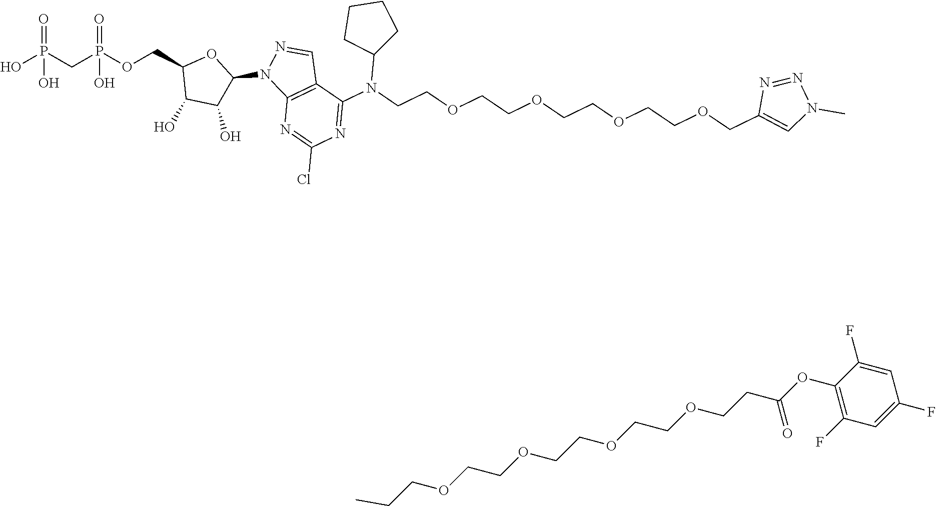

The title compound was prepared as described for Int-132 where 2-trifluoromethyl benzaldehyde was replaced with cyclopentanone. Ion(s) found by L^{C}/MS [(M/2)+H]+=582.2.

### Synthesis of Conjugate 5b

The title compound was prepared analogously to Conjugate 70 where the starting material described in Int-110 was replaced by Int-7. Maldi TOF analysis of the purified final product gave an average mass of 62,224 (DAR=3.9).

### Synthesis of Conjugate 5a

The title compound was prepared analogously to Conjugate 4a where the starting material described in Int-110 was replaced by Int-7. Maldi TOF analysis of the purified final product gave an average mass of 62,404 Da (DAR=4.2).

### Synthesis of Int-74

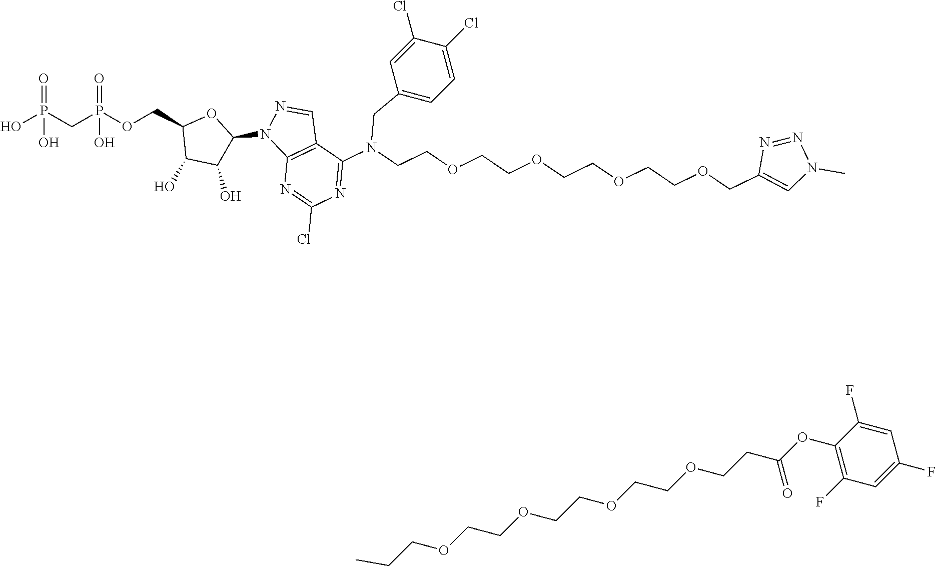

The title compound was prepared as described for Int-132 where 2-trifluoromethyl benzaldehyde was replaced with 3,4-dichloro benzaldehyde. Ions found by L^{C}/MS [(M/2)+H]+=627.2.

### Synthesis of Conjugate 40

The title compound was prepared analogously to Conjugate 70 where the starting material used in Int-110 was replaced by Int-74. Maldi TOF analysis of the purified final product gave an average mass of 66,569 (DAR=7.6).

### Synthesis of Int-75

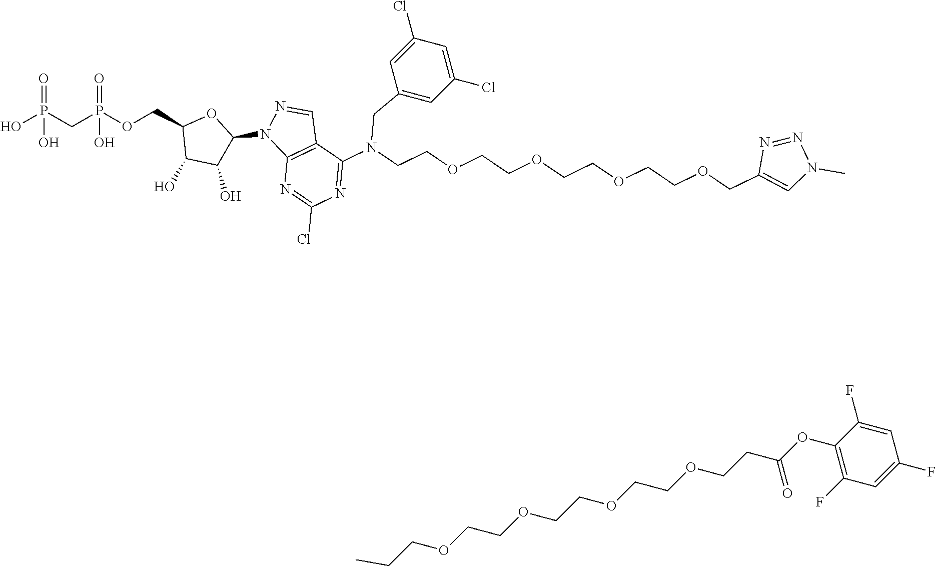

The title compound was prepared as described for Int-132 where 2-trifluoromethyl benzaldehyde was replaced with 3,4-dichloro benzaldehyde. Ions found by L^{C}/MS [(M/2)+H]+=627.2.

### Synthesis of Conjugate 41

The title compound was prepared analogously to Conjugate 70 where the starting material used in Int-110 was replaced by Int-75. Maldi TOF analysis of the purified final product gave an average mass of 63,771 (DAR=5.0).

### Synthesis of Int-111

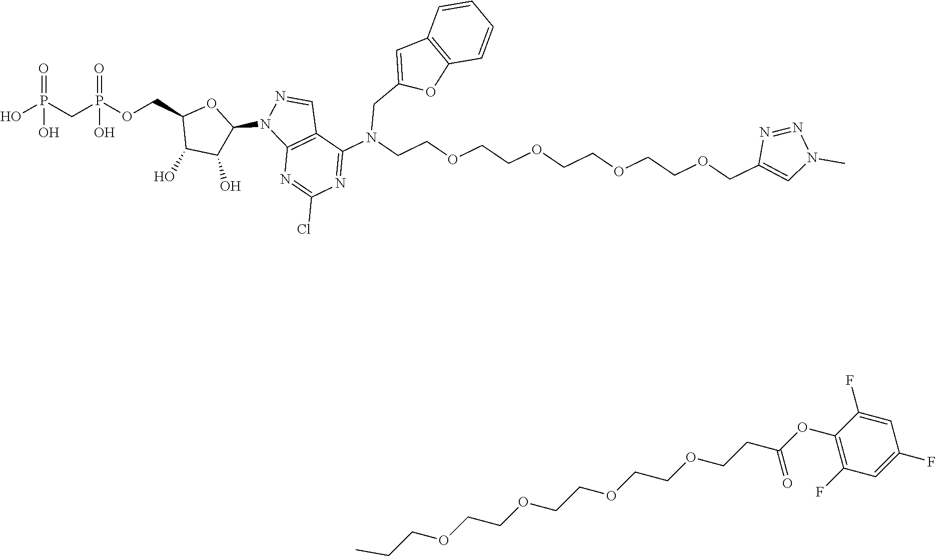

The title compound was prepared as described for Int-132 where 2-trifluoromethyl benzaldehyde was replaced with 1-formylbenzofuran. Ions found by L^{C}/MS [(M/2)+H]+=613.2.

### Synthesis of Conjugate 59

The title compound was prepared analogously to Conjugate 70 where the starting material used in Int-110 was replaced by Int-111. Maldi TOF analysis of the purified final product gave an average mass of 63,411 (DAR=4.7).

### Synthesis of Int-16

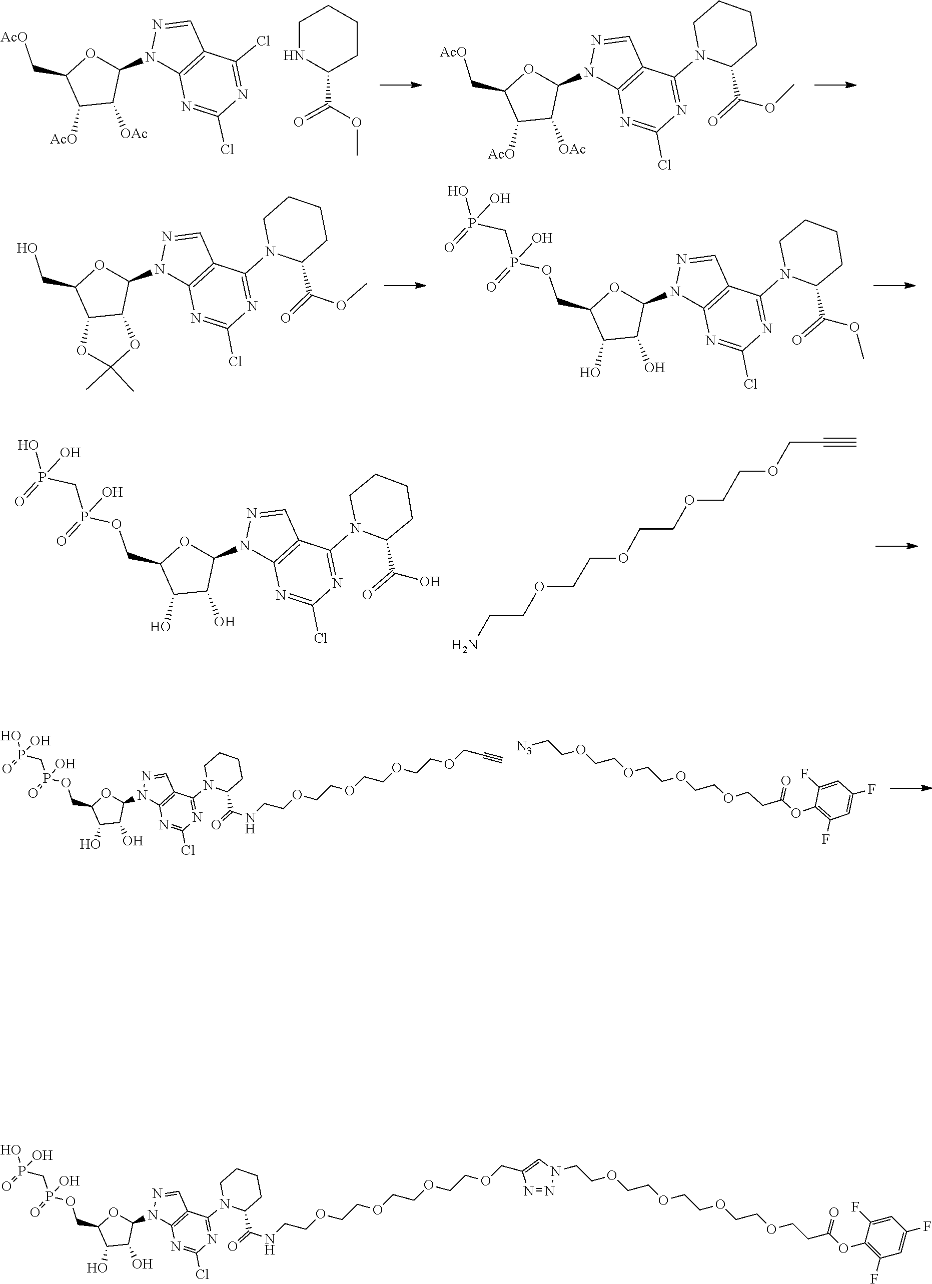

### Step a

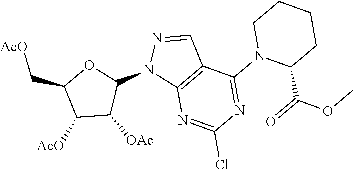

Intermediate B (350 mg, 0.78 mmol), R-methyl piperidine 2-carboxylate (112 mg, 0.78 mmol) and triethylamine (194 mg, 1.93 mmol) were stirred in ethanol (25 mL) at 50° C. for 4 hours. The mixture was cooled to ambient temperature and concentrated. The crude residue was purified by silica gel chromatography (0-10% methanol in DCM, 25 min) to afford the product as a clear oil. Yield 375 mg 85%. Ions found by L^{C}/MS [M+H]+=554.2.

### Step b

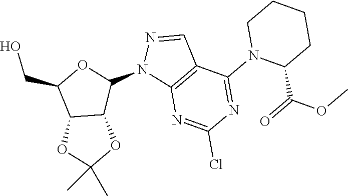

The triacetate product from the previous step (375 mg, 0.68 mmol) and potassium carbonate (30 mg) were stirred in methanol (30 mL) at ambient temperature for 2 hours. The mixture was filtered, neutralized with glacial acetic acid (0.5 mL) and concentrated. The crude residue was purified by silica gel chromatography (0-10% methanol in DCM, 25 min gradient). The triol was taken up in DMF (5 mL) and 2,2 dimethoxy propane (10 mL). p-Toluene sulfonic acid hydrate (15 mg) was added to the reaction and the mixture was stirred at 70° C. for 1 hour. Triethylamine (0.5 mL) was added to the reaction and the mixture was concentrated on the rotary evaporator. The crude residue was purified by silica gel chromatography (0-10% methanol in DCM, 25 min) to afford the product as a clear oil. Yield 175 mg, 54%, 2 steps. Ions found by L^{C}/MS [M+H]+=468.2.

### Step c

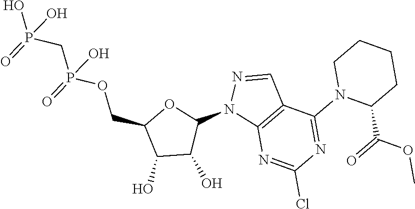

The acetonide from the previous step (175 mg, 0.37 mmol) in THE (5 mL) was added, dropwise, to a stirring mixture of methylene (bis phosphonic dichloride) (467 mg, 1.87 mmol) and DIEA (53 mg, 0.41 mmol) in THE (5 mL) were cooled to 0° C. via an ice bath. When the addition was complete the ice bath was removed and the reaction was stirred for 3 hours at ambient temperature. The mixture was then cooled to 0° C. via an ice bath and 0.5 N aqueous HCl (10 mL) was added. The mixture was stirred at 0° C. for 15 minutes then at ambient temperature for 3 hours (monitored by L^{C}/MS). The solvent was reduced to ˜0 5 mL on the rotary evaporator and the crude mixture was purified by reversed phase HPLC (5-85% acetonitrile in DI water, 0.1% TFA modifier, 25 minute gradient). The pure fractions were pooled and lyophilized to afford the product as a white, hygroscopic solid. Yield 57%, 125 mg. L^{C}/MS [M+H]+=586.2

### Step d

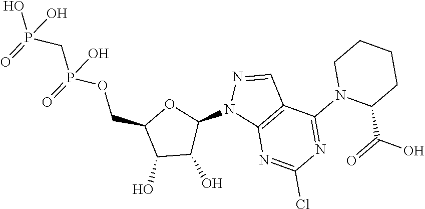 [SMILES: O=C(O)[C@H]1CCCCN1c1nc(Cl)nc2c1cnn2[C@@H]1O[C@H](COP(=O)(O)CP(=O)(O)O)[C@@H](O)[C@H]1O]

The intermediate from the previous step of this example (125 mg, 0.21 mmol) was stirred in a ½ mixture of methanol/DI water containing lithium hydroxide (30 mg, 1.3 mmol) at ambient temperature for 4 hours. The mixture was acidified with glacial acetic acid (1 mL), concentrated on the rotary evaporator and purified by reversed phase HPLC (5 to 80% acetonitrile in DI water, 0.1% TTFA modifier, 25 minute gradient). The pure fractions were pooled and lyophilized to afford the carboxylic acid as a hygroscopic white solid. Yield 84%, 106 mg. Ions found by L^{C}/MS [M+H]+=572.2.

### Step e

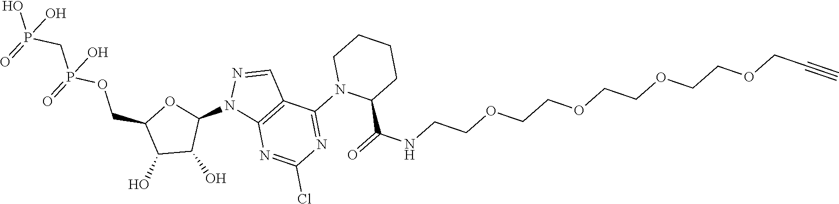 [SMILES: C#CCOCCOCCOCCOCCNC(=O)[C@@H]1CCCCN1c1nc(Cl)nc2c1cnn2[C@@H]1O[C@H](COP(=O)(O)CP(=O)(O)O)[C@@H](O)[C@H]1O]

EDC (50 mg, 0.26 mmol) was added to a mixture of the carboxylic acid intermediate from the previous step (106 mg, 0.19 mmol), propargyl-peg4 amine (60 mg, 0.26 mmol), and triethylamine (44 mg, 0.43 mmol) in DMF (2 mL). The reaction was stirred at ambient temperature for 4 hours and applied directly to reversed phase HPLC (5 to 85% acetonitrile in DI water, 0.1% TTFA modifier, 25 minute gradient). The pure fractions were pooled and lyophilized to afford the carboxylic acid as a hygroscopic white solid. Yield 54%, 93 mg. Ions found by L^{C}/MS [M+H]+=786.2.

### Step f

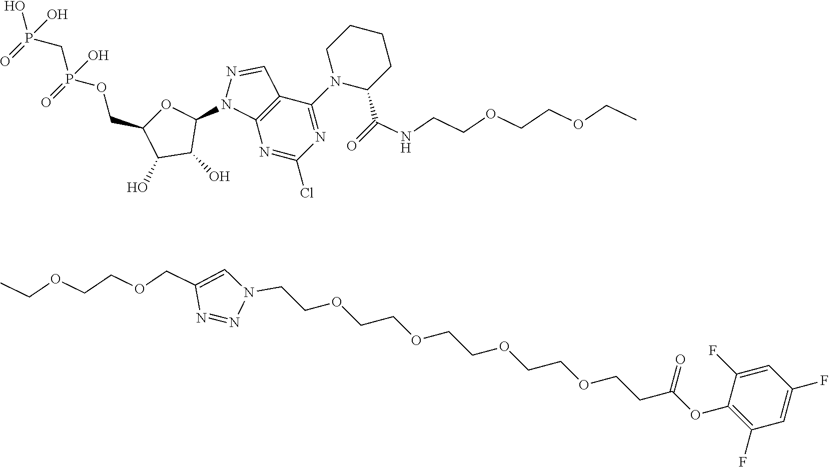

The product from the previous step (250 mg, 0.31 mmol) and intermediate A (161 mg, 0.38 mmol), were dissolved in DMF (1 mL) and cooled to 0° C. via an ice water bath. Copper sulfate (8 mg, 0.048 mmol) was added to a mixture of BTTA (34 mg, 0.08 mmol) and sodium ascorbate (189 mg, 0.96 mmol) in DI water (2 mL) the mixture was gently shaken for 15-20 seconds until the solution was clear. The copper mixture was added the alkyne/azido mixture and the reaction was stirred at 0 C for 10 minutes then at ambient temperature for 20 minutes. The crude reaction mixture was applied directly to reversed phase HPLC (5-85% acetonitrile in DI water, 0.1% TFA modifier, 25 minute gradient). The pure fractions were pooled and lyophilized to afford the product as a white, hygroscopic solid. Yield 215 mg, 56%. Ion(s) found by L^{C}/MS [(M/2)+H]+=603.8.

### Synthesis of Conjugate 11b

The title compound was prepared analogously to Conjugate 70 where starting material used in Int-110 was replaced by Int-16. Maldi TOF analysis of the purified final product gave an average mass of 62,471 Da (DAR=4.0).

### Synthesis of Conjugate 11a

The title compound was prepared analogously to Conjugate 4a where the starting material used in Int-110 was replaced by Int-16. Maldi TOF analysis of the purified final product gave an average mass of 62,404 Da (DAR=4.2).

### Synthesis of Int-27

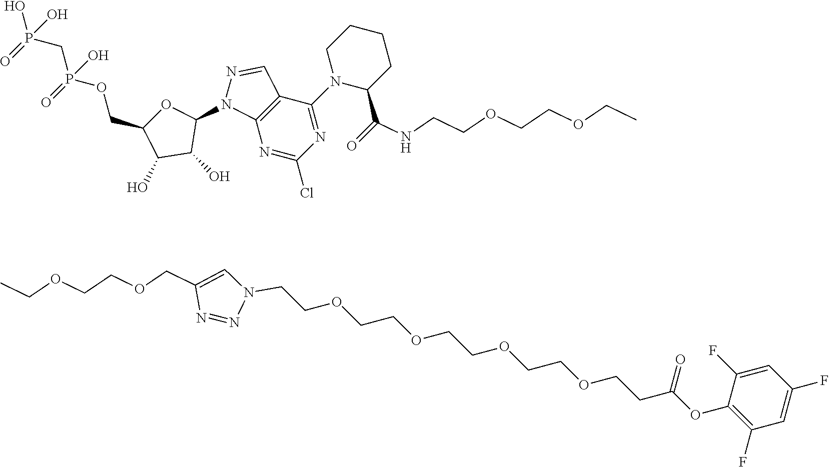

The title compound was prepared as described for Int-15. Ion(s) found by L^{C}/MS [M+H]+=603.8.

### Synthesis of Conjugate 16

The title compound was prepared analogously to Conjugate 70 where the starting material used in Int-110 was replaced by Int-27. Maldi TOF analysis of the purified final product gave an average mass of 60,745 (DAR=2.4).

### Synthesis of Int-106

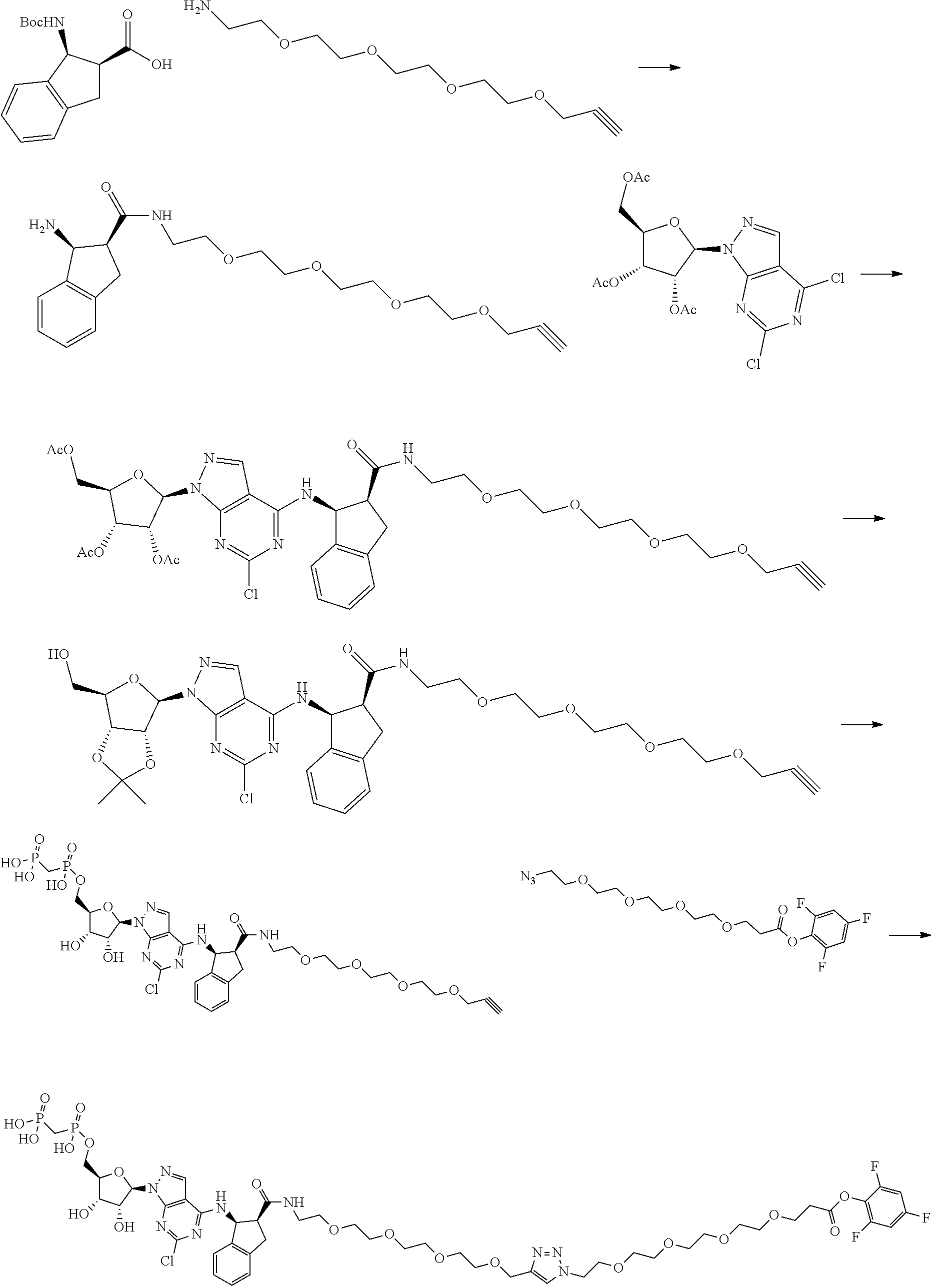

### Step a

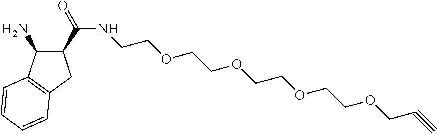 [SMILES: C#CCOCCOCCOCCOCCNC(=O)[C@H]1Cc2ccccc2[C@H]1N]

HATU (411 mg, 1.08 mmol) was added to a mixture of (racemic)-cis 1-tert-butoxycarbonylaminoindan-2-carboxylic acid (250 mg, 0.90 mmol), propargyl-peg4 amine (312 mg, 1.35 mmol), and DIEA (349 mg, 2.70 mmol) in DMF (2 mL). The reaction was stirred at ambient temperature for 4 hours and applied directly to reversed phase HPLC (5 to 85% acetonitrile in DI water, 0.1% TTFA modifier, 25 minute gradient). The pure fractions were pooled and lyophilized. The boc protected amine was stirred in a 1/1 mixture of TFA/DCM (10 mL) at ambient temperature for 30 minutes. The solvent was removed on a rotary evaporator and dried under high vacuum to afford the intermediate, TEA salt (racemic) as a clear viscous oil. Yield 71%, 2 steps, 325 mg. Ions found by L^{C}/MS [M+H]+=391.2.

### Step b

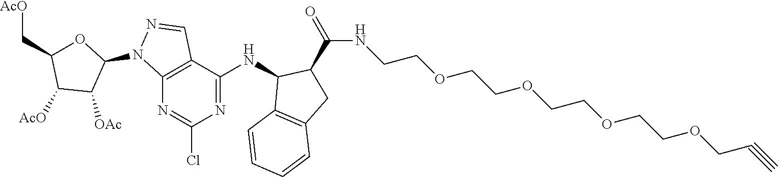 [SMILES: C#CCOCCOCCOCCOCCNC(=O)[C@H]1Cc2ccccc2[C@H]1Nc1nc(Cl)nc2c1cnn2[C@@H]1O[C@H](COC(C)=O)[C@@H](OC(C)=O)[C@H]1OC(C)=O]

Intermediate B (200 mg, 44 mmol), the intermediate described in the previous step of this example (175 mg, 0.45 mmol) and triethylamine (90 mg, 0.89 mmol) were stirred in ethanol (25 mL) at 50° C. for 4 hours. The mixture was cooled to ambient temperature and concentrated. The crude residue was purified by silica gel chromatography (0-10% methanol in DCM, 25 min) to afford the product as a pair of diastereomers. Yield 70%, 250 mg. Ions found by L^{C}/MS [M+H]+=801.2.

### Step c

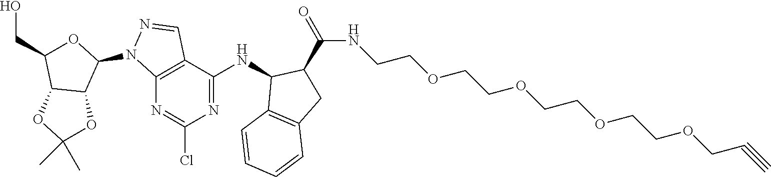 [SMILES: C#CCOCCOCCOCCOCCNC(=O)[C@H]1Cc2ccccc2[C@H]1Nc1nc(Cl)nc2c1cnn2[C@@H]1O[C@H](CO)[C@H]2OC(C)(C)O[C@H]21]

The triacetate product from the previous step (250 mg, 0.31 mmol) and potassium carbonate (30 mg) were stirred in methanol (30 mL) at ambient temperature for 2 hours. The mixture was filtered and neutralized with glacial acetic acid (0.5 mL) and concentrated. The crude residue was purified by silica gel chromatography (0-10% methanol in DCM, 25 min). The triol was taken up in DMF (2 mL) and 2,2 dimethoxy propane (5 mL), p-toluene sulfonic acid hydrate (15 mg) was added to the reaction and the mixture was stirred at 70° C. for 1 hour. Triethylamine (0.5 mL) was added to the reaction and the mixture was concentrated on the rotary evaporator. The crude residue was purified by silica gel chromatography (0-10% methanol in DCM, 25 min) to afford the product as a pair of diastereomers. Yield 60%, 2 steps 135 mg. Ions found by L^{C}/MS [M+H]+=715.2.

### Step d

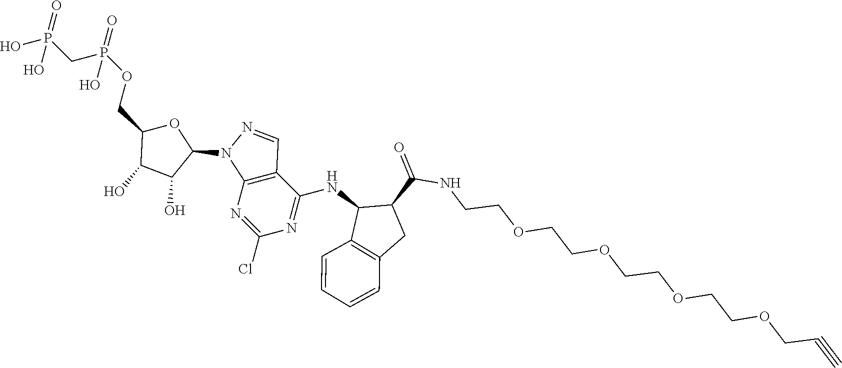 [SMILES: C#CCOCCOCCOCCOCCNC(=O)[C@H]1Cc2ccccc2[C@H]1Nc1nc(Cl)nc2c1cnn2[C@@H]1O[C@H](COP(=O)(O)CP(=O)(O)O)[C@@H](O)[C@H]1O]

The acetonide from the previous step (200 mg, 0.27 mmol) in THE (5 mL) was added, dropwise, to a stirring mixture of methylene (bis phosphonic dichloride) (197 mg, 1.12 mmol) and DIEA (36 mg, 0.28 mmol) in THE (5 mL) were cooled to 0° C. via an ice bath. When the addition was complete the ice bath was removed and the reaction was stirred for 3 hours at ambient temperature. The mixture was then cooled to 0° C. via an ice bath and 0.5 N aqueous HCl (10 mL) was added. The mixture was stirred at 0° C. for 15 minutes then at ambient temperature for 3 hours (monitored by L^{C}/MS). The solvent was reduced to ˜5 mL on the rotary evaporator and the crude mixture was purified by reversed phase HPLC (5-85% acetonitrile in DI water, 0.1% TFA modifier, 25 minute gradient). The pure fractions were pooled and lyophilized to afford the product as a white, hygroscopic solid (pair of diastereomers). Yield 45%, 107 mg. Ions found by L^{C}/MS [M+H]+=833.2.

### Step e

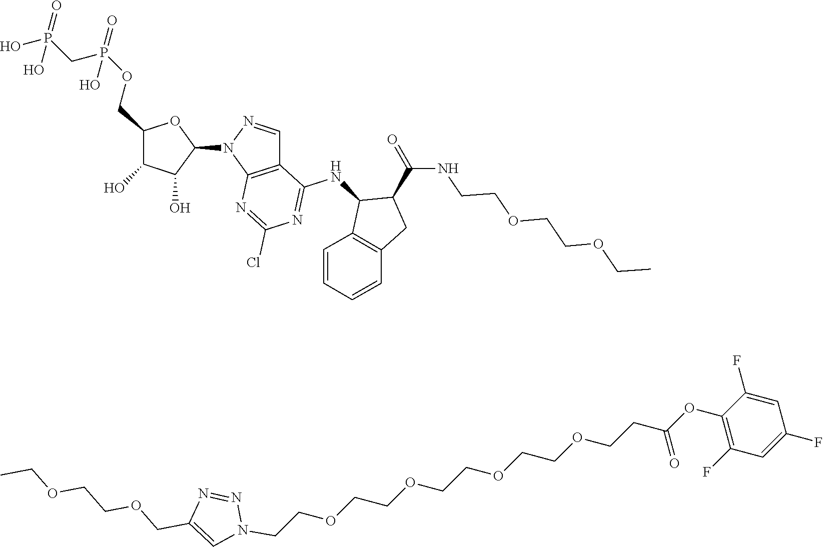

The product from the previous step (90 mg, 0.11 mmol) and intermediate A (46 mg, 0.11 mmol), were dissolved in DMF (1 mL) and cooled to 0° C. via an ice water bath. Copper sulfate (3 mg, 0.016 mmol) was added to a mixture of BTTA (12 mg, 0.027 mmol) and sodium ascorbate (64 mg, 0.32 mmol) in DI water (2 mL) the mixture was gently shaken for 15-20 seconds until the solution was clear. The copper mixture was added to the alkyne/azido mixture and the reaction was stirred at 0° C. for 10 minutes then at ambient temperature for 20 minutes. The crude reaction mixture was applied directly to reversed phase HPLC (5-85% acetonitrile in DI water, 0.1% TFA modifier, 25 minute gradient). The pure fractions were pooled and lyophilized to afford the diastereomeric mixture as a white, hygroscopic solid. Yield 104 mg, 76%. Ions found by L^{C}/MS [(M/2)+H]+=627.8.

### Synthesis of Conjugate 56

The title compound was prepared analogously to Conjugate 70 where starting material used in Int-110 was replaced by Int-106. Maldi TOF analysis of the purified final product gave an average mass of 63,241 Da (DAR=4.5).

### Synthesis of Int-61

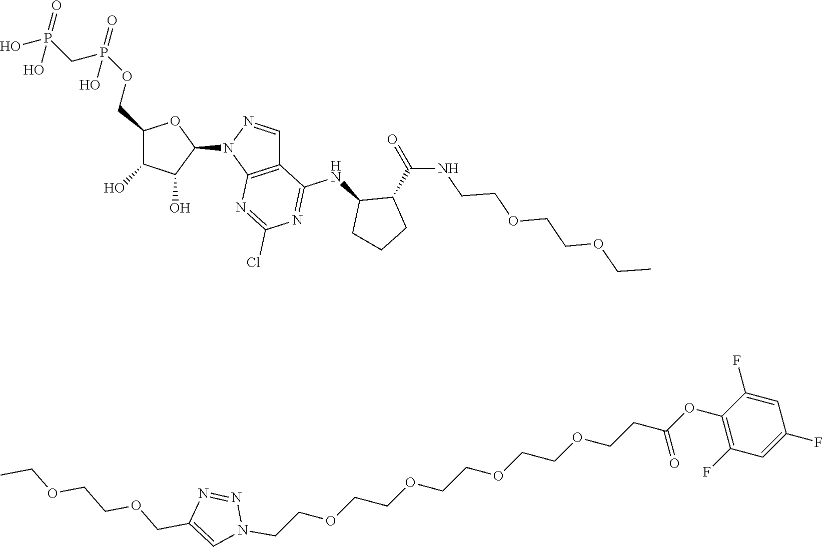

The title compound was prepared as described for Int-106 where (racemic)-cis 1-tert-butoxycarbonylamino-indan-2-carboxylic acid is replaced with (racemic) trans-boc-amino-cyclopentane carboxylic acid. Ion(s) found by L^{C}/MS [(M/2)+H]+=603.8.

### Synthesis of Conjugate 38

The title compound was prepared analogously to Conjugate 70 where starting material from Int-110 was replaced by Int-61. Maldi TOF analysis of the purified final product gave an average mass of 63,348 Da (DAR=4.8).

### Synthesis of Int-36

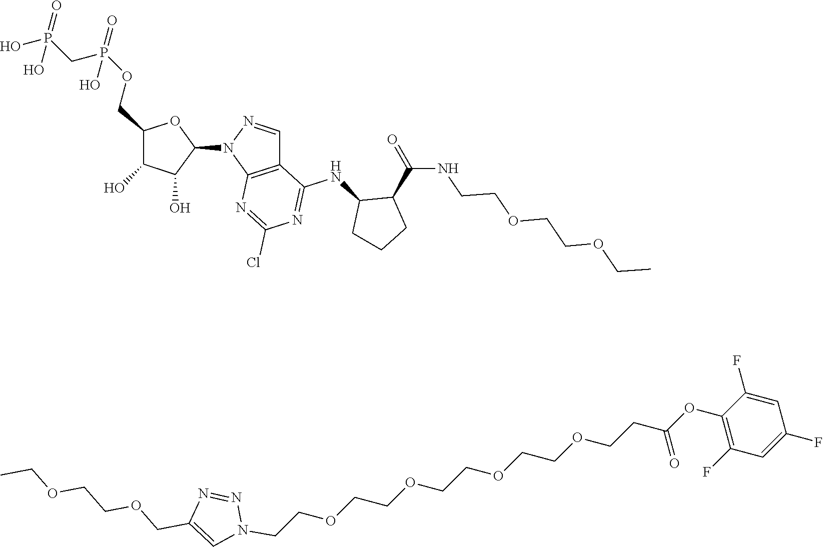

The title compound was prepared as described for Int-106 where (racemic)-cis 1-tert-butoxycarbonylamino-indan-2-carboxylic acid is replaced with (racemic) trans-Boc-amino-cyclopentane carboxylic acid. Ion(s) found by L^{C}/MS [(M/2)+H]+=603.8.

### Synthesis of Conjugate 19

The title compound was prepared analogously to Conjugate 70 where starting material used in Int-110 was replaced by Int-36. Maldi TOF analysis of the purified final product gave an average mass of 63,363 Da (DAR=4.8).

### Synthesis of Int-10

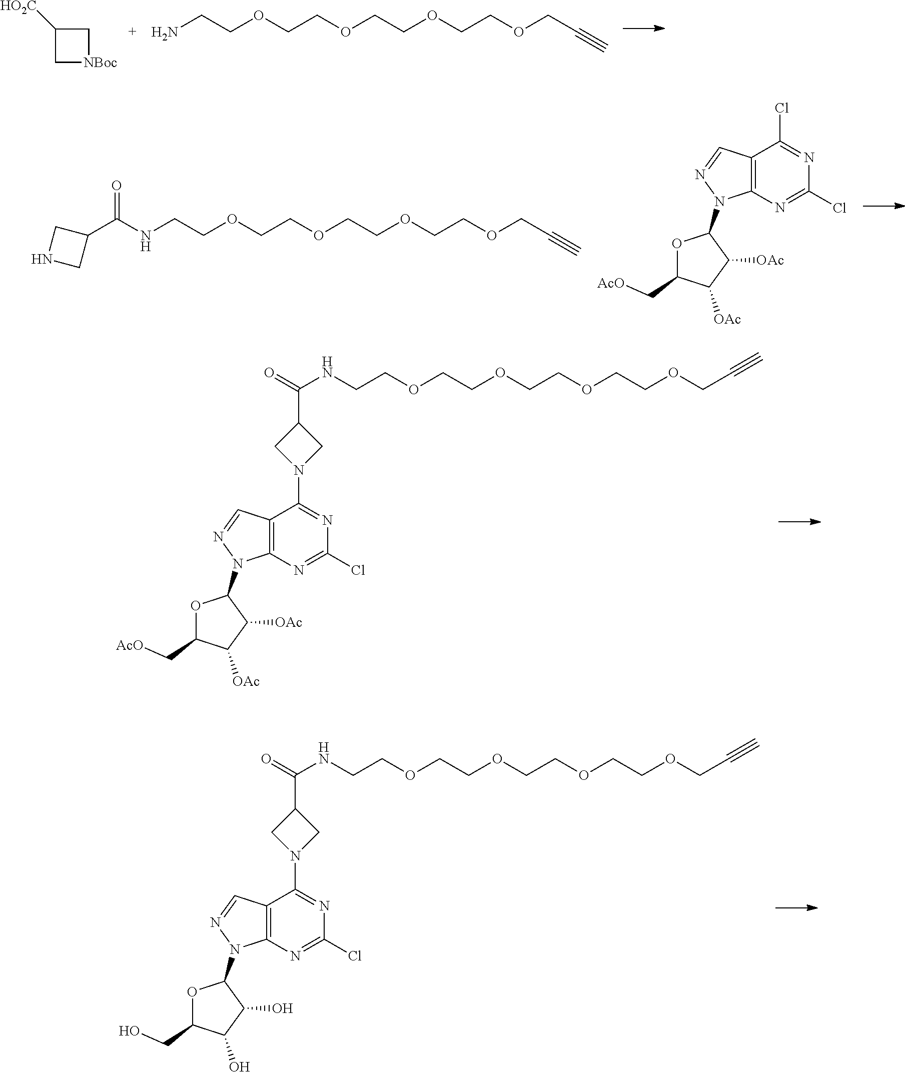 [SMILES: CC(C)(C)OC(=O)N1CC(C(=O)O)C1 ; C#CCOCCOCCOCCOCCN ; C#CCOCCOCCOCCOCCNC(=O)C1CNC1 ; CC(=O)OC[C@H]1O[C@@H](n2ncc3c(Cl)nc(Cl)nc32)[C@H](OC(C)=O)[C@@H]1OC(C)=O ; C#CCOCCOCCOCCOCCNC(=O)C1CN(c2nc(Cl)nc3c2cnn3[C@@H]2O[C@H](COC(C)=O)[C@@H](OC(C)=O)[C@H]2OC(C)=O)C1 ; C#CCOCCOCCOCCOCCNC(=O)C1CN(c2nc(Cl)nc3c2cnn3[C@@H]2O[C@H](CO)[C@@H](O)[C@H]2O)C1]

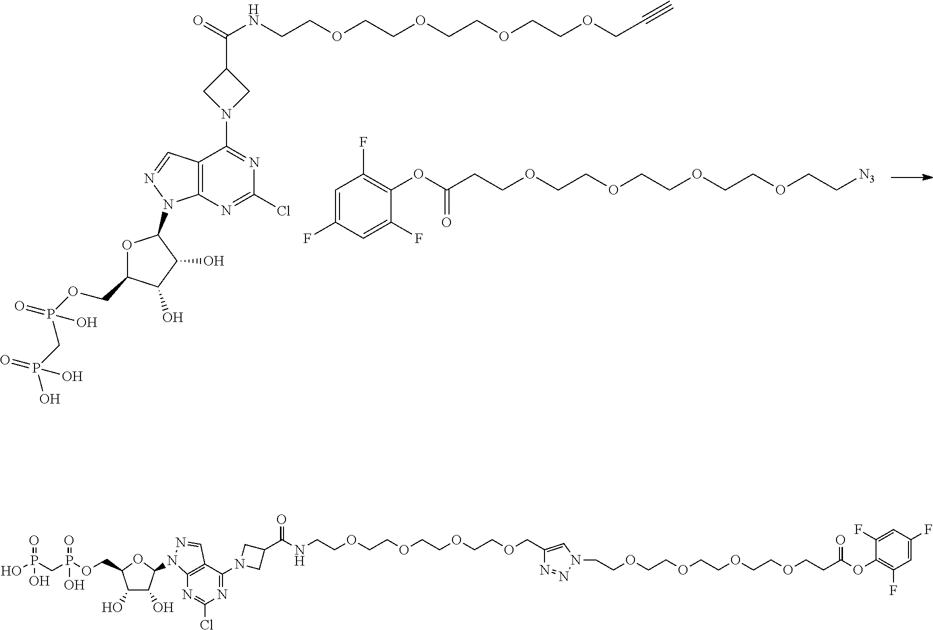 [SMILES: C#CCOCCOCCOCCOCCNC(=O)C1CN(c2nc(Cl)nc3c2cnn3[C@@H]2O[C@H](COP(=O)(O)CP(=O)(O)O)[C@@H](O)[C@H]2O)C1 ; [N-]=[N+]=NCCOCCOCCOCCOCCC(=O)Oc1c(F)cc(F)cc1F ; O=C(CCOCCOCCOCCOCCn1cc(COCCOCCOCCOCCNC(=O)C2CN(c3nc(Cl)nc4c3cnn4[C@@H]3O[C@H](COP(=O)(O)CP(=O)(O)O)[C@@H](O)[C@H]3O)C2)nn1)Oc1c(F)cc(F)cc1F]

### Step a

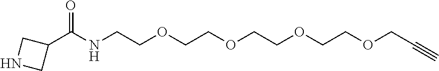 [SMILES: C#CCOCCOCCOCCOCCNC(=O)C1CNC1]

1-[(Tert-butyl)oxycarbonyl]azetidine-3-carboxylic acid (500 mg, 2.48 mmol), HATU (1.42 g, 3.72 mmol) and DIEA (0.88 mL, 2.48 mmol) in DMF (6 mL) (0.88 mL, 2.48 mmol) were stirred together for 10 minutes at ambient temperature. To this propargyl-peg4-amine (862 mg, 3.72 mmol) in DMF (1 mL) was added and the resulting mixture was stirred for 1 hour. The reaction was concentrated and purified by reversed phase HPLC (5% to 100% ACN/water) yielded the boc-protected intermediate as a yellow viscous liquid. The boc-protected intermediate was taken up in DCM (10 mL) and treated with 4M aqueous HCl in dioxane (8 mL) for 3 hours. Removal of the solvent under reduced pressure followed drying under high vacuum afforded the amine-HCl salt as a yellowish viscous oil. Yield 627 mg, 99%. Ions found by LCMS: [M+H]⁺=315.9.

### Step b

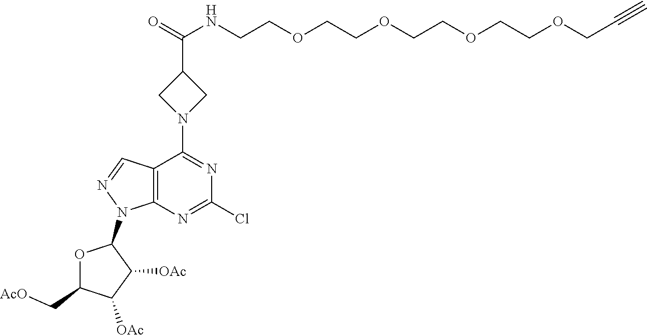 [SMILES: C#CCOCCOCCOCCOCCNC(=O)C1CN(c2nc(Cl)nc3c2cnn3[C@@H]2O[C@H](COC(C)=O)[C@@H](OC(C)=O)[C@H]2OC(C)=O)C1]

A mixture of intermediate B (447 mg, 1.118 mmol), the product from the previous step (456 mg, 1.34 mmol), and triethylamine (0.31 mL) in methanol (6 mL) were heated at 50° C. for 2 hours. After complete consumption of the starting materials, the reaction mixture was cooled to room temperature and volatiles were removed by rotatory evaporation. The crude residue was redissolved in ethyl acetate (50 mL) and washed with water and brine. The combined organic layer was dried over sodium sulfate, filtered and solvent was removed under reduced pressure to yield the compound which was used for the next step without further purification. Ions found by LCMS: [M+H]⁺=724.6.

### Step c

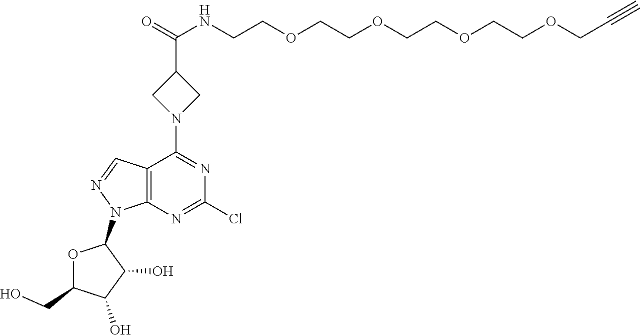

The triacetate from the previous step (800 mg, 1.118 mmol) was re-dissolved in methanol (6 mL) and potassium carbonate (540 mg, 3.91 mmol) was added, and the reaction stirred at ambient temperature for 2 hours. The reaction mixture was filtered through celite, and the filter cake was washed with methanol (2×20 mL). The solution was concentrated in vacuo to remove volatiles and the crude material thus obtained was purified by reversed phase HPLC (5% to 100% ACN/water, 0.1% TFA modifier) to yield the compound. white foam. Yield 300 mg, 45%. Ions found by LCMS: [M+H]⁺=598.8.

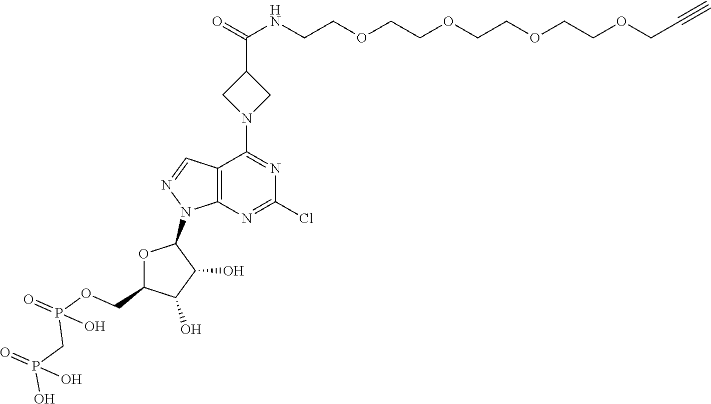

To a solution of the triol from the previous step (300 mg, 0.5 mmol) and 2,2-dimethoxypropane (0.31 mL, 2.5 mmol) in acetone (20 mL) at room temperature was added p-TsOH (8 mg, 0.05 mmol). The reaction was stirred for two hours then concentrated under reduced pressure. The crude oil was re-dissolved in ethyl acetate (10 mL) and washed with saturated sodium bicarbonate. The organic layer was separated and dried over sodium sulfate, filtered and concentrated in vacuo to provide acetonide derivative. white solid, which was used for the next step without further purifications. Ions found by LCMS: [M+H]⁺=639.8. To a suspension of methylenebis(phosphonic dichloride) (375 mg, 1.5 mmol) in THE (5 mL) at 0° C. was added DIEA (0.09 mL, 0.55 mmol). To the resulting mixture was added a solution of the acetonide in THE (2 mL) dropwise over the course of 10 minutes. Following addition, the resulting mixture was stirred at ambient temperature for an additional 1 hour, then the solution was transferred to a pre-cooled (0° C.) flask containing 0.2 M aqueous HCl (8 mL). The reaction mixture was warmed to ambient temperature and stirred for 2 hours. Upon completion, the reaction mixture was concentrated under reduced pressure and the crude material was purified by reverse phase HPLC using ACN: water (0.1% TFA modifier). The product was a white solid. Yield 243 mg, 81%. Ions found by LCMS: [M+H]⁺=757.8.

### Step e

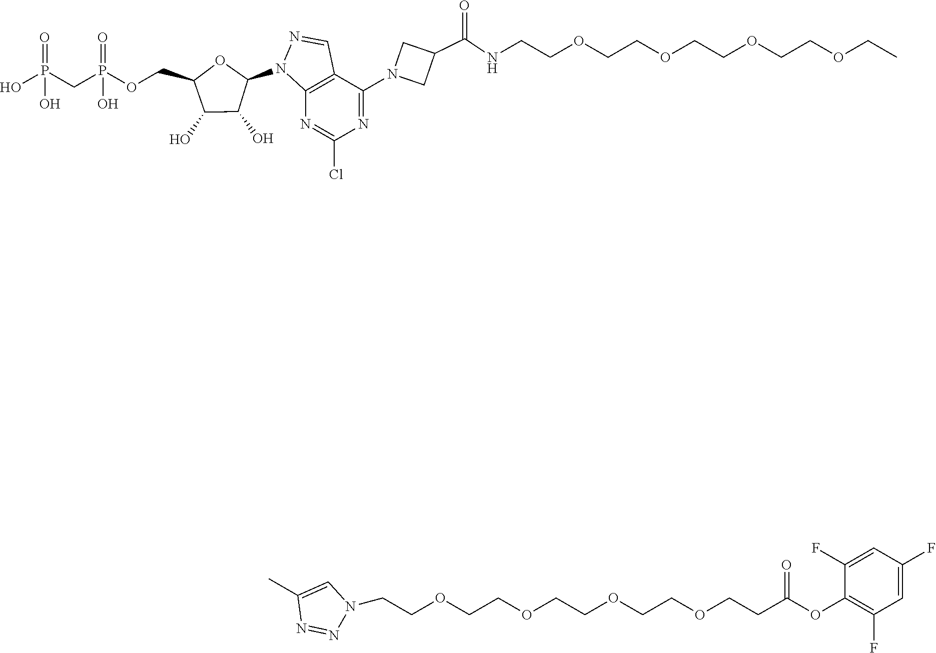

To a solution of the product from the previous step (50 mg, 0.066 mmol) and intermediate A (27 mg, 0.066 mmol) dissolved in DMF:H₂O (1:3, 1.5 mL) were cooled to 0° C. To this a pre-mixed solution of a solution of copper(II) sulfate (1 mg g, 0.007 mmol), sodium ascorbate (57 mg, 0.198 mmol), and BTTA (6 mg, 0.013 mmol) dissolved in water (0.5 mL) was added and stirred for 5 minutes at the same temperature and gradually warmed to room temperature and stirred room temperature for 15 minutes. When the reaction was complete (by HPLC), the mixture was quenched by the addition of few drops of glacial acetic acid and 125 mM EDTA (pH 6) and the product was purified by reversed phase HPLC (10% to 100% ACN/water, 0.1% TFA). White solid. Yield 142 mg, 93.8%. Ions found by LCMS [M+H]⁺=1179.4.

### Synthesis of Conjugate 8a

Trifluorophenol ester (13 mg, 0.011 mmol, described in Synthesis of Int-10) in DMF (0.5 mL) was added to Fc carrier SEQ ID NO: 17 (80 mg in 4 mL in PBS at pH 7.4) then adjusted to pH-8 with borate buffer (0.200 mL, pH 8.5, 1.0 M). The mixture was agitated at ambient temperature for 2 hours and then submitted for purification according to the general procedure. Maldi TOF analysis of the purified final product gave an average mass of 64,894 (DAR of 6.7). The conjugate was purified by buffer dialysis (PBS pH7.4) and SEC chromatography. Yield 54 mg, 77.1%.

### Synthesis of Conjugate 8b

Trifluorophenol ester (17 mg, 0.014481 mmol) described in Int-10 was conjugated to Fc carrier SEQ ID NO: 13 (100 mg, 0.00181 mmol, 5.13 mL in PBS at pH 7.4) as described in the synthesis of Conjugate 8a. Maldi TOF analysis of the purified final product gave an average mass of 65,370 Da (DAR=6.9). Yield 80 mg, 80.6%.

### Synthesis of Int-9

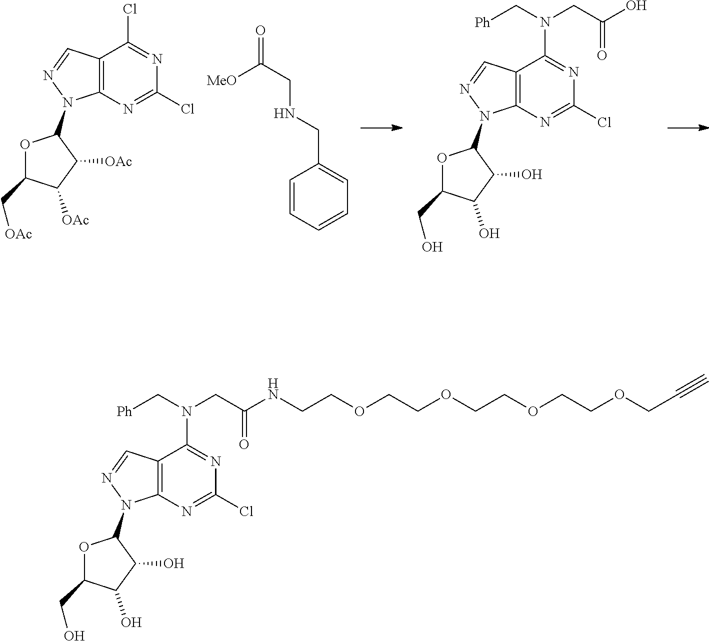

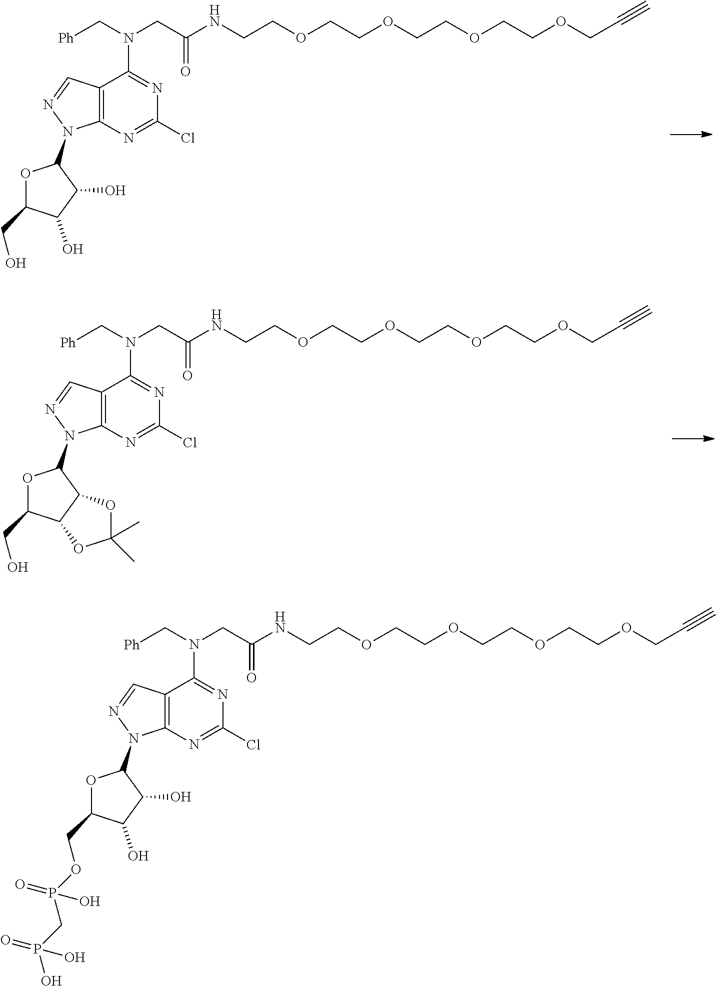

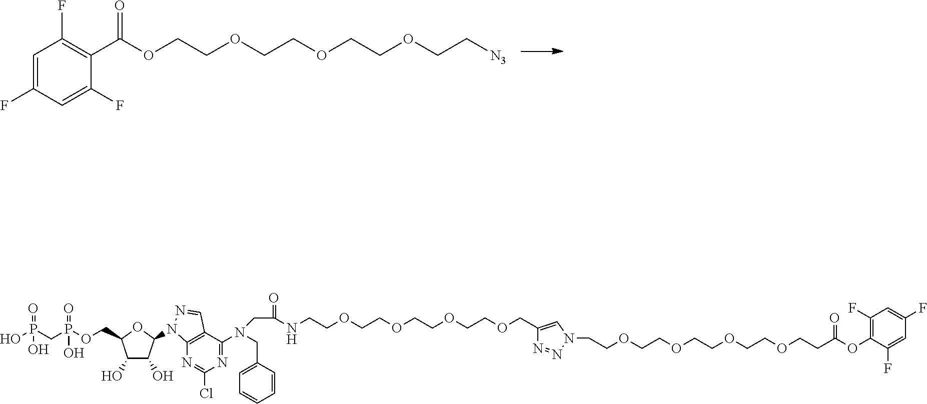 [SMILES: [N-]=[N+]=NCCOCCOCCOCCOC(=O)c1c(F)cc(F)cc1F ; O=C(CN(Cc1ccccc1)c1nc(Cl)nc2c1cnn2[C@@H]1O[C@H](COP(=O)(O)CP(=O)(O)O)[C@@H](O)[C@H]1O)NCCOCCOCCOCCOCc1cn(CCOCCOCCOCCOCCC(=O)Oc2c(F)cc(F)cc2F)nn1]

### Step a

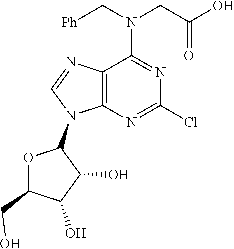

A mixture of intermediate B (447 mg, 1.112 mmol), methyl 2-[benzylamino]acetate (240 mg, 1.34 mmol), triethylamine (0.23 mL) and ethanol (6 mL) was heated at 50° C. for 1 hour. The mixture was cooled to ambient temperature and concentrated. The crude residue was redissolved in ethyl acetate (50 mL) and washed with water and brine. The organic layer was dried over sodium sulfate and solvent was removed under reduced pressure to yield the compound as a white solid (646 mg, 98%) which was used for the next step without further purification. Ions found by LCMS: [M+H]⁺=589.8. Crude ester (646 mg, 0.98 mmol) was dissolved in THE (5 mL) and to this aqueous 1M LiOH solution (11 mL) was added and stirred at room temperature until TLC showed completion of the reaction. The reaction mixture was concentrated and diluted with water and acidified with 1M HCl and extracted with ethyl acetate (3×20 mL). The combined organic extracts were washed with water, brine and dried over sodium sulfate. Removal of the solvent yielded crude product which was purified by reversed phase HPLC (5% to 100% ACN/water) to yield the desired compound as a white foam. Yield 392 mg, 88.8%. Ions found by LCMS: [M+H]⁺=449.8.

### Step b

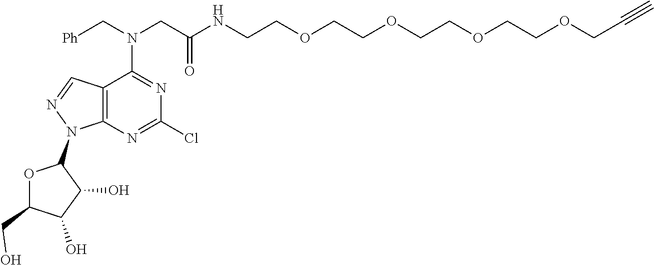

The carboxylic acid from the previous step (392 mg, 0.87 mmol), HATU (497 mg 1.31 mmol) and DIEA (0.23 mL, 1.31 mmol) in DMF (5 mL) were stirred together for 10 minutes at ambient temperature. Propargyl-PEG₄-amine (241 mg, 1.04 mmol) in DMF (1 mL) were added and the resulting mixture was stirred at room temperature for 1 hour then concentrated under reduced pressure. The crude material was purified by reverse phase HPLC (5% ACN: water, 100% ACN) to yield the desired compound. Yield of white solid 466 mg, 81%. Ions found by LCMS: [M+H]⁺=662.8.

### Step c

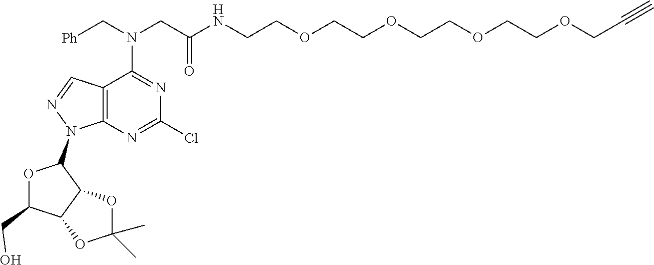

To the tri-hydroxy product from the previous step (497 mg, 0.75 mmol) and 2,2-dimethoxypropane (0.46 mL, 3.75 mmol) in acetone (20 mL) at room temperature was added ε-TsOH (13 mg, 0.075 mmol). The reaction was stirred for two hours at ambient temperature then concentrated under reduced pressure. The crude product was dissolved in ethyl acetate (10 mL) and washed with saturated sodium bicarbonate. The organic layer was separated and dried over sodium sulfate, filtered and concentrated in vacuo to provide an off-white solid which was used for the next step without further purifications. Yield 497 mg, 100%. Ions found by LCMS: [M+H]⁺=702.8.

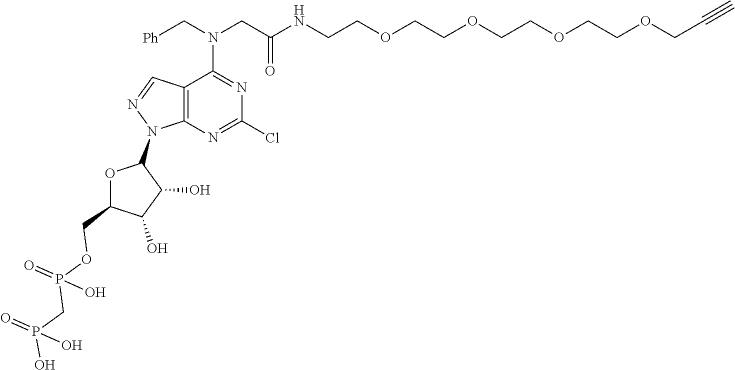

To a suspension of methylenebis(phosphonic dichloride) (562 mg, 2.25 mmol) in THE (5 mL) at 0° C. was added DIPEA (0.14 mL, 0.83 mmol). To the resulting mixture was added a solution of the acetonide from the previous step (497 mg, 0.75 mmol) in THE (2 mL) dropwise over the course of 1 hour. Following addition, the resulting mixture was stirred at 0° C. for an additional 15 minutes, then the solution was transferred to a pre-cooled (0° C.) flask containing 0.2 M aqueous HCl (2.25 mL). The reaction mixture was warmed to ambient temperature and stirred for 4 hours. Upon completion (monitored by LCMS), the reaction mixture was concentrated and purified by reverse phase HPLC using 5% ACN/water to 100% ACN/water with 0.1% TFA modifier. White solid Yield 454 mg, 74%. Ions found by LCMS: [M+H]⁺=820.6.

### Step e

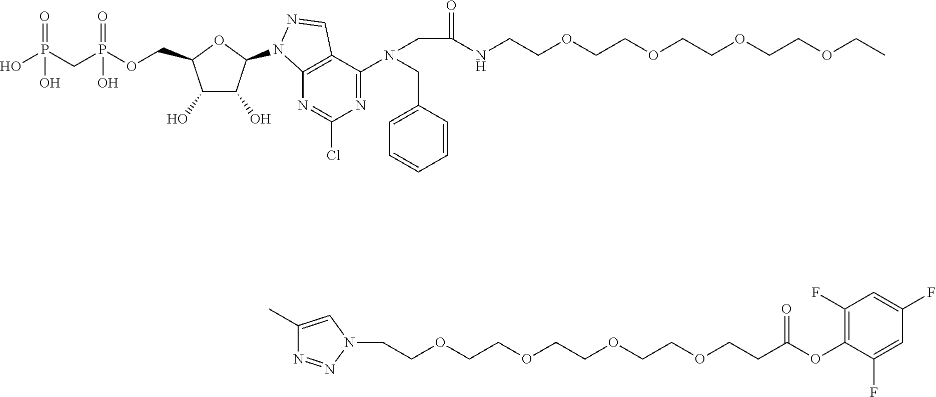

To a solution of the product from step c (0.1 g, 0.122 mmol), and intermediate A (0.037 g, 0.088 mmol) dissolved in DMF:H₂O (1:3, 1.5 mL) was cooled to 0° C. To this a pre-mixed solution of a solution of copper(II) sulfate (0.0020 g, 0.0121 mmol), sodium ascorbate (0.072 g, 0.365 mmol), and BTTA (0.010 g, 0.0244 mmol) dissolved in water (0.5 mL) was added and stirred for 5 min at the same temperature and gradually warmed to room temperature and stirred room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). White solid (0.142 g, 93.84%). LCMS [(M+2H)/2]⁺=621.2.

### Synthesis of Conjugate 7a

Trifluorophenol ester (described in the synthesis of Int-9) was conjugated to Fc carrier SEQ ID NO: 17 as described in the general conjugation procedure. Maldi TOF analysis of the purified final product gave an average mass of 63,727 Da. (DAR=5.2). Yield 87.5 mg, 87.5%.

### Synthesis of Conjugate 7b

Trifluorophenol ester (9 mg, 0.0072 mmol) (described in the synthesis of Int-9) was conjugated to Fc carrier SEQ ID NO: 13 (50 mg, 2.56 mL in PBS at pH 7.4, 0.0009 1 mmol), as described in the synthesis of Conjugate 8a. Maldi TOF analysis of the purified final product gave an average mass of 63,528 Da (DAR=4.8). Yield 34.3 mg, 68.6%.

### Synthesis of Int-21

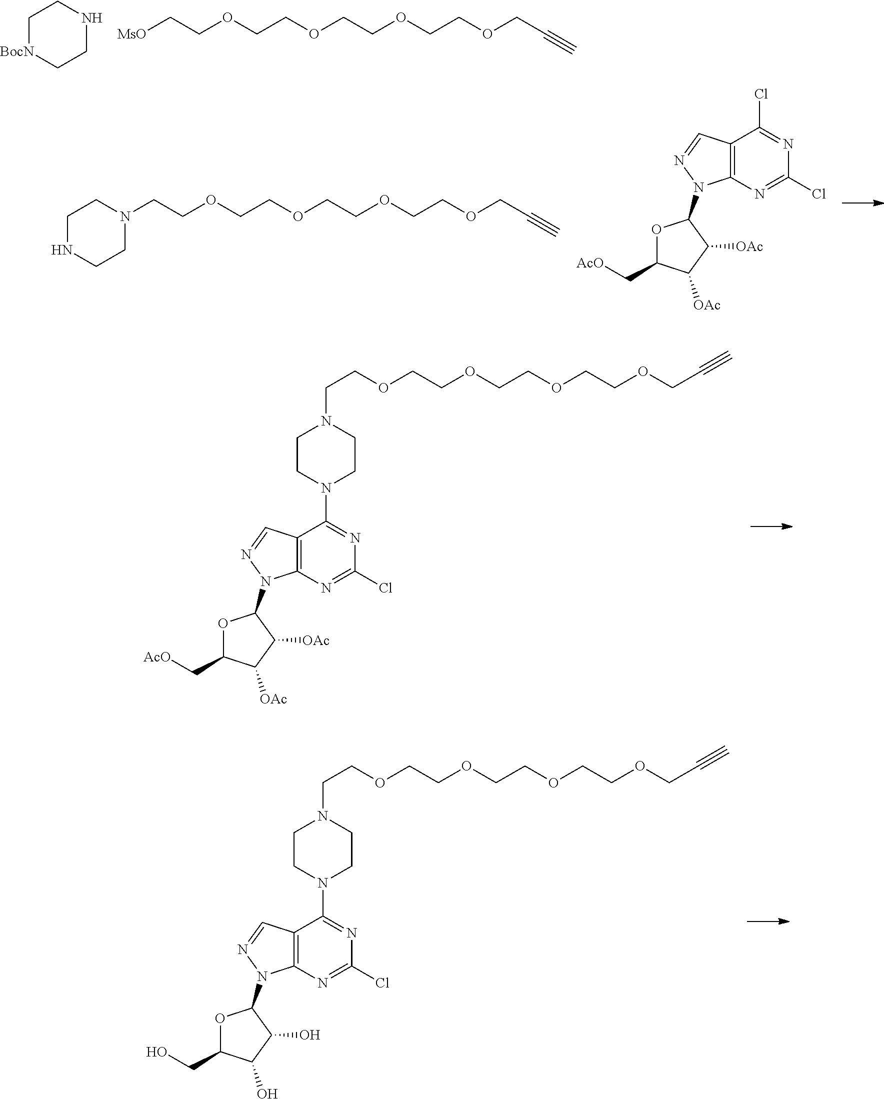 [SMILES: CC(C)(C)OC(=O)N1CCNCC1 ; C#CCOCCOCCOCCOCCOS(C)(=O)=O ; C#CCOCCOCCOCCOCCN1CCNCC1 ; CC(=O)OC[C@H]1O[C@@H](n2ncc3c(Cl)nc(Cl)nc32)[C@H](OC(C)=O)[C@@H]1OC(C)=O ; C#CCOCCOCCOCCOCCN1CCN(c2nc(Cl)nc3c2cnn3[C@@H]2O[C@H](COC(C)=O)[C@@H](OC(C)=O)[C@H]2OC(C)=O)CC1 ; C#CCOCCOCCOCCOCCN1CCN(c2nc(Cl)nc3c2cnn3[C@@H]2O[C@H](CO)[C@@H](O)[C@H]2O)CC1]

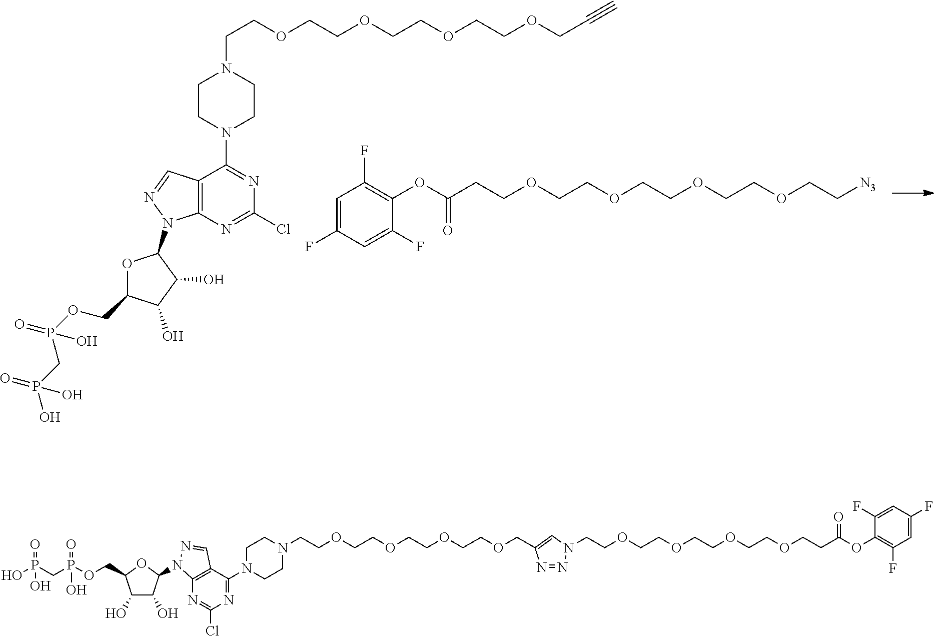 [SMILES: C#CCOCCOCCOCCOCCN1CCN(c2nc(Cl)nc3c2cnn3[C@@H]2O[C@H](COP(=O)(O)CP(=O)(O)O)[C@@H](O)[C@H]2O)CC1 ; [N-]=[N+]=NCCOCCOCCOCCOCCC(=O)Oc1c(F)cc(F)cc1F ; O=C(CCOCCOCCOCCOCCn1cc(COCCOCCOCCOCCN2CCN(c3nc(Cl)nc4c3cnn4[C@@H]3O[C@H](COP(=O)(O)CP(=O)(O)O)[C@@H](O)[C@H]3O)CC2)nn1)Oc1c(F)cc(F)cc1F]

### Step a

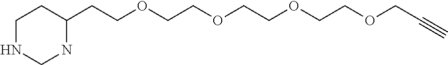 [SMILES: C#CCOCCOCCOCCOCCC1CCNCN1]

Propargyl-PEG-₄mesylate (931 mg, 3 mmol), N-Boc piperazine (558 mg, 3 mmol) and potassium carbonate (828 mg, 6 mmol) in acetonitrile (50 mL) were heated at reflux for 15 hours. The reaction mixture was cooled to ambient temperature and the solvent was removed under reduced pressure. The crude material was partitioned between water and ethyl acetate, the organic layer was separated, and the aqueous layer was extracted with ethyl acetate (2×10 mL), The combined organic extracts were washed with brine, water and dried over sodium sulfate Concentration of the solvent yielded the crude N-Boc protected product as a yellow viscous liquid. Ions found by LCMS: [M+H]⁺=401.9. N-Boc protected amine was re-dissolved in DCM and cooled in an ice-bath to 0° C. To this HCl in dioxane (4 mL, 10 equiv.) was added and gradually warmed the reaction mixture to ambient temperature and stirred until LCMS analysis indicated complete conversion of the starting material to product. The solvent was removed under reduced pressure and the crude material was dried under high vacuum to yield product as a white solid (HCl salt). Yield: 672 mg, 85%, 2 steps. Ions found by LCMS: [M+H]⁺=301.2.

### Step b

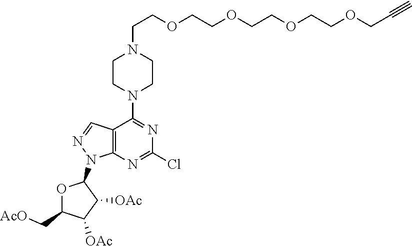

A mixture of intermediate B (250 mg, 0.56 mmol), amine from the previous step (201 mg, 0.67 mmol), and triethylamine (0.12 mL) in EtOH (6 mL) were heated at 50° C. for 2 hours. After complete consumption of the starting materials (by LCMS), the reaction mixture was cooled to ambient temperature and volatiles were removed on the rotatory evaporator to yield the crude material which was purified by normal phase column chromatography using hexanes: ethyl acetate. The product was a white solid. Yield 200 mg, 50%. Ions found by LCMS: [M+H]⁺=710.8.

### Step c

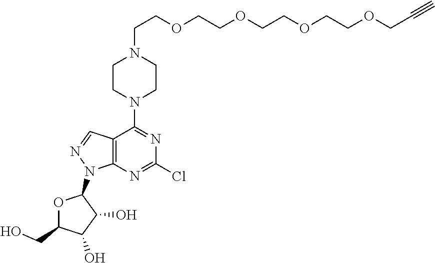

The triacetate product from the previous step (220 mg, 0.31 mmol) was dissolved in methanol (10 mL) and treated with potassium carbonate (149 mg, 1.08 mmol) then stirred at ambient temperature for 1.5 hours. The reaction mixture was filtered through celite, and the filter cake was washed with methanol (3×20 mL). The solution was concentrated in vacuo to remove volatiles to give crude product, which was purified by reverse phase HPLC to yield the product. The product was a white solid. Yield 139 mg, 77%. Ions found by LCMS: [M+H]⁺=585.2.

### Step d

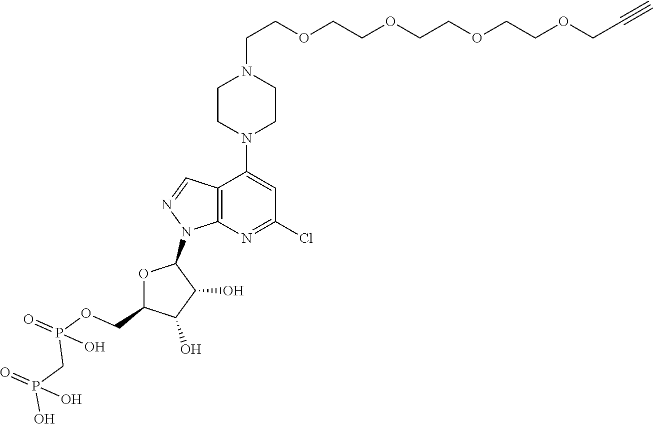

To a solution of the product from the previous step (139 mg, 0.24 mmol) and 2,2-dimethoxypropane (0.145 mL, 1.188 mmol) in acetone (10 mL) at ambient temperature was added p-TsOH (23 mg, 0.005 mmol). The reaction was stirred for two hours then concentrated under reduced pressure to afford the acetonide intermediate. The crude material was dissolved in ethyl acetate (20 mL) and washed with saturated sodium bicarbonate. The organic layer was separated and dried over sodium sulfate, filtered and concentrated in vacuo to provide the acetonide derivative as a white solid which was used for the next step without further purification. Ions found by LCMS: [M+H]⁺=624.8. To a suspension of methylenebis(phosphonic dichloride) (178 mg, 0.71 mmol) in THE (5 mL) at 0° C. was added DIEA (0.046 mL, 0.26135 mmol). To the resulting mixture was added a solution of the acetonide (148 mg, 0.24 mmol) in THE (2 mL) dropwise over the course of 10 minutes. Following the addition, the resulting mixture was stirred at 0° C. for an additional 15 minutes, then the solution was transferred to a pre-cooled (0° C.) flask containing 0.2 M aqueous HCl (1.5 mL). The reaction mixture was warmed to ambient temperature and stirred for 4 hours. Upon completion (by LCMS), the reaction mixture was concentrated purified by reversed phase HPLC. The product was a white solid. Yield 100 mg 57%. Ions found by LCMS: [M+H]⁺=743.6.

### Step e

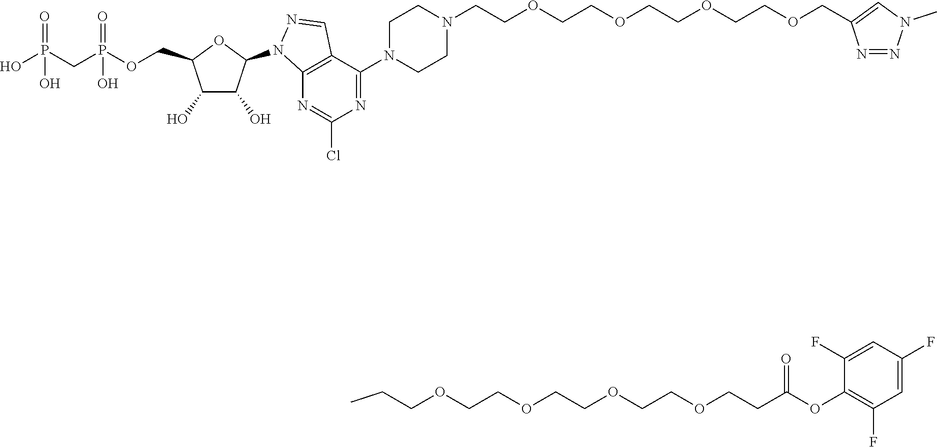

To a solution of the product from step d (0.07 g, 0.0942 mmol), and intermediate A (0.04 g, 0.0942 mmol) dissolved in DMF:H₂O (1:3, 1.5 mL) was cooled to ° C. To this a pre-mixed solution of a solution of copper(II) sulfate (0.0015 g, 0.00942 mmol), sodium ascorbate (0.056 g, 0.2826 mmol), and BTTA (0.019 g, 0.0188 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). White solid (0.07 g, 63.81%). LCMS[M+H]⁺=1165.4.

### Synthesis of Conjugate 13

Trifluorophenol ester (described in the synthesis of Int-21) (0.017 g, 0.01448 mmol) was conjugated to Fc carrier SEQ ID NO: 13 (0.1 g, 0.0018 mmol) as described in the synthesis of Conjugate 8a. Maldi TOF analysis of the purified final product gave an average mass of 62149 Da. (DAR=3.9). Yield 42.3 mg, 42.3%.

### Synthesis of Int-55

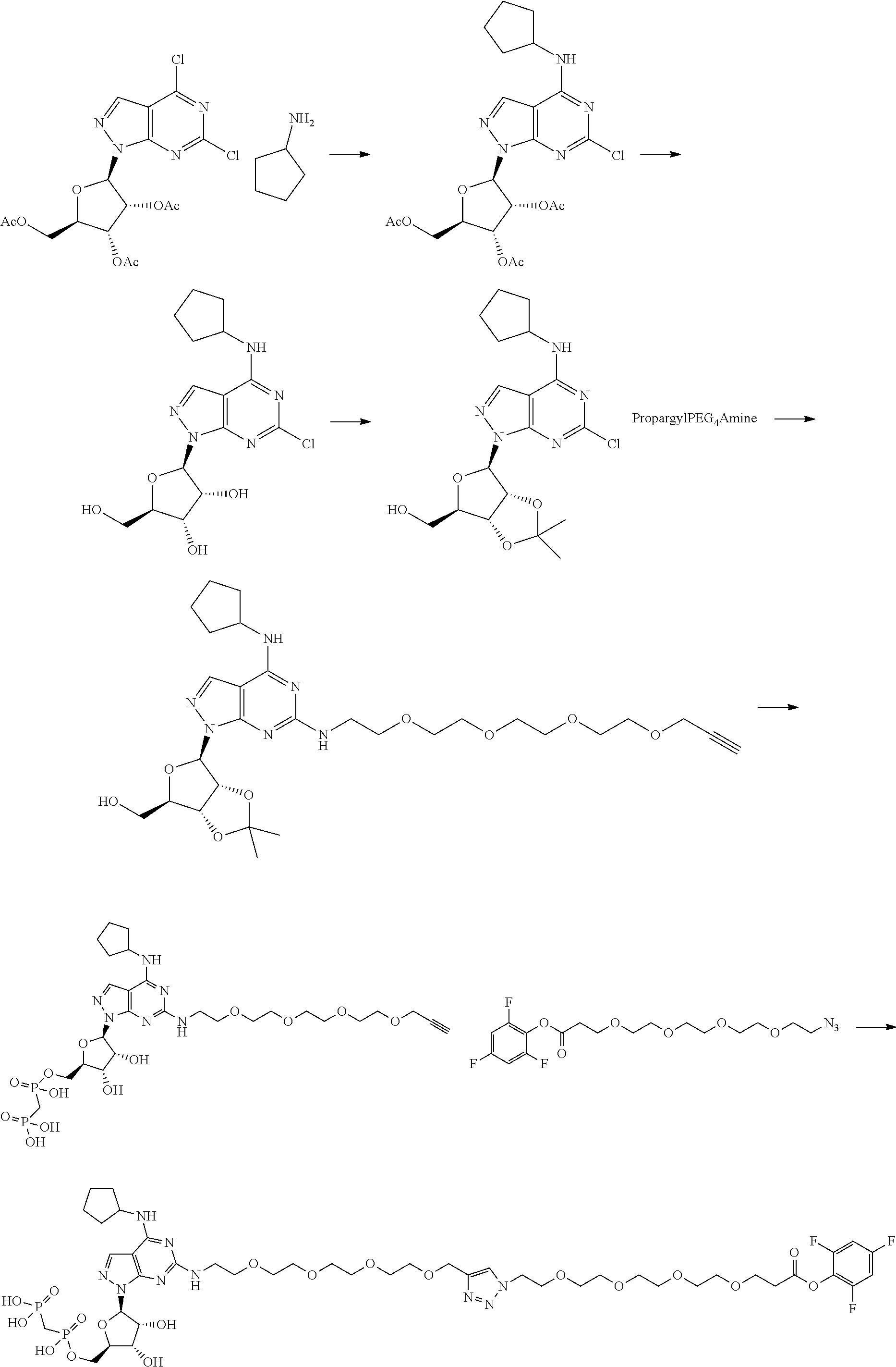

### Step a

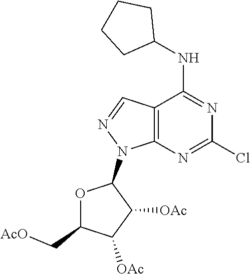 [SMILES: CC(=O)OC[C@H]1O[C@@H](n2ncc3c(NC4CCCC4)nc(Cl)nc32)[C@H](OC(C)=O)[C@@H]1OC(C)=O]

A mixture of intermediate B (1 g, 2.24 mmol), cyclopentylamine (228 mg, 2.68 mmol), triethylamine (0.46 mL) and ethanol (20 mL) were heated at 50° C. for 1 hour. The mixture was cooled to ambient temperature and concentrated. The residue was purified by column chromatography (petroleum ether/ethyl acetate: 5:1) to provide the product. The product was a white solid. Yield 1.1 g, 98%. Ions found by LCMS: [M+H]⁺=495.8.

### Step b

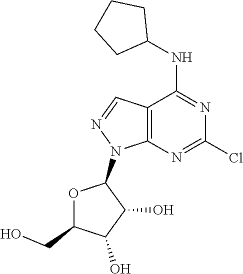 [SMILES: OC[C@H]1O[C@@H](n2ncc3c(NC4CCCC4)nc(Cl)nc32)[C@H](O)[C@@H]1O]

The triacetate product from the previous step (1.1 g, 2.21 mmol) was dissolved in THE (30 mL) and treated with a 2M aqueous LiOH solution (9 mL) at ambient temperature for 2 hours. The reaction mixture was concentrated under reduced pressure then acidified with 1 N aqueous HCl. The crude material was purified by reversed phase HPLC (10% to 100% ACN/water, 0.1% TFA). The product was a white solid. Yield 735 mg, 89%. Ions found by LCMS: [M+H]⁺=369.8.

### Step c

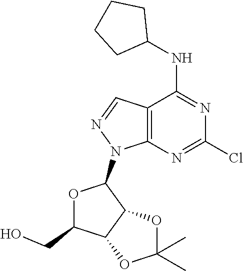 [SMILES: CC1(C)O[C@@H]2[C@H](O1)[C@@H](CO)O[C@H]2n1ncc2c(NC3CCCC3)nc(Cl)nc21]

To a solution of the product from the previous step (735 mg, 1.99 mmol) and 2,2-dimethoxypropane (1.22 mL, 9.94 mmol) in acetone (25 mL) at room temperature was added p-TsOH (34 mg, 0.198 mmol). The reaction was stirred for two hours then concentrated under reduced pressure. The crude product was dissolved in ethyl acetate (30 mL) and washed with saturated sodium bicarbonate. The organic layer was separated and dried over sodium sulfate, filtered, and concentrated under reduced pressure to yield an off-white solid, which was purified by silica gel chromatography using hexanes/ethyl acetate. The product was a white solid. Yield 690 mg, 85%. Ions found by LCMS: [M+H]⁺=410.9.

### Step d

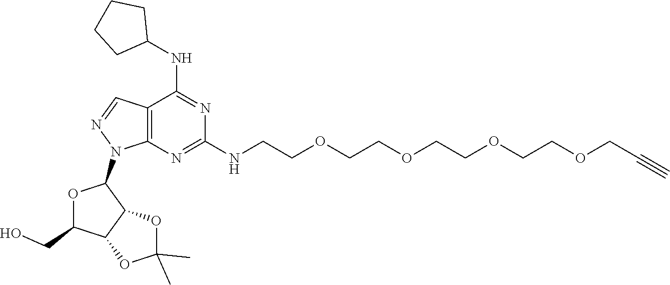 [SMILES: C#CCOCCOCCOCCOCCNc1nc(NC2CCCC2)c2cnn([C@@H]3O[C@H](CO)[C@H]4OC(C)(C)O[C@H]43)c2n1]

A mixture of the acetonide from the previous step (300 mg, 0.731 mmol), propargyl-peg₄ amine (203 mg, 0.88 mmol) and triethylamine (0.15 mL, 1.09 mmol) in ethanol (5 mL) were heated at 80° C. for 24 hours. The mixture was cooled to ambient temperature and concentrated. The residue was purified by reversed phase HPLC (10% to 100% ACN/water, 0.1% TFA) to provide the title compound. brownish viscous liquid. Yield 100 mg, 23%. Ions found by LCMS: [M+H]⁺=604.9.

### Step e

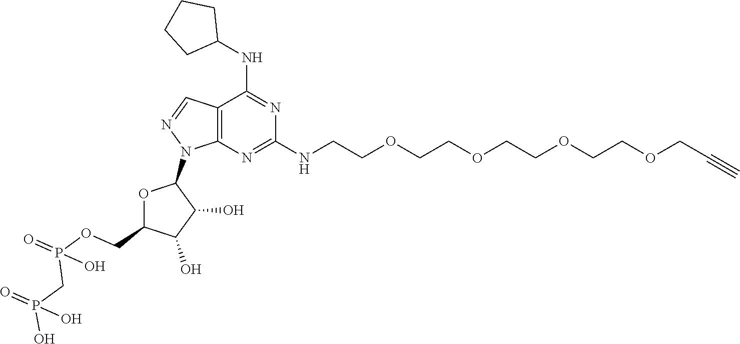

To a suspension of methylenebis(phosphonic dichloride) (124 mg, 0.496 mmol) in THE (5 mL) at 0° C. was added DIEA (0.032 mL, 0.18 mmol). To the resulting mixture was added to a solution of acetonide from the previous step (100 mg, 0.17 mmol) in THE (1 mL) dropwise over the course of 10 minutes. Following addition, the resulting mixture was stirred at 0° C. for an additional 15 minutes, and then the solution was added dropwise to a pre-cooled (0° C.) flask containing 0.2 M aqueous HCl (4.5 mL). The reaction mixture was warmed to ambient temperature and stirred for 2 hours. Upon completion (by LCMS), the reaction mixture was concentrated under reduced pressure and the crude material was purified by reverse phase HPLC (10% to 100% ACN/water, 0.1% TFA). The product was a white solid. Yield 72 mg, 60%. Ions found by LCMS: [M+H)]⁺=722.6.

### Step f

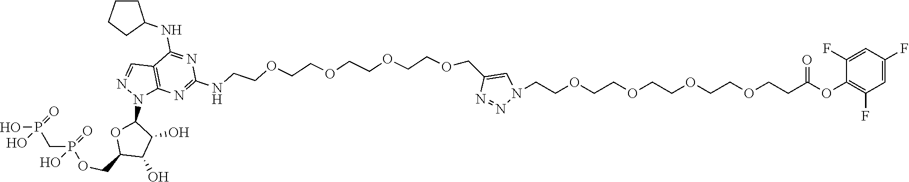

To a solution of the product from previous step (0.06 g, 0.03 mmol) and intermediate A (0.035 g, 0.083 mmol) dissolved in DMF:H₂O (1:3, 1.5 mL) was cooled to 0° C. To this a pre-mixed solution of a solution of copper(II) sulfate (0.0013 g, 0.0083 mmol), sodium ascorbate (0.049 g, 0.249 mmol), and BTTA (0.007 g, 0.0167 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). White solid (0.06 g, 63.17%). LCMS[(M+2H)/2]⁺=572.2.

### Synthesis of Conjugate 31

Trifluorophenol ester (described in the synthesis of int-55) was conjugated to Fc carrier SEQ ID NO: 13 as described in the synthesis of Conjugate 8a. Maldi TOF analysis of the purified final product gave an average mass of 61,874 Da (DAR=3.7). Yield 61.9 mg, 77.4%.

### Synthesis of Int-28

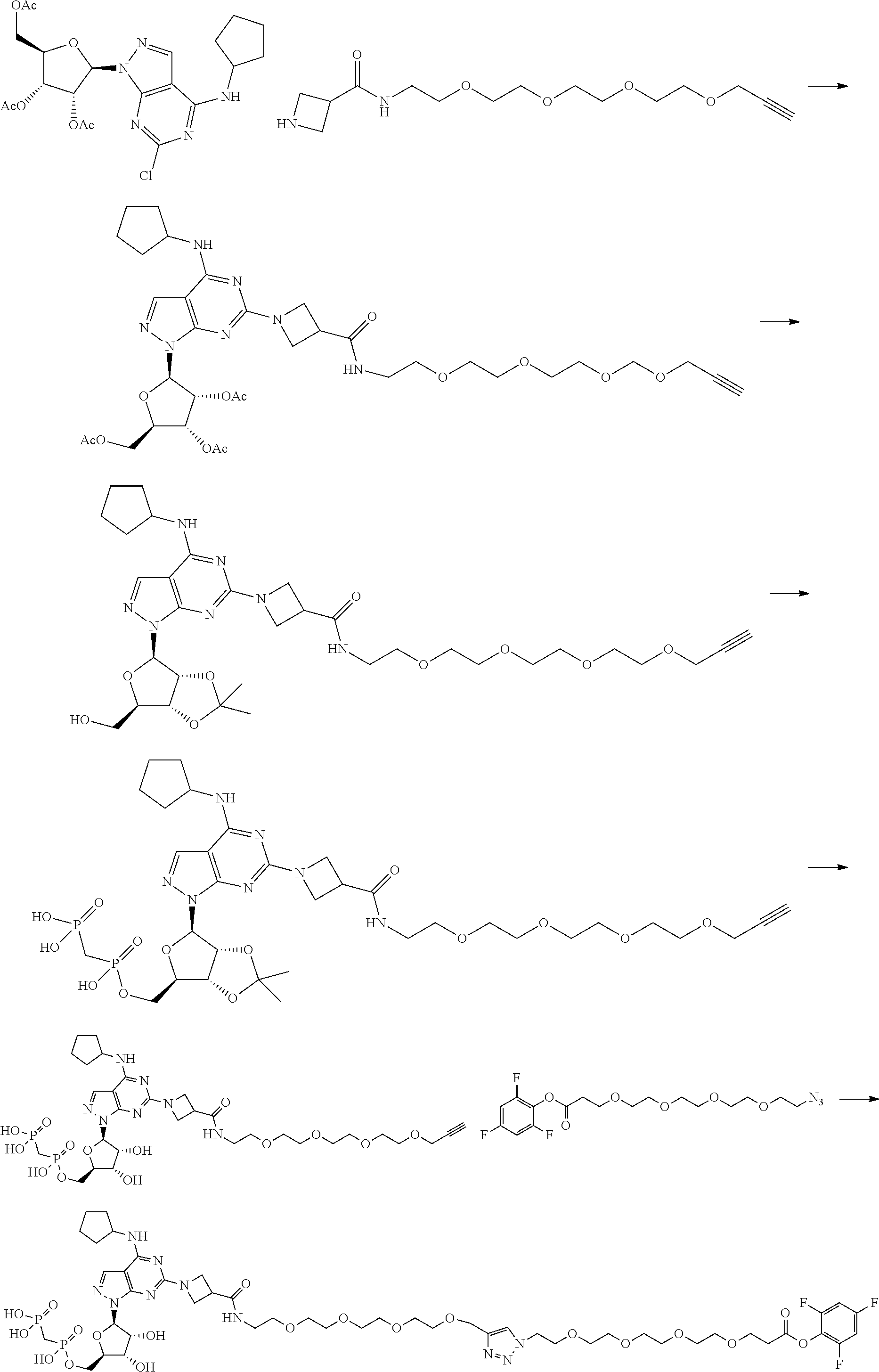

### Step a

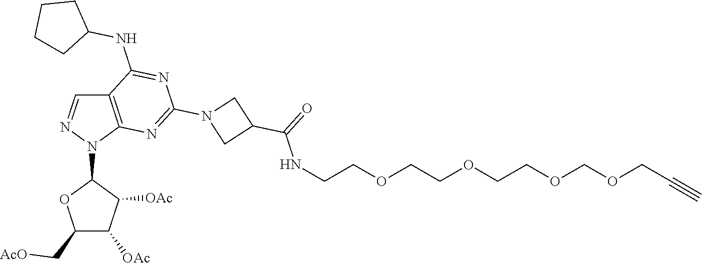

A mixture of pyrazolo-pyrimidine derivative described in step a of the synthesis of Int-55 (880 mg, 1.76 mmol), and the azetidine intermediate described in step a, of the synthesis of Int-10 (668 mg, 2.12 mmol) and triethylamine (0.17 mL) in ethanol (25 mL) were heated at 80° C. for 12 hours. The mixture was cooled to ambient temperature and concentrated under reduced pressure. The residue was purified by column chromatography (petroleum ether/ethyl acetate: 5:1) to provide the title compound. The product was a white solid. Yield 508 mg, 37%. Ions found by LCMS: [M+H]⁺=774.8.

### Step b

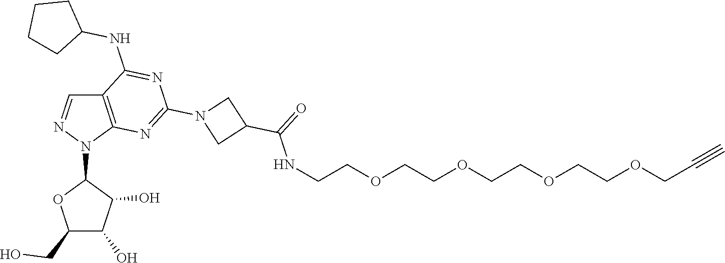

The triacetate from the previous step (412 mg, 0.53 mmol) was dissolved in methanol (10 mL). Potassium carbonate (257 mg, 1.86 mmol) was added, and the reaction mixture was stirred at ambient temperature for 1.5 hours. The reaction mixture was filtered through celite, and the filter cake was washed with methanol (3×20 mL). The solution was concentrated under reduced pressure to afford the crude triol which was purified by reversed phase HPLC to yield the desired compound. The product was a white solid. Yield: 310 mg, 89%. Ions found by LCMS: [M+H]⁺=648.8.

### Step c

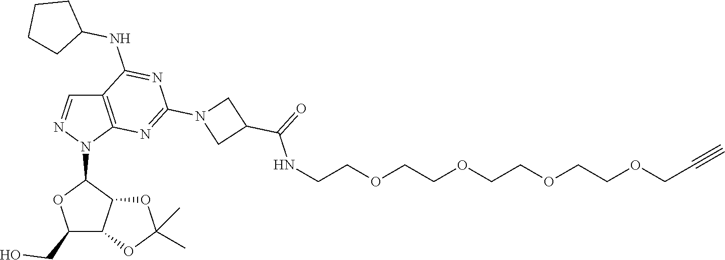

To a solution of the triol from the previous step (310 mg, 0.48 mmol) and 2,2-dimethoxypropane (0.29 mL, 2.39 mmol) in acetone (25 mL) at ambient temperature was added p-TsOH (8 mg, 0.048 mmol). The reaction was stirred for two hours then concentrated under reduced pressure. The crude product was dissolved in ethyl acetate (30 mL) and washed with saturated sodium bicarbonate. The organic layer was separated and dried over sodium sulfate, filtered, and concentrated in vacuo to provide crude material. The product was a white solid. Yield 329 mg, 99%. Ions found by LCMS: [M+H]⁺=688.6.

### Step d

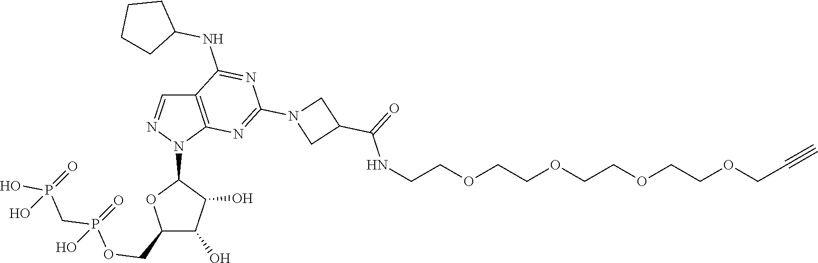

To a suspension of methylenebis(phosphonic dichloride) (359 mg, 1.44 mmol) in THE (5 mL) at 0° C. was added DIEA (0.092 mL, 0.182 mmol). The acetonide intermediate from the previous step (328 mg, 0.48 mmol) in THE (2 mL) was added to the mixture, dropwise over the course of 10 minutes. Following addition, the resulting mixture was stirred at 0° C. for an additional 15 minutes, then the solution was added dropwise to a pre-cooled (0° C.) flask containing 0.2 M aqueous HCl. The reaction mixture was warmed to ambient temperature and stirred for 4 hours. Upon completion (by LCMS), the reaction mixture was concentrated under reduced pressure and the crude material was purified by reversed phase HPLC (10% to 100% ACN/water, 0.1% TFA). The product was a white solid. Yield 246 mg, 64%. Ions found by LCMS: [M+H]⁺=806.6.

### Step e

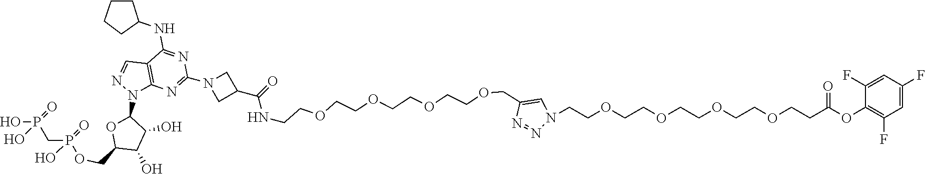

A solution of product from step d (0.06 g, 0.0745 mmol), and intermediate A (0.031 g, 0.0745 mmol) dissolved in DMF:H₂O (1:3, 1.5 mL) was cooled to 0° C. To this a pre-mixed solution of a solution of copper(II) sulfate (0.0012 g, 0.007446 mmol), sodium ascorbate (0.044 g, 0.2234 mmol), and BTTA (0.006 g, 0.0148 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). The product was a white solid. Yield 70 mg, 63.8%. Ions found by LCMS: [(M+2H)/2]⁺=613.6.

### Synthesis of Conjugate 17

Trifluorophenol ester (9 mg, 0.00724 mmol) described in the synthesis of Int-28 was conjugated to Fc carrier SEQ ID NO: 13 (50 mg, 2.58 mL in PBS at pH 7.4, 0.0009 mmol) as described in synthesis of Conjugate 8a. Maldi TOF analysis of the purified final product gave an average mass of 60,273 Da (DAR=1.9). Yield 43.9 mg, 87.7%.

### Synthesis of Int-49

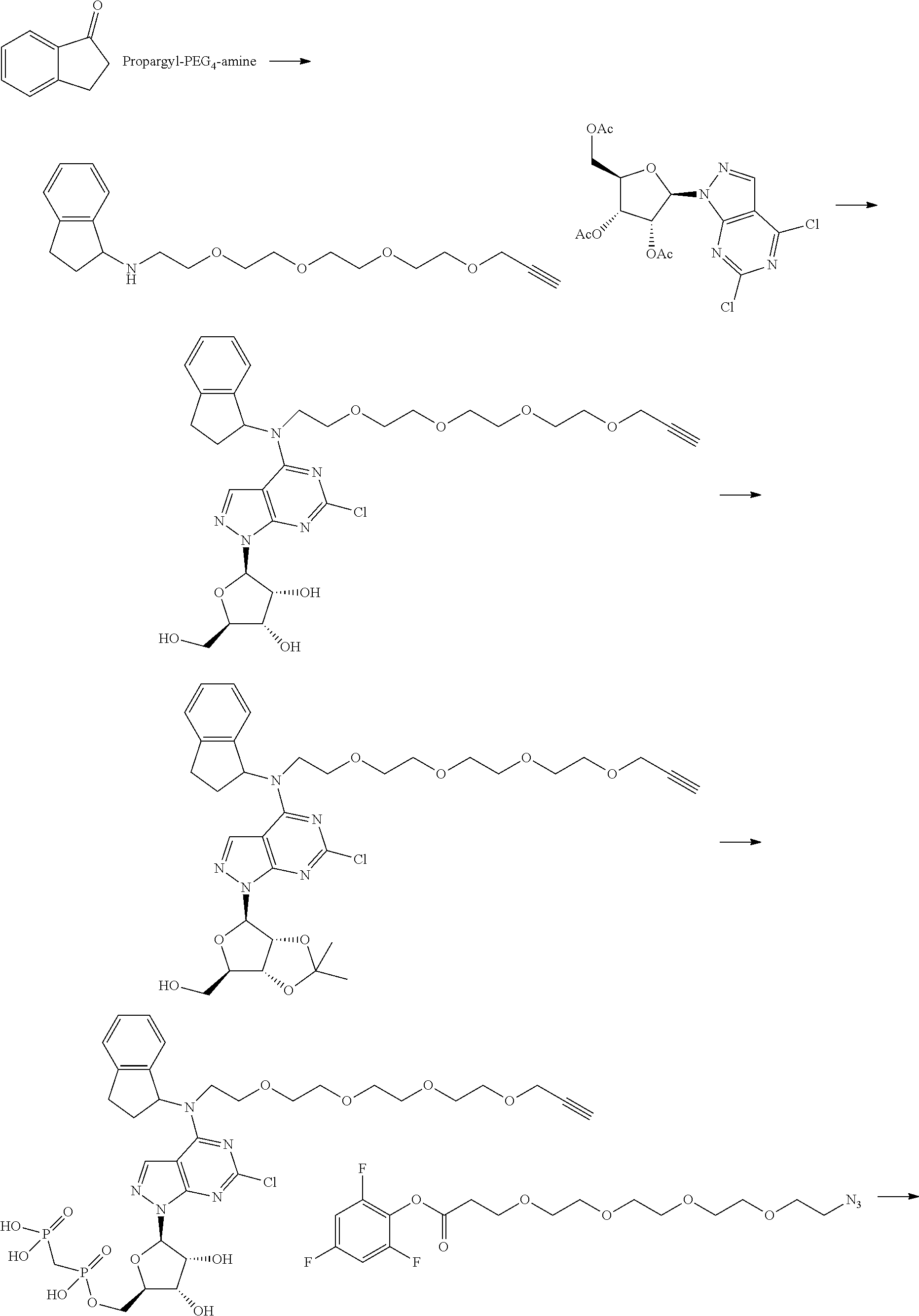

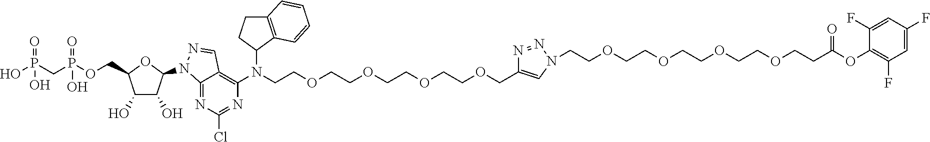 [SMILES: O=C(CCOCCOCCOCCOCCn1cc(COCCOCCOCCOCCN(c2nc(Cl)nc3c2cnn3[C@@H]2O[C@H](COP(=O)(O)CP(=O)(O)O)[C@@H](O)[C@H]2O)C2CCc3ccccc32)nn1)Oc1c(F)cc(F)cc1F]

### Step a

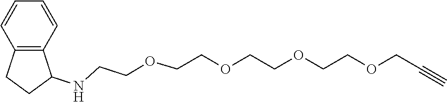 [SMILES: C#CCOCCOCCOCCOCCNC1CCc2ccccc21]

A mixture of 1-indanone (132 mg, I mmol), propargyl PEG₄-amine (277 mg, 1.2 mmol) and acetic acid (0.003 mL, 0.06 mol) in benzene (6.0 mL) were stirred under reflux for 4 hours. The reaction mixture was cooled under an atmosphere of nitrogen to 20° C., concentrated under reduced pressure to half volume. The residue was added to a stirred solution of sodium borohydride (19 mg, 0.5 mmol) in ethanol (5 mL) at ambient temperature. The mixture was stirred for 24 hours then quenched by the addition of water (˜3 mL). Solvents were evaporated under reduced pressure. The residue was extracted with ethyl acetate (3×10 mL). The combined organic extracts were dried over sodium sulfate, filtered and concentrated under reduced pressure to yield the amine intermediate as a dark brown viscous liquid. Yield 111 mg, 32%. Ions found by LCMS: [M+H]⁺=348.0.

### Step b

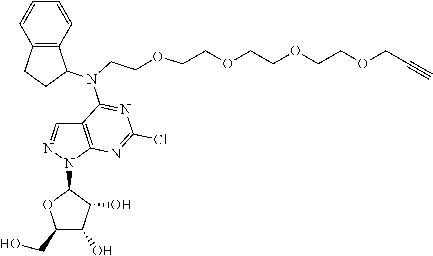 [SMILES: C#CCOCCOCCOCCOCCN(c1nc(Cl)nc2c1cnn2[C@@H]1O[C@H](CO)[C@@H](O)[C@H]1O)C1CCc2ccccc21]

A mixture of intermediate B (100 mg, 0.222 mmol), the crude amine from the previous step (93 mg, 0.268 mmol) and triethylamine (0.046 mL, 0.3354) in ethanol (5 mL) were heated at 50° C. for 1 hour. The mixture was cooled to ambient temperature and concentrated to give crude product as pale yellow, viscous oil. The crude material was used for the next step without any purifications. LCMS[M+H]⁺=758.6. The crude product was dissolved THE (5 mL) and 1M aqueous LiOH (1.34 mL, 1.34 mmol) was added and the resulting mixture was stirred for 30 minutes at ambient temperature. The mixture was acidified with 1 N aqueous HCl (to pH ˜4) and the solvent was removed under reduced pressure. The crude material was purified by reversed phase HPLC to yield the product as a light yellow solid. Yield 141 mg, 84%. Ions found by LCMS: 631.8 [M+H]⁺.

### Step c

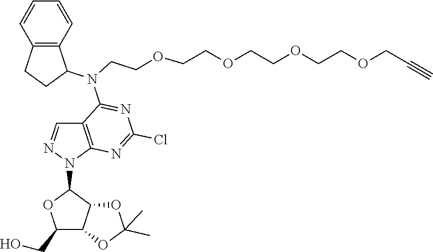 [SMILES: C#CCOCCOCCOCCOCCN(c1nc(Cl)nc2c1cnn2[C@@H]1O[C@H](CO)[C@H]2OC(C)(C)O[C@H]21)C1CCc2ccccc21]

To a solution of the product from the previous step (119 mg, 0.188 mmol) and 2,2-dimethoxypropane (0.11 mL, 0.94 mmol) in acetone (10 mL) at room temperature was added p-TsOH (3 mg). The reaction was stirred for two hours then concentrated under reduced pressure. The crude residue was dissolved in ethyl acetate (30 mL) and washed with saturated sodium bicarbonate. The organic layer was separated and dried over sodium sulfate, filtered and concentrated in vacuo to provide an off-white solid which was used for the next step without further purification. Yield 127 mg, 99%. Ions found by LCMS: [M+H]⁺=674.6.

### Step d

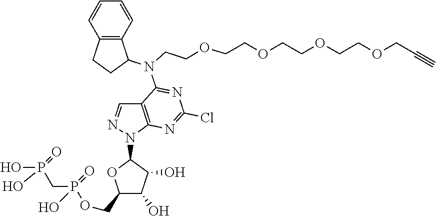 [SMILES: C#CCOCCOCCOCCOCCN(c1nc(Cl)nc2c1cnn2[C@@H]1O[C@H](COP(=O)(O)CP(=O)(O)O)[C@@H](O)[C@H]1O)C1CCc2ccccc21]

To a suspension of methylenebis(phosphonic dichloride) (141 mg, 0.56 mmol) in THE (5 mL) at 0° C. was added DIEA (0.036 mL, 0.21 mmol). To the resulting mixture was added to a solution of the product from the previous step (128 mg, 0.19 mmol) in THE (2 mL) dropwise over the course of 10 minutes. Following addition, the resulting mixture was stirred at 0° C. for an additional 15 minutes, then the solution was added dropwise to a pre-cooled (0° C.) flask containing 0.2 aqueous M HCl. The reaction mixture was warmed to ambient temperature and stirred for 4 hours. Upon completion (by LCMS), the reaction mixture was concentrated under reduced pressure and the crude material was purified by reversed phase HPLC (10% to 100% ACN/water, 0.1% TFA). The product was a yellow solid 34 mg, 23%. Ions found by LCMS: [M+H]⁺=790.2.

### Step e

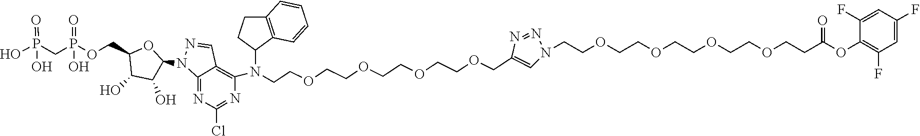 [SMILES: O=C(CCOCCOCCOCCOCCn1cc(COCCOCCOCCOCCN(c2nc(Cl)nc3c2cnn3[C@@H]2O[C@H](COP(=O)(O)CP(=O)(O)O)[C@@H](O)[C@H]2O)C2CCc3ccccc32)nn1)Oc1c(F)cc(F)cc1F]

A solution of product from the previous step d (0.03 g, 0.03797 mmol), and intermediate A (0.016 g, 0.03797 mmol) dissolved in DMF:H₂O (1:3, 1.5 mL) was cooled to ° C. To this a pre-mixed solution of a solution of copper(II) sulfate (0.0006 g, 0.003797 mmol), sodium ascorbate (0.032 g, 0.1139 mmol), and BTTA (0.003 g, 0.007 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). White solid (0.016 g, 34.78%). LCMS[(M+H)/2]⁺=605.8.

### Synthesis of Conjugate 28

Trifluorophenol ester (7 mg, 0.0058 mmol) described in the synthesis of Int-49) was conjugated to Fc carrier SEQ ID NO: 13 (40 mg, 2 mL in PBS at pH 7.4, 0.000724 mmol) as described in synthesis of Conjugate 8a. Maldi TOF analysis of the purified final product gave an average mass of 64,083 Da (DAR=5.5). Yield 30.3 mg, 75.6%.

### Synthesis of Int-81

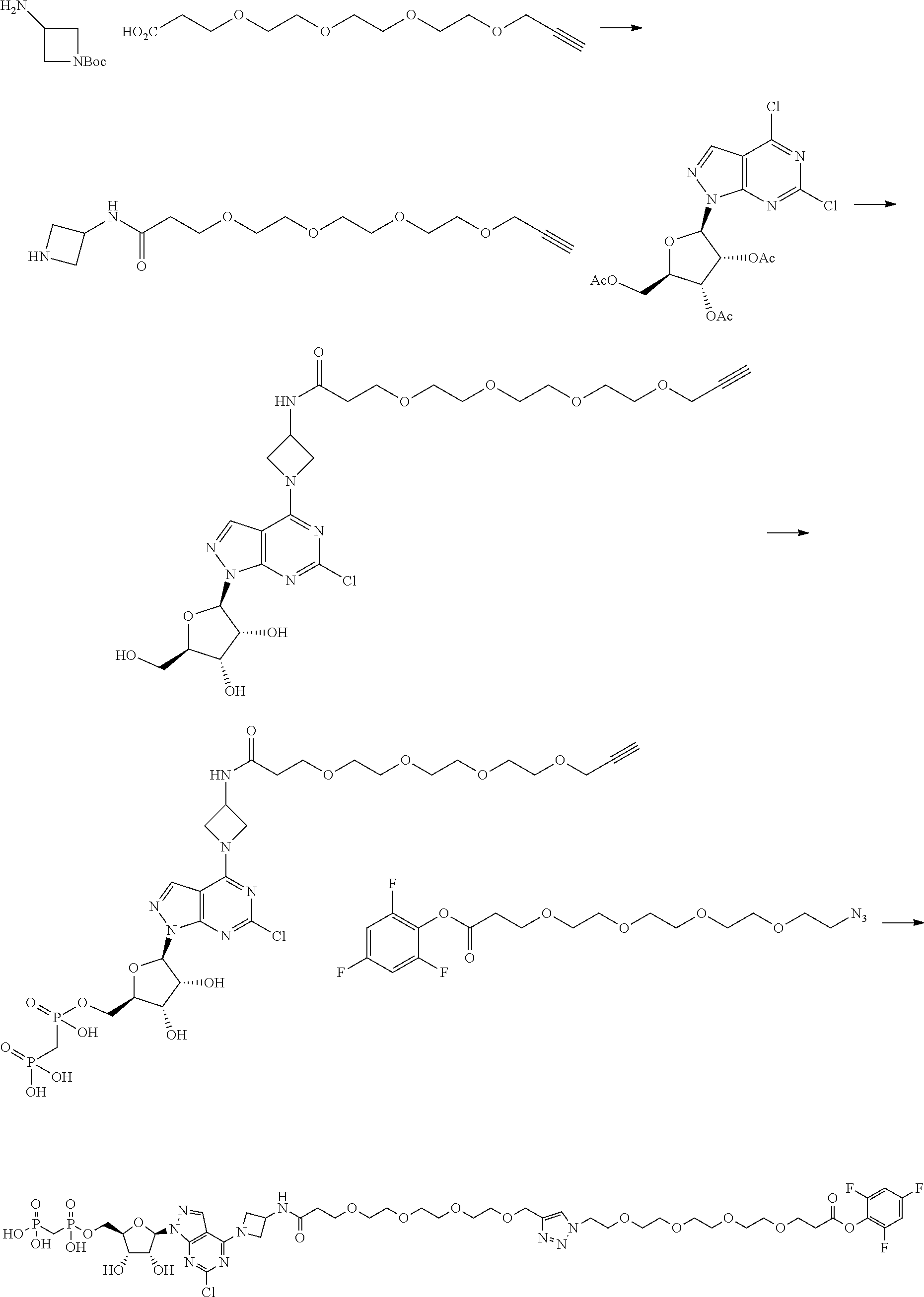

### Step a

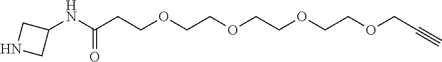 [SMILES: C#CCOCCOCCOCCOCCC(=O)NC1CNC1]

Propargyl-PEG₄-acid (544 mg, 2.09 mmol), HATU (662 mg, 1.74 mmol) and DIEA (0.62 mL, 3.48 mmol) in DMF (6 mL) were stirred together for 10 minutes at ambient temperature. Tert-butyl 3-amino-azetidinecarboxylate (300 mg, 1.74 mmol) in DMF (1 mL) was added and the resulting mixture was stirred for 1 hour at ambient temperature. The reaction mixture was concentrated under reduced pressure then the crude residue was purified by reversed phase HPLC (5% to 100% ACN/water) to afford the boc-protected azetidine intermediate as a yellow viscous oil. The boc-protected intermediate (578 mg, 1.39 mmol) was dissolved in dioxane (10 mL) and treated with 4M aqueous HCl in dioxane (7 mL) for 3 hours at ambient temperature. The solvent was removed under reduced pressure and dried under high vacuum to afford the azetidine as an HCl salt. The product was a yellow viscous oil. Yield 480 mg, 98%. Ions found by LCMS: [M+H]⁺=315.0.

### Step b

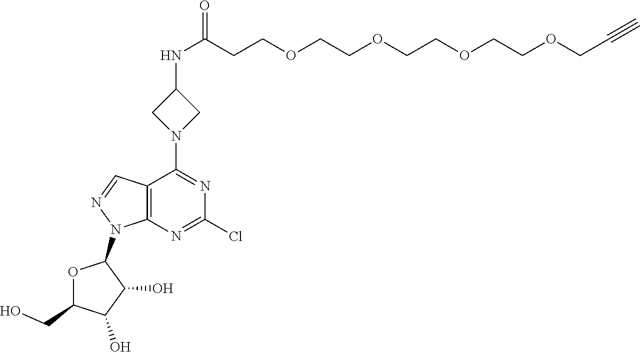

A mixture of intermediate B (250 mg, 0.56 mmol), the azetidine from the previous step (235 mg, 0.67 mmol), and triethylamine (0.15 mL, 1.12 mmol) in ethanol (6 mL, 1.12 mmol) were heated at 50° C. for 1 hour. The mixture was cooled to ambient temperature and concentrated on a rotary evaporator. The crude residue was dissolved in ethyl acetate (50 mL) and washed with water and brine. The organic layer was dried over sodium sulfate, filtered, and concentrated to afford the triacetate intermediate as a colorless viscous oil. Ions found by LCMS: [M+H]⁺=724.6. The crude triacetate (380 mg, 0.94 mmol) was dissolved methanol (5 mL), potassium carbonate (253 mg, 1.83 mmol) was added, and the mixture was stirred at ambient temperature for 2 hours. The mixture was concentrated under reduced pressure and purified by silica gel chromatography using 0% to 10% MeOH in DCM. The product was a white solid. Yield 280 mg, 89%. Ions found by LCMS: [M+H]⁺=598.8.

### Step c

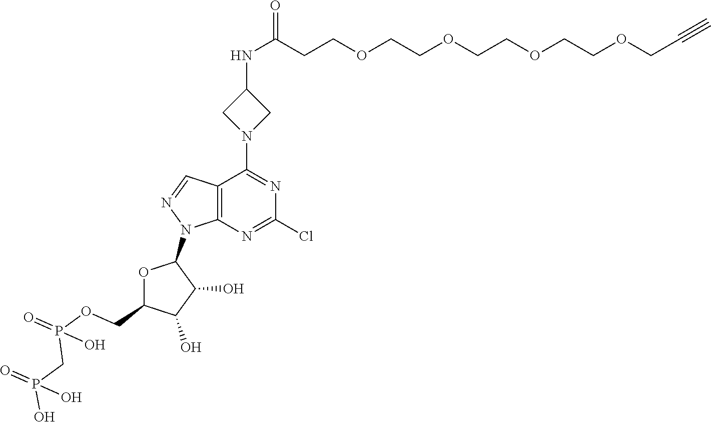

The product from the previous step (130 mg, 0.472 mmol) and 2,2-dimethoxypropane (0.28 mL, 2.34 mmol) in acetone (20 mL) at room temperature was added p-TsOH (8 mg, 0.005 mmol). The reaction was stirred for two hours then concentrated under reduced pressure. The crude material was dissolved in ethyl acetate (20 mL) and washed with saturated sodium bicarbonate. The organic layer was separated and dried over sodium sulfate, filtered, and concentrated under reduced pressure to provide the acetonide derivative. The product was a white solid. Ions found by LCMS: [M+H]⁺=639.8. To a suspension of methylenebis(phosphonic dichloride) (350 mg, 1.40 mmol) in THE (5 mL) at 0° C. was added DIEA (0.089 mL, 0.51 mmol). To the resulting mixture was added a solution of the acetonide derivative (298 g, 0.47 mmol) in THE (2 mL) dropwise over the course of 10 minutes. Following addition, the resulting mixture was stirred at 0° C. for an additional 15 minutes, then the solution was transferred to a pre-cooled (0° C.) flask containing 0.2 M aqueous HCl (2.5 mL). The reaction mixture was warmed to ambient temperature and stirred for 4 hours. Upon completion (by LCMS), the mixture was concentrated under reduced pressure and purified by reversed phase HPLC. The product was a white solid. Yield 220 mg, 62%. Ions found by LCMS: [M+H]⁺=757.8.

### Step d

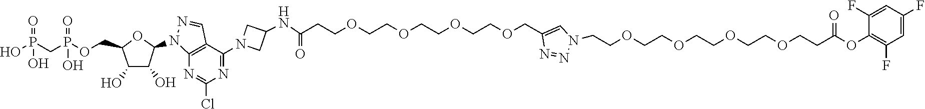 [SMILES: O=C(CCOCCOCCOCCOCc1cn(CCOCCOCCOCCOCCC(=O)Oc2c(F)cc(F)cc2F)nn1)NC1CN(c2nc(Cl)nc3c2cnn3[C@@H]2O[C@H](COP(=O)(O)CP(=O)(O)O)[C@@H](O)[C@H]2O)C1]

A solution of product from the previous step (0.05 g, 0.066 mmol), and intermediate A (0.027 g, 0.066 mmol) dissolved in DMF:H₂O (1:3, 1.5 mL) was cooled to ° C. To this a pre-mixed solution of a solution of copper(II) sulfate (0.0001 g, 0.006 mmol), sodium ascorbate (0.039 g, 0.198 mmol), and BTTA (0.006 g, 0.013 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). The product was a white solid. Yield 48 mg, 62%. Ions found by LCMS: [M+H]⁺=1179.4.

### Synthesis of Conjugate 44

Trifluorophenol ester (14 mg, 0.011584 mmol) described in the synthesis of Int-81) was conjugated to Fc carrier SEQ ID NO: 13 (80 mg, 4.1 mL in PBS at pH 7.4, 0.00145 mmol) 5 as described in synthesis of Conjugate 8a Maldi TOF analysis of the purified final product gave an average mass of 63,709 Da (DAR=5.3). Yield 63 mg, 78.9%.

### Synthesis of Int-90

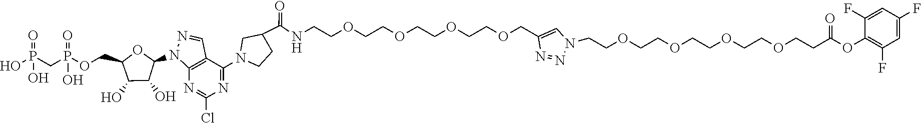 [SMILES: O=C(CCOCCOCCOCCOCCn1cc(COCCOCCOCCOCCNC(=O)C2CCN(c3nc(Cl)nc4c3cnn4[C@@H]3O[C@H](COP(=O)(O)CP(=O)(O)O)[C@@H](O)[C@H]3O)C2)nn1)Oc1c(F)cc(F)cc1F]

The title compound was prepared analogously to Int-10 where N-Boc azetidine-3-carboxylic acid was replaced with 1-[(tert-Butyl)oxycarbonyl]pyrrolidine-3-carboxylic acid. The product was a white solid. Yield 52 mg, 67%. Ions found by LCMS: [(M+2H)/2]⁺=596.8.

### Synthesis of Conjugate 48

Trifluorophenol ester (17 mg, 0.01448 mmol) described in the synthesis of Int-90) was conjugated to Fc carrier SEQ ID NO: 13 (100 mg, 5.15 mL, 0.0018 mmol) in PBS at pH 7.4 as described in synthesis of Conjugate 8a. Maldi TOF analysis of the purified final product gave an average mass of 63,468 Da (DAR=5). Yield 53.2 mg, 53.2%.

### Synthesis of Int-91

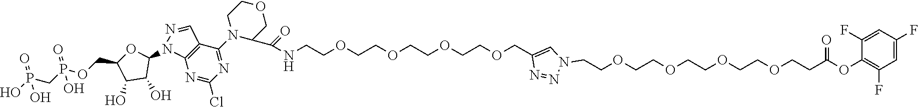 [SMILES: O=C(CCOCCOCCOCCOCCn1cc(COCCOCCOCCOCCNC(=O)C2COCCN2c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

The title compound was prepared analogously to Int-10 where N-Boc azetidine-3-carboxylic acid was replaced with 4-[(tert-butyl)oxycarbonyl]morpholine-3-carboxylic acid. The product was a white solid. Yield 68 mg, 55%. Ions found by LCMS: [(M+2H)/2]⁺=605.2.

### Synthesis of Conjugate 49

Trifluorophenol ester (17.5 mg, 0.01448 mmol) described in the synthesis of Int-91 was conjugated to Fc carrier SEQ ID NO: 13 (100 mg, 5.15 mL in PBS at pH 7.4, 0.0018 mmol) as described in the synthesis of Conjugate 8a. Maldi TOF analysis of the purified final product gave an average mass of 64,352 Da. (DAR=5.8). Yield 70 mg, 70%.

### Synthesis of Int-97

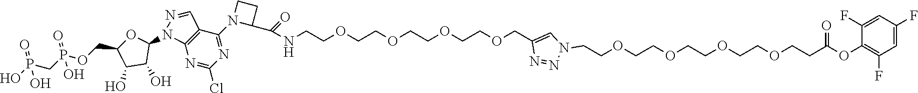 [SMILES: O=C(CCOCCOCCOCCOCCn1cc(COCCOCCOCCOCCNC(=O)C2CCN2c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

The title compound was prepared analogously to step e of the synthesis of Int-10 where N-Boc azetidine-3-carboxylic acid was replaced with N-Boc azetidine-2-carboxylic acid. The product was a white solid. Yield 56 mg, 72%. Ions found by LCMS: [M+H]⁺=1179.4.

### Synthesis of Conjugate 52

Trifluorophenol ester (17 mg, 0.01448 mmol) described in the synthesis of Int-97 was conjugated to Fc carrier SEQ ID NO: 13 (100 mg, 5.15 mL, 0.0018 mmol) in PBS at pH 7.4 as described in synthesis of Conjugate 8a. Maldi TOF analysis of the purified final product gave an average mass of 63,232 Da (DAR=4.9). Yield 73 mg, 73%.

### Synthesis of Int-99

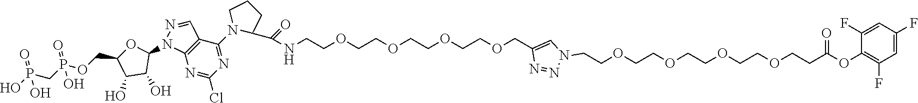 [SMILES: O=C(CCOCCOCCOCCOCCn1cc(COCCOCCOCCOCCNC(=O)C2CCCN2c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

The title compound was prepared analogously to Int-10 where N-Boc azetidine-3-carboxylic acid was replaced with N-Boc-proline. The product was a white solid. Yield 31 mg, 40.1%. Ions found by LCMS: [(M+2H)/2]⁺=596.8.

### Synthesis of Conjugate 53

Trifluorophenol ester (17 mg, 0.01448 mmol) described in the synthesis of Int-99 was conjugated to Fc carrier SEQ ID NO: 13 (100 mg, 5.15 mL, 0.0018 mmol) in PBS at pH 7.4 as described in synthesis of Conjugate 8a. Maldi TOF analysis of the purified final product gave an average mass of 61,730 Da (DAR 3.4). Yield 70 mg, 70%.

### Synthesis of Int-113

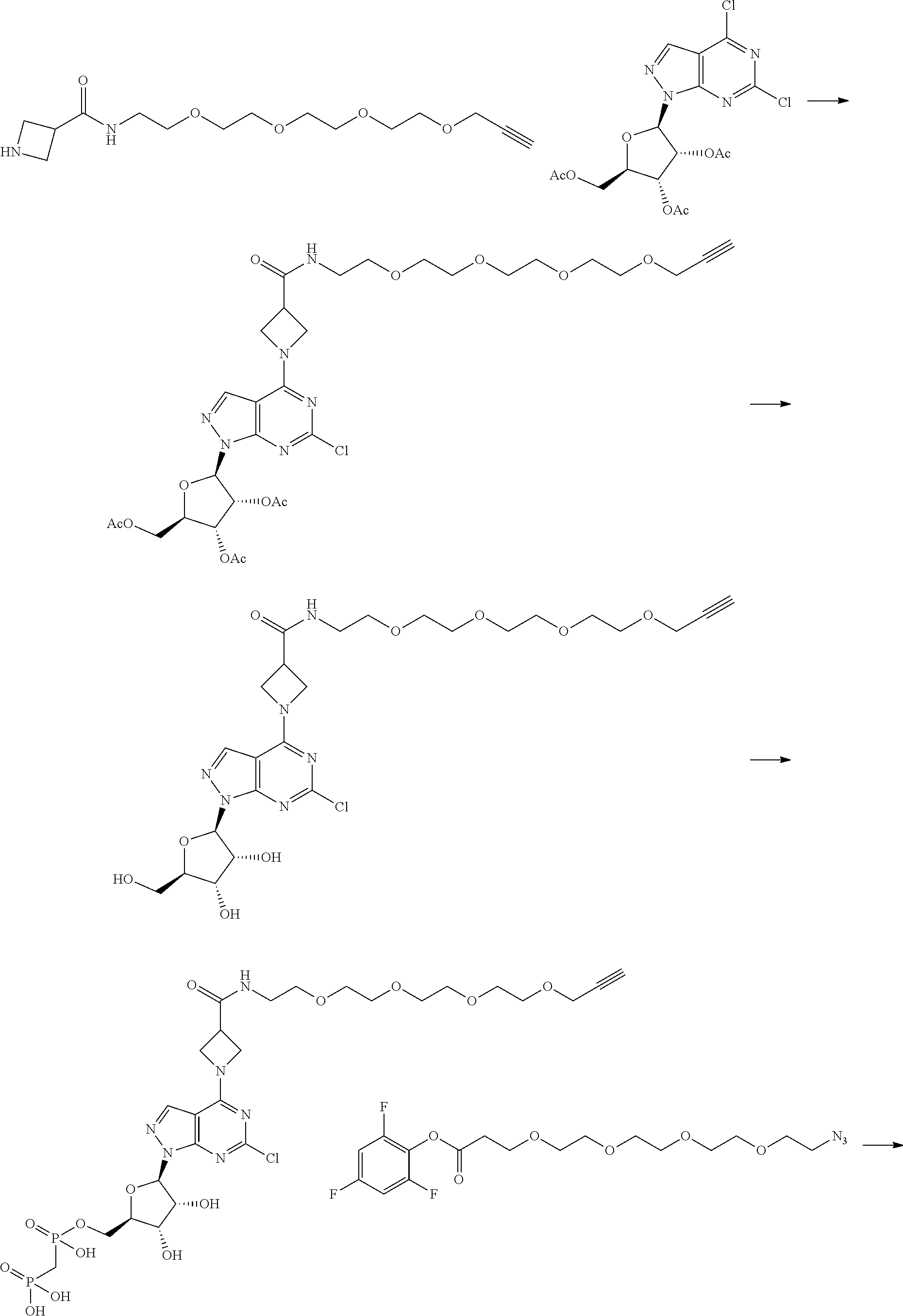 [SMILES: C#CCOCCOCCOCCOCCNC(=O)C1CNC1 ; CC(=O)OC[C@H]1O[C@@H](n2ncc3c(Cl)nc(Cl)nc32)[C@H](OC(C)=O)[C@@H]1OC(C)=O ; C#CCOCCOCCOCCOCCNC(=O)C1CN(c2nc(Cl)nc3c2cnn3[C@@H]2O[C@H](COC(C)=O)[C@@H](OC(C)=O)[C@H]2OC(C)=O)C1 ; C#CCOCCOCCOCCOCCNC(=O)C1CN(c2nc(Cl)nc3c2cnn3[C@@H]2O[C@H](CO)[C@@H](O)[C@H]2O)C1 ; C#CCOCCOCCOCCOCCNC(=O)C1CN(c2nc(Cl)nc3c2cnn3[C@@H]2O[C@H](COP(=O)(O)CP(=O)(O)O)[C@@H](O)[C@H]2O)C1 ; [N-]=[N+]=NCCOCCOCCOCCOCCC(=O)Oc1c(F)cc(F)cc1F]

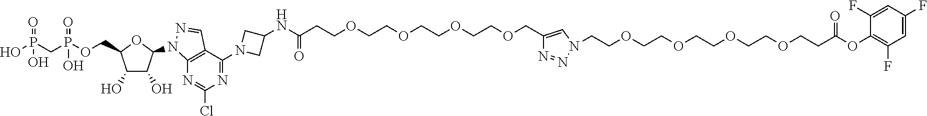 [SMILES: O=C(CCOCCOCCOCCOCc1cn(CCOCCOCCOCCOCCC(=O)Oc2c(F)cc(F)cc2F)nn1)NC1CN(c2nc(Cl)nc3c2cnn3[C@@H]2O[C@H](COP(=O)(O)CP(=O)(O)O)[C@@H](O)[C@H]2O)C1]

### Step a

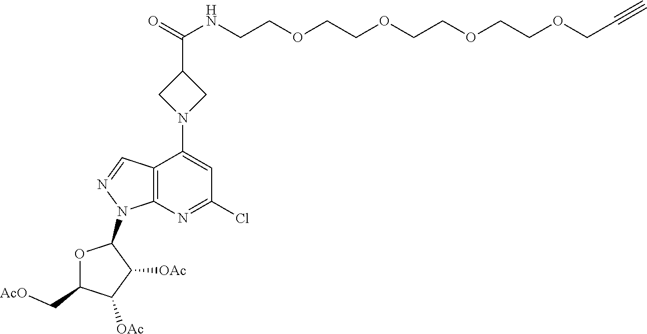 [SMILES: C#CCOCCOCCOCCOCCNC(=O)C1CN(c2cc(Cl)nc3c2cnn3[C@@H]2O[C@H](COC(C)=O)[C@@H](OC(C)=O)[C@H]2OC(C)=O)C1]

A mixture of intermediate C (200 mg, 0.448 mmol) and the azetidine intermediate described in step a of the synthesis of Int-10 (188 mg, 0.54 mmol), triethylamine (0.13 mL) in ethanol (6 mL) was heated at 110° C. for 12 hours. Upon consumption of the starting materials (by LCMS), the reaction mixture was cooled to ambient temperature and volatiles were removed under reduced pressure to yield the crude material which was used for the next step without further purification. Yellow viscous oil. Yield 324 mg, 100%. Ions found by LCMS: [M+H]⁺=724.6.

### Step b

The triacetate from the previous step (324 mg, 0.45 mmol) in methanol (5 mL) was treated with potassium carbonate (217 mg, 1.57 mmol) at ambient temperature for 2 hours. The reaction mixture was concentrated under reduced pressure, dissolved methanol:water (1:1, 2 mL) and purified by reversed phase HPLC (10% to 100% ACN/water, 0.1% TFA). White foam. Yield 210 mg, 78%. Ions found by LCMS: [M+H]⁺=598.2.

### Step c

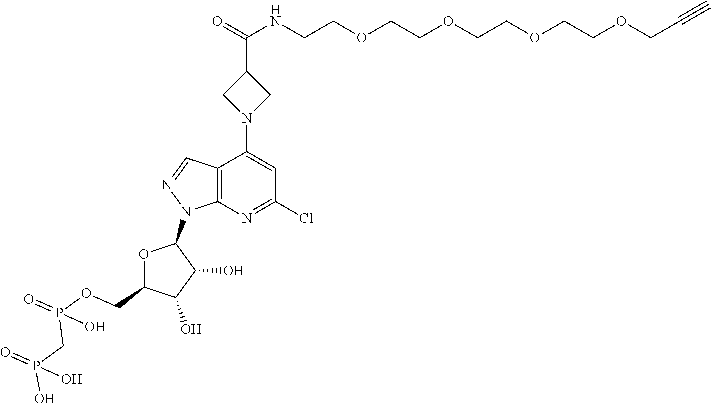

To a solution of the product from previous step (190 mg, 0.32 mmol) and 2,2-dimethoxypropane (0.19 mL, 1.58 mmol) in acetone (5 mL) at ambient temperature was added p-TsOH (6 mg, 0.032 mmol).

The reaction was stirred for two hours then concentrated under reduced pressure. The crude material was dissolved in ethyl acetate (20 mL) and washed with saturated sodium bicarbonate. The organic layer was separated and dried over sodium sulfate, filtered and concentrated under reduced pressure to provide the acetonide derivative. The product was a white solid. LCMS[M+H]⁺=638.2. To a suspension of methylenebis(phosphonic dichloride) (238 mg, 0.95 mmol) in THE (5 mL) at 0° C. was added DIEA (0.061 mL, 0.35 mmol). To the resulting mixture was added a solution of the acetonide derivative in THE (2 mL), dropwise over the course of 10 minutes. Following addition, the resulting mixture was stirred at 0° C. for 15 minutes, then the solution was transferred to a pre-cooled (0° C.) flask containing 0.2 M aqueous HCl (7 mL). The reaction mixture was warmed to ambient temperature and stirred for 4 hours. Upon completion (by LCMS), the reaction mixture was concentrated and purified by reversed phase HPLC. The product was a white solid. Yield 130 mg, 54%. Ions found by LCMS: [M+H]⁺=756.2.

### Step d

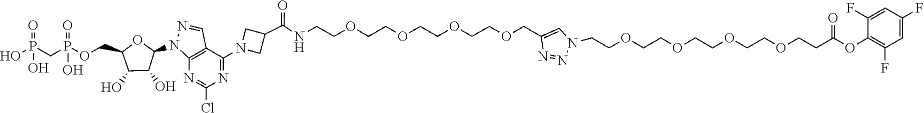 [SMILES: O=C(CCOCCOCCOCCOCCn1cc(COCCOCCOCCOCCNC(=O)C2CN(c3nc(Cl)nc4c3cnn4[C@@H]3O[C@H](COP(=O)(O)CP(=O)(O)O)[C@@H](O)[C@H]3O)C2)nn1)Oc1c(F)cc(F)cc1F]

A solution of product from the previous step (0.03 g, 0.039 mmol), and intermediate A (0.017 g, 0.0.039 mmol) dissolved in DMF:H₂O (1:3, 1.5 mL) was cooled to 0° C. To this a pre-mixed solution of a solution of copper(II) sulfate (0.00006 g, 0.0.0004 mmol), sodium ascorbate (0.024 g, 0.118 mmol), and BTTA (0.0064 g, 0.008 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). White solid (0.016 g, 34.25%). LCMS[(M+2H))/2]⁺=589.6 Synthesis of Conjugate 60 Trifluorophenol ester (18 mg, 0.01448 mmol) (described in the synthesis of Int-113) in DMF (0.5 mL) was added to Fc carrier SEQ ID NO: 13 (100 mg in 5.15 mL PBS at pH 7.4) then adjusted to pH-7.4 with sodium carbonate buffer (0.200 mL, pH 9.2 to 10.6, 0.1 M). The mixture was agitated at room temperature for 4 hours. The reaction mixture was quenched by adding a 150 mM Histidine/100 mM ammonium hydroxide buffer, pH 8.5, (˜0.5 mL of buffer mixture/10 mg of protein) and stirred for 12 hours then purified according to the general procedure. Maldi TOF analysis of the purified final product gave an average mass of 61,118 Da. (DAR of 2.9). Yield 54.9 mg, 61%.

### Synthesis of Int-115

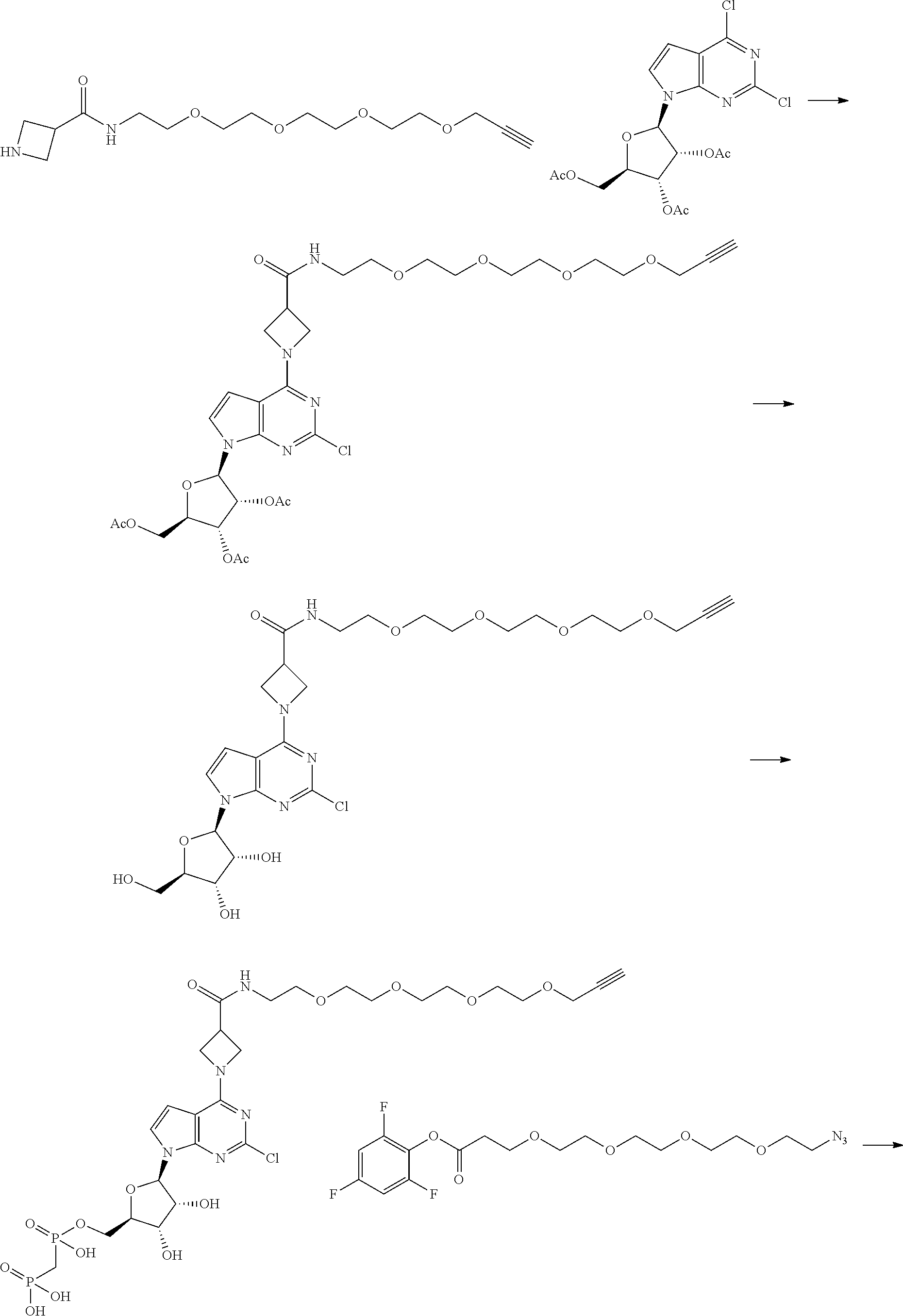 [SMILES: C#CCOCCOCCOCCOCCNC(=O)C1CNC1 ; CC(=O)OC[C@H]1O[C@@H](n2ccc3c(Cl)nc(Cl)nc32)[C@H](OC(C)=O)[C@@H]1OC(C)=O ; C#CCOCCOCCOCCOCCNC(=O)C1CN(c2nc(Cl)nc3c2ccn3[C@@H]2O[C@H](COC(C)=O)[C@@H](OC(C)=O)[C@H]2OC(C)=O)C1 ; C#CCOCCOCCOCCOCCNC(=O)C1CN(c2nc(Cl)nc3c2ccn3[C@@H]2O[C@H](CO)[C@@H](O)[C@H]2O)C1 ; C#CCOCCOCCOCCOCCNC(=O)C1CN(c2nc(Cl)nc3c2ccn3[C@@H]2O[C@H](COP(=O)(O)CP(=O)(O)O)[C@@H](O)[C@H]2O)C1 ; [N-]=[N+]=NCCOCCOCCOCCOCCC(=O)Oc1c(F)cc(F)cc1F]

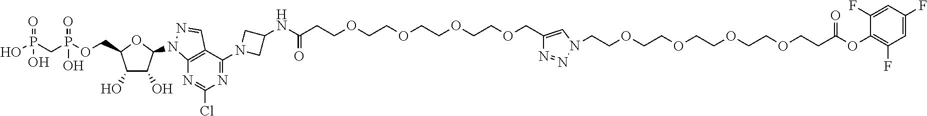 [SMILES: O=C(CCOCCOCCOCCOCc1cn(CCOCCOCCOCCOCCC(=O)Oc2c(F)cc(F)cc2F)nn1)NC1CN(c2nc(Cl)nc3c2cnn3[C@@H]2O[C@H](COP(=O)(O)CP(=O)(O)O)[C@@H](O)[C@H]2O)C1]

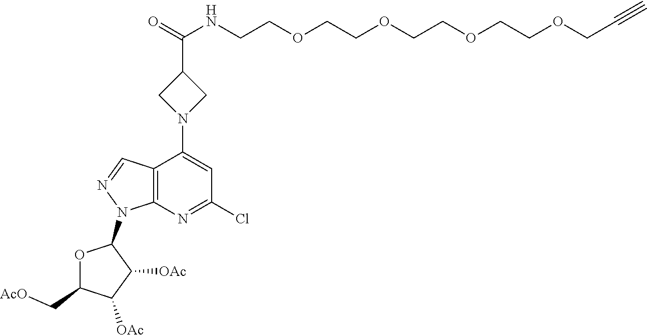

A mixture of intermediate D (250 mg, 0.56 mmol) and the azetidine intermediate from step a of example synthesis of Int-10 (235 mg, 0.67 mmol) and triethylamine (0.16 mL) in ethanol (6 mL) were heated at 50° C. for 1 hour. When the reaction was complete (by LCMS), the mixture was cooled to ambient temperature and removal of solvent under reduced pressure gave the crude material which was purified by silica gel column chromatography using hexanes: ethyl acetate. The product was a white solid. Yield 336 mg, 83%. Ions found by LCMS: [M+H]⁺=724.2.

### Step b

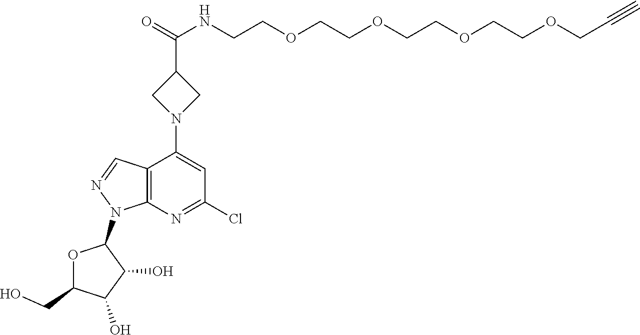

The triacetate from the previous step (336 mg, 0.47 mmol) in methanol (5 mL) was treated with potassium carbonate (224 mg, 1.56 mmol) at room temperature for 2 hours, the reaction mixture was concentrated in vacuo to remove volatiles and then re-dissolved in methanol:water (1:1, 2 mL). The crude mixture was purified by reversed phase HPLC (10% to 100% ACN/water, 0.1% TFA). The product was a white foam. Yield 190 mg, 69%. Ions found by LCMS: [M+H]⁺=598.2.

### Step c

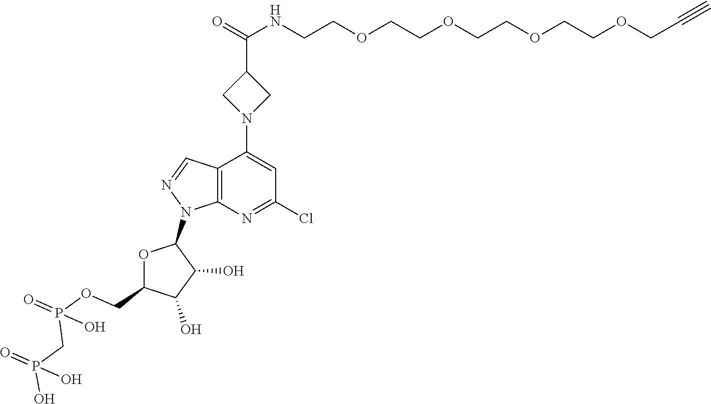

To a solution of the product from previous step (160 mg, 0.27 mmol) and 2,2-dimethoxypropane (0.163 mL, 1.34 mmol) in acetone (5 mL) at room temperature was added p-TsOH (5 mg, 0.027 mmol). The reaction was stirred for two hours then concentrated under reduced pressure. The crude material was dissolved in ethyl acetate (20 mL) and washed with saturated sodium bicarbonate. The organic layer was separated and dried over sodium sulfate, filtered, and concentrated under reduced pressure to provide the acetonide intermediate. The product was a white solid. Ions found by LCMS: [M+H]⁺=638.2. To a suspension of methylenebis(phosphonic dichloride) (200 mg, 0.80 mmol) in THE (5 mL) at 0° C. was added DIEA (0.051 mL, 0.29 mmol). To the resulting mixture was added a solution of the acetonide derivative in THE (2 mL) dropwise over the course of 10 minutes. Following addition, the resulting mixture was stirred at 0° C. for an additional 15 minutes, then the solution was transferred to a pre-cooled (0° C.) flask containing 0.2 M aqueous HCl (6 mL). The reaction mixture was warmed to ambient temperature and stirred for 4 hours. Upon completion (by LCMS), the reaction mixture was concentrated purified by reversed phase HPLC. The product was a white solid. Yield 164 mg, 91%. Ions found by LCMS: [M+H]⁺=756.2.

### Step d

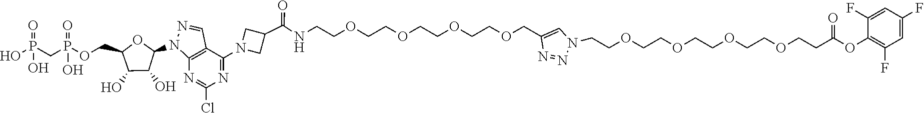 [SMILES: O=C(CCOCCOCCOCCOCCn1cc(COCCOCCOCCOCCNC(=O)C2CN(c3nc(Cl)nc4c3cnn4[C@@H]3O[C@H](COP(=O)(O)CP(=O)(O)O)[C@@H](O)[C@H]3O)C2)nn1)Oc1c(F)cc(F)cc1F]

A solution of product from the previous step (0.06 g, 0.079 mmol), and Intermediate A (0.033 g, 0.079 mmol) dissolved in DMF:H₂O (1:3, 1.5 mL) was cooled to 0° C. To this a pre-mixed solution of a solution of copper(II) sulfate (0.001 g, 0.0079 mmol), sodium ascorbate (0.047 g, 0.238 mmol), and BTTA (0.007 g, 0.016 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). The product was a white solid. Yield 50 mg, 53.5%. Ions found by LCMS: [M+H]⁺=1177.2.

### Synthesis of Conjugate 61

Trifluorophenol ester (18 mg, 0.01448 mmol) (described in the synthesis of Int-115) in DMF (0.5 mL) was added to Fc carrier SEQ ID NO: 13 (100 mg in 5.15 mL PBS at pH 7.4) 205 then adjusted to pH-7.4 with sodium carbonate buffer (0.200 mL, pH 9.2 to 10.6, 0.1 M). The mixture was agitated at ambient temperature for 4 hours. The reaction mixture was quenched by adding a 150 mM Histidine/100 mM ammonium hydroxide buffer, pH 8.5, (˜0.5 mL of buffer mixture/10 mg of protein) and stirring for 12 hours then submitted for purification according to the general procedure. Maldi TOF analysis of the purified final product gave an average mass of 61,737 Da (DAR of 3.4). Yield 59.9 mg, 59.9%.

### Synthesis of Int-117

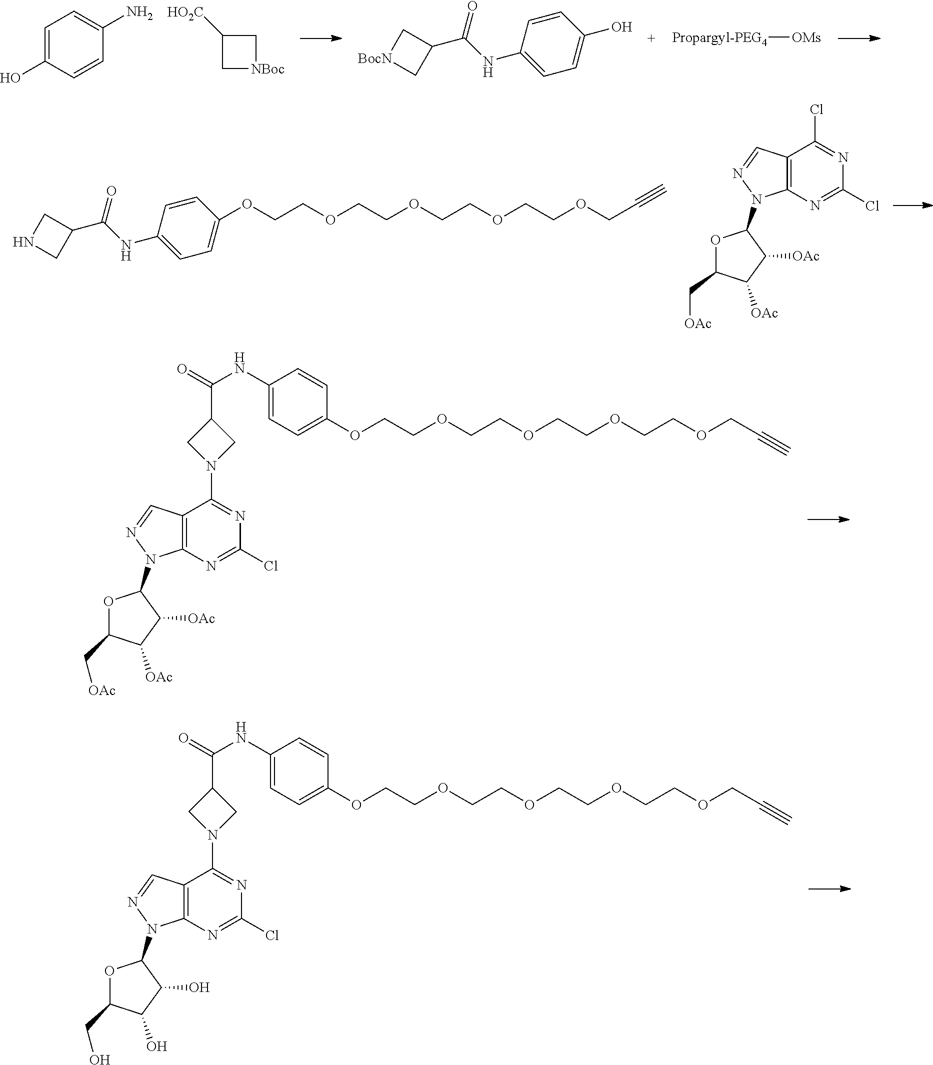

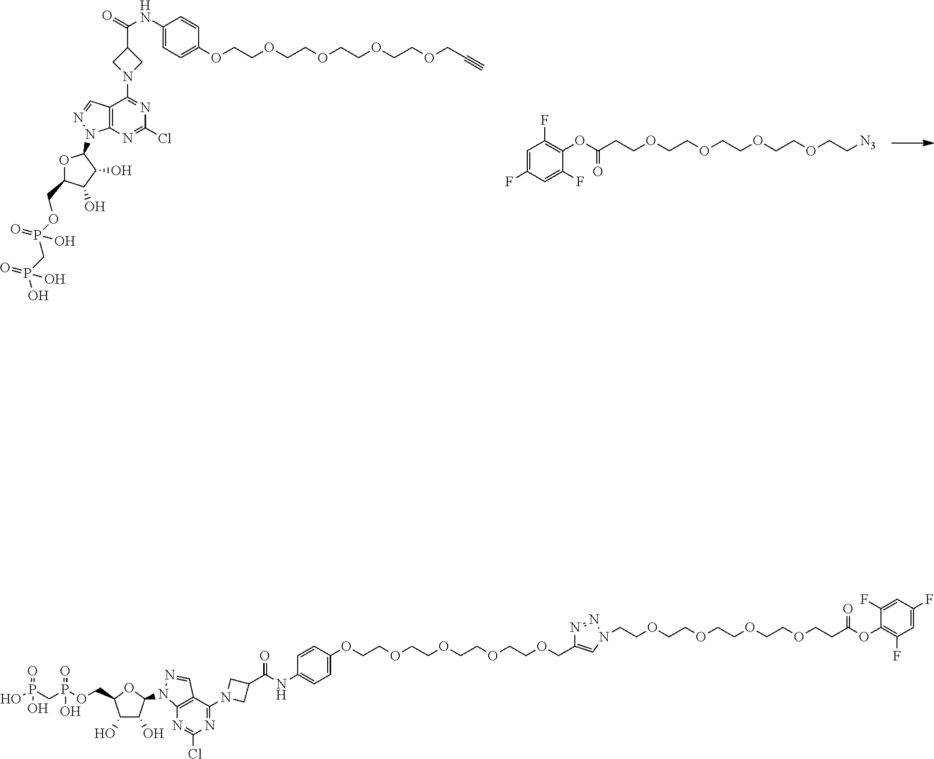 [SMILES: C#CCOCCOCCOCCOCCOc1ccc(NC(=O)C2CN(c3nc(Cl)nc4c3cnn4[C@@H]3O[C@H](COP(=O)(O)CP(=O)(O)O)[C@@H](O)[C@H]3O)C2)cc1 ; [N-]=[N+]=NCCOCCOCCOCCOCCC(=O)Oc1c(F)cc(F)cc1F ; O=C(CCOCCOCCOCCOCCn1cc(COCCOCCOCCOCCOc2ccc(NC(=O)C3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F]

### Step a

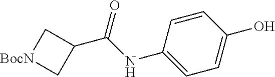 [SMILES: CC(C)(C)OC(=O)N1CC(C(=O)Nc2ccc(O)cc2)C1]

1-[(Tert-butyl)oxycarbonyl]azetidine-3-carboxylic acid (500 mg, 2.48 mmol), HATU (1.42 g, 3.73 mmol) and DIEA in DMF (0.19 mL, 2.48 mmol) were stirred together for 10 minutes at ambient temperature. To this mixture 4-hydroxyaniline (406 mg, 3.72 mmol) in DMF (1 mL) was added and the resulting mixture was stirred for 1 hour at ambient temperature. The reaction was concentrated under reduced pressure and purified by reversed phase HPLC (5% to 100% ACN/water). The product was a white solid. Yield 540 mg, 74.3%. Ions found by LCMS: [M-C(CH₃)₃+H]⁺=237.2.

### Step b

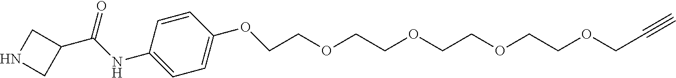 [SMILES: C#CCOCCOCCOCCOCCOc1ccc(NC(=O)C2CNC2)cc1]

To a solution of the compound from previous step (540 mg, 1.85 mmol) in acetonitrile (30 mL) was added potassium carbonate (510 mg, 3.69 mmol) and propargyl-PEG₄-mesylate (867 mg, 2.21 mmol). The resulting mixture was heated at reflux for 16 hours. The mixture was cooled and the excess solvent was removed under reduced pressure. Ethyl acetate (50 mL) was added to the crude material and washed with water and brine. Organic layer was dried over sodium sulfate, filtered and concentrated under reduced pressure to obtain crude N-Boc protected derivative of the title compound which was purified by reversed phase HPLC. Light yellow viscous liquid. Yield 710 mg, 76%. Ions found by LCMS: [M-Boc+H]⁺=407.2. To a solution of the N-Boc protected compound (710 mg, 1.40 mmol) in dioxane 10 mL was added 4M HCl solution in dioxane (7 mL, 28 mmol). The resulting mixture was stirred at ambient temperature for 2 hours, concentrated and dried under high vacuum to afford the amine intermediate as an HCl salt. Light yellow solid. Yield 602 mg, 97%. Ions found by LCMS: [M+H]⁺=407.2.

### Step c

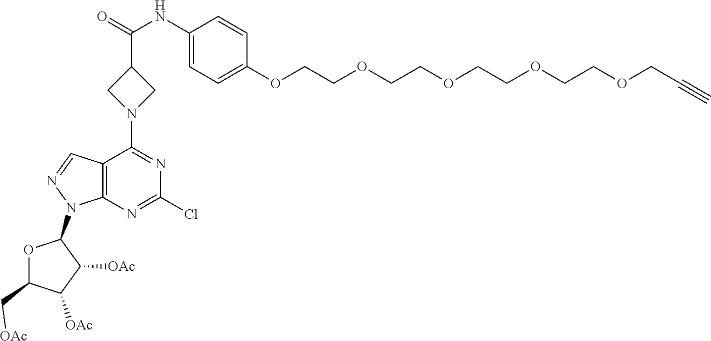 [SMILES: C#CCOCCOCCOCCOCCOc1ccc(NC(=O)C2CN(c3nc(Cl)nc4c3cnn4[C@@H]3O[C@H](COC(C)=O)[C@@H](OC(C)=O)[C@H]3OC(C)=O)C2)cc1]

A mixture of intermediate B (250 mg, 0.56 mmol), the amine from the previous step (296m g, 0.67 mmol), and triethylamine (0.16 mL, 1.12 mmol) in ethanol (6 mL, 1.12 mmol) were heated at 50° C. for 1 hour. The mixture was cooled to ambient temperature and concentrated under reduced pressure. The crude residue was redissolved in ethyl acetate (50 mL) and washed with water and brine. The combined organic layers were dried over sodium sulfate, filtered and concentrated to afford the product as a light yellow solid which was used for the next step without further purification. Yield 437 mg, 96%. Ions found by LCMS: [M+H]⁺=817.2.

### Step d

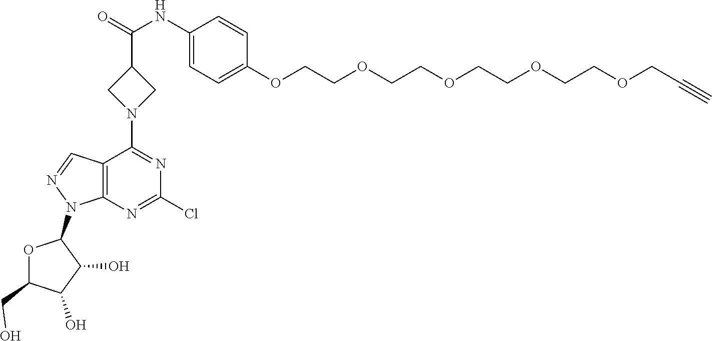 [SMILES: C#CCOCCOCCOCCOCCOc1ccc(NC(=O)C2CN(c3nc(Cl)nc4c3cnn4[C@@H]3O[C@H](CO)[C@@H](O)[C@H]3O)C2)cc1]

The product from the previous step (0.437 g, 0.535 mmol) in methanol (5 mL) was treated with potassium carbonate (0.258 g, 1.87 mmol) at ambient temperature for 2 hours. The mixture was concentrated under reduced pressure, dissolved in minimum 1:1 MeOH:H₂O and then purified by reversed phase HPLC (10% to 100% ACN/water, 0.1% TFA). The product was a white solid. Yield 0.330 g, 89%. Ions found by LCMS: [M+H]⁺=691.2.

### Step e

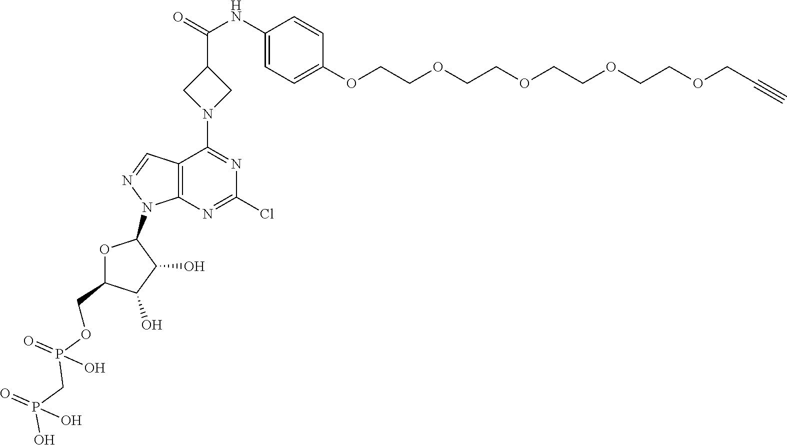

To a solution of the product from previous step (50 mg, 0.07 mmol) and 2,2-dimethoxypropane (0.044 mL, 0.36 mmol) in acetone (3 mL) at ambient temperature was added p-TsOH (2 mg, 0.007 mmol). The reaction was stirred for two hours then concentrated under reduced pressure. The crude residue was dissolved in ethyl acetate (10 mL) and washed with saturated sodium bicarbonate. The organic layer was collected and dried over anhydrous Na₂SO₄, filtered and concentrated in vacuo to provide the acetonide derivative. The product was a white solid. LCMS[M+H]⁺=731.2. To a suspension of methylenebis(phosphonic dichloride) (54 mg, 0.22 mmol) in THE (2 mL) at 0° C. was added DIEA (0.014 mL, 0.08 mmol). To the resulting mixture was added to a solution of acetonide derivative in THE (2 mL) dropwise over the course of 5 minutes. Following addition, the resulting mixture was stirred at 0° C. for an additional 15 minutes, then the solution was transferred to a pre-cooled (0° C.) flask containing 0.2 M aqueous HCl. The reaction mixture was warmed to ambient temperature and stirred for additional 4 hours. Upon completion (by LCMS), the reaction mixture was concentrated and purified by reversed phase HPLC using ACN: water (0.1% TFA modifier). The product was a white solid. Yield 37 mg, 60%. Ions found by LCMS: [M+H]⁺=849.2.

### Step f

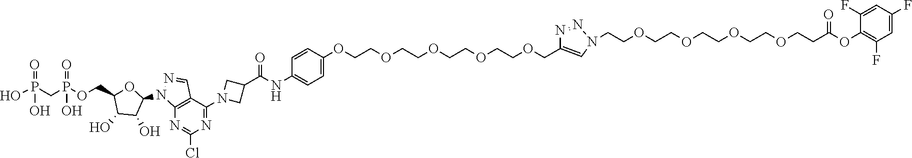 [SMILES: O=C(CCOCCOCCOCCOCCn1cc(COCCOCCOCCOCCOc2ccc(NC(=O)C3CN(c4nc(Cl)nc5c4cnn5[C@@H]4O[C@H](COP(=O)(O)CP(=O)(O)O)[C@@H](O)[C@H]4O)C3)cc2)nn1)Oc1c(F)cc(F)cc1F]

A solution of product from the previous step (0.03 g, 0.035 mmol), and intermediate A (0.015 g, 0.035 mmol) dissolved in DMF:H₂O (1:3, 1.5 mL) was cooled to 0° C. To this a pre-mixed solution of a solution of copper(II) sulfate (0.0006 g, 0.0035 mmol), sodium ascorbate (0.021 g, 0.106 mmol), and BTTA (0.003 g, 0.007 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). The product was a white solid. Yield 50 mg, 54%. Ions found by LCMS: [(M+2H)/2]⁺=635.8.

### Synthesis of Conjugate 62

Trifluorophenol ester (19 mg, 0.01448 mmol) described in the synthesis of Int-117) was conjugated to Fc carrier SEQ ID NO: 13 (100 mg, 5.15 mL in PBS at pH 7.4, 0.0018 mmol) as described in the conjugation procedure for Conjugate 60. Maldi TOF analysis of the purified final product gave an average mass of 62,537 Da (DAR 3.8). Yield 66.9 mg, 66.9%.

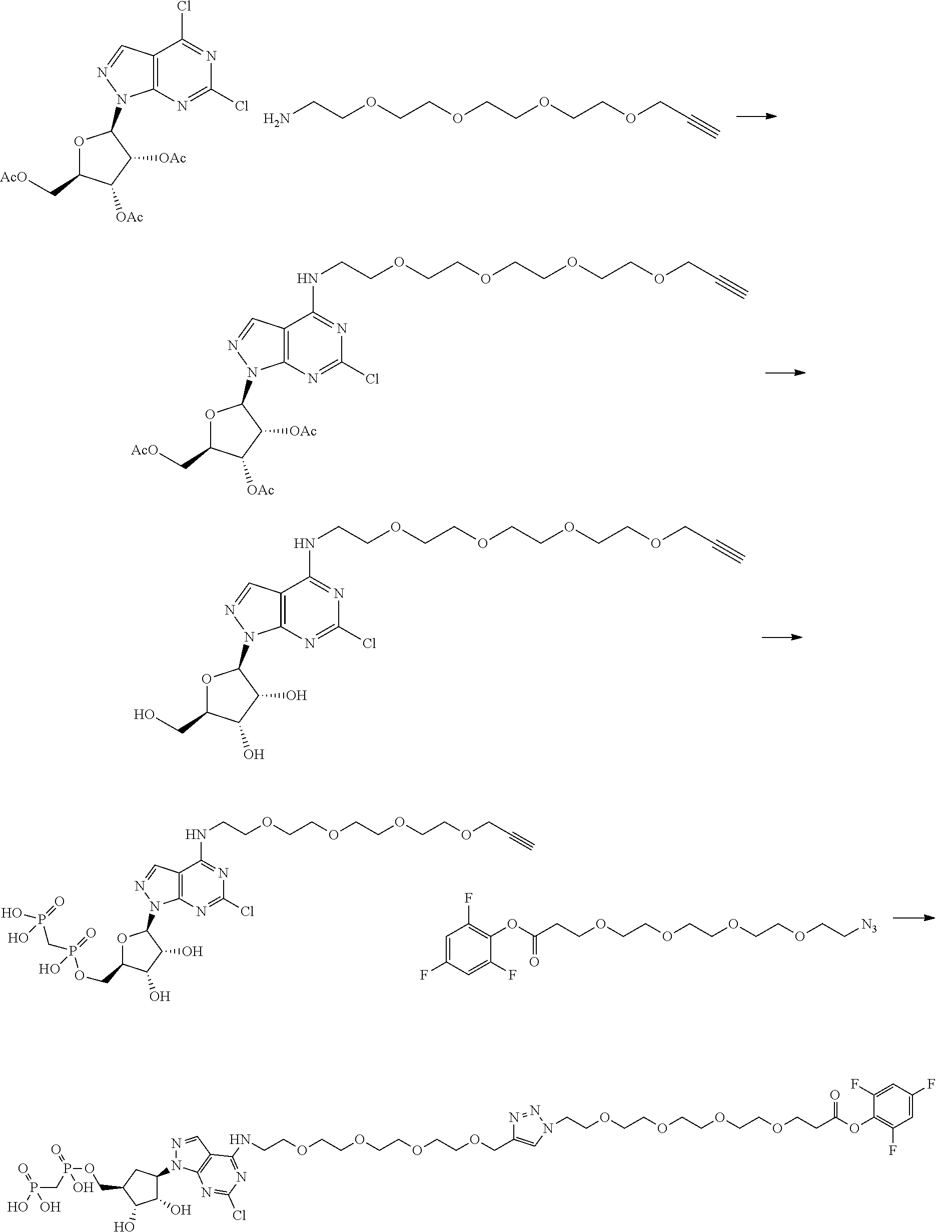

### Synthesis of Int-119

### Step a

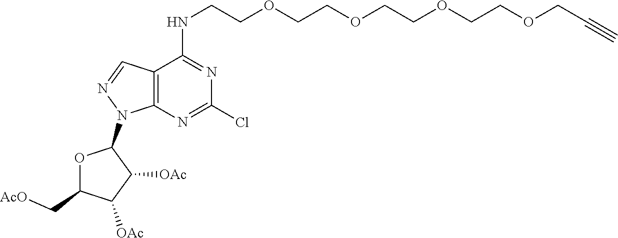 [SMILES: C#CCOCCOCCOCCOCCNc1nc(Cl)nc2c1cnn2[C@@H]1O[C@H](COC(C)=O)[C@@H](OC(C)=O)[C@H]1OC(C)=O]

A mixture of intermediate B (250 mg, 0.56 mmol), propargyl-PEG₄-amine (155 mg, 0.67 mmol) and triethylamine (0.16 mL, 1.12 mmol) in ethanol (6 mL) were heated at 50° C. for 1 hour. The mixture was cooled to ambient temperature and concentrated under reduced pressure. The crude residue was redissolved in ethyl acetate (50 mL) and washed with water and brine. The organic layer was dried over sodium sulfate and solvent was removed under reduced pressure to yield the product as a colorless viscous oil, which was used for the next step without further purifications Yield 340 mg, 95%. Ions found by LCMS: [M+H]⁺=642.2.

### Step b

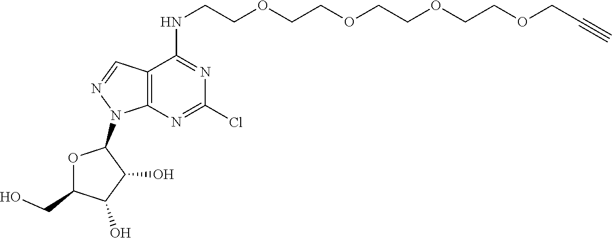 [SMILES: C#CCOCCOCCOCCOCCNc1nc(Cl)nc2c1cnn2[C@@H]1O[C@H](CO)[C@@H](O)[C@H]1O]

The product from the previous step (340 mg, 0.53 mmol) in methanol (5 mL) was treated with potassium carbonate (255 mg, 1.85 mmol) at ambient temperature for 2 hours. The mixture was concentrated under reduced pressure and purified by silica gel column chromatography using DCM to 10% MeOH in DCM. The product was a white solid. Yield: 240 mg, 88%. Ions found by LCMS: [M+H]⁺=516.2.

### Step c

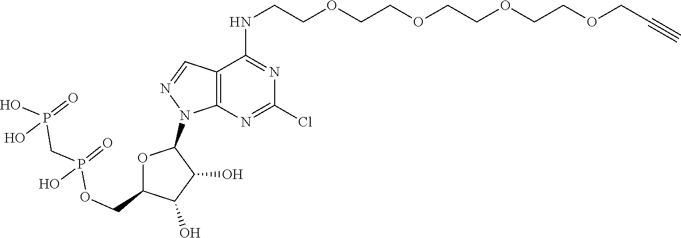 [SMILES: C#CCOCCOCCOCCOCCNc1nc(Cl)nc2c1cnn2[C@@H]1O[C@H](COP(=O)(O)CP(=O)(O)O)[C@@H](O)[C@H]1O]

To a solution of the product from previous step (240 mg, 0.47 mmol) and 2,2-dimethoxypropane (0.28 mL, 2.33 mmol) in acetone (4 mL) at room temperature was added p-TsOH (8 mg, 0.046 mmol). The reaction was stirred for 2 hours then concentrated under reduced pressure. The crude residue was re-dissolved in ethyl acetate (20 mL) and washed with saturated sodium bicarbonate. The organic layer was collected and dried over sodium sulfate, filtered and concentrated under reduced pressure to provide the acetonide derivative. The product was a white solid. LCMS[M+H]⁺=556.2. To a suspension of methylenebis(phosphonic dichloride) (348 mg, 1.4 mmol) in THE (5 mL) at 0° C. was added DIEA (0.089 mL, 0.51 mmol). To the resulting mixture was added a solution of the acetonide derivative in THE (2 mL) dropwise over the course of 5 minutes. Following addition, the resulting mixture was stirred at 0° C. for an additional 15 minutes, then the solution was transferred to a pre-cooled (0° C.) flask containing 0.2 M aqueous HCl (3 mL). The reaction mixture was warmed to ambient temperature and stirred for an additional 4 hours. Upon completion (by LCMS), the reaction mixture was concentrated and purified by reversed HPLC using ACN: water (0.1% TFA modifier). The product was a white solid. Yield 100 mg, 32%. Ions found by LCMS: [M+H]⁺=674.2.

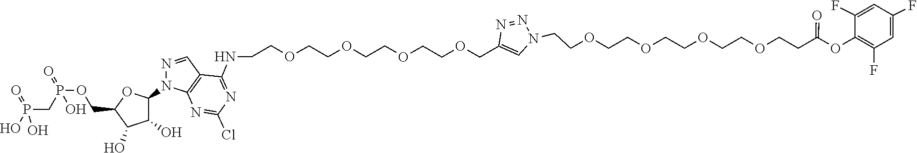 [SMILES: O=C(CCOCCOCCOCCOCCn1cc(COCCOCCOCCOCCNc2nc(Cl)nc3c2cnn3[C@@H]2O[C@H](COP(=O)(O)CP(=O)(O)O)[C@@H](O)[C@H]2O)nn1)Oc1c(F)cc(F)cc1F]

Product from previous step (0.06 g, 0.076 mmol), and intermediate A (0.032 g, 0.076 mmol) dissolved in DMF:H₂O (1:3, 1.5 mL) was cooled to 0° C. To this a pre-mixed solution of a solution of copper(II) sulfate (0.0012 g, 0.0076 mmol), sodium ascorbate (0.045 g, 0.23 mmol), and BTTA (0.007 g, 0.015 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA). The product was a white solid. Yield 104 mg, 53.5%. Ions found by LCMS: [(M+2H)/2]⁺=548.2.

### Synthesis of Conjugate 63a

Trifluorophenol ester (16 mg, 0.01448 mmol) described in the synthesis of Int-119 was conjugated to Fc carrier SEQ ID NO: 13 (100 mg, 3.47 mL in PBS at pH 7.4 as, 0.0018 mmol) described in the conjugation procedure for Conjugate 61. Maldi TOF analysis of the purified final product gave an average mass of 62259 Da (DAR=3.8). Yield 59.2 mg, 59.2%.

### Synthesis of Int-130

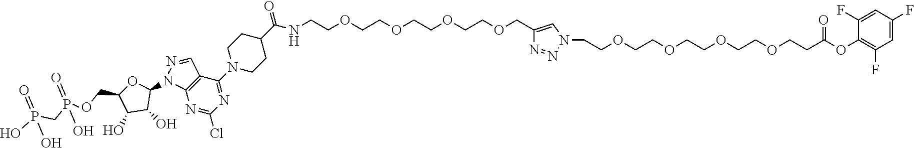 [SMILES: O=C(CCOCCOCCOCCOCCn1cc(COCCOCCOCCOCCNC(=O)C2CCN(c3nc(Cl)nc4c3cnn4[C@@H]3O[C@H](COP(=O)(O)CP(=O)(O)O)[C@@H](O)[C@H]3O)CC2)nn1)Oc1c(F)cc(F)cc1F]

The title compound was prepared analogously to Int-10 where N-Boc azetidine-3-carboxylic acid was replaced with N-Boc-piperidine-4-carboxylic acid. The product was a white solid. Yield 72 mg, 78.1%. Ions found by LCMS: [(M+2H)/2]⁺=548.2.

### Synthesis of Conjugate 69

Trifluorophenol ester (18 mg, 0.01448 mmol) described in the synthesis of Int-130 was conjugated to Fc carrier SEQ ID NO: 13 (100 mg, 5.15 mL in PBS at pH 7.4, 0.0018 mmol) described in the conjugation procedure for Conjugate 61. MALDI TOF analysis of the purified final product gave an average mass of 62,259 Da (DAR 3.8). Yield 59.2 mg, 59.2%.

### Synthesis of Int-134

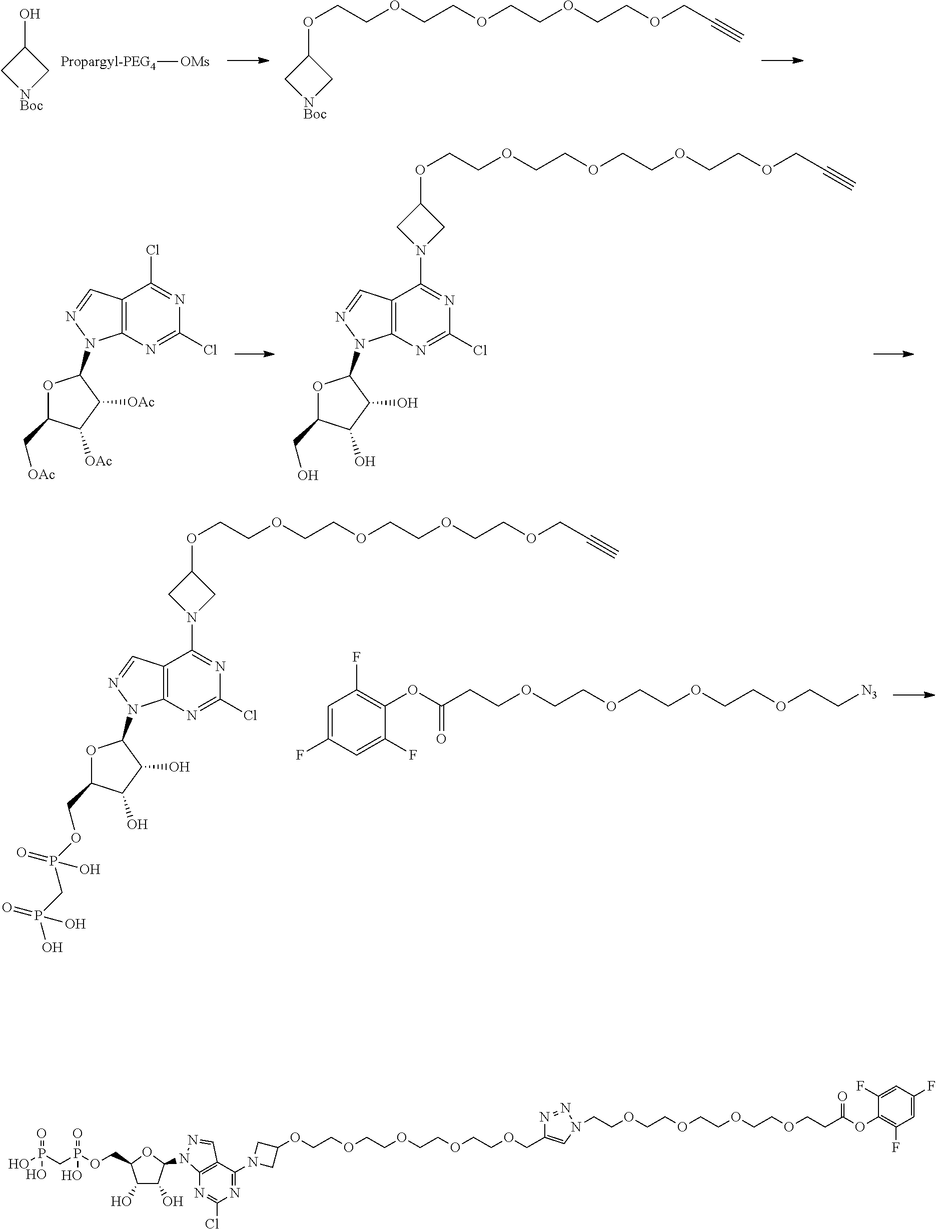

### Step a

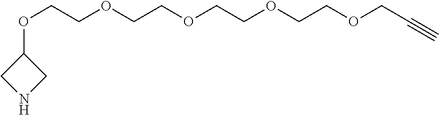 [SMILES: C#CCOCCOCCOCCOCCOC1CNC1]

To a solution of N-Boc-3-hydroxyazetidine (346 mg, 2 mmol) in DMF (10 mL) at 0° C. was added sodium hydride (60% in mineral oil, 119 mg, 3 mmol) in portions, and the mixture was stirred for 1 hour at ambient temperature. Propargyl-PEG₄-mesylate (620 mg, 2 mmol) was added to the sodium alkoxide suspension and the reaction mixture was stirred for 16 hours at ambient temperature. The resulting solution was quenched by saturated ammonium chloride solution (20 mL), extracted with ethyl acetate (3×20 mL) then washed with water and brine. The combined organic extracts were dried over sodium sulfate, filtered and concentrated. The residue was purified by column chromatography (Hexanes: EtOAc) to yield the N-Boc protected intermediate as a colorless viscous oil. Ions found by LCMS: [M+H]⁺=388.2. The N-Boc protected intermediate (0.640 g) was dissolved in 4N HCl in dioxane (10 mL) and stirred for 4 hours at ambient temperature. The solvent was removed under reduced pressure and dried under high vacuum to afford the compound as a viscous liquid. Yield 530 mg, 83%, 2 steps. Ions found by LCMS: [M+H]⁺=288.2.

### Step b

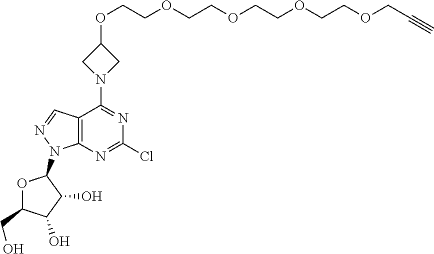

A mixture of intermediate B (250 mg, 0.56 mmol), the amine hydrochloride salt from the previous step (217 mg, 0.68 mmol), and triethylamine (0.16 mL) in methanol (6 mL) were heated at 50° C. for 2 hours. After complete consumption of the starting materials (by LCMS), the reaction mixture was cooled to ambient temperature and the mixture was concentrated under reduced pressure. The crude residue was dissolved in ethyl acetate (50 mL) and washed with water and brine. The combined organic extracts were dried over sodium sulfate. The solvent was removed under reduced pressure to yield the triacetate derivative as a yellow viscous oil which was used for the next step without further purifications. Ions found by LCMS[M+H]⁺=668.2. The triacetate intermediate (370 mg, 0.56 mmol) was dissolved in methanol (6 mL), potassium carbonate (270 mg, 1.95 mmol) was added, and the reaction was stirred at ambient temperature for 2 hours. The mixture was concentrated under reduced pressure to afford the crude product. The crude material was dissolved in minimum 1:1 MeOH: H₂O and purified by reversed phase HPLC (5% to 100% ACN/water, 0.1% TFA modifier) to yield the compound. The product was a white foam. Yield 280 mg, 88%. Ions found by LCMS: [M+H]⁺=572.2.

### Step c

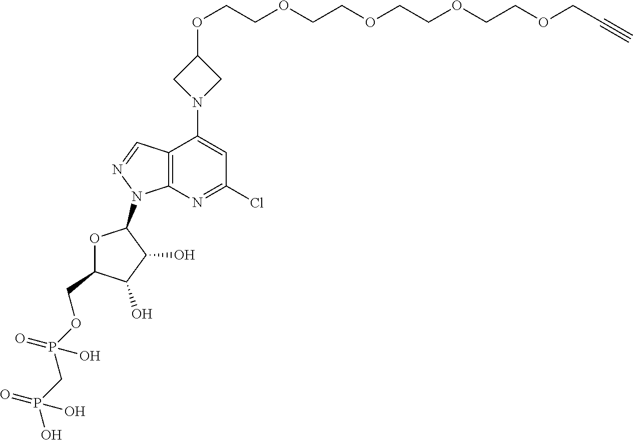

To a solution of the triol product from the previous step (280 mg, 0.49 mmol) and 2,2-dimethoxypropane (0.3 mL, 2.45 mmol) in acetone (4 mL) at ambient temperature was added p-TsOH (8 mg, 0.0.048 mmol). The reaction was stirred for 2 hours then concentrated under reduced pressure. The crude residue was dissolved in ethyl acetate (20 mL) and washed with saturated sodium bicarbonate. The organic layer was collected and dried over sodium sulfate, filtered and concentrated to afford the acetonide derivative an off-The product was a white solid. Ions found by LCMS: [M+H]⁺=612.2. To a suspension of methylenebis(phosphonic dichloride) (367 mg, 1.4 mmol) in THE (5 mL) at 0° C. was added DIEA (0.093 mL, 0.54 mmol). To the resulting mixture was added a solution of the acetonide derivative in THE (2 mL) dropwise over the course of 5 minutes. Following addition, the resulting mixture was stirred at 0° C. for an additional 15 minutes, then the solution was transferred to a pre-cooled (0° C.) flask containing 0.2 N aqueous HCl. The reaction mixture was warmed to ambient temperature and stirred for an additional 4 hours. Upon completion (by LCMS), the reaction mixture was concentrated and purified by reversed phase HPLC using ACN: water (0.1% TFA modifier). The product was a white solid. Yield 280 mg, 78%. Ions found by LCMS: 730.2[M+H]⁺.

### Step d

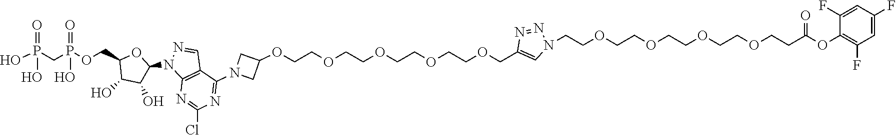 [SMILES: O=C(CCOCCOCCOCCOCCn1cc(COCCOCCOCCOCCOC2CN(c3nc(Cl)nc4c3cnn4[C@@H]3O[C@H](COP(=O)(O)CP(=O)(O)O)[C@@H](O)[C@H]3O)C2)nn1)Oc1c(F)cc(F)cc1F]

A solution of product from the previous step (0.06 g, 0.082 mmol), and intermediate A (0.035 g, 0.082 mmol) dissolved in DMF:H₂O (1:3, 1.5 mL) was cooled to 0° C. To this a pre-mixed solution of a solution of copper(II) sulfate (0.002 g, 0.0082 mmol), sodium ascorbate (0.048 g, 0.246 mmol), and BTTA (0.007 g, 0.016 mmol) dissolved in water (0.5 mL) was added and stirred for 5 min at the same temperature and gradually warmed to room temperature and stirred at room temperature for 15 min. After completion of the reaction, the reaction mixture was quenched by the addition of few drops of AcOH and EDTA to pH of 6 and the product was purified by reverse phase HPLC (10% to 100% ACN/water, 0.1% TFA modifier). The product was a white solid. Yield 52 mg, 55%. Ions found by LCMS: [(M+2H)/2]⁺=576.2.

### Synthesis of Conjugate 71

Trifluorophenol ester (17 mg, 0.0144 mmol) (described in the synthesis of Int-134) in DMF (0.5 mL) was added to Fc carrier SEQ ID NO: 13 (100 mg in 5.15 mL PBS at pH 7.4) then adjusted to pH-7.4 with sodium carbonate buffer. The mixture was agitated at ambient temperature for 4 hours. The reaction was quenched by stirring in a 150 mM His/100 mM ammonium hydroxide buffer (pH 8.5) for 12 hours and purified by dialyze into in 150 mM His pH′8.5 buffer, protein A and SEC column. Maldi TOF analysis of the purified final product gave an average mass of 62,647 Da. (DAR of 4.4). Yield 43.7 mg, 43.7%.

### Synthesis of Int-136

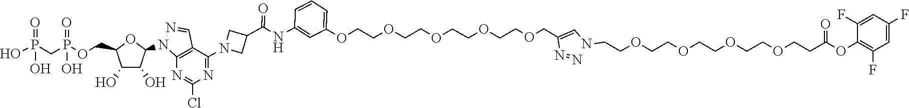 [SMILES: O=C(CCOCCOCCOCCOCCn1cc(COCCOCCOCCOCCOc2cccc(NC(=O)C3CN(c4nc(Cl)nc5c4cnn5[C@@H]4O[C@H](COP(=O)(O)CP(=O)(O)O)[C@@H](O)[C@H]4O)C3)c2)nn1)Oc1c(F)cc(F)cc1F]

The title compound was prepared analogously to step e of Int-117 where 4-hydroxyaniline was replaced with 3-hydroxyaniline. The product was a white solid. Yield 32.8 mg, 73.1%. Ions found by LCMS: [(M+2H)/2]⁺=635.8.

### Synthesis of Conjugate 72

Trifluorophenol ester (17 mg, 0.01448 mmol) described in the synthesis of Int-136 was conjugated to Fc carrier SEQ ID NO: 13 (100 mg, 3.4 mL in PBS at pH 7.4 as, 0.0018 mmol) described in the conjugation procedure for Conjugate 71. Maldi TOF analysis of the purified final product gave an average mass of 64,461 Da (DAR=5.6). Yield 47.9 mg, 47.9%.

### Synthesis of Int-2

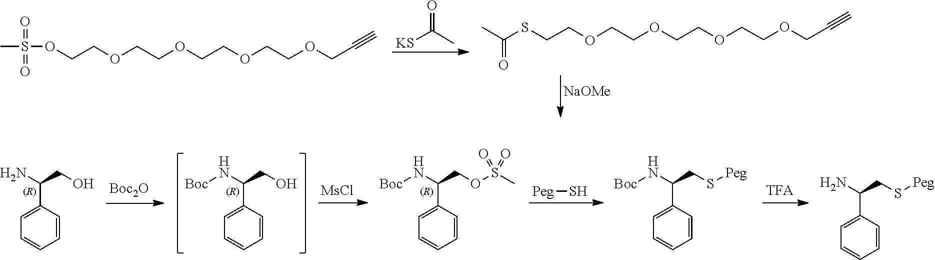

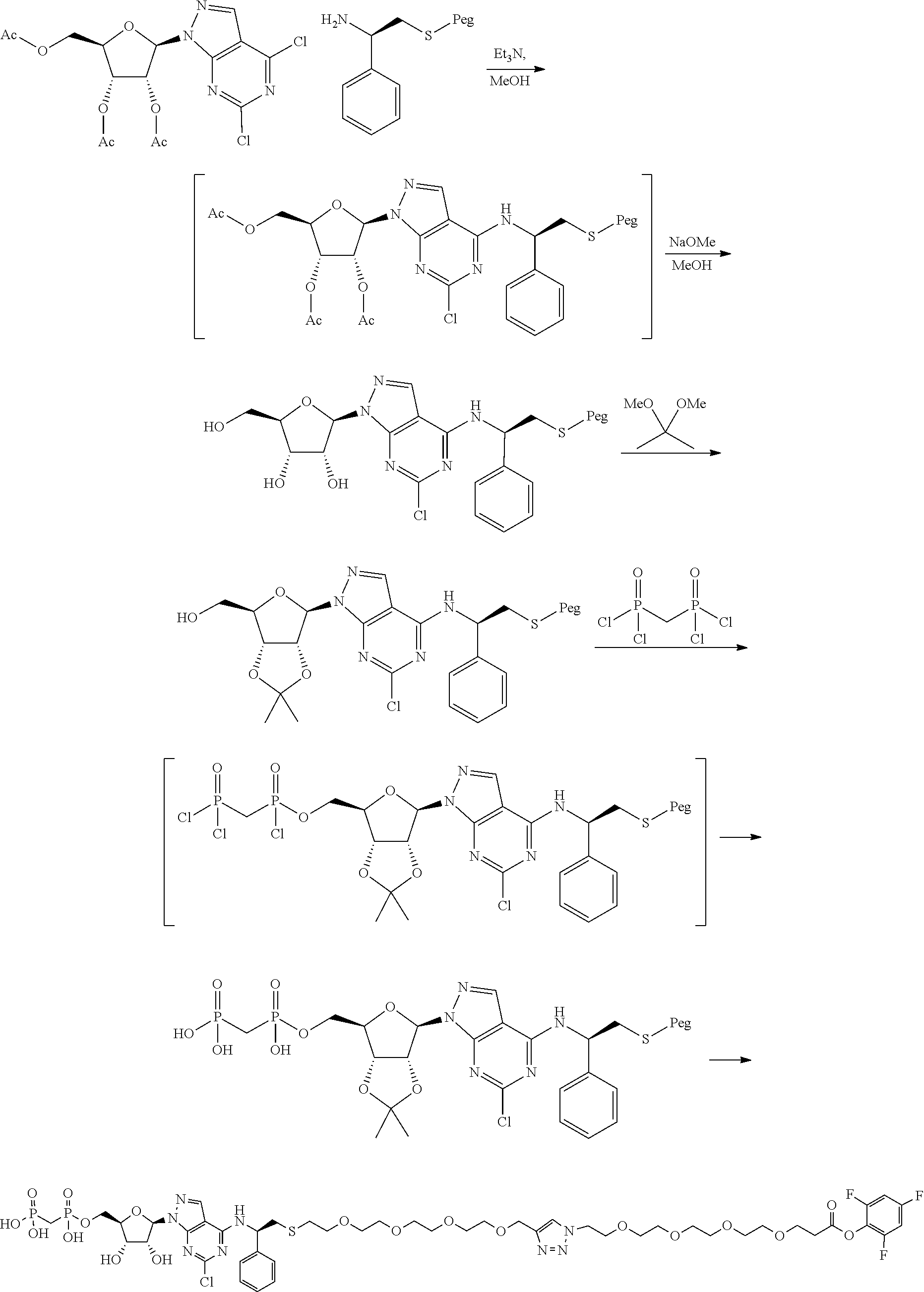

### Step a

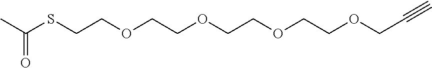 [SMILES: C#CCOCCOCCOCCOCCSC(C)=O]

A solution of propargyl-Peg4-mesylate (1.00 g, 3.22 mmol) and potassium thioacetate (0.74 g, 6.44 mmol) were dissolved in DMF (12 mL), then vacuum flushed with nitrogen. After stirring for 5 min the reaction became viscous, so more DMF (6 mL) was added. The reaction was stirred at room temperature overnight, at which point the color was dark red. Work-up: the crude reaction was diluted with water and extracted with DCM several times. The DCM extracts were dried with sodium sulfate, filtered, concentrated, and purified by RPLC (10% acetonitrile/water to 100% acetonitrile). Yield 927 mg, 99%. Ions found by LCMS: [M+H]⁺=291.2

### Step b

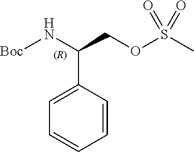

A solution of (R)-phenylglycinol (1.372 g, 10.0 mmol) dissolved in THE (20 mL), was treated with Boc anhydride (2.40 g, 11.0 mmol) at room temperature. After 15 min LCMS showed the Boc protection was complete. The reaction was then treated with DIEA (2.09 mL, 12.0 mmol), and methanesulfonyl chloride (0.85 mL, 11.0 mmol), and stirred for 1 h, at which time LCMS showed complete conversion to mesylate. The crude reaction was diluted with ethyl acetate, extracted with water, 1 N HCl, brine, and dried over sodium sulfate and concentrated. Upon concentrating crystals formed. The filtrate was treated with hexanes to improve recovery of product. The product was a crystalline white solid, yield 2.21 g, 70%. Ions found by LCMS: [M −Boc+H]⁺=216.2

### Step c

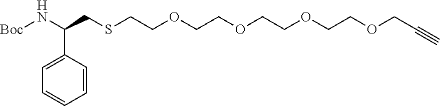 [SMILES: C#CCOCCOCCOCCOCCSC[C@H](NC(=O)OC(C)(C)C)c1ccccc1]

Peg-thioacetate (0.45 g, 1.55 mmol) from step a of this example, was dissolved in DMF (4.5 mL), and vacuum flushed several times, then treated with sodium methoxide (0.39 mL, 1.70 mmol, 25% solution in methanol). After stirring for 20 min at room temperature, mesylate (0.489 g, 1.55 mmol) from the previous step was added in one portion. The reaction was further stirred for 30 min, at which time LCMS showed complete conversion. The reaction was made slightly acidic with acetic acid, concentrated to an oil, and purified by RPLC (10% acetonitrile/water to 100% acetonitrile). Yield 435 mg, 60%. Ions found by LCMS: [M+Na]⁺=490.2

### Step d

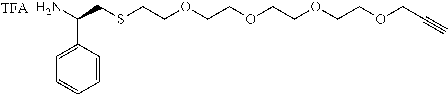 [SMILES: C#CCOCCOCCOCCOCCSC[C@H](N)c1ccccc1]

Product from the previous step (435 mg, 0.93 mmol) was treated with a 1:1 solution of DCM and TFA (4 mL), then stirred at room temperature for 30 minutes. The crude reaction was concentrated to an oil and used in the next step without further purification.

### Step e

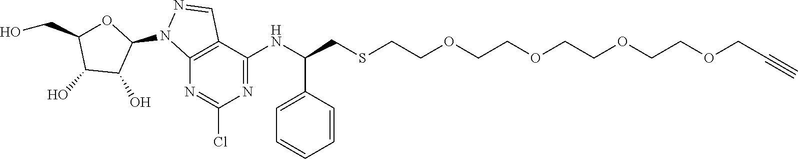 [SMILES: C#CCOCCOCCOCCOCCSC[C@H](Nc1nc(Cl)nc2c1cnn2[C@@H]1O[C@H](CO)[C@@H](O)[C@H]1O)c1ccccc1]

Crude product from the previous step (342 mg, 0.93 mmol), intermediate B (416 mg, 0.93 mmol), and DIEA (0.648 mL, 3.72 mmol), were mixed in methanol (5 mL) at room temperature. LCMS after 5 minutes shows ˜90% conversion to product. LCMS after 2 hours showed complete consumption of starting materials. Sodium methoxide (0.851 mL, 3.72 mmol, 25% sodium methoxide in methanol) was added at room temperature. LCMS after 10 min showed complete deprotection of acetates. Reaction was neutralized with acetic acid, concentrated, and purified by RPLC (10% acetonitrile/water to 100% acetonitrile with 0.1% TFA). Yield 432 mg, 71%. Ions found by LCMS: [M+H]⁺=652.2

### Step f

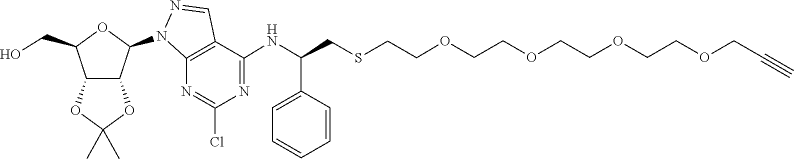 [SMILES: C#CCOCCOCCOCCOCCSC[C@H](Nc1nc(Cl)nc2c1cnn2[C@@H]1O[C@H](CO)[C@H]2OC(C)(C)O[C@H]21)c1ccccc1]

Product from the previous step (432 mg, 0.662 mmol), dissolved in acetone (3 mL), was treated with dimethoxy propane (3 mL) and toluene sulfonic acid (12.6 mg, 0.066 mmol). LCMS after 1 hour showed complete conversion to the acetonide. Work-up: the reaction was diluted with ethyl acetate, washed with aqueous, saturated sodium bicarbonate, dried over sodium sulfate, concentrated, and purified by flash chromatography (10% EtOAc/DCM to 100% EtOAc). Yield 214 mg, 47%. Ions found by LCMS: [M+H]⁺=692.2

### Step g

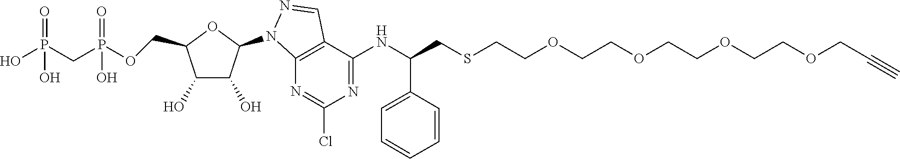 [SMILES: C#CCOCCOCCOCCOCCSC[C@H](Nc1nc(Cl)nc2c1cnn2[C@@H]1O[C@H](COP(=O)(O)CP(=O)(O)O)[C@@H](O)[C@H]1O)c1ccccc1]

Product from the previous step (214 mg, 0.309 mmol), dissolved in trimethoxyphosphate (6 mL), was treated with methylene (bis phosphonic dichloride) (386 mg, 1.55 mmol) at ambient temperature. LCMS shows that all starting material had been consumed after 2 hours. The reaction was cooled with an ice bath then treated with 2M HCl (6 mL) and stirred at room temperature. LCMS after 2 hours showed complete acetonide deprotection. The reaction was concentrated by rotary evaporation, then purified by RPLC (10% acetonitrile/water to 100% acetonitrile with 0.1% TFA). Yield 160 mg, 64%. Ions found by LCMS: [M+H]⁺=810.2

### Step h

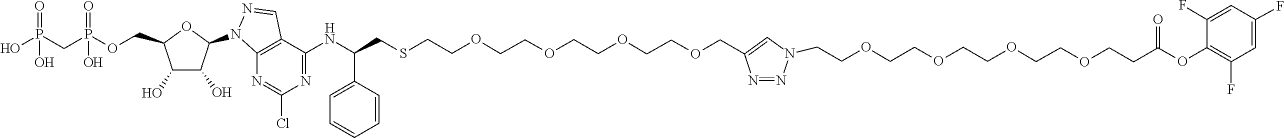 [SMILES: O=C(CCOCCOCCOCCOCCn1cc(COCCOCCOCCOCCSC[C@H](Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F]

To a solution of product from the previous step (50 mg, 0.062 mmol) and azido intermediate A (34 mg, 0.080 mmol), dissolved in DMF (1 mL), was added a solution of copper(II)sulfate (2.5 mg, 0.0154 mmol), sodium ascorbate (12.2 mg, 0.062 mmol), and THPTA (10.7 mg, 0.025 mmol) dissolved in water (0.5 mL). After stirring at room temperature for 20 minutes, LCMS shows complete conversion to product. The reaction was purified directly by RPLC (10% acetonitrile/water to 100% acetonitrile with 0.1% TFA). Yield 49 mg, 64%. Ions found by LCMS: [(M+2H)/2]⁺=616.2

### Synthesis of Conjugate 2

A solution of trifluorophenyl ester (10.6 mg, 0.0086 mmol, Int-2), dissolved in DMF (1 mL), was added to a solution of Fc (50 mg, 0.00086 mmol, SEQ ID NO: 17 in PBS at pH 7.4, 17.3 mg/mL) at ambient temperature. The pH of the resulting solution was adjusted to ˜8.5 with borate buffer (300 uL, 1M, pH 8.5). The homogeneous colorless reaction was rocked gently for 3 hours and then submitted for purification according to the general procedure. Maldi TOF analysis of the purified final product gave an average mass of 62,957 Da (DAR=4.3). Yield: 41 mg, 83%.

### Synthesis of Conjugate 146

The title compound was prepared analogously to Conjugate 2, where Fc SEQ ID NO: 17 was replaced with Fc SEQ ID NO: 13.

### Synthesis of Int-1

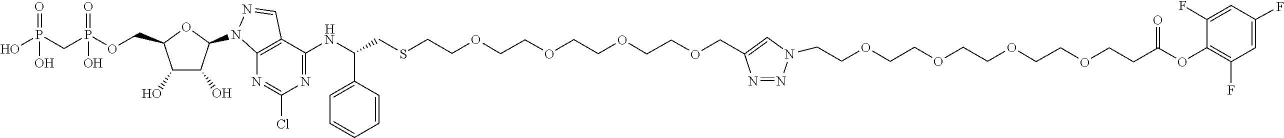 [SMILES: O=C(CCOCCOCCOCCOCCn1cc(COCCOCCOCCOCCSC[C@@H](Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F]

The title compound was prepared analogously to Int-2, where (R)-phenylglycinol was replaced with (S)-phenylglycinol in step a of the Example.

### Synthesis of Conjugate 1

The title conjugate was prepared analogously to Conjugate 2, where the trifluorophenyl ester from Int-2 was replaced with the trifluorophenyl ester from Int-1.

### Synthesis of Conjugate 144

The title compound was prepared analogously to Conjugate 1, where Fc SEQ ID NO: 17 was replaced with Fc SEQ ID NO: 13.

### Synthesis of Int-14

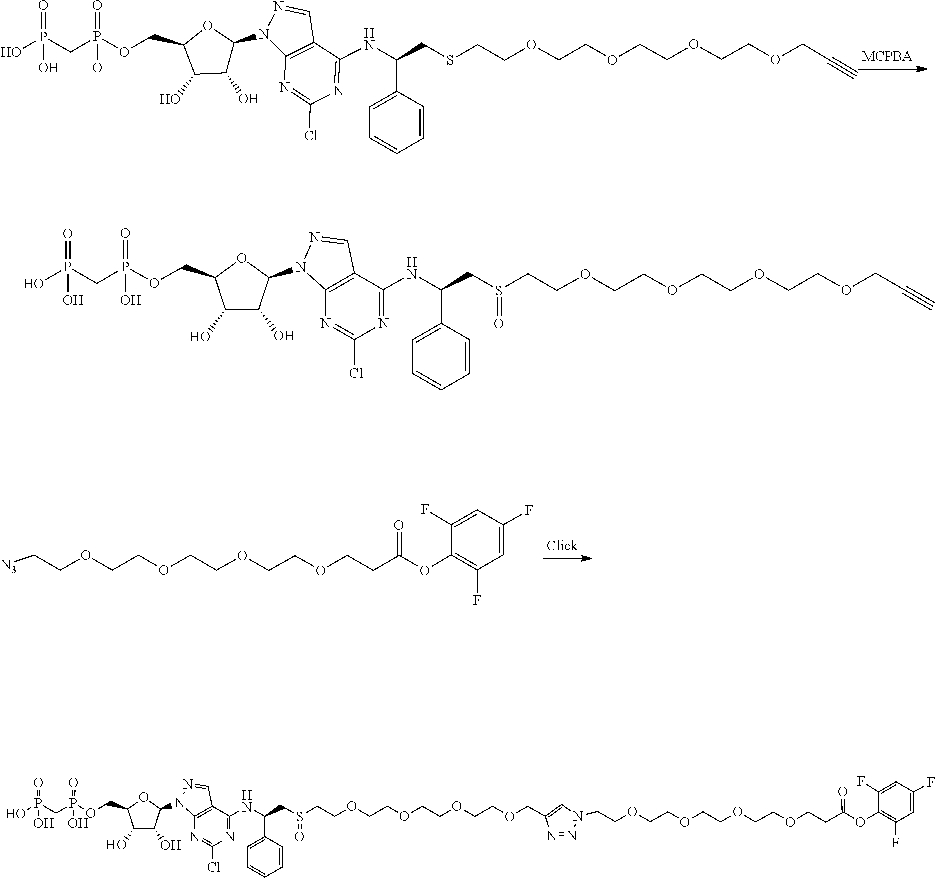

### Step a

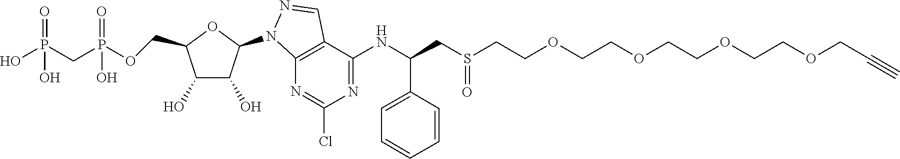 [SMILES: C#CCOCCOCCOCCOCCS(=O)C[C@H](Nc1nc(Cl)nc2c1cnn2[C@@H]1O[C@H](COP(=O)(O)CP(=O)(O)O)[C@@H](O)[C@H]1O)c1ccccc1]

A solution of thioether (56 mg, 0.069 mmol, described in Int-2), dissolved in THE (2 mL), was treated with MCPBA (12.5 mg, 0.073 mmol). LCMS after 5 minutes, shows clean conversion to sulfoxide. The product was purified directly by RPLC (5% acetonitrile/water to 100% acetonitrile with 0.1% TFA). Yield 55 mg, 96%. Ions found by LCMS: [M+H]⁺=826.2.

### Step b

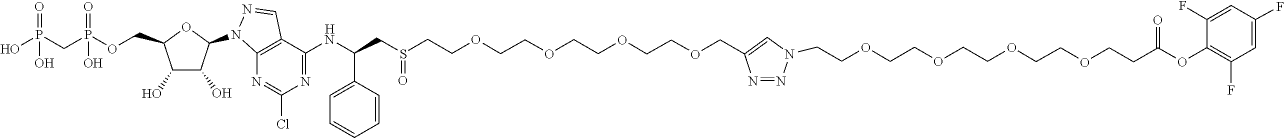 [SMILES: O=C(CCOCCOCCOCCOCCn1cc(COCCOCCOCCOCCS(=O)C[C@H](Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F]

To a solution of product from the previous step (53 mg, 0.064 mmol) and azido intermediate A (35 mg, 0.083 mmol), dissolved in DMF (1 mL), was added a solution of copper(II)sulfate (2.6 mg, 0.0160 mmol), sodium ascorbate (12.7 mg, 0.064 mmol), and THPTA (11.1 mg, 0.026 mmol) dissolved in water (0.5 mL). After stirring at room temperature for 20 minutes, LCMS shows complete conversion to product. The reaction was purified directly by RPLC (10% acetonitrile/water to 100% acetonitrile with 0.1% TFA). Yield 49 mg, 64%. Ions found by LCMS: [(M+2H)/2]⁺=624.3

### Synthesis of Conjugate 10

The title conjugate was prepared analogously to Conjugate 2, where the trifluorophenyl ester from Int-2 was replaced with the trifluorophenyl ester from Int-14.

### Synthesis of Int-13

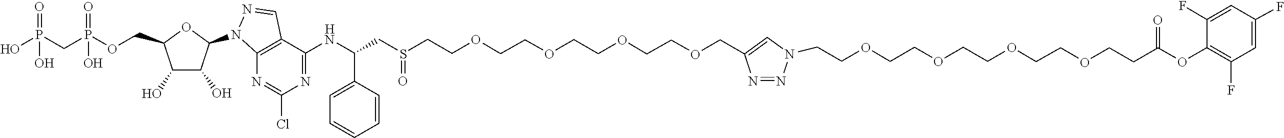 [SMILES: O=C(CCOCCOCCOCCOCCn1cc(COCCOCCOCCOCCS(=O)C[C@@H](Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F]

The title compound was prepared analogously to Int-14, where the alkyne thioether intermediate was replaced with the analogous alkyne thioether intermediate from Int-1.

### Synthesis of Conjugate 9

The title conjugate was prepared analogously to Conjugate 2, where the trifluorophenyl ester from Int-2 was replaced with the trifluorophenyl ester from Int-13.

### Synthesis of Int-33

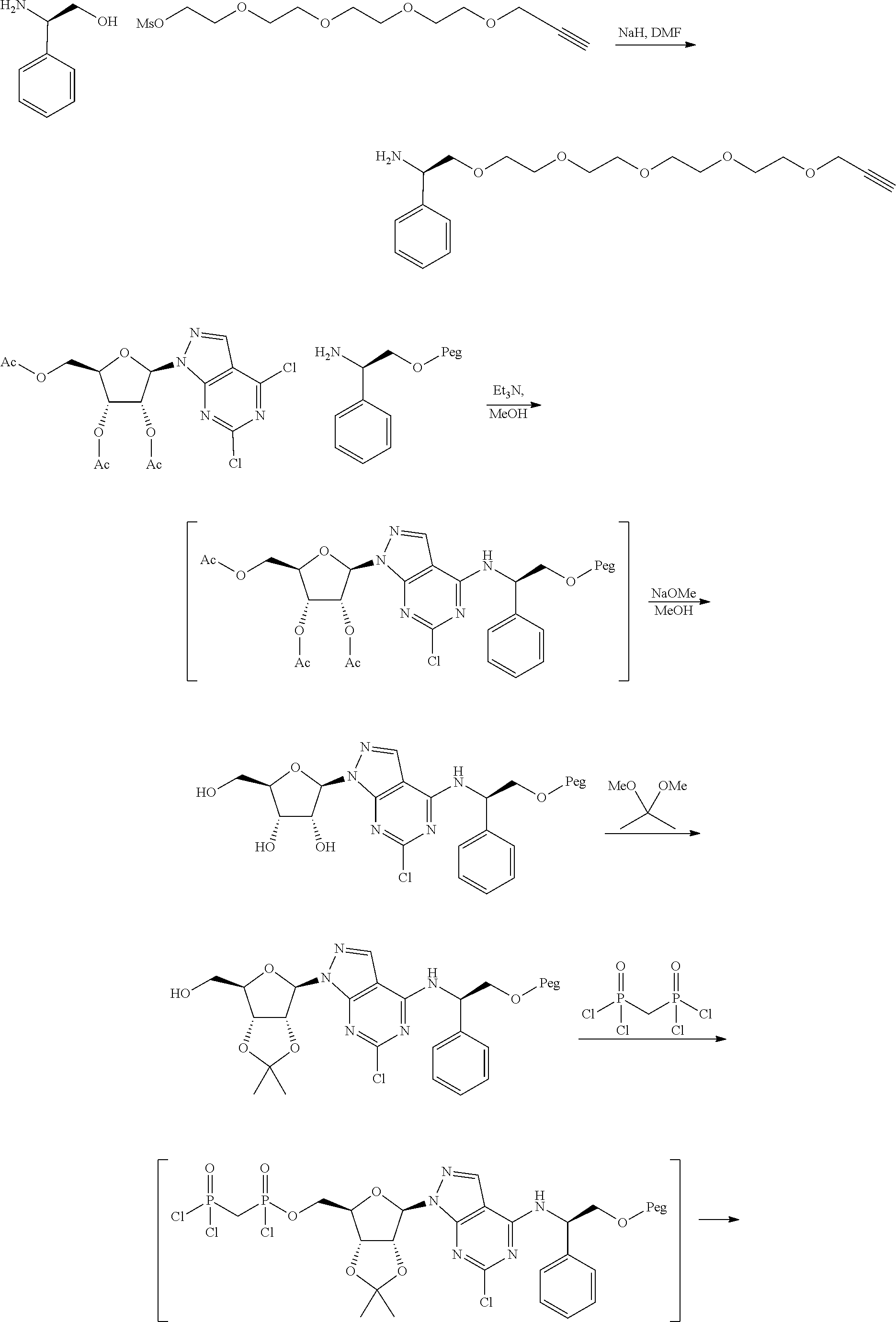

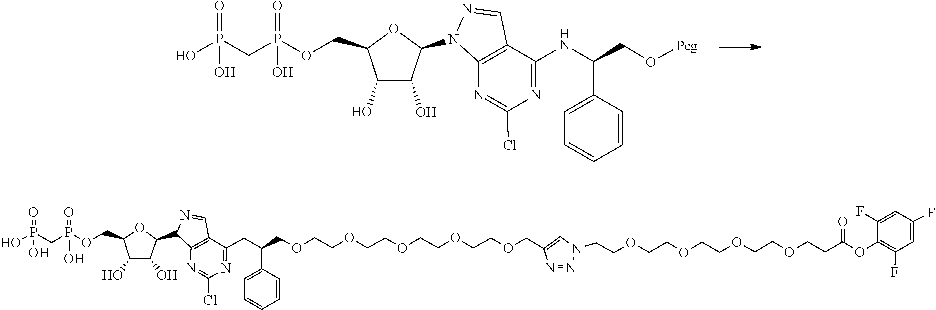

### Step a

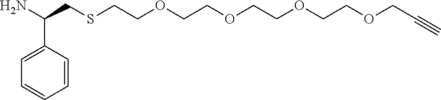 [SMILES: C#CCOCCOCCOCCOCCSC[C@H](N)c1ccccc1]

A solution of R-phenyl glycinol (411 mg, 3.00 mmol), dissolved in DMF (5 mL), was treated with sodium hydride (156 mg, 3.89 mmol, 60% in mineral oil) at room temperature for 10 minutes. To this solution was added propargyl-Peg4-mesylate (1.12 g, 3.60 mmol) in one portion. LCMS after 30 minutes showed complete conversion to product. The reaction was neutralized with glacial acetic acid, concentrated, and purified by RPLC (10% acetonitrile/water to 100% acetonitrile with 0.1% TFA). Yield of mono TFA salt is 1.18 g, 85%. Ions found by LCMS: [M+H]⁺=352.2

### Step b

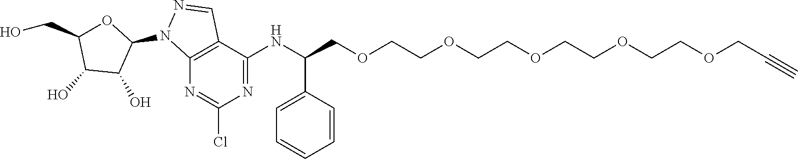 [SMILES: C#CCOCCOCCOCCOCCOC[C@H](Nc1nc(Cl)nc2c1cnn2[C@@H]1O[C@H](CO)[C@@H](O)[C@H]1O)c1ccccc1]

Product from the previous step (364 mg, 0.78 mmol), intermediate B (350 mg, 0.78 mmol), and DIEA (0.545 mL, 3.13 mmol), were mixed in methanol (4 mL) at ambient temperature. LCMS after 5 minutes shows ˜90% conversion to product. LCMS after 1 hour shows complete consumption of starting materials. Sodium methoxide (1.07 mL, 4.70 mmol, 25% sodium methoxide in methanol) was added at ambient temperature. LCMS after 10 minutes showed complete deprotection of acetates. Reaction was neutralized with glacial acetic acid, concentrated, and purified by RPLC (10% acetonitrile/water to 100% acetonitrile with 0.1% TFA). Yield 330 mg, 66%. Ions found by LCMS: [M+H]⁺=635.8

### Step c

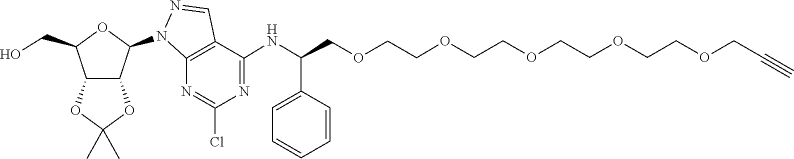 [SMILES: C#CCOCCOCCOCCOCCOC[C@H](Nc1nc(Cl)nc2c1cnn2[C@@H]1O[C@H](CO)[C@H]2OC(C)(C)O[C@H]21)c1ccccc1]

Product from the previous step (330 mg, 0.519 mmol), dissolved in acetone (3 mL), was treated with dimethoxy propane (3 mL) and toluene sulfonic acid (18 mg, 0.104 mmol). LCMS after 1h showed complete conversion to acetonide. Work-up: The reaction was neutralized with DIEA, concentrated, and purified by flash chromatography (0% MeOH/DCM to 10% MeOH/DCM). Yield 262 mg, 75%. Ions found by LCMS: [M+H]⁺=676.2

### Step d

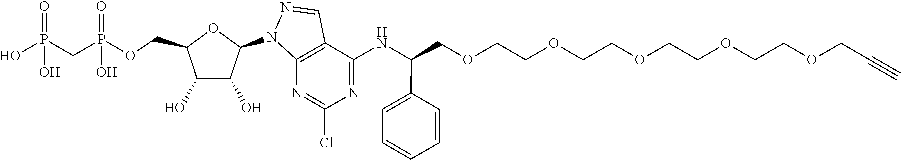 [SMILES: C#CCOCCOCCOCCOCCOC[C@H](Nc1nc(Cl)nc2c1cnn2[C@@H]1O[C@H](COP(=O)(O)CP(=O)(O)O)[C@@H](O)[C@H]1O)c1ccccc1]

Product from the previous step (262 mg, 0.387 mmol), dissolved in THE (3 mL), was treated with methylene (bis phosphonic dichloride) (387 mg, 1.55 mmol) at ambient temperature under an atmosphere of nitrogen. LCMS shows that all starting material had been consumed after 1.5 hour. The reaction was cooled with an ice bath then treated with 2M HCl (4 mL) and stirred at room temperature. LCMS after 1.5 hours showed complete acetonide deprotection. The reaction was concentrated by rotary evaporation, then purified by RPLC (10% acetonitrile/water to 100% acetonitrile with 0.1% TFA). Yield 161 mg, 52%. Ions found by LCMS: [M+H]⁺=794.2

### Step e

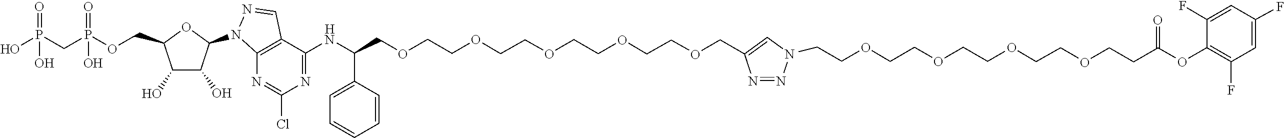 [SMILES: O=C(CCOCCOCCOCCOCCn1cc(COCCOCCOCCOCCOC[C@H](Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F]

To a solution of product from the previous step (161 mg, 0.203 mmol) and azido intermediate-A (111 mg, 0.264 mmol), dissolved in DMF (1 mL), was added a solution of copper(II)sulfate (8.1 mg, 0.0507 mmol), sodium ascorbate (40.2 mg, 0.203 mmol), and THPTA (35.2 mg, 0.0811 mmol) dissolved in water (0.5 mL). After stirring at ambient temperature for 20 minutes, LCMS shows complete conversion to product. The reaction was purified directly by RPLC (10% acetonitrile/water to 100% acetonitrile with 0.1% TFA). Yield 224 mg, 91%. Ions found by LCMS: [(M+2H)/2]⁺=608.2

### Synthesis of Conjugate 20

A solution of trifluorophenyl ester (8.4 mg, 0.0084 mmol, Int-33), dissolved in DMF (1 mL), was added to a solution of Fc (50 mg, 0.00086 mmol, SEQ ID NO: 13 in PBS at pH 7.4, 19.5 mg/mL) at room temperature. The pH of the resulting solution was adjusted to ˜8.5 with borate buffer (300 uL, 1M, pH 8.5). The homogeneous colorless reaction was rocked gently for 3 h, then submitted for purification according to the general procedure. Maldi TOF analysis of the purified final product gave an average mass of 62,315 Da (DAR=3.8). Yield: 69.6 mg, 79%.

### Synthesis of Int-34

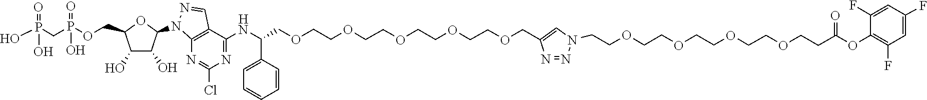 [SMILES: O=C(CCOCCOCCOCCOCCn1cc(COCCOCCOCCOCCOC[C@@H](Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F]

The title compound was prepared analogously to Int-33, where (R) phenyl glycinol was replaced with (S) phenyl glycinol in step a of that example.

### Synthesis of Conjugate 29

The title conjugate was prepared analogously Conjugate 20, where the trifluorophenyl ester from Int-33 was replaced with the trifluorophenyl ester from Int-34.

### Synthesis of Int-62

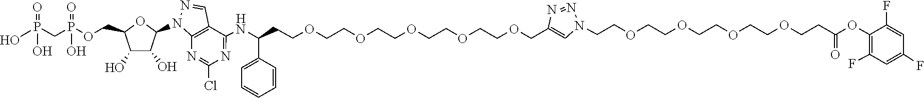 [SMILES: O=C(CCOCCOCCOCCOCCn1cc(COCCOCCOCCOCCOCC[C@H](Nc2nc(Cl)nc3c2cnn3[C@@H]2O[C@H](COP(=O)(O)CP(=O)(O)O)[C@@H](O)[C@H]2O)c2ccccc2)nn1)Oc1c(F)cc(F)cc1F]

The title compound was prepared analogously to Int-33, where (R) phenyl glycinol was replaced with (3S)-3-Amino-3-phenylpropan-1-ol in step a of that example.

### Synthesis of Conjugate 34

A solution of trifluorophenyl ester (10.4 mg, 0.0077 mmol, Int-62, dissolved in DMF (1 mL), was added to a solution of Fc (50 mg, 0.00086 mmol, SEQ ID NO: 13 in PBS at pH 7.4, 19.5 mg/mL) at ambient temperature. The pH of the resulting solution was adjusted to ˜8.5 with borate buffer (300 uL, 1M, pH 8.5). The homogeneous colorless reaction was rocked gently for 3 h, then submitted for purification according to the general procedure. Maldi TOF analysis of the purified final product gave an average mass of 63,518 Da (DAR=4.9). Yield: 41 mg, 82%.

### Synthesis of Int-22

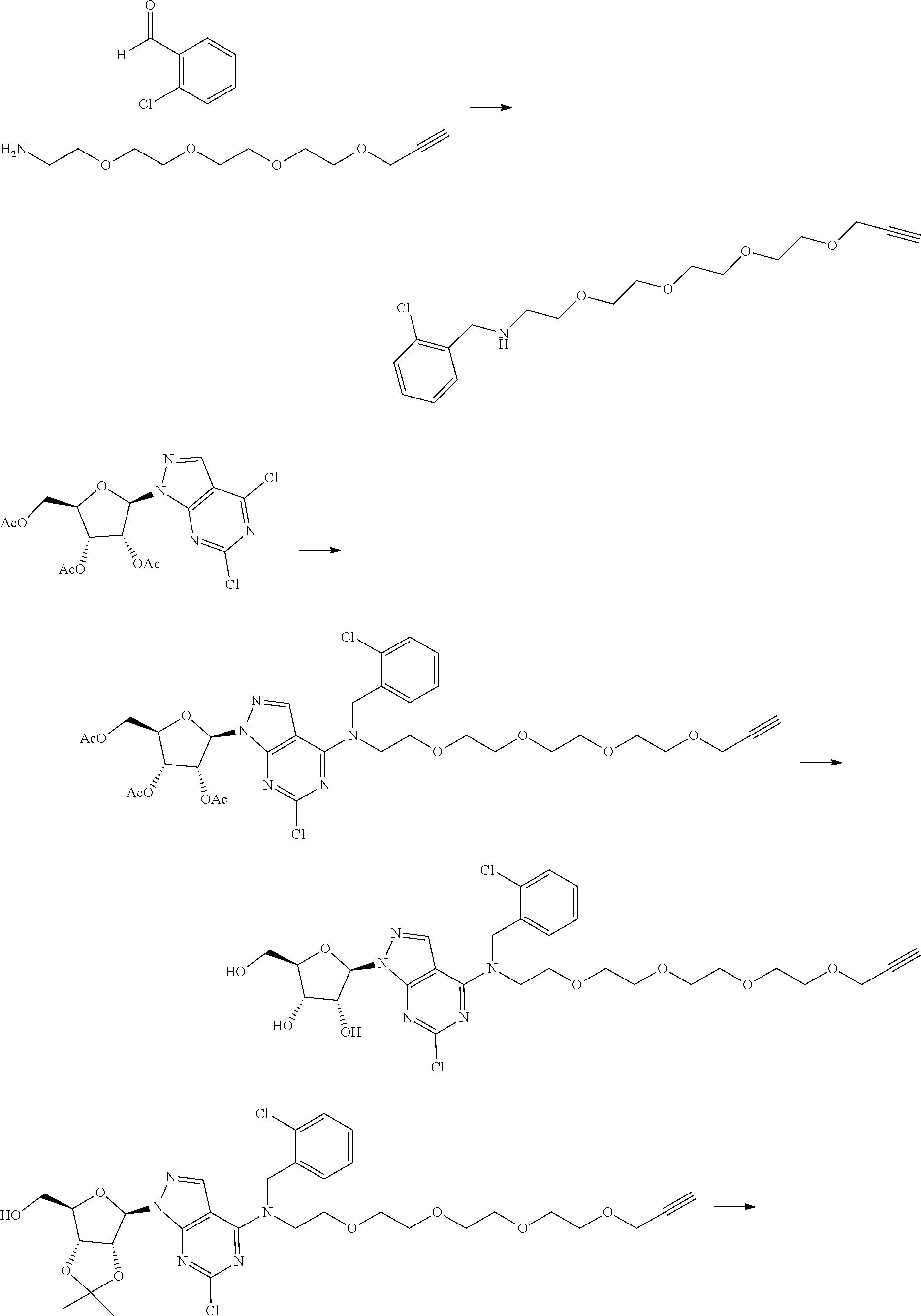

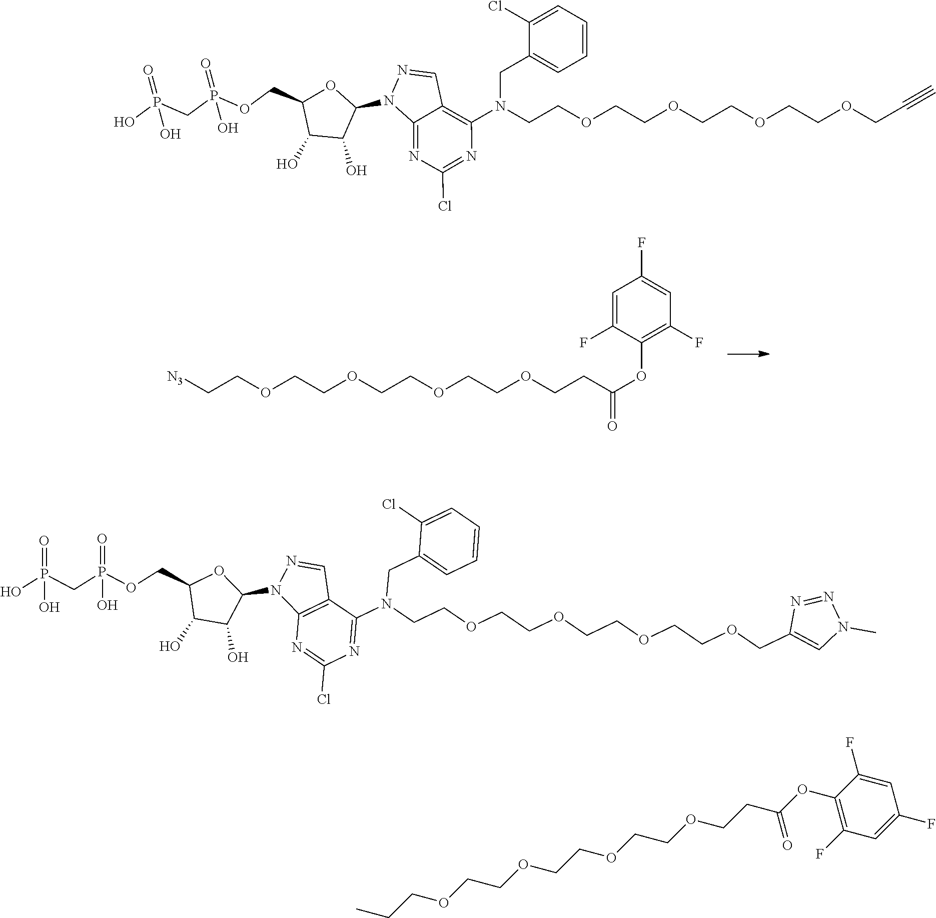

### Step a

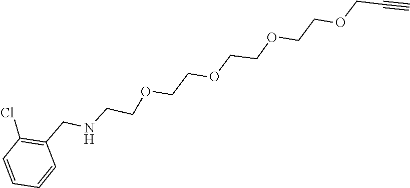 [SMILES: C#CCOCCOCCOCCOCCNCc1ccccc1Cl]

Propargyl-Peg4-amine (450 mg, 1.95 mmol) and 2-chloro-benzaldehyde (274 mg, 1.95 mmol) were stirred in methanol (10 mL) for 16 hours at ambient temperature. To this was added sodium borohydride (296 mg, 7.8 mmol) and the solution was stirred for an additional 1 hour. The excess methanol was removed by the rotary evaporator and the solution was partitioned between 1 N NaOH (50 mL) and ethyl acetate (50 mL). The aqueous layer was discarded, excess ethyl acetate was removed, and the crude mixture was purified by reversed phase flash chromatography (0% to 100% CH₃CN/H₂O). Yield: 346 mg, 50%. Ion(s) found by LCMS: [M+H]⁺=356.2.

### Step b

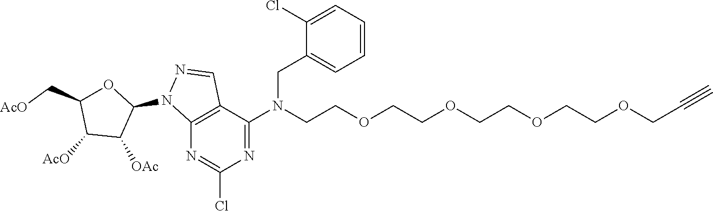 [SMILES: C#CCOCCOCCOCCOCCN(Cc1ccccc1Cl)c1nc(Cl)nc2c1cnn2[C@@H]1O[C@H](COC(C)=O)[C@@H](OC(C)=O)[C@H]1OC(C)=O]

Intermediate B (437 mg, 0.98 mmol), the product from the previous step (346 mg, 0.98 mmol) and DIEA (405 mg, 2.94 mmol) were stirred in ethanol (25 mL) at 50° C. for 4 hours. The mixture was cooled to ambient temperature and concentrated. The crude mixture was purified by reversed phase flash chromatography (0% to 100% CH₃CN/H₂O). Yield 204 mg, 27%. L^{C}/MS [M+H]+=753.2.

### Step c

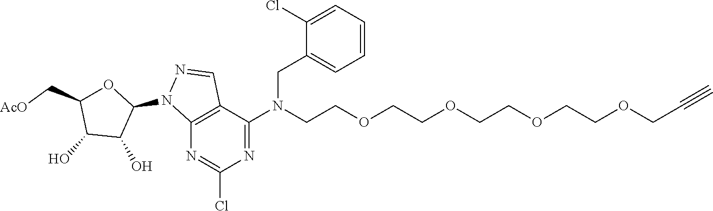 [SMILES: C#CCOCCOCCOCCOCCN(Cc1ccccc1Cl)c1nc(Cl)nc2c1cnn2[C@@H]1O[C@H](COC(C)=O)[C@@H](O)[C@H]1O]

To a solution of the triacetate (204 mg, 0.27 mmol) in methanol (20 mL) was added sodium methoxide 25% in methanol (2 mL). The solution was stirred for 2 hours at ambient temperature. The crude mixture was neutralized with glacial acetic acid (4 mL) and then concentrated and purified by semi-preparative HPLC (0% to 100% CH₃CN/H₂O, 0.1% TFA modifier). Yield 154 mg, 89%. Ion(s) found by L^{C}/MS [M+H]+=640.2.

### Step d

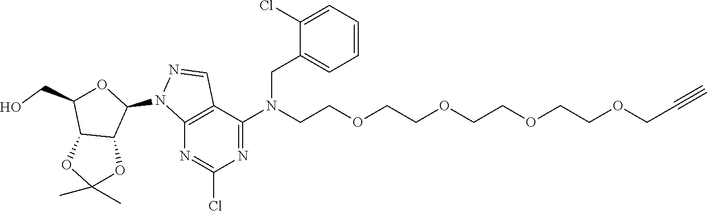 [SMILES: C#CCOCCOCCOCCOCCN(Cc1ccccc1Cl)c1nc(Cl)nc2c1cnn2[C@@H]1O[C@H](CO)[C@H]2OC(C)(C)O[C@H]21]

To a solution of the triol from the previous step (154 mg, 0.24 mmol) in acetone (20 mL) was added 2,2-dimethoxypropane (1.69 g, 16.29 mmol) and TsOH (10 mg, 0.05 mmol). The solution was stirred for 16 hours at ambient temperature. The excess TsOH was neutralized with DIEA (1 mL) and the crude mixture was concentrated and purified by semi-preparative HPLC (0% to 100% CH₃CN/H₂O, 0.1% TFA modifier).

Yield 110 mg, 67%. Ion(s) found by L^{C}/MS [M+H]+=680.2.

### Step e

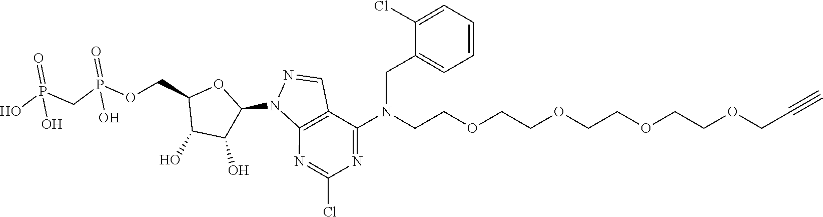 [SMILES: C#CCOCCOCCOCCOCCN(Cc1ccccc1Cl)c1nc(Cl)nc2c1cnn2[C@@H]1O[C@H](COP(=O)(O)CP(=O)(O)O)[C@@H](O)[C@H]1O]

The acetonide from the previous step (110 mg, 0.161 mmol) in THE (6 mL) was added, dropwise, to a mixture of methylene (bis phosphonic dichloride) (121 mg, 0.485 mmol) and DIEA (31 mg, 0.241 mmol) in THE (10 mL) cooled to 0° C. via an ice bath. When the addition was complete the ice bath was removed and the reaction was stirred for 3 hours at ambient temperature. The mixture was then cooled to 0° C. via an ice bath and 0.1 N aqueous HCl (30 mL) was added. The mixture was stirred at 0° C. for 15 min then at rt for 16 h (monitored by L^{C}/MS). The solvent was removed on the rotary evaporator and the crude mixture was purified by semi-preparative HPLC (0% to 100% CH₃CN/H₂O, 0.1% TFA modifier). The pure fractions were pooled and lyophilized to afford the product as a white, hygroscopic solid. Yield 44 mg, 34%. Ion(s) found by L^{C}/MS [M+H]+=798.1.

### Step f

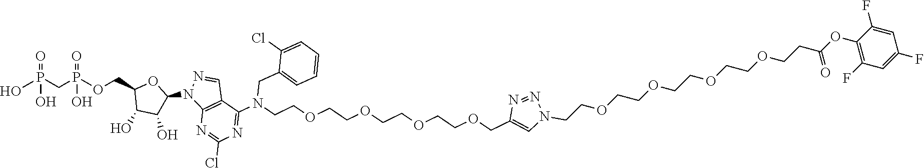 [SMILES: O=C(CCOCCOCCOCCOCCn1cc(COCCOCCOCCOCCN(Cc2ccccc2Cl)c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

The alkyne from step e (20 mg, 0.025 mmol) and intermediate A (18 mg, 0.042 mmol), were dissolved in DMF (1 mL) and cooled to 0° C. via an ice water bath. Premixed copper(II) sulfate (1 mg, 0.006 mmol) and sodium ascorbate (5 mg, 0.025 mmol) in DI water (1 mL) were then added and the mixture was stirred for 4 hours at ambient temperature. The crude reaction mixture was purified directly via semi-preparative HPLC (0% to 100% CH₃CN/H₂O, 0.1% TFA modifier). The pure fractions were pooled and lyophilized to afford the product as a white, hygroscopic solid. Yield 20 mg, 64%. Ion(s) found by L^{C}/MS [(M/2)+H]+=610.3.

### Synthesis of Conjugate 14a

To a solution of SEQ ID NO: 13 (3.40 mL, 100 mg, 0.0017 mmol) in PBS 7.4 was added Int-132 (21 mg, 0.017 mmol) in DMF (0.200 mL). The pH of the reaction mixture was slowly adjusted to ˜8.5 by the addition of 2 mL of 1M potassium carbonate buffer (pH 9). The reaction was then gently rocked for 4 hours. The reaction was quenched by stirring in a 150 mM His/100 mM ammonium hydroxide buffer (pH 8.5) for 12 hours and then submitted for purification according to the general procedure.

Trifluorophenol ester (described in synthesis of Int-22) was conjugated to Fc carrier SEQ ID NO: 13 as described in the general conjugation procedure using bicarbonate buffer (0.1-0.3 mL) to adjust the pH to ˜8.5. Yield 84.4%, average MALDI mass=62328 Da. (DAR=3.8).

### Synthesis of Int-46

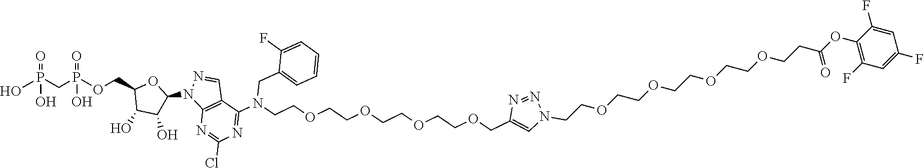 [SMILES: O=C(CCOCCOCCOCCOCCn1cc(COCCOCCOCCOCCN(Cc2ccccc2F)c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

The title compound was prepared analogously to Int-22. Ion(s) found by LCMS: [(M/2)+H]+=602.3.

### Synthesis of Conjugate 26

Trifluorophenol ester (described in synthesis of Int-22) was conjugated to Fc carrier SEQ ID NO: 13 as described in the general conjugation procedure using bicarbonate buffer (0.1-0.3 mL) to adjust the pH to ˜8.5. Yield 85.9%, average MALDI mass=62,252 Da. (DAR=3.8).

### Synthesis of Int-59

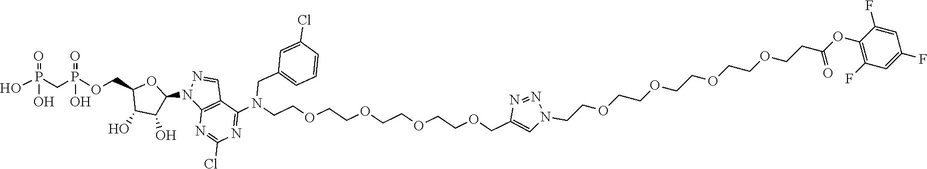 [SMILES: O=C(CCOCCOCCOCCOCCn1cc(COCCOCCOCCOCCN(Cc2cccc(Cl)c2)c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

The title compound was prepared analogously to Int-22. Ion(s) found by LCMS: [(M/2)+H]⁺=610.4.

### Synthesis of Conjugate 33

Trifluorophenol ester described in the synthesis of Int-59 was conjugated to Fc carrier SEQ ID NO: 13 as described in the synthesis of Conjugate 14a. Yield 77.8%, average MALDI mass=62,550 Da. (DAR=4.0).

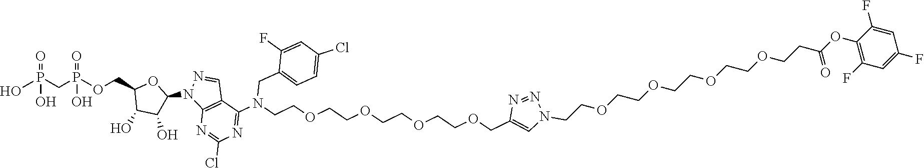 [SMILES: O=C(CCOCCOCCOCCOCCn1cc(COCCOCCOCCOCCN(Cc2ccc(Cl)cc2F)c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

### Synthesis of Int-73

### Synthesis of Conjugate 39

Trifluorophenol ester (described in the synthesis of Int-73) was conjugated to Fc carrier SEQ ID NO: 13 as described in the general conjugation procedure using bicarbonate buffer (0.1-0.3 mL) to adjust the pH to ˜8.5. Yield 86.6%, average MALDI mass=62439 Da. (DAR=3.9).

### Synthesis of Int-126

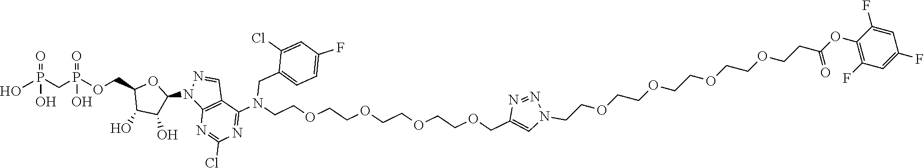 [SMILES: O=C(CCOCCOCCOCCOCCn1cc(COCCOCCOCCOCCN(Cc2ccc(F)cc2Cl)c2nc(Cl)nc3c2cnn3[C@@H]2O[C@H](COP(=O)(O)CP(=O)(O)O)[C@@H](O)[C@H]2O)nn1)Oc1c(F)cc(F)cc1F]

The title compound was prepared analogously to Int-22. Ion(s) found by LCMS: [(M/2)+H]⁺=619.3.

### Synthesis of Conjugate 67

Trifluorophenol ester (described in the synthesis of Int-126) was conjugated to Fc carrier SEQ ID NO: 13 as described in the general conjugation procedure using bicarbonate buffer (0.1-0.3 mL) to adjust the pH to ˜8.5. Yield 44.2%, average MALDI mass=64472 Da. (DAR=5.7).

### Synthesis of Int-93

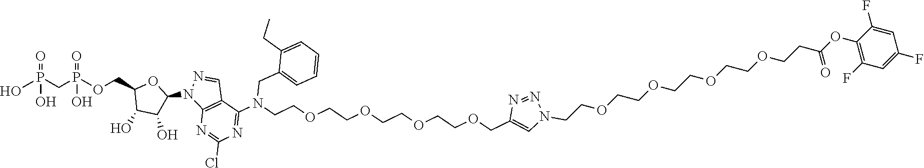 [SMILES: CCc1ccccc1CN(CCOCCOCCOCCOCc1cn(CCOCCOCCOCCOCCC(=O)Oc2c(F)cc(F)cc2F)nn1)c1nc(Cl)nc2c1cnn2[C@@H]1O[C@H](COP(=O)(O)CP(=O)(O)O)[C@@H](O)[C@H]1O]

The title compound was prepared analogously to Int-22. Ion(s) found by LCMS: [(M/2)+H]⁺=607.3.

### Synthesis of Conjugate 50

Trifluorophenol ester (described in the synthesis of Int-93) was conjugated to Fc carrier SEQ ID NO: 13 as described in the general conjugation procedure using bicarbonate buffer (0.1-0.3 mL) to adjust the pH to ˜8.5. Yield 72.8%, average MALDI mass=62455 Da. (DAR=4.0).

### Synthesis of Int-23

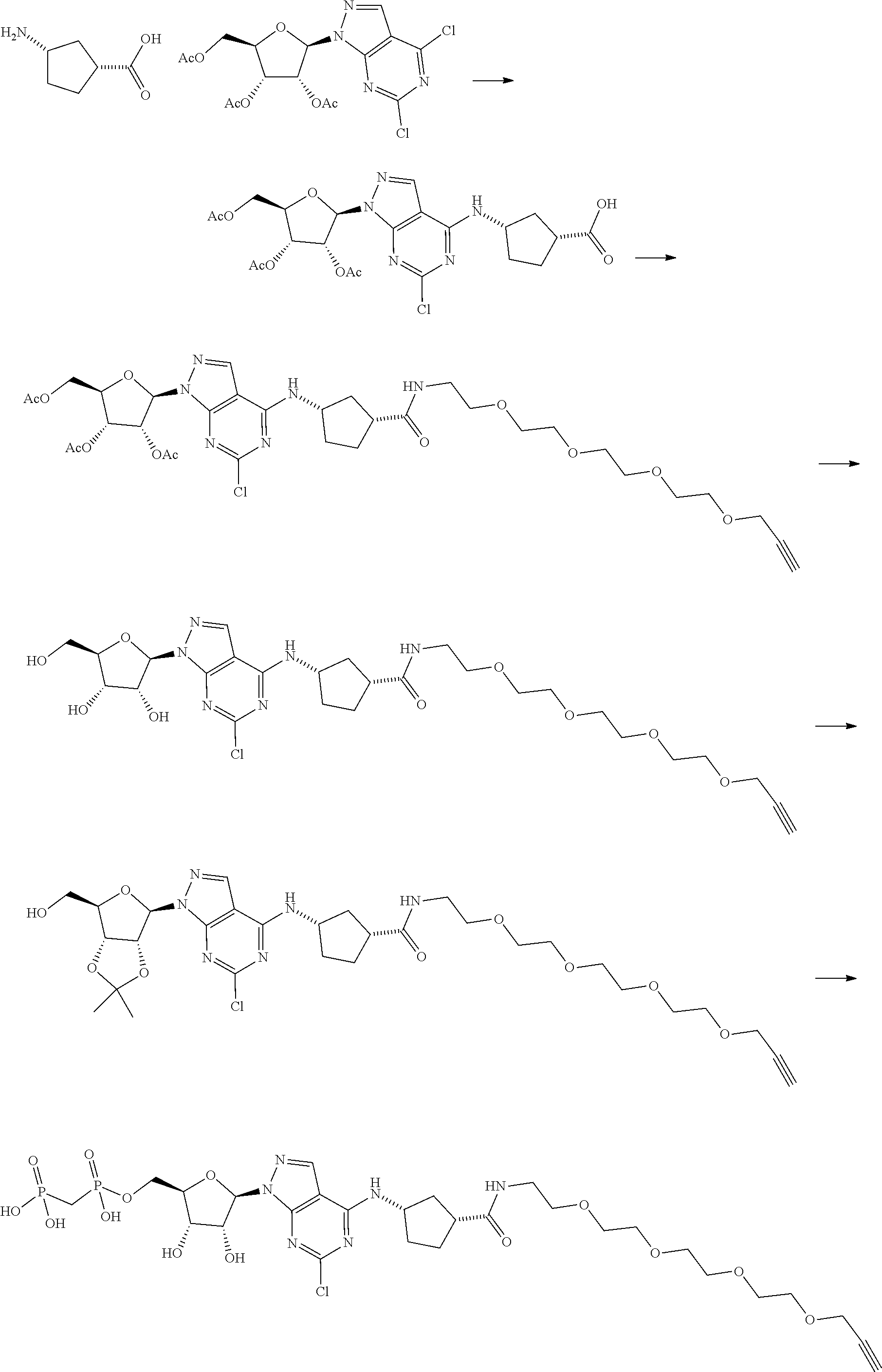

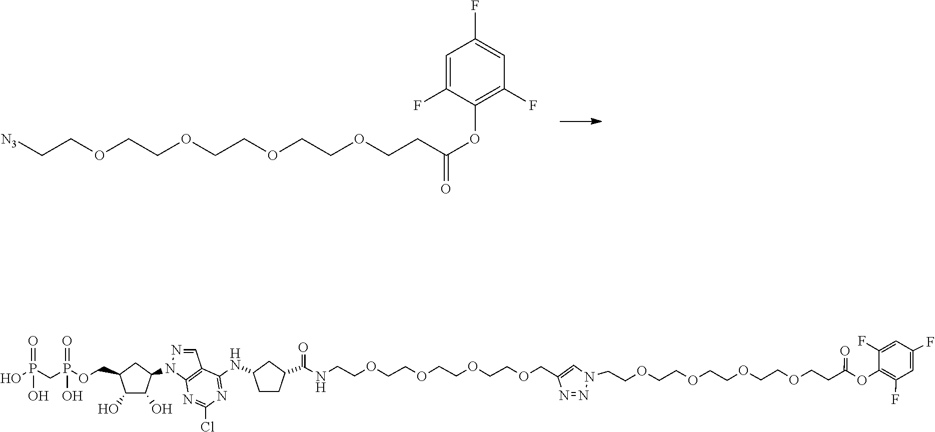

### Step a

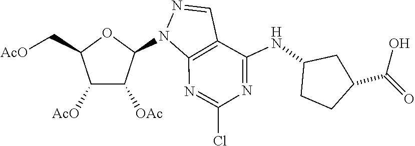

Intermediate B (1.10 g, 2.45 mmol), cis-3-aminocyclopentanecarboxylic acid (0.32 g, 2.45 mmol), and DIEA (0.64 g, 4.92 mmol) were stirred in ethanol (25 mL) at 50° C. for 4 hours. The mixture was cooled to ambient temperature and concentrated on a rotary evaporator. The crude mixture was purified by reversed phase flash chromatography (0% to 100% CH₃CN/H₂O). Yield: 740 mg, 55%. Ion(s) found by LCMS: [M+H]⁺=540.2.

### Step b

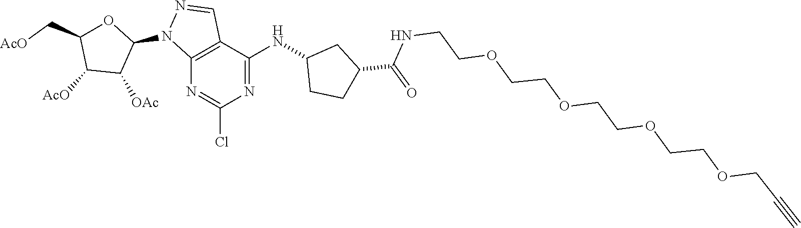

To a solution of the carboxylic acid from the previous step (740 mg, 1.37 mmol) in DMF (4 mL) was added propargyl-peg4-amine (0.32 g, 1.37 mmol) followed by DIEA (0.53 g, 4.10 mmol), and HATU (1.04 g, 2.73 mmol). The solution was stirred for 4 h at ambient temperature, then the crude material was purified by reversed phase flash chromatography (0% to 100% CH₃CN/H₂O). Yield: 1.02 g, 98%. Ion(s) found by LCMS: [M+H]⁺=753.2.

### Step c

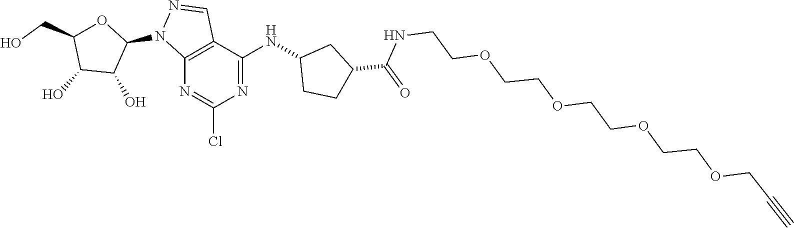

To a solution of triacetate from the previous step (1.02 g, 1.35 mmol) in MeOH (20 mL) was added sodium methoxide 25% in methanol (4 mL). The solution was stirred for 2 hours at ambient temperature. The crude mixture was neutralized with glacial acetic acid (6 mL) and the crude mixture was concentrated and purified by semi-preparative HPLC (0% to 100% CH₃CN/H₂O, 0.1% TFA modifier). Yield 560 mg, 68%. Ion(s) found by LC/MS [M+H]+=627.2.

### Step d

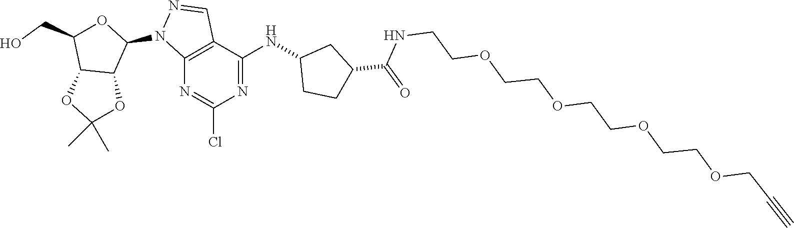

To a solution of the triol from the previous step (145 mg, 0.23 mmol) in acetone (10 mL) was added 2,2-dimethoxypropane (1.69 g, 16.29 mmol) and TsOH (10 mg, 0.05 mmol). The solution was stirred for 16 hours at ambient temperature. The excess TsOH was neutralized with DIEA base (1 mL) and the crude mixture was concentrated and purified by semi-preparative HPLC (0% to 100% CH₃CN/H₂O, 0.1% TFA modifier). Yield 82 mg, 54%. Ion(s) found by LC/MS [M+H]+=667.2.

### Step e

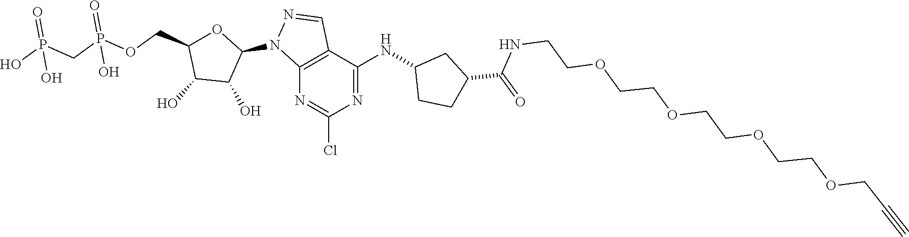

The acetonide from the previous step (82 mg, 0.124 mmol) in THE (6 mL) was added, dropwise to a mixture of methylene (bis phosphonic dichloride) (93 mg, 0.372 mmol) and DIEA (23 mg, 0.186 mmol) in THE (10 mL), cooled to 0° C. via an ice bath. When the addition was complete the ice bath was removed and the reaction was stirred for 3 hours at ambient temperature. The mixture was then cooled to 0° C. via an ice bath and 0.1 N aqueous HCl (30 mL) was added. The mixture was stirred at 0° C. for 15 min then at ambient temperature for 16 h (monitored by LC/MS). The solvent was removed and the crude mixture was purified by semi-preparative HPLC (0% to 100% CH₃CN/H₂O, 0.1% TFA modifier). The pure fractions were pooled and lyophilized to afford the product as a white, hygroscopic solid. Yield 42 mg, 43%. Ion(s) found by LC/MS [M+H]+=784.6.

### Step f

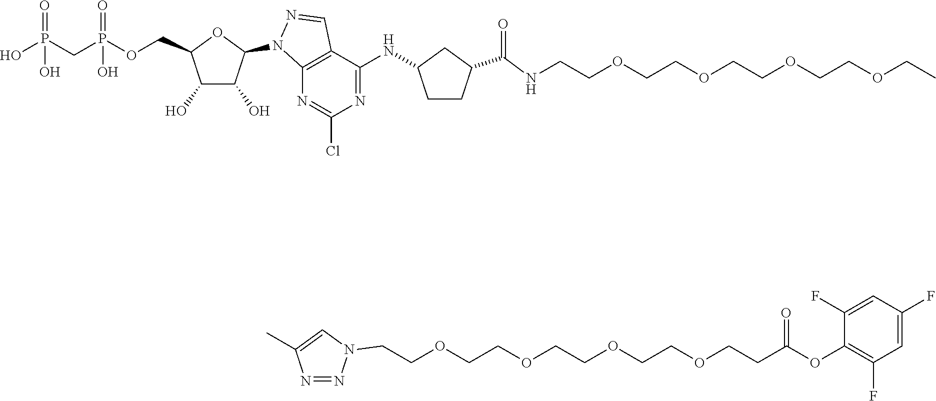

The alkyne from the previous step (20 mg, 0.025 mmol) and intermediate A (14 mg, 0.033 mmol), were dissolved in DMF (1 mL) and cooled to 0° C. via an ice water bath. Premixed copper(II) sulfate (1 mg, 0.006 mmol) and sodium ascorbate (5 mg, 0.025 mmol) in DI water (1 mL) were then added and the mixture was stirred for 4 hours at ambient temperature. The crude reaction mixture was purified directly via semi-preparative HPLC (0% to 100% CH₃CN/H₂O, 0.1% TFA modifier). The pure fractions were pooled and lyophilized to afford the product as a white, hygroscopic solid. Yield 13 mg, 43%. Ion(s) found by LC/MS [(M/2)+H]+=603.8.

### Synthesis of Conjugate 15

Trifluorophenol ester (described in the synthesis of Int-23) was conjugated to Fc carrier SEQ ID NO: 13 as described in the general conjugation procedure using bicarbonate buffer (0.1-0.3 mL) to adjust the pH to ˜8.5. Yield 83.7%, average MALDI mass=61,343 Da. (DAR=2.9).

### Synthesis of Int-128

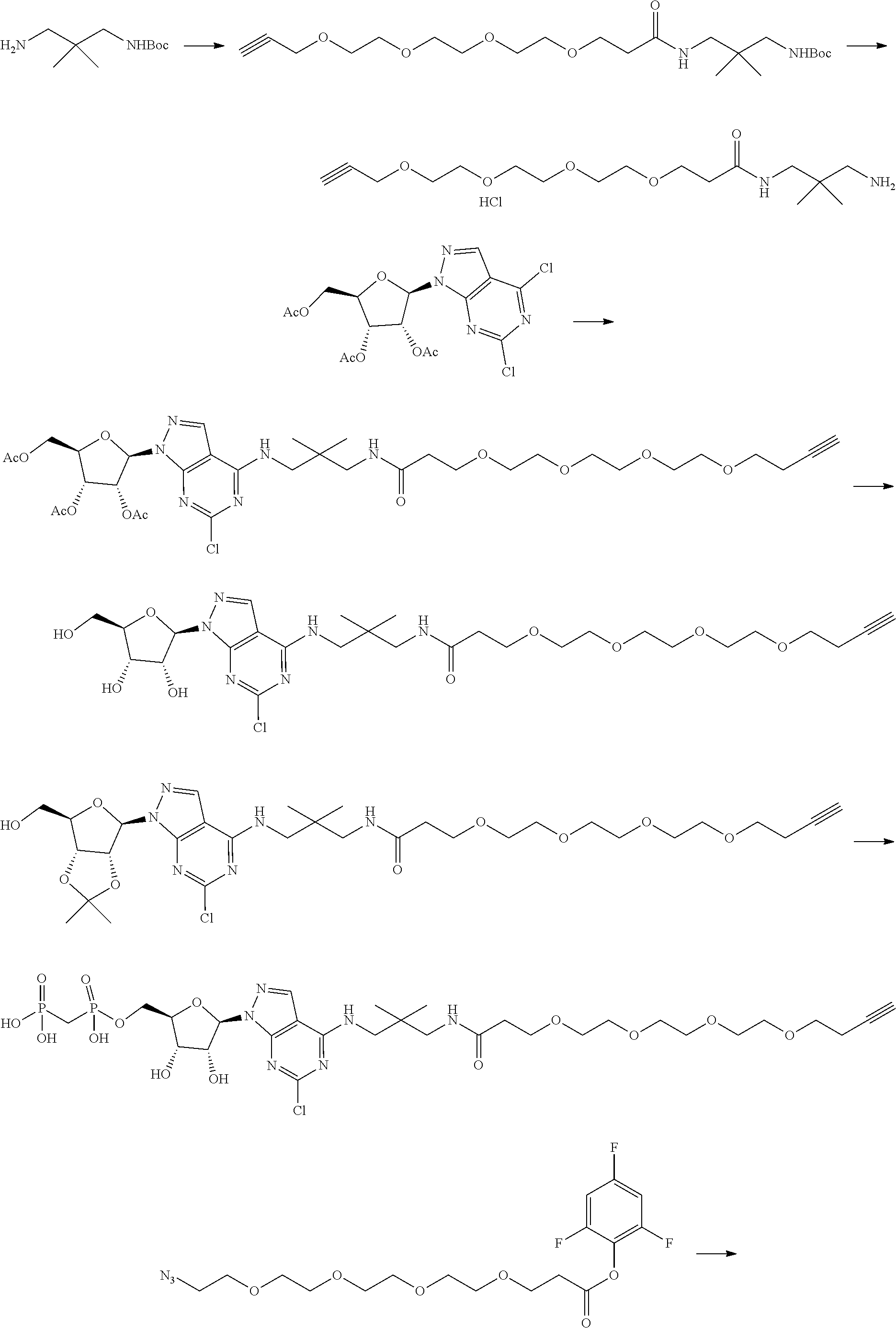

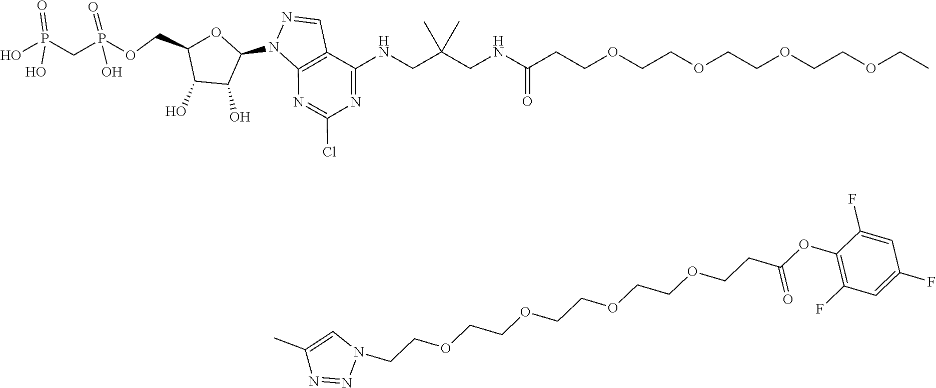

### Step a

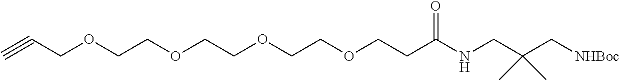 [SMILES: C#CCOCCOCCOCCOCCC(=O)NCC(C)(C)CNC(=O)OC(C)(C)C]

To a solution of N-(3-amino-2,2-dimethylpropyl)tert-butoxy)carboxamide (242 mg, 1.19 mmol) in DMF (3 mL) was added propargyl-Peg₄-carboxylic acid (311 mg, 1.19 mmol) and DIEA (463 mg, 3.59 mmol) followed by HATU (637 mg, 1.67 mmol). The solution was stirred for 16 hours at ambient temperature, then the crude material was purified directly by reversed phase flash chromatography (0% to 100% CH₃CN/H₂O). Yield 248 mg, 47%. Ion(s) found by LC/MS [M+H]+=445.4.

### Step b

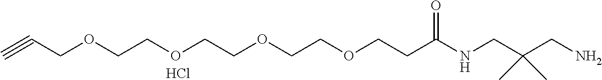 [SMILES: C#CCOCCOCCOCCOCCC(=O)NCC(C)(C)CN ; Cl]

To the product from the previous step (248 mg, 0.559 mmol) was added 4N HCl in dioxane (6 mL). The solution was stirred for 2 hours at ambient temperature. The excess dioxane was removed on the rotary evaporator and the crude material was used in the next reaction without additional purification. Ion(s) found by LC/MS [M+H]+=345.2.

### Step c

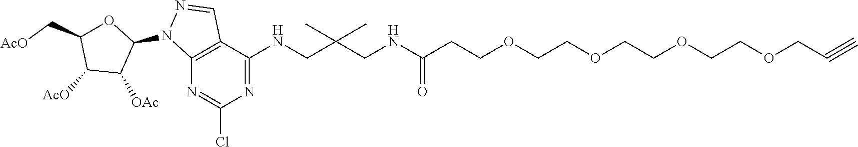 [SMILES: C#CCOCCOCCOCCOCCC(=O)NCC(C)(C)CNc1nc(Cl)nc2c1cnn2[C@@H]1O[C@H](COC(C)=O)[C@@H](OC(C)=O)[C@H]1OC(C)=O]

Intermediate B (250 mg, 0.55 mmol), the amine-HCl salt from the previous step (212 mg, 0.55 mmol) and DIEA (355 mg, 2.75 mmol) were stirred in ethanol (25 mL) at 50° C. for 4 h. The mixture was cooled to rt and concentrated. The crude mixture was purified by reversed phase flash chromatography (0% to 100% CH₃CN/H₂O). Yield 166 mg, 40%. Ion(s) found by LC/MS [M+H]+=755.2.

### Step d

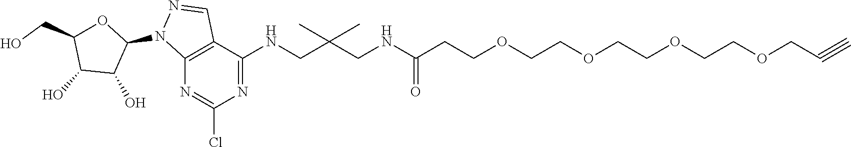 [SMILES: C#CCOCCOCCOCCOCCC(=O)NCC(C)(C)CNc1nc(Cl)nc2c1cnn2[C@@H]1O[C@H](CO)[C@@H](O)[C@H]1O]

To a solution of the triacetate (166 mg, 0.22 mmol) in MeOH (10 mL) was added NaOMe 25% in MeOH (2 mL). The solution was stirred for 2 h. The crude mixture was neutralized with AcOH (3 mL) and the crude mixture was concentrated and purified by semi-preparative HPLC (0% to 100% CH₃CN/H₂O, 0.1% TFA modifier). Yield 110 mg, 80%. Ion(s) found by LC/MS [M+H]+=629.2.

### Step e

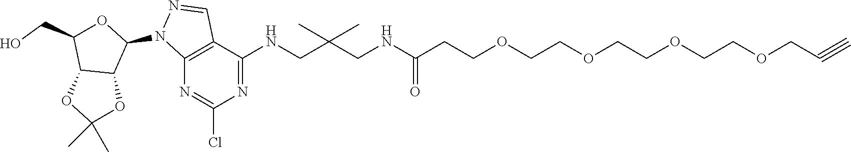 [SMILES: C#CCOCCOCCOCCOCCC(=O)NCC(C)(C)CNc1nc(Cl)nc2c1cnn2[C@@H]1O[C@H](CO)[C@H]2OC(C)(C)O[C@H]21]

To a solution of the triol from the previous step (110 mg, 0.18 mmol) in acetone (10 mL) was added 2,2-dimethoxypropane (1.69 g, 16.29 mmol) and TsOH (10 mg, 0.05 mmol). The solution was stirred for 16 hours at ambient temperature. The excess TsOH was neutralized with DIEA (1 mL) and the crude mixture was concentrated and purified by semi-preparative HPLC (0% to 100% CH₃CN/H₂O, 0.1% TFA modifier). Yield 58 mg, 48%. Ion(s) found by LC/MS [M+H]+=669.2.

### Step f

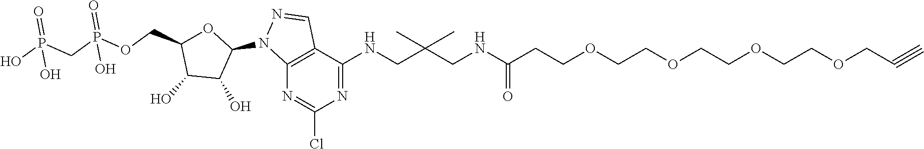 [SMILES: C#CCOCCOCCOCCOCCC(=O)NCC(C)(C)CNc1nc(Cl)nc2c1cnn2[C@@H]1O[C@H](COP(=O)(O)CP(=O)(O)O)[C@@H](O)[C@H]1O]

The acetonide from the previous step (58 mg, 0.087 mmol) in THE (6 mL) was added, dropwise, to a mixture of methylene (bis phosphonic dichloride) (65 mg, 0.261 mmol) and DIEA (16 mg, 0.131 mmol) in THE (10 mL), then cooled to 0° C. via an ice bath. When the addition was complete the ice bath was removed and the reaction was stirred for 3 hours at ambient temperature. The mixture was then cooled to 0° C. via an ice bath and 0.1 N aqueous HCl (30 mL) was added. The mixture was stirred at 0° C. for 15 min then at ambient temperature for 16 hours (monitored by LC/MS). The solvent was removed and the crude mixture was purified by semi-preparative HPLC (0% to 100% CH₃CN/H₂O, 0.1% TFA modifier). The pure fractions were pooled and lyophilized to afford the product as a white, hygroscopic solid. Yield 31 mg, 46%. Ion(s) found by LC/MS [M+H]+=787.2.

### Step g

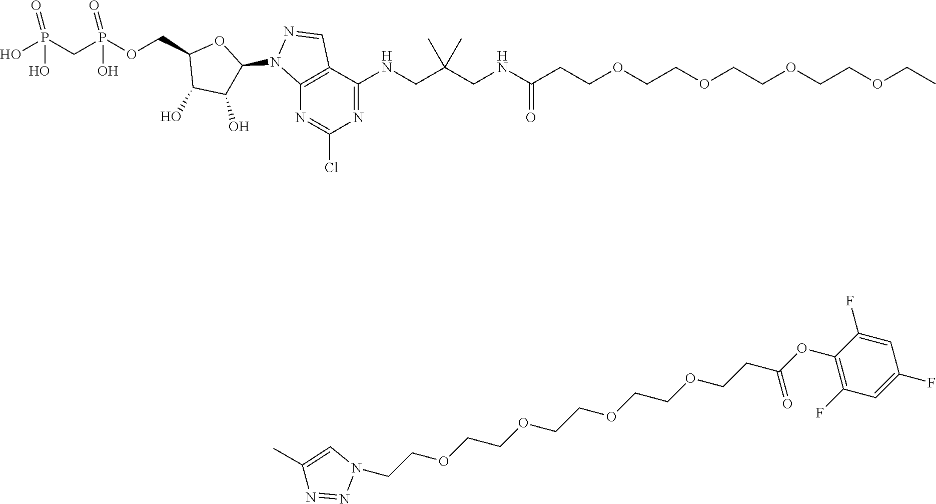

The alkyne from the previous step (20 mg, 0.025 mmol) and intermediate A (14 mg, 0.033 mmol), were dissolved in DMF (1 mL) and cooled to 0° C. via an ice water bath. Premixed copper(II) sulfate (1 mg, 0.006 mmol) and sodium ascorbate (5 mg, 0.025 mmol) in DI water (1 mL) were then added and the mixture was stirred for 4 h. The crude reaction mixture was purified directly via semi-preparative HPLC (0% to 100% CH₃CN/H₂O, 0.1% TFA modifier). The pure fractions were pooled and lyophilized to afford the product as a white, hygroscopic solid. Yield 15 mg, 51%. Ion(s) found by LC/MS [(M/2)+H]+=604.8.

### Synthesis of Conjugate 68

Trifluorophenol ester (described in the synthesis of Int-128) was conjugated to Fc carrier SEQ ID NO: 13 as described in the general conjugation procedure using bicarbonate buffer (0.1-0.3 mL) to adjust the pH to ˜8.5. Yield 76.5%, average MALDI mass=61839 Da. (DAR=3.4).

### Synthesis of Int-47

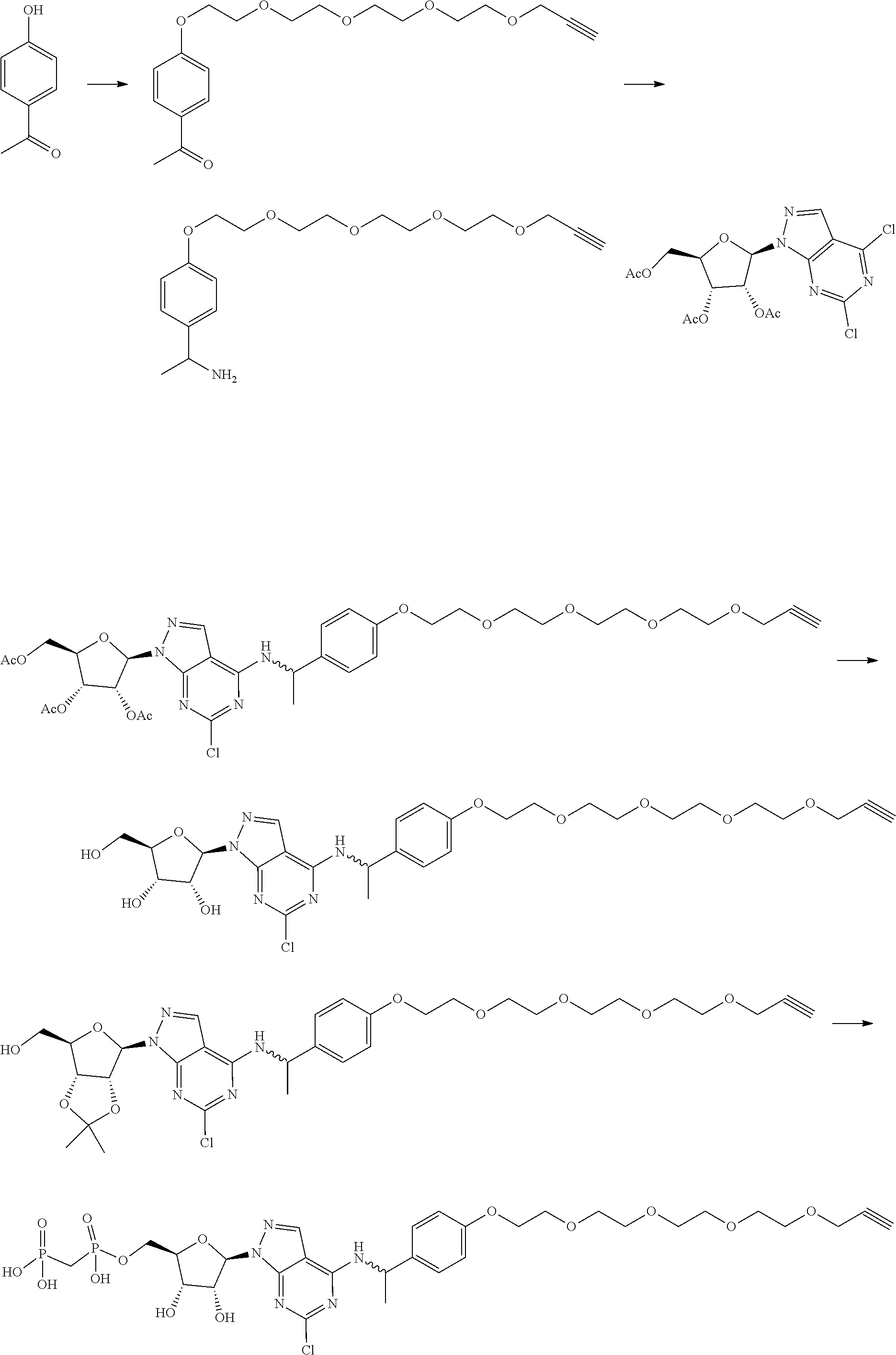

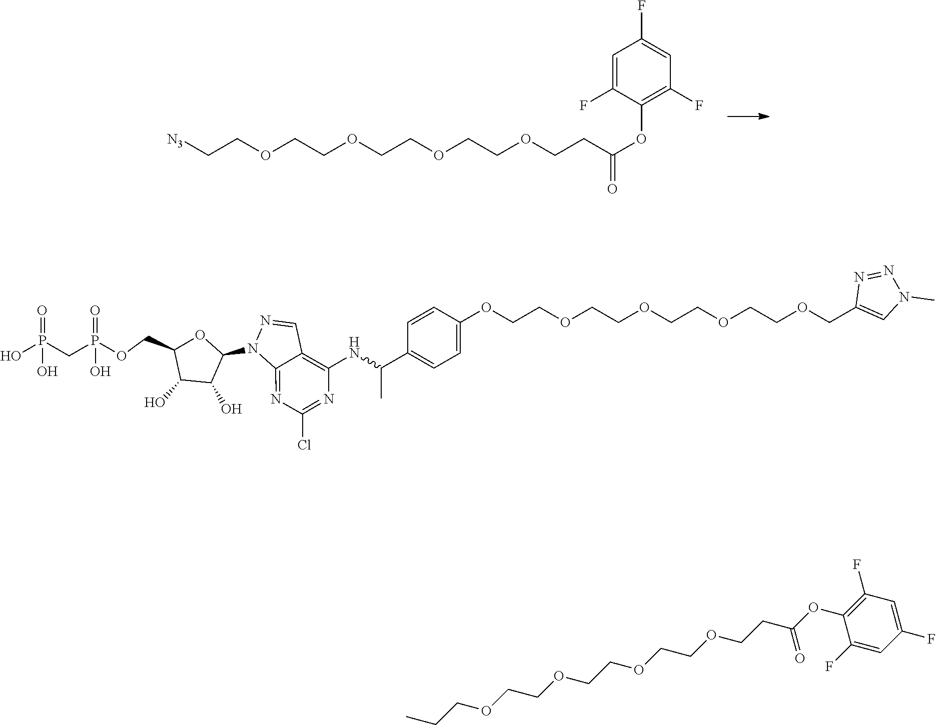

### Step a

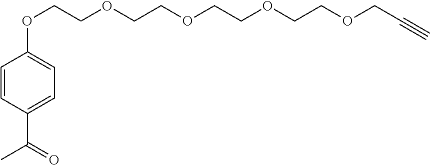 [SMILES: C#CCOCCOCCOCCOCCOc1ccc(C(C)=O)cc1]

To a solution of 4-hydroxy acetophenone (490 mg, 3.60 mmol) in CH₃CN (10 mL) was added cesium carbonate (1.75 g, 5.40 mmol) and the solution was stirred for 30 min. Propargyl-Peg₄ mesylate (1.1 g, 3.60 mmol) was then added and the solution was heated to 70° C. and stirred for 16 hours. The excess acetonitrile was removed and the crude material was purified by flash chromatography (0% to 100% EtOAc/hexanes). Yield: 43%, 550 mg. LC/MS [M+H]+=351.2.

### Step b

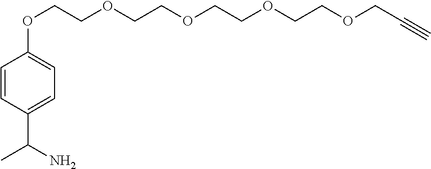 [SMILES: C#CCOCCOCCOCCOCCOc1ccc(C(C)N)cc1]

To a solution of the ketone from the previous step (550 mg, 1.57 mmol) in methanol (10 mL) was added ammonium acetate (4.8 g, 62.78 mmol) followed by sodium cyanoborohydride (197 mg, 3.13 mmol) the solution was stirred for 100 h. The excess methanol was removed and the crude mixture was purified by reversed phase flash chromatography (0% to 100% CH₃CN/H₂O). Yield 328 mg, 59%. Ion(s) found by LC/MS [M+H]+=352.2.

### Step c

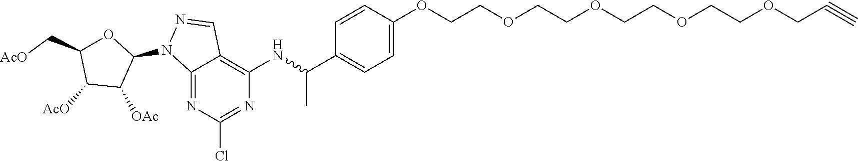 [SMILES: C#CCOCCOCCOCCOCCOc1ccc(C(C)Nc2nc(Cl)nc3c2cnn3[C@@H]2O[C@H](COC(C)=O)[C@@H](OC(C)=O)[C@H]2OC(C)=O)cc1]

Intermediate B (416 mg, 0.93 mmol), the amine from the previous step (328 mg, 0.93 mmol) and DIEA (359 mg, 2.79 mmol) were stirred in ethanol (25 mL) at 50° C. for 4 hours. The mixture was cooled to ambient temperature and concentrated on the rotary evaporator. The crude mixture was purified by reversed phase flash chromatography (0% to 100% CH₃CN/H₂O). Yield 403 mg, 57%. Ion(s) found by LC/MS [M+H]+=762.2.

### Step d

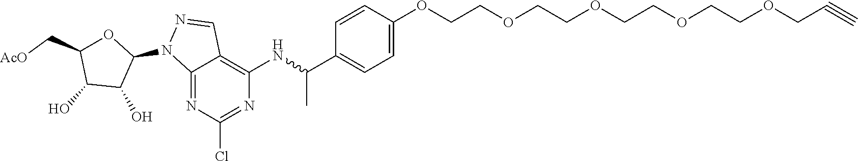 [SMILES: C#CCOCCOCCOCCOCCOc1ccc(C(C)Nc2nc(Cl)nc3c2cnn3[C@@H]2O[C@H](COC(C)=O)[C@@H](O)[C@H]2O)cc1]

To a solution of the triacetate from the previous step (403 mg, 0.53 mmol) in methanol (10 mL) was added sodium methoxide 25% in methanol (2 mL). The solution was stirred for 2 hours at ambient temperature. The crude mixture was neutralized with glacial acetic acid (3 mL) and the crude mixture was concentrated and purified by semi-preparative HPLC (0% to 100% CH₃CN/H₂O, 0.1% TFA modifier). Yield 141 mg, 42%. Ion(s) found by LC/MS [M+H]+=636.2.

### Step e

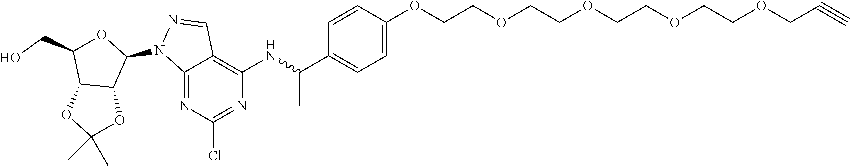 [SMILES: C#CCOCCOCCOCCOCCOc1ccc(C(C)Nc2nc(Cl)nc3c2cnn3[C@@H]2O[C@H](CO)[C@H]3OC(C)(C)O[C@H]32)cc1]

To a solution of the triol from the previous step (141 mg, 0.22 mmol) in acetone (10 mL) was added 2,2-dimethoxypropane (1.69 g, 16.29 mmol) and TsOH (10 mg, 0.05 mmol). The solution was stirred for 16 hours at ambient temperature. The excess TsOH was neutralized with DIEA (1 mL) and the crude mixture was concentrated and purified by semi-preparative HPLC (0% to 100% CH₃CN/H₂O, 0.1% TFA modifier). Yield 120 mg, 80%. Ion(s) found by LC/MS [M+H]+=676.2.

### Step f

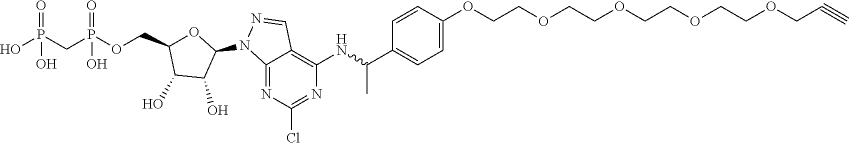 [SMILES: C#CCOCCOCCOCCOCCOc1ccc(C(C)Nc2nc(Cl)nc3c2cnn3[C@@H]2O[C@H](COP(=O)(O)CP(=O)(O)O)[C@@H](O)[C@H]2O)cc1]

The acetonide from the previous step (120 mg, 0.18 mmol) in THE (6 mL) was added, dropwise, to a mixture of methylene (bis phosphonic dichloride) (132 mg, 0.53 mmol) and DIEA (35 mg, 0.27 mmol) in THE (10 mL) cooled to 0° C. via an ice bath. When the addition was complete the ice bath was removed and the reaction was stirred for 3 hours at ambient temperature. The mixture was then cooled to 0° C. via an ice bath and 0.1 N aqueous HCl (30 mL) was added. The mixture was stirred at 0° C. for 15 min then at ambient temperature for 16 hours (monitored by LC/MS). The solvent was removed and the crude mixture was purified by semi-preparative HPLC (0% to 100% CH₃CN/H₂O, 0.1% TFA modifier). The pure fractions were pooled and lyophilized to afford the product as a white, hygroscopic solid. Yield 45 mg, 31%. Ion(s) found by LC/MS [M+H]+=794.2.

### Step q

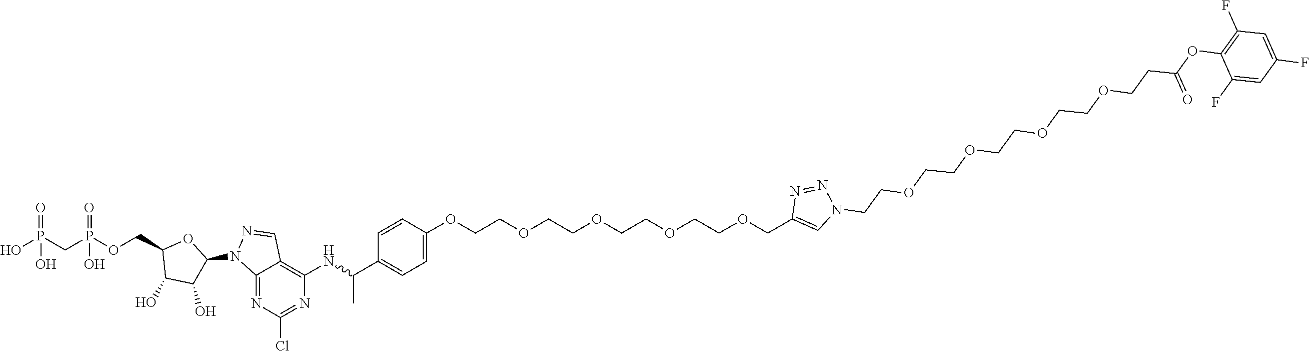 [SMILES: CC(Nc1nc(Cl)nc2c1cnn2[C@@H]1O[C@H](COP(=O)(O)CP(=O)(O)O)[C@@H](O)[C@H]1O)c1ccc(OCCOCCOCCOCCOCc2cn(CCOCCOCCOCCOCCC(=O)Oc3c(F)cc(F)cc3F)nn2)cc1]

The alkyne from the previous step (45 mg, 0.056 mmol) and intermediate A (31 mg, 0.074 mmol), were dissolved in DMF (1 mL) and cooled to 0° C. via an ice water bath. Premixed copper(II) sulfate (3 mg, 0.020 mmol) and sodium ascorbate (11 mg, 0.056 mmol) in DI water (1 mL) were then added, then mixture was stirred for 4 hours at ambient temperature. The crude reaction mixture was purified directly via semi-preparative HPLC (0% to 100% CH₃CN/H₂O, 0.1% TFA modifier). The pure fractions were pooled and lyophilized to afford the product as a white, hygroscopic solid. Yield 30 mg, 45%. Ion(s) found by LC/MS [(M/2)+H]+=608.4.

### Synthesis of Conjugate 27

Trifluorophenol ester (described in the synthesis of Int-47) was conjugated to Fc carrier SEQ ID NO: 13 as described in the general conjugation procedure using bicarbonate buffer (0.1-0.3 mL) to adjust the pH to ˜8.5. Yield 85.9%, average MALDI mass=61811 Da. (DAR=3.4)

### Synthesis of Int-87

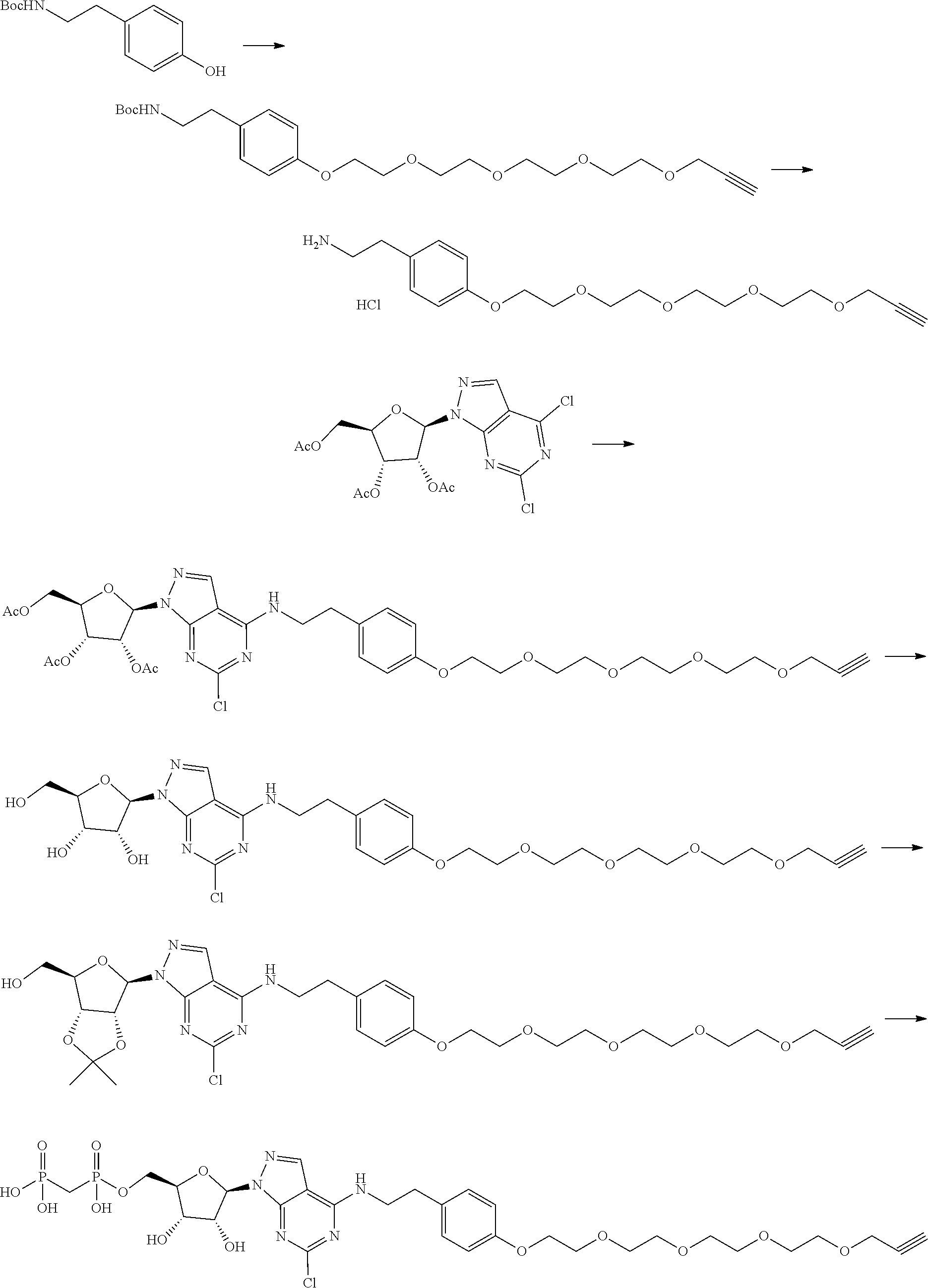

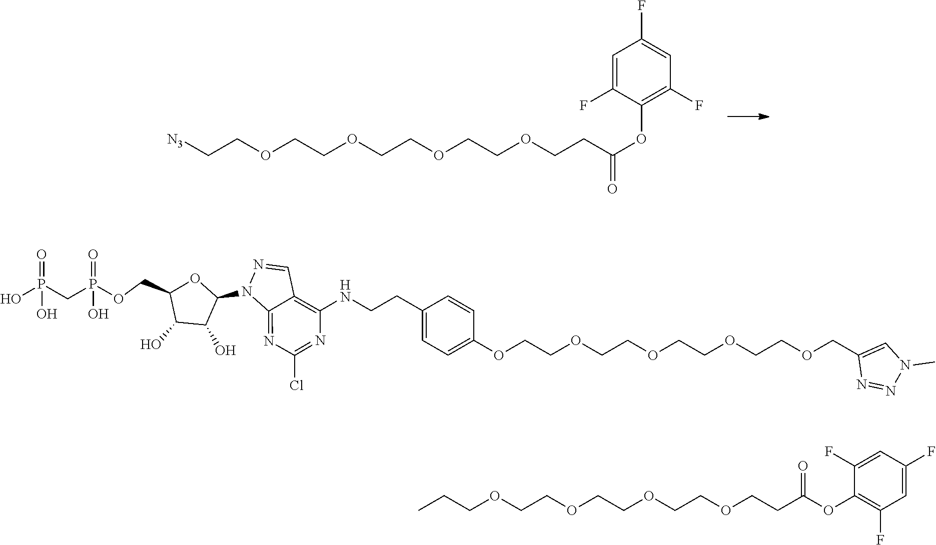

### Step a

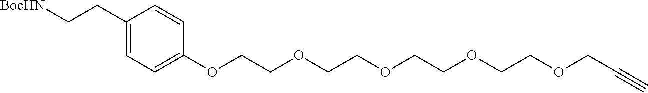 [SMILES: C#CCOCCOCCOCCOCCOc1ccc(CCNC(=O)OC(C)(C)C)cc1]

To a solution of boc-tyramine (0.45 g, 1.89 mmol) in acetonitrile (15 mL) was added cesium carbonate (1.23 g, 3.78 mmol) and the solution was stirred for 30 min Propargyl-Peg₄-mesylate (0.59 g, 1.89 mmol) was then added and the solution was heated to 70° C. and stirred for 16 hours. The excess acetonitrile was removed on the rotary evaporator and the crude material was purified by flash chromatography (0% to 100% EtOAc/hexanes). Yield 604 mg, 71%. Ion(s) found by LC/MS [M-Boc+H]+10=351.9.

### Step b

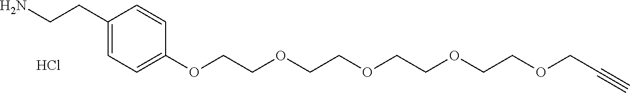 [SMILES: C#CCOCCOCCOCCOCCOc1ccc(CCN)cc1 ; Cl]

To Boc-amine from the previous step (604 mg, 1.33 mmol) was added 4N HCl in dioxane (6 mL). The solution was stirred for 2 hours at ambient temperature. The excess dioxane was removed and the crude material was used in the next reaction without additional purification. Ion LC/MS [M+H]+=351.8.

### Step c

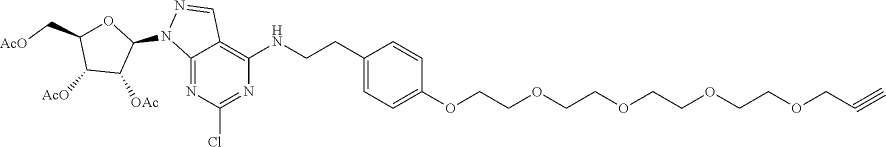 [SMILES: C#CCOCCOCCOCCOCCOc1ccc(CCNc2nc(Cl)nc3c2cnn3[C@@H]2O[C@H](COC(C)=O)[C@@H](OC(C)=O)[C@H]2OC(C)=O)cc1]

Intermediate B (600 mg, 1.33 mmol), the amine-HCl salt from the previous step (520 mg, 1.33 mmol) and DIEA (346 mg, 2.68 mmol) were stirred in ethanol (25 mL) at 50° C. for 4 hours. The mixture was cooled to ambient temperature and concentrated on the rotary evaporator. The crude mixture was purified by reversed phase flash chromatography (0% to 100% CH₃CN/H₂O). Yield 566 mg, 56%. Ion(s) found by LC/MS [M+H]+=761.5.

### Step d

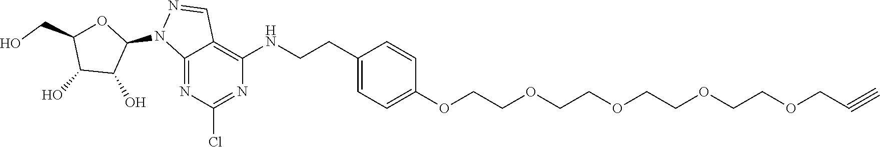 [SMILES: C#CCOCCOCCOCCOCCOc1ccc(CCNc2nc(Cl)nc3c2cnn3[C@@H]2O[C@H](CO)[C@@H](O)[C@H]2O)cc1]

To a solution of the triacetate from the previous step (566 mg, 0.74 mmol) in methanol (10 mL) was added sodium methoxide 25% in methanol (2 mL). The solution was stirred for 2 hours at ambient temperature. The crude mixture was neutralized with glacial acetic acid (3 mL) and the crude mixture was concentrated and purified by semi-preparative HPLC (0% to 100% CH₃CN/H₂O, 0.1% TFA modifier). Yield 376 mg. Ion(s) found by LC/MS [M+H]+=635.6.

### Step e

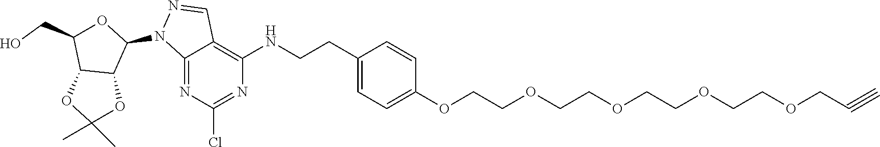 [SMILES: C#CCOCCOCCOCCOCCOc1ccc(CCNc2nc(Cl)nc3c2cnn3[C@@H]2O[C@H](CO)[C@H]3OC(C)(C)O[C@H]32)cc1]

To a solution of the triol from the previous step (376 mg, 0.59 mmol) in acetone (10 mL) was added 2,2-dimethoxypropane (1.69 g, 16.29 mmol) and TsOH (10 mg, 0.05 mmol). The solution was stirred for 16 h. The excess TsOH was neutralized with DIEA (1 mL) and the crude mixture was concentrated and purified by semi-preparative HPLC (0% to 100% CH₃CN/H₂O, 0.1% TFA modifier). Yield 273 mg, 67%. Ion(s) found by LC/MS [M+H]+=675.6.

### Step f

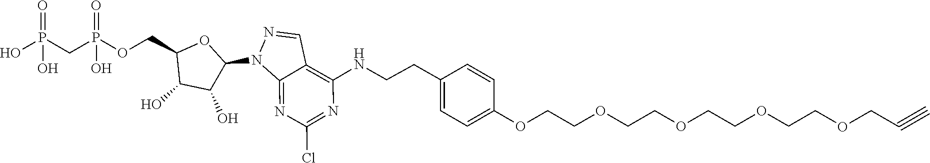 [SMILES: C#CCOCCOCCOCCOCCOc1ccc(CCNc2nc(Cl)nc3c2cnn3[C@@H]2O[C@H](COP(=O)(O)CP(=O)(O)O)[C@@H](O)[C@H]2O)cc1]

The acetonide from the previous step (273 mg, 0.40 mmol) in THE (6 mL) was added, dropwise, to a mixture of methylene (bis phosphonic dichloride) (302 mg, 1.21 mmol) and DIEA (77 mg, 0.60 mmol) in THE (10 mL) cooled to 0° C. via an ice bath. When the addition was complete the ice bath was removed and the reaction was stirred for 3 hours at ambient temperature. The mixture was then cooled to 0° C. via an ice bath and 0.1 N aqueous HCl (30 mL) was added. The mixture was stirred at 0° C. for 15 min then at ambient temperature for 16 hours (monitored by LC/MS). The solvent was removed on the rotary evaporator and the crude mixture was purified by semi-preparative HPLC (0% to 100% CH₃CN/H₂O, 0.1% TFA modifier). The pure fractions were pooled and lyophilized to afford the product as a white, hygroscopic solid. Yield 158 mg, 48%. Ion(s) found by LC/MS [M+H]+=794.2.

### Step g

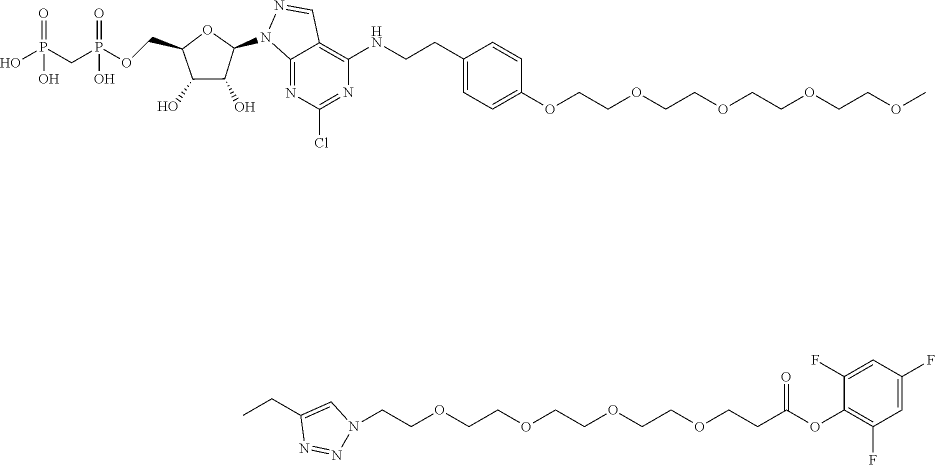

The alkyne from the previous step (158 mg, 0.20 mmol) and intermediate A (109 mg, 0.26 mmol), were dissolved in DMF (1 mL) and cooled to 0° C. via an ice water bath. Premixed copper(II) sulfate (12 mg, 0.01 mmol) and sodium ascorbate (40 mg, 0.20 mmol) in DI water (1 mL) were then added and the mixture was stirred for 4 hours at ambient temperature. The crude reaction mixture was purified directly via semi-preparative HPLC (0% to 100% CH₃CN/H₂O, 0.1% TFA modifier). The pure fractions were pooled and lyophilized to afford the product as a white, hygroscopic solid. Yield 30 mg, 13%. Ion(s) found by LC/MS [(M/2)+H]+=608.5.

### Synthesis of Conjugate 47

Trifluorophenol ester (described in the synthesis of Int-87) was conjugated to Fc carrier SEQ ID NO: 13 as described in the general conjugation procedure using bicarbonate buffer (0.1-0.3 mL) to adjust the pH to ˜8.5. Yield 89.4%, average MALDI mass=62121 Da. (DAR=3.7)

### Synthesis of Int-5

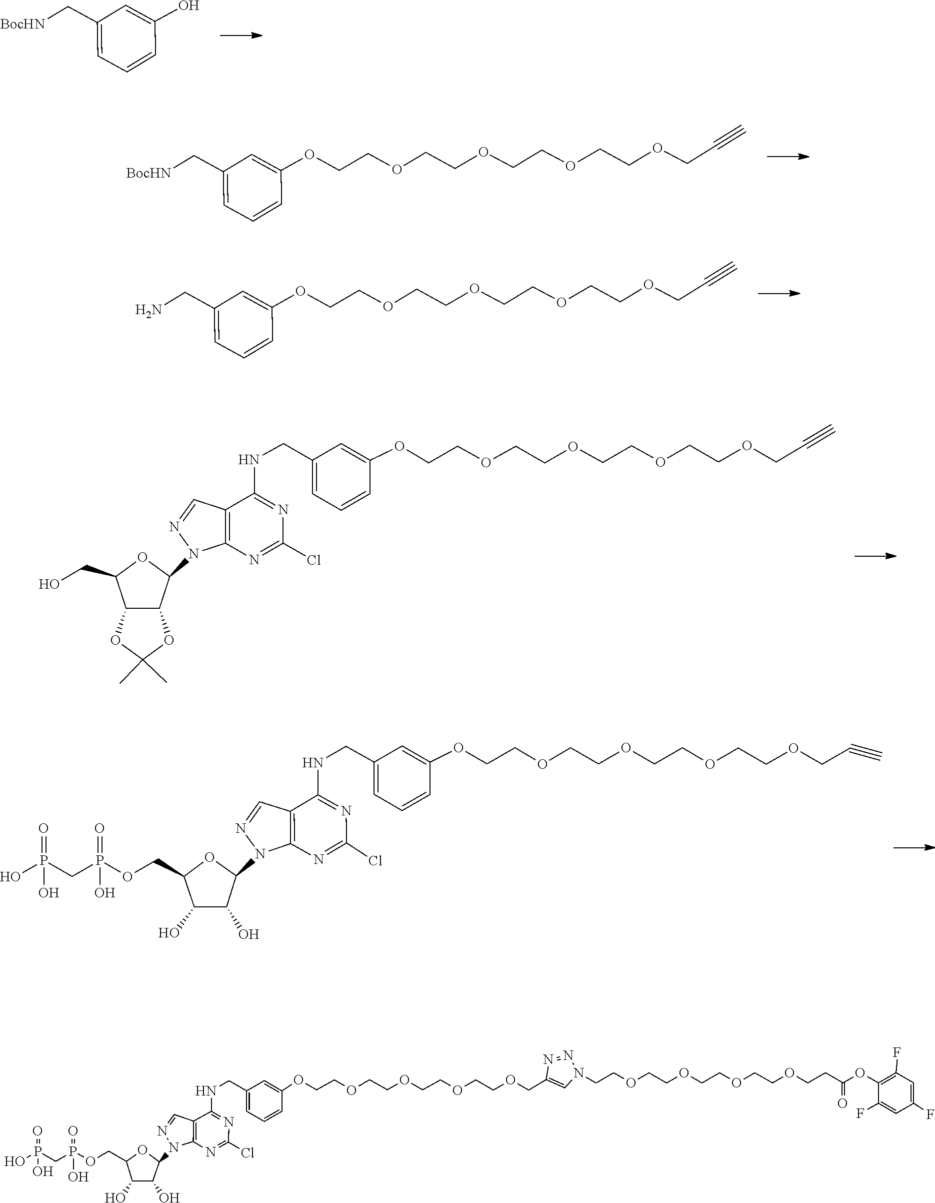 [SMILES: CC(C)(C)OC(=O)NCc1cccc(O)c1 ; C#CCOCCOCCOCCOCCOc1cccc(CNC(=O)OC(C)(C)C)c1 ; C#CCOCCOCCOCCOCCOc1cccc(CN)c1 ; C#CCOCCOCCOCCOCCOc1cccc(CNc2nc(Cl)nc3c2cnn3[C@@H]2O[C@H](CO)[C@H]3OC(C)(C)O[C@H]32)c1 ; C#CCOCCOCCOCCOCCOc1cccc(CNc2nc(Cl)nc3c2cnn3[C@@H]2O[C@H](COP(=O)(O)CP(=O)(O)O)[C@@H](O)[C@H]2O)c1 ; O=C(CCOCCOCCOCCOCCn1cc(COCCOCCOCCOCCOc2cccc(CNc3nc(Cl)nc4c3cnn4[C@@H]3OC(COP(=O)(O)CP(=O)(O)O)[C@@H](O)[C@H]3O)c2)nn1)Oc1c(F)cc(F)cc1F]

### Step a

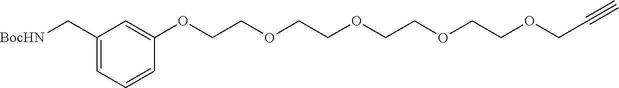 [SMILES: C#CCOCCOCCOCCOCCOc1cccc(CNC(=O)OC(C)(C)C)c1]

To a solution of tert-butyl 3-hydroxybenzylcarbamate (1.0 g, 4.47 mmol) and propargyl-PEG4 methanesulfonylate (1.4 g, 4.47 mmol) in acetonitrile (15 mL), was added potassium carbonate (1.23 g, 8.9 mmol) and cesium carbonate (0.15 g, 0.44 mmol). The reaction solution stirred at room temperature overnight, filtered, concentrated and purified by semi-preparative HPLC (5% to 95% acetonitrile and water, using 0.1% TFA as modifier). Yield 1.1 g, 60%. Ion(s) found by LCMS: [M+H]⁺=438.2.

### Step b

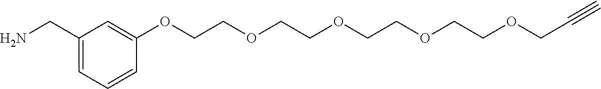 [SMILES: C#CCOCCOCCOCCOCCOc1cccc(CN)c1]

The product from the previous step (1.1 g, 2.6 mmol) was dissolved in a cool solution of HCl (10 ml, 4N in dioxane) and stirred for 2 hours at ambient temperature. The solution was concentrated and the crude product was carried to the subsequent step without purification. Yield of HCl salt 0.87 g. Ion(s) found by LCMS: [M+H]⁺=338.1

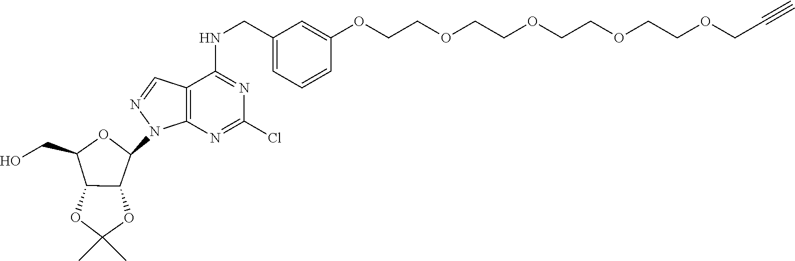 [SMILES: C#CCOCCOCCOCCOCCOc1cccc(CNc2nc(Cl)nc3c2cnn3[C@@H]2O[C@H](CO)[C@H]3OC(C)(C)O[C@H]32)c1]

A solution of the product from the previous step (0.87 g, 2.6 mmol), intermediate B (1.1 g, 2.4 mmol), and triethylamine (0.51 mL, 3.7 mmol) in anhydrous ethanol (5 mL) was sealed in a screw-top vial and heated to 70° C. overnight. After cooling to ambient temperature, 7 M ammonia in methanol (5 mL) was added and the mixture was stirred overnight. The reaction mixture was concentrated to dryness under reduced pressure, then dissolved in acetone (3.6 mL). 2,2-Dimethoxypropane (3.6 mL) and p-TsOH (0.6 g, 3.1 mmol) were added and the mixture was stirred overnight at ambient temperature. The reaction mixture was concentrated to dryness under reduced pressure and purified by silica gel chromatography (0→10% MeOH/DCM). Yield 0.98 g, 60%. Ion(s) found by LCMS: [M+H]⁺=662.2.

### Step d

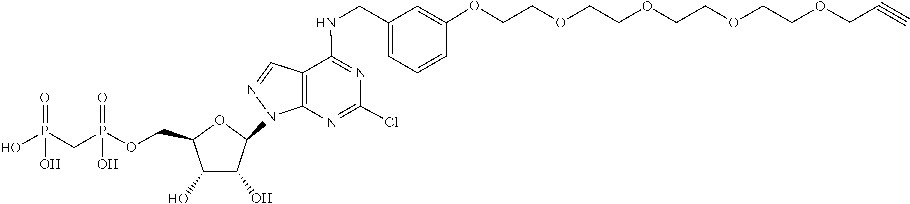 [SMILES: C#CCOCCOCCOCCOCCOc1cccc(CNc2nc(Cl)nc3c2cnn3[C@@H]2O[C@H](COP(=O)(O)CP(=O)(O)O)[C@@H](O)[C@H]2O)c1]

The product from the previous step (0.4 g, 0.6 mmol) in THE (2 mL) was added, dropwise, to a mixture of methylene (bis phosphonic dichloride) (0.56 g, 1.8 mmol) in THE (1 mL) cooled to 0° C. via an ice bath. When the addition was complete the ice bath was removed and the reaction was stirred for 3 hours at ambient temperature. The mixture was then cooled to 0° C. via an ice bath and 0.5 N aqueous HCl (6 mL) was added. The mixture was stirred at 0° C. for 15 minutes then at ambient temperature for 3 hours (monitored by LC/MS). The solvent was evaporated on the rotary evaporator and the crude mixture was purified by reversed phase HPLC (5-85% acetonitrile in DI water, 0.1% TFA modifier, 25 minute gradient). The pure fractions were pooled and lyophilized to afford the product as a white, hygroscopic solid. Yield 0.29 g, 55%. Ion(s) found by LCMS: [M+H]⁺=780.1.

### Step e

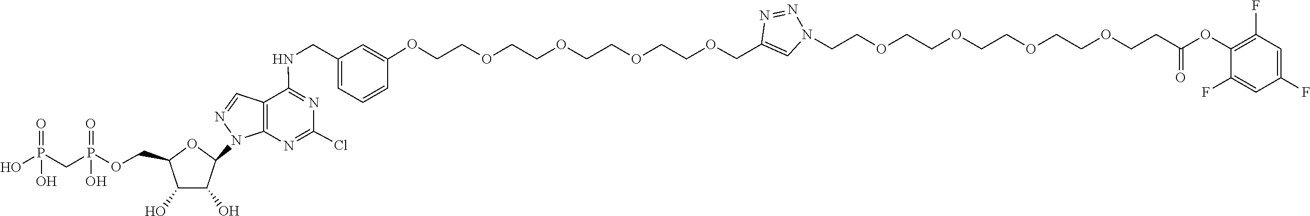 [SMILES: O=C(CCOCCOCCOCCOCCn1cc(COCCOCCOCCOCCOc2cccc(CNc3nc(Cl)nc4c3cnn4[C@@H]3O[C@H](COP(=O)(O)CP(=O)(O)O)[C@@H](O)[C@H]3O)c2)nn1)Oc1c(F)cc(F)cc1F]

The product from the previous step (80 mg, 0.1 mmol) and intermediate A (56 mg, 0.13 mmol) were dissolved in DMF (1 mL) and cooled to 0° C. via an ice water bath. Copper sulfate (4.1 mg, 0.02 mmol) was added to a mixture of THPTA (22 mg, 0.05 mmol) and sodium ascorbate (20 mg, 0.1 mmol) in DI water (2 mL) the mixture was gently shaken for 15-20 seconds until the solution was clear. The copper mixture was added to the alkyne/azido mixture and stirred at 0° C. for 10 minutes, then at ambient temperature for 20 minutes. The crude reaction mixture was purified directly by reversed phase HPLC (5-85% acetonitrile in DI water, 0.1% TFA modifier, 25 minute gradient). The pure fractions were pooled and lyophilized to afford the product as a white, hygroscopic solid. Yield 86.8 mg, 70%. Ion(s) found by LCMS: [M+2H)/2]⁺=601.1.

### Synthesis of Conjugate 145

To a solution of Fc SEQ ID NO: 13 (7.07 ml, 137 mg, 0.002497 mmol) in PBS 7.4 was added active ester described in Int-5 (30 mg, 0.02497 mmol) in DMF (1.75 ml). The pH of the reaction mixture was slowly adjusted to ˜8.5 with 1M borate buffer solution (0.8 ml). The reaction was then gently rotate overnight and then purified according to the general procedure. Maldi TOF analysis of the purified final product gave an average mass of 63,384 Da (DAR=4.9). Yield: 108.3 mg, 80.2%.

### Synthesis of Int-8

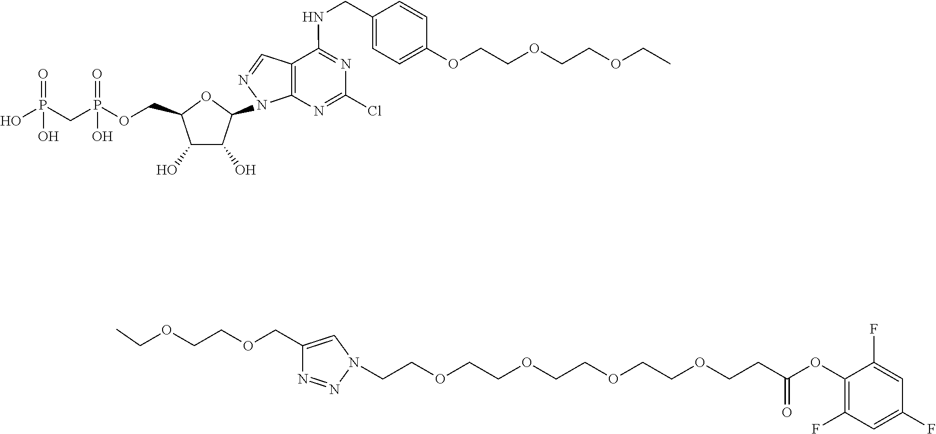

The title compound was prepared analogously to Int-5, where tert-butyl 3 hydroxybenzyl carbamate was replaced with tert-butyl 4-hydroxybenzylcarbamate. Ions found by LCMS: [M+2H)/2]⁺=601.3.

### Synthesis of Conjugate 6

The title compound was prepared analogously to Conjugate 145, where the product described in Int-5 was replaced with Int-8. Maldi TOF analysis of the purified final product gave an average mass of 62237 Da (DAR=3.8). Yield: 116.2 mg, 77.5%.

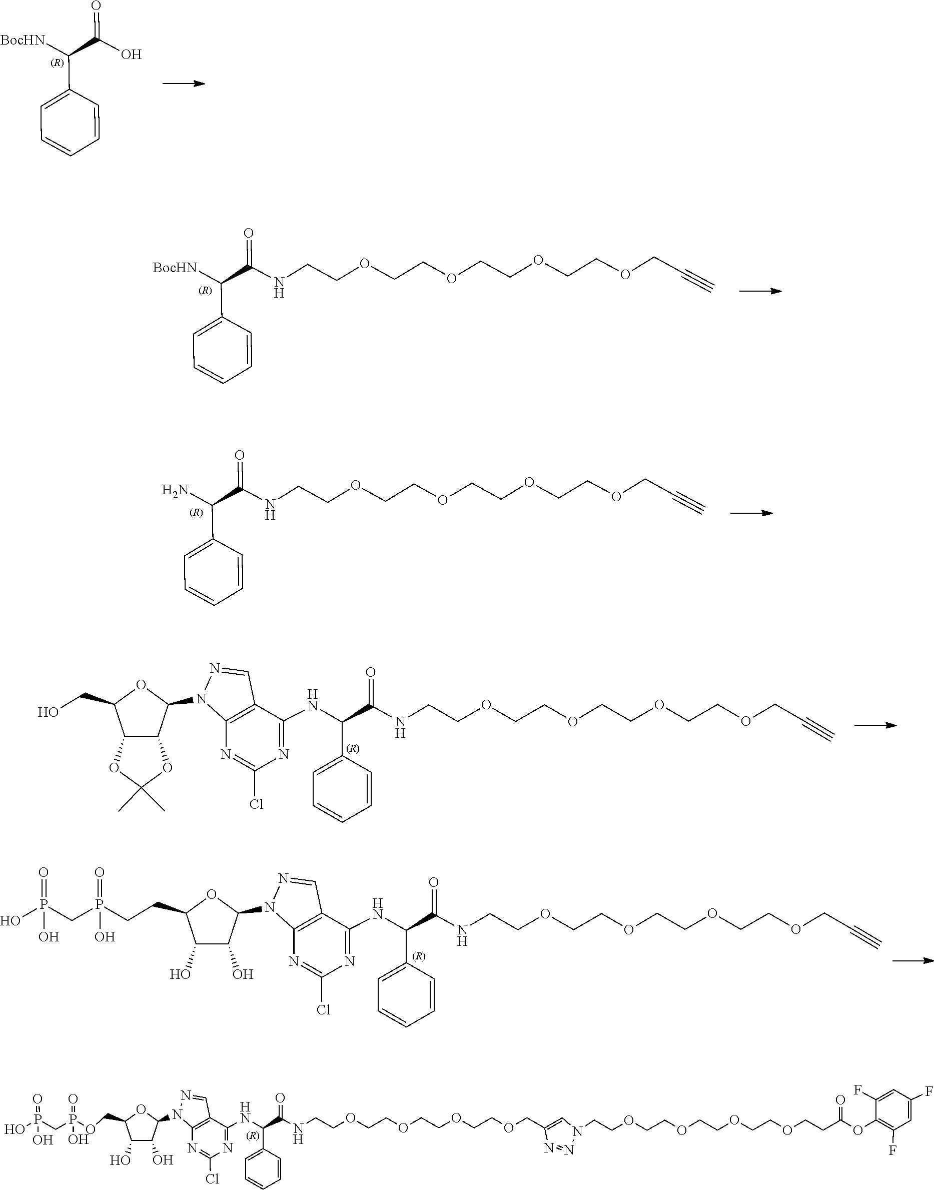

### Step a

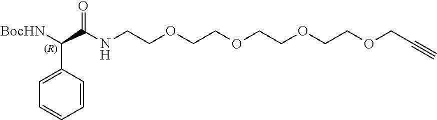

To a solution of (R)-2-((tert-butoxycarbonyl)amino)-2-phenylacetic acid (0.5 g, 2 mmol) and propargyl-peg4-amine (0.46 g, 2 mmol) in DMF (10 ml) was added HATU (1.5 g, 3.9 mmol) and DIEA (0.6 ml, 3.9 mmol). The reaction solution was stirred at room temperature overnight, concentrated and purified by reversed phase HPLC (5% to 95% acetonitrile and water, using 0.1% TFA as modifier). Yield 0.54 g, 58%. Ion(s) found by LCMS: [M+H]⁺=465.2.

### Step b

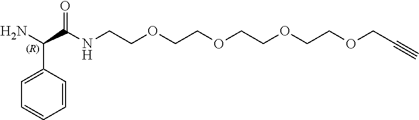

The product from the previous step was dissolved in a solution of HCl (5 ml, 4N in dioxane), pre-cooled to 0° C. via an ice/water bath and reaction was stirred for 2 hours. The solution was concentrated and the crude product was carried to the subsequent step without purification. Ion(s) found by LCMS: [M+H]⁺=365.2.

### Step c

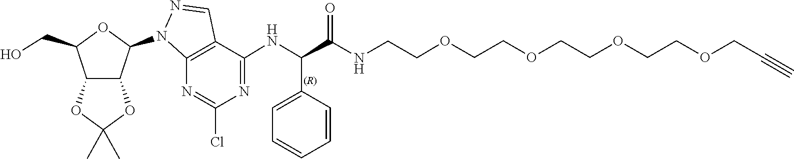

The product from the previous step (0.41 g, 1.0 mmol), intermediate B (0.49 g, 1.1 mmol), and triethylamine (0.23 mL, 1.66 mmol) were dissolved in anhydrous ethanol (2 mL) and heated in a sealed tube at 70° C. overnight. After cooling to ambient temperature, 7 M ammonia in methanol (2 mL) was added and the mixture was stirred overnight at ambient temperature. The reaction mixture was concentrated to dryness under reduced pressure, then dissolved in acetone (1.6 mL). 2,2-dimethoxypropane (1.6 mL) and p-TsOH (0.26 g, 1.4 mmol) were added and the mixture was stirred overnight at ambient temperature. The reaction mixture was concentrated to dryness under reduced pressure and purified by silica gel chromatography (0→10% MeOH/DCM). Yield 0.51 g, 66%. Ion(s) found by LCMS: [M+H]⁺=689.1.

### Step d

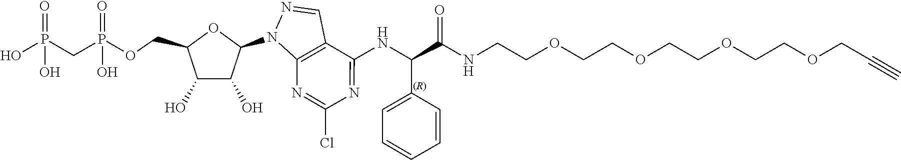

The product from the previous step (375 mg, 0.54 mmol) in THE (2 mL) was added, dropwise, to a mixture of methylene (bis phosphonic dichloride) (510 mg, 1.63 mmol) and DIEA (0.1 ml, 0.6 mmol) in THE (1 mL) cooled to 0° C. via an ice bath. When the addition was complete the ice bath was removed and the reaction was stirred for 3 hours at ambient temperature. The mixture was then cooled to 0° C. via an ice bath and 0.5 N aqueous HCl (1 mL) was added. The mixture was stirred at 0° C. for 15 minutes then at ambient temperature for 3 hours (monitored by LC/MS). The solvent was reduced to ˜2 mL on the rotary evaporator and the crude mixture was purified by reversed phase HPLC (5-85% acetonitrile in DI water, 0.1% TFA modifier, 25 minute gradient). The pure fractions were pooled and lyophilized to afford the product as a white, hygroscopic solid. Yield 280 mg, 63.7%. Ion(s) found by LCMS: [M+H]⁺=805.2.

### Step e

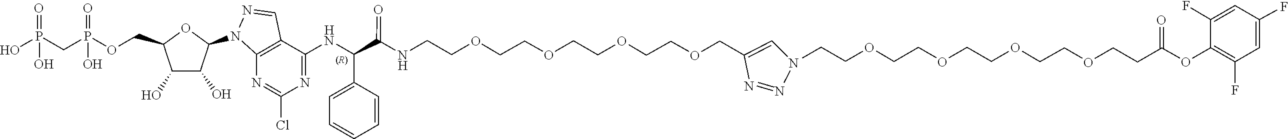

The product from the previous step (80 mg, 0.1 mmol) and intermediate A (54 mg, 0.12 mmol) were dissolved in DMF (0.6 mL) and cooled to 0° C. via an ice water bath. Copper sulfate (3.1 mg, 0.019 mmol) was added to a mixture of THPTA (21.5 mg, 0.05 mmol) and sodium ascorbate (19.6 mg, 0.1 mmol) in DI water (0.6 mL) the mixture was gently shaken for 15-20 seconds until the solution was clear. The copper mixture was added to the alkyne/azido mixture and the reaction was stirred at 0° C. for 10 minutes then at ambient temperature for 30 minutes. The crude reaction mixture was purified directly by reversed phase HPLC (5-85% acetonitrile in DI water, 0.1% TFA modifier, 25 minute gradient). The pure fractions were pooled and lyophilized to afford the product as a white, hygroscopic solid. Yield 88 mg, 72%. Ions found by LCMS: [M+2H)/2]⁺=614.5.

### Synthesis of Conjugate 12

To a solution of Fc SEQ ID NO: 13 (4.15 ml, 80.9 mg, 0.00146 mmol) in PBS 7.4 was added Int-18 (18 mg, 0.0146 mmol) in DMF (1 ml). The pH of the reaction mixture was slowly adjusted to ˜8.5 with 1M borate buffer solution (0.6 ml). The reaction was then gently rotated overnight and then purified according to the general procedure. Maldi TOF analysis of the purified final product gave an average mass of 63,437 Da (DAR=4.8). Yield: 61.3 mg, 76.7%.

### Synthesis of Int-51

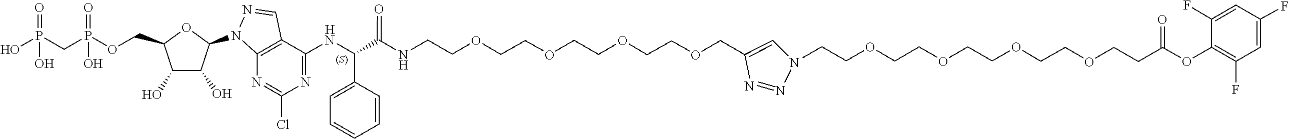

The title compound was prepared analogously to Int-18, where (R)-2-((tert-butoxycarbonyl) amino)-2-phenylacetic acid was replaced with (S)-2-((tert-butoxycarbonyl)amino)-2-phenylacetic acid. Ions found by LCMS: [M+2H)/2]⁺=614.9.

### Synthesis of Conjugate 29

The title compound was prepared using general conjugation procedure and the product described in Int-51. Maldi TOF analysis of the purified final product gave an average mass of 64,484 Da (DAR=5.6). Yield: 77.4 mg, 85.9%.

### Synthesis of Int-108

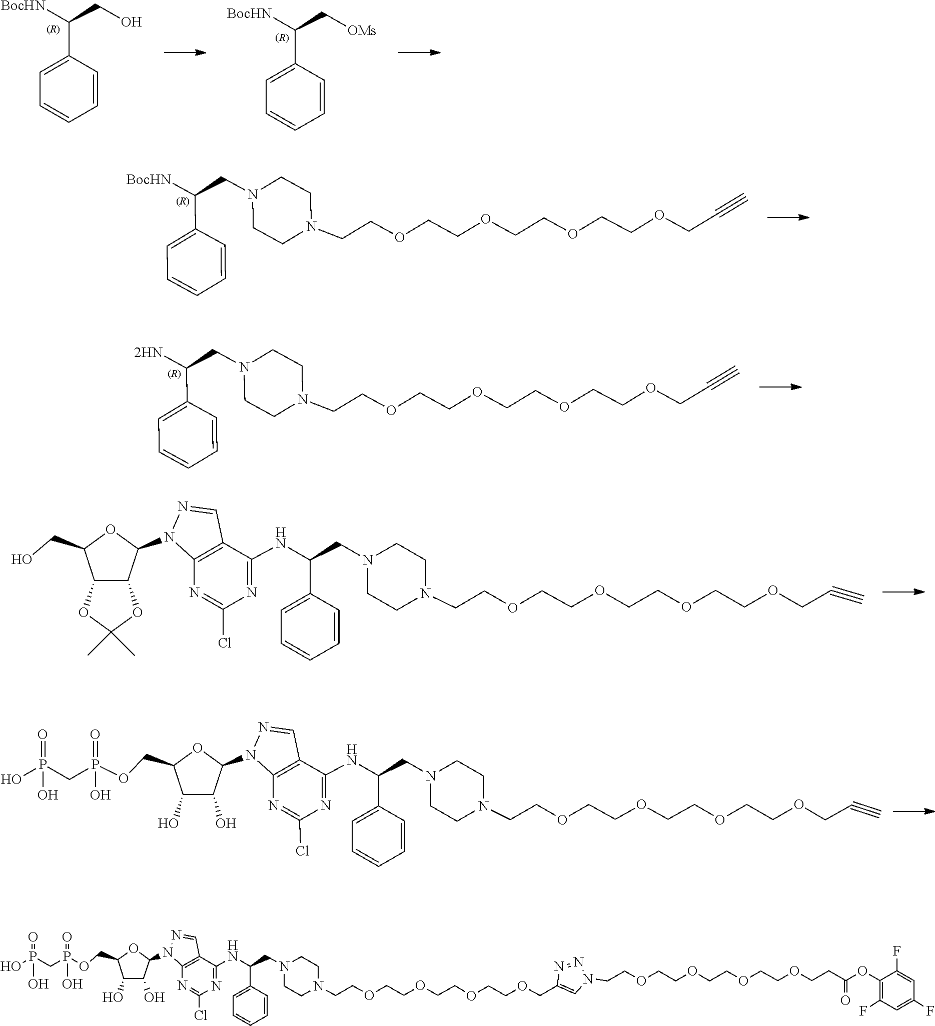

### Step a

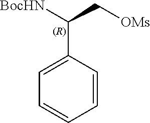

To a solution of (R)-tert-butyl (2-hydroxy-1-phenylethyl)carbamate (2 g, 8.4 mmol) in DCM (40 ml) was added methanesulfonyl chloride (1 ml, 12.6 mmol), and triethylamine (2.34 ml, 16.8 mmol). The reaction was stirred for 2 hours at ambient temperature. The reaction was diluted with ethyl acetate (40 mL) washed with aqueous 1 N HCl, DI water, and brine. The combined organic extracts were dried over sodium sulfate and concentrated under reduced pressure to afford the product. Yield 2.2 g, 82%. Ions found by LCMS [M+H]+=316.3

### Step b

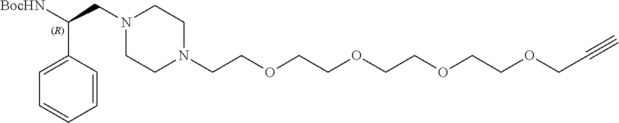

To the product from the previous step (0.35 g, 1.11 mmol) and piperazine-peg4-propargyl (0.40 g, 1.11 mmol) in acetonitrile (4 mL), was added potassium carbonate (0.46 g, 3.33 mmol). The reaction was stirred at 60° C. overnight, filtered and concentrated. The product was purified by semi-preparative HPLC (5% to 95% acetonitrile and water, using 0.1% TFA as modifier). Yield 0.36 mg, 62%. Ion(s) found by LCMS: [M+H]⁺=520.3.

### Step c

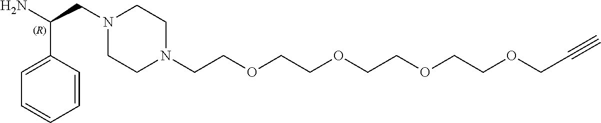

The product from the previous step was dissolved in a cool solution of 4N HCl in dioxane (4 mL) and stirred for 2 hours at ambient temperature. The solution was concentrated and the product was carried to the next step without purification. Ion(s) found by LCMS: [M+H]⁺=420.2.

### Step d

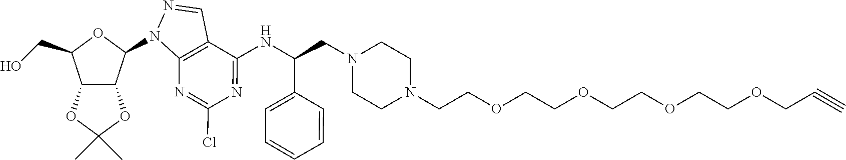 [SMILES: C#CCOCCOCCOCCOCCN1CCN(C[C@H](Nc2nc(Cl)nc3c2cnn3[C@@H]2O[C@H](CO)[C@H]3OC(C)(C)O[C@H]32)c2ccccc2)CC1]

The product from the previous step (78 mg, 0.187 mmol), intermediate B (122 mg, 0.178 mmol), and triethylamine (0.037 mL, 0.267 mmol) in anhydrous ethanol (1 mL) were heated in a screw-top vial at 70° C. overnight. After cooling to room temperature, 7 M ammonia in methanol (1 mL) was added and the mixture was stirred overnight at ambient temperature. The reaction mixture was concentrated to dryness under reduced pressure, then dissolved in acetone (0.26 mL). 2,2-Dimethoxypropane (0.26 mL) and p-TsOH (42 mg, 0.22 mmol) were added and the mixture was stirred overnight at ambient temperature. The reaction mixture was concentrated to dryness under reduced pressure and purified by silica gel chromatography (0→10% MeOH in DCM). Yield 42 mg, 32%. Ion(s) found by LCMS: [M+H]⁺=744.3.

### Step e

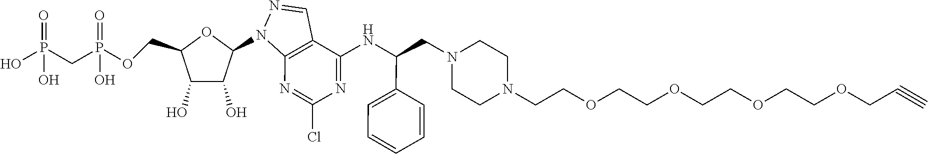 [SMILES: C#CCOCCOCCOCCOCCN1CCN(C[C@H](Nc2nc(Cl)nc3c2cnn3[C@@H]2O[C@H](COP(=O)(O)CP(=O)(O)O)[C@@H](O)[C@H]2O)c2ccccc2)CC1]

The product from the previous step (42 mg, 0.056 mmol) in THE (0.2 mL) was added, dropwise, to a mixture of methylene (bis phosphonic dichloride) (52 mg, 0.17 mmol) and DIEA (0.01 ml, 0.06 mmol) in THE (0.1 mL) cooled to 0° C. via an ice bath. When the addition was complete the ice bath was removed and the reaction was stirred for 3 hours at ambient temperature. The mixture was then cooled to 0° C. via an ice bath and 0.5 N aqueous HCl (1 mL) was added. The mixture was stirred at 0 C for 15 minutes then at ambient temperature for 3 hours (monitored by LC/MS). The solvent was reduced to ˜2 mL on the rotary evaporator and the crude mixture was purified by reversed phase HPLC (5-85% acetonitrile in DI water, 0.1% TFA modifier, 25 minute gradient). The pure fractions were pooled and lyophilized to afford the product as a white, hygroscopic solid. Yield 24 mg, 50%. Ion(s) found by LCMS: [M+H]⁺=862.2.

### Step f

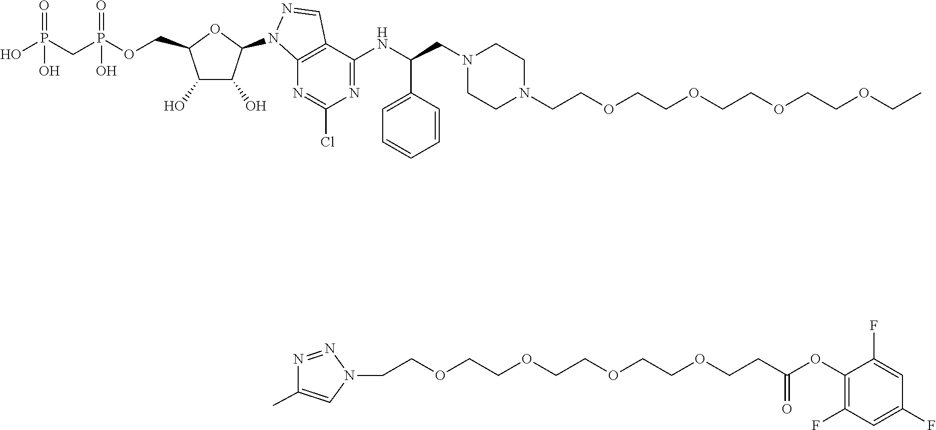

The product from the previous step (24 mg, 0.027 mmol) and intermediate A (15 mg, 0.036 mmol), were dissolved in DMF (0.6 mL) and cooled to 0° C. via an ice water bath. Copper sulfate (1 mg, 0.005 mmol) was added to a mixture of THPTA (6 mg, 0.014 mmol) and sodium ascorbate (5.5 mg, 0.027 mmol) in DI water (0.6 mL) the mixture was gently shaken for 15-20 seconds until the solution was clear.

The copper mixture was added to the alkyne/azido mixture and the reaction was stirred at 0° C. for 10 minutes then at ambient temperature for 20 minutes. The reaction mixture was purified directly by reversed phase HPLC (5-85% acetonitrile in DI water, 0.1% TFA modifier, 25 minute gradient). The pure fractions were pooled and lyophilized to afford the product as a white, hygroscopic solid. Yield 21 mg, 58%. Ion(s) found by LC/MS [(M+2H/2)]+=642.5.

### Synthesis of Conjugate 57

To a solution of Fc SEQ ID NO: 13 (1.53 ml, 30 mg, 0.00054 mmol) in PBS 7.4 was added Int-108 (7 mg, 0.0054 mmol) in DMF (0.4 ml). The pH of the reaction mixture was slowly adjusted to ˜8.5 with 1M borate buffer solution (0.2 ml). The reaction was then gently rotated overnight and then purified according to the general procedure. Maldi TOF analysis of the purified final product gave an average mass of 64,521 Da (DAR=5.8). Yield: 44.6 mg, 100%.

### Synthesis of Int-95

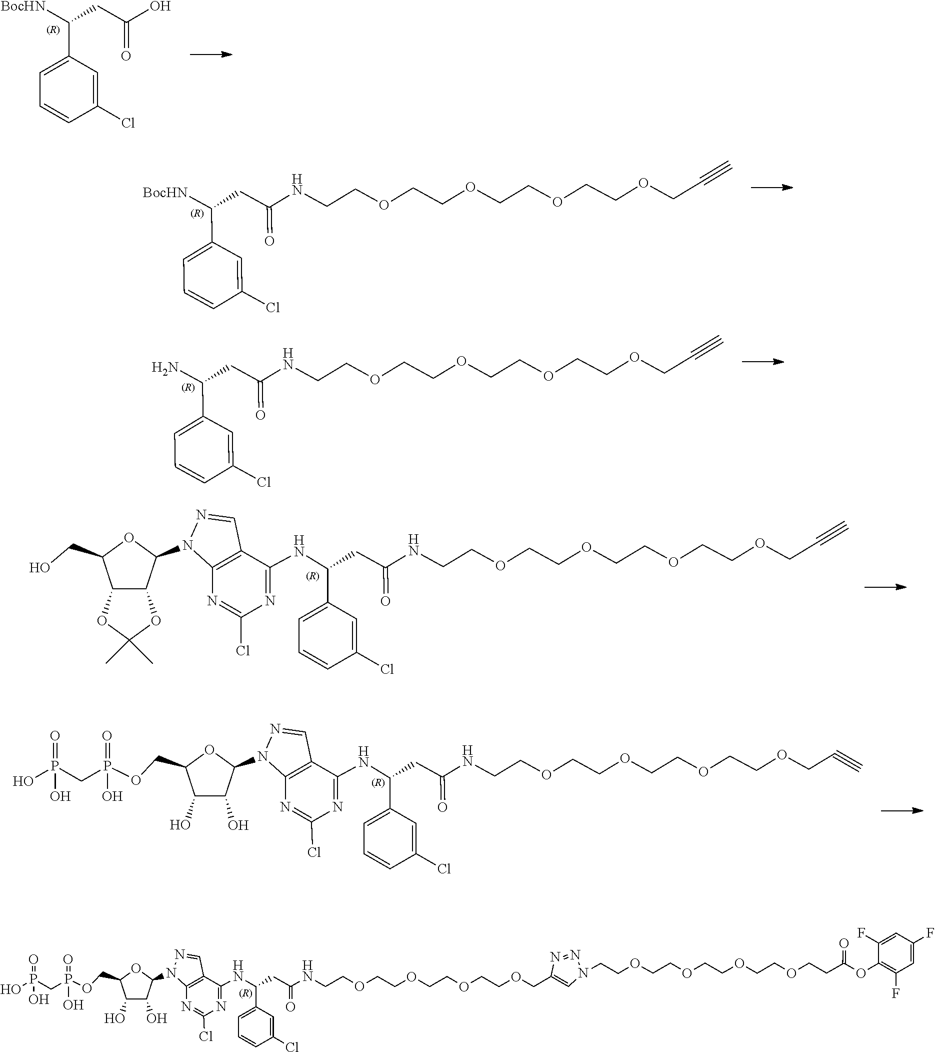

### Step a

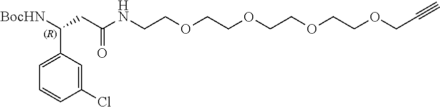

To a mixture of (R)-3-((tert-butoxycarbonyl)amino)-3-(3-chlorophenyl)propanoic acid (0.36 g, 1.2 mmol), and propargyl-Peg 4-amine (0.23 g, 1 mmol) in DMF (5 ML) was added HATU (0.76 g, 2 mmol) and DIPEA (0.35 ml, 2 mmol). The reaction was stirred at room temperature overnight, concentrated and purified by RPLC. Yield 0.35 g, 70%. Ion(s) found by LCMS: [M+H]⁺=513.0.

### Step b

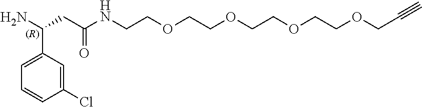

The product from the previous step was dissolved in a 4N HCl in dioxane (5 mL) cooled to 0° C. via an ice water bath and reaction was stirred for 2 hours. The solution was concentrated and the residue was used as an intermediate without purification. Ion(s) found by LC/MS [M+H]+=413.1.

### Step c

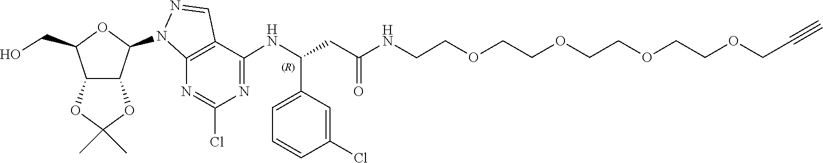

The product from the previous step (0.32 g, 0.71 mmol), intermediate B (0.42 g, 0.75 mmol), and triethylamine (0.15 mL, 1.1 mmol) were dissolved in anhydrous ethanol (2 mL) and heated in a sealed tube at 70° C. overnight. After cooling to ambient temperature, 7 M ammonia in methanol (2 mL) was added and the mixture was stirred overnight at ambient temperature. The reaction mixture was concentrated to dryness under reduced pressure, then dissolved in acetone (1.0 mL). 2,2-dimethoxypropane (1.0 mL) and p-TsOH (0.17 g, 0.9 mmol) were added and the mixture was stirred overnight at ambient temperature. The reaction mixture was concentrated to dryness under reduced pressure and purified by silica gel chromatography (0→10% MeOH/DCM). Yield 0.31 g, 65%. Ion(s) found by LCMS: [M+H]⁺=737.6.

### Step d

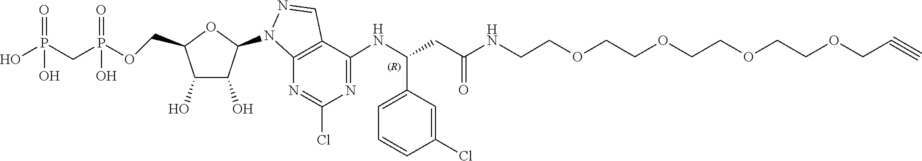

The acetonide from the previous step (315 mg, 0.42 mmol) in THE (1.2 mL) was added, dropwise, to a mixture of methylene (bis phosphonic dichloride) (330 mg, 1.28 mmol) and DIEA (0.09 mL, 0.05 mmol) in THE (0.6 mL) cooled to 0° C. via an ice bath. When the addition was complete the ice bath was removed and the reaction was stirred for 3 hours at ambient temperature. The mixture was then cooled to 0° C. via an ice bath and 0.5 N aqueous HCl (5 mL) was added. The mixture was stirred at 0° C. for 15 minutes then at ambient temperature for 3 hours (monitored by LC/MS). The solvent was reduced to ˜2 mL on the rotary evaporator, then the crude mixture was purified by reversed phase HPLC (5-85% acetonitrile in DI water, 0.1% TFA modifier, 25 minute gradient). The pure fractions were pooled and lyophilized to afford the product as a white, hygroscopic solid. Yield 68%, 250 mg. Ion(s) found by LCMS: [M+H]⁺=855.4.

### Step e

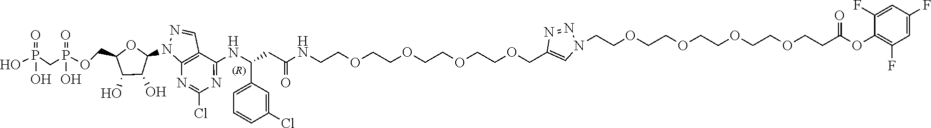

The product from the previous step (80 mg, 0.093 mmol) and intermediate A (51 mg, 0.12 mmol), were dissolved in DMF (0.6 mL) and cooled to 0° C. via an ice water bath. Copper sulfate (2.9 mg, 0.018 mmol) was added to a mixture of THPTA (20 mg, 0.046 mmol) and sodium ascorbate (18 mg, 0.092 mmol) in DI water (0.6 mL) the mixture was gently shaken for 15-20 seconds until the solution was clear. The copper mixture was added to the alkyne/azido mixture and the reaction was stirred at 0° C. for 10 minutes then at ambient temperature for 20 minutes. 125 mM sodium ascorbate solution (˜1 mL, pH 6) was added and the reaction mixture was purified directly by reversed phase HPLC (5-85% acetonitrile in DI water, 0.1% TFA modifier, 25 minute gradient). The pure fractions were pooled and lyophilized to afford the product as a white, hygroscopic solid. Yield 45 mg, 43%. Ions found by LCMS: [M+2H)/2]⁺=639.4.

### Synthesis of Conjugate 51

To a solution of Fc SEQ ID NO: 13 (5.7 ml, 111 mg, 0.00201 mmol) in PBS 7.4 was added Int-95 (28 mg, 0.0201 mmol) in DMF (1.5 ml). The pH of the reaction mixture was slowly adjusted to ˜8.5 with 1M borate buffer solution (0.2 ml). The reaction was then gently rotated overnight and then purified according to the general procedure. Maldi TOF analysis of the purified final product gave an average mass of 64,496 Da (DAR=5.6). Yield: 93 mg, 84.6%.

### Synthesis of Int-53

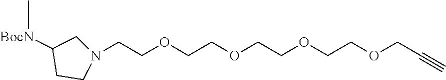 [SMILES: C#CCOCCOCCOCCOCCN1CCC(N(C)C(=O)OC(C)(C)C)C1]

### Step a

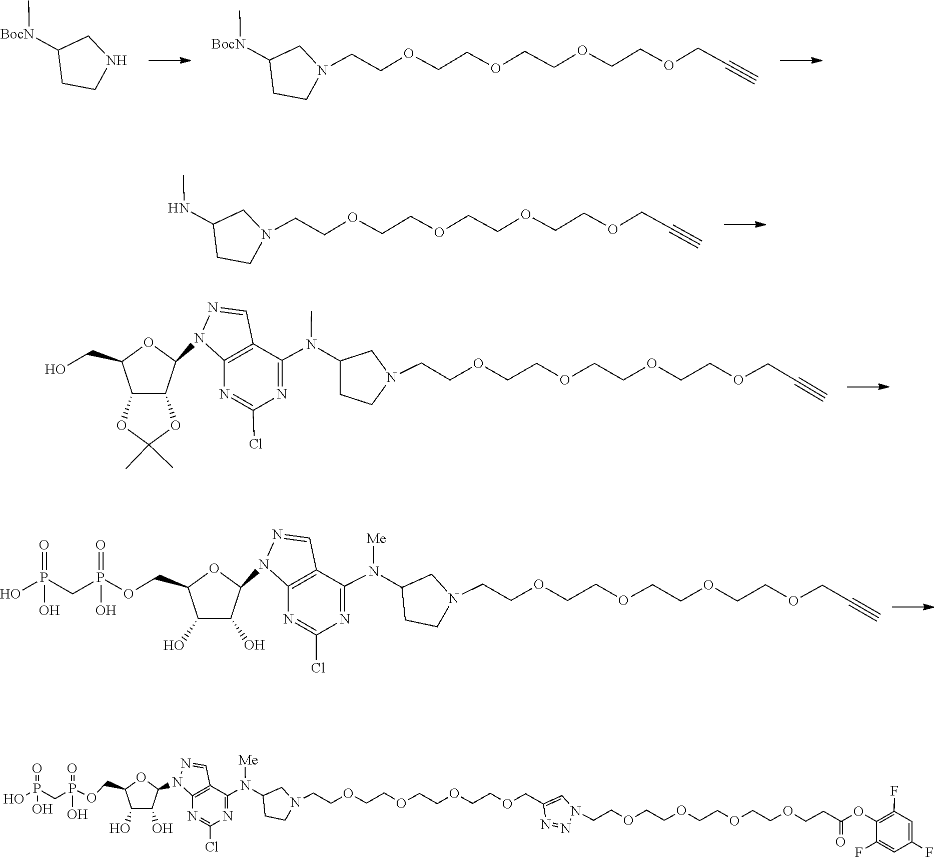 [SMILES: CN(C(=O)OC(C)(C)C)C1CCNC1 ; C#CCOCCOCCOCCOCCN1CCC(N(C)C(=O)OC(C)(C)C)C1 ; C#CCOCCOCCOCCOCCN1CCC(NC)C1 ; C#CCOCCOCCOCCOCCN1CCC(N(C)c2nc(Cl)nc3c2cnn3[C@@H]2O[C@H](CO)[C@H]3OC(C)(C)O[C@H]32)C1 ; C#CCOCCOCCOCCOCCN1CCC(N(C)c2nc(Cl)nc3c2cnn3[C@@H]2O[C@H](COP(=O)(O)CP(=O)(O)O)[C@@H](O)[C@H]2O)C1 ; CN(c1nc(Cl)nc2c1cnn2[C@@H]1O[C@H](COP(=O)(O)CP(=O)(O)O)[C@@H](O)[C@H]1O)C1CCN(CCOCCOCCOCCOCc2cn(CCOCCOCCOCCOCCC(=O)Oc3c(F)cc(F)cc3F)nn2)C1]

To a solution of tert-butyl methyl(pyrrolidin-3-yl)carbamate (220 mg, 1.1 mmol) and propargyl-Peg₄-methanesulfonylate (375 mg, 1.2 mmol) in acetonitrile (1 mL), was added potassium carbonate (456 mg, 3.3 mmol). The reaction solution stirred at 60° C. overnight. The mixture was filtered and concentrated. The crude residue was purified by semi-preparative HPLC (5% to 95% acetonitrile and water, using 0.1% TFA as modifier). Yield 250 mg, 54%. Ion(s) found by LCMS: [M+H]⁺=415.5

### Step b

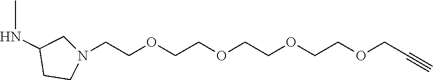 [SMILES: C#CCOCCOCCOCCOCCN1CCC(NC)C1]

The product from the previous step was dissolved in a solution of 4 N HCl in dioxane (5 ml) cooled to 0 C via an ice water bath, then the reaction was stirred for 2 hours at ambient temperature. The solution was concentrated and the residue was used without purification. LC/MS [M+H]+=315.4.

### Step c

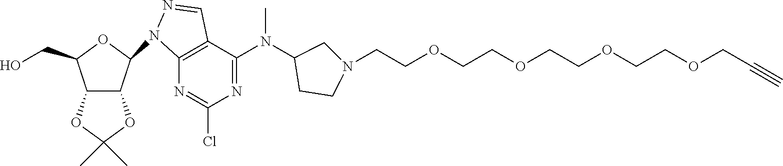 [SMILES: C#CCOCCOCCOCCOCCN1CCC(N(C)c2nc(Cl)nc3c2cnn3[C@@H]2O[C@H](CO)[C@H]3OC(C)(C)O[C@H]32)C1]

The product from the previous step (250 mg, 0.57 mmol), intermediate B (180 mg, 0.6 mmol), and triethylamine (0.12 mL, 0.86 mmol) in anhydrous ethanol (2 mL) were heated in a screw-top vial at 70° C. overnight. After cooling to room temperature, 7 M ammonia in methanol (2 mL) was added and the mixture was stirred overnight at ambient temperature. The reaction mixture was concentrated to dryness under reduced pressure, then dissolved in acetone (0.8 mL). 2,2-Dimethoxypropane (0.8 mL) and p-TsOH (130 mg, 0.72 mmol) were added and the mixture was stirred overnight at ambient temperature. The reaction mixture was concentrated to dryness under reduced pressure and purified by silica gel chromatography (0 →10% MeOH in DCM). Yield 152 mg, 41%. Ion(s) found by LCMS: [M+H]⁺=640.1.

### Step d

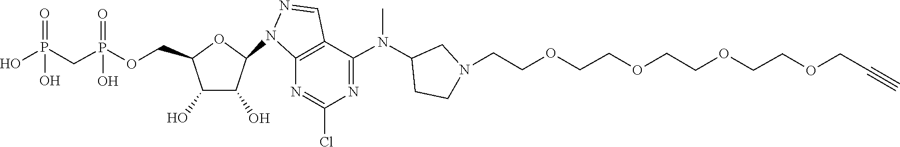 [SMILES: C#CCOCCOCCOCCOCCN1CCC(N(C)c2nc(Cl)nc3c2cnn3[C@@H]2O[C@H](COP(=O)(O)CP(=O)(O)O)[C@@H](O)[C@H]2O)C1]

The acetonide (152 mg, 0.24 mmol) in THE (0.8 mL) was added dropwise to a mixture of methylene (bis phosphonic dichloride) (220 mg, 0.71 mmol) and DIEA (0.045 mL, 0.26 mmol) in THE (0.4 mL) cooled to 0° C. via an ice bath. When the addition was complete the ice bath was removed and the reaction was stirred for 3 hours at ambient temperature. The mixture was then cooled to 0° C. via an ice bath and 0.5 N aqueous HCl (2 mL) was added. The mixture was stirred at 0° C. for 15 minutes then at ambient temperature for 3 hours (monitored by LC/MS). The solvent was reduced to ˜2 mL on the rotary evaporator and the crude mixture was purified by reversed phase HPLC (5-85% acetonitrile in DI water, 0.1% TFA modifier, 25 minute gradient). The pure fractions were pooled and lyophilized to afford the product as a white, hygroscopic solid. Yield 121 mg, 67%. LC/MS [M+H]+=757.0.

### Step e

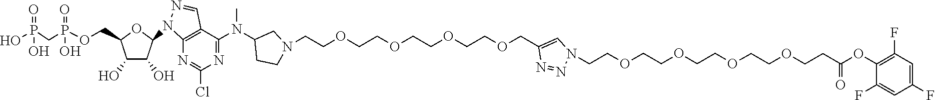 [SMILES: CN(c1nc(Cl)nc2c1cnn2[C@@H]1O[C@H](COP(=O)(O)CP(=O)(O)O)[C@@H](O)[C@H]1O)C1CCN(CCOCCOCCOCCOCc2cn(CCOCCOCCOCCOCCC(=O)Oc3c(F)cc(F)cc3F)nn2)C1]

The product from the previous step (61 mg, 0.08 mmol) and intermediate A (44 mg, 0.1 mmol), were dissolved in DMF (1 mL) and cooled to 0 C via an ice water bath. Copper sulfate (2.5 mg, 0.016 mmol) was added to a mixture of THPTA (17.5 mg, 0.04 mmol) and sodium ascorbate (16 mg, 0.08 mmol) in DI water (1 mL) the mixture was gently shaken for 15-20 seconds until the solution was clear.

The copper mixture was added to the alkyne/azido mixture and the reaction was stirred at 0 C for 10 minutes then at ambient temperature for 20 minutes. The reaction mixture was purified directly by reversed phase HPLC (5-85% acetonitrile in DI water, 0.1% TFA modifier, 25 minute gradient). The pure fractions were pooled and lyophilized to afford the product as a white, hygroscopic solid. Yield 65%, 62 mg. Ions found by LCMS: [M+2H)/2]⁺=590.2.

### Synthesis of Conjugate 30

To a solution of Fc SEQ ID NO: 13 (4.8 ml, 93.7 mg, 0.00169 mmol) in PBS 7.4 was added the product of Int-53 (20 mg, 0.0169 mmol) in DMF (0.255 ml). The pH of the reaction mixture was slowly adjusted to ˜8.5 with 1M borate buffer solution (0.2 ml). The reaction was then gently rotated overnight and then purified according to the general procedure. Maldi TOF analysis of the purified final product gave an average mass of 63,294 Da (DAR=5.8). Yield: 88.1 mg, 93.7%.

### Synthesis of Int-39

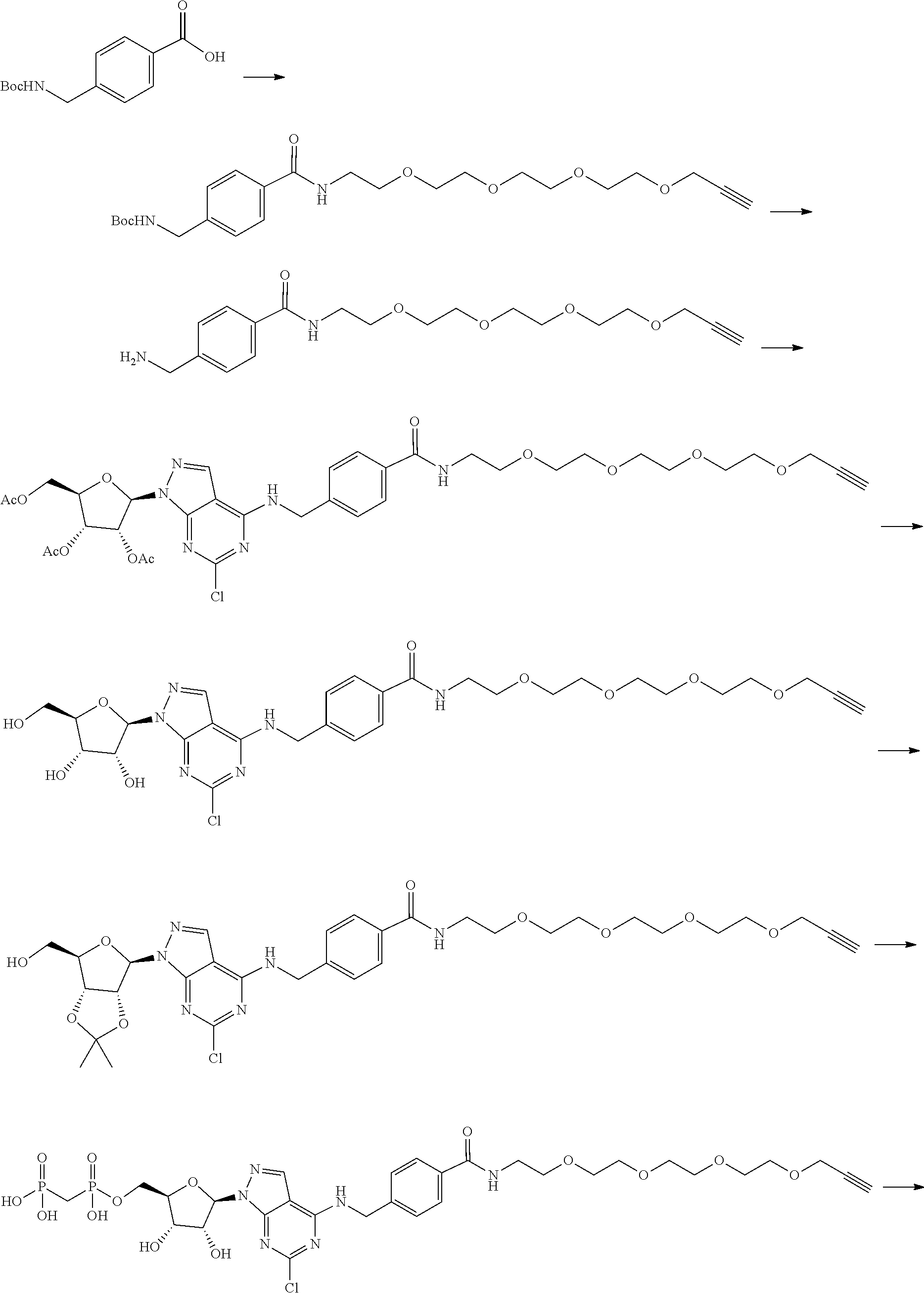

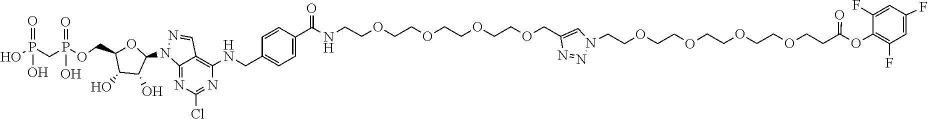 [SMILES: O=C(CCOCCOCCOCCOCCn1cc(COCCOCCOCCOCCNC(=O)c2ccc(CNc3nc(Cl)nc4c3cnn4[C@@H]3O[C@H](COP(=O)(O)CP(=O)(O)O)[C@@H](O)[C@H]3O)cc2)nn1)Oc1c(F)cc(F)cc1F]

### Step a

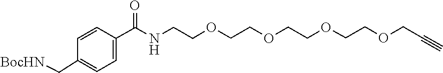 [SMILES: C#CCOCCOCCOCCOCCNC(=O)c1ccc(CNC(=O)OC(C)(C)C)cc1]

To a solution of 4-(Boc-aminomethyl)benzoic acid (377 mg, 1.5 mmol) and HATU (684.4 mg, 1.8 mmol) in anhydrous DMF (2 m) was added propargyl-PEG4-amine (381.3 mg, 1.65 mmol) and DIEA (258.5 mg, 2 mmol). The reaction mixture was stirred at room temperature for 1 hour, then directly purified by RPLC (100 g, 5 to 100% acetonitrile and water, using 0.1% TFA as modifier). Yield 597.4 mg, 85.7%. Ion(s) found by LCMS: [M+H]⁺=465.0, [M−Boc+H]⁺=365.0.

### Step b

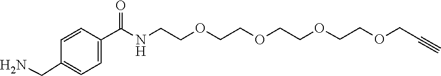 [SMILES: C#CCOCCOCCOCCOCCNC(=O)c1ccc(CN)cc1]

Product from step a (597.4 mg, 1.29 mmol) in acetonitrile (2 ml) was treated with 4N HCl solution in dioxane (1.5 ml). The reaction mixture was stirred at room temperature for 3 hours. It was then concentrated and dried under high vacuum. The crude product was carried to the subsequent step without purification. Ion(s) found by LCMS: [M+H]⁺=365.0.

### Step c

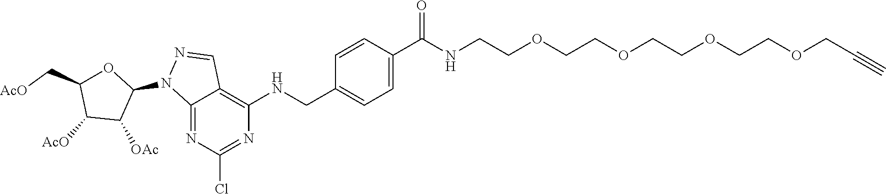 [SMILES: C#CCOCCOCCOCCOCCNC(=O)c1ccc(CNc2nc(Cl)nc3c2cnn3[C@@H]2O[C@H](COC(C)=O)[C@@H](OC(C)=O)[C@H]2OC(C)=O)cc1]

To a mixture of Intermediate B (305.8 mg, 0.684 mmol) and product from step b (302.5 mg, 0.752 mmol) in anhydrous ethanol (2 ml) was added triethylamine (276.3 mg, 2.74 mmol). The reaction mixture was heated at 50° C. for 1 hour, then cooled to room temperature and carried to the subsequent step without purification. Ion(s) found by LCMS: [M+H]⁺=774.6.

### Step d

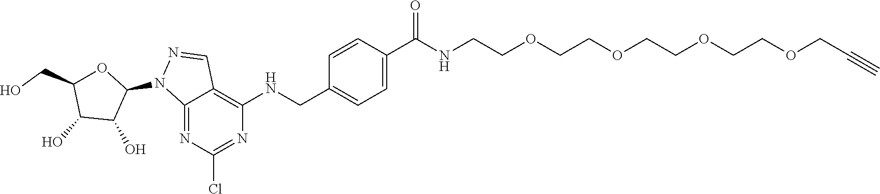 [SMILES: C#CCOCCOCCOCCOCCNC(=O)c1ccc(CNc2nc(Cl)nc3c2cnn3[C@@H]2O[C@H](CO)[C@@H](O)[C@H]2O)cc1]

To the crude solution from step c was added potassium carbonate (945.2 mg, 6.84 mmol) and methanol (2 ml). The resulting mixture was stirred at room temperature overnight. The salt was filtered off and washed with acetone. DOWEX 50W×8 hydrogen form (2 g) was added and stirred for 5 minutes. It was then filtered, and the filtrate was concentrated by rotary evaporation. The crude product was carried to the subsequent step without further purification. Ion(s) found by LCMS: [M+H]⁺=648.8.

### Step e

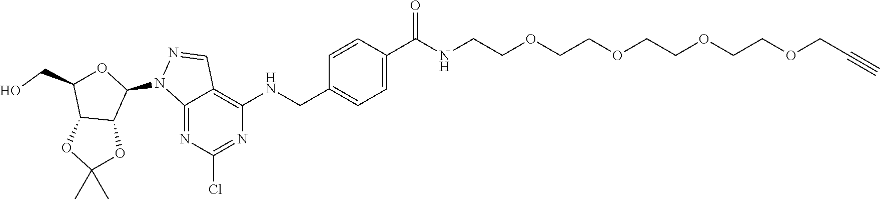 [SMILES: C#CCOCCOCCOCCOCCNC(=O)c1ccc(CNc2nc(Cl)nc3c2cnn3[C@@H]2O[C@H](CO)[C@H]3OC(C)(C)O[C@H]32)cc1]

To the crude product from step d was added acetone (5 ml), 2,2-dimethoxypropane (213.7 mg, 2.05 mmol) and p-TsOH (65 mg, 0.34 mmol). The resulting mixture was stirred at room temperature overnight. DIEA (260 mg, 2 mmol) was added, and the mixture was concentrated by rotary evaporation. The residue was purified by RPLC (100 g, 5 to 80% acetonitrile and water). Yield 335 mg, 71.1% over 3 steps. Ion(s) found by LCMS: [M+H]⁺=688.8.

### Step f

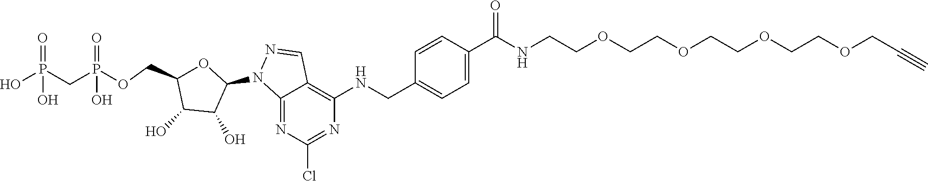 [SMILES: C#CCOCCOCCOCCOCCNC(=O)c1ccc(CNc2nc(Cl)nc3c2cnn3[C@@H]2O[C@H](COP(=O)(O)CP(=O)(O)O)[C@@H](O)[C@H]2O)cc1]

A flame-dried reaction flask was filled with nitrogen and charged with methylene(bis phosphonic dichloride) (273.1 mg, 1.09 mmol). The flask was cooled in an ice-water bath and added dropwise with a mixture of DIPEA (386.6 mg, 3 mmol) and product from step e (257.6 mg, 0.374 mmol) in anhydrous THE (2 ml). The reaction mixture was stirred at room temperature for 1 hour, then it was then cooled in an ice water bath. HCl aqueous solution (2 M, 1.5 ml) was added, and the reaction was stirred at room temperature overnight. THE was removed by rotary evaporation, and the remaining was purified by HPLC (5 to 50% acetonitrile and water, using 0.1% TFA as modifier). Yield 128.2 mg, 42.5%. Ion(s) found by LCMS: [M+H]⁺=806.6, [(M+2H)/2]⁺=403.8.

### Step g

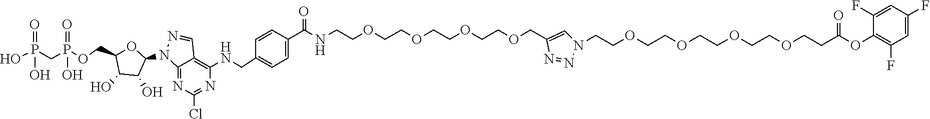 [SMILES: O=C(CCOCCOCCOCCOCCn1cc(COCCOCCOCCOCCNC(=O)c2ccc(CNc3nc(Cl)nc4c3cnn4[C@@H]3O[C@H](COP(=O)(O)CP(=O)(O)O)[C@@H](O)[C@H]3O)cc2)nn1)Oc1c(F)cc(F)cc1F]

Product from step f (20.3 mg, 0.022 mmol) was dissolved in DMF (0.4 ml) and methanol (0.6 ml), and the solution was cooled in an ice water bath. TFA (20 μl) was added, followed by Intermediate A (11.2 mg, 0.0265 mmol). A premixed THPTA (3.5 mg) and sodium ascorbate (20 mg) in water (0.3 ml) was added, then copper (II) sulfate (2 mg). The ice water bath was removed, and the reaction mixture was stirred for 1 hour. It was then directly purified by HPLC (5 to 70% acetonitrile and water, using 0.1% TFA as modifier). Yield 21.7 mg, 80.3%. Ion(s) found by LCMS: [M+H]⁺=1227.4, [M+2H)/2]⁺=614.2.

### Synthesis of Conjugate 23

To a solution of Fc SEQ ID NO: 13 (3.91 ml, 75.8 mg, 0.001304 mmol) in PBS 7.4 was added the product from Int-39 (14 mg, 0.01043 mmol) in DMF (0.255 ml). Additional DMF (0.25 ml×3) was used to wash the glassware and combined to the reaction mixture. The pH of the reaction mixture was slowly adjusted to ˜8.5 by 1M borate buffer solution (0.2 ml). The reaction was then gently rotated overnight and then purified according to the general procedure. Maldi TOF analysis of the purified final product gave an average mass of 64521 Da (DAR=5.8). Yield: 58.6 mg, 78.4%.

### Synthesis of Int-41

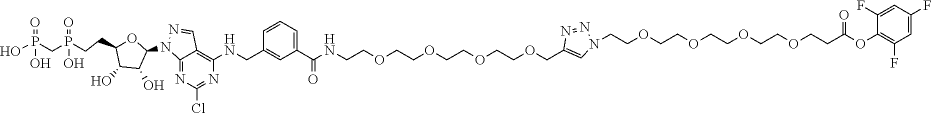 [SMILES: O=C(CCOCCOCCOCCOCCn1cc(COCCOCCOCCOCCNC(=O)c2cccc(CNc3nc(Cl)nc4c3cnn4[C@@H]3O[C@H](CCP(=O)(O)CP(=O)(O)O)[C@@H](O)[C@H]3O)c2)nn1)Oc1c(F)cc(F)cc1F]

The title compound was prepared analogously to Int-39, where 4-(Boc-aminomethyl)benzoic acid was replaced with 3-(N-Boc-aminomethyl)benzoic acid. Ion(s) found by LCMS: [M+H]⁺=1227.4, [(M+2H)/2]⁺=614.2.

### Synthesis of Conjugate 24

The title compound was prepared analogously to Conjugate 23, where the product described in Int-39 was replaced with Int-41 Maldi TOF analysis of the purified final product gave an average mass of 64,484 Da (DAR=5.8). Yield: 48.2 mg, 78.9%.

### Synthesis of Int-43

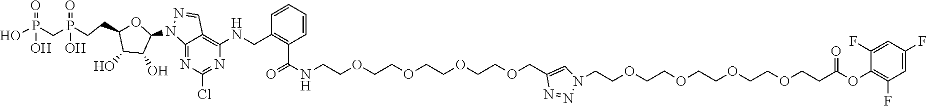 [SMILES: O=C(CCOCCOCCOCCOCCn1cc(COCCOCCOCCOCCNC(=O)c2ccccc2CNc2nc(Cl)nc3c2cnn3[C@@H]2O[C@H](CCP(=O)(O)CP(=O)(O)O)[C@@H](O)[C@H]2O)nn1)Oc1c(F)cc(F)cc1F]

The title compound was prepared analogously to Int-39, where 4-(Boc-aminomethyl)benzoic acid was replaced with 2-(Boc-aminomethyl)benzoic acid. Ion(s) found by LCMS: [M+H]⁺=1227.4, [(M+2H)/2]⁺=614.2.

### Synthesis of Conjugate 25

The title compound was prepared analogously to Conjugate 23, where the product described in Int-39 was replaced with Int-43. Maldi TOF analysis of the purified final product gave an average mass of 64523 Da (DAR=5.8). Yield: 39.6 mg, 76.2%.

### Synthesis of Int-63

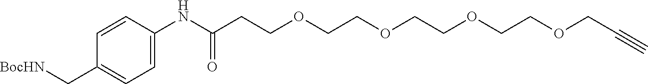 [SMILES: C#CCOCCOCCOCCOCCC(=O)Nc1ccc(CNC(=O)OC(C)(C)C)cc1]

### Step a

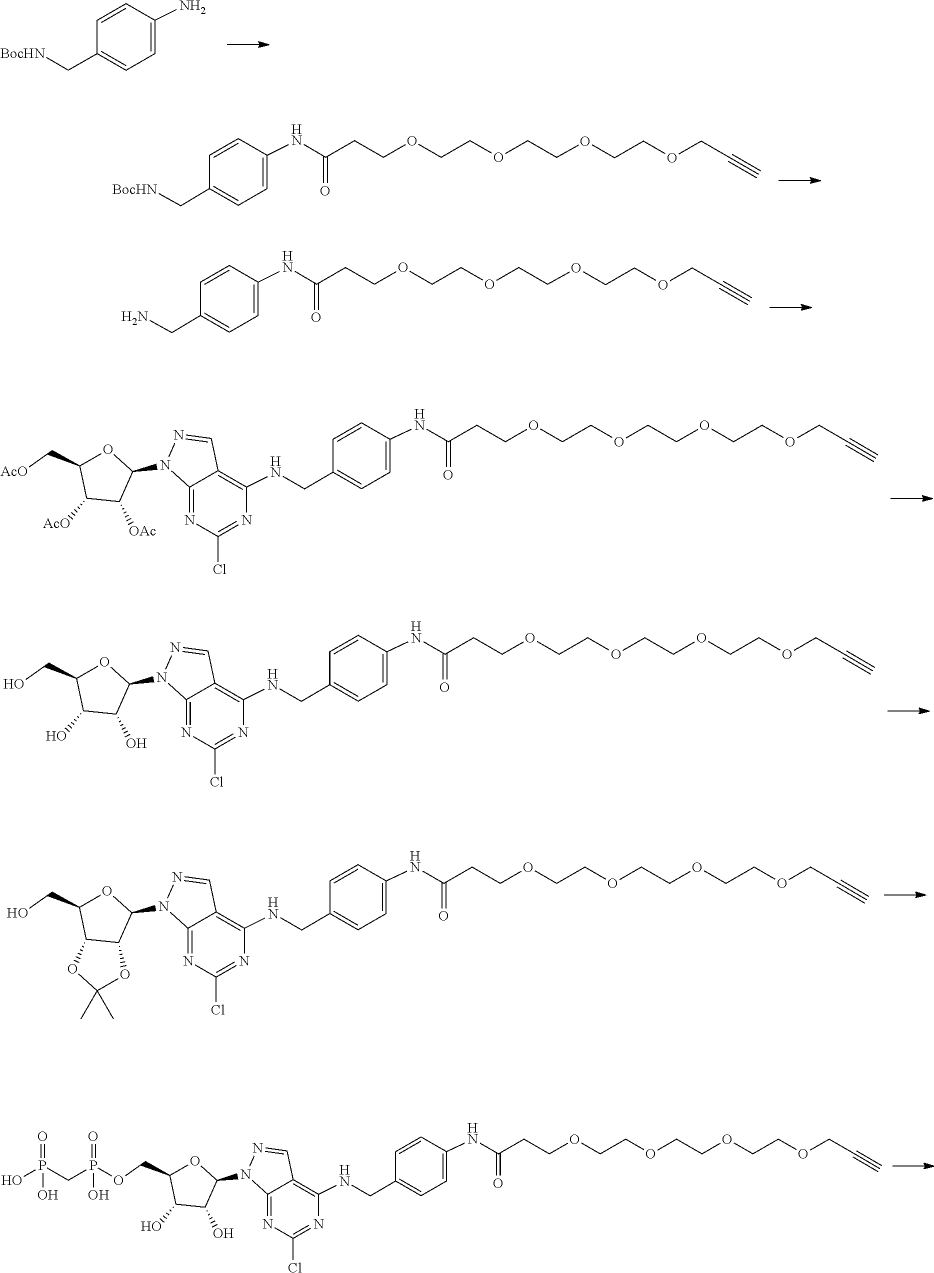

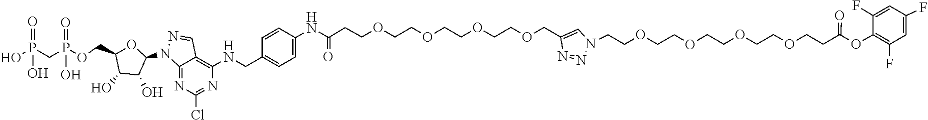 [SMILES: O=C(CCOCCOCCOCCOCc1cn(CCOCCOCCOCCOCCC(=O)Oc2c(F)cc(F)cc2F)nn1)Nc1ccc(CNc2nc(Cl)nc3c2cnn3[C@@H]2O[C@H](COP(=O)(O)CP(=O)(O)O)[C@@H](O)[C@H]2O)cc1]

To a solution of propargyl-PEG4-acid (390.5 mg, 1.5 mmol) in anhydrous DMF (2.0 ml) was added HOBT hydrate (275.6 mg, 1.8 mmol), EDC HCl (343.8 mg, 1.8 mmol) and 4-(N-Boc-aminomethyl)aniline (400 mg, 1.8 mmol). The resulting mixture was stirred at room temperature overnight. It was then directly purified by RPLC (100 g, 5 to 100% acetonitrile and water, using 0.1% TFA as modifier). Yield 637.9 mg, 91.5%. Ion(s) found by LCMS: [M+H]⁺=465.0.

### Step b

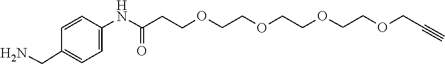 [SMILES: C#CCOCCOCCOCCOCCC(=O)Nc1ccc(CN)cc1]

Product from step a (638 mg, 1.37 mmol) was dissolved in acetonitrile (3 ml) and treated with 6N HCl aqueous solution (1 ml). The reaction mixture was heated at 50° C. for 1 hour, then concentrated by rotary evaporation and dried under high vacuum. The crude product was carried to the subsequent step without further purification. Ion(s) found by LCMS: [M+H]⁺=365.0.

### Step c

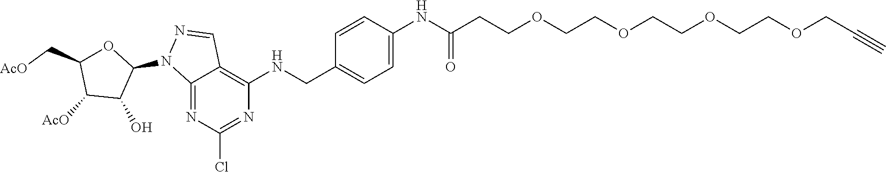 [SMILES: C#CCOCCOCCOCCOCCC(=O)Nc1ccc(CNc2nc(Cl)nc3c2cnn3[C@@H]2O[C@H](COC(C)=O)[C@@H](OC(C)=O)[C@H]2O)cc1]

To a mixture of Intermediate B (305.8 mg, 0.684 mmol) and product from the previous step (302.5 mg, 0.752 mmol) in anhydrous ethanol (2 ml) was added triethylamine (276.3 mg, 2.74 mmol). The reaction mixture was heated at 50° C. for 1 hour, then cooled to room temperature and carried to the next step without purification. Ion(s) found by LCMS: [M+H]⁺=774.6.

### Step d

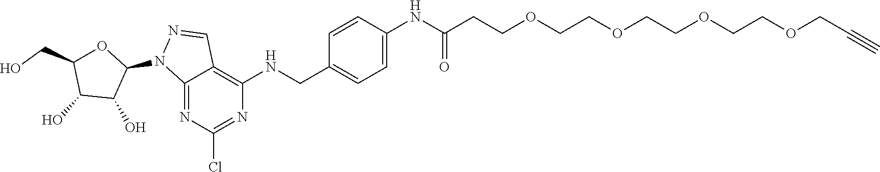 [SMILES: C#CCOCCOCCOCCOCCC(=O)Nc1ccc(CNc2nc(Cl)nc3c2cnn3[C@@H]2O[C@H](CO)[C@@H](O)[C@H]2O)cc1]

To the crude solution from step c was added potassium carbonate (945.2 mg, 6.84 mmol) and methanol (2 ml). The resulting mixture was stirred at room temperature overnight. The salt was filtered off and washed with acetone. DOWEX 50W×8 hydrogen form (2 g) was added and stirred for 5 minutes. It was then filtered, and the filtrate was concentrated by rotary evaporation. The crude product was carried to the subsequent step without further purification. Ion(s) found by LCMS: [M+H]⁺=648.8.

### Step e

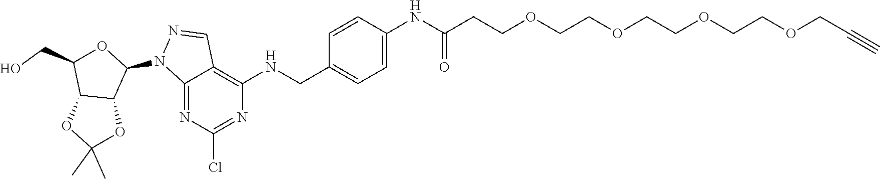 [SMILES: C#CCOCCOCCOCCOCCC(=O)Nc1ccc(CNc2nc(Cl)nc3c2cnn3[C@@H]2O[C@H](CO)[C@H]3OC(C)(C)O[C@H]32)cc1]

To the crude product from step d was added acetone (5 ml), 2,2-dimethoxypropane (213.7 mg, 2.05 mmol) and p-TsOH (65 mg, 0.34 mmol). The resulting mixture was stirred at room temperature overnight. DIEA (260 mg, 2 mmol) was added, and the mixture was concentrated by rotary evaporation. The residue was purified by RPLC (100 g, 5 to 80% acetonitrile and water). Yield 350.7 mg, 74.4% over 3 steps. Ion(s) found by LCMS: [M+H]⁺=688.8.

### Step f

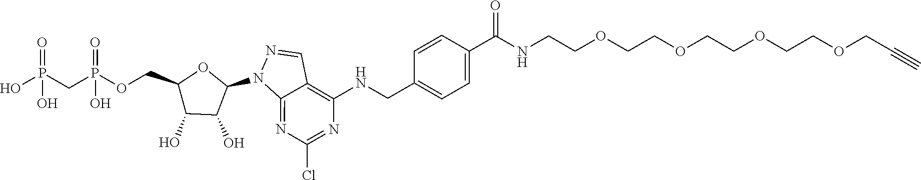 [SMILES: C#CCOCCOCCOCCOCCNC(=O)c1ccc(CNc2nc(Cl)nc3c2cnn3[C@@H]2O[C@H](COP(=O)(O)CP(=O)(O)O)[C@@H](O)[C@H]2O)cc1]

A flame-dried reaction flask was filled with nitrogen and charged with methylene(bis phosphonic dichloride) (273.1 mg, 1.09 mmol). The flask was cooled in an ice-water bath and added dropwise with a mixture of DIEA (386.6 mg, 3 mmol) and product from step e (257.6 mg, 0.374 mmol) in anhydrous THE (2 ml). After the reaction mixture was stirred at room temperature for 1 hour, it was then cooled in an ice water bath. HCl aqueous solution (2 M, 1.5 ml) was added, and the reaction was stirred at room temperature overnight. THE was removed by rotary evaporation, and the remaining was purified by reversed phase HPLC (5 to 50% acetonitrile and water, using 0.1% TFA as modifier). Yield 54 mg, 17.9%. Ion(s) found by LCMS: [M+H]⁺=806.6, [M+2H)/2]⁺=403.8.

### Step g

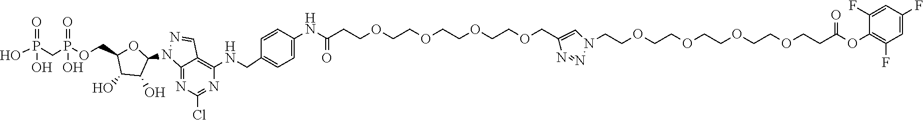 [SMILES: O=C(CCOCCOCCOCCOCc1cn(CCOCCOCCOCCOCCC(=O)Oc2c(F)cc(F)cc2F)nn1)Nc1ccc(CNc2nc(Cl)nc3c2cnn3[C@@H]2O[C@H](COP(=O)(O)CP(=O)(O)O)[C@@H](O)[C@H]2O)cc1]

Product from step f (20.4 mg, 0.0252 mmol) was dissolved in DMF (0.3 ml) and methanol (0.5 ml), and the solution was cooled in an ice water bath. TFA (20 μl) was added, followed by Intermediate A (11.2 mg, 0.0265 mmol). A premixed THPTA (3.5 mg) and sodium ascorbate (20 mg) in water (0.3 ml) was added, followed by copper (II) sulfate (2 mg). The ice water bath was removed, and the reaction mixture was stirred for 1 hour. It was then directly purified by HPLC: 5 to 70% acetonitrile and water, using 0.1% TFA as modifier. Yield 23.2 mg, 75.0%. Ion(s) found by LCMS: [M+H]⁺=1227.4, [M+2H)/2]⁺=614.2.

### Synthesis of Conjugate 35

To a solution of Fc SEQ ID NO: 13 (3.723 ml, 72.2 mg, 0.001241 mmol) in PBS 7.4 was added DMF (0.6 ml) and the product described in Int-63 (12.2 mg, 0.00993 mmol) in DMF (0.2 ml). Additional DMF (0.2 ml×3) was used to wash the glassware and combined to the reaction mixture. The pH of the reaction mixture was slowly adjusted to ˜8.5 by 1M borate buffer solution (0.1 ml×2). The reaction was then gently rotate for 4 hours and purified according to the general procedure. Maldi TOF analysis of the purified final product gave an average mass of 63,152 Da (DAR=4.6). Yield: 52.3 mg, 72.5%.

### Synthesis of Int-65

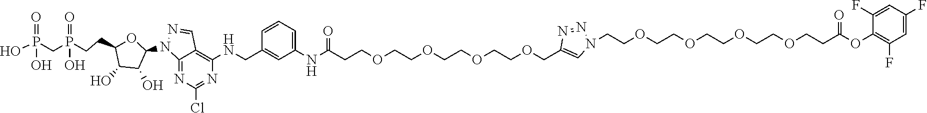 [SMILES: O=C(CCOCCOCCOCCOCc1cn(CCOCCOCCOCCOCCC(=O)Oc2c(F)cc(F)cc2F)nn1)Nc1cccc(CNc2nc(Cl)nc3c2cnn3[C@@H]2O[C@H](CCP(=O)(O)CP(=O)(O)O)[C@@H](O)[C@H]2O)c1]

The title compound was prepared analogously to Int-63, where 4-(N-Boc-aminomethyl)aniline was replaced with tert-butyl 3-aminobenzylcarbamate. Ion(s) found by LCMS: [M+H]⁺=1227.4, [(M+2H)/2]⁺=614.2.

### Synthesis of Conjugate 36

The title compound was prepared analogously to Conjugate 35, where the product described in Int-63 was replaced with Int-65. Maldi TOF analysis of the purified final product gave an average mass of 63,152 Da (DAR=4.6). Yield: 52.1 mg, 77.2%.

### Synthesis of Int-67

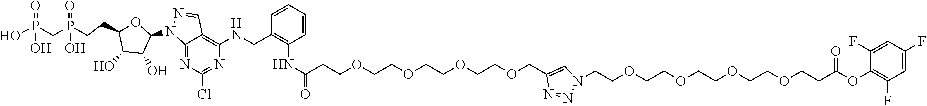 [SMILES: O=C(CCOCCOCCOCCOCc1cn(CCOCCOCCOCCOCCC(=O)Oc2c(F)cc(F)cc2F)nn1)Nc1ccccc1CNc1nc(Cl)nc2c1cnn2[C@@H]1O[C@H](CCP(=O)(O)CP(=O)(O)O)[C@@H](O)[C@H]1O]

The title compound was prepared analogously to Int-63, where 4-(N-Boc-aminomethyl)aniline was replaced with (2-amino-benzyl)-carbamic acid tert-butyl ester. Ion(s) found by LCMS: [M+H]⁺=1227.4, [(M+2H)/2]⁺=614.2.

### Synthesis of Conjugate 37

The title compound was prepared analogously to Conjugate 35, where the product described in Int-63 was replaced with Int-67. Maldi TOF analysis of the purified final product gave an average mass of 63312 Da (DAR=4.7). Yield: 51.4 mg, 78.9%.

### Synthesis of Int-83

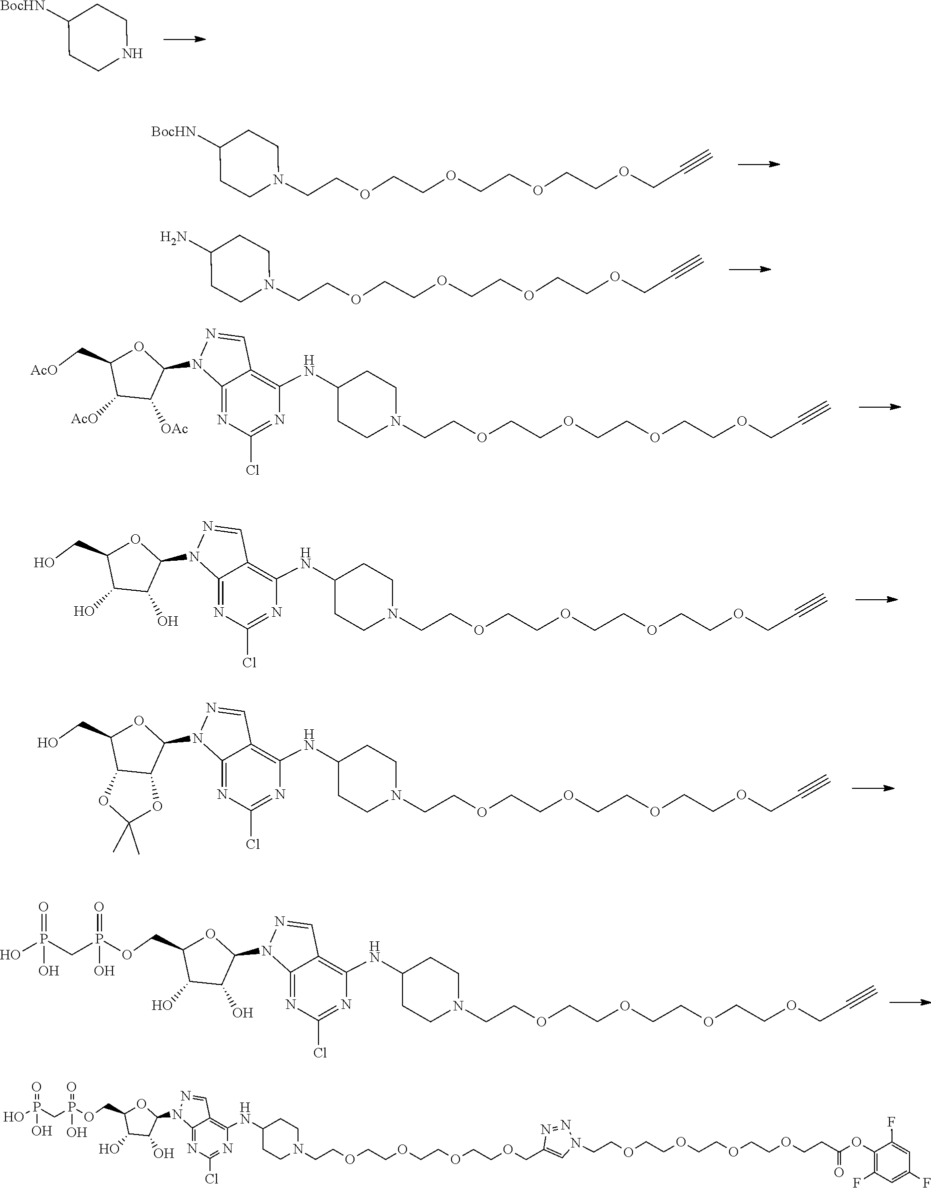

### Step a

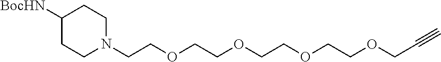 [SMILES: C#CCOCCOCCOCCOCCN1CCC(NC(=O)OC(C)(C)C)CC1]

To a solution of 4-Boc-aminopiperidine (300.5 mg, 1.5 mmol) in anhydrous DMF (1 ml) was added potassium carbonate (414.6 mg, 3 mmol), propargyl-PEG4-mesyl ester (698.4 mg, 2.25 mmol) and anhydrous dioxane (2 ml). The resulting mixture was heated at 60° C. for 2 days. The salt was filtered off and washed with acetonitrile. The filtrate was concentrated by rotary evaporation and purified by RPLC (100 g, 0 to 60% acetonitrile and water, using 0.1% TFA as modifier). Yield 720.8 mg, 90.9%. Ion(s) found by LCMS: [M+H]⁺=415.0.

### Step b

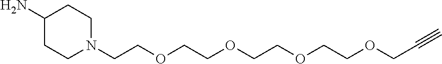 [SMILES: C#CCOCCOCCOCCOCCN1CCC(N)CC1]

Product from step a (720.9 mg, 1.36 mmol) was dissolved in acetonitrile (5 ml) and treated with 6N HCl aqueous solution (1 ml). The reaction mixture was heated at 50° C. for 1 hour, then concentrated by rotary evaporation and dried under high vacuum. The crude product was carried to the next step without further purification. Ion(s) found by LCMS: [M+H]⁺=315.0.

### Step c

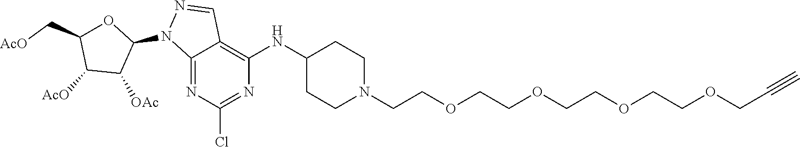 [SMILES: C#CCOCCOCCOCCOCCN1CCC(Nc2nc(Cl)nc3c2cnn3[C@@H]2O[C@H](COC(C)=O)[C@@H](OC(C)=O)[C@H]2OC(C)=O)CC1]

To a mixture of Intermediate B (305.8 mg, 0.684 mmol) and product from step b (293.3 mg, 0.752 mmol) in anhydrous ethanol (2 ml) was added triethylamine (276.3 mg, 2.74 mmol). The reaction mixture was heated at 50° C. for 1 hour, then cooled to room temperature and carried to the subsequent step without purification. Ion(s) found by LCMS: [M+H]⁺=724.6.

### Step d

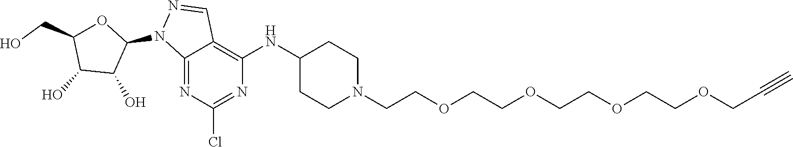 [SMILES: C#CCOCCOCCOCCOCCN1CCC(Nc2nc(Cl)nc3c2cnn3[C@@H]2O[C@H](CO)[C@@H](O)[C@H]2O)CC1]

To the crude solution from step c was added potassium carbonate (472.6 mg, 3.42 mmol) and methanol (2 ml). The resulting mixture was stirred at room temperature overnight. The salt was filtered off and washed with acetone. DOWEX 50W×8 hydrogen form (2 g) was added and stirred for 5 minutes. It was then filtered, and the filtrate was concentrated by rotary evaporation. The crude product was carried to the subsequent step without further purification. Ion(s) found by LCMS: [M+H]⁺=598.8, [(M+2H)/2]⁺=300.0.

### Step e

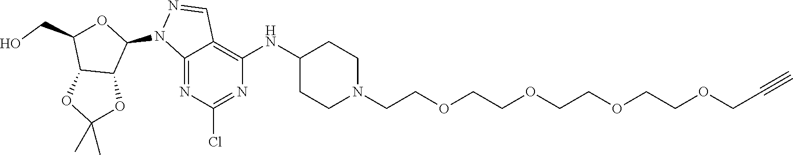 [SMILES: C#CCOCCOCCOCCOCCN1CCC(Nc2nc(Cl)nc3c2cnn3[C@@H]2O[C@H](CO)[C@H]3OC(C)(C)O[C@H]32)CC1]

To the crude product from step d was added NMP (1 ml), acetone (5 ml), 2,2-dimethoxypropane (213.7 mg, 2.05 mmol) and p-TsOH (65 mg, 0.34 mmol). The resulting mixture was stirred at room temperature overnight. DIPEA (260 mg, 2 mmol) was added, and the mixture was concentrated by rotary evaporation. The residue was purified by RPLC (100 g, 5 to 60% acetonitrile and water). Yield 140 mg, 32% over 3 steps. Ion(s) found by LCMS: [M+H]⁺=638.8.

### Step f

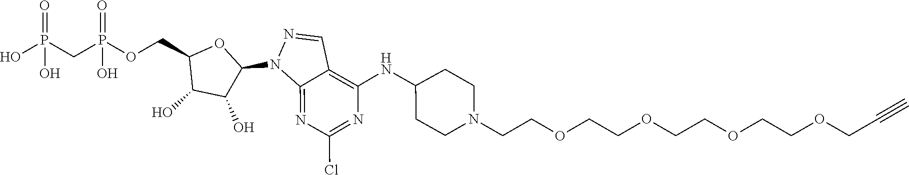 [SMILES: C#CCOCCOCCOCCOCCN1CCC(Nc2nc(Cl)nc3c2cnn3[C@@H]2O[C@H](COP(=O)(O)CP(=O)(O)O)[C@@H](O)[C@H]2O)CC1]

A flame-dried reaction flask was filled with nitrogen and charged with methylene (bis phosphonic dichloride) (109.4 mg, 0.439 mmol). The flask was cooled in an ice-water bath and added dropwise with a mixture of DIEA (34.2 mg, 0.263 mmol) and product from step e (140 mg, 0.219 mmol) in anhydrous THE (1 ml). Addition THE (1 ml) was used to rinse the flask and combined to the reaction mixture. After the reaction mixture was stirred at room temperature for 1 hour, it was then cooled in an ice water bath. A solution of pre-mixed HCl aqueous solution (6 N, 0.5 ml) and water (1 ml) was added, and the reaction was stirred at room temperature overnight. THE was removed by rotary evaporation, and the remaining crude product was purified by reversed phase HPLC (5 to 50% acetonitrile and water, using 0.1% TFA as modifier). Yield 64.6 mg, 33.9%. Ion(s) found by LCMS: [M+H]⁺=756.6, [(M+2H)/2]⁺=378.9.

### Step g

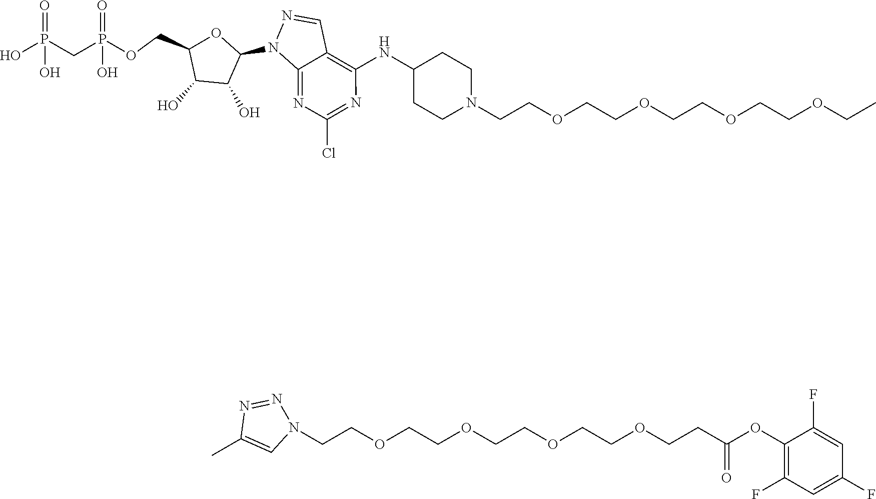

Product from step f (21.4 mg, 0.0246 mmol) was dissolved in DMF (0.4 ml) and methanol (0.6 ml), and the solution was cooled in an ice water bath. TFA (20 μl) was added, followed by Intermediate A (12.5 mg, 0.0295 mmol). A premixed THPTA (3.5 mg) and sodium ascorbate (20 mg) in water (0.3 ml) was added, followed by copper(II)sulfate (2 mg). The ice water bath was removed, and the reaction mixture was stirred for 1 hour. It was then directly purified by reversed phase HPLC: 5 to 70% acetonitrile and water, using 0.1% TFA as modifier. Yield 25 mg, 78.6%. Ion(s) found by LCMS: [M+H]⁺=1177.4, [(M+2H)/2]⁺=589.5, [(M+3H)/3]⁺=393.8.

### Synthesis of Conjugate 45

To a solution of Fc SEQ ID NO: 13 (4.35 ml, 84.4 mg, 0.001451 mmol) in PBS 7.4 was added Int-83 (15 mg, 0.01161 mmol) in DMF (0.2 ml). Additional DMF (0.2 ml×3) was used to wash the glassware and combined to the reaction mixture. The pH of the reaction mixture was slowly adjusted to ˜8.5 by 1M borate buffer solution (0.1 ml×2). The reaction was then gently rotated for 4 hours and then purified according to the general procedure. Maldi TOF analysis of the purified final product gave an average mass of 63226 Da (DAR=4.9). Yield: 61.2 mg, 76.5%.

### Synthesis of Int-85

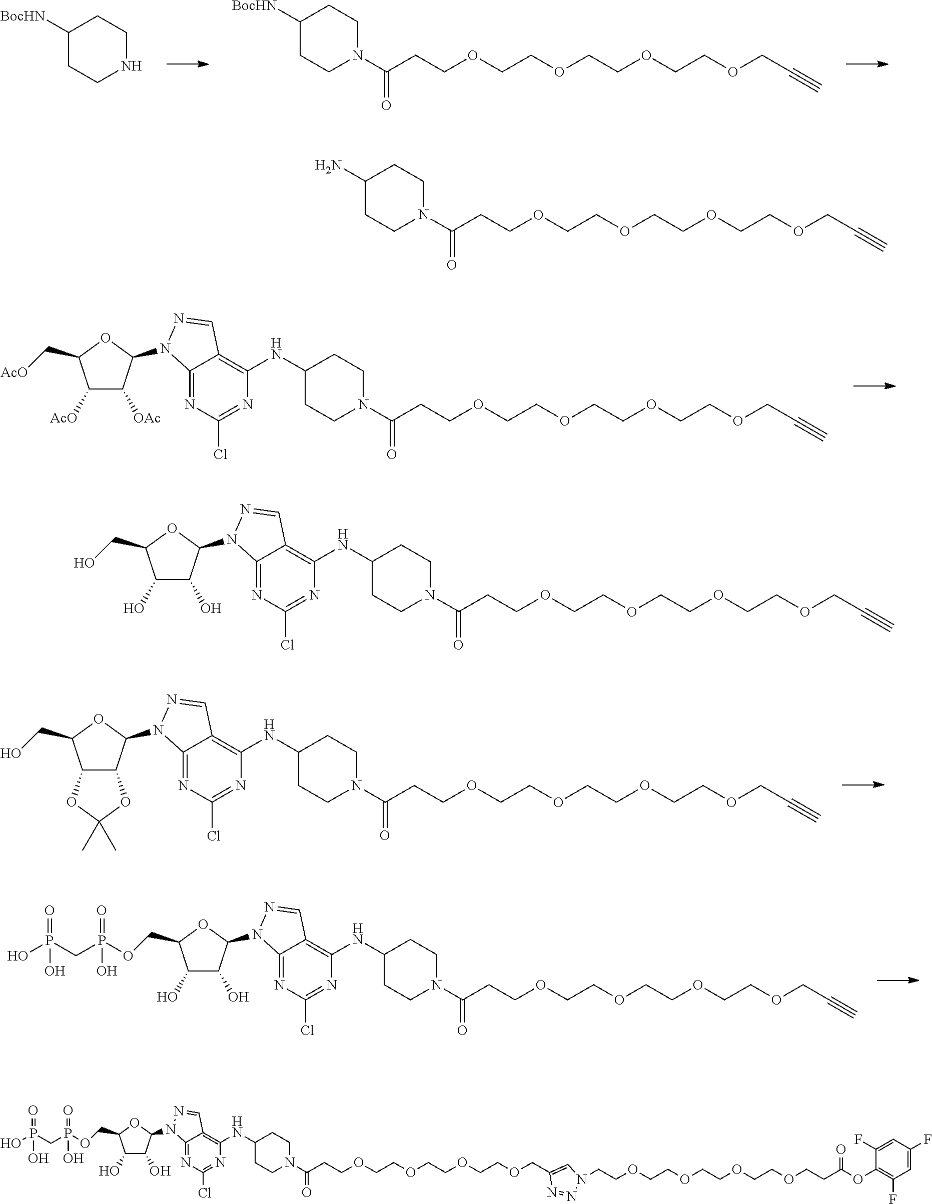

### Step a

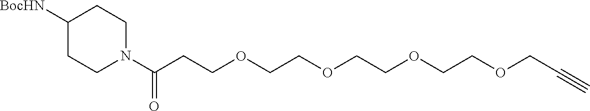 [SMILES: C#CCOCCOCCOCCOCCC(=O)N1CCC(NC(=O)OC(C)(C)C)CC1]

To a solution of propargyl-PEG4-acid acid (390.5 mg, 1.5 mmol) and HATU (684.4 mg, 1.8 mmol) in anhydrous DMF (2 m) was added 4-Boc-aminopiperidine (330.5 mg, 1.65 mmol) and DIEA (258.5 mg, 2 mmol). The reaction mixture was stirred at room temperature for 1 hour, then directly purified by RPLC (100 g, 5 to 80% acetonitrile and water, using 0.1% TFA as modifier). Yield 586.4 mg, 88%. Ion(s) found by LCMS: [M+H]⁺=443.0.

### Step b

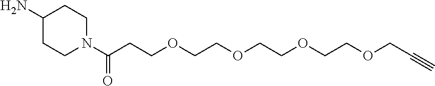 [SMILES: C#CCOCCOCCOCCOCCC(=O)N1CCC(N)CC1]

Product from step a (586.4 mg, 1.32 mmol) was dissolved in acetonitrile (2 ml) and treated with 6N HCl aqueous solution (1 ml). The reaction mixture was heated at 50° C. for 1 hour, then concentrated by rotary evaporation and dried under high vacuum. The crude product was carried to the next step without further purification. Ion(s) found by LCMS: [M+H]⁺=343.0.

### Step c

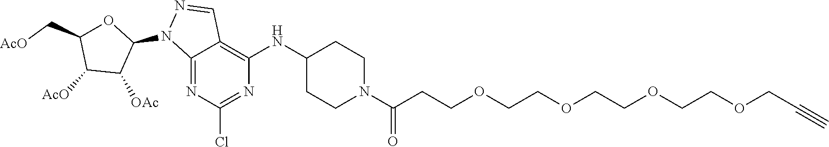 [SMILES: C#CCOCCOCCOCCOCCC(=O)N1CCC(Nc2nc(Cl)nc3c2cnn3[C@@H]2O[C@H](COC(C)=O)[C@@H](OC(C)=O)[C@H]2OC(C)=O)CC1]

To a mixture of Intermediate B (305.8 mg, 0.684 mmol) and product from step b (314 mg, 0.752 mmol) in anhydrous ethanol (2 ml) was added triethylamine (276.3 mg, 2.74 mmol). The reaction mixture was heated at 50° C. for 1 hour, then cooled to room temperature and carried to the next step without purification. Ion(s) found by LCMS: [M+H]⁺=752.6.

### Step d

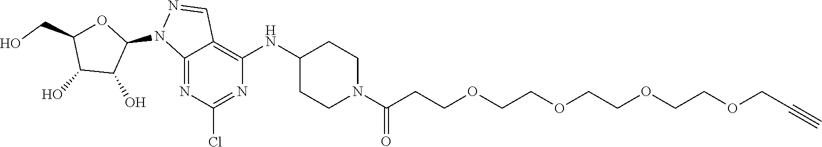 [SMILES: C#CCOCCOCCOCCOCCC(=O)N1CCC(Nc2nc(Cl)nc3c2cnn3[C@@H]2O[C@H](CO)[C@@H](O)[C@H]2O)CC1]

To the crude solution from step c was added potassium carbonate (472.6 mg, 3.42 mmol) and methanol (2 ml). The resulting mixture was stirred at room temperature overnight. The salt was filtered off and washed with acetone. DOWEX 50W×8 hydrogen form (2 g) was added and stirred for 5 minutes. It was then filtered, and the filtrate was concentrated by rotary evaporation. The crude product was carried to the subsequent step without further purification. Ion(s) found by LCMS: [M+H]⁺=626.8.

### Step e

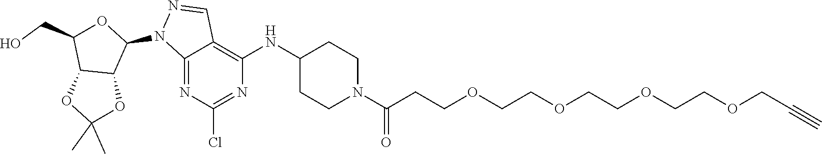 [SMILES: C#CCOCCOCCOCCOCCC(=O)N1CCC(Nc2nc(Cl)nc3c2cnn3[C@@H]2O[C@H](CO)[C@H]3OC(C)(C)O[C@H]32)CC1]

To the crude product from step d was added acetone (6 ml), 2,2-dimethoxypropane (213.7 mg, 2.05 mmol) and p-TsOH (65 mg, 0.34 mmol). The resulting mixture was stirred at room temperature overnight. DIEA (260 mg, 2 mmol) was added, and the mixture was concentrated by rotary evaporation. The residue was purified by RPLC (100 g, 5 to 60% acetonitrile and water). Yield 267.7 mg, 59% over 3 steps. Ion(s) found by LCMS: [M+H]⁺=666.8.

### Step f

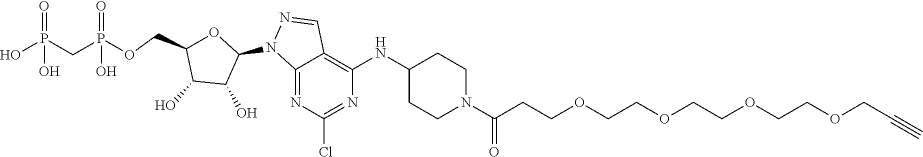 [SMILES: C#CCOCCOCCOCCOCCC(=O)N1CCC(Nc2nc(Cl)nc3c2cnn3[C@@H]2O[C@H](COP(=O)(O)CP(=O)(O)O)[C@@H](O)[C@H]2O)CC1]

A flame-dried reaction flask was filled with nitrogen and charged with methylene(bis phosphonic dichloride) (120.3 mg, 0.481 mmol). The flask was cooled in an ice-water bath and added with a pre-cooled mixture of DIEA (65 mg, 0.5 mmol) and product from step e (267.7 mg, 0.401 mmol) in anhydrous THE (1 ml). Addition THE (1 ml) was used to rinse the flask and combined to the reaction mixture. After the reaction mixture was stirred at room temperature for 1 hour, it was then cooled to 0° C. by an ice water bath. A solution of pre-mixed HCl aqueous solution (6 N, 0.5 ml) and water (1 ml) was added, and the reaction was stirred at room temperature overnight. THE was removed by rotary evaporation, and the remaining was purified by reversed phase HPLC: 5 to 50% acetonitrile and water, using 0.1% TFA as modifier). Yield 150 mg, 47.6%. Ion(s) found by LCMS: [M+H]⁺=784.6, [(M+2H)/2]⁺=392.8.

### Step g

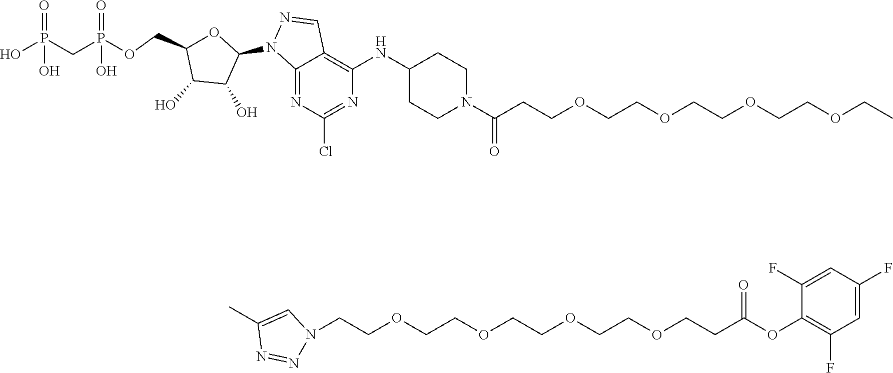

Product from step g (25.3 mg, 0.0322 mmol) was dissolved in DMF (0.4 ml) and methan or (0.6 ml), and the solution was cooled in an ice water bath. TFA (40 μl) was added, followed by Intermediate A (21.4 mg, 0.0483 mmol). A premixed THPTA (3.5 mg) and sodium ascorbate (20 mg) in water (0.3 ml) was added, then copper(II)sulfate (2 mg). The ice water bath was removed, and the reaction mixture was stirred for 1 hour. It was then directly purified by HPLC: 5 to 70% acetonitrile and water, using 0.1% TFA as modifier. Yield 25.5 mg, 65.6%. Ion(s) found by LCMS: [(M+2H)/2]⁺=603.1.

### Synthesis of Conjugate 46

The title compound was prepared analogously to Conjugate 34, where the product from Int-83 was replaced with Int-85. Maldi TOF analysis of the purified final product gave an average mass of 62369 Da (DAR=3.9). Yield: 74.0 mg, 74.8%.

### Synthesis of Int-102

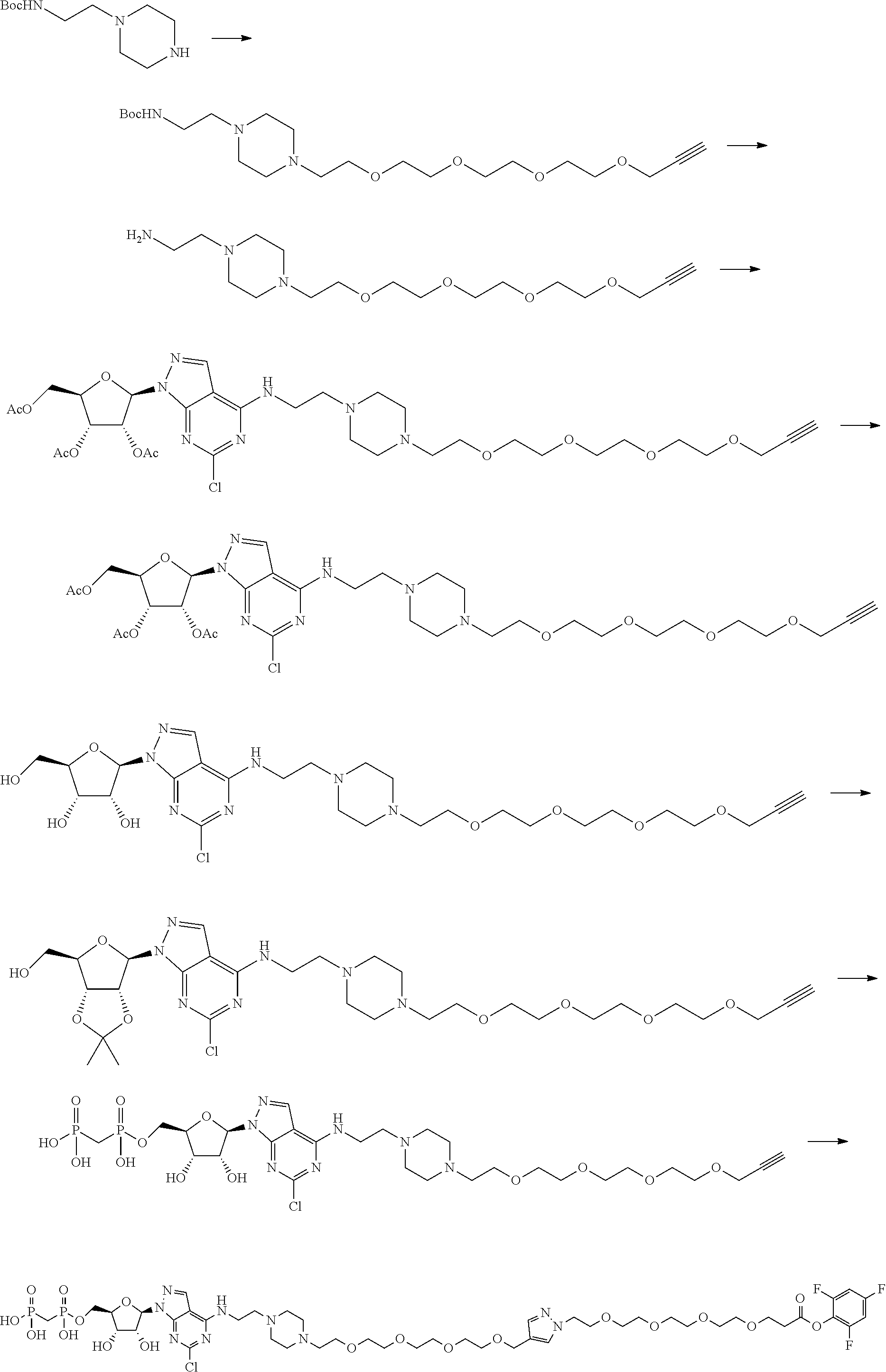

### Step a

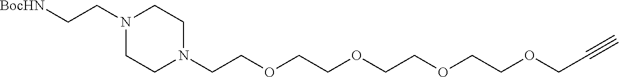 [SMILES: C#CCOCCOCCOCCOCCN1CCN(CCNC(=O)OC(C)(C)C)CC1]

To a solution of 1-((2-N-Boc-amino)ethyl)piperazine (1.147 g, 5 mmol) in anhydrous DMF (7.5 ml) was added potassium carbonate (1.04 g, 7.5 mmol), propargyl-PEG4-mesyl ester (2.33 g, 7.5 mmol) and anhydrous dioxane (2 ml). The resulting mixture was heated at 70° C. for 1 day. The salt was filtered off and washed with acetonitrile. The filtrate was concentrated by rotary evaporation and purified by RPLC (5 to 80% acetonitrile and water, using 0.1 % TFA as modifier). Yield 1.67 g, 49.7%. Ion(s) found by LCMS: [M+H]⁺=444.0.

### Step b

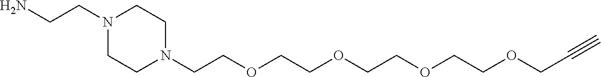 [SMILES: C#CCOCCOCCOCCOCCN1CCN(CCN)CC1]

Product from step a was dissolved in acetonitrile (3 ml) and treated with 6N HCl aqueous solution (3 ml). The mixture was heated at 50° C. for 1 hour. It was then lyophilized. The crude product was carried to the subsequent step without purification. Ion(s) found by LCMS: [M+H]⁺=343.0.

### Step c

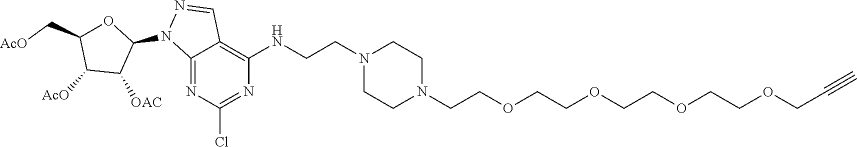

To a mixture of Intermediate B (305.8 mg, 0.684 mmol) and product from step b (314 mg, 0.752 mmol) in anhydrous ethanol (2 ml) was added triethylamine (276.3 mg, 2.74 mmol). The reaction mixture was heated at 50° C. for 1 hour, then cooled to room temperature and carried to the next step without purification. Ion(s) found by LCMS: [M+H]⁺=753.8.

### Step d

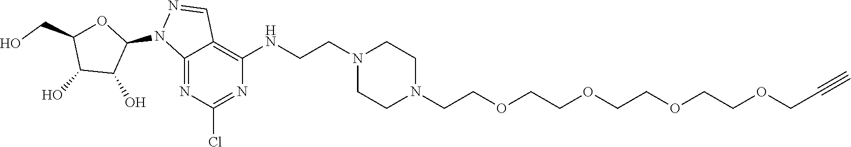 [SMILES: C#CCOCCOCCOCCOCCN1CCN(CCNc2nc(Cl)nc3c2cnn3[C@@H]2O[C@H](CO)[C@@H](O)[C@H]2O)CC1]

To the crude solution from step c was added potassium carbonate (472.6 mg, 3.42 mmol) and methanol (2 ml). The resulting mixture was stirred at room temperature overnight. The salt was filtered off and washed with acetone. DOWEX 50W×8 hydrogen form (2.5 g) was added and stirred for 5 minutes. It was then filtered, and the filtrate was concentrated by rotary evaporation. The crude product was carried to the subsequent step without further purification. Ion(s) found by LCMS: [M+H]⁺=627.4, [(M+2H)/2]⁺=314.2.

### Step e

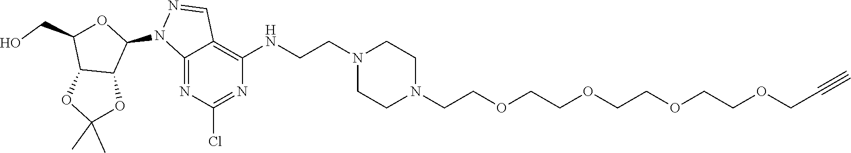 [SMILES: C#CCOCCOCCOCCOCCN1CCN(CCNc2nc(Cl)nc3c2cnn3[C@@H]2O[C@H](CO)[C@H]3OC(C)(C)O[C@H]32)CC1]

To the crude product from step d in anhydrous NMP (1 ml) was added acetone (6 ml), 2,2-dimethoxypropane (1.05 g, 9.3 mmol) and p-TsOH (236 mg, 1.24 mmol). The resulting mixture was stirred at room temperature overnight. DIEA (260 mg, 2 mmol) was added, and the mixture was concentrated by rotary evaporation. The residue was purified by RPLC (100 g, 0 to 70% acetonitrile and water). Yield 160.5 mg, 35.1% over 3 steps. Ion(s) found by LCMS: [M+H]⁺=668.2.

### Step f

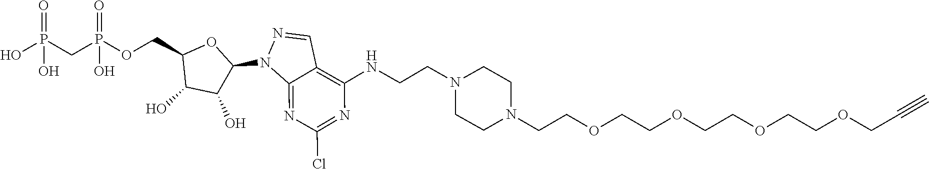 [SMILES: C#CCOCCOCCOCCOCCN1CCN(CCNc2nc(Cl)nc3c2cnn3[C@@H]2O[C@H](COP(=O)(O)CP(=O)(O)O)[C@@H](O)[C@H]2O)CC1]

A flame-dried reaction flask was filled with nitrogen and charged with methylene (bis phosphonic dichloride) (180 mg, 0.72 mmol). The flask was cooled in an ice-water bath and added to a pre-cooled solution of product from step e (160.5 mg, 0.24 mmol) in anhydrous THE (1 ml). Addition THE (1 ml) was used to rinse the flask and combined to the reaction mixture. After the reaction mixture was stirred at room temperature for 1 hour, it was then cooled to 0° C. by an ice water bath. A solution of pre-mixed HCl aqueous solution (6 N, 0.5 ml) and water (1 ml) was added, and the reaction was stirred at room temperature overnight. THE was removed by rotary evaporation, and the remaining was purified by reversed phase HPLC (5 to 50% acetonitrile and water, using 0.1% TFA as modifier). Yield 63.3 mg, 26%. Ion(s) found by LCMS: [M+H]⁺=786.2, [(M+2H)/2]⁺=393.2.

### Step g

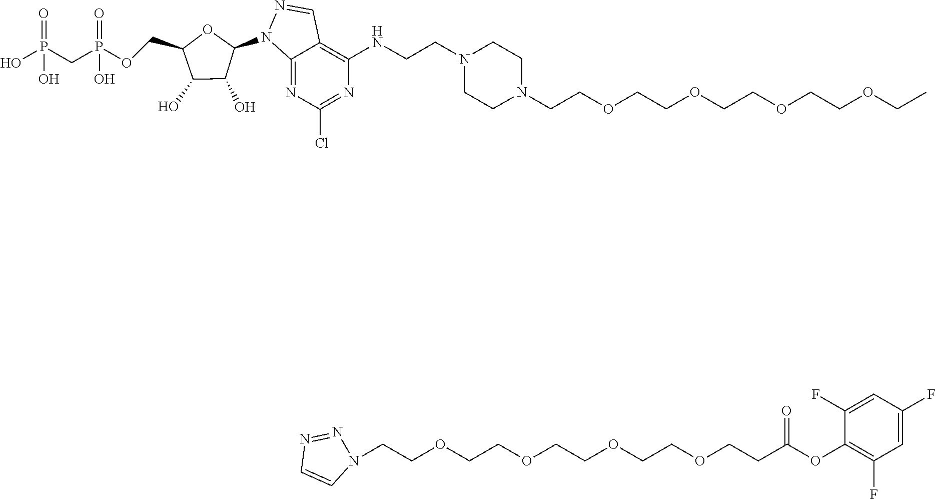 [SMILES: CCOCCOCCOCCOCCN1CCN(CCNc2nc(Cl)nc3c2cnn3[C@@H]2O[C@H](COP(=O)(O)CP(=O)(O)O)[C@@H](O)[C@H]2O)CC1 ; O=C(CCOCCOCCOCCOCCn1ccnn1)Oc1c(F)cc(F)cc1F]

Product from step f (22.4 mg, 0.0221 mmol) was dissolved in DMF (0.4 ml) and methanol (0.4 ml), and the solution was cooled in an ice water bath. TFA (40 μl) was added, followed by Intermediate A (14.6 mg, 0.0331 mmol). A premixed THPTA (3.5 mg) and sodium ascorbate (20 mg) in water (0.3 ml) was added, then copper(II)sulfate (2 mg). The ice water bath was removed, and the reaction mixture was stirred for 1 hour. It was then directly purified by reversed phase HPLC: 5 to 70% acetonitrile and water, using 0.1% TFA as modifier. Yield 20.2 mg, 63.7%. Ion(s) found by LCMS: [M+H]⁺=1207.2, [(M+2H)/2]⁺=604.2.

### Synthesis of Conjugate 54

To a solution of Fc SEQ ID NO: 13 (3.922 ml, 76.1 mg, 0.001307 mmol) in PBS 7.4 was added product from Int-102 (15 mg, 0.01046 mmol) in DMF (0.2 ml). Addition of DMF (0.2 ml×3) was used to wash the glassware and combined to the reaction mixture. The pH of the reaction mixture was slowly adjusted to ˜8.0 by 1M borate buffer solution (0.15 ml). The reaction was then gently rotate for 4 hours and then purified according to the general procedure. Maldi TOF analysis of the purified final product gave an average mass of 62,367 Da (DAR=3.9). Yield: 56.1 mg, 73.7%.

### Synthesis of Int-104

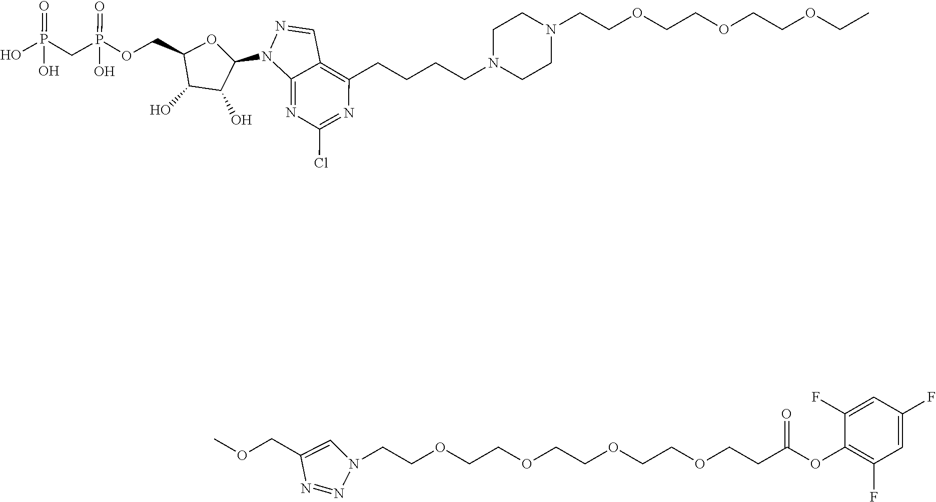

The title compound was prepared analogously to Int-102, where 1-((2-N-Boc-amino)ethyl)piperazine was replaced with (3-piperazin-1-yl-propyl)-carbamic acid tert-butyl ester. Ion(s) found by LCMS: [M+H]⁺=1221.2, [(M+2H)/2]⁺=610.2.

### Synthesis of Conjugate 55

The title compound was prepared analogously to Conjugate 54, where the product described in Int-102 was replaced with Int-104. Maldi TOF analysis of the purified final product gave an average mass of 62840 Da (DAR=4.3). Yield: 60.5 mg, 75.8%.

### Snthesis of Int-29

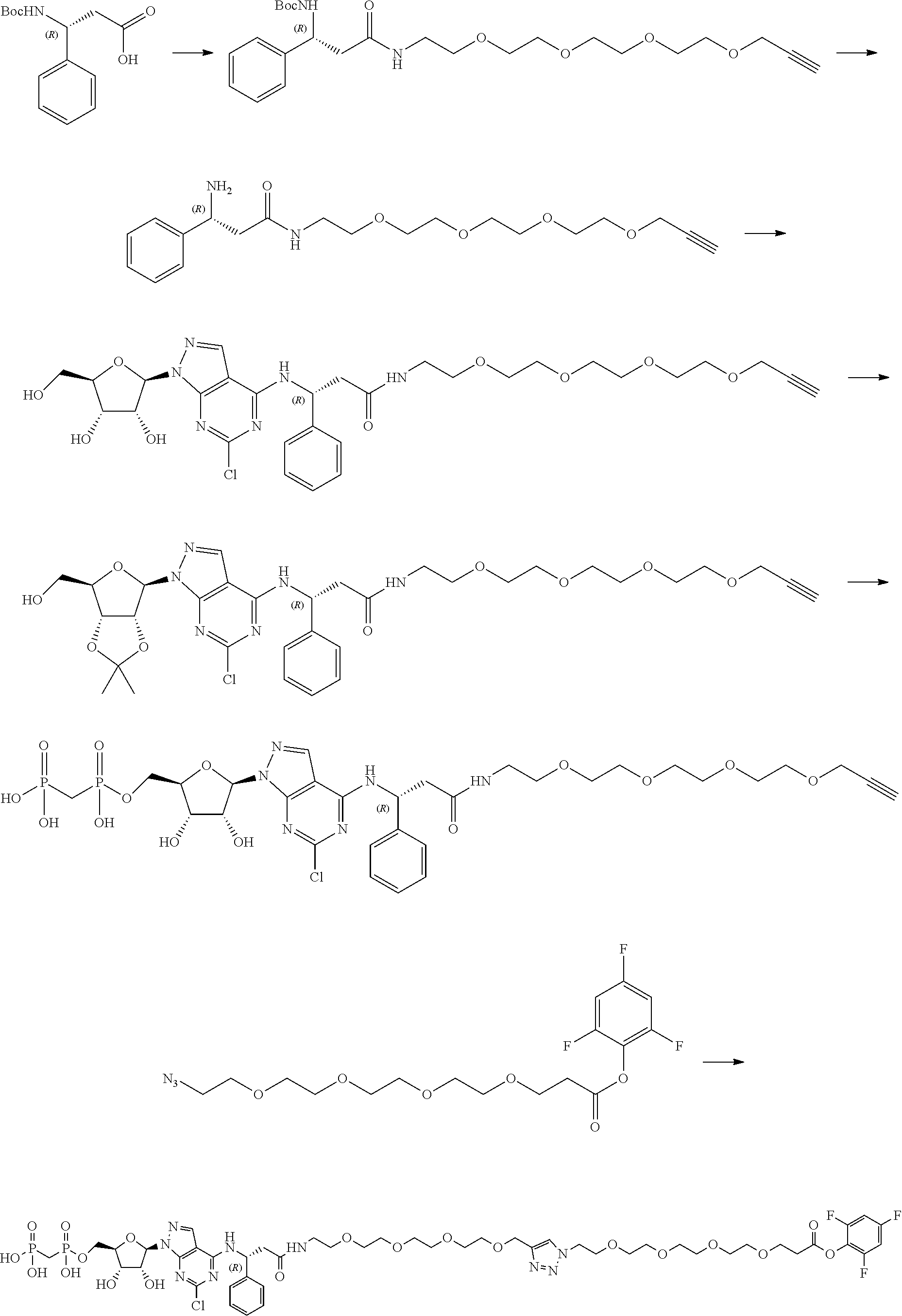

### Step a

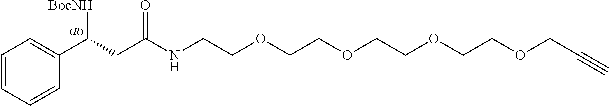

To a solution of (R)—N-Boc-3-amino-3-phenylpropanoic acid (1.0 g, 3.77 mmol) and propargyl-PEG4 amine (0.87 g, 3.77 mmol) in N,N-Dimethyl formamide (5 mL) was added HATU (2.87 g, 7.54 mmol) and DIEA (4.2 mL, 24.2 mmol). The reaction mixture was stirred at room temperature overnight, concentrated and purified by reversed phase HPLC (5% to 95% acetonitrile and water, using 0.1% TFA as modifier). Yield 1.2 g, 67%. Ion(s) found by LCMS: [M+H]+=479.2.

### Step b

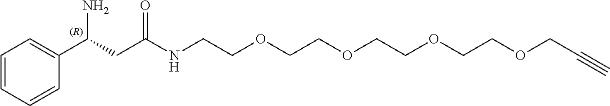

The product from the previous step (1.2 g, 2.6 mmol) was dissolved in a HCl solution (10 ml, 4N in dioxane) and reaction was stirred for 2 hours at ambient temperature. The solution was concentrated, and the crude product was carried to the subsequent step without any further purification. Yield of HCl salt 0.87 g. Ion(s) found by LCMS: [M+H]⁺=379.2

### Step c

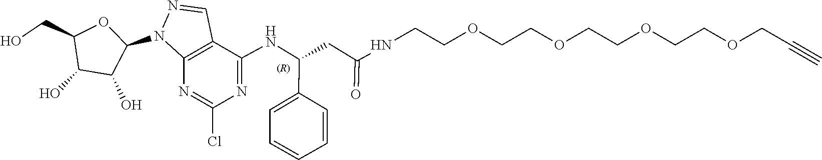

A solution of the product from the previous step (0.8 g, 2.11 mmol), intermediate B (1.13 g, 2.54 mmol), and triethylamine (0.44 mL, 3.17 mmol) in anhydrous ethanol (5 mL) was sealed in a screw-top vial and heated to 65° C. for 1 hour. After cooling to ambient temperature, 25% sodium methoxide in methanol (2.9 mL, 12.68 mmol) was added and the mixture was stirred for one hour. The reaction mixture was acidified with acetic acid (3 mL) then concentrated to dryness under reduced pressure and purified by silica gel chromatography (0→10% MeOH/DCM). Yield 1.12 g, 60%. Ion(s) found by LCMS: [M+H]⁺=662.8.

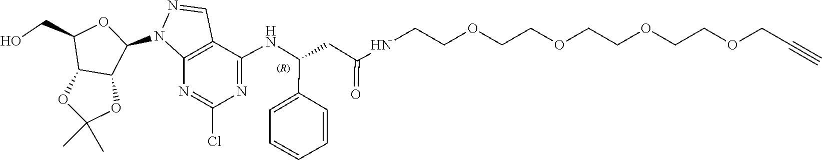

The product (1.12 g, 2.11 mmol) from the previous step was dissolved in acetone (5.0 mL). 2,2-dimethoxypropane (5.0 mL) and p-TsOH (0.218 g, 1.27 mmol) were added, and the mixture was stirred for 1 hour at ambient temperature. The reaction mixture was neutralized by adding diisopropyl ethyl amine (2 mL) and concentrated to dryness under reduced pressure and purified by silica gel chromatography (0→10% MeOH/DCM). Yield 0.98 g, 82.3%. Ion(s) found by LCMS: [M+H]+=703.2.

### Step d

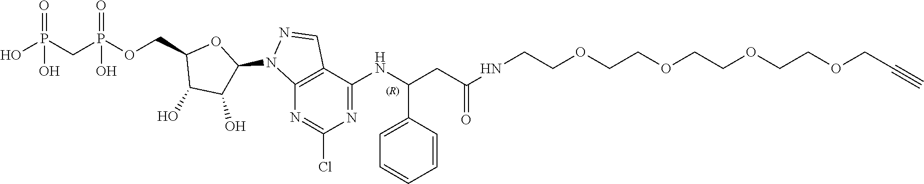

The product from the previous step (0.49 g, 0.70 mmol) in THE (2 mL) was added, dropwise, to a mixture of methylene (bis phosphonic dichloride) (0.52 g, 2.09 mmol) in THE (1 mL) and DIPEA (0.133 mL, 0.766 mmol) cooled to 0° C. via an ice bath. When the addition was complete the ice bath was removed, and the reaction was stirred for 3 hours at ambient temperature. The mixture was then cooled to 0° C. via an ice bath and 0.2 N aqueous HCl (6 mL) was added. The mixture was stirred at 0° C. for 15 minutes then at ambient temperature for 3 hours (monitored by LC/MS). The solvent was evaporated on the rotary evaporator and the crude mixture was purified by reversed phase HPLC (5-85% acetonitrile in DI water, 0.1% TFA modifier, 25 min gradient). The pure fractions were pooled and lyophilized to afford the product as a white, hygroscopic solid. Yield 0.14 g, 25%. Ion(s) found by LCMS: [M+H]+=821.2.

### Step e

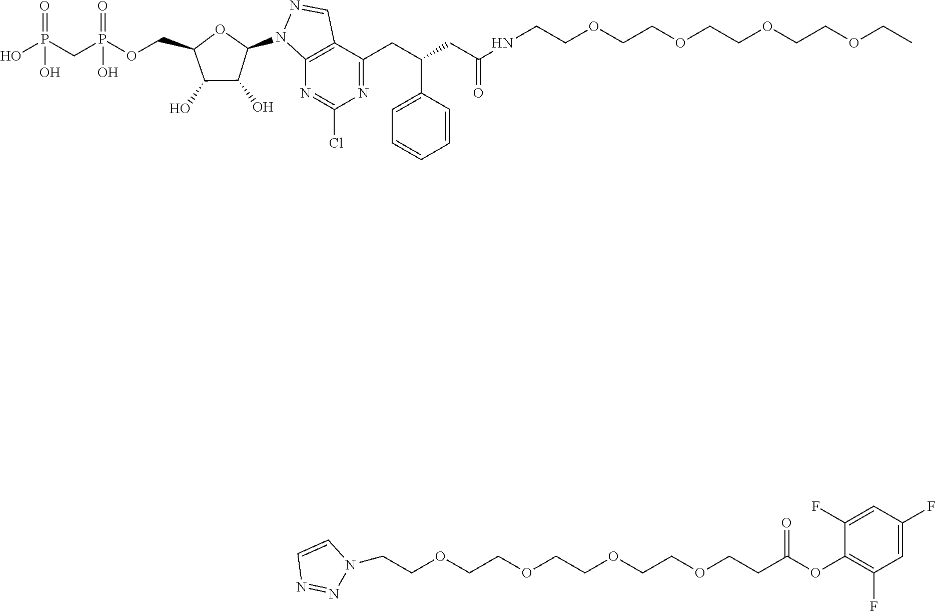 [SMILES: CCOCCOCCOCCOCCNC(=O)C[C@@H](Cc1nc(Cl)nc2c1cnn2[C@@H]1O[C@H](COP(=O)(O)CP(=O)(O)O)[C@@H](O)[C@H]1O)c1ccccc1 ; O=C(CCOCCOCCOCCOCCn1ccnn1)Oc1c(F)cc(F)cc1F]

The product from the previous step (42 mg, 0.05 mmol) and intermediate A (30.0 mg, 0.06 mmol) were dissolved in DMF (1 mL) and cooled to 0° C. via an ice water bath. Copper(II)sulfate (4.1 mg, 0.025 mmol) was added to a mixture of THPTA (11 mg, 0.025 mmol) and sodium ascorbate (10 mg, 0.05 mmol) in DI water (0.5 mL) and the mixture was gently shaken for 15-20 seconds until the solution was clear. The copper mixture was added to the alkyne/azido mixture and the reaction was stirred at 0° C. for 10 minutes then at ambient temperature for 20 minutes. The crude reaction mixture was purified by reversed phase HPLC (5-85% acetonitrile in DI water, 0.1% TFA modifier, 25 min gradient). The pure fractions were pooled and lyophilized to afford the product as a white, hygroscopic solid. Yield 46 mg, 72%. Ion(s) found by LCMS: [M+H)]⁺=1242.2 Synthesis of Conjugate 18 To a solution of Fc SEQ ID NO: 13 (2.55 ml, 50 mg, 0.0017 mmol) in PBS 7.4 was added Int-29 (17 mg, 0.0137 mmol) in DMF (0.200 ml). The pH of the reaction mixture was slowly adjusted to ˜8.5 with 1M borate buffer solution (0.6 ml). The reaction was then gently rotated overnight. The reaction mixture was quenched by adding a 150 mM Histidine/100 mM ammonium hydroxide buffer, pH 8.5, (˜2.5 mL of buffer mixture) and stirring for 12 hours then purified according to the general procedure. Maldi TOF analysis of the purified final product gave an average mass of 67,899 Da (DAR=8.8). Yield: 58 mg, 58%.

### Synthesis of Int-77

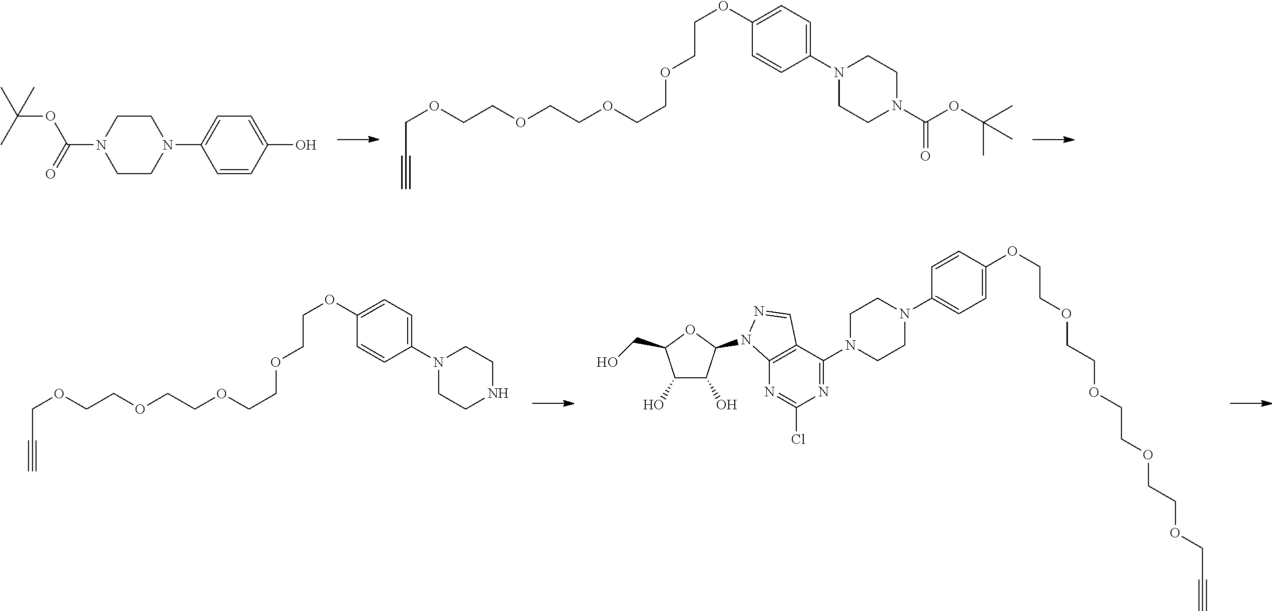 [SMILES: CC(C)(C)OC(=O)N1CCN(c2ccc(O)cc2)CC1 ; C#CCOCCOCCOCCOCCOc1ccc(N2CCN(C(=O)OC(C)(C)C)CC2)cc1 ; C#CCOCCOCCOCCOCCOc1ccc(N2CCNCC2)cc1 ; C#CCOCCOCCOCCOCCOc1ccc(N2CCN(c3nc(Cl)nc4c3cnn4[C@@H]3O[C@H](CO)[C@@H](O)[C@H]3O)CC2)cc1]

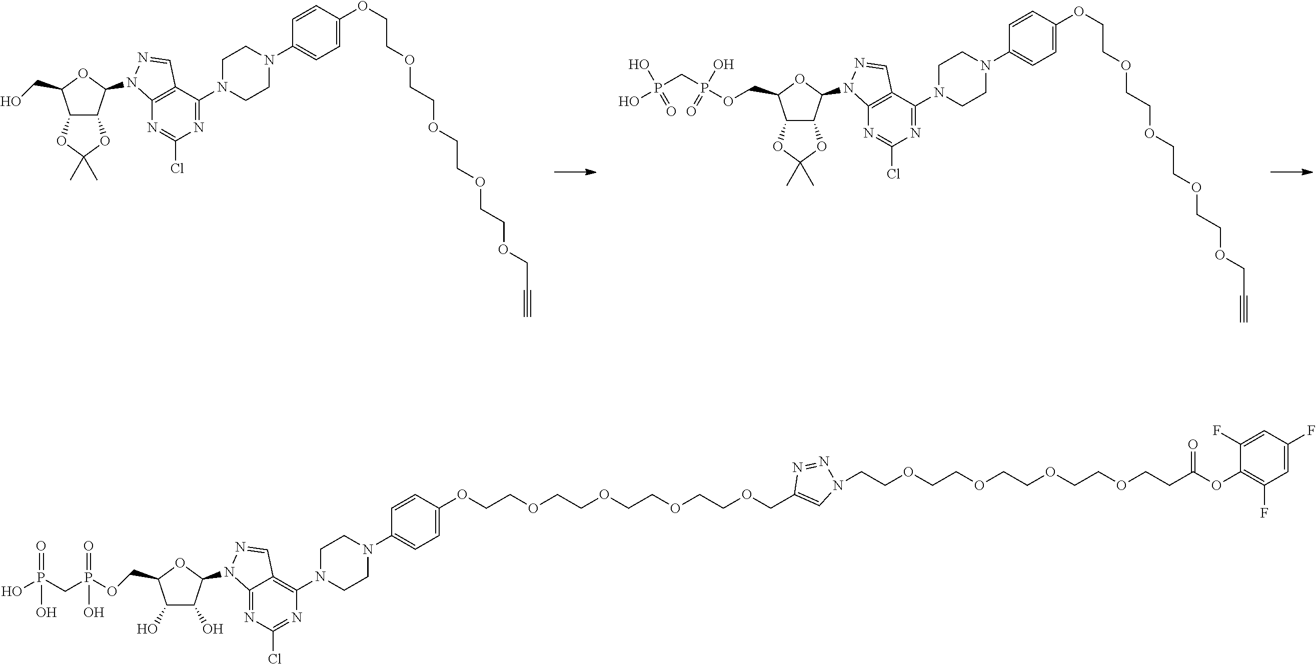 [SMILES: C#CCOCCOCCOCCOCCOc1ccc(N2CCN(c3nc(Cl)nc4c3cnn4[C@@H]3O[C@H](CO)[C@H]4OC(C)(C)O[C@H]43)CC2)cc1 ; C#CCOCCOCCOCCOCCOc1ccc(N2CCN(c3nc(Cl)nc4c3cnn4[C@@H]3O[C@H](COP(=O)(O)CP(=O)(O)O)[C@H]4OC(C)(C)O[C@H]43)CC2)cc1 ; O=C(CCOCCOCCOCCOCCn1cc(COCCOCCOCCOCCOc2ccc(N3CCN(c4nc(Cl)nc5c4cnn5[C@@H]4O[C@H](COP(=O)(O)CP(=O)(O)O)[C@@H](O)[C@H]4O)CC3)cc2)nn1)Oc1c(F)cc(F)cc1F]

### Step a

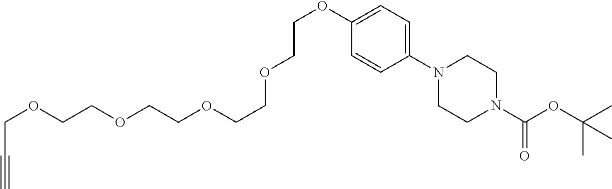 [SMILES: C#CCOCCOCCOCCOCCOc1ccc(N2CCN(C(=O)OC(C)(C)C)CC2)cc1]

To a solution of tert-butyl 4-(4-hydroxyphenyl)piperazine-1-carboxylate (1.0 g, 3.59 mmol) and propargyl-PEG4 mesylate (0.87 g, 3.59 mmol) in acetonitrile (5 mL) was added cesium carbonate (3.3 g, 10.15 mmol). The reaction mixture was stirred at 65° C. overnight, concentrated and purified by reversed phase HPLC (5% to 95% acetonitrile and water, using 0.1% TFA as modifier). Yield 1.29 g, 73%. Ion(s) found by LCMS: [M+H]⁺=493.2.

### Step b

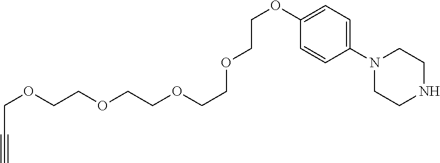 [SMILES: C#CCOCCOCCOCCOCCOc1ccc(N2CCNCC2)cc1]

The product from the previous step (1.29 g, 2.62 mmol) was dissolved in a HCl solution (10 ml, 4 N in dioxane) and reaction was stirred for 2 hours at ambient temperature. The solution was concentrated, and the crude product was carried to the subsequent step without any further purification. Ion(s) found by LCMS: [M+H]⁺=393.0

### Step c

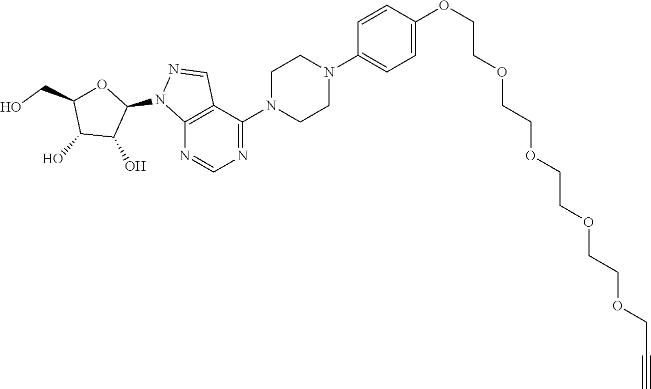 [SMILES: C#CCOCCOCCOCCOCCOc1ccc(N2CCN(c3ncnc4c3cnn4[C@@H]3O[C@H](CO)[C@@H](O)[C@H]3O)CC2)cc1]

A solution of the product from the previous step (0.75 g, 1.91 mmol), intermediate B (0.85 g, 1.91 mmol), and triethylamine (0.80 mL, 5.73 mmol) in anhydrous ethanol (5 mL) was sealed in a screw-top vial and heated to 65° C. for 1 hour. After cooling to ambient temperature, 25% sodium methoxide in methanol (2.62 mL, 11.47 mmol) was added and the mixture was stirred for one hour. The reaction mixture was concentrated acidified with acetic acid (3 mL) to dryness under reduced pressure and purified by silica gel chromatography (0→10% MeOH/DCM). Yield 0.76 g, 59%. Ion(s) found by LCMS: [M+H]⁺=676.6.

### Step d

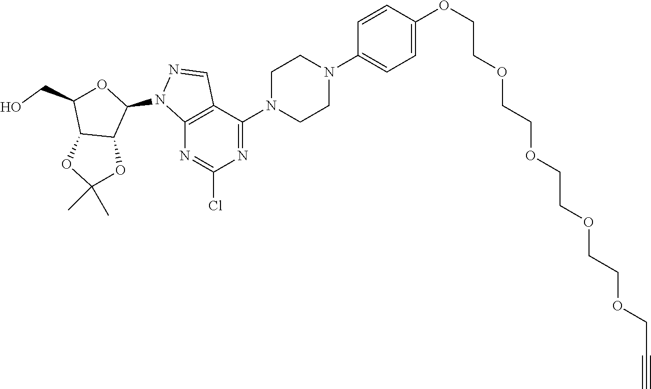 [SMILES: C#CCOCCOCCOCCOCCOc1ccc(N2CCN(c3nc(Cl)nc4c3cnn4[C@@H]3O[C@H](CO)[C@H]4OC(C)(C)O[C@H]43)CC2)cc1]

The product (1.0 g, 1.48 mmol) from the previous step was dissolved in acetone (5.0 mL). 2,2-dimethoxypropane (5.0 mL) and p-TsOH (0.218 g, 1.27 mmol) were added, and the mixture was stirred 1 hour at ambient temperature. The reaction mixture was neutralized by adding DIEA (2 mL) and concentrated to dryness under reduced pressure and purified by silica gel chromatography (0→10% MeOH/DCM). Yield 0.75 g, 71%. Ion(s) found by LCMS: [M+H]⁺=716.6.

### Step e

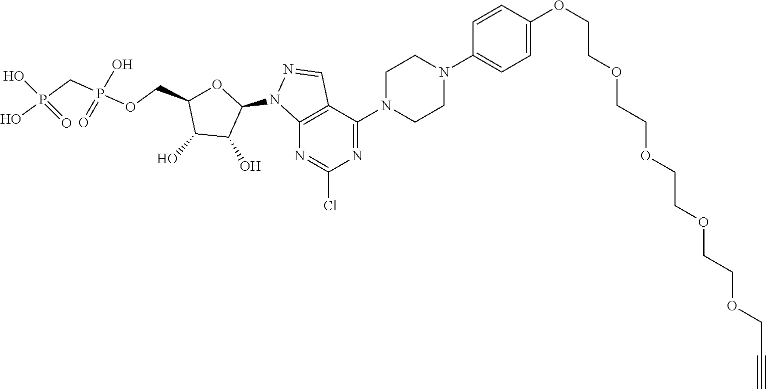 [SMILES: C#CCOCCOCCOCCOCCOc1ccc(N2CCN(c3nc(Cl)nc4c3cnn4[C@@H]3O[C@H](COP(=O)(O)CP(=O)(O)O)[C@@H](O)[C@H]3O)CC2)cc1]

To a solution of the product from the previous step (0.16 g, 0.70 mmol) in THE (1 mL) was added powdered methylene (bis phosphonic dichloride) (0.222 g, 0.89 mmol) followed by DIPEA (0.048 mL, 0.267 mmol). The reaction was stirred for 1.5 hours at ambient temperature. The mixture was then cooled to 0° C. via an ice bath, 1.5 mL water was added followed by 6 N aqueous HCl (0.5 mL). The mixture was stirred at 0° C. for 15 minutes then at ambient temperature for 1.5 hours (monitored by LC/MS). The solvent was evaporated on the rotary evaporator and the crude mixture was purified by reversed phase HPLC (5-85% acetonitrile in DI water, 0.1% TFA modifier, 25 min gradient). The pure fractions were pooled and lyophilized to afford the product as a white, hygroscopic solid. Yield 0.065 g, 35%. Ion(s) found by LCMS: [M+Na]⁺=859.4.

### Step f

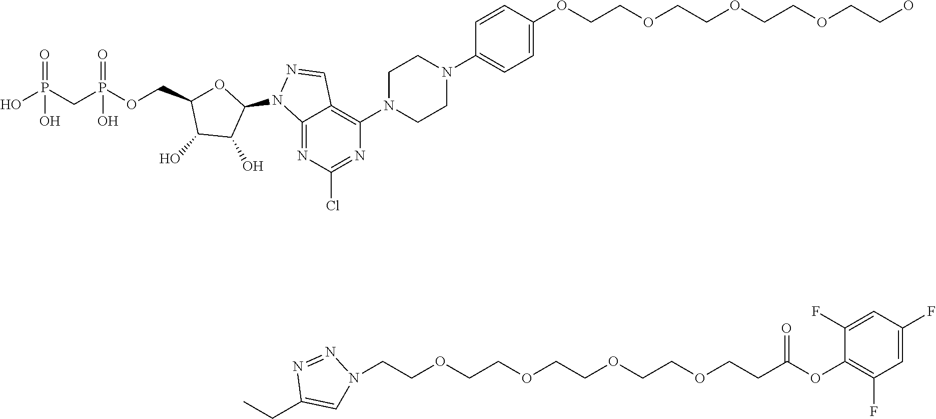 [SMILES: O=P(O)(O)CP(=O)(O)OC[C@H]1O[C@@H](n2ncc3c(N4CCN(c5ccc(OCCOCCOCCOCCO)cc5)CC4)nc(Cl)nc32)[C@H](O)[C@@H]1O ; CCc1cn(CCOCCOCCOCCOCCC(=O)Oc2c(F)cc(F)cc2F)nn1]

The product from the previous step (45 mg, 0.054 mmol) and intermediate A (27.3 mg, 0.065 mmol) were dissolved in DMF (1 mL) and cooled to 0° C. via an ice water bath. copper(II)sulfate (4.3 mg, 0.027 mmol) was added to a mixture of THPTA (12 mg, 0.027 mmol) and sodium ascorbate (11 mg, 0.054 mmol) in DI water (0.5 mL) and the mixture was gently shaken for 15-20 seconds until the solution was clear. The copper mixture was added to the alkyne/azido mixture and the reaction was stirred at 0° C. for 10 minutes then at ambient temperature for 20 minutes. The crude reaction mixture was purified directly by reversed phase HPLC (5-85% acetonitrile in DI water, 0.1% TFA modifier, 25 min gradient). The pure fractions were pooled and lyophilized to afford the product as a white, hygroscopic solid. Yield 43 mg, 64%. Ion(s) found by LCMS: [M+2H)/2]⁺=628.6

### Synthesis of Conjugate 42

To a solution of Fc SEQ ID NO: 13 (2.58 ml, 50 mg, 0.00086 mmol) in PBS 7.4 was added product from Int-77 (9.4 mg, 0.0069 mmol) in DMF (0.200 ml). The pH of the reaction mixture was slowly adjusted to ˜8.5 with 1M borate buffer solution (0.3 ml). The reaction was then rocked gently overnight. The reaction mixture was quenched by adding a 150 mM histidine/100 mM ammonium hydroxide buffer, pH 8.5, (˜2.5 mL of buffer mixture) and stirring for 12 hours then purified according to the general procedure. Maldi TOF analysis of the purified final product gave an average mass of 63,049 Da (DAR=4.4). Yield: 41.5 mg, 83%.

### Synthesis of Int-79

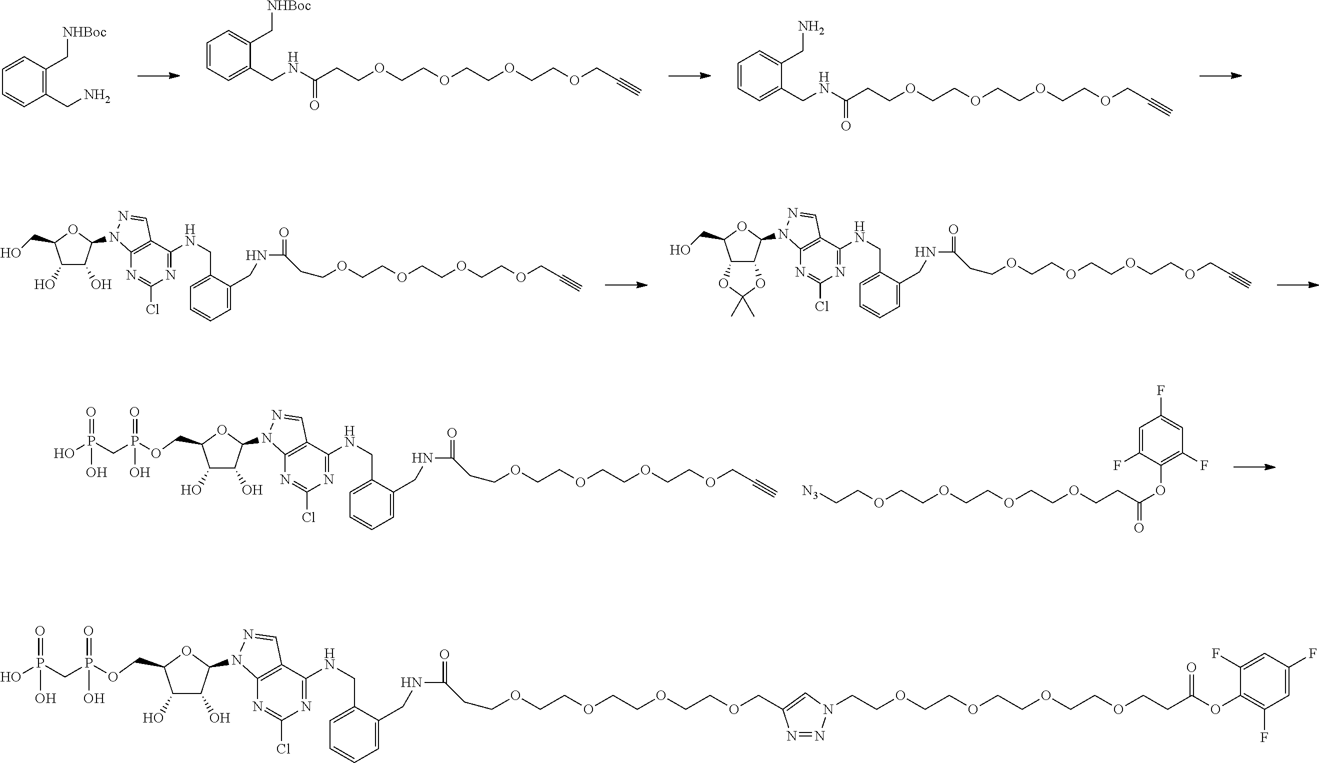

### Step a

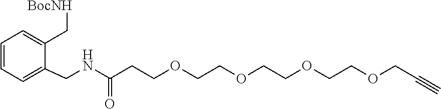 [SMILES: C#CCOCCOCCOCCOCCC(=O)NCc1ccccc1CNC(=O)OC(C)(C)C]

To a solution of propargyl-PEG4 carboxylic acid (1.10 g, 4.23 mmol) in DMF (5 mL) was added HATU (4.82 g, 12.70 mmol) and DIEA (4.7 mL, 25.39 mmol) then the reaction was stirred five minutes at ambient temperature. Mono-Boc protected 1,2 bis-methylamine 1,2 (1.0 g, 4.23 mmol) was added and the reaction mixture was stirred at ambient temperature overnight then concentrated and purified by reversed phase chromatography (5% to 95% acetonitrile and water, using 0.1% TFA as modifier). Yield 1.29 g, 64%. Ion(s) found by LCMS: [M+H]⁺=479.2.

### Step b

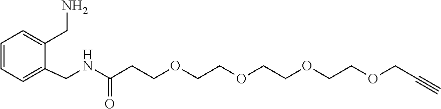 [SMILES: C#CCOCCOCCOCCOCCC(=O)NCc1ccccc1CN]

The product from the previous step (1.29 g, 2.70 mmol) was dissolved in a HCl solution (10 ml, 4 N in dioxane) and reaction was stirred for 2 hours at ambient temperature. The solution was concentrated, and the crude product was carried to the next step without any further purification. Yield of HCl salt 0.87 g. Ion(s) found by LCMS: [M+H]⁺=379.2

### Step c

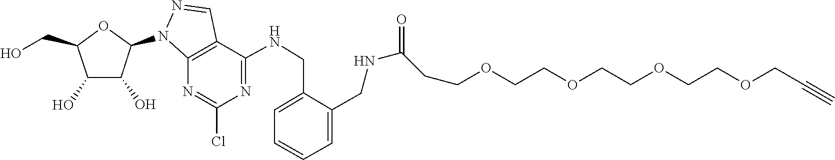 [SMILES: C#CCOCCOCCOCCOCCC(=O)NCc1ccccc1CNc1nc(Cl)nc2c1cnn2[C@@H]1O[C@H](CO)[C@@H](O)[C@H]1O]

A solution of the product from the previous step (0.5 g, 1.32 mmol), intermediate B (0.59 g, 1.32 mmol), and triethylamine (0.44 mL, 3.17 mmol) in anhydrous ethanol (5 mL) was sealed in a screw-top vial and heated to 65° C. for 1 h. After cooling to ambient temperature, 25% sodium methoxide in methanol (1.81 mL, 7.93 mmol) was added and the mixture was stirred for an hour. The reaction mixture was concentrated, then acidified with acetic acid (2 mL) and dried under reduced pressure, then purified by silica gel chromatography (0→10% MeOH/DCM). Yield 0.57 g, 65%. Ion(s) found by LCMS: [M+H]⁺=663.2.

### Step d

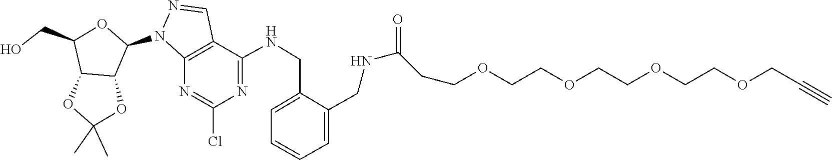 [SMILES: C#CCOCCOCCOCCOCCC(=O)NCc1ccccc1CNc1nc(Cl)nc2c1cnn2[C@@H]1O[C@H](CO)[C@H]2OC(C)(C)O[C@H]21]

The product (0.57 g, 0.86 mmol) from the previous step was dissolved in acetone (5.0 mL). 2,2-dimethoxypropane (5.0 mL) and p-TsOH (0.218 g, 1.27 mmol) were added, and the mixture was stirred for one hour at ambient temperature. The reaction mixture was neutralized by adding DIEA (2 mL) and concentrated to dryness under reduced pressure and purified by silica gel chromatography (0→10% MeOH/DCM). Yield 0.45 g, 75%. Ion(s) found by LCMS: [M+H]⁺=703.2.

### Step e

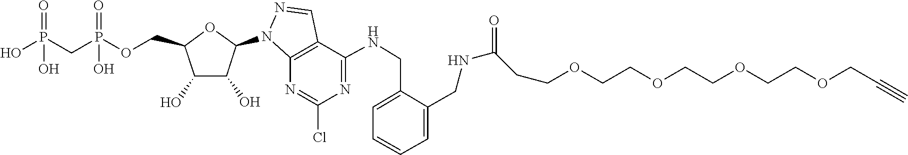 [SMILES: C#CCOCCOCCOCCOCCC(=O)NCc1ccccc1CNc1nc(Cl)nc2c1cnn2[C@@H]1O[C@H](COP(=O)(O)CP(=O)(O)O)[C@@H](O)[C@H]1O]

To a solution of the product from the previous step (0.5 g, 0.711 mmol) in THE (4 mL) was added methylene (bis phosphonic dichloride) (0.533 g, 2.133 mmol) followed by DIEA (0.107 mL, 0.782 mmol). The reaction was stirred for 1.5 hours at ambient temperature. The mixture was then cooled to 0° C. via an ice bath, 3 mL water was added followed by 6 N aqueous HCl (1.0 mL). The mixture was stirred at 0° C. for 15 minutes then at ambient temperature for 1.5 hours (monitored by LC/MS). The solvent was evaporated on the rotary evaporator and the crude mixture was purified by reversed phase HPLC (5-85% acetonitrile in DI water, 0.1% TFA modifier, 25 min gradient). The pure fractions were pooled and lyophilized to afford the product as a white, hygroscopic solid. Yield 0.16 g, 26%. Ion(s) found by LCMS: [M+H]⁺=821.2.

### Step f

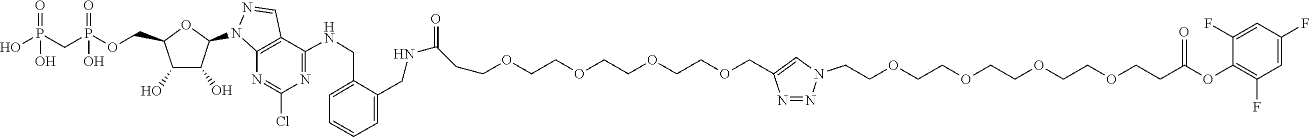 [SMILES: O=C(CCOCCOCCOCCOCc1cn(CCOCCOCCOCCOCCC(=O)Oc2c(F)cc(F)cc2F)nn1)NCc1ccccc1CNc1nc(Cl)nc2c1cnn2[C@@H]1O[C@H](COP(=O)(O)CP(=O)(O)O)[C@@H](O)[C@H]1O]

The product from the previous step (55 mg, 0.067 mmol) and intermediate A (34 mg, 0.08 mmol) were dissolved in DMF (1 mL) and cooled to 0° C. via an ice water bath. Copper(II)sulfate (5.2 mg, 0.033 mmol) was added to a mixture of THPTA (14.5 mg, 0.033 mmol) and sodium ascorbate (10 mg, 0.067 mmol) in DI water (0.5 mL) and the mixture was gently shaken for 15-20 seconds until the solution was clear. The copper mixture was added the alkyne/azido mixture and the reaction was stirred at 0° C. for 10 minutes then at ambient temperature for 20 minutes. The crude reaction mixture was purified directly by reversed phase HPLC (5-85% acetonitrile in DI water, 0.1% TFA modifier, 25 min gradient). The pure fractions were pooled and lyophilized to afford the product as a white, hygroscopic solid. Yield 56 mg, 67%. Ion(s) found by LCMS: [M+2H)/2]⁺=621.2 Synthesis of Conjugate 43 To a solution of Fc SEQ ID NO: 13 (5.15 ml, 100 mg, 0.0017 mmol) in PBS 7.4 was added product from Int-79 (17 mg, 0.013 mmol) in DMF (0.200 ml). The pH of the reaction mixture was slowly adjusted to ˜8.5 with 1M borate buffer solution (0.3 ml). The reaction was then gently rotate overnight. The reaction mixture was quenched by adding a 150 mM histidine/100 mM ammonium hydroxide buffer, pH 8.5, (˜2.5 mL of buffer mixture) and stirring for 12 hours then purified according to the general procedure. Maldi TOF analysis of the purified final product gave an average mass of 62,431 Da (DAR=3.8). Yield: 82.8 mg, 82.8%.

### Synthesis of Int-121

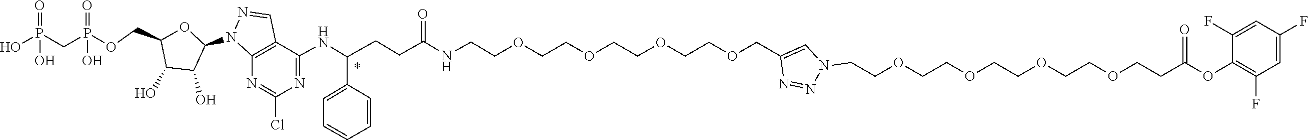 [SMILES: O=C(CCC(Nc1nc(Cl)nc2c1cnn2[C@@H]1O[C@H](COP(=O)(O)CP(=O)(O)O)[C@@H](O)[C@H]1O)c1ccccc1)NCCOCCOCCOCCOCc1cn(CCOCCOCCOCCOCCC(=O)Oc2c(F)cc(F)cc2F)nn1]

The title compound was prepared analogously to Int-29, Ions found by LCMS: [M+2H)/2]⁺=628.8.

### Synthesis of Conjugate 64

To a solution of Fc SEQ ID NO: 13 (2.58 ml, 50 mg, 0.00085 mmol) in PBS 7.4 was added product from Int-121 (17 mg, 0.0137 mmol) in DMF (0.200 ml). The pH of the reaction mixture was slowly adjusted to ˜8.5 by 1M potassium carbonate buffer solution (0.6 m1, pH 9). The reaction was then gently rotated overnight. The reaction mixture was quenched by adding a 150 mM histidine/100 mM ammonium hydroxide buffer, pH 8.5, (˜2.5 mL of buffer mixture) and stirring for 12 hours then purified according to the general procedure. Maldi TOF analysis of the purified final product gave an average mass 59176 Da (DAR=0.9). Yield: 31.1 mg, 62.2%.

### Synthesis of Int-123

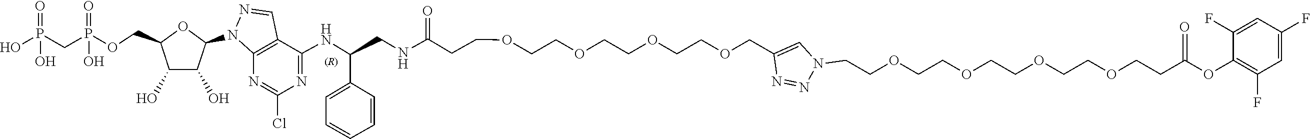

The title compound was prepared analogously to Int-79, Ions found by LCMS: [M+2H)/2]⁺=621.8.

### Synthesis of Conjugate 65

The title compound was prepared analogously to Conjugate 43, where product described in Int-79 was replaced with Int-123. Maldi TOF analysis of the purified final product gave an average mass of 62384 Da (DAR=3.8). Yield: 51.1 mg, 102.2%.

### Synthesis of Int-125

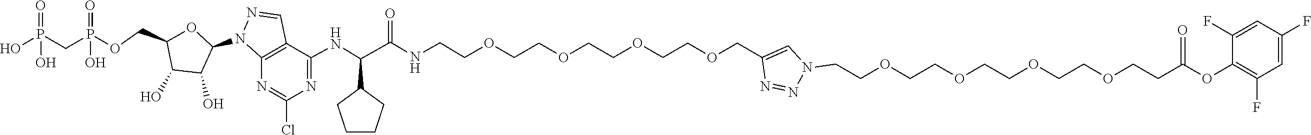 [SMILES: O=C(CCOCCOCCOCCOCCn1cc(COCCOCCOCCOCCNC(=O)[C@H](Nc2nc(Cl)nc3c2cnn3[C@@H]2O[C@H](COP(=O)(O)CP(=O)(O)O)[C@@H](O)[C@H]2O)C2CCCC2)nn1)Oc1c(F)cc(F)cc1F]

The title compound was prepared analogously to Int-129. Ions found by LCMS: [M+H]⁺=1220.2.

### Synthesis of Conjugate 66

The title compound was prepared analogously to Conjugate 18, where the product from Int-29 was replaced with Int-125. Maldi TOF analysis of the purified final product gave an average mass of 62,237 Da (DAR=3.9). Yield: 36 mg, 72%.

### Synthesis of N-[2-(2-{2-[2-(2-azidoethoxy)ethoxy]ethoxy}ethoxy)ethyl](4-nitrophenoxy)carboxamide

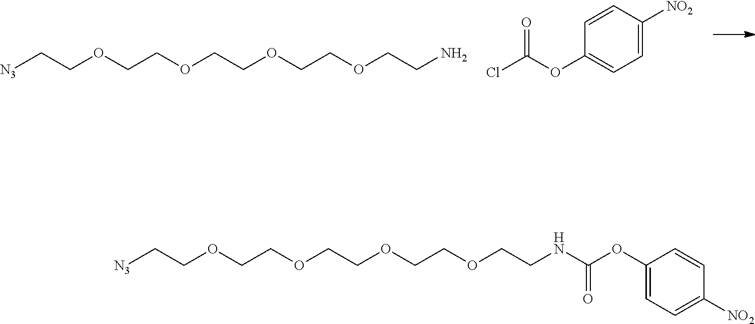 [SMILES: [N-]=[N+]=NCCOCCOCCOCCOCCN ; O=C(Cl)Oc1ccc([N+](=O)[O-])cc1 ; [N-]=[N+]=NCCOCCOCCOCCOCCNC(=O)Oc1ccc([N+](=O)[O-])cc1]

To a solution of azidoPEG₄ amine (500 mg, 1.91 mmol) and triethylamine (0.39 mL, 2.89 mmol) in DCM (25 mL) was added a solution of 4-nitrophenyl chloroformate (461 mg, 2.29 mmol) in DCM (5 mL). The resultant mixture was stirred at room temperature for 30 minutes Reaction mixture was diluted with DCM (50 mL) and washed with water and dried over sodium sulfate and evaporated under reduced pressure to get crude compound which was purified by silica gel column chromatography (Hexanes: EtOAc). Clear viscous oil. Yield 537 mg, 65.9%. Ion(s) found buy LCMS: [M+H]⁺=428.2.

### Synthesis of [bis(tert-butoxy)carbonyl]methyl (trifluoromethyl)sulfonate

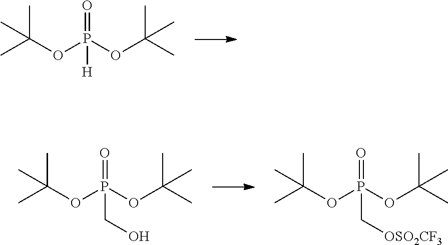

### Step a

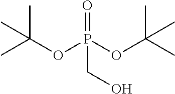

Di-tert-butyl phosphite (5.3 g, 2.75 mmol), paraformaldehyde (908 mg, 30.25 mmol) and potassium carbonate (760 mg, 5.5 mmol) in MeCN (HPLC grade, 85 mL) was purged with nitrogen and the colorless suspension was heated at 70° C. for 12 h under nitrogen atmosphere. The reaction mixture was cooled to r.t., filtered, and concentrated in vacuo at 40° C. to half of the original volume. The flask was cooled at −78° C. and sonicated. The supernatant mother liquor was decanted, and the remaining solid was washed twice with cold MeCN. The solid was dried in vacuo to give the desired product as a white solid. Yield 3.83 g, 62.11%. Ion(s) found buy LCMS: [M+H]⁺=225.2.

### Step b

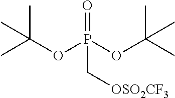

Hydroxymethyl phosphonic acid di-t-butyl ester from previous step (3.1 g, 13.83 mmol) was dissolved in 50 mL CH₂Cl₂ under argon. 2,6-lutidine (3.2 mL, 27.65 mmol) was added at −78° C. in one portion. Triflic anhydride (2.57 mL, 15.21 mmol) was added dropwise over 10 minutes. The reaction was stirred for 3 hours at −78° C. The reaction mixture was quenched with of water at 0° C., and organic layer collected, aqueous layer extracted with DCM twice, combined organic extracts were washed with 10% NaHCO₃ solution, brine and dried over sodium sulfate and removed solvent and vacuum dried to get the crude product, which was used without further purification. Reddish oil. 4.3 g, 87.29%. Ion(s) found buy LCMS: [M+Na]⁺=379.4.

### Synthesis of Int-255

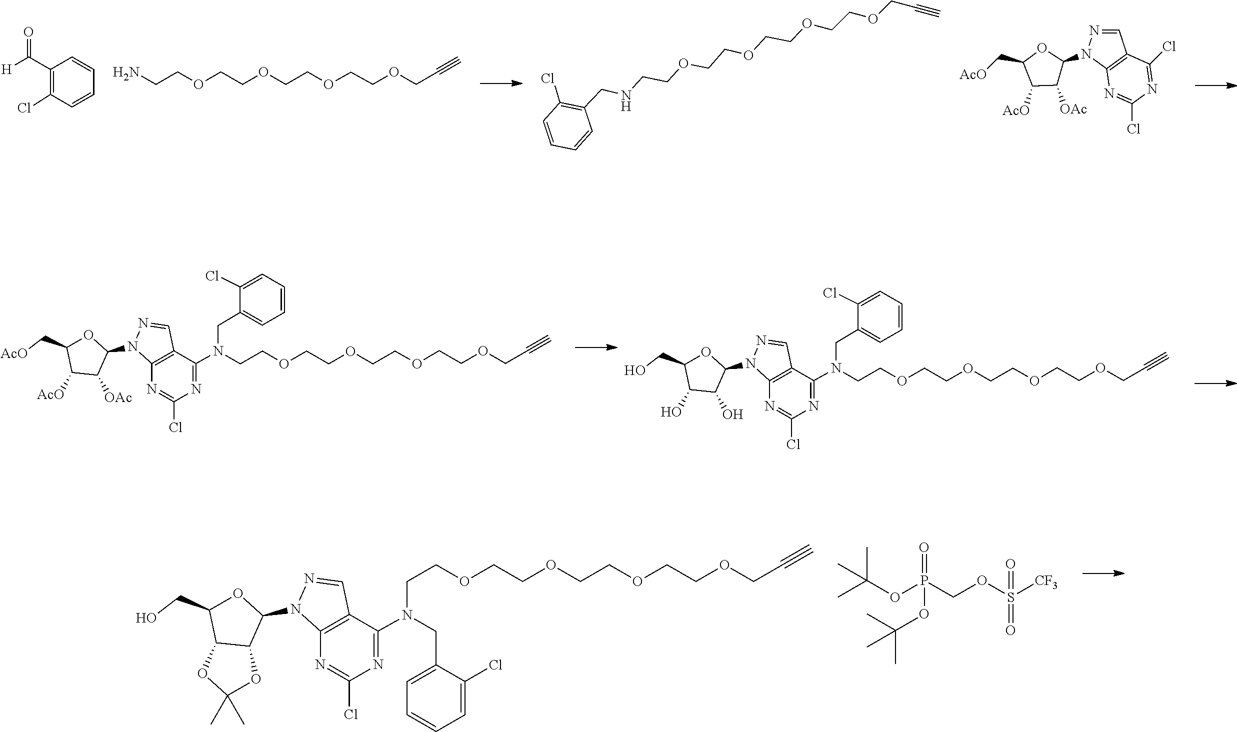

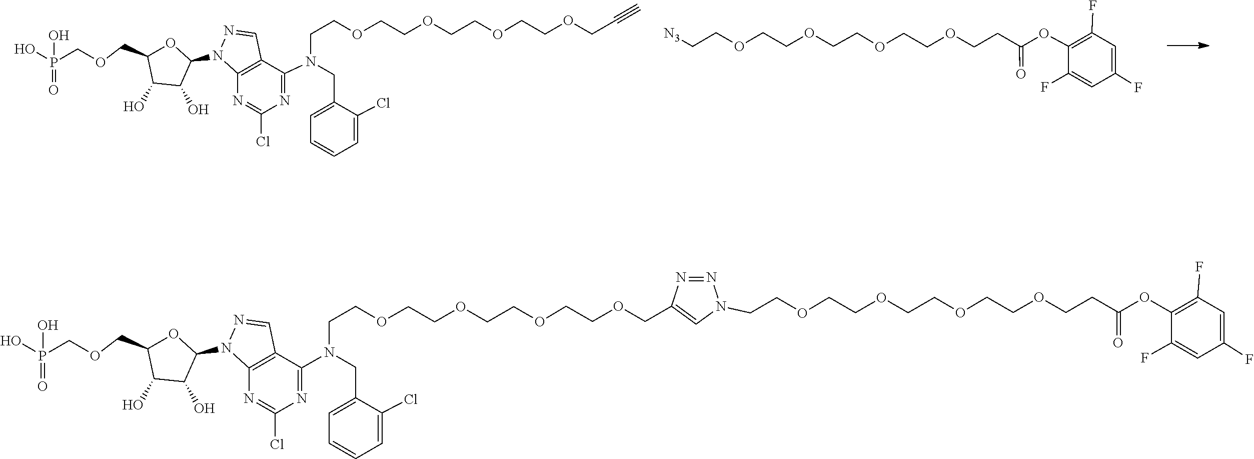

### Step a

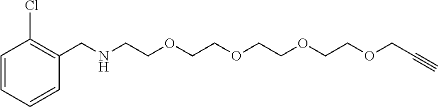 [SMILES: C#CCOCCOCCOCCOCCNCc1ccccc1Cl]

Propargyl-peg₄-amine (900 mg, 3.9 mmol) and 2-chloro-benzaldehyde (548 mg, 1.95 mmol) were stirred in methanol (10 mL) for 16 hours at ambient temperature. To this was added sodium borohydride (592 mg, 15.6 mmol) and the solution was stirred for an additional 1 hour. The excess methanol was removed by the rotary evaporator and the solution was partitioned between 1 N NaOH (50 mL) and ethyl acetate (50 mL). The aqueous layer was discarded, and the excess ethyl acetate was removed and the crude mixture was purified by reversed phase flash chromatography (0% to 100% CH₃CN/H₂O). Yield: 692 mg, 50%. Ion found by LCMS: [M+H]⁺=356.2.

### Step b

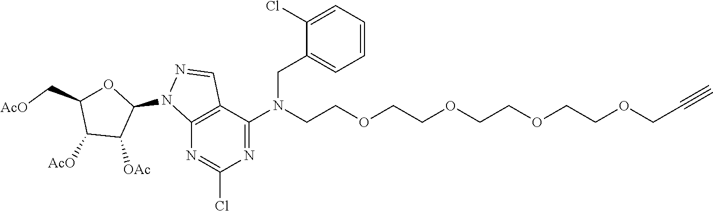 [SMILES: C#CCOCCOCCOCCOCCN(Cc1ccccc1Cl)c1nc(Cl)nc2c1cnn2[C@@H]1O[C@H](COC(C)=O)[C@@H](OC(C)=O)[C@H]1OC(C)=O]

Intermediate B (1 g, 2.23 mmol), the product from the previous step (568 mg, 2.45 mmol) and DIEA (0.77 mL, 4.47 mmol) were stirred in methanol (25 mL) at 50° C. for 1 h. The mixture was cooled to ambient temperature and concentrated. The crude residue was dissolved in EtOAc (50 mL) and washed with water and brine. The organic layer was dried over sodium sulfate and solvent was removed under reduced pressure to yield triacetate derivative which was purified by silica gel column chromatography (DCM to 10% MeOH in DCM) to yield the desired product as an off-white foam. Yield 1.2 g, 83.8%. Ion(s) found by LCMS: [M+H]⁺=766.2.

### Step c

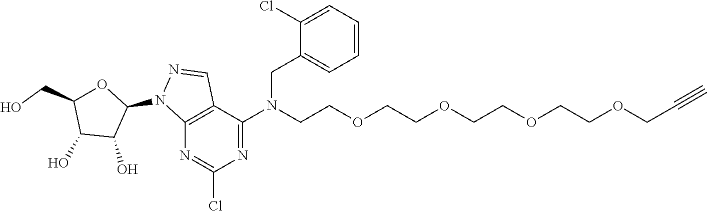 [SMILES: C#CCOCCOCCOCCOCCN(Cc1ccccc1Cl)c1nc(Cl)nc2c1cnn2[C@@H]1O[C@H](CO)[C@@H](O)[C@H]1O]

To a solution of the triacetate derivative (1.2 g, 1.57 mmol) in MeOH (25 mL) was added solid potassium carbonate (758 mg, 5.5 mmol) and the resulting mixture was stirred at room temperature for 2 h. The reaction mixture was concentrated and the residue was dissolved in EtOAc (50 mL) and washed with water and brine. The organic layer was dried over sodium sulfate and solvent was removed under reduced pressure to yield the product which was further purified by silica gel column chromatography (DCM to 10% MeOH in DCM) to yield the desired product as an off white solid. Yield 880 mg, 87.6%. Ion(s) found by LCMS: [M+H]⁺=640.2.

### Step d

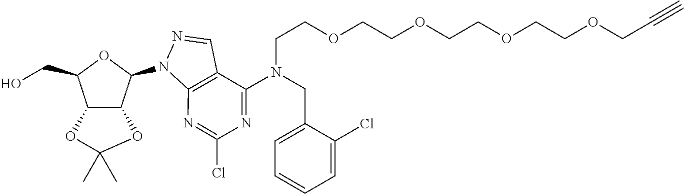 [SMILES: C#CCOCCOCCOCCOCCN(Cc1ccccc1Cl)c1nc(Cl)nc2c1cnn2[C@@H]1O[C@H](CO)[C@H]2OC(C)(C)O[C@H]21]

To a solution of the triol product from step above (880 mg, 1.37 mmol) and 2,2-dimethoxypropane (715 mg, 6.87 mmol) in acetone (50 mL) at room temperature was added p-TsOH (236 mg, 1.37 mmol).

The reaction was stirred for two hours then concentrated under reduced pressure. The crude oil was dissolved in EtOAc (50 mL) and washed with saturated NaHCO₃. The organic layer was separated and dried over sodium sulfate, filtered, and concentrated in vacuo to provide an off-white solid, which was used in the next step without further purification. Yield 890 mg, 95.18%. Ion(s) found by LCMS: [M+H]⁺=680.2.

### Step e

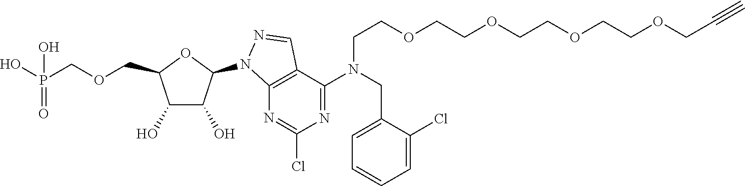 [SMILES: C#CCOCCOCCOCCOCCN(Cc1ccccc1Cl)c1nc(Cl)nc2c1cnn2[C@@H]1O[C@H](COCP(=O)(O)O)[C@@H](O)[C@H]1O]

A solution of acetonide derivative from previous step (130 mg, 0.191 mmol) and [bis(tert-butoxy)carbonyl]methyl (trifluoromethyl)sulfonate (136 mg, 0.38 mmol) in dry DMF (3 mL) was cooled using ice-water bath. 60% NaH in mineral oil (46 mg, 1.15 mmol) was added to this and gradually warmed to room temperature stirred until LC-MS showed completion of the reaction (˜2h). The reaction mixture was quenched with saturated ammonium chloride and extracted with EtOAc. The organic phase was washed with brine and dried over sodium sulfate concentrated to give crude product which was used in the next step without purification. Observed LCMS: 835.2 Crude product was dissolved in 50% aqueous TFA (10 mL) and stirred at room temperature for 2 h, concentrated and the residue was purified by reverse phase (10% to 100% ACN/water, 0.1% TFA modifier). The product was a white solid. Yield 89 mg, 63.43%. Ion(s) found by LCMS: [M+H]+=734.2.

### Step f

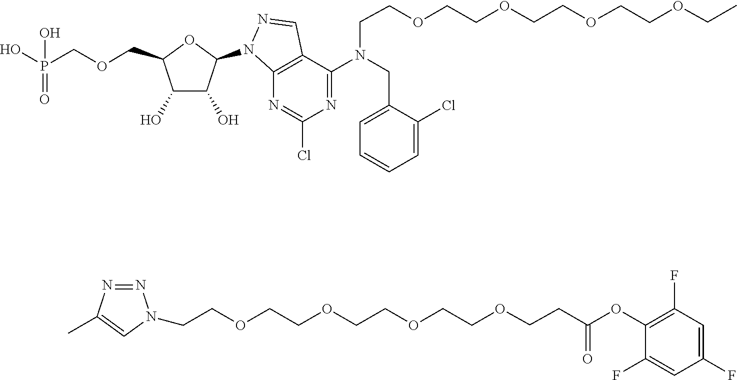

To a solution of the product from the previous step (40 mg, 0.054 mmol) and intermediate A (23 mg, 0.054 mmol), dissolved in DMF:H₂O (1:3, 1.5 mL) were cooled to 0° C. To this was added a pre-mixed solution of a solution of copper (II) sulfate (1 mg, 0.005 mmol), sodium ascorbate (32 mg, 0.163 mmol), and BTTA (5 mg, 0.011 mmol) dissolved in water (0.5 mL), then stirred for 5 minutes at the same temperature and gradually warmed to room temperature and stirred room temperature for 15 minutes.

When the reaction was complete (by HPLC), the mixture was quenched with a few drops of glacial acetic acid and 125 mM EDTA (pH 6). The crude product was purified by reversed phase HPLC (10% to 100% ACN/water, 0.1% TFA modifier). The product was a solid. Yield 43 mg, 68.31%. Ion(s) found by LCMS [(M+2H)/2]⁺=578.2.

### Synthesis of Conjugate 131

Trifluorophenol ester (16 mg, 0.014 mmol, Int-255) in DMF (0.5 mL) was added to Fc carrier SEQ ID NO: 13 (100 mg, 2.78 mL in PBS at pH 7.4) then adjusted to pH-7.4 with sodium carbonate buffer.

The mixture was rocked at ambient temperature for 4 hours. The reaction mixture was quenched by adding a 150 mM histidine/100 mM ammonium hydroxide buffer, pH 8.5, (˜0.5 mL of buffer mixture/10 mg of protein) and stirring for 12 hours, then purified by the general procedure. Maldi TOF analysis of the purified final product gave an average mass of 62,072 Da. (DAR=3.9). Yield 46.5 mg, 46.5%.

### Synthesis of Int-256

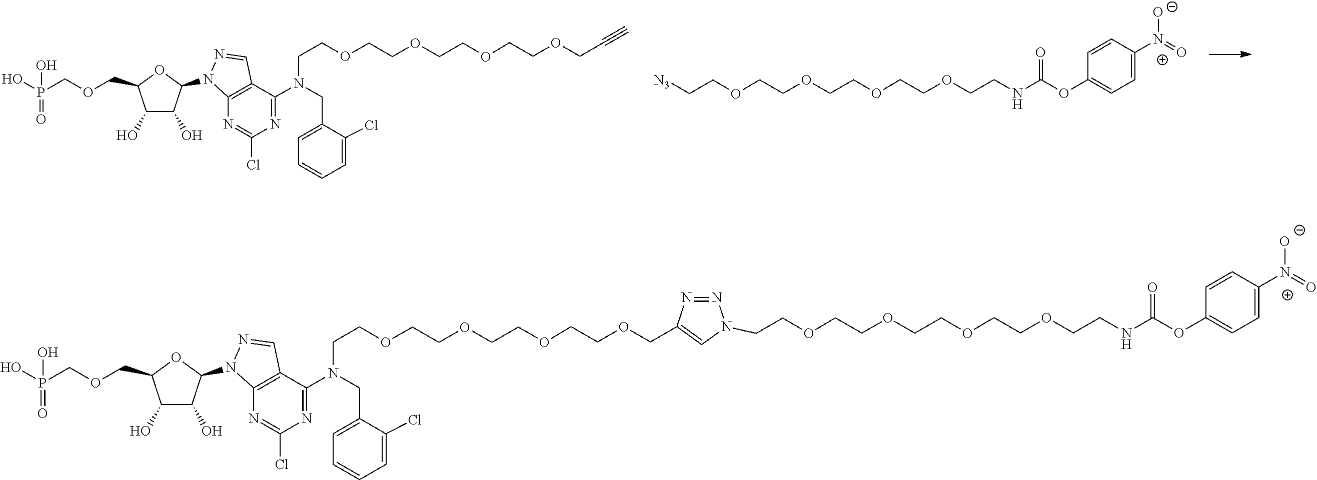

To a solution of product from step d of the synthesis of Int-255 (30 mg, 0.041 mmol) and N-[2-(2-{2-[2-(2-azidoethoxy)ethoxy]ethoxy}ethoxy)ethyl](4-nitrophenoxy)carboxamide and intermediate A (17 mg, 0.041 mmol) in DMF:H₂O (1:3, 1.5 mL) were cooled to 0° C. To this was added a pre-mixed solution of a solution of copper(II) sulfate (0.6 mg, 0.00041 mmol), sodium ascorbate (24 mg, 0.122 mmol), and BTTA (3.5 mg, 0.0085 mmol) dissolved in water (0.5 mL). The reaction was stirred for 5 minutes at the same temperature and then gradually warmed to room temperature and stirred room temperature for 15 minutes. When the reaction was complete (by HPLC), the mixture was quenched with a few drops of glacial acetic acid and 125 mM EDTA (pH 6), and the product was purified by reversed phase HPLC (10% to 100% ACN/water, 0.1% TFA modifier). The product was a white solid. Yield 28 mg, 59%. Ion(s) found by LCMS [M+H]⁺=1161.2.

### Synthesis of Conjugate 132

4-Nitrophenyl carbamate derivative from above step (11 mg, 0.009 mmol, Int-256) in DMF (0.5 mL) was added to Fc carrier SEQ ID NO: 80 (60 mg in 2.78 mL PBS at pH 7.4) as described in the conjugation procedure for Conjugate 131. Maldi TOF analysis of the purified final product gave an average mass of 62,199 Da. (DAR=6.9). Yield 27.3 mg, 45.4%.

### Synthesis of Int-279

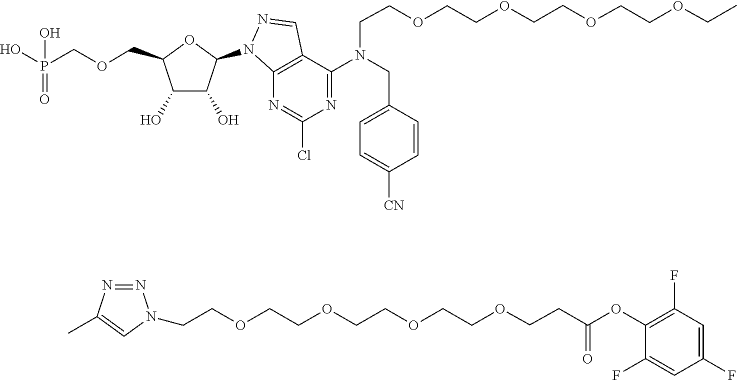

The title compound was prepared analogously to Int-255 where 2-chlorobenzaldehyde was replaced with 4-cyanobenzaldehyde. The product was a solid. Ion(s) found by LCMS [(M+2H)/2]⁺=573.8.

### Synthesis of Conjugate 147

Int-279 (13 mg, 0.0116 mmol) in DMF (0.5 mL) was added to SEQ ID NO: 80 (80 mg, 5.26 mL in PBS at pH 8.4) as described in the general conjugation procedure. Maldi TOF analysis of the purified final product gave an average mass of 56,338 Da. (DAR=1.1). Yield 32 mg, 40%.

### Synthesis of Int-280

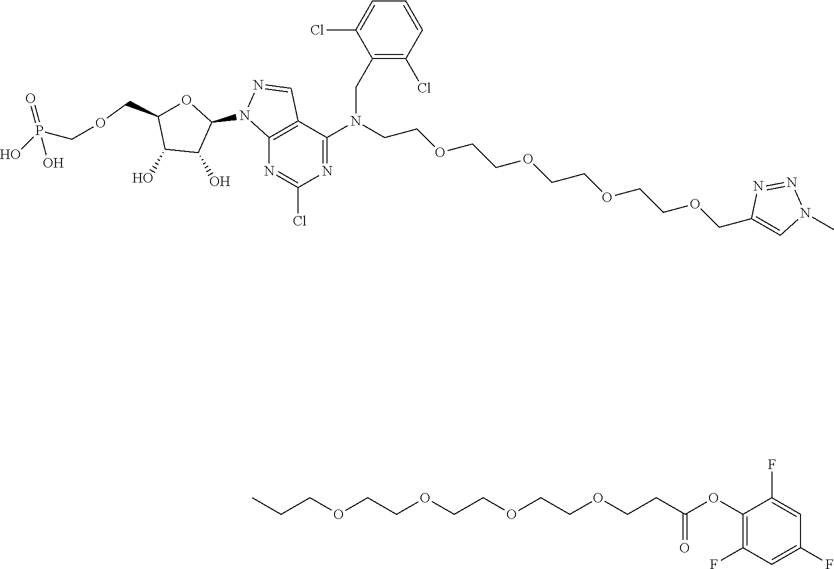

The title compound was prepared analogously to Int-255 where 2-chlorobenzaldehyde was replaced with 2,6-dichlorobenzaldehyde. The product was a white solid. Ion(s) found by LCMS: [(M+2H)/2]⁺=596.2.

### Synthesis of Conjugate 148

Int-280 (13.7 mg, 0.0116 mmol) was added to SEQ ID NO: 80 (80 mg, 5.26 mL in PBS at pH 8.4) as described in the general conjugation procedure. Maldi TOF analysis of the purified final product gave an average mass of 56,958 Da. (DAR=1.7). Yield 40 mg, 50%.

### Synthesis of Int-313

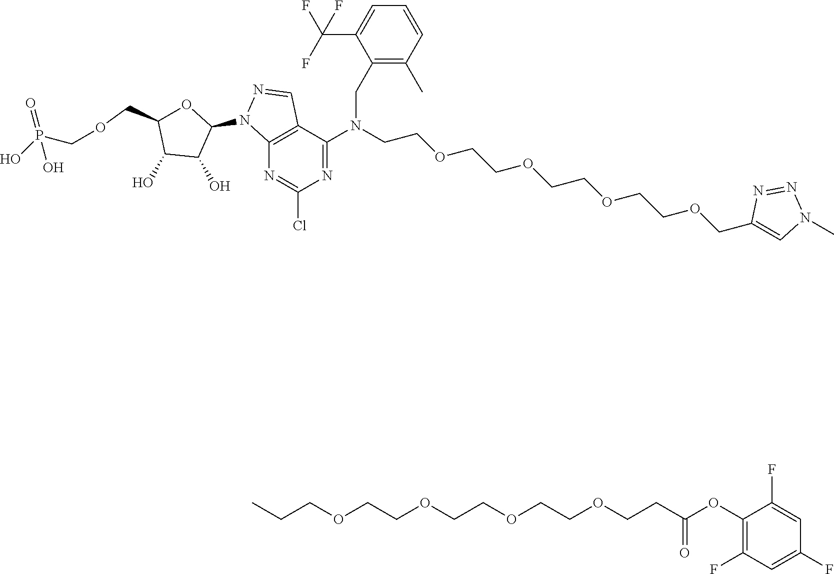

The title compound was prepared analogously to Int-255 where 2-chlorobenzaldehyde was replaced with 6-methyl-2-(trifluoromethyl)benzaldehyde. The product was a solid. Ion(s) found by LCMS [(M+2H)/2]⁺=614.2.

### Synthesis of Conjugate 180

Int-313 (18 mg, 0.0165 mmol) in DMF (0.5 mL) was added to SEQ ID NO: 80 (100 mg, 7.75 mL in PBS at pH 8.4) as described in the general conjugation procedure. Maldi TOF analysis of the purified final product gave an average mass of 61,479 Da. (DAR=5.9). Yield 62 mg, 51%.

### Synthesis of Int-317

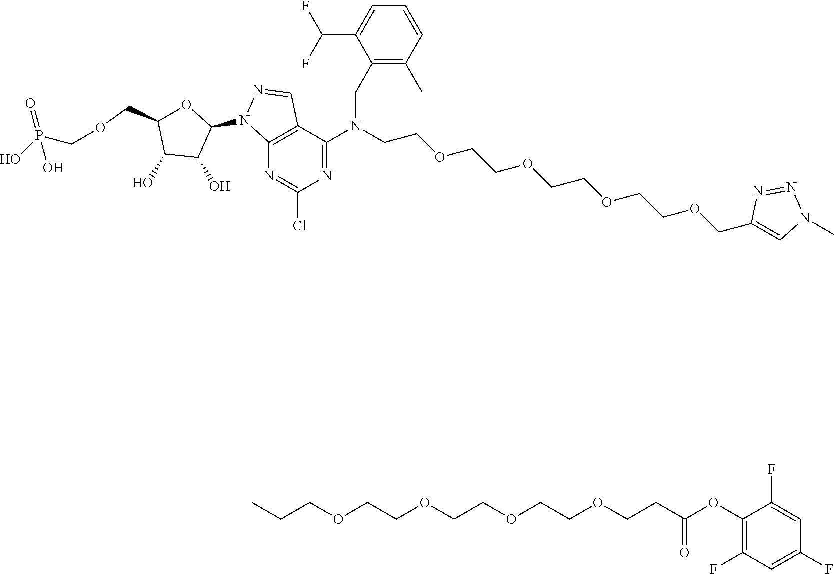

The title compound was prepared analogously to Int-255 where 2-chlorobenzaldehyde was replaced with 2-(difluoromethyl)benzaldehyde. The product was a solid. Ion(s) found by LCMS [(M+2H)/2]⁺=586.2.

### Synthesis of Conjugate 184

Int-317 (16.9 mg, 0.01448 mmol in DMF (0.5 mL) was added to SEQ ID NO: 80 (100 mg, 7.75 mL in PBS at pH 8.4) as described in the general conjugation procedure. Maldi TOF analysis of the purified final product gave an average mass of 60,365 Da. (DAR=5). Yield 49 mg, 49%.

### Synthesis of Int-259

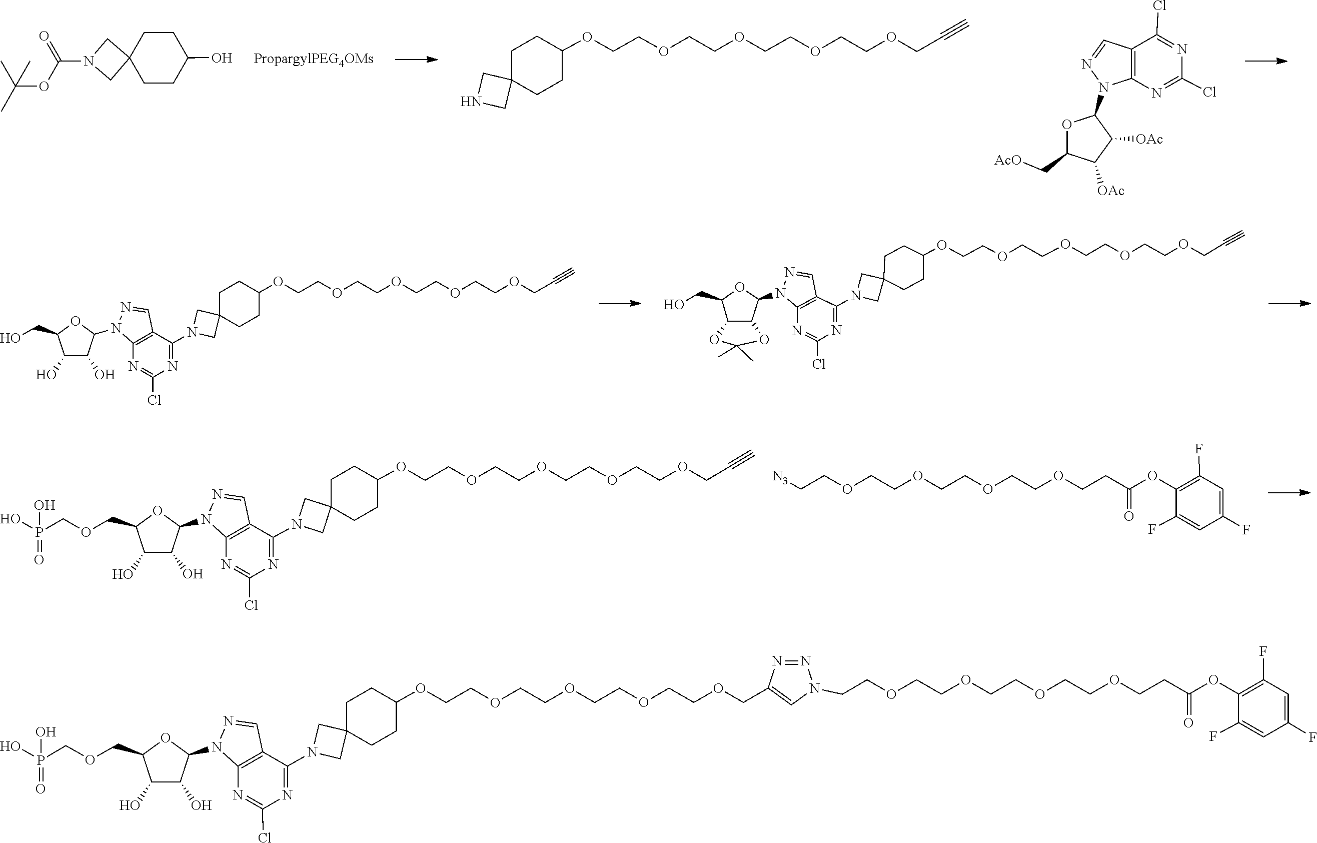

### Step a

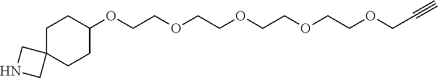 [SMILES: C#CCOCCOCCOCCOCCOC1CCC2(CC1)CNC2]

To a solution of tert-butyl 7-hydroxy-2-azaspiro[3.5]nonane-2-carboxylate (500 mg, 2.07 mmol) in DMF (20 mL) at 0° C. was added NaH (124 mg, 1.5 mmol, 60% in mineral oil,) in portions, and the mixture was stirred for 1 h at room temperature. then PropargylPEG4-mesylate (643 mg, 2.07 mmol) was added to it and stirred for 16 h at room temperature. The resulting solution was quenched with saturated ammonium chloride solution (20 mL) and extracted with EtOAc (3×20 mL). Combined organic extracts were washed with water, brine, dried over sodium sulfate, and concentrated to give the crude product which was further purified by semi-preparative HPLC (ACN: H200 to 100%). The product was a light-yellow viscous oil. Yield 763 mg, 83%. Ion(s) found by LCMS: [M−^{t}Bu+H]⁺=400.2. Intermediate N-Boc derivative (763 mg, 1.68 mmol) was dissolved in DCM (10 mL) and TFA (3 mL). The resulting mixture was stirred for 4 h at room temperature then solvent was removed under reduced pressure and dried under vacuum to yield the compound as its TFA salt. The product was a yellow oil. Yield 683 mg, 100%. Ion(s) found by LCMS: [M+H]⁺=356.2.

### Step b

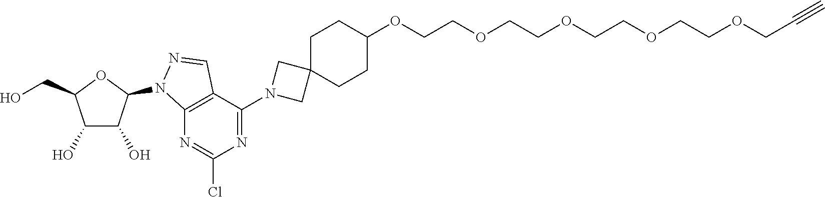 [SMILES: C#CCOCCOCCOCCOCCOC1CCC2(CC1)CN(c1nc(Cl)nc3c1cnn3[C@@H]1O[C@H](CO)[C@@H](O)[C@H]1O)C2]

A mixture of intermediate B (447 mg, 1 mmol), amine from previous step (469 mg, 1.2 mmol), and DIEA (0.28 mL, 2 mmol) in MeOH was heated at 50° C. for 1 h. After complete consumption of the starting materials, the reaction mixture was cooled to room temperature and volatiles were removed by rotatoreoration. The crude residue was dissolved in EtOAc (50 mL) and washed with water and brine. The organic layer was dried over sodium sulfate and concentrated to yield the crude product as a yellow viscous oil, which was used in the next step without further purification. Ion(s) found by LCMS: [M+H]⁺=766.2. The triacetate intermediate was dissolved in MeOH (6 mL), treated with potassium carbonate (0.48 g, 3.50 mmol), stirred at room temperature for 2 h, filtered through a short pad of celite, washed with methanol, and concentrated. The crude material obtained was purified by semi-preparative HPLC (5% to 100% ACN/water, 0.1% TFA modifier) to yield the product as an off-white foam. Yield 548 mg, 85.61%. Ion(s) found by LCMS: [M+H]⁺=640.2.

### Step c

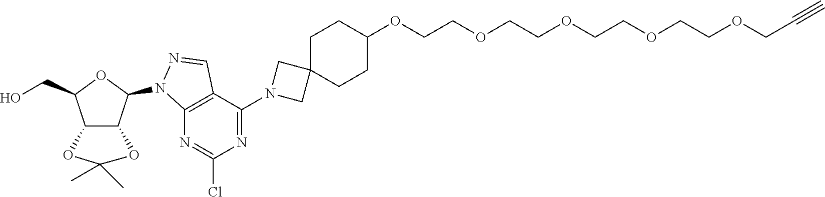 [SMILES: C#CCOCCOCCOCCOCCOC1CCC2(CC1)CN(c1nc(Cl)nc3c1cnn3[C@@H]1O[C@H](CO)[C@H]3OC(C)(C)O[C@H]31)C2]

To a solution of the triol from previous step (540 mg, 0.84 mmol) and 2,2-dimethoxypropane (440 mg, 4.2 mmol) in acetone (20 mL) at room temperature was added toluene sulfonic acid (145 mg, 0.84 mmol). The reaction was stirred for two hours then concentrated under reduced pressure. The crude material was dissolved in EtOAc (20 mL) and washed with saturated NaHCO₃. The organic layer was separated and dried over sodium sulfate, filtered, and concentrated in vacuo to provide crude acetonide derivative, which was purified by flash chromatography (DCM: 10% MeOH in DCM) to yield product. The product was a white foam. Yield 420 mg, 73%. Ion(s) found by LCMS: [M+H]⁺=680.2.

### Step d

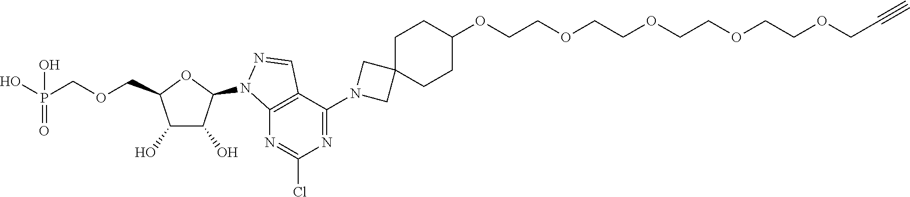 [SMILES: C#CCOCCOCCOCCOCCOC1CCC2(CC1)CN(c1nc(Cl)nc3c1cnn3[C@@H]1O[C@H](COCP(=O)(O)O)[C@@H](O)[C@H]1O)C2]

A solution of acetonide derivative from previous step (250 mg, 0.367 mmol) and [bis(tert-butoxy)carbonyl]methyl (trifluoromethyl)sulfonate (262 mg, 0.735 mmol) in dry DMF (3 mL) was cooled using ice-water bath. 60% NaH in mineral oil (88 mg, 2.2 mmol) was added to this and gradually warmed to room temperature and stirred for 1 h. The reaction mixture was quenched with saturated ammonium chloride then extracted with EtOAc. The organic phase was washed with brine, dried over sodium sulfate, and concentrated to give crude product which was used in the next step without purification. Ion(s) found by LCMS: [M+H]⁺=886.2. Crude product was dissolved in 50% aqueous TFA (10 mL) and stirred at room temperature for 12 h, concentrated, and purified by semi-preparative HPLC (10-100% ACN: H₂O, 0.1% TFA modifier). The product was a white solid. Yield 146 mg, 54.1%. Ion(s) found by LCMS: [M+H]⁺=734.2.

### Step e

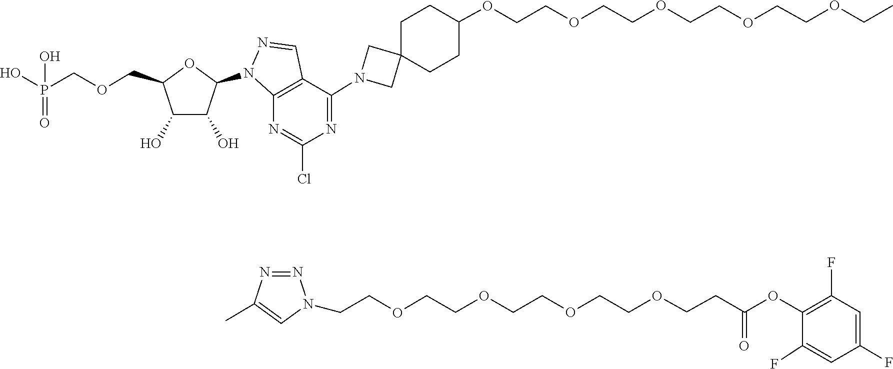

To a solution of product from the previous step (40 mg, 0.054 mmol) and intermediate A (23 mg, 0.054 mmol). dissolved in DMF:H₂O (1:3, 1.5 mL) were cooled to 0° C. To this a pre-mixed solution of a solution of copper(II) sulfate (1 mg, 0.005 mmol), sodium ascorbate (32 mg, 0.163 mmol), and BTTA (5 mg, 0.011 mmol) dissolved in water (0.5 mL) was added and stirred for 5 minutes at the same temperature and gradually warmed to room temperature and stirred room temperature for 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 39 mg, 61.95%. Ion(s) found by LCMS [(M+2H)/2]⁺=578.2.

### Synthesis of Conjugate 133a

Trifluorophenol ester (15 mg, 0.014 mmol, Int-259) in DMF (0.5 mL) was added to Fc carrier SEQ ID NO: 13 (100 mg, 3.44 mL in PBS at pH 7.4) as described in the conjugation procedure for Conjugate 71. %. Maldi TOF analysis of the purified final product gave an average mass of 63689 Da. (DAR=5.5).

Yield 34.7 mg, 34.7%.

Synthesis of Conjugate 133b Trifluorophenol ester (34 mg, 0.028 mmol, Int-259) in DMF (0.5 mL) was added to Fc carrier SEQ ID NO: 80 (200 mg, in 13.3 mL in PBS at pH 7.4) as described in the conjugation procedure for Conjugate 71. Maldi TOF analysis of the purified final product gave an average mass of 59411 Da. (DAR=4.1).

Yield 105 mg, 52.5%.

Synthesis of Conjugate 133c Int-259 (12.6 mg, 0.011 mmol) in DMF (0.5 mL) was added to Palivizumab full length antibody SEQ ID NOs: 113 and 114) (200 mg, 11.9 mL in PBS at pH 8.4) as described in the conjugation procedure for Conjugate 71. Maldi TOF analysis of the purified final product gave an average mass of 150,599 Da. (DAR=3.3). Yield 102 mg, 51%.

### Synthesis of Int-281

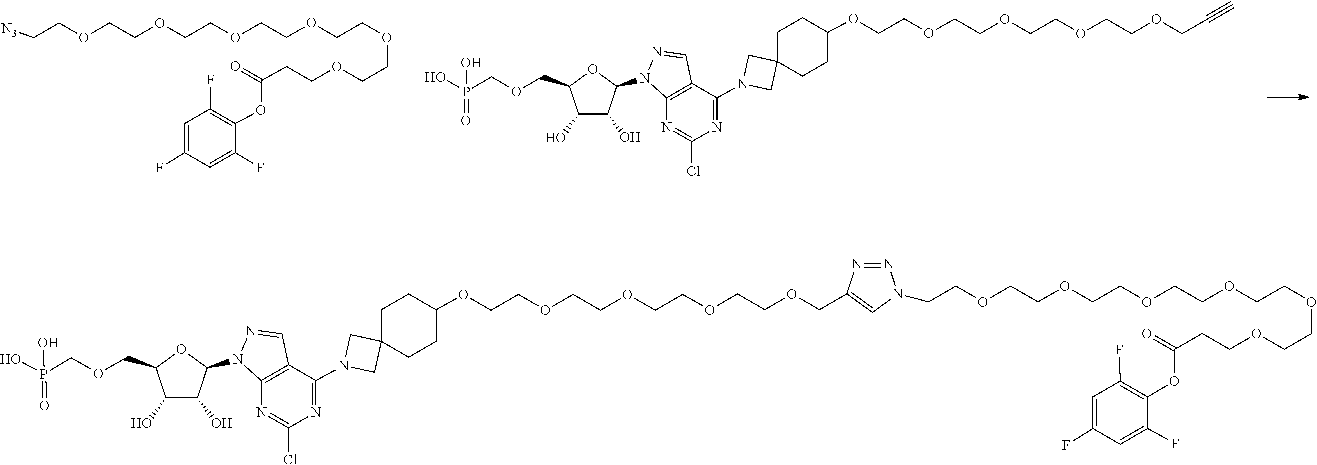 [SMILES: [N-]=[N+]=NCCOCCOCCOCCOCCOCCOCCC(=O)Oc1c(F)cc(F)cc1F ; C#CCOCCOCCOCCOCCOC1CCC2(CC1)CN(c1nc(Cl)nc3c1cnn3[C@@H]1O[C@H](COCP(=O)(O)O)[C@@H](O)[C@H]1O)C2 ; O=C(CCOCCOCCOCCOCCOCCOCCn1cc(COCCOCCOCCOCCOC2CCC3(CC2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

EDC (490 mg, 3.16 mmol) was added, in 4 portions, to a stirring mixture of azido-peg6-carboxylic acid (1 g, 2.65 mmol) and 2,4,6-trifluorophenol (468 mg, 3.2 mmol) in DCM (17 mL). The mixture was stirred for 2 hours at ambient temperature. The organic phase was washed with DI water (30 mL) and the aqueous phase was back extracted with DCM (2×20 mL). The combined organic extracts were dried over sodium sulfate, filtered, and concentrated. The crude product mixture was purified by silica gel chromatography (0-80% EtOAc in Hexanes) to afford the product as a clear oil. 842 mg, 63%. Yield Ion(s) found by LCMS: [M+Na]⁺=532.2.

### Step b

The title compound was prepared analogously Int-259 where azidoPEG₄TFP ester was replaced with azidoPEG₆TFP ester prepared as described in step a. The product was a white solid. Ion(s) found by LCMS: [(M+2H)/2]⁺=622.2.

### Synthesis of Conjugate 149

Int-281 (7 mg, 0.0058 mmol) was added to SEQ ID NO: 80 (40 mg, 4 mL in PBS at pH 8.4) as described in the general conjugation procedure. Maldi TOF analysis of the purified final product gave an average mass of 59,372 Da. (DAR=3.8). Yield 18 mg, 45%.

### Synthesis of Int-282

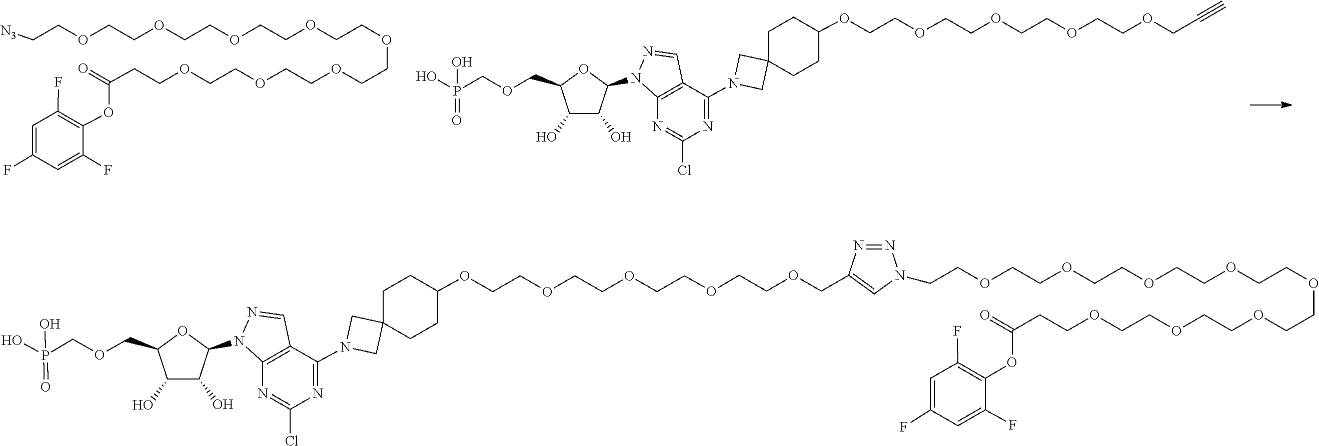 [SMILES: [N-]=[N+]=NCCOCCOCCOCCOCCOCCOCCOCCOCCC(=O)Oc1c(F)cc(F)cc1F ; C#CCOCCOCCOCCOCCOC1CCC2(CC1)CN(c1nc(Cl)nc3c1cnn3[C@@H]1O[C@H](COCP(=O)(O)O)[C@@H](O)[C@H]1O)C2 ; O=C(CCOCCOCCOCCOCCOCCOCCOCCOCCn1cc(COCCOCCOCCOCCOC2CCC3(CC2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

### Step a

EDC (397 mg, 2.56 mmol) was added, in 4 portions, to a stirring mixture of azido-peg8-carboxylic acid (1 g, 2.14 mmol) and 2,4,6-trifluorophenol (380 mg, 2.56 mmol) in DCM (15 mL). The mixture was stirred for 2 hours at ambient temperature. The organic phase was washed with DI water (30 mL) and the aqueous phase was back extracted with DCM (2×20 mL). The combined organic extracts were dried over sodium sulfate, filtered, and concentrated. The crude product mixture was purified by silica gel chromatography (0-80% EtOAc in Hexanes) to afford the product as a clear oil. 677 mg, 53%. Yield. Ion(s) found by LCMS: [M+H}⁺=598.2.

### Step b

The title compound was prepared analogously Int-259 where intermediate A was replaced with azidoPEG₈TFP ester prepared as described in step a. The product was a white solid. Ion(s) found by LCMS: [(M+2H)/2]⁺=666.3.

### Synthesis of Conjugate 150

Int-282 (9.6 mg, 0.0072 mmol) was added to SEQ ID NO: 80 (40 mg, 4 mL in PBS at pH 8.4) as described in the general conjugation procedure. Maldi TOF analysis of the purified final product gave an average mass of 59,958 Da. (DAR=4). Yield 23 mg, 57%.

### Synthesis of Int-283

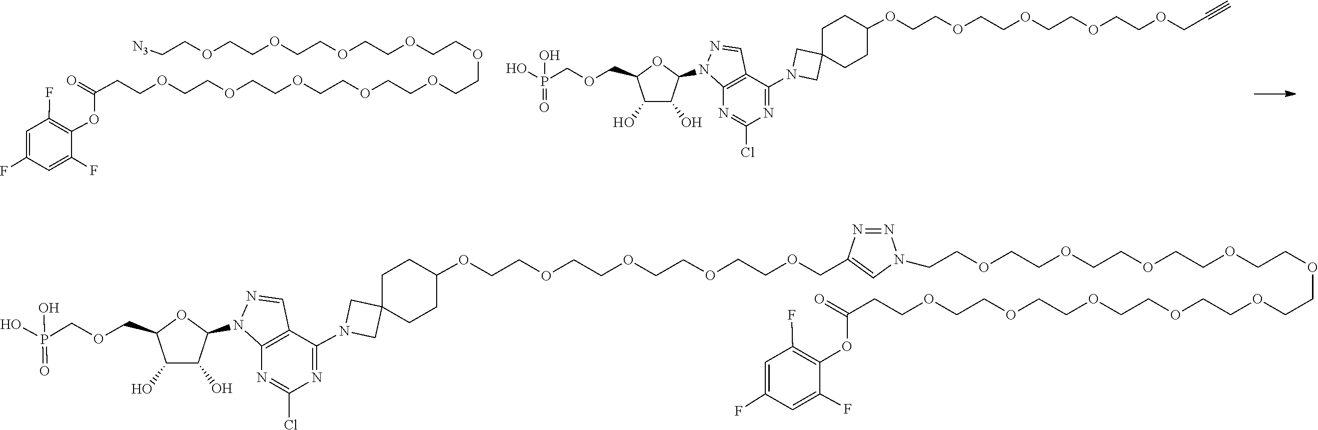 [SMILES: [N-]=[N+]=NCCOCCOCCOCCOCCOCCOCCOCCOCCOCCOCCC(=O)Oc1c(F)cc(F)cc1F ; C#CCOCCOCCOCCOCCOC1CCC2(CC1)CN(c1nc(Cl)nc3c1cnn3[C@@H]1O[C@H](COCP(=O)(O)O)[C@@H](O)[C@H]1O)C2 ; O=C(CCOCCOCCOCCOCCOCCOCCOCCOCCOCCOCCn1cc(COCCOCCOCCOCCOC2CCC3(CC2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

### Step a

EDC (340 mg, 3.16 mmol) was added, in 4 portions, to a stirring mixture of azido-peg10-carboxylic acid (1 g, 1.79 mmol) and 2,4,6-trifluorophenol (319 mg, 1.79 mmol) in DCM (15 mL). The mixture was stirred for 2 hours at ambient temperature. The organic phase was washed with DI water (30 mL) and the aqueous phase was back extracted with DCM (2×20 mL). The combined organic extracts were dried over sodium sulfate, filtered, and concentrated. The crude product mixture was purified by silica gel chromatography (0-80% EtOAc in Hexanes) to afford the product as a clear oil. 536 mg, 43% yield. Ion(s) found by LCMS: [M+Na]⁺=708.2.

### Step b

The title compound was prepared analogously Int-259 where intermediate A was replaced with azidoPEG₁₀TFP ester prepared as described in step a. The product was a white solid. Ion(s) found by LCMS: [(M+2H)/2]⁺=710.4.

### Synthesis of Conjugate 151

Int-283 (12.3 mg, 0.0087 mmol) was added to SEQ ID NO: 80 (60 mg, 6 mL in PBS at pH 8.4) as described in the general conjugation procedure. Maldi TOF analysis of the purified final product gave an average mass of 60,165 Da. (DAR=3.9). Yield 23 mg, 57%.

### Synthesis of Int-284

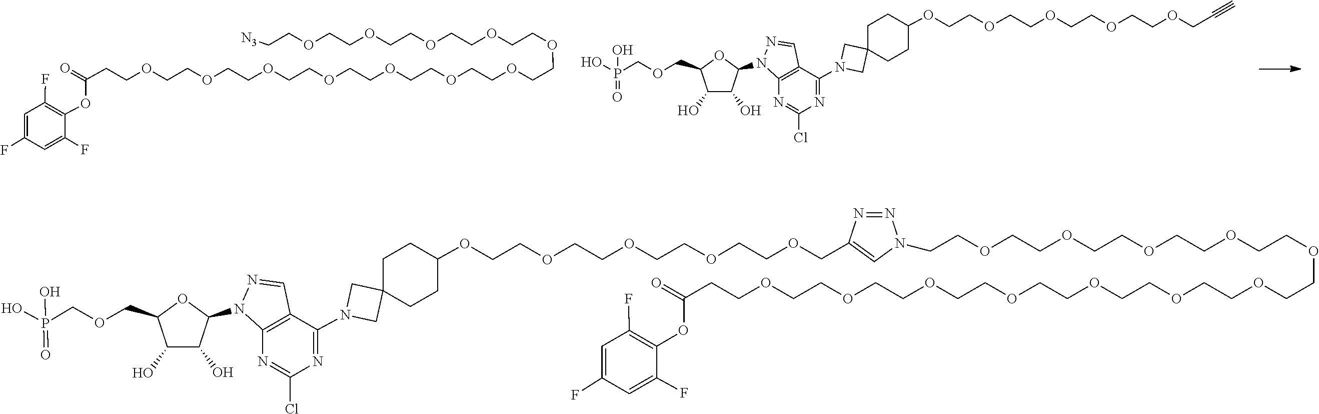 [SMILES: [N-]=[N+]=NCCOCCOCCOCCOCCOCCOCCOCCOCCOCCOCCOCCOCCC(=O)Oc1c(F)cc(F)cc1F ; C#CCOCCOCCOCCOCCOC1CCC2(CC1)CN(c1nc(Cl)nc3c1cnn3[C@@H]1O[C@H](COCP(=O)(O)O)[C@@H](O)[C@H]1O)C2 ; O=C(CCOCCOCCOCCOCCOCCOCCOCCOCCOCCOCCOCCOCCn1cc(COCCOCCOCCOCCOC2CCC3(CC2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

### Step a

EDC (0.29 g, 1.86 mmol) was added, in 4 portions, to a stirring mixture of azido-peg4-carboxylic acid (2 g, 6.9 mmol) and 2,4,6-trifluorophenol (1.2 g, 8.2 mmol) in DCM (10 mL). The mixture was stirred for 2 hours at ambient temperature. The organic phase was washed with DI water (30 mL) and the aqueous phase was back extracted with DCM (2×, 20 mL). The combined organic extracts were dried over sodium sulfate, filtered and concentrated. The crude product mixture was purified by RPLC (10% to 100% ACN/water, 0.1% TFA modifier) to afford the product as a clear oil. 805 mg, 67%. Ion(s) found by LCMS: [M+H]⁺=774.2.

### Step b

The title compound was prepared analogously to Int-259 where intermediate A was replaced with azidoPEG₁₂TFP ester prepared as described in step a. The product was a white solid. Ion(s) found by LCMS: [(M+2H)/2]⁺=754.4.

Synthesis of Conjugate 152 Int-284 (13 mg, 0.0087 mmol) was added to SEQ ID NO: 80 (60 mg, 6 mL in PBS at pH 8.4) as described in the general conjugation procedure. Maldi TOF analysis of the purified final product gave an average mass of 61, 586 Da. (DAR=4.7). Yield 30 mg, 49%.

### Synthesis of Int-285

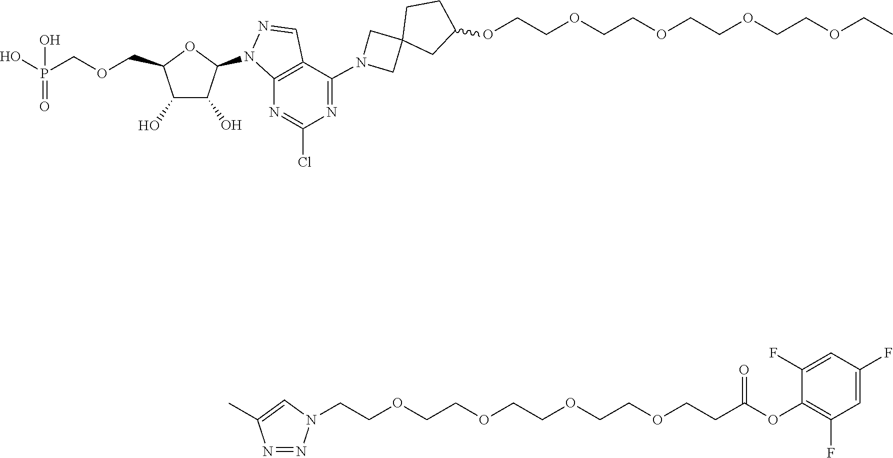

The title compound was prepared analogously to Int-259, where the cyclohexyl spirocyclic azetidine is replaced with racemic cyclopentyl spirocyclic azetidine in step a of that procedure.

### Synthesis of Int-288

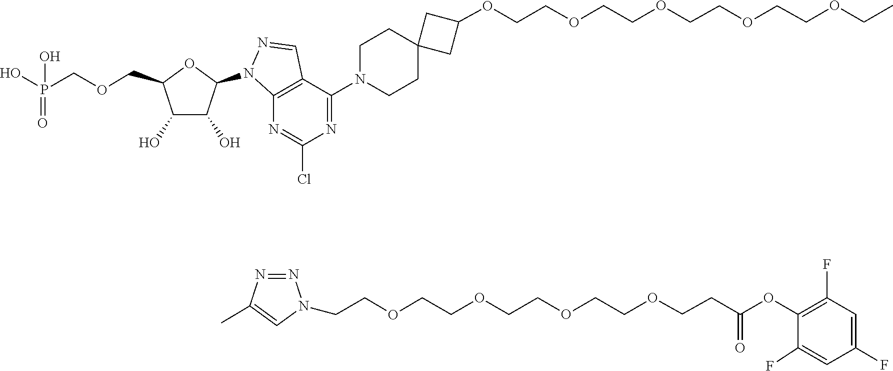

The title compound was prepared analogously Int-259 where tert-butyl 7-hydroxy-2-azaspiro[3.5]nonane-2-carboxylate was replaced with tert-butyl 2-hydroxy-7-azaspiro[3.5]nonane-7-carboxylate. The product was a white solid. Ion(s) found by LCMS: [(M+2H)/2]⁺=578.4.

### Synthesis of Conjugate 155

Int-288 (13.38 mg, 0.01158 mmol) described in was added to Fc carrier SEQ ID NO: 80 (80 mg, 8.33 mL in PBS at pH 8.4) as described in the general conjugation procedure. Maldi TOF analysis of the purified final product gave an average mass of 61,043 Da. (DAR=5.8). Yield 39 mg, 48%.

### Synthesis of Int-289

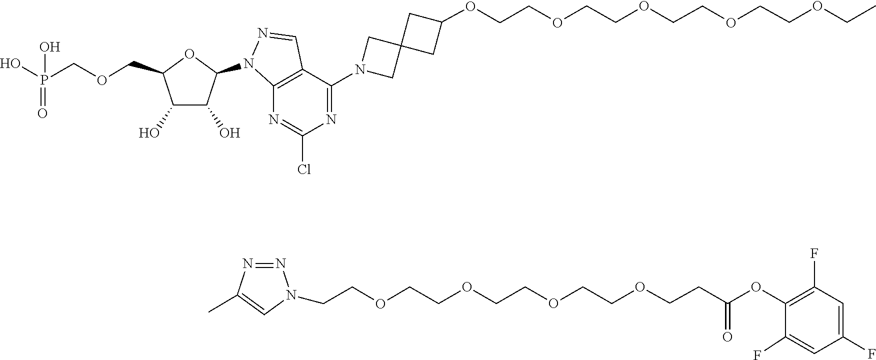

The title compound was prepared analogously Int-259 where tert-butyl 7-hydroxy-2-azaspiro[3.5]nonane-2-carboxylate was replaced with tert-butyl 6-hydroxy-2-azaspiro[3.3]heptane-2-carboxylate. The product was a white solid. Ion(s) found by LCMS: [(M+2H)/2]⁺=564.2.

### Synthesis of Conjugate 156

Int-289 (13.06 mg, 0.01158 mmol) was added to SEQ ID NO: 80 (80 mg, 8.33 mL in PBS at pH 8.4) as described in the general conjugation procedure. Maldi TOF analysis of the purified final product gave an average mass of 58,919 Da. (DAR=3.8). Yield 38 mg, 47%.

### Synthesis of Int-303

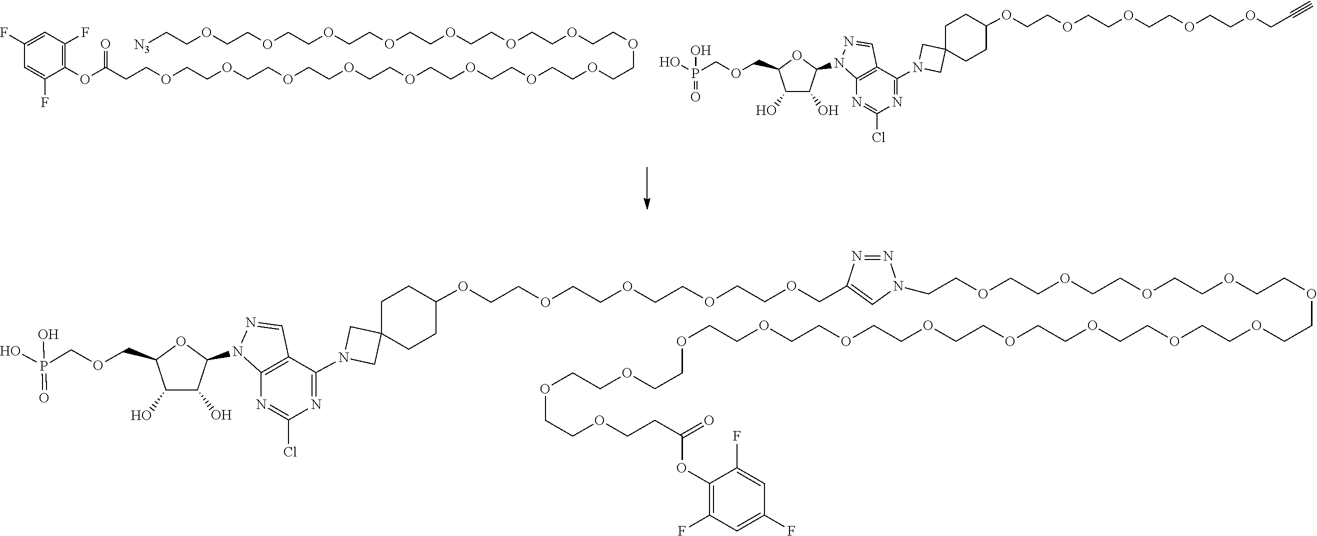

### Step a

EDC (114 mg, 0.73 mmol) was added, in 4 portions, to a stirring mixture of azido-peg16-carboxylic acid (500 mg, 0.61 mmol), and 2,4,6-trifluorophenol (108 mg, 0.74 mmol) in DCM (20 mL). The mixture was stirred for 2 hours at ambient temperature. The organic phase was washed with DI water (30 mL) and the aqueous phase was back extracted with DCM (2×20 mL). The combined organic extracts were dried over sodium sulfate, filtered and concentrated. The crude product mixture was purified by RPLC (10% to 100% ACN/water, 0.1% TFA modifier) to afford the product as a clear oil. 400 mg, 59%. Ion(s) found by LCMS: [M+H]⁺=950.2.

### Step b

The title compound was prepared analogously to Int-259, where azidoPEG₄TFP ester was replaced with azidoPEG₁₆TFP ester prepared as described in step a. The product was a white solid. Ion(s) found by LCMS: [(M+2H)/2]⁺=842.4.

### Synthesis of Conjugate 170

Int-303 (9.4 mg, 0.00556 mmol) was added to SEQ ID NO: 80 (50 mg, 3.8 mL in PBS at pH 8.4) as described in the general conjugation procedure. Maldi TOF analysis of the purified final product gave an average mass of 64,391 Da. (DAR=6). Yield 28 mg, 57%.

### Synthesis of Int-304

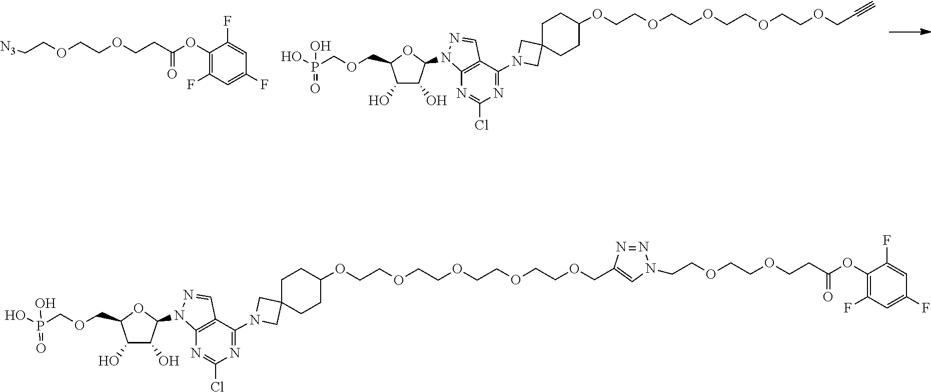 [SMILES: [N-]=[N+]=NCCOCCOCCC(=O)Oc1c(F)cc(F)cc1F ; C#CCOCCOCCOCCOCCOC1CCC2(CC1)CN(c1nc(Cl)nc3c1cnn3[C@@H]1O[C@H](COCP(=O)(O)O)[C@@H](O)[C@H]1O)C2 ; O=C(CCOCCOCCn1cc(COCCOCCOCCOCCOC2CCC3(CC2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

### Step a

EDC (340 mg, 3.16 mmol) was added, in 4 portions, to a stirring mixture of azido-peg2-carboxylic acid (363 mg, 1.79 mmol) and 2,4,6-trifluorophenol (319 mg, 1.79 mmol) in DCM (15 mL). The mixture was stirred for 2 hours at ambient temperature. The organic phase was washed with DI water (30 mL) and the aqueous phase was back extracted with DCM (2×20 mL). The combined organic extracts were dried over sodium sulfate, filtered, and concentrated. The crude product mixture was purified by silica gel chromatography (0-80% EtOAc in Hexanes) to afford the product as a clear oil. 411 mg, 69%. Yield Ion(s) found by LCMS: [M+H]⁺=334.2.

### Step b

The title compound was prepared analogously Int-259, where intermediate A was replaced with azidoPEG₂TFP ester prepared as described in step a. The product was a white solid. Ion(s) found by LCMS: [M+H]⁺=1067.2

### Synthesis of Conjugate 171

Int-304 (7.8 mg, 0.005724 mmol) described in was added to SEQ ID NO: 80 (50 mg, 3.8 mL in PBS at pH 8.4) as described in the general conjugation procedure. Maldi TOF analysis of the purified final product gave an average mass of 61,397 Da. (DAR=6.7). Yield 27 mg, 53%.

### Synthesis of Conjugate 153

A solution of Int-285 (9.1 mg, 0.0072 mmol) dissolved in DMF (1 mL), was added to a solution of SEQ ID NO: 80 (50 mg, 0.00091 mmol, in PBS at pH 7.4, 17.3 mg/mL) at room temperature. The pH of the resulting solution was adjusted to ˜8.5 with borate buffer (300 uL, 1M, pH 8.5). The homogeneous colorless reaction was rocked gently for 3 h, then purified by dialysis (two times in 200 mM arginine, 120 mM NaCl, 1% Sucrose, pH 6.0). Maldi TOF analysis of the purified final product gave an average mass of 59,997 Da (DAR=4.8). Yield: 22 mg, 44%.

### Synthesis of Int-268

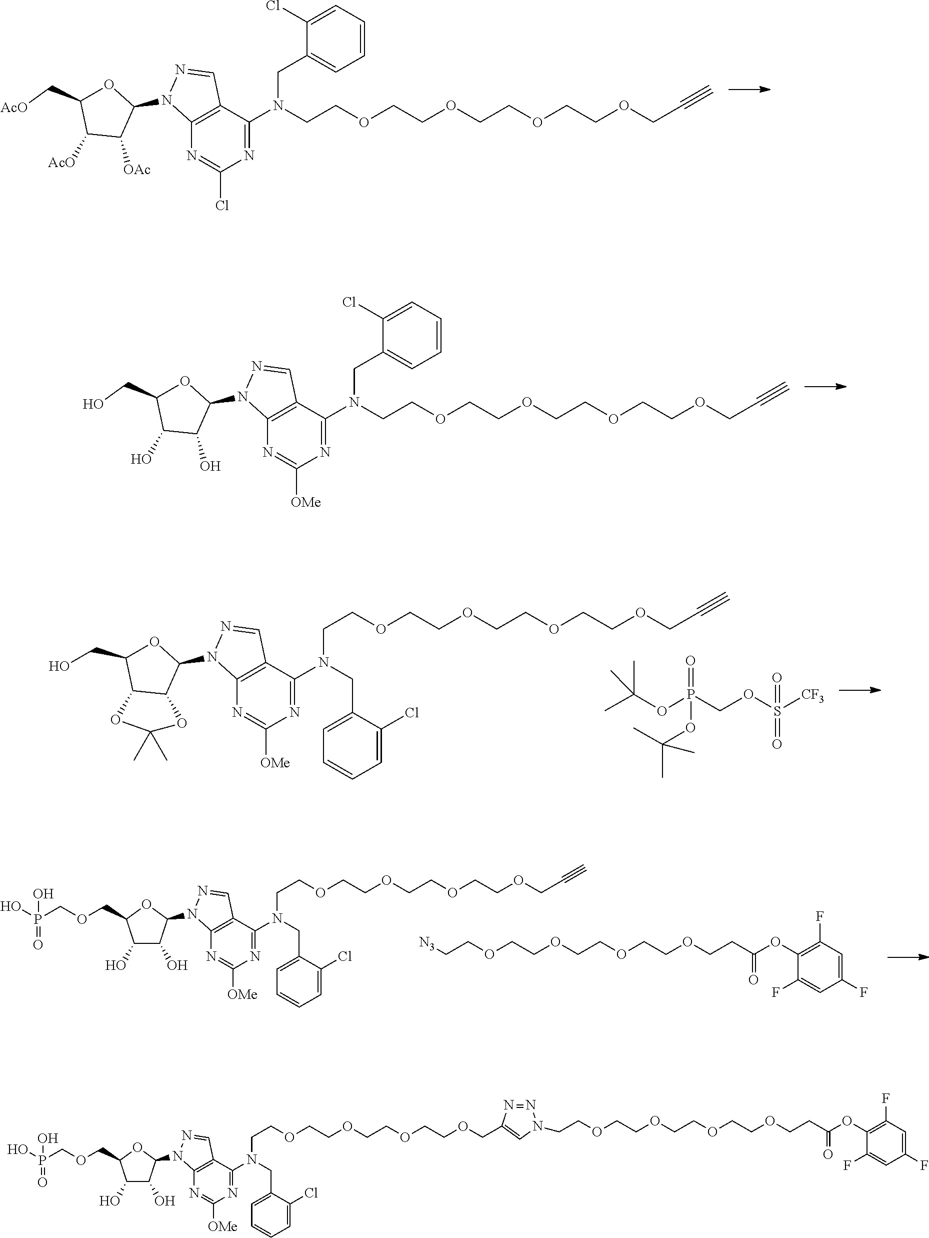

### Step a

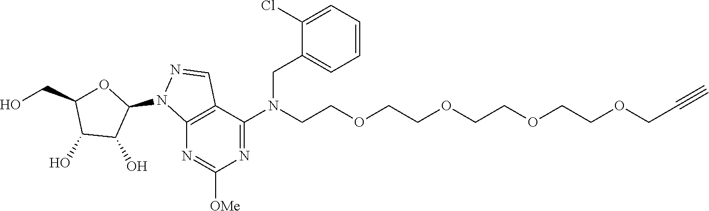 [SMILES: C#CCOCCOCCOCCOCCN(Cc1ccccc1Cl)c1nc(OC)nc2c1cnn2[C@@H]1O[C@H](CO)[C@@H](O)[C@H]1O]

Triacetate derivative described in step b of Int-255 (600 mg, 0.78 mmol) was dissolved in MeOH (10 mL) and NaOMe in MeOH (25% in MeOH, 2.2 mL) was added to it. The resulting mixture was stirred at room temperature 18 h. The mixture was concentrated, and the residue was partitioned between EtOAc (50 mL) and water. The organic layer was washed with brine, dried over anhydrous sodium sulfate and concentrated. The residue was purified by normal phase column chromatography using DCM to 10% MeOH in DCM, off-white foam. 456 mg, 91.9%. Ion(s) found by LCMS: [M+H]⁺=636.3.

### Step b

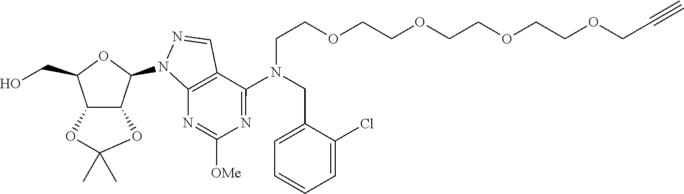 [SMILES: C#CCOCCOCCOCCOCCN(Cc1ccccc1Cl)c1nc(OC)nc2c1cnn2[C@@H]1O[C@H](CO)[C@H]2OC(C)(C)O[C@H]21]

To a solution of the triol (456 mg, 0.72 mmol) and 2,2-dimethoxypropane (224 mg, 4.2 mmol) in acetone (20 mL) at room temperature was added toluene sulfonic acid (123 mg, 0.72 mmol). The reaction was stirred for two hours then concentrated under reduced pressure. The crude material was dissolved in EtOAc (20 mL) and washed with saturated NaHCO₃. The organic layer was separated and dried over sodium sulfate, filtered and concentrated in vacuo to provide crude acetonide derivative, which was purified by flash chromatography (DCM: 10% MeOH in DCM) to yield compound. The product was a white foam. Yield 426 mg, 87.9%. Ion(s) found by LCMS: [M+H]⁺=676.2.

### Step c

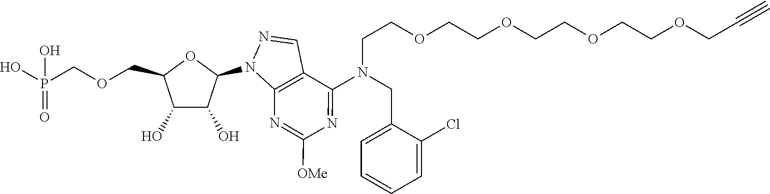 [SMILES: C#CCOCCOCCOCCOCCN(Cc1ccccc1Cl)c1nc(OC)nc2c1cnn2[C@@H]1O[C@H](COCP(=O)(O)O)[C@@H](O)[C@H]1O]

A solution of acetonide derivative from previous step (300 mg, 0.443 mmol) and [bis(tert-butoxy)carbonyl]methyl (trifluoromethyl)sulfonate (474 mg, 1.33 mmol) in dry DMF (5 mL) was cooled using ice-water bath. 60% NaH in mineral oil (107 mg, 2.66 mmol) was added to this and stirred at room temperature for 2 h. The reaction mixture was quenched with saturated ammonium chloride and extracted with EtOAc. The organic phase was washed with brine and dried over sodium sulfate concentrated to give crude product which was used in the next step without purification. Observed LCMS: 882.2 Crude product was dissolved in 50% aqueous TFA (10 mL) and stirred at room temperature for 12 h, concentrated and the residue was purified by semi-preparative HPLC (10-100% ACN: H₂O, 0.1% TFA modifier). The product was a white solid. Yield 279 mg, 86.13%. Ion(s) found by LCMS: [M+H]+=730.2.

### Step d

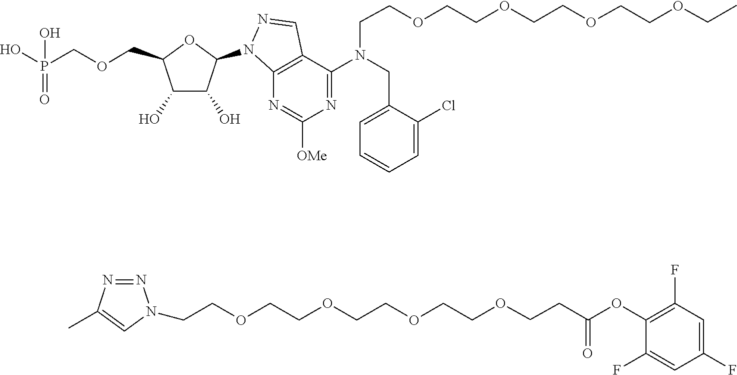

A solution of the product from the previous step ((40 mg, 0.055 mmol) and intermediate A (23 mg, 0.055 mmol) were dissolved in DMF:H₂O (1:3, 1.5 mL) and cooled to 0° C. A pre-mixed solution of a solution of copper(II) sulfate (1 mg, 0.005 mmol), sodium ascorbate (32 mg, 0.163 mmol), and BTTA (5 mg, 0.011 mmol) dissolved in water (0.5 mL) was added, and stirred for 5 minutes at the same temperature then gradually warmed to room temperature and stirred for 15 minutes. When the reaction was complete (by HPLC), the mixture was quenched with a few drops of glacial acetic acid and 125 mM EDTA (pH 6). The product was purified by reversed phase HPLC (10% to 100% ACN/water, 0.1% TFA modifier). The product was a white solid. Yield 57 mg, 90.3%. Ion(s) found by LCMS [(M+2H)/2]⁺=576.2.

### Synthesis of Conjugate 138

Trifluorophenol ester (17 mg, 0.0137 mmol, Int-268) in DMF (0.5 mL) was added to Fc carrier SEQ ID NO: 13 (100 mg, 2.78 mL, 0.00172 mmol) in PBS at pH 7.4 as described in the conjugation procedure for Conjugate 71. Maldi TOF analysis of the purified final product gave an average mass of 65,321 Da. (DAR=7.1). Yield 49.5 mg, 49.5%.

### Synthesis of Int-273

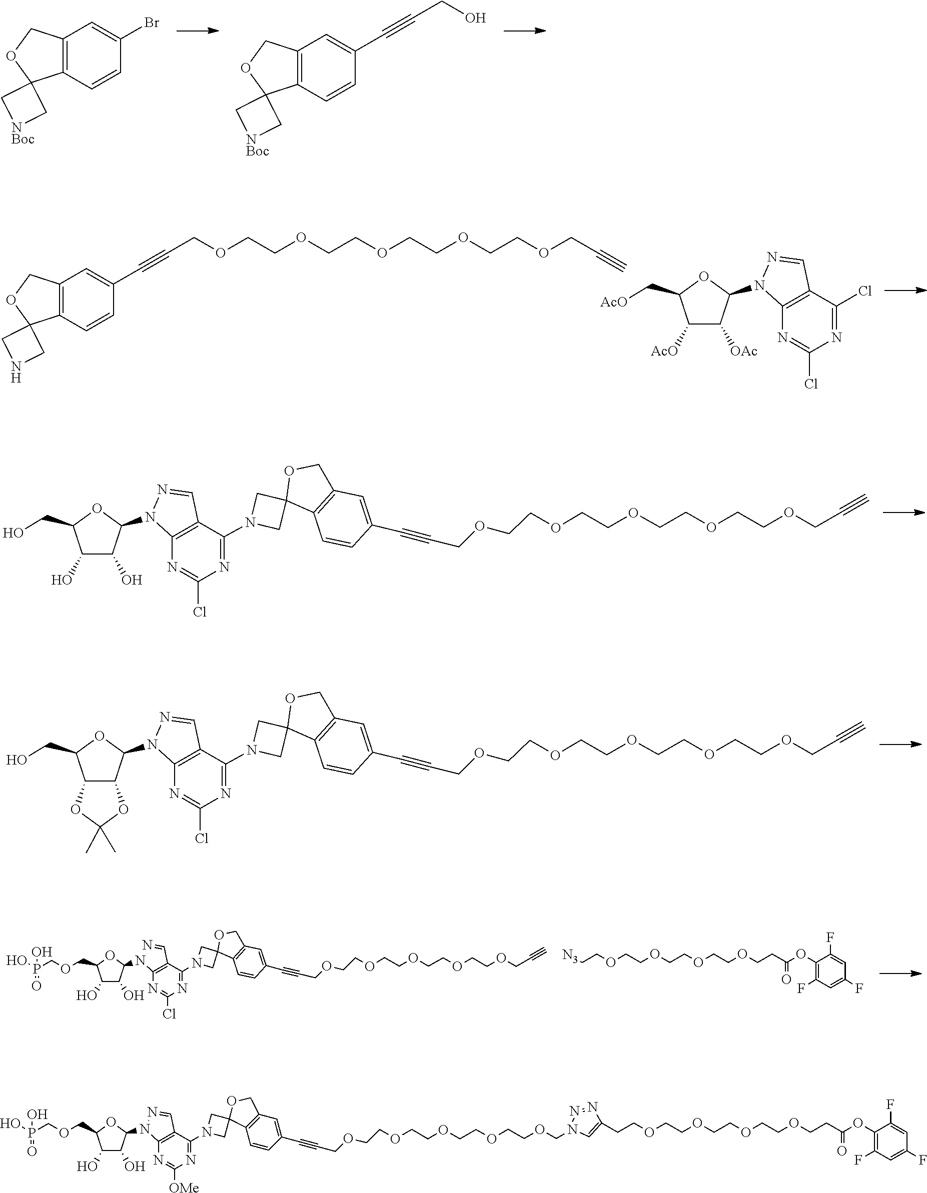

### Step a

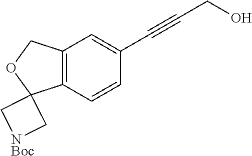 [SMILES: CC(C)(C)OC(=O)N1CC2(C1)OCc1cc(C#CCO)ccc12]

Pd(PPh₃)₂Cl₂ (0.025 g, 0.04 mmol) and CuI (0.027 g, 0.14 mmol) were placed in a round bottom flask and purged with nitrogen for 5 minutes. Triethylamine (20 mL) and bromo compound (2.5 g, 7.3 mmol) were added to the mixture, under nitrogen atmosphere, then stirred for 5 minutes at room temperature. Propargyl alcohol (0.500 g, 8.8 mmol) was added to the mixture then stirred the for 6 hours at 80° C. The reaction mixture was cooled to room temperature, quenched with NaHCO₃ (15 mL, sat. aqueous.), extracted with EtOAc (3×20 mL), washed with brine, dried over sodium sulfate, filtered, and purified by silica gel column chromatography using DCM to 10% MeOH in DCM. The product was a yellow solid. Yield 560 mg, 50.3%. Ion(s) found by LCMS: [M−^{t}Bu+H]⁺=260.2.

### Step b

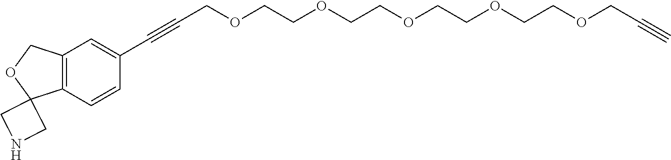 [SMILES: C#CCOCCOCCOCCOCCOCC#Cc1ccc2c(c1)COC21CNC1]

To a solution of the alcohol derivative from previous step (360 mg, 1.146 mmol) in DMF (10 mL), under nitrogen, at 0° C., was added NaH (91 mg, 2.28 mmol, 60% in mineral oil). The resulting mixture was stirred at the same temperature for 10 minutes. To this was added a solution of propargyl PEG4 mesylate (425 mg, 1.37 mmol) in DMF (1 mL), dropwise, then gradually warmed to room temperature, and stirred overnight. The reaction mixture was quenched with ammonium chloride and extracted using EtOAc (3×). The combined organic extracts were washed with brine, and dried over sodium sulfate. Removal of the solvent followed by silica gel column chromatography (DCM to 10% MeOH in DCM gave the desired product, which was a yellow foam. Yield 430 mg, 71.3%. Ion(s) found by LCMS: [M-Boc+H]⁺=430.2. 430 mg (0.81 mmol) of the intermediate Boc derivative was dissolved in DCM (20 mL) and TFA (5 mL). The resulting mixture was stirred at room temperature for 1 hr. The material obtained was dried and used in the next step without purification. The product was a yellow oil. Yield 360 mg (quantitative yield). Ion(s) found by LCMS: [M+H]⁺=430.2.

### Step c

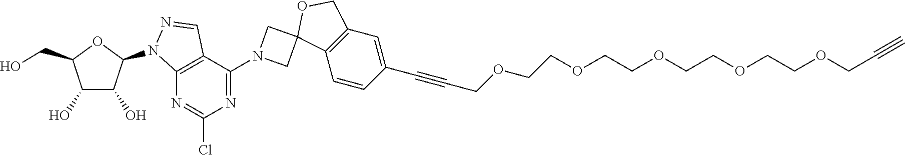 [SMILES: C#CCOCCOCCOCCOCCOCC#Cc1ccc2c(c1)COC21CN(c2nc(Cl)nc3c2cnn3[C@@H]2O[C@H](CO)[C@@H](O)[C@H]2O)C1]

A mixture of intermediate B (300 mg, 0.671 mmol), the amine from previous step (345 mg, 0.805 mmol), and DIEA (0.35 mL) in MeOH (10 mL) was heated at 50° C. for 1 h. After complete consumption of the starting materials, the reaction mixture was cooled to room temperature and volatiles were removed by rotatory evaporation. The crude residue was dissolved in EtOAc (50 mL) and washed with water and brine. The organic layer was dried over sodium sulfate and concentrated under reduced pressure to yield the desired product as a yellow foam which was used in the next step without further purification. Ion(s) found by LCMS: [M+H]⁺=840.2. The triacetate intermediate was dissolved in MeOH (6 mL), potassium carbonate (0.330 g, 2.35 mmol) was added, and the reaction stirred at room temperature for 2 h. The crude mixture was filtered through a short pad of celite and washed with methanol and concentrated. The crude material was then purified by semi-preparative HPLC (5% to 100% ACN/water, 0.1% TFA modifier) giving the desired compound as an off-white foam. Yield 430 mg, 80.7%. Ion(s) found by LCMS: [M+H]⁺=714.2.

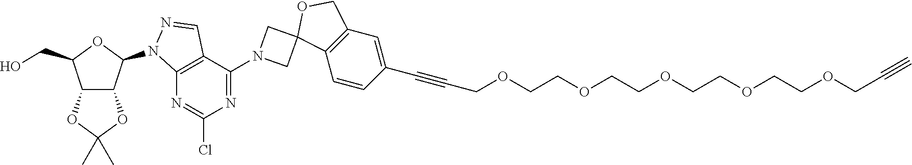 [SMILES: C#CCOCCOCCOCCOCCOCC#Cc1ccc2c(c1)COC21CN(c2nc(Cl)nc3c2cnn3[C@@H]2O[C@H](CO)[C@H]3OC(C)(C)O[C@H]32)C1]

To a solution of the triol (430 mg, 0.602 mmol) and 2,2-dimethoxypropane (188 mg, 1.81 mmol) in acetone (20 mL) at room temperature was added toluene sulfonic acid (103 mg, 0.602 mmol). The reaction was stirred for two hours then concentrated under reduced pressure. The crude material was dissolved in EtOAc (20 mL) and washed with saturated NaHCO₃. The organic layer was separated and dried over sodium sulfate, filtered, and concentrated in vacuo to provide crude acetonide derivative, which was purified by flash chromatography (DCM: 10% MeOH in DCM) to give a yellow solid. Yield 358 mg, 78.83%. Ion(s) found by LCMS: [M+H]⁺=754.2.

### Step e

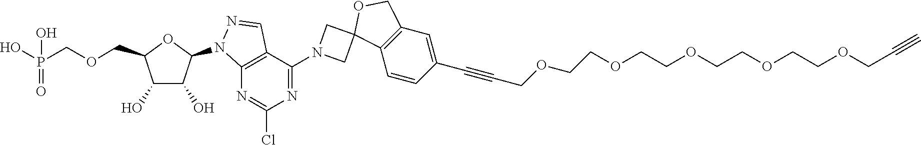 [SMILES: C#CCOCCOCCOCCOCCOCC#Cc1ccc2c(c1)COC21CN(c2nc(Cl)nc3c2cnn3[C@@H]2O[C@H](COCP(=O)(O)O)[C@@H](O)[C@H]2O)C1]

A solution of acetonide derivative from previous step (230 mg, 0.305 mmol) and [bis(tert-butoxy)carbonyl]methyl (trifluoromethyl)sulfonate (326 mg, 0.914 mmol) in dry DMF (5 mL) was cooled using ice-water bath. 60% NaH in mineral oil (74 mg, 1.83 mmol) was added to this and stirred at room temperature for 2 h. The reaction mixture was quenched with saturated ammonium chloride and extracted with EtOAc. The organic phase was washed with brine, dried over sodium sulfate, and concentrated to give crude product, which was used in the next step without purification. Ion(s) found by LCMS: [M+H]⁺=960.2. Crude product was dissolved in 50% aqueous TFA (10 mL) and stirred at room temperature for 12 h, concentrated and purified by semi-preparative HPLC (10 to 100% ACN/H₂O, 0.1% TFA modifier). The product was a white solid. Yield 185 mg, 75.07%. Ion(s) found by LCMS: [M+H]⁺=808.2.

### Step f

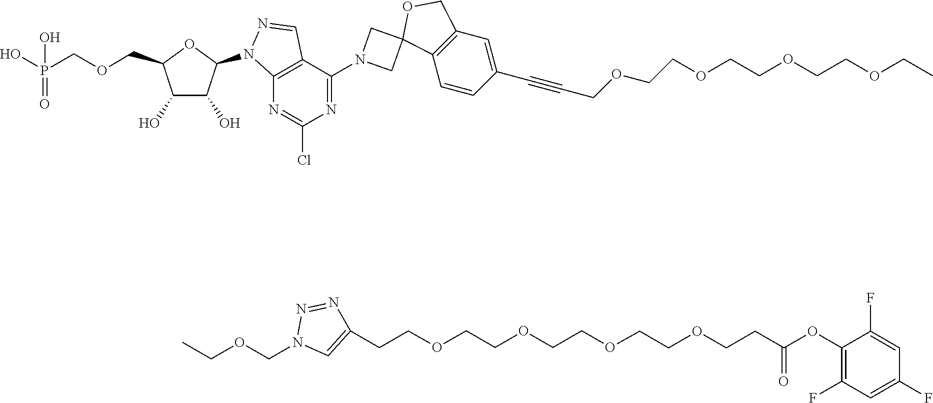

A solution of the product from the previous step (80 mg, 0.099 mmol) and intermediate A (42 mg, 0.0099 mmol) dissolved in DMF:H₂O (1:3, 1.5 mL), were cooled to 0° C. To this was added a pre-mixed solution of a solution of copper(II) sulfate (1.6 mg, 0.009 mmol), sodium ascorbate (59 mg, 0.297 mmol), and BTTA (8.5 mg, 0.098 mmol) dissolved in water (0.5 mL). When the reaction was complete (by HPLC), the mixture was quenched with a few drops of glacial acetic acid and 125 mM EDTA (pH 6) then purified by reversed phase HPLC (10% to 100% ACN/water, 0.1% TFA modifier). The product was a white solid. Yield 57 mg, 90.3%. Ion(s) found by LCMS [(M+2H)/2]⁺=576.2. The product was a white solid. Yield 102 mg, 83.81%. Ion(s) found by LCMS [(M+2H)/2]⁺=616.3.

### Synthesis of Conjugate 141a

Trifluorophenol ester (17 mg, 0.0137 mmol, Int-273) in DMF (0.5 mL) was added to Fc carrier SEQ ID NO: 13 (100 mg, 3.42 mL, 0.00172) in PBS at pH 7.4 as described in the conjugation procedure for Conjugate 131. Maldi TOF analysis of the purified final product gave an average mass of 62,089 (DAR=3.6). Yield 38.3 mg, 38.3%.

### Synthesis of Conjugate 141b

Trifluorophenol ester (22 mg, 0.0174 mmol, Int-273) in DMF (0.5 mL) was added to Fc carrier SEQ ID NO: 80 (120 mg, 8 mL, 0.0022) in PBS at pH 7.4 as described in the conjugation procedure for Conjugate 131. Maldi TOF analysis of the purified final product gave an average mass of 62462 Da. (DAR=6.7). Yield 55.3 mg, 46.1%.

### Synthesis of Int-275

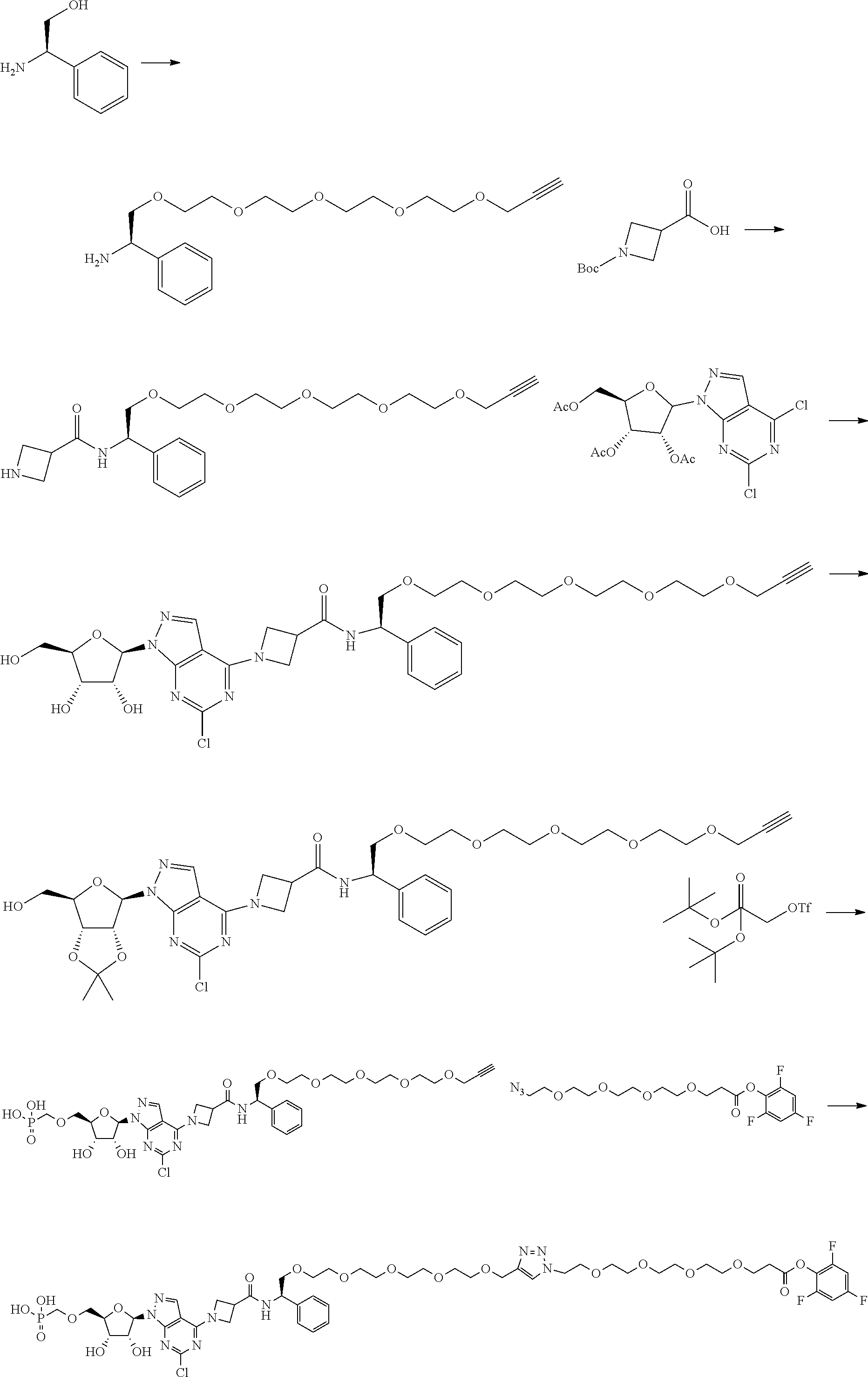

### Step a

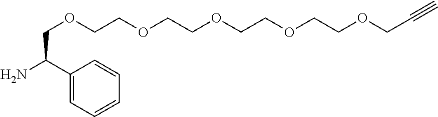 [SMILES: C#CCOCCOCCOCCOCCOC[C@H](N)c1ccccc1]

To a solution of R-phenylglycinol (500 mg, 3.65 mmol) in DMF (10 mL) at 0 C was added NaH (60% in mineral oil, 218 mg, 5.46 mmol), followed by propargylPEG4 mesylate (1.36 g, 4.37 mmol). The resulting mix heated at 70° C. overnight. The reaction mixture was cooled to room temperature and carefully quenched with water. The mixture was concentrated, and the residue was partitioned between EtOAc (50 mL) and water. The organic layer was washed with brine, dried over anhydrous sodium sulfate and concentrated. The residue was purified by RPLC (ACN: H₂O, 0.1% TFA modifier). The product was a yellow oil. Yield 1.2 g, 93.64%. Ion(s) found by LCMS [M+H]⁺=351.2.

### Step b

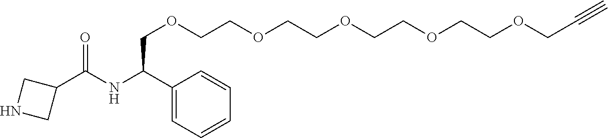 [SMILES: C#CCOCCOCCOCCOCCOC[C@H](NC(=O)C1CNC1)c1ccccc1]

N-Boc azetidine 3-carboxylic acid (500 mg, 2.48 mmol), HATU (945 mg, 2.48 mmol) and DIEA (0.86 mL, 4.97 mmol) in DMF (6 mL) were stirred together for 10 minutes at room temperature. The amine from the previous step (1 g, 2.98 mmol) in DMF (1 mL) was added, and the resulting mixture was stirred for 1 h. The reaction was concentrated and purified by semi-preparative HPLC (5% to 100% ACN/water). This gave the desired N-Boc protected product as a colorless viscous oil. (993 mg, 75%). Ion(s) found by LCMS: [M-tBu+H]+=479.2. N-Boc protected product (993 mg, 1.85 mmol) was dissolved in DCM (10 mL) and TFA (8 mL). The resulting mixture was stirred at room temperature for 3 h. Removal of the solvent followed by vacuum drying yielded the product. Yield 850 mg, quantitative. Ion(s) found by LCMS: [M+H]⁺=435.2.

### Step c

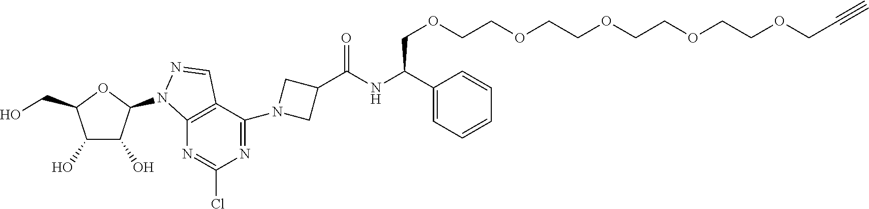 [SMILES: C#CCOCCOCCOCCOCCOC[C@H](NC(=O)C1CN(c2nc(Cl)nc3c2cnn3[C@@H]2O[C@H](CO)[C@@H](O)[C@H]2O)C1)c1ccccc1]

A mixture intermediate B (300 mg, 0.69 mmol), amine from previous step (292 mg, 0.83 mmol) and DIEA (0.24 mL, 1.38 mmol) in MeOH (6 mL, 1.12 mmol) were heated at 50° C. for 1 h. The mixture was cooled to room temperature and concentrated. The crude residue was dissolved in EtOAc (50 mL) and washed with water and brine. The organic layer was dried over sodium sulfate and solvent was removed under reduced pressure to yield triacetate derivative. Ion(s) found by LCMS: [M+H]⁺=845.2. The triacetate derivative in MeOH (5 mL) was treated with potassium carbonate (334 mg, 2.42 mmol) at room temperature for 2 h. The mixture was concentrated, dissolved in EtOAc (50 mL), and washed with water and brine. The organic layer was dried over sodium sulfate and solvent was removed under reduced pressure to yield the crude product which was further purified by n RPLC (ACN/water, 0.1% TFA modifier). The product was a yellow solid. Yield 420 mg, 84.33%. Ion(s) found by LCMS: [M+H]⁺=719.2.

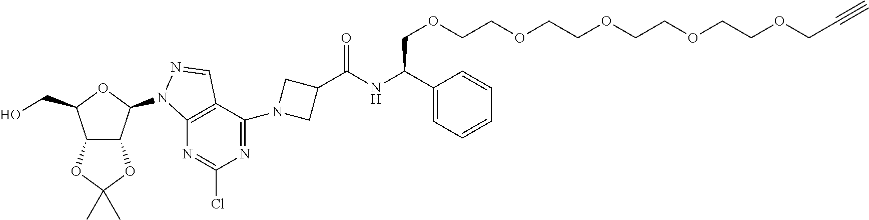 [SMILES: C#CCOCCOCCOCCOCCOC[C@H](NC(=O)C1CN(c2nc(Cl)nc3c2cnn3[C@@H]2O[C@H](CO)[C@H]3OC(C)(C)O[C@H]32)C1)c1ccccc1]

Triol derivative from previous step (420 mg, 0.584 mmol) and 2,2-dimethoxypropane (182 mg, 01.75 mmol) in acetone (4 mL) at room temperature was added p-TsOH (100 mg, 0.584 mmol). The reaction was stirred for two hours then concentrated under reduced pressure. The crude material was dissolved in ethyl acetate (20 mL) and washed with saturated sodium bicarbonate. The organic layer was separated and dried over sodium sulfate, filtered, and concentrated under reduced pressure to provide the acetonide derivative as an off-white solid. Yield 430 mg, 96.98%. Ion(s) found by LCMS: [M+H]+=759.2.

### Step e

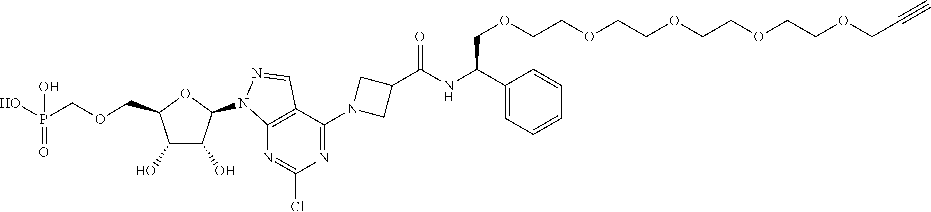 [SMILES: C#CCOCCOCCOCCOCCOC[C@H](NC(=O)C1CN(c2nc(Cl)nc3c2cnn3[C@@H]2O[C@H](COCP(=O)(O)O)[C@@H](O)[C@H]2O)C1)c1ccccc1]

Acetonide derivative (250 mg, 0.329 mmol) from the previous step, [bis(tert-butoxy)carbonyl]methyl (trifluoromethyl)sulfonate (352 mg, 988 mmol), and 60% NaH in mineral oil (79 mg, 1.98 mmol) was added at room temperature then stirred for 2 h. The reaction mixture was quenched with saturated ammonium chloride and extracted with EtOAc. The organic phase was washed with brine, dried over sodium sulfate, concentrated to give crude product, which was used in the next step without purification. Ion(s) found by LCMS: [M+H]⁺=965.2. The crude product was dissolved in 50% aqueous TFA (10 mL) and stirred at room temperature for 12 h, then concentrated and purified by semi-preparative HPLC (10 to 100% ACN/H₂O, 0.1% TFA modifier). The product was a white solid. Yield 217 mg, 81%. Ion(s) found by LCMS: [M+H]⁺=813.2.

### Step f

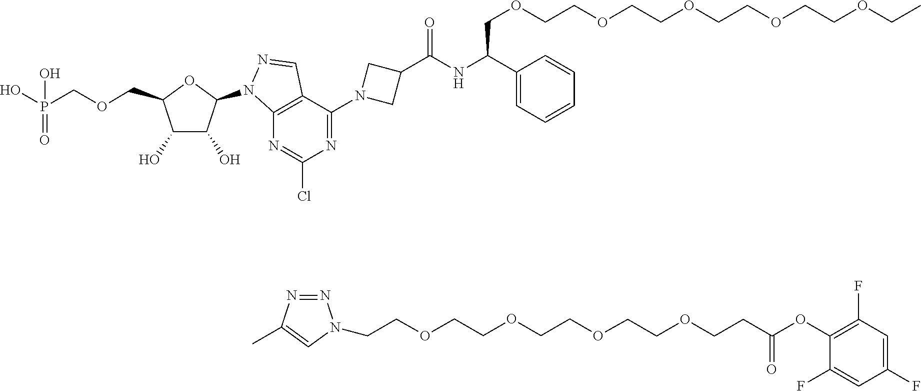

To a solution of the product from the previous step (21 mg, 0.049 mmol) and intermediate A (23 mg, 0.049 mmol) dissolved in DMF:H₂O (1:3, 1.5 mL) were cooled to 0° C. To this a pre-mixed solution of a solution of copper(II) sulfate (0.78 mg, 0.0049 mmol), sodium ascorbate (29 mg, 0.147 mmol), and BTTA (4.2 mg, 0.0098 mmol) dissolved in water (0.5 mL) was added and stirred for 5 minutes at the same temperature then gradually warmed to room temperature and stirred for 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 32 mg, 52.7%. Ion(s) found by LCMS [(M+2H)/2]⁺=617.8.

### Synthesis of Conjugate 142

Trifluorophenol ester (13 mg, 0.0101 mmol, Int-275) in DMF (0.5 mL) was added to Fc carrier SEQ ID NO: 80 (70 mg, 4.67 mL, 0.00126 mmol) in PBS at pH 7.4 as described in the conjugation procedure for Conjugate 71. Maldi TOF analysis of the purified final product gave an average mass of 61,885 (DAR=6.1). Yield 31.9 mg, 45.6%.

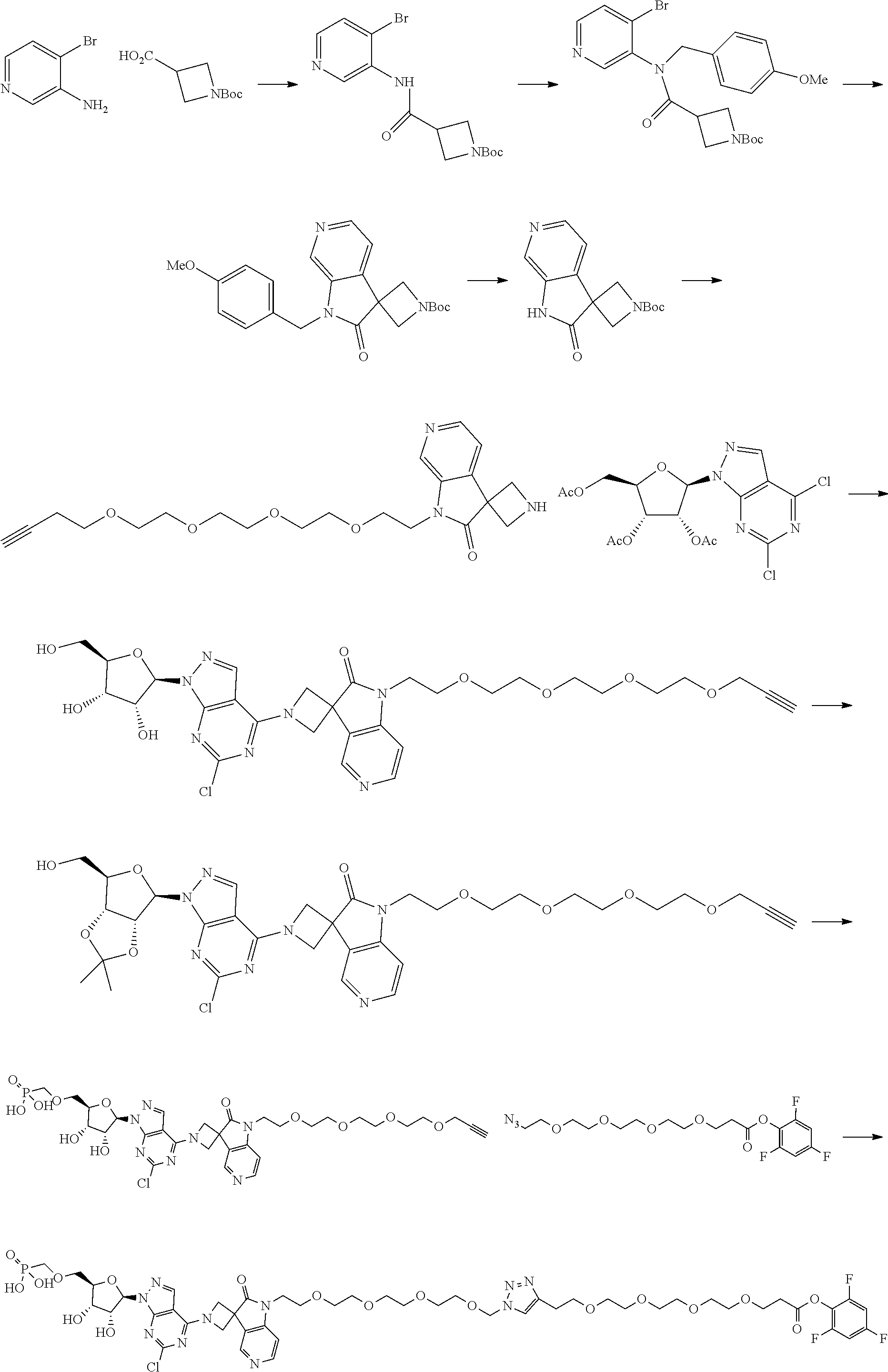

### Synthesis of Int-277

### Step a

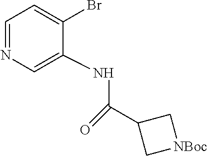 [SMILES: CC(C)(C)OC(=O)N1CC(C(=O)Nc2cnccc2Br)C1]

To a solution of 3-amino-4-bromopyridine 5 g, 28.9 mmol) and N-Boc-azetidine ˜3-carboxylic acid (6.1 g, 1.05 mmol) in dry DCM (100 mL) was added DMAP (4.59 g, 37.57 mmol) and EDCI (5.83 g, 37.57). The resulting mixture was stirred at room temperature for 48 h, then diluted with ethyl acetate and washed with water and brine. The aqueous phase was extracted with ethyl acetate, dried over sodium sulfate, and purified by normal phase silica column using DCM to 10% MeOH/DCM. The product was an off white solid. Yield 9.8 g, 95.1%. Ion(s) found by LCMS [M+H]⁺=356.0.

### Step b

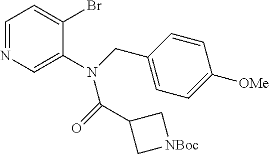 [SMILES: COc1ccc(CN(C(=O)C2CN(C(=O)OC(C)(C)C)C2)c2cnccc2Br)cc1]

To a solution of the compound from previous step (7.12 g, 19.99 mmol) in DMF (25 mL) was added Cs₂CO₃ (16.3 g, 49.97 mmol) and 4-methoxy benzyl chloride (7.12 g, 45.97 mmol) in benzene (10 mL). The reaction was stirred for 2 h at room temperature, water was added, and the mixture was extracted with EtOAc (3×). The organic phase was washed with brine, and dried over Na₂sO₄. Removal of the solvent, followed by normal phase column chromatography using DCM: EtOAc yielded the desired compound, which was an off white solid. Trituration with hexanes gave the product as a white solid. Yield 8.7 g, 91.37%. Ion(s) found by LCMS [M-tBu+H]+=420.2.

### Step c

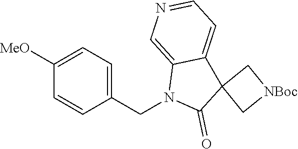 [SMILES: COc1ccc(CN2C(=O)C3(CN(C(=O)OC(C)(C)C)C3)c3ccncc32)cc1]

To a solution of the compound from previous (3 g, 6.3 mmol) in dioxane (50 mL) were added tricyclohexylphosphine (176 mg, 0.63 mmol), Pd(OAc)₂ (141 mg, 0.63 mmol) and sodium tert-butoxide (907 mg, 9.45 mmol) under nitrogen. The resulting mixture was heated at 95° C. for 2 h. The reaction mixture was cooled to room temperature, quenched with ammonium chloride, and extracted with DCM (3×). The combined organic extracts were washed with brine, dried over anhydrous sodium sulfate, purified by normal phase silica column chromatography (DCM: EtOAc). The product was a pale yellow solid. Yield 1.67 g, 67.06%. Ion(s) found by LCMS [M+H]⁺=396.2.

### Step d

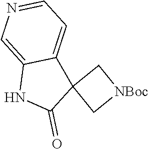 [SMILES: CC(C)(C)OC(=O)N1CC2(C1)C(=O)Nc1cnccc12]

To a solution of product from the previous step (1.3 g, 3.28 mmol) dissolved in ACN (30 mL) at 0° C. was added a solution of cerium (IV)ammonium nitrate (3.60 g, 6.57 mmol) in water (5 mL). The resulting mixture was stirred at room temperature for 5 h. The reaction mixture was quenched with 5% potassium carbonate solution, and then extracted with EtOAc (3×). The combined organic extracts were washed with brine and dried over anhydrous sodium sulfate. Removal of the solvent followed by normal phase silica column chromatography (DCM to 10% MeOH in DCM) yielded desired product as an off white solid, 450 mg, 49.72%. Ion(s) found by LCMS [M+H]⁺=276.2.

### Step e

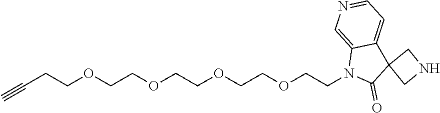 [SMILES: C#CCCOCCOCCOCCOCCN1C(=O)C2(CNC2)c2ccncc21]

Amine from the previous step (507 mg, 1.63 mmol), propargyl PEG₄mesylate (450 mg, 1.63 mmol) and Cs₂CO₃ (1.1 g, 3.27 mmol) were dissolved in ACN (20 mL) and heated at 90° C. for 6 h. The reaction mixture was cooled to room temperature, filtered, and washed with ethyl acetate. The filtrate was washed with water, brine, and dried over sodium sulfate. Removal of the solvent yielded crude compound as a reddish orange oil, which was purified by semi-preparative HPLC (10-100% ACN/water, 0.1% TFA modifier). The product was a yellow oil. Yield 390 mg, 49%. Ion(s) found by LCMS [M+H]⁺=490.2. N-Boc protected compound (390 mg, 0.79 mmol) was dissolved in DCM (5 mL) and TFA (5 mL) then stirred at room temperature for 2 hours. Solvent was removed under rotary evaporation and vacuum dried to get the desired compound as an off white solid. Yield 320 mg, 100%. Ion(s) found by LCMS [M+H]⁺=390.2.

### Step f

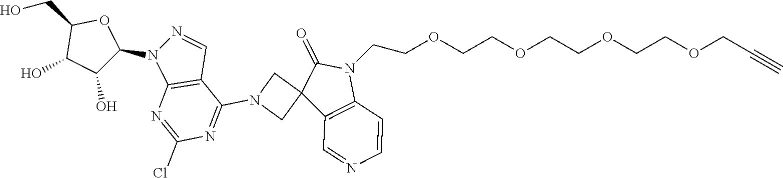 [SMILES: C#CCOCCOCCOCCOCCN1C(=O)C2(CN(c3nc(Cl)nc4c3cnn4[C@@H]3O[C@H](CO)[C@@H](O)[C@H]3O)C2)c2cnccc21]

A mixture of intermediate B (300 mg, 0.671 mmol), amine from previous step (314 mg, 0.805 mmol), and DIEA (0.35 mL) in MeOH (10 mL) was heated at 50° C. for 1 h. After consumption of the starting materials, the reaction mixture was cooled to room temperature and volatiles were removed by rotatory evaporation. The crude product was dissolved in EtOAc (50 mL) and washed with water and brine. The organic layer was dried over sodium sulfate and concentrated to yield a yellow foam which was used in the next step without further purification. Ion(s) found by LCMS: [M+H]⁺=800.2. The triacetate intermediate was dissolved in MeOH (6 mL), treated with potassium carbonate (324 mg, 2.35 mmol), and stirred at room temperature for 2 h. The crude reaction was filtered through a short pad of celite and washed with methanol and concentrated. Crude material obtained was then purified by semi-preparative HPLC (5% to 100% ACN/water, 0.1% TFA modifier) to yield the desired product as an off-white foam. Yield 250 mg, 55.25%. Ion(s) found by LCMS: [M+H]⁺=674.2

### Step g

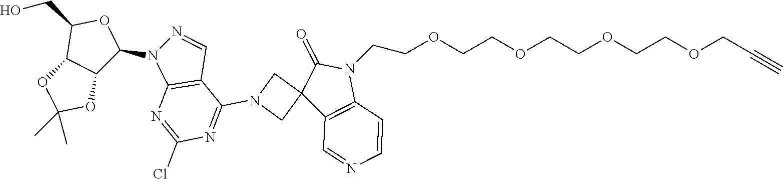 [SMILES: C#CCOCCOCCOCCOCCN1C(=O)C2(CN(c3nc(Cl)nc4c3cnn4[C@@H]3O[C@H](CO)[C@H]4OC(C)(C)O[C@H]43)C2)c2cnccc21]

To a solution of the triol from previous step (250 mg, 0.371 mmol) and 2,2-dimethoxypropane (116 mg, 1.11 mmol) in acetone (10 mL) at room temperature was added toluene sulfonic acid (70 mg, 0.371 mmol). The reaction was stirred for two hours and then concentrated under reduced pressure. The crude material was dissolved in EtOAc (20 mL), washed with saturated NaHCO₃, dried over sodium sulfate, filtered, and concentrated in vacuo to provide crude acetonide derivative, which was purified by flash chromatography (DCM: 10% MeOH in DCM). The desired product was a yellow solid. Yield 215 mg, 81.1%. Ion(s) found by LCMS: [M+H]⁺=714.2.

### Step h

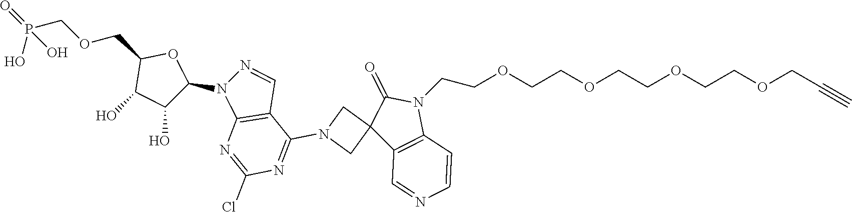 [SMILES: C#CCOCCOCCOCCOCCN1C(=O)C2(CN(c3nc(Cl)nc4c3cnn4[C@@H]3O[C@H](COCP(=O)(O)O)[C@@H](O)[C@H]3O)C2)c2cnccc21]

A solution of acetonide derivative from previous step (215 mg, 0.30 mmol) and [bis(tert-butoxy)carbonyl]methyl (trifluoromethyl)sulfonate (326 mg, 0.914 mmol) in dry DMF (5 mL) was cooled using ice-water bath. To this solution was added 60% NaH in mineral oil (74 mg, 1.83 mmol). The mixture was stirred at room temperature for 2 h., then quenched with saturated ammonium chloride and extracted with EtOAc. The organic phase was washed with brine, dried over sodium sulfate, and concentrated to give the crude product, which was used in the next step without purification. Ion(s) found by LCMS: [M+H]⁺=920.4. Crude product was dissolved in 50% aqueous TFA (10 mL) and stirred at room temperature for 2 h, then concentrated and purified by semi-preparative HPLC (10-100% ACN: H₂O, 0.1% TFA modifier). The product was a white solid. Yield 124 mg, 53.9%. Ion(s) found by LCMS: [M+H]⁺=768.2.

### Step i

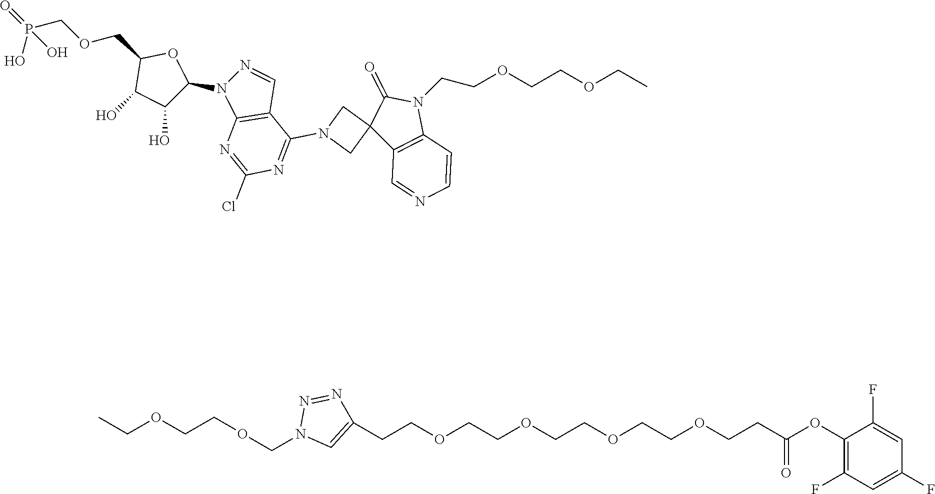

To a solution of the product from the previous step (60 mg, 0.078 mmol) and intermediate (33 mg, 0.078 mmol), dissolved in DMF:H₂O (1:3, 1.5 mL) were cooled to 0° C. A pre-mixed solution of a solution of copper(II) sulfate (1.2 mg, 0.008 mmol), sodium ascorbate (46 mg, 0.234 mmol), and BTTA (6.7 mg, 0.0156 mmol) dissolved in water (0.5 mL) was added and then stirred for 5 minutes and gradually warmed to room temperature and stirred room temperature for 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 51.8 mg, 55.75%. Ion(s) found by LCMS [M+H]⁺=1191.2.

### Synthesis of Conjugate 143

Trifluorophenol ester (14 mg, 0.0116 mmol, Int-277) in DMF (0.5 mL) was added to Fc carrier 231 (80 mg in 5.33 mL PBS 7.4, 0.00148 mmol) in PBS at pH 7.4 as described in the conjugation procedure for Conjugate 71. Maldi TOF analysis of the purified final product gave an average mass of 58,740 Da. (DAR=3.4). Yield 39.1 mg, 38.8%.

### Synthesis of Int-252

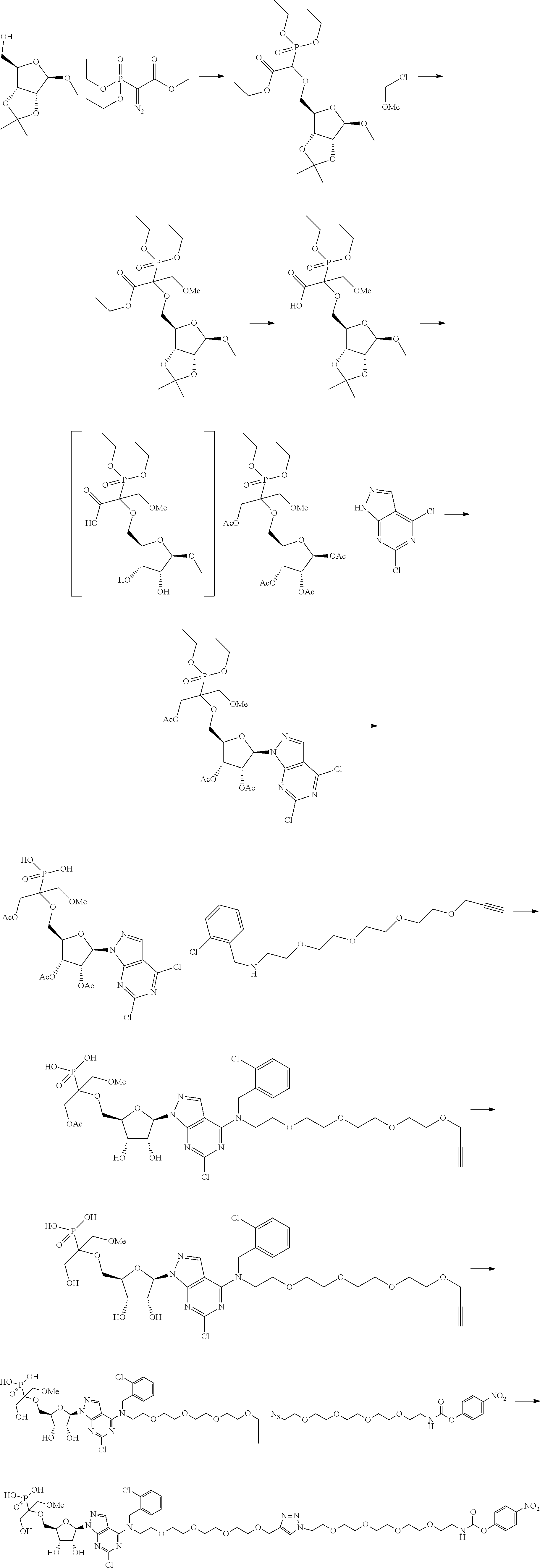

### Step a

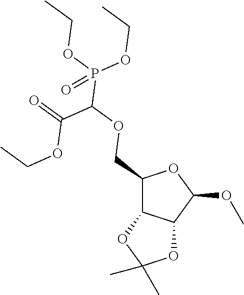 [SMILES: CCOC(=O)C(OC[C@H]1O[C@@H](OC)[C@@H]2OC(C)(C)O[C@@H]21)P(=O)(OCC)OCC]

Methyl-2,3-o-isopropylidene-beta-D-ribofuranoside (2 g, 9.8 mmol), ethyl-2-diazo-2-(diethylphosphono) acetate (1 g, 4 mmol), and benzene were added to a sealed tube and evacuated and flushed with nitrogen (3×). Rhodium tetraacetate (125 mg, 0.28 mmol was added and the tube was again evacuated and flushed with nitrogen (3×). The tube was sealed and stirred at 10000 for 16 hours. The reaction was cooled to ambient temperature, filtered through celite and concentrated. The crude reaction mixture was purified by silica gel chromatography (0-75% ethyl acetate in hexanes). The pure fraction(s) were pooled and concentrated to afford the intermediate as a clear oil. Yield 1.2 grams, 71%. Ion(s) found by LCMS: [M+Na]⁺=449.2.

### Step b

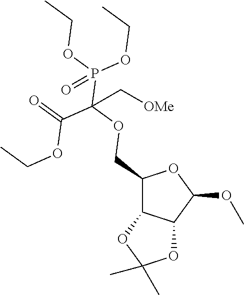 [SMILES: CCOC(=O)C(COC)(OC[C@H]1O[C@@H](OC)[C@@H]2OC(C)(C)O[C@@H]21)P(=O)(OCC)OCC]

The product of the previous step (1 g, 2.3 mmol) was dissolved in anhydrous DMF (15 mL) and cooled to 0° C. via an ice/water bath. Sodium HMDS (2.5 mL, 2.5 mmol, 1M in THF) was added and the mixture was stirred for 10 minutes followed by the dropwise addition of MOM Chloride (0.35 mL, 4.6 mmol). The ice bath was removed, and the reaction was stirred for an additional 20 minutes then cooled to 0° C. and quenched with the dropwise addition of saturated aqueous ammonium chloride solution (˜50 mL). The mixture was extracted into ethyl acetate (3×, 20 mL). The combined organic extracts were washed with brine, dried over sodium sulfate, and concentrated. The crude reaction mixture was purified by silica gel chromatography (0-100% ethyl acetate in hexanes). The pure fraction(s) were pooled and concentrated to afford the intermediate as a clear oil, mixture of diastereomers. Yield 0.82 grams, 75%. Ion(s) found by LCMS: [M+Na]⁺=493.2.

### Step c

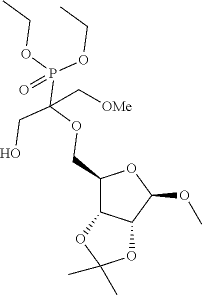 [SMILES: CCOP(=O)(OCC)C(CO)(COC)OC[C@H]1O[C@@H](OC)[C@@H]2OC(C)(C)O[C@@H]21]

Product from the previous step (0.82 g, 1.7 mmol) was dissolved in ethanol (50 mL). Calcium chloride powder (556 mg, 5.1 mmol) and sodium borohydride (189 mg, 5.1 mmol) were added, and the reaction was stirred at ambient temperature for 4 hours. The mixture was filtered through celite and treated with ˜2 mL of glacial acetic acid. The mixture was concentrated then diluted with saturated aqueous ammonium chloride solution 100 mL, extracted into ethyl acetate (3×, 40 mL). The combined extracts were dried over sodium sulfate and concentrated. The crude residue was purified by silica gel chromatography (DCM/methanol) to afford the intermediate as a clear oil. Yield 0.62 grams, 85%. Ion(s) found by LCMS: [M−OMe]⁺=397.2, LCMS: [M+Na]⁺=451.2.

### Step d

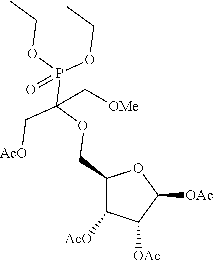

The product of the previous step (620 mg, 1.4 mmol) was stirred in a 70% solution of aqueous acetic acid at 90° C. for 6 hours. The mixture was concentrated to dryness on the rotary evaporator and then dried under high vacuum for 12 hours. The residue was dissolved in DCM (25 mL) then DIPEA (3.6 g, 28 mmol) was added to the stirring mixture, followed by acetic anhydride (1.4 g, 14 mmol). The reaction was stirred for 4 hours then concentrated on the rotary evaporator. The crude residue was purified by silica gel chromatography (DCM/Methanol) to afford the intermediate as a clear oil. Yield 0.22 grams, 30%. Ion(s) found by LCMS: [M+Na]⁺=565.2.

### Step e

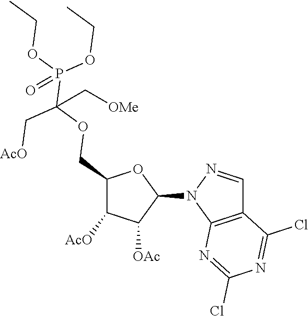

4,6-Dichloro-1H-pyrazolo[3,4,d]pyrimidine (80 mg, 0.42 mmol) and ammonium sulfate (˜3 mg) were dissolved in 25 mL of hexamethyldisilzane. The mixture was then heated to 130° C. and stirred for 3 hours. The mixture was then concentrated on the rotary evaporator and died under high vacuum for 12 hours. The solid residue was then taken up in 100 mL of acetonitrile, and the product from step d of this example (220 mg, 0.39 mmol) was added, and the mixture was stirred until all solids were dissolved. The mixture was cooled to 0° C., and TMSOTf (100 uL, 0.5 mmol) was added. The reaction mixture was gradually warmed to ambient temperature and allowed to stir for 3 hours. The mixture was concentrated and taken up in ethyl acetate (50 mL). The organic extract was washed with saturated sodium bicarbonate, then brine, dried over sodium sulfate, filtered, and concentrated. The crude residue was purified reversed phase HPLC (5-85% acetonitrile in DI water, 25 minute gradient, 0.1% TFA modifier).

Yield 136 mg, 53%. Ion(s) found by LCMS: [M+Na]⁺=693.4.

### Step f

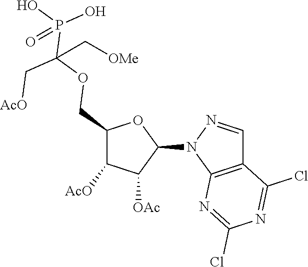

The product from step e. of this example (115 mg, 0.17 mmol) was dissolved in acetonitrile (8 mL). DIEA (658 mg, 5.1 mmol) was added followed by TMS bromide (520 mg, 3.4 mmol). The reaction was stirred at ambient temperature for 30 minutes, then concentrated on the rotovap and purified by reversed phase HPLC (5-85% acetonitrile in DI water, 25 minute gradient, 0.1% TFA modifier). The pure fraction(s) were pooled and lyophilized. Yield 33 mg, 32%. Ion(s) found by LCMS: [M+H]⁺=615.2.

### Step g

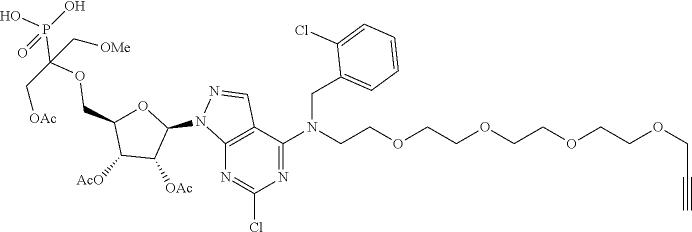 [SMILES: C#CCOCCOCCOCCOCCN(Cc1ccccc1Cl)c1nc(Cl)nc2c1cnn2[C@@H]1O[C@H](COC(COC)(COC(C)=O)P(=O)(O)O)[C@@H](OC(C)=O)[C@H]1OC(C)=O]

The product from step h of this example (136 mg, 0.22 mmol), DIEA (64 mg, 0.50 mmol) and the amine from Int-22 (106 mg, 0.30 mmol) were stirred together in ethanol (20 ml) at 50° C. for 45 minutes. The mixture was concentrated on the rotovap and purified by reversed phase HPLC (5-85% acetonitrile in DI water, 25 minute gradient, 0.1% TFA modifier). Yield 158 mg, 77%. Ion(s) found by LCMS: [M+H]⁺=934.6.

### Step h

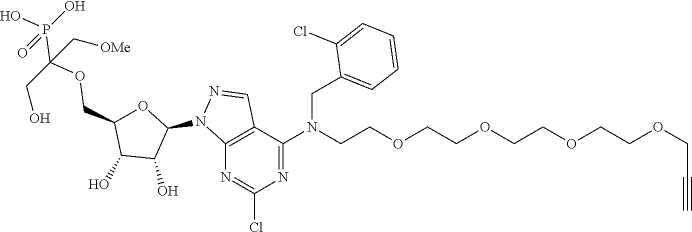 [SMILES: C#CCOCCOCCOCCOCCN(Cc1ccccc1Cl)c1nc(Cl)nc2c1cnn2[C@@H]1O[C@H](COC(CO)(COC)P(=O)(O)O)[C@@H](O)[C@H]1O]

The product from step g. of this example (35 mg, 0.038 mmol) was stirred in methanol containing 15 mg of potassium carbonate powder at ambient temperature for 45 minutes. The mixture was filtered and 2 mL of glacial acetic acid was added. The mixture was concentrated and purified by reversed phase HPLC (5-85% acetonitrile in DI water, 25 minute gradient, 0.1% TFA modifier). The pure fraction(s) were pooled and lyophilized. Yield 22 mg, 71%. Ion(s) found by LCMS: [M+H]⁺=808.2.

### Step i

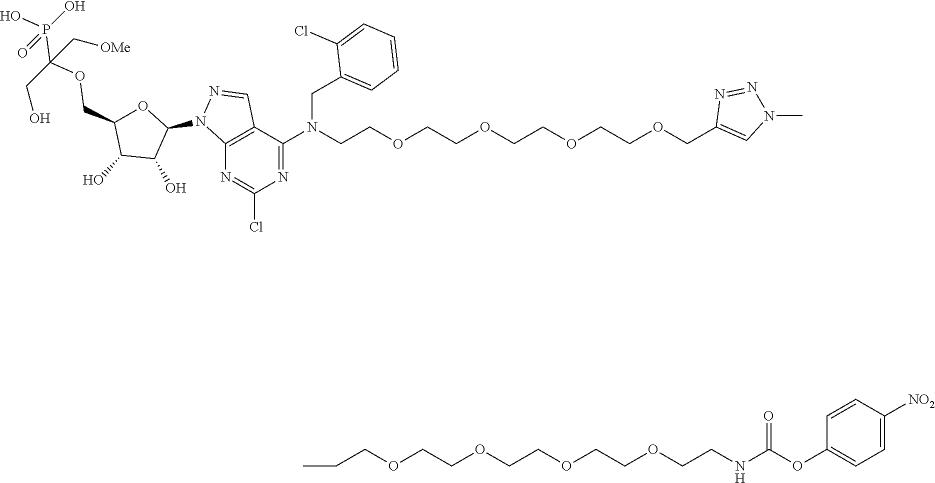

The product from step h of this example (22 mg, 0.017 mmol) and azido-peg₄-p-nitrophenol ester (15 mg, 0.035 mmol) were dissolved in DMF (1 mL) and added to a mixture of sodium ascorbate (16 mg, 0.082 mmol), BTTA (3 mg, 0.0068 mmol) and copper sulfate (6 mg, 0.0041 mmol) in DI water and the mixture was stirred for 30 minutes. The mixture was filtered and purified by reversed phase HPLC (5-95% acetonitrile in DI water, 25 minute gradient, 0.1% TFA modifier). Yield 25 mg, 74%. Ion(s) found by LC/MS [(M+H]+=1235.2.

### Synthesis of Conjugate 128

The title compound was prepared analogously to Conjugate 4a, where Int-110 was replaced by Int-252 and azido-peg₄-trifluorophenol ester was replaced with azido-peg₄-p-nitrophenol ester (prepared as described in the synthesis of N-[2-(2-{2-[2-(2-azidoethoxy)ethoxy]ethoxy}ethoxy)ethyl](4-nitrophenoxy)carboxamide). Maldi TOF analysis of the purified final product gave an average mass of 59,903 Da (DAR=3.4).

### Synthesis of Int-278

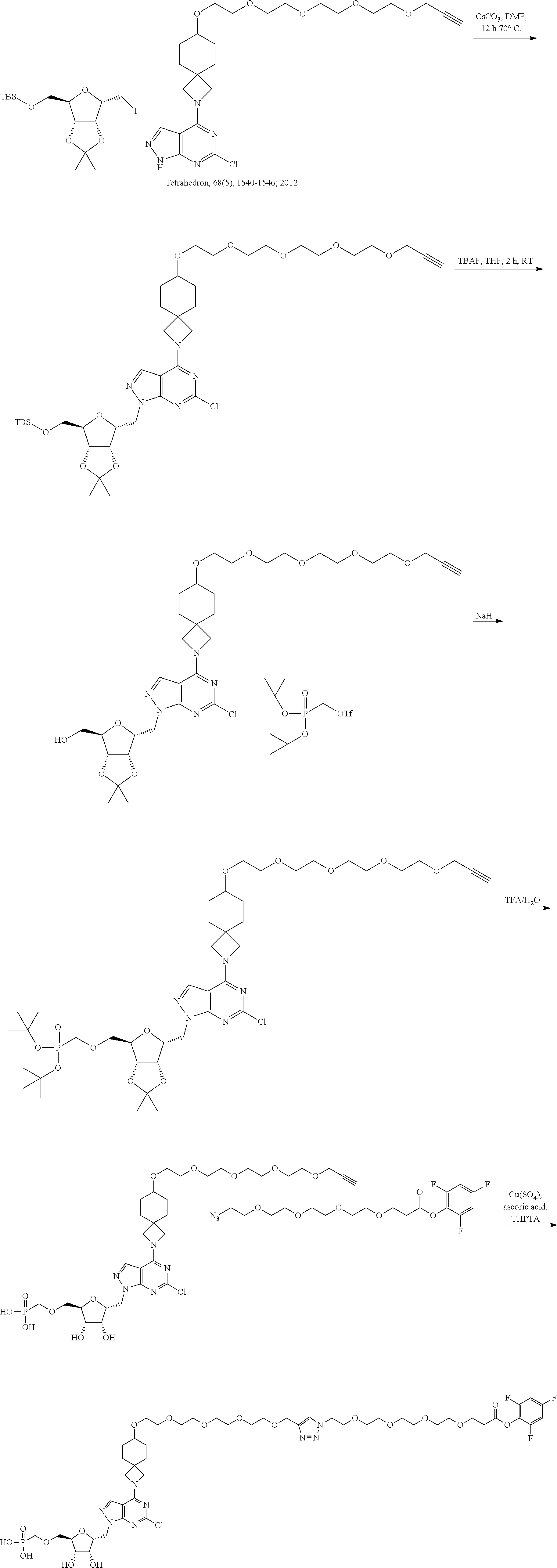

Int-278 is prepared according to the above synthetic scheme, methods provided herein, and methods known to those of skill in the art.

### Synthesis of Int-286

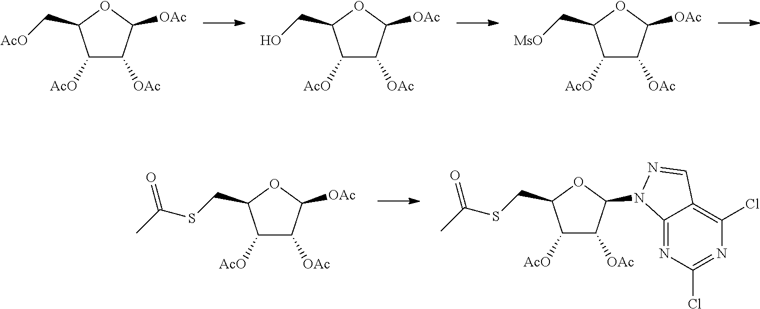 [SMILES: CC(=O)OC[C@H]1O[C@@H](OC(C)=O)[C@H](OC(C)=O)[C@@H]1OC(C)=O ; CC(=O)O[C@@H]1O[C@H](CO)[C@@H](OC(C)=O)[C@H]1OC(C)=O ; CC(=O)O[C@@H]1O[C@H](COS(C)(=O)=O)[C@@H](OC(C)=O)[C@H]1OC(C)=O ; CC(=O)O[C@@H]1O[C@H](CSC(C)=O)[C@@H](OC(C)=O)[C@H]1OC(C)=O ; CC(=O)O[C@@H]1[C@H](OC(C)=O)[C@@H](CSC(C)=O)O[C@H]1n1ncc2c(Cl)nc(Cl)nc21]

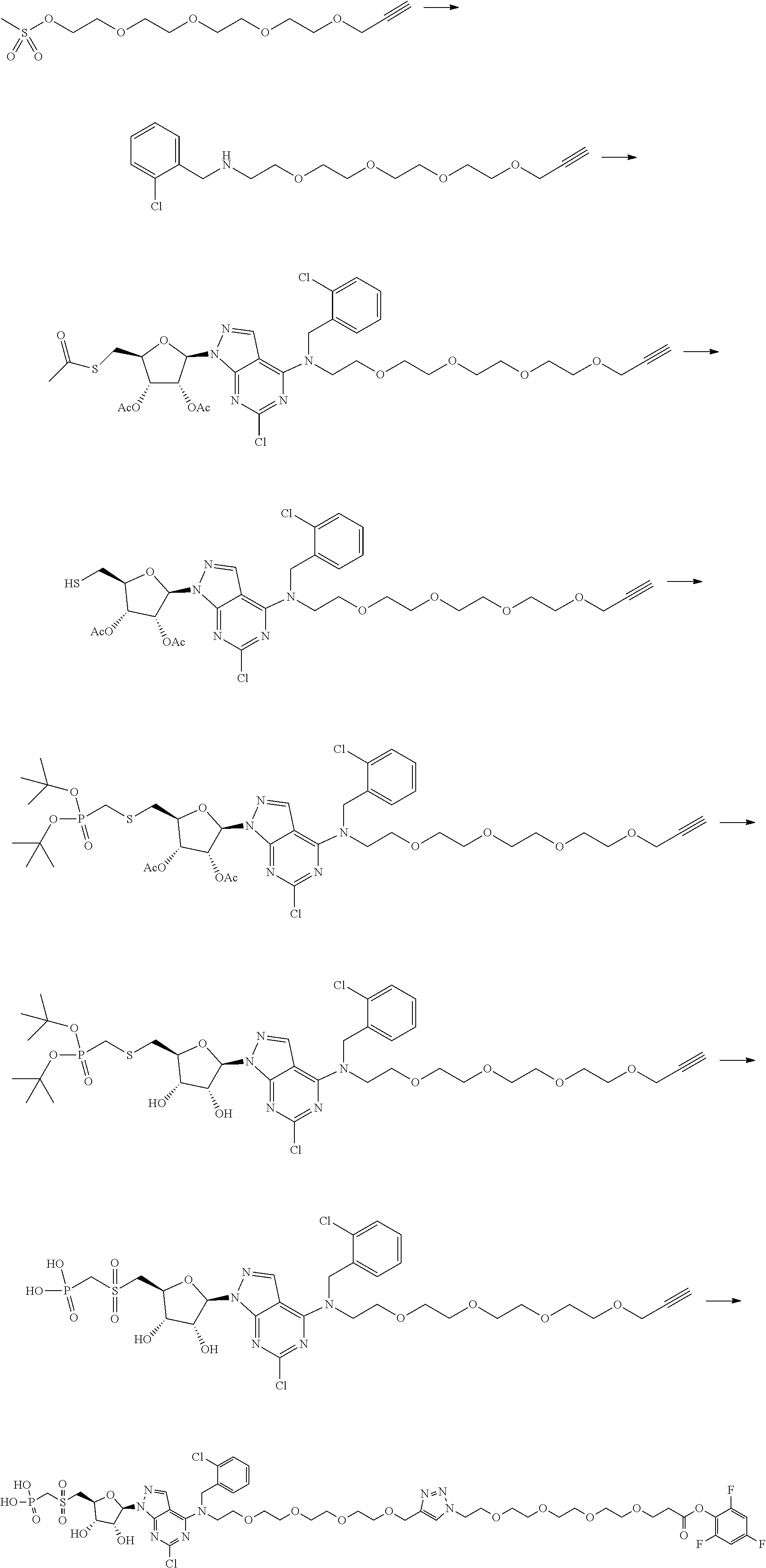

### Step a

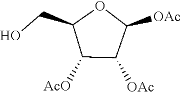

To a solution of beta-D-ribofuranose 1,2,3-5-tetraacetate (31.83 g, 100 mmol) in a mixture of DMF (20 ml) and acetonitrile (40 ml) was added 250 ml of PBS 7.4 buffer (10×) and lipase from Candida rugosa, type VII, 700 unit/mg solid (25 g). The resulting mixture was gently stirred for 2 hours, then addition PBS 7.4 buffer (50 ml) and NaHCO₃ (4.2 g, 50 mmol) were added. The reaction was gently stirred for overnight. It was then poured into a separatory funnel containing EtOAc (300 ml) and hexane (50 ml). The layers were separated, and the aqueous layer was gently extracted with EtOAc (300 ml). The combined organic layers contaminated with some aggregated protein were dried over Na₂SO₄, concentrated by rotary evaporation, and purified through silica gel column chromatography (120 g, 0 to 50% EtOAc and hexane). Yield 10.3 g, 37.3%. Ions found by LCMS: [M+Na]⁺=299.0, [M−AcOH]⁺=217.0.

### Step b

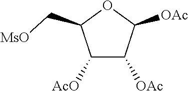

Product from step a (8.07 g, 29.2 mmol) was dissolved in DCM (50 ml), and the solution was cooled in an ice-water bath. DIPEA (7.55 g, 58.4 mmol) was added, followed by a slow addition of methanesulfonyl chloride (5.02 g, 43.8 mmol). The ice-water bath was then removed, and the reaction mixture was stirred for 2 hours. It was then extracted with water (50 ml) and washed successively with 5% NaHCO₃ in water (30 ml×2) and then water (30 ml). The organic layer was dried over Na₂SO₄, concentrated by rotary evaporation, and further dried under high vacuum. The crude product was carried to the subsequent step without purification. Yield 9.89 g, 95.6%. Ions found by LMS: [M+Na]⁺=377.0, [M−AcOH]⁺=295.0.

### Step c

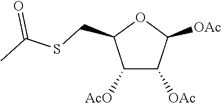

A reaction flask equipped with a strong stirrer was filled with nitrogen and charged with product from step b (8.58 g, 24.21 mmol), anhydrous DMF (28 ml), anhydrous acetonitrile (56 ml), KI (2.32 g, 14 mmol), and potassium thioacetate (3.19 g, 27.9 mmol). The resulting mixture was well stirred at 70° C. under nitrogen for 1 hour. DIPEA (1.81 g, 14 mmol) and addition potassium thioacetate (2.4 g, 8.76 mmol) were added, and the reaction was continued overnight. After cooling to room temperature, the solid was filtered through celite and washed with EtOAc. The filtrate was concentrated by rotary evaporation. The residue was diluted with EtOAc:hexane (3:1, 150 ml) and successively washed with water (50 ml), 5% NaHCO₃ (30 ml×2) and then again with water (50 ml×2). The organic layer was concentrated by rotary evaporation and purified through silica gel column chromatography (120 g, 0 to 30% EtOAc and hexane). Yield 7.32 g, 78.5%. Ions found by LCMS: [M+Na]⁺=357.0, [M−AcOH]⁺=275.0.

### Step d

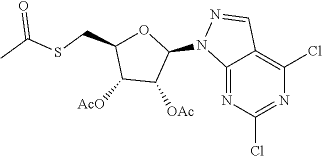 [SMILES: CC(=O)O[C@@H]1[C@H](OC(C)=O)[C@@H](CSC(C)=O)O[C@H]1n1ncc2c(Cl)nc(Cl)nc21]

A flame-dried reaction flaks was filled with nitrogen and charged with product from step c (3.46 g, 10.35 mmol), 1,2-dichloroethane (20 ml), and 4,6-dichloro-1H-pyrazolo[3,4-d]pyrimidine (3.67 g, 9.44 mmol). The suspension mixture was slowly added with Tin(IV) chloride (0.3 ml, 2.56 mmol). After stirring for 1.5 hours, the reaction mixture was slowly quenched with water (20 ml). The layers were separated, and the aqueous layer was back-extracted with DCM (20 ml). The combined organic layers were concentrated by rotary evaporation and purified through silica gel column chromatography: (80 g, 0 to 50% EtOAc and hexane). Yield 3.27 g, 68.2%. Ion found by LMS: [M+Na]⁺=485.0.

### Step e

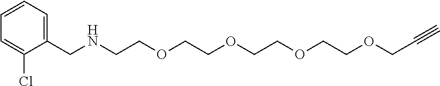 [SMILES: C#CCOCCOCCOCCOCCNCc1ccccc1Cl]

To a solution of 2-chlorobenzylamine (1.4 g, 7.77 mmol) in acetonitrile (15 ml) was added K₂CO₃ (967.4 mg, 7 mmol) and propargyl-PEG4-mesyl ester (2.18 g, 7 mmol). The resulting mixture was heated at 70° C. overnight. The salt was filtered off and washed with ACN. The filtrate was concentrated by rotary evaporation and purified through RPLC (100 g, 5 to 80% acetonitrile and water) Yield 905.7 mg, 36.4%. Ion found by LCMS: [M+H]⁺=356.2.

### Step f

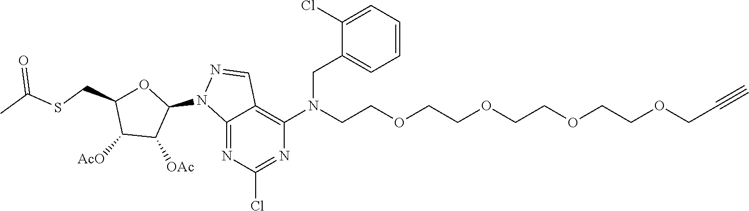 [SMILES: C#CCOCCOCCOCCOCCN(Cc1ccccc1Cl)c1nc(Cl)nc2c1cnn2[C@@H]1O[C@H](CSC(C)=O)[C@@H](OC(C)=O)[C@H]1OC(C)=O]

A solution of product from step d (184.7 mg, 0.399 mmol), product from step e (156 mg, 0.332 mmol), and DIPEA (245.5 mg, 1.9 mmol) in anhydrous DMF (0.5 ml) was stirred at room temperature for 50 minutes. It was then directly purified through RPLC (100 g, 5 to 100% acetonitrile and water). Yield 142.4 mg, 54.8%. Ions found by LCMS: [M+Na]⁺=804.0, [M+H]⁺=782.2.

### Step g

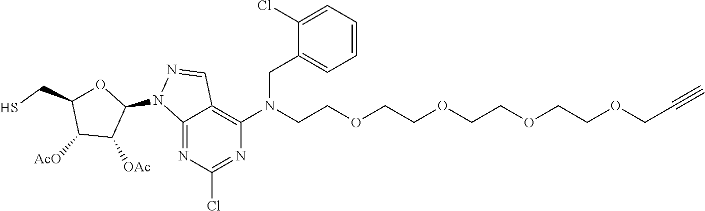 [SMILES: C#CCOCCOCCOCCOCCN(Cc1ccccc1Cl)c1nc(Cl)nc2c1cnn2[C@@H]1O[C@H](CS)[C@@H](OC(C)=O)[C@H]1OC(C)=O]

A reaction flask was filled with nitrogen and charged with product from step f (142.4 mg, 0.182 mmol), MeOH (2.5 ml), Et₃N (184 mg, 1.8 mmol), and DL-dithiothreitol (28.1 mg, 0.182 mmol). The resulting mixture was stirred at room temperature for 1.5 hours. It was then concentrated by rotary evaporation and purified through silica gel column chromatography (40 g, 0 to 100% EtOAc and hexane). Yield 94.4 mg 70%. Ions found by LCMS: [M+Na]⁺=762.0, [M+H]⁺=740.2.

### Step h

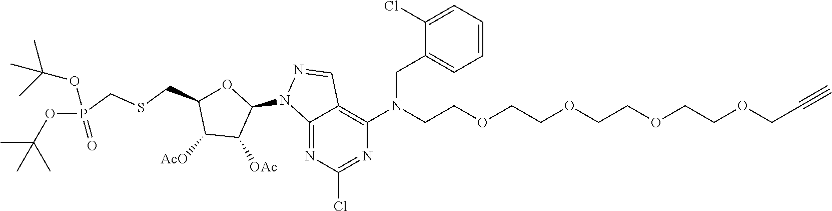 [SMILES: C#CCOCCOCCOCCOCCN(Cc1ccccc1Cl)c1nc(Cl)nc2c1cnn2[C@@H]1O[C@H](CSCP(=O)(OC(C)(C)C)OC(C)(C)C)[C@@H](OC(C)=O)[C@H]1OC(C)=O]

Product from step g (94.4 mg, 0.127 mmol) was dissolved in anhydrous THE (1 ml) and cooled in an ice-water bath. DIPEA (65 mg, 0.5 mmol) was added, followed by methanesulfonic acid, 1,1,1-trifluoro-, [bis(1,1-dimethylethoxy)phosphinyl]methyl ester (68.1 mg, 0.191 mmol). The resulting mixture was stirred at room temperature under nitrogen for 3 days, then carried to the subsequent step without purification. Ions found by LCMS: [M−2tBu]⁺=833.4, [(M−2tBu)/2]⁺=417.6.

### Step i

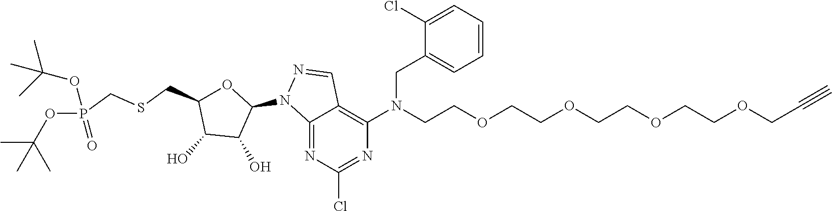 [SMILES: C#CCOCCOCCOCCOCCN(Cc1ccccc1Cl)c1nc(Cl)nc2c1cnn2[C@@H]1O[C@H](CSCP(=O)(OC(C)(C)C)OC(C)(C)C)[C@@H](O)[C@H]1O]

The reaction mixture from step h was diluted with MeOH (2 ml) and treated with K₂CO₃ (276.5 mg, 2 mmol). After stirring for 2 hours, the salt was filtered off and washed with acetonitrile. The filtrate was concentrated by rotary evaporation and purified through RPLC (100 g, 5 to 100% acetonitrile and water, using 0.1% TFA as modifier). Yield 45.7 mg, 41.7% over two steps. Ions found by LCMS: [M−tBu]⁺=806.2, [M−2tBu]⁺=750.6.

### Step j

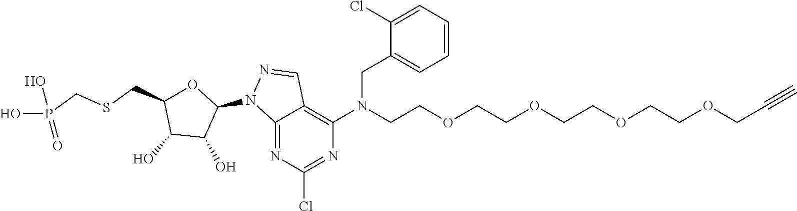 [SMILES: C#CCOCCOCCOCCOCCN(Cc1ccccc1Cl)c1nc(Cl)nc2c1cnn2[C@@H]1O[C@H](CSCP(=O)(O)O)[C@@H](O)[C@H]1O]

Product from step i (45.7 mg, 0.053 mmol) was dissolved in TFA (˜0.3 ml). The solution was stirred for 30 minutes then directly purified by HPLC (5 to 70% acetonitrile and water, using 0.1% TFA as modifier). Yield 37.5 mg, 94.3%. Ions found by LCMS: [M+Na]⁺=772.0, [M+H]⁺=750.0, [(M+2H)/2]⁺=375.7.

### Step k

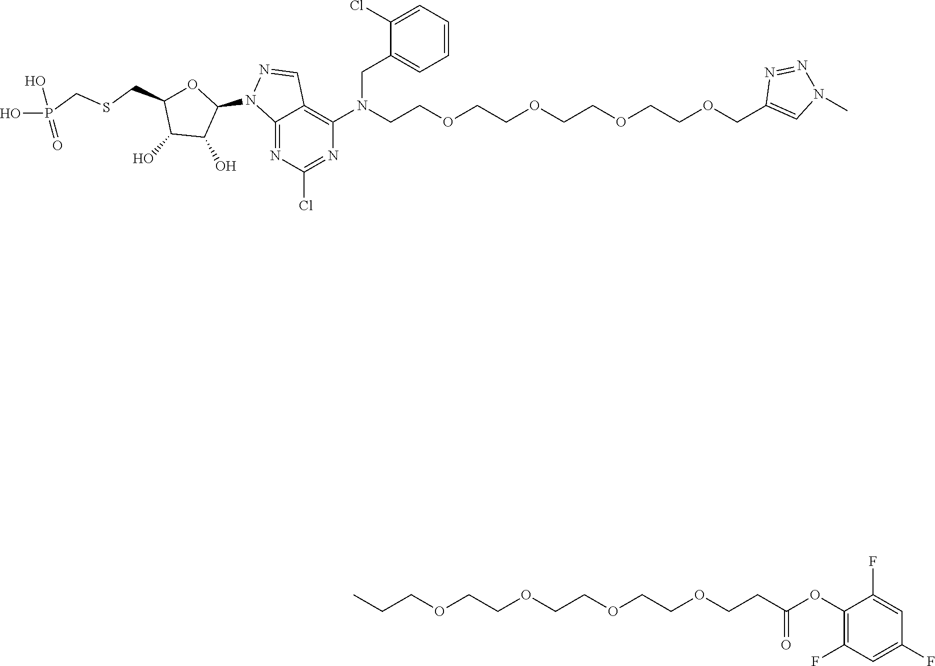

Product from step j (10 mg, 0.0133 mmol) was dissolved in MeOH (0.3 ml) and added with N₃-PEG4-trifluorophenol ester (6.8 mg, 0.016 mmol), DMF (0.4 ml), and a premixed mixture of THPTA (4.3 mg, 0.01 mmol), Cu₂SO₄ (1.6 mg, 0.01 mmol) and sodium ascorbate (19.8 mg, 0.1 mmol) in water (0.3 ml). The reaction was stirred for 30 minutes, then directly purified by HPLC (5 to 75% acetonitrile and water, using 0.1% TFA as modifier). Yield 11.5 mg, 73.8%. Ion found by LCMS: [(M+2H)/2]⁺=586.2.

### Synthesis of Conjugate 154

To a solution of SEQ ID NO: 80 (4.47 ml, 68 mg, 0.001227 mmol) in PBS 7.4 was added active ester described in Int-286 (11.5 mg, 0.009812 mmol) in DMF (0.4 ml). Addition DMF (0.4 ml×3) were used to wash the glassware and combined to the reaction mixture. The pH of the reaction mixture was slowly adjusted to ˜8.5 by 20× borate buffer solution (0.15 ml). After the reaction was gently rotated for 2 hours, a premixed solution (1 ml) of 150 mM histidine, 100 mM NH₄₀H and 70 mM HCl (pH 8.5) was then added, and the resulting mixture was gently rotated for 1.5 hours. It was purified by dialysis and SEC. Maldi TOF analysis of the purified final product gave an average mass of 61191 Da (DAR=5.8). Yield: 20.6 mg, 34.2%.

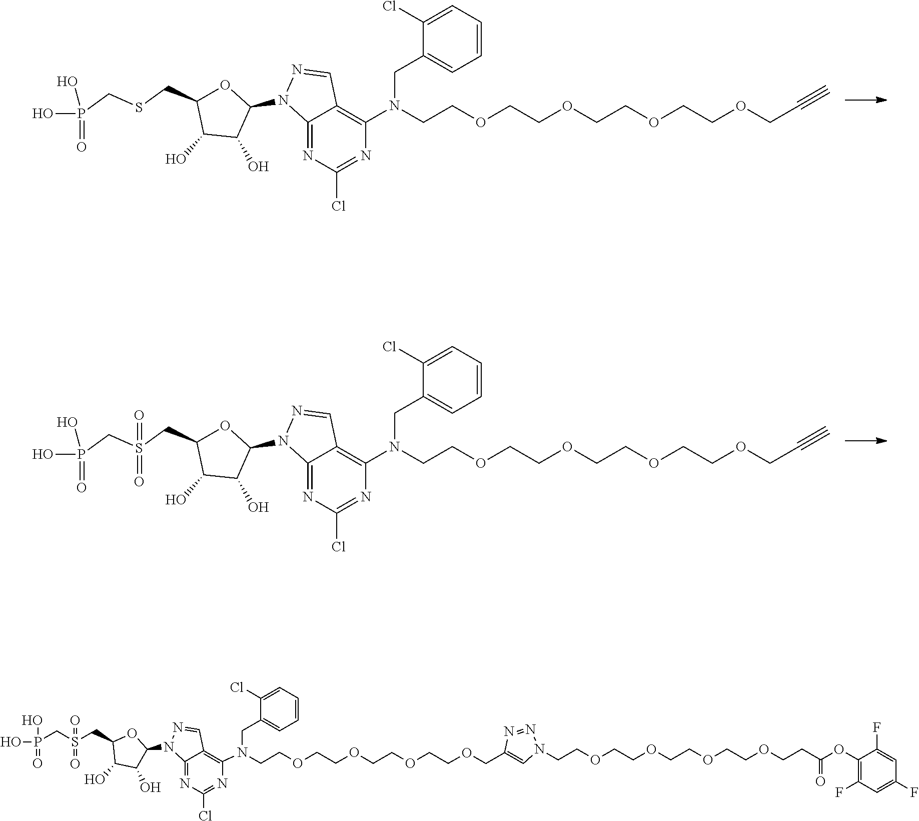 [SMILES: C#CCOCCOCCOCCOCCN(Cc1ccccc1Cl)c1nc(Cl)nc2c1cnn2[C@@H]1O[C@H](CSCP(=O)(O)O)[C@@H](O)[C@H]1O ; C#CCOCCOCCOCCOCCN(Cc1ccccc1Cl)c1nc(Cl)nc2c1cnn2[C@@H]1O[C@H](CS(=O)(=O)CP(=O)(O)O)[C@@H](O)[C@H]1O ; O=C(CCOCCOCCOCCOCCn1cc(COCCOCCOCCOCCN(Cc2ccccc2Cl)c2nc(Cl)nc3c2cnn3[C@@H]2O[C@H](CS(=O)(=O)CP(=O)(O)O)[C@@H](O)[C@H]2O)nn1)Oc1c(F)cc(F)cc1F]

### Synthesis of Int-290

### Step a

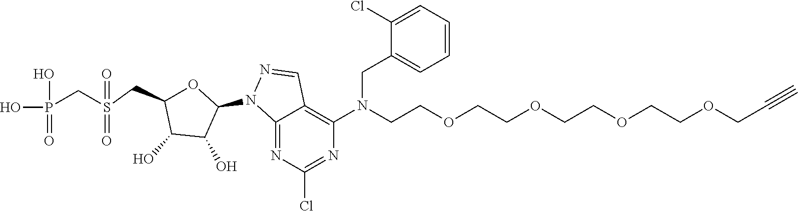 [SMILES: C#CCOCCOCCOCCOCCN(Cc1ccccc1Cl)c1nc(Cl)nc2c1cnn2[C@@H]1O[C@H](CS(=O)(=O)CP(=O)(O)O)[C@@H](O)[C@H]1O]

To a solution of product from step g as described in Int-286 (13 mg, 0.0173 mmol) in a mixture of DMF (0.2 ml) and MCOH (0.4 ml) was added Oxone (43 mg, 0.07 mmol) and water (0.2 ml). The resulting mixture was stirred overnight, then the solid was filtered off and washed with MeOH. The filtrate was concentrated and directly purified by HPLC (5 to 75% acetonitrile and water, using 0.1% TEA as modifier). Yield 12.3 mg, 90.8%. Ion found by LCM: [M+2H/]=782.0.

### Step b

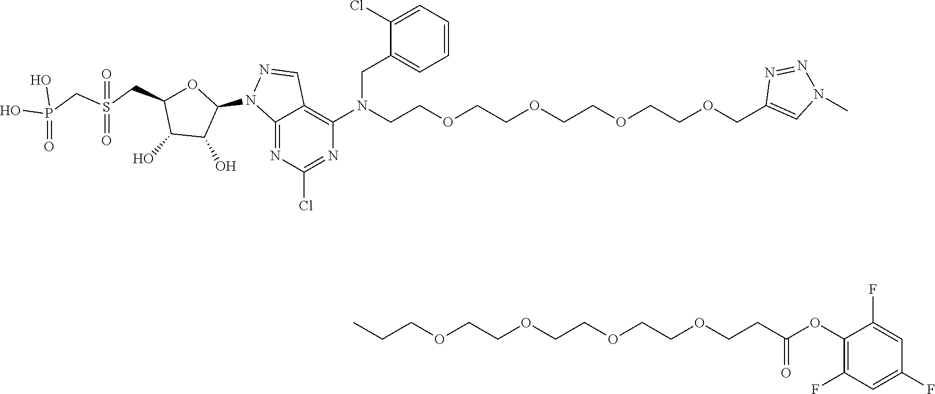

Product from step a (10 mg, 0.0128 mmol) was dissolved in MeOH (0.2 ml) and added with N₃-PEG4-Trifluorophenol ester (6.5 mg, 0.0153 mmol), DMF (0.4 ml) and a premixed mixture of THPTA (4.3 mg, 0.01 mmol), Cu₂SO₄ (1.6 mg, 0.1 mmol) and sodium ascorbate (19.8 mg, 0.1 mmol) in water (0.3 ml). The reaction was stirred for 1 hour, then directly purified by HPLC (5 to 75% acetonitrile and water, using 0.1% TEA as modifier). Yield 8.6 mg, 55.8%. Ion found by LCMS: [(M+2H)/2]+=602.2.

### Synthesis of Conjugate 157

The title compound was prepared analogously to Conjugate 151, where Int-286 was replaced with Int-290. Maldi TOF analysis of the purified final product gave an average mass of 62,430 Da (DAR=6.8). Yield: 26.6 mg, 53.3%.

### Synthesis of Int-291

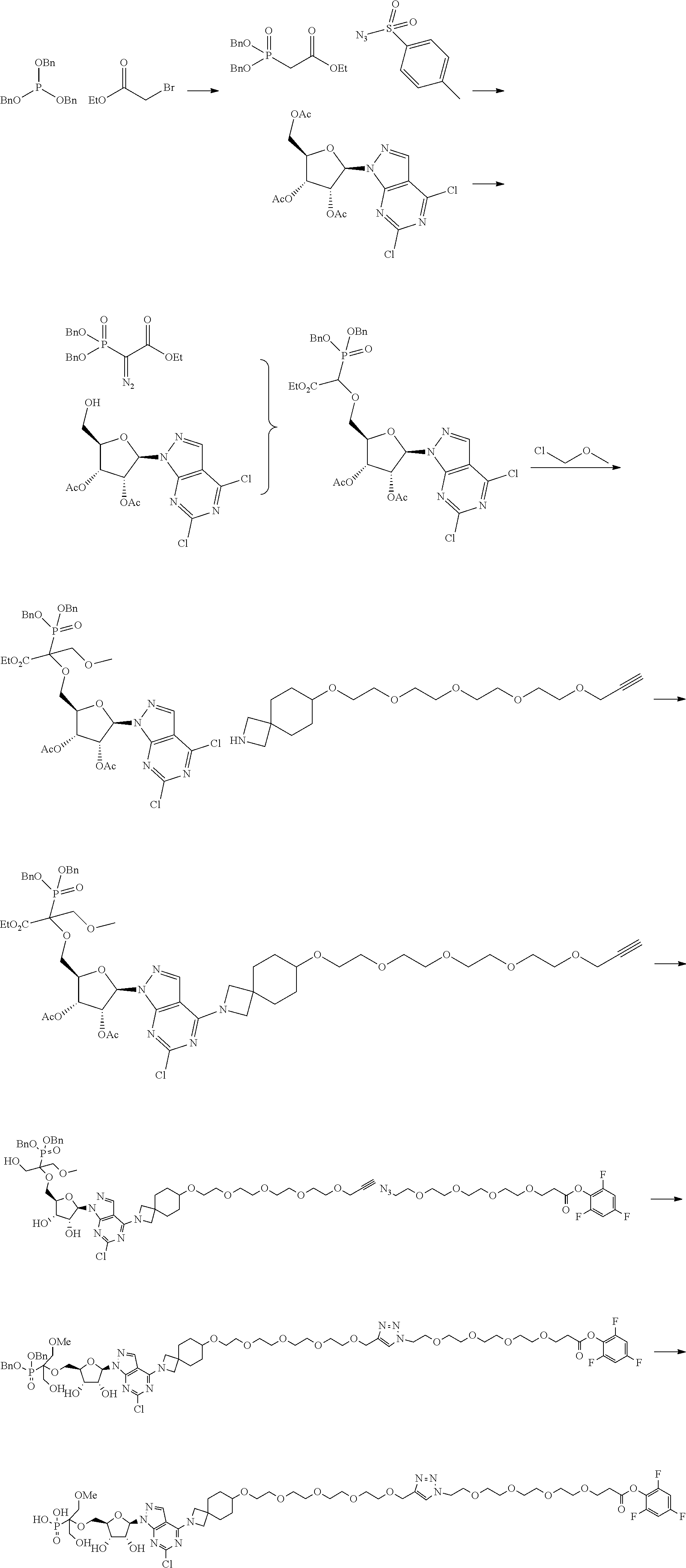

### Step a

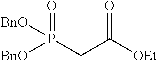 [SMILES: CCOC(=O)CP(=O)(OCc1ccccc1)OCc1ccccc1]

Bromo ethyl acetate (11.3 g, 67.9 mmol) was added to tribenzyl phosphite (10 g, 27.1 mmol) and the mixture was stirred for 4 hours at 10° C. The mixture was cooled to room temperature and purified by silica gel chromatography (ethyl acetate/hexanes) to afford the product as a clear viscous oil. Yield 6.3 g, 66%. Ion(s) found by LCMS: [M+H]+=349.2.

### Step b

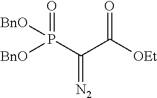

Tosyl azide (5.7 g, 28.7 mmol, in 50 mL DCM) was added dropwise, via an addition funnel over a 2 hour period, to a cooled (000 ice bath) mixture of sodium t-butoxide and dibenzyl-ethyl-phosphono acetate (10 g, 28.7 mmol in 100 mL) of DCM. The mixture was stirred overnight allowing the ice bath to gradually rise to ambient temperature. The mixture was filtered through celite and concentrated. The crude residue was purified by silica gel chromatography (hexanes/ethyl acetate). Yield 7.2 g, 67%. Ion(s) found by LCMS: [M+H]⁺=375.2.

### Step c

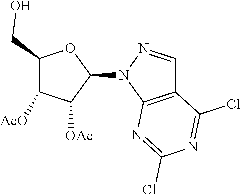 [SMILES: CC(=O)O[C@@H]1[C@H](OC(C)=O)[C@@H](CO)O[C@H]1n1ncc2c(Cl)nc(Cl)nc21]

The triacetate intermediate B (7 g, 15.7 mmol) was dissolved in MTBE (100 mL), then mixed with lipase Candida rugosa (4.5 g) followed by 200 mL of phosphate buffer 0.2 M (pH 7.4). The reaction was stirred at 40° C. for 16 hours at which time the mixture was filtered through a plug of celite. The mixture was extracted into ethyl acetate and the combined organics were dried over sodium sulfate, concentrated, and purified over silica gel chromatography (Ethyl acetate/hexanes). Yield 5.5 g, 86%. Ion(s) found by LCMS: [M+H]⁺=405.2.

### Step d

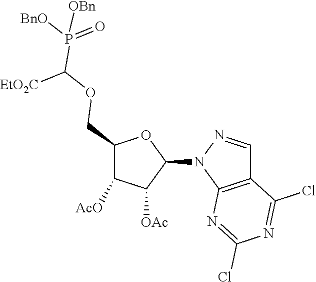 [SMILES: CCOC(=O)C(OC[C@H]1O[C@@H](n2ncc3c(Cl)nc(Cl)nc32)[C@H](OC(C)=O)[C@@H]1OC(C)=O)P(=O)(OCc1ccccc1)OCc1ccccc1]

The alcohol described in the previous step (2.6 g, 6.4 mmol) was dissolved in toluene (100 mL) in a sealed tube. The diazo intermediate described in step b of this example (4.8 g, 12.8 mmol) was added and the mixture was evacuated by vacuum flushed with nitrogen (3×). Rhodium tetra acetate (200 mg) was added and the mixture was again evacuated and flushed with nitrogen (3×). The tube was sealed and placed in an oil bath and stirred at 110° C. for 16 hours. The mixture was cooled and filtered through a plug of celite. The solvent was removed by the rotary evaporator and the crude residue was purified by silica gel chromatography (hexanes/ethyl acetate). Yield 2.7 g, 56%. Ion(s) found by LCMS: [M+H]⁺=751.2.

### Step e

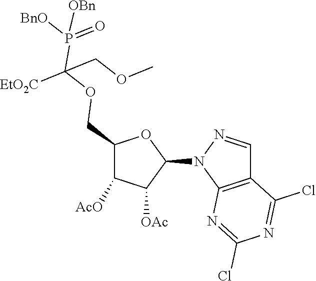 [SMILES: CCOC(=O)C(COC)(OC[C@H]1O[C@@H](n2ncc3c(Cl)nc(Cl)nc32)[C@H](OC(C)=O)[C@@H]1OC(C)=O)P(=O)(OCc1ccccc1)OCc1ccccc1]

The ester described in the previous step of this example (500 mg, 0.67 mmol) was dissolved in THE and cooled to −15° C. under an atmosphere of nitrogen. Sodium HMDS (0.73 mL, 0.73 mmol, 1M in THF) was added and the mixture was stirred for 10 minutes at −15° C. TBAI (245 mg, 0.67 mmol) was added followed by MOM chloride (120 mg, 1.33 mmol) and the reaction was stirred for 20 minutes. The reaction was quenched at −15° C. with saturated aqueous ammonium chloride, extracted into ethyl acetate (3×), and the combined organic extracts were dried over sodium sulfate and the solvent removed on the rotary evaporator. The crude residue was purified by silica gel chromatography (hexanes/ethyl acetate to afford the product as a clear oil. Yield 323 mg, 61%. Ion(s) found by LCMS: [M+H]⁺=795.2.

### Step f

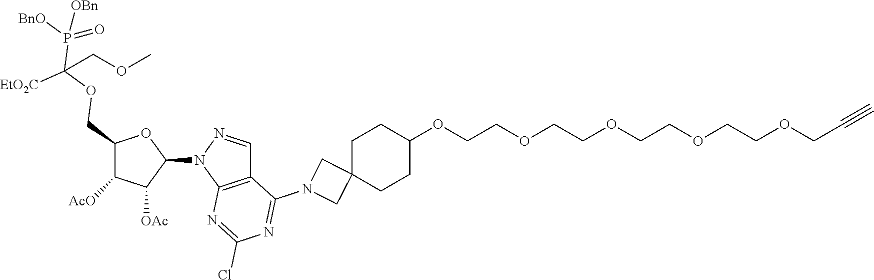 [SMILES: C#CCOCCOCCOCCOCCOC1CCC2(CC1)CN(c1nc(Cl)nc3c1cnn3[C@@H]1O[C@H](COC(COC)(C(=O)OCC)P(=O)(OCc3ccccc3)OCc3ccccc3)[C@@H](OC(C)=O)[C@H]1OC(C)=O)C2]

A mixture of product from previous step (110 mg, 0.138 mmol), amine (73 mg, 0.207 mmol) (described in step a synthesis of Int-259), and DIEA (0.07 mL, 0.414 mmol) in MeOH (5 mL) was heated at 50° C. for 1 hour. After complete consumption of the starting materials, the reaction mixture was cooled to room temperature and volatiles were removed by rotatory evaporation. The crude residue adsorbed on silica gel and purified by flash column chromatography (DCM: 10% MeOH in DCM) to afford the desired product as a white foam. Yield 120 mg, 78%. Ion(s) found by LCMS: [M+H]⁺=1114.2.

### Step g

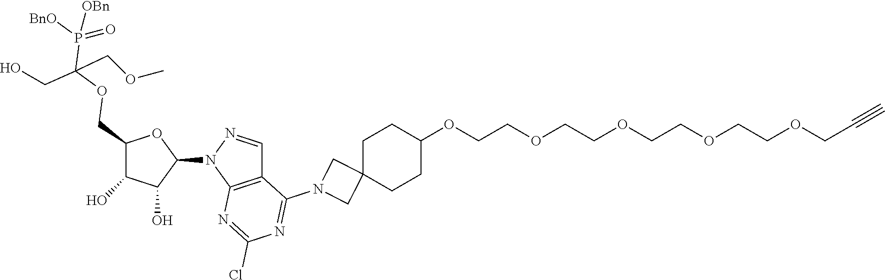 [SMILES: C#CCOCCOCCOCCOCCOC1CCC2(CC1)CN(c1nc(Cl)nc3c1cnn3[C@@H]1O[C@H](COC(CO)(COC)P(=O)(OCc3ccccc3)OCc3ccccc3)[C@@H](O)[C@H]1O)C2]

To a solution of compound from previous step (120 mg, 0.107 mmol) in ethanol (5 mL) that had been cooled to 0° C. using an ice bath, was added calcium dichloride (36 mg, 0.323 mmol) followed by sodium borohydride (12.2 mg, 0.323 mmol). The mixture was warmed to ambient temperature, where it was stirred for an additional 1 hour, before being placed back into an ice bath. Once cooled, the reaction was quenched with 1N HCl (3 mL), and then diluted with EtOAc (30 mL). The flask was removed from the bath and allowed to stir at room temperature for 20 minutes. The organic layer was washed with additional 1 N HCl and the combined aqueous layers were extracted with EtOAc (2×10 mL). The combined organic layers were then washed with brine, dried over sodium sulfate, filtered, and concentrated under reduced pressure. The resulting crude residue was purified by RPLC (10% to 100% ACN/water, 0.1% TFA modifier) to yield the title compound as white solid. Yield 41 mg, 39%. Ion(s) found by LCMS: [M+H]⁺=988.4.

### Step h

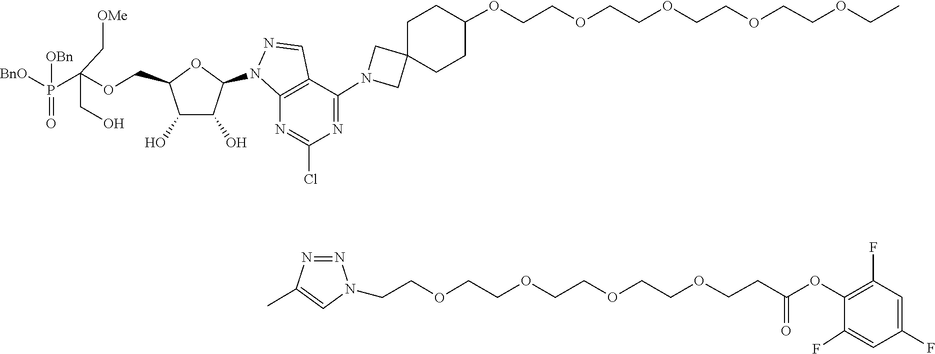

To a solution of product from the previous step (41 mg, 0.041 mmol) and intermediate A (17 mg, 0.041 mmol). dissolved in DMF:H₂O (1:3, 1.5 mL) were cooled to 0° C. To this a pre-mixed solution of a solution of copper(II) sulfate (1 mg, 0.005 mmol), sodium ascorbate (32 mg, 0.163 mmol), and BTTA (5 mg, 0.011 mmol) dissolved in water (0.5 mL) was added and stirred for 5 minutes at the same temperature and gradually warmed to room temperature and stirred for an additional 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 32 mg, 54%. Ion(s) found by LCMS [(M+2H)/2]⁺=705.4.

### Step i

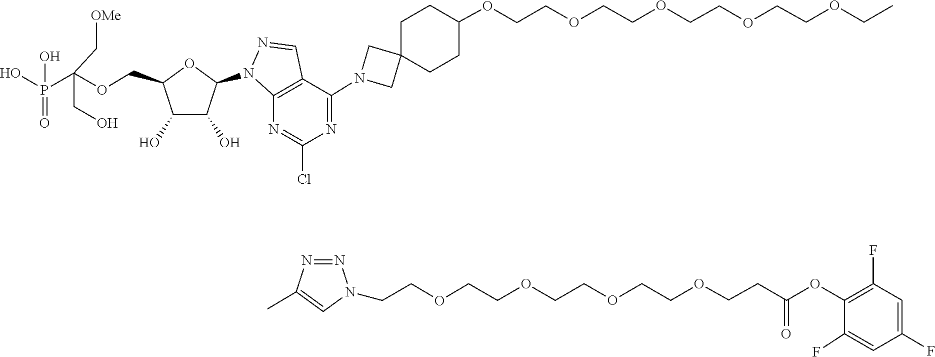

To a solution of compound from previous step (30 mg, 0.0213 mmol) in MeOH (3 mL) at ambient temperature was added 5% Pd/C (18 mg). The resulting mixture was stirred at room temperature under an atm of hydrogen for 30 minutes. Reaction mixture was filtered through a short pad of celite. Concentration of the filtrate followed by purification by RPLC (ACN: H₂O, 0.1% TFA modifier) yielded the title compound as a white solid. Yield 19 mg, 72%. Ion(s) found by LCMS [(M+2H)/2]⁺=615.5.

### Synthesis of Conjugate 158

Int-291 (8.9 mg, 0.00724 mmol) was added to SEQ ID NO: 80 (50 mg, 5.2 mL in PBS at pH 8.4) as described in the general conjugation procedure. Maldi TOF analysis of the purified final product gave an average mass of 59,745 Da. (DAR=4.2). Yield 26 mg, 51%.

### Synthesis of Int-296

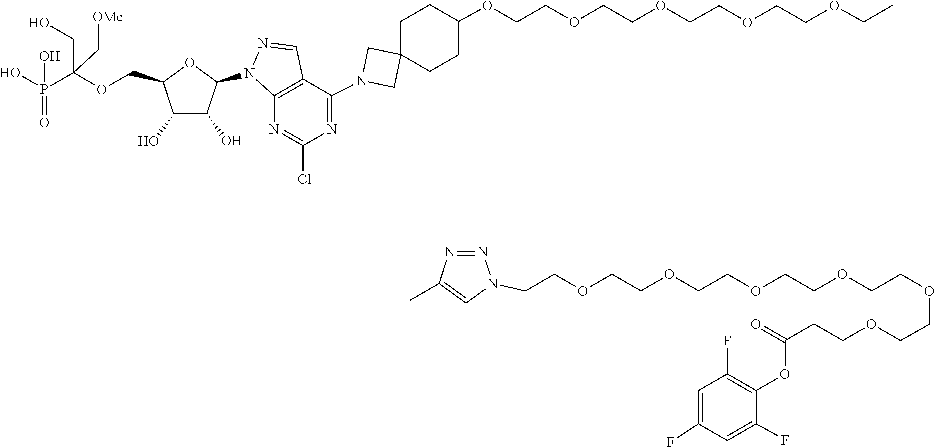

The title compound was prepared analogously to Int-291 where intermediate A was replaced with azidoPEG₆TFP ester prepared as described in step a of Synthesis of Int-281). The product was a white solid. Ion(s) found by LCMS: [(M+2H)/2]⁺=659.4.

### Synthesis of Conjugate 163

Int-296 (21.7 mg, 0.0217 mmol) was added to SEQ ID NO: 80 (150 mg, 11.63 mL in PBS at pH 8.4) as described in the general conjugation procedure. Maldi TOF analysis of the purified final product gave an average mass of 61,914 Da. (DAR=5.7). Yield 101 mg, 67%.

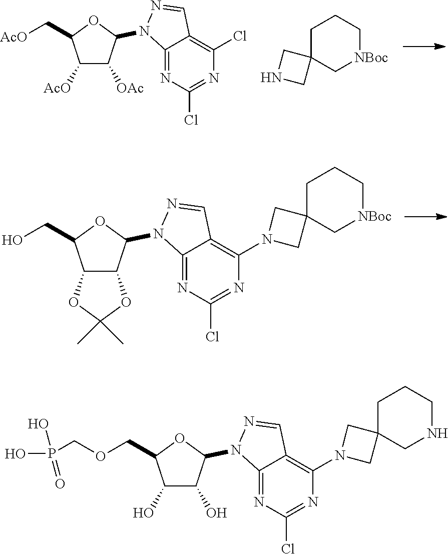 [SMILES: CC(=O)OC[C@H]1O[C@@H](n2ncc3c(Cl)nc(Cl)nc32)[C@H](OC(C)=O)[C@@H]1OC(C)=O ; CC(C)(C)OC(=O)N1CCCC2(CNC2)C1 ; CC(C)(C)OC(=O)N1CCCC2(C1)CN(c1nc(Cl)nc3c1cnn3[C@@H]1O[C@H](CO)[C@H]3OC(C)(C)O[C@H]31)C2 ; O=P(O)(O)COC[C@H]1O[C@@H](n2ncc3c(N4CC5(CCCNC5)C4)nc(Cl)nc32)[C@H](O)[C@@H]1O]

### Synthesis of Int-287

### Step a

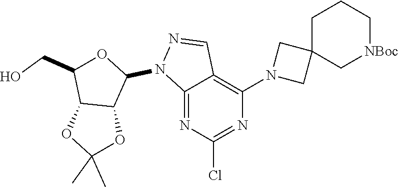

A solution of Intermediate B (11 g, 22 mmol, Example ??intermediate B), 2,6-diazaspiro (5 g, 22 mmol) and triethylamine (3.3 mL, 24 mmol) in anhydrous EtOH (22 mL) was stirred at room temperature for 30 min. The solution was concentrated, and the residue was dissolved in 7 M NH3/MeOH (50 mL) and the mixture was stirred overnight. The reaction mixture was concentrated to dryness under reduced pressure, then dissolved in acetone (32 ml). 2,2-dimethoxypropane (32.48 mL, 265 mmol) and p-TsOH (5.1 g, 26 mmol) was added, and the mixture was stirred overnight at room temperature. The reaction mixture was concentrated to dryness under reduced pressure and purified by column chromatography (DCM: 10% MeOH in DCM) to yield product. The product was a white foam. Yield 9.9 g, 81%. Ion(s) found by LCMS: [M+H]⁺=551.2.

### Step b

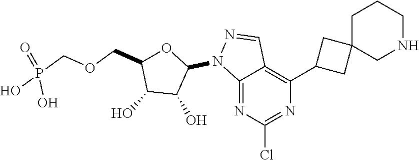

A solution of acetonide derivative from previous step (6.2 g, 11 mmol) and [bis(tert-butoxy)carbonyl]methyl (trifluoromethyl)sulfonate (12 g, 33 mmol) in dry DMF was cooled using ice-water bath. 60% NaH in mineral oil (2.2 g, 56 mmol) was added to this and stirred at room temperature for 2 h. The reaction mixture was quenched with saturated ammonium chloride and extracted with EtOAc. The organic phase was washed with brine and dried over sodium sulfate concentrated to give crude product which was used in the next step without purification. Crude product was dissolved in 50% aqueous TFA (50 mL) and stirred at room temperature for 12 h, concentrated and the residue was purified by semi-preparative HPLC (10-100% ACN: H₂O, 0.1% TFA modifier). The product was a white solid. Yield 3.9 g, 68%. Ion(s) found by LCMS: [M+H]⁺=505.1.

### Synthesis of Int-292

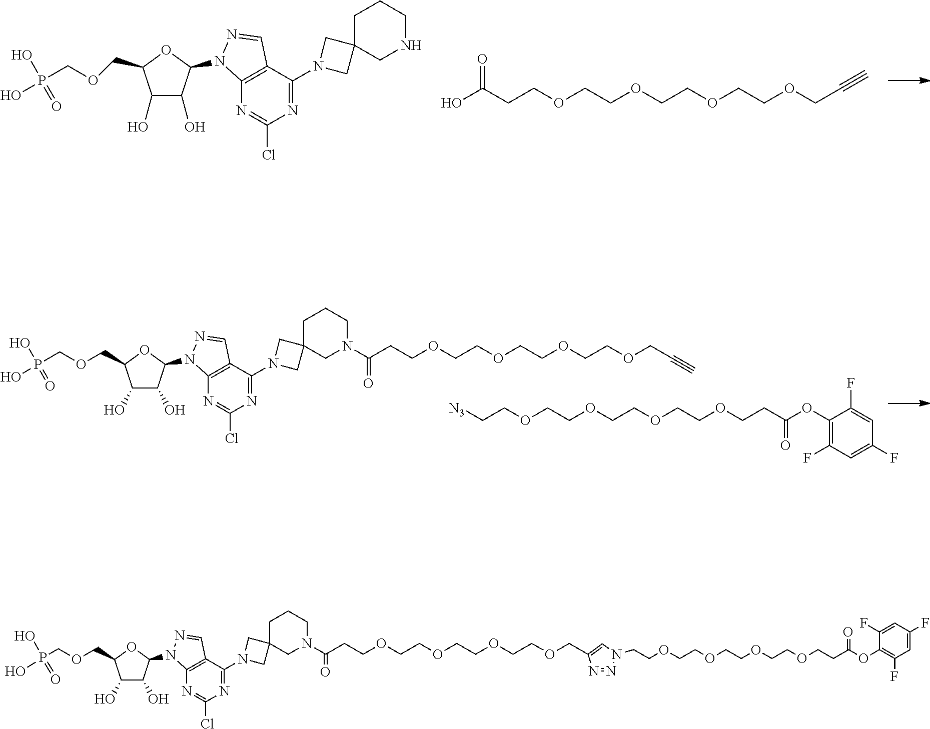 [SMILES: O=P(O)(O)COC[C@H]1O[C@@H](n2ncc3c(N4CC5(CCCNC5)C4)nc(Cl)nc32)C(O)C1O ; C#CCOCCOCCOCCOCCC(=O)O ; C#CCOCCOCCOCCOCCC(=O)N1CCCC2(C1)CN(c1nc(Cl)nc3c1cnn3[C@@H]1O[C@H](COCP(=O)(O)O)[C@@H](O)[C@H]1O)C2 ; [N-]=[N+]=NCCOCCOCCOCCOCCC(=O)Oc1c(F)cc(F)cc1F ; O=C(CCOCCOCCOCCOCCn1cc(COCCOCCOCCOCCC(=O)N2CCCC3(C2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

### Step a

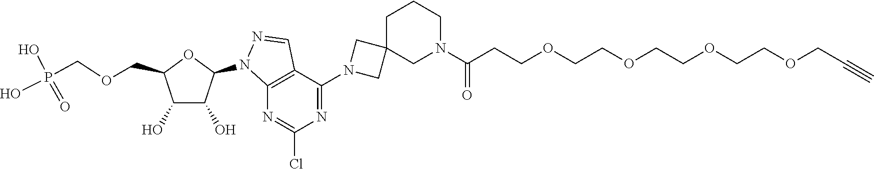 [SMILES: C#CCOCCOCCOCCOCCC(=O)N1CCCC2(C1)CN(c1nc(Cl)nc3c1cnn3[C@@H]1O[C@H](COCP(=O)(O)O)[C@@H](O)[C@H]1O)C2]

To a solution of Int-287 (30 mg, 0.059 mmol) and propargyl-PEG4-carboxylic acid (15 mg, 0.059 mmol) in DMF (1 ml) was added DIEA (0.062 mL, 0.35 mmol) and HATU (34 mg, 0.09 mmol). The reaction solution was stirred at room temperature for 2 hours. The reaction was concentrated and the residue was purified by semi-preparative HPLC (5% to 100% ACN/water, 0.1% TFA modifier) to give the product as an off-white foam. Yield 28 mg, 64%. Ion(s) found by LCMS: [M+H]⁺=747.2.

### Step b

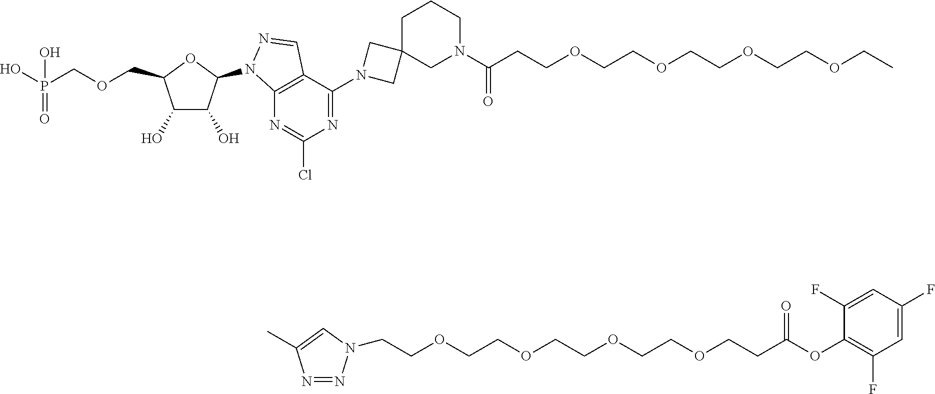

To a solution of the product from the previous step (28 mg, 0.037 mmol) and azido-PEG4-trifluorophenyl ester (20 mg, 0.049 mmol) in DMF (0.3 mL) was added a pre-mixed solution of a solution of copper(II) sulfate (1.5 mg, 0.001 mmol), sodium ascorbate (7.4 mg, 0.037 mmol), and THPTA (8.1 mg, 0.018 mmol) in water (0.3 mL). The solution stirred for 15 minutes. When the reaction was complete (by HPLC), the product was purified by reversed phase HPLC (10% to 100% ACN/water, 0.1% TFA modifier). The product was a sticky oil. Yield 33 mg, 75%. Ion(s) found by LCMS [(M+2H)/2]⁺=584.6.

### Synthesis of Conjugate 159

Int-292 (6.4 mg, 0.005 mmol) in DMF (0.5 mL) was added to SEQ ID NO: 80 (50 mg, 3.8 mL, 0.000905) in PBS at pH 7.4 as described in the conjugation procedure. Maldi TOF analysis of the purified final product gave an average mass of 59,218 (DAR=3.9). Yield 26.9 mg, 53.7%.

### Synthesis of Int-293

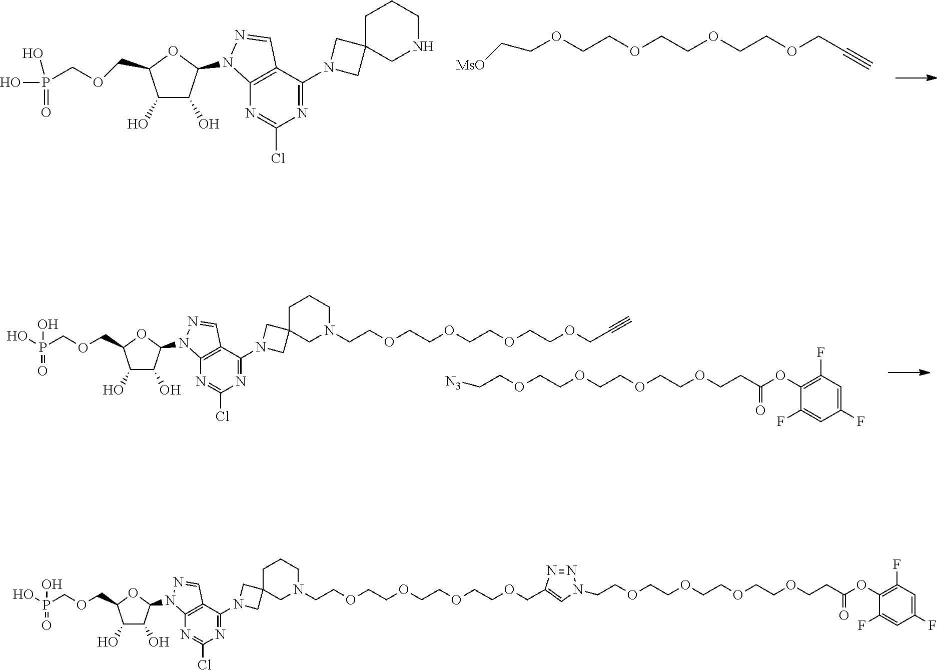 [SMILES: O=P(O)(O)COC[C@H]1O[C@@H](n2ncc3c(N4CC5(CCCNC5)C4)nc(Cl)nc32)[C@H](O)[C@@H]1O ; C#CCOCCOCCOCCOCCOS(C)(=O)=O ; C#CCOCCOCCOCCOCCN1CCCC2(C1)CN(c1nc(Cl)nc3c1cnn3[C@@H]1O[C@H](COCP(=O)(O)O)[C@@H](O)[C@H]1O)C2 ; [N-]=[N+]=NCCOCCOCCOCCOCCC(=O)Oc1c(F)cc(F)cc1F ; O=C(CCOCCOCCOCCOCCn1cc(COCCOCCOCCOCCN2CCCC3(C2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

### Step a

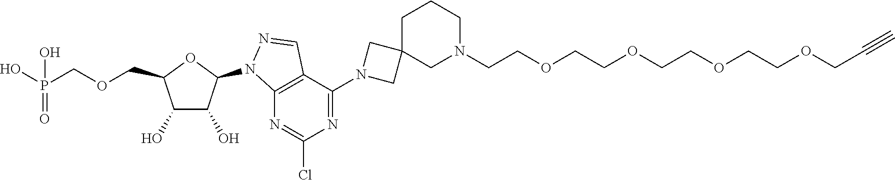 [SMILES: C#CCOCCOCCOCCOCCN1CCCC2(C1)CN(c1nc(Cl)nc3c1cnn3[C@@H]1O[C@H](COCP(=O)(O)O)[C@@H](O)[C@H]1O)C2]

To a solution of Int-287 (50 mg, 0.1 mmol) and propargyl-PEG4-mesyl ester (63 mg, 0.2 mmol) in acetonitrile (2 mL) was added Et₃N (0.082 ml, 0.6 mmol). The reaction solution stirred at 70° C. for 30 min and then concentrated. The residue was purified by semi-preparative HPLC (5% to 95% acetonitrile and water, using 0.1% TFA as modifier). Yield 52 mg, 73%. LCMS: [M+H]⁺=719.2.

### Step b

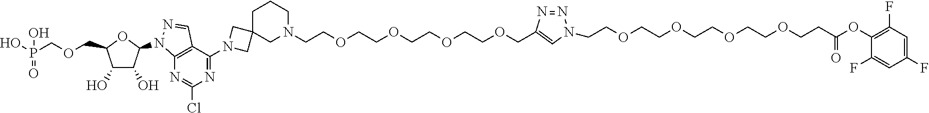 [SMILES: O=C(CCOCCOCCOCCOCCn1cc(COCCOCCOCCOCCN2CCCC3(C2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

To a solution of the product from the previous step (40 mg, 0.055 mmol) and azido-PEG4-trifluorophenyl ester described in Int-291 (24 mg, 0.057 mmol) in DMF (0.5 mL) was added a pre-mixed solution of a solution of copper(II) sulfate (0.002 mg, 0.013 mmol), sodium ascorbate (11 mg, 0.055 mmol), and THPTA (12 mg, 0.027 mmol) in water (0.5 mL). The reaction solution stirred for 15 minutes.

When the reaction was complete (by HPLC), the product was purified by reversed phase HPLC (10% to 100% ACN/water, 0.1% TFA modifier). The product was a white solid. Yield 42 mg, 66%. Ion(s) found by LCMS [(M+2H)/2]⁺=570.7.

### Synthesis of Conjugate 160

Int-293 (6.2 mg, 0.005 mmol) in DMF (0.4 mL) was added to SEQ ID NO: 80 (50 mg in 3.8 mL PBS 7.4, 0.000905 mmol) in PBS at pH 7.4 as described in the general conjugation procedure. Maldi TOF analysis of the purified final product gave an average mass of 58,323 Da. (DAR=3.1). Yield 27.7 mg, 55.4%.

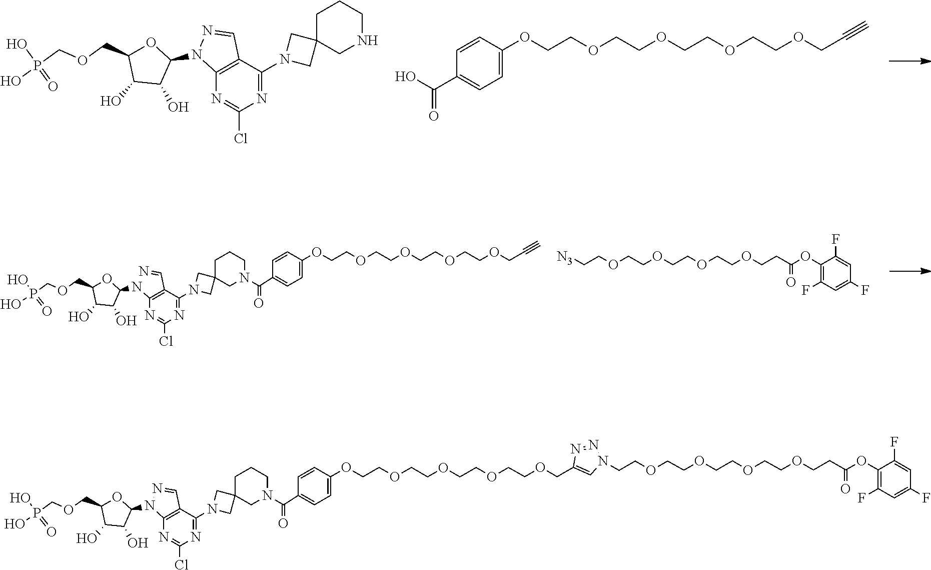 [SMILES: O=P(O)(O)COC[C@H]1O[C@@H](n2ncc3c(N4CC5(CCCNC5)C4)nc(Cl)nc32)[C@H](O)[C@@H]1O ; C#CCOCCOCCOCCOCCOc1ccc(C(=O)O)cc1 ; C#CCOCCOCCOCCOCCOc1ccc(C(=O)N2CCCC3(C2)CN(c2nc(Cl)nc4c2cnn4[C@@H]2O[C@H](COCP(=O)(O)O)[C@@H](O)[C@H]2O)C3)cc1 ; [N-]=[N+]=NCCOCCOCCOCCOCCC(=O)Oc1c(F)cc(F)cc1F ; O=C(CCOCCOCCOCCOCCn1cc(COCCOCCOCCOCCOc2ccc(C(=O)N3CCCC4(C3)CN(c3nc(Cl)nc5c3cnn5[C@@H]3O[C@H](COCP(=O)(O)O)[C@@H](O)[C@H]3O)C4)cc2)nn1)Oc1c(F)cc(F)cc1F]

### Synthesis of Int-302

### Step a

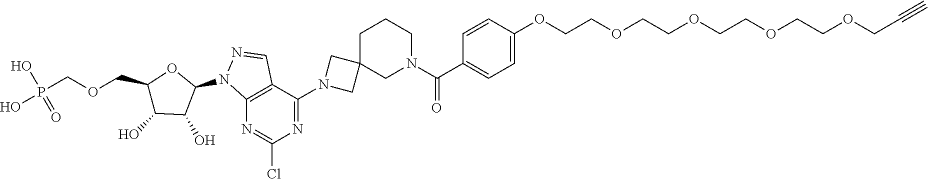 [SMILES: C#CCOCCOCCOCCOCCOc1ccc(C(=O)N2CCCC3(C2)CN(c2nc(Cl)nc4c2cnn4[C@@H]2O[C@H](COCP(=O)(O)O)[C@@H](O)[C@H]2O)C3)cc1]

To a solution of Int-287 (50 mg, 0.1 mmol) and PEG4-benzoic acid (35 mg, 0.1 mmol) in DMF (1 ml) was added DIEA (0.1 ml, 0.6 mmol) and HATU (114 mg, 0.3 mmol). The reaction solution was stirred at room temperature for 2 hours. The reaction was concentrated and the residue was purified by semi-preparative HPLC (5% to 100% ACN/water, 0.1% TFA modifier) to give the product as an off-white foam. Yield 54 mg, 64%. Ion(s) found by LCMS: [M+H]⁺=839.2.

### Step b

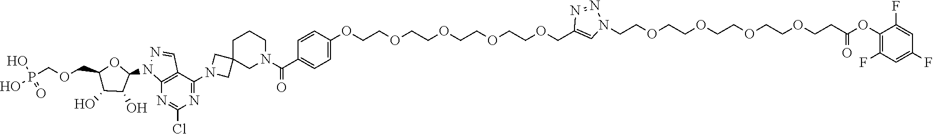 [SMILES: O=C(CCOCCOCCOCCOCCn1cc(COCCOCCOCCOCCOc2ccc(C(=O)N3CCCC4(C3)CN(c3nc(Cl)nc5c3cnn5[C@@H]3O[C@H](COCP(=O)(O)O)[C@@H](O)[C@H]3O)C4)cc2)nn1)Oc1c(F)cc(F)cc1F]

To a solution of the product from the previous step (34 mg, 0.04 mmol) and azido-PEG4-trifluorophenyl ester (22 mg, 0.05 mmol) in DMF (0.4 mL) was cooled to 0° C., and treated with a pre-mixed solution of a solution of copper(II) sulfate (1.6 mg, 0.01 mmol), sodium ascorbate (8.0 mg, 0.04 mmol), and THPTA (8.8 mg, 0.02 mmol) dissolved in water (0.4 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a white solid. Yield 36 mg, 70%. Ion(s) found by LCMS [(M+2H)/2]⁺=630.7.

### Synthesis of Conjugate 169

Int-302 (9.1 mg, 0.007 mmol) in DMF (0.3 mL) was added to SEQ ID NO: 80 (50 mg in 3.8 mL PBS 7.4, 0.0009 mmol) as described in the general conjugation procedure. Maldi TOF analysis of the purified final product gave an average mass of 60,782 Da. (DAR=5). Yield 25.3 mg, 50.6%.

### Synthesis of Int-305

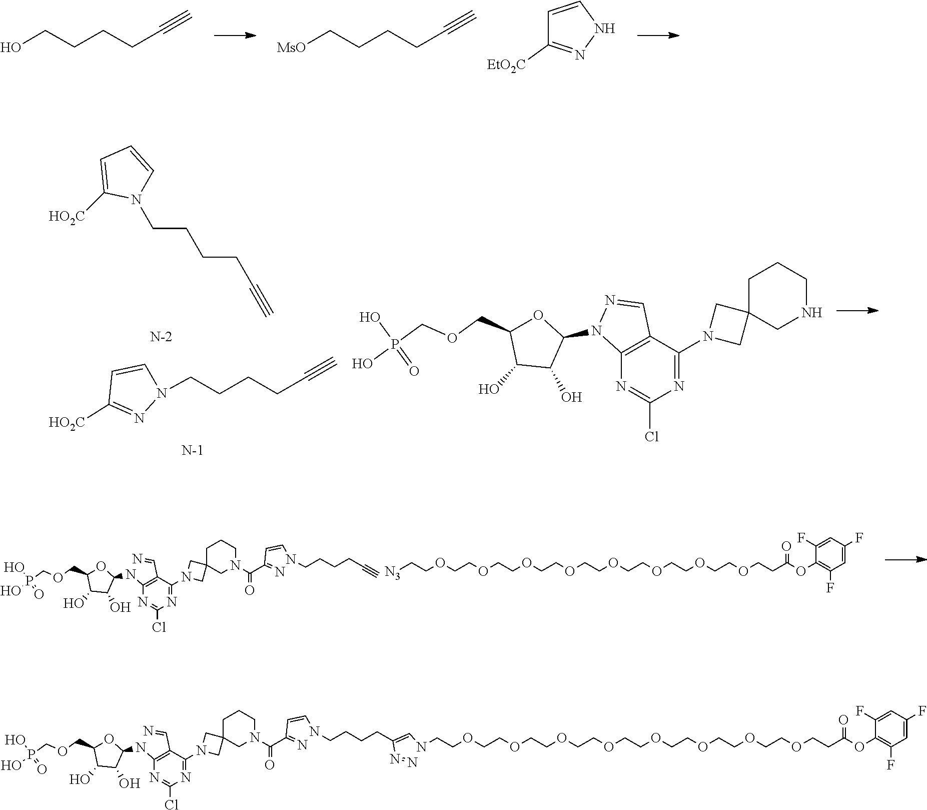

### Step a

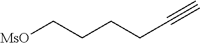

To a solution of alcohol (4.9 g, 98 mmol) in DCM (100 ml) was added Et₃N (14 ml, 100 mmol) and methanesulfonyl chloride (4.2 ml, 55 mmol). The solution was stirred at room temperature for 2 hours, washed with HCl (0.5N, aq), dis H₂O and brine. The organic layer was separated and dried over sodium sulfate, filtered, and concentrated to yield product (5-Hexyn-1-yl mesylate). The product was a white solid. Yield 7.1 g, 80%. Ion(s) found by LCMS: [M+H]⁺=177.0.

### Step b

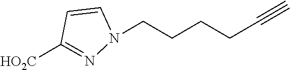

To a solution of ethyl 2H-pyrazole-3-carboxylate (1.4 g, 10 mmol) and step-a product (2.6 g, 15 mmol) in DMF (10 ml) was added K₂CO₃ (1.3 g, 10 mmol). The reaction mixture was heated at 70° C. for 1.5 days. After cooling to room temperature, the salt was filtered off and washed with acetonitrile. The filtrate was concentrated and the residue was dissolved in MeOH (5 ml) and treated with a solution of LiOH monohydrate (0.47 g, 20 mmol) in water (5 ml). The mixture was stirred at room temperature for 2 hours, then acidified with TFA and residue was purified by semi-preparative HPLC (5% to 100% ACN/water, 0.1% TFA modifier) to give N-1 acid as a solid. The N-1 regioisomer was assigned by a ¹H NMR showing pyrazole aromatic protons at 6.79 ppm and 7.73 ppm respectively, which is consistent to a commercially available 1-methylpyrazole-3-carboxylic acid. Yield 0.6 g, 32%. Ion(s) found by LCMS: [M+H]⁺=193.0.

### Step c

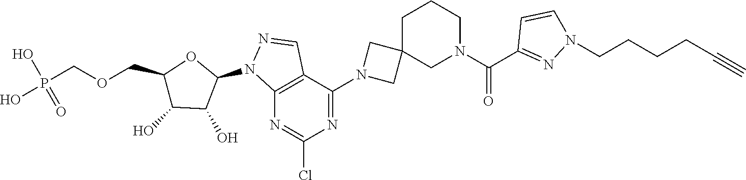 [SMILES: C#CCCCCn1ccc(C(=O)N2CCCC3(C2)CN(c2nc(Cl)nc4c2cnn4[C@@H]2O[C@H](COCP(=O)(O)O)[C@@H](O)[C@H]2O)C3)n1]

To a solution of Int-287 (50 mg, 0.1 mmol) and pyrazole-acid (138 mg, 0.7 mmol) in DMF was added DIEA (0.37 mL, 2.1 mmol) and HATU (410 mg, 1.1 mmol). The reaction solution was stirred at room temperature for 2 hours. The reaction was concentrated and the residue was purified by semi-preparative HPLC (5% to 100% ACN/water, 0.1% TFA modifier) to give the product as an off-white foam. Yield 270 mg, 55%. Ion(s) found by LCMS: [M+H]⁺=679.2.

### Step d

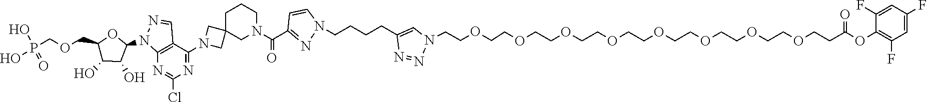 [SMILES: O=C(CCOCCOCCOCCOCCOCCOCCOCCOCCn1cc(CCCCn2ccc(C(=O)N3CCCC4(C3)CN(c3nc(Cl)nc5c3cnn5[C@@H]3O[C@H](COCP(=O)(O)O)[C@@H](O)[C@H]3O)C4)n2)nn1)Oc1c(F)cc(F)cc1F]

To a solution of the product from the previous step (50 mg, 0.073 mmol) and azido-PEG8-trifluorophenyl ester (prepared as described in step a of Synthesis of Int-282) (52 mg, 0.08 mmol) in DMF (0.4 mL) was cooled to 0° C., and treated with a pre-mixed solution of a solution of copper(II) sulfate (3 mg, 0.02 mmol), sodium ascorbate (43 mg, 0.22 mmol), and THPTA (16 mg, 0.036 mmol) dissolved in water (0.4 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a colorless oil. Yield 61 mg, 64%. Ion(s) found by LCMS [(M+2H)/2]⁺=639.4.

### Synthesis of Conjugate 172a

Int-305 (1.2 g, 0.95 mmol) in DMF (10 mL) was added to SEQ ID NO: 80 (3.5 g in 250 mL PBS 7.4, 0.060 mmol) in PBS at pH 7.4 as described in the general conjugation procedure. Maldi TOF analysis of the purified final product gave an average mass of 63,976 Da. (DAR=7.7). Yield 2.7 g, 77%.

### Synthesis of Conjugate 172b

Int-305 (41 mg, 0.0036 mmol) in DMF (0.4 mL) was added to Palivizumab full length antibody (53 mg in mL PBS 7.4, 0.00036 mmol) in PBS at pH 7.4 as described in the general conjugation procedure. Maldi TOF analysis of the purified final product gave an average mass of 155,413 Da. (DAR=7.3). Yield 26.9 mg, 50.7%.

### Synthesis of Conjugate 172c

Int-305 (33 mg, 0.026 mmol) in DMF (0.6 mL) was added to SEQ ID NO: 116 (120 mg in 6.7 mL PBS 7.4, 0.002 mmol) in PBS at pH 7.4 as described in the general conjugation procedure. Maldi TOF analysis of the purified final product gave an average mass of 64,360 Da. (DAR=8.2). Yield 84.5 mg, 70.4%.

Synthesis of Conjugate 172c (batch 2)

Int-305 (22 mg, 0.017 mmol) in DMF (0.6 mL) was added to SEQ ID NO: 116 (120 mg in 6.7 mL PBS 7.4, 0.002 mmol) in PBS at pH 7.4 as described in the general conjugation procedure. Maldi TOF analysis of the purified final product gave an average mass of 61,509 Da. (DAR=5.7). Yield 88.1 mg, 73.4%.

Synthesis of Conjugate 172c (batch 3)

Int-305 (33 mg, 0.026 mmol) in DMF (0.6 mL) was added to SEQ ID NO: 116 (120 mg in 6.7 mL PBS 7.4, 0.002 mmol) in PBS at pH 7.4 as described in the general conjugation procedure. Maldi TOF analysis of the purified final product gave an average mass of 64,360 Da. (DAR=8.2). Yield 84.5 mg, 70.4%.

Synthesis of Conjugate 172c (batch 4)

Int-305 (44 mg, 0.034 mmol) in DMF (0.6 mL) was added to SEQ ID NO: 116 (120 mg in 6.7 mL PBS 7.4, 0.002 mmol) in PBS at pH 7.4 as described in the general conjugation procedure. Maldi TOF analysis of the purified final product gave an average mass of 66,419 Da. (DAR=10). Yield 84.3 mg, 70.3%.

Synthesis of Conjugate 172c (batch 5)

Int-305 (55 mg, 0.043 mmol) in DMF (0.6 mL) was added to SEQ ID NO: 116 (120 mg in 6.7 mL PBS 7.4, 0.002 mmol) in PBS at pH 7.4 as described in the general conjugation procedure. Maldi TOF analysis of the purified final product gave an average mass of 67,921 Da. (DAR=11.3). Yield 85.3 mg, 71.1%.

Synthesis of Conjugate 172c (batch 6)

Int-305 (66 mg, 0.052 mmol) in DMF (0.6 mL) was added to SEQ ID NO: 116 (120 mg in 6.7 mL PBS 7.4, 0.002 mmol) in PBS at pH 7.4 as described in the general conjugation procedure. Maldi TOF analysis of the purified final product gave an average mass of 70,124 Da. (DAR=13.3). Yield 86.1 mg, 71.8%.

Synthesis of Conjugate 172c (batch 9)

SEQ ID NO: 116 (17.2 g, 0.31 mmol) was buffer exchanged into sodium bicarbonate buffer (419 mL, pH 9.5, 0.1 M, 8 DV, 0.75 I/min) and Int-305 (4.36 g, 3.41 mmol) in DMF (21 mL) was added and the reaction mixture stirred for 3 h. After target DAR was achieved, buffer exchange by tangential flow filtration (0.1 M NaHCO₃, pH 9.5, 0.1 M NaCl, 7 DV, 0.75 L/min) followed by buffer exchange with histidine buffer (20 mM Histidine, pH 5.5, 8 DV, 0.75 I/min) provided the purified desired product. Maldi TOF analysis of the purified final product gave an average mass of 64,080 Da. (DAR=7.9). Yield 19 g, 94%.

### Synthesis of Conjugate 172d

Int-305 (13 mg, 0.01 mmol) in DMF (0.2 mL) was added to bovine serum albumin (BSA) antibodies (60 mg in 6.0 mL PBS 7.4, 0.0009 mmol) in PBS at pH 7.4 as described in the general conjugation procedure. Maldi TOF analysis of the purified final product gave an average mass of 76,952, Da. (DAR=9.3). Yield 36 mg, 60%.

### Synthesis of Int-294

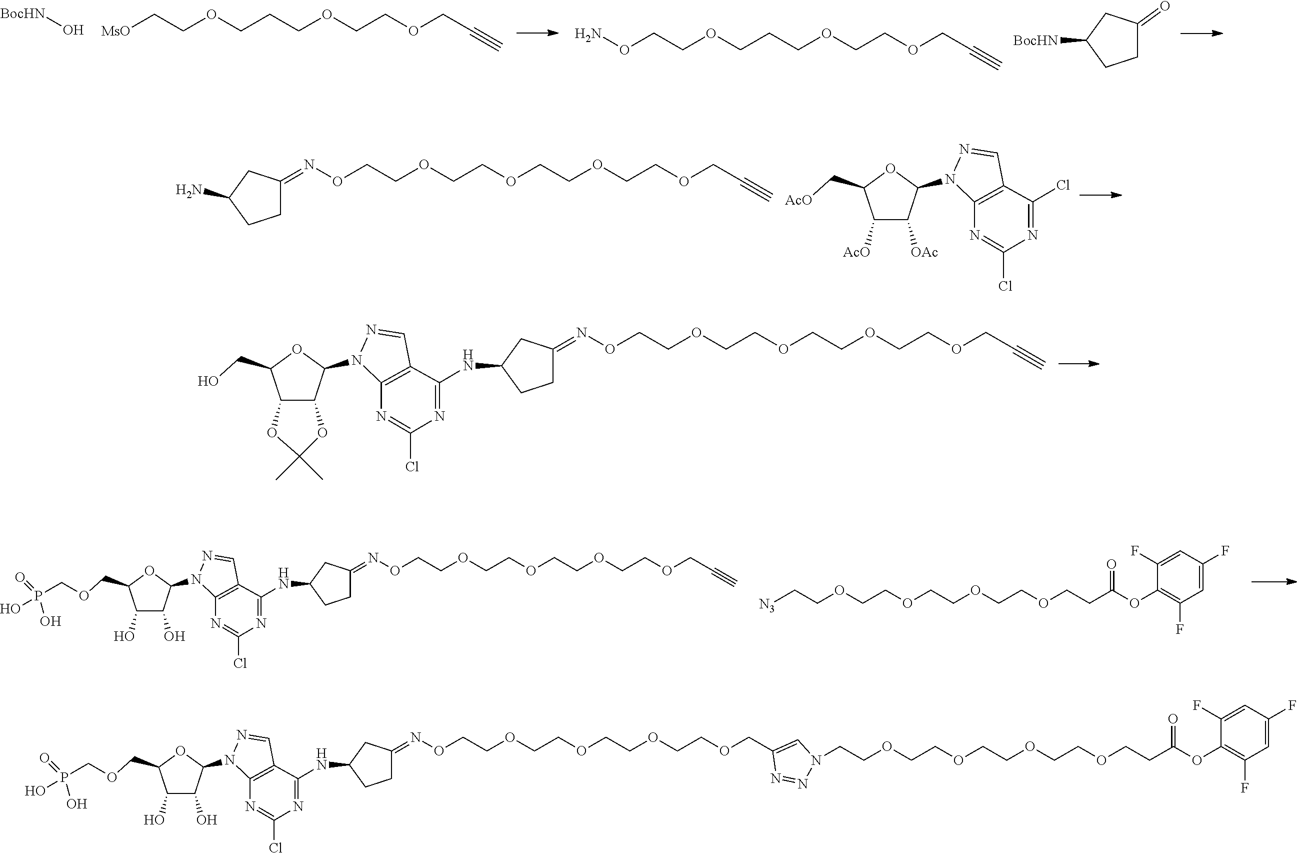

### Step a

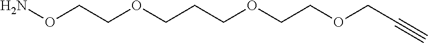

To a mixture of N-Boc aminooxy (2.3 g, 18 mmol) and propargyl-PEG4-mesyl ester (4.6 g, 15 mmol) was added DBU (2.7 g, 18 mmol). The neat reaction was stirred at room temperature overnight. The mixture was dissolved in DCM, washed with H₂O, NaHCO₃ (10%) and brine. The organic layer was separated and dried over sodium sulfate, filtered, and concentrated in vacuo to provide crude, which was purified by flash chromatography (DCM: 10% MeOH in DCM) to yield product. Yield 4.2 g, 80%. LCMS: [M+H]⁺=317.4. Intermediate N-Boc derivative was dissolved in DCM (10 mL) and TFA (4 mL). The resulting mixture was stirred for 4 h at room temperature then solvent was removed under reduced pressure and dried under vacuum to yield the desired compound as its TFA salt. The product was a colorless oil. Yield 3.2 g, 100%. Ion(s) found by LCMS: [M+H]⁺=217.1.

### Step b

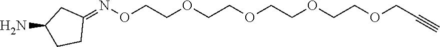 [SMILES: C#CCOCCOCCOCCOCCO/N=C1\CC[C@@H](N)C1]

To a solution of N-Boc cyclopentyl (1.0 g, 4.8 mmol) and aminooxy-PEG4-propargyl ether (1.8 g, 5.3 mmol) in DMF (10 ml) was added DIPEA (2.5 ml, 14 mmol). The solution was stirred at room temperature for 20 min and purified by semi-preparative HPLC (5% to 95% acetonitrile and water, using 0.1% TFA as modifier). Yield 1.55 g, 74%. LCMS: [M+H]⁺=428.2. Intermediate N-Boc derivative was dissolved in DCM (10 mL) and TFA (4 mL). The resulting mixture was stirred for 3 h at room temperature then solvent was removed under reduced pressure and dried under vacuum to yield the desired compound as its TFA salt. The product was a colorless oil. Yield 1.2 g, 100%. Ion(s) found by LCMS: [M+H]⁺=328.1.

### Step c

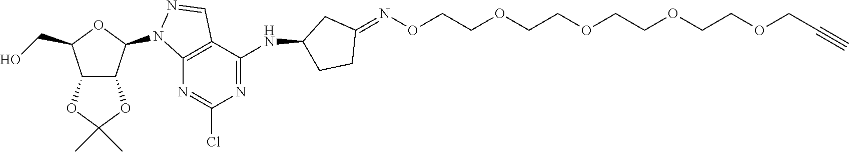 [SMILES: C#CCOCCOCCOCCOCCO/N=C1\CC[C@@H](Nc2nc(Cl)nc3c2cnn3[C@@H]2O[C@H](CO)[C@H]3OC(C)(C)O[C@H]32)C1]

A solution of triacetate intermediate B (174 mg, 0.4 mmol), step-a product (140 mg, 0.32 mmol) and triethylamine (0.068 mL, 0.48 mmol) in anhydrous EtOH (1 mL) was stirred at room temperature for 30 min. The solution was concentrated, and the residue was dissolved in 7 M NH₃/MeOH (5 mL). The reaction was stirred at room temperature overnight and mixture was concentrated to dryness under reduced pressure, then dissolved in acetone (0.5 mL). 2,2-dimethoxypropane (0.5 mL, 3.9 mmol) and p-TsOH (77 mg, 0.4 mmol) was added, and the mixture was stirred overnight at room temperature. The reaction mixture was concentrated to dryness under reduced pressure and purified by flash chromatography (DCM: 10% MeOH in DCM) to yield product. The product was a white foam. Yield 120 mg, 56%. Ion(s) found by LCMS: [M+H]⁺=653.2.

### Step d

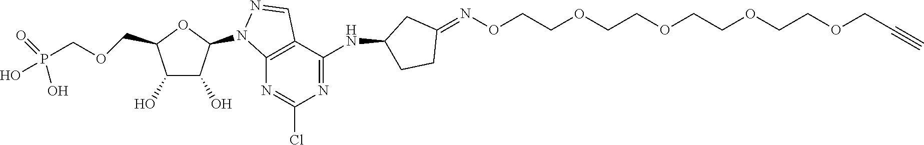 [SMILES: C#CCOCCOCCOCCOCCO/N=C1\CC[C@@H](Nc2nc(Cl)nc3c2cnn3[C@@H]2O[C@H](COCP(=O)(O)O)[C@@H](O)[C@H]2O)C1]

A solution of acetonide derivative from previous step (100 mg, 0.15 mmol) and [bis(tert-butoxy)carbonyl]methyl (trifluoromethyl)sulfonate (163 mg, 0.45 mmol) in dry DMF (1.5 mL) was cooled using ice-water bath. 60% NaH in mineral oil (36 mg, 0.9 mmol) was added to this and stirred at room temperature for 2 h. The reaction mixture was quenched with saturated ammonium chloride and extracted with EtOAc. The organic phase was washed with brine and dried over sodium sulfate concentrated to give crude product which was used in the next step without purification. Crude product was dissolved in 50% aqueous TFA (5 mL) and stirred at room temperature for 12 h, concentrated and the residue was purified by semi-preparative HPLC (10-100% can: H₂O, 0.1% TFA modifier). The product was a white solid. Yield 92 mg, 84%. Ion(s) found by LCMS: [M+H]⁺=707.2.

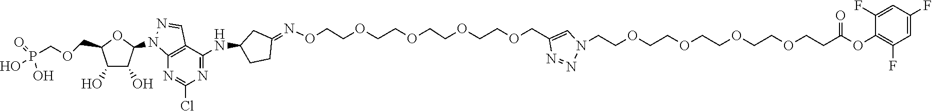 [SMILES: O=C(CCOCCOCCOCCOCCn1cc(COCCOCCOCCOCCO/N=C2\CC[C@@H](Nc3nc(Cl)nc4c3cnn4[C@@H]3O[C@H](COCP(=O)(O)O)[C@@H](O)[C@H]3O)C2)nn1)Oc1c(F)cc(F)cc1F]

A solution of the product from the previous step (30 mg, 0.042 mmol) and intermediate A (24 mg, 0.057 mmol) in DMF (0.5 mL) was cooled to 0° C., and treated with a pre-mixed solution of a solution of copper(II) sulfate (0.002 mg, 0.01 mmol), sodium ascorbate (8 mg, 0.042 mmol), and THPTA (10 mg, 0.021 mmol) dissolved in water (0.5 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a white solid. Yield 30 mg, 62%. Ion(s) found by LCMS [(M+2H)/2]⁺=564.5.

### Synthesis of Conjugate 161

Int-294 (6.1 mg, 0.005 mmol) in DMF (0.4 mL) was added to SEQ ID NO: 80 (50 mg in 3.8 mL PBS 7.4, 0.000905 mmol) in PBS at pH 7.4 as described in the general conjugation procedure. Maldi TOF analysis of the purified final product gave an average mass of 58,935 Da. (DAR=3.8). Yield 31.1 mg, 62.1%.

### Synthesis of Int-295

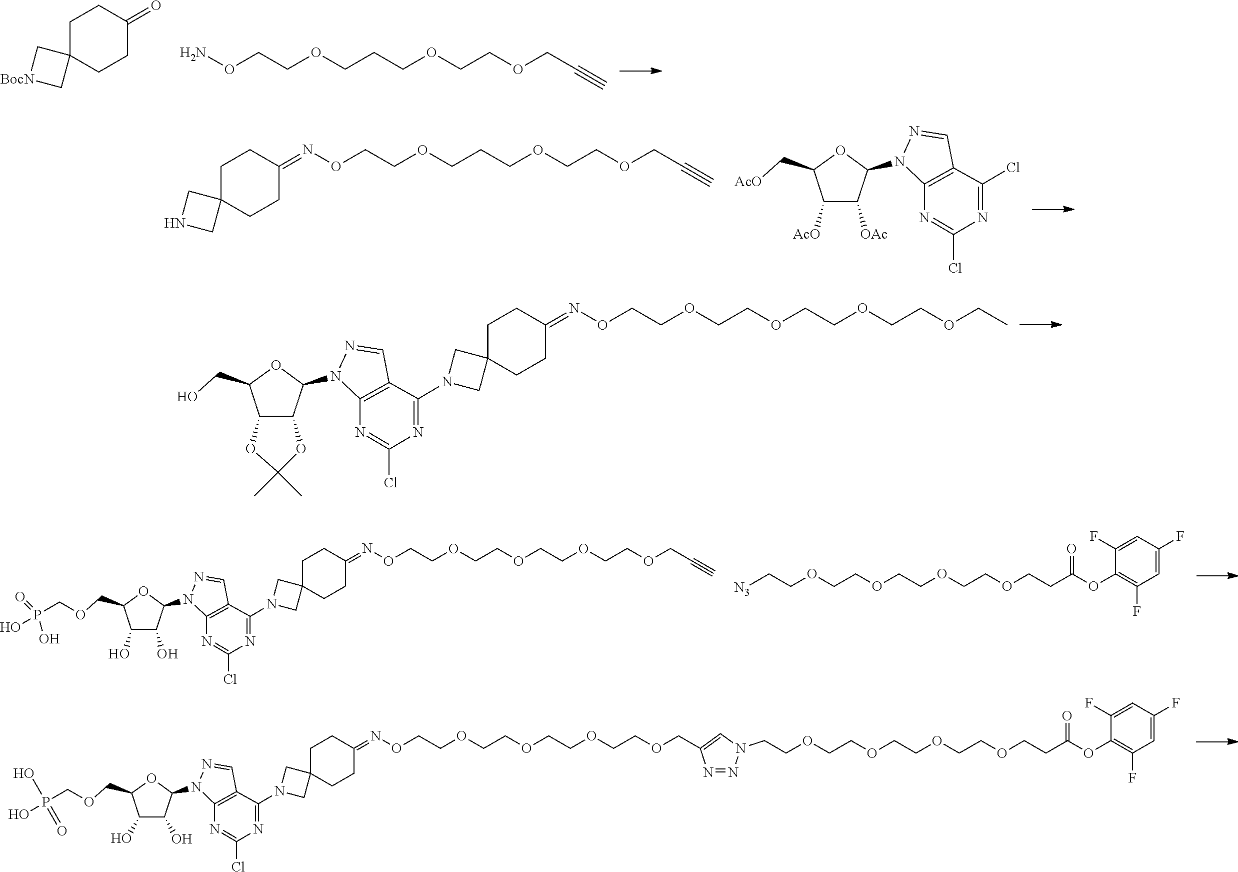

### Step a

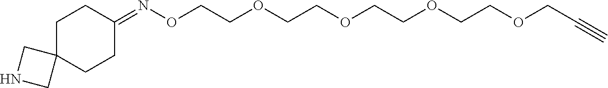 [SMILES: C#CCOCCOCCOCCOCCON=C1CCC2(CC1)CNC2]

To a solution of N-Boc spiro azetidine (142 mg, 0.57 mmol) and aminooxy-PEG4-propargyl ether (200 mg, 0.57 mmol) in DMF (5 ml) was added DIPEA (0.6 ml, 3.4 mmol). The solution was stirred at room temperature for 20 min and purified by semi-preparative HPLC (5% to 95% acetonitrile and water, using 0.1% TFA as modifier). Yield 230 mg, 85%. LCMS: [M+H]⁺=468.2. Intermediate N-Boc derivative was dissolved in DCM (5 mL) and TFA (2 mL). The resulting mixture was stirred for 4 h at room temperature then solvent was removed under reduced pressure and dried under vacuum to yield the desired compound as its TFA salt. The product was a colorless oil. Yield 210 g, 100%. Ion(s) found by LCMS: [M+H]⁺=368.1.

### Step b

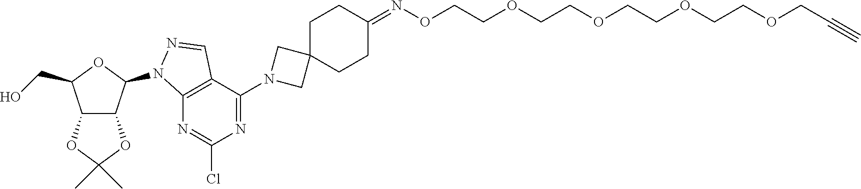 [SMILES: C#CCOCCOCCOCCOCCON=C1CCC2(CC1)CN(c1nc(Cl)nc3c1cnn3[C@@H]1O[C@H](CO)[C@H]3OC(C)(C)O[C@H]31)C2]

A solution of triacetate intermediate B (304 mg, 0.54 mmol), step-a product (210 mg, 0.5 mmol) and triethylamine (0.1 ml, 0.74 mmol) in anhydrous EtOH (1 mL) was stirred at room temperature for 30 min. The solution was concentrated and the residue was dissolved in 7 M NH3/MeOH (5 ml). The reaction was stirred at room temperature overnight and mixture was concentrated to dryness under reduced pressure, then dissolved in acetone (1 mL). 2,2-dimethoxypropane (1 mL, 6 mmol) and p-TsOH (120 mg, 0.6 mmol) was added and the mixture was stirred overnight at room temperature for 2 hours. The reaction mixture was concentrated to dryness under reduced pressure and purified by flash chromatography (DCM: 10% MeOH in DCM) to yield product. The product was a white foam. Yield 233 mg, 67%. Ion(s) found by LCMS: [M+H]⁺=693.2.

### Step c

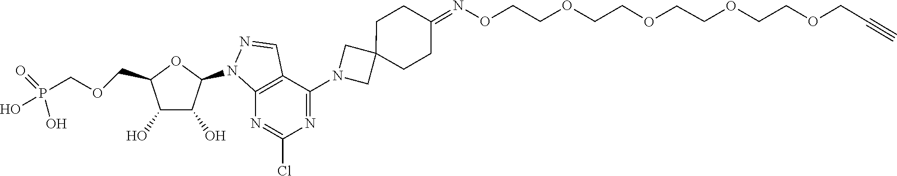 [SMILES: C#CCOCCOCCOCCOCCON=C1CCC2(CC1)CN(c1nc(Cl)nc3c1cnn3[C@@H]1O[C@H](COCP(=O)(O)O)[C@@H](O)[C@H]1O)C2]

A solution of acetonide derivative from previous step (233 mg, 0.33 mmol) and [bis(tert-butoxy)carbonyl]methyl (trifluoromethyl)sulfonate (360 mg, 1.0 mmol) in dry DMF (3 mL) was cooled using ice-water bath. 60% NaH in mineral oil (80 mg, 2.0 mmol) was added to this and stirred at room temperature for 2 h. The reaction mixture was quenched with saturated ammonium chloride and extracted with EtOAc. The organic phase was washed with brine and dried over sodium sulfate concentrated to give crude product which was used in the next step without purification. Observed LCMS: Crude product was dissolved in 50% aqueous TFA (5 mL) and stirred at room temperature for 12 h, concentrated and the residue was purified by semi-preparative HPLC (10-100% ACN: H₂O, 0.1% TFA modifier). The product was a white solid. Yield 95 mg, 37%. Ion(s) found by LCMS: [M+H]⁺=747.2.

### Step d

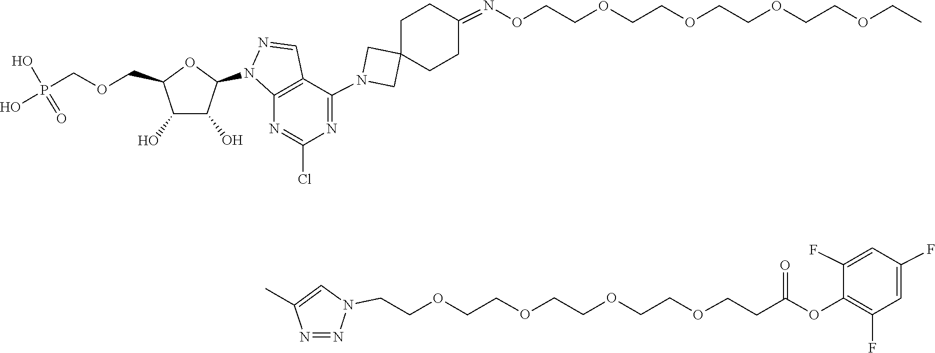

To a solution of the product from the previous step (30 mg, 0.04 mmol) and intermediate A (22 mg, 0.05 mmol) in DMF (0.5 ml) was cooled to 0° C., and treated with a pre-mixed solution of a solution of copper(II) sulfate (0.002 mg, 0.01 mmol), sodium ascorbate (8 mg, 0.042 mmol), and THPTA (10 mg, 0.021 mmol) dissolved in water (0.5 mL) then stirred for 5 minutes at the same temperature, and then gradually warmed to room temperature and stirred for 15 minutes. When the reaction was complete (by HPLC) the product was purified by reversed phase HPLC (10% to 100% ACN/water, 0.1% TFA modifier). The product was a white solid. Yield 31 mg, 66%. Ion(s) found by LCMS [(M+2H)/2]⁺=584.6.

### Synthesis of Conjugate 162

Int-295 (8.4 mg, 0.007 mmol) in DMF (0.5 mL) was added to SEQ ID NO: 80 (50 mg in 3.8 mL PBS 7.4, 0.000905 mmol) in PBS at pH 7.4 as described in the general conjugation procedure. Maldi TOF analysis of the purified final product gave an average mass of 61,089 Da. (DAR=5.7). Yield 28.4 mg, 56.8%.

### Synthesis of Int-297

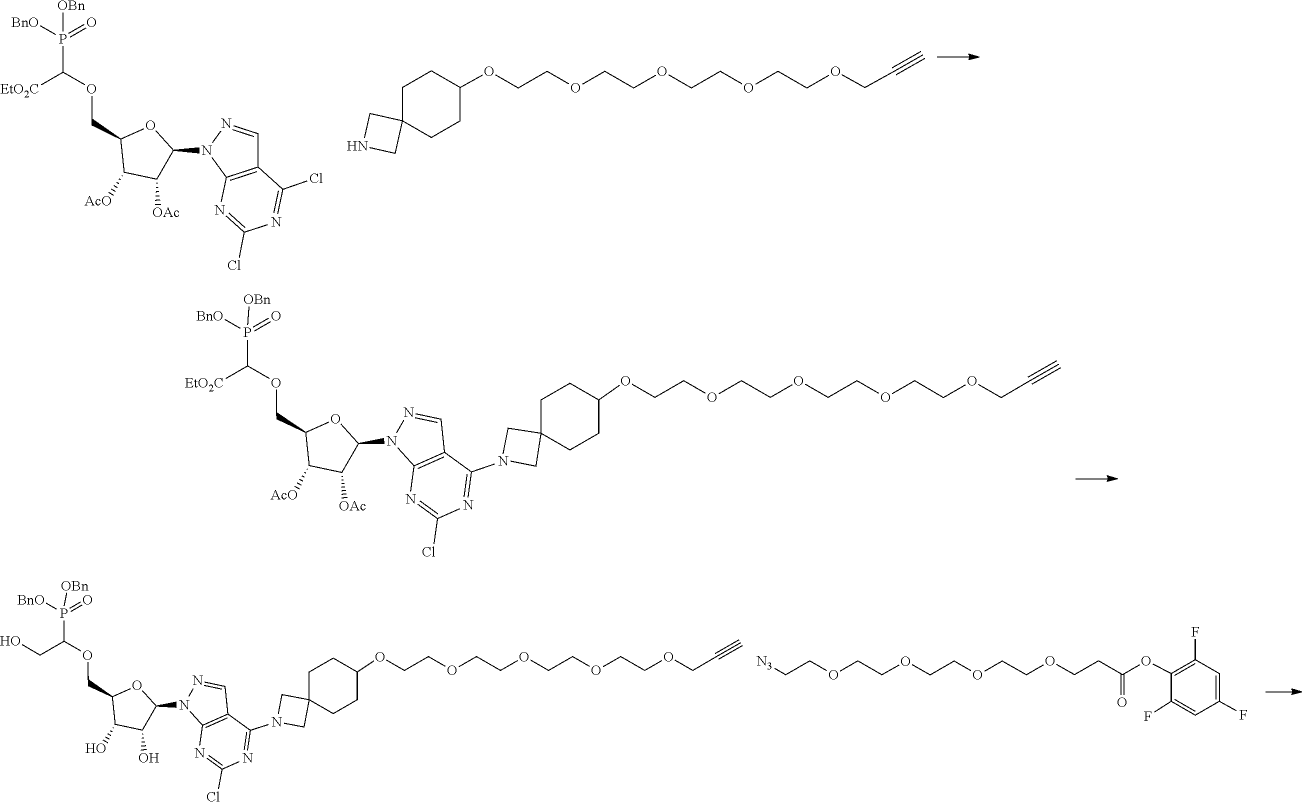

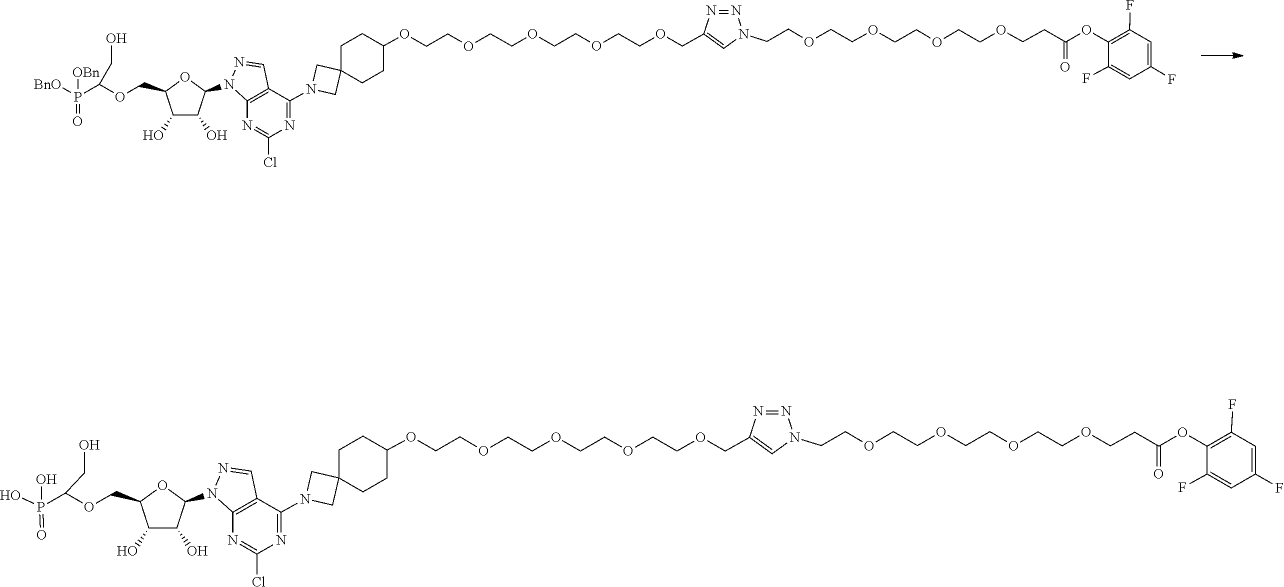 [SMILES: O=C(CCOCCOCCOCCOCCn1cc(COCCOCCOCCOCCOC2CCC3(CC2)CN(c2nc(Cl)nc4c2cnn4[C@@H]2O[C@H](COC(CO)P(=O)(OCc4ccccc4)OCc4ccccc4)[C@@H](O)[C@H]2O)C3)nn1)Oc1c(F)cc(F)cc1F ; O=C(CCOCCOCCOCCOCCn1cc(COCCOCCOCCOCCOC2CCC3(CC2)CN(c2nc(Cl)nc4c2cnn4[C@@H]2O[C@H](COC(CO)P(=O)(O)O)[C@@H](O)[C@H]2O)C3)nn1)Oc1c(F)cc(F)cc1F]

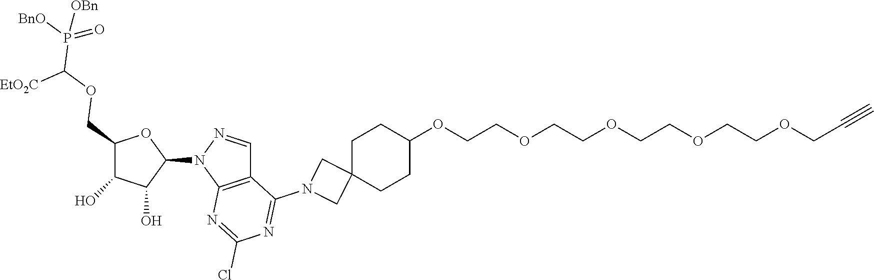 [SMILES: C#CCOCCOCCOCCOCCOC1CCC2(CC1)CN(c1nc(Cl)nc3c1cnn3[C@@H]1O[C@H](COC(C(=O)OCC)P(=O)(OCc3ccccc3)OCc3ccccc3)[C@@H](O)[C@H]1O)C2]

The 2,4-Dichlorppyrimidine derivative (375 mg, 0.5 mmol) described in step d of Synthesis of Int-291, the amine (266 mg, 0.75 mmol) described in step a synthesis of Int-259, and DIEA (0.17 mL, 1 mmol) in MeOH (5 mL) was heated at 50° C. for 1 hour. After complete consumption of the starting materials, the reaction mixture was cooled to room temperature and volatiles were removed by rotatory evaporation. The crude residue adsorbed on silica gel and purified by flash column chromatography (DCM: 10% MeOH in DCM) to afford the desired product as a white foam. Yield 421 mg, 79%. Ion(s) found by LCMS: [M+H]⁺=1070.2.

### Step b

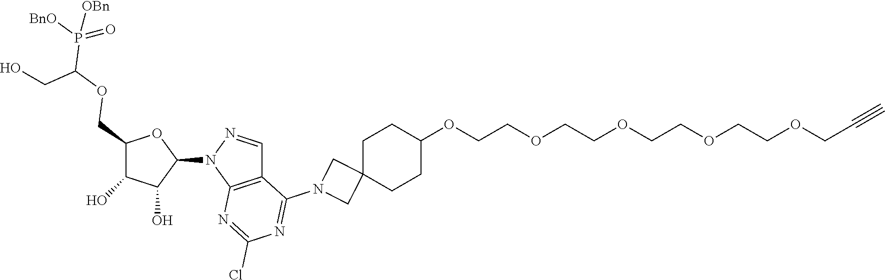 [SMILES: C#CCOCCOCCOCCOCCOC1CCC2(CC1)CN(c1nc(Cl)nc3c1cnn3[C@@H]1O[C@H](COC(CO)P(=O)(OCc3ccccc3)OCc3ccccc3)[C@@H](O)[C@H]1O)C2]

To a solution of compound from previous step (400 mg, 0.37 mmol) in ethanol (15 mL) that had been cooled to 0° C. using an ice bath, was added calcium dichloride (83 mg, 0.74 mmol) followed by sodium borohydride (28 mg, 0.74 mmol). The mixture was warmed to ambient temperature, where it was stirred for additional 1 hour, before being placed back into an ice bath. Once cooled, the reaction was quenched with 1 N HCl (3 mL), and then diluted with EtOAc (30 mL). The flask was removed from the bath and allowed to stir at room temperature for 20 minutes. The organic layer was washed with additional 1 N HCl and the combined aqueous layers were extracted with EtOAc (2×10 mL). The combined organic layers were then washed with brine, dried over sodium sulfate, filtered, and concentrated under reduced pressure. The resulting crude residue was purified by RPLC (10% to 100% ACN/water, 0.1% TFA modifier) to yield the title compound as white solid. Yield 98 mg, 28%. Ion(s) found by LCMS: [M+H]⁺=944.2.

### Step c

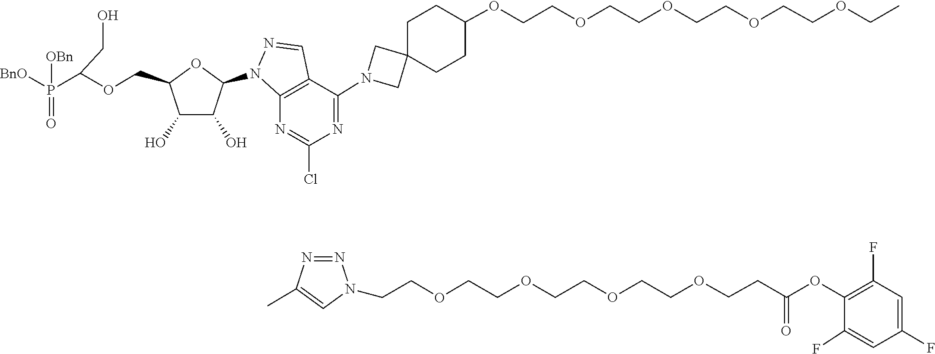

To a solution of product from the previous step (80 mg, 0.085 mmol) and intermediate A (36 mg, 0.084 mmol). dissolved in DMF:H₂O (1:3, 1.5 mL) were cooled to 0° C. To this a pre-mixed solution of a solution of copper(II) sulfate (1.3 mg, 0.008 mmol), sodium ascorbate (50 mg, 0.254 mmol), and BTTA (7 mg, 0.017 mmol) dissolved in water (0.5 mL) was added and stirred for 5 minutes at the same temperature and gradually warmed to room temperature and stirred for an additional 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 68 mg, 59%. Ion(s) found by LCMS [(M+2H)/2]⁺=683.4.

### Step d

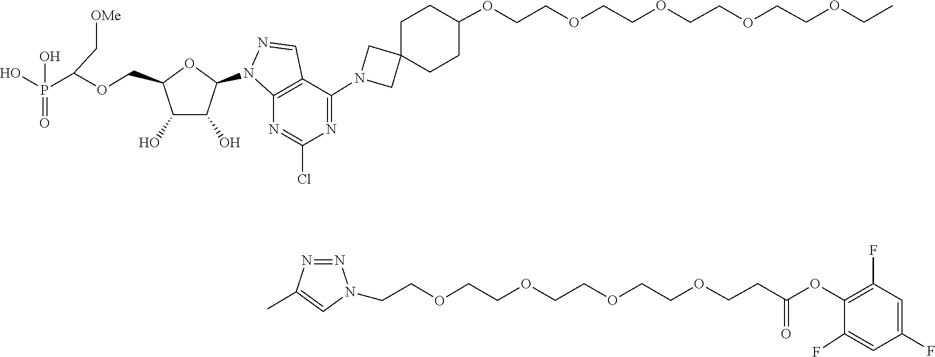

To a solution of compound from previous step (68 mg, 0.05 mmol) in MeOH (5 mL) at ambient temperature was added 5% Pd/C (21 mg). The resulting mixture was stirred at room temperature under an atm of hydrogen for 30 minutes. Reaction mixture was filtered through a short pad of celite. Concentration of the filtrate followed by purification by RPLcanACN: H₂O, 0.1% TFA modifier) yielded the title compound as a white solid. Yield 40 mg, 68%. Ion(s) found by LCMS [(M+2H)/2]⁺=593.2.

### Synthesis of Conjugate 164

Int-297 (22 mg, 0.0174 mmol) was added to SEQ ID NO: 80 (120 mg, 9.3 mL in PBS at pH 8.4) as described in the general conjugation procedure. Maldi TOF analysis of the purified final product gave an average mass of 59,210 Da. (DAR=3.8). Yield 89 mg, 69%.

### Synthesis of Int-298

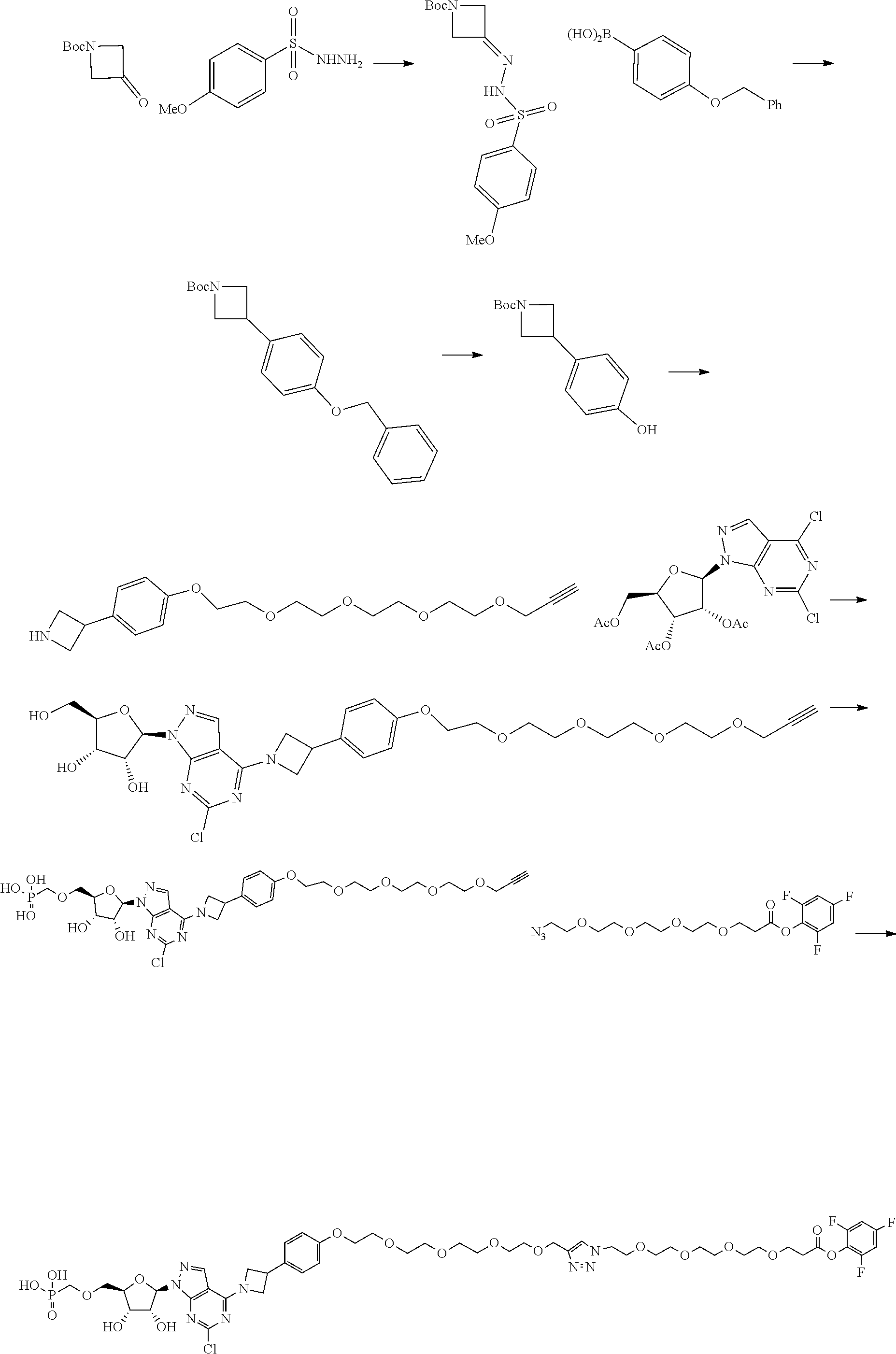

### Step a

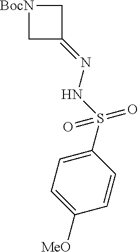 [SMILES: COc1ccc(S(=O)(=O)NN=C2CN(C(=O)OC(C)(C)C)C2)cc1]

To a solution of sulfonylhydrazone (523 mg, 3.06 mmol) in MeOH (10 mL) was added 1-Boc-3-azetidone (618 mg, 3.06 mmol). The reaction mixture was stirred at room temperature until complete conversion was observed by LCMS (1 hour). Solvents were removed in vacuo to give sulfonylhydrazone which was used for the next step without any purification. The product was a white solid. Yield 1 g, 92%. Ion(s) found by LCMS: [M-^{t}Bu+H]⁺=256.2.

### Step b

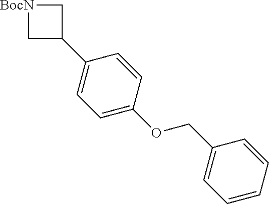 [SMILES: CC(C)(C)OC(=O)N1CC(c2ccc(OCc3ccccc3)cc2)C1]

Sulfonylhydrazone derivative from previous step (1 g, 2.8 mmol), 4-(benzyloxy)phenylboronic acid (962 mg, 4.22 mmol), and cesium carbonate (2.75 g, 4.22 mmol) were placed in an oven-dried tube in vacuo for 30 minutes. The tube was backfilled with nitrogen followed by the addition of dry degassed 1,4-Dioxane (50 mL). This tube was sealed and heated to 110° C. for 18 hours before being cooled to room temperature, quenched with saturated NaHCO₃ solution (10 mL), and extracted with EtOAc (3×25 mL). The combined organic layer was dried over sodium sulfate, and solvents were removed in vacuo to give a residue, which was purified by flash column chromatography (hexanes: EtOAc). Product was a light yellow viscous oil. Yield 750 mg, 79%. Ion(s) found by LCMS: [M−^{t}Bu+H]⁺=284.5.

### Step c

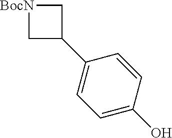 [SMILES: CC(C)(C)OC(=O)N1CC(c2ccc(O)cc2)C1]

The compound from step b (750 mg, 2.21 mmol) was dissolved in MeOH (20 mL) and purged with nitrogen. To this, 10 mol % Pd/C (100 mg) was added and the resulting mixture was stirred at room temperature under an atmosphere of hydrogen overnight. Reaction mixture was filtered through a short pad of celite and washed with MeOH. Evaporation of the filtrate yielded crude product which was used for the next step without any purification. The product was an off-white solid. Yield 360 mg, 65%. Ion(s) found by LCMS: [M−^{t}Bu+H]⁺=194.2.

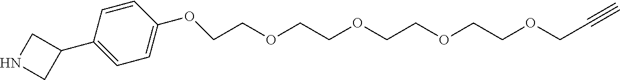 [SMILES: C#CCOCCOCCOCCOCCOc1ccc(C2CNC2)cc1]

The compound from the previous step (360 mg, 1.44 mmol), propargylPEG4mesylate (448 mg, 1.44 mmol) and potassium carbonate (399 mg, 2.89 mmol) were dissolved in acetonitrile (20 mL) and heated at reflux overnight and cooled to room temperature. Solvent was removed by rotary evaporation to give crude product, which was re-dissolved in ethyl acetate and washed with water. The organic phase was dried over sodium sulfate, and solvents were removed in vacuo to give a residue, which was purified by reverse phase HPLC (ACN:H₂O, 0.1% TFA modifier). The product was a colorless viscous oil. 500 mg, 75%. Ions (s) found by LCMS: [M−^{t}Bu+H]⁺=408.2. N-Boc deprotected compound (190 mg, 0.41 mmol) dissolved in dioxane and treated with 4M HCl (5 mL) at room temperature for stirred 6h. Solvent was removed and dried to yield the compound. The product was a colorless viscous oil. Yield 174 mg, Quantitative yield. Ions found by LCMS: [M+H]⁺=364.2.

### Step e

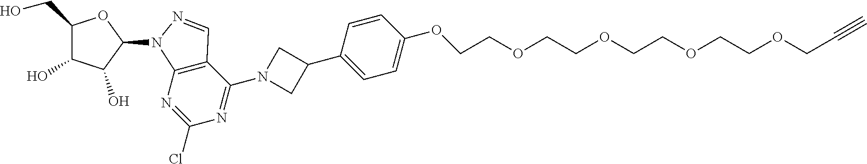 [SMILES: C#CCOCCOCCOCCOCCOc1ccc(C2CN(c3nc(Cl)nc4c3cnn4[C@@H]3O[C@H](CO)[C@@H](O)[C@H]3O)C2)cc1]

A mixture of intermediate B (179 mg, 0.4 mmol), the amine from previous step (175 mg, 0.48 mmol) and diisopropylamine (0.11 mL, 0.8 mmol) in MeOH (6 mL) was heated at 50° C. for 2 hours. Reaction mixture was cooled to room temperature and excess solvent was removed under reduced pressure to yield the crude product which was used for the next step without any purification. Ions found by LCMS: [M+H]⁺=774.2. Crude product was re-dissolved in MeOH (5 mL) and solid potassium carbonate (193 mg, 1.4 mmol) was added and the reaction stirred at room temperature for 2 hours. Solvent was removed under reduced pressure. The crude residue was redissolved in EtOAc (50 mL) and washed with water and brine. The organic layer was dried over sodium sulfate and solvent was removed under reduced pressure to yield the product which was further purified by HPLC (ACN:H₂O, 0.1% TFA modifier). The product was an off-white foam. Yield 176 mg, 68%. Ions found by LCMS: [M+H]⁺=648.2.

### Step f

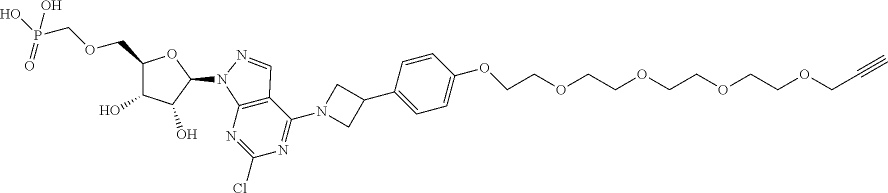 [SMILES: C#CCOCCOCCOCCOCCOc1ccc(C2CN(c3nc(Cl)nc4c3cnn4[C@@H]3O[C@H](COCP(=O)(O)O)[C@@H](O)[C@H]3O)C2)cc1]

To a solution of the triol from previous step (140 mg, 0.216 mmol) and 2,2-dimethoxypropane (67 mg, 0.648 mmol) in acetone (10 mL) at room temperature was added toluene sulfonic acid (37 mg, 0.216 mmol). The reaction was stirred for two hours then concentrated under reduced pressure. The crude material was dissolved in EtOAc (20 mL) and washed with saturated NaHCO₃. The organic layer was dried over sodium sulfate, filtered, and concentrated in vacuo to provide crude acetonide derivative, which was purified by flash chromatography (DCM: 10% MeOH in DCM) to yield corresponding acetonide derivative as a white foam. Yield 108 mg, 73%. Ion(s) found by LCMS: [M+H]⁺=688.2. A solution of the acetonide derivative (108 mg, 0.157 mmol) and [bis(tert-butoxy)carbonyl]methyl (trifluoromethyl)sulfonate (168 mg, 0.471 mmol) in dry DMF (3 mL) was cooled using an ice-water bath. 60% NaH in mineral oil (40 mg, 0.942 mmol) was added to this and gradually warmed to room temperature and stirred for 1 hour. The reaction mixture was quenched with saturated ammonium chloride then extracted with EtOAc. The organic phase was washed with brine, dried over sodium sulfate, and concentrated to give crude product which was used in the next step without purification. Ion(s) found by LCMS: [M+H]⁺=894.2. Crude product was dissolved in 50% aqueous TFA (10 mL) and stirred at room temperature for 12 hours, concentrated, and purified by semi-preparative HPLC (10% to 100% ACN/water, 0.1% TFA modifier) The product was a white solid. Yield 68 mg, 59%. Ion(s) found by LCMS: [M+H]⁺=742.2.

### Step g

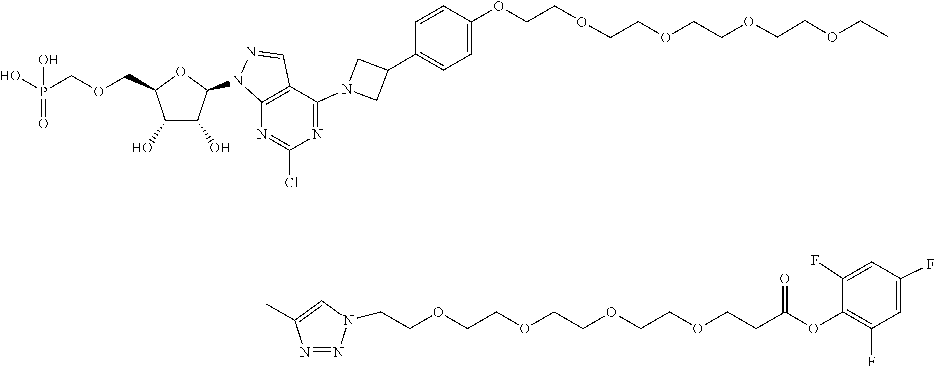

To a solution of product from the previous step (40 mg, 0.0539 mmol) and intermediate A (22 mg, 0.0539 mmol) dissolved in DMF:H₂O (1:3, 1.5 mL) were cooled to 0° C. To this a pre-mixed solution of a solution of copper(II) sulfate (1 mg, 0.005 mmol), sodium ascorbate (32 mg, 0.163 mmol), and BTTA (5 mg, 0.011 mmol) dissolved in water (0.5 mL) was added and stirred for 5 minutes at the same temperature and gradually warmed to room temperature and stirred for an additional 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 43 mg, 69%. Ion(s) found by LCMS [(M+2H)/2]⁺=582.2.

### Synthesis of Conjugate 165

Int-298 (16.8 mg, 0.01448 mmol) was added to SEQ ID NO: 80 (100 mg, 7.75 mL in PBS at pH 8.4) as described in the general conjugation procedure. Maldi TOF analysis of the purified final product gave an average mass of 63,362 Da. (DAR=8). Yield 66 mg, 66%.

### Synthesis of Int-299

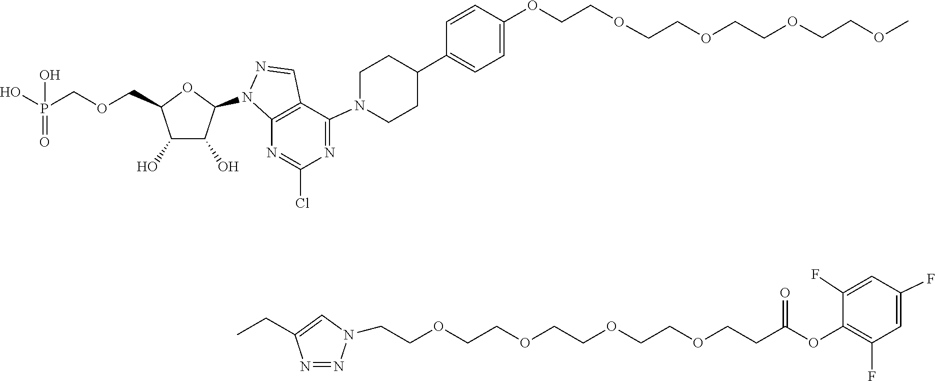

The title compound was prepared analogously to Int-298 where 1-Boc-3-azetidone was replaced with tert-butyl 4-oxopiperidinecarboxylate. The product was a white solid. Yield 39 mg, 63%. Ion(s) found by LCMS: [(M+2H)/2]⁺=596.2.

### Synthesis of Conjugate 166

Int-299 (17.2 mg, 0.01448 mmol) was added to SEQ ID NO: 80 (100 mg, 7.75 mL in PBS at pH 8.4) as described in the general conjugation procedure. Maldi TOF analysis of the purified final product gave an average mass of 62,121 Da. (DAR=6.6). Yield 66 mg, 66%.

### Synthesis of Int-318

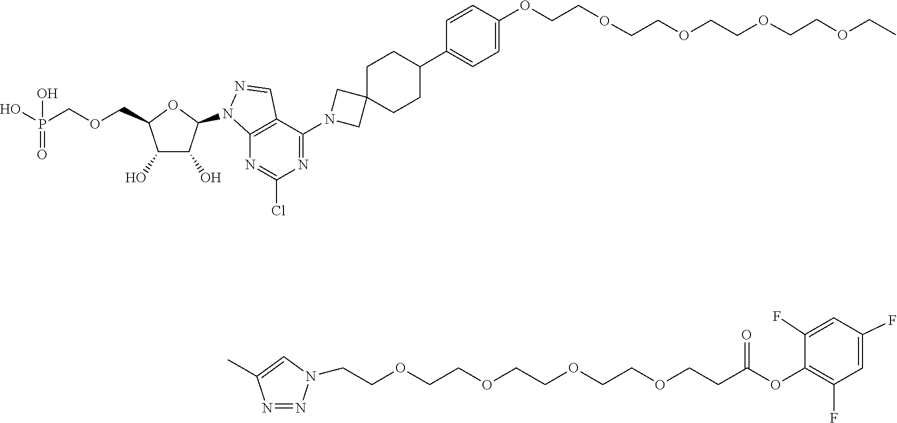

The title compound was prepared analogously to Int-298, where 1-Boc-3-azetidone was replaced with tert-butyl 7-oxo-2-azaspiro[3.5]nonane-2-carboxylate. The product was a white solid. Yield 7 mg, 58%. Ion(s) found by LCMS [(M+2H)/2]⁺=616.2.

### Synthesis of Conjugate 185

Int-318 (7 mg, 0.00579 mmol) in DMF (0.5 mL) was added to SEQ ID NO: 80 (40 mg, 3.1 mL in PBS at pH 8.4) as described in the general conjugation procedure. Maldi TOF analysis of the purified final product gave an average mass of 59,749 Da. (DAR=4.2). Yield 18 mg, 45%.

### Synthesis of Int-320

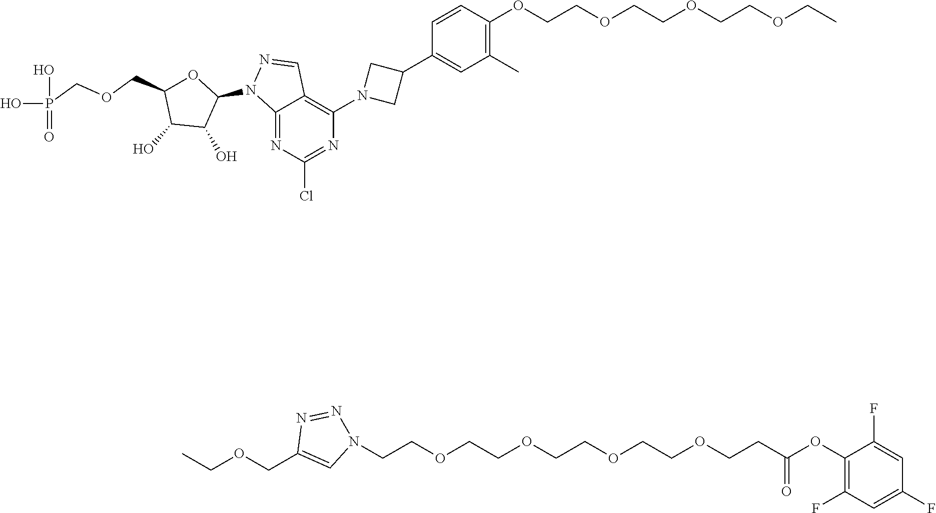

The title compound was prepared analogously to Int-298, where 4-(benzyloxy)phenylboronic acid was replaced with [4-(benzyloxy)-3-methylphenyl]boronic acid. The product was a white solid. Yield 42 mg, 54%. Ion(s) found by LCMS [(M+2H)/2]⁺=589.2.

### Synthesis of Conjugate 187

Int-320 (25.6 mg, 0.0217 mmol) in DMF (0.5 mL) was added to SEQ ID NO: 80 (150 mg, 7.75 mL in PBS at pH 8.4) as described in the general conjugation procedure. Maldi TOF analysis of the purified final product gave an average mass of 62,842 Da. (DAR=7.4). Yield 75 mg, 50%.

### Synthesis of Int-321

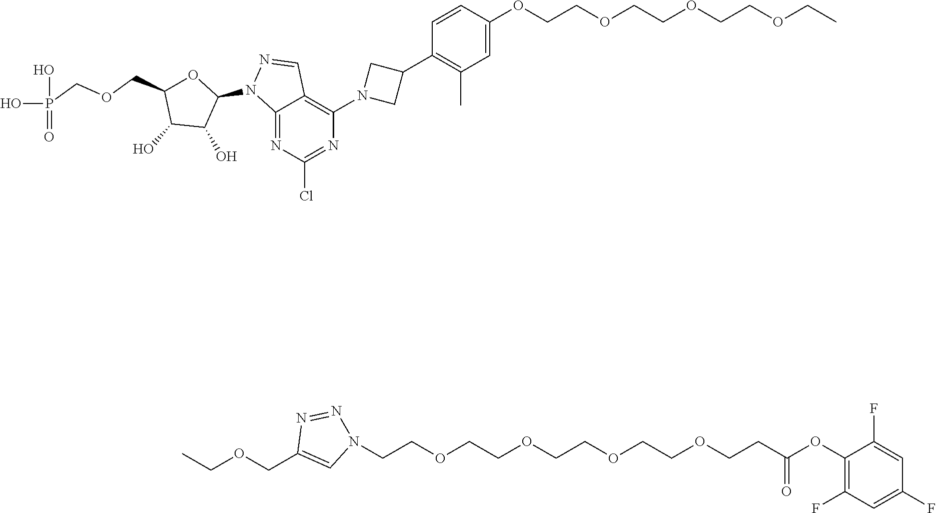

The title compound was prepared analogously to Int-298 where 4-(benzyloxy)phenylboronic acid was replaced with [4-(benzyloxy)-2-methylphenyl]boronic acid. The product was a white solid. Ion(s) found by LCMS [(M+2H)/2]⁺=589.4.

### Synthesis of Conjugate 188

Int-321 (25.6 mg, 0.0217 mmol) in DMF (0.5 mL) was added to SEQ ID NO: 80 (150 mg, 7.75 mL in PBS at pH 8.4 as described in the general conjugation procedure. Maldi TOF analysis of the purified final product gave an average mass of 61,723 Da. (DAR=6.3). Yield 80 mg, 54%.

### Synthesis of Int-322

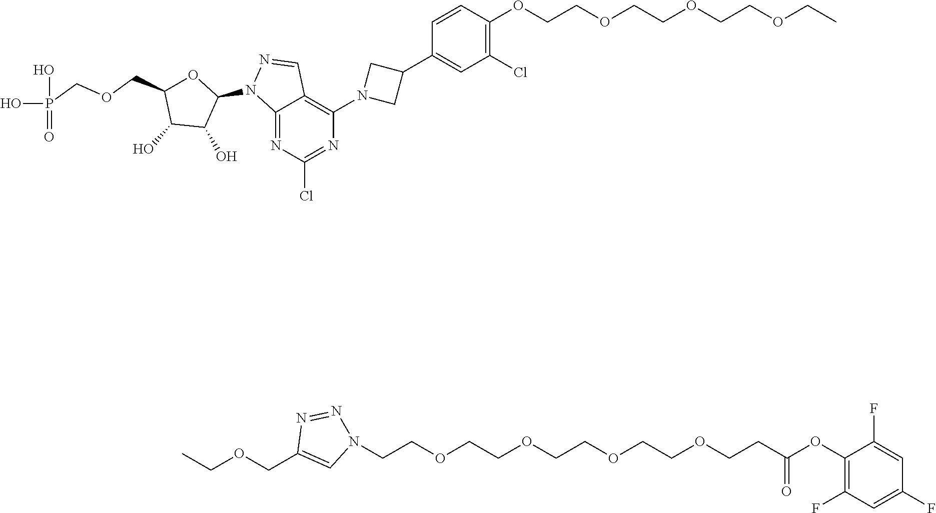

The title compound was prepared analogously to Int-298, where 4-(benzyloxy)phenylboronic acid was replaced with [4-(benzyloxy)-3-chlorophenyl]boronic acid. The product was a white solid. Ion(s) found by LCMS [(M+2H)/2]⁺=599.2 and 600.2.

### Synthesis of Conjugate 189

Int-322 (36 mg, 0.030 mmol) in DMF (0.5 mL) was added to SEQ ID NO: 80 (120 mg, 9.3 mL in PBS at pH 8.4) as described in the general conjugation procedure. Maldi TOF analysis of the purified final product gave an average mass of 63,782 Da. (DAR=8.1). Yield 64 mg, 53%.

### Synthesis of Int-300

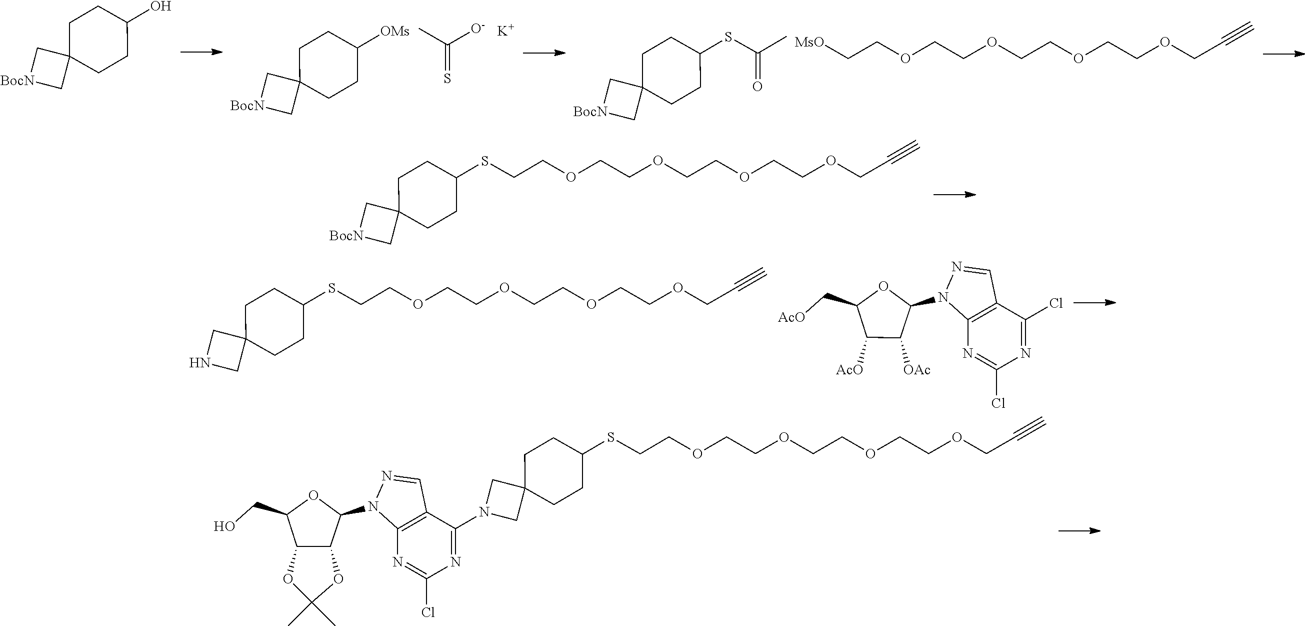

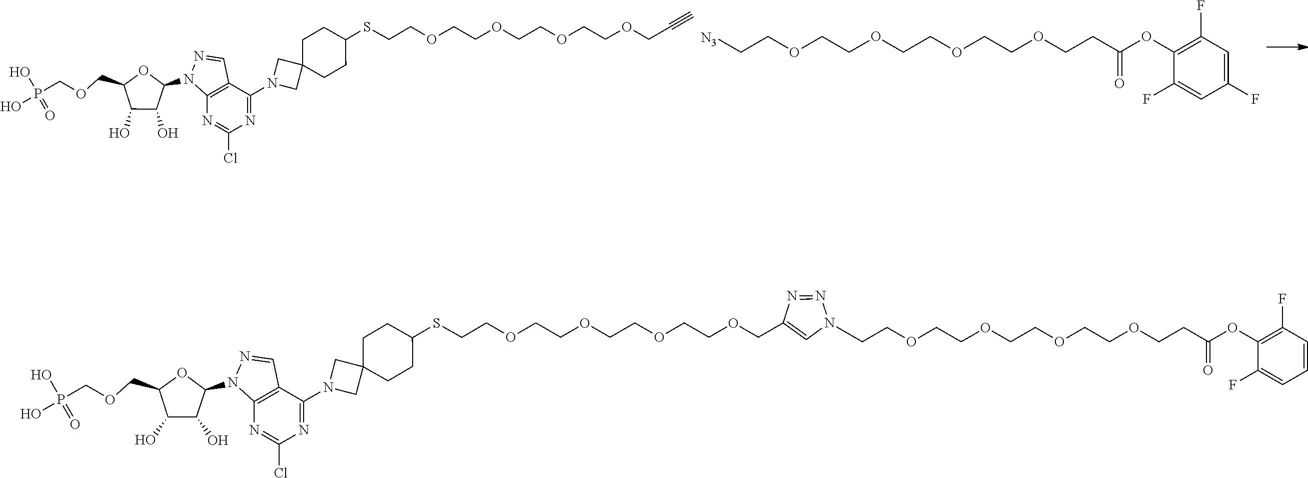

### Step a

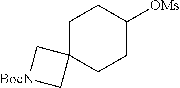 [SMILES: CC(C)(C)OC(=O)N1CC2(CCC(OS(C)(=O)=O)CC2)C1]

To a solution of alcohol (830 mg, 3.4 mmol) in DCM (10 ml) was added Et₃N (1.4 ml, 10 mmol), followed by methanesulfonyl chloride (0.32 ml, 4.1 mmol). The solution was stirred at room temperature for 2 hours, and then washed with HCl (0.5N, aq), dis H₂O and brine. The organic layer was separated and dried over sodium sulfate, filtered, and concentrated to yield product. The product was a white solid. Yield 910 mg, 83%. Ion(s) found by LCMS: [M+H]⁺=320.1.

### Step b

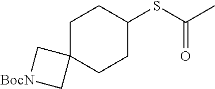 [SMILES: CC(=O)SC1CCC2(CC1)CN(C(=O)OC(C)(C)C)C2]

To a solution of the product from the previous step (728 mg, 2.2 mmol) in DMF (10 ml) was added potassium thioacetate (1.3 g, 11.4 mmol) and potassium carbonate (315 mg, 2.2 mmol). The solution was stirred at 70° C. for 3 days, cooled to room temperature, filtered and concentrated. The residue was purified by flash chromatography (DCM: 10% MeOH in DCM) to yield product Yield 400 mg, 58%. Ion(s) found by LCMS: [M+H]⁺=300.1.

### Step c

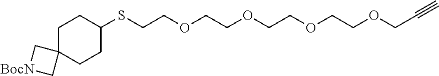 [SMILES: C#CCOCCOCCOCCOCCSC1CCC2(CC1)CN(C(=O)OC(C)(C)C)C2]

To a solution of step-b product (400 mg, 1.3 mmol) and propargyl-PEG4-mesyl ester (450 mg, 1.46 mmol) under nitrogen was added NaOMe (in MeOH, 3 ml, 1.46 mmol). The reaction mixture was stirred for 30 min, quenched with saturated ammonium chloride and extracted with EtOAc. The organic phase was washed with brine, dried over sodium sulfate and concentrated to give crude product, which was purified by semi-preparative HPLC (10-100% ACN: H₂O, 0.1% TFA modifier). Yield 410 mg, 65%. Ion(s) found by LCMS: [M+H]⁺=472.2.

### Step d

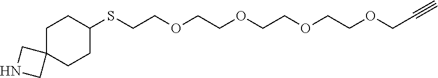 [SMILES: C#CCOCCOCCOCCOCCSC1CCC2(CC1)CNC2]

Intermediate N-Boc derivative from the previous step (410 mg) was dissolved in DCM (10 mL) and TFA (4 mL). The resulting mixture was stirred for 4 h at room temperature then solvent was removed under reduced pressure and dried under vacuum to yield the desired compound as its TFA salt. Yield 360 g, 100%. Ion(s) found by LCMS: [M+H]⁺=372.1.

### Step e

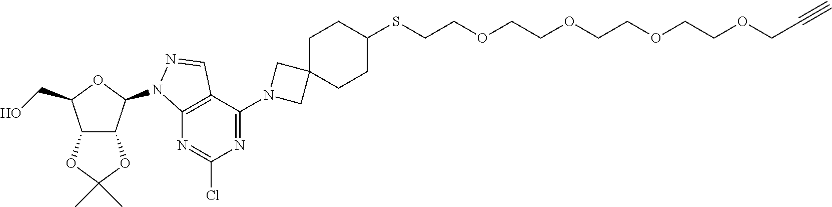 [SMILES: C#CCOCCOCCOCCOCCSC1CCC2(CC1)CN(c1nc(Cl)nc3c1cnn3[C@@H]1O[C@H](CO)[C@H]3OC(C)(C)O[C@H]31)C2]

A solution of intermediate b (520 mg, 1.1 mmol), step-d product (360 mg, 0.9 mmol) and triethylamine (0.2 mL, 1.45 mmol) in anhydrous EtOH (2 mL) was stirred at room temperature for 30 min. The solution was concentrated and the residue was dissolved in 7 M NH3/MeOH (5 mL). The reaction was stirred at room temperature overnight and mixture was concentrated to dryness under reduced pressure, then dissolved in acetone (1.5 mL). 2,2-dimethoxypropane (1.5 mL, 12 mmol) and p-TsOH (230 mg, 1.2 mmol) was added and the mixture was stirred overnight at room temperature for 2 hours. The reaction mixture was concentrated to dryness under reduced pressure and purified by flash chromatography (DCM: 10% MeOH in DCM) to yield product. The product was a white foam. Yield 360 mg, 53%. Ion(s) found by LCMS: [M+H]⁺=696.2.

### Step f

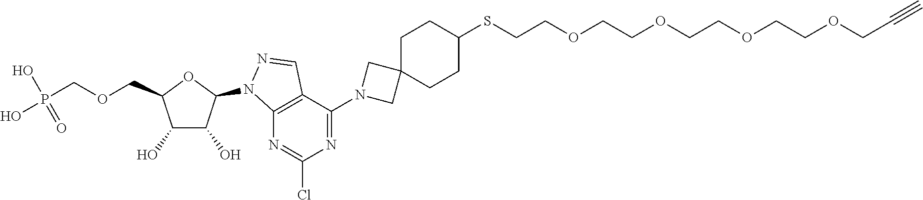 [SMILES: C#CCOCCOCCOCCOCCSC1CCC2(CC1)CN(c1nc(Cl)nc3c1cnn3[C@@H]1O[C@H](COCP(=O)(O)O)[C@@H](O)[C@H]1O)C2]

A solution of acetonide derivative from previous step (360 mg, 0.51 mmol) and [bis(tert-butoxy)carbonyl]methyl (trifluoromethyl)sulfonate (550 mg, 1.55 mmol) in dry DMF (5 mL) was cooled using ice-water bath. 60% NaH in mineral oil (125 mg, 3.1 mmol) was added to this and stirred at room temperature for 2 h. The reaction mixture was quenched with saturated ammonium chloride and extracted with EtOAc. The organic phase was washed with brine and dried over sodium sulfate concentrated to give crude product which was used in the next step without purification. Crude product was dissolved in 50% aqueous TFA (10 mL) and stirred at room temperature for 12 h, concentrated and the residue was purified by semi-preparative HPLC (10-100% ACN: H₂O, 0.1% TFA modifier). The product was a white solid. Yield 200 mg, 52%. Ion(s) found by LCMS: [M+H]⁺=750.1.

### Step g

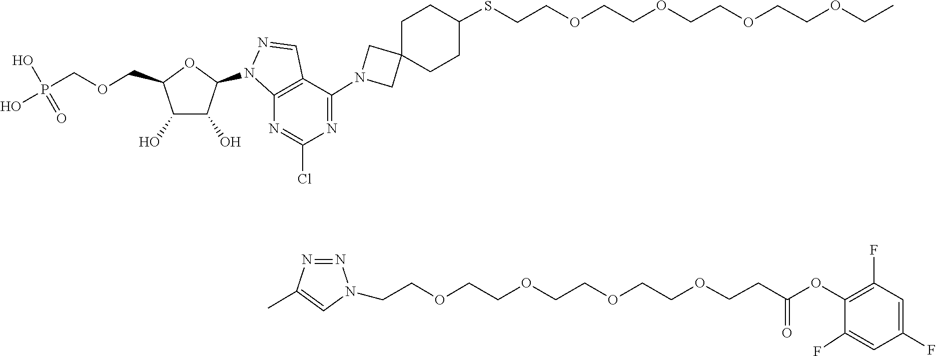

To a solution of the product from the previous step (90 mg, 0.12 mmol) and azido-PEG4-trifluorophenyl ester (65 mg, 0.155 mmol) dissolved in DMF (0.6 mL) was cooled to 0° C., and treated with a pre-mixed solution of a solution of copper(II) sulfate (0.005 mg, 0.03 mmol), sodium ascorbate (23 mg, 0.12 mmol), and THPTA (26 mg, 0.06 mmol) dissolved in water (0.6 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a white solid. Yield 110 mg, 78%. Ion(s) found by LCMS [(M+2H)/2]⁺=586.1.

### Synthesis of Conjugate 167

Int-300 (16 mg, 0.014 mmol) in DMF (0.5 mL) was added to SEQ ID NO: 80 (100 mg in 7.7 mL PBS 7.4, 0.0018 mmol) in PBS at pH 7.4 as described in the general conjugation procedure. Maldi TOF analysis of the purified final product gave an average mass of 61,103 Da. (DAR=5.7). Yield 62.4 mg, 62.4%.

### Synthesis of Int-301

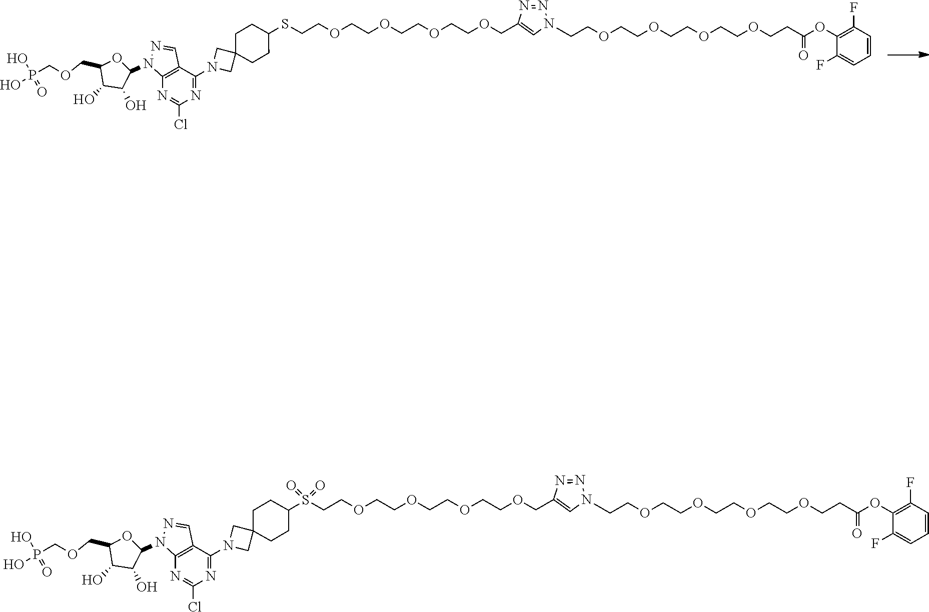

To a solution of Int-300 (30 mg, 0.025 mmol) in water and MeOH (1:1, 2 mL) was added oxone portion wise (63 mg, 0.0102 mmol). The reaction solution was stirred for 10 min and concentrated. The residue was purified by semi-preparative HPLC (10-100% ACN: H₂O, 0.1% TFA modifier). Yield 25 mg, 81%. Ion(s) found by LCMS [(M+2H)/2]⁺=602.5.

### Synthesis of Conjugate 168

Int-301 (8.7 mg, 0.007 mmol) in DMF (0.3 mL) was added to SEQ ID NO: 80 (50 mg in 3.8 mL PBS 7.4, 0.0009 mmol) as described in the general conjugation procedure. Maldi TOF analysis of the purified final product gave an average mass of 57,475 Da. (DAR=2.1). Yield 27.5 mg, 55.1%.

### Synthesis of Int-306

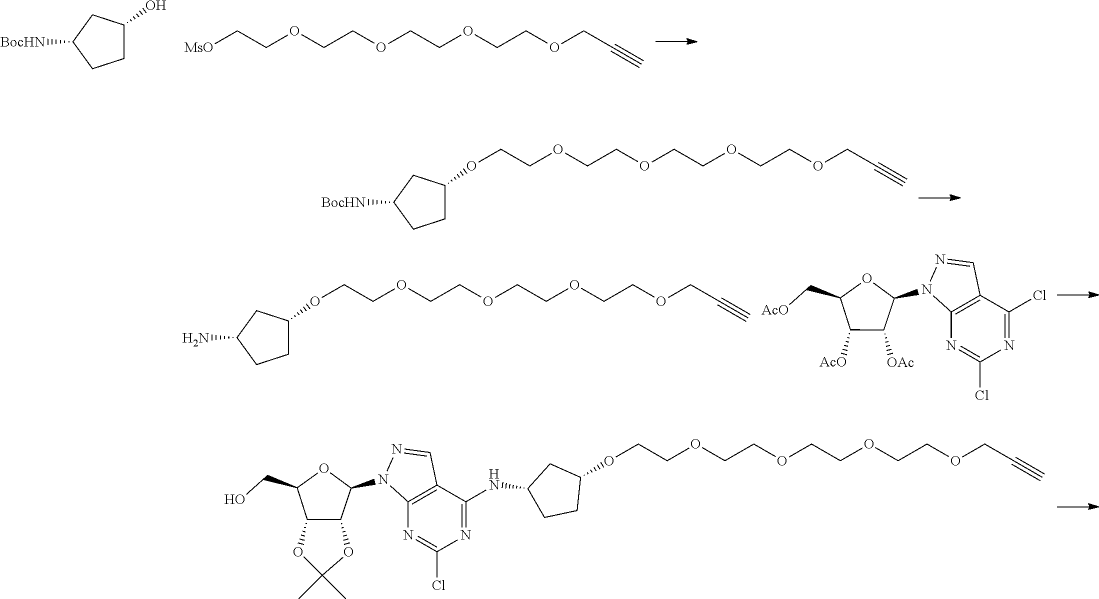

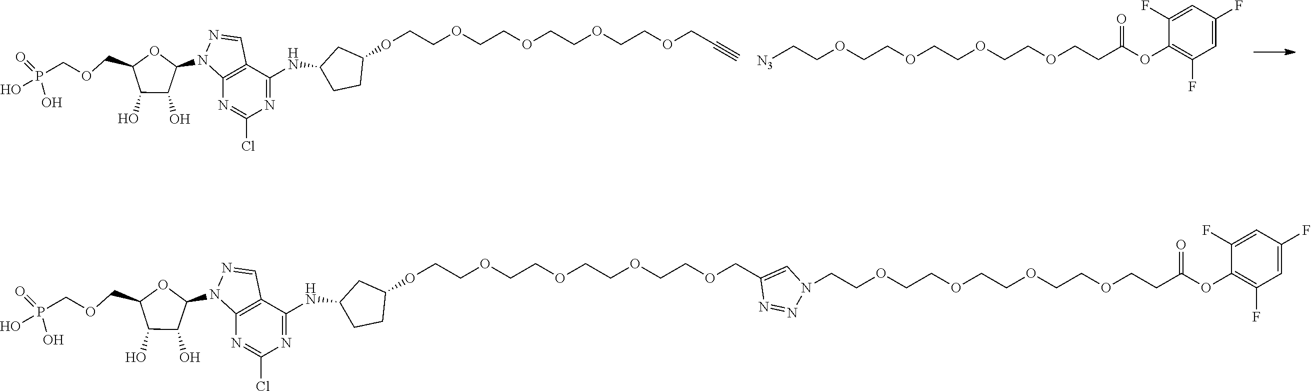

### Step a

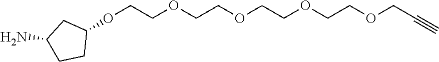 [SMILES: C#CCOCCOCCOCCOCCO[C@@H]1CC[C@H](N)C1]

To a solution of 3-aminocyclopentanol (1.0 g, 4.7 mmol) in DMF (10 mL) at 0° C. was added NaH (0.22 g, 5.6 mmol, 60% in mineral oil,) in portions, and the mixture was stirred for 1 h at room temperature. propargylPEG4-mesylate (1.6 g, 5.4 mmol) was added to it and stirred for 16 h at room temperature. The resulting solution was quenched with saturated ammonium chloride solution (10 mL) and extracted with EtOAc (3×20 mL). Combined organic extracts were washed with water, brine, dried over sodium sulfate, and concentrated to give the crude product which was further purified by semi-preparative HPLC (ACN: H₂O, O to 100%). Yield 1.1 g, 56%. Ion(s) found by LCMS: [M+H]⁺=416.2. Intermediate N-Boc derivative was dissolved in DCM (10 mL) and TFA (4 mL). The resulting mixture was stirred for 4 h at room temperature then solvent was removed under reduced pressure and dried under vacuum to yield the desired compound as its TFA salt. The product was a sticky oil. Yield 0.9 g, 100%. Ion(s) found by LCMS: [M+H]⁺=316.1.

### Step b

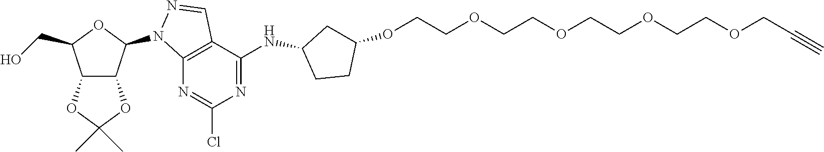 [SMILES: C#CCOCCOCCOCCOCCO[C@@H]1CC[C@H](Nc2nc(Cl)nc3c2cnn3[C@@H]2O[C@H](CO)[C@H]3OC(C)(C)O[C@H]32)C1]

A solution of triacetate intermediate B (400 mg, 0.9 mmol), step-a product (415 mg, 1 mmol) and triethylamine (0.2 mL, 1.45 mmol) in anhydrous EtOH (2 mL) was stirred at room temperature for 30 min. The solution was concentrated, and the residue was dissolved in 7 M NH₃/MeOH (4 mL). The reaction was stirred at room temperature overnight and mixture was concentrated to dryness under reduced pressure, then dissolved in acetone (1.5 mL). 2,2-dimethoxypropane (1.5 mL, 12 mmol) and p-TsOH (230 mg, 1.2 mmol) was added, and the mixture was stirred overnight at room temperature for 2 hours. The reaction mixture was concentrated to dryness under reduced pressure and purified by flash chromatography (DCM: 10% MeOH in DCM) to yield product. The product was a white foam. Yield 640 mg, 65%. Ion(s) found by LCMS: [M+H]⁺=640.2.

### Step c

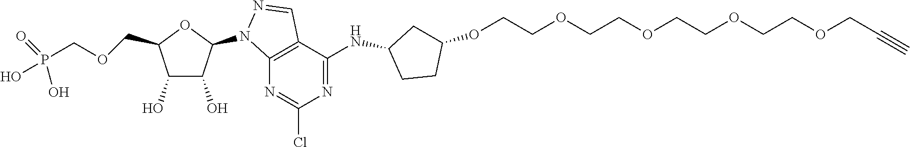 [SMILES: C#CCOCCOCCOCCOCCO[C@@H]1CC[C@H](Nc2nc(Cl)nc3c2cnn3[C@@H]2O[C@H](COCP(=O)(O)O)[C@@H](O)[C@H]2O)C1]

A solution of acetonide derivative from previous step (180 mg, 0.28 mmol) and [bis(tert-butoxy)carbonyl]methyl (trifluoromethyl)sulfonate (300 mg, 0.84 mmol) in dry DMF (mL) was cooled using ice-water bath. 60% NaH in mineral oil (67 mg, 1.68 mmol) was added to this and stirred at room temperature for 2 h. The reaction mixture was quenched with saturated ammonium chloride and extracted with EtOAc. The organic phase was washed with brine and dried over sodium sulfate concentrated to give crude product which was used in the next step without purification. Crude product was dissolved in 50% aqueous TFA (5 mL) and stirred at room temperature for 12 h, concentrated and the residue was purified by semi-preparative HPLC (10-100% ACN: H₂O, 0.1% TFA modifier). The product was a white solid. Yield 82 mg, 42%. Ion(s) found by LCMS: [M+H]⁺=694.2.

### Step d

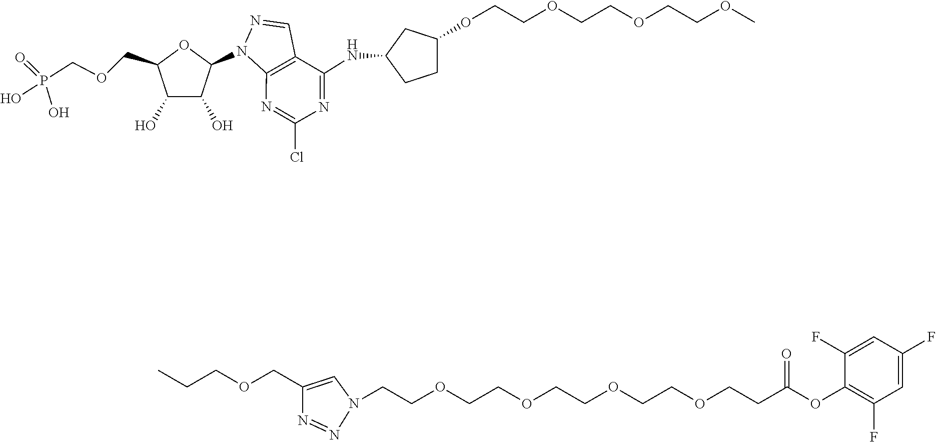

To a solution of the product from the previous step (43 mg, 0.061 mmol) and propargyl-PEG4-trifluorophenyl ester (33 mg, 0.08 mmol) in DMF (0.5 mL) was cooled to 0° C., and treated with a pre-mixed solution of a solution of copper(II) sulfate (2.4 mg, 0.015 mmol), sodium ascorbate (12 mg, 0.06 mmol), and THPTA (13 mg, 0.030 mmol) dissolved in water (0.5 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a white solid. Yield 43 mg, 62%. Ion(s) found by LCMS [(M+2H)/2]⁺=558.1.

### Synthesis of Conjugate 173

Int-306 (9 mg, 0.008 mmol) in DMF was added to SEQ ID NO: 80 (50 mg in 3.8 mL PBS 7.4, 0.0009 mmol) as described in the general conjugation procedure. Maldi TOF analysis of the purified final product gave an average mass of 60,669 Da. (DAR=5.6). Yield 27.2 mg, 54.3%.

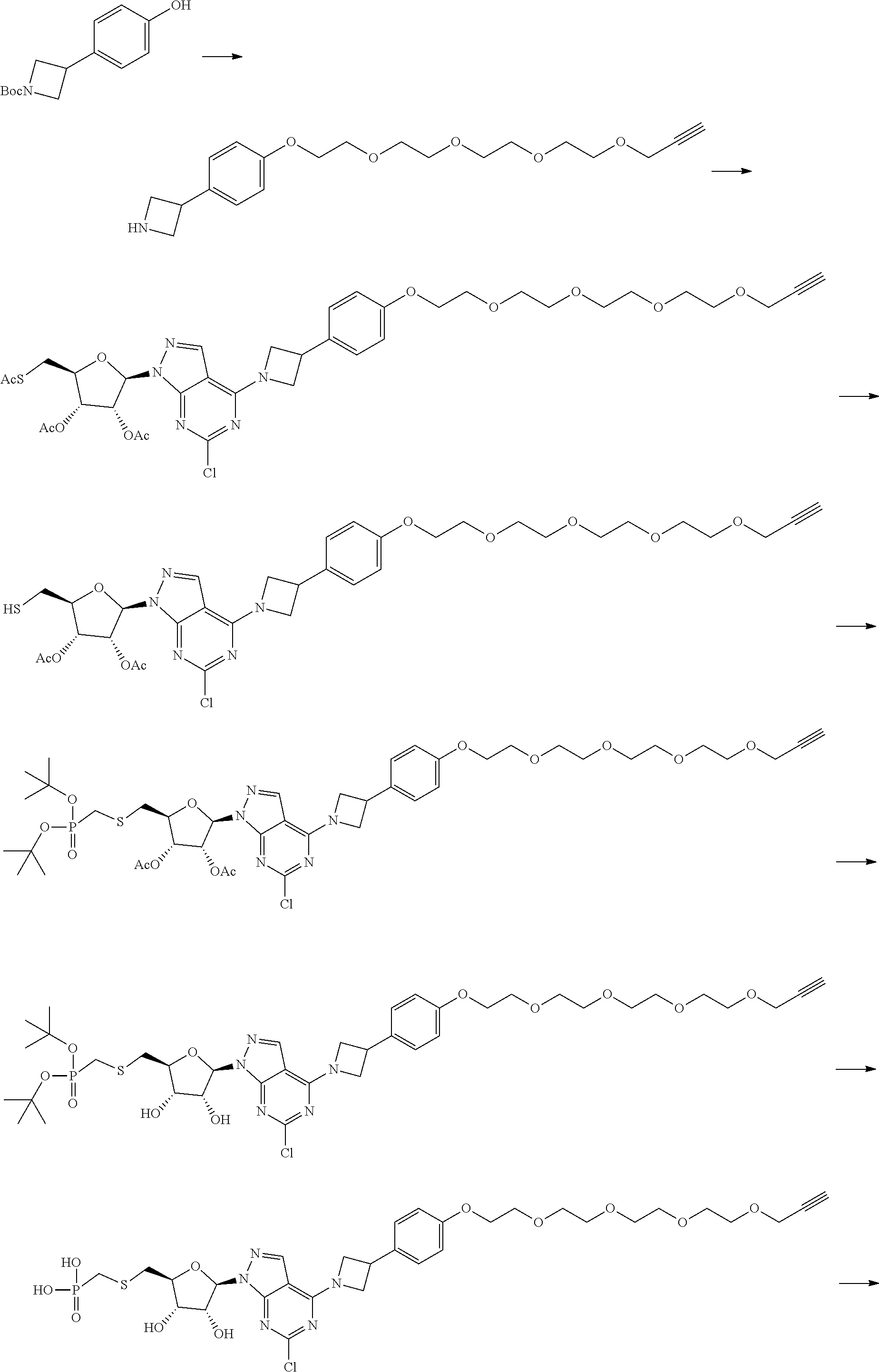

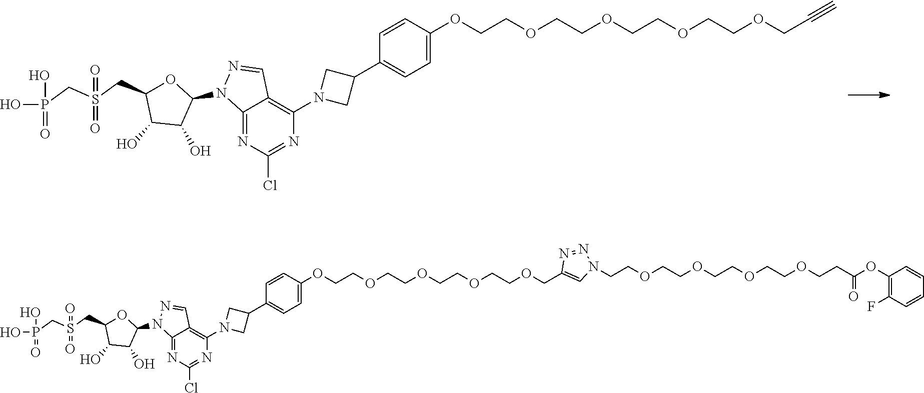 [SMILES: C#CCOCCOCCOCCOCCOc1ccc(C2CN(c3nc(Cl)nc4c3cnn4[C@@H]3O[C@H](CS(=O)(=O)CP(=O)(O)O)[C@@H](O)[C@H]3O)C2)cc1 ; O=C(CCOCCOCCOCCOCCn1cc(COCCOCCOCCOCCOc2ccc(C3CN(c4nc(Cl)nc5c4cnn5[C@@H]4O[C@H](CS(=O)(=O)CP(=O)(O)O)[C@@H](O)[C@H]4O)C3)cc2)nn1)Oc1ccccc1F]

### Synthesis of Int-307

### Step a

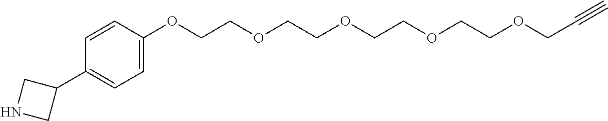 [SMILES: C#CCOCCOCCOCCOCCOc1ccc(C2CNC2)cc1]

Tert-Butyl 3-(4-hydroxyphenyl)azetidine-1-carboxylate (1 g, 4.0 mmol), propargyl-PEG4-mesylate (1.9 g, 6.0 mmol), and cesium carbonate (1.3 g, 4.0 mmol) were stirred in DMF (8 ml) at 80° C. for 4 hours. The mixture was diluted with DI water and extracted into ethyl acetate (25 ml×3). The combined organic layers were dried over sodium sulfate and concentrated. The crude residue was stirred in 4N HCl in dioxane (30 ml) at ambient temperature for 45 minutes. The mixture was concentrated and purified by silica gel chromatography (DCM/methanol) to afford the product as a light pink colored oil. Yield 1.1 g, 75%. Ion found by LCMS: [M+H]⁺=364.4.

### Step b

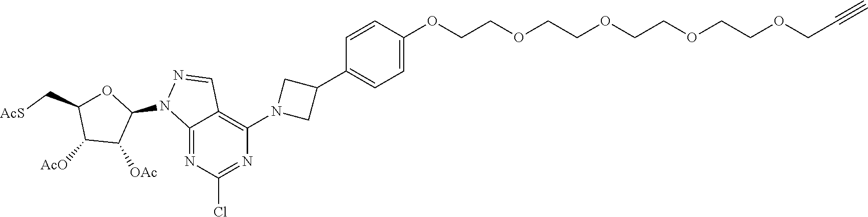

Product from step d as described in Int-286 (231.7 mg, 0.5 mmol) was dissolved in anhydrous DMF (1.0 ml) and added with product from step a (240 mg, 0.6 mmol) and DIPEA (1.5 mmol). The resulting mixture was stirred for 50 minutes, then directly purified through RPLC (100 g, 5 to 80% acetonitrile and water, using 0.1% TFA as modifier). Yield 219.3 mg, 55.4%. Ions found by LCMS: [M+Na]⁺=812.2, [M+H]⁺=790.2.

### Step c

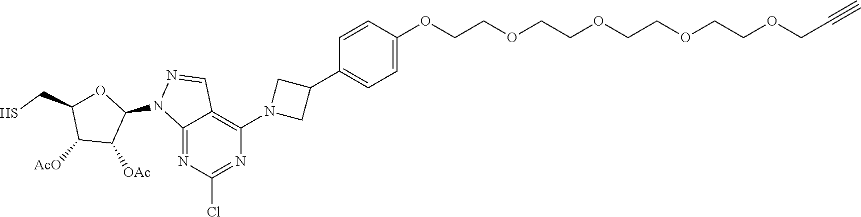 [SMILES: C#CCOCCOCCOCCOCCOc1ccc(C2CN(c3nc(Cl)nc4c3cnn4[C@@H]3O[C@H](CS)[C@@H](OC(C)=O)[C@H]3OC(C)=O)C2)cc1]

A solution of product from step b (219.3 mg, 0.277 mmol) in anhydrous DMF (1 ml) was bubbled with nitrogen, then added with DL-Dithiothreitol (60.3 mg, 0.39 mmol) and DIPEA (143.5 mg, 1.1 mmol). The resulting mixture was stirred under nitrogen for 1 hour. It was cooled to room temperature and carried to subsequent step without purification. Ions found by LCMS: [M+Na]⁺=770.2, [M+H]⁺=748.2.

### Step d

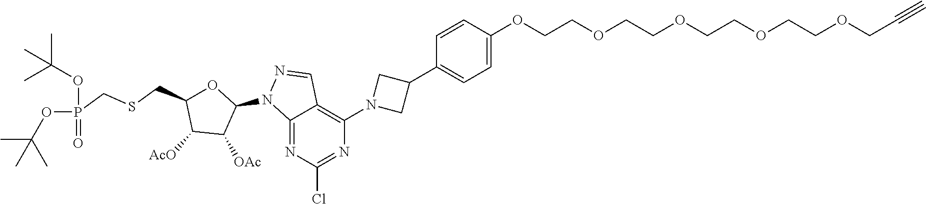 [SMILES: C#CCOCCOCCOCCOCCOc1ccc(C2CN(c3nc(Cl)nc4c3cnn4[C@@H]3O[C@H](CSCP(=O)(OC(C)(C)C)OC(C)(C)C)[C@@H](OC(C)=O)[C@H]3OC(C)=O)C2)cc1]

The reaction mixture from step c was added with methanesulfonic acid, 1,1,1-trifluoro-, [bis(1,1-dimethylethoxy)phosphinyl]methyl ester (197.4 mg, 0.554 mmol), followed by potassium iodide (92 mg, 0.554 mmol). It was then heated at 60° C. under nitrogen overnight. After cooled to room temperature, the reaction mixture was carried to subsequent step without purification. Ions found by LCMS: [M−2tBu]⁺=842.2, [(M−2tBu)/2]⁺=421.6.

### Step e

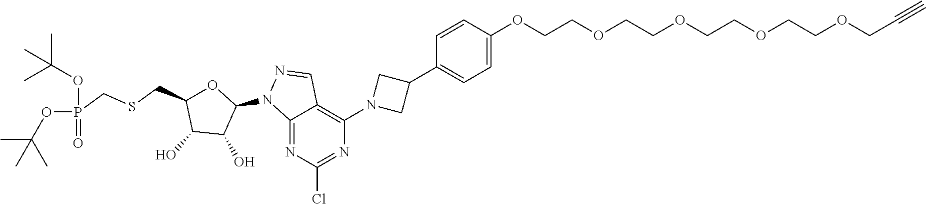 [SMILES: C#CCOCCOCCOCCOCCOc1ccc(C2CN(c3nc(Cl)nc4c3cnn4[C@@H]3O[C@H](CSCP(=O)(OC(C)(C)C)OC(C)(C)C)[C@@H](O)[C@H]3O)C2)cc1]

To a solution of step b reaction was added K₂CO₃ (414.6 mg, 3 mmol) and MeOH (5 ml). The resulting mixture was stirred for 3 hours. The salt was filtered off and washed with acetonitrile. The filtrate was added with DOWEX 50 W×8 hydrogen form (1 g). After stirring for 5 minutes, the solid was filtered off, and the filtrate was concentrated by rotary evaporation. The residue was carried to subsequent step without purification. Ions found by LCMS: [M−2tBu]⁺=758.2, [(M−2tBu)/2]⁺=379.6.

### Step f

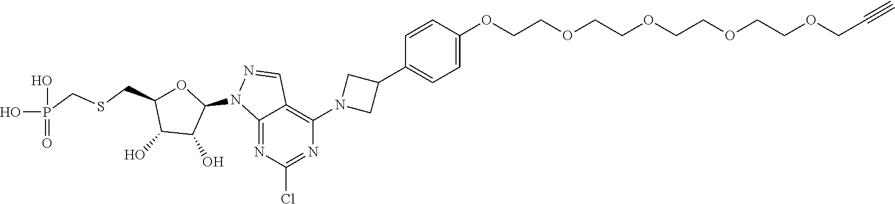 [SMILES: C#CCOCCOCCOCCOCCOc1ccc(C2CN(c3nc(Cl)nc4c3cnn4[C@@H]3O[C@H](CSCP(=O)(O)O)[C@@H](O)[C@H]3O)C2)cc1]

A crude product from step c was added with m. After the resulting mixture was stirred at room temperature for 2 hours, it was concentrated and purified through RPLC (100 g, 5 to 80% acetonitrile and water). Yield 101.4 mg, 48.3% over four steps. Ions found by LCMS: [M+Na]⁺=780.2, [M+H]⁺=758.2.

### Step g

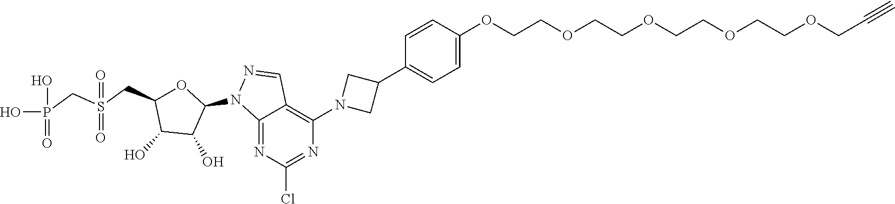 [SMILES: C#CCOCCOCCOCCOCCOc1ccc(C2CN(c3nc(Cl)nc4c3cnn4[C@@H]3O[C@H](CS(=O)(=O)CP(=O)(O)O)[C@@H](O)[C@H]3O)C2)cc1]

To a solution of product from step f (101 mg, 0.133 mmol) in MeOH (2 ml) was added Oxone (328 mg, 0.533 mmol) and water (0.2 ml). The resulting mixture was stirred overnight, the solid was filtered off and was washed with acetonitrile. The filtrate was concentrated and purified through RPLC (100 g, 5 to 100% acetonitrile and water). Yield 38.2 mg, 36.3%. Ions found by LCMS: [M+Na]⁺=812.2, [M+H]⁺=790.2.

### Step h

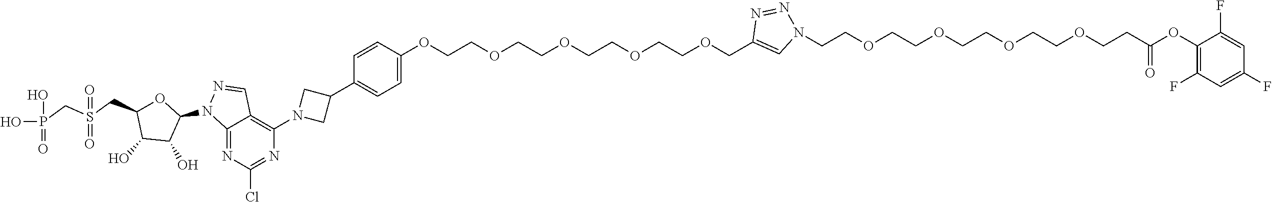 [SMILES: O=C(CCOCCOCCOCCOCCn1cc(COCCOCCOCCOCCOc2ccc(C3CN(c4nc(Cl)nc5c4cnn5[C@@H]4O[C@H](CS(=O)(=O)CP(=O)(O)O)[C@@H](O)[C@H]4O)C3)cc2)nn1)Oc1c(F)cc(F)cc1F]

To a solution of propargyl-PEG4-trifluorophenyl ester (14.7 mg, 0.0349 mmol) in anhydrous DMF (0.4 ml) was added a premixed mixture of THPTA (15.8 mg, 0.0349 mmol), Cu₂SO₄ (5.5 mg, 0.0349 mmol) in water (0.2 ml). After stirring for 5 minutes, the mixture was added into product from step g (23 mg, 0.0291 mmol), followed by MeOH (0.2 ml) and sodium ascorbate (19.8 mg, 0.1 mmol). The reaction was stirred for one hour, then added with TFA (0.2 ml) and directly purified by HPLC (5 to 70% acetonitrile and water, using 0.1% TFA as modifier). Yield 19.1 mg, 54.2%. Ions found by LCMS: [(M+2H)/2]+=606.2.

### Synthesis of Conjugate 174

To a solution of SEQ ID NO: 80 (5.23 ml, 67.5 mg, 0.001222 mmol) in PBS 7.4 was added Int-307 (11.1 mg, 0.009162 mmol) in DMF (1.3 ml). Addition of DMF (0.2 ml×5) was used to wash the glassware and combined to the reaction mixture. The pH of the reaction mixture was slowly adjusted to 8.5 by 20× borate buffer solution (0.2 ml) and purified by dialysis and SEC. Maldi TOF analysis of the purified final product gave an average mass of 60365 Da (DAR=4.8). Yield: 38.4 mg, 56.9%.

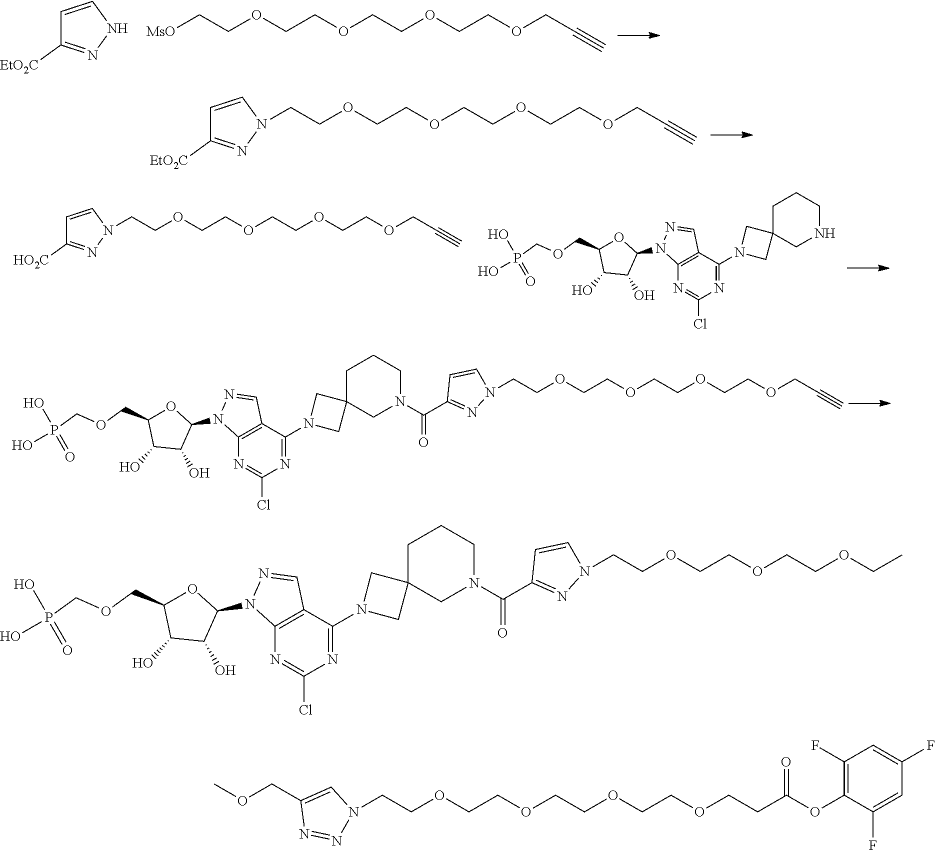

### Synthesis of Int-308

### Step a

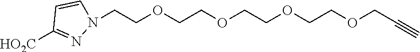 [SMILES: C#CCOCCOCCOCCOCCn1ccc(C(=O)O)n1]

To a solution of ethyl 2H-pyrazole-3-carboxylate (0.7 g, 5 mmol) and propargyl-PEG4-mesylate (1.8 g, 6 mmol) in DMF (10 ml) was added K₂CO₃ (1.3 g, 10 mmol). The reaction mixture was heated at 70° C. for 1.5 days. After cooling to room temperature, the salt was filtered off and washed with acetonitrile. The filtrate was concentrated, and the residue was dissolved in MeOH (5 ml) and treated with a solution of LiOH monohydrate (0.47 g, 20 mmol) in water (5 ml). The mixture was stirred at room temperature for 2 hours, then acidified with TFA and residue was purified by semi-preparative HPLC (5% to 100% ACN/water, 0.1% TFA modifier) to give the product as a solid. Yield 0.52 g, 32%. Ion(s) found by LCMS: [M+H]⁺=327.1.

### Step b

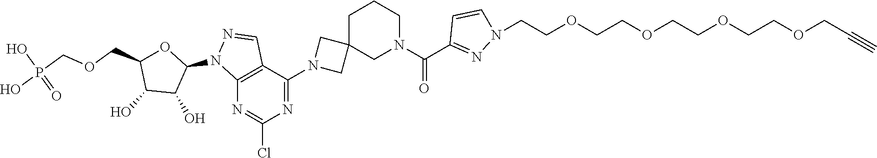 [SMILES: C#CCOCCOCCOCCOCCn1ccc(C(=O)N2CCCC3(C2)CN(c2nc(Cl)nc4c2cnn4[C@@H]2O[C@H](COCP(=O)(O)O)[C@@H](O)[C@H]2O)C3)n1]

To a solution of spirocyclic piperidine (800 mg, 1.6 mmol) described in Synthesis of Int-287 and pyrazole-acid (0.52 mg, 1.6 mmol) in DMF was added DIEA (0.83 mL, 4.8 mmol) and HATU (1.8 mg, 4.8 mmol). The reaction solution was stirred at room temperature for 2 hours. The reaction was concentrated, and the residue was purified by semi-preparative HPLC (5% to 100% ACN/water, 0.1% TFA modifier) to give the product as an off-white foam. Yield 920 mg, 70%. Ion(s) found by LCMS: [M+H]⁺=813.2.

### Step c

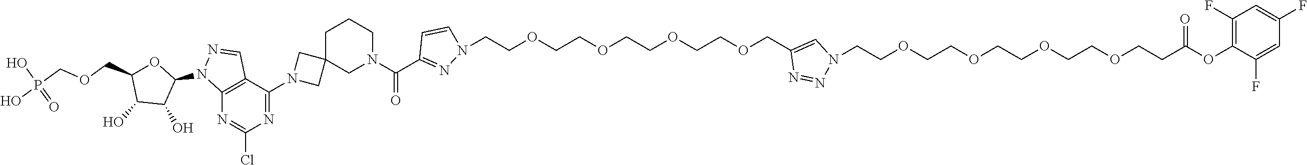 [SMILES: O=C(CCOCCOCCOCCOCCn1cc(COCCOCCOCCOCCn2ccc(C(=O)N3CCCC4(C3)CN(c3nc(Cl)nc5c3cnn5[C@@H]3O[C@H](COCP(=O)(O)O)[C@@H](O)[C@H]3O)C4)n2)nn1)Oc1c(F)cc(F)cc1F]

To a solution of the product from the previous step (0.65 g, 0.8 mmol) and propargyl-PEG4-trifluorophenyl ester (0.43 g, 1 mmol) in DMF (4 mL) cooled to 0° C., and treated with a pre-mixed solution of a solution of copper(II) sulfate (32 mg, 0.2 mmol), sodium ascorbate (0.31 g, 1.6 mmol), and THPTA (0.17 mg, 0.4 mmol) dissolved in water (4 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a sticky oil. Yield 520 mg, 52%. Ion(s) found by LCMS [(M+2H)/2]⁺=617.7.

### Synthesis of Conjugate 175

Int-308 (90 mg, 0.072 mmol) in DMF (3 mL) was added to SEQ ID NO: 80 (423 mg in 32.3 mL PBS 7.4, 0.0076 mmol) in PBS at pH 7.4 as described in the general conjugation procedure. Maldi TOF analysis of the purified final product gave an average mass of 60,394 Da. (DAR=4.7). Yield 316.2 mg, 73.5%.

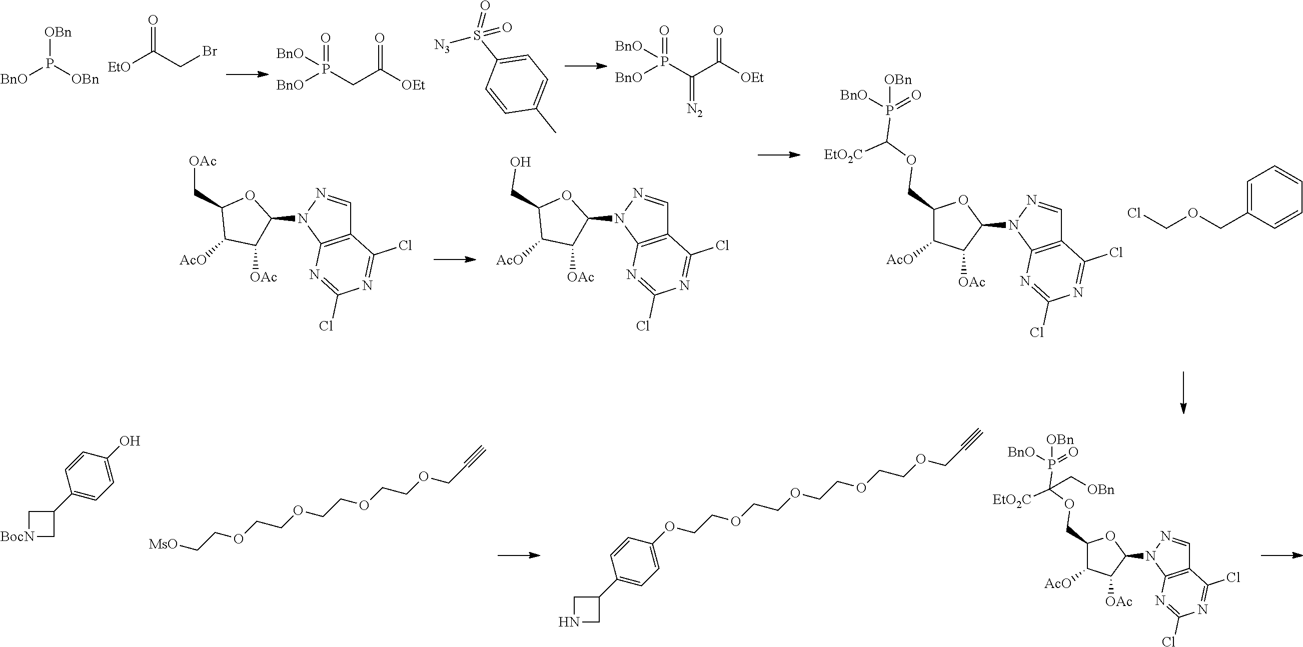

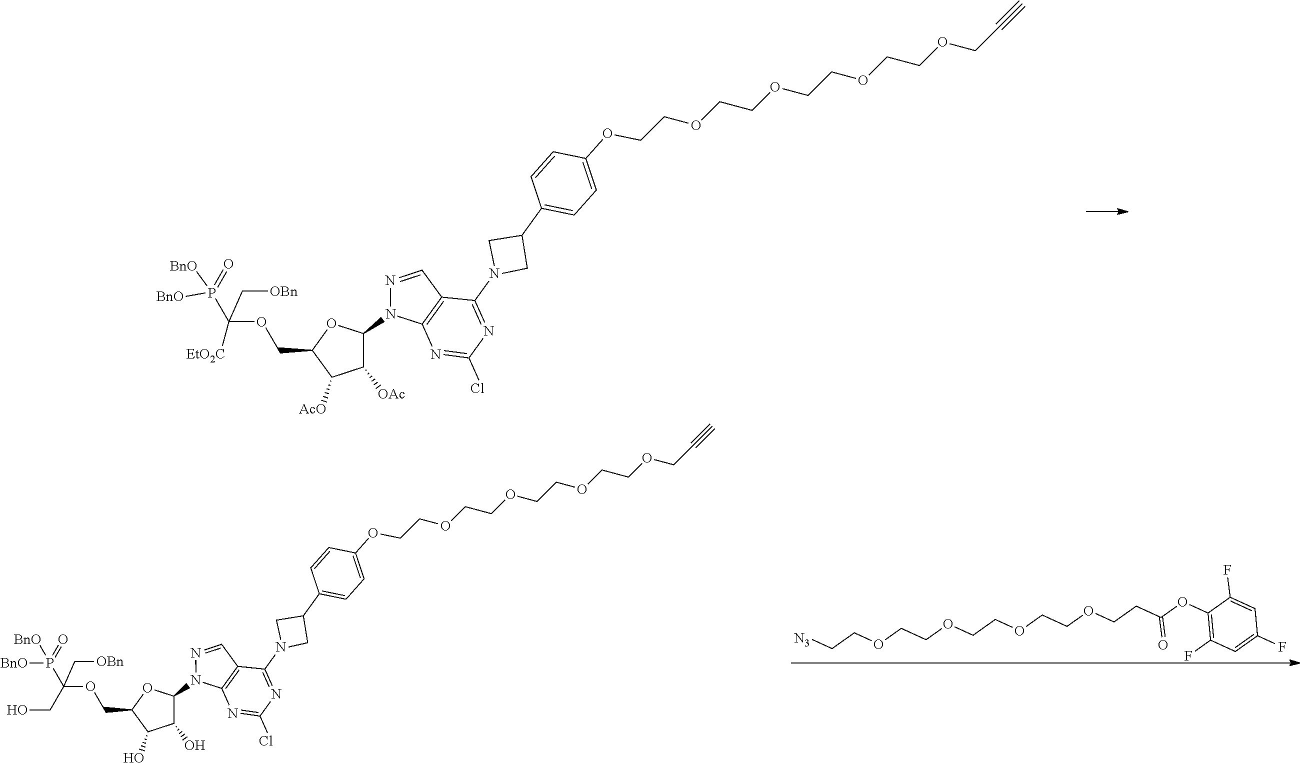 [SMILES: C#CCOCCOCCOCCOCCOc1ccc(C2CN(c3nc(Cl)nc4c3cnn4[C@@H]3O[C@H](COC(COCc4ccccc4)(C(=O)OCC)P(=O)(OCc4ccccc4)OCc4ccccc4)[C@@H](OC(C)=O)[C@H]3OC(C)=O)C2)cc1 ; C#CCOCCOCCOCCOCCOc1ccc(C2CN(c3nc(Cl)nc4c3cnn4[C@@H]3O[C@H](COC(CO)(COCc4ccccc4)P(=O)(OCc4ccccc4)OCc4ccccc4)[C@@H](O)[C@H]3O)C2)cc1 ; [N-]=[N+]=NCCOCCOCCOCCOCCC(=O)Oc1c(F)cc(F)cc1F]

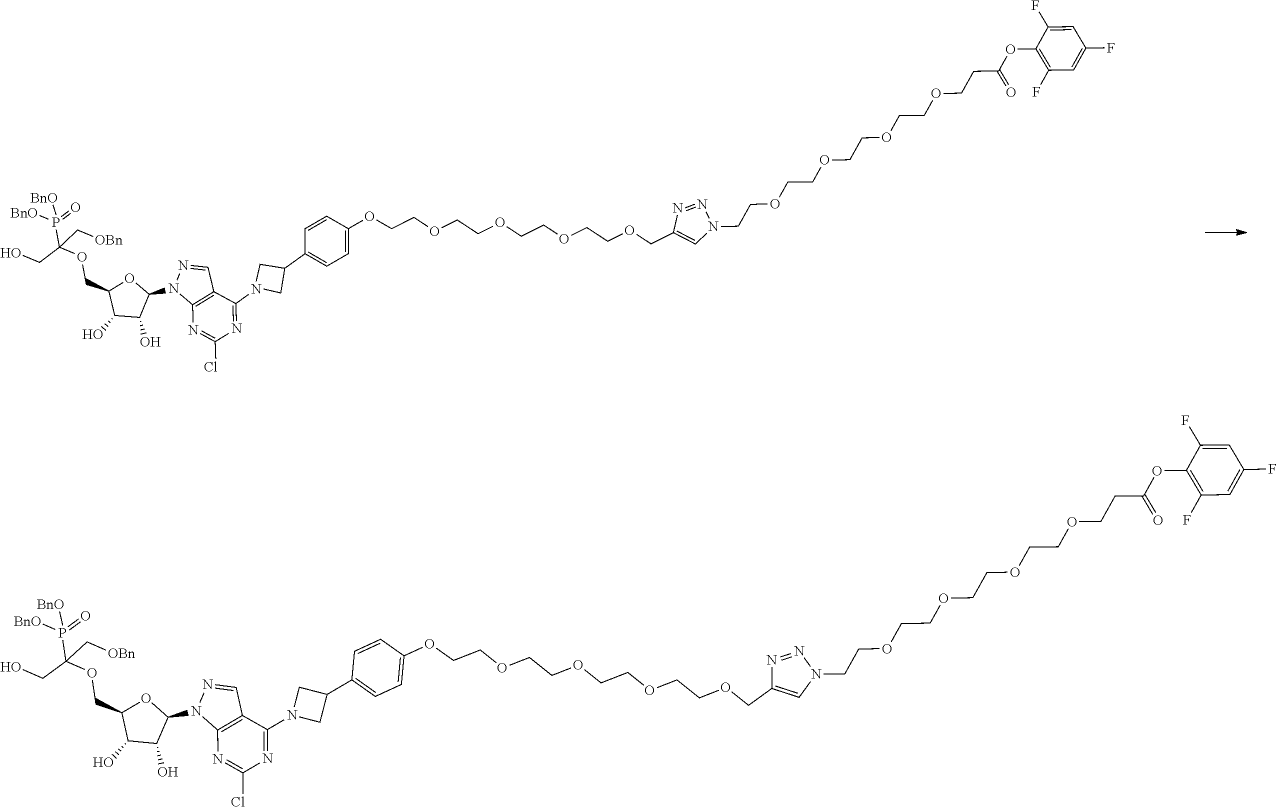 [SMILES: O=C(CCOCCOCCOCCOCCn1cc(COCCOCCOCCOCCOc2ccc(C3CN(c4nc(Cl)nc5c4cnn5[C@@H]4O[C@H](COC(CO)(COCc5ccccc5)P(=O)(OCc5ccccc5)OCc5ccccc5)[C@@H](O)[C@H]4O)C3)cc2)nn1)Oc1c(F)cc(F)cc1F ; O=C(CCOCCOCCOCCOCCn1cc(COCCOCCOCCOCCOc2ccc(C3CN(c4nc(Cl)nc5c4cnn5[C@@H]4O[C@H](COC(CO)(COCc5ccccc5)P(=O)(OCc5ccccc5)OCc5ccccc5)[C@@H](O)[C@H]4O)C3)cc2)nn1)Oc1c(F)cc(F)cc1F]

### Synthesis of Int-309

### Step a

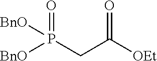 [SMILES: CCOC(=O)CP(=O)(OCc1ccccc1)OCc1ccccc1]

Bromo ethyl acetate (11.3 g, 67.9 mmol) was added to tribenzyl phosphite (10 g, 27.1 mmol) and the mixture was stirred for 4 hours at 100° C. The mixture was cooled to room temperature and purified by silica gel chromatography (ethyl acetate/hexanes) to afford the product as a clear viscous oil. Yield 6.3 g, 66%. Ion(s) found by LCMS: [M+H]⁺=349.2.

### Step b

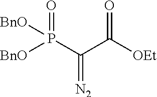

Tosyl azide (5.7 g, 28.7 mmol, in 50 mL DCM) was added dropwise, via an add funnel over a 2-hour period, to a cooled (0° C. ice bath) mixture of sodium tert butoxide and dibenzyl-ethyl-phosphono acetate (1 Og, 28.7 mmol in 100 mL of DCM). The mixture was stirred overnight allowing the ice bath to gradually rise to ambient temperature. The mixture filtered through celite and concentrated. The crude residue was purified by silica gel chromatography (hexanes/ethyl acetate). Yield 7.2 g, 67%. Ion(s) found by LCMS: [M+H]⁺=375.2.

### Step c

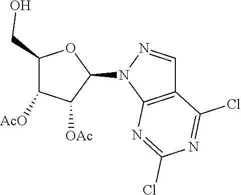 [SMILES: CC(=O)O[C@@H]1[C@H](OC(C)=O)[C@@H](CO)O[C@H]1n1ncc2c(Cl)nc(Cl)nc21]

The triacetate intermediate B (7 g, 15.7 mmol) was dissolved in MTBE (100 mL), lipase Candida rugosa (4.5 g) was added followed by 200 mL of phosphate buffer 0.2 M (pH 7.4). The reaction was stirred at 40° C. for 16 hours at which time the mixture was filtered through a plug of celite. The mixture was extracted into ethyl acetate and the combined organics were dried over sodium sulfate, concentrated, and purified by silica gel chromatography (ethyl acetate/hexanes). Yield 5.5 g, 86%. Ion(s) found by LCMS: [M+H]⁺=405.2.

### Step d

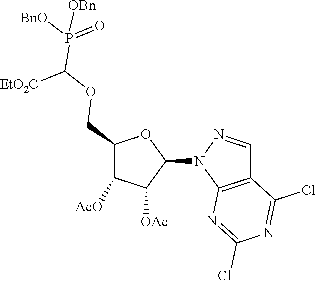 [SMILES: CCOC(=O)C(OC[C@H]1O[C@@H](n2ncc3c(Cl)nc(Cl)nc32)[C@H](OC(C)=O)[C@@H]1OC(C)=O)P(=O)(OCc1ccccc1)OCc1ccccc1]

The alcohol described in the previous step (2.6 g, 6.4 mmol) was dissolved in toluene (100 mL) in a sealed tube. The diazo intermediate described in step b of this example (4.8 g, 12.8 mmol) was added and the mixture was evacuated by vacuum flushed with nitrogen (3×). Rhodium tetra acetate (200 mg) was added, and the mixture was again evacuated and flushed with nitrogen (3×). The tube was sealed and placed in an oil bath and stirred at 110° C. for 16 hours. The mixture was cooled and filtered through a plug of celite. The solvent was removed by the rotary evaporator and the crude residue was purified by silica gel chromatography (hexanes/ethyl acetate). Yield 2.7 g, 56%. Ion(s) found by LCMS: [M+H]⁺=751.2.

### Step e

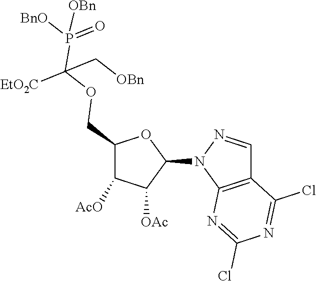 [SMILES: CCOC(=O)C(COCc1ccccc1)(OC[C@H]1O[C@@H](n2ncc3c(Cl)nc(Cl)nc32)[C@H](OC(C)=O)[C@@H]1OC(C)=O)P(=O)(OCc1ccccc1)OCc1ccccc1]

The ester described in the previous step (710 mg, 0.94 mmol) was dissolved in THE and cooled to −15° C. under an atmosphere of nitrogen. Sodium HMDS (1.2 mL, 1.23 mmol, 1M in THF) was added and the mixture was stirred for 10 minutes at −15° C. TBAI (349 mg, 0.94 mmol) was added followed by BOM chloride (296 mg, 1.89 mmol) and the reaction was stirred for 20 minutes. The reaction was quenched at −15° C. with saturated aqueous ammonium chloride, extracted into ethyl acetate (3×), and the combined organic extracts were dried over sodium sulfate and the solvent removed on the rotary evaporator. The crude residue was purified by silica gel chromatography (hexanes/ethyl acetate to afford the product as a clear oil. Yield 440 mg, 53%. Ion(s) found by LCMS: [M+H]⁺=871.2.

### Step f

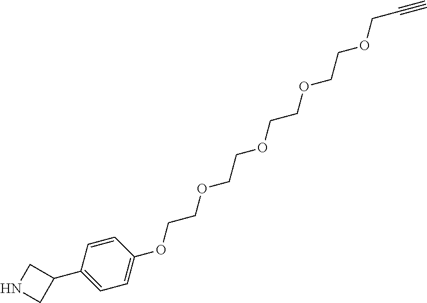 [SMILES: C#CCOCCOCCOCCOCCOc1ccc(C2CNC2)cc1]

tert-Butyl 3-(4-hydroxyphenyl)azolidine-1-carboxylate (1 g, 4.0 mmol), propargyl-peg₄-mesylate (1.9 g, 6.0 mmol), and cesium carbonate (1.3 g, 4.0 mmol) were stirred in DMF (8 mL) at 80° C. for 4 hours. The mixture was diluted with DI water and extracted into ethyl acetate (3×, 25 rnL). The combined organic extracts were dried over sodium sulfate and concentrated. The crude residue was stirred in 4N HCl in dioxane (30 mL) at ambient temperature for 45 minutes. The mixture was concentrated and purified by silica gel chromatography (dcm/methanol) to afford the product as a light pink colored oil. Yield 1.1 grams, 75%. Ions found by LCMS: [M+H]⁺=364.4

### Step g

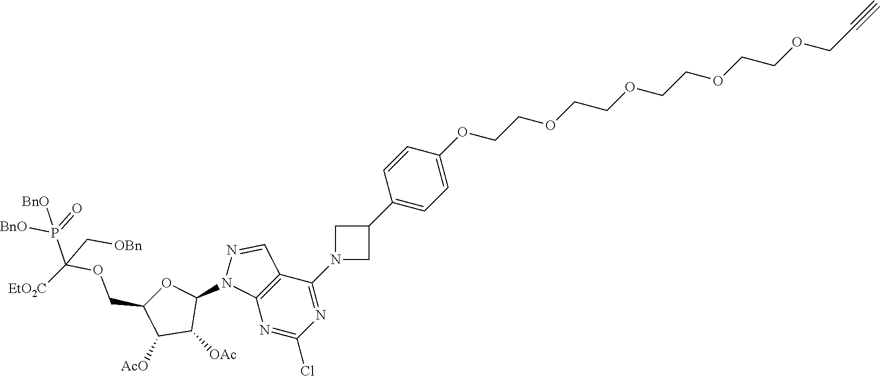 [SMILES: C#CCOCCOCCOCCOCCOc1ccc(C2CN(c3nc(Cl)nc4c3cnn4[C@@H]3O[C@H](COC(COCc4ccccc4)(C(=O)OCC)P(=O)(OCc4ccccc4)OCc4ccccc4)[C@@H](OC(C)=O)[C@H]3OC(C)=O)C2)cc1]

The protected ribose from step e (300 mg, 0.34 mmol), the azetidine from step f (162 mg, 0.45 mmol), and triethylamine (45 mg, 0.45 mmol) were stirred in ethanol (15 mL) at 50° C. for 2 hours. The solvent was removed by the rotary evaporator and the crude residue was purified by silica gel chromatography (hexanes/ethyl acetate) to afford the product as a clear oil. Yield 330 mg, 80%. Ion(s) found by LCMS: [M+H]⁺=1198.4.

### Step h

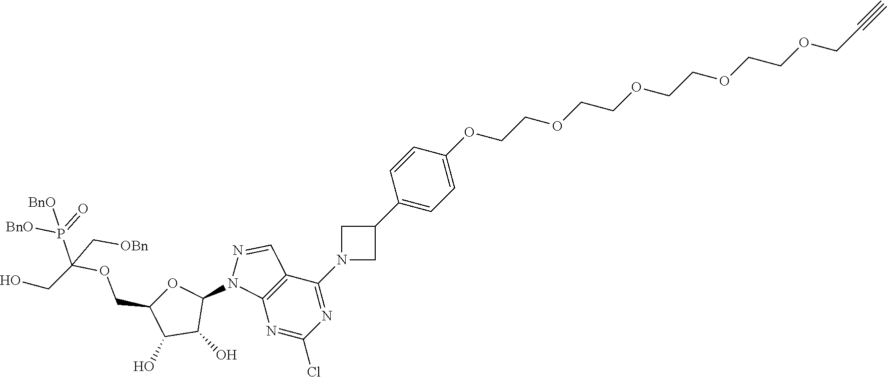 [SMILES: C#CCOCCOCCOCCOCCOc1ccc(C2CN(c3nc(Cl)nc4c3cnn4[C@@H]3O[C@H](COC(CO)(COCc4ccccc4)P(=O)(OCc4ccccc4)OCc4ccccc4)[C@@H](O)[C@H]3O)C2)cc1]

The ester from step g of this example was dissolved in ethanol (50 mL) and calcium chloride (83 mg, 0.75 mmol) was added followed by sodium borohydride (30 mg, 0.75 mmol) and the mixture was stirred for 2 hours at ambient temperature. The mixture was filtered through a plug of celite, washed with several small portions of ethanol. The solvent was reduced on the rotary evaporator and the residue was diluted with DI water (50 ml) and extracted into ethyl acetate (3×) taken up in ethyl acetate (3×, 20 mL). The combined organics were dried over sodium sulfate and concentrated on the rotary evaporator. The crude residue was purified by silica gel chromatography (hexanes/ethyl acetate) to afford the product as a clear oil. Yield 210 mg, 78%. Ion(s) found by LCMS: [M+H]⁺=1072.4.

### Step i

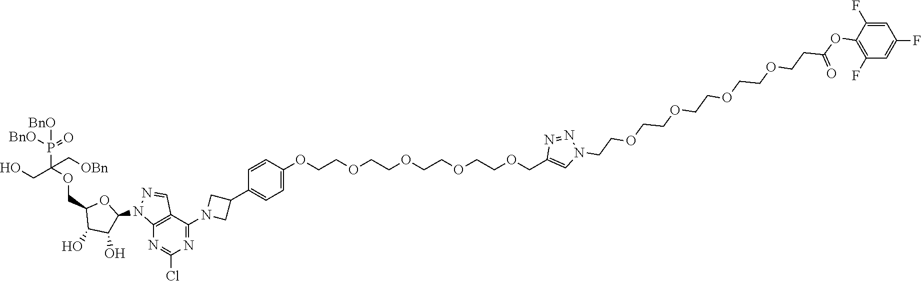 [SMILES: O=C(CCOCCOCCOCCOCCn1cc(COCCOCCOCCOCCOc2ccc(C3CN(c4nc(Cl)nc5c4cnn5[C@@H]4O[C@H](COC(CO)(COCc5ccccc5)P(=O)(OCc5ccccc5)OCc5ccccc5)[C@@H](O)[C@H]4O)C3)cc2)nn1)Oc1c(F)cc(F)cc1F]

The product from step h (160 mg, 0.15 mmol) and intermediate A (63 mg, 0.15 mmol) were dissolved in DMF (3 mL) and cooled to 0° C. In a separate vial copper sulfate (3 mg, 0.022 mmol, in 177 μL of water was added to a mixture of sodium ascorbate (89 mg, 0.45 mmol) and BTTA (17 mg, 0.037 mmol) in 1 mL of DI water and the mixture was shaken gently for 20 seconds at which point it was added in one portion to the stirring mixture of the acetate and azido-peg₄-trifluorophenol ester. The reaction was stirred at 0 C for 5 minutes and then allowed to warm to room temperature and stirred for 20 minutes. The mixture applied directly to reversed phase HPLC (5-80% acetonitrile in DI water containing 0.1% TFA, 25-minute gradient). The pure fractions were pooled and lyophilized to afford the product as a white fluffy solid. Yield 145 mg, 65%. Ion(s) found by LCMS: [M/2+H]⁺=747.2.

### Step j

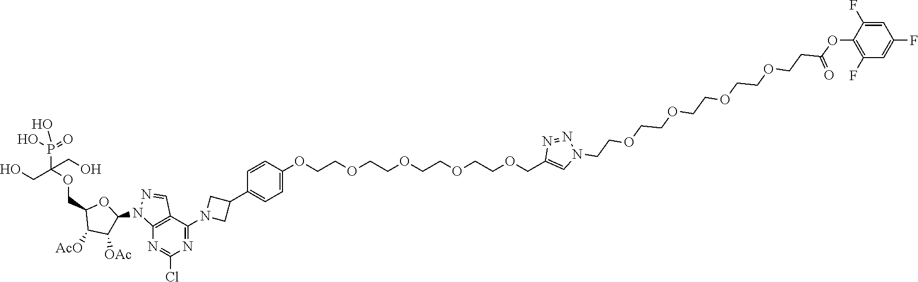 [SMILES: CC(=O)O[C@@H]1[C@H](OC(C)=O)[C@@H](COC(CO)(CO)P(=O)(O)O)O[C@H]1n1ncc2c(N3CC(c4ccc(OCCOCCOCCOCCOCc5cn(CCOCCOCCOCCOCCC(=O)Oc6c(F)cc(F)cc6F)nn5)cc4)C3)nc(Cl)nc21]

The tris benzyl protected intermediate from step i. of this example (145 mg, 0.097 mmol) was dissolved in methanol (25 mL) and the mixture was stirred in the presence of 5% Pd/C (30 mg) under 1 atmosphere of hydrogen gas for 45 minutes. The mixture was filtered through celite, concentrated and purified by reversed phase HPLC (5-80% acetonitrile in DI water containing 0.1% TFA, 25-minute gradient). The pure fractions were pooled and lyophilized to afford the product as a white fluffy solid. Yield 105 mg, 92%. Ion(s) found by LCMS: [M/2+H]⁺=612.2.

### Synthesis of Conjugate 176

Int-309 (30 mg, 0.024 mmol) was added to SEQ ID NO: 80 (130 mg, 15 mL in PBS at pH 8) as described in the synthesis of Conjugate 133b. Maldi TOF analysis of the purified final product gave an average mass of 60,155 Da. (DAR=4.3). Yield 90 mg, 70%.

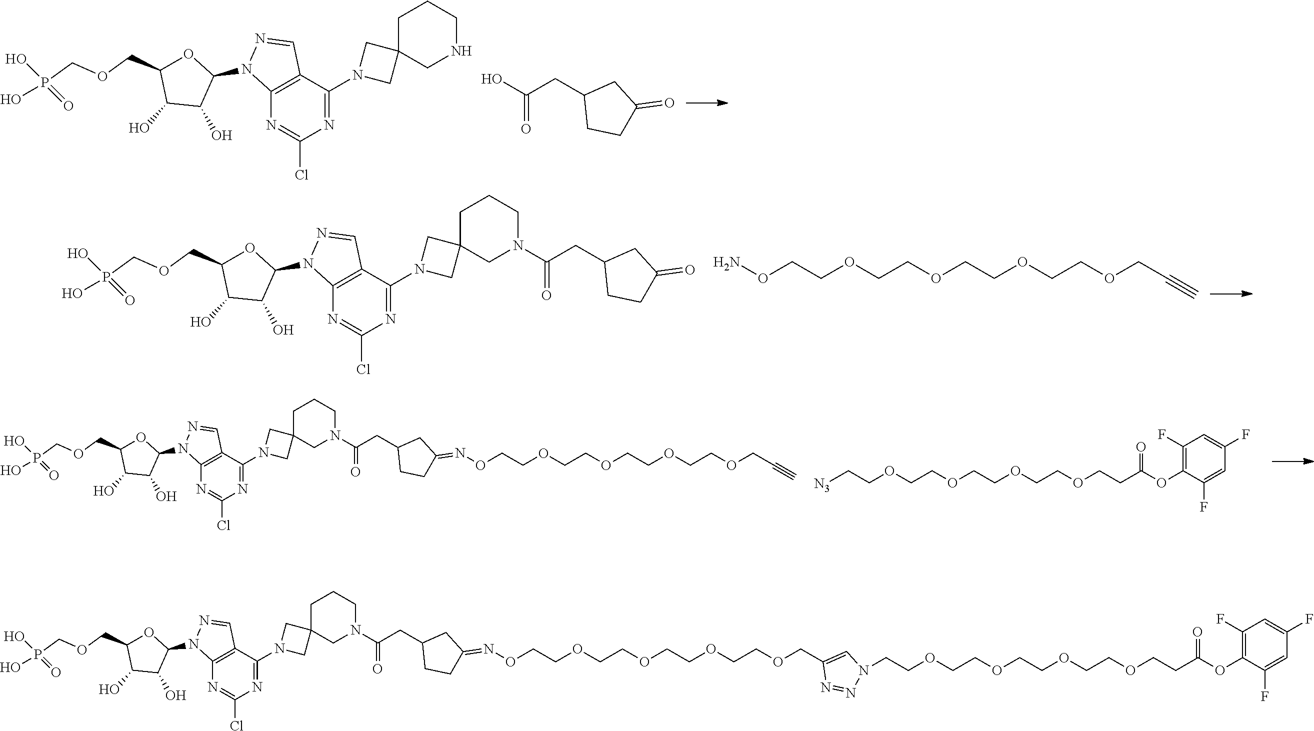

### Synthesis of Int-310

### Step a

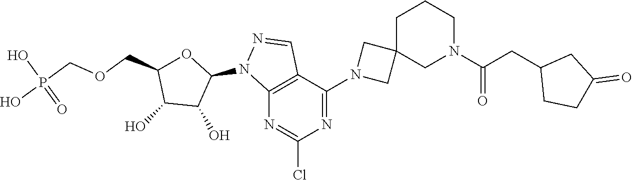 [SMILES: O=C1CCC(CC(=O)N2CCCC3(C2)CN(c2nc(Cl)nc4c2cnn4[C@@H]2O[C@H](COCP(=O)(O)O)[C@@H](O)[C@H]2O)C3)C1]

To a solution of Int-287 (80 mg, 0.15 mmol) and cyclopentane acetic acid (24 mg, 0.17 mmol) in DMF was added DIEA (0.08 mL, 0.47 mmol) and HATU (180 mg, 0.47 mmol). The reaction solution was stirred at room temperature for 2 hours. The reaction was concentrated and the residue was purified by semi-preparative HPLC (5% to 100% ACN/water, 0.1% TFA modifier) to give the product as an off-white foam. Yield 64 mg, 65%. Ion(s) found by LCMS: [M+H]⁺=629.1.

### Step b

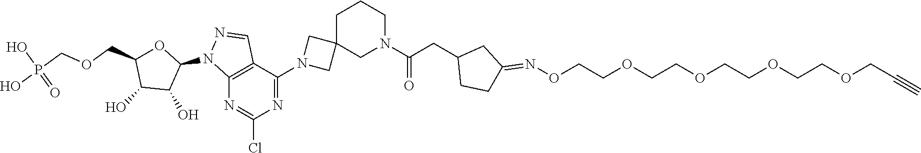 [SMILES: C#CCOCCOCCOCCOCCO/N=C1\CCC(CC(=O)N2CCCC3(C2)CN(c2nc(Cl)nc4c2cnn4[C@@H]2O[C@H](COCP(=O)(O)O)[C@@H](O)[C@H]2O)C3)C1]

To a solution of the product from the previous step (64 mg, 0.1 mmol) and aminooxy-PEG4-propargyl ether (30 mg, 0.12 mmol) in DMF (1 ml) was added DIPEA (0.053 ml, 0.3 mmol). The solution was stirred at room temperature for 20 min and purified by semi-preparative HPLC (5% to 95% acetonitrile and water, using 0.1% TFA as modifier). Yield 64 mg, 73%. Ion(s) found by LCMS: [M+H]⁺=858.3

### Step c

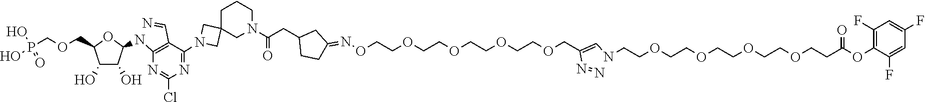 [SMILES: O=C(CCOCCOCCOCCOCCn1cc(COCCOCCOCCOCCO/N=C2\CCC(CC(=O)N3CCCC4(C3)CN(c3nc(Cl)nc5c3cnn5[C@@H]3O[C@H](COCP(=O)(O)O)[C@@H](O)[C@H]3O)C4)C2)nn1)Oc1c(F)cc(F)cc1F]

To a solution of the product from the previous step (23 mg, 0.026 mmol) and propargyl-PEG4-trifluorophenyl ester (14 mg, 0.034 mmol) in DMF (0.3 mL) was cooled to 0° C., and treated with a pre-mixed solution of a solution of copper(II) sulfate (1 mg, 0.006 mmol), sodium ascorbate (10 mg, 0.053 mmol), and THPTA (5 mg, 0.013 mmol) dissolved in water (0.3 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a white solid. Yield 22 mg, 64%. Ion(s) found by LCMS [(M+2H)/2]⁺=640.2.

### Synthesis of Conjugate 177

Int-310 (15 mg, 0.011 mmol) in DMF was added to SEQ ID NO: 80 (68 mg in 5.2 mL PBS 7.4, 0.0012 mmol) in PBS at pH 7.4 as described in the general conjugation procedure. Maldi TOF analysis of the purified final product gave an average mass of 60,666 Da. (DAR=4.8). Yield 46.2 mg, 67.9%.

### Synthesis of Int-311

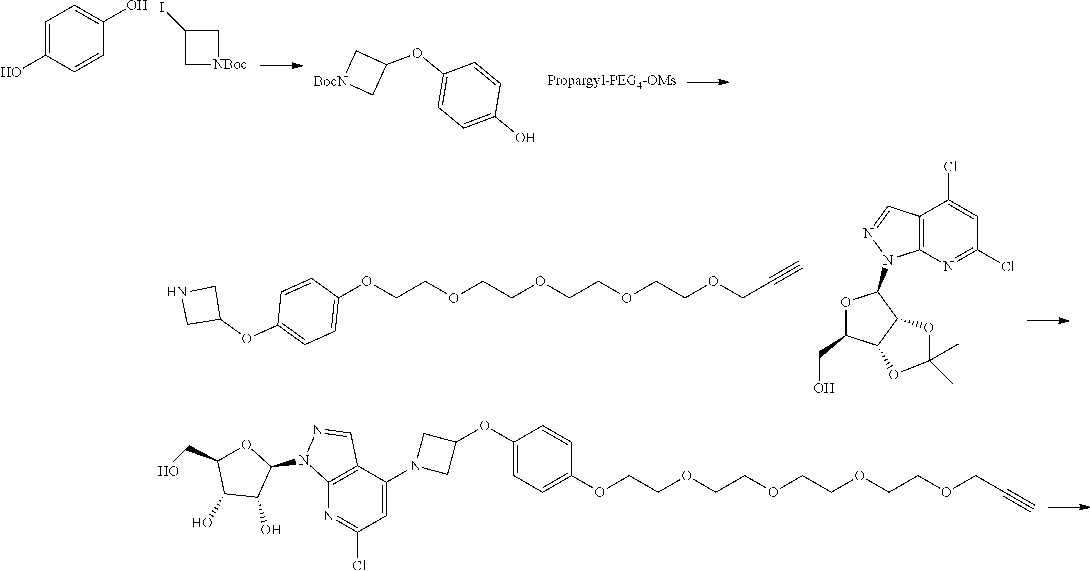

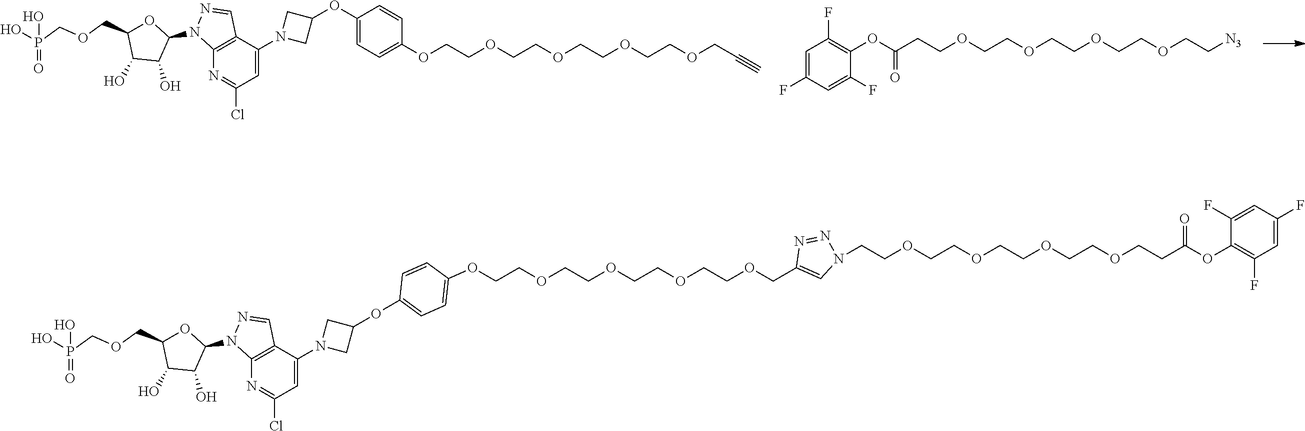

### Step a

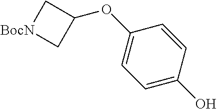 [SMILES: CC(C)(C)OC(=O)N1CC(Oc2ccc(O)cc2)C1]

Benzene-1,4-diol (3.5 g, 31.7 mmol) was added to a suspension of Cs₂CO₃ (10.36 g 31.7 mmol) in DMF (50 mL) and the mixture was heated at 60° C. for 10 minutes. The temperature was then increased to 90° C. and a solution of N-Boc-3-iodo azetidine (3 g, 10.6 mmol) in DMF (2 mL) was added dropwise. The reaction mixture was stirred for 2 hours at 90° C. The reaction was cooled to ambient temperature and diluted with water and extracted with ethyl acetate (3×20 mL). Combined organic extracts were dried over sodium sulfate, concentrated in vacuo to provide crude product, which was purified by RPLC (10% to 100% ACN/water, 0.1% TFA modifier) to yield the compound as a brown solid. Yield 2.31 g, 82%. Ion(s) found by LCMS: [M-tBu+H]+=210.2.

### Step b

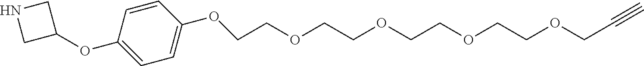 [SMILES: C#CCOCCOCCOCCOCCOc1ccc(OC2CNC2)cc1]

To a solution of the product from previous step (2.31 g, 8.7 mmol) in ACN (25 mL) was added K₂CO₃ (2.40 g, 17.414 mmol) and propargylPEG4mesylate (3.24 g, 10.43 mmol). The resulting mixture was heated at reflux overnight. Reaction mixture was cooled to ambient temperature and excess solvent was removed under reduced pressure. Ethyl acetate (50 mL) was added to the crude material and washed with water and brine. The organic layer was dried over sodium sulfate and solvent was removed under vacuum to obtain crude product which was purified by silica gel column chromatography using hexanes: ethyl acetate. Ion(s) found by LCMS: [M−Boc+H]⁺=380.2. The product was a brown viscous liquid. Yield 2.4 g, 58%. To a solution of the N-Boc derivative (2.41 g, 5 mmol) in dioxane (10 mL) was added 4M HCl solution in dioxane (20 mL). The resulting mixture was stirred at room temperature for 2 hours. Solvent was removed under reduced pressure and dried under vacuum to yield the compound as its HCl salt. The product was a brown solid. Yield 2.1 g, Quantitative. Ion(s) found by LCMS: [M-Boc+H]⁺=380.2.

### Step c

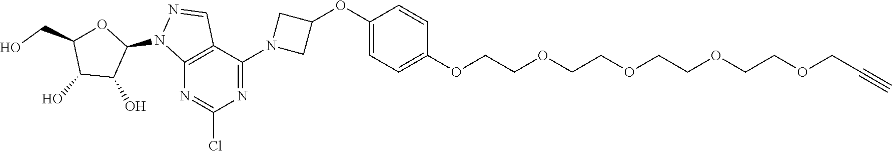 [SMILES: C#CCOCCOCCOCCOCCOc1ccc(OC2CN(c3nc(Cl)nc4c3cnn4[C@@H]3O[C@H](CO)[C@@H](O)[C@H]3O)C2)cc1]

A mixture of intermediate B (447 mg, 1 mmol), the azetidine from the previous step (498 mg, 1.2 mmol) and triethylamine (0.15 mL, 1.12 mmol) in ethanol (6 mL, 1.12 mmol) were heated at 50° C. for 1 hour. The mixture was cooled to ambient temperature and concentrated on the rotary evaporator. The crude residue was dissolved in ethyl acetate (50 mL) and washed with water and brine. The organic layer was dried over sodium sulfate, filtered and concentrated to afford the triacetate intermediate as a colorless viscous oil. Ion(s) found by LCMS: [M+H]⁺=790.2. The crude triacetate was dissolved up in methanol (5 mL), potassium carbonate (483 mg, 3.5 mmol) was added and the mixture was stirred at ambient temperature for 2 hours. The mixture was concentrated under reduced pressure. The crude residue was dissolved in ethyl acetate (50 mL) and washed with water and brine. The organic layer was dried over sodium sulfate, filtered, and concentrated to afford the crude product which was purified by RPLC (10% to 100% ACN/water, 0.1% TFA modifier) to yield the title compound as an off-white foam. Yield 580 mg, 87%. Ion(s) found by LCMS: [M+H]⁺=664.2.

### Step d

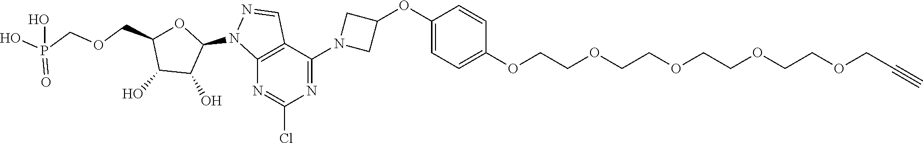 [SMILES: C#CCOCCOCCOCCOCCOc1ccc(OC2CN(c3nc(Cl)nc4c3cnn4[C@@H]3O[C@H](COCP(=O)(O)O)[C@@H](O)[C@H]3O)C2)cc1]

To a solution of the product from previous step (332 mg, 0.5 mmol) and 2,2-dimethoxypropane (156 mg, 1.5 mmol) in acetone (10 mL) at room temperature was added p-toluene sulfonic acid (86 mg, 0.5 mmol). The reaction was stirred for two hours then concentrated under reduced pressure. The crude residue was re-dissolved in EtOAc (20 mL) and washed with saturated NaHCO₃. The organic layer was dried over sodium sulfate, filtered and concentrated in vacuo to provide acetonide derivative as an off-white solid, which was used for the next step without further purification. Ion (s) found by LCMS: [M+H]⁺=704.2. A solution of acetonide derivative and [bis(tert-butoxy)carbonyl]methyl (trifluoromethyl)sulfonate (534 mg, 1.5 mmol) in dry DMF (3 mL) was cooled using ice-water bath. 60% NaH in mineral oil (120 mg, 3 mmol) was added to this and gradually warmed to room temperature and stirred for 1 h. The reaction mixture was quenched with saturated ammonium chloride then extracted with EtOAc. The organic phase was washed with brine, dried over sodium sulfate, and concentrated to give crude product which was used in the next step without purification. Ion(s) found by LCMS: [M+H]⁺=855.2. Crude product was dissolved in 50% aqueous TFA (10 mL) and stirred at room temperature for 12 hours, concentrated, and purified by semi-preparative HPLC (10-100% ACN: H₂O, 0.1% TFA modifier). The product was a white solid. Yield 190 mg, 50%. Ion(s) found by LCMS: [M+H]⁺=758.2.

### Step e

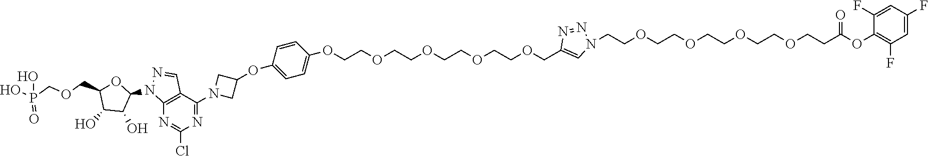 [SMILES: O=C(CCOCCOCCOCCOCCn1cc(COCCOCCOCCOCCOc2ccc(OC3CN(c4nc(Cl)nc5c4cnn5[C@@H]4O[C@H](COCP(=O)(O)O)[C@@H](O)[C@H]4O)C3)cc2)nn1)Oc1c(F)cc(F)cc1F]

To a solution of product from the previous step (150 mg, 0.198 mmol) and intermediate A (83 mg, 0.198 mmol) dissolved in DMF:H₂O (1:3, 1.5 mL) were cooled to 0° C. To this a pre-mixed solution of a solution of copper(II) sulfate (3 mg, 0.0198 mmol), sodium ascorbate (117 mg, 0.59 mmol), and BTTA (17 mg, 0.039 mmol) dissolved in water (0.5 mL) was added and stirred for 5 minutes at the same temperature and gradually warmed to room temperature and stirred for an additional 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 120 mg, 51%. Ion(s) found by LCMS [(M+2H)/2]⁺=590.2.

### Synthesis of Conjugate 178

Int-311 (41 mg, 0.027 mmol) in DMF (0.5 mL) was added to SEQ ID NO: 80 (150 mg, 11.9 mL in PBS at pH 8.4) as described in the general conjugation procedure. Maldi TOF analysis of the purified final product gave an average mass of 63,986 Da. (DAR=8.1). Yield 85 mg, 57%.

### Synthesis of Int-312

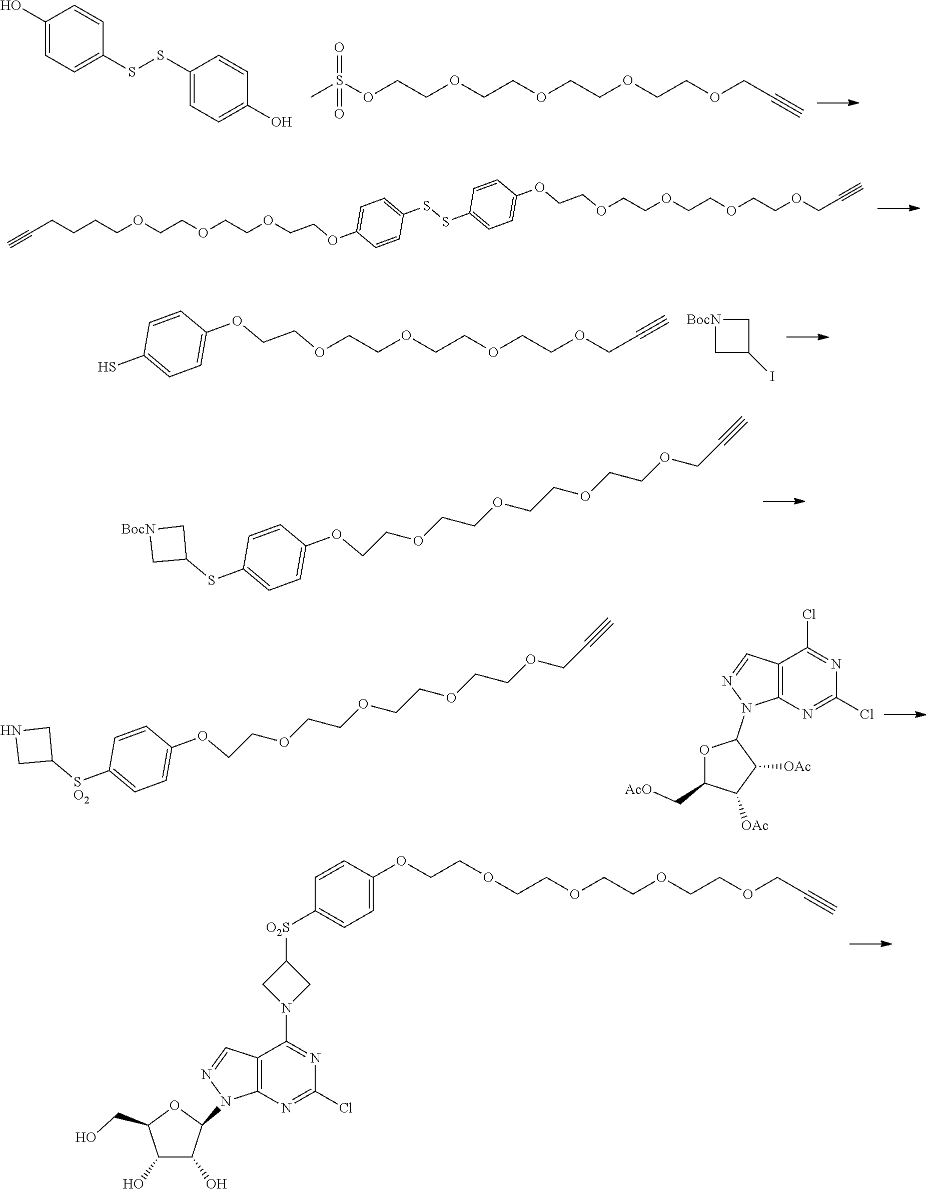

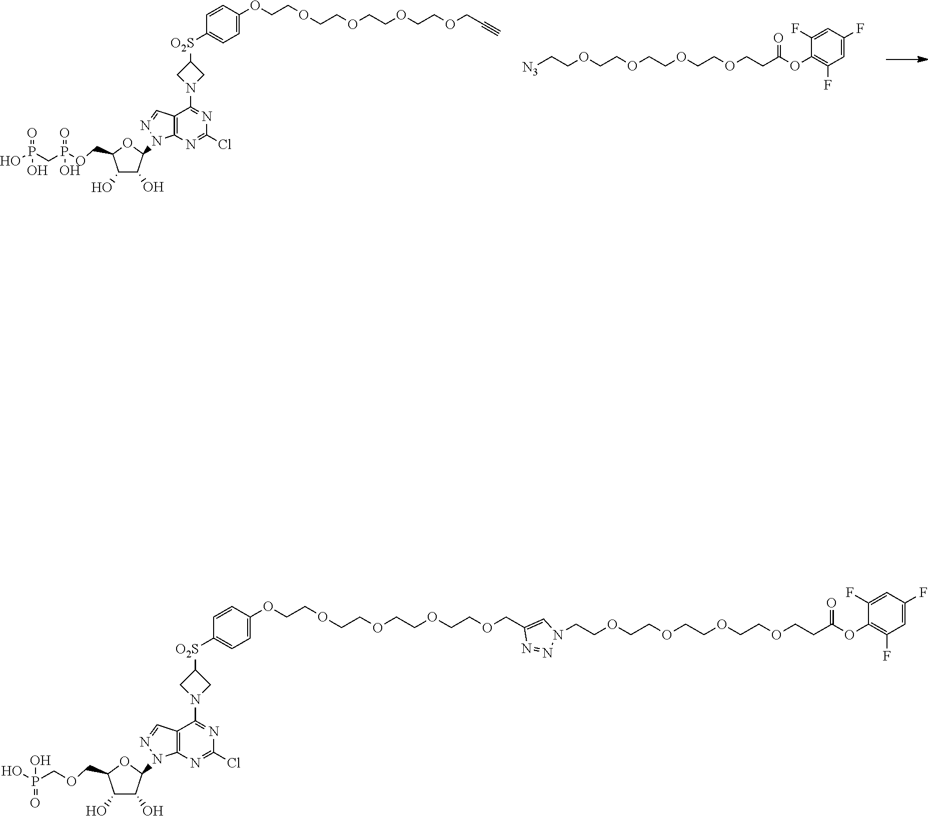 [SMILES: C#CCOCCOCCOCCOCCOc1ccc(S(=O)(=O)C2CN(c3nc(Cl)nc4c3cnn4[C@@H]3O[C@H](COP(=O)(O)CP(=O)(O)O)[C@@H](O)[C@H]3O)C2)cc1 ; [N-]=[N+]=NCCOCCOCCOCCOCCC(=O)Oc1c(F)cc(F)cc1F ; O=C(CCOCCOCCOCCOCCn1cc(COCCOCCOCCOCCOc2ccc(S(=O)(=O)C3CN(c4nc(Cl)nc5c4cnn5[C@@H]4O[C@H](COCP(=O)(O)O)[C@@H](O)[C@H]4O)C3)cc2)nn1)Oc1c(F)cc(F)cc1F]

### Step a

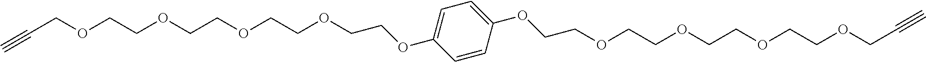 [SMILES: C#CCOCCOCCOCCOCCOc1ccc(OCCOCCOCCOCCOCC#C)cc1]

Commercially available 4-[(4-hydroxyphenyl)disulfanyl]phenol (1 g, 4 mmol), propargylPEG₄mesylate (2.5 g, 7.99 mmol) and potassium carbonate (2.2 g, 15.97 mmol) were dissolved in acetonitrile (50 mL) and heated at 80° C. for 16 h. Reaction mixture was cooled to room temperature and solvent was removed by rotary evaporation. Crude residue was partitioned between water and ethyl acetate. Organic layer collected and aqueous layer extracted with EtOAc (2×20 mL). Combined organic extracts were washed with water, brine and dried over sodium sulfate. Removal of the solvent followed by purification by RPLC (10% to 100% ACN/water, 0.1% TFA modifier) yielded product as a yellow viscous oil. Yield 2.4 g, 87%. Ions found by LCMS: [M+H]⁺=679.2.

### Step b

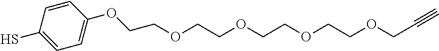 [SMILES: C#CCOCCOCCOCCOCCOc1ccc(S)cc1]

To a solution of the disulfide product from previous step (2.35 g, 3.46 mmol) in anhydrous THE (50 mL) in a three-neck round-bottom flask with a nitrogen inlet, reflux condenser, and nitrogen inlet, was added Bu₃P (1.4 g, 46.92 mmol) in portions. The reaction was stirred for 10 minutes and allowed to cool below 50° C. H₂O (10 mL) was added and the mixture was stirred for 60 minutes. The aqueous layer was discarded, and the organic layer was extracted with 10% NaOH (3×25 mL). The alkaline aqueous extract was washed with toluene (3×20 mL), acidified with dilute HCl and extracted with CH₂Cl₂ (2×15 mL). The organic layer was dried over sodium sulfate and the solvent was evaporated under reduced pressure to give pure arylthiol as a light-yellow viscous oil, which was used for the next step without any further purification. Yield 1 g, 85%. Ion(s) found by LCMS: [M+H]⁺=341.2.

### Step c

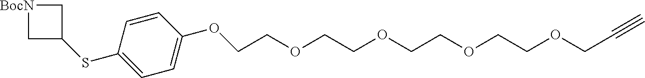 [SMILES: C#CCOCCOCCOCCOCCOc1ccc(SC2CN(C(=O)OC(C)(C)C)C2)cc1]

To a stirred solution of thiophenol derivative from previous step (1 g, 2.94 mmol) in DMF (25 mL) was added Cs₂CO₃ (1.91 g, 5.87 mmol) followed by 1-Boc-3-iodoazetidine (0.831 g, 2.94 mmol). The reaction was stirred overnight then diluted with water and EtOAc. Organic layer was collected, and the aqueous layer was extracted with EtOAc (2×20 mL). Combined organic extracts were washed with water, saturated NaHCO₃ solution and brine. The organic layer was dried over sodium sulfate and concentrated to give an oil which was purified by RPLC (10% to 100% ACN/water, 0.1% TFA modifier). The product was a light yellow viscous oil. Yield 1.1 g, 75%. Ion(s) found by LCMS: [M-tBu+H]+=440.2.

### Step d

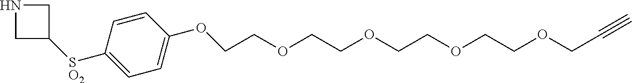 [SMILES: C#CCOCCOCCOCCOCCOc1ccc(S(=O)(=O)C2CNC2)cc1]

To a solution of the sulfide product from previous step (1.1 g, 2.22 mmol) in DCM (20 mL) at 0° C. was added mCPBA (1.15 g, 6.66 mmol). The resulting mixture was warmed to room temperature and stirred for 2 hours. Reaction mixture was washed with saturated NaHCO₃ solution, water, brine and dried over sodium sulfate. Filtration followed by removal of the organic solvent by rotary evaporation yielded the crude material which was purified by RPLC (10% to 100% ACN/water, 0.1% TFA modifier). The product was a light yellow viscous liquid. Yield 620 mg, 53%. Ions found by LCMS: [M−Boc+H]⁺=428.2. Boc amine (620 mg, 1.18 mmol) was treated with 4M HCl in dioxane (5 mL) at room temperature for 4 hours. Removal of the solvent followed by vacuum drying yielded the compound title compound as HCl salt. The product was a light yellow viscous liquid. Yield 560 mg, quantitative yield. Ions found by LCMS: [M+H]⁺=428.2.

### Step e

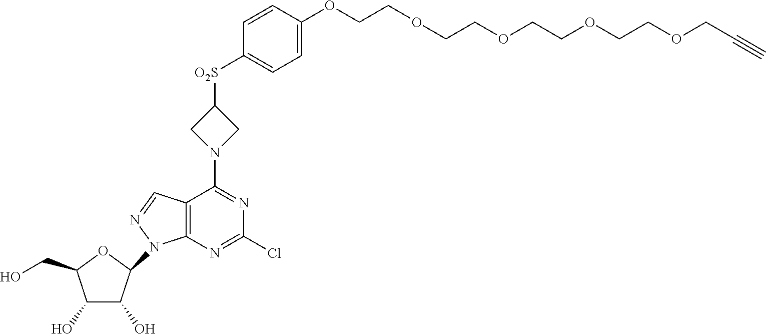 [SMILES: C#CCOCCOCCOCCOCCOc1ccc(S(=O)(=O)C2CN(c3nc(Cl)nc4c3cnn4[C@@H]3O[C@H](CO)[C@@H](O)[C@H]3O)C2)cc1]

A mixture of intermediate B (313 mg, 0.7 mmol), the amine from the previous step (359 mg, 0.84 mmol), and diisopropylethylamine (0.195 mL, 1.4 mmol) in methanol (5 mL) were heated at 50° C. for 1 hour. After complete consumption of the starting materials, the reaction mixture was cooled to room temperature and volatiles were removed by rotatory evaporation. The crude residue was redissolved in ethyl acetate (50 mL) and washed with water and brine. The organic layer was dried over sodium sulfate, filtered and solvent was removed under reduced pressure to yield the triacetate intermediate, which was used for the next step without further purification. Ions found by LCMS: [M+H]⁺=838.2. The triacetate intermediate was re-dissolved in methanol (4 mL) and potassium carbonate (338 mg, 2.45 mmol) was added, and the reaction mixture was stirred at ambient temperature for 2 hours. The reaction mixture was filtered and concentrated to get crude product which was purified by reverse phase HPLC (5% to 100% ACN/water, 0.1% TFA modifier) to yield the compound. Off-white foam. Yield 380 mg, 76%. Ion(s) found by LCMS: [M+H]⁺=712.2.

### Step f

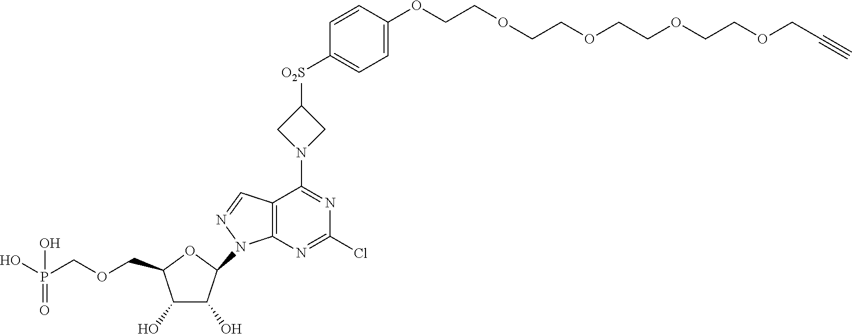 [SMILES: C#CCOCCOCCOCCOCCOc1ccc(S(=O)(=O)C2CN(c3nc(Cl)nc4c3cnn4[C@@H]3O[C@H](COCP(=O)(O)O)[C@@H](O)[C@H]3O)C2)cc1]

To a solution of the triol from previous step (300 mg, 0.421 mmol) and 2,2-dimethoxypropane (132 mg, 1.26 mmol) in acetone (10 mL) at room temperature was added p-toluene sulfonic acid (72 mg, 0.421 mmol). The reaction was stirred for two hours then concentrated under reduced pressure. The crude material was dissolved in EtOAc (20 mL) and washed with saturated NaHCO₃. The organic layer was separated and dried over sodium sulfate, filtered, and concentrated in vacuo to provide crude acetonide derivative, which was purified by flash chromatography (DCM: 10% MeOH in DCM) to yield corresponding acetonide derivative as an off-white foam. Yield 237 mg, 75%. Ion(s) found by LCMS: [M+H]⁺=752.2. A solution of the acetonide derivative (237 mg, 0.315 mmol) and [bis(tert-butoxy)carbonyl]methyl (trifluoromethyl)sulfonate (336 mg, 0.945 mmol) in dry DMF (3 mL) was cooled using ice-water bath. 60% NaH in mineral oil (80 mg, 1.89 mmol) was added to this and gradually warmed to room temperature and stirred for 1 hour. The reaction mixture was quenched with saturated ammonium chloride then extracted with EtOAc. The organic phase was washed with brine, dried over sodium sulfate, and concentrated to give crude product which was used in the next step without purification. Ion(s) found by LCMS: [M+H]⁺=846.2. Crude product was dissolved in 50% aqueous TFA (10 mL) and stirred at room temperature for 12 hours, concentrated, and purified by semi-preparative HPLC (10-100% ACN: H₂O, 0.1% TFA modifier). The product was a white solid. Yield 170 mg, 67%. Ion(s) found by LCMS: [M+H]⁺=806.2.

### Step g

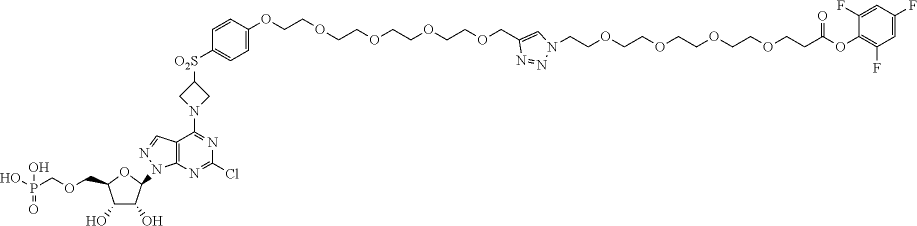 [SMILES: O=C(CCOCCOCCOCCOCCn1cc(COCCOCCOCCOCCOc2ccc(S(=O)(=O)C3CN(c4nc(Cl)nc5c4cnn5[C@@H]4O[C@H](COCP(=O)(O)O)[C@@H](O)[C@H]4O)C3)cc2)nn1)Oc1c(F)cc(F)cc1F]

To a solution of product from The previous step (50 mg, 0.0.06 mmol) and intermediate A (26 mg, 0.062 mmol). dissolved in DMF:H₂O (1:3, 1.5 mL) were cooled to 0° C. To this a pre-mixed solution of a solution of copper(II) sulfate (0.9 mg, 0.006 mmol), sodium ascorbate (37 mg, 0.186 mmol), and BTTA (5 mg, 0.0124 mmol) dissolved in water (0.5 mL) was added and stirred for 5 minutes at the same temperature and gradually warmed to room temperature and stirred for an additional 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 39 mg, 51%. Ion(s) found by LCMS [(M+2H)/2]⁺=614.2.

Synthesis of Conjugate 179 Int-312 (12.4 mg, 0.010 mmol in DMF (0.5 mL) was added to SEQ ID NO: 80 (70 mg, 5.42 mL in PBS at pH 8.4) as described in the general conjugation procedure. Maldi TOF analysis of the purified final product gave an average mass of 60,422 Da. (DAR=4.8). Yield 41 mg, 58%.

### Synthesis of Int-314

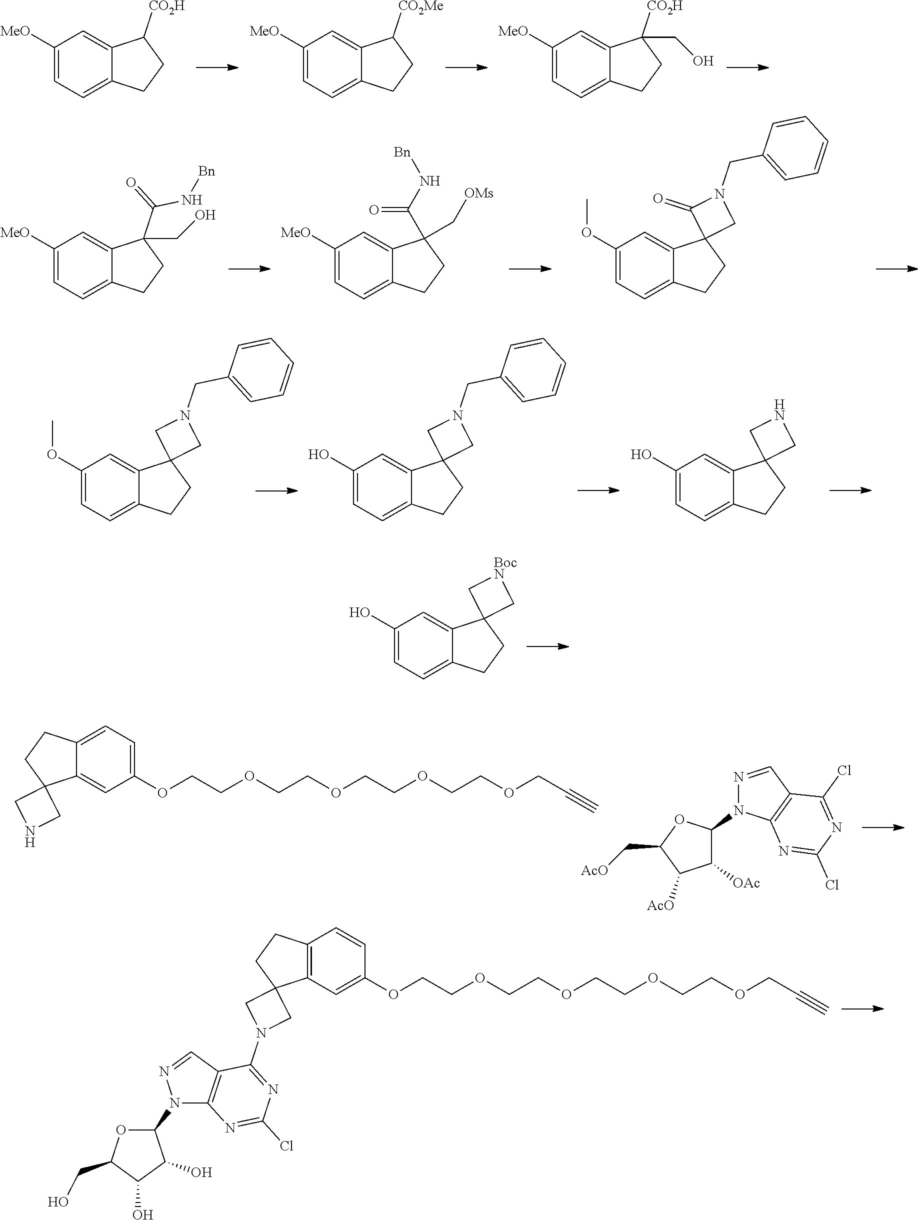

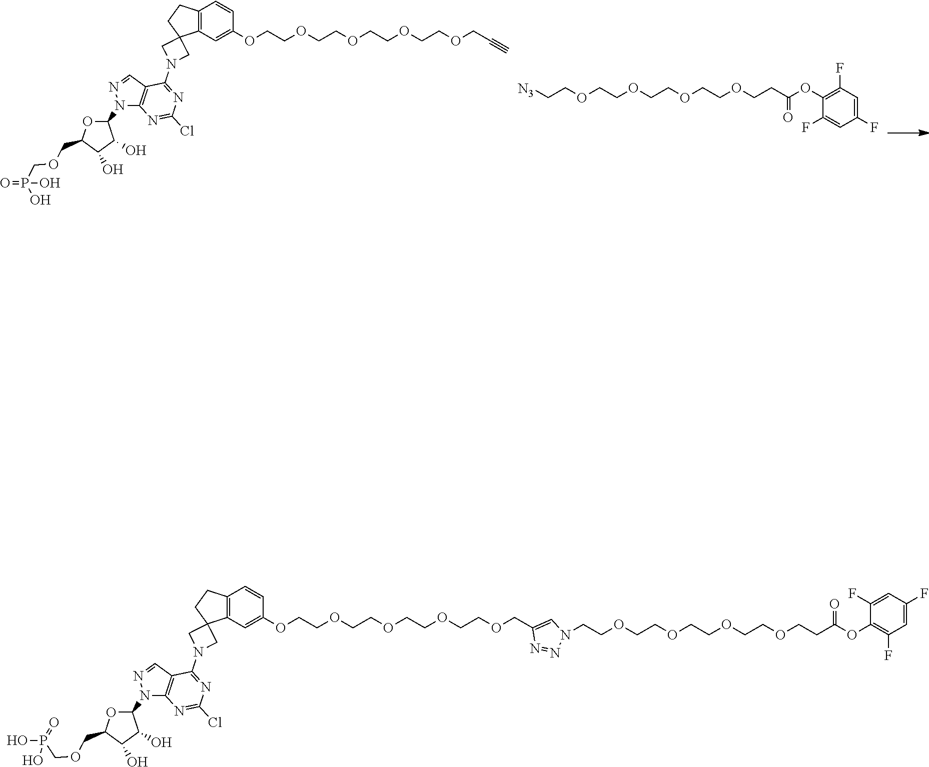 [SMILES: C#CCOCCOCCOCCOCCOc1ccc2c(c1)C1(CC2)CN(c2nc(Cl)nc3c2cnn3[C@@H]2O[C@H](COCP(=O)(O)O)[C@@H](O)[C@H]2O)C1 ; [N-]=[N+]=NCCOCCOCCOCCOCCC(=O)Oc1c(F)cc(F)cc1F ; O=C(CCOCCOCCOCCOCCn1cc(COCCOCCOCCOCCOc2ccc3c(c2)C2(CC3)CN(c3nc(Cl)nc4c3cnn4[C@@H]3O[C@H](COCP(=O)(O)O)[C@@H](O)[C@H]3O)C2)nn1)Oc1c(F)cc(F)cc1F]

### Step a

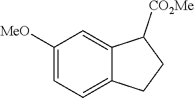 [SMILES: COC(=O)C1CCc2ccc(OC)cc21]

A dried round-bottom flask was charged with 6-methoxy-2,3-dihydro-IH-indene-1-carboxylic acid (1.0 g, 5.2 mmol) and MeOH (10 mL). H₂SO₄ (1 mL) at room temperature was added to the methanolic solution of the compound. The resulting reaction mixture was stirred at 50° C. for 3 hours. The reaction mixture was cooled to room temperature and concentrated. Crude material was re-dissolved in EtOAc (25 mL) and washed with saturated NaHCO₃ solution and brine. Organic phase was dried over sodium sulfate and concentrated by rotavapor and purified by column chromatography on silica-gel with 0-25% EtOAc in hexane to give the desired product as a clear viscous liquid. Yield 1.07 g, 99%. Ion(s) found by LCMS [M+H]⁺=207.2.

### Step b

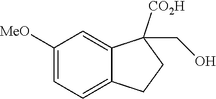 [SMILES: COc1ccc2c(c1)C(CO)(C(=O)O)CC2]

A mixture of the carboxylate from previous step (1.07 g, 5.188 mmol), formaldehyde (820 mg, 10.376 mmol, 38% in water) and potassium carbonate (2.15 g, 15.564 mmol) in DMSO (20 mL) was stirred at room temperature for 16 hours. Then the mixture was poured into ice water (50 mL) and extracted with EtOAc (3×25 mL). The combined organic phases were dried over anhydrous sodium sulfate, filtered, and concentrated in vacuo. The resulting residue was purified by column chromatography on silica gel (EtOAc:hexanes=1:2) to afford the title compound as a clear viscous oil. Yield 1.1 g, 94%. Ion(s) found by LCMS [M−H]⁺=221.2.

### Step c

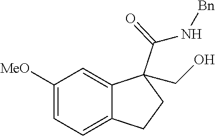 [SMILES: COc1ccc2c(c1)C(CO)(C(=O)NCc1ccccc1)CC2]

To a solution of the carboxylic acid from previous step (1.08, 4.86 mmol) in DMF (10 mL) was added benzylamine (625 mg, 5.83 mmol) DIPEA (1.27 mL, 7.28 mmol) and HATU (2.77 g, 7.29 mmol). The reaction mixture was stirred at room temperature for 16 hours and partitioned between DCM and water. The organic layers were washed with brine (25 mL), dried over anhydrous sodium sulfate, filtered, and concentrated in vacuo. The residue was purified by column chromatography on silica gel (10% MeOH in DCM) to afford compound the compound as a viscous oil. Yield 960 mg, 63%. Ion(s) found by LCMS [M+H]⁺=312.2.

### Step d

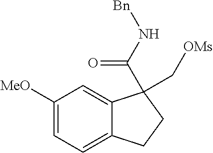 [SMILES: COc1ccc2c(c1)C(COS(C)(=O)=O)(C(=O)NCc1ccccc1)CC2]

To a solution of product from the previous step (960 mg, 3.08 mmol) in DCM (20 mL) was added triethylamine (0.65 mL, 4.62 mmol) and methanesulfonyl chloride (0.36 mL, 4.62 mmol) at 0-5° C. After stirring at room temperature for 12 hours, the reaction mixture was poured into ice water and extracted with DCM (3×25 mL). The combined organic phases were washed with saturated aqueous NaHCO₃ solution, brine, dried over anhydrous sodium sulfate, and concentrated in vacuo. The residue was purified by column chromatography on silica gel (hexanes:EtOAc=6:1) to give intermediate mesylate. Light yellow viscous oil. 1.1 g, 90%. Ion(s) found by LCMS [M+H]⁺=390.2.

### Step e

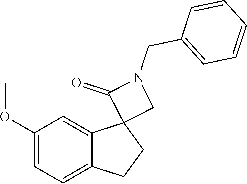 [SMILES: COc1ccc2c(c1)C1(CC2)CN(Cc2ccccc2)C1=O]

To a solution of product from previous step (1.08 g, 2.77 mmol) in acetonitrile (25 mL) was added potassium carbonate (764 mg, 5.54 mmol). After 24 hours, stirring at 80° C., the mixture was poured into ice water (50 mL) and extracted with EtOAc (3×25 mL). The combined organic phases were dried over anhydrous sodium sulfate, filtered, and concentrated in vacuo. The residue was purified by column chromatography on silica gel (EtOAc:hexanes=1:2) to afford the compound as a yellow viscous oil. Yield 780 mg, 96%. Ion(s) found by LCMS [M+H]⁺=294.2.

### Step f

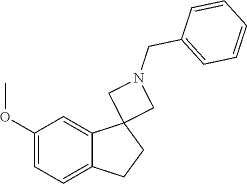 [SMILES: COc1ccc2c(c1)C1(CC2)CN(Cc2ccccc2)C1]

To a solution of aluminum chloride (532 mg, 3.99 mmol) in THE (10 mL) was added lithium aluminum hydride (3.98 mL, 2M solution in THF, 7.98 mmol) at 0° C. The mixture was stirred at 0° C. for 30 minutes. To the mixture was added a solution of product from previous step (780 mg, 2.66 mmol) in THE (5 mL) dropwise at 0° C. The reaction mixture was stirred at ambient temperature for 1 hour, before adding water (25 mL), aqueous sodium hydroxide solution (15%, 25 mL) and water (75 mL) dropwise at 0° C. in sequence. After 10 minutes stirring at 0° C., mixture EtOAc (50 mL) was added to the mixture and filtered through celite. The organic layer of the filtrate was separated, and the aqueous layer was extracted with EtOAc (2×50 mL). The organic layers were combined, washed with water (50 mL), brine (50 mL) and dried over anhydrous sodium sulfate, filtered, and concentrated in vacuo. The residue was purified by column chromatography on silica gel (EtOAc:hexanes=1:1) to afford the compound as a yellow viscous liquid. 680 mg, 92%. Ion(s) found by LCMS [M+H]⁺=280.2.

### Step g

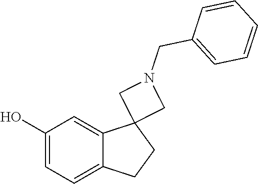 [SMILES: Oc1ccc2c(c1)C1(CC2)CN(Cc2ccccc2)C1]

To a solution of the compound from the previous step (680 mg, 2.43 mmol) in DCM (25 mL) at 0° C. was added boron tribromide (0.46 mL, 4.86 mmol) in dropwise. The resulting mixture was gradually warmed to room temperature and stirred until LCMS showed completion of the reaction (30 minutes), reaction mixture was cooled to 0° C. and carefully quenched by the addition of water. Organic layer was collected, and aqueous layer was extracted with EtOAc (3×20 mL). Combined extracts were washed with water, brine and dried over anhydrous sodium sulfate, filtered. Removal of the solvent yielded crude product which was purified by RPLC (10% to 100% ACN/water, 0.1% TFA modifier). The product was a white solid. 580 mg, 90%. Ion(s) found by LCMS [M+H]⁺=266.2.

### Step h

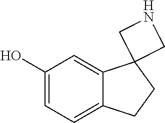 [SMILES: Oc1ccc2c(c1)C1(CC2)CNC1]

To a solution of 1-benzyl-2′,3′-dihydrospiro[azetidine-3,r-indene from the previous step (580 mg, 2.185 mmol) in methanol (20 mL) was added dihydroxypalladium (306 mg, 0.437 mmol), and ammonium formate (0.165 g, 2.62 mmol). The reaction mixture was stirred under a balloon of hydrogen gas at 60° C. for 4 hours. The reaction mixture was filtered, washed with methanol (3×30 mL). The filtrate was concentrated in vacuo to get the crude product as an off-white solid, which was used for the next step without any further purification. Yield 380 mg, quantitative. Ion(s) found by LCMS [M+H]⁺=176.2.

### Step i

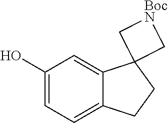 [SMILES: CC(C)(C)OC(=O)N1CC2(CCc3ccc(O)cc32)C1]

DMAP (26 mg, 0.22 mmol) and Boc₂O (720 mg, 3.3 mmol) were added to a solution of the phenol derivative from previous step (380 mg, 2.2 mmol) dissolved in DMF (5 mL). The resulting mixture was stirred at room temperature for 1 hour (Reaction monitored by LCMS) and the solvent was removed by rotary evaporation to get the crude material which was purified by silica gel column chromatography (DCM-10% MeOH in DCM). The product was an off-white foam. Yield 496 mg, 81%. Ion(s) found by LCMS [M-tBu+H]+=220.2.

### Step j

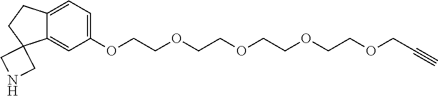 [SMILES: C#CCOCCOCCOCCOCCOc1ccc2c(c1)C1(CC2)CNC1]

The compound from the step i (490 mg, 1.78 mmol), propargyl-PEG4 mesylate (663 mg, 2.13 mmol) and cesium carbonate (1.15 g, 3.56 mmol) were dissolved in acetonitrile (20 mL) and heated at reflux for 4 hours. The reaction mixture was cooled to room temperature and the solvent was removed by rotary evaporator. Then the mixture was poured into water (50 mL) and extracted with EtOAc (3×25 mL). The combined organic phases were dried on anhydrous sodium sulfate, filtered, and concentrated in vacuo to give a residue, which was purified by RPLC (10% to 100% ACN/water, 0.1% TFA modifier). The product was a colorless viscous oil. 678 mg, 78%. Ions (s) found by LCMS: [M−^{t}Bu+H]⁺=434.2. N-Boc deprotected compound (678 mg, 1.39 mmol) dissolved in DCM (5 mL) and TFA (5 mL) was added to it. The resulting mixture was stirred at room temperature for 6 hours. Removal of the solvent followed by vacuum drying yielded the compound title compound as TFA salt. Yield 690 mg, Quantitative. Ions found by LCMS: [M+H]⁺=390.2.

### Step k

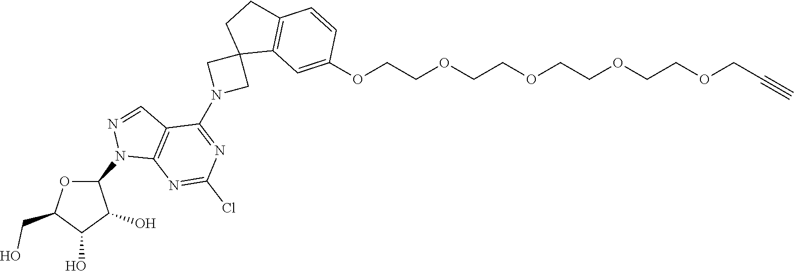 [SMILES: C#CCOCCOCCOCCOCCOc1ccc2c(c1)C1(CC2)CN(c2nc(Cl)nc3c2cnn3[C@@H]2O[C@H](CO)[C@@H](O)[C@H]2O)C1]

A mixture of intermediate B (223 mg, 0.5 mmol), amine from the previous step (302 mg, 0.6 mmol) and diisopropylethylamine (0.21 mL, 1.5 mmol) in methanol (6 mL, 1.12 mmol) were heated at 50° C. for 1 hour. The mixture was cooled to ambient temperature and concentrated on the rotary evaporator. The crude residue was dissolved in ethyl acetate (50 mL) and washed with water and brine. The organic layer was dried over sodium sulfate, filtered, and concentrated to afford the triacetate intermediate as a colorless viscous oil. Ion(s) found by LCMS: [M+H]⁺=809.2. The crude triacetate was dissolved up in methanol (5 mL), potassium carbonate (242 mg, 1.75 mmol) was added, and the mixture was stirred at ambient temperature for 2 hours. The mixture was concentrated under reduced pressure The crude residue was dissolved in ethyl acetate (50 mL) and washed with water and brine. The organic layer was dried over sodium sulfate, filtered, and concentrated to afford the crude product which was purified by RPLC (10% to 100% ACN/water, 0.1% TFA modifier). The product was a white solid. Yield 280 mg, 83%. Ion(s) found by LCMS: [M+H]⁺=674.2.

### Step l

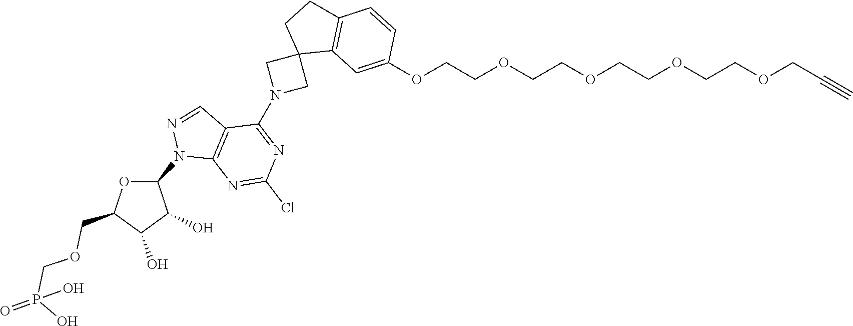 [SMILES: C#CCOCCOCCOCCOCCOc1ccc2c(c1)C1(CC2)CN(c2nc(Cl)nc3c2cnn3[C@@H]2O[C@H](COCP(=O)(O)O)[C@@H](O)[C@H]2O)C1]

To a solution of the product from previous step (280 mg, 0.42 mmol) and 2,2-dimethoxypropane (130 mg, 1.24 mmol) in acetone (10 mL) at room temperature was added p-toluene sulfonic acid (72 mg, 0.42 mmol). The reaction was stirred for two hours then concentrated under reduced pressure. The crude residue was dissolved in EtOAc (20 mL) and washed with saturated NaHCO₃. The organic layer was collected and dried over sodium sulfate, filtered, and concentrated in vacuo to provide acetonide derivative as an off-white foam, which was used for the next step without further purification. Yield 300 mg, Quantitative. Ion(s) found by LCMS: [M+H]⁺=714.1. A solution of acetonide derivative (300 mg, 0.42 mmol) and [bis(tert-butoxy)carbonyl]methyl (trifluoromethyl)sulfonate (444 mg, 1.25 mmol) in dry DMF (3 mL) was cooled using ice-water bath. 60% NaH in mineral oil (99 mg, 2.49 mmol) was added to this and gradually warmed to room temperature and stirred for 1 hour. The reaction mixture was quenched with saturated ammonium chloride and extracted with EtOAc. The organic phase was washed with brine, dried over sodium sulfate, and concentrated to give crude product which was used in the next step without purification. Ion(s) found by LCMS: [M−2^{t}Bu+H]+=808.2. Crude product was dissolved in 50% aqueous TFA (10 mL) and stirred at room temperature for 12 hours, concentrated, and purified by semi-preparative HPLC ((10% to 100% ACN/water, 0.1% TFA modifier)). The product was a white solid. Yield 110 mg, 34%. Ion(s) found by LCMS: [M+H]⁺=714.1.

### Step m

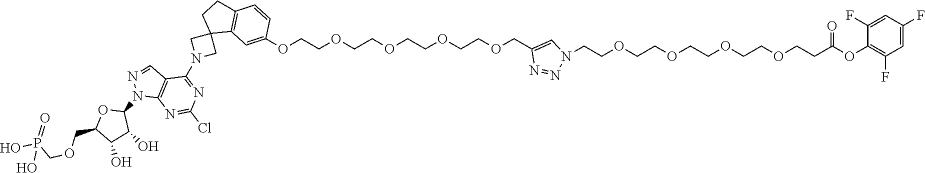 [SMILES: O=C(CCOCCOCCOCCOCCn1cc(COCCOCCOCCOCCOc2ccc3c(c2)C2(CC3)CN(c3nc(Cl)nc4c3cnn4[C@@H]3O[C@H](COCP(=O)(O)O)[C@@H](O)[C@H]3O)C2)nn1)Oc1c(F)cc(F)cc1F]

A solution of the product from the previous step (70 mg, 0.091 mmol) and intermediate A (38 mg, 0.091 mmol) were dissolved in DMF:H₂O (1:3, 1.5 mL) and subsequently cooled to 0° C. To this a pre-mixed solution of a solution of copper(II) sulfate (1.5 mg, 0.0091 mmol), sodium ascorbate (54 mg, 0.273 mmol), and BTTA (7 mg, 0.018 mmol) dissolved in water (0.5 mL) was added and stirred for 5 minutes at the same temperature and gradually warmed to room temperature and stirred for an additional 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 64 mg, 59%. Ion(s) found by LCMS [(M+2H)/2]⁺=595.2.

### Synthesis of Conjugate 181

Int-314 (22 mg, 0.022 mmol in DMF (0.5 mL) was added to SEQ ID NO: 80 (120 mg, 9.52 mL in PBS at pH 8.4) as described in the general conjugation procedure. Maldi TOF analysis of the purified final product gave an average mass of 61,191 Da. (DAR=5.7). Yield 60 mg, 57%.

### Synthesis of Int-315

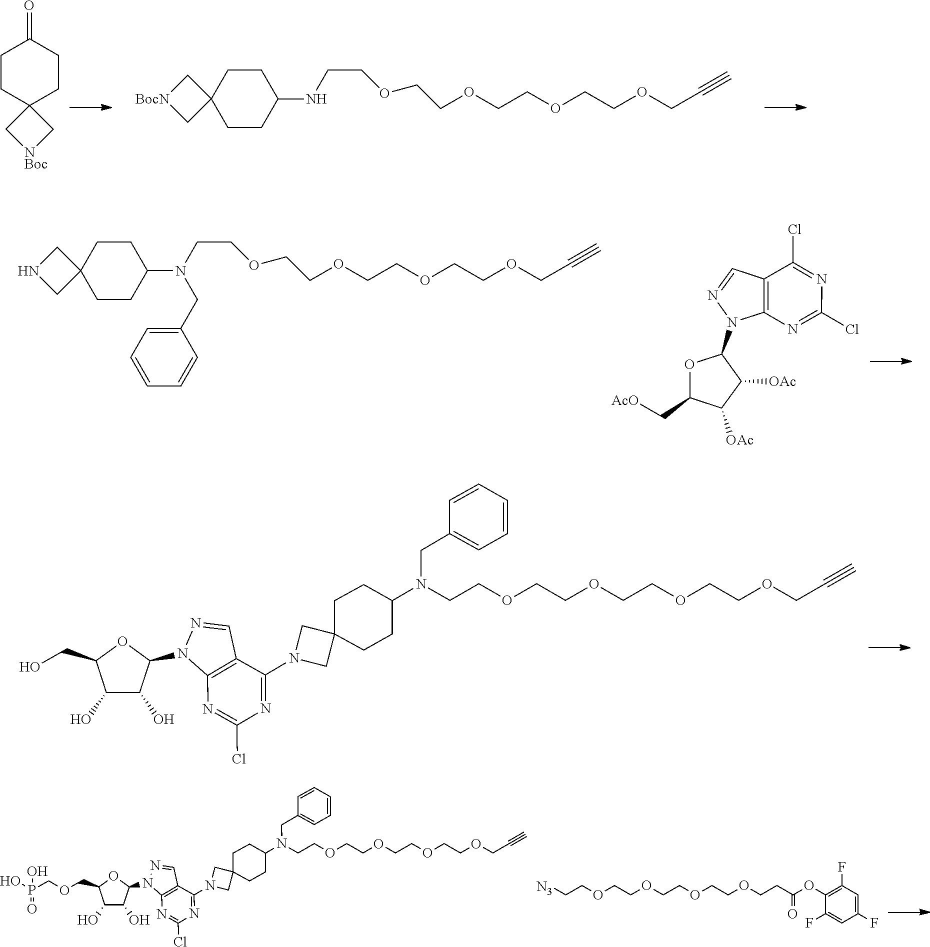

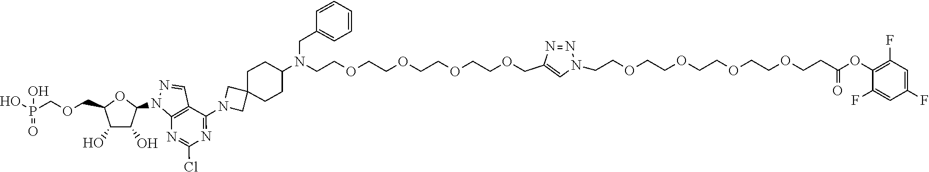 [SMILES: O=C(CCOCCOCCOCCOCCn1cc(COCCOCCOCCOCCN(Cc2ccccc2)C2CCC3(CC2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

### Step a

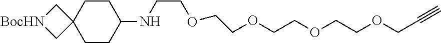

t-Butyl 7-oxo-2-azaspiro[3.5]nonane-2-carboxylate (478 mg, 2 mmol) and propargyl-peg₄-amine (462 mg, 2 mmol) in THE (10 mL) was stirred at ambient temperature for 16 hours. Sodium borohydride (114 mg, 3 mmol) was added to the mixture and the reaction was stirred at room temperature for 1 hour. Reaction mixture was quenched by the addition of MeOH and concentrated, residue thus obtained was purified by RPLC (10% to 100% ACN/water, 0.1% TFA modifier) to yield the title compound as a light-yellow viscous oil. Yield 772 mg, 85%. Ion(s) found by LCMS [M+H]⁺=455.4.

### Step b

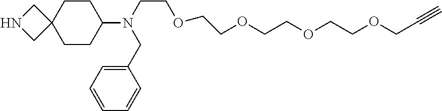 [SMILES: C#CCOCCOCCOCCOCCN(Cc1ccccc1)C1CCC2(CC1)CNC2]

To a solution of the product previous step (772 mg, 1.7 mmol) in DMF (5 mL) was added benzyl bromide (0.30 mL, 2.56 mmol) and diisopropylethylamine (0.45 mL, 2.56 mmol). The reaction mixture was stirred at room temperature for 16 hours and concentrated in vacuo. The residue was purified by RPLC (ACN: H₂O, 0.1% TFA modifier) to yield the title compound as a viscous oil. Yield 786 mg, 84.99%. Ion(s) found by LCMS [M+H]⁺=545.2. N-Boc deprotected compound (786 mg, 1.39 mmol) dissolved in DCM (5 mL) and TFA (8 mL) was added to it. The resulting mixture was stirred at room temperature for 6 hours. Removal of the solvent followed by vacuum drying yielded the compound title compound as TFA salt. Yield 774 mg, Quantitative. Ions found by LCMS: [M+H]⁺=445.2.

### Step c

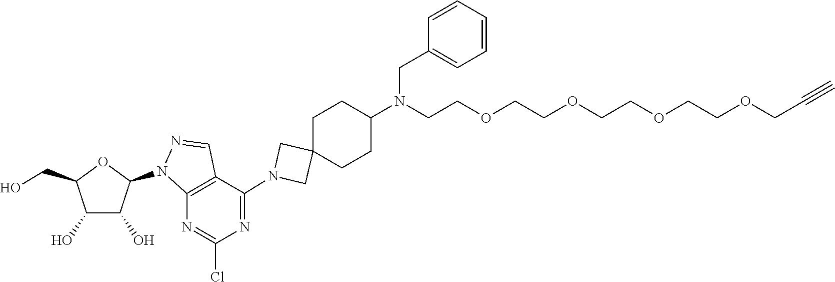 [SMILES: C#CCOCCOCCOCCOCCN(Cc1ccccc1)C1CCC2(CC1)CN(c1nc(Cl)nc3c1cnn3[C@@H]1O[C@H](CO)[C@@H](O)[C@H]1O)C2]

A mixture of intermediate B (447 mg, 1 mmol), amine from the previous step (533 mg, 1.2 mmol) and diisopropylethylamine (0.42 mL, 3 mmol) in methanol (15 mL) were heated at 50° C. for 1 hour. The mixture was cooled to ambient temperature and concentrated on the rotary evaporator. The crude residue was dissolved in ethyl acetate (50 mL) and washed with water and brine. The organic layer was dried over sodium sulfate, filtered, and concentrated to afford the triacetate intermediate as a colorless viscous oil. Ion(s) found by LCMS: [M+H]⁺=855.4. The crude triacetate was dissolved up in methanol (10 mL), potassium carbonate (484 mg, 3.5 mmol) was added, and the mixture was stirred at ambient temperature for 2 hours. The mixture was concentrated under reduced pressure. The crude residue was dissolved in ethyl acetate (50 mL) and washed with water and brine. The organic layer was dried over sodium sulfate, filtered, and concentrated to afford the crude product which was purified by RPLC (10% to 100% ACN/water, 0.1% TFA modifier). The product was a white solid. Yield 623 mg, 85%. Ion(s) found by LCMS: [M+H]⁺=729.2.

### Step d

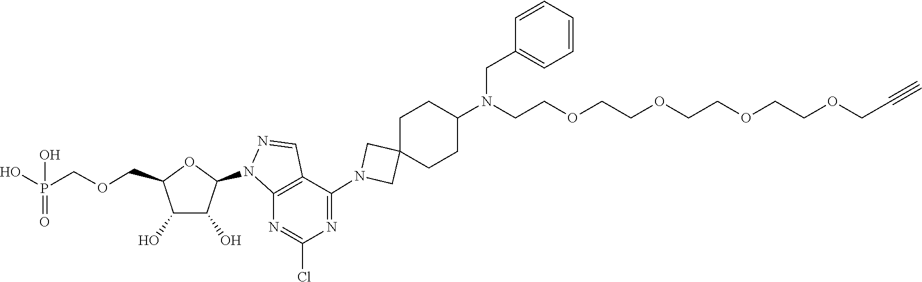 [SMILES: C#CCOCCOCCOCCOCCN(Cc1ccccc1)C1CCC2(CC1)CN(c1nc(Cl)nc3c1cnn3[C@@H]1O[C@H](COCP(=O)(O)O)[C@@H](O)[C@H]1O)C2]

To a solution of the product from previous step (364 mg, 0.5 mmol) and 2,2-dimethoxypropane (156 mg, 1.5 mmol) in Acetone (10 mL) at room temperature was added p-toluene sulfonic acid (86 mg, 0.5 mmol). The reaction was stirred for two hours then concentrated under reduced pressure. The crude residue was re-dissolved in EtOAc (20 mL) and washed with saturated NaHCO₃. The organic layer was collected and dried over sodium sulfate, filtered and concentrated in vacuo to provide acetonide derivative as an off-white foam, which was used for the next step without further purification. Yield 300 mg, quantitative. Ion(s) found by LCMS: [M+H]⁺=769.4. A solution of acetonide derivative and [bis(tert-butoxy)phosphoryl]methyl (trifluoromethyl)sulfonate (534 mg, 1.5 mmol) in dry DMF (3 mL) was cooled using ice-water bath. 60% NaH in mineral oil (120 mg, 3 mmol) was added to this and gradually warmed to room temperature and stirred for 1 hour. The reaction mixture was quenched with saturated ammonium chloride and extracted with EtOAc. The organic phase was washed with brine, dried over sodium sulfate, and concentrated to give crude product which was used in the next step without purification. Ion(s) found by LCMS: [M+H]⁺=976.2. The crude product was dissolved in 50% aqueous TFA (10 mL) and stirred at room temperature for 12 hours, concentrated, and purified by semi-preparative HPLC (10% to 100% ACN/water, 0.1% TFA modifier). The product was a white solid. Yield 283 mg, 69%. Ion(s) found by LCMS: [M+H]⁺=823.2.

### Step e

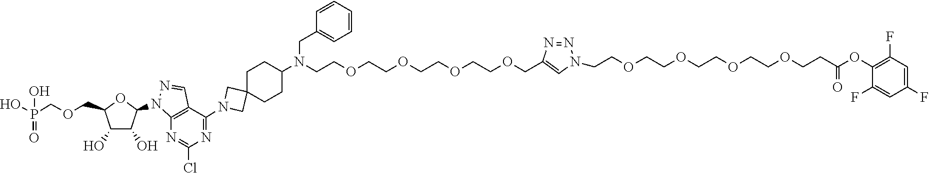 [SMILES: O=C(CCOCCOCCOCCOCCn1cc(COCCOCCOCCOCCN(Cc2ccccc2)C2CCC3(CC2)CN(c2nc(Cl)nc4c2cnn4[C@@H]2O[C@H](COCP(=O)(O)O)[C@@H](O)[C@H]2O)C3)nn1)Oc1c(F)cc(F)cc1F]

To a solution of product from the previous step (70 mg, 0.085 mmol) and intermediate A (36 mg, 0.085 mmol). dissolved in DMF:H₂O (1:3, 1.5 mL) were cooled to 0° C. To this a pre-mixed solution of a solution of copper(II) sulfate (1.3 mg, 0.008 mmol), sodium ascorbate (551 mg, 0.255 mmol), and BTTA (7.3 mg, 0.017 mmol) dissolved in water (0.5 mL) was added and stirred for 5 minutes at the same temperature and gradually warmed to room temperature and stirred for an additional 15 minutes. When the reaction was complete (by HPLC), the mixture was quenched with a few drops of glacial acetic acid and 125 mM EDTA (pH 6) and the product was purified by reversed phase HPLC (10% to 100% ACN/water, 0.1% TFA modifier). The product was a white solid. Yield 65 mg, 61%. Ion(s) found by LCMS [(M+2H)/2]⁺=622.6.

### Synthesis of Conjugate 182

Int-315 (22 mg, 0.0181 mmol in DMF (0.5 mL) was added to SEQ ID NO: 80 (100 mg, 7.93 mL in PBS at pH 8.4) as described in the general conjugation procedure. Maldi TOF analysis of the purified final product gave an average mass of 64,996 Da. (DAR=8.9). Yield 67 mg, 56%.

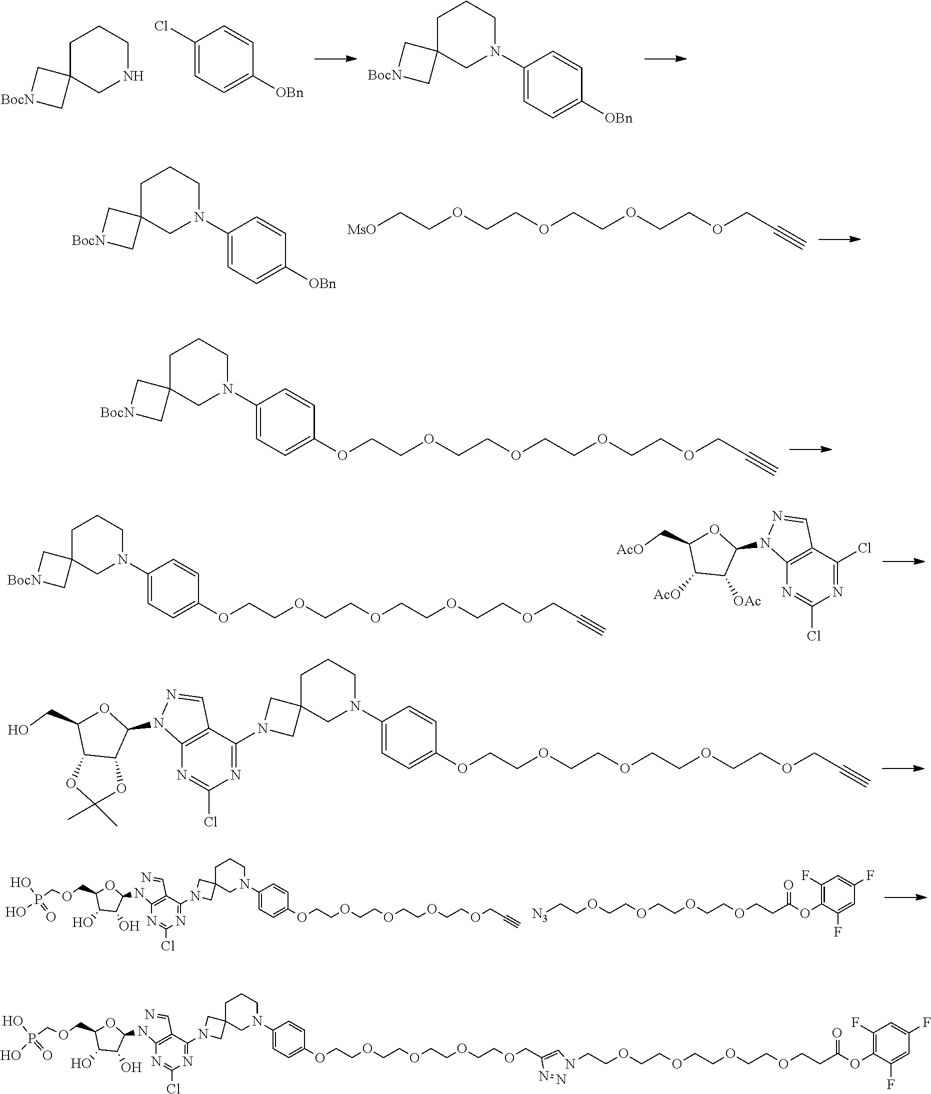

### Step a

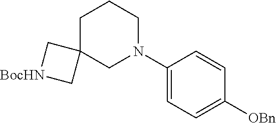

To a solution of t-butyl piperazine-1-carboxylate (0.45 g, 2 mmol) in toluene (40 ml) was added p-benzyloxy-bromobenzene (0.43 g, 2 mmol) Pd₂(dba)₃ (0.09 g, 0.10 mmol) and X-Phos (0.095 g, 0.20 mmol) at room temperature and heated to 50° C. for 15 minutes under nitrogen. Sodium t-butoxide (0.57 g, 6 mmol) was added to the reaction mixture and the reaction mixture was heated at 100° C. for 1 hour. On completion of reaction, the reaction mixture was filtered through celite bed and washed with ethyl acetate. The combined organic layers were washed with water, dried over anhydrous sodium sulfate and concentrated under reduced pressure. The resultant residue was purified by column chromatography using an ethyl acetate-hexane mixture as eluent to afford the pure compound. Yield 0.61 g, 74%. Ion(s) found by LCMS: [M+H]⁺=551.2.

### Step b

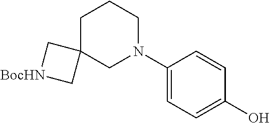

To a solution of step-a product (0.55 g, 1.4 mmol) in methanol (15 ml) was added Pd(OH)₂ (0.2 g). The reaction solution was stirred under an H₂ atmosphere overnight, filtered through a celite, washed with DCM and concentrated to give the crude which was used in the next step without purification. Yield 0.45 g, 100%. Ion(s) found by LCMS: [M+H]⁺=319.1.

### Step c

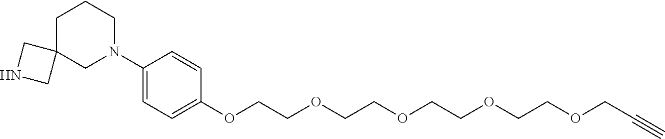 [SMILES: C#CCOCCOCCOCCOCCOc1ccc(N2CCCC3(CNC3)C2)cc1]

To a solution of the product from the previous step (0.42 g, 1.34 mmol) and propargyl-PEG4-mesylate in acetonitrile (5 mL) was added potassium carbonate (0.55 g, 4.0 mmol). The reaction solution at 70° C. was stirred at room temperature for 2 hours, filtered and concentrated. The residue was purified by semi-preparative HPLC (ACN: H2O; 0 to 100%). Yield 0.51 g, 71%. Ion(s) found by LCMS: [M+H]⁺=533.3. Intermediate N-Boc derivative was dissolved in DCM (10 mL) and TFA (4 mL). The resulting mixture was stirred for 4 h at room temperature then solvent was removed under reduced pressure and dried under vacuum to yield the desired compound as its TFA salt. The product was a yellow oil. Yield 480 mg, 100%. Ion(s) found by LCMS: [M+H]⁺=432.2.

### Step d

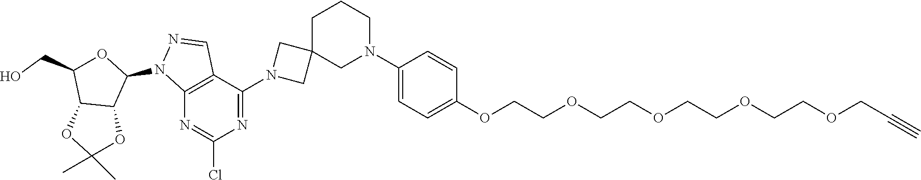 [SMILES: C#CCOCCOCCOCCOCCOc1ccc(N2CCCC3(C2)CN(c2nc(Cl)nc4c2cnn4[C@@H]2O[C@H](CO)[C@H]4OC(C)(C)O[C@H]42)C3)cc1]

A solution of triacetate intermediate B (0.44 g, 0.9 mmol), spirocyclic azetidine from the previous step (0.48 mg, 0.9 mmol), and triethylamine (0.18 mL, 1.35 mmol) in anhydrous EtOH (mL) was stirred at room temperature for 30 min. The solution was concentrated, and the residue was dissolved in 7 M NH3/MeOH (7 mL). The reaction was stirred at room temperature overnight and mixture was concentrated to dryness under reduced pressure, then dissolved in acetone (1.3 mL). 2,2-dimethoxypropane (1.3 mL, 10 mmol) and p-TsOH (0.21 g, 1.1 mmol) was added and the mixture was stirred overnight at room temperature for 2 hours. The reaction mixture was concentrated to dryness under reduced pressure and purified by flash chromatography (DCM: 10% MeOH in DCM) to yield product. The product was a white foam. Yield 0.48 mg, 70%. Ion(s) found by LCMS: [M+H]⁺=757.3.

### Step e

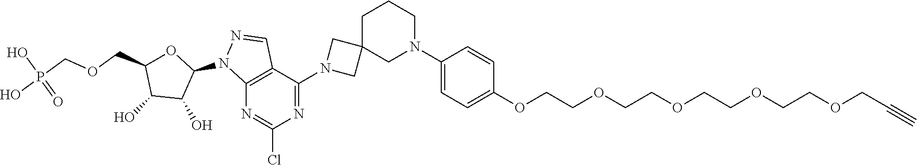 [SMILES: C#CCOCCOCCOCCOCCOc1ccc(N2CCCC3(C2)CN(c2nc(Cl)nc4c2cnn4[C@@H]2O[C@H](COCP(=O)(O)O)[C@@H](O)[C@H]2O)C3)cc1]

A solution of acetonide derivative from previous step (0.12 g, 0.16 mmol) and [bis(tert-butoxy)carbonyl]methyl (trifluoromethyl)sulfonate (0.18 g, 0.5 mmol) in dry DMF was cooled using ice-water bath. 60% NaH in mineral oil (0.04 g, 1 mmol) was added to this and stirred at room temperature for 2 h. The reaction mixture was quenched with saturated ammonium chloride and extracted with EtOAc.

The organic phase was washed with brine and dried over sodium sulfate concentrated to give crude product which was used in the next step without purification. Crude product was dissolved in 50% aqueous TFA (3 mL) and stirred at room temperature for 12 h, concentrated and the residue was purified by semi-preparative HPLC (10-100% ACN: H₂O, 0.1% TFA modifier). The product was a white solid. Yield 0.056 g, 40%. Ion(s) found by LCMS: [M+H]⁺=811.2.

### Step f

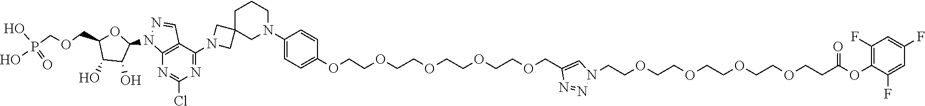 [SMILES: O=C(CCOCCOCCOCCOCCn1cc(COCCOCCOCCOCCOc2ccc(N3CCCC4(C3)CN(c3nc(Cl)nc5c3cnn5[C@@H]3O[C@H](COCP(=O)(O)O)[C@@H](O)[C@H]3O)C4)cc2)nn1)Oc1c(F)cc(F)cc1F]

To a solution of the product from the previous step (50 mg, 0.061 mmol) and propargyl-PEG4-trifluorophenyl ester (0.033 g, 0.08 mmol) dissolved in DMF (0.6 mL) was cooled to 0° C., and treated with a pre-mixed solution of a solution of copper(II) sulfate (2 mg, 0.015 mmol), sodium ascorbate (24 mg, 0.123 mmol), and THPTA (13 mg, 0.03 mmol) in water (0.6 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). Yield 56 mg, 73%. Ion(s) found by LCMS [(M+2H)/2]⁺=616.7.

### Synthesis of Conjugate 183

Int-316 (16 mg, 0.013 mmol) in DMF was added to SEQ ID NO: 80 (83 mg in 6.6 mL PBS 7.4, 0.0015 mmol) in PBS at pH 7.4 as described in the general conjugation procedure. Maldi TOF analysis of the purified final product gave an average mass of 62,524 Da. (DAR=6.7). Yield 49.6 mg, 60.4%.

### Synthesis of Int-323

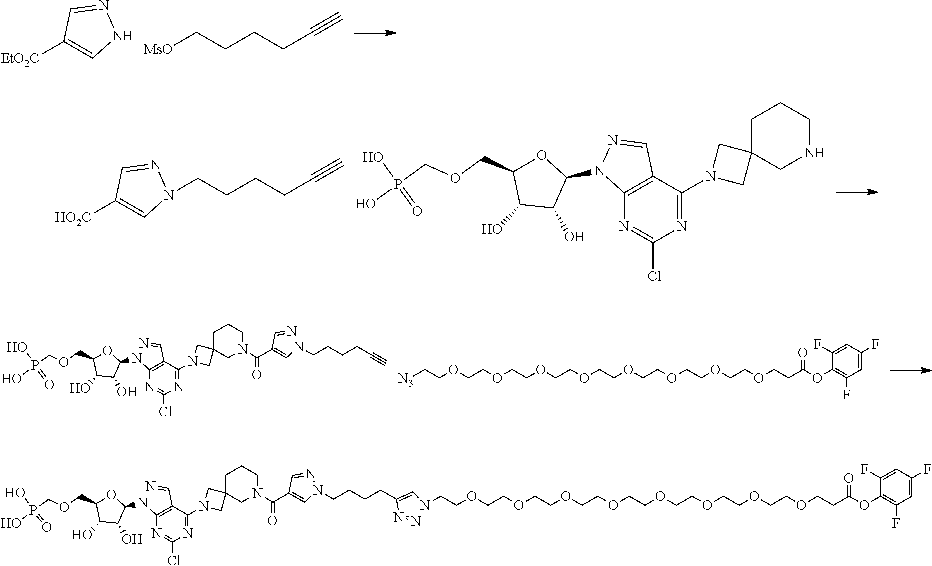

### Step a

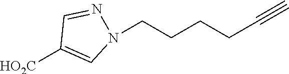

To a solution of 5-Hexyn-1-yl mesylate(4.1 g, 23 mmol, preparation described in “Synthesis of Int-305”) and ethyl 2H-pyrazole-4-carboxylate (2.2 g, 16 mmol) in anhydrous DMF (10 ml) was added K₂CO₃ (2.1 g, 16 mmol). The reaction mixture was heated at 70° C. overnight. After cooling to room temperature, the K₂CO₃/salt was filtered off and washed with acetonitrile. The filtrate was concentrated, and the residue was dissolved in MeOH (10 ml) and treated with a solution of LiOH monohydrate (0.75 g, 30 mmol) in water (10 ml). The mixture was stirred at room temperature for 2 hours, then acidified with TFA and residue was purified by RPLC (5% to 100% ACN/water, 0.1% TFA modifier) to give the desired acid as a solid. Yield 2.1 g, 69%. Ion(s) found by LCMS: [M+H]⁺=193.0.

### Step b

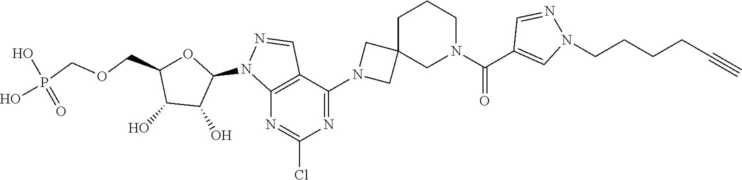 [SMILES: C#CCCCCn1cc(C(=O)N2CCCC3(C2)CN(c2nc(Cl)nc4c2cnn4[C@@H]2O[C@H](COCP(=O)(O)O)[C@@H](O)[C@H]2O)C3)cn1]

To a solution of spirocyclic piperidine (100 mg, 0.2 mmol) (described in “Synthesis of Int-287”) and pyrazole-acid (38 mg, 0.2 mmol) in DMF (2 ml) was added DIEA (0.2 mL, 1.2 mmol) and HATU (110 mg, 0.3 mmol). The reaction solution was stirred at room temperature for 2 hours. The reaction solution was diluted with water and extracted with EtOAc. The organic phase was concentrated and purified by semi-preparative HPLC (5% to 100% ACN/water, 0.1% TFA modifier) to give the product as an off-white foam. Yield 81 mg, 61%. Ion(s) found by LCMS: [M+H]⁺=679.2.

### Step c

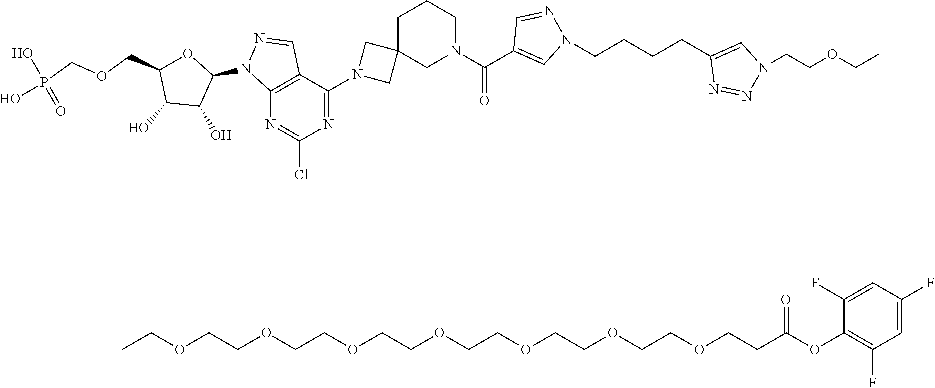

To a solution of the product from the previous step (50 mg, 0.07 mmol) and azido-PEG8-trifluorophenyl ester (prepared as described in step a of “Synthesis of Int-282”) (52 mg, 0.08 mmol) in DMF (0.6 mL) was cooled to 0° C., and treated with a pre-mixed solution of a solution of copper(II) sulfate (5.8 mg, 0.03 mmol), sodium ascorbate (43 mg, 0.22 mmol), and BTTAA (15 mg, 0.03 mmol) water (0.6 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a colorless oil.

Yield 62 mg, 65%. Ion(s) found by LCMS [(M+2H)/2]⁺=639.4.

### Synthesis of Conjugate 190

Int-323 (33 mg, 0.02 mmol) in DMF (10 mL) was added to SEQ ID NO: 80 (120 mg in 6.7 mL PBS 7.4, 0.002 mmol) in PBS at pH 7.4 as described in the general conjugation procedure. Maldi TOF analysis of the purified final product gave an average mass of 63,032 Da. (DAR=7). Yield 81 mg, 68%.

### Synthesis of Int-335

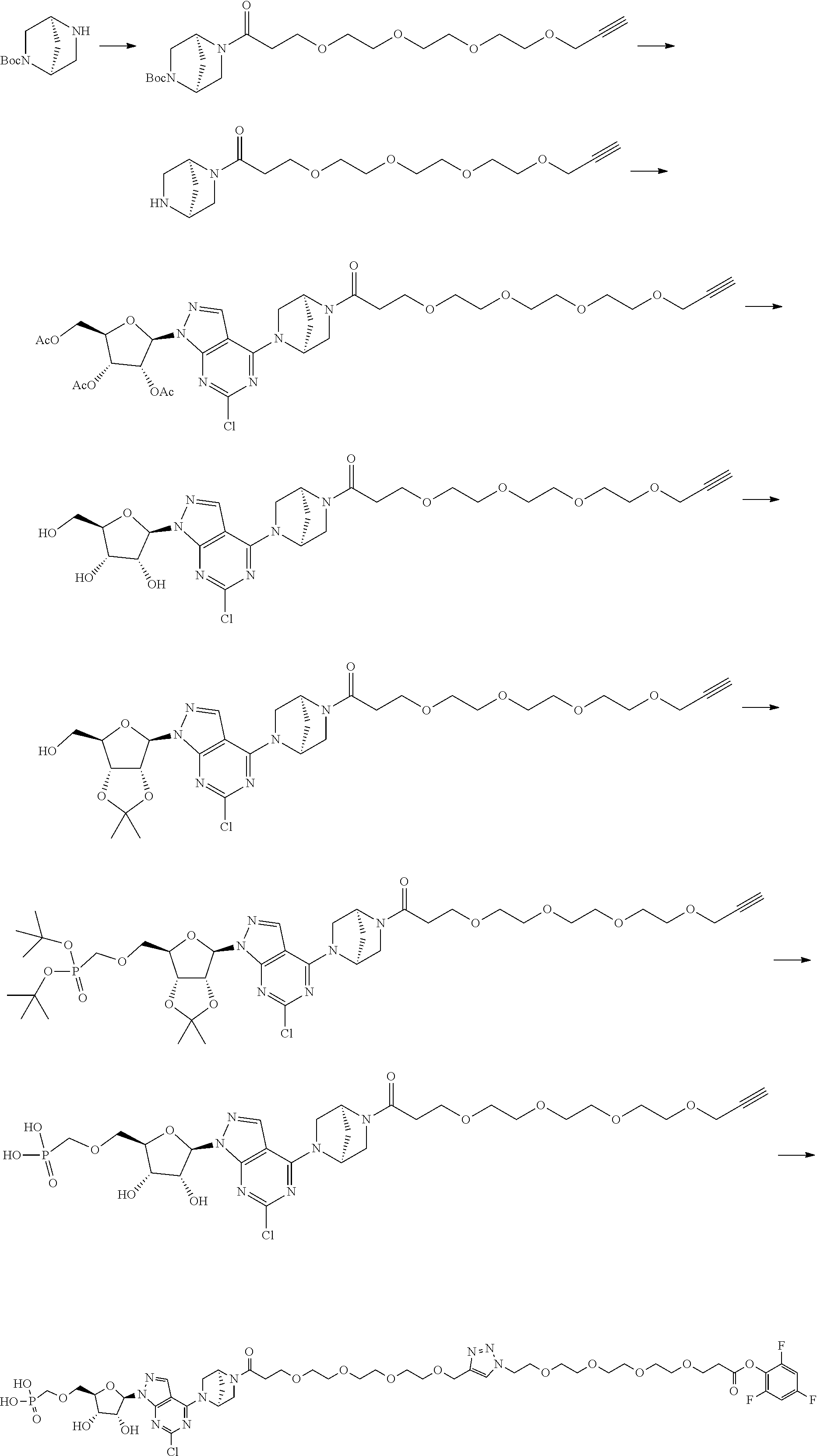

### Step a

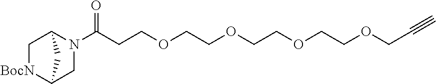 [SMILES: C#CCOCCOCCOCCOCCC(=O)N1C[C@@H]2C[C@H]1CN2C(=O)OC(C)(C)C]

To a solution of a mixture of propargyl-PEG4-acid (520.6 mg, 2.0 mmol) and HATU (912.5 mg, 2.4 mmol) in anhydrous DMF (2.5 ml) was added (1S,4S)-2-Boc-2,5-diazabicyclo[2.2.1]heptane (436.3 mg, 2.2 mmol) and DIPEA (387.7 mg, 3 mmol). The reaction mixture was stirred for 1 hour, then directly purified by RPLC (100 g, 5 to 100% acetonitrile and water). Yield 803 mg, 91.1%. Ions found by LCMS: [M+H]⁺=441.2

### Step b

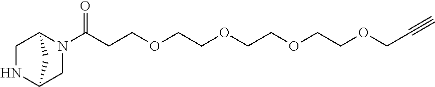 [SMILES: C#CCOCCOCCOCCOCCC(=O)N1C[C@@H]2C[C@H]1CN2]

Product from step a (803 mg, 1.823 mmol) was dissolved in acetonitrile (3 ml) and treated with 6N aqueous HCl solution (1 ml). The reaction mixture was heated at 50° C. for 45 minutes. It then concentrated by rotary evaporation and further dried under high vacuum. The crude product was carried to the subsequent step without purification. Ion found by LCMS: [M+H]⁺=341.2.

### Step c

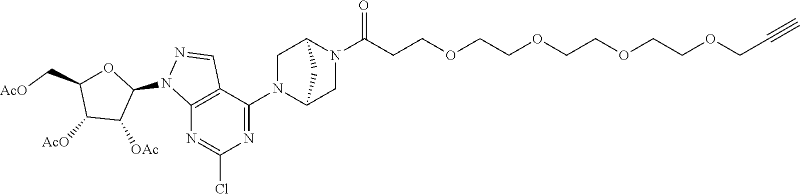 [SMILES: C#CCOCCOCCOCCOCCC(=O)N1C[C@@H]2C[C@H]1CN2c1nc(Cl)nc2c1cnn2[C@@H]1O[C@H](COC(C)=O)[C@@H](OC(C)=O)[C@H]1OC(C)=O]

To a solution of Intermediate B (178.9 mg, 0.4 mmol) in anhydrous DMF (0.4 ml) was added product from step b (188.5 mg, 0.5 mmol), DMF (0.4 ml) and DIPEA (129.2 mg, 1 mmol). The reaction mixture was stirred at room temperature for 1 hour and then carried to subsequent step without purification. Ions found by LCMS: [M+Na]⁺=773.2, [M+H]⁺=751.2.

### Step d

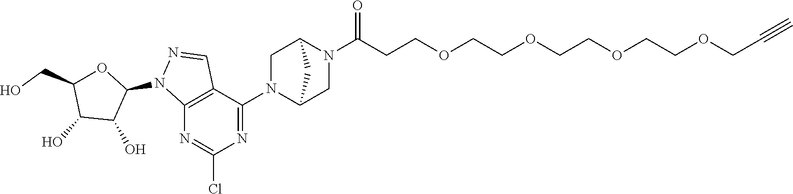 [SMILES: C#CCOCCOCCOCCOCCC(=O)N1C[C@@H]2C[C@H]1CN2c1nc(Cl)nc2c1cnn2[C@@H]1O[C@H](CO)[C@@H](O)[C@H]1O]

To the reaction solution from step c was added K₂CO₃ (414.6 mg, 3 mmol) and MeOH (3 ml). The resulting mixture was stirred at room temperature for 2 hours. The salt was filtered off and washed with acetonitrile. The filtrate was treated with Dowex 50× hydrogen form (1 g) and stirred for 5 minutes. The Dowex resin was then filtered and washed with acetonitrile. The filtrate was concentrated by rotary evaporation and carried to the subsequent step without purification. Ions found by LCMS: [M+Na]⁺=647.2, [M+H]⁺=625.2, [(M+2H)/2]⁺=313.2.

### Step e

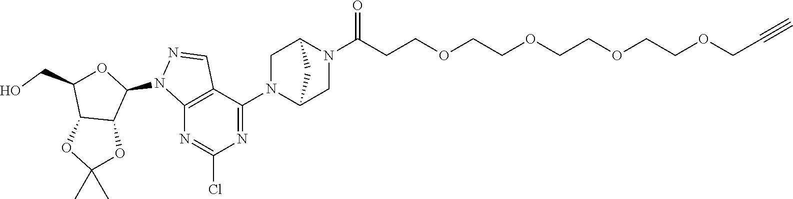 [SMILES: C#CCOCCOCCOCCOCCC(=O)N1C[C@@H]2C[C@H]1CN2c1nc(Cl)nc2c1cnn2[C@@H]1O[C@H](CO)[C@H]2OC(C)(C)O[C@H]21]

To the crude product from step d in anhydrous DMF (0.5 ml) was added 2,2-dimethoxypropane (0.3 ml), acetone (1 ml) and pTSA monohydrate (33 mg, 0.173 mmol). The resulting mixture was stirred at room temperature for 1 hour, then added with DIPEA (129.2 mg, 1 mmol) and directly purified by RPLC (100 g column, 5% to 100% acetonitrile and water). Yield 193.6 mg, 72.8% over three steps. Ions found by LCMS: [M+Na]⁺=687.2, [M+H]⁺=665.2.

### Step f

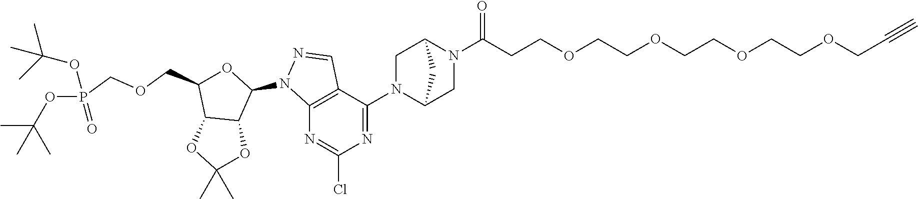 [SMILES: C#CCOCCOCCOCCOCCC(=O)N1C[C@@H]2C[C@H]1CN2c1nc(Cl)nc2c1cnn2[C@@H]1O[C@H](COCP(=O)(OC(C)(C)C)OC(C)(C)C)[C@H]2OC(C)(C)O[C@H]21]

A flame-dried reaction flask was filled with nitrogen and charged with product from step e (87.8 mg, 0.132 mmol) and anhydrous DMF (0.5 ml). After the solution was cooled in an ice-water bath, methanesulfonic acid, 1,1,1-trifluoro-, [bis(1,1-dimethylethoxy)phosphinyl]methyl ester (94 mg, 0.264 mmol) was added followed by sodium hydride, 60% dispersion in mineral (15.8 mg, 0.40 mmol). The reaction mixture was stirred at ˜5° C. for 2 hours, then carried to the subsequent step without purification. Ions found by LCMS: [M+Na]⁺=893.2, [M−tbu]⁺=815.2, [M−2tbu]⁺=759.2, [(M−2tBu)/2]⁺=380.2.

### Step g

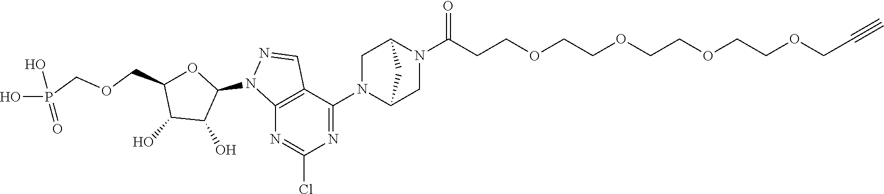 [SMILES: C#CCOCCOCCOCCOCCC(=O)N1C[C@@H]2C[C@H]1CN2c1nc(Cl)nc2c1cnn2[C@@H]1O[C@H](COCP(=O)(O)O)[C@@H](O)[C@H]1O]

The crude product from step f was slowly mixed with a mixture of TFA (1 ml) and water (0.2 ml). The ice-water bath was removed, and the resulting mixture was stirred for 20 hours. It was then directly purified through HPLC (5 to 50% acetonitrile and water, using 0.1% TFA as modifier). Yield 36.2 mg, 38.1%. Ions found by LCMS: [M+Na]⁺=741.2, [M+H]⁺=719.2, [(M+2H)/2]⁺=360.2.

### Step h

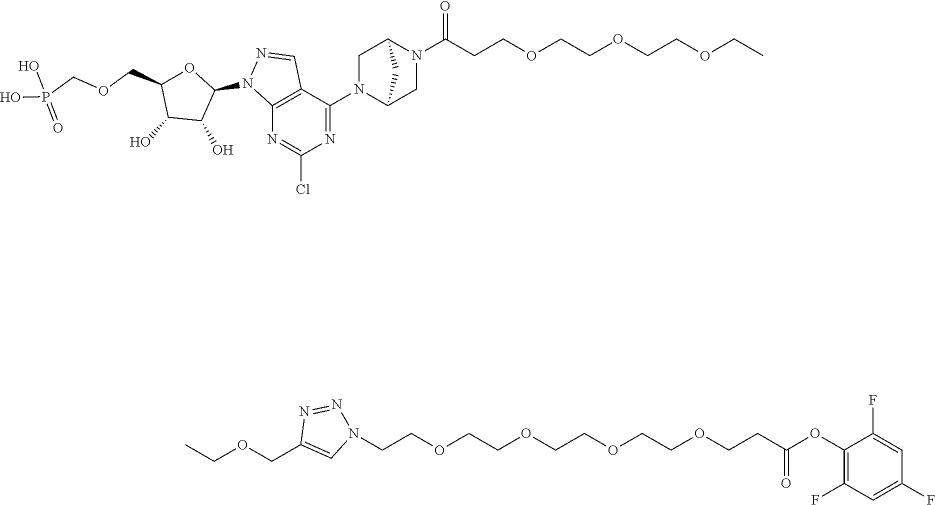

A solution of azido-PEG4-trifluorophenyl ester (19.6 mg, 0.0465 mmol) in anhydrous DMF (0.4 ml) was cooled in an ice-water bath and added with a pre-mixed THPTA (15.7 mg, 0.037 mmol) and Cu₂SO₄ (5.8 mg, 0.037 mmol) in water (0.2 ml). After stirred for 5 minutes, the mixture was added into product from step g (22.2 mg, 0.0309 mmol), followed by sodium ascorbate (19.8 mg, 0.1 mmol). The reaction was stirred for 30 minutes, then directly purified by HPLC (5 to 70% acetonitrile and water, using 0.1% TFA as modifier). Yield 19.6 mg, 55.6%. Ions found by LCMS: [M+H]⁺=1140.2, [(M+2H)/2]⁺=570.8.

### Synthesis of Conjugate 202

To a solution of SEQ ID NO: 80 (8.55 ml, 110 mg, 0.00193 mmol) in PBS 7.4 was added DMF (1.7 ml) and Int-335 (17.6 mg, 0.01543 mmol) in DMF (0.2 ml). Additional DMF (0.2 ml×5) were used to wash the glassware and combined to the reaction mixture. The pH of the reaction mixture was adjusted to 8.5 by 1.0 M borate buffer solution pH=8.5 (0.2 ml). After the reaction was gently rotate for 2 hours, a premixed solution (4 ml) of 150 mM histidine, 100 mM NH₄OH and 70 mM HCl (pH 8.5) was then added, and the resulting mixture was gently rotated for 1 hour. It was purified by dialysis and SEC. Maldi TOF analysis of the purified final product gave an average mass of 59,995 Da (DAR=4.8). Yield: 61.6 mg, 56.0%.

### Synthesis of Int-325

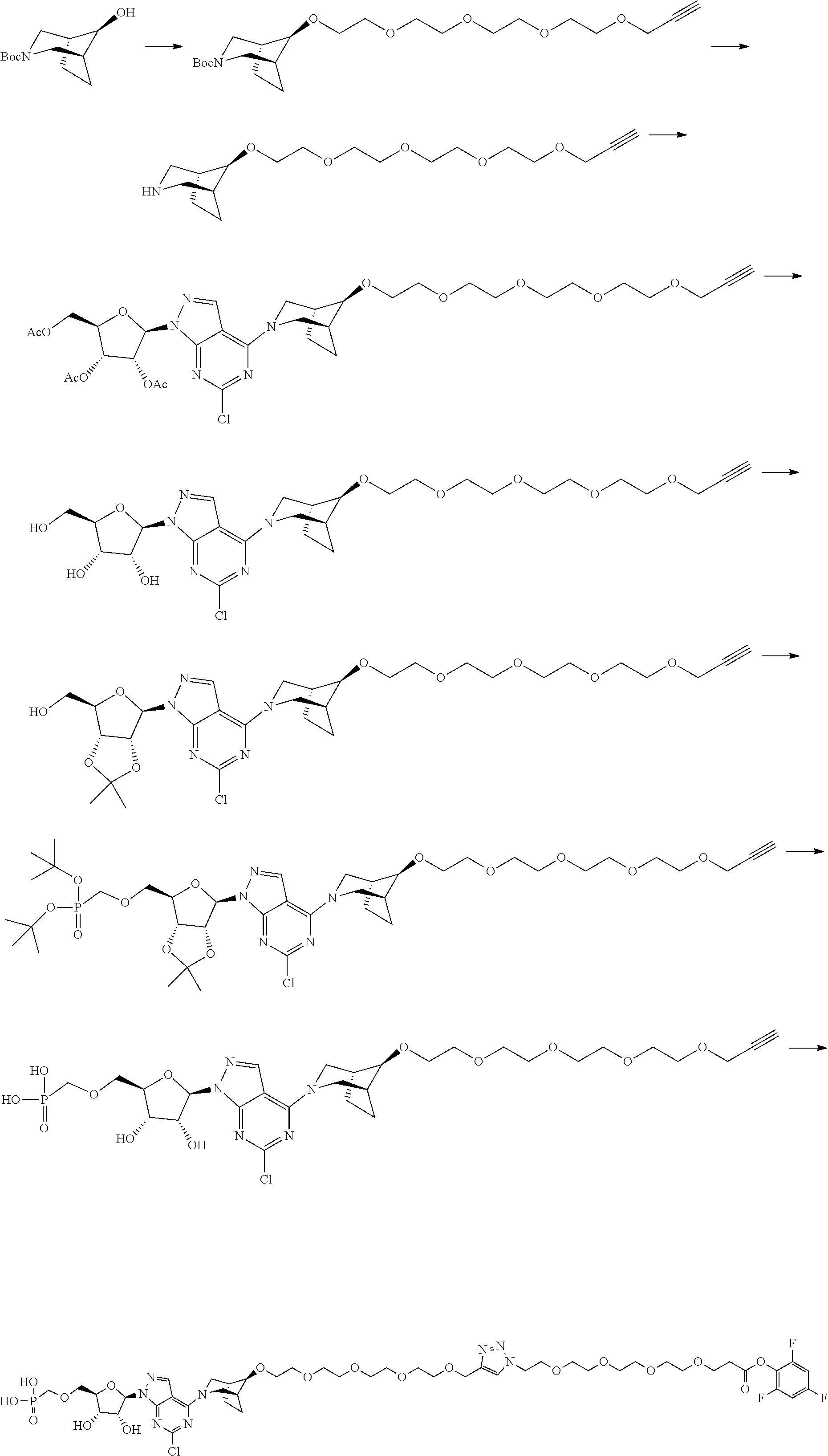

### Step a

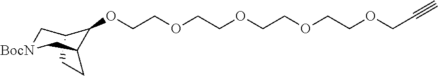 [SMILES: C#CCOCCOCCOCCOCCO[C@@H]1[C@@H]2CC[C@H]1CN(C(=O)OC(C)(C)C)C2]

A flame dried reaction flask was filled with nitrogen and charged with endo-tert-butyl 8-hydroxy-3-azabicyclo[3.2.1]octane-3-carboxylate (500 mg, 2.2 mmol) and anhydrous DMF (1.5 ml). The solution was slowly mixed with sodium hydride 50% dispersion in mineral oil (132 mg, 3.3 mmol). After stirring for 10 minutes, the reaction mixture was treated with propargyl-PEG4-mesyl ester (1.02 g, 3.3 mmol) and stirred at room temperature for 5 hours. Excess NaH was slowly quenched with water, and the reaction mixture was directly purified by RPLC (100 g column, 5 to 100% acetonitrile and water, using 0.1% TFA as modifier). Yield 763 mg, 78.5%. Ions found by LCMS: [M+Na]⁺=464.2, [M−tBu+H]⁺=386.2.

### Step b

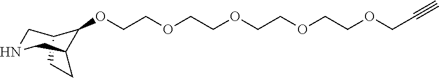 [SMILES: C#CCOCCOCCOCCOCCO[C@@H]1[C@@H]2CC[C@H]1CNC2]

Product from step a (763 mg, 1.728 mmol) was dissolved in TFA (1 ml), and the resulting solution was stirred at room temperature for 1 hour. It was then concentrated by rotary evaporation and further dried under high vacuum. The product was carried to the next step without purification. Ion found by LCMS: [M+H]⁺=341.2.

### Step c

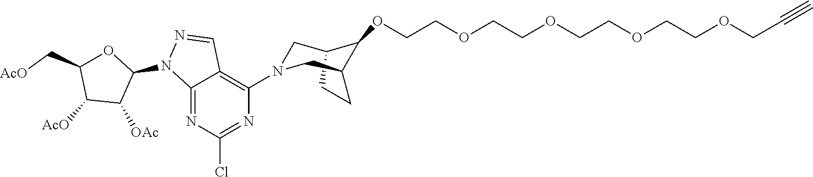 [SMILES: C#CCOCCOCCOCCOCCO[C@@H]1[C@@H]2CC[C@H]1CN(c1nc(Cl)nc3c1cnn3[C@@H]1O[C@H](COC(C)=O)[C@@H](OC(C)=O)[C@H]1OC(C)=O)C2]

To a solution of Intermediate B (224 mg, 0.5 mmol) in anhydrous DMF (0.4 ml) was added product from step b (273.3 mg, 0.6 mmol), DMF (1 ml) and DIPEA (387.7 mg, 3.0 mmol). The reaction mixture was stirred at room temperature for 1 hour and then extracted with water (3 ml) and DCM (5 ml×2). The combined organic layers were concentrated by rotary evaporation and carried to subsequent step without further purification. Ions found by LCMS: [M+H]⁺=752.2.

### Step d

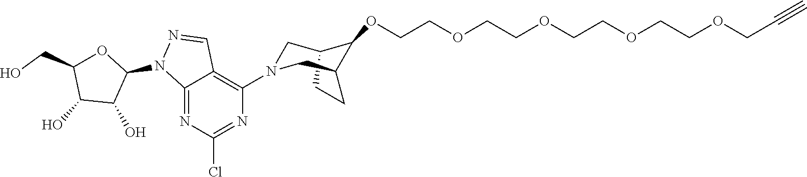 [SMILES: C#CCOCCOCCOCCOCCO[C@@H]1[C@@H]2CC[C@H]1CN(c1nc(Cl)nc3c1cnn3[C@@H]1O[C@H](CO)[C@@H](O)[C@H]1O)C2]

To the reaction solution from step c was added K₂CO₃ (414.6 mg, 3 mmol) and MeOH (5 ml). The resulting mixture was stirred at room temperature for 2 hours. The salt was filtered off and washed with acetonitrile. The filtrate was treated with DOWEX 50× hydrogen form (1 g) and stirred for 5 minutes. DOWEX 50× hydrogen form was then filtered and washed with acetonitrile. The filtrate was concentrated by rotary evaporation and carried to the subsequent step without purification. Ions found by LCMS: [M+Na]⁺=648.2, [M+H]⁺=626.2.

### Step e

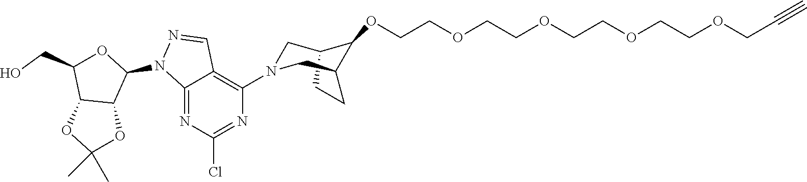 [SMILES: C#CCOCCOCCOCCOCCO[C@@H]1[C@@H]2CC[C@H]1CN(c1nc(Cl)nc3c1cnn3[C@@H]1O[C@H](CO)[C@H]3OC(C)(C)O[C@H]31)C2]

To the reaction solution from step d was added 2,2-dimethoxypropane (0.3 ml), acetone (1 ml) and pTSA monohydrate (285 mg, 1.5 mmol). The resulting mixture was stirred at room temperature for 1 hour, then added with DIPEA (258.5 mg, 2 mmol) and directly purified by RPLC (100 g column, 5 to 100% acetonitrile and water). Yield 298.5 mg, 89.6% over three steps. Ion found by LCMS: [M+H]⁺=666.2.

### Step f

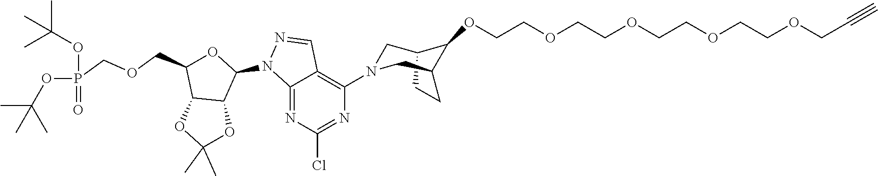 [SMILES: C#CCOCCOCCOCCOCCO[C@@H]1[C@@H]2CC[C@H]1CN(c1nc(Cl)nc3c1cnn3[C@@H]1O[C@H](COCP(=O)(OC(C)(C)C)OC(C)(C)C)[C@H]3OC(C)(C)O[C@H]31)C2]

A flame-dried reaction flask was filled with nitrogen and charged with product from step e (298.5 mg, 0.448 mmol) and anhydrous DMF (1 ml). After the solution was cooled in an ice-water bath, sodium hydride, 60% dispersion in mineral (53.8 mg, 1.34 mmol) was added, followed by methanesulfonic acid, 1,1,1-trifluoro-, [bis(1,1-dimethylethoxy)phosphinyl]methyl ester (365 mg, 1.0 mmol) and anhydrous DMF (0.5 ml). The reaction mixture was stirred at ˜5° C. for 1 hour, then quenched with water (5 ml) and extracted with EtOAc (10 ml). The organic layers were concentrated by rotary evaporation and carried to the subsequent step without further purification. Ions found by LCMS: [M+Na]⁺=894.4, [M−tbu]⁺=816.2, [M−2tbu]⁺=760.2.

### Step g

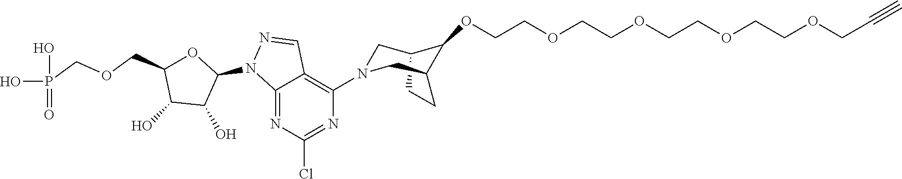 [SMILES: C#CCOCCOCCOCCOCCO[C@@H]1[C@@H]2CC[C@H]1CN(c1nc(Cl)nc3c1cnn3[C@@H]1O[C@H](COCP(=O)(O)O)[C@@H](O)[C@H]1O)C2]

The crude product from step f was cooled in an ice-water bath and slowly added with a pre-cooled mixture of TFA (2 ml) and water (0.5 ml). The ice-water bath was removed, and the resulting mixture was stirred for 20 hours. It was then directly purified through HPLC (5 to 50% acetonitrile and water, using 0.1% TFA as modifier). Yield 170 mg, 52.7% over two steps. Ions found by LCMS: [M+Na]⁺=742.2, [M+H]⁺=720.2.

### Step h

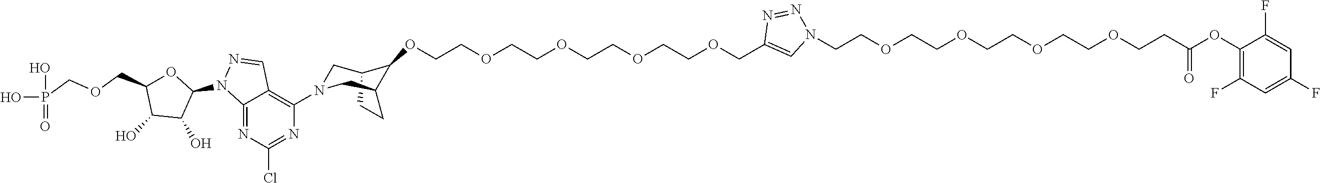 [SMILES: O=C(CCOCCOCCOCCOCCn1cc(COCCOCCOCCOCCO[C@@H]2[C@@H]3CC[C@H]2CN(c2nc(Cl)nc4c2cnn4[C@@H]2O[C@H](COCP(=O)(O)O)[C@@H](O)[C@H]2O)C3)nn1)Oc1c(F)cc(F)cc1F]

A solution of azido-PEG4-trifluorophenyl ester (15.6 mg, 0.0362 mmol) in anhydrous DMF (0.4 ml) was treated with a premixed solution of THPTA (15.7 mg, 0.0362 mmol), Cu₂SO₄ (5.8 mg, 0.0362 mmol) in water (0.2 ml). After stirred for 5 minutes, the mixture was treated with product from step g (21.7 mg, 0.0301 mmol), followed by DMF (0.2 ml) and sodium ascorbate (19.8 mg, 0.1 mmol). The reaction was stirred for one hour, then directly purified by HPLC (5 to 70% acetonitrile and water, using 0.1% TFA as modifier). Yield 21.3 mg, 62.0%. Ions found by LCMS: [(M+2H)/2]⁺=571.2.

### Synthesis of Conjugate 192

Conjugate 192 was prepared analogously to Conjugate 202, where Int-335 was replaced with Int-325. Maldi TOF analysis of the purified final product gave an average mass of 62,910 Da (DAR=7.7). Yield: 70.4 mg, 51.4%.

### Synthesis of Int-326

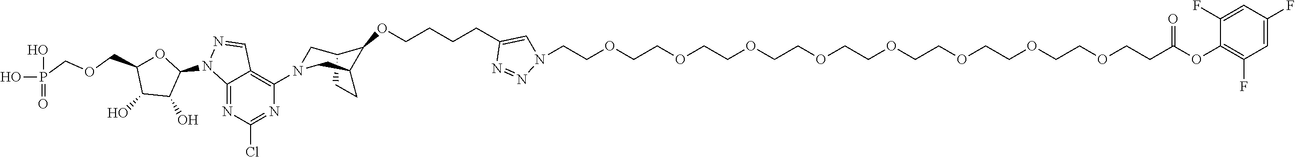 [SMILES: O=C(CCOCCOCCOCCOCCOCCOCCOCCOCCn1cc(CCCCO[C@@H]2[C@@H]3CC[C@H]2CN(c2nc(Cl)nc4c2cnn4[C@@H]2O[C@H](COCP(=O)(O)O)[C@@H](O)[C@H]2O)C3)nn1)Oc1c(F)cc(F)cc1F]

The title compound was prepared analogously to Int-325, where propargyl-PEG4-mesyl ester was replaced with hex-5-ynyl methanesulfonate. [(M+Na)/2]⁺=603.4, [(M+2H)/2]⁺=592.4.

### Synthesis of Conjugate 193

Conjugate 193 was prepared analogously to Conjugate 192, where Int-325 was replaced with Int-326. Maldi TOF analysis of the purified final product gave an average mass of 64,073 Da (DAR=8.5). Yield: 64.0 mg, 53.3%.

### Synthesis of Int-327

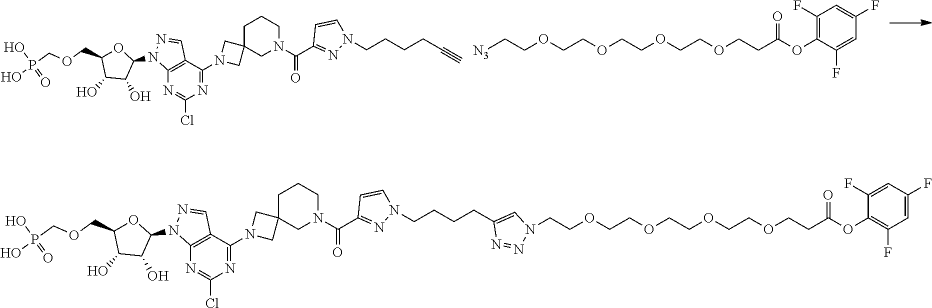

The step c product described in the Int-305 synthesis (50 mg, 0.07 mmol) and azido-PEG4-trifluorophenyl ester described in Int-291 (37 mg, 0.08 mmol) were dissolved in DMF (0.6 mL) and cooled to 0° C. This mixture was treated with a pre-mixed solution of a solution of copper(II) sulfate (5.8 mg, 0.03 mmol), sodium ascorbate (43 mg, 0.22 mmol), and BTTAA (15 mg, 0.03 mmol) in water (0.6 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a colorless oil. Yield 73 mg, 65%. Ion(s) found by LCMS [(M+2H)/2]⁺=551.2.

### Synthesis of Conjugate 194

Int-327 (53 mg, 0.02 mmol) in DMF (0.4 mL) was added to SEQ ID NO: 116 (133 mg in 4.3 mL PBS 7.4, 0.002 mmol) in PBS at pH 7.4 as described in the general conjugation procedure. Maldi TOF analysis of the purified final product gave an average mass of 61,017 Da. (DAR=6.2). Yield 86.6 mg, 65.1%.

### Synthesis of Int-328

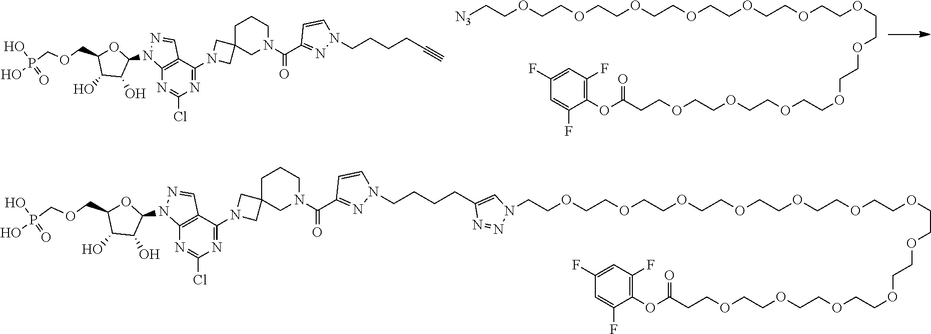 [SMILES: C#CCCCCn1ccc(C(=O)N2CCCC3(C2)CN(c2nc(Cl)nc4c2cnn4[C@@H]2O[C@H](COCP(=O)(O)O)[C@@H](O)[C@H]2O)C3)n1 ; [N-]=[N+]=NCCOCCOCCOCCOCCOCCOCCOCCOCCOCCOCCOCCOCCC(=O)Oc1c(F)cc(F)cc1F ; O=C(CCOCCOCCOCCOCCOCCOCCOCCOCCOCCOCCOCCOCCn1cc(CCCCn2ccc(C(=O)N3CCCC4(C3)CN(c3nc(Cl)nc5c3cnn5[C@@H]3O[C@H](COCP(=O)(O)O)[C@@H](O)[C@H]3O)C4)n2)nn1)Oc1c(F)cc(F)cc1F]

The step c product described in the Int-305 synthesis (50 mg, 0.07 mmol) and azido-PEG12-trifluorophenyl ester described in step a of the Int-284 synthesis (68 mg, 0.08 mmol) were dissolved in DMF (0.6 mL) and cooled to 0° C. The mixture was treated with a pre-mixed solution of a solution of copper(II) sulfate (5.8 mg, 0.03 mmol), sodium ascorbate (43 mg, 0.22 mmol), and BTTAA (15 mg, 0.03 mmol) in water (0.6 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a colorless oil. Yield 77 mg, 71%. Ion(s) found by LCMS [(M+2H)/2]⁺=727.4.

### Synthesis of Conjugate 195

Int-328 (77 mg, 0.05 mmol) in DMF (0.4 mL) was added to SEQ ID NO: 116 (146 mg in 4.8 mL PBS 7.4, 0.002 mmol) in PBS at pH 7.4 as described in the general conjugation procedure. Maldi TOF analysis of the purified final product gave an average mass of 64,810 Da. (DAR=7.4). Yield 100 mg, 68.9%.

### Synthesis of Int-329

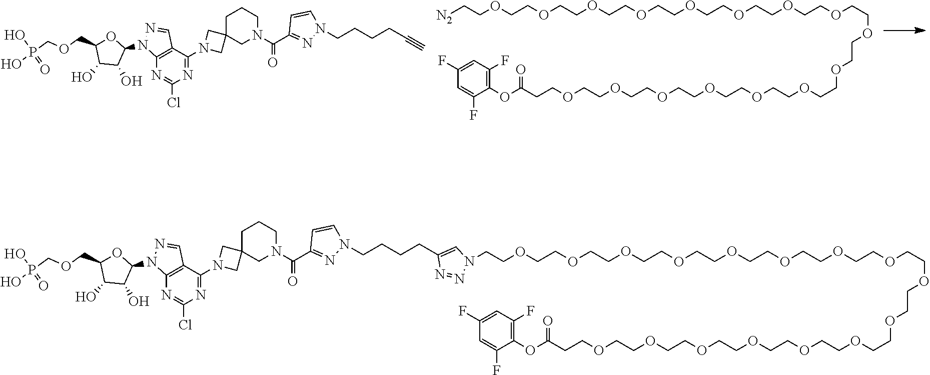

The step c product described in the Int-305 synthesis (50 mg, 0.07 mmol) and azido-PEG16-trifluorophenyl ester described in step a of the Int-303 synthesis (83 mg, 0.08 mmol) were dissolved in DMF (0.6 mL) and cooled to 0° C. The mixture was treated with a pre-mixed solution of a solution of copper(II) sulfate (5.8 mg, 0.03 mmol), sodium ascorbate (43 mg, 0.22 mmol), and BTTAA (15 mg, 0.03 mmol) in water (0.6 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a colorless oil. Yield 88 mg, 73%. Ion(s) found by LCMS [(M+2H)/2]⁺=815.5.

### Synthesis of Conjugate 196

Int-329 (88 mg, 0.05 mmol) in DMF (0.4 mL) was added to SEQ ID NO: 116 (149 mg in 4.9 mL PBS 7.4, 0.002 mmol) in PBS at pH 7.4 as described in the general conjugation procedure. Maldi TOF analysis of the purified final product gave an average mass of 65,434 Da. (DAR=7). Yield 99 mg, 66%.

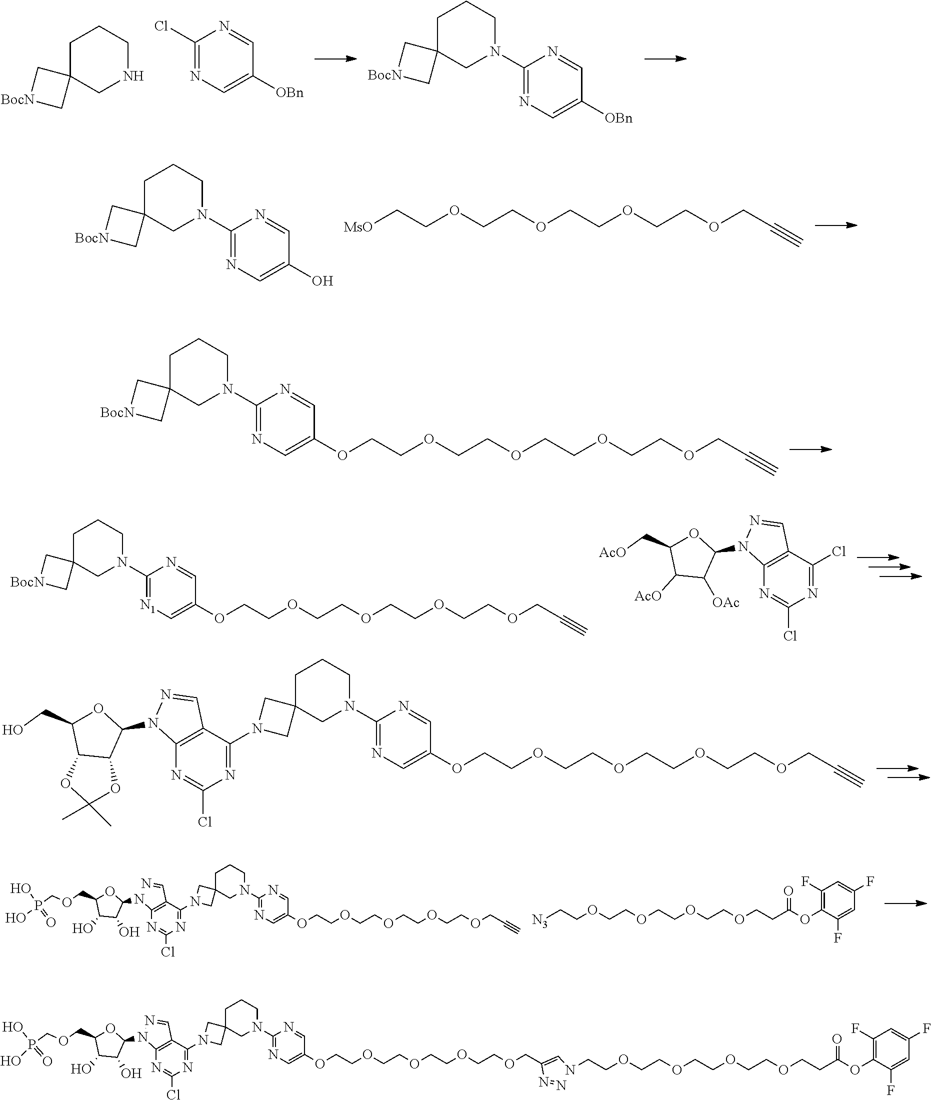

### Step a

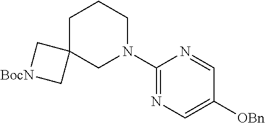 [SMILES: CC(C)(C)OC(=O)N1CC2(CCCN(c3ncc(OCc4ccccc4)cn3)C2)C1]

To a solution of Boc-azetidine spirocycle (0.45 g, 2 mmol) in toluene (40 ml) was added p-benzyloxy-bromopyrimidine (0.44 g, 2 mmol) Pd₂(dba)₃ (0.09 g, 0.10 mmol) and X-Phos (0.095 g, 0.20 mmol) at room temperature and heated to 50° C. for 15 minutes under nitrogen. Sodium t-butoxide (0.57 g, 6 mmol) was added to the reaction mixture and the reaction mixture was heated at 100° C. for 1 hr. On completion of reaction, the reaction mixture was filtered through celite bed and washed with ethyl acetate. The combined organic layers were washed with water, dried over anhydrous sodium sulfate and concentrated under reduced pressure. The resultant residue was purified by flash chromatography (Hexanes/EtOAc gradient) to yield product. Yield 0.65 g, 79%. Ion(s) found by LCMS: [M+H]⁺=411.5.

### Step b

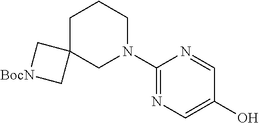 [SMILES: CC(C)(C)OC(=O)N1CC2(CCCN(c3ncc(O)cn3)C2)C1]

To a solution of step-a product (0.42 g, 1.0 mmol) in methanol (15 ml) was added Pd(OH)₂ (0.2 g). The reaction solution was stirred under an H₂ atmosphere overnight, filtered through a celite, washed with DCM and concentrated to give the crude which was used in the next step without purification. Yield 0.32 g, 94%. Ion(s) found by LCMS: [M+H]⁺=321.3.

### Step c

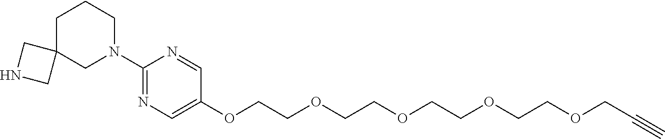 [SMILES: C#CCOCCOCCOCCOCCOc1cnc(N2CCCC3(CNC3)C2)nc1]

To a solution of step b product (0.32 g, 1.0 mmol) and propargyl-PEG4-mesylate (0.37 g, 1.2 mmol) in acetonitrile (5 mL) was added potassium carbonate (0.41 g, 3.0 mmol). The reaction solution at 70° C. was stirred at room temperature for 2 hours, filtered and concentrated. The residue was purified by semi-preparative HPLC (5% to 100% ACN/water, 0.1% TFA modifier). The product was a colorless oil. Yield 0.32 g, 60%. Ion(s) found by LCMS [M+H]⁺=535.6. Intermediate N-Boc derivative was dissolved in DCM (10 mL) and TFA (4 mL). The resulting mixture was stirred for 2 h at room temperature then solvent was removed under reduced pressure and dried under vacuum to yield the desired compound as its TFA salt. The product was a yellow oil. Ion(s) found by LCMS: [M+H]⁺=435.6.

### Step d

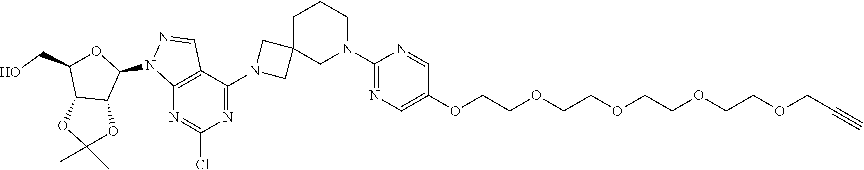 [SMILES: C#CCOCCOCCOCCOCCOc1cnc(N2CCCC3(C2)CN(c2nc(Cl)nc4c2cnn4[C@@H]2O[C@H](CO)[C@H]4OC(C)(C)O[C@H]42)C3)nc1]

A solution of Intermediate B (0.22 g, 0.49 mmol), spirocyclic azetidine from step c (0.24 mg, 0.45 mmol), and triethylamine (0.093 mL, 0.67 mmol) in anhydrous EtOH (0.5 mL) was stirred at room temperature for 30 min. The solution was concentrated, and the residue was dissolved in 7 M NH₃/MeOH (5 mL). The reaction was stirred at room temperature overnight and mixture was concentrated to dryness under reduced pressure, then dissolved in acetone (1.3 mL). 2,2-dimethoxypropane (1.3 mL, 10 mmol) and p-TsOH (0.1 g, 0.56 mmol) was added and the mixture was stirred overnight at room temperature for 2 hours. The reaction mixture was concentrated to dryness under reduced pressure and purified by flash chromatography (Hexanes/EtOAc) to yield product. The product was a white foam. Yield 0.25 mg, 73%. Ion(s) found by LCMS: [M+H]⁺=760.2.

### Step e

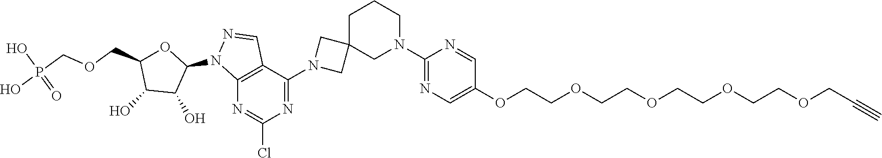 [SMILES: C#CCOCCOCCOCCOCCOc1cnc(N2CCCC3(C2)CN(c2nc(Cl)nc4c2cnn4[C@@H]2O[C@H](COCP(=O)(O)O)[C@@H](O)[C@H]2O)C3)nc1]

A solution of acetonide derivative from previous step (0.25 g, 0.32 mmol) and [bis(tert-butoxy)carbonyl]methyl (trifluoromethyl)sulfonate (0.35 g, 0.96 mmol) in dry DMF (3 mL) was cooled using ice-water bath. 60% NaH in mineral oil (0.07 g, 1.9 mmol) was added to this and stirred at room temperature for 2 h. The reaction mixture was quenched with saturated ammonium chloride and extracted with EtOAc. The organic phase was washed with brine and dried over sodium sulfate concentrated to give crude product which was used in the next step without purification. Crude product was dissolved in 50% aqueous TFA (3 mL) and stirred at room temperature for 12 h, concentrated and the residue was purified by semi-preparative HPLC (10-100% ACN: H₂O, 0.1% TFA modifier). The product was a white solid. Yield 0.14 g, 52%. Ion(s) found by LCMS: [M+H]⁺=814.1.

### Step f

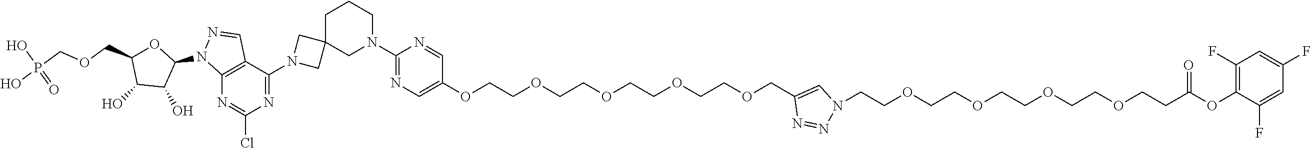 [SMILES: O=C(CCOCCOCCOCCOCCn1cc(COCCOCCOCCOCCOc2cnc(N3CCCC4(C3)CN(c3nc(Cl)nc5c3cnn5[C@@H]3O[C@H](COCP(=O)(O)O)[C@@H](O)[C@H]3O)C4)nc2)nn1)Oc1c(F)cc(F)cc1F]

To a solution of step e product (50 mg, 0.061 mmol) and propargyl-PEG4-trifluorophenyl ester (33 mg, 0.07 mmol) dissolved in DMF (0.6 mL) was cooled to 0° C., and treated with a pre-mixed solution of a solution of copper(II) sulfate (4 mg, 0.03 mmol), sodium ascorbate (36 mg, 0.18 mmol), and BTTAA (13 mg, 0.03 mmol) in water (0.6 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). Yield 52 mg, 68%. Ion(s) found by LCMS [(M+2H)/2]⁺=618.2.

### Synthesis of Conjugate 197

Int-330 (32 mg, 0.026 mmol) in DMF (0.5 mL) was added to SEQ ID NO: 116 (120 mg, 0.002 mmol) in 6.7 mL PBS at pH 7.4 as described in the general conjugation procedure. Maldi TOF analysis of the purified final product gave an average mass of 64,085 Da. (DAR=8.1). Yield 86 mg, 72%.

### Synthesis of Int-331

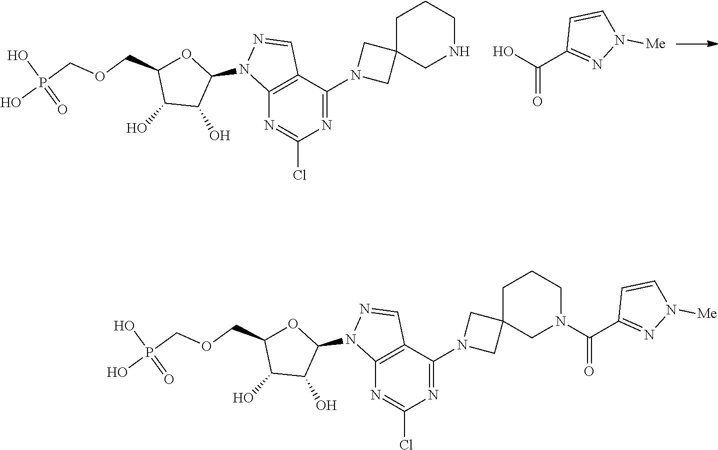 [SMILES: O=P(O)(O)COC[C@H]1O[C@@H](n2ncc3c(N4CC5(CCCNC5)C4)nc(Cl)nc32)[C@H](O)[C@@H]1O ; Cn1ccc(C(=O)O)n1 ; Cn1ccc(C(=O)N2CCCC3(C2)CN(c2nc(Cl)nc4c2cnn4[C@@H]2O[C@H](COCP(=O)(O)O)[C@@H](O)[C@H]2O)C3)n1]

To a solution of Int-287 (10 mg, 0.02 mmol) and pyrazole-acid (3.7 mg, 0.02 mmol) in DMF was added DIEA (0.02 mL, 0.11 mmol) and HATU (15 mg, 0.04 mmol). The reaction solution was stirred at room temperature for 30 min. The reaction was concentrated and the residue was purified by semi-preparative HPLC (5% to 100% ACN/water, 0.1% TFA modifier) to give the product as an off-white foam. Yield 6.5 mg, 53%. Ion(s) found by LCMS: [M+H]⁺=613.9.

### Synthesis of Int-332

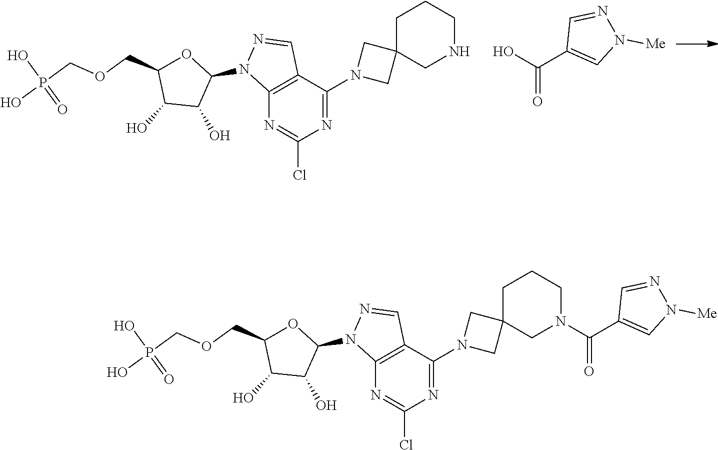 [SMILES: O=P(O)(O)COC[C@H]1O[C@@H](n2ncc3c(N4CC5(CCCNC5)C4)nc(Cl)nc32)[C@H](O)[C@@H]1O ; Cn1cc(C(=O)O)cn1 ; Cn1cc(C(=O)N2CCCC3(C2)CN(c2nc(Cl)nc4c2cnn4[C@@H]2O[C@H](COCP(=O)(O)O)[C@@H](O)[C@H]2O)C3)cn1]

To a solution of Int-287 (10 mg, 0.02 mmol) and pyrazole-acid (3.7 mg, 0.02 mmol) in DMF was added DIEA (0.02 mL, 0.11 mmol) and HATU (15 mg, 0.04 mmol). The reaction solution was stirred at room temperature for 30 min. The reaction was concentrated and the residue was purified by semi-preparative HPLC (5% to 100% ACN/water, 0.1% TFA modifier) to give the product as an off-white foam. Yield 8 mg, 65%. Ion(s) found by LCMS: [M+H]⁺=613.9.

### Synthesis of Int-333

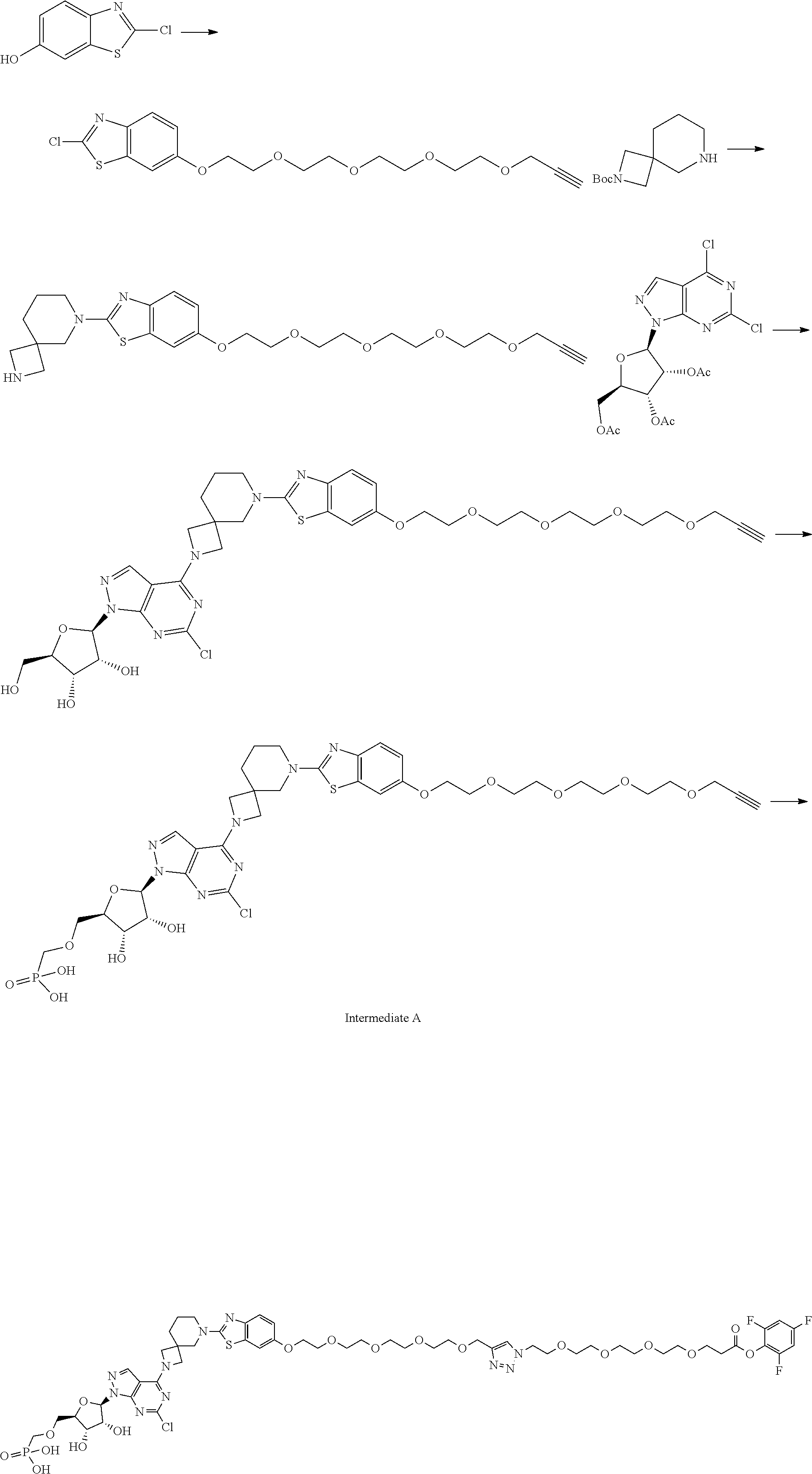

### Step a

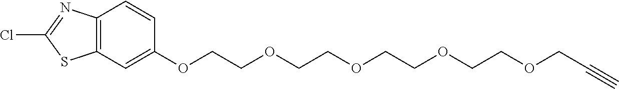 [SMILES: C#CCOCCOCCOCCOCCOc1ccc2nc(Cl)sc2c1]

To a solution of 2-chlorobenzothiazol-6-ol (371 mg, 2 mmol) in ACN (5 mL) was added cesium carbonate (1.30 g, 4.00 mmol) and propargyl-PEG₄-mesylate (744 mg, 2.4 mmol). The resulting mixture was stirred at 80° C. for 4 hours. The reaction mixture was cooled to room temperature and solvent was removed under reduced pressure. The residue was partitioned with EtOAc (25 mL) and water (25 mL). The organic layer was collected, and the aqueous phase was extracted with EtOAc (2×). The combined organic extracts were washed with water, brine and dried over dried over sodium sulfate and concentrated in vacuo to get the crude product, which was purified by RPLC (10% to 100% ACN/water, 0.1% TFA modifier). The product was a reddish viscous liquid. Yield 800 mg, 76%. Ion (s) found by LCMS: [M+H]⁺=400.1.

### Step b

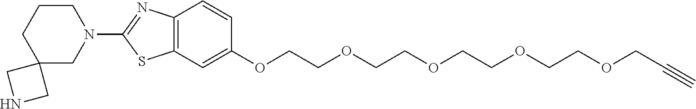 [SMILES: C#CCOCCOCCOCCOCCOc1ccc2nc(N3CCCC4(CNC4)C3)sc2c1]

The product from step a (600 mg, 1.5 mmol), tert-butyl 2,6-diazaspiro[3.5]nonane-2-carboxylate (340 mg, 1.5 mmol) and Hunigs base (1.3 mL, 7.5 mmol) were dissolved in DMF (5 mL) and heated at 120° C. for 12 hours. The crude reaction mixture was cooled to room temperature and solvent was evaporated. The crude residue was purified directly via RPLC (10% to 100% ACN/water, 0.1% TFA modifier). The pure fractions were pooled and lyophilized to afford the Boc protected amine intermediate as a reddish viscous liquid. Yield 662 mg, 75%. Ion found by LCMS: [M+H]⁺=590.2. Intermediate N-Boc derivative (662 mg, 1.12 mmol) was dissolved in DCM (5 mL) and TFA (3 mL). The resulting mixture was stirred for 2 hours at room temperature then solvent was removed under reduced pressure and dried under vacuum to yield the compound as its TFA salt. The product was a red viscous liquid. Yield 950 mg, (assumed quantitative). Ion (s) found by LCMS: [M+H]⁺=490.2.

### Step c

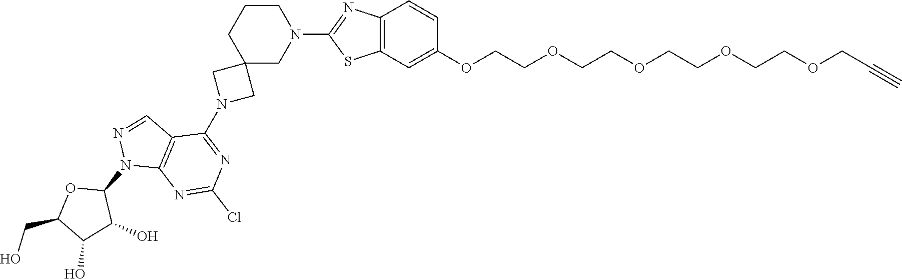 [SMILES: C#CCOCCOCCOCCOCCOc1ccc2nc(N3CCCC4(C3)CN(c3nc(Cl)nc5c3cnn5[C@@H]3O[C@H](CO)[C@@H](O)[C@H]3O)C4)sc2c1]

A mixture of intermediate B (300 mg, 0.67 mmol), the azetidine derivative from previous step (394 mg, 0.8 mmol), and DIEA (0.19 mL, 1.34 mmol) in ethanol (6 mL, 1.12 mmol) were heated at 50° C. for 1 hour. The mixture was cooled to ambient temperature and concentrated on a rotary evaporator. The crude residue was dissolved in ethyl acetate (50 mL) and washed with water and brine. The organic layer was dried over sodium sulfate, filtered, and concentrated to afford the triacetate intermediate as an off-white foam. Ion(s) found by LCMS: [M+H]⁺=900.2. The crude triacetate was dissolved in methanol (5 mL). Potassium carbonate (325 mg, 2.35 mmol) was added to the solution and the mixture was stirred at ambient temperature for 2 hours. The mixture was concentrated under reduced pressure The crude residue was dissolved in ethyl acetate (50 mL) and washed with water and brine. The organic layer was dried over sodium sulfate, filtered, and concentrated to afford the crude product which was purified by RPLC (10% to 100% ACN/water, 0.1% TFA modifier) to yield the title compound as an off-white foam. Yield 519 mg, 52%. Ion (s) found by LCMS: [M+H]⁺=774.2.

### Step d

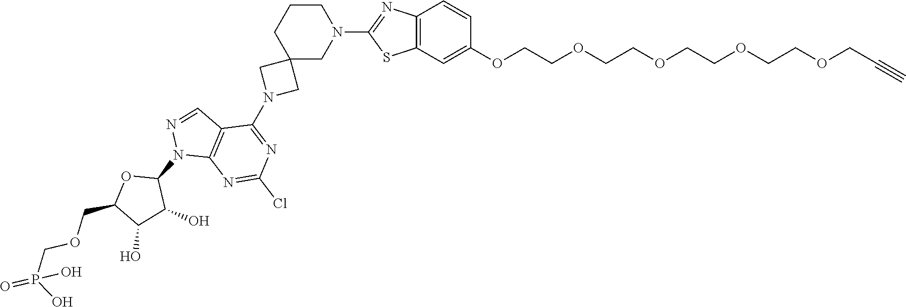 [SMILES: C#CCOCCOCCOCCOCCOc1ccc2nc(N3CCCC4(C3)CN(c3nc(Cl)nc5c3cnn5[C@@H]3O[C@H](COCP(=O)(O)O)[C@@H](O)[C@H]3O)C4)sc2c1]

To a solution of the product from previous step (270 mg, 0.35 mmol) and 2,2-dimethoxypropane (109 mg, 1.05 mmol) in acetone (10 mL) at room temperature was added p-toluene sulfonic acid (60 mg, 0.35 mmol). The reaction was stirred for two hours then concentrated under reduced pressure. The crude residue was re-dissolved in EtOAc (20 mL) and washed with saturated NaHCO₃. The organic layer was dried over sodium sulfate, filtered, and concentrated in vacuo to provide acetonide derivative as an off-white solid, which was used for next step without further purification. Ion (s) found by LCMS: [M+H]⁺=814.2. A solution of acetonide derivative and [bis(tert-butoxy)carbonyl]methyl (trifluoromethyl)sulfonate (372 mg, 1.05 mmol) in dry DMF (3 mL) was cooled using ice-water bath. 60% NaH in mineral oil (84 mg, 2.1 mmol) was added to this and gradually warmed to room temperature and stirred for 1 h. The reaction mixture was quenched with saturated ammonium chloride then extracted with EtOAc. The organic phase was washed with brine, dried over sodium sulfate, and concentrated to give crude product which was used in the next step without purification. Ion(s) found by LCMS: [M+H]⁺=908.2. Crude product was dissolved in 50% aqueous TFA (10 mL) and stirred at room temperature for 2 hours, concentrated, and purified by semi-preparative HPLC (10% to 100% ACN/water, 0.1% TFA modifier). The product was a white solid. Yield 116 mg, 38.3%. Ion(s) found by LCMS: [M+H]⁺=868.2.

### Step e

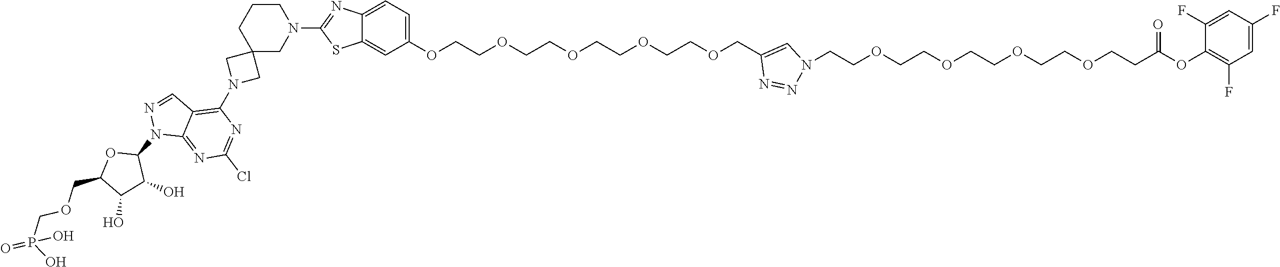 [SMILES: O=C(CCOCCOCCOCCOCCn1cc(COCCOCCOCCOCCOc2ccc3nc(N4CCCC5(C4)CN(c4nc(Cl)nc6c4cnn6[C@@H]4O[C@H](COCP(=O)(O)O)[C@@H](O)[C@H]4O)C5)sc3c2)nn1)Oc1c(F)cc(F)cc1F]

A solution of product from the previous step (50 mg, 0.058 mmol) and intermediate A (24 mg, 0.058 mmol, prepared as described in Example ??), dissolved in DMF:H₂O (1:3, 1.5 mL) were cooled to 0° C., then treated with a pre-mixed solution of a solution of copper(II) sulfate (1 mg, 0.005 mmol), sodium ascorbate (34 mg, 0.172 mmol), and BTTA (5 mg, 0.05 mmol) dissolved in water (0.5 mL). The resulting solution was stirred for 5 minutes at the same temperature and gradually warmed to room temperature and stirred for an additional 15 minutes. When the reaction was complete (by HPLC), the mixture was acidified with a few drops of glacial acetic acid and 125 mM EDTA (pH 6) and the product was purified by reversed phase HPLC (10% to 100% ACN/water, 0.1% TFA modifier). The product was a white solid. Yield 45 mg, 60.1%. Ion(s) found by LCMS [(M+2H)/2]⁺=645.2.

### Synthesis of Conjugate 198

Int-333 (35 mg, 0.027 mmol) in DMF (0.5 mL) was added to SEQ ID NO: 80 (150 mg, 11.5 mL in PBS at pH 8.4) as described in the general conjugation procedure. Maldi TOF analysis of the purified final product gave an average mass of 65,142 Da. (DAR=8.7). Yield 90 mg, 60%.

### Synthesis of Int-334

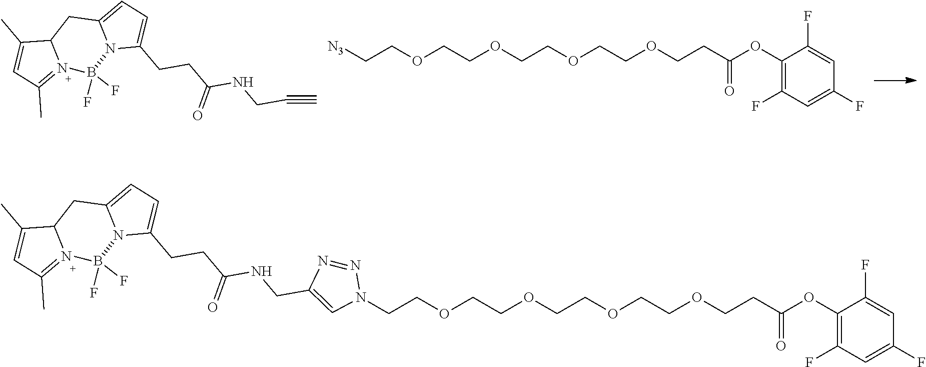

BDP-alkyne (25 mg, 0.07 mmol) and azido-PEG4-trifluorophenyl ester which was prepared as described in Int-291 synthesis (41 mg, 0.09 mmol) was dissolved in DMF (0.5 mL). The solution was cooled to 0° C., and then treated with a pre-mixed solution of a solution of copper(II) sulfate (6 mg, 0.03 mmol), sodium ascorbate (45 mg, 0.22 mmol), and BTTAA (16 mg, 0.03 mmol) water (0.5 mL) then stirred for 5 minutes at the same temperature, and then gradually warmed to room temperature and stirred for 15 minutes. When the reaction was complete (by HPLC), the product was purified by reversed phase HPLC (10% to 100% ACN/water, 0.1% TFA modifier). The product was a colorless oil. Yield 42 mg, 73%. Ion(s) found by LCMS [M+H]⁺=752.5.

### Synthesis of Conjugate 199

BODIPY trifluorophenol ester (0.4 mg, 0.0006 mmol) (described in Int-334) and Int-305 (8 mg, 0.006 mmol) in DMF (0.2 mL) were added to SEQ ID NO: 116 (30 mg, 0.0005 mmol) in 1.6 mL of PBS at pH 7.4 as described in the general conjugation procedure. Maldi TOF analysis of the purified final product gave an average mass of 66,036 Da. (DAR=9.6). Yield 23 mg, 78%.

### Synthesis of Conjugate 200

Int-334 (0.4 mg, 0.0006 mmol) and Int-305 (8 mg, 0.006 mmol) in DMF (0.2 mL) was added to SEQ ID NO: 116 (30 mg in 1.6 mL PBS 7.4, 0.0005 mmol) in PBS at pH 7.4 as described in the general conjugation procedure. Maldi TOF analysis of the purified final product gave an average mass of 66,036 Da. (DAR=9.6). Yield 23 mg, 78%.

### Synthesis of Conjugate 201

Int-334 (0.8 mg, 0.0009 mmol) and Int-305 (8 mg, 0.006 mmol) in DMF (0.2 mL) was added to SEQ ID NO: 116 (30 mg in 1.6 mL PBS 7.4, 0.0005 mmol) in PBS at pH 7.4 as described in the general conjugation procedure. Maldi TOF analysis of the purified final product gave an average mass of 65,796 Da. (DAR=9.4). Yield 23 mg, 79%.

### Biological Assays

### Cell-Free CD73 Enzyme Inhibition Assay: Inhibition of Purified Recombinant Human CD73

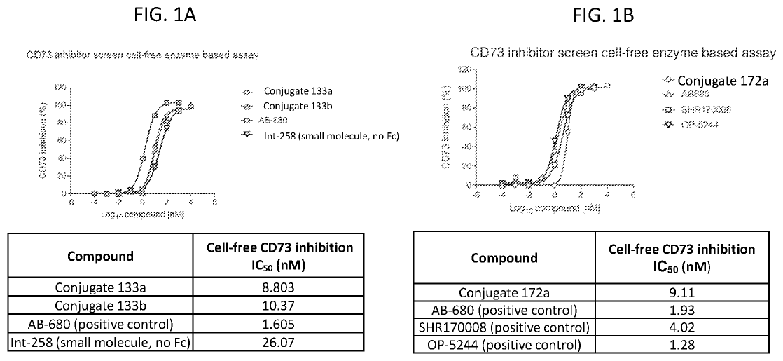

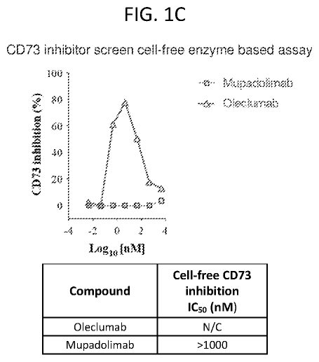

The cell-free CD73 enzyme inhibition assay was performed in a 96-well plate format using the CD73 Inhibitor Screening Assay Kit in accordance with the manufacturer's instructions (cat no. 72055, BPS Bioscience). Briefly, test inhibitors and the positive control (AB680; Lawson et al., 2020) were incubated in assay buffer (0.1 ng/μL, 20 μL/well) in the presence of AMP (10 μL/well, final 100 μM) and recombinant human CD73 enzyme (0.1 ng/μL) for 25 min at 37° C. Colorimetric detection reagent was then added (100 μL/well) and the free phosphate from the CD73 reaction was measured by reading the absorbance at 630 nm using the EnSpire multimode plate reader (PerkinElmer). Wells without test inhibitor (CD73 positive control) were included on each plate. Blank wells without test inhibitor and CD73 were subtracted from all wells. Percent CD73 inhibition versus inhibitor concentration was plotted using GraphPad Prism Version 8 and IC₅₀'s calculated using nonlinear regression analysis (Sigmoidal, 4 PL). Conjugate 133a (Int-259 conjugated to the Fc of SEQ ID NO: 13) and Conjugate 133b (Int-259 conjugated to the Fc of SEQ ID NO: 80) had single digit nM potency in the cell-free CD73 inhibition assay with IC₅₀'s of 8.8 and 10.4 nM, respectively (FIG. 1A). Further, both conjugates had superior potency to the Int alone (Int-258), which had an IC₅₀ of 26.1 nM. In addition, Conjugate 172a (Int-305 conjugated to the Fc of SEQ ID NO: 80) had single digit nM potency in the cell-free CD73 inhibition assay with an IC₅₀ of 9.1 nM (FIG. 1B), which was within approximately 2-7-fold of small molecule benchmark inhibitors including AB680 (IC₅₀ 1.9 nM), SHR170008 (IC₅₀ 4.1 nM) and OP-5244 (IC₅₀ 1.3 nM) (FIG. 1B). Notably, mupadolimab did not inhibit soluble, cell-free CD73 as shown previously (Miller et al., 2022 PMID: 36600561) (FIG. 1C). Oleclumab demonstrates incomplete CD73 inhibition reaching maximal 80% and a hook effect at higher concentrations resulting in decreased CD73 inhibition. Therefore, an IC50 could not be calculated for Oleclumab (FIG. 1C).

### Cell-Based CD73 Enzyme Inhibition Assay: Inhibition of Surface-Expressed CD73 on MDA-MB231 Cells (Conjugates 133a and 133b)

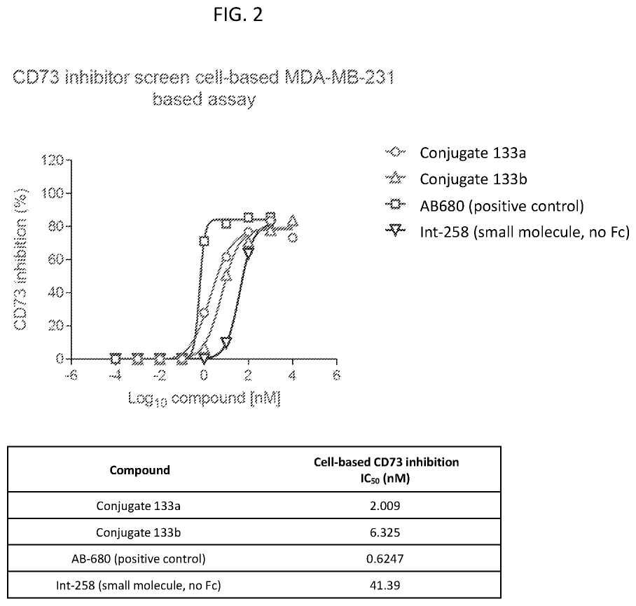

The cell-based CD73 enzyme inhibition assay was performed essentially as described above for the cell-free assay, with the following modifications. MDA-MB-231 human breast adenocarcinoma cells (ATCC CRM-HTB-26) were seeded at 5×10³ cells/well into 96-well tissue culture-treated plates and incubated overnight at 37° C., 5% CO₂. Cells were incubated with test inhibitors and AMP (100 μM) for 3 h at 37° C., 5% CO₂ to allow for interaction with CD73 expressed on the MDA-MB-231 cell surface. The CD73 activity in the supernatant was quantified using the CellTiter-Glo kit (cat no. G7571, Promega) as previously described (Sachsenmeier et al., Development of a novel ectonucleotidase assay suitable for high-throughput screening. J Biomol Screen. 2012 August; 17(7):993-8). Percent CD73 inhibition versus concentration were plotted using GraphPad Prism Version 8 and IC₅₀'s calculated using nonlinear regression analysis (Sigmoidal, 4 PL). Conjugate 133a (Int-259 conjugated to the Fc of SEQ ID NO: 13) and Conjugate 133b (Int-259 conjugated to the Fc of SEQ ID NO: 80) had single digit nM potency in the cell-based CD73 inhibition assay with IC₅₀'s of 2.0 and 6.3 nM, respectively (FIG. 2). Further, both conjugates had superior potency to the Int alone (Int-258), which had an IC₅₀ of 41.4 nM.

### Cell-Based CD73 Enzyme Inhibition Assay: Inhibition of Surface-Expressed CD73 on MDA-MB231 Cells (Conjugate 172a)

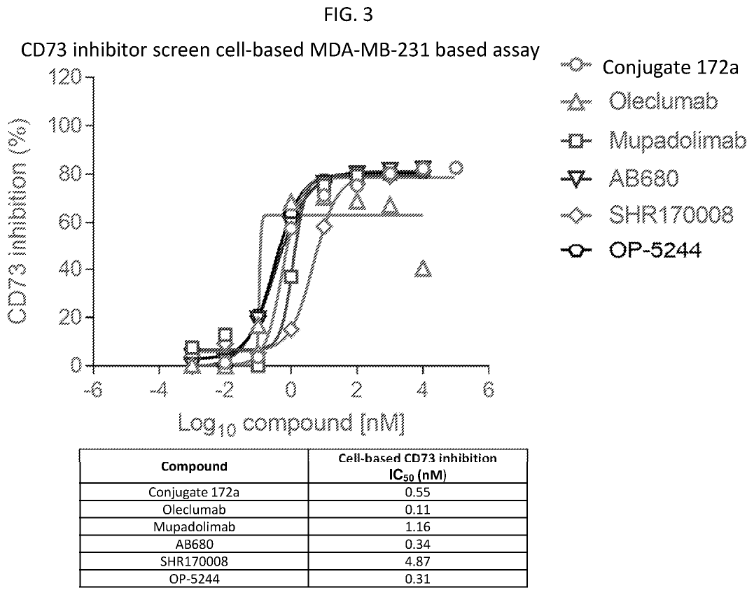

The cell-based CD73 enzyme inhibition assay was performed essentially as described above for the cell-free assay, with the following modifications. MDA-MB-231 human breast adenocarcinoma cells (ATCC CRM-HTB-26) were seeded at 2×10⁴ cells/well/100 μL into 96-well tissue culture-treated plates (Corning Costar cat no. 3596) and incubated overnight at 37° C., 5% CO₂. Cells were incubated with test inhibitors (50 μL/well) and AMP (25 μL/well, final 400 μM) for 3 h at 37° C., 5% CO₂ to allow for interaction with CD73 expressed on the MDA-MB-231 cell surface. The 96-well plate was then centrifuged at 125×g for 5 min at room temperature and ATP (25 μL/well, 100 μM final) was combined with supernatant (25 μL/well) in a white opaque 96-well plate (Corning Costar cat no. 3912). The CD73 activity in the supernatant was quantified using the CellTiter-Glo kit (Promega cat no. G7571) as previously described (Sachsenmeier et al., Development of a novel ectonucleotidase assay suitable for high-throughput screening. J Biomol Screen. 2012 August; 17(7):993-8). Percent CD73 inhibition versus inhibitor concentration were plotted using GraphPad Prism Version 8 and IC₅₀'s calculated using nonlinear regression analysis (Sigmoidal, 4 PL). Conjugate 172a demonstrated sub-nanomolar potency in the cell-based CD73 inhibition assay with an IC₅₀ of 0.55 nM (FIG. 3) with comparable potency to the anti-CD73 monoclonal antibody controls Oleclumab (IC₅₀ 0.11 nM) or Mupadolimab (IC₅₀ 1.16 nM), as well as small molecules AB680 (IC₅₀ 0.34 nM) and OP-5244 (IC₅₀ 0.31 nM). Conjugate 172a demonstrated improved potency compared to the small molecule SHR170008 (IC₅₀ 4.87 nM) (FIG. 3). The ability of Conjugate 172a to inhibit soluble and membrane anchored differentiates itself from comparator monoclonal antibodies, which only weakly inhibit soluble CD73.

### Human Immune Cell-Based CD73 Enzyme Inhibition Assay: Inhibition of Surface-Expressed CD73 on Human PBMCs and T Cells

The human immune cell-based CD73 enzyme inhibition assay was performed essentially as described above For the cell-free assay, with the following modifications. Human PBMCs (cat. no. HUMAN-PBMC-U-211140) or T cells (cat. no. 72400, Stem Cell Tech) were seeded at 8×10³ cells/well into 96-well tissue culture-treated plates. Test articles were added at concentrations ranging from 0.00001 to 1,000 nM followed by 15 min incubation of cells and text articles at 37° C., 5% CO₂. Next, cells were incubated with test articles and AMP (300 μM) for 2.5-3 h at 37° C., 5% CO₂ to allow for interaction with CD73. The CD73 activity in the supernatant was quantified using PiColorLock™ Phosphate detection reagent (cat no. 303-0125, Novus Bio) as previously described (Lawson et al., Discovery of AB680: A Potent and Selective Inhibitor of CD73. J Med Chem. 2020 Oct. 22; 63(20):11448-11468). Percent CD73 inhibition versus concentration were plotted using GraphPad Prism Version 8 and IC₅₀'s calculated using nonlinear regression analysis ([inhibitor]vs normalized response). Conjugate 133a and Conjugate 133b had pM potency in the immune cell based CD73 inhibition assay, the results of which are provided in Table 3.

TABLE 3
Inhibition of CD73 on human PBMCs and T cells

| | | CD73 inhibition |
|---|---|---|
| Experiment | Compounds | IC₅₀ (nM) |
| PBMCs | Conjugate 133a | 0.035 |
| | AB-680 | <0.000001 |
| | OP-5244 | 0.02 |
| | Int-259 (small molec only) | 2.87 |
| | SEQ ID NO: 80 (Fc only) | >1,000 |
| T Cells - | Conjugate 133a | 0.076 |
| Experiment 1 | AB-680 | <0.000001 |
| | Int-259 (small molec only) | 1.47 |
| T Cells - | Conjugate 133b | 0.0036 |
| Experiment 2 | Int-259 (small molec only) | 0.12 |

Conjugate 133a had an IC₅₀ of 0.035 nM (PBMCs) and 0.076 nM (T cells). Conjugate 133b had an IC₅₀ of 0.0036 nM (T cells). Further, both conjugates had superior potency to the small molecule alone (Int-259), which had an IC₅₀ of 1.47 nM or 0.12 nM (T cells) or 2.87 nM (PBMCs). Small molecule comparator drugs AB680 and OP5244 (Du et al., Orally Bioavailable Small-Molecule CD73 Inhibitor (OP-5244) Reverses Immunosuppression through Blockade of Adenosine Production. J Med Chem. 2020 Sep. 24; 63(18):10433-10459), currently in clinical testing, have an IC50 of <0.00001 nM (T cells or PBMCs) or 0.02 nM (PBMCs), respectively.

### PBMC Activation Assay by Flow Cytometry Using Conjugate 172a

Human PBMCs (StemCell Technologies cat no. 70025) prepared in ImmunoCult™—XF T cell Expansion medium (StemCell Technologies cat no. 10981) were seeded at 1×10⁵ cells per well (100 μL of 1×10⁶ cells/mL) in the presence or absence of T cell activator anti-CD3/CD28/CD2 (StemCell Technologies cat no. 10970) in 96 well round bottom plates (Corning cat no. 3788). Ten-fold serial dilutions of each test article was prepared, resulting in final concentrations ranging from 0.1 nM-10,000 nM. 50 μL of test article was added to the cells, followed by 50 μL of adenosine 5′ monophosphate (AMP; MilliporeSigma cat no. A1752) for a final concentration of 30 μM. Plates were incubated at 37° C. with 5% CO₂ for 72 h. After 72 h the cells were centrifuged for 5 min at 300×g at 4° C. then washed twice with cell staining buffer (BioLegend cat no. 420201). Cells were incubated with Fc block (Unlabeled, Clone: 3070; BD Biosciences cat no. 5642220) for 10 min at 4° C. Cells were stained with anti-human CD25 antibody, clone 2A3, PE (StemCell Technologies cat no. 60153PE) for 20 min at 4° C. protected from light. Cells were washed twice with cell staining buffer and resuspended in 200 μL of cell staining buffer. Samples were acquired using a Cytek Northern Lights flow cytometry system. CD25⁺ by flow cytometry was used as the readout to measure PBMC activation after AMP inhibition. Samples were analyzed using FlowJo version 10. Percent rescue (x-axis) versus inhibitor concentration (y-axis) was plotted using GraphPad Prism Version 6 and EC₅₀ values were generated using nonlinear regression analysis for total PBMCs or CD8⁺ T cells using CD25⁺ as read-out or granzyme B⁺.

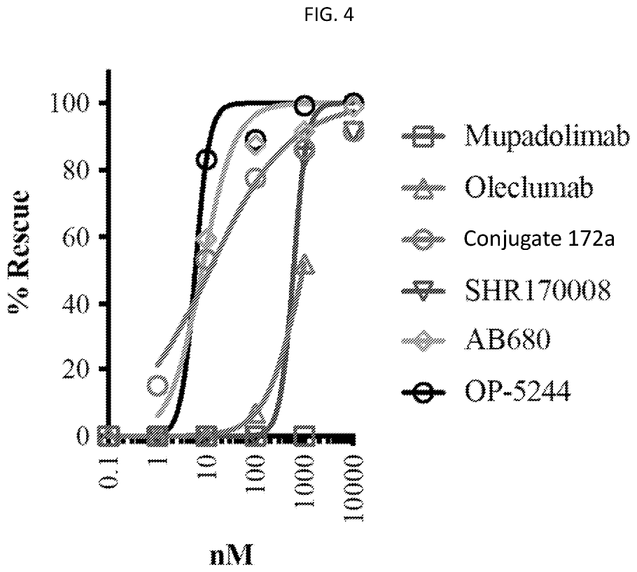

Conjugate 172a and five additional control compounds were tested to determine their ability to re-activate PBMCs that were inhibited by 30 μM AMP. Conjugate 172a showed potent activity against 30 μM AMP, with an EC₅₀ value of 6 nM. This was comparable to two of the most potent small molecule control compounds, AB680 and OP5244, which generated EC₅₀ values of 9 nM and 8 nM, respectively. Conjugate 172a was significantly more potent than the three remaining control compounds, including small molecule CD73 inhibitor SHR170008, and the anti-CD73 monoclonal antibodies Mupadolimab and Oleclumab (Tables 4 and 5 and FIG. 4).

TABLE 4
Activity of test articles targeting CD73 in a
PBMC activation assay using CD25+ as a read-out

| Test article | 30 μM AMP EC₅₀ [nM] |
|---|---|
| Conjugate 172a | 6 |
| SHR170008 | 374 |
| AB680 | 9 |
| OP-5244 | 8 |
| Oleclumab | >1,000 |
| Mupadolimab | 576 |

TABLE 5
Activity of test articles targeting CD73 in a PBMC activation assay using
CD8⁺CD25⁺ and CD8⁺granzyme B⁺ as a read-out.

| Test article | CD8⁺CD25⁺ EC₅₀ [nM] | CD8⁺granzyme B⁺ EC₅₀ [nM] |
|---|---|---|
| Conjugate 172a | 11.6 | 1.2 |
| SHR170008 | 597.7 | 212.2 |
| AB680 | 8.1 | 1.4 |
| OP-5244 | 5.9 | 1.4 |
| Oleclumab | >1,000 | >1,000 |
| Mupadolimab | 932.3 | 63.1 |

CD73 Enzyme Inhibition Assay with Human PBMCs and Mouse 4T1 Cells

The cell-based CD73 enzyme inhibition assay was performed in a 96-well plate format using the PiColorLock phosphate assay kit in accordance with the manufacturer's instructions (Abcam cat no. 270004). 4T1 mouse cells are a model for stage IV human breast cancer (ATCC CRL-2539) and human peripheral blood mononuclear cells (PBMCs; StemCell Technologies cat no. 70025) from three different donors were assayed. 4T1 cells were washed three times with a phosphate-free CD73 buffer (Lawson et al., 2020), detached from tissue-culture flasks using a cell scraper, and seeded at 5×10³ cells/well into 96-well tissue culture-treated plates. Human PBMCs were washed three times with phosphate-free CD73 buffer at 125×g for 10 min at room temperature and seeded at 1×10⁵ cells/well into 96-well tissue culture-treated plates. Test articles were tested from 0.001 nM to 1,000 nM (4T1 cells) or 0.0001 nM to 100 nM (PBMCs) and incubated with cells for 30 min at 37° C., 5% CO₂. AMP (100 μM final concentration) was added to cells and test articles, and the reaction incubated for 3 h and 24 h at 37° C., 5% CO₂. The conversion of AMP to adenosine and phosphate in cell-free supernatant was quantified using the PiColorLock kit. Colorimetric detection reagent was added and the phosphate production was measured by reading the absorbance at 635 nm with the EnSpire multimode plate reader (PerkinElmer). Cells without test article (positive control) were included on each plate. Control wells without test article and cells (background) were subtracted from all wells. Percent CD73 inhibition versus inhibitor concentration was plotted using GraphPad Prism Version 8 and IC₅₀'s calculated using nonlinear regression analysis (Sigmoidal, 4 PL).

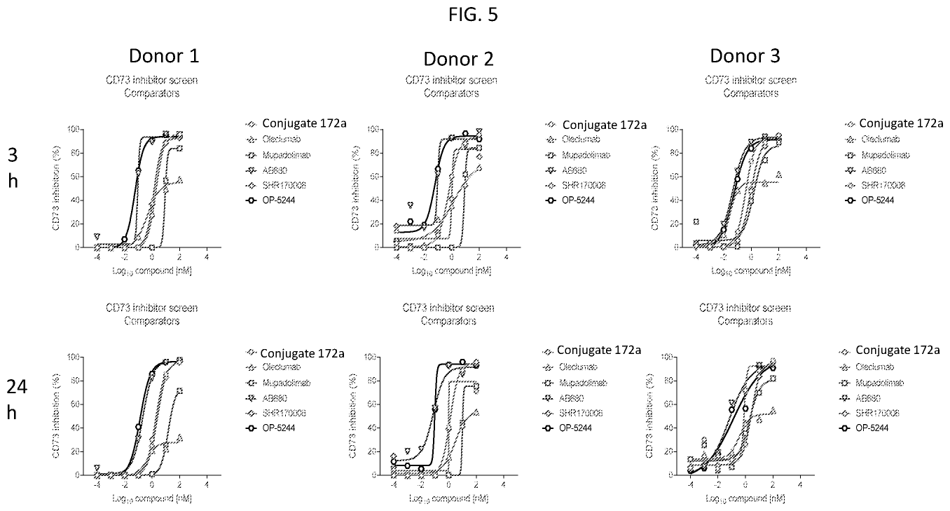

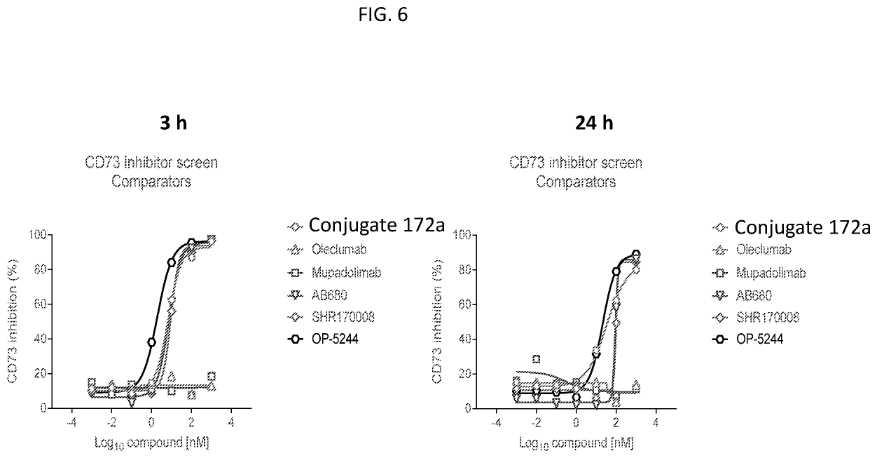

Conjugate 172a demonstrated potent and complete CD73 inhibition similar to small molecule inhibitors, AB680, P5244 and SHR70008 in human PBMC donors. Conjugate 172a demonstrated increased potency as compared to anti-CD73 monoclonal antibodies, Oleclumab or Mupadolimab in human PBMC donors. Oleclumab only partially inhibited CD73 in human PBMC donors (FIG. 5). Compared to other test articles, Conjugate 172a exhibited a negligible increase in median IC₅₀ from 3 h time point to the 24 h with median C₅₀'s of 0.8 nM to 1.17 nM in three unique human PBMC donors (Table 6). Conjugate 172a demonstrated potent and complete CD73 inhibition similar to small molecule inhibitors, AB680, P5244 and SHR170008 in 4T1 cancer cells. The two anti-CD73 monoclonal antibodies, Oleclumab or Mupadolimab, had a IC₅₀>100 nM in 4T1 cancer cells (FIG. 6). Compared to other test articles, Conjugate 172a exhibited a modest increase in IC₅₀ from 3 h time point to the 24 h with IC₅₀'s of 6.21 nM to 38.69 nM in 4T1 cancer cells (Table 7).

TABLE 6
Median IC₅₀ [nM] for CD73 inhibition of Conjugate
172a and comparators from three unique PBMC donors.

| | 3 h median | 24 h median | |
|---|---|---|---|
| Test article | IC₅₀ [nM] (range) | IC₅₀ [nM] (range) | |
| Conjugate 172a | 0.8 (0.46-2.07) | 1.17 | (0.47-2.15) |
| AB680 | 0.05 (0.04-0.09) | 0.1 | (0.09-0.23) |
| OP-5244 | 0.07 (0.04-0.08) | 0.15 | (0.1-0.18) |
| SHR170008 | 1.45 (1.14-1.49) | 2.36 | (2.34-2.67) |
| Oleclumab | 7.26 (3.12-16.6) | 89.41 | (20.14->100) |
| Mupadolimab | 9.27 (2.4-9.78) | 29.85 | (3.04-35.71) |

TABLE 7
IC₅₀ [nM] for CD73 inhibition of Conjugate
172a and comparators in 4T1 cancer cells.

| Test article | 3 h IC₅₀ [nM] | 24 h IC₅₀ [nM] |
|---|---|---|
| Conjugate 172a | 6.21 | 38.69 |
| AB680 | 6.79 | 85.7 |
| OP-5244 | 1.67 | 24.02 |
| SHR170008 | 8.20 | 114 |
| Oleclumab | >100 | >100 |
| Mupadolimab | >100 | >100 |

### Binding of Conjugate 172a to Human PBMCs

Naïve human PBMCs (StemCell Technologies cat no. 70025) prepared in ImmunoCult™—XF T cell Expansion medium (StemCell Technologies cat no. 10981) were seeded at 1×10⁵ cells per well in 96 well round bottom plates. Test articles were labeled with AlexaFluor™ 488 (AF488) Antibody labeling kit (Thermo Fisher Scientific cat no. A20181) following manufacturer's instructions. Ten-fold serial dilutions of labeled test articles were prepared, resulting in final concentrations ranging from 0.001 nM-100 nM. Test articles were incubated with PBMCs for 1 h at 37° C., 5% CO₂. After 1 h, the cells were centrifuged for 3 min at 300×g at room temperature then washed twice with cell staining buffer (BioLegend cat no. 420201). Cells were incubated with Fc block (Unlabeled, Clone: 3070; BD Biosciences cat no. 5642220) for 10 min. Cells were stained with anti-human CD3 (clone: UCHT1) PerCP/Cy5.5 (BioLegend cat no. 300430), anti-human CD4 (clone: SK3) PE-Fire810 (BioLegend cat no. 344677), anti-human CD8 (clone: SK1) PerCP (BioLegend cat no. 344708) and, anti-human CD19 (clone: HIB19) PE (StemCell Technologies cat no. 60005PE) for 15-20 min at 4° C. protected from light. Cells were washed twice with cell staining buffer and resuspended. Samples were acquired using a Cytek Northern Lights flow cytometer. Binding to CD73 was determined by AF488⁺ signal gated on B cells (CD3⁻CD19⁺) and T cells (CD3⁺CD8⁺). Samples were analyzed using FlowJo version 10 and IC₅₀ values were generated using non-linear regression (4 PL) analysis in GraphPad Prism 9.

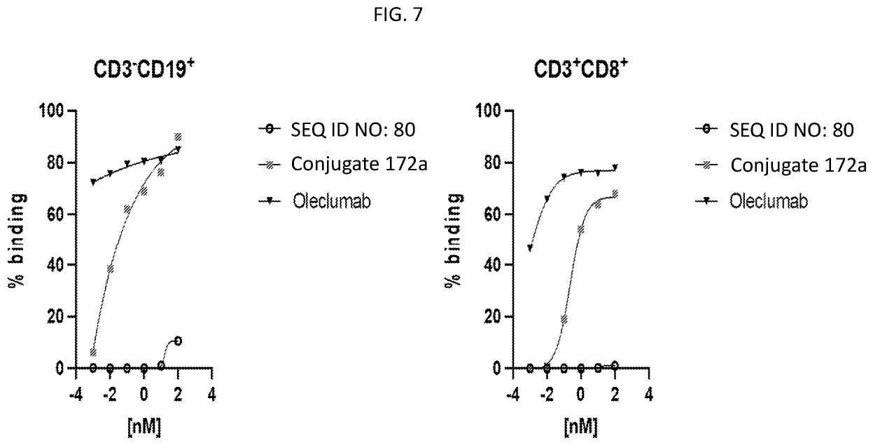

Conjugate 172a demonstrated potent, quantitative binding to B cells (CD3⁻CD19⁺) and CD8⁺ T cells (CD3⁺CD8⁺) expressing CD73 (FIG. 7) with an IC₅₀ of 0.075 nM and 2.7 nM, respectively (Table 8). SEQ ID NO: 80 (Conjugate 172a lacking the Int) was used as a negative control, thus demonstrating that Conjugate 172a exhibits targeted binding to B cells.

TABLE 8
IC₅₀ [nM] for binding of Conjugate 172a and
comparators to PBMCs derived from gMFI (AF488).

| Test article | CD3⁻CD19⁺ IC₅₀ [nM] | CD3⁺CD8⁺ IC₅₀ [nM] |
|---|---|---|
| SEQ ID NO: 80 | >100 | >100 |
| Conjugate 172a | 0.075 | 2.7 |
| Oleclumab | <0.001 | <0.001 |

### B Cell Activation of Conjugate 172a to Human PBMCs

Naïve, human PBMCs (StemCell Technologies cat no. 70025) prepared in ImmunoCult™ Human B Cell Expansion medium (StemCell Technologies cat no. 100-0645) were seeded at 1×10⁵ cells per well in 96-well round bottom plates. Ten-fold serial dilutions of labeled test articles were prepared, resulting in final concentrations ranging from 0.001 nM-1,000 nM. Test articles were incubated with PBMCs for 24 h at 37° C., 5% CO₂. After 24 h, the cells were centrifuged for 3 min at 300×g at room temperature then washed twice with cell staining buffer (BioLegend cat no. 420201). Cells were incubated with Fc block (Unlabeled, Clone: 3070; BD Biosciences cat no. 5642220) for 10 min. Cells were stained with anti-human CD3 (clone: UCHT1) PerCP/Cy5.5 (BioLegend cat no. 300430), anti-human CD19 (clone: HIB19) AlexaFluor™ 488 (StemCell Technologies cat no. 60005AD) and anti-human CD69 (clone FN50) (BioLegend cat no. 310928) for 15-20 min at 4° C. protected from light. Cells were washed twice with cell staining buffer and resuspended. Samples were acquired using a Cytek Northern Lights flow cytometer. B cell activation was determined by gMFI of CD69 signal gated on B cells (CD3⁻CD19⁺). Samples were analyzed using FlowJo version 10 and graphs generated in GraphPad Prism 9.

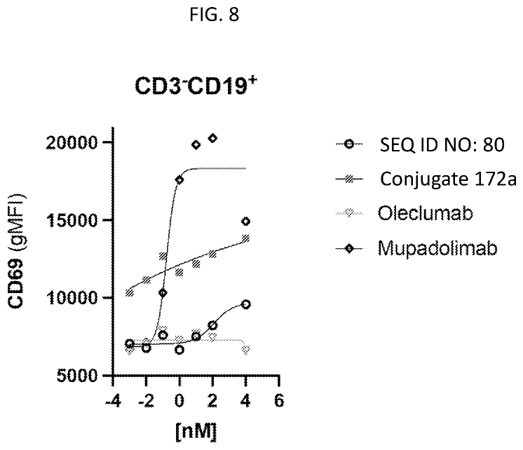

Conjugate 172a showed modest activation of B cells (CD3⁻CD19⁺) quantified by CD69 expression (FIG. 8). Mupadolimab, an anti-CD73 monoclonal antibody with B cell activating properties (Miller et al., Anti-CD73 antibody activates human B cells, enhances humoral responses and induces redistribution of B cells in patients with cancer. Journal for ImmunoTherapy of Cancer. 2022), also showed B cell activation quantified by CD69 expression. The Fc only control, SEQ ID NO: 80, and another anti-CD73 monoclonal antibody that binds a different CD73 epitope than Conjugate 172a, Oleclumab, did not induce CD69 expression/B cell activation.

### AMP Competition and Catalytic Site Inhibition Assay by Flow Cytometry

The binding of drug-Fc conjugates (DFCs) to the MDA-MB-231 (ATCC CRM-HTB-26) cancer cell line was determined in the presence of substrates and a competitive inhibitor using the following protocol. Cancer cells were washed 2× with PBS (Gibco cat. no 10010023), detached from tissue culture flask via cell scraper (Fisher cat no. 08-771-1B) and strained using a 70 μm cell strainer (StemCell Technologies cat. no 27260). Cells were seeded in 100 μL cell staining buffer (BioLegend cat no. 420801) at 0.2-1×10⁵ cells per well in a nontreated 96 well round bottom plate (Corning cat. no 3788). Serial dilutions of DFCs from 0.000004 nM-400 nM (4× final concentration) at 50 μL/well in the presence or absence of small molecule at 4, 40, 400 nM or adenosine monophosphate (AMP; MilliporeSigma cat. no A9396) at 40, 400 or 4,000 μM, respectively. Cells and test articles were incubated with for 37° C. with 5% CO₂ for 60 min. Cells were then centrifuged at 300×g for 3 min at room temperature and the supernatant was discarded. Cells were washed twice at 300×g for 3 min. Cells were stained with a polyclonal anti-human Fc antibody (Invitrogen cat. no 12-4998-82) at 1.25 μL per 100 μL/well. Cells were resuspended and incubated for 15-20 min at 4° C. protected from light. Following the incubation, 100 μL/well of cell staining buffer was added and the plate was centrifuged at 300×g for 3 min. The supernatant was discarded, and an additional 200 μL/well of cell staining buffer was added to plate. Cells were resuspended, then centrifuged at 300×g for 5 min. Cells were washed three times at 300×g for 3 min. Samples were acquired on Northern Lights (Cytek) flow cytometer. The gMFI anti-human Fc labeled cells was determined in FlowJo (v.10) and graphed in Prism. The IC₅₀'s for each test article was calculated using the 4 PL non-linear regression analysis in GraphPad Prism software.

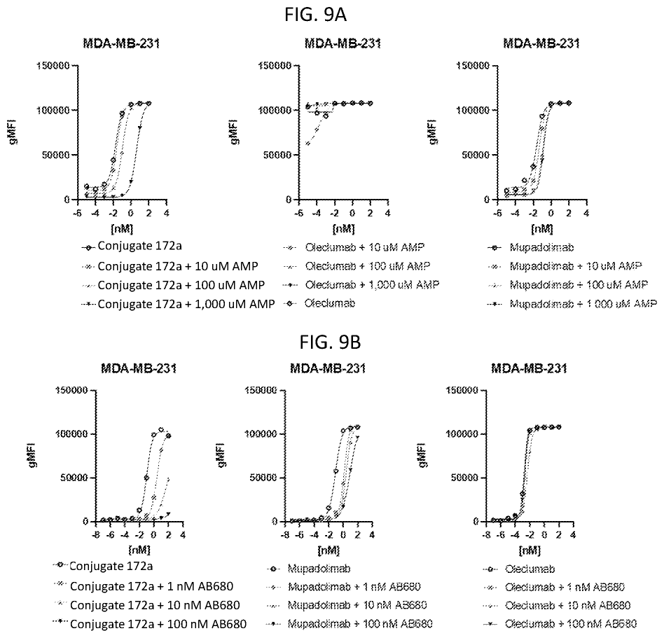

Conjugate 172a binding to MDA-MB-231 cells (IC₅₀ 0.018 nM) was comparable to Mupadolimab (IC₅₀ 0.025 nM) (FIG. 9A, Table 9), while Oleclumab (IC₅₀ 0.0068 nM) binding to MDA-MB-231 was more potent. The binding of Conjugate 172a in the presence of AMP was reduced in an AMP-dependent manner demonstrating that Conjugate 172a is an AMP-competitive CD73 inhibitor. Similarly, the binding of Mupadolimab was reduced in the presence to AMP to CD73 as has been reported in a competition study with a non-hydrolyzable AMP analogue (Miller et al., Anti-CD73 antibody activates human B cells, enhances humoral responses and induces redistribution of B cells in patients with cancer. Journal for ImmunoTherapy of Cancer. 2022). No difference in binding was observed for Oleclumab (an allosteric inhibitor) in the presence of AMP.

Conjugate 172a binding to MDA-MB-231 cells (IC₅₀ 0.11 nM) was comparable to Mupadolimab (IC₅₀ 0.09 nM) (FIG. 9B, Table 10), but less potent than Oleclumab (IC₅₀ 0.017 nM). The binding of Conjugate 172a in the presence of increasing concentrations of a small molecule CD73 inhibitor, AB680, was reduced in a dose-dependent manner, demonstrating that Conjugate 172a is a catalytic site CD73 inhibitor. Similarly, the binding of Mupadolimab was reduced in the presence to AB680 to CD73. No difference in binding has been observed for Oleclumab (an allosteric inhibitor) in the presence of AB680. These studies highlight the potent inhibitory activity of Conjugate 172a versus CD73 and demonstrate that Conjugate 172a is an AMP-competitive inhibitor of the enzyme.

TABLE 9
IC₅₀ of binding of Conjugate 172a, Oleclumab and Mupadolimab in
the presence and absence of AMP to human MDA-MB-231 cancer cells.

| | | No AMP | 10 μM AMP | 100 μM AMP | 1,000 μM AMP |
|---|---|---|---|---|---|
| Test article | DAR | IC₅₀ [nM] | IC₅₀ [nM] | IC₅₀ [nM] | IC₅₀ [nM] |
| Conjugate 172a | 7.7 | 0.018 | 0.026 | 0.11 | 4.31 |
| Oleclumab | n/a | 0.0068 | 0.00017 | 0.00095 | <0.00001 |
| Mupadolimab | n/a | 0.025 | 0.048 | 0.12 | 0.14 |

TABLE 10
IC₅₀ of binding of Conjugate 172a, Oleclumab and Mupadolimab
in the presence and absence of small molecule CD73 inhibitor,
AB680, to human MDA-MB-231 cancer cells.

| | | No AMP | 1 nM AB680 | 10 nM AB680 | 100 nM AB680 |
|---|---|---|---|---|---|
| Test article | DAR | IC₅₀ [nM] | IC₅₀ [nM] | IC₅₀ [nM] | IC₅₀ [nM] |
| Conjugate 172a | 7.7 | 0.11 | 2.70 | 42.42 | >100.00 |
| Oleclumab | n/a | 0.0017 | 0.0025 | 0.0048 | 0.0020 |
| Mupadolimab | n/a | 0.09 | 1.39 | 2.59 | 8.48 |

### Rescue of T Cell Activation in the Presence of AMP

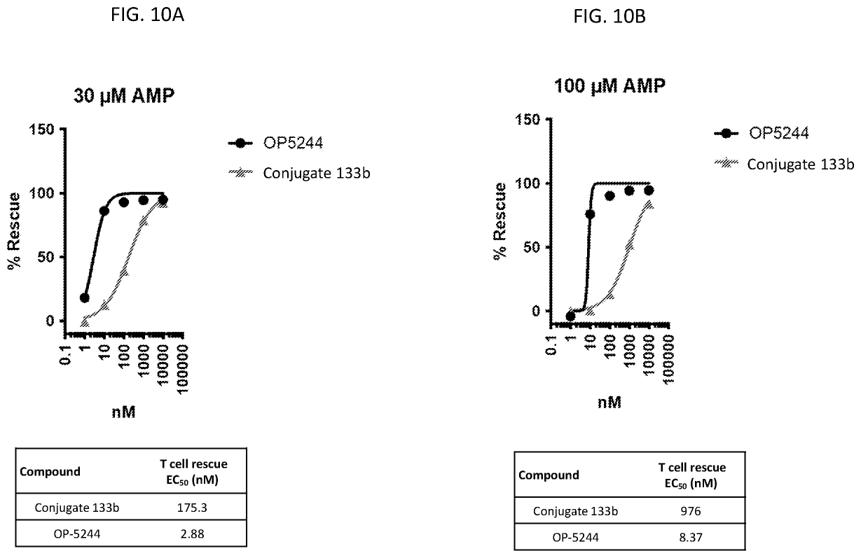

Human T cells (cat. no. 72400, Stem Cell Tech) were activated with anti-CD3/CD28/CD2 T cell activator (cat. no. 10970, Stem Cell Technologies) and seeded at 1×10⁵ cells/well in ImmunoCult™—XF T Cell Expansion medium into 96-well tissue culture-treated plates. Test articles were added at concentrations ranging from 1 to 10,000 nM. Next, adenosine 5′-monophosphate (AMP; cat. no 01930, Sigma) was added at 30 μM or 100 μM. Cells, test articles and AMP were incubated at 37° C., 5% CO₂ for 72 h. After 72 h, cell supernatant was removed, and T cells were washed in staining buffer prior to staining for CD25 with anti-human CD25 PE antibody (cat. no 60153PE, Stem Cell Technologies, clone 2A3). After staining, cells were washed in staining buffer. 10,000 events were collected on flow cytometer Northern Lights (Cytek). Percent rescue versus concentration were plotted using GraphPad Prism Version 8 and EC₅₀'s calculated using nonlinear regression analysis ([inhibitor]vs normalized response). Conjugate 133b in the presence of 30 μM AMP or 100 μM AMP had EC₅₀ of 175.3 nM or 976 nM, respectively (FIG. 10A and FIG. 10B). Small molecule comparator drugs OP5244 (Du et al., 2020), currently in clinical testing, had an EC₅₀ of 2.88 nM or 8.37 nM, respectively.

CD73 expressed on T-cells converts AMP to adenosine, which results in T-cell anergy. This study demonstrates that conjugate 133b reactivates T-cells suppressed by adenosine via inhibition of CD73.

### CD73 Internalization Assay

The ability of Conjugate 172a to induce receptor-mediated internalization of CD73 into the human breast adenocarcinoma cell line, MDA-MB-231 (ATCC CRM-HTB-26), was measured using the Fab-ZAP saporin conjugate assay kit (Advanced Targeting Systems cat no. IT-65-25) as previously described (Hay et al., 2016). Cells were plated into white tissue culture-treated 96 well plates (Corning cat no. 3917) at 5×10³ to 1×10⁴ cells/well in 100 μL in RPMI (Gibco cat no. 11875093)+10% FBS (Gibco cat no. 16140071) and incubated overnight at 37° C. with 5% CO₂. Serial dilutions of test article (TA) from 0.00002 nM-200 nM (2× final concentration) were pre-incubated with 100 nM Fab-ZAP reagent (2× final concentration) for 30 min at room temperature. Cells were washed once with 150 μL/well 1×PBS (Gibco cat no. 10010023), followed by the addition of 50 μL/well of media and 50 μL/well TA/Fab-ZAP mix. Cells were then incubated for 72 h at 37° C. with 5% CO₂ and viability was determined after 72 h via the CellTiter-Glo assay (Promega cat no. G7570) following the manufacturer's instructions. Cell viability RLU versus log₁₀ nM drug concentration was plotted using GraphPad Prism. The % CD73 internalization was calculated using the following formula: % CD73 internalization=[1−RLU (sample)/RLU (untreated cells)]×100%. The EC₅₀'s for each TA was calculated using the 4 PL non-linear regression analysis in GraphPad Prism software.

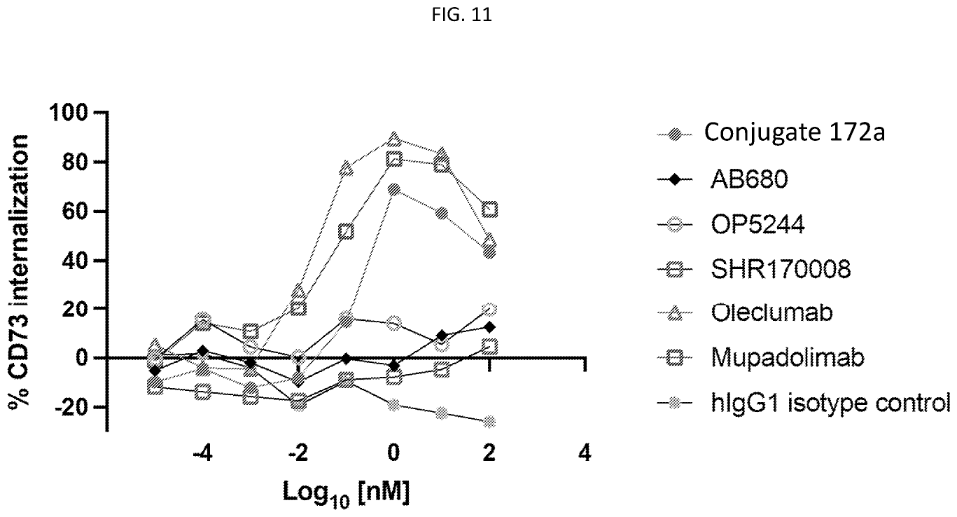

Conjugate 172a and the anti-CD73 monoclonal antibodies demonstrated potent sub-nanomolar CD73 internalization activity (FIG. 11) resulting in >60% receptor internalization on the MDA-MB-231 cell line. The EC₅₀ for Oleclumab (EC₅₀ 0.02 nM) and Mupadolimab (EC₅₀ 0.06 nM) was approximately 5- and 2-fold lower as compared to Conjugate 172a (EC₅₀ 0.11 nM). The human IgG1 isotype control did not exhibit any internalization activity up to the maximum concentration tested (Table 11). As expected, small molecule inhibitors tested (AB680, OP-5244 and SHR170008) did not induce receptor internalization (FIG. 11). The receptor internalization activity exhibited by Conjugate 172a may enhance anti-tumor activity versus tumor cells that express high levels of CD73.

TABLE 11
CD73 internalization EC₅₀s of Conjugate 172a, Oleclumab,
Mupadolimab, hIgG1 Fc control and full-length hIgG1
isotype control into MDA-MB-231 cancer cells.

| Test article | DAR | Cell internalization EC₅₀ (nM) |
|---|---|---|
| Conjugate 172a | 7.7 | 0.11 |
| Oleclumab | n/a | 0.02 |
| Mupadolimab | n/a | 0.06 |
| hIgG1 isotype control | n/a | >100 |

### CD73 Binding Assay by Flow Cytometry

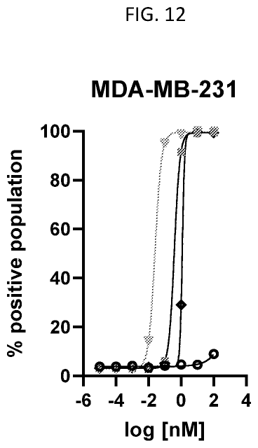

The binding of Conjugate 172a to the MDA-MB-231 (ATCC CRM-HTB-26 breast cancer cell line), which expresses high levels of CD73, was determined using the following protocol. Cancer cells were washed 2× with PBS (Gibco cat. no 10010023), detached from tissue culture flask via cell scraper (Fisher cat no. 08-771-1B) and strained using a 70 μm cell strainer (StemCell Technologies cat. no 27260). Cells were seeded in 100 μL cell staining buffer (BioLegend cat no. 420801) at 5×10⁴ cells per well in a nontreated 96 well round bottom plate (Corning cat. no 3788). Serial dilutions of DFCs from 0.00002 nM-200 nM (2× final concentration) at 100 μL/well were incubated with plated cells for 37° C. with 5% CO₂ for 30 min. Cells were then centrifuged at 300×g for 5 min at room temperature and the supernatant was discarded. 200 μL/well of cell staining buffer was added to plate and cells were resuspended, then centrifuged at 300×g for 5 min, and supernatant was discarded. Cells were stained with PE-anti-human Fc antibody (BioLegend cat. no 410708) at 1.25 μL per 100 μL/well. Cells were resuspended and incubated for 15-20 min at 4° C. protected from light. Following the incubation, 100 μL/well of cell staining buffer was added and the plate was centrifuged at 300×g for 5 min. The supernatant was discarded, and an additional 200 μL/well of cell staining buffer was added to plate. Cells were resuspended, then centrifuged at 300×g for 5 min. The supernatant was discarded, and cells were resuspended in 200 μL. Samples were acquired on Northern Lights (Cytek) flow cytometer. Percent anti-human Fc labeled cells was calculated in FlowJo (v.10) and graphed as % binding in Prism. The IC₅₀'s for each test article was calculated using the 4 PL non-linear regression analysis in GraphPad Prism software. Conjugate 172a binding to MDA-MB-231 cells (IC₅₀ 0.39 nM) was approximately 3-fold more potent than Mupdolimab (IC₅₀ 1.25 nM) (FIG. 12, Table 12). Oleclumab (IC₅₀ 0.03 nM) binding to MDA-MB-231 was approximately 13-fold more potent compared to Conjugate 172a. No binding of SEQ ID NO: 80 (the hlgG1 Fc carrier negative control) was detected as expected.

This data demonstrates that Conjugate 172a tightly binds to CD73 expressed on a human cancer cell line, with comparable binding affinity to CD73 binding monoclonal antibodies in clinical trials.

TABLE 12
Binding EC₅₀s of Conjugate 172a, SEQ ID NO:
80 (hIgG1 Fc control), Oleclumab and Mupadolimab
to human MDA-MB-231 cancer cells.

| Test article | DAR | EC₅₀ [nM] |
|---|---|---|
| Conjugate 172a | 7.7 | 0.39 |
| SEQ ID NO: 80 | n/a | >100 |
| Oleclumab | n/a | 0.03 |
| Mupadolimab | n/a | 1.25 |

7-Day Mouse Pharmacokinetic Study for Conjugate 133a at 10 mg/Kg Administered Intramuscularly

The mouse PK study was performed using male BALB/c mice 6 weeks of age (n=2 mice/group). Mice were given intramuscular (IM) injections of Conjugate 133a at 10 mg/kg (5 mL/kg dose volume). Animals were housed under standard IACUC approved housing conditions. At indicated times (0.25, 2, 4, 24, 48, 72, 144, and 168 h), animals were non-terminally bled (retro-orbital, cheek, or by tail vein) with blood collected in K₂EDTA tubes to prevent coagulation. Collected blood was centrifuged (2,000×g, for 10 min) and plasma withdrawn for analysis of Conjugate 133a concentrations overtime. The Fc plasma concentrations at each time point were measured by Fc-capture sandwich ELISA as follows. Nunc Maxisorp 96-well plates (cat no. 12-565-136, Fisher Scientific) were coated overnight at 4° C. with 0.1 μg/100 μL/well of goat anti-human IgG (Fcγ fragment specific; cat no. 109-005-098, Jackson ImmunoResearch) in carbonate coating buffer (cat no. C3041, MilliporeSigma). Plates were washed 3× with 300 μL/well PBS 0.05% Tween 20 (PBST) and blocked with 200 μL/well 5% non-fat dry milk (cat no. 9999S, Cell Signaling) in PBST for 1 h at room temperature with shaking (400 rpm). Three-fold serial dilutions of the plasma samples were plated at 100 μL/well and incubated at room temperature for 2 h with shaking (sample diluent: 2.5% non-fat dry milk in PBS 0.025% Tween 20+naïve mouse plasma final concentration of 1:100). Conjugate 133a standard curves ranging from 0.03 to 55 ng/mL in duplicate, were run on each plate. Following the 2 h incubation, plates were washed 3× with 300 μL/well PBST. Conjugate 133a bound to Fc on the plates was then probed with 100 μL/well of HRP conjugated anti-human IgG Fc F(ab′)2 (cat no. 709-036-098, Jackson ImmunoResearch) diluted 1:2,000 in sample diluent for 1 h at room temp with shaking (400 rpm). Plates were then washed 3× in 300 μL/well PBST and developed with 100 μL/well TMB substrate reagent (cat no. 555214, BD) for 7-8 minutes. The reaction was stopped with 100 μL/well 1 N H₂SO₄ and the absorbance read at 450 nm with an EnSpire multimode plate reader (PerkinElmer). Conjugate 133a in plasma samples was interpolated using GraphPad Prism Version 8 following nonlinear regression analysis (Sigmoidal, 4 PL analysis) of the standard curves. The resulting mean plasma concentrations of Conjugate 133a were then used to calculate the total AUC for each plasma concentration-time profile.

The CD73-capture ELISA was performed as follows to quantify the concentration of Fc with attached Int (intact drug). Nunc Maxisorp 96-well plates (cat no. 12-565-136, ThermoFisher) were coated with 0.2 μg/100 μL/well human CD73/NT5E protein, His-tag (HPLC-verified) (active enzyme) (cat no. CD3-H52H7, AcroBiosystems) in carbonate coating buffer (cat no. C3041, MilliporeSigma). Plates were washed 3× with 300 μL/well PBST and blocked with 200 μL/well 1% BSA (cat no. A5611, MilliporeSigma) in PBST for 1 h on an orbital plate shaker (400 rpm). Serial dilutions of the plasma samples were plated and incubated at room temperature for 2 h (sample diluent: 0.5% BSA in PBS 0.025% Tween 20+naïve BALB/c mouse plasma final concentration of 1:100). Conjugate 133a standard curves ranging from 0.006 to 500 ng/mL in duplicate were run on each plate. Following the 2 h incubation, plates were washed 3× in 300 μL/well PBST. Conjugate 133a bound to CD73 on the plates was then probed with 100 μL/well of HRP conjugated anti-human IgG Fc F(ab′)2 (709-036-098, Jackson) diluted 1:2,000 in sample diluent for 1 h at room temp. Plates were then washed 3× in 300 μL/well PBST and developed with 100 μL/well TMB substrate (cat no. 555214, BD) for 7-8 minutes. The reaction was stopped with 100 μL/well 1N H₂SO₄. Absorbance was read at 450 nm with an EnSpire multimode plate reader (PerkinElmer). Conjugate 133a in plasma samples was interpolated using GraphPad Prism Version 8 following nonlinear regression analysis (Sigmoidal, 4 PL analysis) of the standard curves. The resulting mean plasma concentrations of Conjugate 133a were then used to calculate the total AUC for each plasma concentration-time profile.

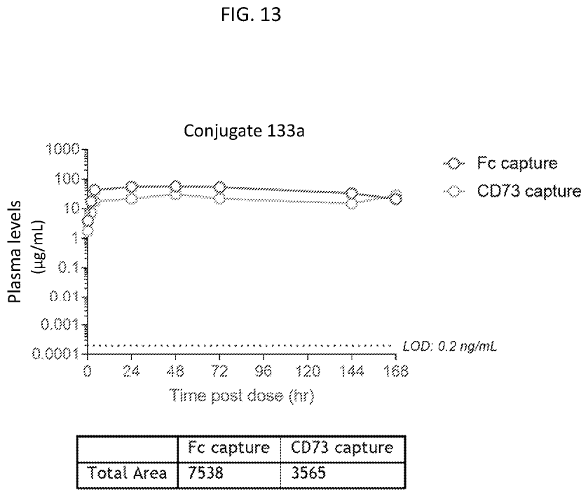

The 7-day mouse PK profile for Conjugate 133a dosed at 10 mg/kg IM is shown in FIG. 13. After 168 h, similar plasma exposure levels were observed for the CD73 (28.6 μg/mL) and Fc (21.5 μg/mL) capture assays. The AUCs for the CD73 (3565) and Fc (7538) captures were within approximately 2-fold of each other, suggesting minimal loss of the Int over time.

Seven-Day Mouse Pharmacokinetic Study for Conjugate 172a at 10 mg/Kg Administered Intramuscularly

A mouse PK study was performed using female BALB/c (CRL cat no. 028BALB/C) mice 6-9 weeks of age. Mice were injected intramuscularly (IM) with 10 mg/kg of test article (5 mL/kg dose volume). Animals were housed under standard IACUC approved housing conditions. At appropriate times animals were non-terminally bled (retro-orbital, cheek, or by tail vein) with blood collected in K₂EDTA tubes to prevent coagulation. Collected blood was centrifuged (2,000×g, for 10 min) and plasma withdrawn for analysis of test article concentrations overtime. Plasma concentrations at each time point were measured using two separate ELISA methods: a human CD73 protein capture with Fc-detection, and a Fc-capture method with Fc-detection to compare and confirm that the intact molecule (containing at least one targeting moiety (Int) remained stable in vivo. For the CD73-capture, Nunc Maxisorp 96-well plates (ThermoFisher cat no. 12-565-136) were coated with 0.2 μg/100 μL/well human CD73 (Acro Biosciences cat. no CD3-H52H7) in carbonate buffer (MilliporeSigma cat no. C3041). Plates were incubated under static conditions at 4° C. overnight and washed 3× with 300 μL/well 1×PBS pH 7.4 (Fisher Scientific cat no. MT21040CM) supplemented with 0.05% Tween 20 (Fisher Scientific cat no. BP337-500) (PBST) and blocked with 200 μL/well 1% BSA (MilliporeSigma cat no. A5611) in TBS (Teknova T9867) supplemented with 0.05% Tween 20 (TBST) for 1 h at room temperature with shaking (400 rpm). After discarding the blocking solution, duplicate three-fold serial dilutions of the plasma samples were plated at 100 μL/well and incubated at room temperature for 2 h with shaking (sample diluent: 0.5% BSA in TBS 0.025% Tween 20+naïve mouse plasma at final concentration of 1:100). Five-fold dilutions of DFC standard curves ranging from 0.0064 ng/mL-500 ng/mL were run on each plate in duplicate. Following the 2 h incubation, plates were washed 3× with 300 μL/well PBST. Conjugate bound to CD73 on the plates was then probed with a HRP conjugated anti-human IgG Fc F(ab′)₂ (Jackson ImmunoResearch cat no. 709-036-098) diluted 1:2,000 in sample diluent (0.5% BSA in TBS 0.025% Tween 20) for 1 h at room temperature, with shaking and protected from light. Plates were then washed 3× with 300 μL/well PBST and developed with 100 μL/well TMB substrate (BD Biosciences cat no. 555214) for 7-8 min. The reaction was stopped with 100 μL/well 1N H₂SO₄. Absorbance was read at 450 nm with an EnSpire multimode plate reader (PerkinElmer). The concentration of DFC in plasma samples was interpolated using nonlinear regression analysis (Sigmoidal, 4 PL analysis) of the standard curves in GraphPad Prism (Version 8).

The Fc-capture ELISA was performed as described with the following modifications. Nunc Maxisorp 96-well plates were coated overnight at 4° C. under static conditions with 0.1 μg/100 μL/well of goat anti-human IgG (Fcγ fragment specific; Jackson ImmunoResearch cat no. 109-005-098) in carbonate buffer (MilliporeSigma cat no. C3041). Plates were washed 3× with 300 μL/well PBST and blocked with 200 μL/well in blocking solution (TBS+0.05% Tween 20+5% non-fat dry milk (Cell Signaling Technology cat no. 9999S) for 1 h at room temperature with shaking (400 rpm). After discarding the blocking solution, three-fold serial dilutions of the plasma samples were plated at 100 μL/well and incubated at room temperature for 2 h with shaking (sample diluent: 2.5% non-fat dry milk in TBS 0.025% Tween 20+naïve mouse plasma final concentration of 1:900). DFC standard curves ranging from 0.03 ng/mL to 55 ng/mL were run on each plate in duplicate. Following the 2 h incubation, plates were washed 3× with 300 μL/well PBST. Conjugate bound to Fc on the plates was then probed with 100 μL/well of HRP conjugated anti-human IgG Fc F(ab′)₂ (Jackson ImmunoResearch cat no. 709-036-098) diluted 1:2,000 in sample diluent (2.5% non-fat dry milk in TBS 0.025% Tween 20) for 1 h at room temp, with shaking and protected from light. Plates were then washed 3× with 300 μL/well PBST and developed with 100 μL/well TMB substrate reagent for 7-8 min. The reaction was stopped with 100 μL/well 1 N H₂SO₄ and the absorbance read at 450 nm with an EnSpire multimode plate reader (PerkinElmer). Concentration of DFC in plasma samples was interpolated using non-linear regression analysis (Sigmoidal, 4 PL analysis) of the standard curves in GraphPad Prism (Version 8).

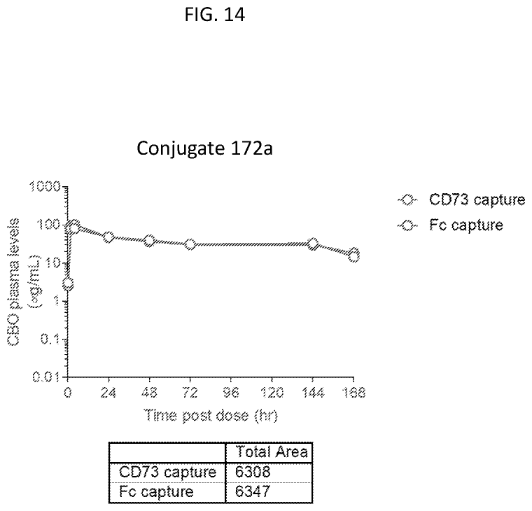

The 7-day mouse PK profiles for Conjugate 172a showed similar drug exposure levels by CD73 capture/Fc detection and Fc capture/Fc detection, with AUCs of 6,308 and 6,347 respectively (FIG. 14). These data demonstrate that Conjugate 172a is a stable small molecule Fc conjugate that is likely eliminated through lysosomal degradation of the intact molecule. The small molecule payload on the circulating material remains conjugated to the protein carrier are likely untransformed through the one week duration of the study. These results highlight the long half-life, high plasma exposures and overall stability of Conjugate 172a in vivo.

Conjugate 133a mouse tumor efficacy study: Mouse syngeneic model with a colon tumor cell line

Tumor efficacy models were conducted in female BALB/c mice (Charles River; 18-22 g) injected with the colon tumor cell line CT26 (ATCC; CRL-2638). Briefly, CT26 cells freshly prepared and resuspended in PBS were injected SC into the shaved right flank of mice. Tumors were allowed to grow until they became measurable (˜50 mm³) (typically after 7-10 days of growth). Mice were then randomized so each starting group had approximately the same tumor group average (typical+/−5 mm³) and treatment initiated. Each group consisted of 10 animals. Conjugate 133a was dosed at 20 mg/kg via intraperitoneal (IP) injection on MWF, for 2 weeks. The small molecule CD73 inhibitor (AB680) was dosed at 30 mg/kg IP daily for 2 weeks. The study also included a vehicle (PBS) only treatment group with the aforementioned schedule. After initiation of treatment tumors were measured 3×/week with tumor volumes calculated based on the following formula: (width×width×length)/2. All tumors were monitored until the maximum allowed volume was reached (=>2,000 mm³).

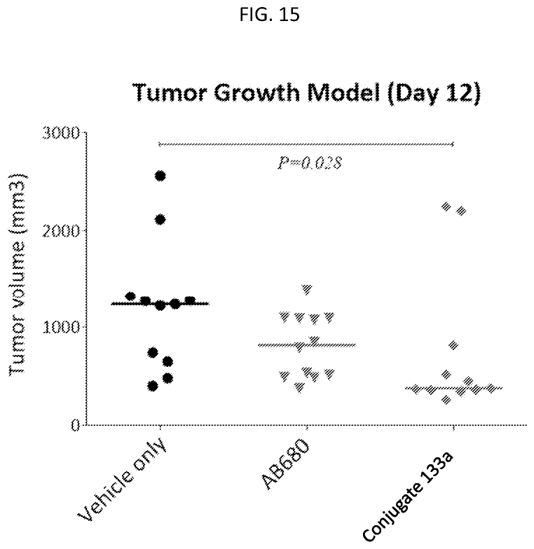

A scatter plot of individual animals on day 14 post-treatment (N=11-12) if provided in FIG. 15. The results of this study show that Conjugate 133a was able to produce a greater mean reduction in tumor volume than AB680 when dosed at half the frequency and, on a molar basis, at approximately 1/150^{th} the dose.

Conjugate 133b Mouse Tumor Efficacy Study: Mouse Syngeneic Model with a Colon Tumor Cell Line and Combination Therapy with Anti-PD-1 Antibody

Tumor efficacy models were conducted in female BALB/c mice (Charles River; 18-22 g) injected with the colon tumor cell line CT26 (ATCC; CRL-2638). Briefly, CT26 cells freshly prepared and resuspended in PBS were injected SC into the shaved right flank of mice. Tumors were allowed to grow until they became measurable (˜50 mm³) (typically after 7-10 days of growth). Mice were then randomized so each starting group had approximately the same tumor group average (typical+/−5 mm³) and treatment initiated. Each group consisted of 10 animals. Conjugate 133b was dosed at 5 and 20 mg/kg via intraperitoneal (IP) injection on MWF, for 2 weeks. The study also included a vehicle (PBS) only treatment group and a group receiving in combination Conjugate 133b (20 mg/kg) and an anti-PD-1 antibody (RMP1-14) with the aforementioned schedule. After initiation of treatment tumors were measured 3×/week with tumor volumes calculated based on the following formula: (width×width×length)/2. All tumors were monitored until the maximum allowed volume was reached (=>2,000 mm³).

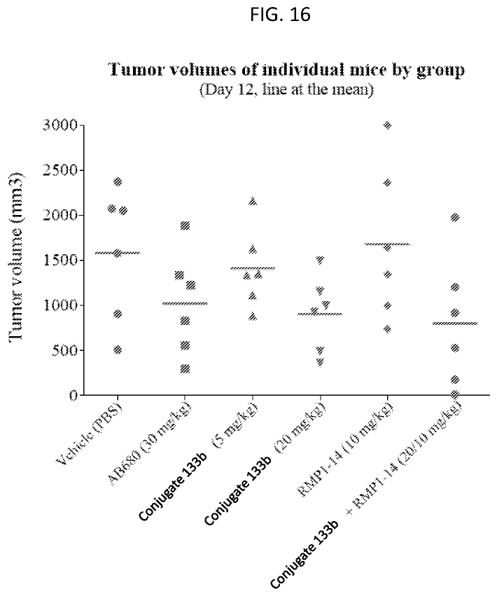

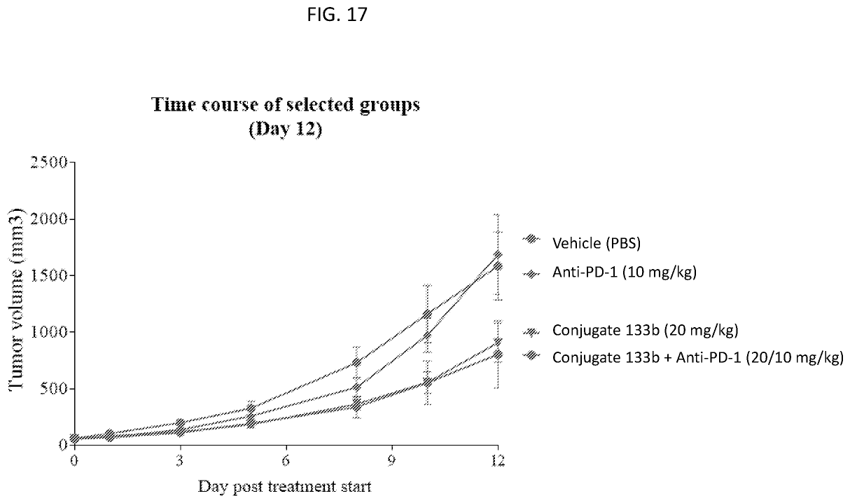

Conjugate 133b demonstrated a dose response with an 11% reduction at the 5 mg/kg dose. When the dose was increased to 20 mg/kg the reduction in tumor volume increased to 43%. The anti-PD-1 mAb RMP1-14 showed no activity as monotherapy. However, in combination with Conjugate 133b there was an increase in tumor reduction (49% for the combo) (FIG. 16). Notably, one mouse in this group demonstrated complete remission. A 12-day time course is provided in FIG. 17.

### Efficacy Study of Conjugate 133b and Conjugate 161 Against a Colon Tumor Cell Line (CT26) in a Syngeneic Mouse Model

Tumor efficacy models were conducted in female BALB/c mice (Charles River; 18-22 g) injected with the colon tumor cell line CT26 (ATCC; CRL-2638). Briefly, CT26 cells freshly prepared and resuspended in PBS were injected SC into the shaved right flank of mice. Tumors were allowed to grow until they became measurable (˜25-50 mm³) (typically after 7-10 days of in vivo growth). Mice were then randomized so each starting group had approximately the same tumor group average (typically +/−5 mm³) and treatment initiated. Group size for all studies was typically 8-12 mice, although occasionally an animal would be removed from study if the tumor grew internally causing undo stress to the animal, or the inability to measure the tumor accurately. Conjugate 133b was typically dosed at 20 mg/kg via intraperitoneal (IP) injection on MWF, for 2 weeks. However, in a study to investigate dosing frequency, dosing was performed 1, 2, or 3× weekly. The small molecule CD73 inhibitor (AB680) was dosed at 30 mg/kg IP daily for 2 weeks. Studies also included vehicle (PBS) only treatment groups. After initiation of treatment tumors were measured 3×/week with tumor volumes calculated based on the following formula: (width×width×length)/2. All tumors were monitored until the maximum allowed volume was reached (=>2,000 mm³). Data was analyzed and graphed with Graphpad Prism 6.00. Where conducted, statistical evaluation used the Mann-Whitney variation of the t-test.

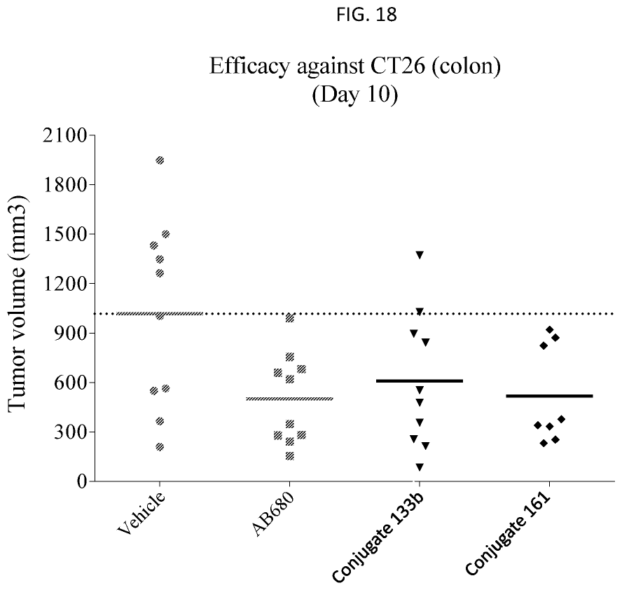

On Day 10 the first animal in the vehicle treated group reached the maximum allowed tumor volume under our IACUC authorization (2,000 mm³). The mean tumor volume for vehicle treated mice was 1,1018 mm³ and is shown in a column graph (FIG. 18). The positive control, AB680 achieved a 50.7% reduction in tumor volume compared to the vehicle group when dosed at 210 mg/kg/wk. In contrast, Conjugate 133b nearly achieved the same level of reduction (40.2%) with a dose of only 60 mg/kg/wk. A related molecule (Conjugate 161) demonstrated an even greater reduction (49.5%) with the same dose schedule as Conjugate 133b.

This study confirmed prior results demonstrating a notably reduction in tumor volume with a modest dose (relative to the positive control) of Conjugate 133b. The results with Conjugate 161 indicate that greater potency is possible with refinement of the Conjugate 133b core.

### Efficacy Study of Conjugate 133b, Conjugate 161, Conjugate 169, Conjugate 165, Conjugate 172a, and Conjugate 175 Against a Colon Tumor Cell Line (CT26) in a Syngeneic Mouse Model.

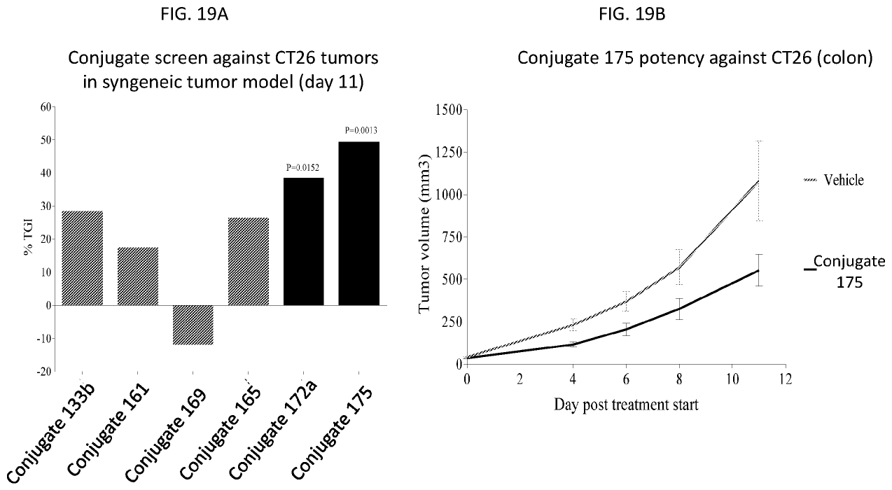

In a subsequent study using the same experimental protocol outlined in “Efficacy study of Conjugate 133b against a colon tumor cell line (CT26) in a syngeneic mouse model” several additional drug-Fc conjugates were screened for activity in a colon (CT26) syngeneic model, except that the Conjugate 172a dose group only received a single dose at 20 mg/kg, instead of the 6 doses the other groups received due to limited compound. FIG. 19A is a plot showing the percent Tumor Growth Inhibition (TGI) relative to the vehicle control. In this study Conjugate 133b is somewhat more active than Conjugate 161 but both are in the 15-30% reduction in overall tumor volume range. Conjugate 165 was also within this same range (25.9% reduction), while Conjugate 169 was inactive in this study with a slightly higher tumor volume than vehicle treated animals; this was within the experimental error typical in this model.

In this study Conjugate 172a and Conjugate 175 were the most active drug-Fc conjugates with TGI's of 37.9 and 48.8%, respectively (FIG. 19A), which were statistically significant (P=0.0152 and 0.0013, respectively). The activity of Conjugate 172a is even more notable considering that this group received a total dose over the course of the study of 20 mg/kg, compared to the other groups which received a cumulative dose of 120 mg/kg. The observation that Conjugate 175 achieved close to a 50% reduction in tumor size supports the importance of CD73 in our CT26 screening model, and CD73's significance as a therapeutic target for cancer treatment. FIG. 19B is a line graph of tumor volume in Conjugate 175 versus vehicle treated animals. This graph illustrates the statistically significant difference in tumor volume from Day 4 through Day 11 for this conjugate.

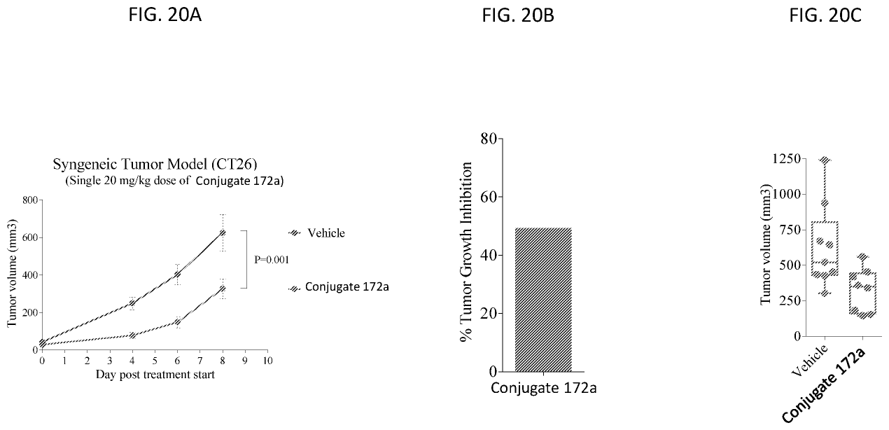

Since Conjugate 172a was only administered as a single dose, compared to multiple doses for the other conjugates in this study, we also evaluated efficacy at Day 8 for this DFC. This was done because the activity of Conjugate 172a appeared to tail off slightly between Day 8 and 11, likely because the concentration of Conjugate 172a fell below its optimal concentration. Through Day 8 Conjugate 172a displayed strong anti-tumor effects relative to the vehicle control (FIG. 20A). This difference was pronounced and statistically significant over the first 8 days of the study with a P value of 0.001.

On Day 8 the vehicle group had an average tumor volume of 625 mm³, in contrast, the tumor volume of mice administered a single 20 mg/kg dose of Conjugate 172a was only 326 mm³. This equates to a 49% tumor growth inhibition value (FIG. 20B). Considering that this reduction resulted from a single dose, compared to 6 doses for the other conjugates in this study, illustrates the significant potency of Conjugate 172a to suppress an aggressive colon tumor in a syngeneic mouse model. In addition to a reduction in the median for the Conjugate 172a treated group, the spread in tumor volumes is greatly reduced. Importantly, this difference was achieved with a single 20 mg/kg dose of DFC (FIG. 20C).

### Pharmacokinetics (PK) of Conjugate 172a in Mice

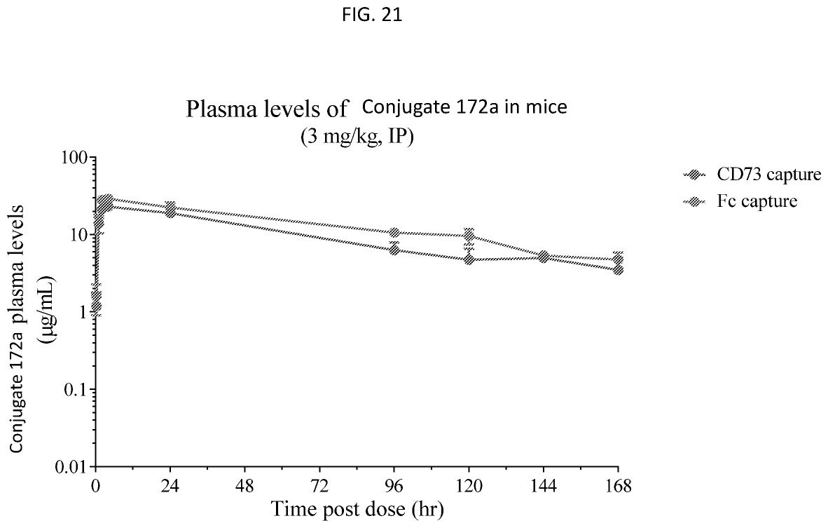

The PK of Conjugate 172a was determined in BALB/c mice (n=3). In this study mice were administered a single IP injection at 3 mg/kg. Mice were bled periodically for one week and plasma was isolated by centrifugation. Over one week, Conjugate 172a levels remained fairly constant, consistent with a long circulating half-life in mice (FIG. 21). To determine the stability of Conjugate 172a in vivo, dual-capture sandwich ELISA methods were used. Briefly, side-by-side assays were conducted using either CD73 or Fc as the primary capture method, followed by Fc detection. Comparing the AUC ratios from both capture methods allows for evaluation of retention of functional conjugated small molecule inhibitors on the conjugate. In this study the AUC for the CD73 capture analysis was 2952, versus 2528 for the Fc capture method. The nearly identical exposures measured using the two capture methods demonstrate that circulating Conjugate 172a remains intact and unchanged.

### Dose Schedule Study of Conjugate 133b Against a Colon Tumor Cell Line (CT26) in a Syngeneic Mouse Model.

The effect of different dosing schedules on the efficacy of Conjugate 133b was investigated in a dose schedule study. This study used the general protocol detailed in “Efficacy study of Conjugate 133b against a colon tumor cell line (CT26) in a syngeneic mouse model” except Conjugate 133b was dosed at 20 mg/kg once, twice, or 3-times weekly. This study also included an anti-CTLA-4 mAb as a positive control (Ichorbio, cat. ICH1084) dosed at 10 mg/kg on Day 0, 3, 6, and 9. CTLA-4 is a related immune checkpoint in the adenosinergic axis but occurs at an earlier stage of T-cell activation than CD73.

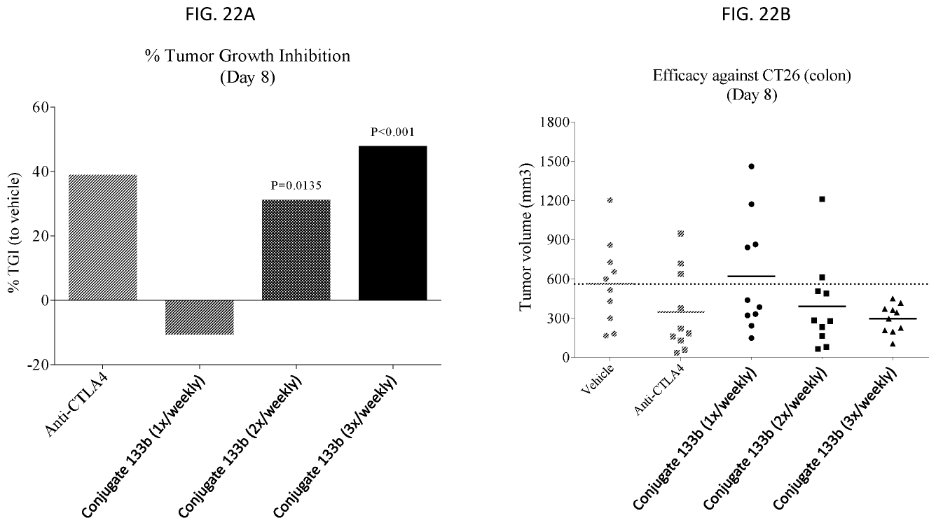

In this study a single dose of Conjugate 133b was no more efficacious than vehicle treated animals (FIG. 22A). However, when dosed twice, or 3-times weekly Conjugate 133b demonstrated significant efficacy relative to vehicle treated mice. With two weekly doses there was a 30.5% reduction in tumor volume (P=0.0135). This was increased to 47.3% (P<0.001) when Conjugate 133b was dosed 3-times weekly. This study demonstrated a clear dose-dependency with Conjugate 133b, and greater activity than the control antibody when dosed 3-times per week. Also notable is the tight clustering of tumor volumes with 3 weekly doses, and the absence of poorly responding animals evident with once or twice weekly dosing (FIG. 22B).

### Tolerability Study of Conjugate 172a in Mice (IV Dosing).

The IV tolerability of Conjugate 172a was investigated in a small, two mouse per group study using female Balb/c mice (6-8 weeks old). Control mice were administered vehicle (20 mM Histidine, 7% Sucrose, 0.02% PS80, pH 5.5); Conjugate 172a was dosed at 125, 250, 500, or 1000 mg/kg. Additionally, one mouse in the 1000 mg/kg dose group was given a second dose on Day 6 at 250 mg/kg for a cumulative dose of 1250 mg/kg over the course of the study (12 days). IV administration was by a slow push of approximately 45 seconds duration. Clinical symptoms were monitored twice daily, and body weight (BW) recorded once daily. At study end a gross necropsy was performed on all study animals.

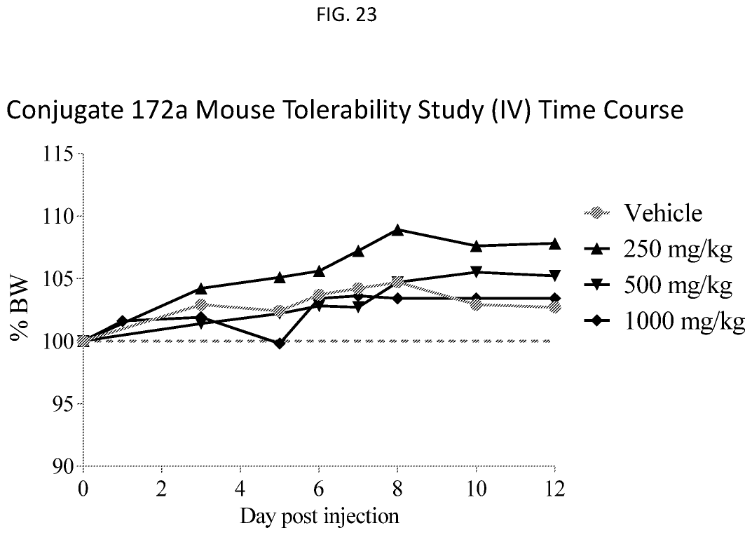

Conjugate 172a was exceedingly well tolerated, with no clinical symptoms noted for any dose group throughout the study. As expected, no symptoms were seen in the vehicle group. BWs are graphed in FIG. 23 and no significant weight loss was seen throughout the study with all animals exceeding their starting BW at study end. A gross necropsy was performed on Day 12 of all study animals examining all major organs for signs of abnormalities. No abnormal organs/tissues findings were noted.

Lastly, the single 1000 mg/kg animal that received a second dose of Conjugate 172a at 250 mg/kg on Day 6 was indistinguishable by all study criteria as its cage-mate which received a single dose of compound. Collectively, no adverse effects were found with Conjugate 172a at very high concentrations (1000 mg/kg as a single IV dose).

### PBMC Activation Assay by Flow Cytometry Using Conjugate 172c

Human PBMCs (StemCell Technologies cat no. 70025) prepared in ImmunoCult™—XF T cell Expansion medium (StemCell Technologies cat no. 10981) were seeded at 1×10⁵ cells per well (100 μL of 1×10⁶ cells/mL) in the presence or absence of T cell activator anti-CD3/CD28/CD2 (StemCell Technologies cat no. 10970) in 96 well round bottom plates (Corning cat no. 3788). Ten-fold serial dilutions of each test article were prepared, resulting in final concentrations ranging from 0.1 nM-10,000 nM. 50 μL of test article was added to the cells, followed by 50 μL of adenosine 5′ monophosphate (AMP; MilliporeSigma cat no. A1752) for a final concentration of 30 μM. Plates were incubated at 37° C. with 5% CO₂ for 72 h. After 72 h the cells were centrifuged for 5 min at 300×g at 4° C. then washed twice with cell staining buffer (BioLegend cat no. 420201). Cells were incubated with Fc block (Unlabeled, Clone: 3070; BD Biosciences cat no. 5642220) for 10 min at 4° C. Cells were stained with anti-human CD25 antibody, clone 2A3, PE (StemCell Technologies cat no. 60153PE) for 20 min at 4° C. protected from light. Cells were washed twice with cell staining buffer and resuspended in 200 μL of cell staining buffer. Samples were acquired using a Cytek Northern Lights flow cytometry system. CD25⁺ by flow cytometry was used as the readout to measure PBMC activation after AMP inhibition. Samples were analyzed using FlowJo version 10. Percent rescue (x-axis) versus inhibitor concentration (y-axis) was plotted using GraphPad Prism Version 6 and EC₅₀ values were generated using nonlinear regression analysis.

In order to assess the impact of DAR on in vitro potency, six batches of Conjugate 172c were tested to determine their ability to re-activate PBMCs inhibited by 30 μM AMP. The DARs of the batches tested ranged from 5.7-13.3. All batches of Conjugate 172c demonstrated potent activity (average EC₅₀ value of 21.6 nM) in the presence of 30 μM AMP, except batch 2, which displayed moderate activity (EC₅₀=170 nM). This was expected due to the low DAR of batch 2.

OP5244 was included as a control and showed potent activity, with an EC₅₀ value of 17 nM, (Table 13).

TABLE 13
Activity of various batches of Conjugate 172c targeting CD73
in a PBMC activation assay using CD25+ as a read-out.

| Test article | DAR | 30 μM AMP EC₅₀ [nM] |
|---|---|---|
| Conjugate 172c (batch 2) | 5.7 | 170 |
| Conjugate 172c (batch 1) | 7.5 | 25 |
| Conjugate 172c (batch 3) | 8.2 | 28 |
| Conjugate 172c (batch 4) | 10 | 26 |
| Conjugate 172c (batch 5) | 11.3 | 16 |
| Conjugate 172c (batch 6) | 13.3 | 13 |
| OP-5244 | N/A | 17 |

### Cell-Based and Cell-Free CD73 Enzyme Inhibition Assays Using Conjugate 172c

The cell-free CD73 enzyme inhibition assay was performed in a 96-well plate format using the CD73 Inhibitor Screening Assay Kit in accordance with the manufacturer's instructions (BPS Bioscience cat no. 72055). Briefly, test inhibitors and the positive control (AB680; Lawson et al., 2020) were incubated in assay buffer (6 μL/well) in the presence of AMP (10 μL/well, final 100 μM) and recombinant human CD73 enzyme (0.1 ng/μL, 20 μL/well) for 25 min at 37° C. Colorimetric detection reagent was then added (100 μL/well) and the free phosphate from the CD73 reaction was measured by reading the absorbance at 630 nm using the EnSpire multimode plate reader (PerkinElmer). Wells without test inhibitor (CD73 positive control) were included on each plate. Blank wells without test inhibitor and CD73 were subtracted from all wells. Percent CD73 inhibition (y-axis) versus log₁₀ inhibitor concentration (x-axis) was plotted using GraphPad Prism Version 8 and IC₅₀'s calculated using nonlinear regression analysis (Sigmoidal, 4 PL).

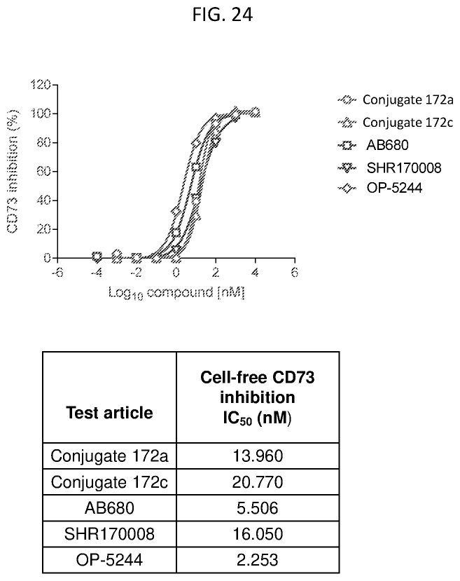

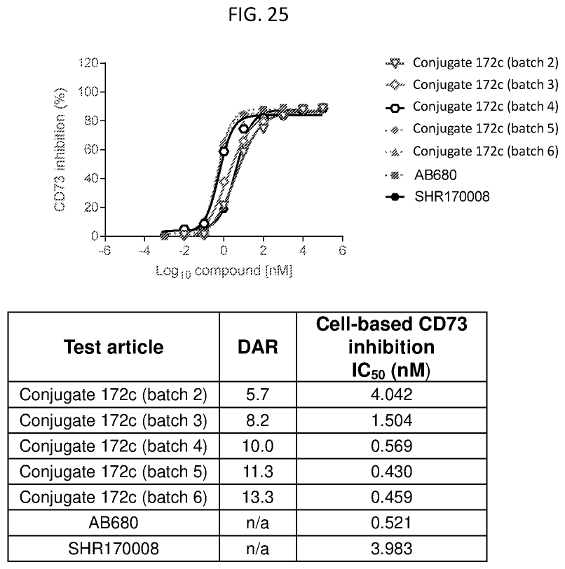

To demonstrate equipotency between Conjugate 172a and Conjugate 172c, Conjugate 172a was tested along with Conjugate 172c and small molecule CD73 inhibitors (AB680, SHR170008 and OP-5244) in the cell-free CD73 enzyme inhibition assay (FIG. 21). Conjugate 172c (IC₅₀20.77 nM) showed a potency within 2-fold of Conjugate 172a (IC₅₀ 13.96 nM), demonstrating that Conjugate 172c was equipotent to Conjugate 172a in this assay (FIG. 24).

The cell-based CD73 enzyme inhibition assay was performed essentially as described above for the cell-free assay, with the following modifications. MDA-MB-231 human breast adenocarcinoma cells (ATCC CRM-HTB-26) were seeded at 2×10⁴ cells/well/100 μL into 96-well tissue culture-treated plates (Corning Costar cat no. 3596) and incubated overnight at 37° C., 5% CO₂. Cells were incubated with test inhibitors (50 μL/well) and AMP (25 μL/well, final 400 μM) for 3 h at 37° C., 5% CO₂ to allow for interaction with CD73 expressed on the MDA-MB-231 cell surface. The 96-well plate was then centrifuged at 125×g for 5 min at room temperature and ATP (25 μL/well, 100 μM final) was combined with supernatant (25 μL/well) in a white opaque 96-well plate (Corning Costar cat no. 3912). The CD73 activity in the supernatant was quantified using the CellTiter-Glo kit (Promega cat no. G7571) as previously described (Sachsenmeier et al., 2012). Percent CD73 inhibition (y-axis) versus log₁₀ inhibitor concentration (x-axis) were plotted using GraphPad Prism Version 8 and IC₅₀'s calculated using nonlinear regression analysis (Sigmoidal, 4 PL).

A DAR scan was performed in the cell-based assay to explore the relationship between DAR and CD73 enzyme inhibition potency. Conjugate 172c (batch 2) (DAR 5.7), Conjugate 172c (batch 3) (DAR 8.2), Conjugate 172c (batch 4) (DAR 10.0), Conjugate 172c (batch 5) (DAR 11.3), and Conjugate 172c (batch 6) (DAR 13.3) were tested alongside the small molecule benchmark inhibitors AB680 and SHR170008. Conjugate 172c (batch 2) with DAR 5.7 (IC₅₀ 4.042 nM) had decreased in potency when compared to the higher DAR conjugates. Conjugate 172c (batch 3) with DAR 8.2 (IC₅₀ 1.504 nM), Conjugate 172c (batch 4) with DAR 10.0 (IC₅₀ 0.569 nM), Conjugate 172c (batch 5) with DAR 11.3 (IC₅₀ 0.430 nM), and Conjugate 172c (batch 6) with DAR 13.3 (IC₅₀ 0.459 nM) were all within 1-3 fold potency of each other and of the small molecule benchmark inhibitor AB680 (IC₅₀ 0.521 nM) (FIG. 24).

### Human CD73 Binding by Surface Plasmon Resonance

Kinetic binding of drug-Fc conjugates (DFCs) and comparator monoclonal antibodies (mAbs) to human CD73 by surface plasmon resonance (SPR) was performed by Acro Biosystems using the Biacore 8 k system (Cytiva). The anti-histidine antibody included in the His Capture Kit, type 2 (cat no. 29234602, Cytiva) was diluted to 50 μg/mL in immobilization buffer (10 mM sodium acetate, pH 4.5; cat no. 29234602, Cytiva) and immobilized at a flow rate of 10 μL/min for 420 s to 10,000 RU on a Series S Sensor Chip CM5 (cat no. 29149603, Cytiva). Enzymatically active His-tagged human CD73/NT5E protein (cat #CD3-H52H7, Acro Biosystems) was diluted to 1 μg/mL in 1×HBS-EP running buffer (10 mM HEPES, 150 mM NaCl, 3 mM EDTA and 0.5% v/v Surfactant P20, pH 7.4) and captured at a flow rate 10 μL/min to 30 RU. Sensorgrams were recorded by flowing 2-fold dilutions of analyte (DFC or mAb) in running buffer at concentrations ranging from 125 nM to 0.977 nM. Binding was measured at a flow rate of 30 μL/min, with a 90 s association phase and 120 s dissociation phase. The sensor chip surface was regenerated between each run by a 60 s injection of 10 mM glycine-HCl, pH 1.5 (cat no. 29234602, Cytiva) at a flow rate of 30 μL/min. Binding kinetics were analyzed using the data analysis software. Steady state affinity or 1:1 Langmuir binding models were utilized to fit the data.

Binding of small molecule benchmark inhibitors to human CD73 by SPR was performed by Acro Biosystems using the Biacore 8 k system (Cytiva). Enzymatically active His-tagged human CD73/NT5E protein (cat #CD3-H52H7, Acro Biosystems) was diluted to 30 μg/mL in 1×PBS-P running buffer (2 mM KH₂PO₄, 10 mM Na2HPO₄, 137 mM NaCl, 2.7 mM KCl with 0.05% Tween-20, pH 7.4), 50 μM EDTA, 5% DMSO) included in the NTA Reagent Kit (cat no. 28995043, Cytiva) and captured at a flow rate 10 μL/min to 3,000 RU on a Series S Sensor Chip NTA (cat no. BR100532, Cytiva). Sensorgrams were recorded by flowing 2-fold dilutions of DFC analytes in running buffer at concentrations ranging from 15.625 nM to 0.977 nM. Binding was measured at a flow rate of 30 μL/min, with a 120 s association phase and 300 s dissociation phase. The sensor chip surface was regenerated between each run by a 60 s injection of 350 mM EDTA (cat no. 28995043, Cytiva) at a flow rate of 30 μL/min. Binding kinetics were analyzed using the data analysis software. A 1:1 Langmuir binding model was utilized to fit the data.

The DFCs screened in this study had similar K_{D}'s for human CD73 binding, ranging from 13.7 to 26.9 nM (Table 14). Compared to Mupadolimab (K_{D}=123 nM), the DFCs bound CD73 with 4.5 to 8.9-fold greater affinity. Oleclumab (K_{D}=0.527) demonstrated 25.9 to 51.0-fold greater binding affinity to CD73, compared to the DFCs. The small molecule benchmarks AB680 (K_{D}=2.64 nM), OP-5244 (K_{D}=0.523 nM) and SHR170008 (K_{D}=3.34 nM) had 4.1 to 51.4-fold greater affinity for CD73, compared to the DFCs. The hlgG1 Fc negative control did not bind CD73, as expected (Table 14).

TABLE 14
SPR equilibrium dissociation constants (K_{D}) for the binding
of DFCs, anti-CD73 mAbs and small molecule CD73 inhibitors
to enzymatically active human CD73 protein.

| | Drug-to-antibody | K_{D} |
|---|---|---|
| Test article | ratio (DAR) | (nM) |
| hIgG1 Fc (negative control) | n/a | No binding |
| Conjugate 133b | 7.7 | 16.7 |
| Conjugate 175 | 6.8 | 14.8 |
| Conjugate 191 | 5.8 | 16.0 |
| Conjugate 165 | 8.1 | 15.3 |
| Conjugate 175 | 4.7 | 26.9 |
| Conjugate 178 | 8.1 | 23.7 |
| Conjugate 181 | 5.7 | 13.7 |
| Conjugate 183 | 6.7 | 26.3 |
| Conjugate 172c | 7.5 | 19.0 |
| Oleclumab | n/a | 0.527 |
| Mupadolimab | n/a | 123 |
| AB680 | n/a | 2.64 |
| OP-5244 | n/a | 0.523 |
| SHR170008 | n/a | 3.34 |

### Human CD73 Binding by Surface Plasmon Resonance (Conjugate 172c (Batch 3) and Conjugate 190)

The kinetic binding of drug-Fc conjugates (DFCs) and comparator monoclonal antibody (mAb) Oleclumab to human CD73 by surface plasmon resonance (SPR) was performed by Acro Biosystems using the Biacore 8 k system (Cytiva). The anti-histidine antibody included in the His Capture Kit, type 2 (cat no. 29234602, Cytiva) was diluted to 50 μg/mL in immobilization buffer (10 mM sodium acetate, pH 4.5; cat no. 29234602, Cytiva) and immobilized at a flow rate of 10 μL/min for 420 s to 10,000 RU on a Series S Sensor Chip CM5 (cat no. 29149603, Cytiva). Enzymatically active His-tagged human CD73/NT5E protein (cat #CD3-H52H7, Acro Biosystems) was diluted to 1 μg/mL in 1×HBS-EP running buffer (10 mM HEPES, 150 mM NaCl, 3 mM EDTA and 0.5% v/v Surfactant P20, pH 7.4) and captured at a flow rate 10 μL/min to 30 RU. Sensorgrams were recorded by flowing 2-fold dilutions of analyte (DFC or mAb) in running buffer at concentrations ranging from 125 nM to 0.977 nM. Binding was measured at a flow rate of 30 μL/min, with a 90 s association phase and 120 s dissociation phase. The sensor chip surface was regenerated between each run by a 60 s injection of 10 mM glycine-HCl, pH 1.5 (cat no. 29234602, Cytiva) at a flow rate of 30 μL/min. Binding kinetics were analyzed using the data analysis software. The 1:1 Langmuir binding model was used to fit the data.

The DFCs screened in this study, Conjugate 172c (batch 3) and Conjugate 190, displayed similar binding affinity for human CD73, with K_{D}'s of 1.05 nM and 1.42 nM, respectively (Table 15). The anti-CD73 mAb Oleclumab (K_{D}=0.109 nM) demonstrated 9.6 to 13.0-fold greater binding affinity to CD73, compared to the DFCs.

TABLE 15
SPR equilibrium dissociation constants (K_{D}) for the
binding of Conjugate 172c (batch 3), Conjugate 190,
and Oleclumab to enzymatically active human CD73.

| Test article | Drug-to-antibody ratio (DAR) | K_{D} (nM) |
|---|---|---|
| Conjugate 172c (batch 3) | 8.2 | 1.05 |
| Conjugate 190 | 7.0 | 1.42 |
| Oleclumab | n/a | 0.109 |

### CD73 Binding Assay by Flow Cytometry

The binding of drug-Fc conjugates (DFCs) to the MDA-MB-231 (ATCC CRM-HTB-26) cancer cell line was determined using the following steps. Cancer cells were washed twice with PBS (Gibco cat. no 10010023), detached from the tissue culture flask via cell scraper (Fisher cat no. 08-771-1B) and strained using a 70 μm cell strainer (StemCell Technologies cat. no 27260). Cells were seeded in 100 μL cell staining buffer (BioLegend cat no. 420801) at 1×10⁵ cells per well in a nontreated 96 well round bottom plate (Corning cat. no 3788). Serial dilutions of DFCs from 0.00004 nM-4,000 nM (4× final concentration) at 50 μL/well in the presence or absence of small molecule CD73 inhibitor AB680 at 4, 40, 400 nM (4× final concentration) or adenosine monophosphate (AMP; Sigma-Aldrich cat. no A9396) at 40, 400 or 4,000 μM (4× final concentration), respectively. Cells and test articles were incubated with for 37° C. with 5% CO₂ for 60 min. Cells were then centrifuged at 300×g for 3 min at room temperature and the supernatant was discarded. Cells were washed twice at 300×g for 3 min. Cells were stained with a polyclonal anti-human Fc antibody (Invitrogen cat. no 12-4998-82) at 1 μL per 100 μL/well. Cells were resuspended and incubated for 15-20 min at 4° C. protected from light. Following the incubation, 100 μL/well of cell staining buffer was added and the plate was centrifuged at 300×g for 3 min. The supernatant was discarded, and an additional 200 μL/well of cell staining buffer was added to plate. Cells were resuspended, then centrifuged at 300×g for 5 min. Cells were washed three times at 300×g for 3 min. Samples were acquired on Northern Lights (Cytek) flow cytometer. The gMFI anti-human Fc labeled cells was determined in FlowJo (v.10) and graphed in Prism. The IC₅₀'s for each test article was calculated based on gMFI signal using the 4 PL non-linear regression analysis in GraphPad Prism software.

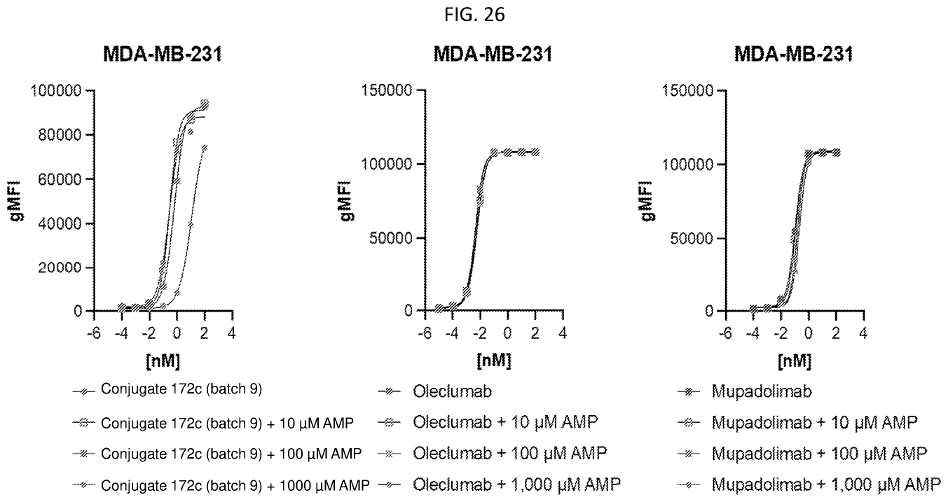

Conjugate 172c (batch 9) binding to MDA-MB-231 cells (IC₅₀ 0.17 nM) was comparable to Mupadolimab (IC₅₀ 0.10 nM) (FIG. 26, Table 16). Oleclumab binding to MDA-MB-231 cells (IC₅₀ 0.0045 nM) was more potent as compared to Conjugate 172c (batch 9). The binding of Conjugate 172c (batch 9) in the presence of AMP was reduced in a dose-dependent manner, demonstrating that Conjugate 172c (batch 9) is an AMP-competitive CD73 inhibitor. Similarly, albeit reduced as compared to Conjugate 172c (batch 9), the binding of Mupadolimab to CD73 was slightly reduced (2-fold) in the presence of high AMP concentration as has been reported in the presence of a non-hydrolyzable AMP analogue (Miller et al., 2022). No difference in binding was observed for Oleclumab in the presence of AMP (FIG. 26 Table 16).

TABLE 16
IC₅₀ of binding of Conjugate 172c (batch 9), Oleclumab and Mupadolimab
in the presence and absence of AMP to human MDA-MB-231 cancer cells.

| | | 0 μM AMP | 10 μM AMP | 100 μM AMP | 1,000 μM AMP |
|---|---|---|---|---|---|
| Test article | DAR | IC₅₀ [nM] | IC₅₀ [nM] | IC₅₀ [nM] | IC₅₀ [nM] |
| Conjugate | 7.9 | 0.17 | 0.21 | 0.52 | 8.61 |
| 172c (batch 9) | | | | | |
| Oleclumab | n/a | 0.0045 | 0.0054 | 0.0059 | 0.0044 |
| Mupadolimab | n/a | 0.10 | 0.12 | 0.16 | 0.20 |

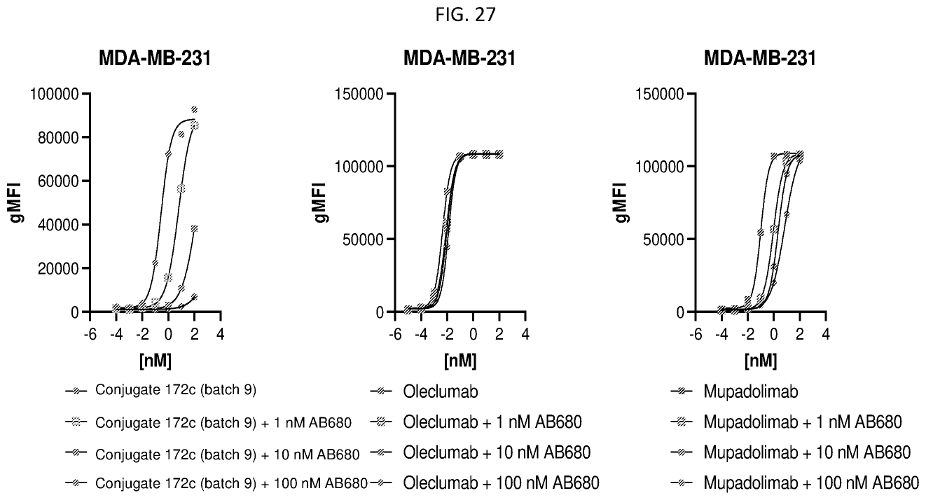

The binding of Conjugate 172c (batch 9) in the presence of small molecule CD73 inhibitor, AB680, was reduced in a dose-dependent manner demonstrating that Conjugate 172c (batch 9) is a catalytic site CD73 inhibitor (FIG. 27, Table 17). Similarly, albeit reduced as compared to Conjugate 172c (batch 9), the binding of Mupadolimab was reduced in a dose-dependent manner in the presence of AB680. No difference in binding was observed for Oleclumab in the presence of AB680. The absence of a change in CD73 binding observed with Oleclumab in the presence of increasing concentrations of AMP or AB680 highlights the difference between mechanisms of action of Oleclumab and Conjugate 172c: Conjugate 172c is an AMP competitive inhibitor, while Oleclumab binds outside of the CD73 active site and does not inhibit catalytic activity via direct competition with substrate.

TABLE 17
IC₅₀ of binding of Conjugate 172c (batch 9), Oleclumab and
Mupadolimab in the presence and absence of small molecule
CD73 inhibitor, AB680, to human MDA-MB-231 cancer cells.

| Test | | 0 nM AB680 | 1 nM AB680 | 10 nM AB680 | 100 nM AB680 |
|---|---|---|---|---|---|
| article | DAR | IC₅₀ [nM] | IC₅₀ [nM] | IC₅₀ [nM] | IC₅₀ [nM] |
| Conjugate 172c | 7.9 | 0.17 | 1.81 | 11.47 | 62.63 |
| (batch 9) | | | | | |
| Oleclumab | n/a | 0.0045 | 0.0084 | 0.0097 | 0.013 |
| Mupadolimab | n/a | 0.10 | 0.88 | 1.54 | 4.19 |

### Cell-Based CD73 Enzyme Inhibition Assay for Mouse EMT6 Cells and Human PBMCs

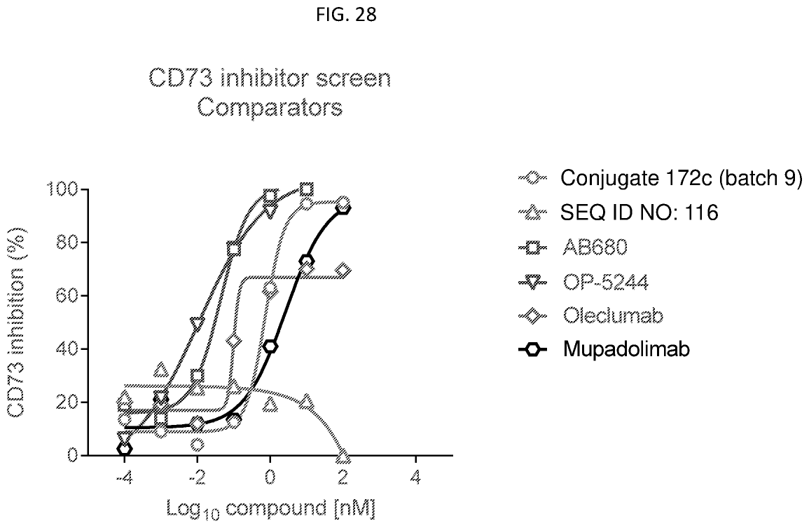

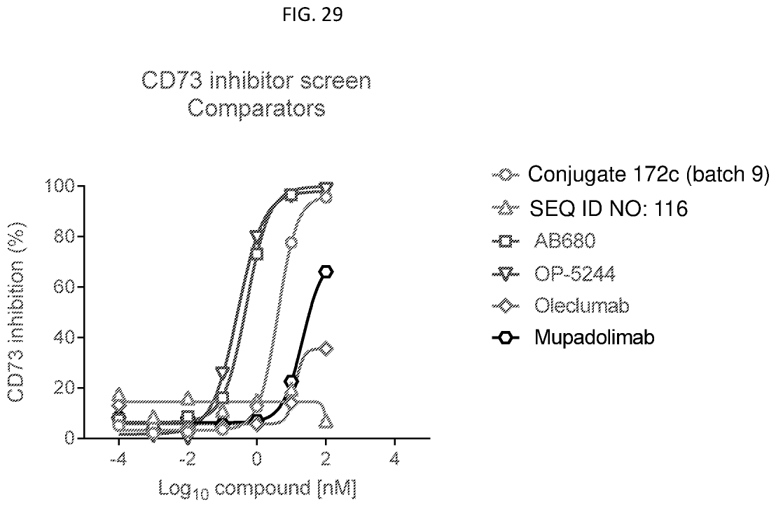

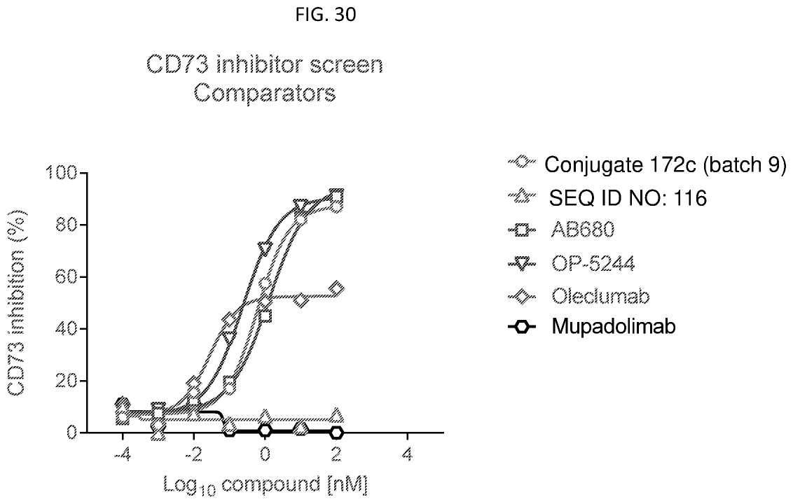

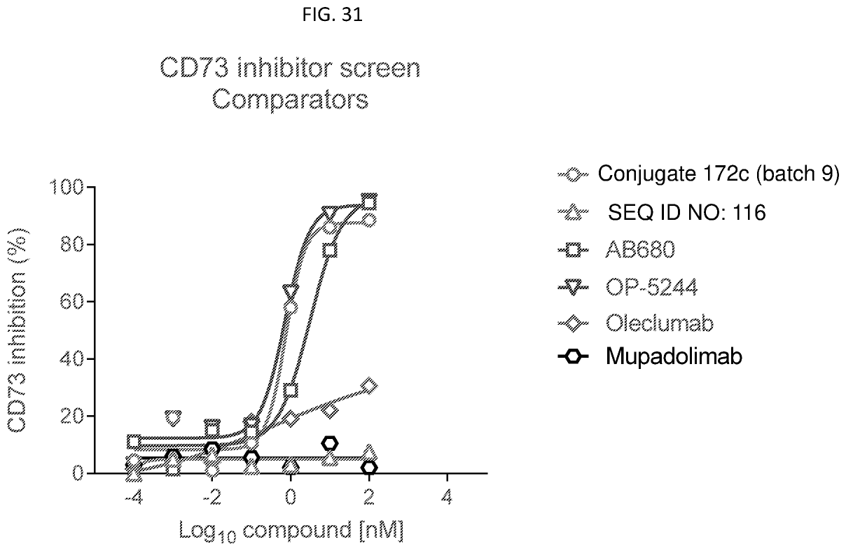

The cell-based CD73 enzyme inhibition assay was performed in a 96-well plate format using the Phosphate Assay Kit—PiColorLock™ in accordance with the manufacturer's instructions (cat no. 270004, Abcam). AMP is enzymatically converted by CD73 to adenosine and phosphate. Therefore, adenosine concentrations can be inferred from phosphate concentrations in 1:1 ratio. Both EMT6 mouse mammary carcinoma cells (ATCC CRL-2755) and human peripheral blood mononuclear cells (PBMC; Stemcell Technologies cat no. 70025, lot no. 2202401007) were assayed. The EMT6 cells were washed three times with a phosphate free CD73 buffer, detached from flasks using a cell scraper, and seeded at 3×10³ cells/well in 100 μL into 96-well tissue culture-treated plates. Human PBMC cells were washed three times by suspending in phosphate free CD73 buffer and pelleting in a centrifuge at 125×g for 10 min at room temperature and seeded at 5×10⁵ cells/well in 100 μL into 96-well tissue culture-treated plates. Test articles, Conjugate 172c (batch 9), SEQ ID NO: 116 (unconjugated Fc control), small molecule CD73 inhibitors AB680, OP5244 or monoclonal antibodies Oleclumab and Mupadolimab were added to cells and incubated for 30 min at 37° C., 5% CO₂. AMP was added at 100 μM and cells, test articles, and AMP were incubated together for 3 h and 24 h at 37° C., 5% CO₂. The CD73 activity in the supernatant was quantified using the PiColorLock Phosphate assay kit. Colorimetric detection reagent was added and the free phosphate from the CD73 reaction was measured by reading the absorbance at 635 nm using the EnSpire multimode plate reader (PerkinElmer). Wells without test inhibitor (CD73 positive control) were included on each plate. Wells without test inhibitor and cells (background) were subtracted from all wells. The percent CD73 inhibition versus inhibitor concentration was plotted using GraphPad Prism Version 8 and IC₅₀'s calculated using nonlinear regression analysis (Sigmoidal, 4 PL). The IC₅₀'s for Conjugate 172c (batch 9) were 0.61 nM and 0.77 nM at 3 h for PBMCs and EMT6 cells, respectively (Tables 18-19, FIGS. 28-31). Compared to other test articles, Conjugate 172c (batch 9) exhibited the smallest fold increase in IC₅₀ at the 24 h mark with IC₅₀'s of 4.07 and 0.83 nM for PBMCs and EMT6 cells, respectively (Tables 18-19). As expected, the unconjugated Fc control did not have any activity against CD73 on human PBMCs or EMT6 cancer cells. The most potent activity was observed for the small molecule CD73 inhibitors against human PBMCs. Conjugate 172c (batch 9) and the small molecule inhibitors had comparable activity against EMT6 cancer cells. The anti-CD73 mAbs had comparable activity to Conjugate 172c (batch 9) at 3 h in PBMCs, but lost >2 logs in activity at 24 h. Additionally, Oleclumab only partially inhibits CD73 at the 3 h time point against human PBMCs and EMT6 cancer cells (FIGS. 28-31). Mupadolimab does not bind murine CD73, that explains the lack of activity in EMT6 cancer cells.

TABLE 18
Activity of Conjugate 172c (batch 9) and comparators targeting
CD73 in a CD73 inhibition assay using human PBMCS.

| Test | 3 h | 24 h |
|---|---|---|
| article | IC₅₀ [nM] | IC₅₀ [nM] |
| Conjugate 172c | 0.61 | 4.07 |
| (batch 9) | | |
| SEQ ID | >100 | >100 |
| NO: 116 | | |
| AB680 | 0.022 | 0.41 |
| OP-5244 | 0.011 | 0.28 |
| Oleclumab | 0.56 | >100 |
| Mupadolimab | 2.85 | 44.16 |

TABLE 19
Activity of Conjugate 172c (batch 9) and comparators targeting
CD73 in a CD73 inhibition assay using mouse EMT6 cancer cells.

| Test | 3 h | 24 h |
|---|---|---|
| article | IC₅₀ [nM] | IC₅₀ [nM] |
| Conjugate 172c | 0.77 | 0.83 |
| (batch 9) | | |
| SEQ ID | >100 | >100 |
| NO: 116 | | |
| AB680 | 1.06 | 2.29 |
| OP-5244 | 0.28 | 0.51 |
| Oleclumab | 5.68 | >100 |
| Mupadolimab | >100 | >100 |

The conversion from AMP to adenosine and phosphate were determined for each time point by comparing the phosphate concentration in the wells of cell only to a phosphate standard curve. AMP conversion to adenosine and phosphate is shown in Table 20.

TABLE 20
Adenosine concentration in the presence of cells at 3 h or 24 h
extrapolated from phosphate standard curve in PiColorLock assay.

| | 3 h | 24 h |
|---|---|---|
| Cell type | phosphate [μM] | phosphate [μM] |
| PBMC Donor 1 | 22.1 | 118.1 |
| EMT6 | 31.6 | 131.9 |

### PBMC Activation Assay Using Conjugate 172c (Batch 9)

Human PBMCs (StemCell Technologies cat no. 70025) prepared in ImmunoCult™—XF T cell Expansion medium (StemCell Technologies cat no. 10981) were seeded at 1×10⁵ cells per well (100 μL of 1×10⁶ cells/mL) in the presence or absence of T cell activator anti-CD3/CD28/CD2 (StemCell Technologies cat no. 10970) in 96 well round bottom plates (Corning cat no. 3788). Ten-fold serial dilutions of each test article was prepared in medium (ranging from 4,000 nM-0.4 nM), resulting in final concentrations ranging from 1,000 nM-0.1 nM. 50 μL of test article was added to the cells, followed by 50 μL of adenosine 5′ monophosphate (AMP; MilliporeSigma cat no. A1752) for a final concentration of 30 μM. Plates were incubated at 37° C. with 5% CO₂ for 72 h. After 72 h the cells were centrifuged for 5 min at 300×g at 4° C. and the supernatant was reserved for analysis of AMP conversion using CellTiter-Glo (Promega cat no. G7571).

Cells were washed twice with cell staining buffer (BioLegend cat no. 420201). Cells were incubated with Fc block (Unlabeled, Clone: 3070; BD Biosciences cat no. 5642220) for 10 min at 4° C. Cells were stained with anti-human CD25 antibody, clone 2A3, PE (StemCell Technologies cat no. 60153PE), anti-human CD4 Antibody, clone SK3 PE/Fire™ 810 (BioLegend cat no 344677), and anti-human CD8 Antibody, clone SK1, PerCP (BioLegend 344708) for 20 min at 4° C. protected from light. Cells were fixed and permeabilized for 20 min using Cyto-fast fix/perm buffer set (BioLegend 420201), washed, and stained with anti-human/mouse Granzyme B Recombinant Antibody, clone QA16A02, PE/Dazzle™ 594 (BioLegend 372216) for 20 min at room temperature. Cells were washed twice with cell staining buffer and resuspended in 200 μL of cell staining buffer. Samples were acquired using a Cytek Northern Lights flow cytometry system. CD25⁺ of CD4⁺ or CD8⁺ T cells or Granzyme B⁺ of CD8⁺ T cells was used to measure PBMC activation after AMP inhibition by flow cytometry. Samples were analyzed using FlowJo version 10. Percent rescue (x-axis) versus inhibitor concentration (y-axis) was plotted using GraphPad Prism Version 6 and EC₅₀ values were generated using nonlinear regression analysis.

25 μL of reserved supernatant was transferred to a white 96-well plate (Corning cat no. 3917) and combined with 25 μL of a 1 μM adenosine 5′ triphosphate (ATP; MilliporeSigma cat no. A7699) solution. 50 μL of CellTiter-Glo reagent was added, and the plate incubated for 10 min at room temperature. Luminescence was measured using an EnSpire plate reader (PerkinElmer). Percent inhibition of adenosine production (x-axis) versus inhibitor concentration (y-axis) was plotted using GraphPad Prism Version 6 and EC₅₀ values were generated using nonlinear regression analysis.

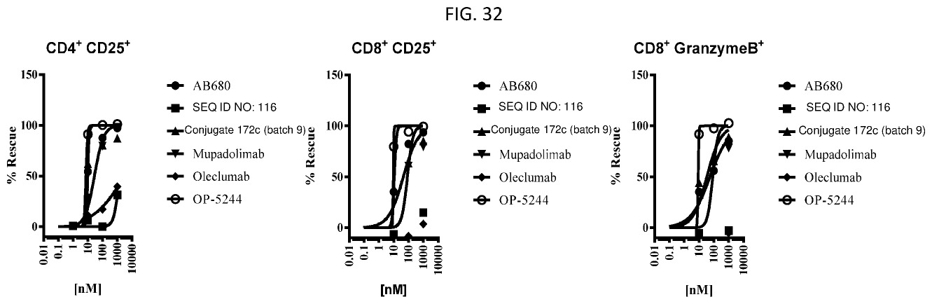

Conjugate 172c (batch 9) and five additional control compounds were tested to determine their ability to re-activate PBMCs that were inhibited by 30 μM AMP. SEQ ID NO: 116 is the Fc control for Conjugate 172c (batch 9) and had no activity in this assay (Table 21, FIG. 32). Conjugate 172c (batch 9) showed potent activity against 30 μM AMP, with EC₅₀ values ranging from 10 nM-44 nM. This was comparable to AB680, a potent small molecule CD73 inhibitor, had EC₅₀ values ranging from 10 nM to 54 nM. OP5244, another small molecule CD73 inhibitor, generated EC₅₀ values ranging from 7 nM-9 nM. Two anti-CD73 monoclonal antibodies, Mupadolimab and Oleclumab, were also included. Conjugate 172c (batch 9) was significantly more potent Oleclumab, which showed no activity in this assay. Conjugate 172c (batch 9) was more potent than Mupadolimab, which generated EC₅₀ values ranging from 28 nM to 84 nM (Table 21). Dose response curves are shown in FIG. 32.

TABLE 21
Activity (EC₅₀) of Conjugate 172c (batch 9) and
other test articles targeting CD73 in a human
PBMC activation assay by flow cytometry.

| Test | EC₅₀ [nM] (median, n = 3) | | | | |
|---|---|---|---|---|---|
| article | CD4⁺CD25⁺ | CD8⁺CD25⁺ | | CD8⁺Granzyme B⁺ | |
| Conjugate 172c | 10 | 15 | (13-44) | 34 | (10-66) |
| (batch 9) | | | | | |
| SEQ ID | >1,000 | >1,000 | | >1,000 | |
| NO: 116 | | | | | |
| AB680 | 10 | 10 | (10-11) | 47 | (5-54) |
| OP-5244 | 7 | 7 | (4-9) | 8 | (1-8) |
| Mupadolimab | 28 | 84 | (43-92) | 83 | (77-87) |

| | | | |
|---|---|---|---|
| Oleclumab | >1,000 | >1,000 | >1,000 |

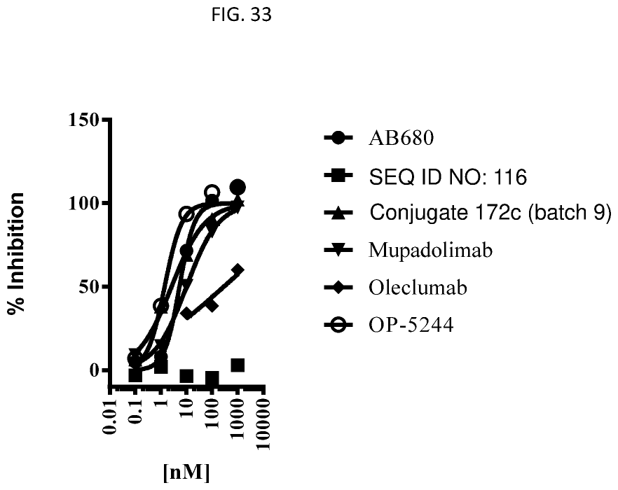

Similar EC₅₀ values were obtained when measuring AMP conversion by CellTiter-Glo. Conjugate 172c (batch 9), AB680, and OP5244 each showed potent activity with EC₅₀ values of 2 nM, 5 nM, and 1 nM, respectively. SEQ ID NO: 116 had no activity and Mupadolimab and Oleclumab generated EC₅₀ values of 10 nM and 288 nM, respectively (Table 22). Dose response curves are shown in FIG. 33.

TABLE 22
Activity of test articles targeting CD73 in a human PBMC activation
assay measuring AMP conversion by CellTiter-Glo.

| Test article | EC₅₀ [nM] |
|---|---|
| Conjugate 172c | 2 |
| (batch 9) | |
| SEQ ID | >1,000 |
| NO: 116 | |
| AB680 | 5 |
| OP-5244 | 1 |
| Mupadolimab | 10 |
| Oleclumab | 288 |

### CD73 Internalization Assay Using Conjugate 172c (Batch 9)

The ability of Conjugate 172c (batch 9) to induce receptor-mediated internalization of CD73 into the human breast adenocarcinoma cell line, MDA-MB-231 (ATCC CRM-HTB-26), was measured using the Fab-ZAP saporin conjugate assay kit (Advanced Targeting Systems cat no. IT-65-25) as previously described (Hay et al., 2016). Cells were plated into white tissue culture-treated 96 well plates (Corning cat no. 3917) at 2×10³ cells/well in 100 μL in RPMI (Gibco cat no. 11875093)+10% FBS (Gibco cat no. 16140071) and incubated overnight at 37° C. with 5% CO₂. Serial dilutions of test article (TA) from 0.06 nM-20 nM (2× final concentration) were pre-incubated with 100 nM Fab-ZAP reagent (2× final concentration) for 30 min at room temperature. Cells were washed once with 150 μL/well 1×PBS (Gibco cat no. 10010023), followed by the addition of 50 μL/well of media and 50 μL/well TA/Fab-ZAP mix. Cells were then incubated for 96 h at 37° C. with 5% CO₂ and viability was determined after 96 h via the CellTiter-Glo assay (Promega cat no. G7570) following the manufacturer's instructions. Cell viability RLU (y-axis) versus log₁₀ nM drug concentration (x-axis) was plotted using GraphPad Prism. The % CD73 internalization was calculated using the following formula: % CD73 internalization=[1−RLU (sample)/RLU (untreated cells)]×100%. The EC₅₀'s for each TA was calculated using the 4 PL non-linear regression analysis in GraphPad Prism software.

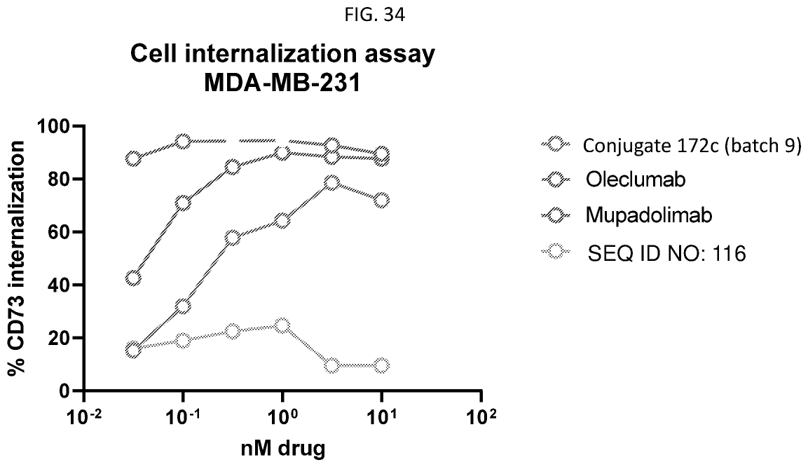

Conjugate 172c (batch 9) and the anti-CD73 monoclonal antibodies demonstrated potent sub-nanomolar CD73 internalization activity (Table 23, FIG. 34) resulting in >70% receptor internalization on the MDA-MB-231 cell line. The EC₅₀ for Mupadolimab (EC₅₀ 0.055 nM) is 2.5 fold lower than Conjugate 172c (batch 9) (EC₅₀ 0.141 nM), and the EC₅₀ for Oleclumab (EC₅₀>0.03 nM) is at least 5 fold lower than Conjugate 172c (batch 9). The human IgG Fc (SEQ ID NO: 116) control did not exhibit any internalization activity up to the maximum concentration tested (Table 23). The receptor internalization activity exhibited by Conjugate 172c (batch 9) may enhance anti-tumor activity versus tumor cells that express high levels of CD73.

TABLE 23
CD73 internalization EC₅₀'s of Conjugate 172c
(batch 9), Oleclumab, Mupadolimab, and hIgG Fc
(SEQ ID NO: 116) into MDA-MB-231 cancer cells.

| | | Cell internalization |
|---|---|---|
| Test article | DAR | EC₅₀ (nM) |
| Conjugate 172c | 7.9 | 0.141 |
| (batch 9) | | |
| Oleclumab | n/a | <0.03 |
| Mupadolimab | n/a | 0.055 |
| hIgG1 isotype control | n/a | >10 |
| (SEQ ID NO: 116) | | |
| AB680 | n/a | No activity |
| OP-5244 | n/a | No activity |

### Conjugate 172a 14-Day Dose Linearity Study

A mouse PK study was performed using female BALB/c (CRL cat no. 028BALB/C) mice 6-9 weeks of age. Mice were injected intravenously with either 1000 mpk, 300 mpk, 100 mpk, 30 mpk, 10 mpk, or 3 mpk of test article. Animals were housed under standard IACUC approved housing conditions.

At appropriate times animals were non-terminally bled (retro-orbital, cheek, or by tail vein) with blood collected in K₂EDTA tubes to prevent coagulation. Collected blood was centrifuged (2,000×g, for 10 min) and plasma withdrawn for analysis of test article concentrations over time. Plasma concentrations at each time point were measured using two separate ELISA methods: a human CD73 protein capture with Fc-detection, and a Fc-capture method with Fc-detection to compare and confirm that the intact molecule (containing at least one targeting moiety (Int) remained stable in vivo. For the CD73-capture, Nunc Maxisorp 96-well plates (ThermoFisher cat no. 12-565-136) were coated with 0.2 μg/100 μL/well human CD73 (Acro Biosciences cat. no CD3-H52H7) in carbonate buffer (MilliporeSigma cat no. C3041). Plates were incubated under static conditions at 4° C. overnight and washed 3× with 300 μL/well 1×PBS pH 7.4 (Fisher Scientific cat no. MT21040CM) supplemented with 0.05% Tween 20 (Fisher Scientific cat no. BP337-500) (PBST) and blocked with 200 μL/well 2% BSA (MilliporeSigma cat no. A5611) in TBS (Teknova T9867) supplemented with 0.05% Tween 20 (TBST) for 1 h at room temperature with shaking (400 rpm). After discarding the blocking solution, duplicate three-fold serial dilutions of the plasma samples were plated at 100 μL/well and incubated at room temperature for 2 h with shaking (sample diluent: 0.5% BSA in TBS 0.025% Tween 20+naïve mouse plasma at final concentration of 1:100). Five-fold dilutions of DFC standard curves ranging from 0.0064 ng/mL-500 ng/mL were run on each plate in duplicate. Following the 2 h incubation, plates were washed 3× with 300 μL/well PBST. Conjugate bound to CD73 on the plates was then probed with a HRP conjugated anti-human IgG Fc F(ab′)₂ (Jackson ImmunoResearch cat no. 709-036-098) diluted 1:2,000 in sample diluent (0.5% BSA in TBS 0.025% Tween 20) for 1 h at room temperature, with shaking and protected from light. Plates were then washed 3× with 300 μL/well PBST and developed with 100 μL/well TMB substrate (BD Biosciences cat no. 555214) for 7-8 min. The reaction was stopped with 100 μL/well 1N H₂SO₄. Absorbance was read at 450 nm with an EnSpire multimode plate reader (PerkinElmer). Concentration of DFC in plasma samples was interpolated using nonlinear regression analysis (Sigmoidal, 4 PL analysis) of the standard curves in GraphPad Prism (Version 8).

The Fc-capture ELISA was performed as described with the following modifications. Nunc Maxisorp 96-well plates were coated overnight at 4° C. under static conditions with 0.1 μg/100 μL/well of goat anti-human IgG (Fcγ fragment specific; Jackson ImmunoResearch cat no. 109-005-098) in carbonate buffer (MilliporeSigma cat no. C3041). Plates were washed 3× with 300 μL/well PBST and blocked with 200 μL/well in blocking solution (TBS+0.05% Tween 20+5% non-fat dry milk (Cell Signaling Technology cat no. 9999S) for 1 h at room temperature with shaking (400 rpm). After discarding the blocking solution, three-fold serial dilutions of the plasma samples were plated at 100 μL/well and incubated at room temperature for 2 h with shaking (sample diluent: 2.5% non-fat dry milk in TBS 0.025% Tween 20+naïve mouse plasma final concentration of 1:900). DFC standard curves ranging from 0.03 ng/mL to 55 ng/mL were run on each plate in duplicate. Following the 2 h incubation, plates were washed 3× with 300 μL/well PBST. Conjugate bound to Fc on the plates was then probed with 100 μL/well of HRP conjugated anti-human IgG Fc F(ab′)₂ (Jackson ImmunoResearch cat no. 709-036-098) diluted 1:2,000 in sample diluent (2.5% non-fat dry milk in TBS 0.025% Tween 20) for 1 h at room temp, with shaking and protected from light. Plates were then washed 3× with 300 μL/well PBST and developed with 100 μL/well TMB substrate reagent for 7-8 min. The reaction was stopped with 100 μL/well 1 N H₂SO₄ and the absorbance read at 450 nm with an EnSpire multimode plate reader (PerkinElmer). Concentration of DFC in plasma samples was interpolated using non-linear regression analysis (Sigmoidal, 4 PL analysis) of the standard curves in GraphPad Prism (Version 8).

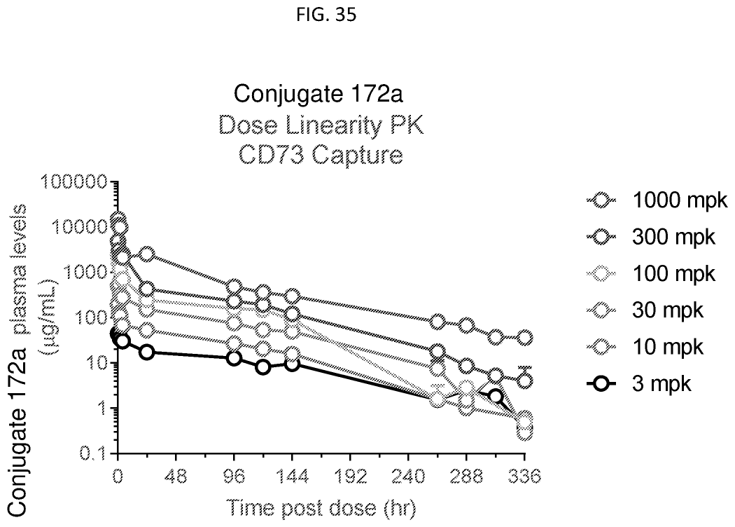

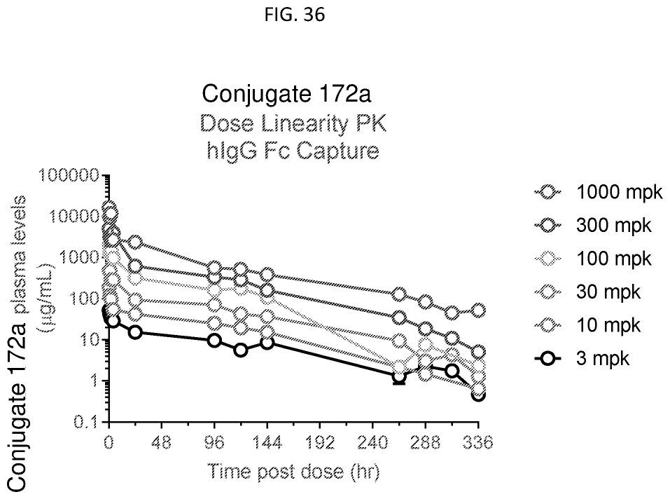

Each plasma concentration-time curve titrates according to dose, seen in both CD73 capture/Fc detection and Fc capture/Fc detection, FIG. 35 and FIG. 36, respectively. As the dose increased, the normalized dose dropped for both CD73 capture/Fc detection and Fc capture/Fc detection, per Table 24. This indicates some level of saturation of Conjugate 172a as the dose increases.

TABLE 24
Normalized dose of Conjugate 172a for both CD73 capture/Fc
detection and Fc capture/Fc detection; decrease of
normalized AUC as dose increases indicates saturation
of Conjugate 172a at higher doses.

| Dose | CD73 | Dose | hIgG | Dose |
|---|---|---|---|---|
| (mpk) | AUC | normalized AUC | Fc AUC | normalized AUC |
| 3 | 2952 | 984 | 2528 | 843 |
| 10 | 6735 | 674 | 5950 | 595 |
| 30 | 20297 | 677 | 16351 | 545 |
| 100 | 40995 | 410 | 49621 | 496 |
| 300 | 81252 | 271 | 117628 | 392 |
| 1000 | 231760 | 232 | 249821 | 250 |

### Conjugate 172a Rat PK Dose Linearity Study

A rat PK study was performed using male Sprague-Dawley rats at least 8 weeks of age. Rats were injected intravenously with either 150 mpk, 500 mpk, or 1000 mpk of test article. The 1000 mpk group was dosed twice, once at Day 0 and once at Day 8. Animals were housed under standard IACUC approved housing conditions. At appropriate times animals were non-terminally bled (retro-orbital, cheek, or by tail vein) with blood collected in K₂EDTA tubes to prevent coagulation. Collected blood was centrifuged and plasma withdrawn for analysis of test article concentrations overtime. Plasma concentrations at each time point were measured using two separate ELISA methods: a human CD73 protein capture with Fc-detection, and a Fc-capture method with Fc-detection to compare and confirm that the intact molecule (containing at least one targeting moiety (Int) remained stable in vivo. For the CD73-capture, Nunc Maxisorp 96-well plates (ThermoFisher cat no. 12-565-136) were coated with 0.2 μg/100 μL/well human CD73 (Acro Biosciences cat. no CD3-H52H7) in carbonate buffer (MilliporeSigma cat no. C3041). Plates were incubated under static conditions at 4° C. overnight and washed 3× with 300 μL/well 1×PBS pH 7.4 (Fisher Scientific cat no. MT21040CM) supplemented with 0.05% Tween 20 (Fisher Scientific cat no. BP337-500) (PBST) and blocked with 200 μL/well 2% BSA (MilliporeSigma cat no. A5611) in TBS (Teknova T9867) supplemented with 0.05% Tween 20 (TBST) for 1 h at room temperature with shaking (400 rpm). After discarding the blocking solution, duplicate three-fold serial dilutions of the plasma samples were plated at 100 μL/well and incubated at room temperature for 2 h with shaking (sample diluent: 0.5% BSA in TBS 0.025% Tween 20+naïve rat plasma at final concentration of 1:100). Five-fold dilutions of DFC standard curves ranging from 0.0064 ng/mL-500 ng/mL were run on each plate in duplicate. Following the 2 h incubation, plates were washed 3× with 300 μL/well PBST. Conjugate bound to CD73 on the plates was then probed with a HRP conjugated anti-human IgG Fc F(ab′)₂ (Jackson ImmunoResearch cat no. 709-036-098) diluted 1:2,000 in sample diluent (0.5% BSA in TBS 0.025% Tween 20) for 1 h at room temperature, with shaking and protected from light. Plates were then washed 3× with 300 μL/well PBST and developed with 100 μL/well TMB substrate (BD Biosciences cat no. 555214) for 7-8 min. The reaction was stopped with 100 μL/well 1N H₂SO₄. Absorbance was read at 450 nm with an EnSpire multimode plate reader (PerkinElmer). Concentration of DFC in plasma samples was interpolated using nonlinear regression analysis (Sigmoidal, 4 PL analysis) of the standard curves in GraphPad Prism (Version 8).

The Fc-capture ELISA was performed as described with the following modifications. Nunc Maxisorp 96-well plates were coated overnight at 4° C. under static conditions with 0.1 μg/100 μL/well of goat anti-human IgG (Fcγ fragment specific; Jackson ImmunoResearch cat no. 109-005-098) in carbonate buffer (MilliporeSigma cat no. C3041). Plates were washed 3× with 300 μL/well PBST and blocked with 200 μL/well in blocking solution (TBS+0.05% Tween 20+5% non-fat dry milk (Cell Signaling Technology cat no. 9999S) for 1 h at room temperature with shaking (400 rpm). After discarding the blocking solution, three-fold serial dilutions of the plasma samples were plated at 100 μL/well and incubated at room temperature for 2 h with shaking (sample diluent: 2.5% non-fat dry milk in TBS 0.025% Tween 20+naïve rat plasma final concentration of 1:900). DFC standard curves ranging from 0.03 ng/mL to 55 ng/mL were run on each plate in duplicate. Following the 2 h incubation, plates were washed 3× with 300 μL/well PBST. Conjugate bound to Fc on the plates was then probed with 100 μL/well of HRP conjugated anti-human IgG Fc F(ab′)₂ (Jackson ImmunoResearch cat no. 709-036-098) diluted 1:2,000 in sample diluent (2.5% non-fat dry milk in TBS 0.025% Tween 20) for 1 h at room temp, with shaking and protected from light. Plates were then washed 3× with 300 μL/well PBST and developed with 100 μL/well TMB substrate reagent for 7-8 min. The reaction was stopped with 100 μL/well 1 N H₂SO₄ and the absorbance read at 450 nm with an EnSpire multimode plate reader (PerkinElmer). Concentration of DFC in plasma samples was interpolated using non-linear regression analysis (Sigmoidal, 4 PL analysis) of the standard curves in GraphPad Prism (Version 8).

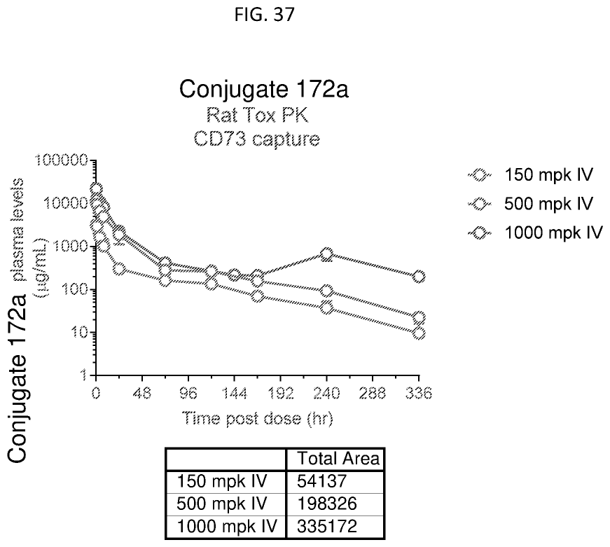

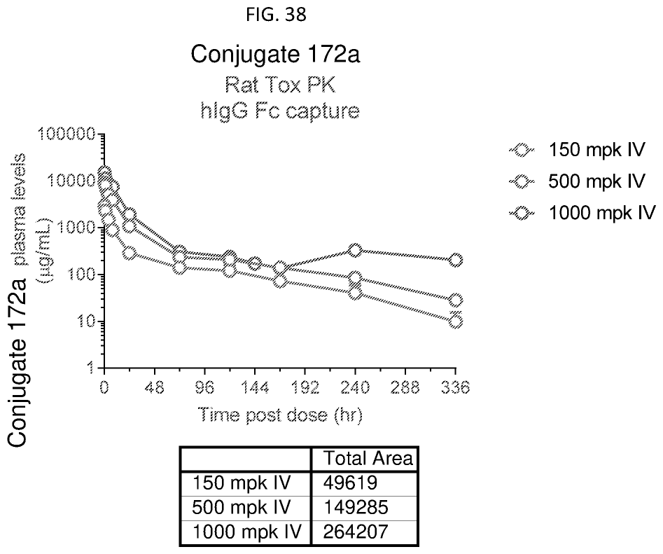

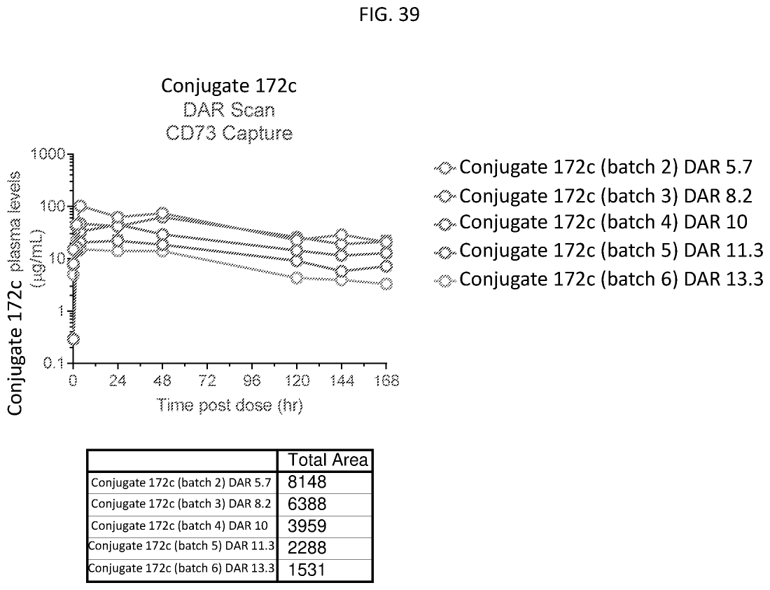

Each plasma concentration-time curve titrates according to dose, seen in both CD73 capture/Fc detection and Fc capture/Fc detection, FIG. 37 and FIG. 38, respectively. Per Table 25, the decrease of normalized AUC from 500 mpk to 1000 mpk indicates possible saturation of Conjugate 172a at higher doses.

TABLE 25
Normalized dose of Conjugate 172a for both CD73 capture/Fc
detection and Fc capture/Fc detection; decrease of normalized
AUC from 500 mpk to 1000 mpk indicates possible saturation
of Conjugate 172a at higher doses.

| Dose | CD73 | Dose | hIgG | Dose |
|---|---|---|---|---|
| (mpk) | AUC | normalized AUC | Fc AUC | normalized AUC |
| 150 | 48040 | 320 | 43166 | 288 |
| 500 | 183818 | 368 | 135718 | 271 |
| 1000 | 261166 | 261 | 221851 | 222 |

NOTE:
The AUCs included in Table 25 only include the first 168 hours of the PK study, as only the 1000 mpk group was dosed a second time on Day 8.

### Conjugate 172c DAR Scan Mouse PK Study

A mouse PK study was performed using female BALB/c (CRL cat no. 028BALB/C) mice 6-9 weeks of age. Mice were injected intraperitoneal (IP) with 10 mg/kg of test article (5 mL/kg dose volume). Animals were housed under standard IACUC approved housing conditions. At appropriate times animals were non-terminally bled (retro-orbital, cheek, or by tail vein) with blood collected in K₂EDTA tubes to prevent coagulation. Collected blood was centrifuged (2,000×g, for 10 min) and plasma withdrawn for analysis of test article concentrations over time. Plasma concentrations at each time point were measured using two separate ELISA methods: a human CD73 protein capture with Fc-detection, and a Fc-capture method with Fc-detection to compare and confirm that the intact molecule (containing at least one targeting moiety (Int) remained stable in vivo. For the CD73-capture, Nunc Maxisorp 96-well plates (ThermoFisher cat no. 12-565-136) were coated with 0.2 μg/100 μL/well human CD73 (Acro Biosciences cat. no CD3-H52H7) in carbonate buffer (MilliporeSigma cat no. C3041). Plates were incubated under static conditions at 4° C. overnight and washed 3× with 300 μL/well 1×PBS pH 7.4 (Fisher Scientific cat no. MT21040CM) supplemented with 0.05% Tween 20 (Fisher Scientific cat no. BP337-500) (PBST) and blocked with 200 μL/well 2% BSA (MilliporeSigma cat no. A5611) in TBS (Teknova T9867) supplemented with 0.05% Tween 20 (TBST) for 1 h at room temperature with shaking (400 rpm). After discarding the blocking solution, duplicate three-fold serial dilutions of the plasma samples were plated at 100 μL/well and incubated at room temperature for 2 h with shaking (sample diluent: 0.5% BSA in TBS 0.025% Tween 20+naïve mouse plasma at final concentration of 1:100). Five-fold dilutions of DFC standard curves ranging from 0.0064 ng/mL-500 ng/mL were run on each plate in duplicate. Following the 2 h incubation, plates were washed 3× with 300 μL/well PBST. Conjugate bound to CD73 on the plates was then probed with a HRP conjugated anti-human IgG Fc F(ab′)₂ (Jackson ImmunoResearch cat no. 709-036-098) diluted 1:2,000 in sample diluent (0.5% BSA in TBS 0.025% Tween 20) for 1 h at room temperature, with shaking and protected from light. Plates were then washed 3× with 300 μL/well PBST and developed with 100 μL/well TMB substrate (BD Biosciences cat no. 555214) for 7-8 min. The reaction was stopped with 100 μL/well 1 N H₂SO₄. Absorbance was read at 450 nm with an EnSpire multimode plate reader (PerkinElmer). Concentration of DFC in plasma samples was interpolated using nonlinear regression analysis (Sigmoidal, 4 PL analysis) of the standard curves in GraphPad Prism (Version 8).

The Fc-capture ELISA was performed as described with the following modifications. Nunc Maxisorp 96-well plates were coated overnight at 4° C. under static conditions with 0.1 μg/100 μL/well of goat anti-human IgG (Fcγ fragment specific; Jackson ImmunoResearch cat no. 109-005-098) in carbonate buffer (MilliporeSigma cat no. C3041). Plates were washed 3× with 300 μL/well PBST and blocked with 200 μL/well in blocking solution (TBS+0.05% Tween 20+5% non-fat dry milk (Cell Signaling Technology cat no. 9999S) for 1 h at room temperature with shaking (400 rpm). After discarding the blocking solution, three-fold serial dilutions of the plasma samples were plated at 100 μL/well and incubated at room temperature for 2 h with shaking (sample diluent: 2.5% non-fat dry milk in TBS 0.025% Tween 20+naïve mouse plasma final concentration of 1:900). Three-fold DFC standard curves ranging from 0.03 ng/mL to 55 ng/mL were run on each plate in duplicate. Following the 2 h incubation, plates were washed 3× with 300 μL/well PBST. Conjugate bound to Fc on the plates was then probed with 100 μL/well of HRP conjugated anti-human IgG Fc F(ab′)₂ (Jackson ImmunoResearch cat no. 709-036-098) diluted 1:2,000 in sample diluent (2.5% non-fat dry milk in TBS 0.025% Tween 20) for 1 h at room temp, with shaking and protected from light. Plates were then washed 3× with 300 μL/well PBST and developed with 100 μL/well TMB substrate reagent for 7-8 min. The reaction was stopped with 100 μL/well 1 N H₂SO₄ and the absorbance read at 450 nm with an EnSpire multimode plate reader (PerkinElmer). Concentration of DFC in plasma samples was interpolated using non-linear regression analysis (Sigmoidal, 4 PL analysis) of the standard curves in GraphPad Prism (Version 8).

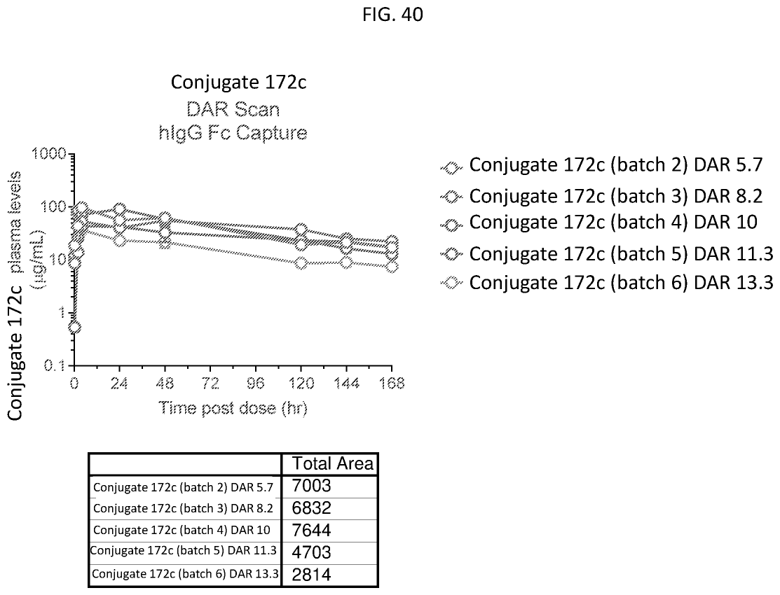

The 7-day mouse PK profiles for the DAR scan of Conjugate 172c (batches 2-6) showed that, for CD73 capture/Fc detection, drug exposure levels decrease as DAR increases per AUC. (FIG. 39). Additionally, for Fc capture/Fc detection, drug exposure levels decrease after a DAR of 10 per AUC. (FIG. 40).

### Conjugate 172a 14-Day Dose Route Study (IV, IP, SC)

A mouse PK study was performed using female BALB/c (CRL cat no. 028BALB/C) mice 6-9 weeks of age. Mice were injected intravenously, intraperitoneally, or subcutaneously with 3 mg/kg of test article. Animals were housed under standard IACUC approved housing conditions. At appropriate times animals were non-terminally bled (retro-orbital, cheek, or by tail vein) with blood collected in K₂EDTA tubes to prevent coagulation. Collected blood was centrifuged (2,000×g, for 10 min) and plasma withdrawn for analysis of test article concentrations overtime. Plasma concentrations at each time point were measured using two separate ELISA methods: a human CD73 protein capture with Fc-detection, and a Fc-capture method with Fc-detection to compare and confirm that the intact molecule (containing at least one targeting moiety (Int) remained stable in vivo. For the CD73-capture, Nunc Maxisorp 96-well plates (ThermoFisher cat no. 12-565-136) were coated with 0.2 μg/100 μL/well human CD73 (Acro Biosciences cat. no CD3-H52H7) in carbonate buffer (MilliporeSigma cat no. C3041). Plates were incubated under static conditions at 4° C. overnight and washed 3× with 300 μL/well 1×PBS pH 7.4 (Fisher Scientific cat no. MT21040CM) supplemented with 0.05% Tween 20 (Fisher Scientific cat no. BP337-500) (PBST) and blocked with 200 μL/well 2% BSA (MilliporeSigma cat no. A5611) in TBS (Teknova T9867) supplemented with 0.05% Tween 20 (TBST) for 1 h at room temperature with shaking (400 rpm). After discarding the blocking solution, duplicate three-fold serial dilutions of the plasma samples were plated at 100 μL/well and incubated at room temperature for 2 h with shaking (sample diluent: 0.5% BSA in TBS 0.025% Tween 20+naïve mouse plasma at final concentration of 1:100). Five-fold dilutions of DFC standard curves ranging from 0.0064 ng/mL-500 ng/mL were run on each plate in duplicate. Following the 2 h incubation, plates were washed 3× with 300 μL/well PBST. Conjugate bound to CD73 on the plates was then probed with a HRP conjugated anti-human IgG Fc F(ab′)₂ (Jackson ImmunoResearch cat no. 709-036-098) diluted 1:2,000 in sample diluent (0.5% BSA in TBS 0.025% Tween 20) for 1 h at room temperature, with shaking and protected from light. Plates were then washed 3× with 300 μL/well PBST and developed with 100 μL/well TMB substrate (BD Biosciences cat no. 555214) for 7-8 min. The reaction was stopped with 100 μL/well 1N H₂SO₄. Absorbance was read at 450 nm with an EnSpire multimode plate reader (PerkinElmer). Concentration of DFC in plasma samples was interpolated using nonlinear regression analysis (Sigmoidal, 4 PL analysis) of the standard curves in GraphPad Prism (Version 8).

The Fc-capture ELISA was performed as described with the following modifications. Nunc Maxisorp 96-well plates were coated overnight at 4° C. under static conditions with 0.1 μg/100 μL/well of goat anti-human IgG (Fcγ fragment specific; Jackson ImmunoResearch cat no. 109-005-098) in carbonate buffer (MilliporeSigma cat no. C3041). Plates were washed 3× with 300 μL/well PBST and blocked with 200 μL/well in blocking solution (TBS+0.05% Tween 20+5% non-fat dry milk (Cell Signaling Technology cat no. 9999S) for 1 h at room temperature with shaking (400 rpm). After discarding the blocking solution, three-fold serial dilutions of the plasma samples were plated at 100 μL/well and incubated at room temperature for 2 h with shaking (sample diluent: 2.5% non-fat dry milk in TBS 0.025% Tween 20+naïve mouse plasma final concentration of 1:900). DFC standard curves ranging from 0.03 ng/mL to 55 ng/mL were run on each plate in duplicate. Following the 2 h incubation, plates were washed 3× with 300 μL/well PBST. Conjugate bound to Fc on the plates was then probed with 100 μL/well of HRP conjugated anti-human IgG Fc F(ab′)₂ (Jackson ImmunoResearch cat no. 709-036-098) diluted 1:2,000 in sample diluent (2.5% non-fat dry milk in TBS 0.025% Tween 20) for 1 h at room temp, with shaking and protected from light. Plates were then washed 3× with 300 μL/well PBST and developed with 100 μL/well TMB substrate reagent for 7-8 min. The reaction was stopped with 100 μL/well 1 N H₂SO₄ and the absorbance read at 450 nm with an EnSpire multimode plate reader (PerkinElmer). Concentration of DFC in plasma samples was interpolated using non-linear regression analysis (Sigmoidal, 4 PL analysis) of the standard curves in GraphPad Prism (Version 8).

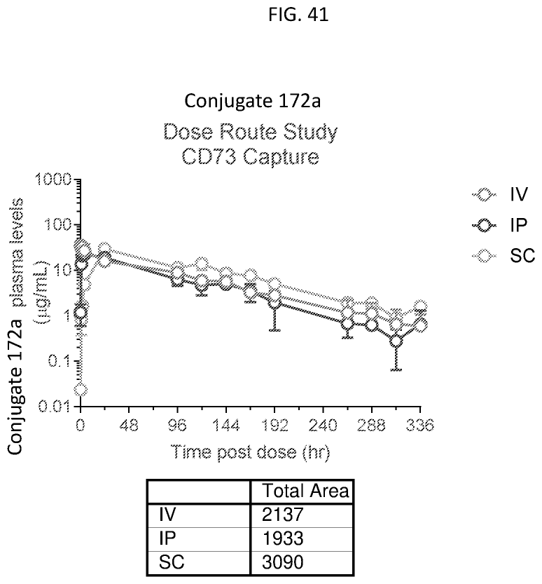

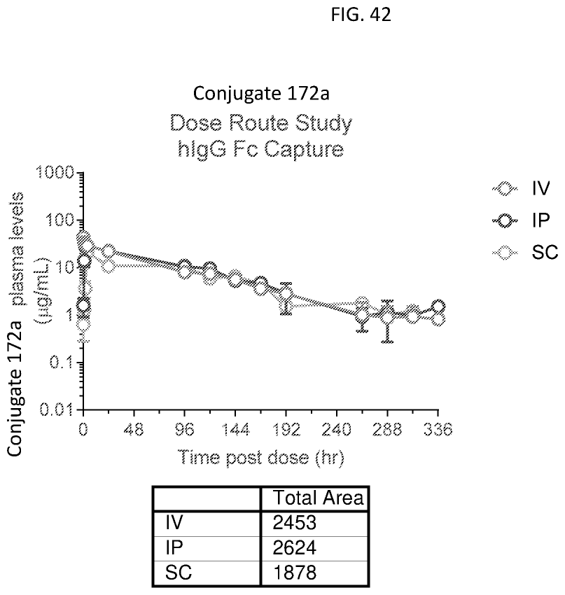

All three dose routes for Conjugate 172a showed similar drug exposure levels per AUC by both CD73 capture/Fc detection and Fc capture/Fc detection, as seen in FIG. 41 and FIG. 42, respectively. These data demonstrate that Conjugate 172a has high bioavailability for the IP and SC doses.

### Penetration of 3D Tumor Spheroids Using Conjugate 201

Penetration of drug-Fc conjugates (DFCs) into 3D tumor spheroids of mouse 4T1 (ATCC CRL-2539) cancer cells was determined. Briefly, 4T1 cancer cells were seeded at 1.5×10³ cells/well into ultra-low attachment 384 well plates (Corning cat no. 4516). Cells were centrifuged at 200×g to encourage spheroid formation and incubated at 37° C. with 5% CO₂ for 24 h. The next day, cells were centrifuged at 200×g and incubated at 37° C. with 5% CO₂ for an additional 24 h. Test articles were added at a final concentration of 100 nM and incubated at 37° C. with 5% CO₂ for 30 min. Cells were fixed, washed, blocked and stained with DyLight 488-labeled anti-human IgG Fc antibody (Novus Biological cat no. NBP2-60678G) for Oleclumab, unconjugated Fc (SEQ ID NO: 116) and irrelevant, non-CD73 targeting antibody control. Conjugate 201 was processed the same way except for staining with anti-human IgG Fc antibody as Conjugate 201 is a directly labeled green fluorescent molecule. Cells were scanned at 20× magnification on CQ1 confocal microscope (Yokogawa). Cell Pathfinder Software was used for segmentation and analysis. Statistical analysis was performed with unpaired t-test in GraphPad Prism software.

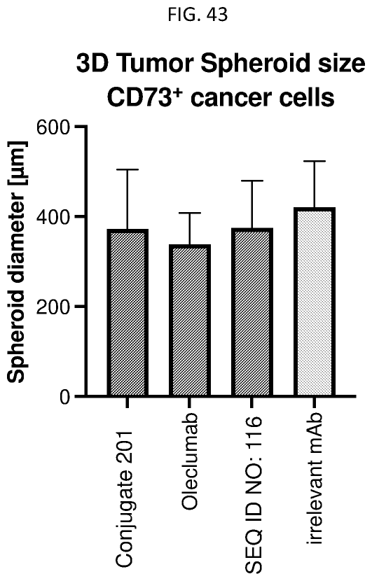

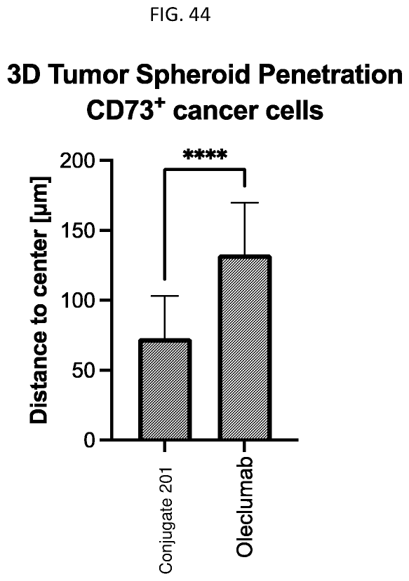

The 4T1 spheroids reached around 400 μM in mean diameter and were comparable in size between all test articles (FIG. 43). The mean distance of fluorescence signal to spheroid center was 73.0 μm for Conjugate 201 and 132.7 μm for Oleclumab (FIG. 44) demonstrating that the smaller DFC, Conjugate 201, penetrates deeper into the 3D tumor spheroid as compared to the larger, full-length mAb, Oleclumab.

Activity of Conjugate 172a in Combination with an α-PD-1 mAb in Mice

Tumor efficacy models were conducted in female BALB/c mice (Charles River laboratories; 18-22 g) injected with the breast tumor cell line EMT-6 (ATCC; CRL-2755). Briefly, EMT-6 cells propagated and prepared by standard methods were resuspended in PBS and injected subcutaneously (in a 100 μL volume) into the shaved right flank of mice. Five days after cell injection, and prior to measurable tumor formation, treatment was initiated via IP injection (10 mL/kg) of test articles or controls. Controls consisted of a vehicle only group (PBS), a group dosed daily with AB680 at 30 mg/kg (MedchemExpress, cat. HY-125286), and a final group dosed on Day 0, 2, 4, 7, 9, and 12 with an anti-PD-1 mAb (Bio-X-Cell, RMP1-14). The test article was Conjugate 172a dosed at 30 mg/kg twice weekly, for 3 weeks. The final group consisted of mice dosed with both the anti-PD-1 mAb and Conjugate 172a at 30 mg/kg using the respective dosing schedules for each.

Each group consisted of 15 animals and tumors were measured 3× weekly with calipers on Mon/Wed/Fri. Tumor volumes were calculated based on the following formula: [(width×width×length)/2]. All tumors were monitored until the maximum allowed volume was reached (=>2,000 mm³).

Although occasionally an animal would be removed from study if the tumor grew internally causing undo stress to the animal, or the inability to measure the tumor accurately. Data was analyzed and graphed with Graphpad Prism 6.00. Where conducted, statistical evaluation used the Mann-Whitney variation of the t-test.

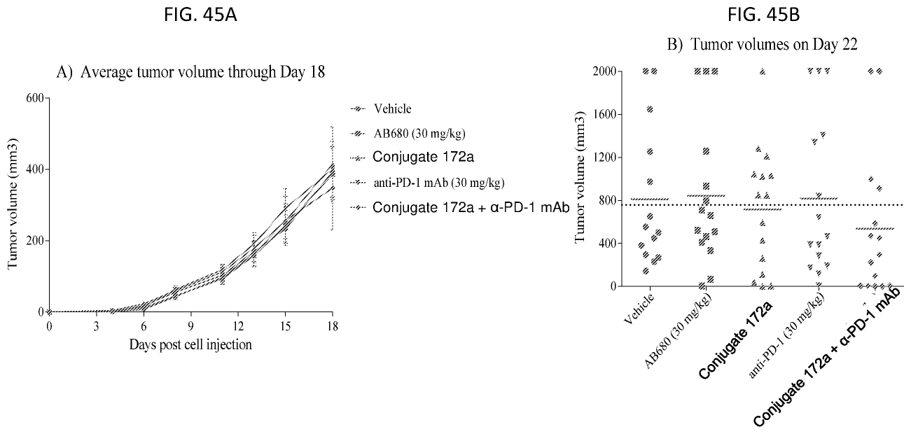

Through Day 18 (Day 13 post-treatment initiation) no statistical difference in tumor volumes was evident in any group (FIG. 45A). However, by Day 22 the combined treatment of Conjugate 172a with an anti-PD-1 mAb demonstrated a reduction in tumor volume of close to 30% relative to vehicle (FIG. 45B). The ability of CD73 targeting immunotherapies to enhance the activity of anti-PD-1 treatment is well documented in the literature (Allard et. al., 2013; Roh et. al., 2020). This observation indicates that Drug Fc-conjugates are capable of the same synergistic effect in combination with anti-PD-1 therapies, and has the potential to enhance the activity of other immune checkpoint inhibitors as well.

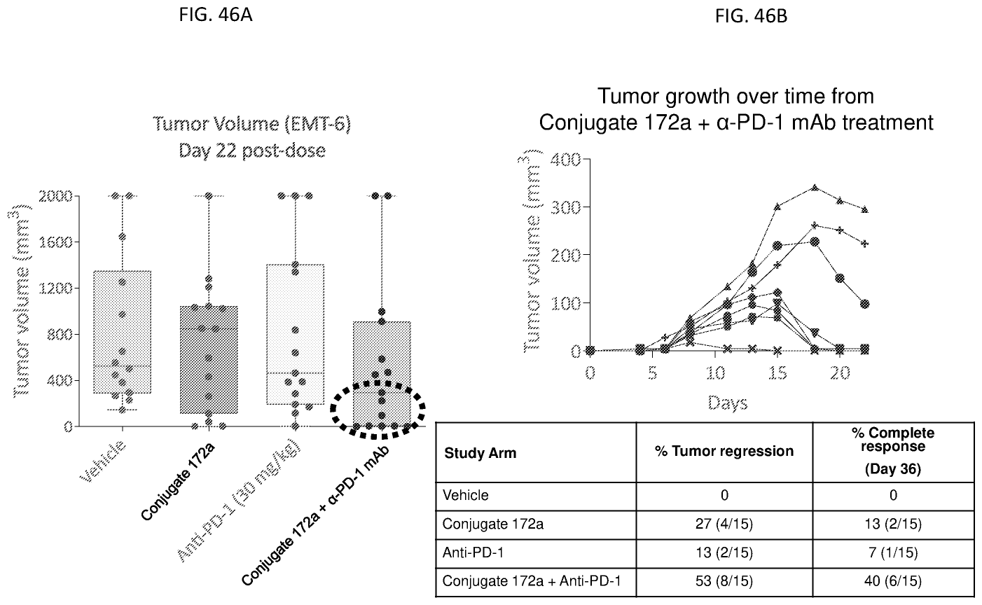

Even more striking than the reduction in tumor volume with combined treatment of Conjugate 172a with an anti-PD-1 mAb is the observation that five mice had fully regressed tumors by Day 22, compared to none in the vehicle only treatment group (FIG. 45B and FIG. 46A). By Day 36, an additional mouse in the combination treatment group reached full regression as well, resulting in 6 of 15 mice with un-measurable tumors (Table 26 and FIG. 46B). While 33.3% of mice receiving combination therapy demonstrated resolved tumors only 13.3 and 6.7% of mice treated with Conjugate 172a or anti-PD-1 monotherapy, respectively, reached full regression. In addition to the reduction in tumor volume, this is a second metric indicating that the anti-CD73 inhibitor effect of Conjugate 172a is complementary with established anti-PD-1 therapies.

TABLE 26
Percent tumor reduction (Day 22) and complete
regression (Day 36) of treatment groups.

| | % Tumor | % Complete |
|---|---|---|
| | reduction | regression |
| Treatment | (Day 22) | (Day 36) |
| AB680 | 0 | 6.7 |
| Conjugate 172a | 5.5 | 13.3 |
| α-PD-1 mAb | 0 | 6.7 |
| Conjugate 172a/α-PD-1 | 29.4 | 40.0 |

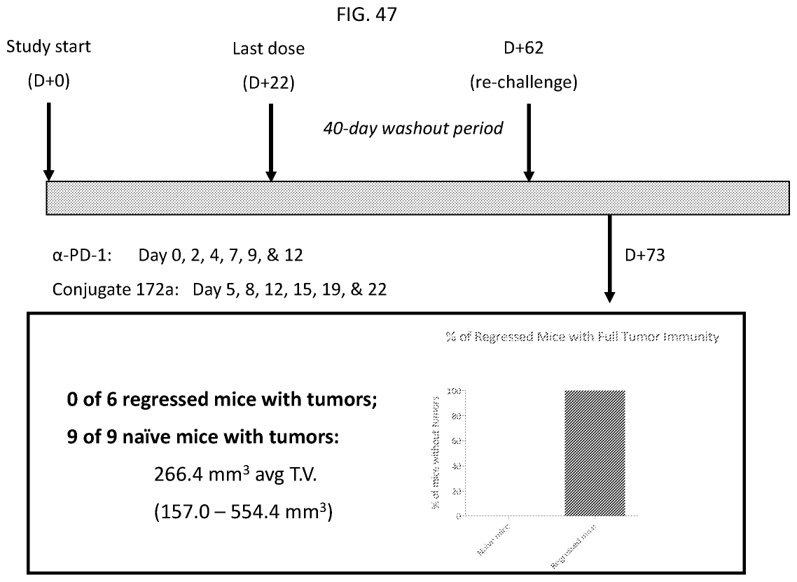

A final aspect of this study was to determine if fully regressed animals treated with the Conjugate 172a/anti-PD-1 combination also acquired immunity to the EMT-6 cancer cell line. To asses this, we allowed 40 days to pass since the last dose of any therapeutic (compound washout period) and then re-challenged regressed animals, along with nine naïve mice with EMT-6. The overall timeline for the study is detailed in FIG. 47. Eleven days after the EMT-6 re-challenge, all naïve mice demonstrated robust tumor growth as expected with an average tumor volume of 266.4 mm³. In contrast, none of the mice which had fully resolved their original tumors showed any growth (FIG. 47, box). And no tumor growth was detectable out to Day 83 in regressed animals, when the study was terminated.

Overall this study demonstrated the ability of Conjugate 172a, previously demonstrated to be a potent inhibitor of CD73, to enhance the activity of anti-PD-1 immune therapeutics, which are currently in use to treat various cancers. The enhanced activity of the Conjugate 172a/anti-PD-1 mAb combination was evident by both tumor volume reduction and percent full tumor regression. Importantly, full regression corresponded to complete immunity to the original cancer cell line. Collectively this data suggests Conjugate 172a in conjunction with anti-PD-1 treatment may be of clinical benefit to cancer patients.

### Activity of Conjugate 172a Versus Vehicle Against a Breast Cancer Cell Line (EMT-6) in a Syngeneic Mouse Model

A tumor efficacy models was conducted in female BALB/c mice (Charles River laboratories; 18-22 g) injected with the breast tumor cell line EMT-6 (ATCC; CRL-2755). Briefly, EMT-6 cells propagated and prepared by standard methods were resuspended in PBS and injected subcutaneously (in a 100 μL volume) into the shaved right flank of mice. Five days after cell injection, and prior to measurable tumor formation, treatment was initiated via IP injection (10 mL/kg) of Conjugate 172a (30 mg/kg, twice weekly for 3 weeks) or a vehicle only control.

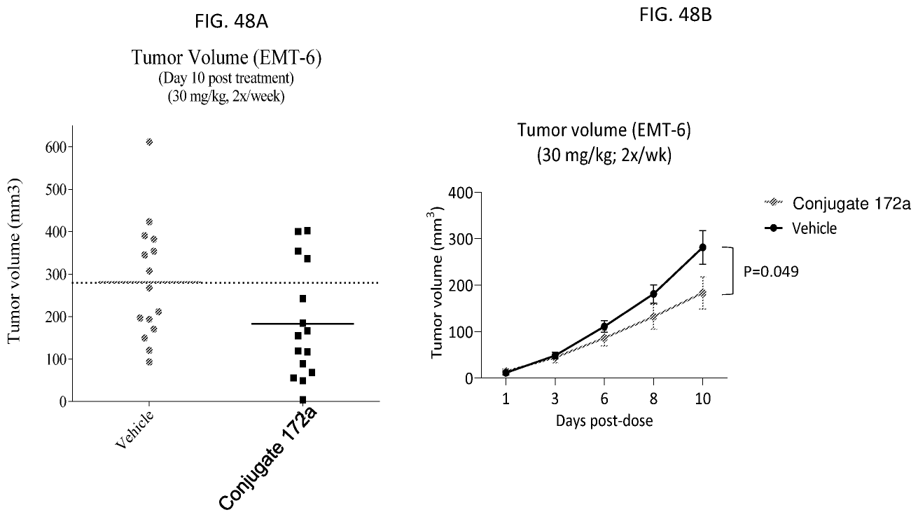

Relative to the vehicle control, the Conjugate 172a treatment group achieved a 35% reduction in tumor volume 10 days after treatment initiation (FIG. 48A and FIG. 48B). Importantly, this difference was statistically significant (P=0.049). Collectively this study indicates CD73 inhibition by Conjugate 172a is able to significantly reduce tumor growth against an important breast cancer line in a syngeneic mouse model.

### PBMC Activation Assay to Assess the Impact of Linker Length on In Vitro Potency

Human PBMCs (StemCell Technologies cat no. 70025) prepared in ImmunoCult™—XF T cell Expansion medium (StemCell Technologies cat no. 10981) were seeded at 1×10⁵ cells per well (100 μL of 1×10⁶ cells/mL) in the presence or absence of T cell activator anti-CD3/CD28/CD2 (StemCell Technologies cat no. 10970) in 96 well round bottom plates (Corning cat no. 3788). Ten-fold serial dilutions of each test article was prepared in medium (ranging from 12,000 nM-1.2 nM), resulting in final concentrations ranging from 3,000 nM-0.3 nM. 50 μL of test article was added to the cells, followed by 50 μL of adenosine 5′ monophosphate (AMP; MilliporeSigma cat no. A1752) for a final concentration of 30 μM. Plates were incubated at 37° C. with 5% CO₂ for 72 h. After 72 h the cells were centrifuged for 5 min at 300×g at 4° C.

Cells were washed twice with cell staining buffer (BioLegend cat no. 420201). Cells were incubated with Fc block (Unlabeled, Clone: 3070; BD Biosciences cat no. 5642220) for 10 min at 4° C. Cells were stained with anti-human CD25 antibody, clone 2A3, PE (StemCell Technologies cat no. 60153PE) and anti-human CD8 Antibody, clone SK1, PerCP (BioLegend 344708) for 20 min at 4° C. protected from light. Cells were washed twice with cell staining buffer and resuspended in 200 μL of cell staining buffer. Samples were acquired using a Cytek Northern Lights flow cytometry system. CD25⁺ of CD8⁺ T cells was used to measure PBMC activation after AMP inhibition by flow cytometry. Samples were analyzed using FlowJo version 10. Percent rescue (x-axis) versus inhibitor concentration (y-axis) was plotted using GraphPad Prism Version 6 and EC₅₀ values were generated using nonlinear regression analysis.

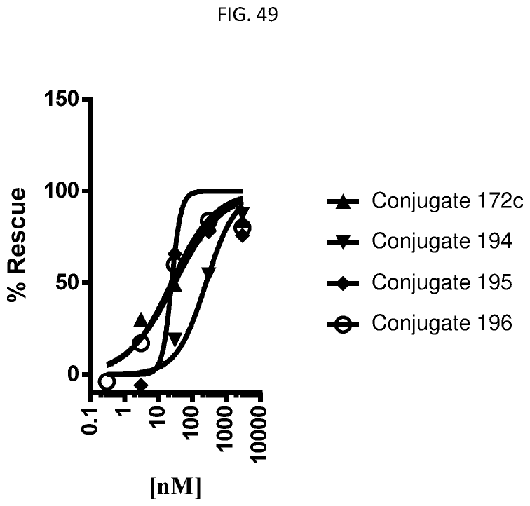

To assess the impact of linker length on in vitro potency, four compounds with the same small molecule targeting moiety (Int) but varying PEG linker lengths were tested. Linker lengths ranged from PEG4-PEG16. Conjugate 194 generated an EC₅₀ value of 253 nM. While Conjugate 194 does have the shortest linker, PEG4, it is difficult to conclude the ˜10× loss is potency is entirely due to linker length because Conjugate 194 has a lower DAR (6.2) than the other three compounds (DAR range 7.4-7.5). Conjugate 172c, Conjugate 195, and Conjugate 196 generated EC₅₀ values of 30 nM, 24 nM and 24 nM respectively, showing increasing linker length does not increase potency between the PEG8 and PEG16 (Table 27). The dose response curves are shown in FIG. 49.

TABLE 27
Activity of conjugates targeting CD73 in a PBMC activation assay
using CD25⁺ of CD8⁺ T cells as a read out.

| | Linker | | EC₅₀ |
|---|---|---|---|
| Test article | Length | DAR | [nM] |
| Conjugate 194 | PEG4 | 6.2 | 253 |
| Conjugate 172c | PEG8 | 7.5 | 30 |
| Conjugate 195 | PEG12 | 7.4 | 24 |
| Conjugate 196 | PEG16 | 7.4 | 24 |

### CD73 Enzyme Inhibition Assays Using Conjugate 172c

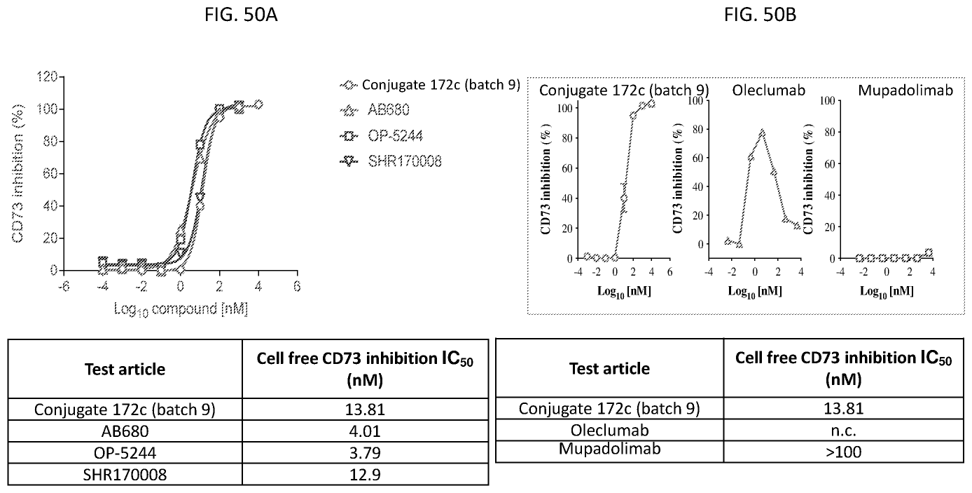

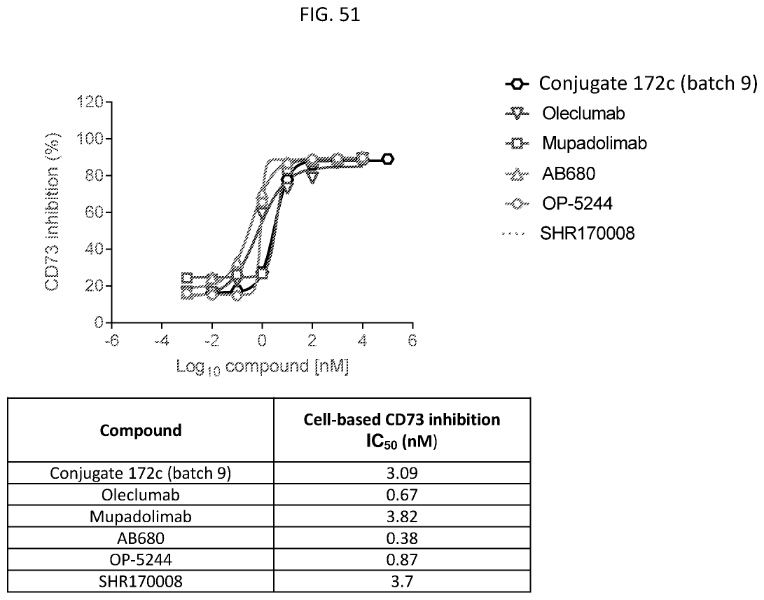

The cell-free CD73 enzyme inhibition assay was performed in a 96-well plate format using the CD73 Inhibitor Screening Assay Kit in accordance with the manufacturer's instructions (BPS Bioscience cat no. 72055). Briefly, test inhibitors and the positive control (AB680) were incubated in assay buffer (6 μL/well) in the presence of AMP (10 μL/well, final 100 μM) and recombinant human CD73 enzyme (0.1 ng/μL, 20 μL/well) for 25 min at 37° C. Colorimetric detection reagent was then added (100 μL/well) and the free phosphate from the CD73 reaction was measured by reading the absorbance at 630 nm using the EnSpire multimode plate reader (PerkinElmer). Wells without test inhibitor (CD73 positive control) were included on each plate. Blank wells without test inhibitor and CD73 were subtracted from all wells. Percent CD73 inhibition (y-axis) versus log₁₀ inhibitor concentration (x-axis) was plotted using GraphPad Prism Version 8 and IC₅₀'s calculated using nonlinear regression analysis (Sigmoidal, 4 PL). Conjugate 172c (batch 9) had single digit nM potency in the cell-free CD73 inhibition assay with an IC₅₀ of 13.8 nM (FIG. 50A), which was within approximately 1.1-3.6-fold of small molecule benchmark inhibitors including AB680 (IC₅₀ 4.0 nM), SHR170008 (IC₅₀ 12.9 nM) and OP-5244 (IC₅₀ 3.8 nM) (FIG. 50A and Table 28). In addition, CD73-targeting monoclonal antibodies Oleclumab and Mupadolimab were also tested, and both antibodies failed to inhibit soluble CD73 (FIG. 50B and Table 28).

TABLE 28
Activity of Conjugate 172c (batch 9) and other test
articles in a cell free CD73 inhibition assay.

| | Cell free CD73 |
|---|---|
| Test article | inhibition IC₅₀ (nM) |
| Conjugate 172c | 13.81 |
| (batch 9) | |
| AB680 | 4.01 |
| OP-5244 | 3.79 |
| SHR170008 | 12.9 |
| Oleclumab | n.c. |
| Mupadolimab | >100 |

The cell-based CD73 enzyme inhibition assay was performed essentially as described above for the cell-free assay, with the following modifications. MDA-MB-231 human breast adenocarcinoma cells (ATCC CRM-HTB-26) were seeded at 2×10⁴ cells/well/100 μL into 96-well tissue culture-treated plates (Corning Costar cat no. 3596) and incubated overnight at 37° C., 5% CO₂. Cells were incubated with test inhibitors (50 μL/well) and AMP (25 μL/well, final 400 μM) for 3 h at 37° C., 5% CO₂ to allow for interaction with CD73 expressed on the MDA-MB-231 cell surface. The 96-well plate was then centrifuged at 125×g for 5 min at room temperature and ATP (25 μL/well, 100 μM final) was combined with supernatant (25 μL/well) in a white opaque 96-well plate (Corning Costar cat no. 3912). The CD73 activity in the supernatant was quantified using the CellTiter-Glo kit (Promega cat no. G7571). Percent CD73 inhibition (y-axis) versus log₁₀ inhibitor concentration (x-axis) were plotted using GraphPad Prism Version 8 and IC₅₀'s calculated using nonlinear regression analysis (Sigmoidal, 4 PL). Conjugate 172c (batch 9) had single digit nanomolar potency in the MDA-MB231 cell-based CD73 inhibition assay with an IC₅₀ of 3.1 nM (FIG. 51), which was within approximately 3.4-7.8-fold of anti-CD73 monoclonal antibody Oleclumab (IC₅₀ 0.7 nM), and small molecule AB680 (IC₅₀ 0.4 nM) and OP-5244 (IC₅₀ 0.9 nM). Conjugate 172c (batch 9) demonstrated a modest ˜1.2-fold enhancement in potency compared to anti-CD73 monoclonal antibody Mupadolimab (IC₅₀ 3.8 nM) and small molecule SHR170008 (IC₅₀ 3.7 nM) (FIG. 51 and Table 29). Conjugate 172c (batch 9) (IC₅₀ 13.8 nM) and SHR170008 (IC₅₀ 12.9 nM) also showed similar potencies in the cell-free CD73 enzyme inhibition assay (FIG. 50A), while AB680 (IC₅₀4.0 nM) and OP-5244 (IC₅₀ 3.8 nM) showed increased potency. In the cell-based CD73 enzyme inhibition assay (FIG. 51 and Table 29) Conjugate 172c (batch 9) (IC₅₀ 3.1 nM), Mupadolimab (IC₅₀ 3.8 nM) and SHR170008 (IC₅₀ 3.7 nM) all showed similar and good potencies. Oleclumab (IC₅₀ 0.7 nM), AB680 (IC₅₀ 0.4 nM) and OP-5244 (IC₅₀ 0.9 nM) all showed increased potency when compared to Conjugate 172c (batch 9).

TABLE 29
Activity of Conjugate 172c (batch 9) and other test articles
in cell-based CD73 inhbition assay using MDA-MB-231 cells.

| | Cell-based CD73 |
|---|---|
| Test article | inhibition IC₅₀ (nM) |
| Conjugate 172c | 3.09 |
| (batch 9) | |
| Oleclumab | 0.67 |
| Mupadolimab | 3.82 |
| AB680 | 0.38 |
| OP-5244 | 0.87 |
| SHR170008 | 3.7 |

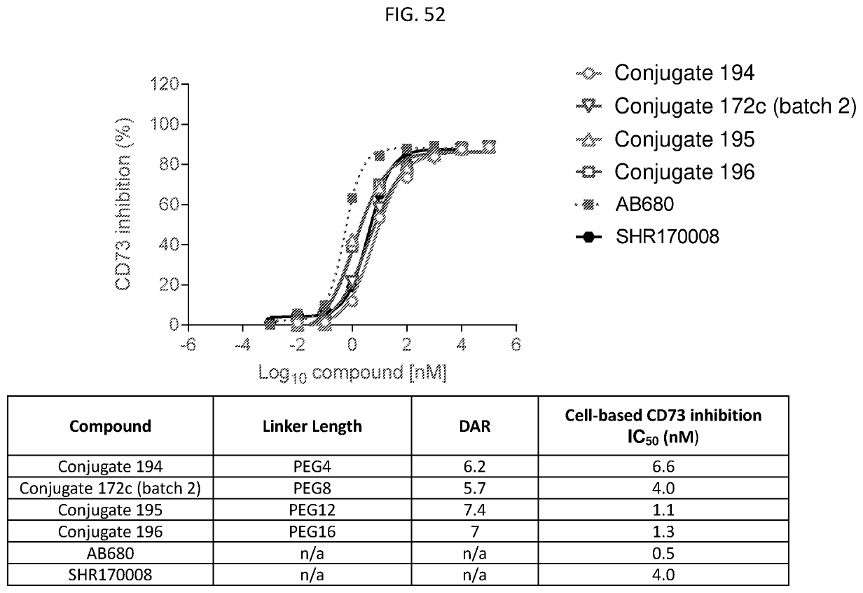

An additional assay was performed to test the effect of linker length on IC₅₀ potency using the cell-based CD73 enzyme inhibition assay. Conjugate 194, Conjugate 195, and Conjugate 196 have the same small molecule targeting moiety (Int) as Conjugate 172c (batch 9) but with varying linker lengths. Conjugate 194 (DAR 6.2, IC₅₀ 6.6 nM) has a linker length of PEG4, Conjugate 172c (batch 2) (DAR 5.7, IC₅₀ 4.0 nM) has a linker length of PEG8, Conjugate 195 (DAR 7.4, IC₅₀ 1.1 nM) has a linker length of PEG12, and Conjugate 196 (DAR 7.0, IC₅₀ 1.3 nM) has a linker length of PEG16. Linker length does appear to have an effect on IC₅₀ in the cell based CD73 enzyme inhibition assay (FIG. 52 and Table 30), with longer linker length trending towards enhanced potency.

TABLE 30
Activity of conjugates of various linker lengths
in cell-based CD73 inhibition assay.

| | Linker | | Cell-based CD73 |
|---|---|---|---|
| Test Article | Length | DAR | inhibition IC₅₀ (nM) |
| Conjugate 194 | PEG4 | 6.2 | 6.6 |
| Conjugate 172c | PEG8 | 5.7 | 4.0 |
| (batch 2) | | | |
| Conjugate 195 | PEG12 | 7.4 | 1.1 |
| Conjugate 196 | PEG16 | 7 | 1.3 |
| AB680 | n/a | n/a | 0.5 |
| SHR170008 | n/a | n/a | 4.0 |

Dose Response of Conjugate 172c (Batch 9) Alone and in Combination with an α-PD-1 mAb, in a Mouse Colon Tumor Model

Tumor efficacy models were conducted in female BALB/c mice (Charles River laboratories; 18-22 g) injected with the colon tumor cell line MC-38 (Kerafast ENH204-FP). Briefly, MC-38 cells propagated and prepared by standard methods were resuspended in PBS, mixed 1:1 with Matrigel (Fisher Scientific, CB-40234C) and injected subcutaneously (200 μL volume) into the shaved right flank of mice. Five days after cell injection, and prior to measurable tumor formation, treatment was initiated via IP injection (10 mL/kg) of test articles/controls.

Groups consisted of the following 1) Vehicle only (20 mM histidine, pH 5.5); 2) α-PD-1 mAb (Bio-X-Cell, RMP1-14) at 30 mg/kg; 3) Conjugate 172c (batch 9) at 2 mg/kg; 4) Conjugate 172c (batch 9) at 10 mg/kg; 5) Conjugate 172c (batch 9) at 50 mg/kg; and 6) Conjugate 172c (batch 9) (10 mg/kg)+α-PD-1 mAb (30 mg/kg). Groups 1, 3, 4, and 5 were dosed twice weekly for two weeks. Group 2 was dosed on Day 0, 2, 4, 7, 9, and 12. For the combination arm (group 6) each test article was dosed using its above dosing schedule.

Each group consisted of 12 mice except the Conjugate 172c/α-PD-1 group which had 10 animals. Tumors were measured 3× weekly with calipers on Mon/Wed/Fri. Tumor volumes were calculated based on the following formula: (width×width×length)/2. All tumors were monitored until the maximum allowed volume was reached (=>2,000 mm³), although occasionally an animal would be removed from study if the tumor grew internally causing undo stress to the animal, or the inability to measure the tumor accurately. Data was analyzed and graphed with Graphpad Prism 6.00. Where conducted, statistical evaluation used the Mann-Whitney variation of the t-test.

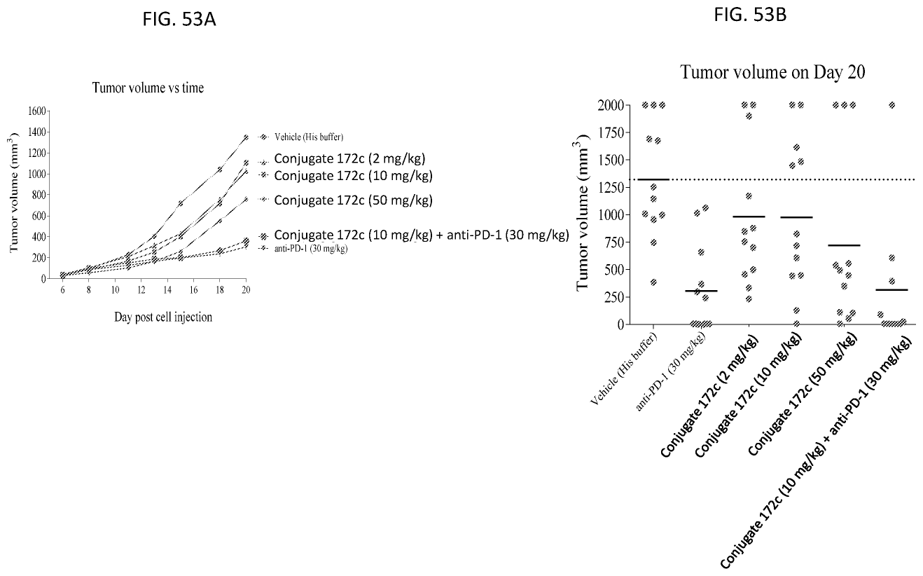

This study demonstrated a marked reduction in tumor volume at all Conjugate 172c concentrations (2, 10, and 50 mg/kg) when administered as a monotherapy (FIG. 53A). A clear dose response was also evident up to Day 13 although the 2 and 10 mg/kg dose groups began to overlap at later time points. The reduction in tumor volume seen with all Conjugate 172c groups also persisted after animals came off therapy (last dose of Conjugate 172c occurred on Day 16).

Tumor growth inhibition (TGI) relative to vehicle was determined for all groups from Day 13 through 20 (Table 31). For Conjugate 172c monotherapy groups the peak TGI occurred on Day 15 with a greater than 40% TGI even at the lowest dose of 2 mg/kg, although this was not statistically significant due to the limited n used in the study. However, when Conjugate 172c was dosed at 50 mg/kg the TGI increased to nearly 64%, which was statistically significant, as where all the TGIs from Days 13-20 for this dose group.

TABLE 31
Tumor growth inhibition by Conjugate 172c (batch
9) alone and in combination with α-PD-1 relative
to vehicle in a mouse colon tumor model.

| Test article | % TGI | | | |
|---|---|---|---|---|
| (mg/kg) | Day 13 | Day 15 | Day 18 | Day 20 |
| Conjugate 172c (2) | 22 | 40.3 | 21 | 25.8 |
| Conjugate 172c (10) | 36.8 | 40.7 | 24.8 | 26.1 |
| Conjugate 172c (50) | 59** | 63.7** | 42* | 45.4* |
| α-PD-1 (30) | 59.3** | 72.5** | 75.2*** | 77*** |
| Conjugate 172c (10) + | 53.1** | 72*** | 71.9** | 76.4*** |
| α-PD-1 (30) | | | | |

Relative to vehicle.
*P < 0.05;
**P < 0.01;
***P < 0.001

Based on the potency of the α-PD-1 mAb alone, it was not possible to see a benefit in combination with Conjugate 172c based on tumor volume alone (Table 31). However, an apparent benefit of Conjugate 172c when dosed in combination with α-PD-1 therapy is seen when analyzing individual animals. In the α-PD-1 monotherapy arm 50% of mice have tumors of less than 100 mm³. In combination with Conjugate 172c this number increases to 70%. This difference is not obvious from TGI values alone because of a single mouse in the combination group with a very larger tumor (2,000 mm³) (FIG. 53B).

Collectively this data demonstrates the potency of Conjugate 172c as a monotherapy against an aggressive tumor cell line even at doses as low as 2 mg/kg. At 50 mg/kg, the activity of Conjugate 172c is not statistically different than the α-PD-1 mAb comparator, a clinically validated target for immunotherapy. Lastly, this study indicates that Conjugate 172c dosed in combination with α-PD-1 mAb has a beneficial effect, supporting previous preclinical studies run with this pairing.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. All publications, patents, and patent applications mentioned in the above specification are hereby incorporated by reference to the same extent as if each individual publication, patent, or patent application was specifically and individually indicated to be incorporated by reference in its entirety.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

1. A conjugate, or a pharmaceutically acceptable salt thereof, described by formula (D-I) or (M-I): 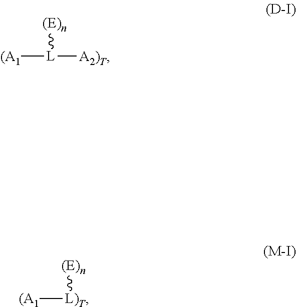
wherein each of A¹ and A², independently, has the structure of formula (A): 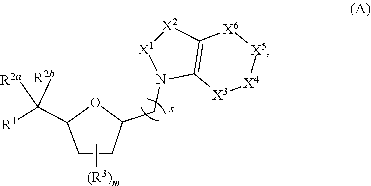
m is 0, 1, 2, 3, 4, 5, or 6;
s is 0 or 1;
each of X¹, X², X³, X⁴, X⁵, and X⁶ is, independently, N, CR⁴, or C—Y—R⁵, wherein at least one of X¹, X², X³, X⁴, X⁵, and X⁶ is C—Y—R⁵ and R⁵ is a bond to L;
R¹ is 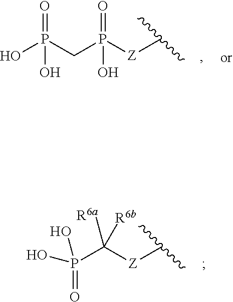
Z is O, S, or sulfonyl;
each of R^{2a} and R^{2b} is, independently, H, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl;
each R³ is, independently, OH, SH, halogen, optionally substituted amino, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl;
R⁴ is H, halogen, OH, SH, optionally substituted amino, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl;
each of R^{6a} and R^{6b} is, independently, H, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl;
Y is a first linker;
L is a second linker;
n is 1 or 2;
each E comprises an Fc domain monomer;
T is an integer from 1 to 20; and
the squiggly line indicates that L is covalently attached to E.

2. The conjugate of claim 1, or a pharmaceutically acceptable salt thereof, wherein the conjugate is described by formula (D-I):

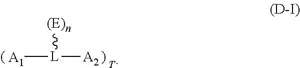

3. The conjugate of claim 1, or a pharmaceutically acceptable salt thereof, wherein the conjugate is described by formula (M-I):

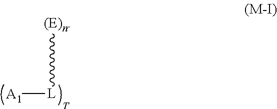

4. The conjugate of any one of claims 1-3, or a pharmaceutically acceptable salt thereof, wherein A¹ and A² have the structure of formula (A-I):

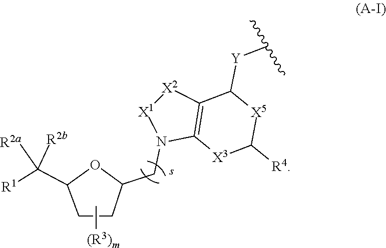

5. The conjugate of claim 4, or a pharmaceutically acceptable salt thereof, wherein A¹ and A² each have the structure of formula (A-Ia):

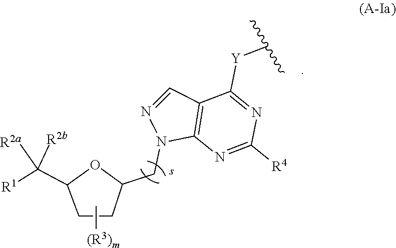

6. The conjugate of claim 5, or a pharmaceutically acceptable salt thereof, wherein A¹ and A² each have the structure of formula (A-Ib):

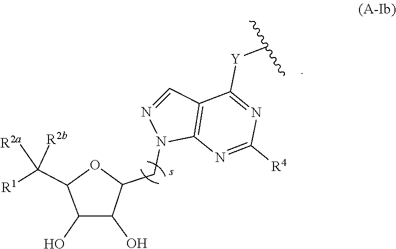

7. The conjugate of any one of claims 1-3, or a pharmaceutically acceptable salt thereof, wherein A¹ and A² each have the structure of formula (A-II):

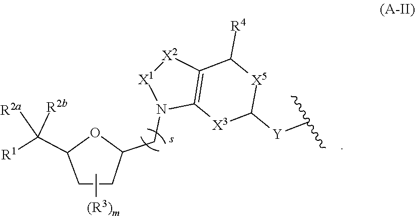

8. The conjugate of claim 7, or a pharmaceutically acceptable salt thereof, wherein A¹ and A² each have the structure of formula (A-IIa):

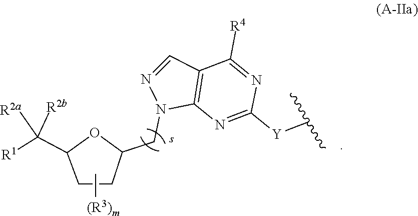

9. The conjugate of claim 8, or a pharmaceutically acceptable salt thereof, wherein A¹ and A² each have the structure of formula (A-IIb):

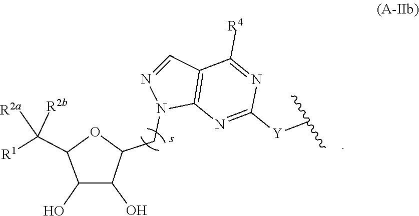

10. The conjugate of any one of claims 1-9, or a pharmaceutically acceptable salt thereof, wherein s is 0.

11. The conjugate of any one of claims 1-9, or a pharmaceutically acceptable salt thereof, wherein s is 1.

12. The conjugate of any one of claims 1-11, or a pharmaceutically acceptable salt thereof, wherein each of R^{2a} and R^{2b} is, independently, H, optionally substituted C₁-C₂₀ alkyl, or optionally substituted C₁-C₂₀ heteroalkyl.

13. The conjugate of claim 12, or a pharmaceutically acceptable salt thereof, wherein each of R^{2a} and R^{2b} is H.

14. The conjugate of any one of claims 1-13, or a pharmaceutically acceptable salt thereof, wherein R⁴ is H, halogen, OH, SH, optionally substituted amino, optionally substituted C₁-C₂₀ alkyl, or optionally substituted C₁-C₂₀ heteroalkyl.

15. The conjugate of claim 14, or a pharmaceutically acceptable salt thereof, wherein R⁴ is halogen.

16. The conjugate of claim 15, or a pharmaceutically acceptable salt thereof, wherein R⁴ is Cl.

17. The conjugate of any one of claims 1-16, or a pharmaceutically acceptable salt thereof, wherein R¹ is

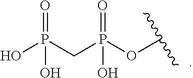 [SMILES: *OP(=O)(O)CP(=O)(O)O]

18. The conjugate of any one of claims 1-17, or a pharmaceutically acceptable salt thereof, wherein R¹ is

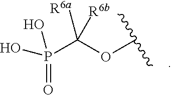

19. The conjugate of claim 18, or a pharmaceutically acceptable salt thereof, wherein each of R^{6a} and R^{6b} is, independently, H, optionally substituted C₁-C₂₀ alkyl, or optionally substituted C₁-C₂₀ heteroalkyl.

20. The conjugate of claim 19, or a pharmaceutically acceptable salt thereof, wherein each of R^{6a} and R^{6b} is, independently, H, —CH₃, —CH₂CH₃, —CH₂OH, —CH₂OCH₃—CH₂CH₂OH, or —CH₂CH₂OCH₃.

21. The conjugate of claim 20, or a pharmaceutically acceptable salt thereof, wherein each of R^{6a} and R^{6b} is H.

22. The conjugate of any one of claims 1-21, wherein Y is: 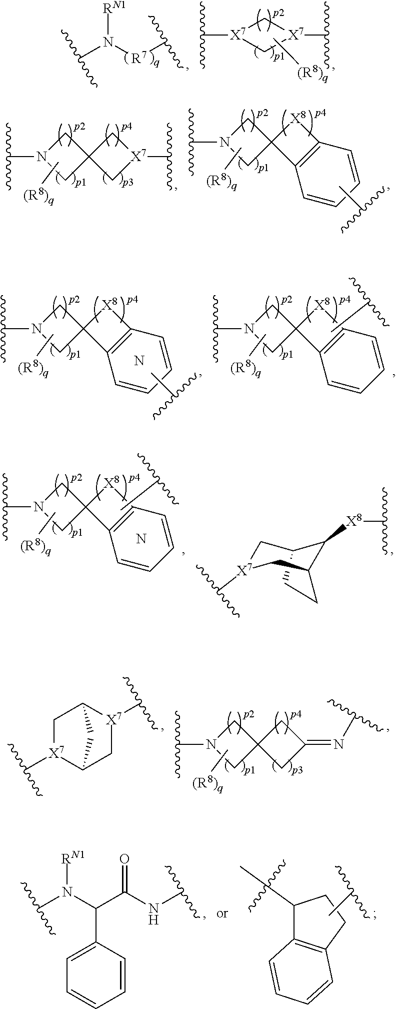
each of p1, p2, p3, and p4 is, independently, 0, 1, 2, 3, or 4;
q is 0, 1, 2, 3, or 4;
each X⁷ is, independently, N or CH;
each X⁸ is, independently, O, NH, CH₂, or C(═O);
R^{N1} is H, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, optionally substituted C₂-C₁₉ heteroaryl, optionally substituted C₁-C₂₀ alkaryl, or optionally substituted C₁-C₂₀ alkylcycloalkyl;
each R⁷ is, independently, 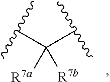
optionally substituted C₃-C₂₀ cycloalkyl, optionally substituted C₃-C₂₀ cycloalkenyl, optionally substituted C₂-C₂₀ heterocycloalkyl, or optionally substituted C₂-C₂₀ heterocycloalkenyl;
each of R^{7a} and R^{7b} is, independently, H, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl; and
each R⁸ is, independently, halogen, OH, SH, optionally substituted amino, optionally substituted C₁-C₂₀ alkyl, optionally substituted C₂-C₂₀ alkenyl, optionally substituted C₂-C₂₀ alkynyl, optionally substituted C₁-C₂₀ heteroalkyl, optionally substituted C₂-C₂₀ heteroalkenyl, optionally substituted C₂-C₂₀ heteroalkynyl, optionally substituted C₃-C₂₀ cycloalkyl, optionally substituted C₃-C₂₀ cycloalkenyl, optionally substituted C₂-C₂₀ heterocycloalkyl, optionally substituted C₂-C₂₀ heterocycloalkenyl, optionally substituted C₆-C₁₈ aryl, or optionally substituted C₂-C₁₉ heteroaryl.

23. The conjugate of claim 22, or a pharmaceutically acceptable salt thereof, wherein Y is

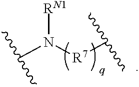

24. The conjugate of claim 23, or a pharmaceutically acceptable salt thereof, wherein q is 1 and Y is

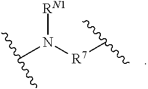

25. The conjugate of claim 24, or a pharmaceutically acceptable salt thereof, wherein R⁷ is 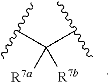
and Y is

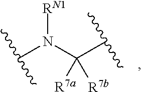

26. The conjugate of claim 24, or a pharmaceutically acceptable salt thereof, wherein Y is

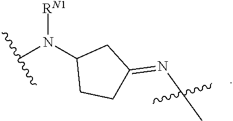

27. The conjugate of claim 23, or a pharmaceutically acceptable salt thereof, wherein q is 0 and Y is

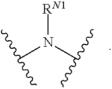

28. The conjugate of any one of claims 23-27, or a pharmaceutically acceptable salt thereof, wherein R^{N1} is H,

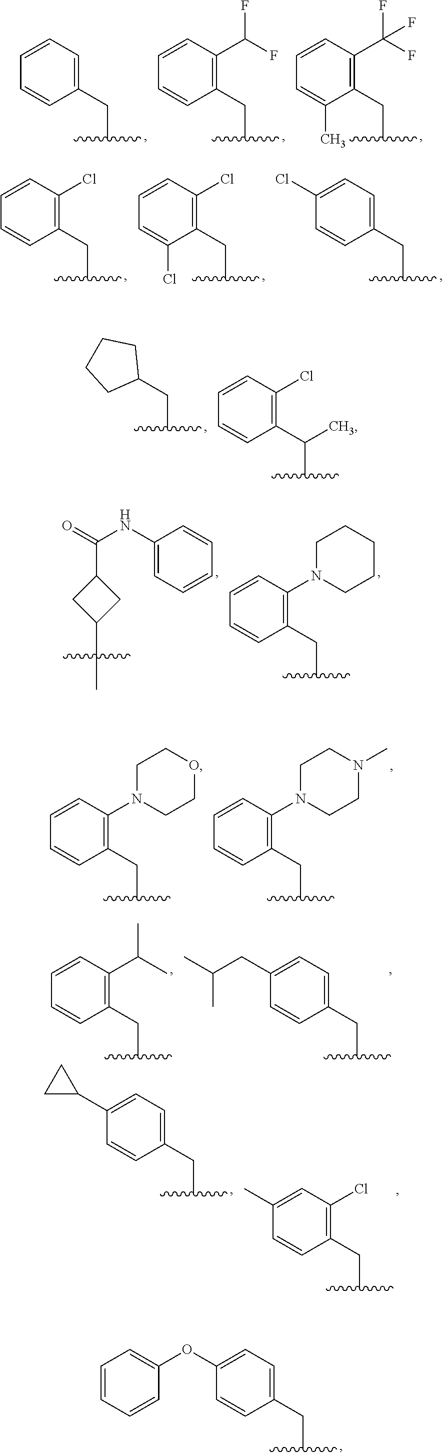

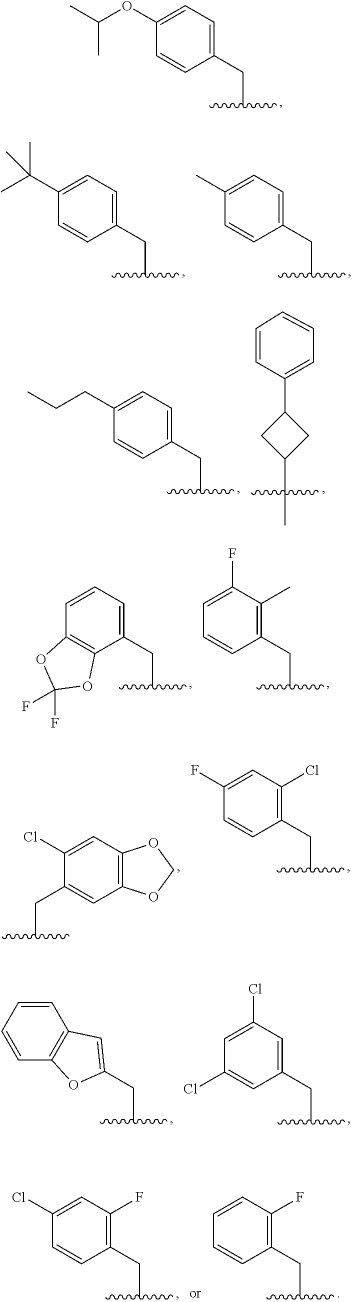

29. The conjugate of any one of claims 1-22, or a pharmaceutically acceptable salt thereof, wherein Y is

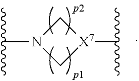

30. The conjugate of any one of claims 1-22, or a pharmaceutically acceptable salt thereof, wherein Y is

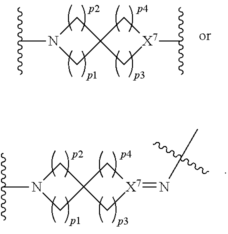

31. The conjugate of claim 30, or a pharmaceutically acceptable salt thereof, wherein Y is

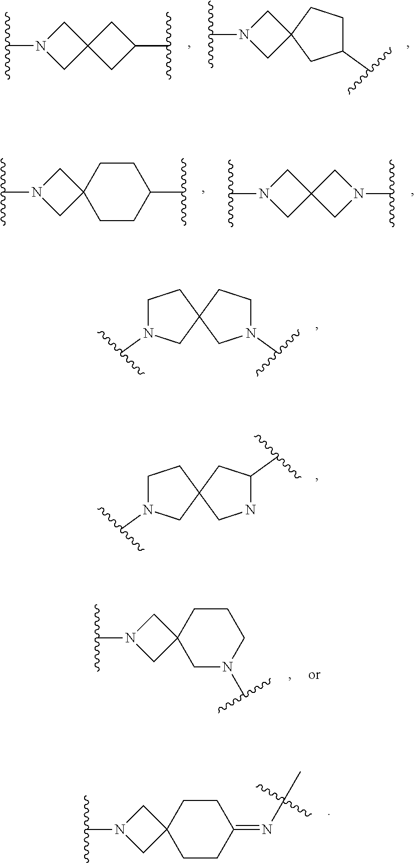

32. The conjugate of any one of claims 1-22, or a pharmaceutically acceptable salt thereof, wherein Y is

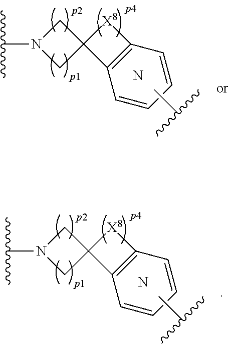

33. The conjugate of claim 32, or a pharmaceutically acceptable salt thereof, wherein Y is

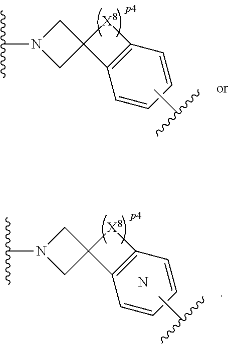

34. The conjugate of claim 33, or a pharmaceutically acceptable salt thereof, wherein Y is

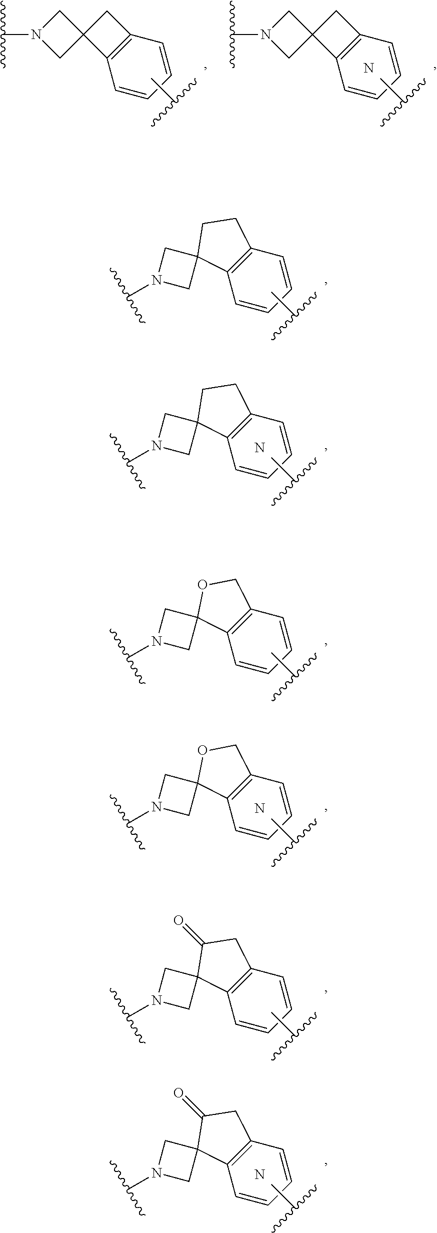

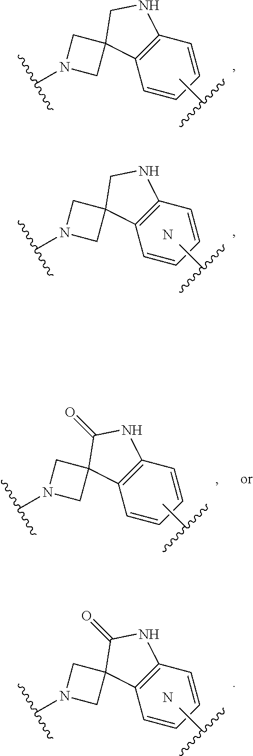

35. The conjugate of any one of claims 1-22, or a pharmaceutically acceptable salt thereof, wherein Y is

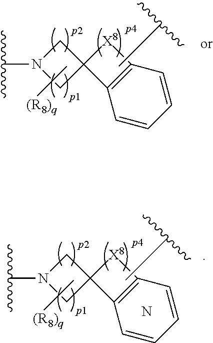

36. The conjugate of claim 35, or a pharmaceutically acceptable salt thereof, wherein Y is

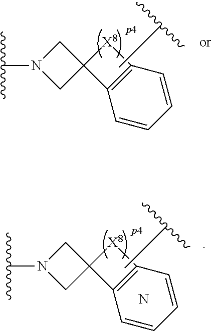

37. The conjugate of claim 36, or a pharmaceutically acceptable salt thereof, wherein Y is

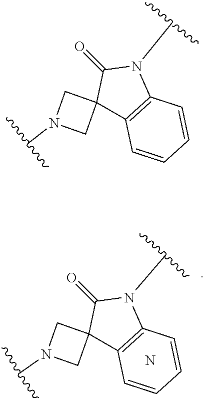

38. The conjugate of any one of claims 1-37, or a pharmaceutically acceptable salt thereof, wherein L comprises one or more optionally substituted C₁-C₄₀ alkylene, optionally substituted C₁-C₄₀ heteroalkylene, optionally substituted C₁-C₄₀ alkoxylene, optionally substituted C₂-C₂₀ alkenylene, optionally substituted C₂-C₂₀ heteroalkenylene, optionally substituted C₂-C₂₀ alkynylene, optionally substituted C₂-C₂₀ heteroalkynylene, optionally substituted C₃-C₂₀ cycloalkylene, optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₈-C₂₀ heterocycloalkynylene, optionally substituted C₅-C₁₅ arylene, optionally substituted C₂-C₁₅ heteroarylene, O, S, NRⁱ, P, carbonyl, thiocarbonyl, sulfonyl, phosphate, phosphoryl, or imino,
wherein R¹ is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁s aryl, or optionally substituted C₂-C₁₅ heteroaryl.

39. The conjugate of any one of claims 1-38, or a pharmaceutically acceptable salt thereof, wherein L is oxo substituted.

40. The conjugate of any one of claims 1-39, or a pharmaceutically acceptable salt thereof, wherein the backbone of L comprises between 1 and 250 atoms.

41. The conjugate of any one of claims 1-40, or a pharmaceutically acceptable salt thereof, wherein L is capable of forming an amide, a carbamate, a sulfonyl, or a urea linkage.

42. The conjugate of any one of claims 3-41, or a pharmaceutically acceptable salt thereof, wherein L is described by the formula: 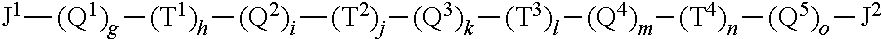
wherein J¹ is a bond attached to A₁;
J² is a bond attached to E or is a functional group capable of reacting with a functional group conjugated to E;
each of Q¹, Q², Q³, Q⁴ and Q⁵ is, independently, optionally substituted C1-C40 alkylene, optionally substituted C1-C40 heteroalkylene, optionally substituted C1-C40 alkoxylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, or optionally substituted C2-C15 heteroarylene;
each of T¹, T², T³, T⁴ is, independently, O, S, NRⁱ, P, carbonyl, thiocarbonyl, sulfonyl, phosphate, phosphoryl, imino, or oximo;
Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl; and
each of g, h, i, j, k, l, m, n, and o is, independently, 0, 1, or 2.

43. The conjugate of claim 42, or a pharmaceutically acceptable salt thereof, wherein Q¹ is

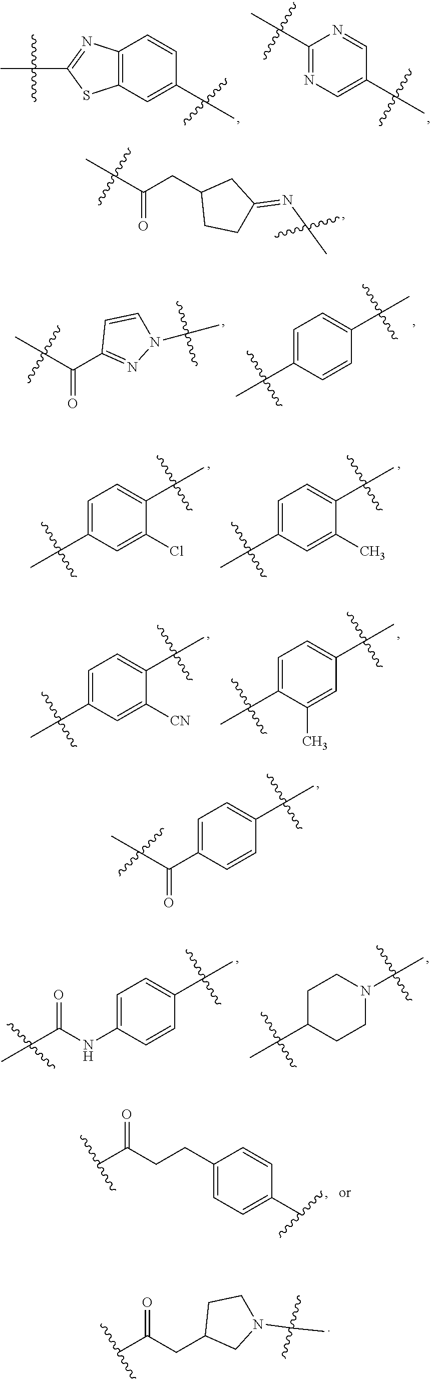

44. The conjugate of claim 42 or 43, or a pharmaceutically acceptable salt thereof, wherein Q² is optionally substituted C₁-C₄₀ alkylene, optionally substituted C₁-C₄₀ heteroalkylene, optionally substituted C₁-C₄₀ alkoxylene, or optionally substituted C₂-C₁₅ heteroarylene.

45. The conjugate of any one of claims 42-44, or a pharmaceutically acceptable salt thereof, wherein Q³ is optionally substituted C₂-C₁₅ heteroarylene.

46. The conjugate of any one of claims 42-45, or a pharmaceutically acceptable salt thereof, wherein Q⁴ is optionally substituted C₁-C₄₀ alkylene, optionally substituted C₁-C₄₀ heteroalkylene, or optionally substituted C₁-C₄₀ alkoxylene.

47. The conjugate of any one of claims 42-46, or a pharmaceutically acceptable salt thereof, wherein J² is

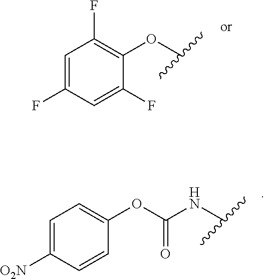 [SMILES: *Oc1c(F)cc(F)cc1F ; *NC(=O)Oc1ccc([N+](=O)[O-])cc1]

48. A conjugate, or a pharmaceutically acceptable salt thereof, described by a formula of Table 2.

49. The conjugate of any one of claims 1-48, or a pharmaceutically acceptable salt thereof, wherein the squiggly line connected to E indicates that the L of each A₁-L or each A1-L-A₂ is covalently attached to a nitrogen atom of a solvent-exposed lysine of E.

50. The conjugate of any one of claim 1-48, or a pharmaceutically acceptable salt thereof, wherein the squiggly line connected to E indicates that the L of each A₁-L or each A1-L-A₂ is covalently attached to the sulfur atom of a solvent-exposed cysteine of E.

51. The conjugate of any one of claims 1-48, or a pharmaceutically acceptable salt thereof, wherein n is 2, and each E dimerizes to form an Fc domain.

52. The conjugate of any one of claims 1-51, or a pharmaceutically acceptable salt thereof, wherein each E is a human IgG1 Fc domain monomer.

53. The conjugate of any one of claims 1-52, or a pharmaceutically acceptable salt thereof, wherein each E comprises a substitution mutation at N297 selected from N297A, N297G, or N297Q, wherein the amino acid numbering of each Fc domain monomer is according to the Kabat EU index.

54. The conjugate of any one of claims 1-53, or a pharmaceutically acceptable salt thereof, wherein each E comprises a C220S substitution mutation, wherein the amino acid numbering of each Fc domain monomer is according to the Kabat EU index.

55. The conjugate of any one of claims 1-54, or a pharmaceutically acceptable salt thereof, wherein each E comprises a M252Y, a S254T, and a T256E substitution mutation, wherein the amino acid numbering of each Fc domain monomer is according to the Kabat EU index.

56. The conjugate of any one of claims 1-52, or a pharmaceutically acceptable salt thereof, wherein each E comprises the amino acid sequence of any one of SEQ ID NOs: 1-112 or 115-120.

57. The conjugate of claim 56, or a pharmaceutically acceptable salt thereof, wherein each E comprises the sequence of SEQ ID NO: 13, SEQ ID NO: 14, SEQ ID NO: 17, or SEQ ID NO: 18.

58. The conjugate of claim 56, or a pharmaceutically acceptable salt thereof, wherein each E comprises the sequence of SEQ ID NO: 80, SEQ ID NO: 81, SEQ ID NO: 82, SEQ ID NO: 83, SEQ ID NO: 116, or SEQ ID NO: 120.

59. The conjugate of any one of claims 1-58, or a pharmaceutically acceptable salt thereof, wherein T is 1, 2, 3, 4, 5, 6, 7, 8, 9, or 10.

60. A population of conjugates of any one of claims 1-59, or a pharmaceutically acceptable salt thereof, wherein the average value of T is 1 to 10.

61. A population of conjugates of claim 60, or a pharmaceutically acceptable salt thereof, wherein the average value of T is 1 to 5.

62. A pharmaceutical composition comprising a conjugate or a population of conjugates of any one of claims 1-61, or a pharmaceutically acceptable salt thereof, and a pharmaceutically acceptable excipient.

63. A method of treating a cancer in a subject, the method comprising administering to the subject a conjugate, population of conjugates, or pharmaceutical composition of any one of claims 1-62.

64. The method of claim 63, wherein the cancer is selected from lung cancer, optionally non-small cell lung cancer or small-cell lung cancer; head and neck cancer, optionally squamous cell carcinoma; renal cell carcinoma; breast cancer; ovarian cancer; pancreatic cancer; colorectal cancer; urothelial cancer; bile duct cancer; endometrial cancer; melanoma; or esophageal cancer.

65. The method of claim 63 or 64, wherein the cancer is a solid tumor.

66. The method of any one of claims 63-65, wherein the cancer overexpresses or is known to overexpress CD73 relative to a non-cancerous cell of the same tissue type.

67. The method of any one of claims 63-66, further comprising administering to the subject an immune checkpoint inhibitor.

68. The method of claim 67, wherein the immune checkpoint inhibitor is a PD-1 inhibitor.

69. A method of treating or preventing a viral infection in a subject, the method comprising administering to the subject a conjugate, population of conjugates, or pharmaceutical composition of any one of claims 1-62.

70. The method of claim 69, wherein the viral infection is a betacoronavirus infection.

71. The method of claim 70, wherein the betacoronavirus is SARS-CoV-2.

72. The method of claim 71, wherein the SARS-CoV-2 is an Alpha, Delta, or Omicron variant.

73. The method of claim 72, wherein the SARS-CoV-2 is an Omicron variant.

74. The method of claim 73, wherein the Omicron variant is a BA.1, BA.2, BA.3, BA.4, or BA.5 lineage.

75. The method of any one of claims 69-74, wherein the method further comprises administering to the subject an antiviral agent or an antiviral vaccine.

76. A method of treating or preventing fibrosis in a subject, the method comprising administering to the subject a conjugate, population of conjugates, or pharmaceutical composition of any one of claims 1-62.

77. The method of claim 76, wherein the fibrosis is pulmonary fibrosis, dermal fibrosis, renal fibrosis, hepatic fibrosis, cardiac fibrosis, or systemic sclerosis.

78. The method of claim 77, wherein the fibrosis is pulmonary fibrosis.

79. The method of claim 78, wherein the pulmonary fibrosis is associated with a viral infection, drug-induced pulmonary fibrosis, radiation-induced pulmonary fibrosis, hypersensitivity pneumonitis, idiopathic pulmonary fibrosis, non-specific interstitial pneumonia, pneumoconiosis, interstitial lung disease, sarcoidosis, silicosis, or systemic sclerosis.

80. The method of any one of claims 63-79, wherein the conjugate, population of conjugates, or pharmaceutical composition is administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally, locally, by inhalation, by injection, or by infusion.
